(19) United States
(12) Patent Application Publication
(10) Pub. No.: US 2021/0371932 A1
(43) Pub. Date: Dec. 2, 2021

(54) METHODS AND COMPOSITIONS FOR DETECTING AND MODULATING MICROENVIRONMENT GENE SIGNATURES FROM THE CSF OF METASTASIS PATIENTS

(71) Applicants: Massachusetts Institute of Technology, Cambridge, MA (US); The General Hospital Corporation, Boston, MA (US); Dana-Farber Cancer Institute, Inc., Boston, MA (US)

(72) Inventors: Alexander K. Shalek, Cambridge, MA (US); Sanjay Prakadan, Cambridge, MA (US); Priscilla K. Brastianos, Boston, MA (US); Christopher Allen Alvarez-Breckenridge, Boston, MA (US); Scott Carter, Boston, MA (US)

(21) Appl. No.: 15/733,925

(22) PCT Filed: Jun. 3, 2019
(86) PCT No.: PCT/US2019/035252

§ 371 Date: Dec. 1, 2020

Related U.S. Application Data
(60) Provisional application No. 62/679,728, filed on Jun. 1, 2018.

(51) Int. Cl. C12Q 1/6886

(52) U.S. Cl. CPC C12Q 1/6886; C12Q 2600/106; C12Q 2600/158

(57) ABSTRACT

The subject matter disclosed herein is generally directed to detecting and modulating novel gene signatures for the treatment and prognosis of cancer. The gene signatures can be detected in samples obtained from the extracellular fluid of a subject suffering from cancer. The novel gene signatures can be used to predict and monitor responses to immunotherapy in cancer and can be targeted therapeutically. The immunotherapy can be immune checkpoint inhibition therapy.

### CROSS-REFERENCE TO RELATED APPLICATIONS

This application claims the benefit of U.S. Provisional Application No. 62/679,728, filed Jun. 1, 2018. The entire contents of the above-identified application are hereby fully incorporated herein by reference.

### REFERENCE TO AN ELECTRONIC SEQUENCE LISTING

The contents of the electronic sequence listing (BROD_2620WP.ST25.txt”; Size is 6,993 bytes and it was created on Jun. 3, 2019) is herein incorporated by reference in its entirety.

### TECHNICAL FIELD

The subject matter disclosed herein is generally directed to detecting and modulating novel gene signatures for the treatment and prognosis of cancer.

### BACKGROUND

Brain metastasis presents an important obstacle to cancer survival. With 100,000 new cases annually and a median survival of 4-6 months, brain metastasis research is of paramount importance (Eichler, A. F. et al. The biology of brain metastases—translation to new therapies. Nat Rev Clin Oncol 8, 344-356 (2011); and Owonikoko, T. K. et al. Current approaches to the treatment of metastatic brain tumours. Nat Rev Clin Oncol 11, 203-222, doi:10.1038/nrclinonc.2014.25 (2014)).

Currently, major recourses for patients with brain metastases—whole brain radiation therapy and surgery—are invasive and dangerous. Hence, efforts are needed to realize safer interventions, and to define biomarkers for diagnosis and monitoring.

Two major hurdles exist in characterizing brain metastases. First, tumors are complex cellular mixtures (Sharma, S. V. et al. A chromatin-mediated reversible drug-tolerant state in cancer cell subpopulations. Cell 141, 69-80, doi:10.1016/j.cell.2010.02.027 (2010); Spencer, S. L., Gaudet, S., Albeck, J. G., Burke, J. M. & Sorger, P. K. Non-genetic origins of cell-to-cell variability in TRAIL-induced apoptosis. Nature 459, 428-432, doi:10.1038/nature08012 (2009); and Patel, A. P. et al. Single-cell RNA-seq highlights intratumoral heterogeneity in primary glioblastoma. Science 344, 1396-1401, doi:10.1126/science.1254257 (2014)), limiting the utility of bulk profiling and impacting therapeutic efficacy (Tirosh, I. et al. Dissecting the multicellular ecosystem of metastatic melanoma by single-cell RNA-seq. Science 352, 189-196, doi:10.1126/science.aad0501 (2016)). The emergence of scRNA-Seq now enables unprecedented deconvolution of this heterogeneity through direct genome-wide examination of individual cells, yielding clinically-relevant, actionable results (Shalek, A. K. et al. Single-cell transcriptomics reveals bimodality in expression and splicing in immune cells. Nature 498, 236-240, doi:10.1038/nature12172 (2013); and Shalek, A. K. et al. Single-cell RNA-seq reveals dynamic paracrine control of cellular variation. Nature 510, 363-369, doi:10.1038/nature13437 (2014)). Second, acquiring metastatic tissue from the brain is difficult. Surgical resections are often “one-time” events from late-stage patients, prohibiting longitudinal studies of treatment response. In recent years, liquid biopsies have emerged as a powerful diagnostic; for brain metastases, CSF provides a unique, relevant source of cells (Sevenich, L. et al. Analysis of tumour- and stroma-supplied proteolytic networks reveals a brain-metastasis-promoting role for cathepsin S. Nat Cell Biol 16, 876-888, doi:10.1038/ncb3011 (2014)).

Over the past decade, immunotherapy has emerged as a promising strategy for the treatment of cancer. Unlike conventional tactics which directly target tumor cell viability, this class of approaches instead reinvigorates the immune system to facilitate eradication. As such, it has the potential to be broadly applicable, given the existence of conserved mechanisms for downregulating immune activity against “self” that are hijacked by many tumors to evade immunity. Among the immunotherapies, one set that has shown particularly striking clinical activity against a range of tumors is checkpoint blockade inhibitors (CBIs). These molecules—typically antibodies—act by binding inhibitory co-receptors on T cells, such as PD-1 and CTLA-4, that are often engaged by tumor cells (or their tumor cell cognates), to restore immune function. In particular, checkpoint blockade inhibitors (CBIs) lead to durable responses in ˜35% of patients with metastatic melanoma by unleashing T cells from oncogenic suppression (P. Sharma, J. P. Allison, The future of immune checkpoint therapy. Science. 348, 56-61 (2015); and F. S. Hodi, Kluger H M, Sznol M, Durable, long-term survival in previously treated patients with advanced melanoma who received nivolumab monotherapy in a phase I trial. 2016 AACR Annu. Meet. Abstr. CT001 Present. Apr. 17, 2016). Yet, despite early successes with CBIs, their broad applicability has been challenged by mounting observations of partial response, acquired resistance, and inconsistent benefit across patients with the same tumor type. Understanding the cellular and molecular mechanisms that result in response or resistance to immunotherapy is critical for realizing its full therapeutic potential. This, in turn, requires a detailed understanding of the changes induced throughout the tumor microenviroment (TME) upon CBI introduction, as well as their potential relationship to clinical response.

One of the most direct means by which to achieve this knowledge is to extensively profile a tumor before and after introduction of CBIs. Given potentially confounding differences between patients in disease etiology and response dynamics, this would ideally be done within an individual to decouple the effects of CBI introduction from other axes of uncontrolled variation—both known and unknown—and guided by clinical observations of contemporaneous patient state. Similarly, to avoid sampling artifacts, this profiling would be done within the same TME in a manner that could resolve the unique contributions of the myriad constituent cells to overall response. Unfortunately, given the invasiveness of conventional sampling methods (e.g., resections or core biopsies) or the paucity of material recovered via their alternatives (e.g., needle aspirates), it has been challenging to comprehensively characterize the TME before and after treatment.

Citation or identification of any document in this application is not an admission that such document is available as prior art to the present invention.

### SUMMARY

Immunotherapies, such as checkpoint blockade inhibitors (CBIs) produce durable responses in some cancer patients, yet most patients derive no clinical benefit. The molecular underpinnings of CBI resistance (ICR) are elusive. It is an objective of the present invention to identify molecular signatures for diagnosis, prognosis and treatment of subjects suffering from cancer. It is a further objective to understand tumor immunity and to leverage this knowledge for treating subjects suffering from cancer. It is another objective for identifying gene signatures for predicting response to checkpoint blockade therapy. It is another objective, for modulating the molecular signatures in order to increase efficacy of immunotherapy (e.g., checkpoint blockade therapy).

In one aspect, the present invention provides for a method for detecting, monitoring or prognosing a cancer in a subject in need thereof comprising: obtaining a biological sample comprising tumor cells from an extracellular fluid or compartment of the subject; detecting in the biological sample the expression or activity of a gene signature associated with sensitivity to a therapy or response to a therapy; and determining that the solid tumor present, is responding to a therapy or is capable of responding to a therapy. In certain embodiments, the gene signature is detected in single cells from the biological sample. In certain embodiments, the biological sample comprising tumor cells is obtained from cerebral spinal fluid (CSF). In certain embodiments, the biological sample comprising tumor cells is obtained from draining lymph nodes.

In certain embodiments, the gene signature is associated with a response to immunotherapy. The immunotherapy may comprise one or more check point inhibitors. The one or more check point inhibitors may comprise anti-CTLA4, anti-PD-L1 and/or anti-PD1 therapy. In certain embodiments, the signature exhibits upregulation of (i) genes involved in interferon regulation; (ii) genes involved in interferon response; (iii) genes involved in antigen presentation; and/or (iv) genes involved in cytotoxic T cell activation compared to the transcriptome profile of the reference sample. In certain embodiments, the gene signature comprises one or more genes or polypeptides selected from the group consisting of: AHNAK, ANKRD30B, BTG1, BZW1, C8orf4, COX6C, DSP, DUSP1, EEF1A1, EEF1D, EGR1, EIF2S2, FOS, FOSB, FTH1, GOLGB1, HES1, IRX2, JUN, JUNB, MALAT1, MGEA5, MLPH, MORF4L1, NDRG1, NEAT1, NR4A2, NUPR1, RB1CC1, RNA28S5, RPL10, RPL10A, RPL12, RPL13A, RPL18A, RPL22, RPL23A, RPL26, RPL3, RPL36, RPL37, RPL4, RPL41, RPL6, RPS15, RPS15A, RPS18, RPS2, RPS20, RPS25, RPS27, RPS27A, RPS28, RPS29, RPS7, RPSA, S100A8, S100A9, SCGB1D2, SLK, SNHG9, SRRM2, TMEM14C, TRIB1, TRPS1, UBC, UQCRB and ZFAS1; or AARD, ACP1, ACTR2, ACTR3, ACTR6, AIMP1, ANP32B, AP2M1, APIP, APOD, APOL6, ARF1, ATAD2, ATP5G3, B2M, C1orf43, CALM1, CCT3, CCT4, CCT5, CCT6A, CD44, CD46, CD74, CHCHD2, COA6, COPS2, COX7A2, COX7C, COX8A, CP, EBNA1BP2, EIF2AK2, EIF5, EPRS, EZH2, FKBP3, GBP1, GTF3A, H2AFZ, HDGF, HIST1H1D, HIST1H4C, HLA-B, HLA-C, HNRNPA2B1, HSBP1, HSP90B1, IFI16, IFI27, IFI44L, IFI6, IFIH1, IFIT3, IFNAR1, IL6ST, ISG15, LAMP2, LMAN1, MAPKAP1, MDH1, MED10, MGP, MRPL13, MX1, NDUFA1, NDUFA4, NDUFB2, NUCB2, OAZ1, PAICS, PALLD, PAPOLA, PARP1, PARP9, PDCD5, PDHX, PDIA6, PEG10, PGRMC1, PKIB, PPM1G, PPP1CB, PSMA3, PSMB8, PSME1, PSME2, PTGES3, RAN, RARRES1, RHOA, RSRC1, S100A6, SEC61B, SEC62, SIVA1, SLC38A1, SMARCA5, SNX6, SRP72, SSR3, SSR4, STMN1, TAP1, TCP1, TM4SF1, TMCO1, TMEM59, TMEM97, TMPO, TRAM1, TSPO, TXNL1, UBE2J1, UBL5, UCHL5, UQCRH, USP1, VBP1 and XAF1; or ACTB, AHNAK, BTG1, BZW1, C6orf62, C8orf59, COX6C, DANCR, DUSP1, EEF1B2, EEF1D, EGR1, EIF2S2, FOS, FTH1, FTL, HES1, HNRNPA1, JUNB, METTL12, MIF, MORF4L1, MT-ATP6, MT-ATP8, MT-CO1, MT-CO2, MT-CO3, MT-CYB, MT-ND1, MT-ND2, MT-ND4, MT-ND4L, MT-ND5, MT-ND6, MT-RNR1, MT-TL1, MT-TV, MTRNR2L1, MTRNR2L11, MTRNR2L12, MTRNR2L2, MTRNR2L3, MTRNR2L8, NDUFB1, NEAT1, NR4A2, PCBP2, PET100, PFDN5, PPDPF, PRDX2, RB1CC1, RNA18S5, RNA28S5, RNY1, ROMO1, RPL10, RPL10A, RPL12, RPL13, RPL13A, RPL15, RPL18, RPL18A, RPL22, RPL23A, RPL26, RPL27A, RPL28, RPL29, RPL31, RPL32, RPL34, RPL35, RPL36, RPL37, RPL39, RPL4, RPL41, RPL6, RPL7A, RPL8, RPL9, RPN2, RPS14, RPS15, RPS15A, RPS18, RPS19, RPS2, RPS20, RPS25, RPS27, RPS27A, RPS28, RPS29, RPS3, RPS3A, RPS4X, RPS7, RPS9, RPSA, S100A8, SLK, SNHG9, SYNGR2, TMA7, TMSB10, TMSB4X, UBC and UQCRB; or ACBD3, ACP1, ACTR2, ACTR3, ACTR6, ADSS, AMD1, AP2M1, APEX1, ARF1, ARPC5, ASH1L, ATAD2, ATP5G3, B2M, BROX, C1orf21, C1orf43, CALM1, CANX, CAPN2, CAPRIN1, CCDC59, CCNC, CCT2, CCT3, CCT4, CCT6A, CD109, CD46, CDC16, CDCA7L, CLTC, CMPK1, CNBP, COA6, COPS2, CP, DENND1B, DHX9, DNAJC3, DST, DSTN, ECT2, EID1, EIF2AK2, EIF2S1, EIF3D, EIF4A2, EIF5, EMC4, ENO1, EPRS, EZH2, FKBP3, GADD45GIP1, GMPS, GTF3A, GTPBP4, HADHA, HDGF, HDLBP, HIST1H1D, HIST1H4C, HNRNPA2B1, HSP90B1, HSPA5, HSPH1, IFNAR1, IQGAP1, LAMP2, LMAN1, LPP, LRPPRC, MAN1A2, MAPKAP1, MBNL1, MBNL2, MED10, MORF4L2, MRPL13, MRPL42, MRPS35, MX1, NASP, NOL11, NSMCE2, NUCB2, NUCKS1, NUDCD1, PAICS, PAPOLA, PARP1, PDCD10, PDHX, PDIA6, PGRMC1, PIK3R1, PLRG1, PMAIP1, PMP22, POLR2B, PPM1G, PRPF40A, PSMA3, PSME4, PTBP3, PTTG1IP, RAB10, RAD21, RAN, RARRES1, RHOA, RNF168, RSRC1, S100A6, SAP30BP, SCRN1, SEC63, SENP6, SF3B1, SLC38A1, SMARCA5, SMC3, SMEK2, SNX6, SPEN, SRP72, SRSF3, SSR3, SSRP1, ST3GAL1, SUCO, TCP1, TKT, TM4SF1, TMPO, TOMM20, TOMM70A, TP53BP2, TPM1, TRAM1, TXNL1, UBE2J1, UBE2K, UCHL5, UGP2, USP1, USP16, VBP1, VIM, XIST, ZC3H14 and ZDHHC20; or COX6C, EEF1D, RNA28S5, RPL10, RPL12, RPL13A, RPL18A, RPL23A, RPL26, RPL36, RPL41, RPL6, RPS15, RPS15A, RPS18, RPS27, RPS27A, RPS28, RPS29 and RPS7; or ACTR3, ARF1, CCT4, CCT6A, COPS2, EIF2AK2, EPRS, HDGF, HIST1H1D, HIST1H4C, HNRNPA2B1, HSP90B1, LAMP2, MRPL13, PAPOLA, PPM1G, RHOA, SLC38A1, SMARCA5, SRP72, SSR3, TCP1, TRAM1 and TXNL1; or ACTB, ACTN4, AP2S1, APH1A, ATP5I, ATP5J2, ATP5L, C19orf48, C4orf48, C6orf62, C8orf59, CDC37, CDKN2A, CEBPB, CFL1, CHCHD2, CHCHD3, CKB, COX5B, COX6A1, COX6C, COX7A2, COX7B, DANCR, DUSP18, EEF1B2, EEF1D, FAU, FTL, FXYD5, GPX1, GSTP1, HAP1, HNRNPA1, JUP, LGALS1, LHCGR, LSM4, LSM7, METTL12, MIF, MLF2, MRPS12, MRPS26, MT-ATP6, MT-ATP8, MT-CO1, MT-CO2, MT-CO3, MT-CYB, MT-ND1, MT-ND2, MT-ND4, MT-ND4L, MT-ND5, MT-ND6, MT-RNR1, MT-TL1, MT-TV, MTRNR2L1, MTRNR2L11, MTRNR2L12, MTRNR2L13, MTRNR2L2, MTRNR2L3, MTRNR2L5, MTRNR2L6, MTRNR2L7, MTRNR2L8, MZT2B, NDUFA11, NDUFA13, NDUFA2, NDUFB1, NDUFB4, OAZ1, PCBP1, PCBP2, PET100, PFDN5, PFN1, POLR2J, PPDPF, PRDX2, PSMB3, RAB32, RHOC, RMRP, RNA18S5, RNA28S5, RNY1, ROMO1, RPL10, RPL12, RPL13, RPL13A, RPL15, RPL18, RPL18A, RPL23A, RPL24, RPL26, RPL27A, RPL28, RPL29, RPL31, RPL32, RPL34, RPL35, RPL36, RPL36AL, RPL39, RPL41, RPL6, RPL7A, RPL8, RPL9, RPN2, RPS14, RPS15, RPS15A, RPS18, RPS19, RPS27, RPS27A, RPS28, RPS29, RPS3, RPS3A, RPS4X, RPS7, RPS9, S100A11, S100A13, SERF2, SNHG5, SSR4, SYNGR2, TAGLN2, TIMM8B, TMA7, TMSB10, TMSB4X, TRIM28, TTC19, TUBA1A, TUBA1B, UBA52 and UBL5; or ABI2, ACBD3, ACIN1, ACSL4, ACTR3, ADK, ADSS, AMD1, ANKRD17, APEX1, AQR, ARF1, ARFGEF2, ARHGEF12, ARPC5, ASH1L, ASPH, ATL3, ATP1A1, ATP2A2, BNIP3, BROX, BRWD1, C1orf21, CAAP1, CANX, CAPN2, CAPRIN1, CBX5, CCDC59, CCDC90B, CCNC, CCT2, CCT4, CCT6A, CD109, CDC16, CDC5L, CDCA7L, CEBPG, CHEK1, CHML, CLIC4, CLTC, CMPK1, CNBP, COPB1, COPS2, CTTNBP2NL, CUL5, DARS, DCBLD1, DENND1B, DHRS7, DHX36, DHX40, DHX9, DICERI, DNAJC10, DNAJC3, DPH3, DST, DSTN, ECT2, EID1, EIF2AK1, EIF2AK2, EIF2S1, EIF3D, EIF4A2, EMC4, ENDOD1, ENO1, EPRS, EPS8, ERC1, ERRFI1, ETF1, EXOC3, FAM208B, FAR1, FNDC3A, FUBP1, G3BP2, GADD45GIP1, GDI2, GLTSCR2, GMPS, GOLGB1, GTPBP4, H2AFY, H3F3B, HADHA, HDGF, HDLBP, HIST1H1B, HIST1H1C, HIST1H1D, HIST1H1E, HIST1H4C, HIST2H2AC, HNRNPA2B1, HNRNPUL1, HSP90B1, HSPA5, HSPH1, IARS, ILF3, INO80D, IQGAP1, ITGA2, KIAA0368, KIAA0947, KIAA1429, KIF1B, KIF2A, LAMP2, LARP4, LARS, LCOR, LEO1, LPP, LRPPRC, LTN1, MAN1A2, MAP1LC3B, March7, MARK3, MBD4, MBNL1, MBNL2, MCUR1, MDN1, MGA, MGEA5, MIB1, MOB1B, MORF4L2, MRPL13, MRPL42, MRPS35, MSANTD3, MYCBP2, MYSM1, NASP, NCL, NEDD4L, NET1, NFX1, NOL11, NSMCE2, NUCKS1, NUDCD1, NUDT21, PAIP2, PAPOLA, PBX1, PCYT1A, PDCD10, PDE4DIP, PIK3R1, PIK3R3, PLRG1, PMAIP1, PMP22, PNPLA8, POLR2B, PPFIA1, PPM1G, PPP2R2D, PPP4R1, PRPF40A, PSMD2, PSME4, PTBP3, PTPLB, PTPN1, PTTG1IP, RAB10, RAB2A, RABEP1, RAD21, RBM28, REEP3, RHOA, RNF115, RNF168, SAP30BP, SCRN1, SEC63, SENP2, SENP6, SETD3, SETX, SF3B1, SFSWAP, SH3GLB1, SLC12A2, SLC38A1, SLC4A7, SMARCA5, SMARCC1, SMC3, SMEK1, SMEK2, SMNDC1, SNRNP200, SNX4, SOCS4, SPEN, SRP72, SRRM2, SRSF3, SSR3, SSRP1, ST3GAL1, STK3, SUCO, TAF1D, TAF2, TCP1, TIMMDC1, TIPARP, TKT, TLK1, TM9SF3, TOMM20, TOMM70A, TP53BP2, TPM1, TRA2A, TRAM1, TRIP12, TUG1, TXNL1, UAP1, UBA6, UBAP1, UBAP2, UBE2H, UBE2K, UBTF, UBXN4, UGP2, USP14, USP16, USP34, USP9X, UTP20, VIM, WDR3, XIST, XRN2, YES1, YWHAH, ZC3H14, ZDHHC20, ZFAS1, ZFYVE16 and ZNF638; or any combination of up and down regulated genes in any of the Tables herein.

In another aspect, the present invention provides for a method of detecting an immunotherapy response gene signature in a tumor comprising, detecting in tumor cells obtained from a subject in need thereof the expression or activity of a gene signature according to claim 9.

In another aspect, the present invention provides for a method of treating a cancer in a subject in need thereof comprising detecting the expression or activity of a gene signature according to claim 9 in a biological sample comprising tumor cells obtained from the subject and administering a treatment, wherein if a gene signature associated with non-response to an immunotherapy is detected the treatment comprises administering an agent capable of reducing expression or activity of said signature or increasing expression or activity of a gene signature associated with response to an immunotherapy, and wherein if a gene signature associated with response to an immunotherapy is detected the treatment comprises administering an immunotherapy. In certain embodiments, the agent modulates expression or activity of one or more genes according to claim 9. In certain embodiments, the agent is capable of targeting or binding to one or more up-regulated secreted or cell surface exposed signature genes or polypeptides. The agent may comprise a therapeutic antibody, antibody fragment, antibody-like protein scaffold, aptamer, protein, genetic modifying agent or small molecule. The method may further comprise administering an immunotherapy to the subject administered an agent capable of reducing the expression or activity of said signature. The immunotherapy may comprise one or more check point inhibitors. The one or more check point inhibitors may comprise anti-CTLA4, anti-PD-L1 and/or anti-PD1 therapy.

In another aspect, the present invention provides for a method of treating a cancer in a subject in need thereof comprising administering to the subject a therapeutically effective amount of an agent: capable of modulating the expression or activity of one or more signature genes or polypeptides according to claim 9; or capable of targeting or binding to one or more cell surface exposed one or more signature genes or polypeptides according to claim 9; or capable of targeting or binding to one or more receptors or ligands specific for a cell surface exposed one or more signature genes or polypeptides according to claim 9; or comprising one or more secreted signature genes or polypeptides according to claim 9; or capable of targeting or binding to one or more secreted one or more signature genes or polypeptides according to claim 9; or capable of targeting or binding to one or more receptors specific for one or more secreted signature genes or polypeptides according to claim 9. The agent may comprise a therapeutic antibody, antibody fragment, antibody-like protein scaffold, aptamer, protein, genetic modifying agent or small molecule. The method may further comprise administering an immunotherapy to the subject. The immunotherapy may comprise a check point inhibitor. The checkpoint inhibitor may comprise anti-CTLA4, anti-PD-L1 and/or anti-PD1 therapy.

In certain embodiments, the cancer is a metastatic solid cancer that has metastasized to the brain. The metastatic solid cancer may have metastasized from a solid cancer selected from breast cancer, lung cancer, thyroid cancer, and uterine cancer. The subject may be suffering from leptomeningeal disease. The subject may be suffering from neoplastic meningitis, carcinomatous meningitis, lymphomatous meningitis, or leukemic meningitis.

In certain embodiments, the immunotherapy is a PD-1 inhibitor selected from pembrolizumab and nivolumab. In certain embodiments, the immunotherapy is a PD-L1 inhibitor selected from atezolizumab, avelumab, and durvalumab. In certain embodiments, the immunotherapy is a CTLA-4 inhibitor. The CTLA-4 inhibitor may be ipilimumab.

In certain embodiments, detecting expression or activity of a gene signature comprises single cell RNA sequencing (scRNA-Seq). The scRNA-Seq may comprise Seq-Well.

In another aspect, the present invention provides for a method of treating cancer comprising depleting T cells having a non-responder gene signature.

In another aspect, the present invention provides for a kit comprising reagents to detect at least one signature gene or polypeptide according to claim 9.

In certain embodiments, the gene signature is detected in at least two time points post-treatment. The signature may be detected in 2, 3, 4, 5 or more time points post treatment.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—Description of study.

FIG. 2—Seq-Well on CSF samples yields highly complex single-cell RNA-Seq data.

FIG. 3A-B—Analysis of single-cell data from CSF reveals distinct cell types/states.

FIG. 4—Tumor cells exhibit upregulation of antigen presentation post-treatment.

FIG. 5—Antigen presentation in tumor cells correlates with interferon response.

FIG. 6—T cells exhibit canonical, patient-specific changes in phenotype.

FIG. 7—T cells from long-surviving patients exhibit active interferon regulation.

FIG. 8—T cells from long-surviving patients exhibit exhaustion/PD1 attenuation (shown below, in heatmap and as a feature plot of signature derived from these genes).

FIG. 9—T cells from long-surviving patients exhibit exhaustion/PD1 attenuation (shown below, in heatmap and as a feature plot of signature derived from these genes).

FIG. 10A-B—CSF014 tumor post vs. pre-treatment correlation plots. A) CSF014.tumor.post.var.corr, B) CSF014.tumor.pre.var.corr.

FIG. 11A-B—CSF029 tumor post vs. pre-treatment correlation plots. A) CSF029.tumor.post.var.corr, B) CSF029.tumor.pre.var.corr.

FIG. 12A-B—CSF037 tumor post vs. pre-treatment correlation plots. A) CSF037.tumor.post.var.corr, B) CSF037.tumor.pre.var.corr.

FIG. 13A-B—CSF043 tumor post vs. pre-treatment correlation plots. A) CSF043.tumor.post.var.corr, B) CSF043.tumor.pre.var.corr.

FIG. 14A-D—CSF tumor bulk responder vs. non responder correlation plots. A) CSF.tumor.NR.post.tumor.var.corr, B) CSF.tumor.NR.pre.tumor.var.corr, C) CSF.tumor.R.post.tumor.var.corr, D) CSF.tumor.R.post.tumor.var.corr.

FIG. 15A-B—CSF T cell bulk treated vs. untreated correlation plots. A) CSF.tcell.post.var.corr, B) CSF.tcell.pre.var.corr.

FIG. 16A-B—CSF T cell bulk responder v non-responder correlation plots. A) CSF.tcell.NR.var.corr, B) CSF.tcell.R.var.corr.

FIG. 17A-B—CSF mac bulk treated v untreated correlation plots. A) CSF.mac.post.var.corr, B) CSF.mac.pre.var.corr.

FIG. 18A-B—CSF mac bulk responder v non-responder correlation plots. A) CSF.mac.NR.var.corr, B) CSF.mac.R.var.corr.

FIG. 19A-F—CSF comparisons between BM and LMD correlation plots. A) CSF.mac.bm.var.corr, B) CSF.mac.bm.var.corr, C) CSF.tcell.bm.var.corr, D) CSF.tcell.lmd.var.corr, E) CSF.tcell.lmd.var.corr, F) CSF.tcell.lmd.var.corr.

FIG. 20A-B—A) Schematic showing recovery of CSF samples from a patient, performing Seq-well on pre-treatment and post treatment samples. B) UMAP clustering of single cells shaded by patient (left) and by status (right). Immune cells are indicated by dashed circles and tumors are indicated by solid circles. Cells are shaded by pre- and post-treatment status.

FIG. 21—UMAP clustering of T cells by phenotype showing four clusters.

FIG. 22—UMAP clustering of T cells by patient.

FIG. 23—Heat map of differential expressed genes in the four T cell clusters.

FIG. 24—UMAP subclustering of adaptive immune cells.

FIG. 25—Graph showing cell composition of the four T cell clusters.

FIG. 26—UMAP clustering of CD8 T cells by patient and treatment.

FIG. 27—UMAP clustering of CD8 T cells by phenotype.

FIG. 28—Graph showing fraction of post-treatment and pre-treatment cells in the CD8 prolif. cluster.

FIG. 29—Heat map of differential expressed genes between treated and untreated CD8 T cell clusters.

FIG. 30—Graph showing enrichments in pathways between treated and untreated CD8 T cell clusters.

FIG. 31—Graph showing enrichments in pathways between treated and untreated CD8 T cells.

FIG. 32—Violin plots scoring the CD8 T cell clusters against curated published signatures.

FIG. 33—Heat map of differential expressed genes between CD8.postA (CD8.post1, CD8.exh^{hi}) and CD8.postB (CD8.post2, CD8.exh^{lo}).

FIG. 34—Graph showing the proportion of each CD8 T cell clusters in post-treatment samples at two time points for two patients.

FIG. 35—Projection of published signatures on UMAP clusters of CD8 T cells.

FIG. 36—UMAP clustering of innate immune cells by patient and treatment.

FIG. 37—UMAP clustering of innate immune cells by cell type and treatment.

FIG. 38—Heat map of differential expressed genes in innate immune cells.

FIG. 39—Graph showing fraction of innate immune cell populations pre and post-treatment.

FIG. 40—Violin plots scoring the innate immune cell populations pre and post-treatment against an interferon response signature.

FIG. 41—Violin plots scoring the innate immune cell populations pre and post-treatment against an antigen processing signature.

FIG. 42—Violin plots scoring the monocyte and macrophage populations pre and post-treatment against the M1 and M2 phenotype.

FIG. 43—Heat maps showing differential expressed M1 and M2-like genes in the monocyte and macrophage populations pre and post-treatment.

FIG. 44—Violin plots scoring proinflammatory/immune activating genes in monocytes pre and post-treatment.

FIG. 45—Violin plots scoring proinflammatory/immune activating genes in macrophages pre and post-treatment.

FIG. 46—Violin plots scoring anti-inflammatory/tissue-remodeling genes in monocytes pre and post-treatment.

FIG. 47—Violin plots scoring anti-inflammatory/tissue-remodeling genes in macrophages pre and post-treatment.

FIG. 48—Plot showing upregulated genes post-treatment in the classical dendritic cell (cDC) population.

FIG. 49—Plot comparing percentage of IDO1 expressing cDCs and proliferating CD8+ T cells post-treatment in patient samples.

FIG. 50A-J—A) UMAP clustering of tumor cells by patient. B) UMAP clustering of tumor cells by status. C) Graph showing per-patient average for interferon-γ response pre- and post-treatment. D) Violin plots showing combined single-cell scores for interferon-γ response pre- and post-treatment. E) UMAP clustering of tumor cells by patient of patients with matched sampling of the same TME over time. F) UMAP clustering of tumor cells by status of patients with matched sampling of the same TME over time. G) Violin plots showing interferon-γ response over time for the four patients. H) Dimensionality reduction in ranked windowed mean expression (WME) space using UMAP. I) Plot of WME rank for each single cell in P043 ordered by chromosome to perform inferred CNV profiling. J) Violin plots scoring interferon-γ response for each cluster at the three available time points and pie charts showing cluster proportion over time.

FIG. 51—Bar graph showing percentage of tumor and immune cells pre- and post-treatment.

FIG. 52—Dimension reduction analysis of the adaptive immune compartment.

FIG. 53—Differential expression between T cells of the same subset between responding and non-responding patients.

FIG. 54—tSNE plots showing the clustering of pre-treatment T cells.

FIG. 55—Analysis of the innate immune compartment between responding and non-responding patients.

FIG. 56A-B—Analysis of pre-treatment tumor cells suggests disruption of JAK/STAT pathway underlies response A) tSNE analysis of tumor cells from responders and non-responders. B) Heatmap showing differential coexpression between responders and non-responders.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R.I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited in this application are indicative of the level of skill in the art(s) to which the application pertains. All publications, published patent documents, and patent applications cited herein are hereby incorporated by reference to the same extent as though each individual publication, published patent document, or patent application was specifically and individually indicated as being incorporated by reference.

### Overview

Brain metastases are a pressing clinical challenge, whose study is complicated by their biological complexity and difficulty obtaining samples. Overcoming these issues requires: 1) methods for resolving the cell states and biomarkers that inform metastases; and, 2) extraction of relevant information from clinically-obtainable samples. Applicants describe a novel platform to perform single-cell RNA-Seq (scRNA-seq) on liquid biopsies—here, cerebrospinal fluid (CSF)—from patients with brain metastases. Applicants adapted Seq-Well, a low-input, high-throughput scRNA-Seq platform, to profile CSF temporally from patients with brain metastases (leptomeningeal disease—LMD); 2) analyzed these data to decipher the phenotypic evolution of metastases under therapy (e.g., immunotherapy); and 3) compared CSF and LMD tissue from the same-patient to identify cellular phenotypes and biomarkers relevant to detection and surveillance.

To directly profile cellular and molecular changes in the TME in response to CBIs, Applicants initiated a unique immunotherapy clinical trial in which low-input samples were collected longitudinally from the same tumor reservoir and profiled using Seq-Well, a high-throughput single-cell RNA-Seq (scRNA-Seq) method specialized for paucicellular patient-derived specimens. Specifically, Applicants performed a phase-II clinical trial to examine the efficacy of the immune CBI Pembrolizumab, which targets PD-1 on T cells, in breast cancer patients diagnosed with leptomeningeal disease (LMD; metastases to the meninges). LMD is a devastating late-stage feature of many solid tumors, associated with a median survival of 3 months. Here, by sampling cerebrospinal fluid (CSF)—the LMD TME—before and after treatment, and applying Seq-Well (FIG. 20), Applicants characterize the cellular composition of LMD, detail treatment-induced shifts in cellular abundance and phenotype across multiple patients, and directly chart responses to CBI within specific individuals. Overall, the results typify the evolution of the LMD microenvironment following Pembrolizumab treatment, detailing response heterogeneity among and between specific cellular populations, as well as demonstrating the utility of longitudinal patient samples of the immune microenvironment.

Specifically, Applicants describe the results of single-cell resolution profiling of cross-patient and patient-specific responses to checkpoint blockade inhibitor Pembrolizumab in LMD. The data characterize shifts in composition and phenotype, detailing changes in T cell exhaustion and macrophage polarization induced by immunotherapy profiled at single-cell resolution in the same patient microenvironment. These observations, taken together, detail a complex response to immunotherapy in the LMD microenvironment, which may have important clinical consequences for treatment of these patients. Further, Applicants demonstrate the efficacy of paired cohort analysis, utilizing data from a temporally resolved patient group to further characterize features identified in a larger patient group. These analyses and subsequent results survey dynamic cellular behaviors in LMD during immunotherapy and describe an investigative approach to explore them and contextualize their consequences.

Embodiments disclosed herein provide methods and compositions for detecting and modulating an immunotherapy resistance or responder gene signature in cancer. Embodiments disclosed herein also provide for diagnosing, prognosing, monitoring and treating tumors based on detection of an immunotherapy resistance gene signature. Specific embodiments provide for detecting the gene signatures in single rare or low quantity cells obtained from the extracellular fluid or compartments from a subject in need thereof.

### Use of Gene Signatures for Detection, Screening, Monitoring and Administering Treatment

In certain example embodiments, the sample where a signature is detected may be a sample of extracellular fluid. As used herein “extracellular fluid” comprises any fluid outside the cell. Examples include, but are not limited to, blood, plasma, cerebrospinal fluid, synovial fluid, interstitial fluids, transcellular fluids, lymph, aqueous humor in the eye, serous fluid, saliva, intestinal secretions (e.g. gastric juice, pancreatic juice), bone marrow or other tissue aspirate, pharyngeal secretions, mucous, perilymph and endolymph. A compartment may be a space within or without a particular tissue characterized as containing one or more extracellular fluids.

In certain embodiments, detecting the signature may predict cancer survival. As used herein the term “cancer-specific survival” refers to the percentage of patients with a specific type and stage of cancer who have not died from their cancer during a certain period of time after diagnosis. The period of time may be 1 year, 2 years, 5 years, etc., with 5 years being the time period most often used. Cancer-specific survival is also called disease-specific survival. In most cases, cancer-specific survival is based on causes of death listed in medical records.

As used herein the term “relative survival” refers to a method used to estimate cancer-specific survival that does not use information about the cause of death. It is the percentage of cancer patients who have survived for a certain period of time after diagnosis compared to people who do not have cancer.

As used herein the term “overall survival” refers to the percentage of people with a specific type and stage of cancer who have not died from any cause during a certain period of time after diagnosis.

As used herein the term “disease-free survival” refers to the percentage of patients who have no signs of cancer during a certain period of time after treatment. Other names for this statistic are recurrence-free or progression-free survival.

As used herein a “signature” may encompass any gene or genes, protein or proteins, or epigenetic element(s) whose expression profile or whose occurrence is associated with a specific cell type, subtype, or cell state of a specific cell type or subtype within a population of cells (e.g., immune evading tumor cells, immunotherapy resistant tumor cells, tumor infiltrating lymphocytes, macrophages). In certain embodiments, the expression of the immunotherapy resistant, T cell signature and/or macrophage signature is dependent on epigenetic modification of the genes or regulatory elements associated with the genes. Thus, in certain embodiments, use of signature genes includes epigenetic modifications that may be detected or modulated. For ease of discussion, when discussing gene expression, any of gene or genes, protein or proteins, or epigenetic element(s) may be substituted. As used herein, the terms “signature”, “expression profile”, or “expression program” may be used interchangeably. It is to be understood that also when referring to proteins (e.g. differentially expressed proteins), such may fall within the definition of “gene” signature. Levels of expression or activity may be compared between different cells in order to characterize or identify for instance signatures specific for cell (sub)populations. Increased or decreased expression or activity or prevalence of signature genes may be compared between different cells in order to characterize or identify for instance specific cell (sub)populations. The detection of a signature in single cells may be used to identify and quantitate for instance specific cell (sub)populations. A signature may include a gene or genes, protein or proteins, or epigenetic element(s) whose expression or occurrence is specific to a cell (sub)population, such that expression or occurrence is exclusive to the cell (sub)population. A gene signature as used herein, may thus refer to any set of up- and/or down-regulated genes that are representative of a cell type or subtype. A gene signature as used herein, may also refer to any set of up- and/or down-regulated genes between different cells or cell (sub)populations derived from a gene-expression profile. For example, a gene signature may comprise a list of genes differentially expressed in a distinction of interest.

The signature as defined herein (being it a gene signature, protein signature or other genetic or epigenetic signature) can be used to indicate the presence of a cell type, a subtype of the cell type, the state of the microenvironment of a population of cells, a particular cell type population or subpopulation, and/or the overall status of the entire cell (sub)population. Furthermore, the signature may be indicative of cells within a population of cells in vivo. The signature may also be used to suggest for instance particular therapies, or to follow up treatment, or to suggest ways to modulate immune systems. The signatures of the present invention may be discovered by analysis of expression profiles of single-cells within a population of cells from isolated samples (e.g. tumor samples), thus allowing the discovery of novel cell subtypes or cell states that were previously invisible or unrecognized. The presence of subtypes or cell states may be determined by subtype specific or cell state specific signatures. The presence of these specific cell (sub)types or cell states may be determined by applying the signature genes to bulk sequencing data in a sample. Not being bound by a theory the signatures of the present invention may be microenvironment specific, such as their expression in a particular spatio-temporal context. Not being bound by a theory, signatures as discussed herein are specific to a particular pathological context. Not being bound by a theory, a combination of cell subtypes having a particular signature may indicate an outcome. Not being bound by a theory, the signatures can be used to deconvolute the network of cells present in a particular pathological condition. Not being bound by a theory the presence of specific cells and cell subtypes are indicative of a particular response to treatment, such as including increased or decreased susceptibility to treatment. The signature may indicate the presence of one particular cell type. In one embodiment, the novel signatures are used to detect multiple cell states or hierarchies that occur in subpopulations of cells that are linked to particular pathological condition, or linked to a particular outcome or progression of the disease, or linked to a particular response to treatment of the disease (e.g. resistance to immunotherapy).

The signature according to certain embodiments of the present invention may comprise or consist of one or more genes, proteins and/or epigenetic elements, such as for instance 1, 2, 3, 4, 5, 6, 7, 8, 9, 10 or more. In certain embodiments, the signature may comprise or consist of two or more genes, proteins and/or epigenetic elements, such as for instance 2, 3, 4, 5, 6, 7, 8, 9, 10 or more. In certain embodiments, the signature may comprise or consist of three or more genes, proteins and/or epigenetic elements, such as for instance 3, 4, 5, 6, 7, 8, 9, 10 or more. In certain embodiments, the signature may comprise or consist of four or more genes, proteins and/or epigenetic elements, such as for instance 4, 5, 6, 7, 8, 9, 10 or more. In certain embodiments, the signature may comprise or consist of five or more genes, proteins and/or epigenetic elements, such as for instance 5, 6, 7, 8, 9, 10 or more. In certain embodiments, the signature may comprise or consist of six or more genes, proteins and/or epigenetic elements, such as for instance 6, 7, 8, 9, 10 or more. In certain embodiments, the signature may comprise or consist of seven or more genes, proteins and/or epigenetic elements, such as for instance 7, 8, 9, 10 or more. In certain embodiments, the signature may comprise or consist of eight or more genes, proteins and/or epigenetic elements, such as for instance 8, 9, 10 or more. In certain embodiments, the signature may comprise or consist of nine or more genes, proteins and/or epigenetic elements, such as for instance 9, 10 or more. In certain embodiments, the signature may comprise or consist of ten or more genes, proteins and/or epigenetic elements, such as for instance 10, 11, 12, 13, 14, 15, or more. It is to be understood that a signature according to the invention may for instance also include genes or proteins as well as epigenetic elements combined.

In certain embodiments, a signature is characterized as being specific for a particular cell or cell (sub)population if it is upregulated or only present, detected or detectable in that particular cell or cell (sub)population, or alternatively is downregulated or only absent, or undetectable in that particular cell or cell (sub)population. In this context, a signature consists of one or more differentially expressed genes/proteins or differential epigenetic elements when comparing different cells or cell (sub)populations, including comparing different immune cells or immune cell (sub)populations (e.g., T cells), as well as comparing immune cells or immune cell (sub)populations with other immune cells or immune cell (sub)populations. It is to be understood that “differentially expressed” genes/proteins include genes/proteins which are up- or down-regulated as well as genes/proteins which are turned on or off. When referring to up- or down-regulation, in certain embodiments, such up- or down-regulation is preferably at least two-fold, such as two-fold, three-fold, four-fold, five-fold, or more, such as for instance at least ten-fold, at least 20-fold, at least 30-fold, at least 40-fold, at least 50-fold, or more. Alternatively, or in addition, differential expression may be determined based on common statistical tests, as is known in the art.

As discussed herein, differentially expressed genes/proteins, or differential epigenetic elements may be differentially expressed on a single cell level, or may be differentially expressed on a cell population level. Preferably, the differentially expressed genes/proteins or epigenetic elements as discussed herein, such as constituting the gene signatures as discussed herein, when as to the cell population level, refer to genes that are differentially expressed in all or substantially all cells of the population (such as at least 80%, preferably at least 90%, such as at least 95% of the individual cells). This allows one to define a particular subpopulation of cells. As referred to herein, a “subpopulation” of cells preferably refers to a particular subset of cells of a particular cell type (e.g., resistant) which can be distinguished or are uniquely identifiable and set apart from other cells of this cell type. The cell subpopulation may be phenotypically characterized, and is preferably characterized by the signature as discussed herein. A cell (sub)population as referred to herein may constitute of a (sub)population of cells of a particular cell type characterized by a specific cell state.

When referring to induction, or alternatively reducing or suppression of a particular signature, preferable is meant induction or alternatively reduction or suppression (or upregulation or downregulation) of at least one gene/protein and/or epigenetic element of the signature, such as for instance at least two, at least three, at least four, at least five, at least six, or all genes/proteins and/or epigenetic elements of the signature.

Various aspects and embodiments of the invention may involve analyzing gene signatures, protein signature, and/or other genetic or epigenetic signature based on single cell analyses (e.g. single cell RNA sequencing) or alternatively based on cell population analyses, as is defined herein elsewhere.

The invention further relates to various uses of the gene signatures, protein signature, and/or other genetic or epigenetic signature as defined herein, as well as various uses of the immune cells or immune cell (sub)populations as defined herein. Particular advantageous uses include methods for identifying agents capable of inducing or suppressing particular immune cell (sub)populations based on the gene signatures, protein signature, and/or other genetic or epigenetic signature as defined herein. The invention further relates to agents capable of inducing or suppressing particular immune cell (sub)populations based on the gene signatures, protein signature, and/or other genetic or epigenetic signature as defined herein, as well as their use for modulating, such as inducing or repressing, a particular gene signature, protein signature, and/or other genetic or epigenetic signature. In one embodiment, genes in one population of cells may be activated or suppressed in order to affect the cells of another population. In related aspects, modulating, such as inducing or repressing, a particular gene signature, protein signature, and/or other genetic or epigenetic signature may modify overall immune composition, such as immune cell composition, such as immune cell subpopulation composition or distribution, or functionality.

The signature genes of the present invention were discovered by analysis of expression profiles of single-cells within a population of tumor cells, thus allowing the discovery of novel cell subtypes that were previously invisible in a population of cells within a tumor. The presence of subtypes may be determined by subtype specific signature genes. The presence of these specific cell types may be determined by applying the signature genes to bulk sequencing data in a patient. Not being bound by a theory, many cells that make up a microenvironment, whereby the cells communicate and affect each other in specific ways. As such, specific cell types within this microenvironment may express signature genes specific for this microenvironment. Not being bound by a theory the signature genes of the present invention may be microenvironment specific, such as their expression in a tumor. The signature genes may indicate the presence of one particular cell type. In one embodiment, the expression may indicate the presence of immunotherapy resistant cell types. Not being bound by a theory, a combination of cell subtypes in a subject may indicate an outcome (e.g., resistant cells, cytotoxic T cells, Tregs). Exemplary gene signatures of the present invention are provided in Tables 1-10.

In certain embodiments, the present invention provides for gene signature screening. The concept of signature screening was introduced by Stegmaier et al. (Gene expression-based high-throughput screening (GE-HTS) and application to leukemia differentiation. Nature Genet. 36, 257-263 (2004)), who realized that if a gene-expression signature was the proxy for a phenotype of interest, it could be used to find small molecules that effect that phenotype without knowledge of a validated drug target. The signature of the present may be used to screen for drugs that reduce the signature in cancer cells or cell lines having a resistant signature as described herein. The signature may be used for GE-HTS. In certain embodiments, pharmacological screens may be used to identify drugs that are selectively toxic to cancer cells having an immunotherapy resistant signature. In certain embodiments, drugs selectively toxic to cancer cells having an immunotherapy resistant signature are used for treatment of a cancer patient. In certain embodiments, cells having an immunotherapy resistant signature as described herein are treated with a plurality of drug candidates not toxic to non-tumor cells and toxicity is assayed.

The Connectivity Map (cmap) is a collection of genome-wide transcriptional expression data from cultured human cells treated with bioactive small molecules and simple pattern-matching algorithms that together enable the discovery of functional connections between drugs, genes and diseases through the transitory feature of common gene-expression changes (see, Lamb et al., The Connectivity Map: Using Gene-Expression Signatures to Connect Small Molecules, Genes, and Disease. Science 29 Sep. 2006: Vol. 313, Issue 5795, pp. 1929-1935, DOI: 10.1126/science.1132939; and Lamb, J., The Connectivity Map: a new tool for biomedical research. Nature Reviews Cancer January 2007: Vol. 7, pp. 54-60). Cmap can be used to screen for a signature in silico.

In one embodiment, the signature genes may be detected by immunofluorescence, immunohistochemistry, fluorescence activated cell sorting (FACS), mass cytometry (CyTOF), mass spectrometry, RNA sequencing (e.g., RNA-seq), single cell RNA sequencing (e.g., Drop-seq, scRNA-seq, InDrop), single cell qPCR, MERFISH (multiplex (in situ) RNA FISH) and/or by in situ hybridization. Other methods including absorbance assays and colorimetric assays are known in the art and may be used herein.

In certain embodiments, the invention involves targeted nucleic acid profiling (e.g., sequencing, quantitative reverse transcription polymerase chain reaction, and the like) (see e.g., Geiss G K, et al., Direct multiplexed measurement of gene expression with color-coded probe pairs. Nat Biotechnol. 2008 March; 26(3):317-25). In certain embodiments, a target nucleic acid molecule (e.g., RNA molecule), may be sequenced by any method known in the art, for example, methods of high-throughput sequencing, also known as next generation sequencing or deep sequencing. A nucleic acid target molecule labeled with a barcode (for example, an origin-specific barcode) can be sequenced with the barcode to produce a single read and/or contig containing the sequence, or portions thereof, of both the target molecule and the barcode. Exemplary next generation sequencing technologies include, for example, Illumina sequencing, Ion Torrent sequencing, 454 sequencing, SOLiD sequencing, and nanopore sequencing amongst others. Methods for constructing sequencing libraries are known in the art (see, e.g., Head et al., Library construction for next-generation sequencing: Overviews and challenges. Biotechniques. 2014; 56(2): 61-77).

In certain embodiments, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain embodiments, the invention involves high-throughput single-cell RNA-seq and/or targeted nucleic acid profiling (for example, sequencing, quantitative reverse transcription polymerase chain reaction, and the like) where the RNAs from different cells are tagged individually, allowing a single library to be created while retaining the cell identity of each read. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; and Gierahn et al., “Seq-Well: portable, low-cost RNA sequencing of single cells at high throughput” Nature Methods 14, 395-398 (2017), all the contents and disclosure of each of which are herein incorporated by reference in their entirety. In preferred embodiments, Seq-well is performed to conserve valuable samples.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; and International patent application number PCT/US2016/059239, published as WO2017164936 on Sep. 28, 2017, which are herein incorporated by reference in their entirety.

In certain embodiments, the gene signatures described herein are screened by perturbation of target genes within said signatures. In certain embodiments, perturbation of any signature gene or gene described herein may reduce or induce the immunotherapy resistance signature. In certain embodiments, after perturbation, gene expression may be evaluated to determine whether the gene signature is reduced.

In certain embodiments, the gene signatures described herein are screened by perturbation of target genes within said signatures. Methods and tools for genome-scale screening of perturbations in single cells using CRISPR-Cas9 have been described, herein referred to as perturb-seq (see e.g., Dixit et al., “Perturb-Seq: Dissecting Molecular Circuits with Scalable Single-Cell RNA Profiling of Pooled Genetic Screens” 2016, Cell 167, 1853-1866; Adamson et al., “A Multiplexed Single-Cell CRISPR Screening Platform Enables Systematic Dissection of the Unfolded Protein Response” 2016, Cell 167, 1867-1882; Feldman et al., Lentiviral co-packaging mitigates the effects of intermolecular recombination and multiple integrations in pooled genetic screens, bioRxiv 262121, doi: doi.org/10.1101/262121; Datlinger, et al., 2017, Pooled CRISPR screening with single-cell transcriptome readout. Nature Methods. Vol. 14 No. 3 DOI: 10.1038/nmeth.4177; Hill et al., On the design of CRISPR-based single cell molecular screens, Nat Methods. 2018 April; 15(4): 271-274; and International publication serial number WO/2017/075294). The present invention is compatible with perturb-seq, such that signature genes may be perturbed and the perturbation may be identified and assigned to the proteomic and gene expression readouts of single cells. In certain embodiments, signature genes may be perturbed in single cells and gene expression analyzed. Not being bound by a theory, networks of genes that are disrupted due to perturbation of a signature gene may be determined. Understanding the network of genes effected by a perturbation may allow for a gene to be linked to a specific pathway that may be targeted to modulate the signature and treat a cancer. Thus, in certain embodiments, perturb-seq is used to discover novel drug targets to allow treatment of specific cancer patients having the gene signature of the present invention.

The perturbation methods and tools allow reconstructing of a cellular network or circuit. In one embodiment, the method comprises (1) introducing single-order or combinatorial perturbations to a population of cells, (2) measuring genomic, genetic, proteomic, epigenetic and/or phenotypic differences in single cells and (3) assigning a perturbation(s) to the single cells. Not being bound by a theory, a perturbation may be linked to a phenotypic change, preferably changes in gene or protein expression. In preferred embodiments, measured differences that are relevant to the perturbations are determined by applying a model accounting for co-variates to the measured differences. The model may include the capture rate of measured signals, whether the perturbation actually perturbed the cell (phenotypic impact), the presence of subpopulations of either different cells or cell states, and/or analysis of matched cells without any perturbation. In certain embodiments, the measuring of phenotypic differences and assigning a perturbation to a single cell is determined by performing single cell RNA sequencing (RNA-seq). In preferred embodiments, the single cell RNA-seq is performed by any method as described herein (e.g., Drop-seq, InDrop, 10× genomics). In certain embodiments, unique barcodes are used to perform Perturb-seq. In certain embodiments, a guide RNA is detected by RNA-seq using a transcript expressed from a vector encoding the guide RNA. The transcript may include a unique barcode specific to the guide RNA. Not being bound by a theory, a guide RNA and guide RNA barcode is expressed from the same vector and the barcode may be detected by RNA-seq. Not being bound by a theory, detection of a guide RNA barcode is more reliable than detecting a guide RNA sequence, reduces the chance of false guide RNA assignment and reduces the sequencing cost associated with executing these screens. Thus, a perturbation may be assigned to a single cell by detection of a guide RNA barcode in the cell. In certain embodiments, a cell barcode is added to the RNA in single cells, such that the RNA may be assigned to a single cell. Generating cell barcodes is described herein for single cell sequencing methods. In certain embodiments, a Unique Molecular Identifier (UMI) is added to each individual transcript and protein capture oligonucleotide. Not being bound by a theory, the UMI allows for determining the capture rate of measured signals, or preferably the binding events or the number of transcripts captured. Not being bound by a theory, the data is more significant if the signal observed is derived from more than one protein binding event or transcript. In preferred embodiments, Perturb-seq is performed using a guide RNA barcode expressed as a polyadenylated transcript, a cell barcode, and a UMI.

Perturb-seq combines emerging technologies in the field of genome engineering, single-cell analysis and immunology, in particular the CRISPR-Cas9 system and droplet single-cell sequencing analysis. In certain embodiments, a CRISPR system is used to create an INDEL at a target gene. In other embodiments, epigenetic screening is performed by applying CRISPRa/i/x technology (see, e.g., Konermann et al. “Genome-scale transcriptional activation by an engineered CRISPR-Cas9 complex” Nature. 2014 Dec. 10. doi: 10.1038/nature14136; Qi, L. S., et al. (2013). “Repurposing CRISPR as an RNA-guided platform for sequence-specific control of gene expression”. Cell. 152 (5): 1173-83; Gilbert, L. A., et al., (2013). “CRISPR-mediated modular RNA-guided regulation of transcription in eukaryotes”. Cell. 154 (2): 442-51; Komor et al, 2016, Programmable editing of a target base in genomic DNA without double-stranded DNA cleavage, Nature 533, 420-424; Nishida et al., 2016, Targeted nucleotide editing using hybrid prokaryotic and vertebrate adaptive immune systems, Science 353(6305); Yang et al., 2016, Engineering and optimising deaminase fusions for genome editing, Nat Commun. 7:13330; Hess et al., 2016, Directed evolution using dCas9-targeted somatic hypermutation in mammalian cells, Nature Methods 13, 1036-1042; and Ma et al., 2016, Targeted AID-mediated mutagenesis (TAM) enables efficient genomic diversification in mammalian cells, Nature Methods 13, 1029-1035). Numerous genetic variants associated with disease phenotypes are found to be in non-coding region of the genome, and frequently coincide with transcription factor (TF) binding sites and non-coding RNA genes. Not being bound by a theory, CRISPRa/i/x approaches may be used to achieve a more thorough and precise understanding of the implication of epigenetic regulation. In one embodiment, a CRISPR system may be used to activate gene transcription. A nuclease-dead RNA-guided DNA binding domain, dCas9, tethered to transcriptional repressor domains that promote epigenetic silencing (e.g., KRAB) may be used for “CRISPRi” that represses transcription. To use dCas9 as an activator (CRISPRa), a guide RNA is engineered to carry RNA binding motifs (e.g., MS2) that recruit effector domains fused to RNA-motif binding proteins, increasing transcription. A key dendritic cell molecule, p65, may be used as a signal amplifier, but is not required.

In certain embodiments, other CRISPR-based perturbations are readily compatible with Perturb-seq, including alternative editors such as CRISPR/Cpf1. In certain embodiments, Perturb-seq uses Cpf1 as the CRISPR enzyme for introducing perturbations. Not being bound by a theory, Cpf1 does not require Tracr RNA and is a smaller enzyme, thus allowing higher combinatorial perturbations to be tested.

The cell(s) may comprise a cell in a model non-human organism, a model non-human mammal that expresses a Cas protein, a mouse that expresses a Cas protein, a mouse that expresses Cpf1, a cell in vivo or a cell ex vivo or a cell in vitro (see e.g., WO 2014/093622 (PCT/US13/074667); US Patent Publication Nos. 20120017290 and 20110265198 assigned to Sangamo BioSciences, Inc.; US Patent Publication No. 20130236946 assigned to Cellectis; Platt et al., “CRISPR-Cas9 Knockin Mice for Genome Editing and Cancer Modeling” Cell (2014), 159(2): 440-455; “Oncogenic models based on delivery and use of the CRISPR-Cas systems, vectors and compositions” WO2014204723A1 “Delivery and use of the CRISPR-Cas systems, vectors and compositions for hepatic targeting and therapy” WO2014204726A1; “Delivery, use and therapeutic applications of the CRISPR-Cas systems and compositions for modeling mutations in leukocytes” WO2016049251; and Chen et al., “Genome-wide CRISPR Screen in a Mouse Model of Tumor Growth and Metastasis” 2015, Cell 160, 1246-1260). The cell(s) may also comprise a human cell. Mouse cell lines may include, but are not limited to neuro-2a cells and EL4 cell lines (ATCC TIB-39). Primary mouse T cells may be isolated from C57/BL6 mice. Primary mouse T cells may be isolated from Cas9-expressing mice.

In one embodiment, CRISPR/Cas9 may be used to perturb protein-coding genes or non-protein-coding DNA. CRISPR/Cas9 may be used to knockout protein-coding genes by frameshifts, point mutations, inserts, or deletions. An extensive toolbox may be used for efficient and specific CRISPR/Cas9 mediated knockout as described herein, including a double-nicking CRISPR to efficiently modify both alleles of a target gene or multiple target loci and a smaller Cas9 protein for delivery on smaller vectors (Ran, F. A., et al., In vivo genome editing using Staphylococcus aureus Cas9. Nature. 520, 186-191 (2015)). A genome-wide sgRNA mouse library (˜10 sgRNAs/gene) may also be used in a mouse that expresses a Cas9 protein (see, e.g., WO2014204727A1).

In one embodiment, perturbation is by deletion of regulatory elements. Non-coding elements may be targeted by using pairs of guide RNAs to delete regions of a defined size, and by tiling deletions covering sets of regions in pools.

In one embodiment, perturbation of genes is by RNAi. The RNAi may be shRNA's targeting genes. The shRNA's may be delivered by any methods known in the art. In one embodiment, the shRNA's may be delivered by a viral vector. The viral vector may be a lentivirus, adenovirus, or adeno associated virus (AAV).

A CRISPR system may be delivered to primary mouse T-cells. Over 80% transduction efficiency may be achieved with Lenti-CRISPR constructs in CD4 and CD8 T-cells. Despite success with lentiviral delivery, recent work by Hendel et al, (Nature Biotechnology 33, 985-989 (2015) doi:10.1038/nbt.3290) showed the efficiency of editing human T-cells with chemically modified RNA, and direct RNA delivery to T-cells via electroporation. In certain embodiments, perturbation in mouse primary T-cells may use these methods.

In certain embodiments, whole genome screens can be used for understanding the phenotypic readout of perturbing potential target genes. In preferred embodiments, perturbations target expressed genes as defined by a gene signature using a focused sgRNA library. Libraries may be focused on expressed genes in specific networks or pathways. In other preferred embodiments, regulatory drivers are perturbed. In certain embodiments, Applicants perform systematic perturbation of key genes that regulate T-cell function in a high-throughput fashion. In certain embodiments, Applicants perform systematic perturbation of key genes that regulate cancer cell function in a high-throughput fashion (e.g., immune resistance or immunotherapy resistance). Applicants can use gene expression profiling data to define the target of interest and perform follow-up single-cell and population RNA-seq analysis. Not being bound by a theory, this approach will accelerate the development of therapeutics for human disorders, in particular cancer. Not being bound by a theory, this approach will enhance the understanding of the biology of T-cells and tumor immunity, and accelerate the development of therapeutics for human disorders, in particular cancer, as described herein.

Not being bound by a theory, perturbation studies targeting the genes and gene signatures described herein could (1) generate new insights regarding regulation and interaction of molecules within the system that contribute to suppression of an immune response, such as in the case within the tumor microenvironment, and (2) establish potential therapeutic targets or pathways that could be translated into clinical application.

In certain embodiments, after determining Perturb-seq effects in cancer cells and/or primary T-cells, the cells are infused back to the tumor xenograft models (melanoma, such as B16F10 and colon cancer, such as CT26) to observe the phenotypic effects of genome editing. Not being bound by a theory, detailed characterization can be performed based on (1) the phenotypes related to tumor progression, tumor growth, immune response, etc. (2) the TILs that have been genetically perturbed by CRISPR-Cas9 can be isolated from tumor samples, subject to cytokine profiling, qPCR/RNA-seq, and single-cell analysis to understand the biological effects of perturbing the key driver genes within the tumor-immune cell contexts. Not being bound by a theory, this will lead to validation of TILs biology as well as lead to therapeutic targets.

All gene name symbols refer to the gene as commonly known in the art. The examples described herein refer to the human gene names and it is to be understood that the present invention also encompasses genes from other organisms (e.g., mouse genes). Gene symbols may be those referred to by the HUGO Gene Nomenclature Committee (HGNC) or National Center for Biotechnology Information (NCBI). Any reference to the gene symbol is a reference made to the entire gene or variants of the gene. The signature as described herein may encompass any of the genes described herein. In certain embodiments, the gene signature includes surface expressed and secreted proteins. Not being bound by a theory, surface proteins may be targeted for detection and isolation of cell types, or may be targeted therapeutically to modulate an immune response.

### Modulating Gene Signatures or Signature Genes

As used herein, “modulating” or “to modulate” generally means either reducing or inhibiting the expression or activity of, or alternatively increasing the expression or activity of a target gene. In particular, “modulating” or “to modulate” can mean either reducing or inhibiting the activity of, or alternatively increasing a (relevant or intended) biological activity of, a target or antigen as measured using a suitable in vitro, cellular or in vivo assay (which will usually depend on the target involved), by at least 5%, at least 10%, at least 25%, at least 50%, at least 60%, at least 70%, at least 80%, at least 90%, or more, compared to activity of the target in the same assay under the same conditions but without the presence of an agent. An “increase” or “decrease” refers to a statistically significant increase or decrease respectively. For the avoidance of doubt, an increase or decrease will be at least 10% relative to a reference, such as at least 10%, at least 20%, at least 30%, at least 40%, at least 50%,a t least 60%, at least 70%, at least 80%, at least 90%, at least 95%, at least 97%, at least 98%, or more, up to and including at least 100% or more, in the case of an increase, for example, at least 2-fold, at least 3-fold, at least 4-fold, at least 5-fold, at least 6-fold, at least 7-fold, at least 8-fold, at least 9-fold, at least 10-fold, at least 50-fold, at least 100-fold, or more. “Modulating” can also involve effecting a change (which can either be an increase or a decrease) in affinity, avidity, specificity and/or selectivity of a target or antigen, such as a receptor and ligand. “Modulating” can also mean effecting a change with respect to one or more biological or physiological mechanisms, effects, responses, functions, pathways or activities in which the target or antigen (or in which its substrate(s), ligand(s) or pathway(s) are involved, such as its signaling pathway or metabolic pathway and their associated biological or physiological effects) is involved. Again, as will be clear to the skilled person, such an action as an agonist or an antagonist can be determined in any suitable manner and/or using any suitable assay known or described herein (e.g., in vitro or cellular assay), depending on the target or antigen involved.

Modulating can, for example, also involve allosteric modulation of the target and/or reducing or inhibiting the binding of the target to one of its substrates or ligands and/or competing with a natural ligand, substrate for binding to the target. Modulating can also involve activating the target or the mechanism or pathway in which it is involved. Modulating can for example also involve effecting a change in respect of the folding or confirmation of the target, or in respect of the ability of the target to fold, to change its conformation (for example, upon binding of a ligand), to associate with other (sub)units, or to disassociate. Modulating can for example also involve effecting a change in the ability of the target to signal, phosphorylate, dephosphorylate, and the like.

As used herein, an “agent” can refer to a protein-binding agent that permits modulation of activity of proteins or disrupts interactions of proteins and other biomolecules, such as but not limited to disrupting protein-protein interaction, ligand-receptor interaction, or protein-nucleic acid interaction. Agents can also refer to DNA targeting or RNA targeting agents. Agents may include a fragment, derivative and analog of an active agent. The terms “fragment,” “derivative” and “analog” when referring to polypeptides as used herein refers to polypeptides which either retain substantially the same biological function or activity as such polypeptides. An analog includes a proprotein which can be activated by cleavage of the proprotein portion to produce an active mature polypeptide. Such agents include, but are not limited to, antibodies (“antibodies” includes antigen-binding portions of antibodies such as epitope- or antigen-binding peptides, paratopes, functional CDRs; recombinant antibodies; chimeric antibodies; humanized antibodies; nanobodies; tribodies; midibodies; or antigen-binding derivatives, analogs, variants, portions, or fragments thereof), protein-binding agents, nucleic acid molecules, small molecules, recombinant protein, peptides, aptamers, avimers and protein-binding derivatives, portions or fragments thereof. An “agent” as used herein, may also refer to an agent that inhibits expression of a gene, such as but not limited to a DNA targeting agent (e.g., CRISPR system, TALE, Zinc finger protein) or RNA targeting agent (e.g., inhibitory nucleic acid molecules such as RNAi, miRNA, ribozyme).

The agents of the present invention may be modified, such that they acquire advantageous properties for therapeutic use (e.g., stability and specificity), but maintain their biological activity.

It is well known that the properties of certain proteins can be modulated by attachment of polyethylene glycol (PEG) polymers, which increases the hydrodynamic volume of the protein and thereby slows its clearance by kidney filtration. (See, e.g., Clark et al., J. Biol. Chem. 271: 21969-21977 (1996)). Therefore, it is envisioned that certain agents can be PEGylated (e.g., on peptide residues) to provide enhanced therapeutic benefits such as, for example, increased efficacy by extending half-life in vivo. In certain embodiments, PEGylation of the agents may be used to extend the serum half-life of the agents and allow for particular agents to be capable of crossing the blood-brain barrier.

In regards to peptide PEGylation methods, reference is made to Lu et al., Int. J. Pept. Protein Res. 43: 127-38 (1994); Lu et al., Pept. Res. 6: 140-6 (1993); Felix et al., Int. J. Pept. Protein Res. 46: 253-64 (1995); Gaertner et al., Bioconjug. Chem. 7: 38-44 (1996); Tsutsumi et al., Thromb. Haemost. 77: 168-73 (1997); Francis et al., hit. J. Hematol. 68: 1-18 (1998); Roberts et al., J. Pharm. Sci. 87: 1440-45 (1998); and Tan et al., Protein Expr. Purif 12: 45-52 (1998). Polyethylene glycol or PEG is meant to encompass any of the forms of PEG that have been used to derivatize other proteins, including, but not limited to, mono-(C1-10) alkoxy or aryloxy-polyethylene glycol. Suitable PEG moieties include, for example, 40 kDa methoxy poly(ethylene glycol) propionaldehyde (Dow, Midland, Mich.); 60 kDa methoxy poly(ethylene glycol) propionaldehyde (Dow, Midland, Mich.); 40 kDa methoxy poly(ethylene glycol) maleimido-propionamide (Dow, Midland, Mich.); 31 kDa alpha-methyl-w-(3-oxopropoxy), polyoxyethylene (NOF Corporation, Tokyo); mPEG2-NHS-40k (Nektar); mPEG2-MAL-40k (Nektar), SUNBRIGHT GL2-400MA ((PEG)240 kDa) (NOF Corporation, Tokyo), SUNBRIGHT ME-200MA (PEG20 kDa) (NOF Corporation, Tokyo). The PEG groups are generally attached to the peptide (e.g., neuromedin U receptor agonists or antagonists) via acylation or alkylation through a reactive group on the PEG moiety (for example, a maleimide, an aldehyde, amino, thiol, or ester group) to a reactive group on the peptide (for example, an aldehyde, amino, thiol, a maleimide, or ester group).

The PEG molecule(s) may be covalently attached to any Lys, Cys, or K(CO(CH2)2SH) residues at any position in a peptide. In certain embodiments, the neuromedin U receptor agonists described herein can be PEGylated directly to any amino acid at the N-terminus by way of the N-terminal amino group. A “linker arm” may be added to a peptide to facilitate PEGylation. PEGylation at the thiol side-chain of cysteine has been widely reported (see, e.g., Caliceti & Veronese, Adv. Drug Deliv. Rev. 55: 1261-77 (2003)). If there is no cysteine residue in the peptide, a cysteine residue can be introduced through substitution or by adding a cysteine to the N-terminal amino acid.

Substitutions of amino acids may be used to modify an agent of the present invention. The phrase “substitution of amino acids” as used herein encompasses substitution of amino acids that are the result of both conservative and non-conservative substitutions. Conservative substitutions are the replacement of an amino acid residue by another similar residue in a polypeptide. Typical but not limiting conservative substitutions are the replacements, for one another, among the aliphatic amino acids Ala, Val, Leu and Ile; interchange of Ser and Thr containing hydroxy residues, interchange of the acidic residues Asp and Glu, interchange between the amide-containing residues Asn and Gln, interchange of the basic residues Lys and Arg, interchange of the aromatic residues Phe and Tyr, and interchange of the small-sized amino acids Ala, Ser, Thr, Met, and Gly. Non-conservative substitutions are the replacement, in a polypeptide, of an amino acid residue by another residue which is not biologically similar. For example, the replacement of an amino acid residue with another residue that has a substantially different charge, a substantially different hydrophobicity, or a substantially different spatial configuration.

The term “antibody” is used interchangeably with the term “immunoglobulin” herein, and includes intact antibodies, fragments of antibodies, e.g., Fab, F(ab′)2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc, Fd, dAb, V_{HH} and scFv and/or Fv fragments.

As used herein, a preparation of antibody protein having less than about 50% of non-antibody protein (also referred to herein as a “contaminating protein”), or of chemical precursors, is considered to be “substantially free.” 40%, 30%, 20%, 10% and more preferably 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

The term “antigen-binding fragment” refers to a polypeptide fragment of an immunoglobulin or antibody that binds antigen or competes with intact antibody (i.e., with the intact antibody from which they were derived) for antigen binding (i.e., specific binding). As such these antibodies or fragments thereof are included in the scope of the invention, provided that the antibody or fragment binds specifically to a target molecule.

It is intended that the term “antibody” encompass any Ig class or any Ig subclass (e.g. the IgG1, IgG2, IgG3, and IgG4 subclassess of IgG) obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG1, IgG2, IgG3, and IgG4) that have been identified in humans and higher mammals. The antibodies can exist in monomeric or polymeric form; for example, IgM antibodies exist in pentameric form, and IgA antibodies exist in monomeric, dimeric or multimeric form.

The term “IgG subclass” refers to the four subclasses of immunoglobulin class IgG-IgG1, IgG2, IgG3, and IgG4 that have been identified in humans and higher mammals by the heavy chains of the immunoglobulins, V1-γ4, respectively. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by p pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains).

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

The term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat).

Such scaffolds have been extensively reviewed in Binz et al. (Engineering novel binding proteins from nonimmunoglobulin domains. Nat Biotechnol 2005, 23:1257-1268), Gebauer and Skerra (Engineered protein scaffolds as next-generation antibody therapeutics. Curr Opin Chem Biol. 2009, 13:245-55), Gill and Damle (Biopharmaceutical drug discovery using novel protein scaffolds. Curr Opin Biotechnol 2006, 17:653-658), Skerra (Engineered protein scaffolds for molecular recognition. J Mol Recognit 2000, 13:167-187), and Skerra (Alternative non-antibody scaffolds for molecular recognition. Curr Opin Biotechnol 2007, 18:295-304), and include without limitation affibodies, based on the Z-domain of staphylococcal protein A, a three-helix bundle of 58 residues providing an interface on two of its alpha-helices (Nygren, Alternative binding proteins: Affibody binding proteins developed from a small three-helix bundle scaffold. FEBS J 2008, 275:2668-2676); engineered Kunitz domains based on a small (ca. 58 residues) and robust, disulphide-crosslinked serine protease inhibitor, typically of human origin (e.g. LACI-D1), which can be engineered for different protease specificities (Nixon and Wood, Engineered protein inhibitors of proteases. Curr Opin Drug Discov Dev 2006, 9:261-268); monobodies or adnectins based on the 10th extracellular domain of human fibronectin III (10Fn3), which adopts an Ig-like beta-sandwich fold (94 residues) with 2-3 exposed loops, but lacks the central disulphide bridge (Koide and Koide, Monobodies: antibody mimics based on the scaffold of the fibronectin type III domain. Methods Mol Biol 2007, 352:95-109); anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms (Skerra, Alternative binding proteins: Anticalins—harnessing the structural plasticity of the lipocalin ligand pocket to engineer novel binding activities. FEBS J 2008, 275:2677-2683); DARPins, designed ankyrin repeat domains (166 residues), which provide a rigid interface arising from typically three repeated beta-turns (Stumpp et al., DARPins: a new generation of protein therapeutics. Drug Discov Today 2008, 13:695-701); avimers (multimerized LDLR-A module) (Silverman et al., Multivalent avimer proteins evolved by exon shuffling of a family of human receptor domains. Nat Biotechnol 2005, 23:1556-1561); and cysteine-rich knottin peptides (Kolmar, Alternative binding proteins: biological activity and therapeutic potential of cystine-knot miniproteins. FEBS J 2008, 275:2684-2690).

“Specific binding” of an antibody means that the antibody exhibits appreciable affinity for a particular antigen or epitope and, generally, does not exhibit significant cross reactivity. “Appreciable” binding includes binding with an affinity of at least 25 μM. Antibodies with affinities greater than 1×10⁷ M⁻¹ (or a dissociation coefficient of 1 M or less or a dissociation coefficient of 1 nm or less) typically bind with correspondingly greater specificity. Values intermediate of those set forth herein are also intended to be within the scope of the present invention and antibodies of the invention bind with a range of affinities, for example, 100 nM or less, 75 nM or less, 50 nM or less, 25 nM or less, for example 10 nM or less, 5 nM or less, 1 nM or less, or in embodiments 500 pM or less, 100 pM or less, 50 pM or less or 25 pM or less. An antibody that “does not exhibit significant crossreactivity” is one that will not appreciably bind to an entity other than its target (e.g., a different epitope or a different molecule). For example, an antibody that specifically binds to a target molecule will appreciably bind the target molecule but will not significantly react with non-target molecules or peptides. An antibody specific for a particular epitope will, for example, not significantly crossreact with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The dissociation constant, Kd, and the association constant, Ka, are quantitative measures of affinity.

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity but which recognize a common antigen. Monoclonal and polyclonal antibodies may exist within bodily fluids, as crude preparations, or may be purified, as described herein.

The term “binding portion” of an antibody (or “antibody portion”) includes one or more complete domains, e.g., a pair of complete domains, as well as fragments of an antibody that retain the ability to specifically bind to a target molecule. It has been shown that the binding function of an antibody can be performed by fragments of a full-length antibody. Binding fragments are produced by recombinant DNA techniques, or by enzymatic or chemical cleavage of intact immunoglobulins. Binding fragments include Fab, Fab′, F(ab′)2, Fabc, Fd, dAb, Fv, single chains, single-chain antibodies, e.g., scFv, and single domain antibodies.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

Examples of portions of antibodies or epitope-binding proteins encompassed by the present definition include: (i) the Fab fragment, having V_{L}, C_{L}, V_{H} and C_{H}1 domains; (ii) the Fab′ fragment, which is a Fab fragment having one or more cysteine residues at the C-terminus of the C_{H}1 domain; (iii) the Fd fragment having V_{H} and C_{H}1 domains; (iv) the Fd′ fragment having V_{H} and C_{H}1 domains and one or more cysteine residues at the C-terminus of the CHI domain; (v) the Fv fragment having the V_{L} and V_{H} domains of a single arm of an antibody; (vi) the dAb fragment (Ward et al., 341 Nature 544 (1989)) which consists of a V_{H} domain or a V_{L} domain that binds antigen; (vii) isolated CDR regions or isolated CDR regions presented in a functional framework; (viii) F(ab′)₂ fragments which are bivalent fragments including two Fab′ fragments linked by a disulphide bridge at the hinge region; (ix) single chain antibody molecules (e.g., single chain Fv; scFv) (Bird et al., 242 Science 423 (1988); and Huston et al., 85 PNAS 5879 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; Hollinger et al., 90 PNAS 6444 (1993)); (xi) “linear antibodies” comprising a pair of tandem Fd segments (V_{H}—Cₕ1-V_{H}-Cₕ1) which, together with complementary light chain polypeptides, form a pair of antigen binding regions (Zapata et al., Protein Eng. 8(10):1057-62 (1995); and U.S. Pat. No. 5,641,870).

As used herein, a “blocking” antibody or an antibody “antagonist” is one which inhibits or reduces biological activity of the antigen(s) it binds. In certain embodiments, the blocking antibodies or antagonist antibodies or portions thereof described herein completely inhibit the biological activity of the antigen(s).

Antibodies may act as agonists or antagonists of the recognized polypeptides. For example, the present invention includes antibodies which disrupt receptor/ligand interactions either partially or fully. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis. In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex. Likewise, encompassed by the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides disclosed herein. The antibody agonists and antagonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. III (Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(1):14-20 (1996).

The antibodies as defined for the present invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Simple binding assays can be used to screen for or detect agents that bind to a target protein, or disrupt the interaction between proteins (e.g., a receptor and a ligand). Because certain targets of the present invention are transmembrane proteins, assays that use the soluble forms of these proteins rather than full-length protein can be used, in some embodiments. Soluble forms include, for example, those lacking the transmembrane domain and/or those comprising the IgV domain or fragments thereof which retain their ability to bind their cognate binding partners. Further, agents that inhibit or enhance protein interactions for use in the compositions and methods described herein, can include recombinant peptido-mimetics.

Detection methods useful in screening assays include antibody-based methods, detection of a reporter moiety, detection of cytokines as described herein, and detection of a gene signature as described herein.

Another variation of assays to determine binding of a receptor protein to a ligand protein is through the use of affinity biosensor methods. Such methods may be based on the piezoelectric effect, electrochemistry, or optical methods, such as ellipsometry, optical wave guidance, and surface plasmon resonance (SPR).

The disclosure also encompasses nucleic acid molecules, in particular those that inhibit a signature gene. Exemplary nucleic acid molecules include aptamers, siRNA, artificial microRNA, interfering RNA or RNAi, dsRNA, ribozymes, antisense oligonucleotides, and DNA expression cassettes encoding said nucleic acid molecules. Preferably, the nucleic acid molecule is an antisense oligonucleotide. Antisense oligonucleotides (ASO) generally inhibit their target by binding target mRNA and sterically blocking expression by obstructing the ribosome. ASOs can also inhibit their target by binding target mRNA thus forming a DNA-RNA hybrid that can be a substance for RNase H. Preferred ASOs include Locked Nucleic Acid (LNA), Peptide Nucleic Acid (PNA), and morpholinos Preferably, the nucleic acid molecule is an RNAi molecule, i.e., RNA interference molecule. Preferred RNAi molecules include siRNA, shRNA, and artificial miRNA. The design and production of siRNA molecules is well known to one of skill in the art (e.g., Hajeri P B, Singh S K. Drug Discov Today. 2009 14(17-18):851-8). The nucleic acid molecule inhibitors may be chemically synthesized and provided directly to cells of interest. The nucleic acid compound may be provided to a cell as part of a gene delivery vehicle. Such a vehicle is preferably a liposome or a viral gene delivery vehicle.

### Adoptive Cell Therapy

In certain embodiments, tumor cells are targeted by using adoptive cell therapy (e.g., targeting resistant tumor cells). In certain embodiments, adoptive cell therapy is used in a combination treatment. In certain embodiments, a resistant signature is reduced before adoptive cell transfer. As used herein, “ACT”, “adoptive cell therapy” and “adoptive cell transfer” may be used interchangeably. In certain embodiments, Adoptive cell therapy (ACT) can refer to the transfer of cells to a patient with the goal of transferring the functionality and characteristics into the new host by engraftment of the cells (see, e.g., Mettananda et al., Editing an α-globin enhancer in primary human hematopoietic stem cells as a treatment for β-thalassemia, Nat Commun. 2017 Sep. 4; 8(1):424). As used herein, the term “engraft” or “engraftment” refers to the process of cell incorporation into a tissue of interest in vivo through contact with existing cells of the tissue. Adoptive cell therapy (ACT) can refer to the transfer of cells, most commonly immune-derived cells, back into the same patient or into a new recipient host with the goal of transferring the immunologic functionality and characteristics into the new host. If possible, use of autologous cells helps the recipient by minimizing GVHD issues. The adoptive transfer of autologous tumor infiltrating lymphocytes (TIL) (Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57.) or genetically re-directed peripheral blood mononuclear cells (Johnson et al., (2009) Blood 114 (3): 535-46; and Morgan et al., (2006) Science 314(5796) 126-9) has been used to successfully treat patients with advanced solid tumors, including melanoma and colorectal carcinoma, as well as patients with CD19-expressing hematologic malignancies (Kalos et al., (2011) Science Translational Medicine 3 (95): 95ra73). In certain embodiments, allogenic cells immune cells are transferred (see, e.g., Ren et al., (2017) Clin Cancer Res 23 (9) 2255-2266). As described further herein, allogenic cells can be edited to reduce alloreactivity and prevent graft-versus-host disease. Thus, use of allogenic cells allows for cells to be obtained from healthy donors and prepared for use in patients as opposed to preparing autologous cells from a patient after diagnosis.

Aspects of the invention involve the adoptive transfer of immune system cells, such as T cells, specific for selected antigens, such as tumor associated antigens or tumor specific neoantigens (see, e.g., Maus et al., 2014, Adoptive Immunotherapy for Cancer or Viruses, Annual Review of Immunology, Vol. 32: 189-225; Rosenberg and Restifo, 2015, Adoptive cell transfer as personalized immunotherapy for human cancer, Science Vol. 348 no. 6230 pp. 62-68; Restifo et al., 2015, Adoptive immunotherapy for cancer: harnessing the T cell response. Nat. Rev. Immunol. 12(4): 269-281; and Jenson and Riddell, 2014, Design and implementation of adoptive therapy with chimeric antigen receptor-modified T cells. Immunol Rev. 257(1): 127-144; and Rajasagi et al., 2014, Systematic identification of personal tumor-specific neoantigens in chronic lymphocytic leukemia. Blood. 2014 Jul. 17; 124(3):453-62).

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of: B cell maturation antigen (BCMA) (see, e.g., Friedman et al., Effective Targeting of Multiple BCMA-Expressing Hematological Malignancies by Anti-BCMA CAR T Cells, Hum Gene Ther. 2018 Mar. 8; Berdeja J G, et al. Durable clinical responses in heavily pretreated patients with relapsed/refractory multiple myeloma: updated results from a multicenter study of bb2121 anti-Bcma CAR T cell therapy. Blood. 2017; 130:740; and Mouhieddine and Ghobrial, Immunotherapy in Multiple Myeloma: The Era of CAR T Cell Therapy, Hematologist, May-June 2018, Volume 15, issue 3); PSA (prostate-specific antigen); prostate-specific membrane antigen (PSMA); PSCA (Prostate stem cell antigen); Tyrosine-protein kinase transmembrane receptor ROR1; fibroblast activation protein (FAP); Tumor-associated glycoprotein 72 (TAG72); Carcinoembryonic antigen (CEA); Epithelial cell adhesion molecule (EPCAM); Mesothelin; Human Epidermal growth factor Receptor 2 (ERBB2 (Her2/neu)); Prostase; Prostatic acid phosphatase (PAP); elongation factor 2 mutant (ELF2M); Insulin-like growth factor 1 receptor (IGF-1R); gplOO; BCR-ABL (breakpoint cluster region-Abelson); tyrosinase; New York esophageal squamous cell carcinoma 1 (NY-ESO-1); x-light chain, LAGE (L antigen); MAGE (melanoma antigen); Melanoma-associated antigen 1 (MAGE-A1); MAGE A3; MAGE A6; legumain; Human papillomavirus (HPV) E6; HPV E7; prostein; survivin; PCTA1 (Galectin 8); Melan-A/MART-1; Ras mutant; TRP-1 (tyrosinase related protein 1, or gp75); Tyrosinase-related Protein 2 (TRP2); TRP-2/INT2 (TRP-2/intron 2); RAGE (renal antigen); receptor for advanced glycation end products 1 (RAGEl); Renal ubiquitous 1, 2 (RU1, RU2); intestinal carboxyl esterase (iCE); Heat shock protein 70-2 (HSP70-2) mutant; thyroid stimulating hormone receptor (TSHR); CD123; CD171; CD19; CD20; CD22; CD26; CD30; CD33; CD44v7/8 (cluster of differentiation 44, exons 7/8); CD53; CD92; CD100; CD148; CD150; CD200; CD261; CD262; CD362; CS-1 (CD2 subset 1, CRACC, SLAMF7, CD319, and 19A24); C-type lectin-like molecule-1 (CLL-1); ganglioside GD3 (aNeu5Ac(2-8)aNeu5Ac(2-3)bDGalp(1-4)bDGlcp(1-1)Cer); Tn antigen (Tn Ag); Fms-Like Tyrosine Kinase 3 (FLT3); CD38; CD138; CD44v6; B7H3 (CD276); KIT (CD117); Interleukin-13 receptor subunit alpha-2 (IL-13Ra2); Interleukin 11 receptor alpha (IL-11Ra); prostate stem cell antigen (PSCA); Protease Serine 21 (PRSS21); vascular endothelial growth factor receptor 2 (VEGFR2); Lewis(Y) antigen; CD24; Platelet-derived growth factor receptor beta (PDGFR-beta); stage-specific embryonic antigen-4 (SSEA-4); Mucin 1, cell surface associated (MUC1); mucin 16 (MUC16); epidermal growth factor receptor (EGFR); epidermal growth factor receptor variant III (EGFRvIII); neural cell adhesion molecule (NCAM); carbonic anhydrase IX (CAIX); Proteasome (Prosome, Macropain) Subunit, Beta Type, 9 (LMP2); ephrin type-A receptor 2 (EphA2); Ephrin B2; Fucosyl GM1; sialyl Lewis adhesion molecule (sLe); ganglioside GM3 (aNeu5Ac(2-3)bDGalp(1-4)bDGlcp(1-1)Cer); TGS5; high molecular weight-melanoma-associated antigen (HMWMAA); β-acetyl-GD2 ganglioside (OAcGD2); Folate receptor alpha; Folate receptor beta; tumor endothelial marker 1 (TEM1/CD248); tumor endothelial marker 7-related (TEM7R); claudin 6 (CLDN6); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); ETS translocation-variant gene 6, located on chromosome 12p (ETV6-AML); sperm protein 17 (SPA17); X Antigen Family, Member 1A (XAGEl); angiopoietin-binding cell surface receptor 2 (Tie 2); CT (cancer/testis (antigen)); melanoma cancer testis antigen-1 (MAD-CT-1); melanoma cancer testis antigen-2 (MAD-CT-2); Fos-related antigen 1; p5³; p⁵³ mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; Cyclin D1; v-myc avian myelocytomatosis viral oncogene neuroblastoma derived homolog (MYCN); Ras Homolog Family Member C (RhoC); Cytochrome P450 1B1 (CYP1B1); CCCTC-Binding Factor (Zinc Finger Protein)-Like (BORIS); Squamous Cell Carcinoma Antigen Recognized By T Cells-1 or 3 (SART1, SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint-1, -2, -3 or -4 (SSX1, SSX2, SSX3, SSX4); CD79a; CD79b; CD72; Leukocyte-associated immunoglobulin-like receptor 1 (LAIR1); Fc fragment of IgA receptor (FCAR); Leukocyte immunoglobulin-like receptor subfamily A member 2 (LILRA2); CD300 molecule-like family member f (CD300LF); C-type lectin domain family 12 member A (CLECi2A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); mouse double minute 2 homolog (MDM2); livin; alphafetoprotein (AFP); transmembrane activator and CAML Interactor (TACI); B-cell activating factor receptor (BAFF-R); V-Ki-ras2 Kirsten rat sarcoma viral oncogene homolog (KRAS); immunoglobulin lambda-like polypeptide 1 (IGLL1); 707-AP (707 alanine proline); ART-4 (adenocarcinoma antigen recognized by T4 cells); BAGE (B antigen; b-catenin/m, b-catenin/mutated); CAMEL (CTL-recognized antigen on melanoma); CAPi (carcinoembryonic antigen peptide 1); CASP-8 (caspase-8); CDC27m (cell-division cycle 27 mutated); CDK4/m (cycline-dependent kinase 4 mutated); Cyp-B (cyclophilin B); DAM (differentiation antigen melanoma); EGP-2 (epithelial glycoprotein 2); EGP-40 (epithelial glycoprotein 40); Erbb2, 3, 4 (erythroblastic leukemia viral oncogene homolog-2, -3, 4); FBP (folate binding protein); fAchR (Fetal acetylcholine receptor); G250 (glycoprotein 250); GAGE (G antigen); GnT-V (N-acetylglucosaminyltransferase V); HAGE (helicose antigen); ULA-A (human leukocyte antigen-A); HST2 (human signet ring tumor 2); KIAA0205; KDR (kinase insert domain receptor); LDLR/FUT (low density lipid receptor/GDP L-fucose: b-D-galactosidase 2-a-L fucosyltransferase); L1CAM (L1 cell adhesion molecule); MC1R (melanocortin 1 receptor); Myosin/m (myosin mutated); MUM-1, -2, -3 (melanoma ubiquitous mutated 1, 2, 3); NA88-A (NA cDNA clone of patient M88); KG2D (Natural killer group 2, member D) ligands; oncofetal antigen (h5T4); p190 minor ber-abl (protein of 190 KD ber-abl); Pml/RARa (promyelocytic leukaemia/retinoic acid receptor a); PRAME (preferentially expressed antigen of melanoma); SAGE (sarcoma antigen); TEL/AML1 (translocation Ets-family leukemia/acute myeloid leukemia 1); TPI/m (triosephosphate isomerase mutated); CD70; and any combination thereof.

In certain embodiments, an antigen to be targeted in adoptive cell therapy (such as particularly CAR or TCR T-cell therapy) of a disease (such as particularly of tumor or cancer) is a tumor-specific antigen (TSA).

In certain embodiments, an antigen to be targeted in adoptive cell therapy (such as particularly CAR or TCR T-cell therapy) of a disease (such as particularly of tumor or cancer) is a neoantigen.

In certain embodiments, an antigen to be targeted in adoptive cell therapy (such as particularly CAR or TCR T-cell therapy) of a disease (such as particularly of tumor or cancer) is a tumor-associated antigen (TAA).

In certain embodiments, an antigen to be targeted in adoptive cell therapy (such as particularly CAR or TCR T-cell therapy) of a disease (such as particularly of tumor or cancer) is a universal tumor antigen. In certain preferred embodiments, the universal tumor antigen is selected from the group consisting of: a human telomerase reverse transcriptase (hTERT), survivin, mouse double minute 2 homolog (MDM2), cytochrome P450 1B 1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (D1), and any combinations thereof.

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of: CD19, BCMA, CD70, CLL-1, MAGE A3, MAGE A6, HPV E6, HPV E7, WT1, CD22, CD171, ROR1, MUC16, and SSX2. In certain preferred embodiments, the antigen may be CD19. For example, CD19 may be targeted in hematologic malignancies, such as in lymphomas, more particularly in B-cell lymphomas, such as without limitation in diffuse large B-cell lymphoma, primary mediastinal b-cell lymphoma, transformed follicular lymphoma, marginal zone lymphoma, mantle cell lymphoma, acute lymphoblastic leukemia including adult and pediatric ALL, non-Hodgkin lymphoma, indolent non-Hodgkin lymphoma, or chronic lymphocytic leukemia. For example, BCMA may be targeted in multiple myeloma or plasma cell leukemia (see, e.g., 2018 American Association for Cancer Research (AACR) Annual meeting Poster: Allogeneic Chimeric Antigen Receptor T Cells Targeting B Cell Maturation Antigen). For example, CLL1 may be targeted in acute myeloid leukemia. For example, MAGE A3, MAGE A6, SSX2, and/or KRAS may be targeted in solid tumors. For example, HPV E6 and/or HPV E7 may be targeted in cervical cancer or head and neck cancer. For example, WT1 may be targeted in acute myeloid leukemia (AML), myelodysplastic syndromes (MDS), chronic myeloid leukemia (CML), non-small cell lung cancer, breast, pancreatic, ovarian or colorectal cancers, or mesothelioma. For example, CD22 may be targeted in B cell malignancies, including non-Hodgkin lymphoma, diffuse large B-cell lymphoma, or acute lymphoblastic leukemia. For example, CD171 may be targeted in neuroblastoma, glioblastoma, or lung, pancreatic, or ovarian cancers. For example, ROR1 may be targeted in ROR1+ malignancies, including non-small cell lung cancer, triple negative breast cancer, pancreatic cancer, prostate cancer, ALL, chronic lymphocytic leukemia, or mantle cell lymphoma. For example, MUC16 may be targeted in MUC16ecto+ epithelial ovarian, fallopian tube or primary peritoneal cancer. For example, CD70 may be targeted in both hematologic malignancies as well as in solid cancers such as renal cell carcinoma (RCC), gliomas (e.g., GBM), and head and neck cancers (HNSCC). CD70 is expressed in both hematologic malignancies as well as in solid cancers, while its expression in normal tissues is restricted to a subset of lymphoid cell types (see, e.g., 2018 American Association for Cancer Research (AACR) Annual meeting Poster: Allogeneic CRISPR Engineered Anti-CD70 CAR-T Cells Demonstrate Potent Preclinical Activity Against Both Solid and Hematological Cancer Cells).

Various strategies may for example be employed to genetically modify T cells by altering the specificity of the T cell receptor (TCR) for example by introducing new TCR α and β chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379).

As an alternative to, or addition to, TCR modifications, chimeric antigen receptors (CARs) may be used in order to generate immunoresponsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and, PCT Publication WO9215322).

In general, CARs are comprised of an extracellular domain, a transmembrane domain, and an intracellular domain, wherein the extracellular domain comprises an antigen-binding domain that is specific for a predetermined target. While the antigen-binding domain of a CAR is often an antibody or antibody fragment (e.g., a single chain variable fragment, scFv), the binding domain is not particularly limited so long as it results in specific recognition of a target. For example, in some embodiments, the antigen-binding domain may comprise a receptor, such that the CAR is capable of binding to the ligand of the receptor. Alternatively, the antigen-binding domain may comprise a ligand, such that the CAR is capable of binding the endogenous receptor of that ligand.

The antigen-binding domain of a CAR is generally separated from the transmembrane domain by a hinge or spacer. The spacer is also not particularly limited, and it is designed to provide the CAR with flexibility. For example, a spacer domain may comprise a portion of a human Fc domain, including a portion of the CH3 domain, or the hinge region of any immunoglobulin, such as IgA, IgD, IgE, IgG, or IgM, or variants thereof. Furthermore, the hinge region may be modified so as to prevent off-target binding by FcRs or other potential interfering objects. For example, the hinge may comprise an IgG4 Fc domain with or without a S228P, L235E, and/or N297Q mutation (according to Kabat numbering) in order to decrease binding to FcRs. Additional spacers/hinges include, but are not limited to, CD4, CD8, and CD28 hinge regions.

The transmembrane domain of a CAR may be derived either from a natural or from a synthetic source. Where the source is natural, the domain may be derived from any membrane bound or transmembrane protein. Transmembrane regions of particular use in this disclosure may be derived from CD8, CD28, CD3, CD45, CD4, CD5, CDS, CD9, CD 16, CD22, CD33, CD37, CD64, CD80, CD86, CD 134, CD137, CD 154, TCR. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Alternative CAR constructs may be characterized as belonging to successive generations. First-generation CARs typically consist of a single-chain variable fragment of an antibody specific for an antigen, for example comprising a VL linked to a VH of a specific antibody, linked by a flexible linker, for example by a CD8α hinge domain and a CD8α transmembrane domain, to the transmembrane and intracellular signaling domains of either CD3ζ or FcRγ (scFv-CD3ζ or scFv-FcRγ; see U.S. Pat. Nos. 7,741,465; 5,912,172; 5,906,936). Second-generation CARs incorporate the intracellular domains of one or more costimulatory molecules, such as CD28, OX40 (CD134), or 4-1BB (CD137) within the endodomain (for example scFv-CD28/OX40/4-1BB-CD3ζ; see U.S. Pat. Nos. 8,911,993; 8,916,381; 8,975,071; 9,101,584; 9,102,760; 9,102,761). Third-generation CARs include a combination of costimulatory endodomains, such a CD3ζ-chain, CD97, GDI 1a-CD18, CD2, ICOS, CD27, CD154, CDS, OX40, 4-1BB, CD2, CD7, LIGHT, LFA-1, NKG2C, B7-H3, CD30, CD40, PD-1, or CD28 signaling domains (for example scFv-CD28-4-1BB-CD3ζ or scFv-CD28-OX40-CD3ζ; see U.S. Pat. Nos. 8,906,682; 8,399,645; 5,686,281; PCT Publication No. WO2014134165; PCT Publication No. WO2012079000). In certain embodiments, the primary signaling domain comprises a functional signaling domain of a protein selected from the group consisting of CD3 zeta, CD3 gamma, CD3 delta, CD3 epsilon, common FcR gamma (FCERIG), FcR beta (Fc Epsilon Rib), CD79a, CD79b, Fc gamma RIIa, DAP10, and DAP12. In certain preferred embodiments, the primary signaling domain comprises a functional signaling domain of CD3ζ or FcRγ. In certain embodiments, the one or more costimulatory signaling domains comprise a functional signaling domain of a protein selected, each independently, from the group consisting of: CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8 alpha, CD8 beta, IL2R beta, IL2R gamma, IL7R alpha, ITGA4, VLA1, CD49a, ITGA4, IA4, CD49D, ITGA6, VLA-6, CD49f, ITGAD, CD11 d, ITGAE, CD103, ITGAL, CD11a, LFA-1, ITGAM, CD 11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Lyl08), SLAM (SLAMFI, CD150, IPO-3), BLAME (SLAMF8), SELPLG (CD162), LTBR, LAT, GADS, SLP-76, PAG/Cbp, NKp44, NKp30, NKp46, and NKG2D. In certain embodiments, the one or more costimulatory signaling domains comprise a functional signaling domain of a protein selected, each independently, from the group consisting of: 4-1BB, CD27, and CD28. In certain embodiments, a chimeric antigen receptor may have the design as described in U.S. Pat. No. 7,446,190, comprising an intracellular domain of CD3ζ chain (such as amino acid residues 52-163 of the human CD3 zeta chain, as shown in SEQ ID NO: 14 of U.S. Pat. No. 7,446,190), a signaling region from CD28 and an antigen-binding element (or portion or domain; such as scFv). The CD28 portion, when between the zeta chain portion and the antigen-binding element, may suitably include the transmembrane and signaling domains of CD28 (such as amino acid residues 114-220 of SEQ ID NO: 10, full sequence shown in SEQ ID NO: 6 of U.S. Pat. No. 7,446,190; these can include the following portion of CD28 as set forth in Genbank identifier NM_006139 (sequence version 1, 2 or 3): IEVMYPPPYLDNEKSNGTIIHVKGKHLCPSPLFPGPSKPFWVLVVVGGVLACYSLLVTVA FIIFWVRSKRSRLLHSDYMNMTPRRPGPTRKHYQPYAPPRDFAAYRS) (SEQ ID NO:1)). Alternatively, when the zeta sequence lies between the CD28 sequence and the antigen-binding element, intracellular domain of CD28 can be used alone (such as amino sequence set forth in SEQ ID NO: 9 of U.S. Pat. No. 7,446,190). Hence, certain embodiments employ a CAR comprising (a) a zeta chain portion comprising the intracellular domain of human CD3ζ chain, (b) a costimulatory signaling region, and (c) an antigen-binding element (or portion or domain), wherein the costimulatory signaling region comprises the amino acid sequence encoded by SEQ ID NO: 6 of U.S. Pat. No. 7,446,190.

Alternatively, costimulation may be orchestrated by expressing CARs in antigen-specific T cells, chosen so as to be activated and expanded following engagement of their native αβTCR, for example by antigen on professional antigen-presenting cells, with attendant costimulation. In addition, additional engineered receptors may be provided on the immunoresponsive cells, for example to improve targeting of a T-cell attack and/or minimize side effects

By means of an example and without limitation, Kochenderfer et al., (2009) J Immunother. 32 (7): 689-702 described anti-CD19 chimeric antigen receptors (CAR). FMC63-28Z CAR contained a single chain variable region moiety (scFv) recognizing CD19 derived from the FMC63 mouse hybridoma (described in Nicholson et al., (1997) Molecular Immunology 34: 1157-1165), a portion of the human CD28 molecule, and the intracellular component of the human TCR-ζ molecule. FMC63-CD828BBZ CAR contained the FMC63 scFv, the hinge and transmembrane regions of the CD8 molecule, the cytoplasmic portions of CD28 and 4-1BB, and the cytoplasmic component of the TCR-ζ molecule. The exact sequence of the CD28 molecule included in the FMC63-28Z CAR corresponded to Genbank identifier NM_006139; the sequence included all amino acids starting with the amino acid sequence IEVMYPPPY (SEQ ID NO:2) and continuing all the way to the carboxy-terminus of the protein. To encode the anti-CD19 scFv component of the vector, the authors designed a DNA sequence which was based on a portion of a previously published CAR (Cooper et al., (2003) Blood 101: 1637-1644). This sequence encoded the following components in frame from the 5′ end to the 3′ end: an XhoI site, the human granulocyte-macrophage colony-stimulating factor (GM-CSF) receptor α-chain signal sequence, the FMC63 light chain variable region (as in Nicholson et al., supra), a linker peptide (as in Cooper et al., supra), the FMC63 heavy chain variable region (as in Nicholson et al., supra), and a NotI site. A plasmid encoding this sequence was digested with XhoI and NotI. To form the MSGV-FMC63-28Z retroviral vector, the XhoI and NotI-digested fragment encoding the FMC63 scFv was ligated into a second XhoI and NotI-digested fragment that encoded the MSGV retroviral backbone (as in Hughes et al., (2005) Human Gene Therapy 16: 457-472) as well as part of the extracellular portion of human CD28, the entire transmembrane and cytoplasmic portion of human CD28, and the cytoplasmic portion of the human TCR-ζ molecule (as in Maher et al., 2002) Nature Biotechnology 20: 70-75). The FMC63-28Z CAR is included in the KTE-C19 (axicabtagene ciloleucel) anti-CD19 CAR-T therapy product in development by Kite Pharma, Inc. for the treatment of inter alia patients with relapsed/refractory aggressive B-cell non-Hodgkin lymphoma (NHL). Accordingly, in certain embodiments, cells intended for adoptive cell therapies, more particularly immunoresponsive cells such as T cells, may express the FMC63-28Z CAR as described by Kochenderfer et al. (supra). Hence, in certain embodiments, cells intended for adoptive cell therapies, more particularly immunoresponsive cells such as T cells, may comprise a CAR comprising an extracellular antigen-binding element (or portion or domain; such as scFv) that specifically binds to an antigen, an intracellular signaling domain comprising an intracellular domain of a CD3ζ chain, and a costimulatory signaling region comprising a signaling domain of CD28. Preferably, the CD28 amino acid sequence is as set forth in Genbank identifier NM_006139 (sequence version 1, 2 or 3) starting with the amino acid sequence IEVMYPPPY and continuing all the way to the carboxy-terminus of the protein. The sequence is reproduced herein:

(SEQ ID NO: 3)
IEVMYPPPYLDNEKSNGTIIHVKGKHLCPSPLFPGPSKPFWVLVVVGGVL
ACYSLLVTVAFIIFWVRSKRSRLLHSDYMNMTPRRPGPTRKHYQPYAPPR
DFAAYRS.

Preferably, the antigen is CD19, more preferably the antigen-binding element is an anti-CD19 scFv, even more preferably the anti-CD19 scFv as described by Kochenderfer et al. (supra).

Additional anti-CD19 CARs are further described in WO2015187528. More particularly Example 1 and Table 1 of WO2015187528, incorporated by reference herein, demonstrate the generation of anti-CD19 CARs based on a fully human anti-CD19 monoclonal antibody (47G4, as described in US20100104509) and murine anti-CD19 monoclonal antibody (as described in Nicholson et al. and explained above). Various combinations of a signal sequence (human CD8-alpha or GM-CSF receptor), extracellular and transmembrane regions (human CD8-alpha) and intracellular T-cell signalling domains (CD28-CD3ζ; 4-1BB-CD3ζ; CD27-CD3ζ; CD28-CD27-CD3ζ, 4-1BB-CD27-CD3ζ; CD27-4-1BB-CD3ζ; CD28-CD27-FcεRI gamma chain; or CD28-FcεRI gamma chain) were disclosed. Hence, in certain embodiments, cells intended for adoptive cell therapies, more particularly immunoresponsive cells such as T cells, may comprise a CAR comprising an extracellular antigen-binding element that specifically binds to an antigen, an extracellular and transmembrane region as set forth in Table 1 of WO2015187528 and an intracellular T-cell signalling domain as set forth in Table 1 of WO2015187528. Preferably, the antigen is CD19, more preferably the antigen-binding element is an anti-CD19 scFv, even more preferably the mouse or human anti-CD19 scFv as described in Example 1 of WO2015187528. In certain embodiments, the CAR comprises, consists essentially of or consists of an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13 as set forth in Table 1 of WO2015187528.

By means of an example and without limitation, chimeric antigen receptor that recognizes the CD70 antigen is described in WO2012058460A2 (see also, Park et al., CD70 as a target for chimeric antigen receptor T cells in head and neck squamous cell carcinoma, Oral Oncol. 2018 March; 78:145-150; and Jin et al., CD70, a novel target of CAR T-cell therapy for gliomas, Neuro Oncol. 2018 Jan. 10; 20(1):55-65). CD70 is expressed by diffuse large B-cell and follicular lymphoma and also by the malignant cells of Hodgkins lymphoma, Waldenstrom's macroglobulinemia and multiple myeloma, and by HTLV-1- and EBV-associated malignancies. (Agathanggelou et al. Am. J. Pathol. 1995; 147: 1152-1160; Hunter et al., Blood 2004; 104:4881. 26; Lens et al., J Immunol. 2005; 174:6212-6219; Baba et al., J Virol. 2008; 82:3843-3852.) In addition, CD70 is expressed by non-hematological malignancies such as renal cell carcinoma and glioblastoma. (Junker et al., J Urol. 2005; 173:2150-2153; Chahlavi et al., Cancer Res 2005; 65:5428-5438) Physiologically, CD70 expression is transient and restricted to a subset of highly activated T, B, and dendritic cells.

By means of an example and without limitation, chimeric antigen receptor that recognizes BCMA has been described (see, e.g., US20160046724A1; WO2016014789A2; WO2017211900A1; WO2015158671A1; US20180085444A1; WO2018028647A1; US20170283504A1; and WO2013154760A1).

In certain embodiments, the immune cell may, in addition to a CAR or exogenous TCR as described herein, further comprise a chimeric inhibitory receptor (inhibitory CAR) that specifically binds to a second target antigen and is capable of inducing an inhibitory or immunosuppressive or repressive signal to the cell upon recognition of the second target antigen. In certain embodiments, the chimeric inhibitory receptor comprises an extracellular antigen-binding element (or portion or domain) configured to specifically bind to a target antigen, a transmembrane domain, and an intracellular immunosuppressive or repressive signaling domain. In certain embodiments, the second target antigen is an antigen that is not expressed on the surface of a cancer cell or infected cell or the expression of which is downregulated on a cancer cell or an infected cell. In certain embodiments, the second target antigen is an MHC-class I molecule. In certain embodiments, the intracellular signaling domain comprises a functional signaling portion of an immune checkpoint molecule, such as for example PD-1 or CTLA4. Advantageously, the inclusion of such inhibitory CAR reduces the chance of the engineered immune cells attacking non-target (e.g., non-cancer) tissues.

Alternatively, T-cells expressing CARs may be further modified to reduce or eliminate expression of endogenous TCRs in order to reduce off-target effects. Reduction or elimination of endogenous TCRs can reduce off-target effects and increase the effectiveness of the T cells (U.S. Pat. No. 9,181,527). T cells stably lacking expression of a functional TCR may be produced using a variety of approaches. T cells internalize, sort, and degrade the entire T cell receptor as a complex, with a half-life of about 10 hours in resting T cells and 3 hours in stimulated T cells (von Essen, M. et al. 2004. J. Immunol. 173:384-393). Proper functioning of the TCR complex requires the proper stoichiometric ratio of the proteins that compose the TCR complex. TCR function also requires two functioning TCR zeta proteins with ITAM motifs. The activation of the TCR upon engagement of its MHC-peptide ligand requires the engagement of several TCRs on the same T cell, which all must signal properly. Thus, if a TCR complex is destabilized with proteins that do not associate properly or cannot signal optimally, the T cell will not become activated sufficiently to begin a cellular response.

Accordingly, in some embodiments, TCR expression may eliminated using RNA interference (e.g., shRNA, siRNA, miRNA, etc.), CRISPR, or other methods that target the nucleic acids encoding specific TCRs (e.g., TCR-α and TCR-β) and/or CD3 chains in primary T cells. By blocking expression of one or more of these proteins, the T cell will no longer produce one or more of the key components of the TCR complex, thereby destabilizing the TCR complex and preventing cell surface expression of a functional TCR.

In some instances, CAR may also comprise a switch mechanism for controlling expression and/or activation of the CAR. For example, a CAR may comprise an extracellular, transmembrane, and intracellular domain, in which the extracellular domain comprises a target-specific binding element that comprises a label, binding domain, or tag that is specific for a molecule other than the target antigen that is expressed on or by a target cell. In such embodiments, the specificity of the CAR is provided by a second construct that comprises a target antigen binding domain (e.g., an scFv or a bispecific antibody that is specific for both the target antigen and the label or tag on the CAR) and a domain that is recognized by or binds to the label, binding domain, or tag on the CAR. See, e.g., WO 2013/044225, WO 2016/000304, WO 2015/057834, WO 2015/057852, WO 2016/070061, U.S. Pat. No. 9,233,125, US 2016/0129109. In this way, a T-cell that expresses the CAR can be administered to a subject, but the CAR cannot bind its target antigen until the second composition comprising an antigen-specific binding domain is administered.

Alternative switch mechanisms include CARs that require multimerization in order to activate their signaling function (see, e.g., US 2015/0368342, US 2016/0175359, US 2015/0368360) and/or an exogenous signal, such as a small molecule drug (US 2016/0166613, Yung et al., Science, 2015), in order to elicit a T-cell response. Some CARs may also comprise a “suicide switch” to induce cell death of the CAR T-cells following treatment (Buddee et al., PLoS One, 2013) or to downregulate expression of the CAR following binding to the target antigen (WO 2016/011210).

Alternative techniques may be used to transform target immunoresponsive cells, such as protoplast fusion, lipofection, transfection or electroporation. A wide variety of vectors may be used, such as retroviral vectors, lentiviral vectors, adenoviral vectors, adeno-associated viral vectors, plasmids or transposons, such as a Sleeping Beauty transposon (see U.S. Pat. Nos. 6,489,458; 7,148,203; 7,160,682; 7,985,739; 8,227,432), may be used to introduce CARs, for example using 2nd generation antigen-specific CARs signaling through CD3ζ and either CD28 or CD137. Viral vectors may for example include vectors based on HIV, SV40, EBV, HSV or BPV.

Cells that are targeted for transformation may for example include T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells may be differentiated. T cells expressing a desired CAR may for example be selected through co-culture with γ-irradiated activating and propagating cells (AaPC), which co-express the cancer antigen and co-stimulatory molecules. The engineered CAR T-cells may be expanded, for example by co-culture on AaPC in presence of soluble factors, such as IL-2 and IL-21. This expansion may for example be carried out so as to provide memory CAR+ T cells (which may for example be assayed by non-enzymatic digital array and/or multi-panel flow cytometry). In this way, CAR T cells may be provided that have specific cytotoxic activity against antigen-bearing tumors (optionally in conjunction with production of desired chemokines such as interferon-γ). CAR T cells of this kind may for example be used in animal models, for example to treat tumor xenografts.

In certain embodiments, ACT includes co-transferring CD4+ Th1 cells and CD8+ CTLs to induce a synergistic antitumour response (see, e.g., Li et al., Adoptive cell therapy with CD4+ T helper 1 cells and CD8+ cytotoxic T cells enhances complete rejection of an established tumour, leading to generation of endogenous memory responses to non-targeted tumour epitopes. Clin Transl Immunology. 2017 October; 6(10): e160).

In certain embodiments, Th17 cells are transferred to a subject in need thereof. Th17 cells have been reported to directly eradicate melanoma tumors in mice to a greater extent than Th1 cells (Muranski P, et al., Tumor-specific Th17-polarized cells eradicate large established melanoma. Blood. 2008 Jul. 15; 112(2):362-73; and Martin-Orozco N, et al., T helper 17 cells promote cytotoxic T cell activation in tumor immunity. Immunity. 2009 Nov. 20; 31(5):787-98). Those studies involved an adoptive T cell transfer (ACT) therapy approach, which takes advantage of CD4+ T cells that express a TCR recognizing tyrosinase tumor antigen. Exploitation of the TCR leads to rapid expansion of Th17 populations to large numbers ex vivo for reinfusion into the autologous tumor-bearing hosts.

In certain embodiments, ACT may include autologous iPSC-based vaccines, such as irradiated iPSCs in autologous anti-tumor vaccines (see e.g., Kooreman, Nigel G. et al., Autologous iPSC-Based Vaccines Elicit Anti-tumor Responses In Vivo, Cell Stem Cell 22, 1-13, 2018, doi.org/10.1016/j.stem.2018.01.016).

Unlike T-cell receptors (TCRs) that are MHC restricted, CARs can potentially bind any cell surface-expressed antigen and can thus be more universally used to treat patients (see Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267). In certain embodiments, in the absence of endogenous T-cell infiltrate (e.g., due to aberrant antigen processing and presentation), which precludes the use of TIL therapy and immune checkpoint blockade, the transfer of CAR T-cells may be used to treat patients (see, e.g., Hinrichs C S, Rosenberg S A. Exploiting the curative potential of adoptive T-cell therapy for cancer. Immunol Rev (2014) 257(1):56-71. doi:10.1111/imr.12132).

Approaches such as the foregoing may be adapted to provide methods of treating and/or increasing survival of a subject having a disease, such as a neoplasia, for example by administering an effective amount of an immunoresponsive cell comprising an antigen recognizing receptor that binds a selected antigen, wherein the binding activates the immunoresponsive cell, thereby treating or preventing the disease (such as a neoplasia, a pathogen infection, an autoimmune disorder, or an allogeneic transplant reaction).

In certain embodiments, the treatment can be administered after lymphodepleting pretreatment in the form of chemotherapy (typically a combination of cyclophosphamide and fludarabine) or radiation therapy. Initial studies in ACT had short lived responses and the transferred cells did not persist in vivo for very long (Houot et al., T-cell-based immunotherapy: adoptive cell transfer and checkpoint inhibition. Cancer Immunol Res (2015) 3(10):1115-22; and Kamta et al., Advancing Cancer Therapy with Present and Emerging Immuno-Oncology Approaches. Front. Oncol. (2017) 7:64). Immune suppressor cells like Tregs and MDSCs may attenuate the activity of transferred cells by outcompeting them for the necessary cytokines. Not being bound by a theory lymphodepleting pretreatment may eliminate the suppressor cells allowing the TILs to persist.

In one embodiment, the treatment can be administrated into patients undergoing an immunosuppressive treatment (e.g., glucocorticoid treatment). The cells or population of cells, may be made resistant to at least one immunosuppressive agent due to the inactivation of a gene encoding a receptor for such immunosuppressive agent. In certain embodiments, the immunosuppressive treatment provides for the selection and expansion of the immunoresponsive T cells within the patient.

In certain embodiments, the treatment can be administered before primary treatment (e.g., surgery or radiation therapy) to shrink a tumor before the primary treatment. In another embodiment, the treatment can be administered after primary treatment to remove any remaining cancer cells.

In certain embodiments, immunometabolic barriers can be targeted therapeutically prior to and/or during ACT to enhance responses to ACT or CAR T-cell therapy and to support endogenous immunity (see, e.g., Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267).

The administration of cells or population of cells, such as immune system cells or cell populations, such as more particularly immunoresponsive cells or cell populations, as disclosed herein may be carried out in any convenient manner, including by aerosol inhalation, injection, ingestion, transfusion, implantation or transplantation. The cells or population of cells may be administered to a patient subcutaneously, intradermally, intratumorally, intranodally, intramedullary, intramuscularly, intrathecally, by intravenous or intralymphatic injection, or intraperitoneally. In some embodiments, the disclosed CARs may be delivered or administered into a cavity formed by the resection of tumor tissue (i.e. intracavity delivery) or directly into a tumor prior to resection (i.e. intratumoral delivery). In one embodiment, the cell compositions of the present invention are preferably administered by intravenous injection.

The administration of the cells or population of cells can consist of the administration of 10⁴-10⁹ cells per kg body weight, preferably 10⁵ to 10⁶ cells/kg body weight including all integer values of cell numbers within those ranges. Dosing in CAR T cell therapies may for example involve administration of from 10⁶ to 10⁹ cells/kg, with or without a course of lymphodepletion, for example with cyclophosphamide. The cells or population of cells can be administrated in one or more doses. In another embodiment, the effective amount of cells are administrated as a single dose. In another embodiment, the effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, the effective amount of cells or composition comprising those cells are administrated parenterally. The administration can be an intravenous administration. The administration can be directly done by injection within a tumor.

To guard against possible adverse reactions, engineered immunoresponsive cells may be equipped with a transgenic safety switch, in the form of a transgene that renders the cells vulnerable to exposure to a specific signal. For example, the herpes simplex viral thymidine kinase (TK) gene may be used in this way, for example by introduction into allogeneic T lymphocytes used as donor lymphocyte infusions following stem cell transplantation (Greco, et al., Improving the safety of cell therapy with the TK-suicide gene. Front. Pharmacol. 2015; 6: 95). In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 20130071414; PCT Patent Publication WO2011146862; PCT Patent Publication WO2014011987; PCT Patent Publication WO2013040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28(6):1107-15 (2010)).

In a further refinement of adoptive therapies, genome editing may be used to tailor immunoresponsive cells to alternative implementations, for example providing edited CAR T cells (see Poirot et al., 2015, Multiplex genome edited T-cell manufacturing platform for “off-the-shelf” adoptive T-cell immunotherapies, Cancer Res 75 (18): 3853; Ren et al., 2017, Multiplex genome editing to generate universal CAR T cells resistant to PD1 inhibition, Clin Cancer Res. 2017 May 1; 23(9):2255-2266. doi: 10.1158/1078-0432.CCR-16-1300. Epub 2016 Nov. 4; Qasim et al., 2017, Molecular remission of infant B-ALL after infusion of universal TALEN gene-edited CAR T cells, Sci Transl Med. 2017 Jan. 25; 9(374); Legut, et al., 2018, CRISPR-mediated TCR replacement generates superior anticancer transgenic T cells. Blood, 131(3), 311-322; and Georgiadis et al., Long Terminal Repeat CRISPR-CAR-Coupled “Universal” T Cells Mediate Potent Anti-leukemic Effects, Molecular Therapy, In Press, Corrected Proof, Available online 6 Mar. 2018). Cells may be edited using any CRISPR system and method of use thereof as described herein. CRISPR systems may be delivered to an immune cell by any method described herein. In preferred embodiments, cells are edited ex vivo and transferred to a subject in need thereof. Immunoresponsive cells, CAR T cells or any cells used for adoptive cell transfer may be edited. Editing may be performed for example to insert or knock-in an exogenous gene, such as an exogenous gene encoding a CAR or a TCR, at a preselected locus in a cell (e.g. TRAC locus); to eliminate potential alloreactive T-cell receptors (TCR) or to prevent inappropriate pairing between endogenous and exogenous TCR chains, such as to knock-out or knock-down expression of an endogenous TCR in a cell; to disrupt the target of a chemotherapeutic agent in a cell; to block an immune checkpoint, such as to knock-out or knock-down expression of an immune checkpoint protein or receptor in a cell; to knock-out or knock-down expression of other gene or genes in a cell, the reduced expression or lack of expression of which can enhance the efficacy of adoptive therapies using the cell; to knock-out or knock-down expression of an endogenous gene in a cell, said endogenous gene encoding an antigen targeted by an exogenous CAR or TCR; to knock-out or knock-down expression of one or more MHC constituent proteins in a cell; to activate a T cell; to modulate cells such that the cells are resistant to exhaustion or dysfunction; and/or increase the differentiation and/or proliferation of functionally exhausted or dysfunctional CD8+ T-cells (see PCT Patent Publications: WO2013176915, WO2014059173, WO2014172606, WO2014184744, and WO2014191128).

In certain embodiments, editing may result in inactivation of a gene. By inactivating a gene, it is intended that the gene of interest is not expressed in a functional protein form. In a particular embodiment, the CRISPR system specifically catalyzes cleavage in one targeted gene thereby inactivating said targeted gene. The nucleic acid strand breaks caused are commonly repaired through the distinct mechanisms of homologous recombination or non-homologous end joining (NHEJ). However, NHEJ is an imperfect repair process that often results in changes to the DNA sequence at the site of the cleavage. Repair via non-homologous end joining (NHEJ) often results in small insertions or deletions (Indel) and can be used for the creation of specific gene knockouts. Cells in which a cleavage induced mutagenesis event has occurred can be identified and/or selected by well-known methods in the art. In certain embodiments, homology directed repair (HDR) is used to concurrently inactivate a gene (e.g., TRAC) and insert an endogenous TCR or CAR into the inactivated locus.

Hence, in certain embodiments, editing of cells (such as by CRISPR/Cas), particularly cells intended for adoptive cell therapies, more particularly immunoresponsive cells such as T cells, may be performed to insert or knock-in an exogenous gene, such as an exogenous gene encoding a CAR or a TCR, at a preselected locus in a cell. Conventionally, nucleic acid molecules encoding CARs or TCRs are transfected or transduced to cells using randomly integrating vectors, which, depending on the site of integration, may lead to clonal expansion, oncogenic transformation, variegated transgene expression and/or transcriptional silencing of the transgene. Directing of transgene(s) to a specific locus in a cell can minimize or avoid such risks and advantageously provide for uniform expression of the transgene(s) by the cells. Without limitation, suitable ‘safe harbor’ loci for directed transgene integration include CCR5 or AAVS1. Homology-directed repair (HDR) strategies are known and described elsewhere in this specification allowing to insert transgenes into desired loci (e.g., TRAC locus).

Further suitable loci for insertion of transgenes, in particular CAR or exogenous TCR transgenes, include without limitation loci comprising genes coding for constituents of endogenous T-cell receptor, such as T-cell receptor alpha locus (TRA) or T-cell receptor beta locus (TRB), for example T-cell receptor alpha constant (TRAC) locus, T-cell receptor beta constant 1 (TRBC1) locus or T-cell receptor beta constant 2 (TRBC1) locus. Advantageously, insertion of a transgene into such locus can simultaneously achieve expression of the transgene, potentially controlled by the endogenous promoter, and knock-out expression of the endogenous TCR. This approach has been exemplified in Eyquem et al., (2017) Nature 543: 113-117, wherein the authors used CRISPR/Cas9 gene editing to knock-in a DNA molecule encoding a CD19-specific CAR into the TRAC locus downstream of the endogenous promoter; the CAR-T cells obtained by CRISPR were significantly superior in terms of reduced tonic CAR signaling and exhaustion.

T cell receptors (TCR) are cell surface receptors that participate in the activation of T cells in response to the presentation of antigen. The TCR is generally made from two chains, α and β, which assemble to form a heterodimer and associates with the CD3-transducing subunits to form the T cell receptor complex present on the cell surface. Each α and β chain of the TCR consists of an immunoglobulin-like N-terminal variable (V) and constant (C) region, a hydrophobic transmembrane domain, and a short cytoplasmic region. As for immunoglobulin molecules, the variable region of the α and β chains are generated by V(D)J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of graft versus host disease (GVHD). The inactivation of TCRa or TCRP can result in the elimination of the TCR from the surface of T cells preventing recognition of alloantigen and thus GVHD. However, TCR disruption generally results in the elimination of the CD3 signaling component and alters the means of further T cell expansion.

Hence, in certain embodiments, editing of cells (such as by CRISPR/Cas), particularly cells intended for adoptive cell therapies, more particularly immunoresponsive cells such as T cells, may be performed to knock-out or knock-down expression of an endogenous TCR in a cell. For example, NHEJ-based or HDR-based gene editing approaches can be employed to disrupt the endogenous TCR alpha and/or beta chain genes. For example, gene editing system or systems, such as CRISPR/Cas system or systems, can be designed to target a sequence found within the TCR beta chain conserved between the beta 1 and beta 2 constant region genes (TRBC1 and TRBC2) and/or to target the constant region of the TCR alpha chain (TRAC) gene.

Allogeneic cells are rapidly rejected by the host immune system. It has been demonstrated that, allogeneic leukocytes present in non-irradiated blood products will persist for no more than 5 to 6 days (Boni, Muranski et al. 2008 Blood 1; 112(12):4746-54). Thus, to prevent rejection of allogeneic cells, the host's immune system usually has to be suppressed to some extent. However, in the case of adoptive cell transfer the use of immunosuppressive drugs also have a detrimental effect on the introduced therapeutic T cells. Therefore, to effectively use an adoptive immunotherapy approach in these conditions, the introduced cells would need to be resistant to the immunosuppressive treatment. Thus, in a particular embodiment, the present invention further comprises a step of modifying T cells to make them resistant to an immunosuppressive agent, preferably by inactivating at least one gene encoding a target for an immunosuppressive agent. An immunosuppressive agent is an agent that suppresses immune function by one of several mechanisms of action. An immunosuppressive agent can be, but is not limited to a calcineurin inhibitor, a target of rapamycin, an interleukin-2 receptor α-chain blocker, an inhibitor of inosine monophosphate dehydrogenase, an inhibitor of dihydrofolic acid reductase, a corticosteroid or an immunosuppressive antimetabolite. The present invention allows conferring immunosuppressive resistance to T cells for immunotherapy by inactivating the target of the immunosuppressive agent in T cells. As non-limiting examples, targets for an immunosuppressive agent can be a receptor for an immunosuppressive agent such as: CD52, glucocorticoid receptor (GR), a FKBP family gene member and a cyclophilin family gene member.

In certain embodiments, editing of cells (such as by CRISPR/Cas), particularly cells intended for adoptive cell therapies, more particularly immunoresponsive cells such as T cells, may be performed to block an immune checkpoint, such as to knock-out or knock-down expression of an immune checkpoint protein or receptor in a cell. Immune checkpoints are inhibitory pathways that slow down or stop immune reactions and prevent excessive tissue damage from uncontrolled activity of immune cells. In certain embodiments, the immune checkpoint targeted is the programmed death-1 (PD-1 or CD279) gene (PDCD1). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL1 or KIR. In further additional embodiments, the immune checkpoint targeted is a member of the TNFR superfamily such as CD40, OX40, CD137, GITR, CD27 or TIM-3.

Additional immune checkpoints include Src homology 2 domain-containing protein tyrosine phosphatase 1 (SHP-1) (Watson H A, et al., SHP-1: the next checkpoint target for cancer immunotherapy? Biochem Soc Trans. 2016 Apr. 15; 44(2):356-62). SHP-1 is a widely expressed inhibitory protein tyrosine phosphatase (PTP). In T-cells, it is a negative regulator of antigen-dependent activation and proliferation. It is a cytosolic protein, and therefore not amenable to antibody-mediated therapies, but its role in activation and proliferation makes it an attractive target for genetic manipulation in adoptive transfer strategies, such as chimeric antigen receptor (CAR) T cells. Immune checkpoints may also include T cell immunoreceptor with Ig and ITIM domains (TIGIT/Vstm3/WUCAM/VSIG9) and VISTA (Le Mercier I, et al., (2015) Beyond CTLA-4 and PD-1, the generation Z of negative checkpoint regulators. Front. Immunol. 6:418).

WO2014172606 relates to the use of MT1 and/or MT2 inhibitors to increase proliferation and/or activity of exhausted CD8+ T-cells and to decrease CD8+ T-cell exhaustion (e.g., decrease functionally exhausted or unresponsive CD8+ immune cells). In certain embodiments, metallothioneins are targeted by gene editing in adoptively transferred T cells.

In certain embodiments, targets of gene editing may be at least one targeted locus involved in the expression of an immune checkpoint protein. Such targets may include, but are not limited to CTLA4, PPP2CA, PPP2CB, PTPN6, PTPN22, PDCD1, ICOS (CD278), PDL1, KIR, LAG3, HAVCR2, BTLA, CD160, TIGIT, CD96, CRTAM, LAIR1, SIGLEC7, SIGLEC9, CD244 (2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, VISTA, GUCYlA2, GUCYlA3, GUCYlB2, GUCYlB3, MT1, MT2, CD40, OX40, CD137, GITR, CD27, SHP-1, TIM-3, CEACAM-1, CEACAM-3, or CEACAM-5. In preferred embodiments, the gene locus involved in the expression of PD-1 or CTLA-4 genes is targeted. In other preferred embodiments, combinations of genes are targeted, such as but not limited to PD-1 and TIGIT.

By means of an example and without limitation, WO2016196388 concerns an engineered T cell comprising (a) a genetically engineered antigen receptor that specifically binds to an antigen, which receptor may be a CAR; and (b) a disrupted gene encoding a PD-L1, an agent for disruption of a gene encoding a PD-L1, and/or disruption of a gene encoding PD-L1, wherein the disruption of the gene may be mediated by a gene editing nuclease, a zinc finger nuclease (ZFN), CRISPR/Cas9 and/or TALEN. WO2015142675 relates to immune effector cells comprising a CAR in combination with an agent (such as CRISPR, TALEN or ZFN) that increases the efficacy of the immune effector cells in the treatment of cancer, wherein the agent may inhibit an immune inhibitory molecule, such as PD1, PD-L1, CTLA-4, TIM-3, LAG-3, VISTA, BTLA, TIGIT, LAIR1, CD160, 2B4, TGFR beta, CEACAM-1, CEACAM-3, or CEACAM-5. Ren et al., (2017) Clin Cancer Res 23 (9) 2255-2266 performed lentiviral delivery of CAR and electro-transfer of Cas9 mRNA and gRNAs targeting endogenous TCR, β-2 microglobulin (B2M) and PD1 simultaneously, to generate gene-disrupted allogeneic CAR T cells deficient of TCR, HLA class I molecule and PD1.

In certain embodiments, cells may be engineered to express a CAR, wherein expression and/or function of methylcytosine dioxygenase genes (TET1, TET2 and/or TET3) in the cells has been reduced or eliminated, such as by CRISPR, ZNF or TALEN (for example, as described in WO201704916).

In certain embodiments, editing of cells (such as by CRISPR/Cas), particularly cells intended for adoptive cell therapies, more particularly immunoresponsive cells such as T cells, may be performed to knock-out or knock-down expression of an endogenous gene in a cell, said endogenous gene encoding an antigen targeted by an exogenous CAR or TCR, thereby reducing the likelihood of targeting of the engineered cells. In certain embodiments, the targeted antigen may be one or more antigen selected from the group consisting of CD38, CD138, CS-1, CD33, CD26, CD30, CD53, CD92, CD100, CD148, CD150, CD200, CD261, CD262, CD362, human telomerase reverse transcriptase (hTERT), survivin, mouse double minute 2 homolog (MDM2), cytochrome P450 1B1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (D1), B cell maturation antigen (BCMA), transmembrane activator and CAML Interactor (TACI), and B-cell activating factor receptor (BAFF-R) (for example, as described in WO2016011210 and WO2017011804).

In certain embodiments, editing of cells (such as by CRISPR/Cas), particularly cells intended for adoptive cell therapies, more particularly immunoresponsive cells such as T cells, may be performed to knock-out or knock-down expression of one or more MHC constituent proteins, such as one or more HLA proteins and/or beta-2 microglobulin (B2M), in a cell, whereby rejection of non-autologous (e.g., allogeneic) cells by the recipient's immune system can be reduced or avoided. In preferred embodiments, one or more HLA class I proteins, such as HLA-A, B and/or C, and/or B2M may be knocked-out or knocked-down. Preferably, B2M may be knocked-out or knocked-down. By means of an example, Ren et al., (2017) Clin Cancer Res 23 (9) 2255-2266 performed lentiviral delivery of CAR and electro-transfer of Cas9 mRNA and gRNAs targeting endogenous TCR, 3-2 microglobulin (B2M) and PD1 simultaneously, to generate gene-disrupted allogeneic CAR T cells deficient of TCR, HLA class I molecule and PD1.

In other embodiments, at least two genes are edited. Pairs of genes may include, but are not limited to PD1 and TCRα, PD1 and TCRβ, CTLA-4 and TCRα, CTLA-4 and TCRβ, LAG3 and TCRα, LAG3 and TCRβ, Tim3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ, B2M and TCRα, B2M and TCRβ.

In certain embodiments, a cell may be multiply edited (multiplex genome editing) as taught herein to (1) knock-out or knock-down expression of an endogenous TCR (for example, TRBC1, TRBC2 and/or TRAC), (2) knock-out or knock-down expression of an immune checkpoint protein or receptor (for example PD1, PD-L1 and/or CTLA4); and (3) knock-out or knock-down expression of one or more MHC constituent proteins (for example, HLA-A, B and/or C, and/or B2M, preferably B2M).

Whether prior to or after genetic modification of the T cells, the T cells can be activated and expanded generally using methods as described, for example, in U.S. Pat. Nos. 6,352,694; 6,534,055; 6,905,680; 5,858,358; 6,887,466; 6,905,681; 7,144,575; 7,232,566; 7,175,843; 5,883,223; 6,905,874; 6,797,514; 6,867,041; and 7,572,631. T cells can be expanded in vitro or in vivo.

Immune cells may be obtained using any method known in the art. In one embodiment, allogenic T cells may be obtained from healthy subjects. In one embodiment T cells that have infiltrated a tumor are isolated. T cells may be removed during surgery. T cells may be isolated after removal of tumor tissue by biopsy. T cells may be isolated by any means known in the art. In one embodiment, T cells are obtained by apheresis. In one embodiment, the method may comprise obtaining a bulk population of T cells from a tumor sample by any suitable method known in the art. For example, a bulk population of T cells can be obtained from a tumor sample by dissociating the tumor sample into a cell suspension from which specific cell populations can be selected. Suitable methods of obtaining a bulk population of T cells may include, but are not limited to, any one or more of mechanically dissociating (e.g., mincing) the tumor, enzymatically dissociating (e.g., digesting) the tumor, and aspiration (e.g., as with a needle).

The bulk population of T cells obtained from a tumor sample may comprise any suitable type of T cell. Preferably, the bulk population of T cells obtained from a tumor sample comprises tumor infiltrating lymphocytes (TILs).

The tumor sample may be obtained from any mammal. Unless stated otherwise, as used herein, the term “mammal” refers to any mammal including, but not limited to, mammals of the order Logomorpha, such as rabbits; the order Carnivora, including Felines (cats) and Canines (dogs); the order Artiodactyla, including Bovines (cows) and Swines (pigs); or of the order Perssodactyla, including Equines (horses). The mammals may be non-human primates, e.g., of the order Primates, Ceboids, or Simoids (monkeys) or of the order Anthropoids (humans and apes). In some embodiments, the mammal may be a mammal of the order Rodentia, such as mice and hamsters. Preferably, the mammal is a non-human primate or a human. An especially preferred mammal is the human.

T cells can be obtained from a number of sources, including peripheral blood mononuclear cells, bone marrow, lymph node tissue, spleen tissue, and tumors. In certain embodiments of the present invention, T cells can be obtained from a unit of blood collected from a subject using any number of techniques known to the skilled artisan, such as Ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor) according to the manufacturer's instructions. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient. A specific subpopulation of T cells, such as CD28+, CD4+, CDC, CD45RA+, and CD45RO+ T cells, can be further isolated by positive or negative selection techniques. For example, in one preferred embodiment, T cells are isolated by incubation with anti-CD3/anti-CD28 (i.e., 3×28)-conjugated beads, such as DYNABEADS® M-450 CD3/CD28 T, or XCYTE DYNABEADS™ for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells.

Enrichment of a T cell population by negative selection can be accomplished with a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

Further, monocyte populations (i.e., CD14+ cells) may be depleted from blood preparations by a variety of methodologies, including anti-CD14 coated beads or columns, or utilization of the phagocytotic activity of these cells to facilitate removal. Accordingly, in one embodiment, the invention uses paramagnetic particles of a size sufficient to be engulfed by phagocytotic monocytes. In certain embodiments, the paramagnetic particles are commercially available beads, for example, those produced by Life Technologies under the trade name Dynabeads™. In one embodiment, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be isolated. In certain embodiments, the irrelevant beads include beads coated with sheep anti-mouse antibodies, goat anti-mouse antibodies, and human serum albumin.

In brief, such depletion of monocytes is performed by preincubating T cells isolated from whole blood, apheresed peripheral blood, or tumors with one or more varieties of irrelevant or non-antibody coupled paramagnetic particles at any amount that allows for removal of monocytes (approximately a 20:1 bead:cell ratio) for about 30 minutes to 2 hours at 22 to 37 degrees C., followed by magnetic removal of cells which have attached to or engulfed the paramagnetic particles. Such separation can be performed using standard methods available in the art. For example, any magnetic separation methodology may be used including a variety of which are commercially available, (e.g., DYNAL® Magnetic Particle Concentrator (DYNAL MPC®)). Assurance of requisite depletion can be monitored by a variety of methodologies known to those of ordinary skill in the art, including flow cytometric analysis of CD14 positive cells, before and after depletion.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc). Such populations of cells may have therapeutic value and would be desirable to obtain. For example, using high concentration of cells allows more efficient selection of CD8+ T cells that normally have weaker CD28 expression.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one embodiment, the concentration of cells used is 5×10⁶/ml. In other embodiments, the concentration used can be from about 1×10⁵/ml to 1×10⁶/ml, and any integer value in between.

T cells can also be frozen. Wishing not to be bound by theory, the freeze and subsequent thaw step provides a more uniform product by removing granulocytes and to some extent monocytes in the cell population. After a washing step to remove plasma and platelets, the cells may be suspended in a freezing solution. While many freezing solutions and parameters are known in the art and will be useful in this context, one method involves using PBS containing 20% DMSO and 8% human serum albumin, or other suitable cell freezing media, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

T cells for use in the present invention may also be antigen-specific T cells. For example, tumor-specific T cells can be used. In certain embodiments, antigen-specific T cells can be isolated from a patient of interest, such as a patient afflicted with a cancer or an infectious disease. In one embodiment, neoepitopes are determined for a subject and T cells specific to these antigens are isolated. Antigen-specific cells for use in expansion may also be generated in vitro using any number of methods known in the art, for example, as described in U.S. Patent Publication No. US 20040224402 entitled, Generation and Isolation of Antigen-Specific T Cells, or in U.S. Pat. No. 6,040,177. Antigen-specific cells for use in the present invention may also be generated using any number of methods known in the art, for example, as described in Current Protocols in Immunology, or Current Protocols in Cell Biology, both published by John Wiley & Sons, Inc., Boston, Mass.

In a related embodiment, it may be desirable to sort or otherwise positively select (e.g. via magnetic selection) the antigen specific cells prior to or following one or two rounds of expansion. Sorting or positively selecting antigen-specific cells can be carried out using peptide-MIIC tetramers (Altman, et al., Science. 1996 Oct. 4; 274(5284):94-6). In another embodiment, the adaptable tetramer technology approach is used (Andersen et al., 2012 Nat Protoc. 7:891-902). Tetramers are limited by the need to utilize predicted binding peptides based on prior hypotheses, and the restriction to specific HLAs. Peptide-MHIC tetramers can be generated using techniques known in the art and can be made with any MIIC molecule of interest and any antigen of interest as described herein. Specific epitopes to be used in this context can be identified using numerous assays known in the art. For example, the ability of a polypeptide to bind to MIIC class I may be evaluated indirectly by monitoring the ability to promote incorporation of ¹²⁵I labeled β2-microglobulin (β2m) into MIIC class I/β2m/peptide heterotrimeric complexes (see Parker et al., J. Immunol. 152:163, 1994).

In one embodiment cells are directly labeled with an epitope-specific reagent for isolation by flow cytometry followed by characterization of phenotype and TCRs. In one embodiment, T cells are isolated by contacting with T cell specific antibodies. Sorting of antigen-specific T cells, or generally any cells of the present invention, can be carried out using any of a variety of commercially available cell sorters, including, but not limited to, MoFlo sorter (DakoCytomation, Fort Collins, Colo.), FACSAria™, FACSArray™, FACSVantage™, BD™ LSR II, and FACSCalibur™ (BD Biosciences, San Jose, Calif.).

In a preferred embodiment, the method comprises selecting cells that also express CD3. The method may comprise specifically selecting the cells in any suitable manner. Preferably, the selecting is carried out using flow cytometry. The flow cytometry may be carried out using any suitable method known in the art. The flow cytometry may employ any suitable antibodies and stains. Preferably, the antibody is chosen such that it specifically recognizes and binds to the particular biomarker being selected. For example, the specific selection of CD3, CD8, TIM-3, LAG-3, 4-1BB, or PD-1 may be carried out using anti-CD3, anti-CD8, anti-TIM-3, anti-LAG-3, anti-4-1BB, or anti-PD-1 antibodies, respectively. The antibody or antibodies may be conjugated to a bead (e.g., a magnetic bead) or to a fluorochrome. Preferably, the flow cytometry is fluorescence-activated cell sorting (FACS). TCRs expressed on T cells can be selected based on reactivity to autologous tumors. Additionally, T cells that are reactive to tumors can be selected for based on markers using the methods described in patent publication Nos. WO2014133567 and WO2014133568, herein incorporated by reference in their entirety. Additionally, activated T cells can be selected for based on surface expression of CD107a.

In one embodiment of the invention, the method further comprises expanding the numbers of T cells in the enriched cell population. Such methods are described in U.S. Pat. No. 8,637,307 and is herein incorporated by reference in its entirety. The numbers of T cells may be increased at least about 3-fold (or 4-, 5-, 6-, 7-, 8-, or 9-fold), more preferably at least about 10-fold (or 20-, 30-, 40-, 50-, 60-, 70-, 80-, or 90-fold), more preferably at least about 100-fold, more preferably at least about 1,000 fold, or most preferably at least about 100,000-fold. The numbers of T cells may be expanded using any suitable method known in the art. Exemplary methods of expanding the numbers of cells are described in patent publication No. WO 2003057171, U.S. Pat. No. 8,034,334, and U.S. Patent Application Publication No. 2012/0244133, each of which is incorporated herein by reference.

In one embodiment, ex vivo T cell expansion can be performed by isolation of T cells and subsequent stimulation or activation followed by further expansion. In one embodiment of the invention, the T cells may be stimulated or activated by a single agent. In another embodiment, T cells are stimulated or activated with two agents, one that induces a primary signal and a second that is a co-stimulatory signal. Ligands useful for stimulating a single signal or stimulating a primary signal and an accessory molecule that stimulates a second signal may be used in soluble form. Ligands may be attached to the surface of a cell, to an Engineered Multivalent Signaling Platform (EMSP), or immobilized on a surface. In a preferred embodiment both primary and secondary agents are co-immobilized on a surface, for example a bead or a cell. In one embodiment, the molecule providing the primary activation signal may be a CD3 ligand, and the co-stimulatory molecule may be a CD28 ligand or 4-1BB ligand.

In certain embodiments, T cells comprising a CAR or an exogenous TCR, may be manufactured as described in WO2015120096, by a method comprising: enriching a population of lymphocytes obtained from a donor subject; stimulating the population of lymphocytes with one or more T-cell stimulating agents to produce a population of activated T cells, wherein the stimulation is performed in a closed system using serum-free culture medium; transducing the population of activated T cells with a viral vector comprising a nucleic acid molecule which encodes the CAR or TCR, using a single cycle transduction to produce a population of transduced T cells, wherein the transduction is performed in a closed system using serum-free culture medium; and expanding the population of transduced T cells for a predetermined time to produce a population of engineered T cells, wherein the expansion is performed in a closed system using serum-free culture medium. In certain embodiments, T cells comprising a CAR or an exogenous TCR, may be manufactured as described in WO2015120096, by a method comprising: obtaining a population of lymphocytes; stimulating the population of lymphocytes with one or more stimulating agents to produce a population of activated T cells, wherein the stimulation is performed in a closed system using serum-free culture medium; transducing the population of activated T cells with a viral vector comprising a nucleic acid molecule which encodes the CAR or TCR, using at least one cycle transduction to produce a population of transduced T cells, wherein the transduction is performed in a closed system using serum-free culture medium; and expanding the population of transduced T cells to produce a population of engineered T cells, wherein the expansion is performed in a closed system using serum-free culture medium. The predetermined time for expanding the population of transduced T cells may be 3 days. The time from enriching the population of lymphocytes to producing the engineered T cells may be 6 days. The closed system may be a closed bag system. Further provided is population of T cells comprising a CAR or an exogenous TCR obtainable or obtained by said method, and a pharmaceutical composition comprising such cells.

In certain embodiments, T cell maturation or differentiation in vitro may be delayed or inhibited by the method as described in WO2017070395, comprising contacting one or more T cells from a subject in need of a T cell therapy with an AKT inhibitor (such as, e.g., one or a combination of two or more AKT inhibitors disclosed in claim 8 of WO2017070395) and at least one of exogenous Interleukin-7 (IL-7) and exogenous Interleukin-15 (IL-15), wherein the resulting T cells exhibit delayed maturation or differentiation, and/or wherein the resulting T cells exhibit improved T cell function (such as, e.g., increased T cell proliferation; increased cytokine production; and/or increased cytolytic activity) relative to a T cell function of a T cell cultured in the absence of an AKT inhibitor.

In certain embodiments, a patient in need of a T cell therapy may be conditioned by a method as described in WO2016191756 comprising administering to the patient a dose of cyclophosphamide between 200 mg/m2/day and 2000 mg/m2/day and a dose of fludarabine between 20 mg/m2/day and 900 mg/m²/day.

### Genetic Modifying Agents

In certain embodiments, the one or more modulating agents may be a genetic modifying agent. The genetic modifying agent may comprise a CRISPR system, a zinc finger nuclease system, a TALEN, or a meganuclease.

In general, a CRISPR-Cas or CRISPR system as used in herein and in documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g, Shmakov et al. (2015) “Discovery and Functional Characterization of Diverse Class 2 CRISPR-Cas Systems”, Molecular Cell, DOI: dx.doi.org/10.1016/j.molcel.2015.10.008.

In certain embodiments, a protospacer adjacent motif (PAM) or PAM-like motif directs binding of the effector protein complex as disclosed herein to the target locus of interest. In some embodiments, the PAM may be a 5′ PAM (i.e., located upstream of the 5′ end of the protospacer). In other embodiments, the PAM may be a 3′ PAM (i.e., located downstream of the 5′ end of the protospacer). The term “PAM” may be used interchangeably with the term “PFS” or “protospacer flanking site” or “protospacer flanking sequence”.

In a preferred embodiment, the CRISPR effector protein may recognize a 3′ PAM. In certain embodiments, the CRISPR effector protein may recognize a 3′ PAM which is 5′H, wherein H is A, C or U.

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to a RNA polynucleotide being or comprising the target sequence. In other words, the target RNA may be a RNA polynucleotide or a part of a RNA polynucleotide to which a part of the gRNA, i.e. the guide sequence, is designed to have complementarity and to which the effector function mediated by the complex comprising CRISPR effector protein and a gRNA is to be directed. In some embodiments, a target sequence is located in the nucleus or cytoplasm of a cell.

In certain example embodiments, the CRISPR effector protein may be delivered using a nucleic acid molecule encoding the CRISPR effector protein. The nucleic acid molecule encoding a CRISPR effector protein, may advantageously be a codon optimized CRISPR effector protein. An example of a codon optimized sequence, is in this instance a sequence optimized for expression in eukaryote, e.g., humans (i.e. being optimized for expression in humans), or for another eukaryote, animal or mammal as herein discussed; see, e.g., SaCas9 human codon optimized sequence in WO 2014/093622 (PCT/US2013/074667). Whilst this is preferred, it will be appreciated that other examples are possible and codon optimization for a host species other than human, or for codon optimization for specific organs is known. In some embodiments, an enzyme coding sequence encoding a CRISPR effector protein is a codon optimized for expression in particular cells, such as eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a Cas correspond to the most frequently used codon for a particular amino acid.

In certain embodiments, the methods as described herein may comprise providing a Cas transgenic cell in which one or more nucleic acids encoding one or more guide RNAs are provided or introduced operably connected in the cell with a regulatory element comprising a promoter of one or more gene of interest. As used herein, the term “Cas transgenic cell” refers to a cell, such as a eukaryotic cell, in which a Cas gene has been genomically integrated. The nature, type, or origin of the cell are not particularly limiting according to the present invention. Also the way the Cas transgene is introduced in the cell may vary and can be any method as is known in the art. In certain embodiments, the Cas transgenic cell is obtained by introducing the Cas transgene in an isolated cell. In certain other embodiments, the Cas transgenic cell is obtained by isolating cells from a Cas transgenic organism. By means of example, and without limitation, the Cas transgenic cell as referred to herein may be derived from a Cas transgenic eukaryote, such as a Cas knock-in eukaryote. Reference is made to WO 2014/093622 (PCT/US13/74667), incorporated herein by reference. Methods of US Patent Publication Nos. 20120017290 and 20110265198 assigned to Sangamo BioSciences, Inc. directed to targeting the Rosa locus may be modified to utilize the CRISPR Cas system of the present invention. Methods of US Patent Publication No. 20130236946 assigned to Cellectis directed to targeting the Rosa locus may also be modified to utilize the CRISPR Cas system of the present invention. By means of further example reference is made to Platt et. al. (Cell; 159(2):440-455 (2014)), describing a Cas9 knock-in mouse, which is incorporated herein by reference. The Cas transgene can further comprise a Lox-Stop-polyA-Lox(LSL) cassette thereby rendering Cas expression inducible by Cre recombinase. Alternatively, the Cas transgenic cell may be obtained by introducing the Cas transgene in an isolated cell. Delivery systems for transgenes are well known in the art. By means of example, the Cas transgene may be delivered in for instance eukaryotic cell by means of vector (e.g., AAV, adenovirus, lentivirus) and/or particle and/or nanoparticle delivery, as also described herein elsewhere.

It will be understood by the skilled person that the cell, such as the Cas transgenic cell, as referred to herein may comprise further genomic alterations besides having an integrated Cas gene or the mutations arising from the sequence specific action of Cas when complexed with RNA capable of guiding Cas to a target locus.

In certain aspects the invention involves vectors, e.g. for delivering or introducing in a cell Cas and/or RNA capable of guiding Cas to a target locus (i.e. guide RNA), but also for propagating these components (e.g. in prokaryotic cells). A used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). With regards to recombination and cloning methods, mention is made of U.S. patent application Ser. No. 10/815,730, published Sep. 2, 2004 as US 2004-0171156 A1, the contents of which are herein incorporated by reference in their entirety. Thus, the embodiments disclosed herein may also comprise transgenic cells comprising the CRISPR effector system. In certain example embodiments, the transgenic cell may function as an individual discrete volume. In other words samples comprising a masking construct may be delivered to a cell, for example in a suitable delivery vesicle and if the target is present in the delivery vesicle the CRISPR effector is activated and a detectable signal generated.

The vector(s) can include the regulatory element(s), e.g., promoter(s). The vector(s) can comprise Cas encoding sequences, and/or a single, but possibly also can comprise at least 3 or 8 or 16 or 32 or 48 or 50 guide RNA(s) (e.g., sgRNAs) encoding sequences, such as 1-2, 1-3, 1-4 1-5, 3-6, 3-7, 3-8, 3-9, 3-10, 3-8, 3-16, 3-30, 3-32, 3-48, 3-50 RNA(s) (e.g., sgRNAs). In a single vector there can be a promoter for each RNA (e.g., sgRNA), advantageously when there are up to about 16 RNA(s); and, when a single vector provides for more than 16 RNA(s), one or more promoter(s) can drive expression of more than one of the RNA(s), e.g., when there are 32 RNA(s), each promoter can drive expression of two RNA(s), and when there are 48 RNA(s), each promoter can drive expression of three RNA(s). By simple arithmetic and well established cloning protocols and the teachings in this disclosure one skilled in the art can readily practice the invention as to the RNA(s) for a suitable exemplary vector such as AAV, and a suitable promoter such as the U6 promoter. For example, the packaging limit of AAV is ˜4.7 kb. The length of a single U6-gRNA (plus restriction sites for cloning) is 361 bp. Therefore, the skilled person can readily fit about 12-16, e.g., 13 U6-gRNA cassettes in a single vector. This can be assembled by any suitable means, such as a golden gate strategy used for TALE assembly (genome-engineering.org/taleffectors/). The skilled person can also use a tandem guide strategy to increase the number of U6-gRNAs by approximately 1.5 times, e.g., to increase from 12-16, e.g., 13 to approximately 18-24, e.g., about 19 U6-gRNAs. Therefore, one skilled in the art can readily reach approximately 18-24, e.g., about 19 promoter-RNAs, e.g., U6-gRNAs in a single vector, e.g., an AAV vector. A further means for increasing the number of promoters and RNAs in a vector is to use a single promoter (e.g., U6) to express an array of RNAs separated by cleavable sequences. And an even further means for increasing the number of promoter-RNAs in a vector, is to express an array of promoter-RNAs separated by cleavable sequences in the intron of a coding sequence or gene; and, in this instance it is advantageous to use a polymerase II promoter, which can have increased expression and enable the transcription of long RNA in a tissue specific manner. (see, e.g., nar.oxfordjournals.org/content/34/7/e53.short and nature.com/mt/journal/v16/n9/abs/mt2008144a.html). In an advantageous embodiment, AAV may package U6 tandem gRNA targeting up to about 50 genes. Accordingly, from the knowledge in the art and the teachings in this disclosure the skilled person can readily make and use vector(s), e.g., a single vector, expressing multiple RNAs or guides under the control or operatively or functionally linked to one or more promoters-especially as to the numbers of RNAs or guides discussed herein, without any undue experimentation.

The guide RNA(s) encoding sequences and/or Cas encoding sequences, can be functionally or operatively linked to regulatory element(s) and hence the regulatory element(s) drive expression. The promoter(s) can be constitutive promoter(s) and/or conditional promoter(s) and/or inducible promoter(s) and/or tissue specific promoter(s). The promoter can be selected from the group consisting of RNA polymerases, pol I, pol II, pol III, T7, U6, H1, retroviral Rous sarcoma virus (RSV) LTR promoter, the cytomegalovirus (CMV) promoter, the SV40 promoter, the dihydrofolate reductase promoter, the 3-actin promoter, the phosphoglycerol kinase (PGK) promoter, and the EF1α promoter. An advantageous promoter is the promoter is U6.

Additional effectors for use according to the invention can be identified by their proximity to cas1 genes, for example, though not limited to, within the region 20 kb from the start of the cas1 gene and 20 kb from the end of the cas1 gene. In certain embodiments, the effector protein comprises at least one HEPN domain and at least 500 amino acids, and wherein the C2c2 effector protein is naturally present in a prokaryotic genome within 20 kb upstream or downstream of a Cas gene or a CRISPR array. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. In certain example embodiments, the C2c2 effector protein is naturally present in a prokaryotic genome within 20 kb upstream or downstream of a Cas 1 gene. The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of. Homologous proteins may but need not be structurally related, or are only partially structurally related. An “orthologue” of a protein as used herein is a protein of a different species which performs the same or a similar function as the protein it is an orthologue of Orthologous proteins may but need not be structurally related, or are only partially structurally related.

### Guide Molecules

The methods described herein may be used to screen inhibition of CRISPR systems employing different types of guide molecules. As used herein, the term “guide sequence” and “guide molecule” in the context of a CRISPR-Cas system, comprises any polynucleotide sequence having sufficient complementarity with a target nucleic acid sequence to hybridize with the target nucleic acid sequence and direct sequence-specific binding of a nucleic acid-targeting complex to the target nucleic acid sequence. The guide sequences made using the methods disclosed herein may be a full-length guide sequence, a truncated guide sequence, a full-length sgRNA sequence, a truncated sgRNA sequence, or an E+F sgRNA sequence. In some embodiments, the degree of complementarity of the guide sequence to a given target sequence, when optimally aligned using a suitable alignment algorithm, is about or more than about 50%, 60%, 75%, 80%, 85%, 90%, 95%, 97.5%, 99%, or more. In certain example embodiments, the guide molecule comprises a guide sequence that may be designed to have at least one mismatch with the target sequence, such that a RNA duplex formed between the guide sequence and the target sequence. Accordingly, the degree of complementarity is preferably less than 99%. For instance, where the guide sequence consists of 24 nucleotides, the degree of complementarity is more particularly about 96% or less. In particular embodiments, the guide sequence is designed to have a stretch of two or more adjacent mismatching nucleotides, such that the degree of complementarity over the entire guide sequence is further reduced. For instance, where the guide sequence consists of 24 nucleotides, the degree of complementarity is more particularly about 96% or less, more particularly, about 92% or less, more particularly about 88% or less, more particularly about 84% or less, more particularly about 80% or less, more particularly about 76% or less, more particularly about 72% or less, depending on whether the stretch of two or more mismatching nucleotides encompasses 2, 3, 4, 5, 6 or 7 nucleotides, etc. In some embodiments, aside from the stretch of one or more mismatching nucleotides, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence (or a sequence in the vicinity thereof) may be evaluated in a test tube by providing the target nucleic acid sequence, components of a nucleic acid-targeting complex, including the guide sequence to be tested and a control guide sequence different from the test guide sequence, and comparing binding or rate of cleavage at or in the vicinity of the target sequence between the test and control guide sequence reactions. Other assays are possible, and will occur to those skilled in the art. A guide sequence, and hence a nucleic acid-targeting guide RNA may be selected to target any target nucleic acid sequence.

In certain embodiments, the guide sequence or spacer length of the guide molecules is from 15 to 50 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer. In certain example embodiment, the guide sequence is 15, 16, 17, 18, 19, 20, 21, 22, 23, 24, 25, 26, 27, 28, 29, 30, 31, 32, 33, 34, 35, 36, 37, 38, 39 40, 41, 42, 43, 44, 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nt.

In some embodiments, the guide sequence is an RNA sequence of between 10 to 50 nt in length, but more particularly of about 20-30 nt advantageously about 20 nt, 23-25 nt or 24 nt. The guide sequence is selected so as to ensure that it hybridizes to the target sequence. This is described more in detail below. Selection can encompass further steps which increase efficacy and specificity.

In some embodiments, the guide sequence has a canonical length (e.g., about 15-30 nt) is used to hybridize with the target RNA or DNA. In some embodiments, a guide molecule is longer than the canonical length (e.g., >30 nt) is used to hybridize with the target RNA or DNA, such that a region of the guide sequence hybridizes with a region of the RNA or DNA strand outside of the Cas-guide target complex. This can be of interest where additional modifications, such deamination of nucleotides is of interest. In alternative embodiments, it is of interest to maintain the limitation of the canonical guide sequence length.

In some embodiments, the sequence of the guide molecule (direct repeat and/or spacer) is selected to reduce the degree secondary structure within the guide molecule. In some embodiments, about or less than about 75%, 50%, 40%, 30%, 25%, 20%, 15%, 10%, 5%, 1%, or fewer of the nucleotides of the nucleic acid-targeting guide RNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A.R. Gruber et al., 2008, Cell 106(1): 23-24; and PA Carr and GM Church, 2009, Nature Biotechnology 27(12): 1151-62).

In some embodiments, it is of interest to reduce the susceptibility of the guide molecule to RNA cleavage, such as to cleavage by Cas13. Accordingly, in particular embodiments, the guide molecule is adjusted to avoid cleavage by Cas13 or other RNA-cleaving enzymes.

In certain embodiments, the guide molecule comprises non-naturally occurring nucleic acids and/or non-naturally occurring nucleotides and/or nucleotide analogs, and/or chemically modifications. Preferably, these non-naturally occurring nucleic acids and non-naturally occurring nucleotides are located outside the guide sequence. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the invention, a guide nucleic acid comprises ribonucleotides and non-ribonucleotides. In one such embodiment, a guide comprises one or more ribonucleotides and one or more deoxyribonucleotides. In an embodiment of the invention, the guide comprises one or more non-naturally occurring nucleotide or nucleotide analog such as a nucleotide with phosphorothioate linkage, a locked nucleic acid (LNA) nucleotides comprising a methylene bridge between the 2′ and 4′ carbons of the ribose ring, or bridged nucleic acids (BNA). Other examples of modified nucleotides include 2′-O-methyl analogs, 2′-deoxy analogs, or 2′-fluoro analogs. Further examples of modified bases include, but are not limited to, 2-aminopurine, 5-bromo-uridine, pseudouridine, inosine, 7-methylguanosine. Examples of guide RNA chemical modifications include, without limitation, incorporation of 2′-O-methyl (M), 2′-O-methyl 3′ phosphorothioate (MS), S-constrained ethyl(cEt), or 2′-O-methyl 3′ thioPACE (MSP) at one or more terminal nucleotides. Such chemically modified guides can comprise increased stability and increased activity as compared to unmodified guides, though on-target vs. off-target specificity is not predictable. (See, Hendel, 2015, Nat Biotechnol. 33(9):985-9, doi: 10.1038/nbt.3290, published online 29 Jun. 2015 Ragdarm et al., 0215, PNAS, E7110-E7111; Allerson et al., J. Med Chem. 2005, 48:901-904; Bramsen et al., Front. Genet., 2012, 3:154; Deng et al., PNAS, 2015, 112:11870-11875; Sharma et al., MedChemComm., 2014, 5:1454-1471; Hendel et al., Nat. Biotechnol. (2015) 33(9): 985-989; Li et al., Nature Biomedical Engineering, 2017, 1, 0066 DOI:10.1038/s41551-017-0066). In some embodiments, the 5′ and/or 3′ end of a guide RNA is modified by a variety of functional moieties including fluorescent dyes, polyethylene glycol, cholesterol, proteins, or detection tags. (See Kelly et al., 2016, J. Biotech. 233:74-83). In certain embodiments, a guide comprises ribonucleotides in a region that binds to a target RNA and one or more deoxyribonucletides and/or nucleotide analogs in a region that binds to Cas13. In an embodiment of the invention, deoxyribonucleotides and/or nucleotide analogs are incorporated in engineered guide structures, such as, without limitation, stem-loop regions, and the seed region. For Cas13 guide, in certain embodiments, the modification is not in the 5′-handle of the stem-loop regions. Chemical modification in the 5′-handle of the stem-loop region of a guide may abolish its function (see Li, et al., Nature Biomedical Engineering, 2017, 1:0066). In certain embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or 75 nucleotides of a guide is chemically modified. In some embodiments, 3-5 nucleotides at either the 3′ or the 5′ end of a guide is chemically modified. In some embodiments, only minor modifications are introduced in the seed region, such as 2′-F modifications. In some embodiments, 2′-F modification is introduced at the 3′ end of a guide. In certain embodiments, three to five nucleotides at the 5′ and/or the 3′ end of the guide are chemicially modified with 2′-O-methyl (M), 2′-O-methyl 3′ phosphorothioate (MS), S-constrained ethyl(cEt), or 2′-O-methyl 3′ thioPACE (MSP). Such modification can enhance genome editing efficiency (see Hendel et al., Nat. Biotechnol. (2015) 33(9): 985-989). In certain embodiments, all of the phosphodiester bonds of a guide are substituted with phosphorothioates (PS) for enhancing levels of gene disruption. In certain embodiments, more than five nucleotides at the 5′ and/or the 3′ end of the guide are chemicially modified with 2′-O-Me, 2′-F or S-constrained ethyl(cEt). Such chemically modified guide can mediate enhanced levels of gene disruption (see Ragdarm et al., 0215, PNAS, E7110-E7111). In an embodiment of the invention, a guide is modified to comprise a chemical moiety at its 3′ and/or 5′ end. Such moieties include, but are not limited to amine, azide, alkyne, thio, dibenzocyclooctyne (DBCO), or Rhodamine. In certain embodiment, the chemical moiety is conjugated to the guide by a linker, such as an alkyl chain. In certain embodiments, the chemical moiety of the modified guide can be used to attach the guide to another molecule, such as DNA, RNA, protein, or nanoparticles. Such chemically modified guide can be used to identify or enrich cells generically edited by a CRISPR system (see Lee et al., eLife, 2017, 6:e25312, DOI:10.7554).

In some embodiments, the modification to the guide is a chemical modification, an insertion, a deletion or a split. In some embodiments, the chemical modification includes, but is not limited to, incorporation of 2′-O-methyl (M) analogs, 2′-deoxy analogs, 2-thiouridine analogs, N6-methyladenosine analogs, 2′-fluoro analogs, 2-aminopurine, 5-bromo-uridine, pseudouridine (Ψ), N1-methylpseudouridine (melΨ), 5-methoxyuridine(5moU), inosine, 7-methylguanosine, 2′-O-methyl 3′phosphorothioate (MS), S-constrained ethyl(cEt), phosphorothioate (PS), or 2′-O-methyl 3′thioPACE (MSP). In some embodiments, the guide comprises one or more of phosphorothioate modifications. In certain embodiments, at least 1, 2, 3, 4, 5, 6, 7, 8, 9, 10, 11, 12, 13, 14, 15, 16, 17, 18, 19, 20, or 25 nucleotides of the guide are chemically modified. In certain embodiments, one or more nucleotides in the seed region are chemically modified. In certain embodiments, one or more nucleotides in the 3′-terminus are chemically modified. In certain embodiments, none of the nucleotides in the 5′-handle is chemically modified. In some embodiments, the chemical modification in the seed region is a minor modification, such as incorporation of a 2′-fluoro analog. In a specific embodiment, one nucleotide of the seed region is replaced with a 2′-fluoro analog. In some embodiments, 5 to 10 nucleotides in the 3′-terminus are chemically modified. Such chemical modifications at the 3′-terminus of the Cas13 CrRNA may improve Cas13 activity. In a specific embodiment, 1, 2, 3, 4, 5, 6, 7, 8, 9 or 10 nucleotides in the 3′-terminus are replaced with 2′-fluoro analogues. In a specific embodiment, 1, 2, 3, 4, 5, 6, 7, 8, 9 or 10 nucleotides in the 3′-terminus are replaced with 2′-O-methyl (M) analogs.

In some embodiments, the loop of the 5′-handle of the guide is modified. In some embodiments, the loop of the 5′-handle of the guide is modified to have a deletion, an insertion, a split, or chemical modifications. In certain embodiments, the modified loop comprises 3, 4, or 5 nucleotides. In certain embodiments, the loop comprises the sequence of UCUU, UUUU, UAUU, or UGUU.

In some embodiments, the guide molecule forms a stemloop with a separate non-covalently linked sequence, which can be DNA or RNA. In particular embodiments, the sequences forming the guide are first synthesized using the standard phosphoramidite synthetic protocol (Herdewijn, P., ed., Methods in Molecular Biology Col 288, Oligonucleotide Synthesis: Methods and Applications, Humana Press, New Jersey (2012)). In some embodiments, these sequences can be functionalized to contain an appropriate functional group for ligation using the standard protocol known in the art (Hermanson, G. T., Bioconjugate Techniques, Academic Press (2013)). Examples of functional groups include, but are not limited to, hydroxyl, amine, carboxylic acid, carboxylic acid halide, carboxylic acid active ester, aldehyde, carbonyl, chlorocarbonyl, imidazolylcarbonyl, hydrozide, semicarbazide, thio semicarbazide, thiol, maleimide, haloalkyl, sulfonyl, ally, propargyl, diene, alkyne, and azide. Once this sequence is functionalized, a covalent chemical bond or linkage can be formed between this sequence and the direct repeat sequence. Examples of chemical bonds include, but are not limited to, those based on carbamates, ethers, esters, amides, imines, amidines, aminotrizines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In some embodiments, these stem-loop forming sequences can be chemically synthesized. In some embodiments, the chemical synthesis uses automated, solid-phase oligonucleotide synthesis machines with 2′-acetoxyethyl orthoester (2′-ACE) (Scaringe et al., J. Am. Chem. Soc. (1998) 120: 11820-11821; Scaringe, Methods Enzymol. (2000) 317: 3-18) or 2′-thionocarbamate (2′-TC) chemistry (Dellinger et al., J. Am. Chem. Soc. (2011) 133: 11540-11546; Hendel et al., Nat. Biotechnol. (2015) 33:985-989).

In certain embodiments, the guide molecule comprises (1) a guide sequence capable of hybridizing to a target locus and (2) a tracr mate or direct repeat sequence whereby the direct repeat sequence is located upstream (i.e., 5′) from the guide sequence. In a particular embodiment the seed sequence (i.e. the sequence essential critical for recognition and/or hybridization to the sequence at the target locus) of th guide sequence is approximately within the first 10 nucleotides of the guide sequence.

In a particular embodiment the guide molecule comprises a guide sequence linked to a direct repeat sequence, wherein the direct repeat sequence comprises one or more stem loops or optimized secondary structures. In particular embodiments, the direct repeat has a minimum length of 16 nts and a single stem loop. In further embodiments the direct repeat has a length longer than 16 nts, preferably more than 17 nts, and has more than one stem loops or optimized secondary structures. In particular embodiments the guide molecule comprises or consists of the guide sequence linked to all or part of the natural direct repeat sequence. A typical Type V or Type VI CRISPR-cas guide molecule comprises (in 3′ to 5′ direction or in 5′ to 3′ direction): a guide sequence a first complimentary stretch (the “repeat”), a loop (which is typically 4 or 5 nucleotides long), a second complimentary stretch (the “anti-repeat” being complimentary to the repeat), and a poly A (often poly U in RNA) tail (terminator). In certain embodiments, the direct repeat sequence retains its natural architecture and forms a single stem loop. In particular embodiments, certain aspects of the guide architecture can be modified, for example by addition, subtraction, or substitution of features, whereas certain other aspects of guide architecture are maintained. Preferred locations for engineered guide molecule modifications, including but not limited to insertions, deletions, and substitutions include guide termini and regions of the guide molecule that are exposed when complexed with the CRISPR-Cas protein and/or target, for example the stemloop of the direct repeat sequence.

In particular embodiments, the stem comprises at least about 4 bp comprising complementary X and Y sequences, although stems of more, e.g., 5, 6, 7, 8, 9, 10, 11 or 12 or fewer, e.g., 3, 2, base pairs are also contemplated. Thus, for example X2-10 and Y2-10 (wherein X and Y represent any complementary set of nucleotides) may be contemplated. In one aspect, the stem made of the X and Y nucleotides, together with the loop will form a complete hairpin in the overall secondary structure; and, this may be advantageous and the amount of base pairs can be any amount that forms a complete hairpin. In one aspect, any complementary X:Y basepairing sequence (e.g., as to length) is tolerated, so long as the secondary structure of the entire guide molecule is preserved. In one aspect, the loop that connects the stem made of X:Y basepairs can be any sequence of the same length (e.g., 4 or 5 nucleotides) or longer that does not interrupt the overall secondary structure of the guide molecule. In one aspect, the stemloop can further comprise, e.g. an MS2 aptamer. In one aspect, the stem comprises about 5-7 bp comprising complementary X and Y sequences, although stems of more or fewer basepairs are also contemplated. In one aspect, non-Watson Crick basepairing is contemplated, where such pairing otherwise generally preserves the architecture of the stemloop at that position.

In particular embodiments the natural hairpin or stemloop structure of the guide molecule is extended or replaced by an extended stemloop. It has been demonstrated that extension of the stem can enhance the assembly of the guide molecule with the CRISPR-Cas protein (Chen et al. Cell. (2013); 155(7): 1479-1491). In particular embodiments the stem of the stemloop is extended by at least 1, 2, 3, 4, 5 or more complementary basepairs (i.e. corresponding to the addition of 2, 4, 6, 8, 10 or more nucleotides in the guide molecule). In particular embodiments these are located at the end of the stem, adjacent to the loop of the stemloop.

In particular embodiments, the susceptibility of the guide molecule to RNAses or to decreased expression can be reduced by slight modifications of the sequence of the guide molecule which do not affect its function. For instance, in particular embodiments, premature termination of transcription, such as premature transcription of U6 Pol-III, can be removed by modifying a putative Pol-III terminator (4 consecutive U's) in the guide molecules sequence. Where such sequence modification is required in the stemloop of the guide molecule, it is preferably ensured by a basepair flip.

In a particular embodiment, the direct repeat may be modified to comprise one or more protein-binding RNA aptamers. In a particular embodiment, one or more aptamers may be included such as part of optimized secondary structure. Such aptamers may be capable of binding a bacteriophage coat protein as detailed further herein.

In some embodiments, the guide molecule forms a duplex with a target RNA comprising at least one target cytosine residue to be edited. Upon hybridization of the guide RNA molecule to the target RNA, the cytidine deaminase binds to the single strand RNA in the duplex made accessible by the mismatch in the guide sequence and catalyzes deamination of one or more target cytosine residues comprised within the stretch of mismatching nucleotides.

A guide sequence, and hence a nucleic acid-targeting guide RNA may be selected to target any target nucleic acid sequence. The target sequence may be mRNA.

In certain embodiments, the target sequence should be associated with a PAM (protospacer adjacent motif) or PFS (protospacer flanking sequence or site); that is, a short sequence recognized by the CRISPR complex. Depending on the nature of the CRISPR-Cas protein, the target sequence should be selected such that its complementary sequence in the DNA duplex (also referred to herein as the non-target sequence) is upstream or downstream of the PAM. In the embodiments of the present invention where the CRISPR-Cas protein is a Cas13 protein, the complementary sequence of the target sequence is downstream or 3′ of the PAM or upstream or 5′ of the PAM. The precise sequence and length requirements for the PAM differ depending on the Cas13 protein used, but PAMs are typically 2-5 base pair sequences adjacent the protospacer (that is, the target sequence). Examples of the natural PAM sequences for different Cas13 orthologues are provided herein below and the skilled person will be able to identify further PAM sequences for use with a given Cas13 protein.

Further, engineering of the PAM Interacting (PI) domain may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously.

In particular embodiment, the guide is an escorted guide. By “escorted” is meant that the CRISPR-Cas system or complex or guide is delivered to a selected time or place within a cell, so that activity of the CRISPR-Cas system or complex or guide is spatially or temporally controlled. For example, the activity and destination of the 3 CRISPR-Cas system or complex or guide may be controlled by an escort RNA aptamer sequence that has binding affinity for an aptamer ligand, such as a cell surface protein or other localized cellular component. Alternatively, the escort aptamer may for example be responsive to an aptamer effector on or in the cell, such as a transient effector, such as an external energy source that is applied to the cell at a particular time.

The escorted CRISPR-Cas systems or complexes have a guide molecule with a functional structure designed to improve guide molecule structure, architecture, stability, genetic expression, or any combination thereof. Such a structure can include an aptamer.

Aptamers are biomolecules that can be designed or selected to bind tightly to other ligands, for example using a technique called systematic evolution of ligands by exponential enrichment (SELEX; Tuerk C, Gold L: “Systematic evolution of ligands by exponential enrichment: RNA ligands to bacteriophage T4 DNA polymerase.” Science 1990, 249:505-510). Nucleic acid aptamers can for example be selected from pools of random-sequence oligonucleotides, with high binding affinities and specificities for a wide range of biomedically relevant targets, suggesting a wide range of therapeutic utilities for aptamers (Keefe, Anthony D., Supriya Pai, and Andrew Ellington. “Aptamers as therapeutics.” Nature Reviews Drug Discovery 9.7 (2010): 537-550). These characteristics also suggest a wide range of uses for aptamers as drug delivery vehicles (Levy-Nissenbaum, Etgar, et al. “Nanotechnology and aptamers: applications in drug delivery.” Trends in biotechnology 26.8 (2008): 442-449; and, Hicke B J, Stephens A W. “Escort aptamers: a delivery service for diagnosis and therapy.” J Clin Invest 2000, 106:923-928.). Aptamers may also be constructed that function as molecular switches, responding to a que by changing properties, such as RNA aptamers that bind fluorophores to mimic the activity of green flourescent protein (Paige, Jeremy S., Karen Y. Wu, and Samie R. Jaffrey. “RNA mimics of green fluorescent protein.” Science 333.6042 (2011): 642-646). It has also been suggested that aptamers may be used as components of targeted siRNA therapeutic delivery systems, for example targeting cell surface proteins (Zhou, Jiehua, and John J. Rossi. “Aptamer-targeted cell-specific RNA interference.” Silence 1.1 (2010): 4).

Accordingly, in particular embodiments, the guide molecule is modified, e.g., by one or more aptamer(s) designed to improve guide molecule delivery, including delivery across the cellular membrane, to intracellular compartments, or into the nucleus. Such a structure can include, either in addition to the one or more aptamer(s) or without such one or more aptamer(s), moiety(ies) so as to render the guide molecule deliverable, inducible or responsive to a selected effector. The invention accordingly comprehends an guide molecule that responds to normal or pathological physiological conditions, including without limitation pH, hypoxia, O₂ concentration, temperature, protein concentration, enzymatic concentration, lipid structure, light exposure, mechanical disruption (e.g. ultrasound waves), magnetic fields, electric fields, or electromagnetic radiation.

Light responsiveness of an inducible system may be achieved via the activation and binding of cryptochrome-2 and CIB1. Blue light stimulation induces an activating conformational change in cryptochrome-2, resulting in recruitment of its binding partner CIB1. This binding is fast and reversible, achieving saturation in <15 sec following pulsed stimulation and returning to baseline <15 min after the end of stimulation. These rapid binding kinetics result in a system temporally bound only by the speed of transcription/translation and transcript/protein degradation, rather than uptake and clearance of inducing agents. Crytochrome-2 activation is also highly sensitive, allowing for the use of low light intensity stimulation and mitigating the risks of phototoxicity. Further, in a context such as the intact mammalian brain, variable light intensity may be used to control the size of a stimulated region, allowing for greater precision than vector delivery alone may offer.

The invention contemplates energy sources such as electromagnetic radiation, sound energy or thermal energy to induce the guide. Advantageously, the electromagnetic radiation is a component of visible light. In a preferred embodiment, the light is a blue light with a wavelength of about 450 to about 495 nm. In an especially preferred embodiment, the wavelength is about 488 nm. In another preferred embodiment, the light stimulation is via pulses. The light power may range from about 0-9 mW/cm². In a preferred embodiment, a stimulation paradigm of as low as 0.25 sec every 15 sec should result in maximal activation.

The chemical or energy sensitive guide may undergo a conformational change upon induction by the binding of a chemical source or by the energy allowing it act as a guide and have the Cas13 CRISPR-Cas system or complex function. The invention can involve applying the chemical source or energy so as to have the guide function and the Cas13 CRISPR-Cas system or complex function; and optionally further determining that the expression of the genomic locus is altered.

There are several different designs of this chemical inducible system: 1. ABI-PYL based system inducible by Abscisic Acid (ABA) (see, e.g., stke.sciencemag.org/cgi/content/abstract/sigtrans; 4/164/rs2), 2. FKBP-FRB based system inducible by rapamycin (or related chemicals based on rapamycin) (see, e.g., www.nature.com/nmeth/journal/v2/n6/full/nmeth763.html), 3. GID1-GAI based system inducible by Gibberellin (GA) (see, e.g., www.nature.com/nchembio/journal/v8/n5/full/nchembio.922.html).

A chemical inducible system can be an estrogen receptor (ER) based system inducible by 4-hydroxytamoxifen (4OHT) (see, e.g., www.pnas.org/content/104/3/1027.abstract). A mutated ligand-binding domain of the estrogen receptor called ERT2 translocates into the nucleus of cells upon binding of 4-hydroxytamoxifen. In further embodiments of the invention any naturally occurring or engineered derivative of any nuclear receptor, thyroid hormone receptor, retinoic acid receptor, estrogren receptor, estrogen-related receptor, glucocorticoid receptor, progesterone receptor, androgen receptor may be used in inducible systems analogous to the ER based inducible system.

Another inducible system is based on the design using Transient receptor potential (TRP) ion channel based system inducible by energy, heat or radio-wave (see, e.g., www.sciencemag.org/content/336/6081/604). These TRP family proteins respond to different stimuli, including light and heat. When this protein is activated by light or heat, the ion channel will open and allow the entering of ions such as calcium into the plasma membrane. This influx of ions will bind to intracellular ion interacting partners linked to a polypeptide including the guide and the other components of the Cas13 CRISPR-Cas complex or system, and the binding will induce the change of sub-cellular localization of the polypeptide, leading to the entire polypeptide entering the nucleus of cells. Once inside the nucleus, the guide protein and the other components of the Cas13 CRISPR-Cas complex will be active and modulating target gene expression in cells.

While light activation may be an advantageous embodiment, sometimes it may be disadvantageous especially for in vivo applications in which the light may not penetrate the skin or other organs. In this instance, other methods of energy activation are contemplated, in particular, electric field energy and/or ultrasound which have a similar effect.

Electric field energy is preferably administered substantially as described in the art, using one or more electric pulses of from about 1 Volt/cm to about 10 kVolts/cm under in vivo conditions. Instead of or in addition to the pulses, the electric field may be delivered in a continuous manner. The electric pulse may be applied for between 1 μs and 500 milliseconds, preferably between 1 μs and 100 milliseconds. The electric field may be applied continuously or in a pulsed manner for 5 about minutes.

As used herein, ‘electric field energy’ is the electrical energy to which a cell is exposed. Preferably the electric field has a strength of from about 1 Volt/cm to about 10 kVolts/cm or more under in vivo conditions (see WO97/49450).

As used herein, the term “electric field” includes one or more pulses at variable capacitance and voltage and including exponential and/or square wave and/or modulated wave and/or modulated square wave forms. References to electric fields and electricity should be taken to include reference the presence of an electric potential difference in the environment of a cell. Such an environment may be set up by way of static electricity, alternating current (AC), direct current (DC), etc, as known in the art. The electric field may be uniform, non-uniform or otherwise, and may vary in strength and/or direction in a time dependent manner.

Single or multiple applications of electric field, as well as single or multiple applications of ultrasound are also possible, in any order and in any combination. The ultrasound and/or the electric field may be delivered as single or multiple continuous applications, or as pulses (pulsatile delivery).

Electroporation has been used in both in vitro and in vivo procedures to introduce foreign material into living cells. With in vitro applications, a sample of live cells is first mixed with the agent of interest and placed between electrodes such as parallel plates. Then, the electrodes apply an electrical field to the cell/implant mixture. Examples of systems that perform in vitro electroporation include the Electro Cell Manipulator ECM600 product, and the Electro Square Porator T820, both made by the BTX Division of Genetronics, Inc (see U.S. Pat. No. 5,869,326).

The known electroporation techniques (both in vitro and in vivo) function by applying a brief high voltage pulse to electrodes positioned around the treatment region. The electric field generated between the electrodes causes the cell membranes to temporarily become porous, whereupon molecules of the agent of interest enter the cells. In known electroporation applications, this electric field comprises a single square wave pulse on the order of 1000 V/cm, of about 100.mu.s duration. Such a pulse may be generated, for example, in known applications of the Electro Square Porator T820.

Preferably, the electric field has a strength of from about 1 V/cm to about 10 kV/cm under in vitro conditions. Thus, the electric field may have a strength of 1 V/cm, 2 V/cm, 3 V/cm, 4 V/cm, 5 V/cm, 6 V/cm, 7 V/cm, 8 V/cm, 9 V/cm, 10 V/cm, 20 V/cm, 50 V/cm, 100 V/cm, 200 V/cm, 300 V/cm, 400 V/cm, 500 V/cm, 600 V/cm, 700 V/cm, 800 V/cm, 900 V/cm, 1 kV/cm, 2 kV/cm, 5 kV/cm, 10 kV/cm, 20 kV/cm, 50 kV/cm or more. More preferably from about 0.5 kV/cm to about 4.0 kV/cm under in vitro conditions. Preferably the electric field has a strength of from about 1 V/cm to about 10 kV/cm under in vivo conditions. However, the electric field strengths may be lowered where the number of pulses delivered to the target site are increased. Thus, pulsatile delivery of electric fields at lower field strengths is envisaged.

Preferably the application of the electric field is in the form of multiple pulses such as double pulses of the same strength and capacitance or sequential pulses of varying strength and/or capacitance. As used herein, the term “pulse” includes one or more electric pulses at variable capacitance and voltage and including exponential and/or square wave and/or modulated wave/square wave forms.

Preferably the electric pulse is delivered as a waveform selected from an exponential wave form, a square wave form, a modulated wave form and a modulated square wave form.

A preferred embodiment employs direct current at low voltage. Thus, Applicants disclose the use of an electric field which is applied to the cell, tissue or tissue mass at a field strength of between 1V/cm and 20V/cm, for a period of 100 milliseconds or more, preferably 15 minutes or more.

Ultrasound is advantageously administered at a power level of from about 0.05 W/cm2 to about 100 W/cm2. Diagnostic or therapeutic ultrasound may be used, or combinations thereof.

As used herein, the term “ultrasound” refers to a form of energy which consists of mechanical vibrations the frequencies of which are so high they are above the range of human hearing. Lower frequency limit of the ultrasonic spectrum may generally be taken as about 20 kHz. Most diagnostic applications of ultrasound employ frequencies in the range 1 and 15 MHz′ (From Ultrasonics in Clinical Diagnosis, P. N. T. Wells, ed., 2nd. Edition, Publ. Churchill Livingstone [Edinburgh, London & NY, 1977]).

Ultrasound has been used in both diagnostic and therapeutic applications. When used as a diagnostic tool (“diagnostic ultrasound”), ultrasound is typically used in an energy density range of up to about 100 mW/cm2 (FDA recommendation), although energy densities of up to 750 mW/cm2 have been used. In physiotherapy, ultrasound is typically used as an energy source in a range up to about 3 to 4 W/cm2 (WHO recommendation). In other therapeutic applications, higher intensities of ultrasound may be employed, for example, HIFU at 100 W/cm up to 1 kW/cm2 (or even higher) for short periods of time. The term “ultrasound” as used in this specification is intended to encompass diagnostic, therapeutic and focused ultrasound.

Focused ultrasound (FUS) allows thermal energy to be delivered without an invasive probe (see Morocz et al 1998 Journal of Magnetic Resonance Imaging Vol. 8, No. 1, pp. 136-142. Another form of focused ultrasound is high intensity focused ultrasound (HIFU) which is reviewed by Moussatov et al in Ultrasonics (1998) Vol. 36, No. 8, pp. 893-900 and TranHuuHue et al in Acustica (1997) Vol. 83, No. 6, pp. 1103-1106.

Preferably, a combination of diagnostic ultrasound and a therapeutic ultrasound is employed. This combination is not intended to be limiting, however, and the skilled reader will appreciate that any variety of combinations of ultrasound may be used. Additionally, the energy density, frequency of ultrasound, and period of exposure may be varied.

Preferably the exposure to an ultrasound energy source is at a power density of from about 0.05 to about 100 Wcm-2. Even more preferably, the exposure to an ultrasound energy source is at a power density of from about 1 to about 15 Wcm-2.

Preferably the exposure to an ultrasound energy source is at a frequency of from about 0.015 to about 10.0 MHz. More preferably the exposure to an ultrasound energy source is at a frequency of from about 0.02 to about 5.0 MHz or about 6.0 MHz. Most preferably, the ultrasound is applied at a frequency of 3 MHz.

Preferably the exposure is for periods of from about 10 milliseconds to about 60 minutes. Preferably the exposure is for periods of from about 1 second to about 5 minutes. More preferably, the ultrasound is applied for about 2 minutes. Depending on the particular target cell to be disrupted, however, the exposure may be for a longer duration, for example, for 15 minutes.

Advantageously, the target tissue is exposed to an ultrasound energy source at an acoustic power density of from about 0.05 Wcm-2 to about 10 Wcm-2 with a frequency ranging from about 0.015 to about 10 MHz (see WO 98/52609). However, alternatives are also possible, for example, exposure to an ultrasound energy source at an acoustic power density of above 100 Wcm-2, but for reduced periods of time, for example, 1000 Wcm-2 for periods in the millisecond range or less.

Preferably the application of the ultrasound is in the form of multiple pulses; thus, both continuous wave and pulsed wave (pulsatile delivery of ultrasound) may be employed in any combination. For example, continuous wave ultrasound may be applied, followed by pulsed wave ultrasound, or vice versa. This may be repeated any number of times, in any order and combination. The pulsed wave ultrasound may be applied against a background of continuous wave ultrasound, and any number of pulses may be used in any number of groups.

Preferably, the ultrasound may comprise pulsed wave ultrasound. In a highly preferred embodiment, the ultrasound is applied at a power density of 0.7 Wcm-2 or 1.25 Wcm-2 as a continuous wave. Higher power densities may be employed if pulsed wave ultrasound is used.

Use of ultrasound is advantageous as, like light, it may be focused accurately on a target. Moreover, ultrasound is advantageous as it may be focused more deeply into tissues unlike light. It is therefore better suited to whole-tissue penetration (such as but not limited to a lobe of the liver) or whole organ (such as but not limited to the entire liver or an entire muscle, such as the heart) therapy. Another important advantage is that ultrasound is a non-invasive stimulus which is used in a wide variety of diagnostic and therapeutic applications. By way of example, ultrasound is well known in medical imaging techniques and, additionally, in orthopedic therapy. Furthermore, instruments suitable for the application of ultrasound to a subject vertebrate are widely available and their use is well known in the art.

In particular embodiments, the guide molecule is modified by a secondary structure to increase the specificity of the CRISPR-Cas system and the secondary structure can protect against exonuclease activity and allow for 5′ additions to the guide sequence also referred to herein as a protected guide molecule.

In one aspect, the invention provides for hybridizing a “protector RNA” to a sequence of the guide molecule, wherein the “protector RNA” is an RNA strand complementary to the 3′ end of the guide molecule to thereby generate a partially double-stranded guide RNA. In an embodiment of the invention, protecting mismatched bases (i.e. the bases of the guide molecule which do not form part of the guide sequence) with a perfectly complementary protector sequence decreases the likelihood of target RNA binding to the mismatched basepairs at the 3′ end. In particular embodiments of the invention, additional sequences comprising an extented length may also be present within the guide molecule such that the guide comprises a protector sequence within the guide molecule. This “protector sequence” ensures that the guide molecule comprises a “protected sequence” in addition to an “exposed sequence” (comprising the part of the guide sequence hybridizing to the target sequence). In particular embodiments, the guide molecule is modified by the presence of the protector guide to comprise a secondary structure such as a hairpin. Advantageously there are three or four to thirty or more, e.g., about 10 or more, contiguous base pairs having complementarity to the protected sequence, the guide sequence or both. It is advantageous that the protected portion does not impede thermodynamics of the CRISPR-Cas system interacting with its target. By providing such an extension including a partially double stranded guide molecule, the guide molecule is considered protected and results in improved specific binding of the CRISPR-Cas complex, while maintaining specific activity.

In particular embodiments, use is made of a truncated guide (tru-guide), i.e. a guide molecule which comprises a guide sequence which is truncated in length with respect to the canonical guide sequence length. As described by Nowak et al. (Nucleic Acids Res (2016) 44 (20): 9555-9564), such guides may allow catalytically active CRISPR-Cas enzyme to bind its target without cleaving the target RNA. In particular embodiments, a truncated guide is used which allows the binding of the target but retains only nickase activity of the CRISPR-Cas enzyme.

### CRISPR RNA-Targeting Effector Proteins

In one example embodiment, the CRISPR system effector protein is an RNA-targeting effector protein. In certain embodiments, the CRISPR system effector protein is a Type VI CRISPR system targeting RNA (e.g., Cas13a, Cas13b, Cas13c or Cas13d). Example RNA-targeting effector proteins include Cas13b and C2c2 (now known as Cas13a). It will be understood that the term “C2c2” herein is used interchangeably with “Cas13a”. “C2c2” is now referred to as “Cas13a”, and the terms are used interchangeably herein unless indicated otherwise. As used herein, the term “Cas13” refers to any Type VI CRISPR system targeting RNA (e.g., Cas13a, Cas13b, Cas13c or Cas13d). When the CRISPR protein is a C2c2 protein, a tracrRNA is not required. C2c2 has been described in Abudayyeh et al. (2016) “C2c2 is a single-component programmable RNA-guided RNA-targeting CRISPR effector”; Science; DOI: 10.1 126/science.aaf5573; and Shmakov et al. (2015) “Discovery and Functional Characterization of Diverse Class 2 CRISPR-Cas Systems”, Molecular Cell, DOI: dx.doi.org/10.1016/j.molcel.2015.10.008; which are incorporated herein in their entirety by reference. Cas13b has been described in Smargon et al. (2017) “Cas13b Is a Type VI-B CRISPR-Associated RNA-Guided RNases Differentially Regulated by Accessory Proteins Csx27 and Csx28,” Molecular Cell. 65, 1-13; dx.doi.org/10.1016/j.molcel.2016.12.023., which is incorporated herein in its entirety by reference.

In some embodiments, one or more elements of a nucleic acid-targeting system is derived from a particular organism comprising an endogenous CRISPR RNA-targeting system. In certain example embodiments, the effector protein CRISPR RNA-targeting system comprises at least one HEPN domain, including but not limited to the HEPN domains described herein, HEPN domains known in the art, and domains recognized to be HEPN domains by comparison to consensus sequence motifs. Several such domains are provided herein. In one non-limiting example, a consensus sequence can be derived from the sequences of C2c2 or Cas13b orthologs provided herein. In certain example embodiments, the effector protein comprises a single HEPN domain. In certain other example embodiments, the effector protein comprises two HEPN domains.

In one example embodiment, the effector protein comprise one or more HEPN domains comprising a RxxxxH motif sequence. The RxxxxH motif sequence can be, without limitation, from a HEPN domain described herein or a HEPN domain known in the art. RxxxxH motif sequences further include motif sequences created by combining portions of two or more HEPN domains. As noted, consensus sequences can be derived from the sequences of the orthologs disclosed in U.S. Provisional Patent Application 62/432,240 entitled “Novel CRISPR Enzymes and Systems,” U.S. Provisional Patent Application 62/471,710 entitled “Novel Type VI CRISPR Orthologs and Systems” filed on Mar. 15, 2017, and U.S. Provisional Patent Application entitled “Novel Type VI CRISPR Orthologs and Systems,” labeled as attorney docket number 47627-05-2133 and filed on Apr. 12, 2017.

In certain other example embodiments, the CRISPR system effector protein is a C2c2 nuclease. The activity of C2c2 may depend on the presence of two HEPN domains. These have been shown to be RNase domains, i.e. nuclease (in particular an endonuclease) cutting RNA. C2c2 HEPN may also target DNA, or potentially DNA and/or RNA. On the basis that the HEPN domains of C2c2 are at least capable of binding to and, in their wild-type form, cutting RNA, then it is preferred that the C2c2 effector protein has RNase function. Regarding C2c2 CRISPR systems, reference is made to U.S. Provisional 62/351,662 filed on Jun. 17, 2016 and U.S. Provisional 62/376,377 filed on Aug. 17, 2016. Reference is also made to U.S. Provisional 62/351,803 filed on Jun. 17, 2016. Reference is also made to U.S. Provisional entitled “Novel Crispr Enzymes and Systems” filed Dec. 8, 2016 bearing Broad Institute No. 10035.PA4 and Attorney Docket No. 47627.03.2133. Reference is further made to East-Seletsky et al. “Two distinct RNase activities of CRISPR-C2c2 enable guide-RNA processing and RNA detection” Nature doi:10/1038/nature19802 and Abudayyeh et al. “C2c2 is a single-component programmable RNA-guided RNA targeting CRISPR effector” bioRxiv doi:10.1101/054742.

In certain embodiments, the C2c2 effector protein is from an organism of a genus selected from the group consisting of: Leptotrichia, Listeria, Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, Campylobacter, and Lachnospira, or the C2c2 effector protein is an organism selected from the group consisting of: Leptotrichia shahii, Leptotrichia. wadei, Listeria seeligeri, Clostridium aminophilum, Carnobacterium gallinarum, Paludibacter propionicigenes, Listeria weihenstephanensis, or the C2c2 effector protein is a L. wadei F0279 or L. wadei F0279 (Lw2) C2C2 effector protein. In another embodiment, the one or more guide RNAs are designed to detect a single nucleotide polymorphism, splice variant of a transcript, or a frameshift mutation in a target RNA or DNA.

In certain example embodiments, the RNA-targeting effector protein is a Type VI-B effector protein, such as Cas13b and Group 29 or Group 30 proteins. In certain example embodiments, the RNA-targeting effector protein comprises one or more HEPN domains. In certain example embodiments, the RNA-targeting effector protein comprises a C-terminal HEPN domain, a N-terminal HEPN domain, or both. Regarding example Type VI-B effector proteins that may be used in the context of this invention, reference is made to U.S. application Ser. No. 15/331,792 entitled “Novel CRISPR Enzymes and Systems” and filed Oct. 21, 2016, International Patent Application No. PCT/US2016/058302 entitled “Novel CRISPR Enzymes and Systems”, and filed Oct. 21, 2016, and Smargon et al. “Cas13b is a Type VI-B CRISPR-associated RNA-Guided RNase differentially regulated by accessory proteins Csx27 and Csx28” Molecular Cell, 65, 1-13 (2017); dx.doi.org/10.1016/j.molcel.2016.12.023, and U.S. Provisional Application No. to be assigned, entitled “Novel Cas13b Orthologues CRISPR Enzymes and System” filed Mar. 15, 2017. In particular embodiments, the Cas13b enzyme is derived from Bergeyella zoohelcum.

In certain example embodiments, the RNA-targeting effector protein is a Cas13c effector protein as disclosed in U.S. Provisional Patent Application No. 62/525,165 filed Jun. 26, 2017, and PCT Application No. US 2017/047193 filed Aug. 16, 2017.

In some embodiments, one or more elements of a nucleic acid-targeting system is derived from a particular organism comprising an endogenous CRISPR RNA-targeting system. In certain embodiments, the CRISPR RNA-targeting system is found in Eubacterium and Ruminococcus. In certain embodiments, the effector protein comprises targeted and collateral ssRNA cleavage activity. In certain embodiments, the effector protein comprises dual HEPN domains. In certain embodiments, the effector protein lacks a counterpart to the Helical-1 domain of Cas13a. In certain embodiments, the effector protein is smaller than previously characterized class 2 CRISPR effectors, with a median size of 928 aa. This median size is 190 aa (17%) less than that of Cas13c, more than 200 aa (18%) less than that of Cas13b, and more than 300 aa (26%) less than that of Cas13a. In certain embodiments, the effector protein has no requirement for a flanking sequence (e.g., PFS, PAM).

In certain embodiments, the effector protein locus structures include a WYL domain containing accessory protein (so denoted after three amino acids that were conserved in the originally identified group of these domains; see, e.g., WYL domain IPR026881). In certain embodiments, the WYL domain accessory protein comprises at least one helix-turn-helix (HTH) or ribbon-helix-helix (RHH) DNA-binding domain. In certain embodiments, the WYL domain containing accessory protein increases both the targeted and the collateral ssRNA cleavage activity of the RNA-targeting effector protein. In certain embodiments, the WYL domain containing accessory protein comprises an N-terminal RHH domain, as well as a pattern of primarily hydrophobic conserved residues, including an invariant tyrosine-leucine doublet corresponding to the original WYL motif. In certain embodiments, the WYL domain containing accessory protein is WYLL. WYL1 is a single WYL-domain protein associated primarily with Ruminococcus.

In other example embodiments, the Type VI RNA-targeting Cas enzyme is Cas13d. In certain embodiments, Cas13d is Eubacterium siraeum DSM 15702 (EsCas13d) or Ruminococcus sp. N15.MGS-57 (RspCas13d) (see, e.g., Yan et al., Cas13d Is a Compact RNA-Targeting Type VI CRISPR Effector Positively Modulated by a WYL-Domain-Containing Accessory Protein, Molecular Cell (2018), doi.org/10.1016/j.molcel.2018.02.028). RspCas13d and EsCas13d have no flanking sequence requirements (e.g., PFS, PAM).

### Cas13 RNA Editing

In one aspect, the invention provides a method of modifying or editing a target transcript in a eukaryotic cell. In some embodiments, the method comprises allowing a CRISPR-Cas effector module complex to bind to the target polynucleotide to effect RNA base editing, wherein the CRISPR-Cas effector module complex comprises a Cas effector module complexed with a guide sequence hybridized to a target sequence within said target polynucleotide, wherein said guide sequence is linked to a direct repeat sequence. In some embodiments, the Cas effector module comprises a catalytically inactive CRISPR-Cas protein. In some embodiments, the guide sequence is designed to introduce one or more mismatches to the RNA/RNA duplex formed between the target sequence and the guide sequence. In particular embodiments, the mismatch is an A-C mismatch. In some embodiments, the Cas effector may associate with one or more functional domains (e.g. via fusion protein or suitable linkers). In some embodiments, the effector domain comprises one or more cytindine or adenosine deaminases that mediate endogenous editing of via hydrolytic deamination. In particular embodiments, the effector domain comprises the adenosine deaminase acting on RNA (ADAR) family of enzymes. In particular embodiments, the adenosine deaminase protein or catalytic domain thereof capable of deaminating adenosine or cytidine in RNA or is an RNA specific adenosine deaminase and/or is a bacterial, human, cephalopod, or Drosophila adenosine deaminase protein or catalytic domain thereof, preferably TadA, more preferably ADAR, optionally huADAR, optionally (hu)ADAR1 or (hu)ADAR2, preferably huADAR2 or catalytic domain thereof.

The present application relates to modifying a target RNA sequence of interest (see, e.g, Cox et al., Science. 2017 Nov. 24; 358(6366):1019-1027). Using RNA-targeting rather than DNA targeting offers several advantages relevant for therapeutic development. First, there are substantial safety benefits to targeting RNA: there will be fewer off-target events because the available sequence space in the transcriptome is significantly smaller than the genome, and if an off-target event does occur, it will be transient and less likely to induce negative side effects. Second, RNA-targeting therapeutics will be more efficient because they are cell-type independent and not have to enter the nucleus, making them easier to deliver.

A further aspect of the invention relates to the method and composition as envisaged herein for use in prophylactic or therapeutic treatment, preferably wherein said target locus of interest is within a human or animal and to methods of modifying an Adenine or Cytidine in a target RNA sequence of interest, comprising delivering to said target RNA, the composition as described herein. In particular embodiments, the CRISPR system and the adenosine deaminase, or catalytic domain thereof, are delivered as one or more polynucleotide molecules, as a ribonucleoprotein complex, optionally via particles, vesicles, or one or more viral vectors. In particular embodiments, the invention thus comprises compositions for use in therapy. This implies that the methods can be performed in vivo, ex vivo or in vitro. In particular embodiments, when the target is a human or animal target, the method is carried out ex vivo or in vitro.

A further aspect of the invention relates to the method as envisaged herein for use in prophylactic or therapeutic treatment, preferably wherein said target of interest is within a human or animal and to methods of modifying an Adenine or Cytidine in a target RNA sequence of interest, comprising delivering to said target RNA, the composition as described herein. In particular embodiments, the CRISPR system and the adenosine deaminase, or catalytic domain thereof, are delivered as one or more polynucleotide molecules, as a ribonucleoprotein complex, optionally via particles, vesicles, or one or more viral vectors.

In one aspect, the invention provides a method of generating a eukaryotic cell comprising a modified or edited gene. In some embodiments, the method comprises (a) introducing one or more vectors into a eukaryotic cell, wherein the one or more vectors drive expression of one or more of: Cas effector module, and a guide sequence linked to a direct repeat sequence, wherein the Cas effector module associate one or more effector domains that mediate base editing, and (b) allowing a CRISPR-Cas effector module complex to bind to a target polynucleotide to effect base editing of the target polynucleotide within said disease gene, wherein the CRISPR-Cas effector module complex comprises a Cas effector module complexed with the guide sequence that is hybridized to the target sequence within the target polynucleotide, wherein the guide sequence may be designed to introduce one or more mismatches between the RNA/RNA duplex formed between the guide sequence and the target sequence. In particular embodiments, the mismatch is an A-C mismatch. In some embodiments, the Cas effector may associate with one or more functional domains (e.g. via fusion protein or suitable linkers). In some embodiments, the effector domain comprises one or more cytidine or adenosine deaminases that mediate endogenous editing of via hydrolytic deamination. In particular embodiments, the effector domain comprises the adenosine deaminase acting on RNA (ADAR) family of enzymes. In particular embodiments, the adenosine deaminase protein or catalytic domain thereof capable of deaminating adenosine or cytidine in RNA or is an RNA specific adenosine deaminase and/or is a bacterial, human, cephalopod, or Drosophila adenosine deaminase protein or catalytic domain thereof, preferably TadA, more preferably ADAR, optionally huADAR, optionally (hu)ADAR1 or (hu)ADAR2, preferably huADAR2 or catalytic domain thereof.

A further aspect relates to an isolated cell obtained or obtainable from the methods described herein comprising the composition described herein or progeny of said modified cell, preferably wherein said cell comprises a hypoxanthine or a guanine in replace of said Adenine in said target RNA of interest compared to a corresponding cell not subjected to the method. In particular embodiments, the cell is a eukaryotic cell, preferably a human or non-human animal cell, optionally a therapeutic T cell or an antibody-producing B-cell.

In some embodiments, the modified cell is a therapeutic T cell, such as a T cell suitable for adoptive cell transfer therapies (e.g., CAR-T therapies). The modification may result in one or more desirable traits in the therapeutic T cell, as described further herein.

The invention further relates to a method for cell therapy, comprising administering to a patient in need thereof the modified cell described herein, wherein the presence of the modified cell remedies a disease in the patient.

The present invention may be further illustrated and extended based on aspects of CRISPR-Cas development and use as set forth in the following articles and particularly as relates to delivery of a CRISPR protein complex and uses of an RNA guided endonuclease in cells and organisms:
Multiplex genome engineering using CRISPR-Cas systems. Cong, L., Ran, F. A., Cox, D., Lin, S., Barretto, R., Habib, N., Hsu, P. D., Wu, X., Jiang, W., Marraffini, L. A., & Zhang, F. Science February 15; 339(6121):819-23 (2013);
RNA-guided editing of bacterial genomes using CRISPR-Cas systems. Jiang W., Bikard D., Cox D., Zhang F, Marraffini L A. Nat Biotechnol March; 31(3):233-9 (2013);
One-Step Generation of Mice Carrying Mutations in Multiple Genes by CRISPR-Cas-Mediated Genome Engineering. Wang H., Yang H., Shivalila C S., Dawlaty M M., Cheng A W., Zhang F., Jaenisch R. Cell May 9; 153(4):910-8 (2013);
Optical control of mammalian endogenous transcription and epigenetic states. Konermann S, Brigham M D, Trevino A E, Hsu P D, Heidenreich M, Cong L, Platt R J, Scott D A, Church G M, Zhang F. Nature. August 22; 500(7463):472-6. doi: 10.1038/Nature12466. Epub 2013 Aug. 23 (2013);
Double Nicking by RNA-Guided CRISPR Cas9 for Enhanced Genome Editing Specificity. Ran, F A., Hsu, P D., Lin, C Y., Gootenberg, J S., Konermann, S., Trevino, A E., Scott, D A., Inoue, A., Matoba, S., Zhang, Y., & Zhang, F. Cell August 28. pii: S0092-8674(13)01015-5 (2013-A);
DNA targeting specificity of RNA-guided Cas9 nucleases. Hsu, P., Scott, D., Weinstein, J., Ran, F A., Konermann, S., Agarwala, V., Li, Y., Fine, E., Wu, X., Shalem, O., Cradick, T J., Marraffini, L A., Bao, G., & Zhang, F. Nat Biotechnol doi:10.1038/nbt.2647 (2013);
Genome engineering using the CRISPR-Cas9 system. Ran, F A., Hsu, P D., Wright, J., Agarwala, V., Scott, D A., Zhang, F. Nature Protocols November; 8(11):2281-308 (2013-B);
Genome-Scale CRISPR-Cas9 Knockout Screening in Human Cells. Shalem, O., Sanjana, N E., Hartenian, E., Shi, X., Scott, D A., Mikkelson, T., Heckl, D., Ebert, B L., Root, D E., Doench, J G., Zhang, F. Science December 12. (2013);
Crystal structure of cas9 in complex with guide RNA and target DNA. Nishimasu, H., Ran, F A., Hsu, P D., Konermann, S., Shehata, S I., Dohmae, N., Ishitani, R., Zhang, F., Nureki, O. Cell February 27, 156(5):935-49 (2014);
Genome-wide binding of the CRISPR endonuclease Cas9 in mammalian cells. Wu X., Scott D A., Kriz A J., Chiu A C., Hsu P D., Dadon D B., Cheng A W., Trevino A E., Konermann S., Chen S., Jaenisch R., Zhang F., Sharp P A. Nat Biotechnol. April 20. doi: 10.1038/nbt.2889 (2014);
CRISPR-Cas9 Knockin Mice for Genome Editing and Cancer Modeling. Platt R J, Chen S, Zhou Y, Yim M J, Swiech L, Kempton H R, Dahlman J E, Parnas O, Eisenhaure T M, Jovanovic M, Graham D B, Jhunjhunwala S, Heidenreich M, Xavier R J, Langer R, Anderson D G, Hacohen N, Regev A, Feng G, Sharp P A, Zhang F. Cell 159(2): 440-455 DOI: 10.1016/j.cell.2014.09.014(2014);
Development and Applications of CRISPR-Cas9 for Genome Engineering, Hsu P D, Lander E S, Zhang F., Cell. June 5; 157(6):1262-78 (2014).
Genetic screens in human cells using the CRISPR-Cas9 system, Wang T, Wei J J, Sabatini D M, Lander E S., Science. January 3; 343(6166): 80-84. doi:10.1126/science.1246981 (2014);
Rational design of highly active sgRNAs for CRISPR-Cas9-mediated gene inactivation, Doench J G, Hartenian E, Graham D B, Tothova Z, Hegde M, Smith I, Sullender M, Ebert B L, Xavier R J, Root D E., (published online 3 Sep. 2014) Nat Biotechnol. December; 32(12):1262-7 (2014);
In vivo interrogation of gene function in the mammalian brain using CRISPR-Cas9, Swiech L, Heidenreich M, Banerjee A, Habib N, Li Y, Trombetta J, Sur M, Zhang F., (published online 19 Oct. 2014) Nat Biotechnol. January; 33(1):102-6 (2015);
Genome-scale transcriptional activation by an engineered CRISPR-Cas9 complex, Konermann S, Brigham M D, Trevino A E, Joung J, Abudayyeh O O, Barcena C, Hsu P D, Habib N, Gootenberg J S, Nishimasu H, Nureki O, Zhang F., Nature. January 29; 517(7536):583-8 (2015).
A split-Cas9 architecture for inducible genome editing and transcription modulation, Zetsche B, Volz S E, Zhang F., (published online 2 Feb. 2015) Nat Biotechnol. February; 33(2):139-42 (2015);
Genome-wide CRISPR Screen in a Mouse Model of Tumor Growth and Metastasis, Chen S, Sanjana N E, Zheng K, Shalem O, Lee K, Shi X, Scott D A, Song J, Pan J Q, Weissleder R, Lee H, Zhang F, Sharp P A. Cell 160, 1246-1260, Mar. 12, 2015 (multiplex screen in mouse), and
In vivo genome editing using Staphylococcus aureus Cas9, Ran F A, Cong L, Yan W X, Scott D A, Gootenberg J S, Kriz A J, Zetsche B, Shalem O, Wu X, Makarova K S, Koonin E V, Sharp P A, Zhang F., (published online 1 Apr. 2015), Nature. April 9; 520(7546):186-91 (2015).
Shalem et al., “High-throughput functional genomics using CRISPR-Cas9,” Nature Reviews Genetics 16, 299-311 (May 2015).
Xu et al., “Sequence determinants of improved CRISPR sgRNA design,” Genome Research 25, 1147-1157 (August 2015).
Parnas et al., “A Genome-wide CRISPR Screen in Primary Immune Cells to Dissect Regulatory Networks,” Cell 162, 675-686 (Jul. 30, 2015).
Ramanan et al., CRISPR-Cas9 cleavage of viral DNA efficiently suppresses hepatitis B virus,” Scientific Reports 5:10833. doi: 10.1038/srep10833 (Jun. 2, 2015)
Nishimasu et al., Crystal Structure of Staphylococcus aureus Cas9,” Cell 162, 1113-1126 (Aug. 27, 2015)
BCL11A enhancer dissection by Cas9-mediated in situ saturating mutagenesis, Canver et al., Nature 527(7577):192-7 (Nov. 12, 2015) doi: 10.1038/nature15521. Epub 2015 Sep. 16.
Cpf1 Is a Single RNA-Guided Endonuclease of a Class 2 CRISPR-Cas System, Zetsche et al., Cell 163, 759-71 (Sep. 25, 2015).
Discovery and Functional Characterization of Diverse Class 2 CRISPR-Cas Systems, Shmakov et al., Molecular Cell, 60(3), 385-397 doi: 10.1016/j.molcel.2015.10.008 Epub Oct. 22, 2015.
Rationally engineered Cas9 nucleases with improved specificity, Slaymaker et al., Science 2016 Jan. 1 351(6268): 84-88 doi: 10.1126/science.aad5227. Epub 2015 Dec. 1.
Gao et al, “Engineered Cpf1 Enzymes with Altered PAM Specificities,” bioRxiv 091611; doi: http://dx.doi.org/10.1101/091611 (Dec. 4, 2016).
Cox et al., “RNA editing with CRISPR-Cas13,” Science. 2017 Nov. 24; 358(6366):1019-1027. doi: 10.1126/science.aaq0180. Epub 2017 Oct. 25.

each of which is incorporated herein by reference, may be considered in the practice of the instant invention, and discussed briefly below:
Cong et al. engineered type II CRISPR-Cas systems for use in eukaryotic cells based on both Streptococcus thermophilus Cas9 and also Streptococcus pyogenes Cas9 and demonstrated that Cas9 nucleases can be directed by short RNAs to induce precise cleavage of DNA in human and mouse cells. Their study further showed that Cas9 as converted into a nicking enzyme can be used to facilitate homology-directed repair in eukaryotic cells with minimal mutagenic activity. Additionally, their study demonstrated that multiple guide sequences can be encoded into a single CRISPR array to enable simultaneous editing of several at endogenous genomic loci sites within the mammalian genome, demonstrating easy programmability and wide applicability of the RNA-guided nuclease technology. This ability to use RNA to program sequence specific DNA cleavage in cells defined a new class of genome engineering tools. These studies further showed that other CRISPR loci are likely to be transplantable into mammalian cells and can also mediate mammalian genome cleavage. Importantly, it can be envisaged that several aspects of the CRISPR-Cas system can be further improved to increase its efficiency and versatility.
Jiang et al. used the clustered, regularly interspaced, short palindromic repeats (CRISPR)-associated Cas9 endonuclease complexed with dual-RNAs to introduce precise mutations in the genomes of Streptococcuspneumoniae and Escherichia coli. The approach relied on dual-RNA:Cas9-directed cleavage at the targeted genomic site to kill unmutated cells and circumvents the need for selectable markers or counter-selection systems. The study reported reprogramming dual-RNA:Cas9 specificity by changing the sequence of short CRISPR RNA (crRNA) to make single- and multinucleotide changes carried on editing templates. The study showed that simultaneous use of two crRNAs enabled multiplex mutagenesis. Furthermore, when the approach was used in combination with recombineering, in S. pneumoniae, nearly 100% of cells that were recovered using the described approach contained the desired mutation, and in E. coli, 65% that were recovered contained the mutation.
Wang et al. (2013) used the CRISPR-Cas system for the one-step generation of mice carrying mutations in multiple genes which were traditionally generated in multiple steps by sequential recombination in embryonic stem cells and/or time-consuming intercrossing of mice with a single mutation. The CRISPR-Cas system will greatly accelerate the in vivo study of functionally redundant genes and of epistatic gene interactions.
Konermann et al. (2013) addressed the need in the art for versatile and robust technologies that enable optical and chemical modulation of DNA-binding domains based CRISPR Cas9 enzyme and also Transcriptional Activator Like Effectors
Ran et al. (2013-A) described an approach that combined a Cas9 nickase mutant with paired guide RNAs to introduce targeted double-strand breaks. This addresses the issue of the Cas9 nuclease from the microbial CRISPR-Cas system being targeted to specific genomic loci by a guide sequence, which can tolerate certain mismatches to the DNA target and thereby promote undesired off-target mutagenesis. Because individual nicks in the genome are repaired with high fidelity, simultaneous nicking via appropriately offset guide RNAs is required for double-stranded breaks and extends the number of specifically recognized bases for target cleavage. The authors demonstrated that using paired nicking can reduce off-target activity by 50- to 1,500-fold in cell lines and to facilitate gene knockout in mouse zygotes without sacrificing on-target cleavage efficiency. This versatile strategy enables a wide variety of genome editing applications that require high specificity.
Hsu et al. (2013) characterized SpCas9 targeting specificity in human cells to inform the selection of target sites and avoid off-target effects. The study evaluated >700 guide RNA variants and SpCas9-induced indel mutation levels at >100 predicted genomic off-target loci in 293T and 293FT cells. The authors that SpCas9 tolerates mismatches between guide RNA and target DNA at different positions in a sequence-dependent manner, sensitive to the number, position and distribution of mismatches. The authors further showed that SpCas9-mediated cleavage is unaffected by DNA methylation and that the dosage of SpCas9 and guide RNA can be titrated to minimize off-target modification. Additionally, to facilitate mammalian genome engineering applications, the authors reported providing a web-based software tool to guide the selection and validation of target sequences as well as off-target analyses.
Ran et al. (2013-B) described a set of tools for Cas9-mediated genome editing via non-homologous end joining (NHEJ) or homology-directed repair (HDR) in mammalian cells, as well as generation of modified cell lines for downstream functional studies. To minimize off-target cleavage, the authors further described a double-nicking strategy using the Cas9 nickase mutant with paired guide RNAs. The protocol provided by the authors experimentally derived guidelines for the selection of target sites, evaluation of cleavage efficiency and analysis of off-target activity. The studies showed that beginning with target design, gene modifications can be achieved within as little as 1-2 weeks, and modified clonal cell lines can be derived within 2-3 weeks.
Shalem et al. described a new way to interrogate gene function on a genome-wide scale. Their studies showed that delivery of a genome-scale CRISPR-Cas9 knockout (GeCKO) library targeted 18,080 genes with 64,751 unique guide sequences enabled both negative and positive selection screening in human cells. First, the authors showed use of the GeCKO library to identify genes essential for cell viability in cancer and pluripotent stem cells. Next, in a melanoma model, the authors screened for genes whose loss is involved in resistance to vemurafenib, a therapeutic that inhibits mutant protein kinase BRAF. Their studies showed that the highest-ranking candidates included previously validated genes NF1 and MED12 as well as novel hits NF2, CUL3, TADA2B, and TADA1. The authors observed a high level of consistency between independent guide RNAs targeting the same gene and a high rate of hit confirmation, and thus demonstrated the promise of genome-scale screening with Cas9.
Nishimasu et al. reported the crystal structure of Streptococcus pyogenes Cas9 in complex with sgRNA and its target DNA at 2.5 A° resolution. The structure revealed a bilobed architecture composed of target recognition and nuclease lobes, accommodating the sgRNA:DNA heteroduplex in a positively charged groove at their interface. Whereas the recognition lobe is essential for binding sgRNA and DNA, the nuclease lobe contains the HNH and RuvC nuclease domains, which are properly positioned for cleavage of the complementary and non-complementary strands of the target DNA, respectively. The nuclease lobe also contains a carboxyl-terminal domain responsible for the interaction with the protospacer adjacent motif (PAM). This high-resolution structure and accompanying functional analyses have revealed the molecular mechanism of RNA-guided DNA targeting by Cas9, thus paving the way for the rational design of new, versatile genome-editing technologies.
Wu et al. mapped genome-wide binding sites of a catalytically inactive Cas9 (dCas9) from Streptococcus pyogenes loaded with single guide RNAs (sgRNAs) in mouse embryonic stem cells (mESCs). The authors showed that each of the four sgRNAs tested targets dCas9 to between tens and thousands of genomic sites, frequently characterized by a 5-nucleotide seed region in the sgRNA and an NGG protospacer adjacent motif (PAM). Chromatin inaccessibility decreases dCas9 binding to other sites with matching seed sequences; thus 70% of off-target sites are associated with genes. The authors showed that targeted sequencing of 295 dCas9 binding sites in mESCs transfected with catalytically active Cas9 identified only one site mutated above background levels. The authors proposed a two-state model for Cas9 binding and cleavage, in which a seed match triggers binding but extensive pairing with target DNA is required for cleavage.
Platt et al. established a Cre-dependent Cas9 knockin mouse. The authors demonstrated in vivo as well as ex vivo genome editing using adeno-associated virus (AAV)-, lentivirus-, or particle-mediated delivery of guide RNA in neurons, immune cells, and endothelial cells.
Hsu et al. (2014) is a review article that discusses generally CRISPR-Cas9 history from yogurt to genome editing, including genetic screening of cells.
Wang et al. (2014) relates to a pooled, loss-of-function genetic screening approach suitable for both positive and negative selection that uses a genome-scale lentiviral single guide RNA (sgRNA) library.
Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.
Swiech et al. demonstrate that AAV-mediated SpCas9 genome editing can enable reverse genetic studies of gene function in the brain.
Konermann et al. (2015) discusses the ability to attach multiple effector domains, e.g., transcriptional activator, functional and epigenomic regulators at appropriate positions on the guide such as stem or tetraloop with and without linkers.
Zetsche et al. demonstrates that the Cas9 enzyme can be split into two and hence the assembly of Cas9 for activation can be controlled.
Chen et al. relates to multiplex screening by demonstrating that a genome-wide in vivo CRISPR-Cas9 screen in mice reveals genes regulating lung metastasis.
Ran et al. (2015) relates to SaCas9 and its ability to edit genomes and demonstrates that one cannot extrapolate from biochemical assays.
Shalem et al. (2015) described ways in which catalytically inactive Cas9 (dCas9) fusions are used to synthetically repress (CRISPRi) or activate (CRISPRa) expression, showing. advances using Cas9 for genome-scale screens, including arrayed and pooled screens, knockout approaches that inactivate genomic loci and strategies that modulate transcriptional activity.
Xu et al. (2015) assessed the DNA sequence features that contribute to single guide RNA (sgRNA) efficiency in CRISPR-based screens. The authors explored efficiency of CRISPR-Cas9 knockout and nucleotide preference at the cleavage site. The authors also found that the sequence preference for CRISPRi/a is substantially different from that for CRISPR-Cas9 knockout.
Parnas et al. (2015) introduced genome-wide pooled CRISPR-Cas9 libraries into dendritic cells (DCs) to identify genes that control the induction of tumor necrosis factor (Tnf) by bacterial lipopolysaccharide (LPS). Known regulators of Tlr4 signaling and previously unknown candidates were identified and classified into three functional modules with distinct effects on the canonical responses to LPS.
Ramanan et al (2015) demonstrated cleavage of viral episomal DNA (cccDNA) in infected cells. The HBV genome exists in the nuclei of infected hepatocytes as a 3.2 kb double-stranded episomal DNA species called covalently closed circular DNA (cccDNA), which is a key component in the HBV life cycle whose replication is not inhibited by current therapies. The authors showed that sgRNAs specifically targeting highly conserved regions of HBV robustly suppresses viral replication and depleted cccDNA.
Nishimasu et al. (2015) reported the crystal structures of SaCas9 in complex with a single guide RNA (sgRNA) and its double-stranded DNA targets, containing the 5′-TTGAAT-3′ PAM and the 5′-TTGGGT-3′ PAM. A structural comparison of SaCas9 with SpCas9 highlighted both structural conservation and divergence, explaining their distinct PAM specificities and orthologous sgRNA recognition.
Canver et al. (2015) demonstrated a CRISPR-Cas9-based functional investigation of non-coding genomic elements. The authors Applicants developed pooled CRISPR-Cas9 guide RNA libraries to perform in situ saturating mutagenesis of the human and mouse BCL11A enhancers which revealed critical features of the enhancers.
Zetsche et al. (2015) reported characterization of Cpf1, a class 2 CRISPR nuclease from Francisella novicida U112 having features distinct from Cas9. Cpf1 is a single RNA-guided endonuclease lacking tracrRNA, utilizes a T-rich protospacer-adjacent motif, and cleaves DNA via a staggered DNA double-stranded break.
Shmakov et al. (2015) reported three distinct Class 2 CRISPR-Cas systems. Two system CRISPR enzymes (C2c1 and C2c3) contain RuvC-like endonuclease domains distantly related to Cpf1. Unlike Cpf1, C2c1 depends on both crRNA and tracrRNA for DNA cleavage. The third enzyme (C2c2) contains two predicted HEPN RNase domains and is tracrRNA independent.
Slaymaker et al (2016) reported the use of structure-guided protein engineering to improve the specificity of Streptococcus pyogenes Cas9 (SpCas9). The authors developed “enhanced specificity” SpCas9 (eSpCas9) variants which maintained robust on-target cleavage with reduced off-target effects.
Cox et al., (2017) reported the use of catalytically inactive Cas13 (dCas13) to direct adenosine-to-inosine deaminase activity by ADAR2 (adenosine deaminase acting on RNA type 2) to transcripts in mammalian cells. The system, referred to as RNA Editing for Programmable A to I Replacement (REPAIR), has no strict sequence constraints and can be used to edit full-length transcripts. The authors further engineered the system to create a high-specificity variant and minimized the system to facilitate viral delivery.

The methods and tools provided herein are may be designed for use with or Cas13, a type II nuclease that does not make use of tracrRNA. Orthologs of Cas13 have been identified in different bacterial species as described herein. Further type II nucleases with similar properties can be identified using methods described in the art (Shmakov et al. 2015, 60:385-397; Abudayeh et al. 2016, Science, 5; 353(6299)). In particular embodiments, such methods for identifying novel CRISPR effector proteins may comprise the steps of selecting sequences from the database encoding a seed which identifies the presence of a CRISPR Cas locus, identifying loci located within 10 kb of the seed comprising Open Reading Frames (ORFs) in the selected sequences, selecting therefrom loci comprising ORFs of which only a single ORF encodes a novel CRISPR effector having greater than 700 amino acids and no more than 90% homology to a known CRISPR effector. In particular embodiments, the seed is a protein that is common to the CRISPR-Cas system, such as Cas1. In further embodiments, the CRISPR array is used as a seed to identify new effector proteins.

Also, “Dimeric CRISPR RNA-guided FokI nucleases for highly specific genome editing”, Shengdar Q. Tsai, Nicolas Wyvekens, Cyd Khayter, Jennifer A. Foden, Vishal Thapar, Deepak Reyon, Mathew J. Goodwin, Martin J. Aryee, J. Keith Joung Nature Biotechnology 32(6): 569-77 (2014), relates to dimeric RNA-guided FokI Nucleases that recognize extended sequences and can edit endogenous genes with high efficiencies in human cells.

With respect to general information on CRISPR/Cas Systems, components thereof, and delivery of such components, including methods, materials, delivery vehicles, vectors, particles, and making and using thereof, including as to amounts and formulations, as well as CRISPR-Cas-expressing eukaryotic cells, CRISPR-Cas expressing eukaryotes, such as a mouse, reference is made to: U.S. Pat. Nos. 8,999,641, 8,993,233, 8,697,359, 8,771,945, 8,795,965, 8,865,406, 8,871,445, 8,889,356, 8,889,418, 8,895,308, 8,906,616, 8,932,814, and 8,945,839; US Patent Publications US 2014-0310830 (U.S. application Ser. No. 14/105,031), US 2014-0287938 A1 (U.S. application Ser. No. 14/213,991), US 2014-0273234 A1 (U.S. application Ser. No. 14/293,674), US2014-0273232 A1 (U.S. application Ser. No. 14/290,575), US 2014-0273231 (U.S. application Ser. No. 14/259,420), US 2014-0256046 A1 (U.S. application Ser. No. 14/226,274), US 2014-0248702 A1 (U.S. application Ser. No. 14/258,458), US 2014-0242700 A1 (U.S. application Ser. No. 14/222,930), US 2014-0242699 A1 (U.S. application Ser. No. 14/183,512), US 2014-0242664 A1 (U.S. application Ser. No. 14/104,990), US 2014-0234972 A1 (U.S. application Ser. No. 14/183,471), US 2014-0227787 A1 (U.S. application Ser. No. 14/256,912), US 2014-0189896 A1 (U.S. application Ser. No. 14/105,035), US 2014-0186958 (U.S. application Ser. No. 14/105,017), US 2014-0186919 A1 (U.S. application Ser. No. 14/104,977), US 2014-0186843 A1 (U.S. application Ser. No. 14/104,900), US 2014-0179770 A1 (U.S. application Ser. No. 14/104,837) and US 2014-0179006 A1 (U.S. application Ser. No. 14/183,486), US 2014-0170753 (U.S. application Ser. No. 14/183,429); US 2015-0184139 (U.S. application Ser. No. 14/324,960); 14/054,414 European Patent Applications EP 2 771 468 (EP13818570.7), EP 2 764 103 (EP13824232.6), and EP 2 784 162 (EP14170383.5); and PCT Patent Publications

WO2014/093661 (PCT/US2013/074743), WO2014/093694 (PCT/US2013/074790),
WO2014/093595 (PCT/US2013/074611), WO2014/093718 (PCT/US2013/074825),
WO2014/093709 (PCT/US2013/074812), WO2014/093622 (PCT/US2013/074667),
WO2014/093635 (PCT/US2013/074691), WO2014/093655 (PCT/US2013/074736),
WO2014/093712 (PCT/US2013/074819), WO2014/093701 (PCT/US2013/074800),
WO2014/018423 (PCT/US2013/051418), WO2014/204723 (PCT/US2014/041790),
WO2014/204724 (PCT/US2014/041800), WO2014/204725 (PCT/US2014/041803),
WO2014/204726 (PCT/US2014/041804), WO2014/204727 (PCT/US2014/041806),
WO2014/204728 (PCT/US2014/041808), WO2014/204729 (PCT/US2014/041809),
WO2015/089351 (PCT/US2014/069897), WO2015/089354 (PCT/US2014/069902),
WO2015/089364 (PCT/US2014/069925), WO2015/089427 (PCT/US2014/070068),
WO2015/089462 (PCT/US2014/070127), WO2015/089419 (PCT/US2014/070057),
WO2015/089465 (PCT/US2014/070135), WO2015/089486 (PCT/US2014/070175),
WO2015/058052 (PCT/US2014/061077), WO2015/070083 (PCT/US2014/064663),
WO2015/089354 (PCT/US2014/069902), WO2015/089351 (PCT/US2014/069897),
WO2015/089364 (PCT/US2014/069925), WO2015/089427 (PCT/US2014/070068),
WO2015/089473 (PCT/US2014/070152), WO2015/089486 (PCT/US2014/070175),
WO2016/049258 (PCT/US2015/051830), WO2016/094867 (PCT/US2015/065385),
WO2016/094872 (PCT/US2015/065393), WO2016/094874 (PCT/US2015/065396),
WO2016/106244 (PCT/US2015/067177).

Mention is also made of U.S. application 62/180,709, 17 Jun. 2015, PROTECTED GUIDE RNAS (PGRNAS); U.S. application 62/091,455, filed, 12 Dec. 2014, PROTECTED GUIDE RNAS (PGRNAS); U.S. application 62/096,708, 24 Dec. 2014, PROTECTED GUIDE RNAS (PGRNAS); U.S. applications 62/091,462, 12 Dec. 2014, 62/096,324, 23 Dec. 2014, 62/180,681, 17 Jun. 2015, and 62/237,496, 5 Oct. 2015, DEAD GUIDES FOR CRISPR TRANSCRIPTION FACTORS; U.S. application 62/091,456, 12 Dec. 2014 and 62/180,692, 17 Jun. 2015, ESCORTED AND FUNCTIONALIZED GUIDES FOR CRISPR-CAS SYSTEMS; U.S. application 62/091,461, 12 Dec. 2014, DELIVERY, USE AND THERAPEUTIC APPLICATIONS OF THE CRISPR-CAS SYSTEMS AND COMPOSITIONS FOR GENOME EDITING AS TO HEMATOPOETIC STEM CELLS (HSCs); U.S. application 62/094,903, 19 Dec. 2014, UNBIASED IDENTIFICATION OF DOUBLE-STRAND BREAKS AND GENOMIC REARRANGEMENT BY GENOME-WISE INSERT CAPTURE SEQUENCING; U.S. application 62/096,761, 24 Dec. 2014, ENGINEERING OF SYSTEMS, METHODS AND OPTIMIZED ENZYME AND GUIDE SCAFFOLDS FOR SEQUENCE MANIPULATION; U.S. application 62/098,059, 30 Dec. 2014, 62/181,641, 18 Jun. 2015, and 62/181,667, 18 Jun. 2015, RNA-TARGETING SYSTEM; U.S. application 62/096,656, 24 Dec. 2014 and 62/181,151, 17 Jun. 2015, CRISPR HAVING OR ASSOCIATED WITH DESTABILIZATION DOMAINS; U.S. application 62/096,697, 24 Dec. 2014, CRISPR HAVING OR ASSOCIATED WITH AAV; U.S. application 62/098,158, 30 Dec. 2014, ENGINEERED CRISPR COMPLEX INSERTIONAL TARGETING SYSTEMS; U.S. application 62/151,052, 22 Apr. 2015, CELLULAR TARGETING FOR EXTRACELLULAR EXOSOMAL REPORTING; U.S. application 62/054,490, 24 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS; U.S. application 61/939,154, 12 Feb. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/055,484, 25 Sep. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/087,537, 4 Dec. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/054,651, 24 Sep. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/067,886, 23 Oct. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. applications 62/054,675, 24 Sep. 2014 and 62/181,002, 17 Jun. 2015, DELIVERY, USE AND THERAPEUTIC APPLICATIONS OF THE CRISPR-CAS SYSTEMS AND COMPOSITIONS IN NEURONAL CELLS/TISSUES; U.S. application 62/054,528, 24 Sep. 2014, DELIVERY, USE AND THERAPEUTIC APPLICATIONS OF THE CRISPR-CAS SYSTEMS AND COMPOSITIONS IN IMMUNE DISEASES OR DISORDERS; U.S. application 62/055,454, 25 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING CELL PENETRATION PEPTIDES (CPP); U.S. application 62/055,460, 25 Sep. 2014, MULTIFUNCTIONAL-CRISPR COMPLEXES AND/OR OPTIMIZED ENZYME LINKED FUNCTIONAL-CRISPR COMPLEXES; U.S. application 62/087,475, 4 Dec. 2014 and 62/181,690, 18 Jun. 2015, FUNCTIONAL SCREENING WITH OPTIMIZED FUNCTIONAL CRISPR-CAS SYSTEMS; U.S. application 62/055,487, 25 Sep. 2014, FUNCTIONAL SCREENING WITH OPTIMIZED FUNCTIONAL CRISPR-CAS SYSTEMS; U.S. application 62/087,546, 4 Dec. 2014 and 62/181,687, 18 Jun. 2015, MULTIFUNCTIONAL CRISPR COMPLEXES AND/OR OPTIMIZED ENZYME LINKED FUNCTIONAL-CRISPR COMPLEXES; and U.S. application 62/098,285, 30 Dec. 2014, CRISPR MEDIATED IN VIVO MODELING AND GENETIC SCREENING OF TUMOR GROWTH AND METASTASIS.

Mention is made of U.S. applications 62/181,659, 18 Jun. 2015 and 62/207,318, 19 Aug. 2015, ENGINEERING AND OPTIMIZATION OF SYSTEMS, METHODS, ENZYME AND GUIDE SCAFFOLDS OF CAS9 ORTHOLOGS AND VARIANTS FOR SEQUENCE MANIPULATION. Mention is made of U.S. applications 62/181,663, 18 Jun. 2015 and 62/245,264, 22 Oct. 2015, NOVEL CRISPR ENZYMES AND SYSTEMS, U.S. applications 62/181,675, 18 Jun. 2015, 62/285,349, 22 Oct. 2015, 62/296,522, 17 Feb. 2016, and 62/320,231, 8 Apr. 2016, NOVEL CRISPR ENZYMES AND SYSTEMS, U.S. application 62/232,067, 24 Sep. 2015, U.S. application Ser. No. 14/975,085, 18 Dec. 2015, European application No. 16150428.7, U.S. application 62/205,733, 16 Aug. 2015, U.S. application 62/201,542, 5 Aug. 2015, U.S. application 62/193,507, 16 Jul. 2015, and U.S. application 62/181,739, 18 Jun. 2015, each entitled NOVEL CRISPR ENZYMES AND SYSTEMS and of U.S. application 62/245,270, 22 Oct. 2015, NOVEL CRISPR ENZYMES AND SYSTEMS. Mention is also made of U.S. application 61/939,256, 12 Feb. 2014, and WO 2015/089473 (PCT/US2014/070152), 12 Dec. 2014, each entitled ENGINEERING OF SYSTEMS, METHODS AND OPTIMIZED GUIDE COMPOSITIONS WITH NEW ARCHITECTURES FOR SEQUENCE MANIPULATION. Mention is also made of PCT/US2015/045504, 15 Aug. 2015, U.S. application 62/180,699, 17 Jun. 2015, and U.S. application 62/038,358, 17 Aug. 2014, each entitled GENOME EDITING USING CAS9 NICKASES.

Each of these patents, patent publications, and applications, and all documents cited therein or during their prosecution (“appln cited documents”) and all documents cited or referenced in the appln cited documents, together with any instructions, descriptions, product specifications, and product sheets for any products mentioned therein or in any document therein and incorporated by reference herein, are hereby incorporated herein by reference, and may be employed in the practice of the invention. All documents (e.g., these patents, patent publications and applications and the appln cited documents) are incorporated herein by reference to the same extent as if each individual document was specifically and individually indicated to be incorporated by reference.

In particular embodiments, pre-complexed guide RNA and CRISPR effector protein, (optionally, adenosine deaminase fused to a CRISPR protein or an adaptor) are delivered as a ribonucleoprotein (RNP). RNPs have the advantage that they lead to rapid editing effects even more so than the RNA method because this process avoids the need for transcription. An important advantage is that both RNP delivery is transient, reducing off-target effects and toxicity issues. Efficient genome editing in different cell types has been observed by Kim et al. (2014, Genome Res. 24(6):1012-9), Paix et al. (2015, Genetics 204(1):47-54), Chu et al. (2016, BMC Biotechnol. 16:4), and Wang et al. (2013, Cell. 9; 153(4):910-8).

In particular embodiments, the ribonucleoprotein is delivered by way of a polypeptide-based shuttle agent as described in WO2016161516. WO2016161516 describes efficient transduction of polypeptide cargos using synthetic peptides comprising an endosome leakage domain (ELD) operably linked to a cell penetrating domain (CPD), to a histidine-rich domain and a CPD. Similarly these polypeptides can be used for the delivery of CRISPR-effector based RNPs in eukaryotic cells.

### Tale Systems

As disclosed herein editing can be made by way of the transcription activator-like effector nucleases (TALENs) system. Transcription activator-like effectors (TALEs) can be engineered to bind practically any desired DNA sequence. Exemplary methods of genome editing using the TALEN system can be found for example in Cermak T. Doyle E L. Christian M. Wang L. Zhang Y. Schmidt C, et al. Efficient design and assembly of custom TALEN and other TAL effector-based constructs for DNA targeting. Nucleic Acids Res. 2011; 39:e82; Zhang F. Cong L. Lodato S. Kosuri S. Church G M. Arlotta P Efficient construction of sequence-specific TAL effectors for modulating mammalian transcription. Nat Biotechnol. 2011; 29:149-153 and U.S. Pat. Nos. 8,450,471, 8,440,431 and 8,440,432, all of which are specifically incorporated by reference.

In advantageous embodiments of the invention, the methods provided herein use isolated, non-naturally occurring, recombinant or engineered DNA binding proteins that comprise TALE monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, or “TALE monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X1-11-(X12X13)-X14-33 or 34 or 35, where the subscript indicates the amino acid position and X represents any amino acid. X12X13 indicate the RVDs. In some polypeptide monomers, the variable amino acid at position 13 is missing or absent and in such polypeptide monomers, the RVD consists of a single amino acid. In such cases the RVD may be alternatively represented as X*, where X represents X12 and (*) indicates that X13 is absent. The DNA binding domain comprises several repeats of TALE monomers and this may be represented as (X1-11-(X12X13)-X14-33 or 34 or 35)z, where in an advantageous embodiment, z is at least 5 to 40. In a further advantageous embodiment, z is at least 10 to 26.

The TALE monomers have a nucleotide binding affinity that is determined by the identity of the amino acids in its RVD. For example, polypeptide monomers with an RVD of NI preferentially bind to adenine (A), polypeptide monomers with an RVD of NG preferentially bind to thymine (T), polypeptide monomers with an RVD of HD preferentially bind to cytosine (C) and polypeptide monomers with an RVD of NN preferentially bind to both adenine (A) and guanine (G). In yet another embodiment of the invention, polypeptide monomers with an RVD of IG preferentially bind to T. Thus, the number and order of the polypeptide monomer repeats in the nucleic acid binding domain of a TALE determines its nucleic acid target specificity. In still further embodiments of the invention, polypeptide monomers with an RVD of NS recognize all four base pairs and may bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011), each of which is incorporated by reference in its entirety.

The TALE polypeptides used in methods of the invention are isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In a preferred embodiment of the invention, polypeptide monomers having RVDs RN, NN, NK, SN, NH, KN, HN, NQ, HH, RG, KH, RH and SS preferentially bind to guanine. In a much more advantageous embodiment of the invention, polypeptide monomers having RVDs RN, NK, NQ, HH, KH, RH, SS and SN preferentially bind to guanine and thereby allow the generation of TALE polypeptides with high binding specificity for guanine containing target nucleic acid sequences. In an even more advantageous embodiment of the invention, polypeptide monomers having RVDs HH, KH, NH, NK, NQ, RH, RN and SS preferentially bind to guanine and thereby allow the generation of TALE polypeptides with high binding specificity for guanine containing target nucleic acid sequences. In a further advantageous embodiment, the RVDs that have high binding specificity for guanine are RN, NH RH and KH. Furthermore, polypeptide monomers having an RVD of NV preferentially bind to adenine and guanine. In more preferred embodiments of the invention, polypeptide monomers having RVDs of H*, HA, KA, N*, NA, NC, NS, RA, and S* bind to adenine, guanine, cytosine and thymine with comparable affinity.

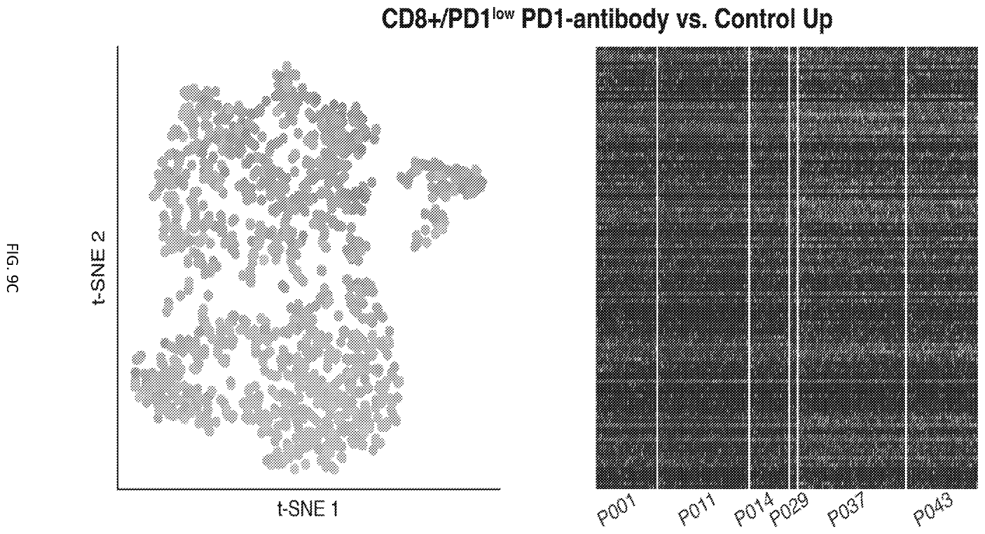

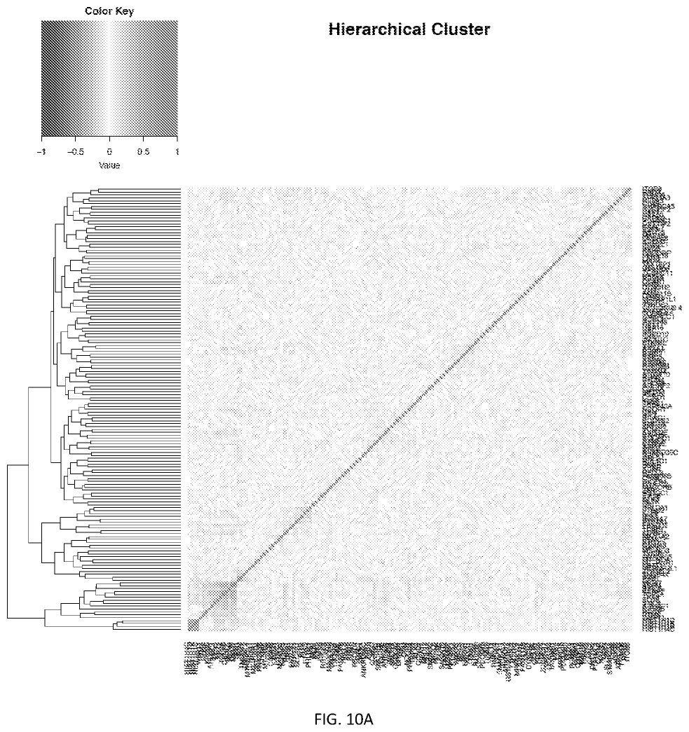

The predetermined N-terminal to C-terminal order of the one or more polypeptide monomers of the nucleic acid or DNA binding domain determines the corresponding predetermined target nucleic acid sequence to which the TALE polypeptides will bind. As used herein the polypeptide monomers and at least one or more half polypeptide monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases this region may be referred to as repeat 0. In animal genomes, TALE binding sites do not necessarily have to begin with a thymine (T) and TALE polypeptides may target DNA sequences that begin with T, A, G or C. The tandem repeat of TALE monomers always ends with a half-length repeat or a stretch of sequence that may share identity with only the first 20 amino acids of a repetitive full length TALE monomer and this half repeat may be referred to as a half-monomer (FIG. 8), which is included in the term “TALE monomer”. Therefore, it follows that the length of the nucleic acid or DNA being targeted is equal to the number of full polypeptide monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ. ID. No. 4)
M D P I R S R T P S P A R E L L S G P Q P D G V Q
P T A D R G V S P P A G G P L D G L P A R R T M S
R T R L P S P P A P S P A F S A D S F S D L L R Q
F D P S L F N T S L F D S L P P F G A H H T E A A
T G E W D E V Q S G L R A A D A P P P T M R V A V
T A A R P P R A K P A P R R R A A Q P S D A S P A
A Q V D L R T L G Y S Q Q Q Q E K I K P K V R S T
V A Q H H E A L V G H G F T H A H I V A L S Q H P
A A L G T V A V K Y Q D M I A A L P E A T H E A I
V G V G K Q W S G A R A L E A L L T V A G E L R G
P P L Q L D T G Q L L K I A K R G G V T A V E A V
H A W R N A L T G A P L N

An exemplary amino acid sequence of a C-terminal capping region is:

(SEQ. I.D. No. 5)
R P A L E S I V A Q L S R P D P A L A A L T N D H
L V A L A C L G G R P A L D A V K K G L P H A P A
L I K R T N R R I P E R T S H R V A D H A Q V V R
V L G F F Q C H S H P A Q A F D D A M T Q F G M S
R H G L L Q L F R R V G V T E L E A R S G T L P P
A S Q R W D R I L Q A S G M K R A K P S P T S T Q
T P D Q A S L H A F A D S L E R D L D A P S P M H
E G D Q T R A S

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full length capping region, while fragments that include the C-terminal 20 amino acids retain greater than 50% of the efficacy of the full length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies may be generated by any of a number of computer programs known in the art, which include but are not limited to BLAST or FASTA. Suitable computer program for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In advantageous embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Kruppel-associated box (KRAB) or fragments of the KRAB domain. In some embodiments the effector domain is an enhancer of transcription (i.e. an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination the activities described herein.

### ZN-Finger Nucleases

Other preferred tools for genome editing for use in the context of this invention include zinc finger systems and TALE systems. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### Meganucleases

As disclosed herein editing can be made by way of meganucleases, which are endodeoxyribonucleases characterized by a large recognition site (double-stranded DNA sequences of 12 to 40 base pairs). Exemplary method for using meganucleases can be found in U.S. Pat. Nos. 8,163,514; 8,133,697; 8,021,867; 8,119,361; 8,119,381; 8,124,369; and 8,129,134, which are specifically incorporated by reference.

### Diseases

It will be understood by the skilled person that treating as referred to herein encompasses enhancing treatment, or improving treatment efficacy. Treatment may include inhibition of tumor regression as well as inhibition of tumor growth, metastasis or tumor cell proliferation, or inhibition or reduction of otherwise deleterious effects associated with the tumor.

Efficaciousness of treatment is determined in association with any known method for diagnosing or treating the particular disease. The invention comprehends a treatment method comprising any one of the methods or uses herein discussed.

The phrase “therapeutically effective amount” as used herein refers to a sufficient amount of a drug, agent, or compound to provide a desired therapeutic effect.

As used herein “patient” refers to any human being receiving or who may receive medical treatment and is used interchangeably herein with the term “subject”.

Therapy or treatment according to the invention may be performed alone or in conjunction with another therapy, and may be provided at home, the doctor's office, a clinic, a hospital's outpatient department, or a hospital. Treatment generally begins at a hospital so that the doctor can observe the therapy's effects closely and make any adjustments that are needed. The duration of the therapy depends on the age and condition of the patient, the stage of the cancer, and how the patient responds to the treatment.

The disclosure also provides methods for reducing resistance to immunotherapy and treating disease. Not being bound by a theory, cancer cells have many strategies of avoiding the immune system and by reducing the signature of the present invention cancer cells may be unmasked to the immune system. Not being bound by a theory, reducing a gene signature of the present invention may be used to treat a subject who has not been administered an immunotherapy, such that the subject's tumor becomes unmasked to their natural or unamplified immune system. In other embodiments, the cancer is resistant to therapies targeting the adaptive immune system (see e.g., Rooney et al., Molecular and genetic properties of tumors associated with local immune cytolytic activity, Cell. 2015 Jan. 15; 160(1-2): 48-61). In one embodiment, modulation of one or more of the signature genes are used for reducing an immunotherapy resistant signature for the treatment of a subpopulation of tumor cells that are linked to resistance to targeted therapies and progressive tumor growth.

In general, the immune system is involved with controlling all cancers and the present application is applicable to treatment of all cancers. Not being bound by a theory, the signature of the present invention is applicable to all cancers and may be used for treatment, as well as for determining a prognosis and stratifying patients. The cancer may include, without limitation, liquid tumors such as leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease, non-Hodgkin's disease), Waldenstrom's macroglobulinemia, heavy chain disease, or multiple myeloma.

The cancer may include, without limitation, solid tumors such as sarcomas and carcinomas. Examples of solid tumors include, but are not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, epithelial carcinoma, bronchogenic carcinoma, hepatoma, colorectal cancer (e.g., colon cancer, rectal cancer), anal cancer, pancreatic cancer (e.g., pancreatic adenocarcinoma, islet cell carcinoma, neuroendocrine tumors), breast cancer (e.g., ductal carcinoma, lobular carcinoma, inflammatory breast cancer, clear cell carcinoma, mucinous carcinoma), ovarian carcinoma (e.g., ovarian epithelial carcinoma or surface epithelial-stromal tumour including serous tumour, endometrioid tumor and mucinous cystadenocarcinoma, sex-cord-stromal tumor), prostate cancer, liver and bile duct carcinoma (e.g., hepatocelluar carcinoma, cholangiocarcinoma, hemangioma), choriocarcinoma, seminoma, embryonal carcinoma, kidney cancer (e.g., renal cell carcinoma, clear cell carcinoma, Wilm's tumor, nephroblastoma), cervical cancer, uterine cancer (e.g., endometrial adenocarcinoma, uterine papillary serous carcinoma, uterine clear-cell carcinoma, uterine sarcomas and leiomyosarcomas, mixed mullerian tumors), testicular cancer, germ cell tumor, lung cancer (e.g., lung adenocarcinoma, squamous cell carcinoma, large cell carcinoma, bronchioloalveolar carcinoma, non-small-cell carcinoma, small cell carcinoma, mesothelioma), bladder carcinoma, signet ring cell carcinoma, cancer of the head and neck (e.g., squamous cell carcinomas), esophageal carcinoma (e.g., esophageal adenocarcinoma), tumors of the brain (e.g., glioma, glioblastoma, medullablastoma, astrocytoma, medulloblastoma, craniopharyngioma, ependymoma, pinealoma, hemangioblastoma, acoustic neuroma, oligodenroglioma, schwannoma, meningioma), neuroblastoma, retinoblastoma, neuroendocrine tumor, melanoma, cancer of the stomach (e.g., stomach adenocarcinoma, gastrointestinal stromal tumor), or carcinoids. Lymphoproliferative disorders are also considered to be proliferative diseases.

Administration It will be appreciated that administration of therapeutic entities in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000), Charman W N “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

The medicaments of the invention are prepared in a manner known to those skilled in the art, for example, by means of conventional dissolving, lyophilizing, mixing, granulating or confectioning processes. Methods well known in the art for making formulations are found, for example, in Remington: The Science and Practice of Pharmacy, 20th ed., ed. A. R. Gennaro, 2000, Lippincott Williams & Wilkins, Philadelphia, and Encyclopedia of Pharmaceutical Technology, eds. J. Swarbrick and J. C. Boylan, 1988-1999, Marcel Dekker, New York.

Administration of medicaments of the invention may be by any suitable means that results in a compound concentration that is effective for treating or inhibiting (e.g., by delaying) the development of a disease. The compound is admixed with a suitable carrier substance, e.g., a pharmaceutically acceptable excipient that preserves the therapeutic properties of the compound with which it is administered. One exemplary pharmaceutically acceptable excipient is physiological saline. The suitable carrier substance is generally present in an amount of 1-95% by weight of the total weight of the medicament. The medicament may be provided in a dosage form that is suitable for administration. Thus, the medicament may be in form of, e.g., tablets, capsules, pills, powders, granulates, suspensions, emulsions, solutions, gels including hydrogels, pastes, ointments, creams, plasters, drenches, delivery devices, injectables, implants, sprays, or aerosols.

The agents disclosed herein (e.g., antibodies) may be used in a pharmaceutical composition when combined with a pharmaceutically acceptable carrier. Such compositions comprise a therapeutically-effective amount of the agent and a pharmaceutically acceptable carrier. Such a composition may also further comprise (in addition to an agent and a carrier) diluents, fillers, salts, buffers, stabilizers, solubilizers, and other materials well known in the art. Compositions comprising the agent can be administered in the form of salts provided the salts are pharmaceutically acceptable. Salts may be prepared using standard procedures known to those skilled in the art of synthetic organic chemistry.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. The term “pharmaceutically acceptable salt” further includes all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and the like which can be used as a dosage form for modifying the solubility or hydrolysis characteristics or can be used in sustained release or prodrug formulations. It will be understood that, as used herein, references to specific agents (e.g., neuromedin U receptor agonists or antagonists), also include the pharmaceutically acceptable salts thereof.

Methods of administrating the pharmacological compositions, including agonists, antagonists, antibodies or fragments thereof, to an individual include, but are not limited to, intradermal, intrathecal, intramuscular, intraperitoneal, intravenous, subcutaneous, intranasal, epidural, by inhalation, and oral routes. The compositions can be administered by any convenient route, for example by infusion or bolus injection, by absorption through epithelial or mucocutaneous linings (for example, oral mucosa, rectal and intestinal mucosa, and the like), ocular, and the like and can be administered together with other biologically-active agents. Administration can be systemic or local. In addition, it may be advantageous to administer the composition into the central nervous system by any suitable route, including intraventricular and intrathecal injection. Pulmonary administration may also be employed by use of an inhaler or nebulizer, and formulation with an aerosolizing agent. It may also be desirable to administer the agent locally to the area in need of treatment; this may be achieved by, for example, and not by way of limitation, local infusion during surgery, topical application, by injection, by means of a catheter, by means of a suppository, or by means of an implant.

Various delivery systems are known and can be used to administer the pharmacological compositions including, but not limited to, encapsulation in liposomes, microparticles, microcapsules; minicells; polymers; capsules; tablets; and the like. In one embodiment, the agent may be delivered in a vesicle, in particular a liposome. In a liposome, the agent is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,837,028 and 4,737,323. In yet another embodiment, the pharmacological compositions can be delivered in a controlled release system including, but not limited to: a delivery pump (See, for example, Saudek, et al., New Engl. J. Med. 321: 574 (1989) and a semi-permeable polymeric material (See, for example, Howard, et al., J. Neurosurg. 71: 105 (1989)). Additionally, the controlled release system can be placed in proximity of the therapeutic target (e.g., a tumor), thus requiring only a fraction of the systemic dose. See, for example, Goodson, In: Medical Applications of Controlled Release, 1984. (CRC Press, Boca Raton, Fla.).

The amount of the agents which will be effective in the treatment of a particular disorder or condition will depend on the nature of the disorder or condition, and may be determined by standard clinical techniques by those of skill within the art. In addition, in vitro assays may optionally be employed to help identify optimal dosage ranges. The precise dose to be employed in the formulation will also depend on the route of administration, and the overall seriousness of the disease or disorder, and should be decided according to the judgment of the practitioner and each patient's circumstances. Ultimately, the attending physician will decide the amount of the agent with which to treat each individual patient. In certain embodiments, the attending physician will administer low doses of the agent and observe the patient's response. Larger doses of the agent may be administered until the optimal therapeutic effect is obtained for the patient, and at that point the dosage is not increased further. In general, the daily dose range of a drug lie within the range known in the art for a particular drug or biologic. Effective doses may be extrapolated from dose-response curves derived from in vitro or animal model test systems. Ultimately the attending physician will decide on the appropriate duration of therapy using compositions of the present invention. Dosage will also vary according to the age, weight and response of the individual patient.

Methods for administering antibodies for therapeutic use is well known to one skilled in the art. In certain embodiments, small particle aerosols of antibodies or fragments thereof may be administered (see e.g., Piazza et al., J. Infect. Dis., Vol. 166, pp. 1422-1424, 1992; and Brown, Aerosol Science and Technology, Vol. 24, pp. 45-56, 1996). In certain embodiments, antibodies are administered in metered-dose propellant driven aerosols. In certain embodiments, antibodies may be administered in liposomes, i.e., immunoliposomes (see, e.g., Maruyama et al., Biochim. Biophys. Acta, Vol. 1234, pp. 74-80, 1995). In certain embodiments, immunoconjugates, immunoliposomes or immunomicrospheres containing an agent of the present invention is administered by inhalation.

In certain embodiments, antibodies may be topically administered to mucosa, such as the oropharynx, nasal cavity, respiratory tract, gastrointestinal tract, eye such as the conjunctival mucosa, vagina, urogenital mucosa, or for dermal application. In certain embodiments, antibodies are administered to the nasal, bronchial or pulmonary mucosa. In order to obtain optimal delivery of the antibodies to the pulmonary cavity in particular, it may be advantageous to add a surfactant such as a phosphoglyceride, e.g. phosphatidylcholine, and/or a hydrophilic or hydrophobic complex of a positively or negatively charged excipient and a charged antibody of the opposite charge.

Other excipients suitable for pharmaceutical compositions intended for delivery of antibodies to the respiratory tract mucosa may be a) carbohydrates, e.g., monosaccharides such as fructose, galactose, glucose. D-mannose, sorbiose, and the like; disaccharides, such as lactose, trehalose, cellobiose, and the like; cyclodextrins, such as 2-hydroxypropyl-β-cyclodextrin; and polysaccharides, such as raffinose, maltodextrins, dextrans, and the like; b) amino acids, such as glycine, arginine, aspartic acid, glutamic acid, cysteine, lysine and the like; c) organic salts prepared from organic acids and bases, such as sodium citrate, sodium ascorbate, magnesium gluconate, sodium gluconate, tromethamine hydrochloride, and the like: d) peptides and proteins, such as aspartame, human serum albumin, gelatin, and the like; e) alditols, such mannitol, xylitol, and the like, and f) polycationic polymers, such as chitosan or a chitosan salt or derivative.

For dermal application, the antibodies of the present invention may suitably be formulated with one or more of the following excipients: solvents, buffering agents, preservatives, humectants, chelating agents, antioxidants, stabilizers, emulsifying agents, suspending agents, gel-forming agents, ointment bases, penetration enhancers, and skin protective agents.

Examples of solvents are e.g. water, alcohols, vegetable or marine oils (e.g. edible oils like almond oil, castor oil, cacao butter, coconut oil, corn oil, cottonseed oil, linseed oil, olive oil, palm oil, peanut oil, poppy seed oil, rapeseed oil, sesame oil, soybean oil, sunflower oil, and tea seed oil), mineral oils, fatty oils, liquid paraffin, polyethylene glycols, propylene glycols, glycerol, liquid polyalkylsiloxanes, and mixtures thereof.

Examples of buffering agents are e.g. citric acid, acetic acid, tartaric acid, lactic acid, hydrogenphosphoric acid, diethyl amine etc. Suitable examples of preservatives for use in compositions are parabenes, such as methyl, ethyl, propyl p-hydroxybenzoate, butylparaben, isobutylparaben, isopropylparaben, potassium sorbate, sorbic acid, benzoic acid, methyl benzoate, phenoxyethanol, bronopol, bronidox, MDM hydantoin, iodopropynyl butylcarbamate, EDTA, benzalconium chloride, and benzylalcohol, or mixtures of preservatives.

Examples of humectants are glycerin, propylene glycol, sorbitol, lactic acid, urea, and mixtures thereof.

Examples of antioxidants are butylated hydroxy anisole (BHA), ascorbic acid and derivatives thereof, tocopherol and derivatives thereof, cysteine, and mixtures thereof.

Examples of emulsifying agents are naturally occurring gums, e.g. gum acacia or gum tragacanth; naturally occurring phosphatides, e.g. soybean lecithin, sorbitan monooleate derivatives: wool fats; wool alcohols; sorbitan esters; monoglycerides; fatty alcohols; fatty acid esters (e.g. triglycerides of fatty acids); and mixtures thereof.

Examples of suspending agents are e.g. celluloses and cellulose derivatives such as, e.g., carboxymethyl cellulose, hydroxyethylcellulose, hydroxypropylcellulose, hydroxypropylmethylcellulose, carraghenan, acacia gum, arabic gum, tragacanth, and mixtures thereof.

Examples of gel bases, viscosity-increasing agents or components which are able to take up exudate from a wound are: liquid paraffin, polyethylene, fatty oils, colloidal silica or aluminum, zinc soaps, glycerol, propylene glycol, tragacanth, carboxyvinyl polymers, magnesium-aluminum silicates, Carbopol®, hydrophilic polymers such as, e.g. starch or cellulose derivatives such as, e.g., carboxymethylcellulose, hydroxyethylcellulose and other cellulose derivatives, water-swellable hydrocolloids, carragenans, hyaluronates (e.g. hyaluronate gel optionally containing sodium chloride), and alginates including propylene glycol alginate.

Examples of ointment bases are e.g. beeswax, paraffin, cetanol, cetyl palmitate, vegetable oils, sorbitan esters of fatty acids (Span), polyethylene glycols, and condensation products between sorbitan esters of fatty acids and ethylene oxide, e.g. polyoxyethylene sorbitan monooleate (Tween).

Examples of hydrophobic or water-emulsifying ointment bases are paraffins, vegetable oils, animal fats, synthetic glycerides, waxes, lanolin, and liquid polyalkylsiloxanes. Examples of hydrophilic ointment bases are solid macrogols (polyethylene glycols). Other examples of ointment bases are triethanolamine soaps, sulphated fatty alcohol and polysorbates.

Examples of other excipients are polymers such as carmelose, sodium carmelose, hydroxypropylmethylcellulose, hydroxyethylcellulose, hydroxypropylcellulose, pectin, xanthan gum, locust bean gum, acacia gum, gelatin, carbomer, emulsifiers like vitamin E, glyceryl stearates, cetanyl glucoside, collagen, carrageenan, hyaluronates and alginates and chitosans.

The dose of antibody required in humans to be effective in the treatment cancer differs with the type and severity of the cancer to be treated, the age and condition of the patient, etc. Typical doses of antibody to be administered are in the range of 1 μg to 1 g, preferably 1-1000 μg, more preferably 2-500, even more preferably 5-50, most preferably 10-20 μg per unit dosage form. In certain embodiments, infusion of antibodies of the present invention may range from 10-500 mg/m².

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transduction with viral (typically lentivirus, adeno associated virus (AAV) and adenovirus) vectors.

In certain embodiments, an agent that reduces a gene signature as described herein is used to treat a subject in need thereof having a cancer. In certain embodiments, an agent that reduces an immunotherapy resistance signature is co-administered with an immunotherapy or is administered before administration of an immunotherapy. The immunotherapy may be adoptive cell transfer therapy, as described herein or may be an inhibitor of any check point protein described herein. Specific check point inhibitors include, but are not limited to anti-CTLA4 antibodies (e.g., Ipilimumab), anti-PD-1 antibodies (e.g., Nivolumab, Pembrolizumab), and anti-PD-L1 antibodies (e.g., Atezolizumab).

In another aspect, provided is a pharmaceutical pack or kit, comprising one or more containers filled with one or more of the ingredients of the pharmaceutical compositions.

In another aspect, provided is a kit for detecting the gene signature as described herein.

The invention is further described in the following examples, which do not limit the scope of the invention described in the claims.

### EXAMPLES

### Example 1—Description of Study

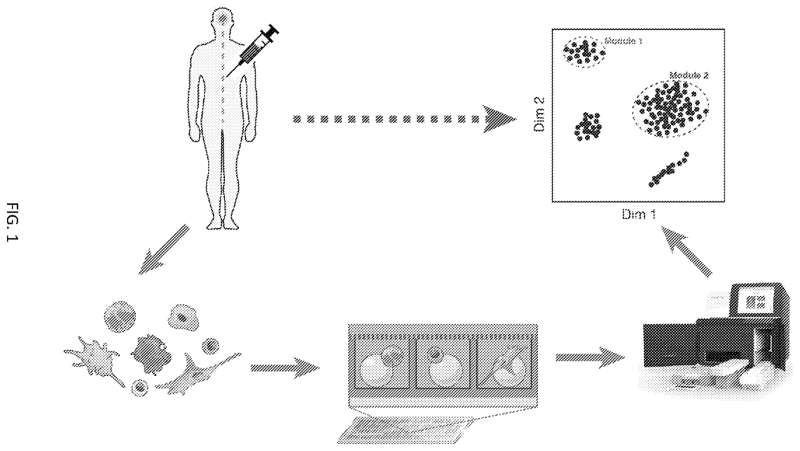

Applicants applied Seq-Well to cerebrospinal fluid liquid biopsies from patients with LMD collected as part of a phase II clinical trial of the immune checkpoint inhibitor pembrolizumab. Pembrolizumab is an antibody which targets PD1, preventing its binding to ligand PDL1. Applicants used Seq-Well to profile the phenotypes of cells in the microenvironment of LMD with single-cell resolution (FIG. 1). Collectively, the work establishes a novel, extendable single-cell genomics paradigm for utilizing liquid biopsies to help guide new therapeutic and diagnostic strategies for diseases in the central nervous system.

Briefly, 18 patients with LMD from breast cancer are enrolled in a phase II clinical trial of pembrolizumab, an immune checkpoint inhibitor, which antagonizes PD-1/PD-L1 interaction. The CSF from these patients is spun down, and a single-cell suspension generated for Seq-Well. Importantly, the number of cells in the CSF from clinical liquid biopsies is very low (<5,000 cells) and cannot be flow sorted, enriched, or otherwise manipulated to achieve single-cell capture except through the Seq-Well pipeline.

Following Seq-Well, the resulting cDNA is prepared for sequencing using an Illumina Nextera XT kit and sequenced on a NextSeq 500/550. Samples were demultiplexed with bcl2fastq and aligned to hg19 using the STAR aligner. Single-cell CSF samples were sequenced to an average depth of 66,500 reads per cell with an average complexity of ˜1200 genes per cell. In total, 7 patients were profiled with detected LMD and who were on the phase II pembrolizumab trial and one additional patient with brain metastases but not LMD was profiled (Patient 011). 4 patients had at least one pre-treatment sample and at least one post-treatment sample (3 of the patients had 2 post-treatment sample), making this one of the first single-cell RNA-Seq studies of a neoplastic disease pre/post-treatment. Additionally, Applicants profiled 2 patients with only pre-treatment samples and 1 patient with a single post-treatment sample. Of the patients in the study who had LMD, three of the patients exceeded the threshold survival for response in the phase II clinical trial (P022, P037, and P043) whereas three of the patients did not (P014, P029, P046). Importantly, one of the patients (P001) was subject to intrathecal herceptin treatment prior to starting the pembrolizumab trial. This patient's pre-treatment samples were not included in the response analysis.

### Example 2—Results

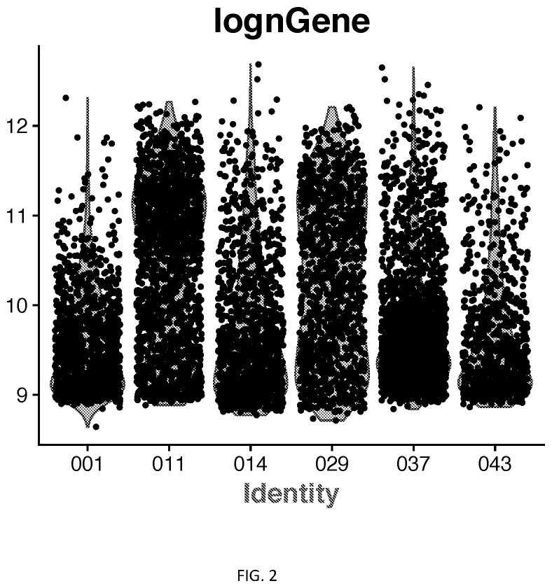

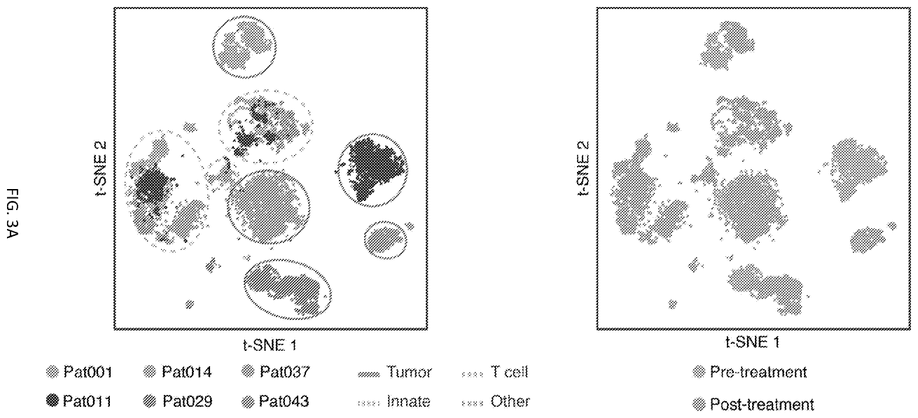

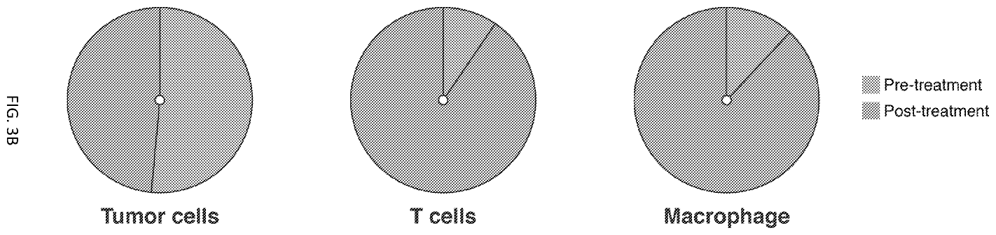

Following preprocessing of end-counted single-cell RNA-Sequencing data by standard methods, a total of 8577 single-cell transcriptomes across 7 patients were detected to an average complexity of 1200 genes per cell (FIG. 2). The results demonstrate the power of Seq-Well to capture transcriptomic information at from a reservoir otherwise impossible to profile at single-cell resolution. The data are also among the first single-cell RNA-Sequencing data of cancer systems profiled pre- and post-treatment.

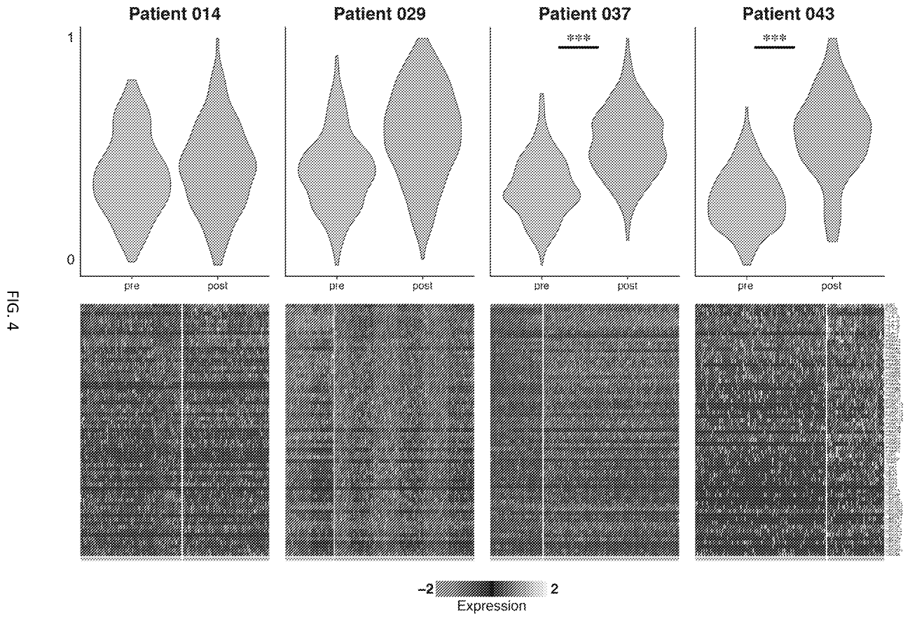

Single-cell RNA-Sequencing data from CSF samples described above was analyzed by principal components analysis followed by non-linear stochastic neighbor embedding by t-SNE. The resulting 2-dimensional map revealed a rich variety of cellular information regarding the progression of LMD following treatment of PD1-inhibitor (FIG. 3A). In particular, Applicants found that the tumor cells resided in distinct, patient-specific clusters that were uniquely distributed across the reduced-dimension space (see grey circled clusters in figure above). In contrast, the immune populations (see dotted circled clusters in figure above) contained mixtures of cells from all patients and clustered instead by broad immune subset (innate vs. adaptive immune) rather than by patient. This result is consistent with previous findings in the single-cell RNA-Sequencing field from patient-derived tumor biopsies (in particular Tirosh et al, Science, 2016).

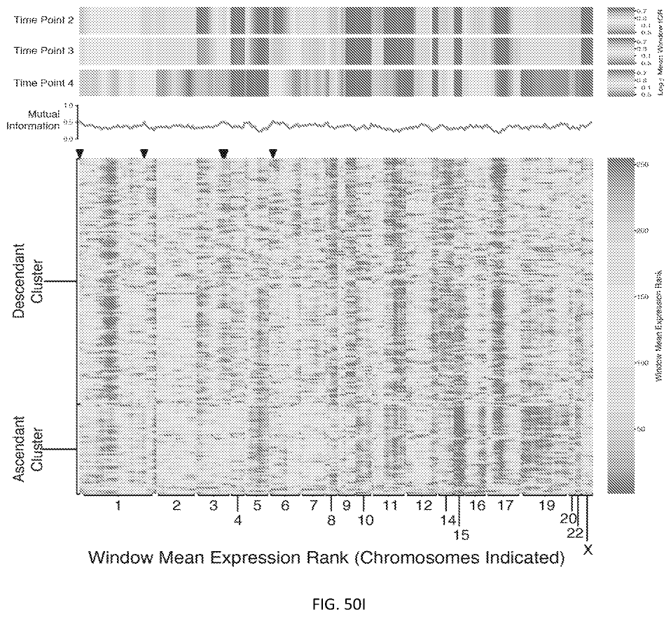

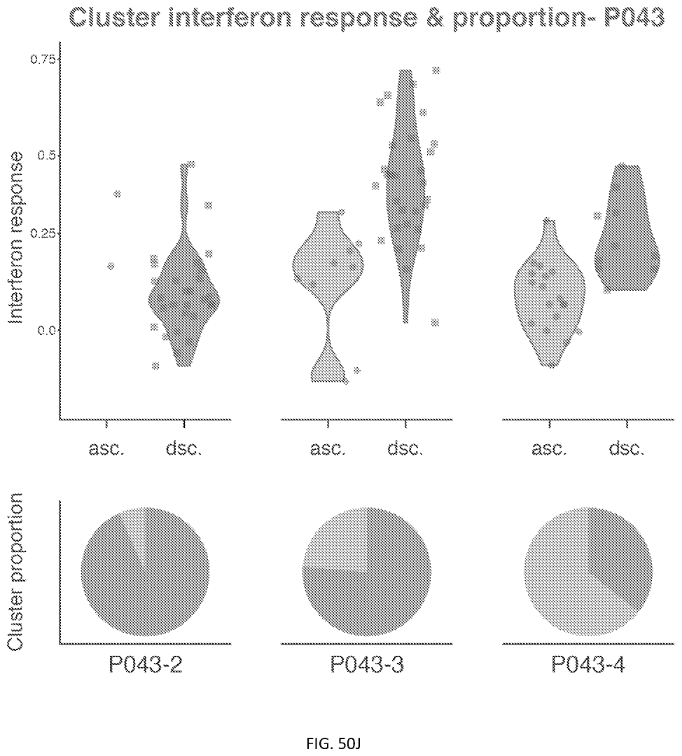

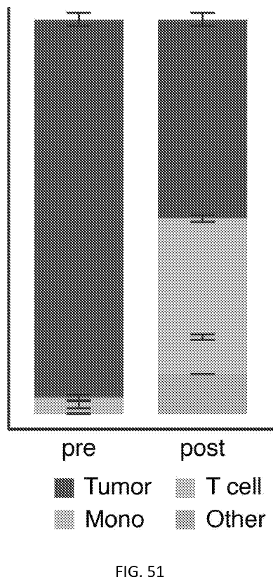

Delineating single-cell transcriptomes by treatment status of patient during CSF collection reveals important bias in cell type representation (FIG. 3B). Although tumor populations shift in numbers, there is no discernably consistent change in absolute numbers of tumor cells between pre- and post-treatment of the same patient. However, the vast majority of immune cells (>90%) were present only after treatment with pembrolizumab (FIG. 51). This result is conserved across all patients, and demonstrates the efficacy of the study to detect wide-sweeping changes in the tumor microenvironment as a result of treatment.

To investigate treatment-induced effects on the tumor populations, Applicants focused on patients with enough tumor cells pre- and post-treatment for whom Applicants were powered to make comparisons. In total, four patients met this criteria—2 patients who did not meet threshold survival (P014 and P029) and 2 patients who met or exceeded threshold (P037 and P043). Applicants performed differential expression by likelihood-ratio test using a 3-parameter bimodal distribution to describe single-cell transcriptomic data. Applicants ran the analysis between the tumor cells pre- and post-treatment for these four patients, controlling for changes in complexity and adjusting p-values based on significance power.

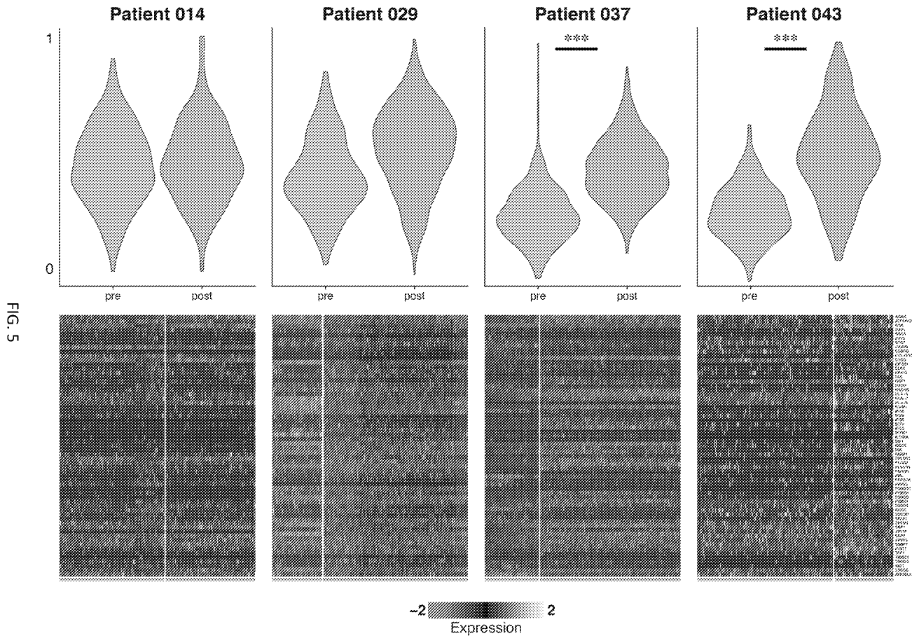

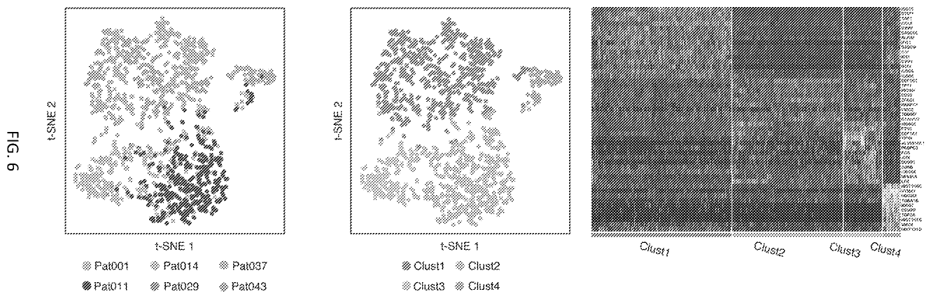

Applicants found that tumor cells from patients with increased survival exhibited statistically significant increased expression of antigen presentation machinery following treatment with pembrolizumab. In contrast, Applicants found that tumor cells from patients without increased survival did not exhibit the same increased expression of antigen presentation machinery (FIG. 4).

Importantly, upregulation of antigen presentation, particularly by MHC class I on tumor cells is the primary mechanism by which activated T cells can initiate tumor cell killing. The PD1-inhibitor pembrolizumab is believed to reverse attenuation of cytotoxic T cell response by PD1, and so upregulation of antigen presentation is a consistent observation with the activity of the drug.

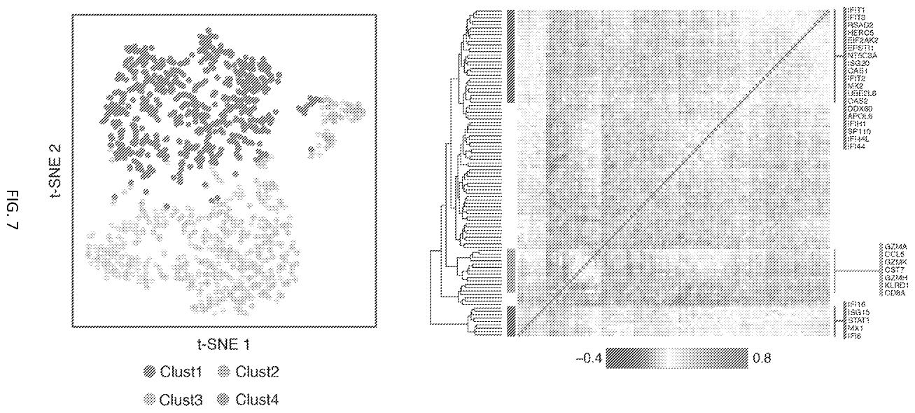

Furthermore, Applicants found that the tumor cells in patients with increased survival exhibited upregulation of interferon stimulated genes (ISGs) (FIG. 5). Among these genes were, importantly, genes such as STAT1, B2M, and NMI, which are genes important for the induction of interferon gamma and eventually result in antigen presentation induced by interferon sensing. These features are again not found in the tumor cells of patients without increased survival. The gene signatures for interferon response and antigen presentation for all four patients are correlated very strongly (0.9>r2>0.5) above a background developed by permutation algorithm (n=1000). The data strongly suggest that increased interferon (and specifically interferon gamma) in the tumor microenvironment is responsible for increased antigen presentation.

With the evidence that improved antigen presentation in tumor cells from patients with improved survival derives from increased interferon levels in the tumor microenvironment of the CSF, Applicants investigated immune compartments to understand how these microenvironmental shifts may manifest.

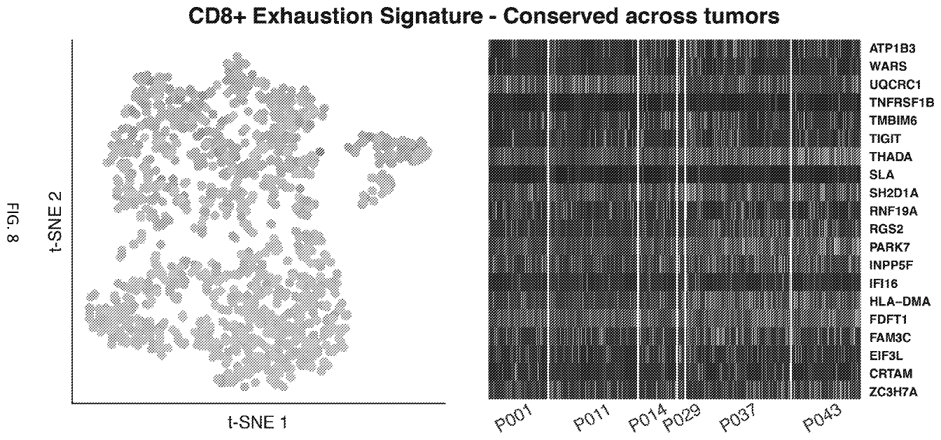

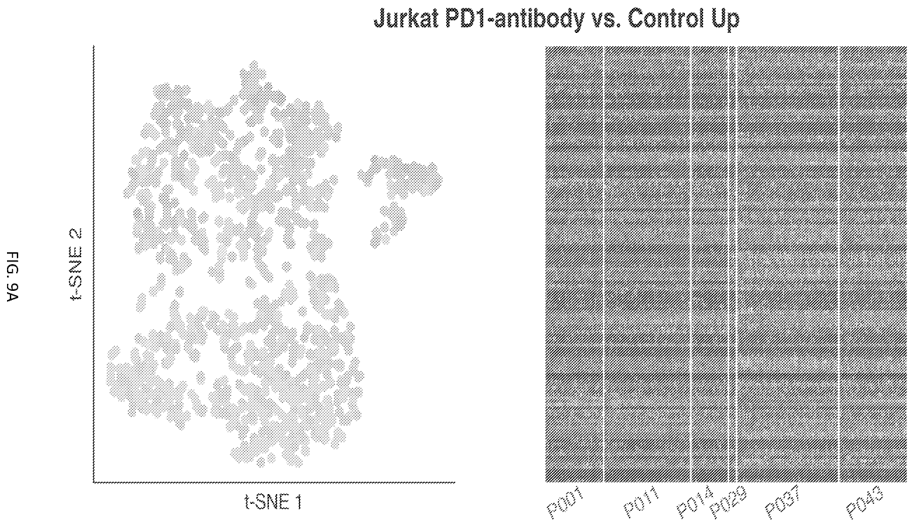

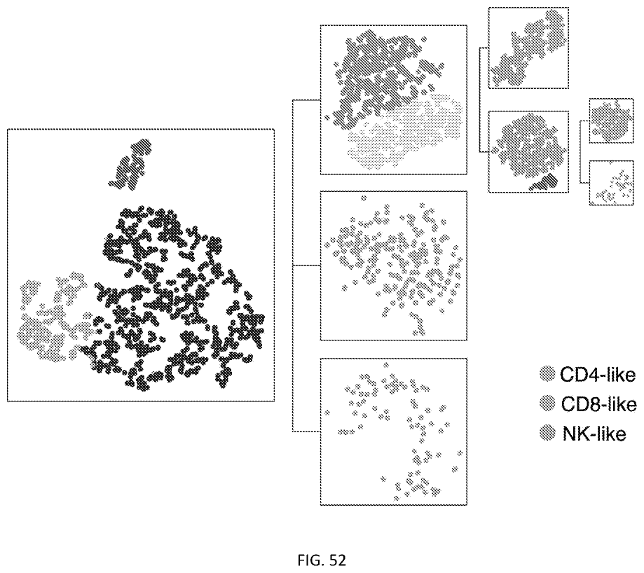

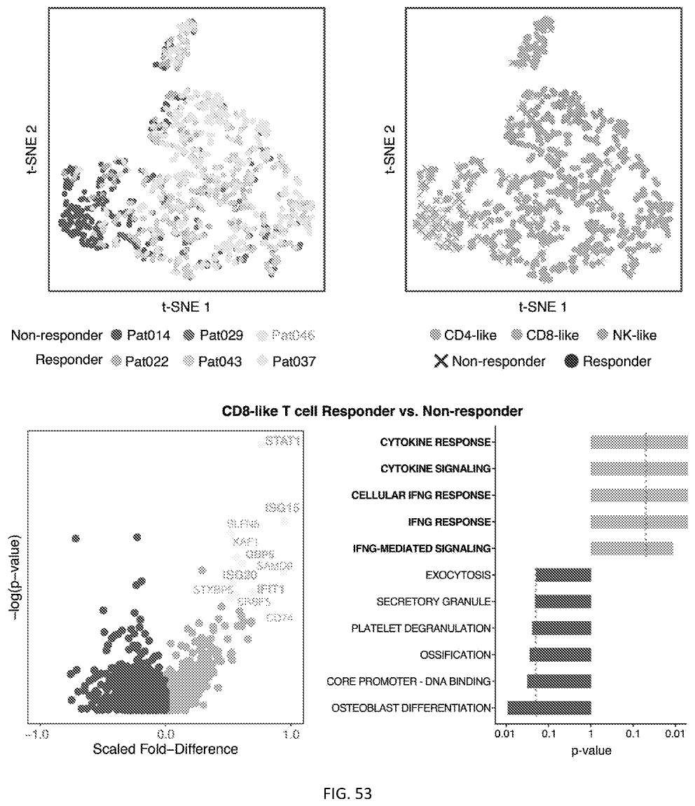

Applicants analyzed the adaptive immune “compartment” of the single-cell RNA-Seq data across all patients using the same dimensional reduction strategy applied to the entire dataset (FIG. 6). In total, Applicants analyzed 1,467 transcriptomes across six patients. Applicants note that one of these patients (P001) had intrathecal herceptin prior to administration of pembrolizumab. P001 is also the only patient who exhibited T cells pre-treatment. T cell transcriptomes were iteratively analyzed with dimensional reduction and clustering to determine subsets (FIG. 52). Following completion, differential expression between T cells of the same subset between responding and non-responding patients demonstrated conserved gene expression shifts. In particular, Applicants note upregulated interferon signaling and sensing pathways in responding patient T cells (FIG. 53).

Clustering by kmeans clustering following principal components analysis reveals 4 distinct populations of T cells distributed in a biased fashion across patients (FIG. 6). In particular, one of these populations consists almost entirely of cells from long surviving patients (043 and 037—clust1). Differential expression as described above reveals distinct genes that define the subpopulations of T cells. In particular, Applicants find a cluster consisting of cytotoxic cells (clust1), CD4-like cells (clust2), a cluster entirely made up of pre-treatment T cells (clust3) and a cluster of proliferating T cells (clust4). All patients contain a small proportion of proliferating T cells, but only long-surviving patients were represented by in the cytotoxic cluster. Furthermore, the analysis reveals important interferon-regulatory behavior in the T cells from these patients.

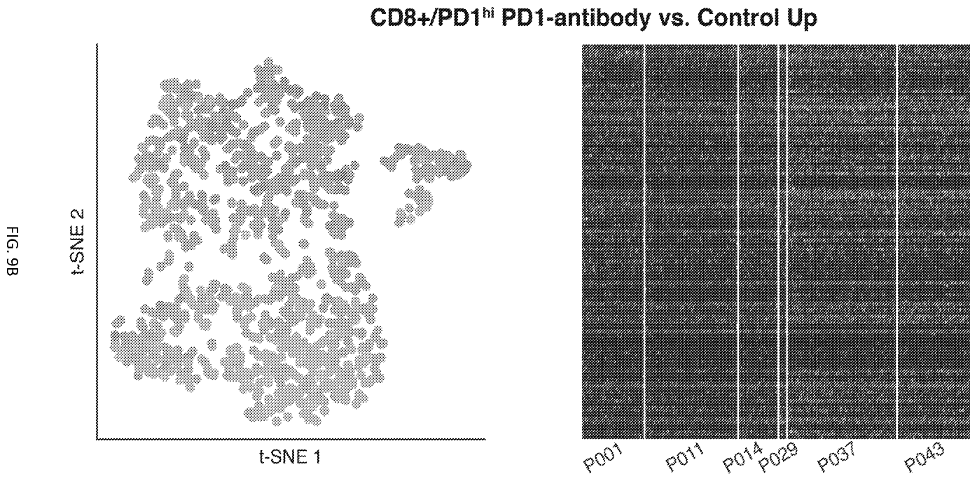

Applicants took all genes from the differential expression results that exhibited a log FC>0.5 specific to clust1 and ran a gene-by-gene correlation analysis, examining only over cells in clust1. This analysis revealed the co-expression of programs relating to cytotoxicity and the regulation of interferon production and sensitivity in T cells (see figure below; orange block=cytotoxic, purple blocks=IFN) (FIG. 7).

These data strongly suggest that the interferon regulatory network of T cells in long-surviving patients is activated (or perhaps hyper-activated), whereas this does not appear to be the case in the other patients. Applicants note also that P001 is an additional long-surviving patient, but this patient had resident T cells in the CSF previous to pembrolizumab treatment and was also subject to multiple treatment regimens.

Applicants analyzed the T cell transcriptomes using previously curated gene lists of exhaustion and PD1/antibody activity. First, Applicants used the list of conserved genes reported in Tirosh, et al, Science, 2016 for T cell exhaustion that was orthogonal to cytotoxicity. Applicants found that of the genes in this list that were detected in the data, T cells from long-surviving patients exhibited increased expression of these genes (shown below, in heatmap and as a feature plot of signature derived from these genes) (FIG. 8).

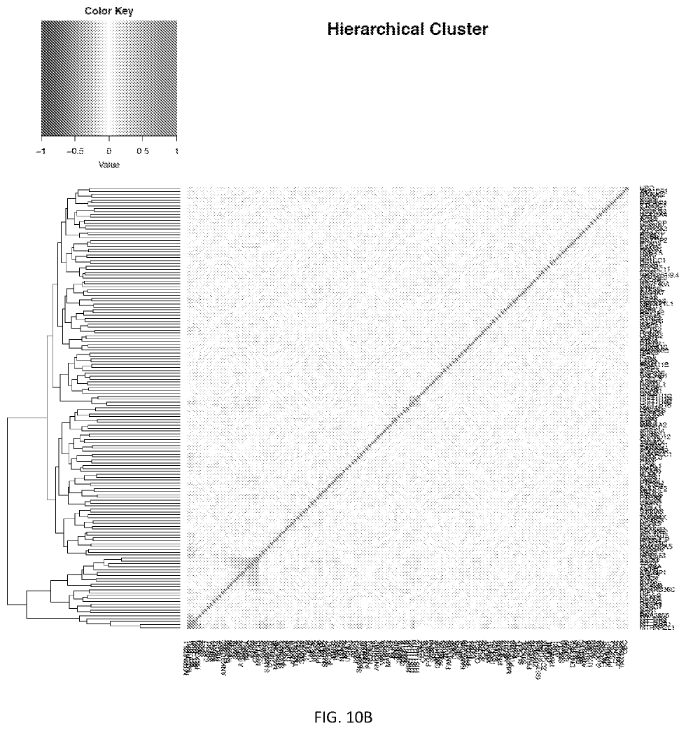

Additionally, Applicants mined data from a publication by Nick Haining (Harvard) and John Wherry (Penn) describing the induction of T cells (Jurkat cell line, CD8 T cells) with beads containing antibodies against PD1. The data demonstrate that genes which were upregulated when T cells were incubated with PD1-antibody are also upregulated in T cells from patients who are long-survivors (FIG. 9). These data suggest that patients who are long survivors contain, within their CSF, T cell populations, which have interacted with pembrolizumab (a PD1-antibody) and exhibit exhaustion-like behavior.

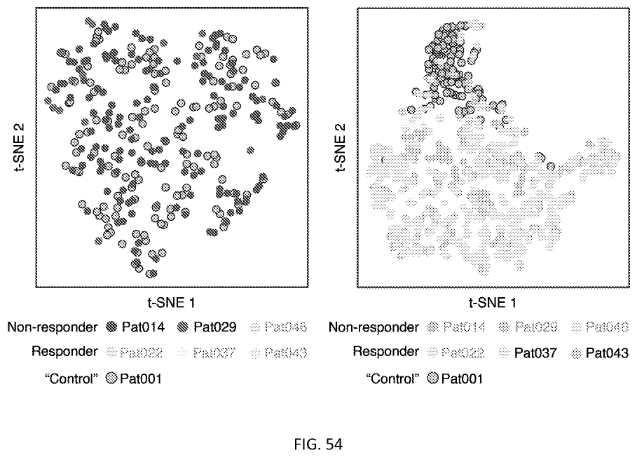

Applicants also observed that pre-treatment T cells appeared to cluster with non-responding T cells (FIG. 54). T cells from Patient 001 (the only patient with pre-treatment T cells, but who never received pembrolizumab treatment) were individually clustered with T cells from non-responding patients and responding patients. Applicants note that in reduced dimension space (visualizing all significant principal components), the T cells from P001 do not appear to cluster separately from the non-responding patient cells. However, when clustered with T cells from responding patients, T cells from P001 uniquely cluster away from all other cells, suggesting the difference in phenotype between P001 T cells and responding patient T cells are more pronounced than those between non-responding patients.

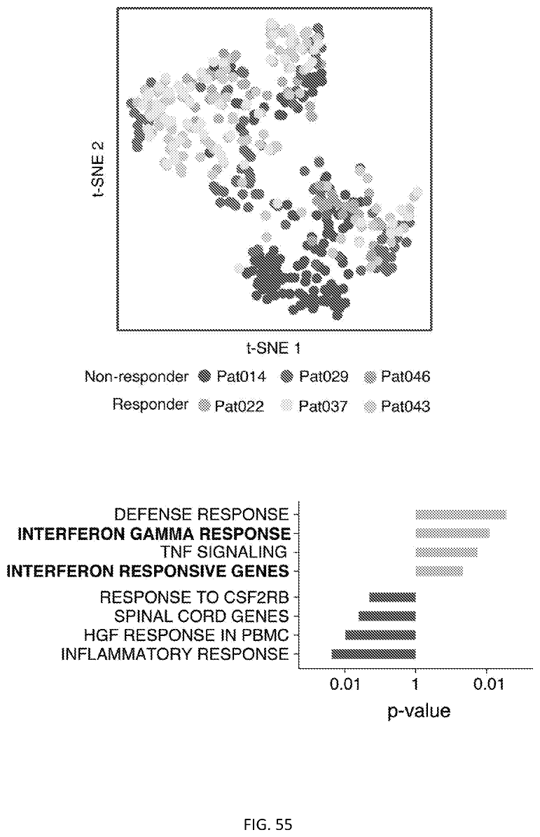

Analysis of the innate immune population suggests an orchestrated immune response (FIG. 55). Using a similar analysis pipeline as previously presented, Applicants investigated the extent of the shift in interferon-based response to pembrolizumab across cell types of the immune compartment in LMD. In particular, Applicants show that the innate immune compartment, particularly the monocyte/macrophage population shows strong difference in phenotype between responding and non-responding patients. Differential expression between the groups that consist of cells from these patients reveals upregulation of interferon gamma response in this population as well. Furthermore, Applicants find specific upregulation of interferon machinery including DDX58, IRF1, and other transcription factors, suggesting an active interferon feedback loop present in these patients.

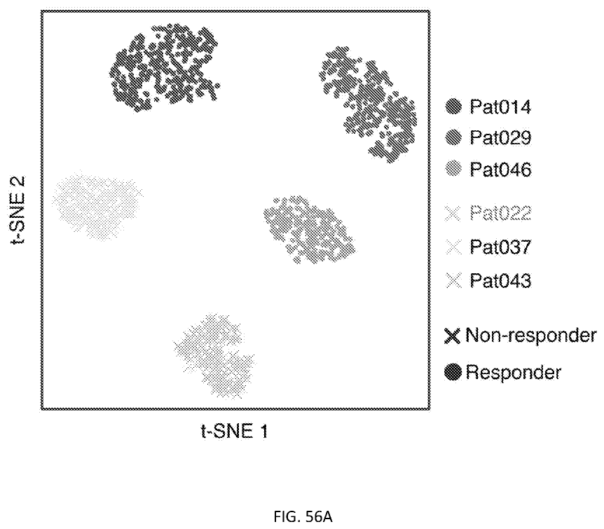

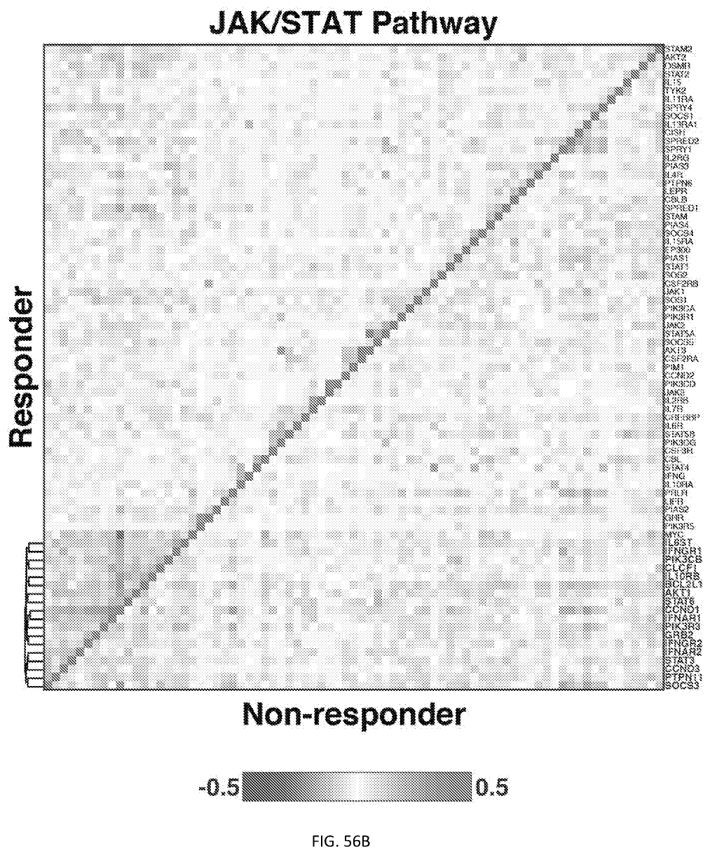

Analysis of pre-treatment tumor cells suggests disruption of JAK/STAT pathway underlies response (FIG. 56). Applicants sought to understand the upstream drivers that affect patients' ability to respond to pembrolizumab treatment with the ability to present antigen. In particular, Applicants sought to distinguish between differences in gene expression and pathway regulation between these groups. Therefore, Applicants applied differential coexpression to dissect whether pathways of interest are differentially regulated, determined by comparing the correlation structure of genes in these pathways.

Applicants found that of candidate pathways that are known to affect the efficacy of checkpoint blockade inhibitor therapy, the JAK/STAT pathway exhibited particular differences in correlation structure between responding and non-responding patients. In particular, there exists strong correlation structure among genes that regulate interferon induction and response. This structure disappears in the non-responding patients, suggesting that a regulatory network including these genes may be impaired in these patients. Dysfunction of the JAK/STAT pathway has previously been shown to strongly affect T cell mediated killing of tumor cells, antigen presentation in cancer, and recognition of checkpoint blockade antagonists (Shin, et al. Cancer Discovery. 2017; Sade-Feldman, et al. Nature Communications. 2017).

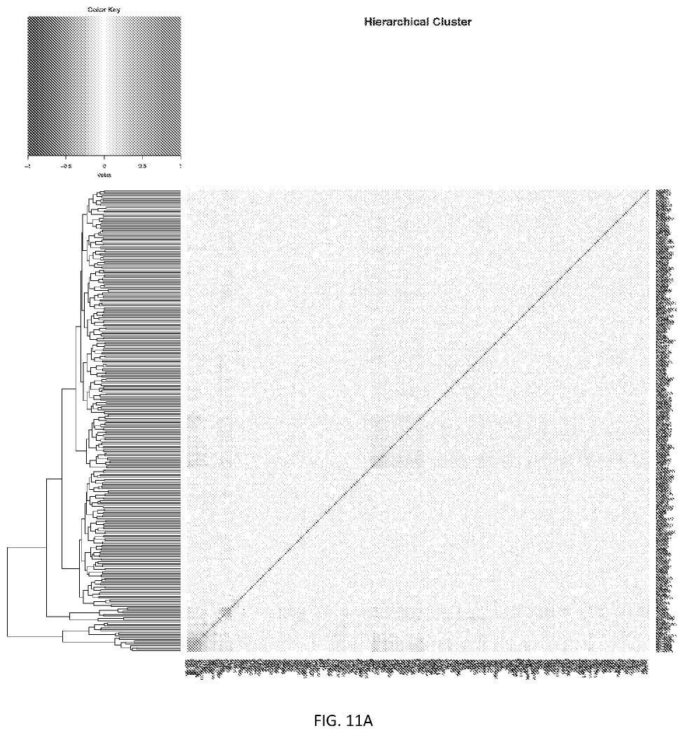

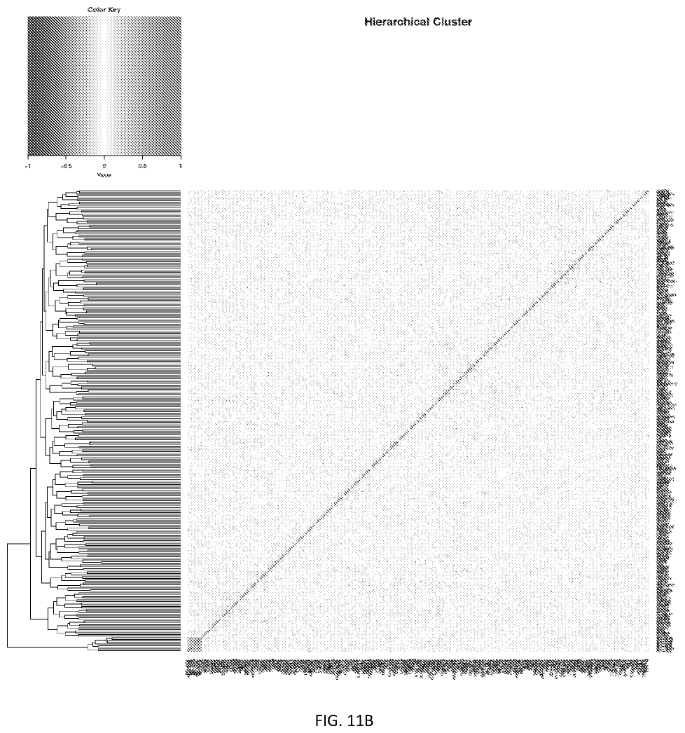

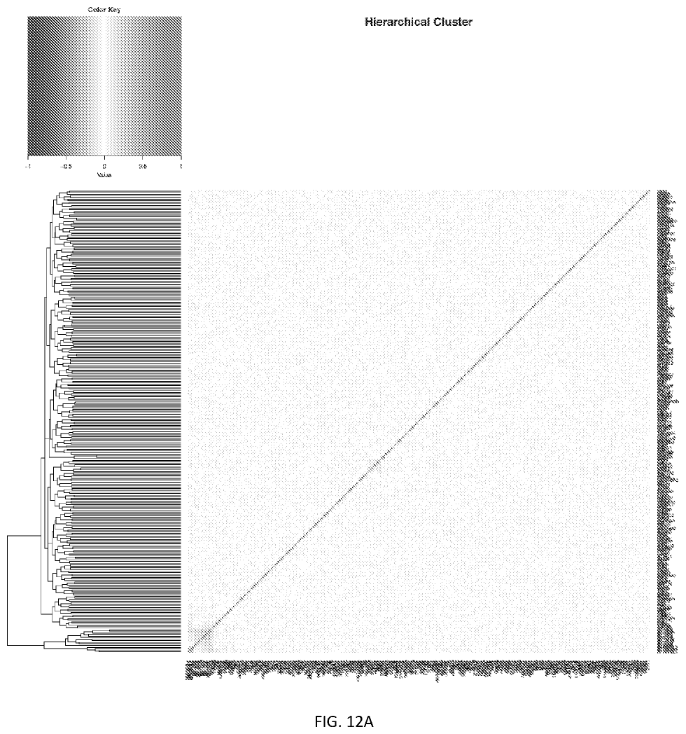

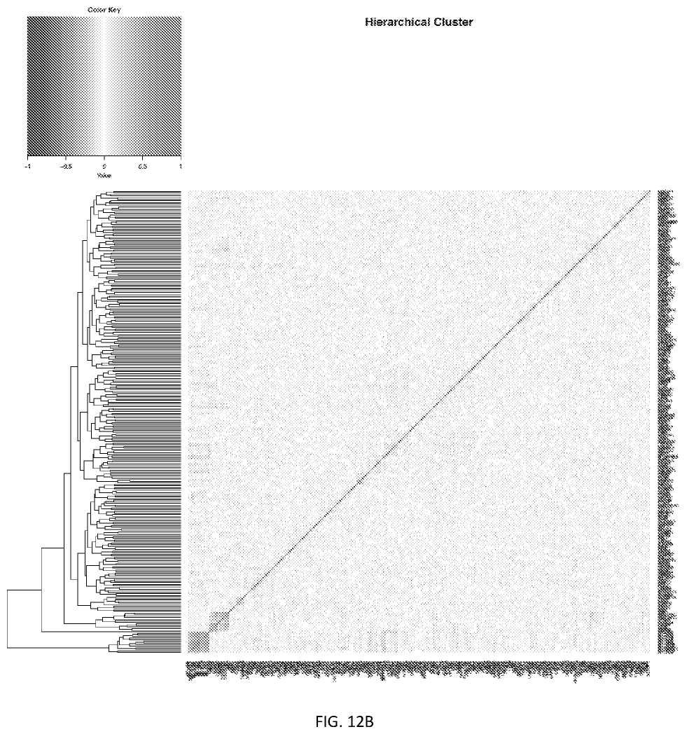

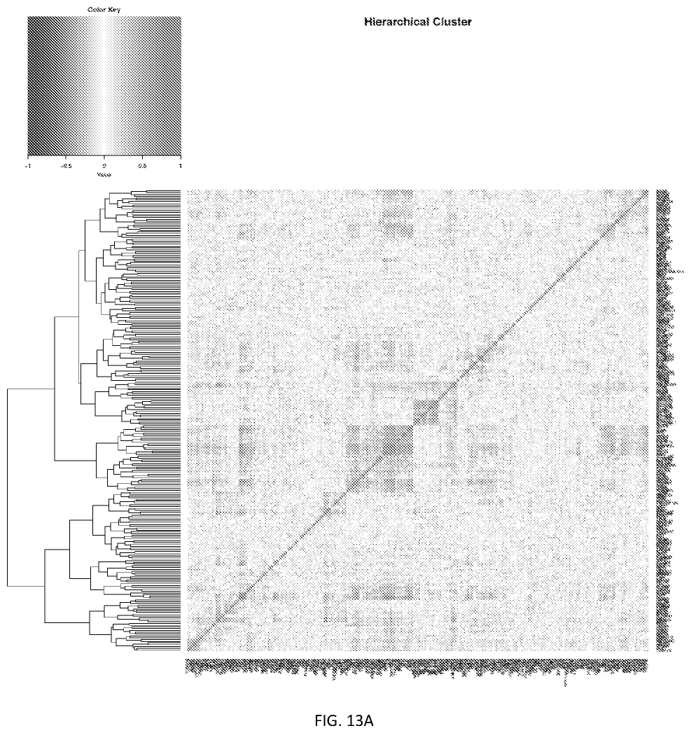

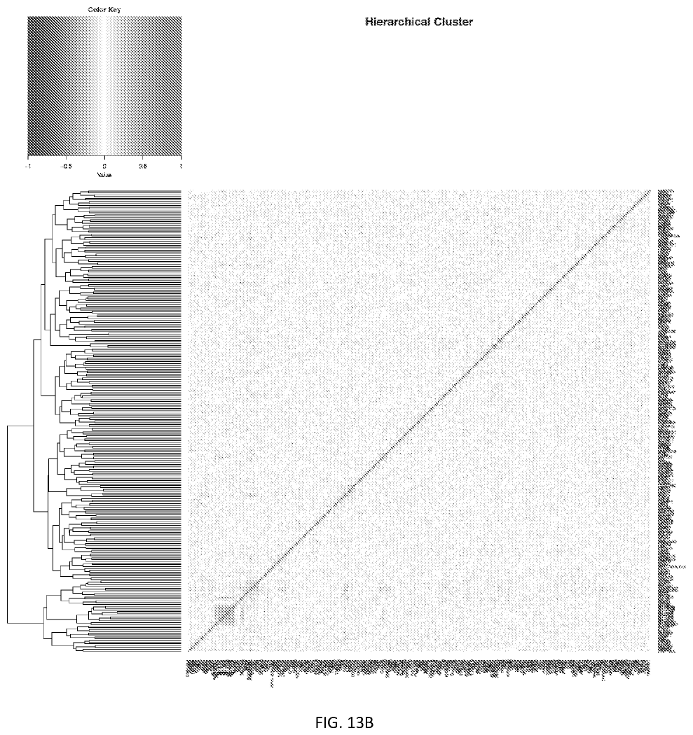

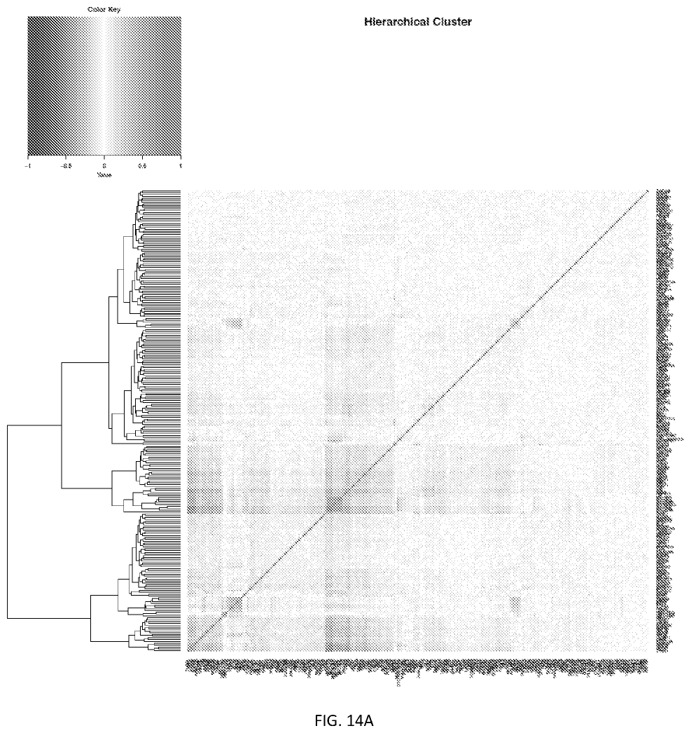

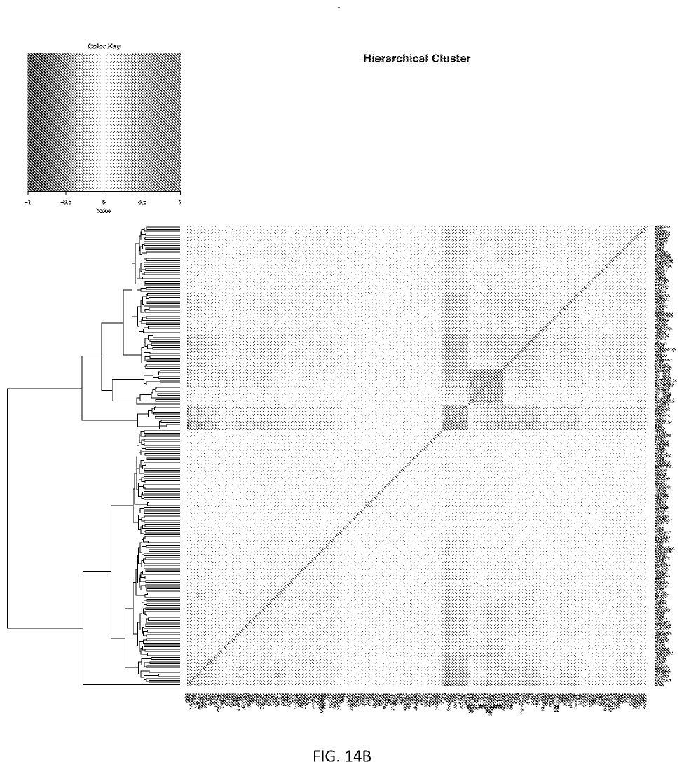

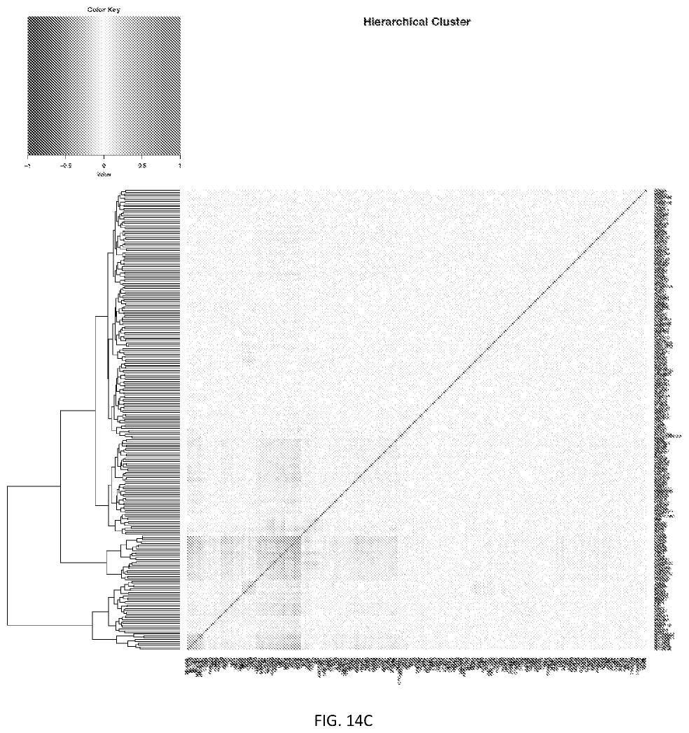

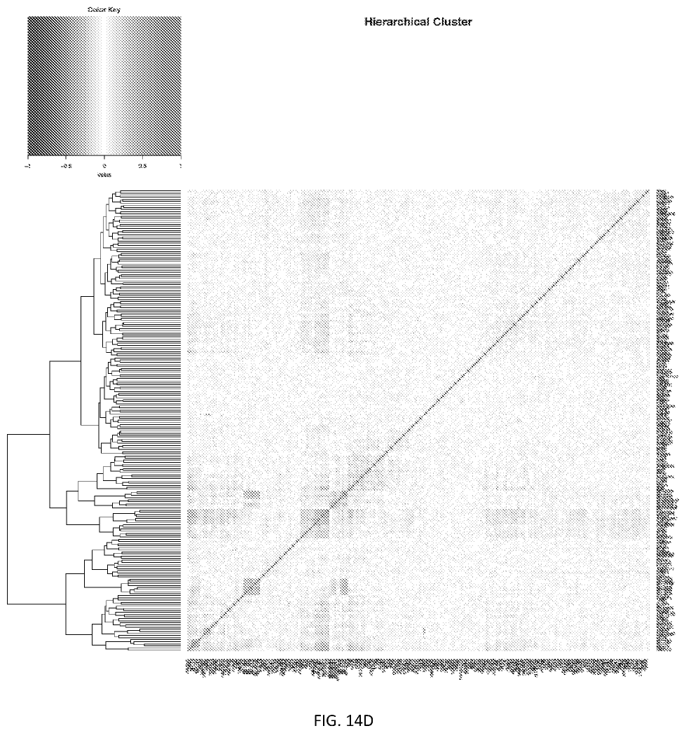

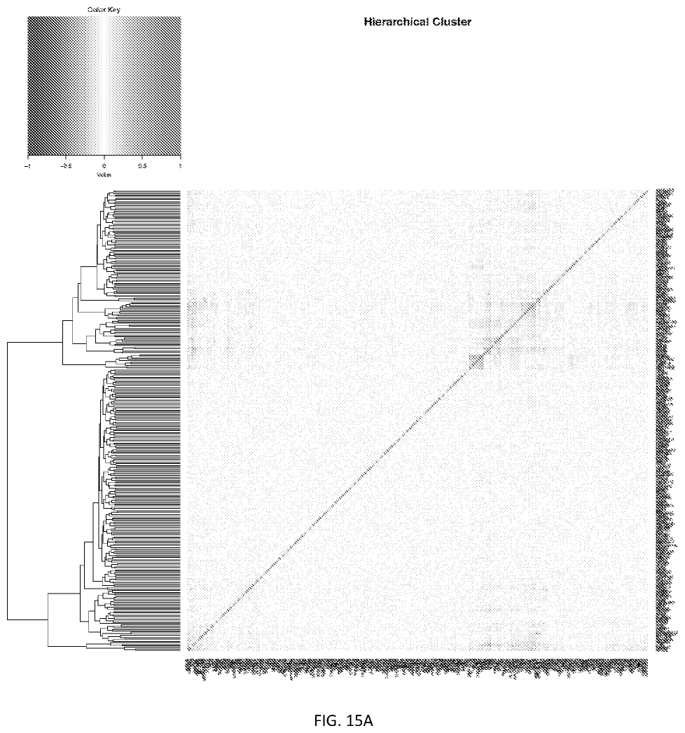

Applicants also performed additional analyses of differential correlation (FIGS. 10-19). The analysis performed was: overlapping variable genes from each subset of cells between the two groups compared were found and analyzed using a genes x genes correlation. As used herein, correlation may refer to genes that go up together or down together in regards to gene expression. This correlation was calculated for both subsets of cells and then used to determine shared and unique clusters of genes to describe active modules with tightened correlation structure at the single-cell level. Post-treatment shows tightening of cycling-associated genes (FIG. 10). Post-treatment shows tightening of cycling-associated genes and some tightening of antigen-presentation machinery (FIG. 11). Tightening of antigen-presentation machinery is evident (FIG. 12). There is a significant increase in antigen-presentation and interferon-response machinery in post-treatment (FIG. 13). In Post treatment patients Applicants observe dramatic shifts in the correlation matrices (FIG. 14). In particular Applicants observe shifts in tightening of correlation for proliferation post-treatment. This is consistent with previously described up regulation of a proliferating subset of tumor cells. However, note that this data is taken as bulk across multiple patients in post-treatment. In Pre treatment samples Applicants observe increased tightening of correlation of interferon networks pre-treatment in non-responding patients, relative to responding patients (FIG. 14). Applicants observe up regulation of cycling and up regulation of granzyme genes associated with cytotoxicity, but because of the heterogeneity in the populations and the difference between responders and non-responders, that comparison is likely more valuable (FIG. 15). Applicants again observed up regulation of cycling and up regulation of granzyme genes associated with cytotoxicity (FIG. 16). Applicants observe some evidence of tightening of the interferon network, but those genes were not as represented in this initial data set due to the fact that Applicants took overlapping variable genes. When included in a more targeted analysis, they are more correlated in responding patients.

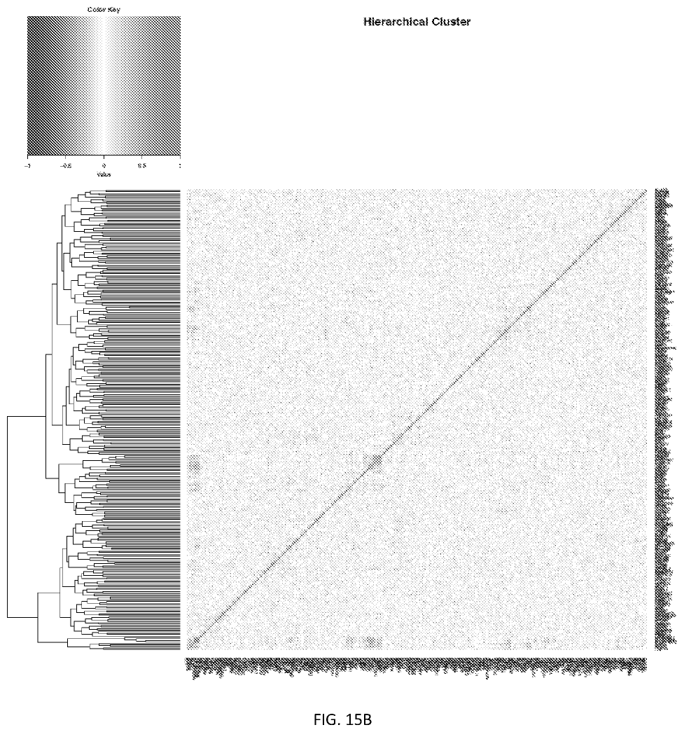

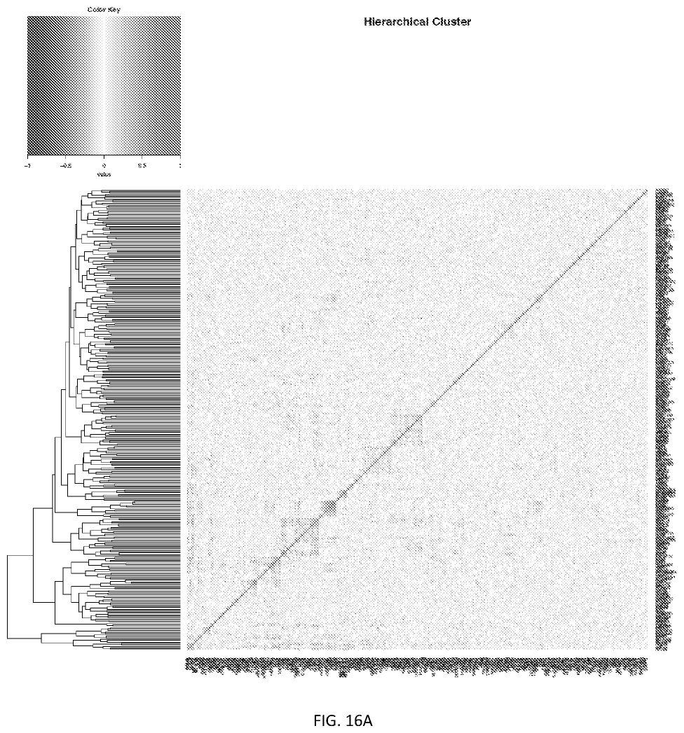

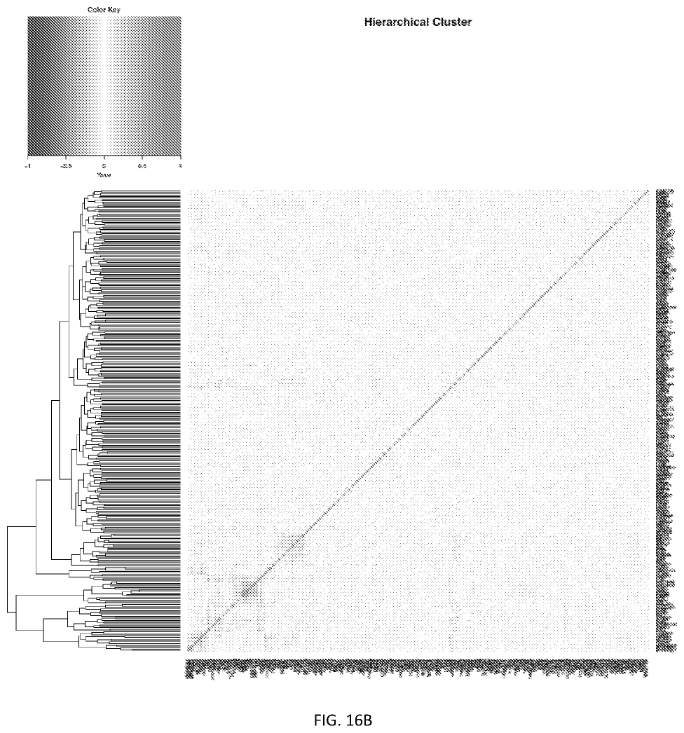

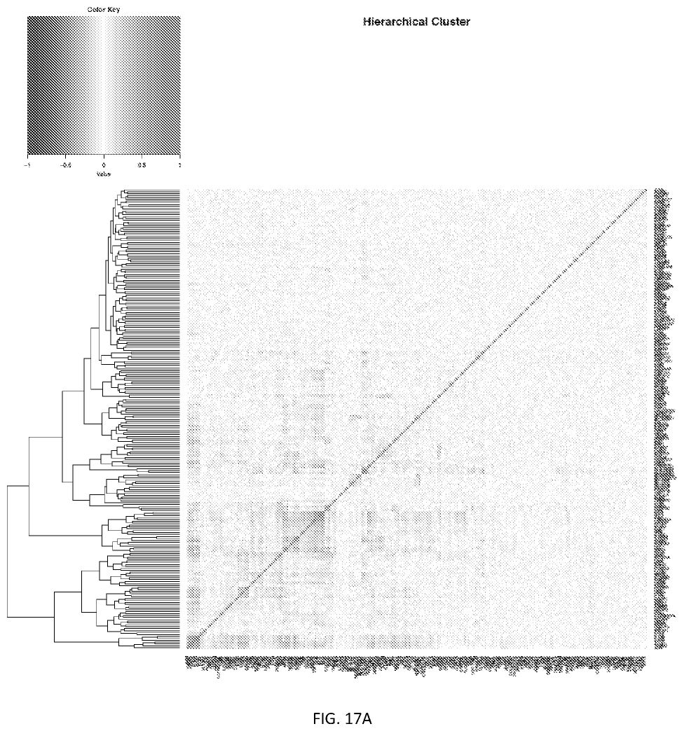

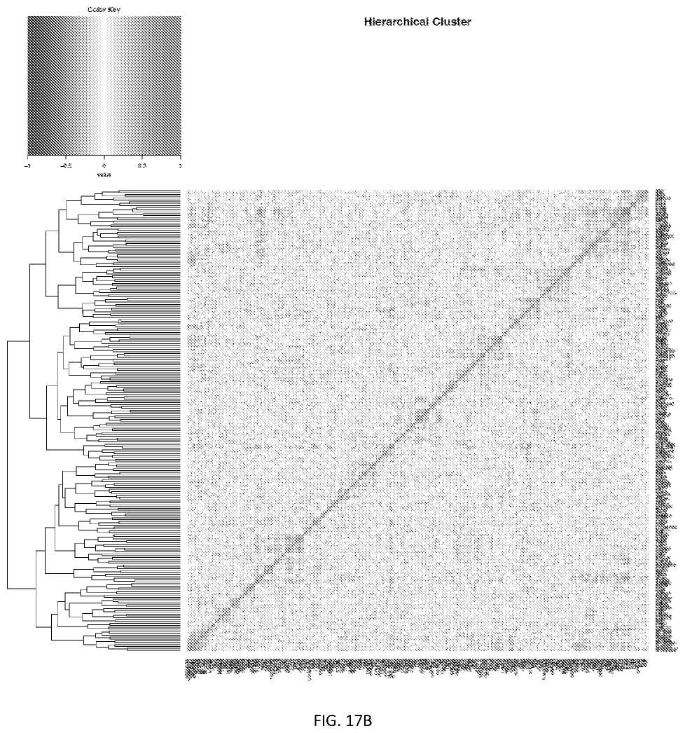

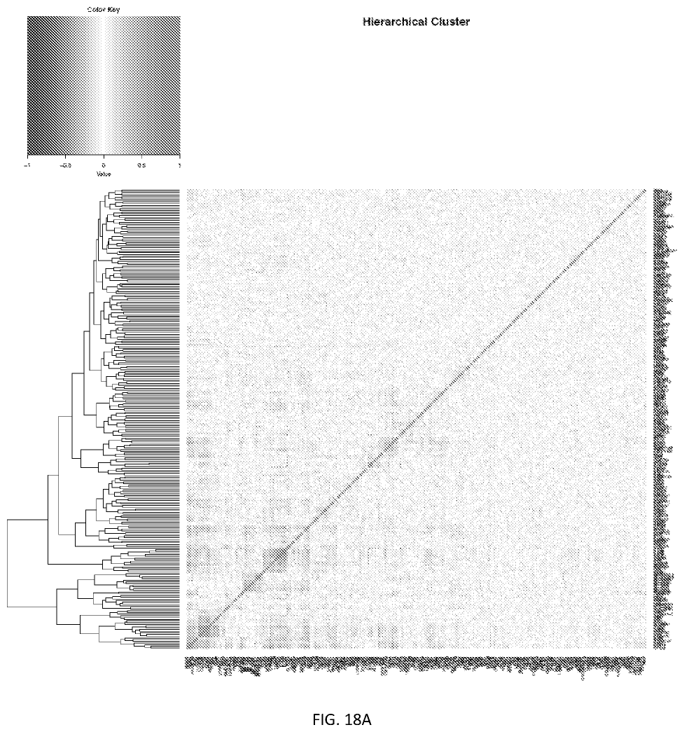

Applicants observe absolute tightening of regulation of interferon-associated genes, as these genes were included as variable genes in both sets (FIG. 17-18). However, there are very few pre-treatment macrophages, so this may be inflated in comparison.

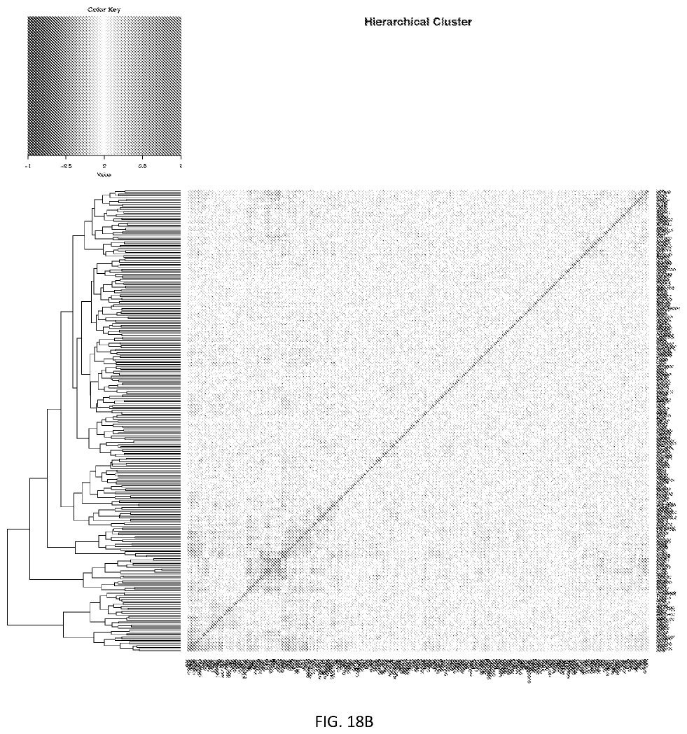

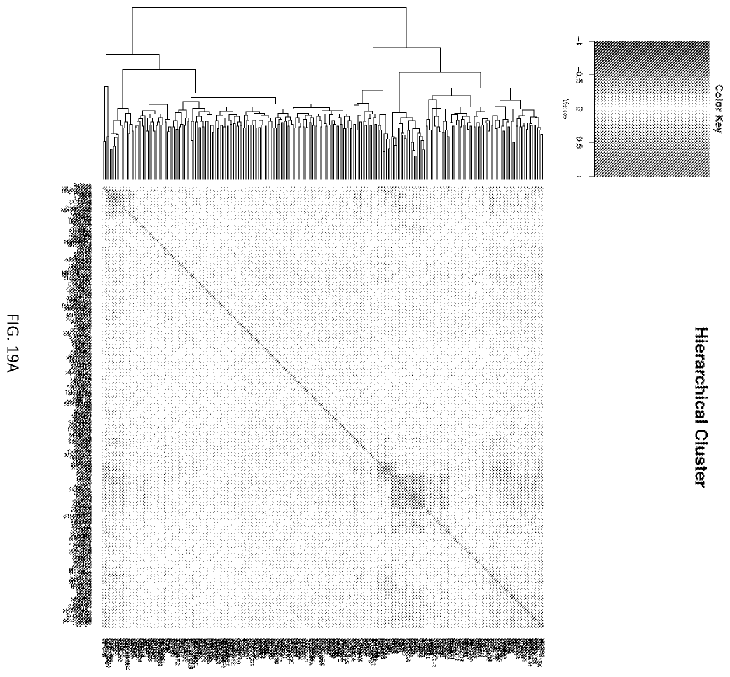

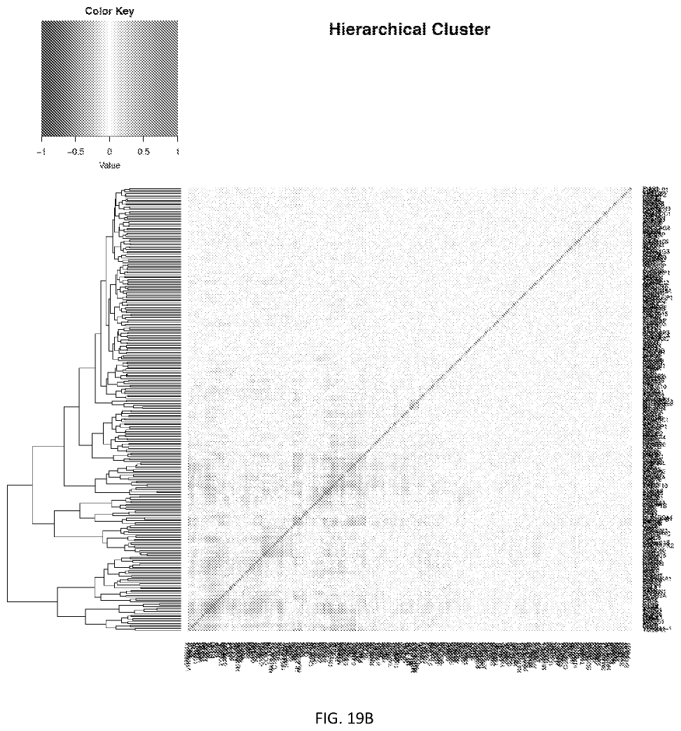

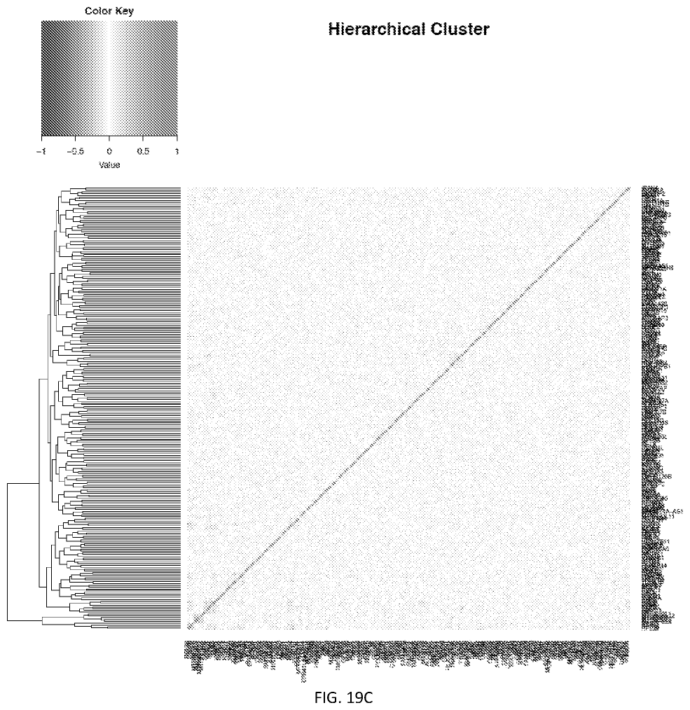

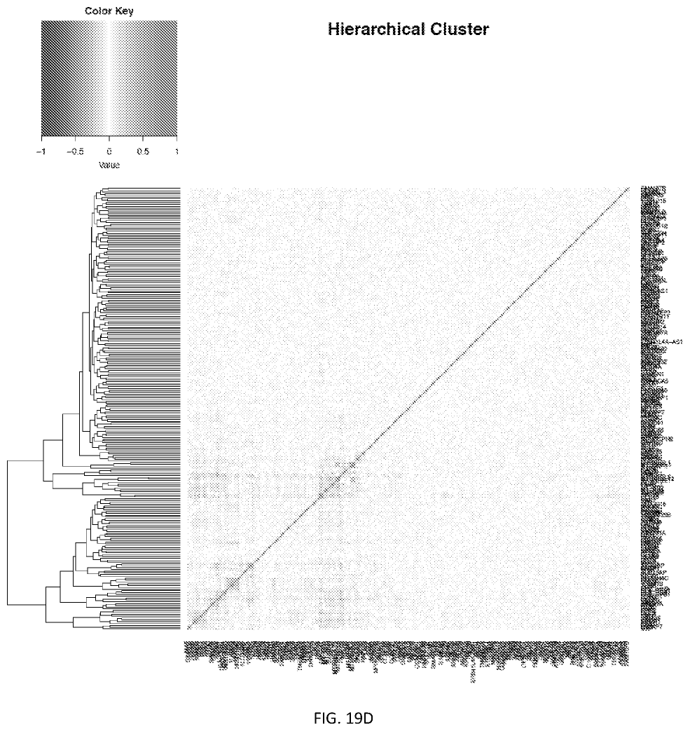

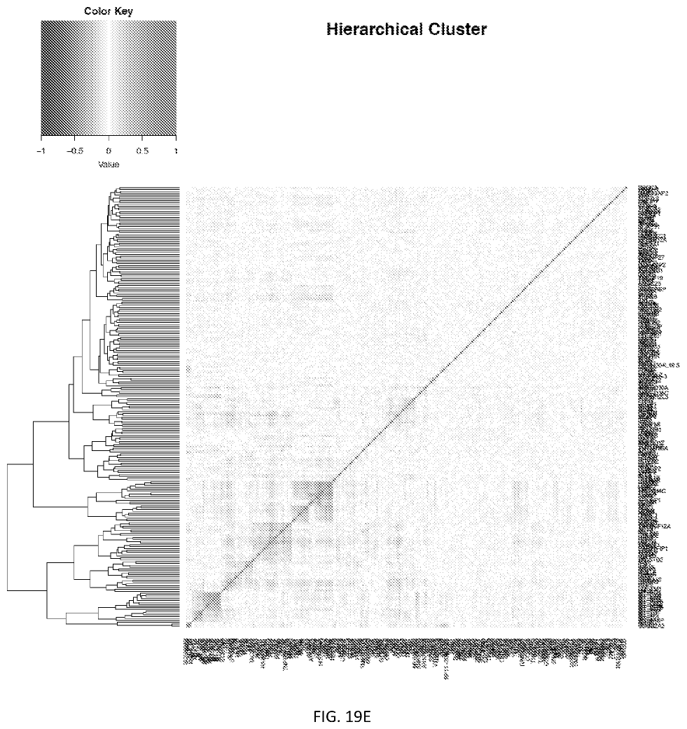

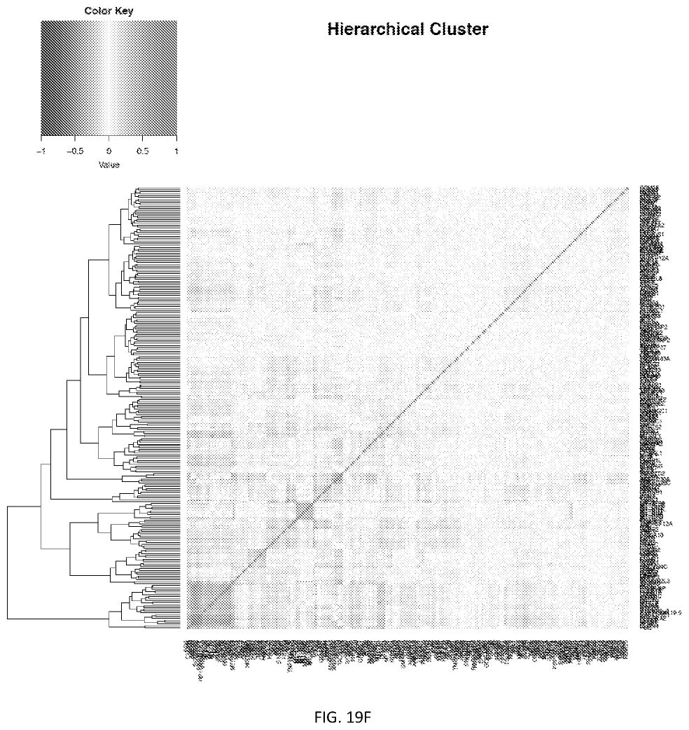

Applicants compared cells captured from cerebrospinal fluid from patients who have brain metastases but not LMD to cells captured from cerebrospinal fluid from patients who have LMD (FIG. 19). Tumor populations from LMD demonstrate increased tightening of correlation of FOSB and CLDN genes, genes involved neuroplasticity. T cell populations from LMD demonstrate increased response signaling through interferon. Macrophage cells in the patient with brain metastasis have an increased bias towards complement and other tumor-targeting phenotypes. This is especially evident considering the majority of macrophage cells from BM do not express classic TAM signature genes, as do a subpopulation of macrophages from the LMD patients. Applicants observed more convincing evidence of tightening of regulation of interferon-associated genes. This is consistent with the previous findings looking across multiple patients in reduced dimension space as well as the microenvironment of these patients as corroborated by the analysis of the T cells and tumor cells.

### Example 3—A Phase-II Clinical Trial to Examine the Efficacy of CBI in LMD

To more thoroughly understand the potential therapeutic benefits of CBI in LMD, Applicants initiated a phase-II clinical in which CSF was repeatedly sampled from breast cancer patients with LMD. Criteria for enrollment included a histologically confirmed solid malignancy and CSF cytology demonstrating LMD. Patients were treated with a ventriculoperitoneal shunt (VPS) for symptomatic management and for CSF sampling. Intravenous Pembrolizumab was administered once every 3 weeks (defined as a treatment cycle), consistent with previously reported dosing regimens. Following a treatment cycle, each patient's VPS was accessed for CSF collection, which was then profiled by Seq-Well if cellular material was available following clinical procedure (FIG. 20A). A total of 11 out of 18 enrolled patients were included for single-cell analysis, 4 of which were sampled at multiple time points across the course of treatment. All of these 11 were diagnosed with metastatic breast cancer and positive CSF cytology indicative of LMD. Given the progressive, end-stage nature of LMD, 7/11 patients were on a steroid regimen at the time of enrollment and 8/11 patients were treated with steroids while receiving pembrolizumab. Overall, 7 patients lived beyond 3 months post treatment initiation (primary endpoint for the trial), suggesting potential clinical benefit for CBIs in breast-cancer-derived LMD.

### Example 4—Characterizing the Leptomengeal Disease (LMD) Tumor Microenvironment (TME)

In light of potential safety concerns, especially given repeated sampling, only a small quantity of CSF, which has low cellular content to begin with (<50,000 cells/mL in LMD), could be collected during any single visit. To profile the cell types/states and circuits active in the LMD TME as comprehensively as possible, Applicants utilized Seq-Well, a massively-parallel scRNA-Seq platform ideally suited for low-input clinic samples such as CSF. Applicants performed Seq-Well on 19 distinct samples from 11 patients, obtaining 13,452 single cells after quality control using standard complexity filtering and doublet removal techniques.

Applicants first sought to identify the cellular constituents of LMD. Starting with a genes-by-cells expression matrix, Applicants performed dimensionality reduction using principal component analysis (PCA), identified clusters with a shared nearest neighbors (SNN) algorithm, and then visualized using uniform manifold approximation and projection (UMAP) (see, e.g., Becht et al., Evaluation of UMAP as an alternative to t-SNE for single-cell data, bioRxiv 298430; doi.org/10.1101/298430; and Becht et al., 2019, Dimensionality reduction for visualizing single-cell data using UMAP, Nature Biotechnology volume 37, pages 38-44). This revealed eleven distinct clusters comprised primarily of malignant (tumor) cells, as well as adaptive and innate immune cells (FIG. 20B). The malignant cell clusters (n=8) demonstrated strong patient-specific representation while the immune clusters (n=3) grouped by phenotype rather than by patient (FIG. 20B), as has been observed previously in other single-cell transcriptional profiling of human tumors.

To confirm that the identified “tumor” clusters were in fact malignant cells, Applicants used the single-cell expression data to infer cellular CNV profiles. In this analysis, Applicants used whole exome sequencing (WES) of the same tumor microenvironment to benchmark the single-cell windowed mean expression (WME) of tumor and non-tumor cells. Applicants observed that the median slopes of plots of tumor cells WME vs. WES from the same patient were significantly positive and higher than those of non-tumor cells. Additionally, Applicants found that such positivity was highly sensitive to matching correctly WES and WME data by patient, enabling orthogonal, patient-specific identification of malignant cell state. Thus, Applicants identified—and hereafter refer to—cells belonging to called patient-specific, non-immune clusters in the data as tumor cells.

Cell-type composition was highly variable across patients, both pre- and post-treatment. In some patients, Applicants observed several tumor cells pre-treatment (e.g., patient 14 sample 1 and patient 43 sample 2 (hereafter, P014-1 and P043-2, respectively), while in others (P037-1, P042-1), Applicants did not. Immune cells, on the other hand, were detected in almost every patient post-, but not pre-, treatment. Concordantly, in the three patients who were sampled both before and after CBI introduction, the relative abundance of immune cells increased (patients 14, 29, 43). In total, Applicants recovered considerable numbers of cells of each type pre- and post-treatment, enabling direct investigation of the impact of Pembrolizumab on the LMD TME.

### Example 5—CBI Increases T Cell Abundance and Expression of Genes Associated with Cytotoxicity and Proliferation

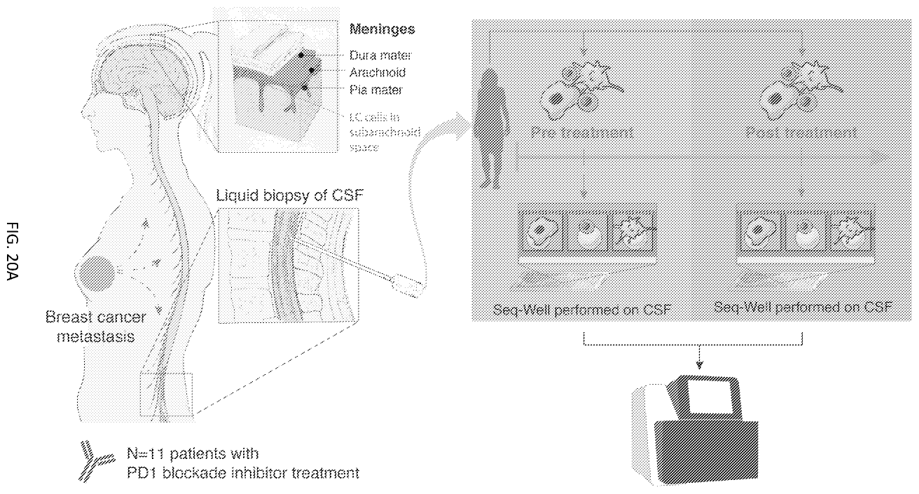

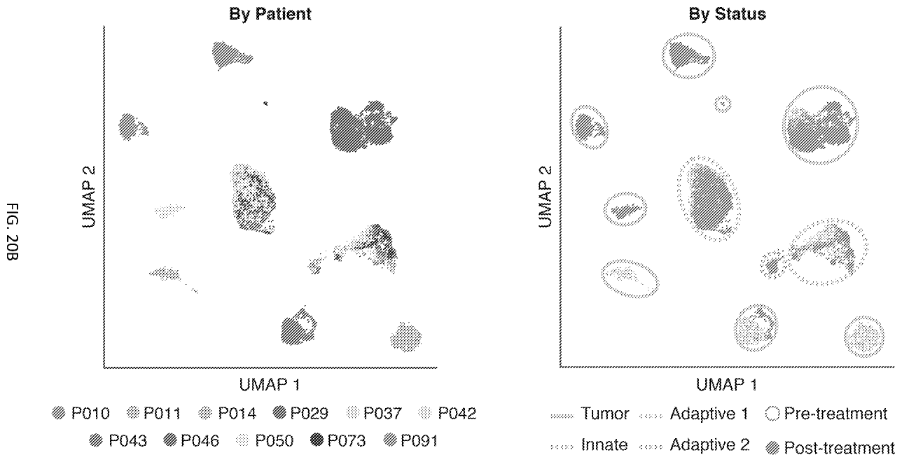

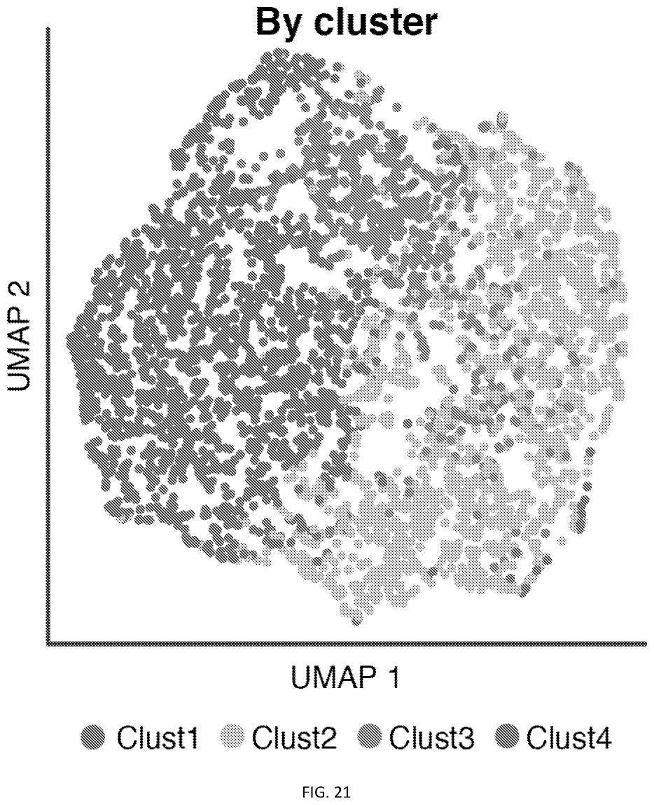

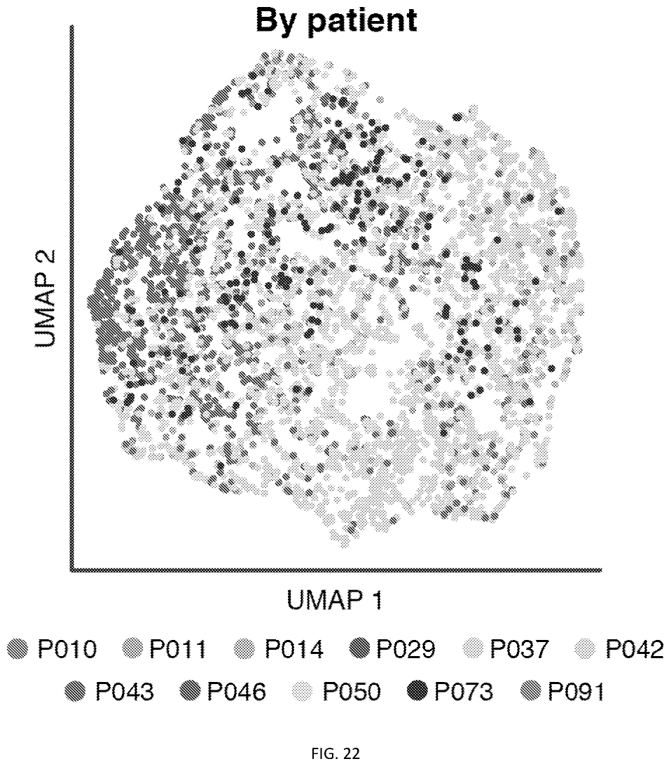

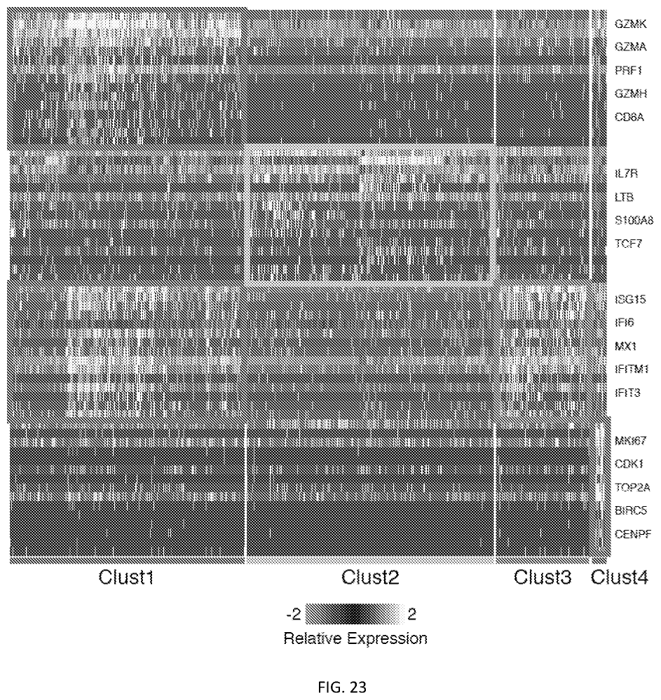

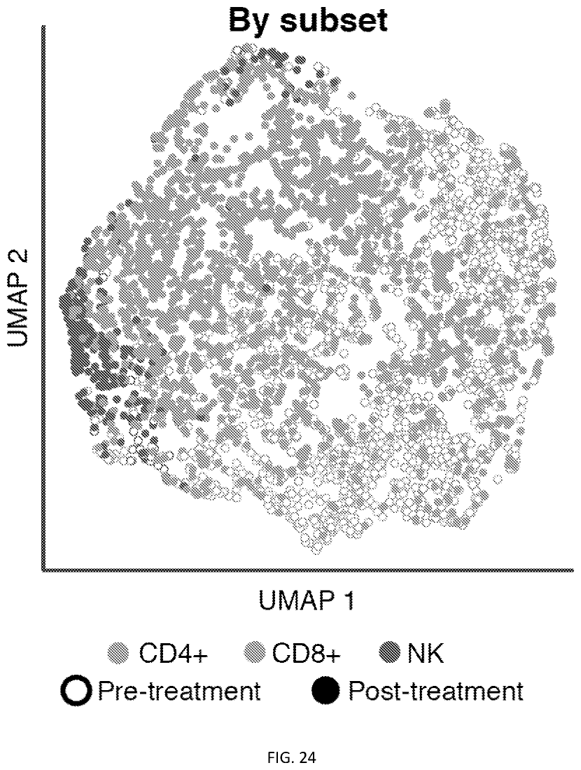

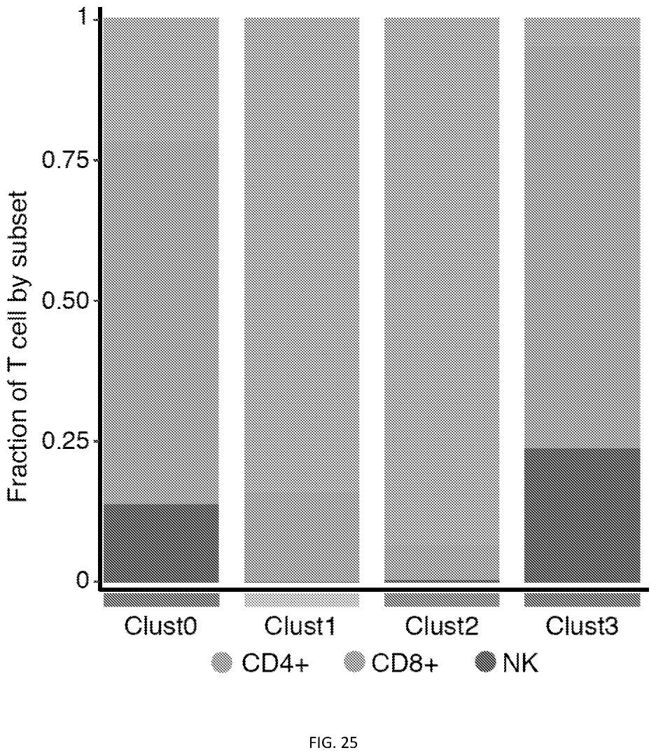

Pembrolizumab acts by binding to PD-1 expressed primarily on the surface of T cells. Thus, Applicants first sought to investigate how CBI treatment might alter the phenotypic properties of TME T cells. A brief examination of marker genes for the main two adaptive immune clusters revealed that one was enriched for canonical markers of T and NK cells (e.g., CCL5, CD3D, IL7R), and the other for those of B cell lineage (e.g., IGJ, IGKC, SLAMF7). Variable gene selection, dimensionality reduction, and clustering, as above, partitioned the former into four subgroups of interferon-/cytokine-enriched, resting/memory, interferon-enriched but not cytokine-enriched, and proliferating cells, (FIGS. 21-23) but did not immediately resolve classic adaptive immune subsets, such as CD4+ or CD8+ T cells. Thus, Applicants utilized iterative subclustering to identify CD4+ and CD8+ T cells, as well as NK cells (FIG. 24) (which, based on gene-expression similarity, typically cluster with adaptive immune cells in scRNA-Seq data). Notably, the interferon-/cytokine-enriched and proliferating subsets were primarily comprised of CD8+ T cells as well as NK cells, while the resting/memory and interferon-enriched non-cytokine subset was predominantly comprised of CD4+ T cells (FIG. 25).

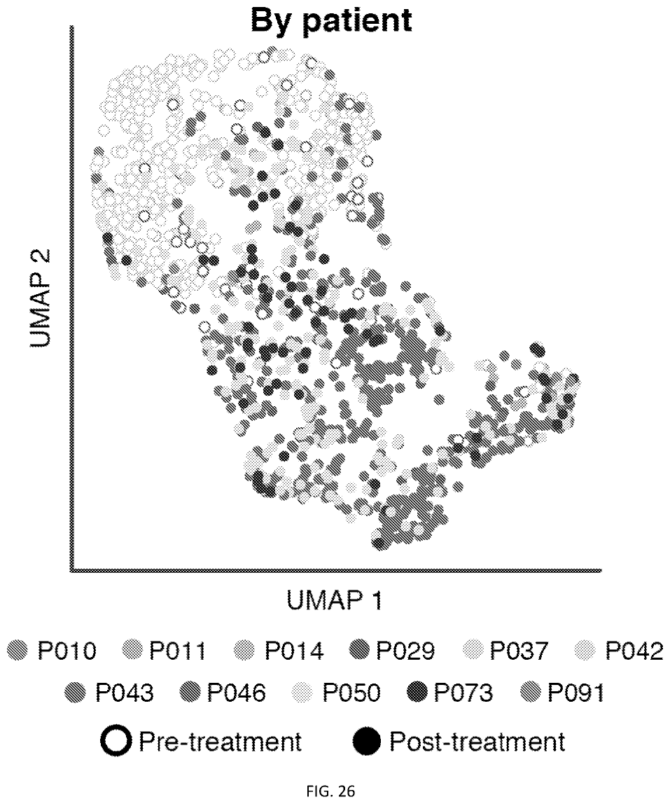

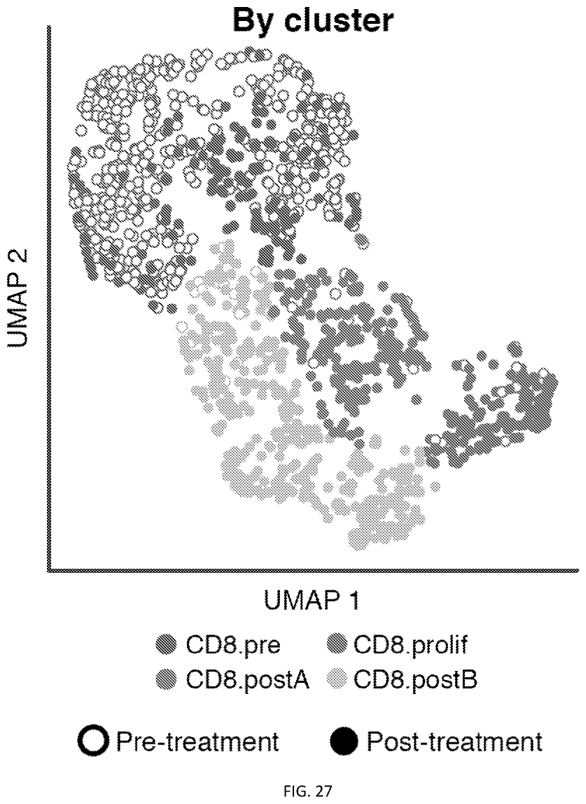

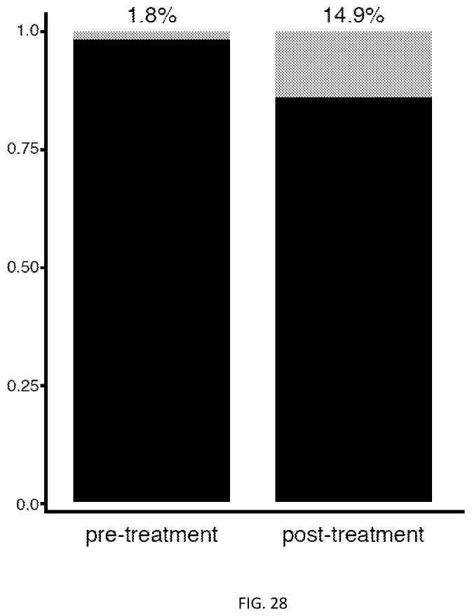

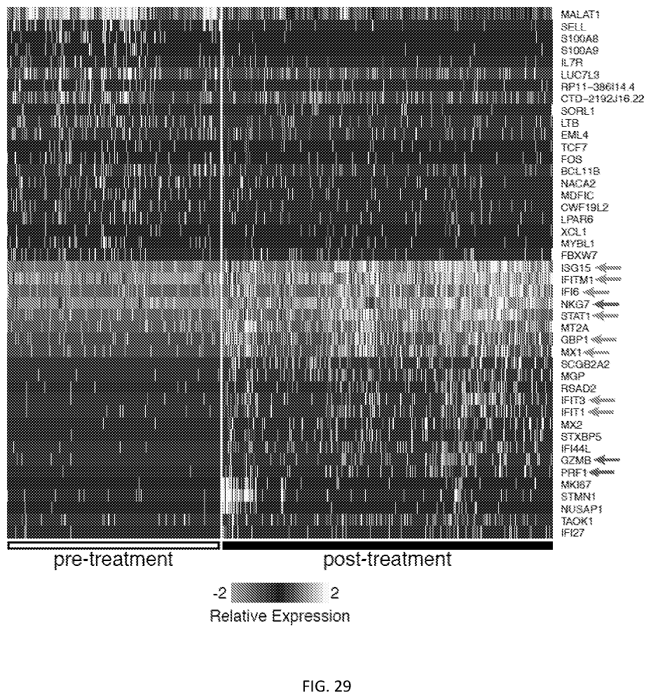

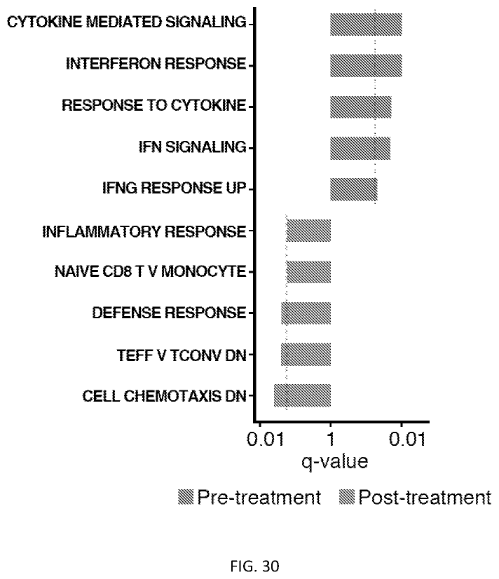

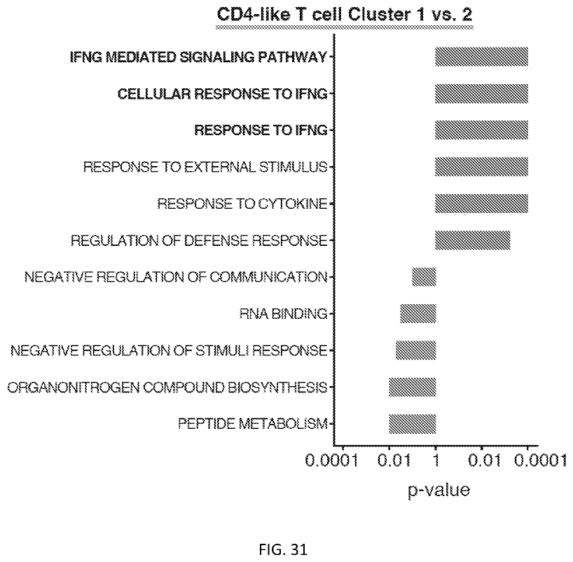

Applicants next combined all CD8+ T cells across all patients to more closely examine how their behavior was affected by treatment (FIG. 26). Further analysis of the CD8+T cells revealed 4 distinct clusters: one dominated by pre-treatment cells (CD8.pre), two by post-treatment cells (CD8.post1 & CD8.post2), and one which expressed high-levels of genes associated with proliferation (CD8.prolif) (FIG. 27). CD8.prolif was also primarily composed of post-treatment cells, and it comprised a larger fraction of the post-treatment cells than pre-treatment (14.8% vs. 1.8%) (FIG. 28). A comparison of the treated versus untreated CD8+ T cell clusters identified a set of differentially expressed genes containing canonical markers of cytotoxicity (NKG7, PRFI, GZMB) and interferon-response (ISG1S, STAT1, MX1) (FIG. 29), as well as overall enrichments of leukocyte activation, interferon-γ signaling and response, and cytokine signaling pathways (FIG. 30). Genes upregulated in untreated CD8+ cells, meanwhile, included IL7R, S100A4, BCL2, which have previously been associated with memory/effector T cell state (Tables). Applicants observed a similar drug-induced bifurcation in CD4+ T cells, with higher interferon-mediated signaling and interferon response (FIG. 31), post-treatment. These data suggest the possibility that Pembrolizumab might recover cytotoxicity, interferon response, and proliferation in CD8+ T cells.

### Example 6—T Cell Exhaustion is Heterogeneously Affected by CBI Introduction

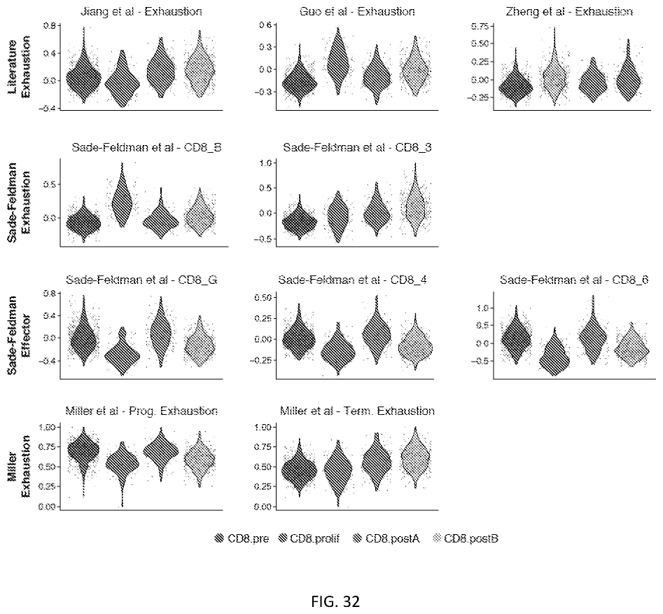

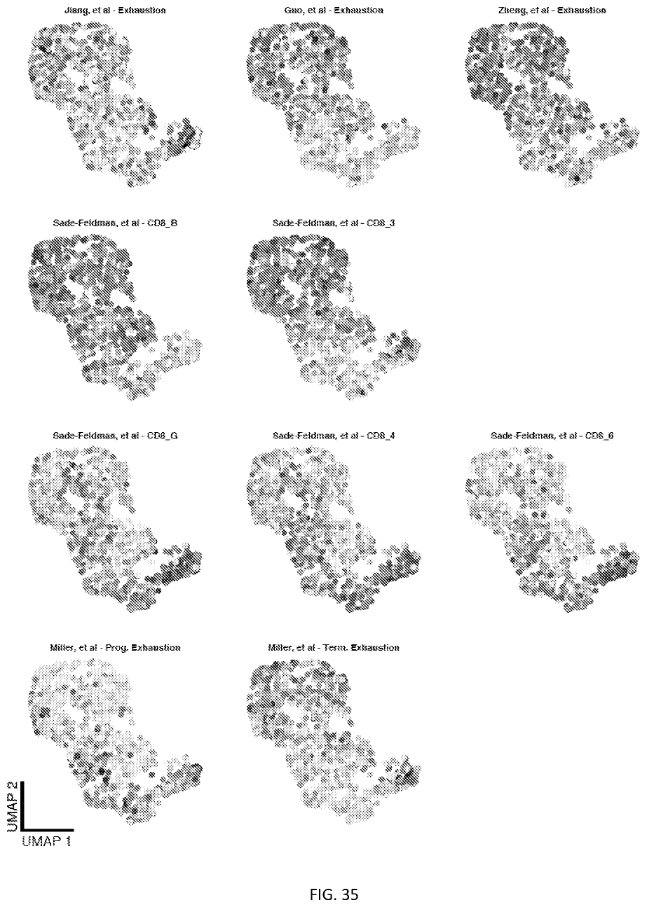

To more explicitly examine potential mechanisms for recovering cytotoxic or proliferative phenotypes, Applicants further investigated CD8+ T cell exhaustion by scoring the cells against a series of curated signatures (Jiang et al, Miller et al, Sade Feldman et al, Guo et al, Zheng et al) (FIGS. 32 and 35). The results suggest that post-treatment CD8+ T cell subsets are more exhausted than pre-treatment cells (FIG. 32). Importantly, the degree of exhaustion varies significantly between subsets, with CD8.post1 exhibiting a much more exhausted profile than CD8.post2 (FIG. 32)—Applicants hereafter refer to CD8.post1 and CD8.post2 as CD8.exh^{hi} and CD8.exh^{lo} respectively. Interestingly, CD8.exh^{hi} also exhibits significantly higher levels of cytotoxic gene expression than both CD8.pre and CD8.exh^{lo} subpopulations (e.g., upregulation of PRFI, NKG7, and GZMK expression), as well as increased interferon response.

Recent work has demonstrated that T cell exhaustion can manifest as multiple heterogeneous states. In particular, exhausted CD8+ T cells have been assigned to (at least) two mutually-exclusive groups: one with high levels of stemness, reduced cytotoxicity, and increased polyfunctionality, and the other with increased cytotoxicity, higher levels of classical exhaustion markers (TIM3, CD39), and reduced cell survival (Wherry and Kurachi, 2015, Nature reviews Immunology 15, 486-499). Applicants sought to investigate whether variation in the exhausted CD8+ T cells was concordant with this classification, and how this association changes with continuous Pembrolizumab treatment.

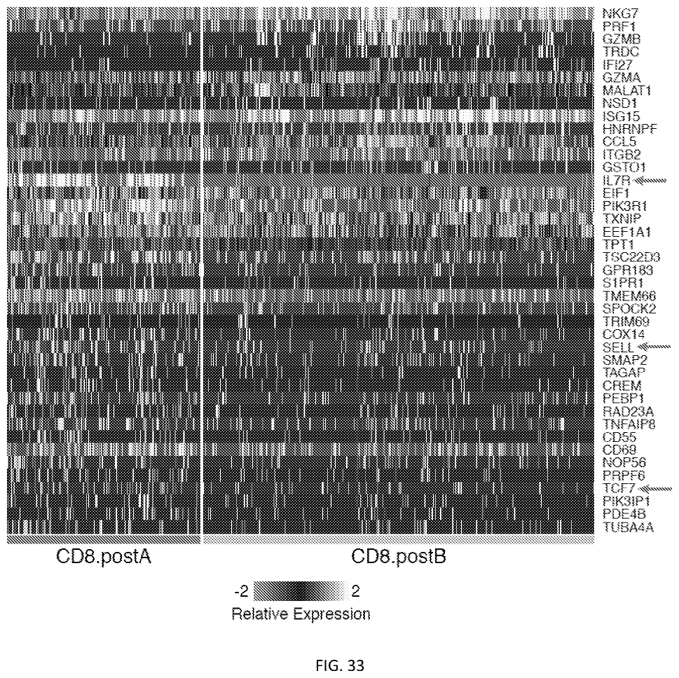

Thus, Applicants scored the CD8+ T cells according to recent work describing this heterogeneity as two distinct phenotypes—progenitor and terminally exhausted CD8+ T cells (Miller et al). Applicants observe high concordance between the clusters and these states: CD8.exh^{lo} cells more closely resemble “progenitor exhausted” T cells, while CD8.exh^{hi} cells resemble “terminally exhausted” T cells (FIG. 32). Further, Applicants investigated recently described distinction between CD8+ T cell exhaustion and effector phenotypes in CBI treatment. Here, Applicants find that CD8.exh^{lo} cells exhibit increased effector function relative to CD8.exh^{hi} (FIG. 32), as well as higher expression of genes including IL7R and SELL, which are more associated with effector/memory phenotype. Additionally, TCF7, a marker for positive patient prognosis with CBI investigated in these and other works (Sade-Feldman et al., Cell. 2018 Nov. 1; 175(4):998-1013.e20; and Kurtulus et al., Immunity. 2019 Jan. 15; 50(1):181-194.e6) is also upregulated in CD8.exh^{lo} (FIG. 33).

Among the top differentially expressed genes in CD8.exh^{hi} are TRDC and TRGCI, the glycoproteins of the T cell repertoire (TCR) specific to γ-δ T cells. Applicants identify the small cluster of CD8.exh^{hi} cells jointly upregulating γ and δ repertoire genes, and confirm that they are likely not contaminating NK cells (which also express γ-δ TCR genes), demonstrating low expression of CD16, high expression of CD3, and constant rate of detection of variable chain genes in this group of cells (27.6% in γ-δ T cells, 17.5% in other CD8+ T cells). γ-δ T cells are a rare population of T cells commonly found in mucosal niches with largely incomplete characterization of antigenic properties to date. However, recent work has demonstrated that checkpoint blockade inhibition can lead to increased clonal proliferation of this phenotype. It is possible, then, that γ-δ T cells achieve a terminally exhausted phenotype and participate in the interferon-induced cytotoxic T cell rejuvenation demonstrated in LMD.

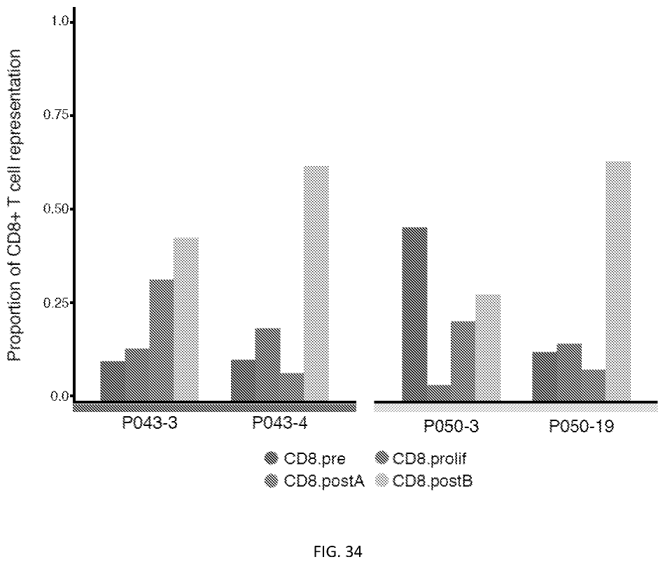

Due to previously described difficulties in longitudinal sampling, the temporal dynamics of CD8.exh^{hi} and CD8.exh^{lo} states have yet to be deeply profiled from the same cellular reservoir in human patients. The patient cohort includes two patients for which multiple post-treatment draws are available (FIG. 34), and so Applicants sought to investigate these two states over the course of Pembrolizumab treatment. Delineating time points by cluster reveals that in the earliest post-treatment time point in each patient (P043-3 and P050-3 respectively) approximately 30% and 20% of CD8+ cells belong to the CD8.exh^{lo} cluster. At later time points P043-4 and P050-19, representation decreases to approximately 8%, while representation of CD8.exh^{hi} cluster rises from 43% and 28% to over 60% in both patients. This indicates the CD8.exh^{lo} phenotype may be more associated with earlier Pembrolizumab response than later, and the terminally exhausted CD8.exh^{hi} predominates especially later. In fact, recent work characterizing T cell exhaustion patterns following CBI suggest that PD1-blockade transitions stem-like exhausted CD8+ T cells towards highly cytotoxic, interferon-producing terminally exhausted CD8+ T cells. The data lend further credence to this model of exhausted CD8+ T cell reactivation, and—crucially—Applicants describe congruent temporal shifts here in the exact same TME over time.

### Example 7—Innate Immune Cells Exhibit Pro- and Anti-inflammatory Responses to CBI

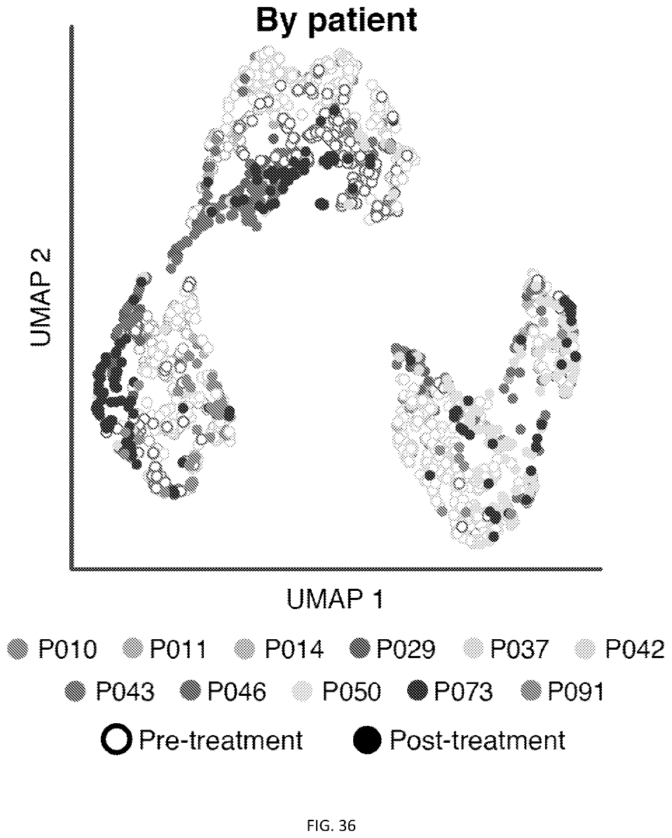

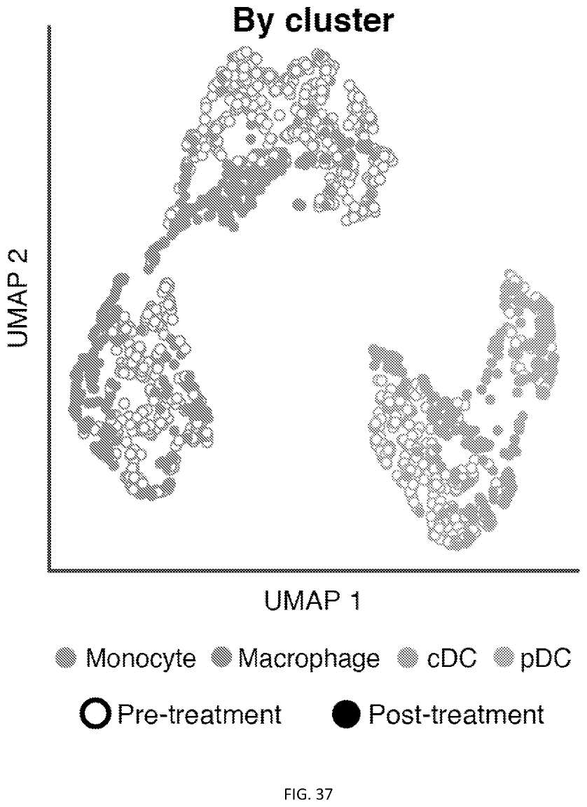

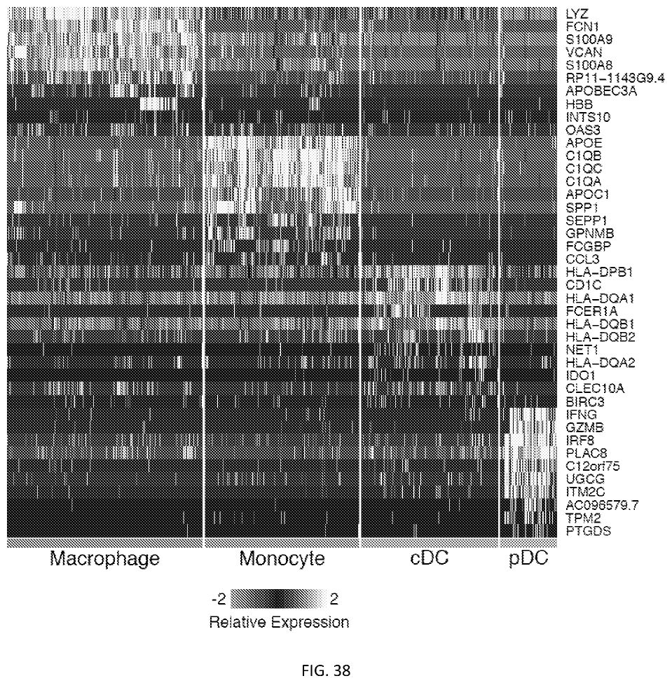

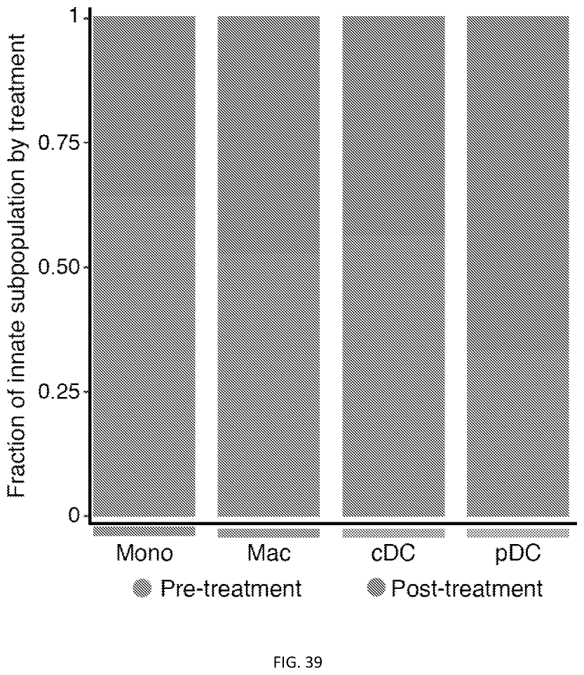

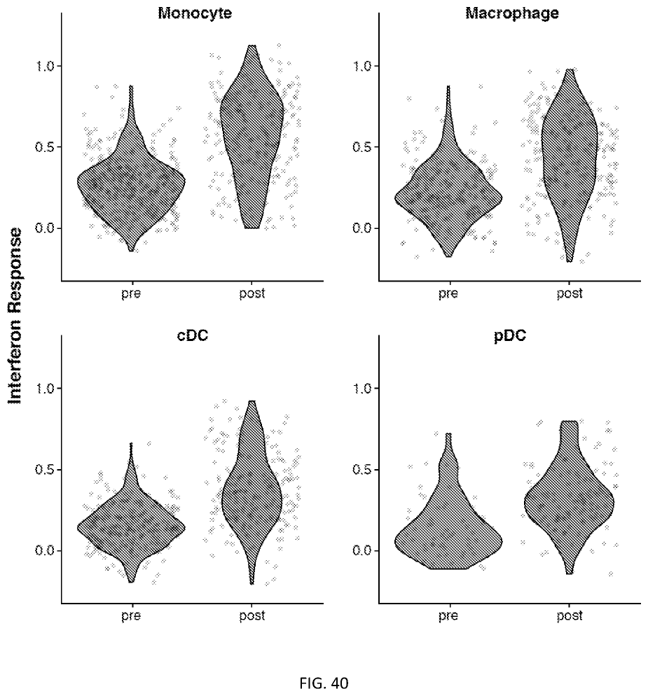

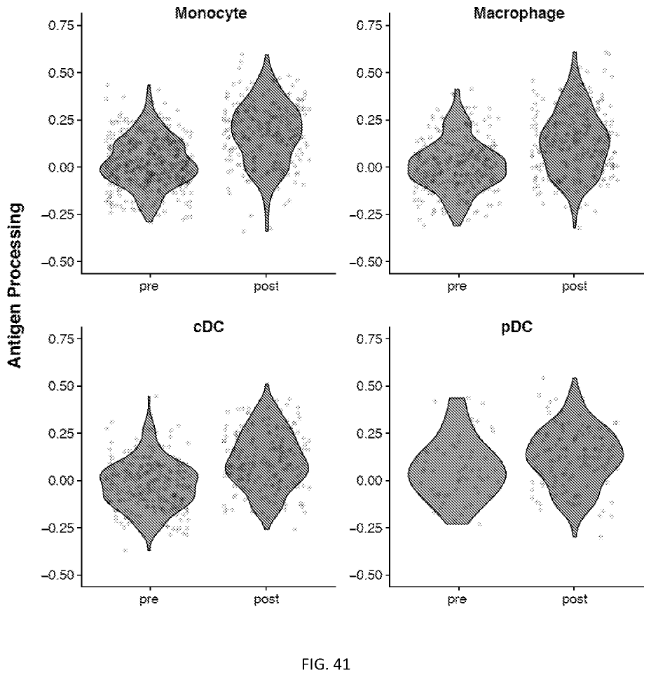

Applicants next investigated the effect of Pembrolizumab treatment on the innate immune cells of the LMD microenvironment, seeking to contextualize differences in activity observed in T cells within the broader immune response. Unsupervised analysis, performed as before, identified inherent subpopulations consistent with canonical phenotypes—monocytes, macrophages, classical dendritic cells (cDCs) and plasmacytoid-derived dendritic cells (pDCs) (FIGS. 36-38). pDCs are the only innate phenotype comprised of more post-treatment cells than pre-treatment cells. Since pDCs have been shown to produce large quantities of interferon in response to antigen (FIG. 39), they may potentially drive, in part, the observed interferon response in the TME. Differential expression between pre- and post-treatment cells within each subset also reveals that post-treatment cells maintain increased levels of interferon response across all innate phenotypes. (FIG. 40). In addition, Applicants find that all innate subpopulations exhibit improved antigen-processing post-treatment, likely resulting from STAT1 activation and consequential signaling (FIG. 41).

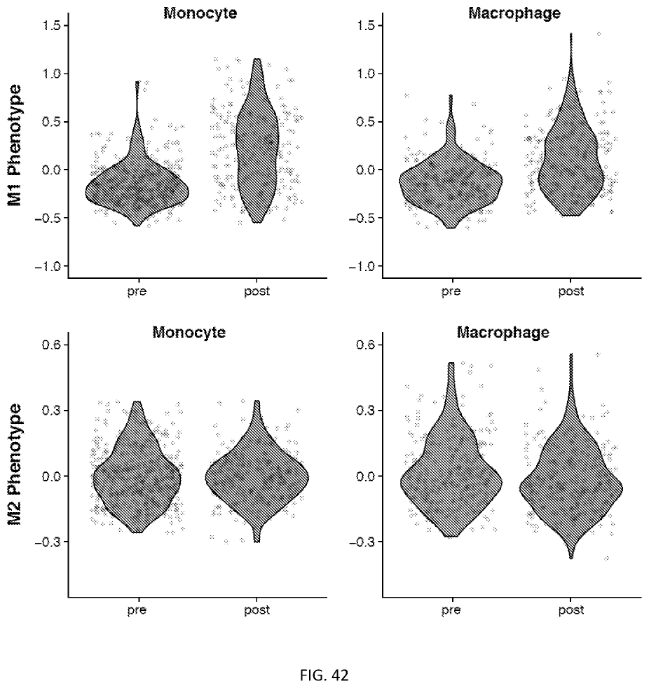

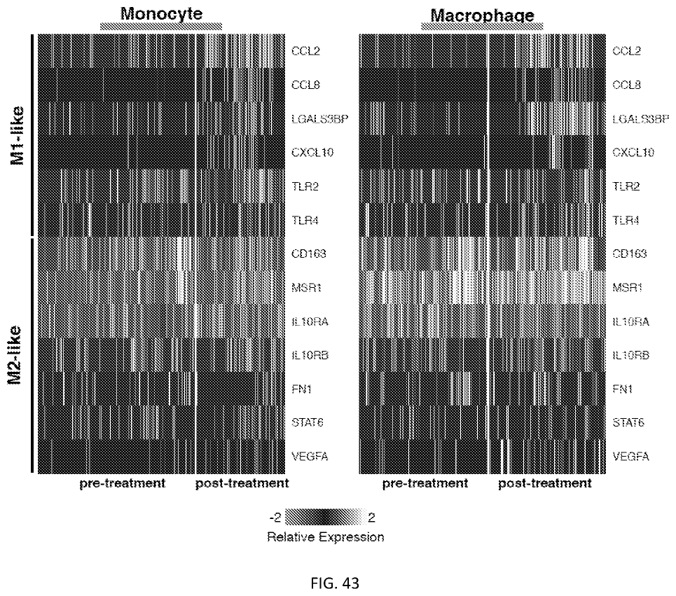

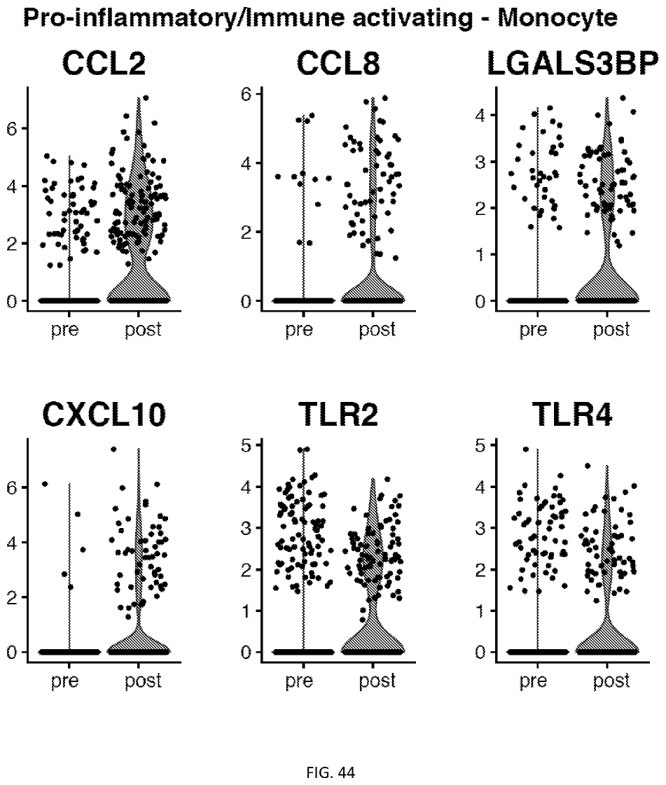

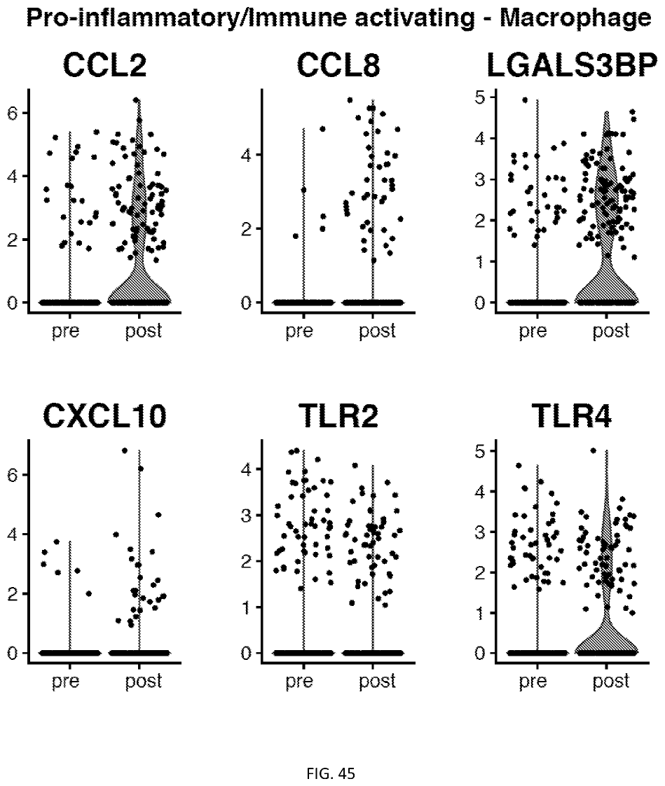

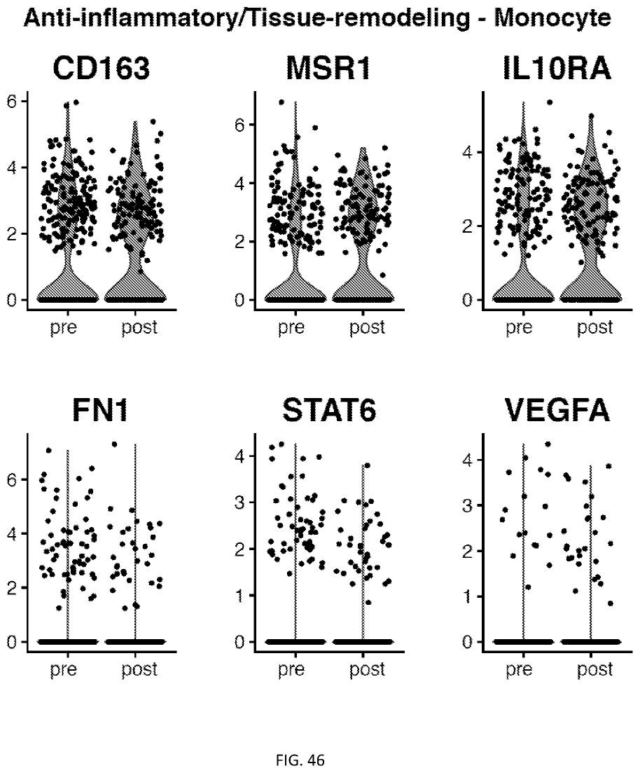

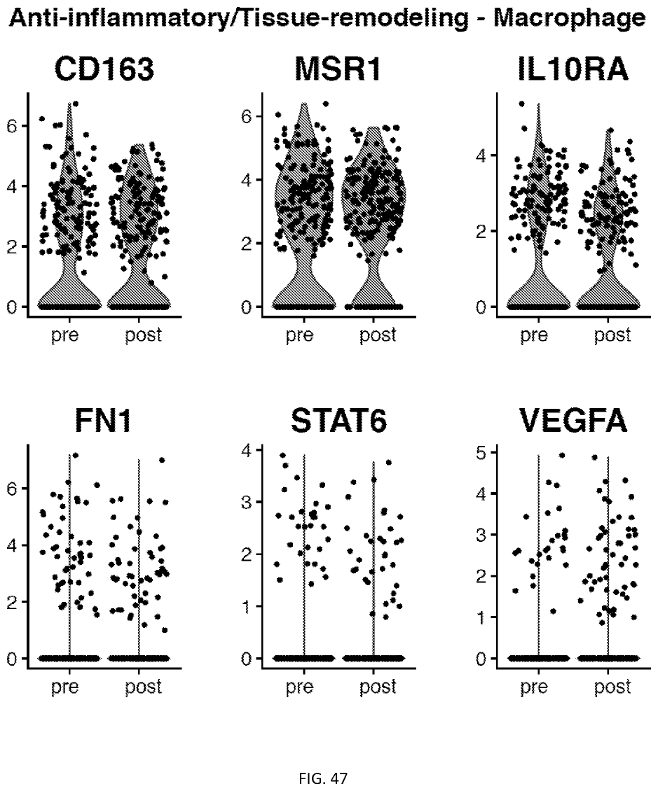

Applicants next investigated further the monocyte and macrophage clusters, specifically examining their tumor-associated polarization following Pembrolizumab introduction. Using previously curated gene sets of tumor-associated macrophage (TAM) phenotype (Jiang et al), Applicants found that, post-treatment, monocytes and macrophages exhibit significantly higher expression of signatures associated with M1-like phenotype than pre-treatment cells (FIGS. 42-43). In particular, Applicants find upregulation of genes such as CCL2, CCL8, and LGALS3BP, all of which drive cytotoxicity, defense response, and recruitment of immune cells to the tumor-microenvironment, as well as CXCL10 and TLRs which have classically been associated with M1-like behavior (FIGS. 44-45). Conversely, a M2-like signature and expression of genes classically associated with M2 phenotype—including CD163, MSR1, IL10R—maintain consistent levels across treatment in both monocytes and macrophages (FIGS. 42 and 46-47). Increased M1-like and constant M2-like functionality, as Applicants observe, suggests that while the introduction of Pembrolizumab may induce some pro-inflammatory behavior from increased interferon expression, anti-inflammatory programs can continue to persist.

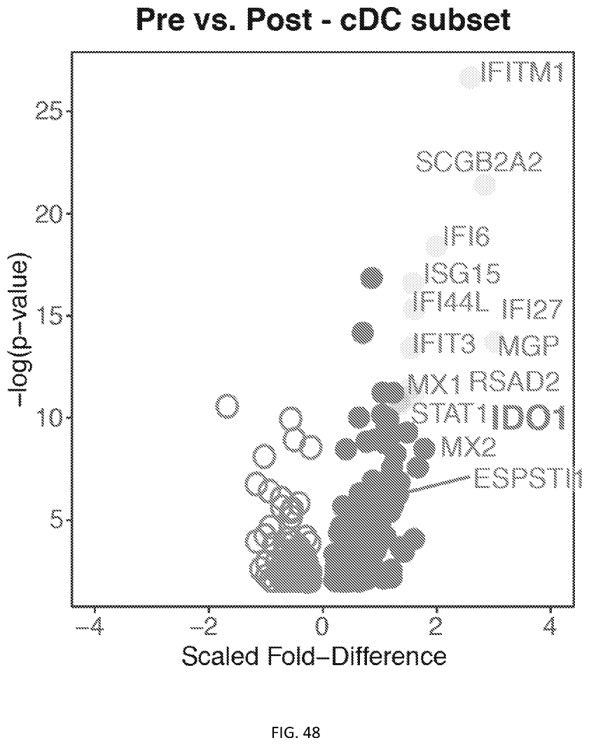

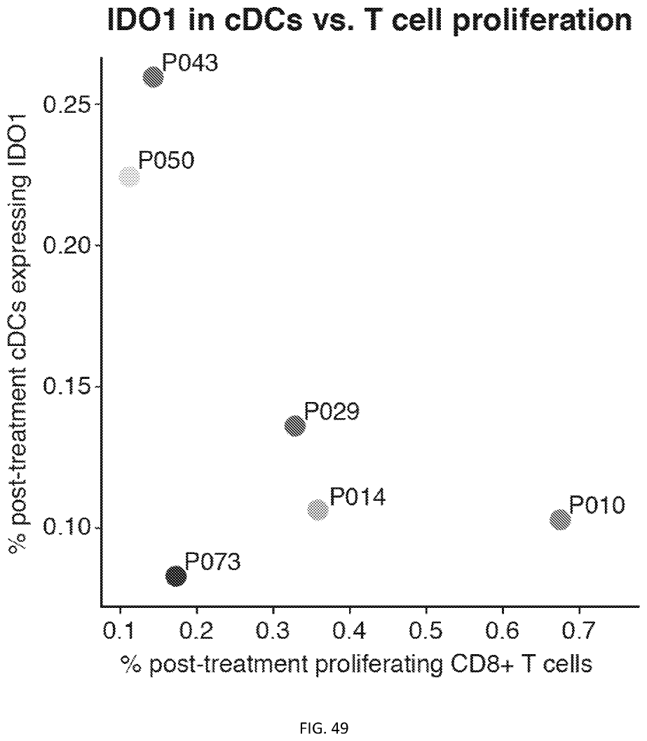

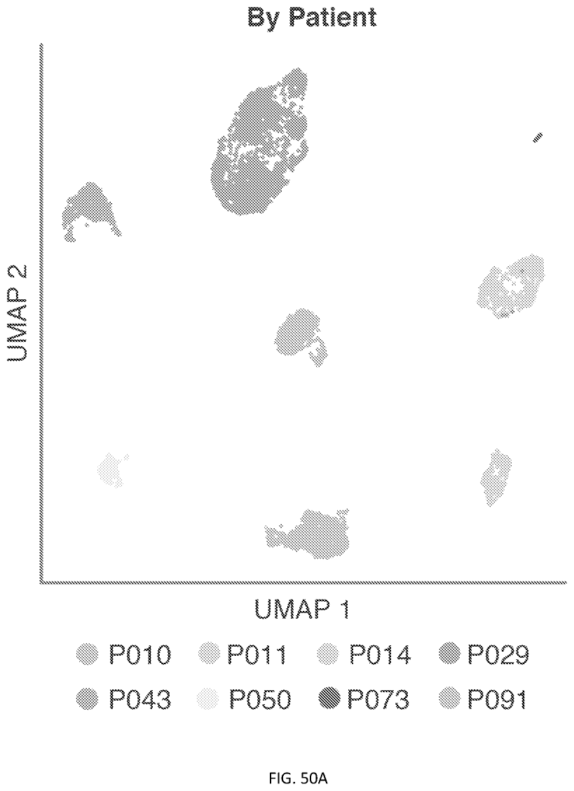

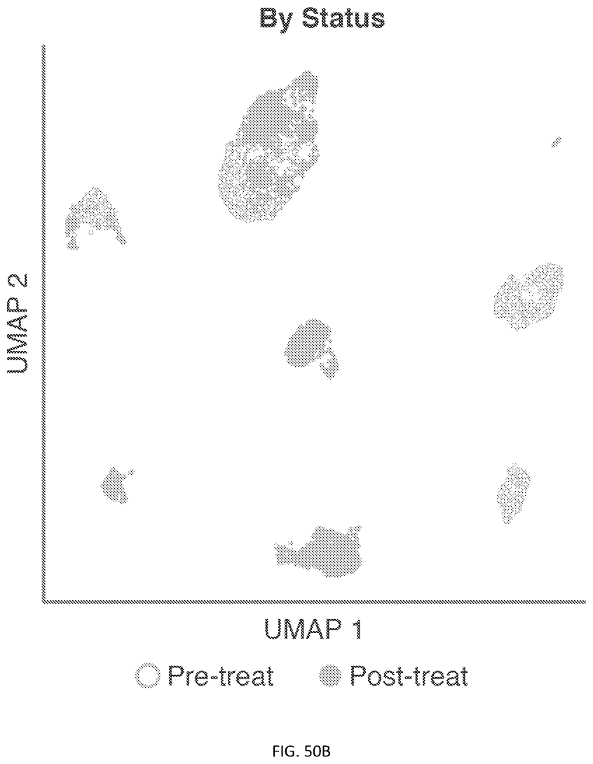

Interferon-γ in particular can drive anti-inflammatory resistance to immunotherapy, through the production of factors such as IDO1, which is frequently cited as a resistance mechanism of CBI. In the data, Applicants find that the classical dendritic cell (cDC) population upregulates interferon response genes post-treatment, as well as genes including IDO1, EPST11, and MGP, which negatively regulate immune response (FIG. 48). IDO1 production, in particular, is stimulated by interferon-γ induced JAK/STAT signaling in the tumor-immune microenvironment, and depletes tryptophan from the extracellular matrix, which is required by T cells to proliferate. This post-treatment shift in behavior of cDCs, profiled in the same LMD microenvironment, suggests the possibility that innate immune mechanisms of CBI resistance may become more active following interferon-mediated response to Pembrolizumab, complicating clinical benefit in these patients.

### Example 8—Tumor Characterization

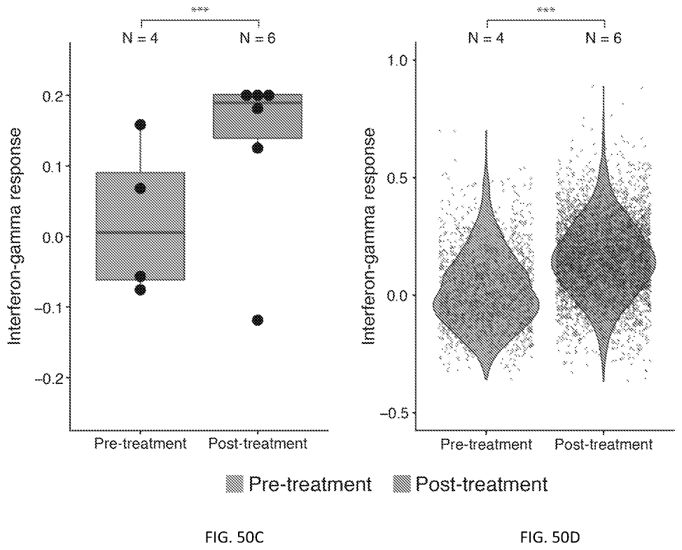

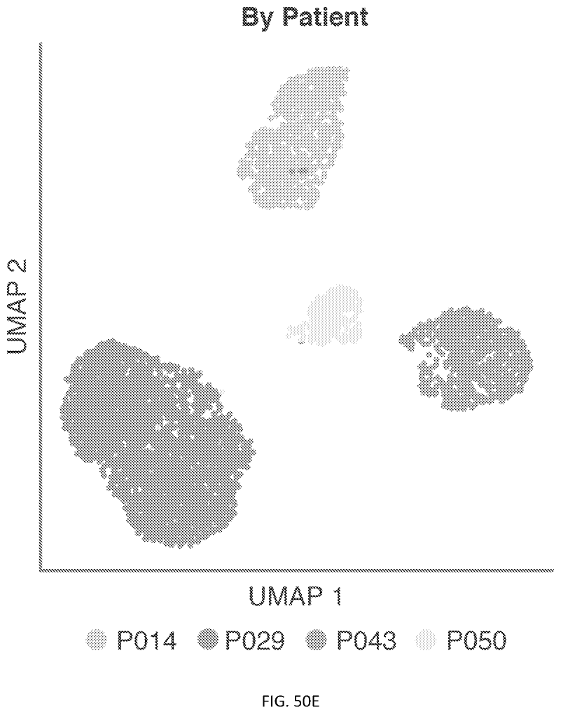

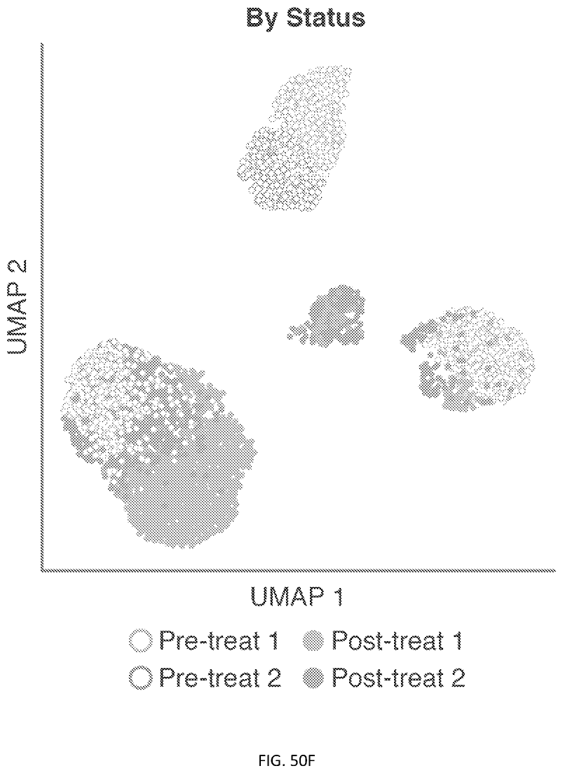

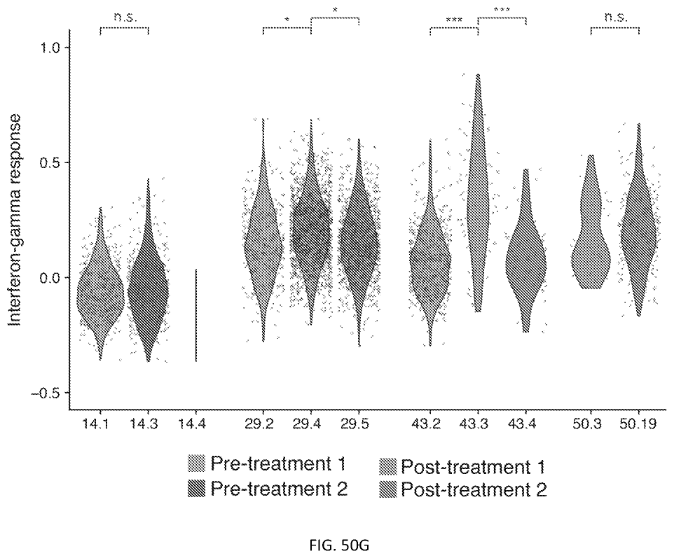

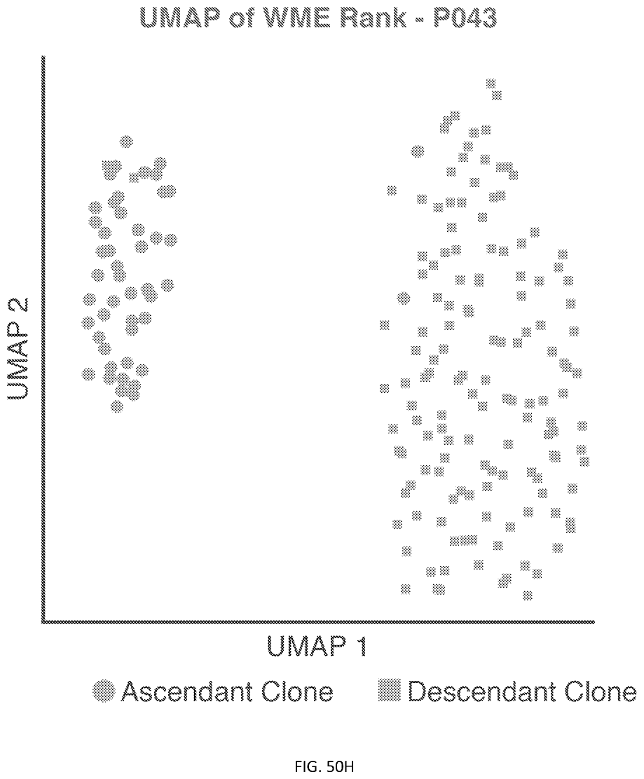

Applicants next investigated how shifts in the phenotypes of LMD TME immune cells might tumor cells in each of the patients. Subsetting and projecting all tumor cell transcriptomes in the same UMAP space reveals that the primary source of variation among tumor cells is patient-specific features (FIG. 50A-B). To mock a conventional analysis, Applicants compared pre- and post-treated tumor populations, ignoring patient labels, and found that both per-patient average and combined single-cell scores for interferon-γ response and antigen processing are upregulated post-treatment (FIG. 50C-D). This perceived difference could suggest that tumor cells post-treatment are susceptible to a more immune active microenvironment, which would be consistent with previous studies of Pembrolizumab in patient cohorts.

Importantly, this study features a subcohort of four patients with matched sampling of the same TME over time. This uniquely enables confirmation of observed tumor cell responses across patients, absent confounding patient-to-patient variability. Thus, Applicants similarly projected each of these patients' tumor cell transcriptomes with UMAP (FIG. 50E-F), and observe shifts according to their treatment trajectory. Additionally, Applicants investigated the interferon-response and antigen processing scores in each patient over time, and observed that individual patients exhibited significantly more variable baseline and post-immunotherapy responses than cross-patient comparisons might suggest (FIG. 50G).

For example, P029 and P043 exhibit statistically significant upregulation of interferon response immediately following treatment with Pembrolizumab, consistent with the general result across patients. However, the extent of response differs between the two patients, as P043 exhibits a much greater difference between its first post-treatment and pre-treatment time points than P029 (back up with effect size). In contrast, Applicants were unable to detect response in P014, as the sole post-treatment time point does not retain enough tumor cells, suggesting that the optimal window for observing tumor cell interferon response in this patient was not measured. Thus, the characterization of specific observations, especially post-treatment, in patients depends on the timing of the post-treatment measurement. Furthermore, Applicants find that different post-treatment measurements exhibit significant variability in response within the same patient. Illustratively, following the initial increase in interferon-γ response, both P029 and P043 exhibit a decrease back to appreciable (P043) or even lower (P029) levels compared to pre-treatment.

### Example 9—A Case Study of Potential Adaptive Selection Following Pembrolizumab Treatment

Applicants next sought to further explore the particularly variable interferon-response trajectory observed in P043. One possible mechanism that could help explain this dynamic behavior is selection and expansion of subclones that are less sensitive to treatment. To assess the presence of subclones, Applicants employed dimensionality reduction in ranked windowed mean expression (WME) space using UMAP as before, focusing on high-complexity cells (FIG. 50H). In P043, Applicants observe well-defined clusters in WME, concordant with gene expression clustering. One of these clusters is enriched for post-treatment cells and the other for pre-treatment; Applicants label these the ascending and descending clusters respectively. Applicants note that in another individual, P029, Applicants see substantial expressional heterogeneity, but none in ranked WME space. This expressional heterogeneity is well explained by cycling status in P029, indicating that intratumoral heterogeneity may have genetic and/or functional causes (Tirosh et al., Nature. 2016 Nov. 10; 539(7628):309-313).

Applicants plotted the WME rank for each single cell in P043 ordered by chromosome as previously reported to perform inferred CNV profiling (see, e.g., Tirosh et al., Science. 2016 Apr. 8; 352(6282):189-96) (FIG. 50I). Hierarchical clustering on this matrix identified two clusters, which are concordant with the ascending and descending clusters described previously. Applicants calculated the mutual information between these two clones in WME space, and found specific regions with the largest change in mutual information. The relationship between ascendant and descendant cluster values at these regions is concordant with the mean window tCR in WES space, suggesting that shifts in WES profiles over time could be related to the abundance of each cluster across time, and that these clusters may be subclones with distinct genetic—and subsequent transcriptomic—features.

Applicants wanted to further examine if the change in proportion of these clusters was associated with the onset Pembrolizumab treatment. Applicants perform differential expression analysis between the ranked WME clusters in P043, both in aggregate and stratified by time point so as to control for batch effects. Applicants observe that many differentially expressed genes are associated with interferon-γ response, and Applicants plot interferon response scores for each cluster at the three available time points (FIG. 50J). These data show that the descendent cluster exhibits higher interferon response at the time point of highest overall activity (seen in FIG. 50G). In contrast, the ascendant cluster (which is originally underrepresented before treatment) exhibits consistently lower interferon response across this trajectory, eventually overtaking the descendant cluster in representation at the latest time point. These data suggest that the change in proportion in these clusters may be explained by adaptive selection unique to a pembrolizumab-induced immune response.

### Example 10—Discussion

Overall, the characterizations of the LMD microenvironment response to Pembrolizumab demonstrate the complex behavior of cellular ensembles after checkpoint blockade inhibition, and provide a unique window into the dynamic nature of patient response. Applicants find, in particular, that T cells, post-Pembrolizumab across all patients exhibit strong anti-tumor response in the form of interferon-γ signaling. Such behavior is characteristically found in many tumor systems following checkpoint blockade inhibition, and is consistent with the mechanism of drug action. Furthermore, Applicants find that the proportion of proliferating T cells is significantly increased following treatment, suggesting that Pembrolizumab partially recovers this aspect of T cell behavior in LMD. Applicants also find evidence that post-treatment CD8+ T cells are more exhausted than those pre-treatment, but this exhaustion is characteristically heterogeneous, with important clinical implications. In particular, Applicants identify two groups of post-treatment CD8+ T cells: CD8.exh^{hi} and CD8.exh^{lo}, which are consistent with previously described phenotypes. Furthermore, Applicants use the temporally resolved data to demonstrate, in the two patients for which data are available, evolution of the CD8+ T cell microenvironment between these exhausted states, as predicted by other models of CBI-induced T cell reactivation.

Meanwhile, among the innate cells, the response of canonical subpopulations similarly exhibits heterogeneous post-treatment behavior. Applicants find, specifically, that monocyte and macrophage populations appear to support immune-mediated tumor cell killing, through upregulation of antigen processing, as well as expression of specific cytokines and chemokines, and higher levels of M1-like phenotype post-treatment. However, the expression of M2-like phenotype and characteristic genes remain constant. Additionally, Applicants find that classical dendritic cells (cDCs) in particular express immune regulatory behavior post-treatment, including IDO1 which selectively increases in these cells in response to interferon signaling in the microenvironment (REFS). These data indicate that the same intended mechanisms and phenotypes of checkpoint blockade inhibition might only partially recover pro-inflammatory phenotypes, and may have starkly immunosuppressive side effects. Furthermore, this suggests that understanding interactions between rejuvenated T cells and other populations in the immune microenvironment may be critical to fully characterize response to immunotherapy. For example, IDO1 expression in cDCs in particular may require parallel consideration with treatment of Pembrolizumab or other interferon-inducing drugs in LMD or other diseases.

The data set features a heterogeneous cohort with a combination of pre-treatment and post-treatment single-cell samples from different patients. Analysis of these data across all patients maximizes sampling power, detects conserved behavior, and is robust to technical noise, while exploiting the single-cell resolution enabled by scRNA-Seq. These analyses reveal the increased cytotoxic profile of post-treatment T cells, as well as increased interferon response in tumor cells across all patients. The data also includes a smaller, patient-controlled, environment-specific, temporal cohort, which isolates many experimental and patient-extrinsic factors of the larger cohort. These temporal data can help contextualize the dynamics of observations made across the larger cohort, such as the observed change in T cell exhaustion phenotypes over time. Additionally, they can be used to test hypotheses from cross-patient observations, such the non-linear interferon sensitivity in tumor cells of individual patients. Together, these complementary analyses detail a complex, dynamic tumor microenvironment response to Pembrolizumab in LMD, with implications for continuous CBI administration.

Together, these data demonstrate utility in twin-cohort experimental design for this and other studies pairing clinical trials and genomic measurements, where a “patient-maximized” group is supplemented with a smaller but temporally resolved group. Such data offer unique opportunities to achieve different levels of both power and resolution. They enable the discovery of widespread features in response to treatment perturbation, while providing individual case studies to further characterize observations and arrive at refined conclusions. Additionally, Applicants contend that combination of these studies can expand on previously published patient-derived data, including many other clinically founded scRNA-Seq studies.

### T Cell Exhaustion

Taken together, these results suggest that PD1-blockade may—overall—increase the cytotoxic activity and proliferative capacity of CD8+ T cells in the LMD TME. In particular, Applicants identify one subset (CD8.postB) that exhibits high levels of effector function consistent with stem-like, progenitor exhausted state, and another subset (CD8.postA) exhibiting markedly lower levels of effector function, but higher cytotoxicity consistent with terminally exhausted state. Applicants demonstrate that in two patients with LMD for which multiple post-treatment samples are available, terminally exhausted CD8+ T cells predominate later time points, and that the progenitor exhausted phenotype is more closely associated with earlier rather than later treatment.

### Innate

The expression of both pro-inflammatory and anti-inflammatory programs in innate cells as a result of interferon response post-Pembrolizumab treatment suggests that the immune infiltrate in LMD has complex responses to CBI, which depend on microenvironment composition and behavior. In particular, despite pro-inflammatory features in the monocyte/macrophage post-treatment populations, Applicants find that M2-like behavior in these cells may still persist. Additionally, cDCs in this cohort exhibit some behavior that counteracts T cell proliferation. These observations further complicate the interpretation of immune response to immunotherapy and highlight the coordinated interaction between immune populations in clinical setting.

### Tumor Cells Interferon Response

Thus, the data suggest that a single post-treatment measurement may not reliably represent the effect of a drug perturbation in an individual patient. While, robust biological post-perturbation signals can be detected in a cohort with sufficient patient numbers, the characterization of individuals based on their response to drug may be limited with a single post-treatment measurement. Instead, there is substantial and necessary benefit to profiling the post-perturbation condition longitudinally, especially in studies of temporal patient profiles characterized with snapshot measurements such as scRNA-Seq.

### Tumor Subclones

The effect of Pembrolizumab on interferon response in tumor cells is detected here across multiple patients, suggesting that regardless of inter-patient heterogeneity, Pembrolizumab-induced biological signals may be robust enough to characterize. However, profiling the same microenvironment over the course of treatment controls for many external factors, and homogenizes the temporal trajectory to each patient. Analysis of these data demonstrates that, in profiled patients, a single post-treatment time point does not accurately represent the tumor response. Specifically, the data suggest that tumor cell cooperation with enhanced immune activity is transient and that tumor-intrinsic (such as adaptation) or extrinsic mechanisms (IDO1 activity) may be responsible for secondary behavior following initial activity. Furthermore, conclusions from a single post-treatment measurement in certain patients (P029, P043) would result in conclusions that tumor cells do or do not exhibit interferon-γ response, which may not reflect the overall clinical or biological trajectory for that patient.

### Example 11—Tables

TABLE 1
Tumor Samples from Treated vs. Untreated (TvUT) Patients

| CSF.patient.001.TvUT.tumor.bimod.markers.dn. | | | | | | CSF.patient.001.TvUT.tumor.bimod.markers.up.1 | | | | | |
|---|---|---|---|---|---|---|---|---|---|---|---|
| | p_val | avg_logFC | pct.1 | pct.2 | p_val_adj | | p_val | avg_logFC | pct.1 | pct.2 | p_val_adj |
| RNA28S5 | 1.54E−11 | −4.292 | 0.429 | 0.957 | 1.228E−07 | RAB11FIP1 | 1.04E−09 | 1.699 | 0.81 | 0.087 | 8.279E−06 |
| RNA18S5 | 4.36E−09 | −3.980 | 0.429 | 0.957 | 3.485E−05 | BCAP29 | 3.45E−09 | 1.815 | 0.857 | 0 | 2.759E−05 |
| STMN1 | 4.26E−07 | −0.348 | 0.095 | 0.174 | 3.409E−03 | KRT7 | 5.35E−09 | 1.825 | 1 | 0.522 | 4.280E−05 |
| H1FX | 5.91E−07 | −0.346 | 0.095 | 0.174 | 4.729E−03 | ID2 | 1.82E−08 | 2.314 | 0.857 | 0.348 | 1.456E−04 |
| IQGAP2 | 9.13E−07 | −0.420 | 0.238 | 0.174 | 7.304E−03 | ACTG1 | 2.22E−08 | 1.756 | 0.905 | 0.609 | 1.774E−04 |
| RPL13A | 1.53E−06 | −1.559 | 0.619 | 0.913 | 1.223E−02 | NDUFAB1 | 2.28E−08 | 1.546 | 0.905 | 0.043 | 1.820E−04 |
| CD52 | 2.39E−06 | −1.308 | 0.095 | 0.696 | 1.909E−02 | SYAP1 | 2.50E−08 | 1.439 | 0.81 | 0 | 1.998E−04 |
| MTRNR2L5 | 2.49E−06 | −0.776 | 0.095 | 0.739 | 1.988E−02 | ANXA3 | 2.50E−08 | 2.083 | 0.81 | 0 | 1.998E−04 |
| MT-RNR2 | 3.20E−06 | −0.879 | 0.952 | 1 | 2.557E−02 | KIAA1598 | 2.69E−08 | 0.856 | 0.81 | 0.13 | 2.154E−04 |
| RNF13 | 3.30E−06 | −0.371 | 0.333 | 0.087 | 2.639E−02 | CSRP1 | 3.81E−08 | 1.746 | 0.857 | 0.13 | 3.050E−04 |
| MT-RNR1 | 3.79E−06 | −1.641 | 0.714 | 0.913 | 3.028E−02 | TK1 | 1.46E−07 | 1.537 | 0.762 | 0 | 1.167E−03 |
| MTRNR2L13 | 3.91E−06 | −0.287 | 0.095 | 0.652 | 3.124E−02 | HAT1 | 1.46E−07 | 1.528 | 0.762 | 0 | 1.167E−03 |
| MTRNR2L3 | 4.95E−06 | −0.316 | 0.238 | 0.87 | 3.958E−02 | SPTSSB | 1.53E−07 | 1.646 | 0.81 | 0.13 | 1.220E−03 |
| | | | | | | MTRNR2L12 | 1.89E−07 | 0.288 | 0.476 | 0.957 | 1.510E−03 |
| | | | | | | SERF2 | 1.98E−07 | 1.602 | 0.952 | 0.652 | 1.585E−03 |
| | | | | | | GHITM | 2.08E−07 | 0.567 | 0.762 | 0.087 | 1.664E−03 |
| | | | | | | MYL12A | 2.14E−07 | 1.536 | 0.952 | 0.348 | 1.713E−03 |
| | | | | | | PALLD | 2.72E−07 | 1.515 | 0.857 | 0.043 | 2.177E−03 |
| | | | | | | NUDT8 | 2.98E−07 | 1.938 | 0.762 | 0.087 | 2.381E−03 |
| | | | | | | TMEM33 | 3.09E−07 | 0.601 | 0.667 | 0.087 | 2.471E−03 |
| | | | | | | MTRNR2L7 | 5.92E−07 | 0.319 | 0.19 | 0.783 | 4.735E−03 |
| | | | | | | CRNDE | 7.22E−07 | 1.407 | 0.714 | 0 | 5.776E−03 |
| | | | | | | SERINC2 | 7.22E−07 | 0.998 | 0.714 | 0 | 5.776E−03 |
| | | | | | | ACYP2 | 7.22E−07 | 0.880 | 0.714 | 0 | 5.776E−03 |
| | | | | | | MTRNR2L2 | 7.28E−07 | 1.022 | 0.333 | 0.913 | 5.819E−03 |
| | | | | | | LAMTOR5 | 7.80E−07 | 1.305 | 0.857 | 0.087 | 6.241E−03 |
| | | | | | | SPTSSA | 8.70E−07 | 1.076 | 0.905 | 0.13 | 6.961E−03 |
| | | | | | | RNF7 | 9.16E−07 | 1.746 | 0.81 | 0.087 | 7.327E−03 |
| | | | | | | ZNF680 | 9.96E−07 | 0.916 | 0.667 | 0.087 | 7.969E−03 |
| | | | | | | MED11 | 1.14E−06 | 0.513 | 0.571 | 0.087 | 9.092E−03 |
| | | | | | | SPINT2 | 1.39E−06 | 1.648 | 0.857 | 0.522 | 1.108E−02 |
| | | | | | | NT5C3A | 1.50E−06 | 1.676 | 0.81 | 0.043 | 1.202E−02 |
| | | | | | | MGST1 | 1.56E−06 | 1.669 | 0.762 | 0.087 | 1.247E−02 |
| | | | | | | TNFRSF12A | 1.81E−06 | 1.491 | 0.762 | 0.174 | 1.449E−02 |
| | | | | | | MYL6 | 1.96E−06 | 1.613 | 1 | 0.652 | 1.571E−02 |
| | | | | | | CD46 | 2.50E−06 | 1.032 | 0.81 | 0.043 | 1.996E−02 |
| | | | | | | CYR61 | 2.54E−06 | 1.809 | 0.762 | 0.174 | 2.030E−02 |
| | | | | | | TYMP | 2.59E−06 | 1.053 | 0.81 | 0.043 | 2.072E−02 |
| | | | | | | C1orf43 | 2.95E−06 | 0.739 | 0.667 | 0.087 | 2.363E−02 |
| | | | | | | PRDX4 | 2.97E−06 | 1.276 | 0.714 | 0.043 | 2.377E−02 |
| | | | | | | SKIV2L2 | 3.13E−06 | 1.122 | 0.667 | 0 | 2.502E−02 |
| | | | | | | PDCD6 | 3.13E−06 | 1.113 | 0.667 | 0 | 2.502E−02 |
| | | | | | | HAUS2 | 3.13E−06 | 1.008 | 0.667 | 0 | 2.502E−02 |
| | | | | | | COPS3 | 3.13E−06 | 0.863 | 0.667 | 0 | 2.502E−02 |
| | | | | | | ZBTB38 | 3.13E−06 | 0.805 | 0.667 | 0 | 2.502E−02 |
| | | | | | | CCDC59 | 3.13E−06 | 0.855 | 0.667 | 0 | 2.502E−02 |
| | | | | | | MRPL13 | 3.13E−06 | 1.133 | 0.667 | 0 | 2.502E−02 |
| | | | | | | ZNF827 | 3.13E−06 | 0.750 | 0.667 | 0 | 2.502E−02 |
| | | | | | | MSL3 | 3.70E−06 | 0.607 | 0.619 | 0.087 | 2.958E−02 |
| | | | | | | CNIH1 | 4.32E−06 | 1.548 | 0.81 | 0.087 | 3.454E−02 |
| | | | | | | CCNC | 5.12E−06 | 0.627 | 0.762 | 0.13 | 4.092E−02 |

| CSF.patient.014.TvUT.tumor.bimod.markers.dn.1 | | | | | | CSF.patient.014.TvUT.tumor.bimod.markers.up.1 | | | | | |
|---|---|---|---|---|---|---|---|---|---|---|---|
| | p_val | avg_logFC | pct.1 | pct.2 | p_val_adj | | p_val | avg_logFC | pct.1 | pct.2 | p_val_adj |
| MTRNR2L2 | 4.12E−142 | −3.415 | 0.071 | 0.928 | 4.271E−138 | PRKDC | 6.18E−36 | 1.076 | 0.801 | 0.69 | 6.400E−32 |
| CTC- | 1.51E−127 | −2.638 | 0 | 0.809 | 1.562E−123 | H3F3B | 4.34E−32 | 0.836 | 0.848 | 0.716 | 4.495E−28 |
| 338M12.5 | | | | | | | | | | | |
| MTRNR2L12 | 1.29E−104 | −2.473 | 0.062 | 0.812 | 1.339E−100 | CCT6A | 5.26E−31 | 0.963 | 0.72 | 0.536 | 5.445E−27 |
| RNA18S5 | 3.59E−96 | −2.360 | 0.102 | 0.83 | 3.717E−92 | SYNE2 | 6.62E−27 | 0.985 | 0.714 | 0.592 | 6.857E−23 |
| MTRNR2L1 | 1.58E−90 | −2.469 | 0.258 | 0.902 | 1.634E−86 | PRRC2C | 1.46E−25 | 0.789 | 0.724 | 0.645 | 1.508E−21 |
| MT-ND4 | 9.59E−86 | −2.233 | 0.186 | 0.859 | 9.937E−82 | HBB | 3.46E−25 | 1.622 | 0.236 | 0.011 | 3.583E−21 |
| MT-CO3 | 6.51E−82 | −2.142 | 0.186 | 0.836 | 6.746E−78 | MATR3 | 2.42E−23 | 0.794 | 0.652 | 0.485 | 2.505E−19 |
| MTRNR2L3 | 4.18E−74 | −1.902 | 0.081 | 0.729 | 4.334E−70 | RHOA | 2.81E−23 | 0.806 | 0.609 | 0.443 | 2.909E−19 |
| MTRNR2L8 | 8.38E−74 | −2.085 | 0.071 | 0.706 | 8.682E−70 | TPM3 | 5.20E−23 | 0.813 | 0.634 | 0.464 | 5.389E−19 |
| MT-CYB | 2.20E−70 | −1.859 | 0.158 | 0.775 | 2.278E−66 | GDI2 | 5.42E−23 | 0.836 | 0.537 | 0.369 | 5.619E−19 |
| MT-CO2 | 1.12E−64 | −1.813 | 0.143 | 0.737 | 1.164E−60 | ENO1 | 4.91E−22 | 0.722 | 0.677 | 0.584 | 5.082E−18 |
| MT-ATP6 | 2.27E−62 | −1.957 | 0.174 | 0.751 | 2.348E−58 | IPO5 | 8.35E−22 | 0.707 | 0.59 | 0.52 | 8.655E−18 |
| MT-CO1 | 1.93E−61 | −1.839 | 0.252 | 0.806 | 2.003E−57 | HSP90AB1 | 1.59E−21 | 0.703 | 0.795 | 0.671 | 1.649E−17 |
| MT-RNR1 | 6.64E−61 | −1.702 | 0.73 | 0.976 | 6.875E−57 | SPP1 | 1.31E−20 | 0.981 | 0.429 | 0.162 | 1.358E−16 |
| MT-ND2 | 6.66E−61 | −1.820 | 0.264 | 0.809 | 6.899E−57 | CNBP | 1.61E−19 | 0.747 | 0.534 | 0.406 | 1.669E−15 |
| MTRNR2L6 | 1.67E−53 | −1.602 | 0.053 | 0.57 | 1.732E−49 | VCAN | 2.75E−19 | 1.388 | 0.27 | 0.058 | 2.854E−15 |
| RPL35 | 2.97E−52 | −1.214 | 0.724 | 0.928 | 3.076E−48 | TPM4 | 8.47E−19 | 0.791 | 0.736 | 0.597 | 8.778E−15 |
| MT-TS2 | 4.38E−52 | −1.413 | 0.016 | 0.485 | 4.536E−48 | PAPOLA | 2.26E−18 | 0.632 | 0.609 | 0.536 | 2.343E−14 |
| MT-ND1 | 4.93E−52 | −1.466 | 0.087 | 0.618 | 5.105E−48 | SRP72 | 3.54E−18 | 0.710 | 0.547 | 0.432 | 3.670E−14 |
| MTRNR2L11 | 2.47E−50 | −1.188 | 0.059 | 0.568 | 2.561E−46 | CSDE1 | 4.20E−18 | 0.697 | 0.658 | 0.501 | 4.356E−14 |
| MT-RNR2 | 1.14E−46 | −1.565 | 0.972 | 1 | 1.179E−42 | PDIA3 | 4.58E−18 | 0.688 | 0.5 | 0.374 | 4.750E−14 |
| RPL36 | 2.12E−43 | −1.134 | 0.311 | 0.751 | 2.198E−39 | TLN1 | 8.39E−18 | 0.873 | 0.376 | 0.21 | 8.698E−14 |
| RPS18 | 9.97E−43 | −1.206 | 0.72 | 0.883 | 1.033E−38 | SYNCRIP | 9.32E−18 | 0.601 | 0.624 | 0.586 | 9.657E−14 |
| MT-TP | 3.12E−41 | −1.379 | 0.019 | 0.419 | 3.229E−37 | CALM1 | 2.40E−17 | 0.589 | 0.752 | 0.679 | 2.491E−13 |
| RPL28 | 1.05E−37 | −1.052 | 0.463 | 0.788 | 1.090E−33 | BNIP3 | 2.43E−17 | 0.842 | 0.351 | 0.196 | 2.517E−13 |
| RPS27 | 7.25E−36 | −1.111 | 0.186 | 0.599 | 7.510E−32 | SUCO | 5.74E−17 | 0.699 | 0.419 | 0.302 | 5.943E−13 |
| MT-ND5 | 7.48E−36 | −1.258 | 0.127 | 0.565 | 7.747E−32 | HDGF | 6.83E−17 | 0.740 | 0.506 | 0.387 | 7.075E−13 |
| MTRNR2L5 | 6.90E−35 | −0.795 | 0.003 | 0.324 | 7.153E−31 | YWHAZ | 8.18E−17 | 0.555 | 0.714 | 0.66 | 8.470E−13 |
| RPS8 | 9.05E−35 | −0.935 | 0.82 | 0.952 | 9.381E−31 | RAD21 | 1.33E−16 | 0.643 | 0.618 | 0.504 | 1.378E−12 |
| RPS29 | 4.55E−33 | −0.798 | 0.854 | 0.966 | 4.710E−29 | APLP2 | 2.02E−16 | 0.590 | 0.693 | 0.645 | 2.093E−12 |
| RPS19 | 1.03E−32 | −0.952 | 0.811 | 0.926 | 1.071E−28 | HSPD1 | 3.32E−16 | 0.561 | 0.705 | 0.65 | 3.440E−12 |
| RPS9 | 1.74E−32 | −0.967 | 0.376 | 0.729 | 1.798E−28 | EIF5B | 6.44E−16 | 0.690 | 0.624 | 0.488 | 6.675E−12 |
| MT-ND3 | 2.13E−31 | −1.195 | 0.261 | 0.687 | 2.209E−27 | EIF4G1 | 1.02E−15 | 0.669 | 0.497 | 0.395 | 1.059E−11 |
| RPL13 | 2.21E−30 | −0.875 | 0.571 | 0.846 | 2.294E−26 | SMARCC1 | 3.10E−15 | 0.743 | 0.447 | 0.326 | 3.211E−11 |
| MT-ND4L | 3.09E−30 | −1.021 | 0.081 | 0.467 | 3.206E−26 | KPNA4 | 5.29E−15 | 0.694 | 0.413 | 0.241 | 5.482E−11 |
| UBA52 | 3.25E−29 | −0.776 | 0.59 | 0.865 | 3.369E−25 | MRPS35 | 7.42E−15 | 0.650 | 0.615 | 0.512 | 7.691E−11 |
| RPL41 | 5.16E−29 | −0.878 | 0.63 | 0.849 | 5.348E−25 | SLC38A1 | 1.02E−14 | 0.558 | 0.624 | 0.549 | 1.053E−10 |
| RNA28S5 | 6.96E−28 | −0.902 | 0.714 | 0.931 | 7.215E−24 | UAP1 | 2.93E−14 | 0.779 | 0.438 | 0.263 | 3.032E−10 |
| HAP1 | 8.48E−27 | −0.761 | 0.106 | 0.469 | 8.786E−23 | PRPF40A | 4.54E−14 | 0.606 | 0.534 | 0.443 | 4.704E−10 |
| METTL12 | 4.08E−26 | −0.980 | 0.127 | 0.493 | 4.223E−22 | UBR5 | 5.04E−14 | 0.756 | 0.366 | 0.194 | 5.220E−10 |
| MT-ATP8 | 4.05E−24 | −1.103 | 0.112 | 0.456 | 4.191E−20 | RSF1 | 8.98E−14 | 0.615 | 0.391 | 0.34 | 9.303E−10 |
| TCEB3B | 1.05E−23 | −0.485 | 0.019 | 0.273 | 1.093E−19 | CEP350 | 9.00E−14 | 0.686 | 0.41 | 0.337 | 9.326E−10 |
| IFRD2 | 4.24E−23 | −0.764 | 0.037 | 0.324 | 4.391E−19 | SMARCA5 | 9.95E−14 | 0.793 | 0.475 | 0.345 | 1.031E−09 |
| GAS5 | 4.61E−23 | −0.856 | 0.717 | 0.905 | 4.774E−19 | PAK2 | 1.22E−13 | 0.621 | 0.413 | 0.316 | 1.267E−09 |
| RPS21 | 6.14E−23 | −0.695 | 0.817 | 0.952 | 6.366E−19 | CLTC | 1.29E−13 | 0.526 | 0.571 | 0.493 | 1.332E−09 |
| RPL32 | 1.19E−21 | −0.717 | 0.77 | 0.912 | 1.230E−17 | IQGAP1 | 1.74E−13 | 0.622 | 0.478 | 0.371 | 1.803E−09 |
| RPL23A | 1.23E−21 | −0.778 | 0.227 | 0.565 | 1.276E−17 | DDX21 | 1.85E−13 | 0.437 | 0.512 | 0.538 | 1.920E−09 |
| RPL31 | 1.61E−21 | −0.724 | 0.891 | 0.96 | 1.665E−17 | RPL3 | 2.55E−13 | 0.557 | 0.59 | 0.491 | 2.644E−09 |
| DUSP18 | 5.58E−21 | −0.415 | 0.04 | 0.3 | 5.783E−17 | KIF5B | 2.77E−13 | 0.575 | 0.481 | 0.366 | 2.875E−09 |
| EGR1 | 1.86E−20 | −0.738 | 0.121 | 0.446 | 1.924E−16 | SRSF3 | 2.96E−13 | 0.592 | 0.587 | 0.467 | 3.072E−09 |
| MT-TV | 2.61E−20 | −0.469 | 0.009 | 0.228 | 2.708E−16 | SF3B1 | 4.30E−13 | 0.640 | 0.481 | 0.419 | 4.454E−09 |
| RPLP1 | 2.72E−20 | −0.711 | 0.839 | 0.936 | 2.820E−16 | ACSL4 | 9.02E−13 | 0.656 | 0.27 | 0.146 | 9.345E−09 |
| NR4A1 | 3.43E−20 | −0.637 | 0.037 | 0.3 | 3.558E−16 | TAF2 | 1.24E−12 | 0.556 | 0.373 | 0.273 | 1.289E−08 |
| TMSB10 | 8.09E−20 | −0.629 | 0.733 | 0.926 | 8.382E−16 | EMP1 | 1.40E−12 | 0.690 | 0.5 | 0.411 | 1.447E−08 |
| RPS15A | 8.54E−20 | −0.746 | 0.571 | 0.759 | 8.843E−16 | G3BP2 | 1.59E−12 | 0.526 | 0.575 | 0.507 | 1.653E−08 |
| RMRP | 4.55E−19 | −0.793 | 0.078 | 0.358 | 4.713E−15 | EPRS | 1.88E−12 | 0.564 | 0.441 | 0.324 | 1.950E−08 |
| RPL39 | 6.10E−19 | −0.631 | 0.745 | 0.881 | 6.323E−15 | MAN1A2 | 2.33E−12 | 0.772 | 0.429 | 0.241 | 2.415E−08 |
| TMA7 | 1.06E−18 | −0.534 | 0.127 | 0.424 | 1.094E−14 | ABL2 | 2.76E−12 | 0.614 | 0.298 | 0.284 | 2.859E−08 |
| RPS15 | 1.39E−18 | −0.737 | 0.27 | 0.552 | 1.443E−14 | CENPF | 2.98E−12 | 0.786 | 0.463 | 0.443 | 3.088E−08 |
| RPL13A | 2.85E−18 | −0.714 | 0.72 | 0.873 | 2.950E−14 | ARHGEF12 | 3.31E−12 | 0.692 | 0.388 | 0.263 | 3.433E−08 |
| NDUFB7 | 3.86E−18 | −0.629 | 0.189 | 0.485 | 3.997E−14 | ASH1L | 3.40E−12 | 0.602 | 0.425 | 0.284 | 3.525E−08 |
| RPS7 | 8.62E−18 | −0.723 | 0.227 | 0.515 | 8.926E−14 | NUCKS1 | 6.40E−12 | 0.550 | 0.556 | 0.512 | 6.631E−08 |
| FTL | 1.42E−17 | −0.744 | 0.543 | 0.796 | 1.475E−13 | PCM1 | 7.62E−12 | 0.650 | 0.425 | 0.353 | 7.896E−08 |
| FAU | 2.70E−17 | −0.685 | 0.45 | 0.698 | 2.796E−13 | TOMM20 | 8.34E−12 | 0.610 | 0.537 | 0.427 | 8.646E−08 |
| COX6B1 | 7.08E−17 | −0.568 | 0.214 | 0.52 | 7.338E−13 | HNRNPU | 1.32E−11 | 0.419 | 0.556 | 0.578 | 1.363E−07 |
| MZT2B | 1.07E−16 | −0.375 | 0.106 | 0.374 | 1.111E−12 | TSR1 | 2.03E−11 | 0.641 | 0.36 | 0.247 | 2.100E−07 |
| PPDPF | 1.13E−16 | −0.497 | 0.065 | 0.316 | 1.175E−12 | IGFBP3 | 2.65E−11 | 0.792 | 0.478 | 0.371 | 2.742E−07 |
| MT-ND6 | 1.37E−16 | −0.515 | 0.037 | 0.265 | 1.423E−12 | DYNC1H1 | 3.26E−11 | 0.422 | 0.46 | 0.377 | 3.382E−07 |
| PFN1 | 1.58E−16 | −0.562 | 0.401 | 0.687 | 1.642E−12 | MIB1 | 3.85E−11 | 0.552 | 0.342 | 0.26 | 3.994E−07 |
| RPL29 | 2.15E−16 | −0.410 | 0.081 | 0.34 | 2.228E−12 | TROVE2 | 4.08E−11 | 0.562 | 0.382 | 0.31 | 4.231E−07 |
| MTRNR2L7 | 2.18E−16 | −0.457 | 0.006 | 0.183 | 2.260E−12 | EIF4A2 | 5.58E−11 | 0.551 | 0.54 | 0.491 | 5.784E−07 |
| S100A6 | 2.39E−16 | −0.790 | 0.54 | 0.804 | 2.473E−12 | DHRS7 | 6.61E−11 | 0.537 | 0.348 | 0.247 | 6.849E−07 |
| MIF | 3.15E−16 | −0.464 | 0.071 | 0.324 | 3.261E−12 | YWHAE | 6.75E−11 | 0.447 | 0.823 | 0.78 | 6.993E−07 |
| MT-TL1 | 3.28E−16 | −0.381 | 0.025 | 0.223 | 3.400E−12 | B2M | 7.16E−11 | 0.633 | 0.516 | 0.517 | 7.420E−07 |
| HNRNPA1 | 8.49E−16 | −0.657 | 0.292 | 0.592 | 8.799E−12 | CDV3 | 7.27E−11 | 0.594 | 0.379 | 0.271 | 7.537E−07 |
| RPL8 | 1.17E−15 | −0.599 | 0.711 | 0.867 | 1.214E−11 | SSRP1 | 8.59E−11 | 0.519 | 0.379 | 0.318 | 8.901E−07 |
| CFL1 | 1.34E−15 | −0.714 | 0.258 | 0.536 | 1.385E−11 | NBN | 9.39E−11 | 0.604 | 0.388 | 0.337 | 9.726E−07 |
| RPL18 | 3.20E−15 | −0.645 | 0.506 | 0.748 | 3.318E−11 | SENP6 | 9.56E−11 | 0.521 | 0.488 | 0.393 | 9.910E−07 |
| GSTP1 | 3.46E−15 | −0.582 | 0.245 | 0.541 | 3.583E−11 | TLK2 | 1.09E−10 | 0.589 | 0.273 | 0.117 | 1.131E−06 |
| RPL12 | 3.63E−15 | −0.682 | 0.562 | 0.759 | 3.757E−11 | CSNK1A1 | 1.39E−10 | 0.580 | 0.348 | 0.199 | 1.436E−06 |
| RPS3A | 4.38E−15 | −0.622 | 0.196 | 0.467 | 4.539E−11 | RB1CC1 | 1.67E−10 | 0.477 | 0.45 | 0.456 | 1.734E−06 |
| RPS11 | 6.50E−15 | −0.564 | 0.733 | 0.878 | 6.731E−11 | AZINI | 2.02E−10 | 0.327 | 0.5 | 0.541 | 2.094E−06 |
| RPL18A | 1.17E−14 | −0.348 | 0.068 | 0.297 | 1.214E−10 | BAZ1B | 2.03E−10 | 0.589 | 0.398 | 0.337 | 2.101E−06 |
| OST4 | 1.83E−14 | −0.460 | 0.118 | 0.382 | 1.892E−10 | HIST1H1E | 2.30E−10 | 0.503 | 0.227 | 0.194 | 2.381E−06 |
| ANKRD42 | 3.71E−14 | −0.345 | 0.059 | 0.276 | 3.844E−10 | HNRNPA2B1 | 2.69E−10 | 0.480 | 0.839 | 0.782 | 2.787E−06 |
| MYC | 2.01E−13 | −0.589 | 0.252 | 0.541 | 2.080E−09 | ACTG1 | 2.94E−10 | 0.417 | 0.807 | 0.801 | 3.051E−06 |
| POLR2I | 2.24E−13 | −0.355 | 0.078 | 0.302 | 2.320E−09 | STK3 | 3.01E−10 | 0.617 | 0.413 | 0.308 | 3.114E−06 |
| RPL30 | 3.92E−13 | −0.449 | 0.922 | 0.976 | 4.066E−09 | IARS | 3.44E−10 | 0.498 | 0.267 | 0.162 | 3.567E−06 |
| ATP5I | 3.93E−13 | −0.523 | 0.236 | 0.504 | 4.070E−09 | DNAJB1 | 3.90E−10 | 0.406 | 0.239 | 0.387 | 4.036E−06 |
| LHCGR | 3.99E−13 | −0.283 | 0.037 | 0.223 | 4.133E−09 | TAF1D | 4.16E−10 | 0.475 | 0.575 | 0.56 | 4.315E−06 |
| JUNB | 6.63E−13 | −0.533 | 0.115 | 0.358 | 6.873E−09 | PTTG1IP | 4.19E−10 | 0.501 | 0.562 | 0.469 | 4.345E−06 |
| COX7B | 7.65E−13 | −0.623 | 0.261 | 0.515 | 7.927E−09 | YBX1 | 4.83E−10 | 0.520 | 0.571 | 0.491 | 5.007E−06 |
| FXYD5 | 1.58E−12 | −0.362 | 0.053 | 0.255 | 1.639E−08 | TFAM | 5.80E−10 | 0.558 | 0.335 | 0.265 | 6.006E−06 |
| C19orf48 | 1.84E−12 | −0.356 | 0.081 | 0.297 | 1.908E−08 | PTMS | 6.72E−10 | 0.516 | 0.537 | 0.493 | 6.963E−06 |
| ZFP36 | 2.03E−12 | −0.485 | 0.062 | 0.271 | 2.099E−08 | 11-Sep | 7.28E−10 | 0.399 | 0.525 | 0.523 | 7.538E−06 |
| TUBA1A | 2.33E−12 | −0.672 | 0.342 | 0.599 | 2.413E−08 | CCT2 | 8.05E−10 | 0.417 | 0.342 | 0.332 | 8.337E−06 |
| RPS3 | 2.87E−12 | −0.522 | 0.789 | 0.899 | 2.969E−08 | EIF2S1 | 8.73E−10 | 0.489 | 0.354 | 0.294 | 9.041E−06 |
| ZFP36L1 | 3.01E−12 | −0.656 | 0.391 | 0.626 | 3.115E−08 | RAN | 9.94E−10 | 0.377 | 0.543 | 0.546 | 1.030E−05 |
| AP2S1 | 3.49E−12 | −0.262 | 0.099 | 0.308 | 3.619E−08 | TGS1 | 1.14E−09 | 0.608 | 0.37 | 0.255 | 1.186E−05 |
| RPL9 | 4.23E−12 | −0.602 | 0.491 | 0.711 | 4.386E−08 | LCP1 | 1.15E−09 | 0.556 | 0.301 | 0.21 | 1.189E−05 |
| RPL15 | 6.94E−12 | −0.519 | 0.773 | 0.806 | 7.189E−08 | CANX | 1.17E−09 | 0.405 | 0.624 | 0.618 | 1.210E−05 |
| MRPS12 | 8.71E−12 | −0.289 | 0.118 | 0.337 | 9.028E−08 | ETS1 | 1.31E−09 | 0.613 | 0.385 | 0.305 | 1.355E−05 |
| NDUFA11 | 9.59E−12 | −0.420 | 0.193 | 0.43 | 9.940E−08 | SCRN1 | 1.36E−09 | 0.464 | 0.252 | 0.135 | 1.410E−05 |
| RNY1 | 1.91E−11 | −0.276 | 0.025 | 0.186 | 1.982E−07 | DNAJA1 | 1.37E−09 | 0.353 | 0.556 | 0.594 | 1.416E−05 |
| MLF2 | 1.98E−11 | −0.464 | 0.137 | 0.371 | 2.055E−07 | SKP1 | 1.72E−09 | 0.311 | 0.37 | 0.416 | 1.784E−05 |
| ID2 | 2.01E−11 | −0.526 | 0.295 | 0.565 | 2.079E−07 | YWHAB | 1.91E−09 | 0.490 | 0.602 | 0.562 | 1.981E−05 |
| LSM7 | 3.27E−11 | −0.511 | 0.149 | 0.385 | 3.386E−07 | PLIN2 | 2.11E−09 | 0.621 | 0.311 | 0.236 | 2.188E−05 |
| C4orf48 | 3.78E−11 | −0.305 | 0.075 | 0.271 | 3.917E−07 | ATRX | 2.14E−09 | 0.573 | 0.416 | 0.39 | 2.221E−05 |
| MTRNR2L13 | 4.72E−11 | −0.298 | 0.022 | 0.172 | 4.889E−07 | ITGB1 | 2.30E−09 | 0.419 | 0.391 | 0.369 | 2.379E−05 |
| CHCHD2 | 5.43E−11 | −0.495 | 0.255 | 0.512 | 5.629E−07 | MRPL13 | 2.33E−09 | 0.464 | 0.441 | 0.44 | 2.410E−05 |
| NDUFB1 | 5.81E−11 | −0.513 | 0.255 | 0.507 | 6.017E−07 | TTC1 | 2.46E−09 | 0.549 | 0.298 | 0.17 | 2.549E−05 |
| LSM4 | 8.65E−11 | −0.388 | 0.168 | 0.403 | 8.959E−07 | PSAP | 2.48E−09 | 0.462 | 0.376 | 0.332 | 2.566E−05 |
| WDR83OS | 8.93E−11 | −0.351 | 0.13 | 0.353 | 9.251E−07 | PPFIBP1 | 3.01E−09 | 0.601 | 0.357 | 0.22 | 3.116E−05 |
| RPS24 | 9.82E−11 | −0.458 | 0.811 | 0.923 | 1.018E−06 | RARS | 3.09E−09 | 0.537 | 0.286 | 0.191 | 3.204E−05 |
| NDUFA2 | 1.14E−10 | −0.317 | 0.075 | 0.271 | 1.182E−06 | CHD4 | 3.10E−09 | 0.405 | 0.481 | 0.467 | 3.211E−05 |
| PSMB3 | 1.19E−10 | −0.393 | 0.099 | 0.31 | 1.232E−06 | PHF3 | 3.48E−09 | 0.472 | 0.267 | 0.247 | 3.602E−05 |
| S100A11 | 1.39E−10 | −0.463 | 0.332 | 0.586 | 1.445E−06 | MBNL2 | 3.64E−09 | 0.470 | 0.199 | 0.18 | 3.776E−05 |
| ANAPC11 | 1.51E−10 | −0.378 | 0.093 | 0.3 | 1.561E−06 | USO1 | 4.13E−09 | 0.419 | 0.416 | 0.419 | 4.276E−05 |
| RPL26 | 1.92E−10 | −0.502 | 0.63 | 0.806 | 1.986E−06 | ARHGAP5 | 4.14E−09 | 0.544 | 0.314 | 0.194 | 4.284E−05 |
| TTC19 | 1.98E−10 | −0.320 | 0.214 | 0.448 | 2.056E−06 | CAPN2 | 4.18E−09 | 0.542 | 0.335 | 0.233 | 4.332E−05 |
| NDUFB9 | 2.19E−10 | −0.306 | 0.419 | 0.671 | 2.273E−06 | DICER1 | 4.18E−09 | 0.594 | 0.422 | 0.297 | 4.333E−05 |
| LSR | 2.43E−10 | −0.306 | 0.071 | 0.26 | 2.521E−06 | SACS | 4.29E−09 | 0.454 | 0.202 | 0.138 | 4.445E−05 |
| PFDN5 | 2.64E−10 | −0.438 | 0.258 | 0.501 | 2.731E−06 | CHML | 4.32E−09 | 0.589 | 0.472 | 0.382 | 4.477E−05 |
| COX6A1 | 2.71E−10 | −0.454 | 0.267 | 0.504 | 2.810E−06 | ZFR | 4.48E−09 | 0.568 | 0.357 | 0.263 | 4.645E−05 |
| DANCR | 3.02E−10 | −0.312 | 0.224 | 0.456 | 3.128E−06 | JMJD1C | 4.70E−09 | 0.520 | 0.363 | 0.308 | 4.865E−05 |
| CDKN2A | 3.73E−10 | −0.350 | 0.236 | 0.456 | 3.865E−06 | SETX | 4.92E−09 | 0.604 | 0.314 | 0.204 | 5.094E−05 |
| GPX1 | 4.56E−10 | −0.422 | 0.18 | 0.414 | 4.726E−06 | ATP6V1D | 5.33E−09 | 0.446 | 0.239 | 0.125 | 5.520E−05 |
| ATP5J2 | 5.03E−10 | −0.294 | 0.118 | 0.321 | 5.211E−06 | USP14 | 5.49E−09 | 0.524 | 0.348 | 0.241 | 5.686E−05 |
| NDUFB4 | 5.41E−10 | −0.328 | 0.109 | 0.316 | 5.607E−06 | ARPC5 | 5.70E−09 | 0.516 | 0.503 | 0.422 | 5.907E−05 |
| JUND | 5.60E−10 | −0.406 | 0.137 | 0.355 | 5.804E−06 | VCL | 5.77E−09 | 0.523 | 0.311 | 0.212 | 5.983E−05 |
| MRPS26 | 6.01E−10 | −0.285 | 0.068 | 0.249 | 6.230E−06 | CMPK1 | 5.94E−09 | 0.363 | 0.267 | 0.236 | 6.156E−05 |
| ACTN4 | 6.11E−10 | −0.356 | 0.295 | 0.544 | 6.330E−06 | MKI67 | 6.52E−09 | 0.876 | 0.366 | 0.244 | 6.758E−05 |
| NBEAL2 | 9.27E−10 | −0.281 | 0.009 | 0.103 | 9.610E−06 | PDCD10 | 6.63E−09 | 0.502 | 0.475 | 0.432 | 6.868E−05 |
| ARGLU1 | 1.33E−09 | −0.266 | 0.245 | 0.472 | 1.383E−05 | PTPLB | 7.89E−09 | 0.560 | 0.314 | 0.199 | 8.173E−05 |
| CKB | 1.49E−09 | −0.499 | 0.152 | 0.366 | 1.547E−05 | DSTN | 9.64E−09 | 0.381 | 0.537 | 0.533 | 9.984E−05 |
| RAB32 | 1.51E−09 | −0.333 | 0.065 | 0.241 | 1.563E−05 | SPAG9 | 1.03E−08 | 0.634 | 0.354 | 0.225 | 1.064E−04 |
| RPL36AL | 1.80E−09 | −0.422 | 0.168 | 0.387 | 1.867E−05 | TAOK1 | 1.10E−08 | 0.493 | 0.357 | 0.263 | 1.142E−04 |
| CEBPB | 2.09E−09 | −0.371 | 0.059 | 0.225 | 2.167E−05 | CUL5 | 1.48E−08 | 0.548 | 0.388 | 0.345 | 1.532E−04 |
| APH1A | 2.46E−09 | −0.367 | 0.096 | 0.284 | 2.546E−05 | ZNF146 | 1.60E−08 | 0.406 | 0.404 | 0.416 | 1.662E−04 |
| RPS28 | 2.84E−09 | −0.458 | 0.547 | 0.737 | 2.944E−05 | LIMS1 | 1.68E−08 | 0.558 | 0.317 | 0.178 | 1.745E−04 |
| TRIM28 | 4.01E−09 | −0.316 | 0.071 | 0.247 | 4.150E−05 | NOL11 | 1.75E−08 | 0.359 | 0.28 | 0.183 | 1.814E−04 |
| EEF1D | 4.23E−09 | −0.398 | 0.242 | 0.472 | 4.388E−05 | DHX36 | 1.92E−08 | 0.448 | 0.329 | 0.286 | 1.993E−04 |
| SNHG8 | 4.55E−09 | −0.279 | 0.217 | 0.424 | 4.712E−05 | CEBPG | 2.01E−08 | 0.342 | 0.292 | 0.294 | 2.087E−04 |
| TIMM8B | 4.69E−09 | −0.499 | 0.286 | 0.512 | 4.863E−05 | HDAC2 | 2.07E−08 | 0.314 | 0.416 | 0.422 | 2.146E−04 |
| RPL35A | 4.81E−09 | −0.435 | 0.922 | 0.963 | 4.985E−05 | EIF4B | 2.12E−08 | 0.493 | 0.37 | 0.252 | 2.193E−04 |
| ELF3 | 5.06E−09 | −0.261 | 0.102 | 0.284 | 5.238E−05 | BPTF | 2.19E−08 | 0.513 | 0.469 | 0.403 | 2.265E−04 |
| NDUFA13 | 5.43E−09 | −0.488 | 0.398 | 0.61 | 5.622E−05 | TWISTNB | 2.34E−08 | 0.559 | 0.28 | 0.167 | 2.429E−04 |
| TMEM165 | 6.45E−09 | −0.283 | 0.211 | 0.43 | 6.684E−05 | MARK3 | 2.48E−08 | 0.574 | 0.307 | 0.194 | 2.565E−04 |
| FOS | 6.56E−09 | −0.570 | 0.096 | 0.281 | 6.796E−05 | SLC25A5 | 2.85E−08 | 0.437 | 0.643 | 0.525 | 2.956E−04 |
| C14orf2 | 8.32E−09 | −0.436 | 0.307 | 0.536 | 8.621E−05 | GOLGA4 | 2.88E−08 | 0.419 | 0.413 | 0.459 | 2.985E−04 |
| PHB2 | 8.86E−09 | −0.392 | 0.18 | 0.39 | 9.178E−05 | CERS6 | 4.01E−08 | 0.476 | 0.239 | 0.18 | 4.155E−04 |
| ABHD11 | 9.13E−09 | −0.276 | 0.022 | 0.151 | 9.458E−05 | GLTSCR2 | 4.10E−08 | 0.456 | 0.432 | 0.369 | 4.249E−04 |
| CLPP | 1.01E−08 | −0.285 | 0.071 | 0.239 | 1.045E−04 | ADAR | 4.40E−08 | 0.511 | 0.332 | 0.257 | 4.560E−04 |
| CSNK2B | 1.16E−08 | −0.286 | 0.065 | 0.231 | 1.198E−04 | SPEN | 4.41E−08 | 0.453 | 0.264 | 0.175 | 4.568E−04 |
| S100A13 | 1.26E−08 | −0.501 | 0.242 | 0.459 | 1.303E−04 | DDX42 | 4.58E−08 | 0.514 | 0.292 | 0.21 | 4.746E−04 |
| PCBP2 | 1.53E−08 | −0.418 | 0.186 | 0.401 | 1.590E−04 | KIDINS220 | 5.02E−08 | 0.535 | 0.267 | 0.18 | 5.206E−04 |
| SSR4 | 1.59E−08 | −0.354 | 0.152 | 0.355 | 1.646E−04 | H2AFY | 5.64E−08 | 0.409 | 0.357 | 0.318 | 5.843E−04 |
| LAMP1 | 1.66E−08 | −0.397 | 0.143 | 0.345 | 1.718E−04 | EIF4G2 | 6.07E−08 | 0.293 | 0.447 | 0.488 | 6.292E−04 |
| RPL10 | 1.67E−08 | −0.380 | 0.199 | 0.416 | 1.734E−04 | HNRNPM | 6.53E−08 | 0.341 | 0.469 | 0.485 | 6.769E−04 |
| ANKRD10 | 1.69E−08 | −0.258 | 0.14 | 0.332 | 1.755E−04 | BROX | 6.91E−08 | 0.353 | 0.205 | 0.164 | 7.162E−04 |
| UQCRQ | 1.76E−08 | −0.476 | 0.295 | 0.517 | 1.821E−04 | EIF3D | 7.80E−08 | 0.458 | 0.329 | 0.279 | 8.078E−04 |
| RPN2 | 1.87E−08 | −0.326 | 0.205 | 0.419 | 1.933E−04 | PICALM | 7.94E−08 | 0.478 | 0.32 | 0.212 | 8.225E−04 |
| TUBA1B | 1.94E−08 | −0.444 | 0.326 | 0.552 | 2.012E−04 | USP1 | 7.96E−08 | 0.446 | 0.425 | 0.39 | 8.252E−04 |
| IER2 | 2.89E−08 | −0.372 | 0.18 | 0.39 | 2.991E−04 | CHEK1 | 8.36E−08 | 0.461 | 0.264 | 0.188 | 8.661E−04 |
| OAZ1 | 6.86E−08 | −0.381 | 0.261 | 0.48 | 7.107E−04 | CWC15 | 9.14E−08 | 0.400 | 0.432 | 0.401 | 9.474E−04 |
| NBPF16 | 7.05E−08 | −0.272 | 0.028 | 0.133 | 7.305E−04 | MOB1B | 9.34E−08 | 0.463 | 0.332 | 0.241 | 9.682E−04 |
| RPL27 | 9.43E−08 | −0.448 | 0.929 | 0.936 | 9.766E−04 | NOP56 | 9.62E−08 | 0.392 | 0.503 | 0.504 | 9.966E−04 |
| ATP5E | 1.04E−07 | −0.291 | 0.432 | 0.631 | 1.075E−03 | TPX2 | 9.73E−08 | 0.539 | 0.342 | 0.284 | 1.008E−03 |
| POLR2J | 1.14E−07 | −0.259 | 0.053 | 0.199 | 1.179E−03 | STAG2 | 9.78E−08 | 0.263 | 0.261 | 0.321 | 1.013E−03 |
| COPRS | 1.21E−07 | −0.348 | 0.037 | 0.164 | 1.254E−03 | TAX1BP1 | 9.80E−08 | 0.384 | 0.363 | 0.353 | 1.015E−03 |
| RBX1 | 1.26E−07 | −0.327 | 0.099 | 0.271 | 1.304E−03 | CHMP1B | 1.22E−07 | 0.268 | 0.189 | 0.204 | 1.259E−03 |
| GNG5 | 1.37E−07 | −0.266 | 0.112 | 0.284 | 1.415E−03 | CAPRIN1 | 1.23E−07 | 0.432 | 0.329 | 0.257 | 1.272E−03 |
| JUP | 1.50E−07 | −0.267 | 0.087 | 0.252 | 1.558E−03 | DHX40 | 1.24E−07 | 0.495 | 0.314 | 0.244 | 1.282E−03 |
| PCBP1 | 1.78E−07 | −0.277 | 0.102 | 0.271 | 1.847E−03 | MIER1 | 1.31E−07 | 0.452 | 0.186 | 0.138 | 1.357E−03 |
| ELP5 | 2.25E−07 | −0.292 | 0.09 | 0.252 | 2.327E−03 | SRGN | 1.31E−07 | 0.691 | 0.127 | 0.042 | 1.359E−03 |
| UBL5 | 2.35E−07 | −0.441 | 0.624 | 0.735 | 2.436E−03 | SBNO1 | 1.33E−07 | 0.534 | 0.298 | 0.172 | 1.377E−03 |
| RHOC | 2.38E−07 | −0.281 | 0.071 | 0.225 | 2.463E−03 | CYCS | 1.34E−07 | 0.319 | 0.512 | 0.538 | 1.387E−03 |
| MRPL12 | 2.60E−07 | −0.279 | 0.099 | 0.265 | 2.693E−03 | 7-Mar | 1.38E−07 | 0.497 | 0.314 | 0.239 | 1.431E−03 |
| RCN2 | 2.85E−07 | −0.294 | 0.168 | 0.358 | 2.948E−03 | FAM111B | 1.46E−07 | 0.584 | 0.298 | 0.244 | 1.517E−03 |
| MRPL4 | 4.41E−07 | −0.295 | 0.081 | 0.233 | 4.571E−03 | GMPS | 1.70E−07 | 0.486 | 0.242 | 0.149 | 1.756E−03 |
| C12orf57 | 5.18E−07 | −0.392 | 0.401 | 0.602 | 5.371E−03 | SRRM2 | 1.92E−07 | 0.466 | 0.335 | 0.202 | 1.989E−03 |
| EEF1B2 | 6.50E−07 | −0.394 | 0.394 | 0.573 | 6.730E−03 | UBAP2 | 2.05E−07 | 0.503 | 0.326 | 0.276 | 2.121E−03 |
| RPL37A | 6.79E−07 | −0.384 | 0.804 | 0.881 | 7.035E−03 | LRPPRC | 2.06E−07 | 0.398 | 0.295 | 0.271 | 2.133E−03 |
| HES1 | 7.54E−07 | −0.450 | 0.205 | 0.39 | 7.809E−03 | HSPH1 | 2.39E−07 | 0.297 | 0.196 | 0.271 | 2.477E−03 |
| RPS27A | 7.90E−07 | −0.389 | 0.512 | 0.7 | 8.182E−03 | STRBP | 2.41E−07 | 0.440 | 0.23 | 0.146 | 2.500E−03 |
| ATP5L | 7.96E−07 | −0.383 | 0.416 | 0.615 | 8.248E−03 | RIF1 | 2.64E−07 | 0.469 | 0.466 | 0.43 | 2.734E−03 |
| NDUFB2 | 8.20E−07 | −0.291 | 0.273 | 0.472 | 8.494E−03 | BZW1 | 2.66E−07 | 0.413 | 0.311 | 0.273 | 2.761E−03 |
| SNHG6 | 8.33E−07 | −0.415 | 0.602 | 0.74 | 8.632E−03 | CBX5 | 3.39E−07 | 0.374 | 0.301 | 0.257 | 3.514E−03 |
| PDCD5 | 8.91E−07 | −0.278 | 0.292 | 0.491 | 9.229E−03 | ANKRD17 | 4.02E−07 | 0.412 | 0.354 | 0.332 | 4.170E−03 |
| TAGLN2 | 9.30E−07 | −0.390 | 0.289 | 0.491 | 9.631E−03 | MORF4L2 | 4.27E−07 | 0.353 | 0.453 | 0.395 | 4.420E−03 |
| HNRNPA0 | 9.42E−07 | −0.255 | 0.152 | 0.329 | 9.759E−03 | PHF14 | 4.33E−07 | 0.379 | 0.28 | 0.289 | 4.482E−03 |
| H2AFX | 9.95E−07 | −0.278 | 0.068 | 0.204 | 1.031E−02 | BUB1 | 4.73E−07 | 0.515 | 0.227 | 0.119 | 4.897E−03 |
| COX7A2 | 1.03E−06 | −0.413 | 0.435 | 0.626 | 1.069E−02 | VRK1 | 4.91E−07 | 0.465 | 0.255 | 0.199 | 5.089E−03 |
| CHCHD3 | 1.03E−06 | −0.253 | 0.14 | 0.31 | 1.070E−02 | PSME4 | 4.92E−07 | 0.532 | 0.22 | 0.117 | 5.096E−03 |
| C17orf76- | 1.09E−06 | −0.376 | 0.494 | 0.679 | 1.127E−02 | MLTK | 4.99E−07 | 0.370 | 0.245 | 0.233 | 5.168E−03 |
| AS1 | | | | | | | | | | | |
| COX5B | 1.30E−06 | −0.257 | 0.23 | 0.419 | 1.345E−02 | ABI2 | 4.99E−07 | 0.427 | 0.307 | 0.231 | 5.174E−03 |
| RPL34 | 1.36E−06 | −0.426 | 0.537 | 0.679 | 1.404E−02 | TKT | 5.11E−07 | 0.320 | 0.255 | 0.268 | 5.298E−03 |
| CYP1B1 | 1.89E−06 | −0.435 | 0.059 | 0.191 | 1.955E−02 | BIRC2 | 5.20E−07 | 0.476 | 0.398 | 0.345 | 5.386E−03 |
| LAPTM4A | 2.10E−06 | −0.323 | 0.174 | 0.353 | 2.173E−02 | SNX6 | 5.72E−07 | 0.475 | 0.276 | 0.17 | 5.923E−03 |
| SNHG5 | 2.31E−06 | −0.250 | 0.789 | 0.92 | 2.393E−02 | RSL1D1 | 5.81E−07 | 0.451 | 0.425 | 0.398 | 6.016E−03 |
| RPL27A | 2.78E−06 | −0.373 | 0.714 | 0.82 | 2.881E−02 | USP13 | 5.97E−07 | 0.335 | 0.143 | 0.058 | 6.187E−03 |
| TSC22D1 | 2.85E−06 | −0.389 | 0.233 | 0.408 | 2.953E−02 | HLA-E | 6.07E−07 | 0.495 | 0.323 | 0.215 | 6.290E−03 |
| NR4A2 | 3.07E−06 | −0.421 | 0.068 | 0.196 | 3.180E−02 | FAM208B | 6.19E−07 | 0.524 | 0.311 | 0.231 | 6.415E−03 |
| NDUFS5 | 3.19E−06 | −0.298 | 0.329 | 0.528 | 3.300E−02 | VEZT | 6.44E−07 | 0.254 | 0.149 | 0.18 | 6.674E−03 |
| VIMP | 3.93E−06 | −0.309 | 0.109 | 0.263 | 4.067E−02 | PTPN11 | 6.68E−07 | 0.375 | 0.311 | 0.255 | 6.922E−03 |
| CTNNB1 | 3.94E−06 | −0.360 | 0.404 | 0.576 | 4.083E−02 | RAB13 | 6.82E−07 | 0.378 | 0.255 | 0.204 | 7.069E−03 |
| ACTB | 4.01E−06 | −0.323 | 0.457 | 0.653 | 4.156E−02 | PPP2R2A | 6.87E−07 | 0.447 | 0.311 | 0.265 | 7.113E−03 |
| | | | | | | SCARB2 | 7.19E−07 | 0.309 | 0.317 | 0.329 | 7.454E−03 |
| | | | | | | KMT2A | 9.00E−07 | 0.442 | 0.366 | 0.297 | 9.320E−03 |
| | | | | | | LMO7 | 9.55E−07 | 0.475 | 0.224 | 0.135 | 9.894E−03 |
| | | | | | | MACF1 | 9.84E−07 | 0.514 | 0.388 | 0.286 | 1.020E−02 |
| | | | | | | NUDCD1 | 1.02E−06 | 0.383 | 0.373 | 0.353 | 1.052E−02 |
| | | | | | | SON | 1.05E−06 | 0.338 | 0.5 | 0.499 | 1.083E−02 |
| | | | | | | SFXN1 | 1.06E−06 | 0.331 | 0.165 | 0.125 | 1.094E−02 |
| | | | | | | DNAJC3 | 1.10E−06 | 0.447 | 0.273 | 0.244 | 1.142E−02 |
| | | | | | | HIST1H1C | 1.11E−06 | 0.467 | 0.252 | 0.231 | 1.152E−02 |
| | | | | | | HSPA5 | 1.12E−06 | 0.509 | 0.565 | 0.538 | 1.157E−02 |
| | | | | | | SMC4 | 1.13E−06 | 0.455 | 0.488 | 0.44 | 1.174E−02 |
| | | | | | | EIF3A | 1.17E−06 | 0.420 | 0.472 | 0.416 | 1.216E−02 |
| | | | | | | ZCCHC11 | 1.19E−06 | 0.507 | 0.326 | 0.236 | 1.228E−02 |
| | | | | | | LARS | 1.21E−06 | 0.480 | 0.323 | 0.289 | 1.256E−02 |
| | | | | | | UTP23 | 1.22E−06 | 0.452 | 0.401 | 0.366 | 1.268E−02 |
| | | | | | | ILF3 | 1.43E−06 | 0.368 | 0.568 | 0.525 | 1.486E−02 |
| | | | | | | SMEK1 | 1.51E−06 | 0.323 | 0.363 | 0.353 | 1.565E−02 |
| | | | | | | SSR3 | 1.57E−06 | 0.293 | 0.32 | 0.345 | 1.628E−02 |
| | | | | | | UBXN4 | 1.58E−06 | 0.406 | 0.416 | 0.332 | 1.636E−02 |
| | | | | | | TM9SF3 | 1.59E−06 | 0.360 | 0.444 | 0.43 | 1.646E−02 |
| | | | | | | CD109 | 1.68E−06 | 0.397 | 0.323 | 0.225 | 1.737E−02 |
| | | | | | | NSMCE2 | 1.70E−06 | 0.363 | 0.214 | 0.167 | 1.758E−02 |
| | | | | | | PPM1G | 1.78E−06 | 0.264 | 0.258 | 0.281 | 1.844E−02 |
| | | | | | | SETD2 | 1.97E−06 | 0.477 | 0.267 | 0.186 | 2.041E−02 |
| | | | | | | YES1 | 1.98E−06 | 0.395 | 0.348 | 0.297 | 2.052E−02 |
| | | | | | | ROCK1 | 2.05E−06 | 0.389 | 0.398 | 0.363 | 2.126E−02 |
| | | | | | | RDX | 2.08E−06 | 0.307 | 0.339 | 0.374 | 2.156E−02 |
| | | | | | | PPP2R2D | 2.09E−06 | 0.320 | 0.127 | 0.08 | 2.168E−02 |
| | | | | | | CPSF2 | 2.22E−06 | 0.420 | 0.339 | 0.263 | 2.303E−02 |
| | | | | | | RBM25 | 2.24E−06 | 0.432 | 0.466 | 0.435 | 2.322E−02 |
| | | | | | | LYZ | 2.38E−06 | 0.635 | 0.18 | 0.09 | 2.463E−02 |
| | | | | | | PRRC2B | 2.40E−06 | 0.374 | 0.236 | 0.215 | 2.484E−02 |
| | | | | | | ZFYVE16 | 2.40E−06 | 0.267 | 0.118 | 0.135 | 2.490E−02 |
| | | | | | | HADHA | 2.44E−06 | 0.488 | 0.32 | 0.188 | 2.530E−02 |
| | | | | | | MX2 | 2.56E−06 | 0.350 | 0.112 | 0.056 | 2.653E−02 |
| | | | | | | LDHB | 2.59E−06 | 0.370 | 0.643 | 0.607 | 2.687E−02 |
| | | | | | | CKAP5 | 2.62E−06 | 0.478 | 0.286 | 0.186 | 2.712E−02 |
| | | | | | | KEAP1 | 2.69E−06 | 0.267 | 0.23 | 0.244 | 2.784E−02 |
| | | | | | | ATP1A1 | 2.69E−06 | 0.431 | 0.457 | 0.411 | 2.785E−02 |
| | | | | | | NOL7 | 2.76E−06 | 0.275 | 0.366 | 0.393 | 2.858E−02 |
| | | | | | | IPO7 | 2.76E−06 | 0.356 | 0.382 | 0.358 | 2.862E−02 |
| | | | | | | CTC-425F1.4 | 2.79E−06 | 0.434 | 0.252 | 0.164 | 2.889E−02 |
| | | | | | | RNF115 | 2.82E−06 | 0.359 | 0.189 | 0.164 | 2.918E−02 |
| | | | | | | ARL6IP1 | 2.83E−06 | 0.495 | 0.317 | 0.255 | 2.932E−02 |
| | | | | | | HNRNPK | 2.87E−06 | 0.320 | 0.453 | 0.451 | 2.977E−02 |
| | | | | | | TRAM1 | 2.91E−06 | 0.285 | 0.543 | 0.544 | 3.020E−02 |
| | | | | | | CCT4 | 3.00E−06 | 0.417 | 0.304 | 0.265 | 3.108E−02 |
| | | | | | | CAP1 | 3.02E−06 | 0.461 | 0.391 | 0.321 | 3.125E−02 |
| | | | | | | ARF1 | 3.03E−06 | 0.301 | 0.5 | 0.499 | 3.136E−02 |
| | | | | | | SH3GLB1 | 3.12E−06 | 0.362 | 0.311 | 0.297 | 3.229E−02 |
| | | | | | | HUWE1 | 3.14E−06 | 0.387 | 0.363 | 0.302 | 3.249E−02 |
| | | | | | | MCMBP | 3.29E−06 | 0.432 | 0.301 | 0.233 | 3.413E−02 |
| | | | | | | C6orf48 | 3.65E−06 | 0.445 | 0.571 | 0.517 | 3.784E−02 |
| | | | | | | RAB10 | 3.68E−06 | 0.336 | 0.267 | 0.265 | 3.817E−02 |
| | | | | | | FTH1 | 3.83E−06 | 0.314 | 0.59 | 0.597 | 3.971E−02 |
| | | | | | | LBR | 3.85E−06 | 0.488 | 0.317 | 0.236 | 3.984E−02 |
| | | | | | | DNAJC10 | 3.93E−06 | 0.447 | 0.354 | 0.289 | 4.076E−02 |
| | | | | | | ACIN1 | 3.94E−06 | 0.426 | 0.239 | 0.135 | 4.080E−02 |
| | | | | | | HSPA1A | 3.94E−06 | 0.259 | 0.093 | 0.178 | 4.085E−02 |
| | | | | | | WHSC1 | 4.03E−06 | 0.409 | 0.326 | 0.252 | 4.180E−02 |
| | | | | | | ABCF1 | 4.25E−06 | 0.416 | 0.398 | 0.353 | 4.400E−02 |
| | | | | | | MOXD1 | 4.38E−06 | 0.261 | 0.177 | 0.194 | 4.540E−02 |
| | | | | | | DDX46 | 4.50E−06 | 0.338 | 0.301 | 0.316 | 4.664E−02 |
| | | | | | | CLIC4 | 4.70E−06 | 0.355 | 0.252 | 0.239 | 4.875E−02 |
| | | | | | | PIK3R1 | 4.72E−06 | 0.569 | 0.127 | 0.074 | 4.887E−02 |
| | | | | | | ATL3 | 4.76E−06 | 0.388 | 0.217 | 0.175 | 4.933E−02 |
| | | | | | | PMAIP1 | 4.78E−06 | 0.280 | 0.112 | 0.117 | 4.957E−02 |

| CSF.patient.029.TvUT.tumor.bimod.markers.dn.1 | | | | | | CSF.patient.029.TvUT.tumor.bimod.markers.up.1 | | | | | |
|---|---|---|---|---|---|---|---|---|---|---|---|
| | p_val | avg_logFC | pct.1 | pct.2 | p_val_adj | | p_val | avg_logFC | pct.1 | pct.2 | p_val_adj |
| MTRNR2L8 | 3.20534969052426e−318 | −5.008 | 0.803 | 0.947 | 3.12361327341589e−314 | TPT1 | 1.06E−64 | 1.462 | 0.988 | 0.697 | 1.034E−60 |
| MTRNR2L2 | 3.09E−267 | −3.834 | 0.902 | 0.912 | 3.015E−263 | ATP5H | 1.01E−57 | 0.588 | 0.855 | 0.32 | 9.882E−54 |
| MTRNR2L1 | 7.31E−257 | −3.790 | 0.933 | 0.965 | 7.123E−253 | RBM3 | 2.40E−51 | 0.813 | 0.787 | 0.276 | 2.339E−47 |
| MTRNR2L12 | 1.20E−252 | −3.851 | 0.902 | 0.934 | 1.165E−248 | EEF2 | 4.70E−51 | 0.398 | 0.873 | 0.399 | 4.581E−47 |
| MTRNR2L3 | 1.17E−224 | −3.824 | 0.703 | 0.737 | 1.143E−220 | AMD1 | 4.21E−49 | 0.292 | 0.871 | 0.434 | 4.102E−45 |
| RPS27 | 1.10E−190 | −3.033 | 0.702 | 0.654 | 1.075E−186 | TOMM20 | 6.69E−48 | 0.541 | 0.912 | 0.491 | 6.521E−44 |
| MIF | 2.52E−177 | −2.852 | 0.726 | 0.697 | 2.455E−173 | RHOA | 9.11E−46 | 0.840 | 0.868 | 0.386 | 8.876E−42 |
| HAP1 | 3.00E−177 | −2.081 | 0.852 | 0.561 | 2.919E−173 | EIF3H | 1.77E−45 | 0.469 | 0.85 | 0.434 | 1.721E−41 |
| MTRNR2L6 | 1.25E−175 | −2.852 | 0.643 | 0.539 | 1.215E−171 | RBP7 | 2.46E−45 | 0.829 | 0.735 | 0.25 | 2.401E−41 |
| RPL10 | 3.24E−162 | −2.681 | 0.67 | 0.57 | 3.154E−158 | SUCLG1 | 4.90E−45 | 0.734 | 0.805 | 0.329 | 4.773E−41 |
| COX6A1P2 | 8.04E−150 | −2.406 | 0.625 | 0.425 | 7.836E−146 | SOD1 | 1.84E−44 | 0.488 | 0.794 | 0.307 | 1.796E−40 |
| MTRNR2L7 | 8.50E−147 | −3.496 | 0.345 | 0.504 | 8.284E−143 | SSU72 | 6.94E−44 | 0.422 | 0.681 | 0.219 | 6.768E−40 |
| EDARADD | 1.03E−144 | −2.402 | 0.469 | 0.303 | 9.990E−141 | COA6 | 1.64E−43 | 0.733 | 0.664 | 0.171 | 1.598E−39 |
| MTRNR2L13 | 2.50E−142 | −3.478 | 0.284 | 0.408 | 2.438E−138 | TMEM165 | 2.66E−43 | 1.469 | 0.784 | 0.289 | 2.590E−39 |
| PABPC3 | 2.77E−137 | −2.634 | 0.387 | 0.333 | 2.701E−133 | UGP2 | 3.09E−43 | 0.514 | 0.868 | 0.452 | 3.008E−39 |
| TMSB4X | 4.67E−133 | −2.074 | 0.967 | 0.934 | 4.548E−129 | CLTA | 3.55E−43 | 0.633 | 0.672 | 0.202 | 3.458E−39 |
| MTRNR2L11 | 4.67E−128 | −2.854 | 0.369 | 0.303 | 4.549E−124 | KRT7 | 5.35E−43 | 0.809 | 0.936 | 0.601 | 5.216E−39 |
| ATP5EP2 | 6.38E−123 | −2.158 | 0.511 | 0.259 | 6.213E−119 | COPB2 | 6.16E−43 | 0.419 | 0.722 | 0.263 | 6.006E−39 |
| TMA7 | 1.55E−122 | −2.049 | 0.616 | 0.386 | 1.514E−118 | ZFAS1 | 1.44E−42 | 0.412 | 0.654 | 0.193 | 1.406E−38 |
| RPL23A | 2.83E−119 | −1.985 | 0.682 | 0.425 | 2.754E−115 | HDGF | 2.70E−42 | 0.499 | 0.782 | 0.333 | 2.627E−38 |
| HMGB1 | 4.54E−119 | −1.962 | 0.874 | 0.715 | 4.428E−115 | VBP1 | 3.17E−42 | 0.661 | 0.764 | 0.298 | 3.088E−38 |
| MT-CO2 | 1.62E−118 | −2.328 | 0.958 | 0.908 | 1.581E−114 | APEX1 | 1.20E−41 | 0.670 | 0.684 | 0.232 | 1.171E−37 |
| RPS7 | 1.84E−115 | −1.947 | 0.569 | 0.303 | 1.794E−111 | SSR3 | 1.36E−41 | 0.337 | 0.811 | 0.368 | 1.323E−37 |
| RPSA | 8.58E−115 | −1.695 | 0.716 | 0.39 | 8.365E−111 | NGFRAP1 | 1.42E−41 | 0.258 | 0.655 | 0.232 | 1.379E−37 |
| MTRNR2L5 | 3.20E−111 | −2.220 | 0.483 | 0.281 | 3.114E−107 | EID1 | 1.43E−41 | 0.461 | 0.604 | 0.167 | 1.389E−37 |
| RPS28 | 3.70E−111 | −1.509 | 0.722 | 0.355 | 3.609E−107 | POLR1D | 2.48E−41 | 0.443 | 0.669 | 0.232 | 2.418E−37 |
| RPL41 | 7.22E−106 | −1.612 | 0.864 | 0.614 | 7.040E−102 | ATP5G3 | 7.65E−41 | 0.413 | 0.761 | 0.311 | 7.452E−37 |
| RPL12 | 1.36E−104 | −2.002 | 0.867 | 0.838 | 1.327E−100 | TCP1 | 7.88E−41 | 0.714 | 0.687 | 0.232 | 7.683E−37 |
| RPL6 | 6.18E−103 | −1.581 | 0.759 | 0.39 | 6.025E−99 | GADD45GIP1 | 1.89E−40 | 0.675 | 0.682 | 0.206 | 1.840E−36 |
| RPS10 | 1.32E−99 | −1.021 | 0.755 | 0.259 | 1.290E−95 | CD55 | 3.37E−40 | 1.045 | 0.703 | 0.232 | 3.288E−36 |
| SNRPG | 1.49E−97 | −1.674 | 0.637 | 0.404 | 1.455E−93 | CMPK1 | 4.09E−40 | 0.683 | 0.708 | 0.254 | 3.988E−36 |
| RPS25 | 4.50E−97 | −1.443 | 0.678 | 0.32 | 4.388E−93 | ILF2 | 4.17E−40 | 0.396 | 0.821 | 0.434 | 4.062E−36 |
| FTH1 | 1.02E−96 | −1.620 | 0.933 | 0.868 | 9.984E−93 | OPTN | 1.03E−39 | 0.482 | 0.59 | 0.175 | 1.002E−35 |
| RPS18 | 1.53E−95 | −1.682 | 0.98 | 0.952 | 1.491E−91 | CTNNA1 | 1.10E−39 | 0.484 | 0.7 | 0.259 | 1.071E−35 |
| RPL13A | 8.94E−95 | −1.607 | 0.915 | 0.737 | 8.716E−91 | SFRP1 | 1.39E−39 | 0.287 | 0.693 | 0.285 | 1.355E−35 |
| RPL21 | 1.16E−94 | −2.507 | 0.362 | 0.364 | 1.130E−90 | DAP | 1.89E−39 | 0.483 | 0.694 | 0.259 | 1.840E−35 |
| RPS3A | 3.40E−94 | −1.825 | 0.501 | 0.232 | 3.314E−90 | C1orf21 | 3.04E−39 | 0.590 | 0.772 | 0.351 | 2.965E−35 |
| RPL9 | 1.42E−93 | −1.339 | 0.759 | 0.425 | 1.385E−89 | CAPN2 | 5.47E−39 | 0.645 | 0.896 | 0.518 | 5.335E−35 |
| RPL28 | 7.55E−90 | −1.010 | 0.864 | 0.491 | 7.354E−86 | TCF25 | 8.46E−39 | 0.421 | 0.622 | 0.184 | 8.245E−35 |
| H3F3A | 1.05E−89 | −2.171 | 0.307 | 0.211 | 1.027E−85 | NCL | 9.08E−39 | 0.259 | 0.914 | 0.575 | 8.852E−35 |
| RPL22 | 1.73E−88 | −1.193 | 0.702 | 0.32 | 1.690E−84 | EIF2AK1 | 1.32E−38 | 0.544 | 0.629 | 0.193 | 1.282E−34 |
| C11orf31 | 1.73E−85 | −1.368 | 0.632 | 0.285 | 1.689E−81 | LAMP2 | 2.62E−38 | 0.684 | 0.829 | 0.395 | 2.554E−34 |
| RPL18A | 1.39E−82 | −1.805 | 0.337 | 0.171 | 1.356E−78 | LMAN1 | 2.64E−38 | 0.598 | 0.703 | 0.281 | 2.568E−34 |
| LY6E | 7.76E−82 | −1.540 | 0.776 | 0.553 | 7.564E−78 | S100A10 | 2.72E−38 | 0.376 | 0.868 | 0.522 | 2.650E−34 |
| PPIA | 8.18E−82 | −1.016 | 0.817 | 0.399 | 7.969E−78 | MT-ND3 | 3.51E−38 | 0.746 | 0.982 | 0.675 | 3.422E−34 |
| RPS15 | 1.62E−81 | −1.263 | 0.803 | 0.518 | 1.576E−77 | H2AFY | 6.16E−38 | 0.533 | 0.646 | 0.215 | 6.000E−34 |
| EIF1AX | 1.59E−80 | −1.385 | 0.613 | 0.285 | 1.550E−76 | PTBP3 | 7.20E−38 | 0.438 | 0.741 | 0.333 | 7.020E−34 |
| MT-ATP8 | 4.00E−80 | −1.487 | 0.805 | 0.583 | 3.899E−76 | CHMP2B | 9.73E−38 | 0.733 | 0.673 | 0.232 | 9.486E−34 |
| PFN1 | 6.37E−80 | −1.431 | 0.746 | 0.539 | 6.204E−76 | HNRNPDL | 1.15E−37 | 0.311 | 0.614 | 0.254 | 1.123E−33 |
| H3F3C | 2.05E−79 | −2.051 | 0.2 | 0.154 | 1.994E−75 | SRRM2 | 1.47E−37 | 0.255 | 0.533 | 0.132 | 1.435E−33 |
| PPDPF | 3.62E−79 | −1.386 | 0.752 | 0.535 | 3.530E−75 | CCT4 | 1.93E−37 | 0.325 | 0.663 | 0.246 | 1.883E−33 |
| RPL29 | 9.28E−79 | −1.389 | 0.605 | 0.298 | 9.048E−75 | SAT1 | 2.58E−37 | 0.640 | 0.787 | 0.404 | 2.514E−33 |
| NPM1 | 1.44E−78 | −1.093 | 0.803 | 0.461 | 1.403E−74 | SUGT1 | 2.99E−37 | 0.884 | 0.592 | 0.149 | 2.909E−33 |
| UBBP4 | 5.08E−78 | −2.355 | 0.172 | 0.149 | 4.955E−74 | SQSTM1 | 3.72E−37 | 0.484 | 0.775 | 0.351 | 3.629E−33 |
| MT-ND1 | 8.19E−78 | −1.636 | 0.846 | 0.741 | 7.980E−74 | EPRS | 4.52E−37 | 0.387 | 0.746 | 0.333 | 4.407E−33 |
| RPL39 | 9.05E−78 | −0.902 | 0.939 | 0.636 | 8.821E−74 | SRSF3 | 6.86E−37 | 0.843 | 0.808 | 0.395 | 6.683E−33 |
| RPL26 | 1.23E−77 | −1.189 | 0.728 | 0.377 | 1.196E−73 | PRPF40A | 9.55E−37 | 0.324 | 0.613 | 0.211 | 9.308E−33 |
| RPL7A | 9.81E−76 | −0.880 | 0.844 | 0.478 | 9.562E−72 | ARPC5L | 2.46E−36 | 0.484 | 0.673 | 0.254 | 2.393E−32 |
| HMGN2 | 3.12E−75 | −1.132 | 0.791 | 0.421 | 3.044E−71 | LARS | 2.56E−36 | 0.469 | 0.622 | 0.211 | 2.496E−32 |
| RPL36 | 7.83E−75 | −1.149 | 0.853 | 0.623 | 7.633E−71 | ACTL6A | 3.04E−36 | 1.159 | 0.595 | 0.167 | 2.967E−32 |
| RN7SK | 5.27E−74 | −2.105 | 0.366 | 0.311 | 5.135E−70 | MAPKAPK2 | 3.71E−36 | 0.265 | 0.641 | 0.215 | 3.612E−32 |
| ST13 | 7.63E−74 | −0.693 | 0.761 | 0.346 | 7.431E−70 | BTG3 | 5.10E−36 | 0.325 | 0.59 | 0.206 | 4.975E−32 |
| RNA18S5 | 8.29E−74 | −2.040 | 0.228 | 0.167 | 8.080E−70 | TRAM1 | 5.56E−36 | 0.744 | 0.716 | 0.276 | 5.419E−32 |
| OAZ1 | 7.60E−73 | −1.173 | 0.855 | 0.649 | 7.406E−69 | ARMCX3 | 6.61E−36 | 0.344 | 0.719 | 0.355 | 6.441E−32 |
| MT-ATP6 | 1.88E−72 | −1.501 | 0.956 | 0.763 | 1.831E−68 | HSPH1 | 7.37E−36 | 0.310 | 0.703 | 0.311 | 7.185E−32 |
| NACA2 | 3.09E−70 | −2.019 | 0.191 | 0.132 | 3.007E−66 | DNAJC3 | 7.76E−36 | 0.344 | 0.629 | 0.263 | 7.559E−32 |
| RPL10A | 5.35E−70 | −0.444 | 0.906 | 0.482 | 5.210E−66 | ACTR3 | 7.78E−36 | 0.589 | 0.702 | 0.294 | 7.585E−32 |
| RPS9 | 2.65E−69 | −1.053 | 0.762 | 0.452 | 2.578E−65 | MRPS15 | 8.44E−36 | 0.591 | 0.673 | 0.25 | 8.223E−32 |
| SUMO2 | 9.65E−69 | −0.827 | 0.716 | 0.325 | 9.404E−65 | IRAK4 | 9.35E−36 | 0.868 | 0.625 | 0.193 | 9.114E−32 |
| SNHG5 | 4.89E−68 | −0.662 | 0.79 | 0.329 | 4.761E−64 | C17orf76- | 1.21E−35 | 0.541 | 0.71 | 0.285 | 1.182E−31 |
| | | | | | | AS1 | | | | | |
| RPS2 | 2.89E−67 | −0.719 | 0.778 | 0.364 | 2.813E−63 | MPZL1 | 1.22E−35 | 0.781 | 0.79 | 0.377 | 1.188E−31 |
| PET100 | 4.81E−67 | −1.640 | 0.463 | 0.215 | 4.686E−63 | MPC2 | 1.33E−35 | 0.738 | 0.616 | 0.206 | 1.296E−31 |
| ALDOA | 5.21E−67 | −0.886 | 0.835 | 0.522 | 5.079E−63 | PPP1R1B | 1.36E−35 | 0.617 | 0.62 | 0.215 | 1.328E−31 |
| EEF1B2 | 1.42E−66 | −0.664 | 0.75 | 0.325 | 1.382E−62 | SNRNP25 | 2.49E−35 | 0.375 | 0.445 | 0.079 | 2.422E−31 |
| CHCHD2 | 2.20E−66 | −0.920 | 0.74 | 0.386 | 2.142E−62 | DST | 2.61E−35 | 0.274 | 0.669 | 0.276 | 2.547E−31 |
| IFITM3 | 4.33E−66 | −1.142 | 0.864 | 0.689 | 4.220E−62 | C1orf27 | 3.14E−35 | 0.784 | 0.514 | 0.101 | 3.063E−31 |
| RPL37 | 4.64E−66 | −0.514 | 0.915 | 0.553 | 4.523E−62 | MRPL14 | 3.20E−35 | 0.272 | 0.614 | 0.246 | 3.115E−31 |
| EIF2S2 | 5.33E−66 | −1.569 | 0.57 | 0.338 | 5.193E−62 | PLSCR1 | 3.55E−35 | 0.357 | 0.75 | 0.377 | 3.457E−31 |
| IFITM1 | 1.90E−65 | −1.961 | 0.411 | 0.39 | 1.848E−61 | HSPA14 | 3.80E−35 | 0.625 | 0.537 | 0.14 | 3.702E−31 |
| LGALS3BP | 3.32E−65 | −0.826 | 0.775 | 0.478 | 3.239E−61 | VAMP8 | 4.92E−35 | 0.676 | 0.85 | 0.434 | 4.797E−31 |
| RPS20 | 5.01E−65 | −0.638 | 0.815 | 0.39 | 4.887E−61 | ITM2B | 5.02E−35 | 0.613 | 0.855 | 0.482 | 4.893E−31 |
| NDUFA3 | 1.56E−64 | −1.206 | 0.7 | 0.461 | 1.523E−60 | TMEM123 | 6.03E−35 | 0.539 | 0.676 | 0.289 | 5.879E−31 |
| MT-CO1 | 1.98E−64 | −1.528 | 0.958 | 0.917 | 1.930E−60 | PPIB | 6.30E−35 | 0.268 | 0.782 | 0.421 | 6.142E−31 |
| RBMX | 3.82E−64 | −0.515 | 0.766 | 0.316 | 3.727E−60 | TPM1 | 6.68E−35 | 0.366 | 0.808 | 0.443 | 6.513E−31 |
| HMGN1 | 1.32E−63 | −0.809 | 0.849 | 0.553 | 1.285E−59 | SNX3 | 7.83E−35 | 0.317 | 0.611 | 0.219 | 7.633E−31 |
| S100A1 | 1.45E−63 | −0.814 | 0.747 | 0.434 | 1.414E−59 | TM4SF1 | 8.43E−35 | 0.559 | 0.936 | 0.632 | 8.216E−31 |
| NME1- | 1.64E−63 | −1.905 | 0.272 | 0.202 | 1.601E−59 | LGALS3 | 8.50E−35 | 0.942 | 0.823 | 0.412 | 8.287E−31 |
| NME2 | | | | | | | | | | | |
| RPS15A | 2.22E−63 | −1.022 | 0.924 | 0.693 | 2.163E−59 | GDI2 | 8.75E−35 | 0.928 | 0.815 | 0.395 | 8.528E−31 |
| NDUFB11 | 1.46E−62 | −1.140 | 0.663 | 0.373 | 1.426E−58 | ESD | 1.00E−34 | 0.595 | 0.548 | 0.14 | 9.787E−31 |
| HSPB1 | 1.69E−62 | −0.868 | 0.697 | 0.338 | 1.650E−58 | PPM1G | 1.04E−34 | 0.576 | 0.778 | 0.338 | 1.009E−30 |
| NDUFA13 | 1.80E−62 | −0.873 | 0.752 | 0.404 | 1.757E−58 | OGFRL1 | 1.16E−34 | 0.321 | 0.616 | 0.232 | 1.131E−30 |
| TBCA | 3.71E−62 | −0.757 | 0.719 | 0.303 | 3.611E−58 | HEBP2 | 1.27E−34 | 1.141 | 0.902 | 0.553 | 1.239E−30 |
| RPS23 | 1.76E−61 | −0.805 | 0.944 | 0.697 | 1.712E−57 | CD46 | 1.36E−34 | 0.748 | 0.838 | 0.421 | 1.322E−30 |
| SPCS2 | 2.38E−61 | −1.695 | 0.348 | 0.184 | 2.317E−57 | PDCD10 | 1.37E−34 | 0.482 | 0.812 | 0.439 | 1.338E−30 |
| COX6A1 | 2.74E−61 | −1.060 | 0.856 | 0.675 | 2.671E−57 | NASP | 1.52E−34 | 0.626 | 0.64 | 0.219 | 1.483E−30 |
| SET | 3.76E−61 | −0.810 | 0.862 | 0.539 | 3.661E−57 | TM9SF3 | 1.59E−34 | 0.615 | 0.643 | 0.241 | 1.553E−30 |
| ATP5J2 | 3.96E−61 | −0.886 | 0.746 | 0.482 | 3.859E−57 | GALNT1 | 1.94E−34 | 0.495 | 0.533 | 0.127 | 1.893E−30 |
| GPX1 | 1.05E−60 | −1.656 | 0.501 | 0.368 | 1.025E−56 | MRAS | 2.18E−34 | 0.272 | 0.66 | 0.272 | 2.128E−30 |
| FTL | 1.94E−60 | −1.213 | 0.893 | 0.789 | 1.887E−56 | MED4 | 2.44E−34 | 0.384 | 0.534 | 0.154 | 2.379E−30 |
| SYNGR2 | 4.14E−60 | −0.910 | 0.728 | 0.399 | 4.034E−56 | MAL2 | 2.47E−34 | 0.409 | 0.676 | 0.289 | 2.410E−30 |
| RNA28S5 | 1.49E−59 | −0.304 | 0.941 | 0.741 | 1.456E−55 | MLEC | 3.48E−34 | 0.502 | 0.59 | 0.189 | 3.388E−30 |
| PTMA | 3.64E−59 | −1.068 | 0.995 | 0.93 | 3.542E−55 | ATG3 | 3.53E−34 | 0.496 | 0.67 | 0.268 | 3.444E−30 |
| CFL1 | 5.00E−59 | −0.754 | 0.858 | 0.583 | 4.871E−55 | CAPRIN1 | 3.75E−34 | 0.367 | 0.652 | 0.241 | 3.650E−30 |
| TCEB2 | 5.41E−59 | −1.235 | 0.749 | 0.544 | 5.274E−55 | HSD17B12 | 3.82E−34 | 0.371 | 0.888 | 0.553 | 3.721E−30 |
| HNRNPA1 | 9.28E−59 | −1.009 | 0.487 | 0.18 | 9.040E−55 | CLTC | 3.88E−34 | 0.335 | 0.717 | 0.342 | 3.777E−30 |
| CYBA | 1.27E−58 | −0.752 | 0.667 | 0.272 | 1.241E−54 | UBE2K | 4.01E−34 | 0.406 | 0.672 | 0.254 | 3.913E−30 |
| SNRPE | 1.33E−58 | −0.563 | 0.809 | 0.447 | 1.301E−54 | RRP15 | 4.05E−34 | 0.335 | 0.567 | 0.193 | 3.944E−30 |
| COX16 | 2.45E−58 | −0.731 | 0.644 | 0.276 | 2.383E−54 | RAD21 | 4.28E−34 | 0.268 | 0.865 | 0.557 | 4.166E−30 |
| BTF3 | 3.67E−58 | −0.408 | 0.85 | 0.425 | 3.580E−54 | SLC25A3 | 5.70E−34 | 0.756 | 0.573 | 0.162 | 5.555E−30 |
| C9orf16 | 7.19E−58 | −1.232 | 0.478 | 0.175 | 7.006E−54 | SSRP1 | 6.36E−34 | 0.336 | 0.602 | 0.232 | 6.199E−30 |
| RPS27A | 7.95E−58 | −1.101 | 0.958 | 0.82 | 7.752E−54 | CNBP | 7.14E−34 | 0.569 | 0.669 | 0.25 | 6.958E−30 |
| LGALS1 | 3.93E−57 | −1.204 | 0.885 | 0.882 | 3.826E−53 | H3F3B | 8.12E−34 | 0.312 | 0.943 | 0.697 | 7.910E−30 |
| RNY1 | 1.07E−56 | −1.294 | 0.33 | 0.123 | 1.038E−52 | MPHOSPH6 | 9.18E−34 | 0.300 | 0.617 | 0.254 | 8.943E−30 |
| EEF1D | 2.61E−56 | −1.046 | 0.654 | 0.373 | 2.541E−52 | SNRPD3 | 9.28E−34 | 0.417 | 0.604 | 0.206 | 9.039E−30 |
| SRP9 | 4.49E−56 | −0.820 | 0.743 | 0.43 | 4.378E−52 | CD164 | 9.35E−34 | 0.520 | 0.738 | 0.333 | 9.108E−30 |
| MT-ND4L | 6.93E−56 | −1.305 | 0.806 | 0.649 | 6.756E−52 | RPL30 | 1.24E−33 | 0.896 | 0.994 | 0.925 | 1.209E−29 |
| FADS2 | 1.34E−55 | −0.527 | 0.679 | 0.311 | 1.306E−51 | ABI1 | 1.53E−33 | 1.000 | 0.605 | 0.215 | 1.494E−29 |
| CD82 | 2.06E−55 | −0.751 | 0.666 | 0.342 | 2.004E−51 | HDLBP | 2.56E−33 | 0.339 | 0.887 | 0.518 | 2.495E−29 |
| CYCS | 3.35E−55 | −0.602 | 0.837 | 0.526 | 3.269E−51 | SLMO2 | 2.77E−33 | 0.804 | 0.602 | 0.18 | 2.700E−29 |
| TIMM13 | 3.36E−55 | −0.967 | 0.545 | 0.263 | 3.273E−51 | FAM213A | 3.04E−33 | 0.510 | 0.772 | 0.377 | 2.963E−29 |
| NDUFA11 | 1.15E−54 | −0.664 | 0.702 | 0.342 | 1.120E−50 | DPY30 | 3.21E−33 | 0.277 | 0.531 | 0.154 | 3.132E−29 |
| DNAJC19 | 1.23E−54 | −0.517 | 0.675 | 0.263 | 1.196E−50 | MORF4L2 | 3.31E−33 | 0.316 | 0.652 | 0.254 | 3.227E−29 |
| COX7B | 1.27E−54 | −0.900 | 0.693 | 0.377 | 1.241E−50 | ILF3 | 3.47E−33 | 0.499 | 0.555 | 0.171 | 3.379E−29 |
| USMG5 | 1.60E−54 | −0.695 | 0.79 | 0.474 | 1.559E−50 | PAPOLA | 3.53E−33 | 0.326 | 0.746 | 0.382 | 3.441E−29 |
| HLA-A | 3.25E−54 | −1.088 | 0.693 | 0.522 | 3.163E−50 | NET1 | 3.69E−33 | 0.573 | 0.56 | 0.184 | 3.595E−29 |
| CTD- | 4.49E−54 | −1.928 | 0.132 | 0.083 | 4.375E−50 | BZW2 | 3.79E−33 | 0.308 | 0.584 | 0.206 | 3.694E−29 |
| 2090I13.1 | | | | | | | | | | | |
| ATP5I | 4.67E−54 | −0.960 | 0.563 | 0.294 | 4.546E−50 | BEX2 | 4.53E−33 | 0.741 | 0.539 | 0.149 | 4.413E−29 |
| UQCR10 | 5.11E−54 | −0.756 | 0.694 | 0.386 | 4.984E−50 | ATP6V1G1 | 5.22E−33 | 0.764 | 0.82 | 0.465 | 5.082E−29 |
| HSPE1 | 1.28E−53 | −0.591 | 0.705 | 0.368 | 1.250E−49 | ITGA6 | 6.23E−33 | 0.366 | 0.522 | 0.167 | 6.069E−29 |
| UBA52 | 1.49E−53 | −0.439 | 0.868 | 0.518 | 1.450E−49 | YME1L1 | 6.34E−33 | 0.286 | 0.64 | 0.285 | 6.178E−29 |
| KRT8 | 1.85E−53 | −0.698 | 0.667 | 0.32 | 1.807E−49 | RNF7 | 7.45E−33 | 0.637 | 0.599 | 0.197 | 7.262E−29 |
| HLA-C | 2.23E−53 | −0.785 | 0.837 | 0.618 | 2.169E−49 | RN7SL1 | 7.94E−33 | 0.730 | 0.381 | 0.035 | 7.739E−29 |
| CDC42 | 3.58E−53 | −0.304 | 0.725 | 0.316 | 3.493E−49 | COPS2 | 8.25E−33 | 0.332 | 0.596 | 0.197 | 8.040E−29 |
| CD74 | 4.23E−53 | −1.642 | 0.495 | 0.443 | 4.117E−49 | GLTSCR2 | 9.37E−33 | 0.268 | 0.492 | 0.114 | 9.130E−29 |
| CUTA | 8.42E−53 | −0.852 | 0.611 | 0.281 | 8.204E−49 | PSME4 | 9.42E−33 | 0.444 | 0.631 | 0.263 | 9.176E−29 |
| MRPL36 | 1.08E−52 | −0.508 | 0.806 | 0.478 | 1.050E−48 | PTP4A1 | 1.00E−32 | 0.402 | 0.607 | 0.232 | 9.742E−29 |
| PRDX2 | 1.12E−52 | −1.065 | 0.457 | 0.224 | 1.087E−48 | CCT3 | 1.00E−32 | 0.627 | 0.829 | 0.456 | 9.746E−29 |
| FXYD5 | 1.40E−52 | −1.470 | 0.437 | 0.276 | 1.363E−48 | KDELR1 | 1.01E−32 | 0.423 | 0.566 | 0.171 | 9.824E−29 |
| ATP5G2 | 1.48E−52 | −0.656 | 0.676 | 0.294 | 1.438E−48 | SCRN1 | 1.03E−32 | 0.631 | 0.602 | 0.224 | 1.007E−28 |
| PCBP2 | 1.59E−52 | −0.867 | 0.561 | 0.289 | 1.547E−48 | NAP1L1 | 1.09E−32 | 0.352 | 0.599 | 0.215 | 1.059E−28 |
| SUB1 | 3.76E−52 | −0.306 | 0.867 | 0.513 | 3.662E−48 | OCIAD1 | 1.39E−32 | 1.117 | 0.566 | 0.145 | 1.359E−28 |
| COPE | 4.56E−52 | −1.189 | 0.428 | 0.175 | 4.442E−48 | ERP29 | 1.81E−32 | 0.295 | 0.563 | 0.175 | 1.767E−28 |
| POMP | 8.79E−52 | −0.305 | 0.828 | 0.487 | 8.569E−48 | TKT | 1.90E−32 | 0.359 | 0.675 | 0.285 | 1.856E−28 |
| PRELID1 | 1.09E−51 | −0.535 | 0.713 | 0.32 | 1.059E−47 | MRPL13 | 2.35E−32 | 0.651 | 0.725 | 0.342 | 2.285E−28 |
| ATP5L | 1.26E−51 | −0.290 | 0.702 | 0.294 | 1.227E−47 | DSC2 | 2.60E−32 | 0.502 | 0.829 | 0.509 | 2.537E−28 |
| TAGLN2 | 1.54E−51 | −0.543 | 0.88 | 0.605 | 1.502E−47 | EMC4 | 2.82E−32 | 0.778 | 0.569 | 0.158 | 2.751E−28 |
| RPL36AL | 1.77E−51 | −0.891 | 0.579 | 0.303 | 1.728E−47 | PSMD8 | 2.85E−32 | 0.427 | 0.563 | 0.202 | 2.774E−28 |
| CSTB | 2.46E−51 | −0.333 | 0.814 | 0.434 | 2.400E−47 | NDUFB10 | 2.98E−32 | 0.348 | 0.626 | 0.25 | 2.907E−28 |
| HLA-B | 5.36E−51 | −0.953 | 0.894 | 0.798 | 5.226E−47 | NUCB2 | 3.47E−32 | 0.338 | 0.643 | 0.254 | 3.381E−28 |
| SERBP1 | 8.97E−51 | −0.482 | 0.658 | 0.303 | 8.739E−47 | GGCT | 4.16E−32 | 0.458 | 0.773 | 0.386 | 4.056E−28 |
| RPL17 | 1.07E−50 | −1.478 | 0.251 | 0.123 | 1.044E−46 | GLRX | 4.56E−32 | 0.585 | 0.463 | 0.101 | 4.448E−28 |
| UBL5 | 1.62E−50 | −0.253 | 0.856 | 0.461 | 1.578E−46 | EIF2AK2 | 4.70E−32 | 0.320 | 0.72 | 0.36 | 4.583E−28 |
| MORF4L1 | 3.40E−50 | −1.285 | 0.448 | 0.197 | 3.310E−46 | XBP1 | 5.53E−32 | 0.869 | 0.49 | 0.101 | 5.387E−28 |
| STRA13 | 3.40E−50 | −0.377 | 0.71 | 0.408 | 3.311E−46 | RAB4A | 6.27E−32 | 0.579 | 0.517 | 0.136 | 6.107E−28 |
| PA2G4 | 3.51E−50 | −0.503 | 0.72 | 0.351 | 3.421E−46 | GTPBP4 | 7.36E−32 | 0.338 | 0.569 | 0.211 | 7.176E−28 |
| SNRPA1 | 3.90E−50 | −0.900 | 0.593 | 0.281 | 3.801E−46 | DHX9 | 7.84E−32 | 0.476 | 0.694 | 0.276 | 7.637E−28 |
| NDUFC2 | 4.42E−50 | −1.037 | 0.558 | 0.325 | 4.307E−46 | RBBP7 | 8.31E−32 | 0.623 | 0.558 | 0.167 | 8.101E−28 |
| AURKAIP1 | 5.11E−50 | −0.770 | 0.646 | 0.32 | 4.981E−46 | VPS29 | 9.19E−32 | 0.473 | 0.569 | 0.206 | 8.957E−28 |
| PSME2 | 6.98E−50 | −1.311 | 0.508 | 0.285 | 6.802E−46 | CP | 1.10E−31 | 0.858 | 0.75 | 0.382 | 1.070E−27 |
| HMGB3 | 8.30E−50 | −1.015 | 0.567 | 0.32 | 8.086E−46 | ATF3 | 1.31E−31 | 1.320 | 0.519 | 0.127 | 1.277E−27 |
| CDKN2A | 1.09E−49 | −0.361 | 0.64 | 0.232 | 1.064E−45 | USP39 | 1.44E−31 | 0.393 | 0.484 | 0.136 | 1.408E−27 |
| RPS4X | 1.61E−49 | −0.593 | 0.847 | 0.557 | 1.569E−45 | PSMD2 | 1.63E−31 | 0.300 | 0.595 | 0.224 | 1.585E−27 |
| TOMM7 | 2.60E−49 | −0.347 | 0.811 | 0.443 | 2.529E−45 | NARF | 1.86E−31 | 0.401 | 0.481 | 0.132 | 1.816E−27 |
| HNRNPC | 3.04E−49 | −0.347 | 0.799 | 0.377 | 2.964E−45 | COMMD2 | 1.89E−31 | 0.401 | 0.598 | 0.219 | 1.843E−27 |
| MINOS1 | 3.37E−49 | −1.327 | 0.424 | 0.202 | 3.284E−45 | UBXN2A | 2.11E−31 | 0.395 | 0.415 | 0.075 | 2.055E−27 |
| TUBA1B | 3.67E−49 | −0.772 | 0.759 | 0.478 | 3.572E−45 | RBM39 | 2.11E−31 | 0.272 | 0.567 | 0.206 | 2.057E−27 |
| UBC | 3.92E−49 | −0.783 | 0.681 | 0.399 | 3.820E−45 | SF3B14 | 2.21E−31 | 0.388 | 0.651 | 0.289 | 2.157E−27 |
| ENSA | 6.24E−49 | −0.325 | 0.629 | 0.285 | 6.083E−45 | HNRNPAB | 2.30E−31 | 0.338 | 0.554 | 0.189 | 2.241E−27 |
| NME1 | 9.38E−49 | −0.803 | 0.651 | 0.346 | 9.138E−45 | IARS2 | 2.41E−31 | 0.490 | 0.785 | 0.412 | 2.347E−27 |
| VDAC2 | 1.33E−48 | −1.426 | 0.395 | 0.202 | 1.291E−44 | PLOD2 | 2.45E−31 | 0.550 | 0.511 | 0.136 | 2.390E−27 |
| POLR2L | 2.20E−48 | −0.367 | 0.775 | 0.421 | 2.142E−44 | ATP1A1 | 3.02E−31 | 0.453 | 0.593 | 0.224 | 2.946E−27 |
| RPL32 | 2.94E−48 | −0.948 | 0.962 | 0.829 | 2.862E−44 | MAP1LC3B | 3.09E−31 | 0.408 | 0.573 | 0.206 | 3.011E−27 |
| DYNLL1 | 3.64E−48 | −0.681 | 0.756 | 0.491 | 3.549E−44 | BROX | 3.20E−31 | 0.375 | 0.738 | 0.346 | 3.114E−27 |
| SSB | 6.36E−48 | −0.778 | 0.558 | 0.268 | 6.200E−44 | PDXK | 3.27E−31 | 0.666 | 0.587 | 0.202 | 3.190E−27 |
| YBX1 | 9.15E−48 | −0.818 | 0.941 | 0.763 | 8.913E−44 | MED10 | 3.29E−31 | 0.456 | 0.602 | 0.232 | 3.205E−27 |
| SNRPC | 1.79E−47 | −0.321 | 0.679 | 0.281 | 1.743E−43 | SF3B1 | 3.40E−31 | 0.528 | 0.625 | 0.254 | 3.317E−27 |
| FSCN1 | 2.34E−47 | −0.351 | 0.516 | 0.145 | 2.276E−43 | GNB2L1 | 3.99E−31 | 0.301 | 0.921 | 0.618 | 3.884E−27 |
| ANXA2 | 2.85E−47 | −0.661 | 0.847 | 0.601 | 2.773E−43 | RGS2 | 4.25E−31 | 0.696 | 0.585 | 0.224 | 4.143E−27 |
| C19orf53 | 3.01E−47 | −0.553 | 0.596 | 0.259 | 2.931E−43 | CYB5A | 4.45E−31 | 0.671 | 0.477 | 0.114 | 4.334E−27 |
| ATP5B | 6.62E−47 | −0.741 | 0.599 | 0.285 | 6.450E−43 | ACP1 | 4.57E−31 | 0.415 | 0.672 | 0.298 | 4.453E−27 |
| PRDX5 | 7.71E−47 | −0.952 | 0.548 | 0.281 | 7.509E−43 | EIF2A | 4.90E−31 | 0.906 | 0.56 | 0.154 | 4.777E−27 |
| COX20 | 8.70E−47 | −0.281 | 0.657 | 0.263 | 8.475E−43 | PGRMC1 | 5.66E−31 | 0.567 | 0.752 | 0.36 | 5.511E−27 |
| SEPW1 | 1.12E−46 | −0.441 | 0.697 | 0.342 | 1.089E−42 | UFM1 | 5.98E−31 | 0.458 | 0.613 | 0.228 | 5.825E−27 |
| MTRNR2L10 | 1.12E−46 | −1.627 | 0.188 | 0.123 | 1.090E−42 | GLRX2 | 6.56E−31 | 0.560 | 0.548 | 0.162 | 6.397E−27 |
| GSTP1 | 1.75E−46 | −0.366 | 0.849 | 0.605 | 1.706E−42 | TMEM50A | 6.62E−31 | 0.259 | 0.486 | 0.145 | 6.455E−27 |
| TNFRSF12A | 1.83E−46 | −1.022 | 0.563 | 0.355 | 1.787E−42 | NAA50 | 1.08E−30 | 0.442 | 0.522 | 0.149 | 1.052E−26 |
| ROMO1 | 1.87E−46 | −0.292 | 0.759 | 0.386 | 1.824E−42 | HSPA5 | 1.10E−30 | 0.428 | 0.831 | 0.522 | 1.077E−26 |
| CBWD1 | 2.64E−46 | −0.434 | 0.528 | 0.149 | 2.569E−42 | TMCO3 | 1.26E−30 | 0.791 | 0.502 | 0.127 | 1.228E−26 |
| TPI1 | 2.93E−46 | −0.649 | 0.796 | 0.553 | 2.855E−42 | XRCC5 | 1.27E−30 | 0.368 | 0.623 | 0.232 | 1.240E−26 |
| ACTG1 | 3.19E−46 | −0.401 | 0.936 | 0.746 | 3.113E−42 | DDX5 | 1.29E−30 | 0.389 | 0.865 | 0.518 | 1.253E−26 |
| CETN2 | 4.46E−46 | −0.272 | 0.678 | 0.281 | 4.350E−42 | UBE2N | 1.31E−30 | 0.463 | 0.533 | 0.167 | 1.281E−26 |
| SNHG9 | 5.71E−46 | −1.044 | 0.343 | 0.088 | 5.566E−42 | COPB1 | 1.37E−30 | 0.377 | 0.566 | 0.184 | 1.333E−26 |
| NDUFB4 | 8.23E−46 | −0.668 | 0.623 | 0.281 | 8.016E−42 | ACBD3 | 1.54E−30 | 0.334 | 0.545 | 0.184 | 1.501E−26 |
| RPL15 | 1.01E−45 | −0.811 | 0.956 | 0.829 | 9.882E−42 | MRPS34 | 1.56E−30 | 0.333 | 0.661 | 0.316 | 1.518E−26 |
| RPL18 | 1.80E−45 | −0.343 | 0.835 | 0.513 | 1.758E−41 | CANX | 1.61E−30 | 0.630 | 0.95 | 0.68 | 1.566E−26 |
| PTMS | 2.14E−45 | −0.317 | 0.452 | 0.101 | 2.087E−41 | FBXO9 | 1.66E−30 | 0.362 | 0.576 | 0.211 | 1.619E−26 |
| TCEAL8 | 2.42E−45 | −0.254 | 0.732 | 0.329 | 2.359E−41 | ENO1 | 1.68E−30 | 0.699 | 0.959 | 0.724 | 1.637E−26 |
| RANBP1 | 3.53E−45 | −0.368 | 0.643 | 0.246 | 3.440E−41 | HTATSF1 | 2.09E−30 | 0.300 | 0.507 | 0.175 | 2.039E−26 |
| PCNP | 4.34E−45 | −0.461 | 0.685 | 0.329 | 4.225E−41 | DSTN | 2.42E−30 | 0.427 | 0.939 | 0.623 | 2.360E−26 |
| TMEM176B | 4.53E−45 | −0.272 | 0.644 | 0.246 | 4.417E−41 | YES1 | 2.74E−30 | 0.333 | 0.56 | 0.184 | 2.673E−26 |
| RPS19 | 6.04E−45 | −0.948 | 0.92 | 0.829 | 5.886E−41 | NDUFA10 | 2.84E−30 | 0.482 | 0.542 | 0.171 | 2.768E−26 |
| SLC25A6 | 6.99E−45 | −0.442 | 0.814 | 0.531 | 6.812E−41 | DUT | 3.46E−30 | 1.071 | 0.554 | 0.162 | 3.372E−26 |
| CARHSP1 | 8.17E−45 | −0.312 | 0.57 | 0.18 | 7.966E−41 | SAP30BP | 3.47E−30 | 0.423 | 0.487 | 0.149 | 3.385E−26 |
| MIR4458HG | 8.44E−45 | −1.294 | 0.407 | 0.193 | 8.222E−41 | FKBP3 | 3.60E−30 | 0.354 | 0.641 | 0.281 | 3.512E−26 |
| 7-Sep | 8.87E−45 | −0.383 | 0.776 | 0.412 | 8.644E−41 | SMARCC1 | 3.76E−30 | 0.721 | 0.489 | 0.11 | 3.666E−26 |
| RPLP0 | 9.80E−45 | −0.756 | 0.958 | 0.759 | 9.551E−41 | PSMA3 | 4.07E−30 | 0.683 | 0.552 | 0.18 | 3.969E−26 |
| EIF2S3L | 2.12E−44 | −1.012 | 0.183 | 0.048 | 2.065E−40 | PRDX1 | 4.09E−30 | 0.333 | 0.944 | 0.697 | 3.989E−26 |
| PSMB5 | 3.77E−44 | −0.502 | 0.64 | 0.333 | 3.669E−40 | CFI | 4.12E−30 | 0.438 | 0.705 | 0.39 | 4.020E−26 |
| CBX3 | 4.16E−44 | −0.488 | 0.938 | 0.711 | 4.054E−40 | RAB10 | 5.15E−30 | 0.491 | 0.67 | 0.281 | 5.017E−26 |
| SDHC | 4.20E−44 | −1.332 | 0.316 | 0.145 | 4.089E−40 | SRSF5 | 6.06E−30 | 0.404 | 0.514 | 0.136 | 5.907E−26 |
| TWF1 | 4.42E−44 | −0.423 | 0.764 | 0.434 | 4.304E−40 | EIF3M | 6.50E−30 | 0.554 | 0.61 | 0.232 | 6.338E−26 |
| COX6C | 7.36E−44 | −0.637 | 0.599 | 0.364 | 7.169E−40 | GTF2A2 | 7.79E−30 | 0.494 | 0.534 | 0.189 | 7.587E−26 |
| ARPC1B | 9.66E−44 | −0.603 | 0.638 | 0.298 | 9.413E−40 | TP53BP2 | 8.16E−30 | 0.397 | 0.523 | 0.18 | 7.952E−26 |
| UQCRFS1 | 1.06E−43 | −1.069 | 0.43 | 0.202 | 1.038E−39 | CCT6A | 8.42E−30 | 0.509 | 0.716 | 0.377 | 8.207E−26 |
| ZNHIT1 | 1.15E−43 | −0.695 | 0.566 | 0.232 | 1.119E−39 | CINP | 8.50E−30 | 0.661 | 0.461 | 0.101 | 8.280E−26 |
| AK2 | 1.23E−43 | −0.251 | 0.542 | 0.167 | 1.196E−39 | YY1 | 9.57E−30 | 0.259 | 0.519 | 0.18 | 9.330E−26 |
| APRT | 1.35E−43 | −0.757 | 0.564 | 0.285 | 1.319E−39 | ART3 | 9.74E−30 | 0.331 | 0.658 | 0.307 | 9.491E−26 |
| CBX1 | 1.41E−43 | −0.457 | 0.584 | 0.228 | 1.377E−39 | UBE2J1 | 1.01E−29 | 0.554 | 0.477 | 0.123 | 9.863E−26 |
| DYNC1I2 | 2.75E−43 | −0.929 | 0.401 | 0.14 | 2.683E−39 | RPL22L1 | 1.31E−29 | 0.308 | 0.407 | 0.083 | 1.273E−25 |
| EIF4B | 3.01E−43 | −0.485 | 0.404 | 0.092 | 2.934E−39 | ELOVL6 | 1.49E−29 | 0.311 | 0.681 | 0.307 | 1.448E−25 |
| RBBP4 | 3.78E−43 | −0.354 | 0.555 | 0.193 | 3.684E−39 | GSPT1 | 1.54E−29 | 0.422 | 0.543 | 0.167 | 1.497E−25 |
| ARHGDIA | 3.80E−43 | −0.722 | 0.554 | 0.311 | 3.700E−39 | GMPS | 1.66E−29 | 0.751 | 0.534 | 0.149 | 1.613E−25 |
| ISOC2 | 3.84E−43 | −1.422 | 0.352 | 0.154 | 3.739E−39 | CMSS1 | 1.68E−29 | 0.348 | 0.505 | 0.154 | 1.641E−25 |
| SLC25A39 | 4.27E−43 | −1.021 | 0.445 | 0.211 | 4.157E−39 | HADHA | 1.84E−29 | 0.326 | 0.694 | 0.36 | 1.792E−25 |
| ZC3H11A | 4.35E−43 | −0.553 | 0.563 | 0.237 | 4.242E−39 | PAPSS1 | 1.86E−29 | 0.452 | 0.607 | 0.276 | 1.811E−25 |
| RHEB | 5.81E−43 | −1.371 | 0.387 | 0.189 | 5.664E−39 | SOX4 | 2.10E−29 | 0.584 | 0.688 | 0.346 | 2.046E−25 |
| SNRPB | 6.10E−43 | −0.557 | 0.495 | 0.171 | 5.947E−39 | SENP6 | 2.35E−29 | 0.554 | 0.52 | 0.154 | 2.286E−25 |
| S100A13 | 7.83E−43 | −0.304 | 0.657 | 0.276 | 7.630E−39 | STARD7 | 2.40E−29 | 0.251 | 0.513 | 0.175 | 2.341E−25 |
| RCN1 | 8.39E−43 | −0.697 | 0.634 | 0.351 | 8.173E−39 | YWHAH | 2.84E−29 | 0.538 | 0.486 | 0.14 | 2.767E−25 |
| OAT | 9.34E−43 | −0.425 | 0.744 | 0.421 | 9.105E−39 | PYURF | 2.98E−29 | 0.301 | 0.722 | 0.36 | 2.908E−25 |
| PEBP1 | 9.73E−43 | −0.428 | 0.834 | 0.5 | 9.482E−39 | VIM | 3.01E−29 | 0.297 | 0.85 | 0.522 | 2.934E−25 |
| CTD- | 1.15E−42 | −1.728 | 0.082 | 0.11 | 1.124E−38 | PAIP2 | 4.18E−29 | 0.317 | 0.548 | 0.197 | 4.076E−25 |
| 2192J16.22 | | | | | | | | | | | |
| RBM8A | 1.21E−42 | −0.831 | 0.439 | 0.197 | 1.183E−38 | BBOX1 | 4.25E−29 | 0.904 | 0.557 | 0.171 | 4.144E−25 |
| BZW1 | 1.31E−42 | −1.053 | 0.486 | 0.25 | 1.274E−38 | AP2M1 | 4.63E−29 | 0.380 | 0.589 | 0.228 | 4.509E−25 |
| SNRPD1 | 1.48E−42 | −0.581 | 0.616 | 0.32 | 1.443E−38 | SRP72 | 4.76E−29 | 0.465 | 0.649 | 0.272 | 4.637E−25 |
| RPS27L | 1.73E−42 | −0.561 | 0.564 | 0.224 | 1.686E−38 | COPZ1 | 5.30E−29 | 0.561 | 0.575 | 0.202 | 5.168E−25 |
| EIF3I | 1.86E−42 | −0.336 | 0.707 | 0.351 | 1.810E−38 | VMP1 | 5.77E−29 | 0.303 | 0.534 | 0.206 | 5.623E−25 |
| HAX1 | 2.67E−42 | −0.305 | 0.557 | 0.193 | 2.600E−38 | RAB2A | 6.72E−29 | 0.285 | 0.436 | 0.114 | 6.546E−25 |
| SLIRP | 2.70E−42 | −0.901 | 0.652 | 0.43 | 2.634E−38 | EIF4A2 | 9.01E−29 | 0.332 | 0.475 | 0.127 | 8.783E−25 |
| RPL7L1 | 2.92E−42 | −1.102 | 0.383 | 0.162 | 2.841E−38 | KDSR | 9.93E−29 | 0.906 | 0.39 | 0.053 | 9.681E−25 |
| AP2S1 | 3.12E−42 | −0.692 | 0.519 | 0.25 | 3.040E−38 | CCT2 | 1.03E−28 | 0.960 | 0.52 | 0.14 | 1.004E−24 |
| NHP2 | 3.13E−42 | −0.400 | 0.766 | 0.417 | 3.046E−38 | CXADR | 1.07E−28 | 0.357 | 0.542 | 0.202 | 1.042E−24 |
| NDUFB1 | 3.29E−42 | −0.307 | 0.551 | 0.189 | 3.203E−38 | IFT57 | 1.13E−28 | 0.718 | 0.738 | 0.36 | 1.104E−24 |
| LSM3 | 3.46E−42 | −0.381 | 0.652 | 0.346 | 3.370E−38 | CREG1 | 1.20E−28 | 0.498 | 0.543 | 0.189 | 1.165E−24 |
| HIST1H2AM | 3.68E−42 | −2.063 | 0.123 | 0.123 | 3.585E−38 | CALU | 1.24E−28 | 0.534 | 0.601 | 0.224 | 1.205E−24 |
| ZNF706 | 4.01E−42 | −0.434 | 0.604 | 0.316 | 3.905E−38 | DHRS7 | 1.42E−28 | 0.255 | 0.67 | 0.316 | 1.380E−24 |
| HMGN3 | 4.69E−42 | −0.494 | 0.554 | 0.228 | 4.571E−38 | MOB1A | 1.55E−28 | 0.346 | 0.593 | 0.241 | 1.507E−24 |
| CYP51A1 | 4.78E−42 | −0.798 | 0.474 | 0.193 | 4.658E−38 | CDC5L | 1.57E−28 | 0.384 | 0.475 | 0.145 | 1.533E−24 |
| TAPBP | 7.22E−42 | −0.543 | 0.664 | 0.351 | 7.034E−38 | NRD1 | 1.66E−28 | 0.378 | 0.548 | 0.206 | 1.615E−24 |
| TUBB | 8.54E−42 | −0.638 | 0.585 | 0.298 | 8.324E−38 | TIAL1 | 1.86E−28 | 0.305 | 0.487 | 0.14 | 1.816E−24 |
| LHCGR | 1.11E−41 | −0.646 | 0.212 | 0.026 | 1.078E−37 | API5 | 1.86E−28 | 0.291 | 0.446 | 0.132 | 1.817E−24 |
| SH3BGRL3 | 1.47E−41 | −1.297 | 0.416 | 0.237 | 1.436E−37 | SPTBN1 | 1.89E−28 | 0.360 | 0.579 | 0.241 | 1.844E−24 |
| APH1A | 1.65E−41 | −0.674 | 0.47 | 0.149 | 1.607E−37 | TNFSF10 | 1.89E−28 | 0.508 | 0.601 | 0.268 | 1.846E−24 |
| MT-ND5 | 1.69E−41 | −0.728 | 0.817 | 0.623 | 1.643E−37 | RARRES1 | 2.11E−28 | 1.410 | 0.888 | 0.763 | 2.057E−24 |
| ARPC3 | 1.74E−41 | −0.434 | 0.604 | 0.263 | 1.700E−37 | MCUR1 | 2.98E−28 | 0.451 | 0.499 | 0.158 | 2.899E−24 |
| TALDO1 | 1.87E−41 | −0.291 | 0.596 | 0.25 | 1.822E−37 | SCD5 | 3.23E−28 | 0.338 | 0.458 | 0.127 | 3.152E−24 |
| RGS10 | 2.16E−41 | −0.273 | 0.732 | 0.368 | 2.105E−37 | SMARCA5 | 3.56E−28 | 0.352 | 0.579 | 0.228 | 3.466E−24 |
| U2AF1 | 2.77E−41 | −0.456 | 0.664 | 0.338 | 2.701E−37 | CLIC4 | 3.77E−28 | 0.485 | 0.508 | 0.162 | 3.678E−24 |
| NOP10 | 3.36E−41 | −0.805 | 0.589 | 0.32 | 3.272E−37 | MBNL2 | 4.04E−28 | 0.416 | 0.62 | 0.259 | 3.939E−24 |
| PRDX4 | 4.67E−41 | −0.643 | 0.693 | 0.421 | 4.551E−37 | INSR | 4.16E−28 | 0.301 | 0.455 | 0.14 | 4.056E−24 |
| MAGOH | 5.11E−41 | −0.652 | 0.498 | 0.202 | 4.984E−37 | PTPLAD1 | 4.21E−28 | 0.294 | 0.587 | 0.219 | 4.105E−24 |
| MT-CO3 | 5.65E−41 | −0.992 | 0.98 | 0.956 | 5.507E−37 | SEC63 | 4.34E−28 | 0.303 | 0.554 | 0.197 | 4.232E−24 |
| HNRNPA3 | 6.73E−41 | −1.190 | 0.398 | 0.206 | 6.560E−37 | MRPS35 | 4.38E−28 | 0.549 | 0.539 | 0.193 | 4.264E−24 |
| COX17 | 8.02E−41 | −0.645 | 0.451 | 0.167 | 7.819E−37 | CAMTA1 | 4.58E−28 | 0.371 | 0.469 | 0.132 | 4.467E−24 |
| HNRNPK | 8.93E−41 | −0.334 | 0.876 | 0.588 | 8.698E−37 | MX1 | 6.06E−28 | 0.354 | 0.74 | 0.443 | 5.907E−24 |
| ACTN4 | 9.04E−41 | −0.928 | 0.561 | 0.325 | 8.807E−37 | QSER1 | 6.47E−28 | 0.370 | 0.537 | 0.232 | 6.301E−24 |
| TMED9 | 1.40E−40 | −0.422 | 0.601 | 0.32 | 1.367E−36 | ZDHHC20 | 6.74E−28 | 0.537 | 0.466 | 0.118 | 6.566E−24 |
| RNF181 | 3.07E−40 | −0.618 | 0.54 | 0.232 | 2.996E−36 | PIK3R1 | 8.36E−28 | 0.528 | 0.49 | 0.14 | 8.151E−24 |
| APOD | 3.20E−40 | −0.372 | 0.77 | 0.5 | 3.120E−36 | PRPF6 | 8.36E−28 | 0.283 | 0.478 | 0.145 | 8.151E−24 |
| RAN | 3.24E−40 | −0.315 | 0.79 | 0.465 | 3.160E−36 | GLO1 | 8.72E−28 | 0.505 | 0.714 | 0.338 | 8.496E−24 |
| TMEM258 | 4.20E−40 | −0.483 | 0.62 | 0.333 | 4.096E−36 | RAB3GAP2 | 8.82E−28 | 0.272 | 0.495 | 0.18 | 8.598E−24 |
| SHFM1 | 5.80E−40 | −0.281 | 0.575 | 0.224 | 5.651E−36 | SRP14 | 9.09E−28 | 0.258 | 0.844 | 0.5 | 8.855E−24 |
| CRYAB | 7.01E−40 | −0.789 | 0.573 | 0.316 | 6.828E−36 | ACTR6 | 9.76E−28 | 1.167 | 0.56 | 0.189 | 9.512E−24 |
| LINC00152 | 7.65E−40 | −1.324 | 0.407 | 0.224 | 7.451E−36 | SH3GLB1 | 1.07E−27 | 0.267 | 0.433 | 0.136 | 1.045E−23 |
| SEC61B | 9.41E−40 | −0.380 | 0.72 | 0.408 | 9.167E−36 | POLR2B | 1.20E−27 | 0.485 | 0.51 | 0.149 | 1.173E−23 |
| RPL4 | 1.00E−39 | −0.362 | 0.893 | 0.623 | 9.774E−36 | CBX5 | 1.57E−27 | 0.325 | 0.534 | 0.241 | 1.532E−23 |
| HMGA1 | 1.27E−39 | −0.277 | 0.675 | 0.325 | 1.233E−35 | CNIH1 | 1.64E−27 | 0.859 | 0.47 | 0.123 | 1.603E−23 |
| RPL26L1 | 1.37E−39 | −0.826 | 0.434 | 0.154 | 1.333E−35 | PHF20L1 | 1.88E−27 | 0.522 | 0.502 | 0.154 | 1.836E−23 |
| SNRNP27 | 1.59E−39 | −0.356 | 0.531 | 0.149 | 1.547E−35 | ZMPSTE24 | 1.89E−27 | 0.375 | 0.59 | 0.276 | 1.841E−23 |
| NDUFA6 | 1.82E−39 | −0.377 | 0.853 | 0.548 | 1.773E−35 | SCPEP1 | 1.96E−27 | 0.451 | 0.372 | 0.057 | 1.907E−23 |
| GPX4 | 2.02E−39 | −0.383 | 0.632 | 0.316 | 1.970E−35 | C16orf91 | 1.97E−27 | 0.320 | 0.369 | 0.083 | 1.918E−23 |
| PDAP1 | 2.95E−39 | −0.341 | 0.498 | 0.162 | 2.876E−35 | SH3YL1 | 2.07E−27 | 0.809 | 0.499 | 0.14 | 2.021E−23 |
| HSD17B10 | 2.99E−39 | −0.430 | 0.483 | 0.206 | 2.912E−35 | OXR1 | 2.31E−27 | 0.379 | 0.43 | 0.127 | 2.247E−23 |
| LSMD1 | 3.32E−39 | −0.440 | 0.478 | 0.18 | 3.239E−35 | ANXA5 | 2.49E−27 | 0.378 | 0.744 | 0.404 | 2.425E−23 |
| PTPMT1 | 3.54E−39 | −0.947 | 0.333 | 0.096 | 3.447E−35 | RNF13 | 2.59E−27 | 1.484 | 0.455 | 0.092 | 2.528E−23 |
| CBWD2 | 3.72E−39 | −1.175 | 0.348 | 0.145 | 3.626E−35 | TXNDC12 | 2.87E−27 | 0.413 | 0.463 | 0.154 | 2.793E−23 |
| YWHAZ | 4.45E−39 | −0.720 | 0.958 | 0.833 | 4.334E−35 | COG2 | 2.96E−27 | 0.254 | 0.443 | 0.158 | 2.886E−23 |
| DDX21 | 4.88E−39 | −0.264 | 0.635 | 0.316 | 4.759E−35 | ASH1L | 2.97E−27 | 0.344 | 0.536 | 0.215 | 2.898E−23 |
| CAPG | 5.06E−39 | −0.586 | 0.595 | 0.303 | 4.927E−35 | NCOA4 | 3.06E−27 | 0.814 | 0.445 | 0.118 | 2.978E−23 |
| EMD | 5.19E−39 | −0.560 | 0.49 | 0.228 | 5.056E−35 | C19orf43 | 3.39E−27 | 0.327 | 0.378 | 0.092 | 3.307E−23 |
| ATOX1 | 6.01E−39 | −0.573 | 0.431 | 0.149 | 5.855E−35 | IRF2BP2 | 4.08E−27 | 0.305 | 0.572 | 0.263 | 3.978E−23 |
| GNB1 | 6.17E−39 | −0.309 | 0.678 | 0.342 | 6.017E−35 | CCDC90B | 4.72E−27 | 0.887 | 0.486 | 0.123 | 4.600E−23 |
| FLNA | 6.31E−39 | −0.929 | 0.552 | 0.395 | 6.154E−35 | ELOVL5 | 4.97E−27 | 0.609 | 0.499 | 0.158 | 4.845E−23 |
| PFDN5 | 6.49E−39 | −0.508 | 0.575 | 0.281 | 6.320E−35 | SMIM11 | 5.34E−27 | 0.276 | 0.375 | 0.075 | 5.202E−23 |
| NAA10 | 7.18E−39 | −1.124 | 0.424 | 0.184 | 7.000E−35 | SMEK2 | 5.39E−27 | 0.384 | 0.614 | 0.281 | 5.250E−23 |
| HSBP1 | 7.39E−39 | −0.259 | 0.902 | 0.684 | 7.197E−35 | MAPKAP1 | 5.98E−27 | 0.741 | 0.56 | 0.18 | 5.830E−23 |
| RPS3 | 8.80E−39 | −0.453 | 0.918 | 0.649 | 8.576E−35 | TOMM22 | 6.09E−27 | 0.517 | 0.427 | 0.101 | 5.935E−23 |
| ACTB | 9.22E−39 | −0.611 | 0.947 | 0.855 | 8.988E−35 | ARL6IP5 | 6.18E−27 | 0.403 | 0.861 | 0.57 | 6.023E−23 |
| ATP5J | 9.78E−39 | −0.375 | 0.675 | 0.386 | 9.532E−35 | CCDC109B | 6.20E−27 | 0.638 | 0.552 | 0.211 | 6.041E−23 |
| EIF3J | 9.86E−39 | −0.274 | 0.623 | 0.276 | 9.605E−35 | ACBD6 | 6.22E−27 | 0.795 | 0.578 | 0.202 | 6.064E−23 |
| NSA2 | 1.09E−38 | −0.357 | 0.502 | 0.175 | 1.060E−34 | CCDC137 | 8.99E−27 | 0.471 | 0.463 | 0.127 | 8.757E−23 |
| PHB | 1.20E−38 | −0.581 | 0.652 | 0.36 | 1.170E−34 | BUD31 | 9.99E−27 | 0.712 | 0.455 | 0.118 | 9.734E−23 |
| PSMB3 | 1.35E−38 | −0.823 | 0.573 | 0.346 | 1.312E−34 | XPO1 | 1.20E−26 | 0.512 | 0.578 | 0.241 | 1.172E−22 |
| CDC42EP1 | 1.55E−38 | −1.225 | 0.386 | 0.259 | 1.510E−34 | TARDBP | 1.38E−26 | 0.835 | 0.531 | 0.193 | 1.348E−22 |
| RPL27A | 1.69E−38 | −0.600 | 0.939 | 0.754 | 1.650E−34 | MAN1A2 | 1.41E−26 | 0.338 | 0.434 | 0.11 | 1.376E−22 |
| C19orf10 | 1.91E−38 | −0.791 | 0.57 | 0.311 | 1.863E−34 | XIST | 1.50E−26 | 0.965 | 0.52 | 0.167 | 1.466E−22 |
| CACYBP | 2.25E−38 | −0.426 | 0.915 | 0.671 | 2.191E−34 | CLU | 2.07E−26 | 0.407 | 0.511 | 0.193 | 2.021E−22 |
| CHMP4B | 2.40E−38 | −0.420 | 0.49 | 0.154 | 2.334E−34 | DPM1 | 2.59E−26 | 0.605 | 0.505 | 0.171 | 2.524E−22 |
| SNHG16 | 2.55E−38 | −0.634 | 0.466 | 0.189 | 2.481E−34 | MSMO1 | 2.60E−26 | 0.361 | 0.619 | 0.272 | 2.536E−22 |
| TXN | 2.61E−38 | −0.464 | 0.905 | 0.654 | 2.542E−34 | HIBADH | 2.77E−26 | 0.268 | 0.433 | 0.105 | 2.698E−22 |
| ATP5D | 3.83E−38 | −1.184 | 0.236 | 0.105 | 3.736E−34 | PLK2 | 2.83E−26 | 1.112 | 0.475 | 0.123 | 2.757E−22 |
| S100A16 | 3.94E−38 | −0.911 | 0.511 | 0.294 | 3.842E−34 | TMEM106C | 3.05E−26 | 0.599 | 0.564 | 0.224 | 2.974E−22 |
| COX7A2 | 4.80E−38 | −0.413 | 0.891 | 0.711 | 4.678E−34 | CCNC | 3.11E−26 | 0.363 | 0.492 | 0.175 | 3.035E−22 |
| HLA-E | 4.86E−38 | −0.967 | 0.573 | 0.412 | 4.738E−34 | NCOA7 | 3.22E−26 | 0.306 | 0.572 | 0.237 | 3.138E−22 |
| DCXR | 5.48E−38 | −1.054 | 0.384 | 0.18 | 5.343E−34 | ATXN10 | 3.86E−26 | 0.331 | 0.499 | 0.167 | 3.759E−22 |
| C16orf13 | 5.87E−38 | −0.465 | 0.49 | 0.211 | 5.724E−34 | HNRNPA2B1 | 4.07E−26 | 0.409 | 0.965 | 0.763 | 3.962E−22 |
| DYNLRB1 | 6.09E−38 | −0.280 | 0.511 | 0.154 | 5.931E−34 | RNF168 | 4.15E−26 | 0.312 | 0.434 | 0.123 | 4.046E−22 |
| CD52 | 9.44E−38 | −3.096 | 0.029 | 0.268 | 9.201E−34 | TOMM70A | 4.36E−26 | 0.391 | 0.484 | 0.171 | 4.250E−22 |
| NONO | 1.10E−37 | −0.726 | 0.48 | 0.189 | 1.069E−33 | NIPA2 | 4.85E−26 | 0.845 | 0.449 | 0.127 | 4.729E−22 |
| PLP2 | 1.21E−37 | −0.669 | 0.563 | 0.32 | 1.181E−33 | EIF2D | 4.99E−26 | 0.752 | 0.392 | 0.079 | 4.859E−22 |
| PSMB2 | 1.40E−37 | −0.260 | 0.641 | 0.307 | 1.361E−33 | G3BP2 | 5.59E−26 | 0.478 | 0.548 | 0.197 | 5.447E−22 |
| MT-TL1 | 1.41E−37 | −0.399 | 0.315 | 0.057 | 1.379E−33 | UBXN4 | 5.70E−26 | 0.274 | 0.531 | 0.211 | 5.559E−22 |
| HIST1H2BK | 1.46E−37 | −0.313 | 0.632 | 0.311 | 1.426E−33 | ZDHHC13 | 5.78E−26 | 0.636 | 0.472 | 0.132 | 5.634E−22 |
| S100A11 | 2.23E−37 | −0.567 | 0.958 | 0.82 | 2.172E−33 | LARP4B | 6.36E−26 | 0.253 | 0.475 | 0.154 | 6.199E−22 |
| LAMTOR2 | 2.75E−37 | −0.487 | 0.467 | 0.197 | 2.676E−33 | MTPN | 6.67E−26 | 0.289 | 0.443 | 0.14 | 6.503E−22 |
| WDR34 | 2.77E−37 | −0.821 | 0.46 | 0.237 | 2.704E−33 | CDCA7L | 7.07E−26 | 0.690 | 0.442 | 0.096 | 6.894E−22 |
| HSPD1 | 3.63E−37 | −0.291 | 0.915 | 0.658 | 3.534E−33 | MAP4 | 7.14E−26 | 0.427 | 0.504 | 0.175 | 6.962E−22 |
| SLC9A3R1 | 3.84E−37 | −0.739 | 0.49 | 0.25 | 3.742E−33 | VCP | 7.20E−26 | 0.401 | 0.483 | 0.171 | 7.020E−22 |
| RPL31 | 4.38E−37 | −0.669 | 0.929 | 0.772 | 4.268E−33 | LRPPRC | 7.32E−26 | 0.356 | 0.616 | 0.268 | 7.133E−22 |
| RPF2 | 4.62E−37 | −0.451 | 0.56 | 0.241 | 4.507E−33 | MRPS6 | 8.03E−26 | 0.430 | 0.425 | 0.101 | 7.827E−22 |
| COX5B | 5.05E−37 | −0.364 | 0.852 | 0.579 | 4.925E−33 | MKI67IP | 8.03E−26 | 0.483 | 0.449 | 0.11 | 7.830E−22 |
| MDH2 | 5.77E−37 | −0.619 | 0.492 | 0.254 | 5.619E−33 | FAM49B | 8.55E−26 | 0.763 | 0.431 | 0.114 | 8.333E−22 |
| NMT1 | 9.15E−37 | −0.798 | 0.408 | 0.136 | 8.921E−33 | CHMP5 | 1.03E−25 | 0.537 | 0.415 | 0.101 | 1.005E−21 |
| TCEB1 | 1.12E−36 | −0.616 | 0.502 | 0.215 | 1.088E−32 | NUDCD1 | 1.04E−25 | 0.481 | 0.533 | 0.202 | 1.010E−21 |
| KPNB1 | 1.38E−36 | −0.499 | 0.546 | 0.246 | 1.341E−32 | SETD3 | 1.21E−25 | 0.312 | 0.421 | 0.11 | 1.180E−21 |
| GRN | 1.45E−36 | −0.780 | 0.454 | 0.268 | 1.409E−32 | TSPAN6 | 1.31E−25 | 0.570 | 0.467 | 0.132 | 1.272E−21 |
| NDUFS2 | 1.61E−36 | −0.580 | 0.38 | 0.083 | 1.569E−32 | PNRC1 | 1.37E−25 | 0.293 | 0.41 | 0.088 | 1.335E−21 |
| DNAJB11 | 1.92E−36 | −0.725 | 0.446 | 0.224 | 1.870E−32 | 15-Sep | 1.53E−25 | 0.379 | 0.434 | 0.149 | 1.490E−21 |
| NDUFB3 | 1.96E−36 | −0.374 | 0.507 | 0.167 | 1.911E−32 | LPP | 1.54E−25 | 0.284 | 0.464 | 0.18 | 1.502E−21 |
| PPP2R1A | 3.03E−36 | −1.344 | 0.336 | 0.189 | 2.950E−32 | ADSS | 1.78E−25 | 0.364 | 0.536 | 0.202 | 1.737E−21 |
| EIF4E2 | 3.18E−36 | −0.269 | 0.526 | 0.215 | 3.098E−32 | MBP | 1.79E−25 | 0.253 | 0.469 | 0.145 | 1.745E−21 |
| CHMP1A | 3.34E−36 | −1.231 | 0.318 | 0.167 | 3.254E−32 | UCHL5 | 2.03E−25 | 0.484 | 0.672 | 0.338 | 1.980E−21 |
| BANF1 | 4.42E−36 | −0.862 | 0.359 | 0.123 | 4.310E−32 | TXNL1 | 2.04E−25 | 0.707 | 0.555 | 0.202 | 1.987E−21 |
| MAZ | 4.49E−36 | −1.615 | 0.257 | 0.158 | 4.377E−32 | HSP90B1 | 2.13E−25 | 0.264 | 0.933 | 0.68 | 2.080E−21 |
| MT-TV | 5.21E−36 | −0.565 | 0.253 | 0.053 | 5.077E−32 | SAR1A | 2.18E−25 | 0.627 | 0.484 | 0.149 | 2.120E−21 |
| NUDT5 | 5.63E−36 | −0.329 | 0.57 | 0.294 | 5.486E−32 | NUDT21 | 2.28E−25 | 0.440 | 0.457 | 0.136 | 2.221E−21 |
| RAD23A | 5.91E−36 | −0.520 | 0.581 | 0.276 | 5.762E−32 | ATP1B1 | 2.36E−25 | 0.295 | 0.511 | 0.206 | 2.296E−21 |
| CD9 | 1.01E−35 | −0.375 | 0.756 | 0.469 | 9.856E−32 | ARL3 | 2.39E−25 | 0.946 | 0.472 | 0.127 | 2.327E−21 |
| MYH9 | 1.03E−35 | −0.433 | 0.578 | 0.355 | 1.001E−31 | RFC1 | 2.52E−25 | 0.371 | 0.39 | 0.092 | 2.453E−21 |
| CDV3 | 1.06E−35 | −0.428 | 0.558 | 0.263 | 1.036E−31 | MYC | 2.55E−25 | 0.534 | 0.372 | 0.066 | 2.483E−21 |
| RER1 | 1.55E−35 | −0.628 | 0.467 | 0.224 | 1.508E−31 | PBX1 | 2.59E−25 | 0.590 | 0.396 | 0.101 | 2.522E−21 |
| EIF5A | 1.60E−35 | −0.719 | 0.377 | 0.149 | 1.556E−31 | PTTG1IP | 2.99E−25 | 0.418 | 0.876 | 0.588 | 2.912E−21 |
| NME4 | 1.63E−35 | −0.854 | 0.41 | 0.167 | 1.591E−31 | ICT1 | 3.15E−25 | 0.462 | 0.448 | 0.127 | 3.068E−21 |
| MT-CYB | 2.25E−35 | −0.472 | 0.974 | 0.816 | 2.194E−31 | TMED4 | 3.20E−25 | 0.632 | 0.508 | 0.171 | 3.123E−21 |
| IFI27L2 | 2.36E−35 | −0.984 | 0.274 | 0.083 | 2.301E−31 | SLC38A1 | 3.45E−25 | 0.394 | 0.829 | 0.531 | 3.366E−21 |
| TRMT112 | 3.08E−35 | −0.611 | 0.478 | 0.202 | 2.999E−31 | LASP1 | 3.82E−25 | 0.483 | 0.407 | 0.105 | 3.723E−21 |
| STK24 | 3.09E−35 | −0.360 | 0.523 | 0.25 | 3.015E−31 | TMEM126B | 4.07E−25 | 0.282 | 0.457 | 0.136 | 3.968E−21 |
| CID | 4.06E−35 | −0.718 | 0.266 | 0.061 | 3.952E−31 | ARFGAP3 | 4.22E−25 | 0.766 | 0.416 | 0.092 | 4.117E−21 |
| BTF3L4 | 4.14E−35 | −0.464 | 0.398 | 0.096 | 4.035E−31 | TSG101 | 4.45E−25 | 0.514 | 0.461 | 0.149 | 4.340E−21 |
| RAP1B | 4.35E−35 | −0.738 | 0.349 | 0.105 | 4.244E−31 | TIMMDC1 | 5.37E−25 | 1.103 | 0.43 | 0.096 | 5.231E−21 |
| RN7SKP36 | 4.46E−35 | −1.761 | 0.051 | 0.101 | 4.350E−31 | FGD5-AS1 | 5.49E−25 | 0.581 | 0.383 | 0.083 | 5.346E−21 |
| RP1- | 5.01E−35 | −1.345 | 0.103 | 0.053 | 4.885E−31 | HMG20B | 5.59E−25 | 0.959 | 0.508 | 0.162 | 5.452E−21 |
| 241P17.4 | | | | | | | | | | | |
| SF3B5 | 5.15E−35 | −0.488 | 0.434 | 0.149 | 5.021E−31 | C2orf15 | 6.26E−25 | 0.457 | 0.421 | 0.123 | 6.101E−21 |
| AES | 5.25E−35 | −0.435 | 0.455 | 0.154 | 5.118E−31 | SPATS2L | 7.96E−25 | 0.298 | 0.513 | 0.189 | 7.759E−21 |
| FKBP4 | 5.34E−35 | −0.256 | 0.477 | 0.167 | 5.206E−31 | RFK | 8.51E−25 | 0.815 | 0.399 | 0.083 | 8.289E−21 |
| TMED10 | 5.94E−35 | −0.316 | 0.643 | 0.338 | 5.793E−31 | GTF3A | 9.25E−25 | 0.722 | 0.814 | 0.465 | 9.011E−21 |
| AIF1L | 9.91E−35 | −0.432 | 0.592 | 0.294 | 9.656E−31 | CYB5R3 | 9.29E−25 | 0.334 | 0.36 | 0.127 | 9.054E−21 |
| EBP | 1.02E−34 | −0.274 | 0.499 | 0.184 | 9.928E−31 | PHF11 | 9.91E−25 | 0.343 | 0.466 | 0.154 | 9.660E−21 |
| MRPS7 | 2.85E−34 | −0.483 | 0.454 | 0.189 | 2.774E−30 | EIF2S1 | 1.04E−24 | 0.649 | 0.461 | 0.114 | 1.017E−20 |
| ISG15 | 2.88E−34 | −0.334 | 0.728 | 0.548 | 2.802E−30 | TTC17 | 1.54E−24 | 0.413 | 0.47 | 0.189 | 1.503E−20 |
| FAU | 3.46E−34 | −0.299 | 0.902 | 0.654 | 3.374E−30 | TMX4 | 1.75E−24 | 0.837 | 0.437 | 0.11 | 1.708E−20 |
| TMEM9 | 3.69E−34 | −0.371 | 0.502 | 0.228 | 3.593E−30 | ENOPH1 | 1.91E−24 | 0.765 | 0.389 | 0.088 | 1.861E−20 |
| CTSD | 3.95E−34 | −0.810 | 0.346 | 0.11 | 3.846E−30 | NCOA2 | 2.09E−24 | 0.513 | 0.413 | 0.114 | 2.034E−20 |
| MVD | 5.09E−34 | −0.786 | 0.368 | 0.132 | 4.962E−30 | CLINT1 | 2.11E−24 | 0.938 | 0.464 | 0.123 | 2.053E−20 |
| POLR2J | 6.22E−34 | −0.909 | 0.387 | 0.193 | 6.063E−30 | PCID2 | 2.28E−24 | 1.151 | 0.53 | 0.158 | 2.223E−20 |
| GPI | 6.58E−34 | −0.356 | 0.487 | 0.211 | 6.413E−30 | CDC42BPA | 2.36E−24 | 0.457 | 0.44 | 0.145 | 2.296E−20 |
| SELK | 6.80E−34 | −0.638 | 0.466 | 0.215 | 6.623E−30 | TMEM87A | 2.83E−24 | 0.313 | 0.411 | 0.118 | 2.756E−20 |
| PRKAR1A | 7.56E−34 | −0.501 | 0.516 | 0.246 | 7.367E−30 | CARS2 | 2.87E−24 | 1.118 | 0.38 | 0.066 | 2.793E−20 |
| LSM4 | 7.61E−34 | −0.498 | 0.437 | 0.162 | 7.412E−30 | FOS | 3.02E−24 | 1.591 | 0.29 | 0.013 | 2.945E−20 |
| TMEM205 | 8.57E−34 | −0.926 | 0.401 | 0.193 | 8.349E−30 | EIF3B | 3.06E−24 | 0.407 | 0.389 | 0.088 | 2.981E−20 |
| 9-Sep | 8.68E−34 | −0.713 | 0.434 | 0.197 | 8.460E−30 | RAB7A | 3.36E−24 | 0.263 | 0.475 | 0.18 | 3.277E−20 |
| RPSAP58 | 9.07E−34 | −1.653 | 0.129 | 0.088 | 8.839E−30 | TM9SF2 | 3.46E−24 | 0.533 | 0.932 | 0.693 | 3.370E−20 |
| UBE2D2 | 1.62E−33 | −0.402 | 0.484 | 0.167 | 1.577E−29 | OSTF1 | 3.69E−24 | 0.382 | 0.38 | 0.101 | 3.597E−20 |
| PRKX | 1.68E−33 | −0.323 | 0.517 | 0.206 | 1.640E−29 | SHROOM3 | 4.24E−24 | 1.620 | 0.333 | 0.035 | 4.134E−20 |
| EIF4H | 1.73E−33 | −0.834 | 0.369 | 0.162 | 1.686E−29 | TMEM106B | 4.28E−24 | 0.681 | 0.466 | 0.136 | 4.174E−20 |
| P4HB | 1.74E−33 | −0.425 | 0.874 | 0.645 | 1.696E−29 | TPD52 | 4.54E−24 | 0.337 | 0.402 | 0.118 | 4.427E−20 |
| LSM7 | 1.74E−33 | −0.351 | 0.593 | 0.325 | 1.699E−29 | LARP7 | 4.69E−24 | 0.394 | 0.427 | 0.132 | 4.570E−20 |
| HIGD1A | 1.83E−33 | −1.036 | 0.334 | 0.202 | 1.786E−29 | RPS21 | 4.89E−24 | 0.677 | 0.986 | 0.833 | 4.762E−20 |
| PADI2 | 2.42E−33 | −0.410 | 0.463 | 0.237 | 2.358E−29 | TMOD3 | 5.10E−24 | 0.283 | 0.483 | 0.189 | 4.971E−20 |
| DDAH2 | 2.50E−33 | −0.578 | 0.43 | 0.167 | 2.441E−29 | ETF1 | 5.56E−24 | 0.431 | 0.511 | 0.184 | 5.422E−20 |
| CLIC1 | 2.68E−33 | −0.746 | 0.374 | 0.167 | 2.607E−29 | ACIN1 | 6.70E−24 | 0.442 | 0.411 | 0.114 | 6.530E−20 |
| PFDN4 | 2.78E−33 | −1.208 | 0.399 | 0.259 | 2.705E−29 | NAA20 | 6.76E−24 | 0.401 | 0.454 | 0.175 | 6.584E−20 |
| PFDN1 | 2.80E−33 | −0.408 | 0.474 | 0.167 | 2.727E−29 | IQGAP1 | 6.97E−24 | 0.299 | 0.923 | 0.658 | 6.792E−20 |
| EIF4G1 | 2.87E−33 | −0.295 | 0.536 | 0.246 | 2.801E−29 | FMRI | 7.00E−24 | 0.431 | 0.44 | 0.136 | 6.826E−20 |
| NDUFS6 | 3.13E−33 | −0.341 | 0.938 | 0.746 | 3.050E−29 | GIGYF2 | 7.79E−24 | 0.527 | 0.384 | 0.096 | 7.596E−20 |
| RPL36A | 3.25E−33 | −0.702 | 0.271 | 0.07 | 3.168E−29 | TMPO | 9.11E−24 | 0.354 | 0.511 | 0.219 | 8.877E−20 |
| RPN2 | 3.89E−33 | −0.338 | 0.536 | 0.241 | 3.787E−29 | PTPRK | 1.06E−23 | 0.595 | 0.455 | 0.132 | 1.030E−19 |
| PPP1R2 | 4.93E−33 | −0.441 | 0.449 | 0.175 | 4.800E−29 | SMIM14 | 1.08E−23 | 0.640 | 0.34 | 0.057 | 1.053E−19 |
| RPL8 | 8.00E−33 | −0.448 | 0.921 | 0.772 | 7.798E−29 | YIPF4 | 1.18E−23 | 0.447 | 0.386 | 0.114 | 1.146E−19 |
| C1orf198 | 8.04E−33 | −0.473 | 0.517 | 0.232 | 7.832E−29 | CDC16 | 1.21E−23 | 0.411 | 0.496 | 0.175 | 1.177E−19 |
| METTL12 | 8.64E−33 | −0.834 | 0.225 | 0.083 | 8.421E−29 | FAM204A | 1.33E−23 | 0.262 | 0.411 | 0.127 | 1.298E−19 |
| LIMCH1 | 9.16E−33 | −0.295 | 0.643 | 0.32 | 8.929E−29 | SLC50A1 | 1.35E−23 | 0.364 | 0.436 | 0.14 | 1.318E−19 |
| DUSP18 | 1.08E−32 | −1.249 | 0.153 | 0.066 | 1.049E−28 | SYPL1 | 1.40E−23 | 0.812 | 0.425 | 0.105 | 1.368E−19 |
| NFE2L1 | 1.19E−32 | −0.358 | 0.534 | 0.259 | 1.158E−28 | CISDI | 1.48E−23 | 0.419 | 0.392 | 0.096 | 1.443E−19 |
| FAM192A | 1.20E−32 | −0.314 | 0.431 | 0.132 | 1.166E−28 | SMYD2 | 1.66E−23 | 0.413 | 0.393 | 0.092 | 1.620E−19 |
| ARHGDIB | 1.60E−32 | −0.636 | 0.536 | 0.329 | 1.558E−28 | DNAJC10 | 1.77E−23 | 0.485 | 0.463 | 0.162 | 1.721E−19 |
| SNRPF | 1.74E−32 | −0.338 | 0.434 | 0.154 | 1.699E−28 | TFG | 1.81E−23 | 0.570 | 0.495 | 0.167 | 1.767E−19 |
| NEDD8 | 1.90E−32 | −0.835 | 0.411 | 0.268 | 1.850E−28 | AZGP1 | 2.00E−23 | 0.811 | 0.953 | 0.776 | 1.949E−19 |
| IDH2 | 3.46E−32 | −0.255 | 0.843 | 0.605 | 3.374E−28 | MBTPS1 | 2.02E−23 | 0.668 | 0.427 | 0.118 | 1.967E−19 |
| IL32 | 3.55E−32 | −0.994 | 0.352 | 0.171 | 3.461E−28 | METTL9 | 2.20E−23 | 0.727 | 0.372 | 0.066 | 2.139E−19 |
| GLUL | 6.25E−32 | −0.509 | 0.48 | 0.259 | 6.090E−28 | CDKAL1 | 2.36E−23 | 0.260 | 0.495 | 0.202 | 2.296E−19 |
| WBP2 | 7.71E−32 | −0.803 | 0.354 | 0.158 | 7.515E−28 | PDHX | 2.39E−23 | 0.993 | 0.455 | 0.14 | 2.333E−19 |
| EIF5 | 8.33E−32 | −0.286 | 0.69 | 0.434 | 8.117E−28 | CD47 | 2.42E−23 | 0.261 | 0.939 | 0.715 | 2.359E−19 |
| SELT | 9.11E−32 | −0.832 | 0.377 | 0.197 | 8.876E−28 | PPCS | 2.52E−23 | 0.790 | 0.375 | 0.083 | 2.453E−19 |
| HSBP1L1 | 9.53E−32 | −0.635 | 0.401 | 0.14 | 9.291E−28 | HIST1H1C | 2.68E−23 | 0.345 | 0.72 | 0.404 | 2.610E−19 |
| PSMC6 | 9.58E−32 | −0.459 | 0.356 | 0.105 | 9.331E−28 | GGPS1 | 2.81E−23 | 0.400 | 0.496 | 0.18 | 2.743E−19 |
| AUP1 | 9.99E−32 | −0.854 | 0.349 | 0.149 | 9.736E−28 | TMEM30A | 2.84E−23 | 0.418 | 0.548 | 0.237 | 2.768E−19 |
| NBEAL1 | 1.06E−31 | −1.435 | 0.253 | 0.18 | 1.034E−27 | NSMCE2 | 3.30E−23 | 1.034 | 0.413 | 0.088 | 3.220E−19 |
| PHGDH | 1.14E−31 | −0.571 | 0.452 | 0.193 | 1.115E−27 | MPC1 | 3.42E−23 | 0.511 | 0.493 | 0.18 | 3.337E−19 |
| PTP4A2 | 1.15E−31 | −0.507 | 0.486 | 0.241 | 1.120E−27 | TIPRL | 3.92E−23 | 0.643 | 0.569 | 0.224 | 3.817E−19 |
| PPP1CA | 1.37E−31 | −0.336 | 0.467 | 0.18 | 1.338E−27 | ARPC5 | 4.39E−23 | 0.653 | 0.989 | 0.846 | 4.277E−19 |
| LAGE3 | 1.42E−31 | −0.342 | 0.454 | 0.149 | 1.381E−27 | EIF2B3 | 4.43E−23 | 0.455 | 0.408 | 0.127 | 4.318E−19 |
| SUMO3 | 1.57E−31 | −0.428 | 0.483 | 0.197 | 1.531E−27 | PTPN1 | 4.69E−23 | 0.686 | 0.396 | 0.092 | 4.570E−19 |
| FASN | 1.69E−31 | −0.563 | 0.395 | 0.211 | 1.649E−27 | UAP1 | 4.71E−23 | 0.397 | 0.371 | 0.114 | 4.592E−19 |
| QSOX1 | 2.73E−31 | −0.489 | 0.496 | 0.228 | 2.663E−27 | ZMAT2 | 4.83E−23 | 0.309 | 0.44 | 0.158 | 4.710E−19 |
| PCBP1 | 3.49E−31 | −0.434 | 0.454 | 0.158 | 3.401E−27 | TAF2 | 5.39E−23 | 0.269 | 0.425 | 0.145 | 5.253E−19 |
| PEA15 | 5.71E−31 | −0.348 | 0.513 | 0.232 | 5.561E−27 | UGT8 | 5.68E−23 | 0.714 | 0.427 | 0.123 | 5.538E−19 |
| STIP1 | 5.98E−31 | −0.276 | 0.585 | 0.263 | 5.825E−27 | ALDH1A3 | 5.82E−23 | 0.276 | 0.424 | 0.149 | 5.673E−19 |
| ASS1 | 9.33E−31 | −0.408 | 0.431 | 0.193 | 9.090E−27 | PLRG1 | 6.46E−23 | 0.407 | 0.399 | 0.11 | 6.294E−19 |
| MZT2B | 9.70E−31 | −0.723 | 0.466 | 0.254 | 9.451E−27 | PDS5A | 6.64E−23 | 0.314 | 0.425 | 0.132 | 6.472E−19 |
| NDUFS8 | 1.54E−30 | −0.754 | 0.321 | 0.132 | 1.498E−26 | SPEN | 6.96E−23 | 0.492 | 0.411 | 0.123 | 6.779E−19 |
| LINC00998 | 1.82E−30 | −0.467 | 0.436 | 0.175 | 1.777E−26 | ERLEC1 | 8.56E−23 | 0.604 | 0.455 | 0.136 | 8.341E−19 |
| DUSP23 | 1.96E−30 | −0.487 | 0.437 | 0.211 | 1.910E−26 | PMM2 | 9.69E−23 | 0.450 | 0.372 | 0.083 | 9.440E−19 |
| PSMB8 | 2.08E−30 | −0.272 | 0.525 | 0.254 | 2.031E−26 | MTHFD2 | 9.93E−23 | 0.415 | 0.449 | 0.149 | 9.678E−19 |
| PRDX3 | 2.39E−30 | −0.377 | 0.457 | 0.211 | 2.331E−26 | REEP3 | 1.29E−22 | 0.690 | 0.398 | 0.101 | 1.260E−18 |
| ATP6AP1 | 2.49E−30 | −0.511 | 0.418 | 0.237 | 2.423E−26 | ZNF638 | 1.53E−22 | 0.258 | 0.502 | 0.224 | 1.493E−18 |
| PPP4C | 2.87E−30 | −0.399 | 0.383 | 0.123 | 2.794E−26 | ZMYM4 | 1.57E−22 | 0.331 | 0.392 | 0.114 | 1.530E−18 |
| UXT | 2.93E−30 | −0.334 | 0.418 | 0.149 | 2.858E−26 | FAM208B | 1.61E−22 | 0.317 | 0.457 | 0.18 | 1.567E−18 |
| POLD2 | 3.27E−30 | −0.934 | 0.359 | 0.232 | 3.191E−26 | MRTO4 | 1.61E−22 | 0.365 | 0.366 | 0.088 | 1.573E−18 |
| HNRNPM | 3.28E−30 | −0.320 | 0.685 | 0.386 | 3.194E−26 | APPL1 | 1.78E−22 | 0.396 | 0.419 | 0.154 | 1.734E−18 |
| PSMB4 | 3.57E−30 | −0.279 | 0.539 | 0.254 | 3.478E−26 | PSMG2 | 1.81E−22 | 0.667 | 0.404 | 0.114 | 1.768E−18 |
| DRAP1 | 3.91E−30 | −0.366 | 0.392 | 0.123 | 3.806E−26 | PLGRKT | 1.90E−22 | 0.523 | 0.424 | 0.127 | 1.856E−18 |
| RAB11A | 4.00E−30 | −0.555 | 0.461 | 0.215 | 3.900E−26 | FBXW11 | 1.96E−22 | 0.547 | 0.372 | 0.088 | 1.911E−18 |
| GABARAPL2 | 4.02E−30 | −0.264 | 0.457 | 0.158 | 3.913E−26 | SDF4 | 2.01E−22 | 0.422 | 0.405 | 0.118 | 1.954E−18 |
| RPL35 | 4.59E−30 | −0.717 | 0.988 | 0.947 | 4.473E−26 | MARK2 | 2.23E−22 | 0.338 | 0.343 | 0.07 | 2.174E−18 |
| AP1S2 | 5.32E−30 | −0.611 | 0.356 | 0.158 | 5.187E−26 | LMO4 | 2.38E−22 | 0.502 | 0.363 | 0.11 | 2.316E−18 |
| SBDS | 5.70E−30 | −0.461 | 0.511 | 0.237 | 5.550E−26 | TRAP1 | 2.78E−22 | 0.297 | 0.352 | 0.092 | 2.710E−18 |
| SLC43A3 | 5.80E−30 | −0.711 | 0.351 | 0.127 | 5.648E−26 | SMC3 | 2.80E−22 | 0.280 | 0.448 | 0.162 | 2.730E−18 |
| TYMP | 5.80E−30 | −0.294 | 0.405 | 0.136 | 5.654E−26 | AC007390.5 | 2.87E−22 | 0.671 | 0.384 | 0.096 | 2.797E−18 |
| PRMT1 | 6.12E−30 | −0.998 | 0.263 | 0.101 | 5.963E−26 | HNRNPUL1 | 3.01E−22 | 0.401 | 0.404 | 0.145 | 2.937E−18 |
| UQCRB | 7.34E−30 | −0.500 | 0.424 | 0.154 | 7.156E−26 | ACLY | 3.09E−22 | 0.628 | 0.352 | 0.07 | 3.007E−18 |
| ADRM1 | 8.44E−30 | −0.589 | 0.33 | 0.105 | 8.225E−26 | CEBPG | 3.58E−22 | 0.562 | 0.434 | 0.132 | 3.487E−18 |
| CST3 | 8.65E−30 | −1.269 | 0.307 | 0.215 | 8.429E−26 | STX12 | 3.94E−22 | 0.484 | 0.386 | 0.101 | 3.840E−18 |
| PKM | 1.09E−29 | −0.476 | 0.508 | 0.268 | 1.060E−25 | SCOC | 4.02E−22 | 0.482 | 0.413 | 0.118 | 3.918E−18 |
| AP1B1 | 1.15E−29 | −0.682 | 0.34 | 0.14 | 1.124E−25 | DENND1B | 4.32E−22 | 0.704 | 0.411 | 0.127 | 4.206E−18 |
| ATP6V1F | 1.43E−29 | −0.452 | 0.306 | 0.07 | 1.395E−25 | ITGB8 | 4.42E−22 | 0.396 | 0.431 | 0.145 | 4.305E−18 |
| NAMPT | 1.48E−29 | −0.308 | 0.398 | 0.123 | 1.445E−25 | GOLGB1 | 4.68E−22 | 0.363 | 0.389 | 0.118 | 4.559E−18 |
| CRIP2 | 1.50E−29 | −0.332 | 0.374 | 0.14 | 1.464E−25 | SNHG1 | 4.89E−22 | 0.284 | 0.283 | 0.053 | 4.767E−18 |
| RAB5C | 1.90E−29 | −1.068 | 0.269 | 0.123 | 1.849E−25 | VAMP7 | 4.94E−22 | 0.384 | 0.405 | 0.123 | 4.817E−18 |
| PTBP1 | 1.93E−29 | −0.649 | 0.365 | 0.136 | 1.878E−25 | IARS | 5.29E−22 | 0.309 | 0.375 | 0.114 | 5.153E−18 |
| TRAPPC3 | 1.98E−29 | −0.292 | 0.489 | 0.206 | 1.933E−25 | ATG5 | 5.35E−22 | 0.483 | 0.421 | 0.11 | 5.210E−18 |
| EIF3F | 2.05E−29 | −1.290 | 0.281 | 0.154 | 2.001E−25 | SETX | 5.56E−22 | 0.531 | 0.421 | 0.118 | 5.421E−18 |
| RPL24 | 2.13E−29 | −0.396 | 0.979 | 0.789 | 2.076E−25 | SNRNP200 | 5.57E−22 | 0.465 | 0.34 | 0.066 | 5.430E−18 |
| UTP11L | 2.19E−29 | −0.422 | 0.504 | 0.25 | 2.131E−25 | MINA | 5.73E−22 | 0.635 | 0.372 | 0.088 | 5.583E−18 |
| ZNF90 | 2.19E−29 | −1.296 | 0.168 | 0.092 | 2.136E−25 | RP11- | 5.95E−22 | 0.398 | 0.268 | 0.026 | 5.796E−18 |
| | | | | | | 85F14.5 | | | | | |
| EIF4E | 2.23E−29 | −0.957 | 0.293 | 0.14 | 2.175E−25 | MRPL3 | 6.85E−22 | 0.297 | 0.402 | 0.127 | 6.672E−18 |
| CAPNS1 | 2.69E−29 | −0.784 | 0.395 | 0.202 | 2.626E−25 | UBAC1 | 8.96E−22 | 1.048 | 0.34 | 0.057 | 8.732E−18 |
| EIF3K | 2.77E−29 | −0.320 | 0.51 | 0.241 | 2.699E−25 | PSMA7 | 9.10E−22 | 0.337 | 0.918 | 0.658 | 8.864E−18 |
| HNRNPH1 | 2.89E−29 | −0.408 | 0.489 | 0.206 | 2.821E−25 | XRN2 | 1.01E−21 | 0.375 | 0.463 | 0.175 | 9.835E−18 |
| CTSB | 3.04E−29 | −0.888 | 0.448 | 0.272 | 2.966E−25 | IFI44 | 1.04E−21 | 0.668 | 0.313 | 0.061 | 1.016E−17 |
| TMEM141 | 3.37E−29 | −0.541 | 0.377 | 0.136 | 3.288E−25 | GTF2H1 | 1.08E−21 | 0.539 | 0.377 | 0.105 | 1.050E−17 |
| MYEOV2 | 4.35E−29 | −0.287 | 0.811 | 0.526 | 4.236E−25 | TRIP12 | 1.08E−21 | 0.261 | 0.449 | 0.18 | 1.053E−17 |
| AKR1A1 | 4.58E−29 | −0.386 | 0.424 | 0.158 | 4.463E−25 | HPS3 | 1.22E−21 | 0.681 | 0.439 | 0.145 | 1.190E−17 |
| ACTN1 | 4.61E−29 | −0.301 | 0.467 | 0.206 | 4.492E−25 | ADK | 1.24E−21 | 0.954 | 0.431 | 0.123 | 1.208E−17 |
| LINC00493 | 4.88E−29 | −0.495 | 0.393 | 0.158 | 4.752E−25 | ATP6V1C1 | 1.42E−21 | 0.473 | 0.369 | 0.092 | 1.384E−17 |
| UQCRC1 | 4.90E−29 | −0.275 | 0.421 | 0.175 | 4.776E−25 | EIF3D | 1.53E−21 | 0.320 | 0.363 | 0.096 | 1.493E−17 |
| PSMD4 | 5.26E−29 | −0.376 | 0.526 | 0.268 | 5.127E−25 | PSMD12 | 1.75E−21 | 0.330 | 0.519 | 0.232 | 1.707E−17 |
| CFDP1 | 5.67E−29 | −0.459 | 0.357 | 0.096 | 5.521E−25 | PDIA6 | 1.75E−21 | 0.385 | 0.829 | 0.553 | 1.708E−17 |
| COA3 | 6.29E−29 | −0.523 | 0.352 | 0.154 | 6.129E−25 | MCTS1 | 1.76E−21 | 0.523 | 0.346 | 0.07 | 1.711E−17 |
| PABPC1 | 6.89E−29 | −0.728 | 0.995 | 0.987 | 6.711E−25 | MAN1A1 | 1.76E−21 | 0.614 | 0.366 | 0.101 | 1.713E−17 |
| HCFC1R1 | 7.97E−29 | −0.846 | 0.284 | 0.105 | 7.770E−25 | POLE3 | 1.96E−21 | 0.606 | 0.415 | 0.11 | 1.908E−17 |
| MT-ND4 | 8.63E−29 | −0.934 | 0.98 | 0.912 | 8.407E−25 | USP34 | 1.97E−21 | 0.653 | 0.407 | 0.132 | 1.922E−17 |
| TTC19 | 1.00E−28 | −0.654 | 0.3 | 0.136 | 9.781E−25 | ANAPC5 | 2.05E−21 | 0.746 | 0.484 | 0.171 | 1.999E−17 |
| HLA-DRA | 1.02E−28 | −1.463 | 0.225 | 0.259 | 9.941E−25 | CHAMP1 | 2.06E−21 | 0.327 | 0.398 | 0.127 | 2.007E−17 |
| SSNA1 | 1.02E−28 | −0.316 | 0.389 | 0.132 | 9.988E−25 | DENR | 2.31E−21 | 0.408 | 0.421 | 0.123 | 2.252E−17 |
| PPP2R4 | 1.05E−28 | −0.744 | 0.342 | 0.14 | 1.019E−24 | USP7 | 2.46E−21 | 0.396 | 0.372 | 0.118 | 2.399E−17 |
| NUDC | 1.12E−28 | −0.704 | 0.455 | 0.215 | 1.094E−24 | GTF2F2 | 2.61E−21 | 0.854 | 0.356 | 0.066 | 2.543E−17 |
| TOMM5 | 1.35E−28 | −1.338 | 0.132 | 0.066 | 1.314E−24 | GTF2I | 2.61E−21 | 0.631 | 0.351 | 0.083 | 2.548E−17 |
| ANP32E | 1.44E−28 | −0.365 | 0.493 | 0.241 | 1.407E−24 | UFD1L | 2.70E−21 | 0.447 | 0.384 | 0.105 | 2.636E−17 |
| LAPTM4B | 1.54E−28 | −0.402 | 0.454 | 0.197 | 1.505E−24 | PPP1CC | 3.05E−21 | 0.424 | 0.474 | 0.193 | 2.977E−17 |
| PSMB1 | 1.59E−28 | −0.447 | 0.489 | 0.241 | 1.553E−24 | RNASEH2B | 3.14E−21 | 0.705 | 0.334 | 0.061 | 3.062E−17 |
| FAM211A | 1.68E−28 | −0.932 | 0.234 | 0.07 | 1.639E−24 | TCERG1 | 3.14E−21 | 0.587 | 0.325 | 0.057 | 3.063E−17 |
| HLA-F | 2.09E−28 | −0.733 | 0.351 | 0.171 | 2.033E−24 | NOL11 | 3.23E−21 | 0.352 | 0.343 | 0.088 | 3.147E−17 |
| PAIP1 | 2.86E−28 | −0.666 | 0.268 | 0.083 | 2.792E−24 | ACAA2 | 3.35E−21 | 0.342 | 0.298 | 0.057 | 3.266E−17 |
| PSMC4 | 3.34E−28 | −0.450 | 0.375 | 0.158 | 3.251E−24 | ARL8A | 4.13E−21 | 0.661 | 0.393 | 0.123 | 4.029E−17 |
| DTYMK | 3.66E−28 | −0.746 | 0.39 | 0.197 | 3.564E−24 | SRD5A3 | 4.81E−21 | 0.298 | 0.366 | 0.092 | 4.692E−17 |
| ARPC1A | 3.76E−28 | −0.690 | 0.331 | 0.105 | 3.660E−24 | MICU2 | 5.13E−21 | 0.772 | 0.325 | 0.053 | 5.003E−17 |
| SUMO1 | 4.19E−28 | −0.319 | 0.393 | 0.127 | 4.084E−24 | GOLPH3 | 5.14E−21 | 0.615 | 0.356 | 0.079 | 5.010E−17 |
| ANAPC16 | 4.20E−28 | −0.674 | 0.395 | 0.184 | 4.097E−24 | LPL | 5.18E−21 | 0.284 | 0.365 | 0.092 | 5.051E−17 |
| TTYH1 | 4.56E−28 | −0.332 | 0.356 | 0.11 | 4.442E−24 | HPRT1 | 5.36E−21 | 0.540 | 0.398 | 0.105 | 5.221E−17 |
| MYADM | 4.68E−28 | −0.416 | 0.43 | 0.197 | 4.557E−24 | MAGED1 | 6.07E−21 | 0.530 | 0.371 | 0.092 | 5.919E−17 |
| RP11- | 5.23E−28 | −1.389 | 0.11 | 0.088 | 5.096E−24 | SCAND1 | 6.12E−21 | 0.308 | 0.366 | 0.132 | 5.959E−17 |
| 58E21.4 | | | | | | | | | | | |
| HLA-DRB1 | 6.83E−28 | −1.261 | 0.225 | 0.175 | 6.657E−24 | METAP1 | 6.22E−21 | 0.311 | 0.339 | 0.083 | 6.058E−17 |
| AK4 | 6.86E−28 | −0.537 | 0.372 | 0.154 | 6.689E−24 | TIFA | 6.84E−21 | 0.467 | 0.34 | 0.083 | 6.663E−17 |
| DCTPP1 | 6.93E−28 | −0.495 | 0.389 | 0.145 | 6.751E−24 | RIOK3 | 7.71E−21 | 0.519 | 0.469 | 0.171 | 7.510E−17 |
| MRPL24 | 7.27E−28 | −0.367 | 0.445 | 0.189 | 7.087E−24 | RARS2 | 8.33E−21 | 0.354 | 0.343 | 0.083 | 8.115E−17 |
| CD81 | 8.48E−28 | −1.436 | 0.224 | 0.132 | 8.262E−24 | FAIM | 8.58E−21 | 1.109 | 0.356 | 0.088 | 8.360E−17 |
| B4GALT1 | 9.44E−28 | −0.320 | 0.396 | 0.136 | 9.200E−24 | HMGCR | 9.66E−21 | 0.461 | 0.346 | 0.075 | 9.417E−17 |
| PRR13 | 1.04E−27 | −1.272 | 0.221 | 0.114 | 1.014E−23 | SCYL2 | 1.06E−20 | 0.503 | 0.352 | 0.083 | 1.032E−16 |
| NDUFA12 | 1.05E−27 | −0.478 | 0.436 | 0.193 | 1.026E−23 | SLC30A9 | 1.30E−20 | 0.721 | 0.419 | 0.154 | 1.267E−16 |
| DNAJC9 | 1.12E−27 | −0.315 | 0.289 | 0.061 | 1.096E−23 | USP14 | 1.34E−20 | 0.483 | 0.437 | 0.171 | 1.305E−16 |
| SRGN | 1.20E−27 | −2.291 | 0.047 | 0.175 | 1.167E−23 | VASP | 1.47E−20 | 0.601 | 0.284 | 0.039 | 1.432E−16 |
| DDX46 | 1.24E−27 | −0.662 | 0.446 | 0.228 | 1.204E−23 | MRPS5 | 1.53E−20 | 0.366 | 0.436 | 0.171 | 1.488E−16 |
| FIS1 | 1.24E−27 | −0.768 | 0.301 | 0.096 | 1.208E−23 | MIB1 | 1.59E−20 | 0.264 | 0.339 | 0.096 | 1.548E−16 |
| TUBB4B | 1.27E−27 | −0.255 | 0.433 | 0.184 | 1.236E−23 | TSC22D1 | 1.62E−20 | 0.645 | 0.36 | 0.088 | 1.581E−16 |
| MSN | 1.28E−27 | −0.393 | 0.452 | 0.167 | 1.251E−23 | C12orf23 | 1.66E−20 | 0.855 | 0.375 | 0.088 | 1.622E−16 |
| SUPT4H1 | 1.31E−27 | −0.589 | 0.408 | 0.197 | 1.281E−23 | MYEF2 | 1.75E−20 | 0.757 | 0.298 | 0.044 | 1.707E−16 |
| CAPN1 | 1.38E−27 | −0.560 | 0.306 | 0.105 | 1.341E−23 | GSTO1 | 1.96E−20 | 0.311 | 0.384 | 0.14 | 1.905E−16 |
| RPL34 | 1.38E−27 | −0.683 | 0.955 | 0.842 | 1.344E−23 | 10-Sep | 1.98E−20 | 0.746 | 0.331 | 0.066 | 1.928E−16 |
| NELFE | 1.52E−27 | −0.577 | 0.377 | 0.158 | 1.484E−23 | ERRFI1 | 2.00E−20 | 0.541 | 0.408 | 0.14 | 1.944E−16 |
| BLOC1S1 | 1.63E−27 | −0.816 | 0.3 | 0.11 | 1.587E−23 | ARL2BP | 2.12E−20 | 0.500 | 0.36 | 0.083 | 2.066E−16 |
| FKBP1A | 1.67E−27 | −0.921 | 0.278 | 0.127 | 1.626E−23 | STAMBP | 2.18E−20 | 0.855 | 0.366 | 0.083 | 2.121E−16 |
| TIMM17B | 1.79E−27 | −0.393 | 0.368 | 0.145 | 1.743E−23 | ZC3H14 | 2.25E−20 | 0.617 | 0.281 | 0.035 | 2.189E−16 |
| AP1S1 | 1.81E−27 | −0.603 | 0.275 | 0.079 | 1.763E−23 | PSMC2 | 2.32E−20 | 0.250 | 0.363 | 0.11 | 2.257E−16 |
| GNAI2 | 2.14E−27 | −0.407 | 0.354 | 0.132 | 2.088E−23 | PNPT1 | 2.38E−20 | 0.514 | 0.442 | 0.193 | 2.323E−16 |
| PNKD | 2.18E−27 | −0.609 | 0.366 | 0.167 | 2.121E−23 | ATP2C1 | 2.42E−20 | 0.707 | 0.362 | 0.083 | 2.354E−16 |
| CALM3 | 2.25E−27 | −0.257 | 0.487 | 0.268 | 2.197E−23 | TANK | 2.42E−20 | 0.620 | 0.398 | 0.132 | 2.360E−16 |
| FAM136A | 2.49E−27 | −0.340 | 0.398 | 0.149 | 2.422E−23 | NCK1 | 2.58E−20 | 0.579 | 0.345 | 0.075 | 2.515E−16 |
| AHCTF1 | 2.57E−27 | −0.715 | 0.31 | 0.11 | 2.502E−23 | HDDC2 | 2.76E−20 | 0.388 | 0.374 | 0.114 | 2.693E−16 |
| QPRT | 2.64E−27 | −0.608 | 0.384 | 0.18 | 2.572E−23 | STARD3NL | 2.77E−20 | 0.671 | 0.319 | 0.061 | 2.700E−16 |
| NDUFAF2 | 2.83E−27 | −0.615 | 0.318 | 0.096 | 2.758E−23 | COPS8 | 2.79E−20 | 0.282 | 0.328 | 0.079 | 2.717E−16 |
| GPS1 | 3.06E−27 | −0.705 | 0.357 | 0.167 | 2.982E−23 | SLC12A2 | 2.91E−20 | 0.466 | 0.398 | 0.118 | 2.834E−16 |
| APOA1BP | 3.32E−27 | −0.324 | 0.407 | 0.167 | 3.234E−23 | NANS | 3.20E−20 | 0.626 | 0.304 | 0.057 | 3.114E−16 |
| SERF2 | 3.55E−27 | −0.799 | 0.976 | 0.947 | 3.460E−23 | PIK3R3 | 3.24E−20 | 0.383 | 0.386 | 0.118 | 3.155E−16 |
| KATNBL1 | 3.57E−27 | −0.482 | 0.33 | 0.127 | 3.480E−23 | N4BP2L2 | 3.25E−20 | 0.387 | 0.44 | 0.175 | 3.170E−16 |
| GNB2 | 3.70E−27 | −0.419 | 0.295 | 0.07 | 3.604E−23 | COBL | 3.30E−20 | 0.529 | 0.383 | 0.132 | 3.219E−16 |
| POLR2E | 3.73E−27 | −0.578 | 0.336 | 0.123 | 3.639E−23 | KIAA0368 | 3.52E−20 | 0.279 | 0.411 | 0.145 | 3.432E−16 |
| CCDC58 | 4.14E−27 | −0.312 | 0.289 | 0.044 | 4.037E−23 | ZNF124 | 3.66E−20 | 0.529 | 0.313 | 0.066 | 3.569E−16 |
| NDUFA2 | 4.36E−27 | −0.435 | 0.472 | 0.228 | 4.248E−23 | CMTM6 | 3.78E−20 | 0.463 | 0.451 | 0.171 | 3.688E−16 |
| AMZ2 | 5.64E−27 | −0.290 | 0.374 | 0.123 | 5.494E−23 | CRIPT | 3.94E−20 | 0.325 | 0.306 | 0.07 | 3.835E−16 |
| SLC1A5 | 6.21E−27 | −0.658 | 0.316 | 0.14 | 6.054E−23 | SERPINE2 | 4.06E−20 | 0.588 | 0.339 | 0.079 | 3.956E−16 |
| CKS2 | 6.22E−27 | −0.535 | 0.486 | 0.373 | 6.060E−23 | PPP1R15B | 4.23E−20 | 0.334 | 0.34 | 0.096 | 4.126E−16 |
| CCDC124 | 6.72E−27 | −1.176 | 0.219 | 0.088 | 6.549E−23 | DNAJA2 | 4.27E−20 | 0.583 | 0.342 | 0.083 | 4.158E−16 |
| GPR56 | 7.86E−27 | −0.499 | 0.334 | 0.114 | 7.656E−23 | SF3A3 | 4.32E−20 | 0.698 | 0.301 | 0.044 | 4.207E−16 |
| SLC44A2 | 8.93E−27 | −0.726 | 0.415 | 0.246 | 8.701E−23 | EMC8 | 4.48E−20 | 0.479 | 0.374 | 0.096 | 4.368E−16 |
| MRPS10 | 9.60E−27 | −0.323 | 0.401 | 0.158 | 9.358E−23 | EMC2 | 4.56E−20 | 0.574 | 0.334 | 0.088 | 4.440E−16 |
| LAMTOR4 | 9.61E−27 | −0.376 | 0.331 | 0.101 | 9.366E−23 | REEP5 | 5.73E−20 | 0.719 | 0.398 | 0.114 | 5.585E−16 |
| YTHDF2 | 1.00E−26 | −0.839 | 0.318 | 0.18 | 9.781E−23 | CCDC6 | 6.08E−20 | 0.520 | 0.405 | 0.145 | 5.928E−16 |
| RANBP2 | 1.02E−26 | −0.594 | 0.316 | 0.101 | 9.936E−23 | FAM120AOS | 6.21E−20 | 0.715 | 0.369 | 0.092 | 6.050E−16 |
| AAMP | 1.04E−26 | −0.957 | 0.3 | 0.127 | 1.010E−22 | DIEXF | 6.55E−20 | 0.359 | 0.336 | 0.079 | 6.387E−16 |
| TMEM147 | 1.12E−26 | −0.465 | 0.393 | 0.154 | 1.089E−22 | NFKBIZ | 6.70E−20 | 0.441 | 0.256 | 0.044 | 6.533E−16 |
| RNPS1 | 1.18E−26 | −0.828 | 0.318 | 0.145 | 1.147E−22 | ARRDC3 | 6.75E−20 | 0.468 | 0.34 | 0.096 | 6.582E−16 |
| SYF2 | 1.28E−26 | −0.679 | 0.369 | 0.18 | 1.252E−22 | CNN3 | 7.13E−20 | 0.324 | 0.416 | 0.184 | 6.949E−16 |
| NBPF10 | 1.57E−26 | −1.476 | 0.142 | 0.079 | 1.531E−22 | ZNF511 | 7.90E−20 | 1.065 | 0.351 | 0.066 | 7.697E−16 |
| DCTN3 | 1.59E−26 | −0.335 | 0.371 | 0.127 | 1.552E−22 | RNF10 | 8.13E−20 | 0.412 | 0.306 | 0.057 | 7.927E−16 |
| C8orf59 | 1.75E−26 | −0.292 | 0.421 | 0.167 | 1.710E−22 | MAPRE1 | 8.44E−20 | 0.266 | 0.337 | 0.088 | 8.226E−16 |
| DNAJB1 | 2.20E−26 | −0.589 | 0.436 | 0.215 | 2.145E−22 | STK38 | 8.58E−20 | 0.664 | 0.321 | 0.066 | 8.360E−16 |
| UBE2L6 | 2.71E−26 | −0.522 | 0.334 | 0.136 | 2.637E−22 | UROS | 8.67E−20 | 0.497 | 0.377 | 0.101 | 8.452E−16 |
| S100A4 | 2.92E−26 | −1.894 | 0.11 | 0.189 | 2.845E−22 | CCNL1 | 8.79E−20 | 0.887 | 0.398 | 0.114 | 8.570E−16 |
| PTPN14 | 3.25E−26 | −0.280 | 0.401 | 0.158 | 3.168E−22 | GNL2 | 9.58E−20 | 0.260 | 0.369 | 0.127 | 9.336E−16 |
| YIF1A | 3.48E−26 | −0.797 | 0.349 | 0.171 | 3.395E−22 | FAM98A | 1.03E−19 | 0.640 | 0.287 | 0.057 | 1.003E−15 |
| IDH3G | 3.69E−26 | −0.283 | 0.36 | 0.11 | 3.595E−22 | TMX1 | 1.05E−19 | 0.780 | 0.356 | 0.075 | 1.027E−15 |
| AP3S1 | 3.89E−26 | −0.261 | 0.352 | 0.11 | 3.786E−22 | HSPA4 | 1.09E−19 | 0.562 | 0.401 | 0.14 | 1.062E−15 |
| LRRFIP1 | 4.05E−26 | −0.332 | 0.564 | 0.325 | 3.949E−22 | DARS | 1.14E−19 | 0.330 | 0.387 | 0.136 | 1.115E−15 |
| OIP5-AS1 | 4.35E−26 | −0.449 | 0.411 | 0.18 | 4.237E−22 | LZIC | 1.23E−19 | 0.854 | 0.29 | 0.048 | 1.197E−15 |
| MRPL55 | 4.85E−26 | −0.528 | 0.407 | 0.18 | 4.725E−22 | MBNL1 | 1.31E−19 | 0.448 | 0.49 | 0.206 | 1.274E−15 |
| MRPS12 | 4.89E−26 | −0.474 | 0.322 | 0.123 | 4.770E−22 | NLRP2 | 1.40E−19 | 0.373 | 0.349 | 0.092 | 1.367E−15 |
| NSDHL | 4.92E−26 | −0.482 | 0.36 | 0.105 | 4.790E−22 | TXN2 | 1.56E−19 | 0.252 | 0.34 | 0.105 | 1.516E−15 |
| PMVK | 5.82E−26 | −0.514 | 0.309 | 0.092 | 5.672E−22 | SUCO | 1.60E−19 | 0.544 | 0.449 | 0.162 | 1.557E−15 |
| MT-ND6 | 6.17E−26 | −0.899 | 0.274 | 0.136 | 6.016E−22 | UFL1 | 2.01E−19 | 0.253 | 0.346 | 0.105 | 1.957E−15 |
| HADH | 6.34E−26 | −0.266 | 0.478 | 0.206 | 6.178E−22 | TPD52L1 | 2.27E−19 | 0.719 | 0.351 | 0.105 | 2.212E−15 |
| IK | 6.89E−26 | −0.682 | 0.33 | 0.123 | 6.712E−22 | TPT1-AS1 | 2.33E−19 | 0.419 | 0.277 | 0.048 | 2.272E−15 |
| NDUFS7 | 6.94E−26 | −0.617 | 0.359 | 0.158 | 6.767E−22 | CCDC59 | 2.54E−19 | 0.277 | 0.333 | 0.092 | 2.475E−15 |
| MYL6 | 7.78E−26 | −0.327 | 0.938 | 0.789 | 7.579E−22 | CPSF6 | 2.58E−19 | 0.791 | 0.303 | 0.053 | 2.519E−15 |
| ERI3 | 8.07E−26 | −1.230 | 0.201 | 0.083 | 7.863E−22 | KCNK1 | 2.90E−19 | 0.456 | 0.342 | 0.083 | 2.825E−15 |
| SEC31B | 8.46E−26 | −1.480 | 0.169 | 0.101 | 8.241E−22 | MB21D1 | 2.97E−19 | 0.462 | 0.328 | 0.079 | 2.891E−15 |
| MLF2 | 8.51E−26 | −0.556 | 0.41 | 0.18 | 8.297E−22 | EXOC3 | 2.97E−19 | 0.861 | 0.356 | 0.092 | 2.893E−15 |
| PSMD7 | 9.75E−26 | −0.369 | 0.489 | 0.259 | 9.502E−22 | SPG21 | 3.19E−19 | 0.528 | 0.363 | 0.105 | 3.112E−15 |
| RSRC1 | 1.07E−25 | −0.448 | 0.461 | 0.254 | 1.044E−21 | TPP2 | 3.52E−19 | 0.467 | 0.402 | 0.136 | 3.430E−15 |
| MPHOSPH8 | 1.17E−25 | −0.742 | 0.265 | 0.07 | 1.139E−21 | KIAA1598 | 4.07E−19 | 0.489 | 0.349 | 0.101 | 3.967E−15 |
| DHCR7 | 1.20E−25 | −0.406 | 0.318 | 0.114 | 1.174E−21 | EZH2 | 4.31E−19 | 0.613 | 0.316 | 0.066 | 4.205E−15 |
| HLA-DPA1 | 1.33E−25 | −1.901 | 0.159 | 0.162 | 1.294E−21 | NMD3 | 4.83E−19 | 0.266 | 0.34 | 0.083 | 4.705E−15 |
| EMP2 | 1.63E−25 | −0.433 | 0.369 | 0.14 | 1.592E−21 | MRPS22 | 5.05E−19 | 0.383 | 0.331 | 0.083 | 4.916E−15 |
| TMA16 | 1.68E−25 | −0.481 | 0.365 | 0.149 | 1.636E−21 | ADSL | 5.15E−19 | 0.554 | 0.25 | 0.039 | 5.021E−15 |
| CDK16 | 1.76E−25 | −0.411 | 0.354 | 0.11 | 1.714E−21 | RPS6 | 6.07E−19 | 0.529 | 0.998 | 0.952 | 5.915E−15 |
| ARF4 | 1.77E−25 | −0.382 | 0.422 | 0.171 | 1.726E−21 | DHX40 | 6.37E−19 | 0.641 | 0.269 | 0.044 | 6.208E−15 |
| BACE2 | 1.87E−25 | −0.441 | 0.372 | 0.132 | 1.824E−21 | DIP2A | 7.09E−19 | 0.386 | 0.325 | 0.088 | 6.911E−15 |
| WBP11 | 1.88E−25 | −0.592 | 0.349 | 0.132 | 1.830E−21 | TSR2 | 7.48E−19 | 0.566 | 0.395 | 0.14 | 7.292E−15 |
| C19orf24 | 2.30E−25 | −0.819 | 0.263 | 0.096 | 2.241E−21 | NAA15 | 7.63E−19 | 0.377 | 0.407 | 0.132 | 7.432E−15 |
| MIEN1 | 2.35E−25 | −0.556 | 0.368 | 0.136 | 2.285E−21 | MMAB | 7.70E−19 | 0.335 | 0.368 | 0.132 | 7.506E−15 |
| EXOSC4 | 2.52E−25 | −1.047 | 0.274 | 0.154 | 2.451E−21 | PRPF3 | 7.81E−19 | 0.786 | 0.366 | 0.088 | 7.616E−15 |
| CBR1 | 2.60E−25 | −0.364 | 0.387 | 0.145 | 2.533E−21 | SEPPI | 8.06E−19 | 0.417 | 0.349 | 0.101 | 7.852E−15 |
| EIF6 | 2.91E−25 | −0.797 | 0.292 | 0.132 | 2.832E−21 | TIMM10 | 8.49E−19 | 0.718 | 0.315 | 0.066 | 8.273E−15 |
| TBCB | 2.92E−25 | −0.656 | 0.34 | 0.145 | 2.843E−21 | MANF | 8.61E−19 | 0.710 | 0.292 | 0.057 | 8.394E−15 |
| PABPC4 | 2.93E−25 | −0.488 | 0.322 | 0.114 | 2.854E−21 | PPP2R3A | 8.69E−19 | 0.324 | 0.351 | 0.101 | 8.471E−15 |
| CTSC | 3.22E−25 | −0.592 | 0.374 | 0.171 | 3.134E−21 | WNK1 | 8.92E−19 | 0.405 | 0.331 | 0.083 | 8.688E−15 |
| SHISA5 | 3.63E−25 | −0.673 | 0.334 | 0.158 | 3.535E−21 | MAPK9 | 9.27E−19 | 0.306 | 0.289 | 0.061 | 9.038E−15 |
| CERS2 | 4.00E−25 | −0.383 | 0.419 | 0.158 | 3.902E−21 | NSL1 | 1.16E−18 | 0.740 | 0.415 | 0.127 | 1.131E−14 |
| SLC39A1 | 4.80E−25 | −0.256 | 0.418 | 0.197 | 4.673E−21 | GATAD1 | 1.18E−18 | 0.449 | 0.272 | 0.048 | 1.150E−14 |
| SMG1 | 4.82E−25 | −0.317 | 0.337 | 0.127 | 4.701E−21 | DDX27 | 1.32E−18 | 0.528 | 0.31 | 0.066 | 1.288E−14 |
| CAST | 4.84E−25 | −0.346 | 0.434 | 0.232 | 4.721E−21 | CELF1 | 1.33E−18 | 0.275 | 0.319 | 0.088 | 1.293E−14 |
| PDCD4 | 5.06E−25 | −0.731 | 0.401 | 0.206 | 4.933E−21 | TRA2A | 1.42E−18 | 0.345 | 0.336 | 0.101 | 1.388E−14 |
| RTFDC1 | 5.64E−25 | −0.294 | 0.436 | 0.184 | 5.493E−21 | KLF10 | 1.43E−18 | 0.287 | 0.348 | 0.123 | 1.396E−14 |
| PQBP1 | 5.76E−25 | −0.366 | 0.375 | 0.158 | 5.612E−21 | ATP11B | 1.51E−18 | 0.300 | 0.383 | 0.136 | 1.475E−14 |
| ATP5F1 | 6.62E−25 | −0.566 | 0.363 | 0.154 | 6.446E−21 | BOLA3 | 1.54E−18 | 0.385 | 0.29 | 0.057 | 1.503E−14 |
| ATG12 | 7.05E−25 | −0.590 | 0.318 | 0.105 | 6.874E−21 | METTL5 | 1.55E−18 | 0.531 | 0.352 | 0.092 | 1.511E−14 |
| FLOT2 | 7.08E−25 | −1.028 | 0.203 | 0.079 | 6.896E−21 | RPP38 | 1.55E−18 | 0.320 | 0.487 | 0.215 | 1.514E−14 |
| MT-TF | 7.62E−25 | −0.260 | 0.218 | 0.026 | 7.429E−21 | C12orf29 | 1.60E−18 | 0.684 | 0.292 | 0.066 | 1.556E−14 |
| DDA1 | 8.44E−25 | −0.442 | 0.337 | 0.114 | 8.222E−21 | ERO1L | 1.67E−18 | 0.801 | 0.286 | 0.044 | 1.631E−14 |
| CD14 | 9.19E−25 | −0.644 | 0.345 | 0.149 | 8.954E−21 | TOM1L1 | 1.72E−18 | 0.364 | 0.286 | 0.057 | 1.673E−14 |
| SRPRB | 1.01E−24 | −0.300 | 0.378 | 0.149 | 9.846E−21 | PARD3 | 1.73E−18 | 0.358 | 0.348 | 0.105 | 1.683E−14 |
| FAM96A | 1.05E−24 | −0.378 | 0.404 | 0.171 | 1.028E−20 | GOLGA7 | 1.78E−18 | 0.297 | 0.286 | 0.07 | 1.734E−14 |
| ZDHHC12 | 1.15E−24 | −0.931 | 0.218 | 0.075 | 1.117E−20 | CPNE1 | 1.84E−18 | 0.375 | 0.286 | 0.061 | 1.797E−14 |
| ISCA1 | 1.19E−24 | −0.749 | 0.213 | 0.061 | 1.156E−20 | FAM210B | 1.87E−18 | 0.638 | 0.298 | 0.061 | 1.822E−14 |
| STOM | 1.25E−24 | −0.312 | 0.431 | 0.197 | 1.222E−20 | PGM3 | 1.99E−18 | 0.889 | 0.307 | 0.057 | 1.939E−14 |
| NDUFA8 | 1.44E−24 | −0.475 | 0.34 | 0.14 | 1.401E−20 | LUC7L | 2.09E−18 | 0.365 | 0.309 | 0.075 | 2.035E−14 |
| POLR2H | 1.97E−24 | −0.442 | 0.357 | 0.14 | 1.923E−20 | IFT20 | 2.12E−18 | 0.389 | 0.345 | 0.105 | 2.061E−14 |
| BCLAF1 | 2.13E−24 | −0.497 | 0.407 | 0.189 | 2.073E−20 | JOSD1 | 2.12E−18 | 0.457 | 0.286 | 0.053 | 2.063E−14 |
| NSD1 | 2.14E−24 | −0.379 | 0.448 | 0.211 | 2.087E−20 | CTNNAL1 | 2.14E−18 | 0.840 | 0.337 | 0.075 | 2.085E−14 |
| CASP6 | 2.18E−24 | −0.273 | 0.39 | 0.118 | 2.129E−20 | NUCKS1 | 2.24E−18 | 0.265 | 0.995 | 0.864 | 2.181E−14 |
| ALYREF | 2.20E−24 | −0.381 | 0.239 | 0.044 | 2.148E−20 | SEC11A | 2.24E−18 | 0.599 | 0.356 | 0.088 | 2.187E−14 |
| CCDC167 | 2.63E−24 | −0.375 | 0.381 | 0.14 | 2.560E−20 | FAM46A | 2.33E−18 | 0.875 | 0.396 | 0.123 | 2.268E−14 |
| SMIM7 | 2.90E−24 | −0.734 | 0.248 | 0.096 | 2.826E−20 | RIOK1 | 2.34E−18 | 0.265 | 0.309 | 0.079 | 2.285E−14 |
| MIR4435- | 2.99E−24 | −1.431 | 0.224 | 0.114 | 2.910E−20 | ATAD1 | 2.41E−18 | 0.257 | 0.263 | 0.044 | 2.344E−14 |
| 1HG | | | | | | | | | | | |
| RPIA | 2.99E−24 | −0.273 | 0.286 | 0.075 | 2.912E−20 | TXNRD1 | 2.42E−18 | 0.376 | 0.421 | 0.158 | 2.355E−14 |
| COX19 | 3.06E−24 | −0.370 | 0.321 | 0.114 | 2.983E−20 | SPG20 | 2.47E−18 | 0.391 | 0.351 | 0.101 | 2.403E−14 |
| RRP7A | 3.11E−24 | −0.576 | 0.312 | 0.105 | 3.028E−20 | SLC39A10 | 2.59E−18 | 0.583 | 0.277 | 0.053 | 2.528E−14 |
| IFITM2 | 3.14E−24 | −1.199 | 0.26 | 0.158 | 3.063E−20 | FUBP1 | 2.85E−18 | 0.408 | 0.383 | 0.123 | 2.775E−14 |
| DANCR | 3.20E−24 | −0.631 | 0.298 | 0.123 | 3.117E−20 | ATP2A2 | 3.12E−18 | 0.596 | 0.304 | 0.07 | 3.045E−14 |
| SLC25A1 | 3.54E−24 | −1.076 | 0.168 | 0.079 | 3.447E−20 | RNF115 | 3.32E−18 | 0.656 | 0.351 | 0.092 | 3.237E−14 |
| PRPF19 | 4.08E−24 | −0.584 | 0.298 | 0.088 | 3.976E−20 | UBA2 | 3.37E−18 | 0.451 | 0.307 | 0.066 | 3.286E−14 |
| CDC37 | 4.22E−24 | −0.271 | 0.365 | 0.11 | 4.110E−20 | DNAJC15 | 3.64E−18 | 0.253 | 0.313 | 0.092 | 3.546E−14 |
| SREBF2 | 4.55E−24 | −0.314 | 0.349 | 0.101 | 4.431E−20 | LMBRD1 | 4.17E−18 | 0.910 | 0.325 | 0.066 | 4.068E−14 |
| DAXX | 4.91E−24 | −0.299 | 0.345 | 0.118 | 4.782E−20 | GRSF1 | 4.30E−18 | 0.477 | 0.378 | 0.114 | 4.194E−14 |
| NDUFV2 | 5.13E−24 | −1.219 | 0.159 | 0.066 | 4.998E−20 | HIST1H1E | 4.38E−18 | 0.950 | 0.343 | 0.07 | 4.269E−14 |
| APOPT1 | 5.79E−24 | −0.318 | 0.336 | 0.114 | 5.646E−20 | FAM105B | 4.41E−18 | 0.895 | 0.263 | 0.031 | 4.295E−14 |
| ETV3 | 6.81E−24 | −0.436 | 0.307 | 0.132 | 6.638E−20 | UBE2B | 4.83E−18 | 0.450 | 0.301 | 0.075 | 4.703E−14 |
| PVRL2 | 6.84E−24 | −1.070 | 0.172 | 0.07 | 6.670E−20 | ICK | 4.87E−18 | 0.711 | 0.363 | 0.105 | 4.744E−14 |
| ARHGAP21 | 7.26E−24 | −0.518 | 0.33 | 0.123 | 7.078E−20 | NUDT9 | 4.91E−18 | 0.703 | 0.275 | 0.044 | 4.783E−14 |
| SLC38A2 | 8.11E−24 | −0.328 | 0.46 | 0.241 | 7.902E−20 | POR | 5.06E−18 | 0.294 | 0.287 | 0.075 | 4.927E−14 |
| SLC2A1 | 1.04E−23 | −0.883 | 0.218 | 0.101 | 1.009E−19 | NEK7 | 5.86E−18 | 0.421 | 0.337 | 0.11 | 5.711E−14 |
| DDX18 | 1.46E−23 | −0.387 | 0.411 | 0.206 | 1.427E−19 | UTP18 | 5.90E−18 | 0.533 | 0.313 | 0.07 | 5.747E−14 |
| STOML2 | 1.50E−23 | −0.257 | 0.343 | 0.118 | 1.465E−19 | PPIL1 | 6.07E−18 | 0.693 | 0.256 | 0.031 | 5.913E−14 |
| UBAC2 | 1.52E−23 | −0.472 | 0.295 | 0.105 | 1.483E−19 | HIST2H2AC | 6.44E−18 | 0.314 | 0.343 | 0.11 | 6.280E−14 |
| UBA1 | 1.53E−23 | −0.584 | 0.33 | 0.136 | 1.487E−19 | USP16 | 6.50E−18 | 0.595 | 0.357 | 0.105 | 6.330E−14 |
| SLC31A1 | 1.53E−23 | −0.433 | 0.303 | 0.123 | 1.490E−19 | EIF1B | 6.53E−18 | 0.501 | 0.319 | 0.092 | 6.361E−14 |
| PDXDC1 | 1.57E−23 | −0.340 | 0.348 | 0.132 | 1.527E−19 | DCBLD2 | 7.02E−18 | 0.650 | 0.309 | 0.07 | 6.836E−14 |
| MMP14 | 1.58E−23 | −0.663 | 0.248 | 0.075 | 1.542E−19 | MCFD2 | 7.06E−18 | 0.585 | 0.365 | 0.127 | 6.877E−14 |
| EPN1 | 1.62E−23 | −0.514 | 0.25 | 0.075 | 1.576E−19 | HIST1H1D | 7.09E−18 | 0.693 | 0.309 | 0.118 | 6.910E−14 |
| AL592183.1 | 1.84E−23 | −0.499 | 0.284 | 0.088 | 1.791E−19 | ARL1 | 7.60E−18 | 0.941 | 0.368 | 0.105 | 7.403E−14 |
| MRPL54 | 1.98E−23 | −0.535 | 0.215 | 0.053 | 1.929E−19 | AVL9 | 7.62E−18 | 0.383 | 0.331 | 0.114 | 7.430E−14 |
| RPL39L | 2.01E−23 | −0.881 | 0.351 | 0.158 | 1.954E−19 | UGGT2 | 7.77E−18 | 0.586 | 0.325 | 0.092 | 7.567E−14 |
| ATP1B3 | 2.55E−23 | −0.547 | 0.331 | 0.136 | 2.487E−19 | ITGB3BP | 8.38E−18 | 0.381 | 0.297 | 0.07 | 8.163E−14 |
| PSMD13 | 2.72E−23 | −0.279 | 0.324 | 0.092 | 2.654E−19 | CCNB1IP1 | 9.59E−18 | 0.676 | 0.266 | 0.039 | 9.349E−14 |
| HINT2 | 2.77E−23 | −0.651 | 0.268 | 0.088 | 2.698E−19 | HINT3 | 9.64E−18 | 0.478 | 0.324 | 0.083 | 9.392E−14 |
| CS | 2.81E−23 | −0.777 | 0.241 | 0.105 | 2.738E−19 | STT3B | 9.86E−18 | 0.537 | 0.334 | 0.092 | 9.608E−14 |
| C18orf32 | 2.81E−23 | −1.106 | 0.186 | 0.053 | 2.741E−19 | ARL8B | 1.01E−17 | 0.686 | 0.351 | 0.088 | 9.885E−14 |
| PRPF4B | 2.84E−23 | −0.295 | 0.419 | 0.18 | 2.771E−19 | PTRH2 | 1.08E−17 | 0.338 | 0.263 | 0.061 | 1.048E−13 |
| METRN | 3.32E−23 | −0.661 | 0.163 | 0.039 | 3.234E−19 | ST3GAL6 | 1.10E−17 | 0.373 | 0.322 | 0.092 | 1.068E−13 |
| ADI1 | 3.45E−23 | −0.439 | 0.313 | 0.123 | 3.366E−19 | NAPG | 1.18E−17 | 0.321 | 0.298 | 0.075 | 1.145E−13 |
| CKLF | 3.55E−23 | −0.401 | 0.331 | 0.127 | 3.457E−19 | RBM23 | 1.22E−17 | 0.343 | 0.36 | 0.14 | 1.192E−13 |
| KLHDC3 | 4.02E−23 | −0.312 | 0.349 | 0.123 | 3.921E−19 | USP6NL | 1.56E−17 | 0.495 | 0.295 | 0.061 | 1.517E−13 |
| LDLR | 4.38E−23 | −0.539 | 0.315 | 0.11 | 4.267E−19 | RRM1 | 1.78E−17 | 0.629 | 0.289 | 0.066 | 1.730E−13 |
| CNPY2 | 5.00E−23 | −0.587 | 0.363 | 0.171 | 4.871E−19 | HAT1 | 1.78E−17 | 0.253 | 0.262 | 0.048 | 1.733E−13 |
| ARL6IP1 | 5.10E−23 | −0.336 | 0.641 | 0.465 | 4.967E−19 | DDX60 | 1.82E−17 | 0.573 | 0.352 | 0.101 | 1.777E−13 |
| PCYOX1 | 5.44E−23 | −0.519 | 0.365 | 0.171 | 5.300E−19 | SLTM | 1.89E−17 | 0.556 | 0.36 | 0.127 | 1.845E−13 |
| TCEA1 | 5.45E−23 | −1.109 | 0.192 | 0.101 | 5.312E−19 | MAP7D1 | 1.99E−17 | 0.606 | 0.303 | 0.083 | 1.939E−13 |
| CITED4 | 5.77E−23 | −0.630 | 0.238 | 0.083 | 5.619E−19 | TJP1 | 2.04E−17 | 0.489 | 0.313 | 0.083 | 1.987E−13 |
| VPS28 | 6.28E−23 | −0.304 | 0.371 | 0.171 | 6.117E−19 | PARP4 | 2.18E−17 | 0.461 | 0.33 | 0.083 | 2.121E−13 |
| SCAMP3 | 6.51E−23 | −0.641 | 0.269 | 0.127 | 6.341E−19 | PSMG1 | 2.40E−17 | 0.791 | 0.284 | 0.061 | 2.337E−13 |
| PRKCI | 7.06E−23 | −0.377 | 0.36 | 0.158 | 6.882E−19 | IL13RA1 | 2.57E−17 | 0.442 | 0.319 | 0.096 | 2.507E−13 |
| ISG20L2 | 8.25E−23 | −0.273 | 0.366 | 0.118 | 8.037E−19 | IFIT1 | 2.62E−17 | 0.445 | 0.372 | 0.149 | 2.550E−13 |
| SQRDL | 8.38E−23 | −0.557 | 0.26 | 0.083 | 8.168E−19 | ARF1 | 2.64E−17 | 0.396 | 0.961 | 0.798 | 2.573E−13 |
| HEXB | 9.14E−23 | −0.369 | 0.375 | 0.158 | 8.907E−19 | C3orf38 | 2.98E−17 | 1.006 | 0.333 | 0.07 | 2.901E−13 |
| UPF3A | 9.55E−23 | −0.598 | 0.247 | 0.083 | 9.310E−19 | GGNBP2 | 3.98E−17 | 0.398 | 0.313 | 0.092 | 3.875E−13 |
| UROD | 9.59E−23 | −0.430 | 0.327 | 0.193 | 9.343E−19 | ARV1 | 4.05E−17 | 0.544 | 0.331 | 0.079 | 3.944E−13 |
| CDK11B | 9.96E−23 | −1.098 | 0.166 | 0.075 | 9.705E−19 | MESDC2 | 4.22E−17 | 0.441 | 0.274 | 0.057 | 4.108E−13 |
| HSPA1A | 1.03E−22 | −0.320 | 0.307 | 0.096 | 9.994E−19 | ATP2C2 | 4.52E−17 | 0.643 | 0.287 | 0.053 | 4.406E−13 |
| KHSRP | 1.03E−22 | −0.715 | 0.295 | 0.127 | 1.008E−18 | CNOT7 | 4.60E−17 | 0.271 | 0.281 | 0.066 | 4.482E−13 |
| PSMB9 | 1.05E−22 | −0.495 | 0.31 | 0.149 | 1.023E−18 | PMP22 | 4.66E−17 | 1.220 | 0.342 | 0.101 | 4.540E−13 |
| SUMF2 | 1.12E−22 | −0.764 | 0.247 | 0.114 | 1.096E−18 | GMNN | 4.72E−17 | 0.449 | 0.301 | 0.07 | 4.602E−13 |
| TUBA1A | 1.44E−22 | −0.467 | 0.396 | 0.202 | 1.402E−18 | PPP6R2 | 5.04E−17 | 0.251 | 0.3 | 0.092 | 4.913E−13 |
| MRPL50 | 1.49E−22 | −0.530 | 0.339 | 0.114 | 1.451E−18 | RAB9A | 5.23E−17 | 0.381 | 0.259 | 0.048 | 5.094E−13 |
| TAF9 | 1.63E−22 | −0.253 | 0.392 | 0.145 | 1.585E−18 | USP9X | 5.27E−17 | 0.482 | 0.327 | 0.096 | 5.133E−13 |
| ELOVL1 | 1.76E−22 | −0.614 | 0.251 | 0.079 | 1.718E−18 | SRP54 | 5.62E−17 | 1.165 | 0.312 | 0.066 | 5.477E−13 |
| MYCBP | 1.83E−22 | −0.973 | 0.204 | 0.066 | 1.786E−18 | HBS1L | 6.31E−17 | 0.425 | 0.316 | 0.079 | 6.152E−13 |
| SULF2 | 1.88E−22 | −0.478 | 0.327 | 0.132 | 1.834E−18 | C3orf58 | 6.64E−17 | 0.337 | 0.321 | 0.096 | 6.476E−13 |
| ANXA11 | 2.06E−22 | −0.516 | 0.277 | 0.101 | 2.006E−18 | ITGA2 | 6.78E−17 | 0.965 | 0.303 | 0.061 | 6.605E−13 |
| FAHD2A | 2.10E−22 | −0.572 | 0.228 | 0.057 | 2.046E−18 | MAGT1 | 6.86E−17 | 0.498 | 0.334 | 0.096 | 6.685E−13 |
| SRF | 3.07E−22 | −0.695 | 0.189 | 0.061 | 2.994E−18 | STX7 | 7.55E−17 | 0.645 | 0.352 | 0.096 | 7.357E−13 |
| HIST1H4H | 3.12E−22 | −1.218 | 0.18 | 0.136 | 3.036E−18 | LYPLAL1 | 7.71E−17 | 0.593 | 0.363 | 0.127 | 7.513E−13 |
| NDUFV3 | 3.19E−22 | −0.265 | 0.297 | 0.083 | 3.113E−18 | BDH2 | 8.41E−17 | 0.355 | 0.245 | 0.044 | 8.196E−13 |
| TOP1 | 3.22E−22 | −0.934 | 0.236 | 0.101 | 3.142E−18 | TMEM160 | 8.47E−17 | 0.854 | 0.271 | 0.048 | 8.249E−13 |
| PSMC1 | 3.41E−22 | −0.986 | 0.133 | 0.048 | 3.321E−18 | COMMD8 | 8.91E−17 | 0.621 | 0.368 | 0.123 | 8.680E−13 |
| NTMT1 | 3.47E−22 | −0.533 | 0.231 | 0.083 | 3.385E−18 | MRPL42 | 9.01E−17 | 0.568 | 0.362 | 0.105 | 8.781E−13 |
| TFPT | 3.61E−22 | −1.002 | 0.192 | 0.066 | 3.514E−18 | ILKAP | 9.55E−17 | 0.490 | 0.315 | 0.075 | 9.309E−13 |
| LYZ | 3.96E−22 | −2.412 | 0.086 | 0.184 | 3.855E−18 | ZDHHC6 | 9.66E−17 | 0.650 | 0.283 | 0.061 | 9.414E−13 |
| ICA1 | 4.27E−22 | −0.278 | 0.331 | 0.14 | 4.162E−18 | PIGS | 9.78E−17 | 0.661 | 0.334 | 0.083 | 9.530E−13 |
| BABAM1 | 4.71E−22 | −0.536 | 0.239 | 0.066 | 4.589E−18 | RTP4 | 1.00E−16 | 0.336 | 0.284 | 0.061 | 9.785E−13 |
| SEC61A1 | 4.79E−22 | −0.434 | 0.301 | 0.14 | 4.670E−18 | MRPS33 | 1.11E−16 | 0.252 | 0.331 | 0.11 | 1.082E−12 |
| UBE2E3 | 4.98E−22 | −1.018 | 0.221 | 0.096 | 4.849E−18 | DOCK1 | 1.25E−16 | 1.063 | 0.284 | 0.053 | 1.222E−12 |
| HSPB11 | 5.11E−22 | −0.343 | 0.369 | 0.154 | 4.977E−18 | MMGT1 | 1.30E−16 | 0.289 | 0.343 | 0.127 | 1.268E−12 |
| CHCHD3 | 5.20E−22 | −0.467 | 0.327 | 0.136 | 5.067E−18 | MT-TT | 1.34E−16 | 0.530 | 0.188 | 0.004 | 1.302E−12 |
| RPS13 | 5.59E−22 | −0.308 | 0.971 | 0.838 | 5.444E−18 | ARMCX2 | 1.44E−16 | 0.268 | 0.277 | 0.066 | 1.402E−12 |
| GRINA | 5.85E−22 | −0.825 | 0.18 | 0.053 | 5.704E−18 | ZNF639 | 1.45E−16 | 0.420 | 0.292 | 0.066 | 1.417E−12 |
| LINC00657 | 5.88E−22 | −0.799 | 0.277 | 0.145 | 5.728E−18 | LTBP1 | 1.51E−16 | 0.283 | 0.3 | 0.092 | 1.470E−12 |
| CTTN | 5.92E−22 | −0.552 | 0.33 | 0.158 | 5.769E−18 | GART | 1.63E−16 | 0.609 | 0.29 | 0.057 | 1.591E−12 |
| SCO2 | 5.95E−22 | −0.306 | 0.301 | 0.096 | 5.802E−18 | HMGXB4 | 1.66E−16 | 0.760 | 0.259 | 0.048 | 1.622E−12 |
| MRPL37 | 8.59E−22 | −0.319 | 0.325 | 0.118 | 8.366E−18 | PIK3CA | 1.71E−16 | 0.269 | 0.293 | 0.079 | 1.669E−12 |
| CHCHD5 | 9.04E−22 | −1.040 | 0.148 | 0.066 | 8.807E−18 | GCNT2 | 1.91E−16 | 0.424 | 0.312 | 0.083 | 1.858E−12 |
| DGUOK | 9.06E−22 | −0.340 | 0.363 | 0.127 | 8.833E−18 | UBE2A | 1.96E−16 | 0.274 | 0.295 | 0.088 | 1.912E−12 |
| ZBTB38 | 9.37E−22 | −0.266 | 0.427 | 0.228 | 9.132E−18 | MED8 | 2.01E−16 | 0.906 | 0.259 | 0.035 | 1.960E−12 |
| ARPP19 | 9.90E−22 | −0.533 | 0.339 | 0.14 | 9.646E−18 | FSTL1 | 2.27E−16 | 0.556 | 0.256 | 0.07 | 2.212E−12 |
| ZFP36L2 | 1.03E−21 | −0.692 | 0.354 | 0.18 | 1.007E−17 | SESTD1 | 2.38E−16 | 0.470 | 0.307 | 0.088 | 2.324E−12 |
| APOE | 1.15E−21 | −0.919 | 0.242 | 0.118 | 1.116E−17 | ANGEL2 | 2.46E−16 | 0.275 | 0.303 | 0.096 | 2.394E−12 |
| KPNA2 | 1.16E−21 | −0.845 | 0.322 | 0.25 | 1.129E−17 | KDM3A | 2.59E−16 | 1.052 | 0.247 | 0.031 | 2.524E−12 |
| IGF2BP2 | 1.17E−21 | −0.532 | 0.328 | 0.14 | 1.141E−17 | TBC1D15 | 2.62E−16 | 0.627 | 0.315 | 0.079 | 2.555E−12 |
| SF3B4 | 1.26E−21 | −0.789 | 0.242 | 0.083 | 1.231E−17 | ACOT13 | 2.92E−16 | 0.590 | 0.28 | 0.053 | 2.850E−12 |
| ODF3B | 1.31E−21 | −0.765 | 0.253 | 0.101 | 1.277E−17 | PUM1 | 3.02E−16 | 0.347 | 0.34 | 0.11 | 2.947E−12 |
| C1orf85 | 1.37E−21 | −0.679 | 0.236 | 0.092 | 1.331E−17 | PIK3C2A | 3.04E−16 | 0.545 | 0.369 | 0.127 | 2.961E−12 |
| CYB561 | 1.46E−21 | −0.684 | 0.295 | 0.127 | 1.423E−17 | CD109 | 3.39E−16 | 0.361 | 0.309 | 0.114 | 3.302E−12 |
| PGLS | 1.57E−21 | −0.775 | 0.275 | 0.132 | 1.528E−17 | GLG1 | 3.44E−16 | 0.371 | 0.324 | 0.101 | 3.357E−12 |
| CHID1 | 1.67E−21 | −0.330 | 0.283 | 0.11 | 1.624E−17 | BOLA1 | 3.51E−16 | 0.301 | 0.271 | 0.07 | 3.420E−12 |
| MRPL27 | 1.69E−21 | −0.511 | 0.28 | 0.083 | 1.648E−17 | NFYB | 3.60E−16 | 0.720 | 0.272 | 0.053 | 3.512E−12 |
| SLCO3A1 | 1.72E−21 | −0.507 | 0.292 | 0.11 | 1.673E−17 | UBAP2 | 3.85E−16 | 1.040 | 0.277 | 0.044 | 3.750E−12 |
| REXO4 | 1.76E−21 | −0.346 | 0.28 | 0.088 | 1.710E−17 | TIMM21 | 3.98E−16 | 0.438 | 0.287 | 0.083 | 3.882E−12 |
| PGD | 1.83E−21 | −0.556 | 0.295 | 0.092 | 1.782E−17 | PHF6 | 4.13E−16 | 0.839 | 0.287 | 0.057 | 4.026E−12 |
| FN3KRP | 2.01E−21 | −0.573 | 0.283 | 0.123 | 1.955E−17 | PHF5A | 5.34E−16 | 0.401 | 0.333 | 0.096 | 5.207E−12 |
| NUP50 | 2.03E−21 | −0.475 | 0.298 | 0.123 | 1.978E−17 | CTSH | 5.39E−16 | 0.803 | 0.269 | 0.057 | 5.255E−12 |
| JUP | 2.30E−21 | −0.690 | 0.356 | 0.167 | 2.244E−17 | RBBP8 | 5.57E−16 | 0.295 | 0.307 | 0.101 | 5.426E−12 |
| LLPH | 2.31E−21 | −0.685 | 0.284 | 0.127 | 2.250E−17 | ARMCX1 | 6.85E−16 | 1.021 | 0.265 | 0.039 | 6.679E−12 |
| COMT | 2.50E−21 | −0.300 | 0.381 | 0.14 | 2.434E−17 | MFSD6 | 7.42E−16 | 0.446 | 0.307 | 0.088 | 7.234E−12 |
| METTL2A | 2.60E−21 | −0.765 | 0.23 | 0.101 | 2.531E−17 | TTC3 | 7.53E−16 | 0.252 | 0.334 | 0.114 | 7.339E−12 |
| FBXW5 | 2.91E−21 | −0.256 | 0.333 | 0.127 | 2.834E−17 | GMDS | 7.92E−16 | 1.005 | 0.225 | 0.031 | 7.715E−12 |
| TCTEX1D2 | 3.07E−21 | −0.985 | 0.2 | 0.075 | 2.995E−17 | TFDP2 | 8.89E−16 | 0.861 | 0.293 | 0.061 | 8.665E−12 |
| H1F0 | 3.15E−21 | −0.363 | 0.424 | 0.241 | 3.069E−17 | KIAA1715 | 8.92E−16 | 0.891 | 0.269 | 0.053 | 8.694E−12 |
| NDUFV1 | 3.24E−21 | −0.741 | 0.245 | 0.07 | 3.162E−17 | DHX36 | 1.13E−15 | 0.404 | 0.39 | 0.167 | 1.106E−11 |
| CDK2AP1 | 3.65E−21 | −0.737 | 0.21 | 0.053 | 3.559E−17 | MAT2A | 1.17E−15 | 0.262 | 0.248 | 0.053 | 1.138E−11 |
| FCF1 | 4.54E−21 | −0.677 | 0.228 | 0.07 | 4.424E−17 | SREK1 | 1.20E−15 | 0.469 | 0.269 | 0.07 | 1.169E−11 |
| PFKL | 5.04E−21 | −0.549 | 0.278 | 0.132 | 4.914E−17 | SMNDC1 | 1.22E−15 | 0.958 | 0.28 | 0.061 | 1.189E−11 |
| SMIM4 | 5.34E−21 | −0.632 | 0.224 | 0.075 | 5.208E−17 | RBM18 | 1.23E−15 | 0.466 | 0.295 | 0.101 | 1.201E−11 |
| LYRM2 | 5.58E−21 | −0.274 | 0.424 | 0.219 | 5.435E−17 | GNPDA1 | 1.25E−15 | 0.291 | 0.272 | 0.079 | 1.214E−11 |
| DAG1 | 5.66E−21 | −0.401 | 0.298 | 0.11 | 5.519E−17 | DYNC1LI2 | 1.28E−15 | 0.331 | 0.352 | 0.127 | 1.245E−11 |
| ETFB | 5.98E−21 | −0.565 | 0.33 | 0.123 | 5.823E−17 | STX17 | 1.30E−15 | 0.651 | 0.251 | 0.048 | 1.264E−11 |
| MYL6B | 6.19E−21 | −0.723 | 0.234 | 0.07 | 6.031E−17 | S100A6 | 1.38E−15 | 0.297 | 0.968 | 0.877 | 1.348E−11 |
| COMMD1 | 6.57E−21 | −0.425 | 0.274 | 0.127 | 6.405E−17 | UBA3 | 1.78E−15 | 0.534 | 0.315 | 0.105 | 1.734E−11 |
| PLIN3 | 7.15E−21 | −0.566 | 0.234 | 0.101 | 6.969E−17 | POLE4 | 1.89E−15 | 0.263 | 0.257 | 0.075 | 1.838E−11 |
| PUF60 | 7.27E−21 | −0.390 | 0.309 | 0.101 | 7.089E−17 | SLC25A37 | 1.93E−15 | 0.579 | 0.313 | 0.083 | 1.884E−11 |
| MEA1 | 7.56E−21 | −0.703 | 0.278 | 0.123 | 7.366E−17 | KIF1B | 1.96E−15 | 0.362 | 0.269 | 0.066 | 1.910E−11 |
| RNF145 | 7.92E−21 | −0.503 | 0.277 | 0.118 | 7.720E−17 | RNF2 | 2.03E−15 | 0.350 | 0.269 | 0.07 | 1.983E−11 |
| RPS29 | 8.11E−21 | −0.585 | 0.983 | 0.89 | 7.899E−17 | SEC22B | 2.31E−15 | 0.670 | 0.316 | 0.105 | 2.254E−11 |
| C21orf59 | 8.20E−21 | −0.413 | 0.239 | 0.079 | 7.989E−17 | SMIM15 | 2.37E−15 | 0.779 | 0.351 | 0.101 | 2.308E−11 |
| ZNF148 | 8.37E−21 | −0.285 | 0.363 | 0.154 | 8.156E−17 | ATL3 | 2.43E−15 | 0.395 | 0.295 | 0.105 | 2.367E−11 |
| TIMM50 | 8.45E−21 | −0.556 | 0.274 | 0.11 | 8.238E−17 | SUZ12 | 2.54E−15 | 0.437 | 0.322 | 0.105 | 2.476E−11 |
| CANT1 | 9.16E−21 | −0.388 | 0.301 | 0.105 | 8.930E−17 | KDM5B | 2.65E−15 | 0.427 | 0.346 | 0.114 | 2.578E−11 |
| CHD2 | 1.09E−20 | −0.324 | 0.395 | 0.189 | 1.058E−16 | NAT10 | 2.67E−15 | 0.290 | 0.298 | 0.079 | 2.604E−11 |
| UBE2M | 1.10E−20 | −1.245 | 0.106 | 0.066 | 1.076E−16 | NUDT3 | 2.71E−15 | 0.280 | 0.225 | 0.048 | 2.645E−11 |
| YIPF3 | 1.11E−20 | −0.466 | 0.262 | 0.079 | 1.079E−16 | SFSWAP | 2.72E−15 | 0.636 | 0.247 | 0.048 | 2.654E−11 |
| MRPL1 | 1.13E−20 | −0.409 | 0.354 | 0.158 | 1.098E−16 | CETN3 | 2.86E−15 | 0.419 | 0.215 | 0.039 | 2.790E−11 |
| DAZAP2 | 1.27E−20 | −0.328 | 0.306 | 0.118 | 1.234E−16 | NUDT15 | 3.08E−15 | 0.809 | 0.227 | 0.026 | 3.005E−11 |
| ARRDC1 | 1.29E−20 | −0.791 | 0.228 | 0.105 | 1.254E−16 | TCF12 | 3.31E−15 | 0.690 | 0.331 | 0.114 | 3.230E−11 |
| UQCRC2 | 1.35E−20 | −0.391 | 0.3 | 0.088 | 1.313E−16 | WDR33 | 3.32E−15 | 0.341 | 0.28 | 0.07 | 3.238E−11 |
| CAPZA1 | 1.35E−20 | −0.277 | 0.386 | 0.175 | 1.315E−16 | MTMR12 | 3.33E−15 | 0.472 | 0.328 | 0.118 | 3.248E−11 |
| MCMBP | 1.43E−20 | −0.572 | 0.272 | 0.083 | 1.396E−16 | DCBLD1 | 3.55E−15 | 0.784 | 0.133 | 0.013 | 3.462E−11 |
| RCC2 | 1.45E−20 | −0.449 | 0.289 | 0.105 | 1.409E−16 | ANKLE2 | 3.65E−15 | 0.503 | 0.254 | 0.053 | 3.561E−11 |
| GOT2 | 1.59E−20 | −0.636 | 0.343 | 0.149 | 1.547E−16 | B3GALNT1 | 3.88E−15 | 0.397 | 0.286 | 0.083 | 3.779E−11 |
| PLS3 | 1.91E−20 | −0.425 | 0.33 | 0.114 | 1.863E−16 | C16orf80 | 4.15E−15 | 0.496 | 0.265 | 0.066 | 4.041E−11 |
| PDHA1 | 1.93E−20 | −0.446 | 0.352 | 0.154 | 1.878E−16 | NUPL2 | 4.16E−15 | 0.291 | 0.268 | 0.075 | 4.056E−11 |
| NOP56 | 1.96E−20 | −0.318 | 0.331 | 0.118 | 1.908E−16 | GPX8 | 4.49E−15 | 0.365 | 0.266 | 0.066 | 4.378E−11 |
| C6orf62 | 2.15E−20 | −0.328 | 0.362 | 0.171 | 2.096E−16 | UBA6 | 4.66E−15 | 0.330 | 0.262 | 0.066 | 4.537E−11 |
| NRBF2 | 2.20E−20 | −0.516 | 0.204 | 0.048 | 2.145E−16 | CUL3 | 5.44E−15 | 0.371 | 0.262 | 0.075 | 5.299E−11 |
| FAHD1 | 2.22E−20 | −0.344 | 0.231 | 0.057 | 2.161E−16 | HSDL2 | 5.75E−15 | 0.423 | 0.216 | 0.039 | 5.603E−11 |
| STK25 | 2.65E−20 | −1.077 | 0.204 | 0.101 | 2.580E−16 | HIF1A | 5.85E−15 | 0.297 | 0.303 | 0.101 | 5.704E−11 |
| RYBP | 2.91E−20 | −0.298 | 0.331 | 0.101 | 2.836E−16 | EFNA1 | 5.86E−15 | 0.253 | 0.298 | 0.096 | 5.709E−11 |
| GUSB | 2.92E−20 | −0.532 | 0.275 | 0.118 | 2.842E−16 | GINM1 | 5.91E−15 | 1.091 | 0.253 | 0.044 | 5.756E−11 |
| TAX1BP3 | 3.43E−20 | −0.905 | 0.151 | 0.044 | 3.342E−16 | PTPLB | 5.97E−15 | 0.356 | 0.318 | 0.105 | 5.813E−11 |
| SPTSSA | 3.46E−20 | −0.332 | 0.411 | 0.175 | 3.367E−16 | KCMF1 | 6.19E−15 | 0.520 | 0.283 | 0.07 | 6.028E−11 |
| TMEM219 | 4.93E−20 | −0.667 | 0.212 | 0.061 | 4.806E−16 | DCTN2 | 6.32E−15 | 0.422 | 0.262 | 0.061 | 6.164E−11 |
| S100A8 | 5.25E−20 | −1.801 | 0.074 | 0.132 | 5.117E−16 | WDR41 | 6.56E−15 | 0.956 | 0.256 | 0.048 | 6.391E−11 |
| LIMS1 | 5.92E−20 | −0.669 | 0.28 | 0.118 | 5.770E−16 | FAT1 | 6.72E−15 | 0.336 | 0.259 | 0.092 | 6.551E−11 |
| BCL7C | 6.71E−20 | −0.619 | 0.197 | 0.048 | 6.542E−16 | SMEK1 | 6.98E−15 | 0.583 | 0.254 | 0.061 | 6.804E−11 |
| OTUB1 | 6.87E−20 | −0.506 | 0.234 | 0.079 | 6.695E−16 | ATF7IP | 7.03E−15 | 0.502 | 0.3 | 0.079 | 6.847E−11 |
| MYL9 | 7.09E−20 | −0.387 | 0.25 | 0.088 | 6.911E−16 | SYNM | 7.32E−15 | 0.616 | 0.257 | 0.07 | 7.130E−11 |
| MRPS16 | 7.29E−20 | −0.421 | 0.295 | 0.11 | 7.105E−16 | DHX15 | 8.15E−15 | 0.362 | 0.244 | 0.057 | 7.947E−11 |
| TMBIM1 | 7.47E−20 | −0.571 | 0.207 | 0.061 | 7.279E−16 | SNX27 | 8.17E−15 | 0.370 | 0.241 | 0.057 | 7.957E−11 |
| CNN2 | 7.57E−20 | −0.440 | 0.333 | 0.127 | 7.381E−16 | DCUN1D4 | 8.49E−15 | 0.375 | 0.371 | 0.136 | 8.273E−11 |
| FAM120A | 7.62E−20 | −0.320 | 0.396 | 0.206 | 7.426E−16 | TRIM24 | 1.01E−14 | 0.994 | 0.298 | 0.075 | 9.800E−11 |
| ALKBH7 | 7.95E−20 | −0.510 | 0.259 | 0.088 | 7.745E−16 | KIAA0947 | 1.01E−14 | 0.314 | 0.33 | 0.123 | 9.832E−11 |
| RP11- | 8.23E−20 | −0.723 | 0.145 | 0.053 | 8.024E−16 | IRS2 | 1.01E−14 | 0.609 | 0.259 | 0.057 | 9.846E−11 |
| 290D2.6 | | | | | | | | | | | |
| PSMB6 | 8.40E−20 | −0.425 | 0.374 | 0.224 | 8.189E−16 | C1GALT1 | 1.05E−14 | 0.261 | 0.342 | 0.132 | 1.021E−10 |
| TMSB10 | 8.74E−20 | −0.596 | 1 | 0.996 | 8.516E−16 | MSANTD3 | 1.18E−14 | 0.324 | 0.215 | 0.039 | 1.147E−10 |
| EFR3A | 8.81E−20 | −0.267 | 0.3 | 0.092 | 8.581E−16 | ADAM10 | 1.19E−14 | 0.355 | 0.306 | 0.105 | 1.163E−10 |
| CLSTN1 | 8.91E−20 | −0.614 | 0.298 | 0.105 | 8.680E−16 | BNIP3 | 1.32E−14 | 0.653 | 0.244 | 0.044 | 1.283E−10 |
| TXNDC9 | 9.47E−20 | −0.397 | 0.253 | 0.083 | 9.224E−16 | DYRK3 | 1.39E−14 | 0.689 | 0.242 | 0.048 | 1.354E−10 |
| ANKRD11 | 9.50E−20 | −0.644 | 0.354 | 0.189 | 9.258E−16 | UHRF2 | 1.47E−14 | 0.620 | 0.222 | 0.035 | 1.435E−10 |
| SMU1 | 9.60E−20 | −0.268 | 0.324 | 0.11 | 9.352E−16 | NUP54 | 1.52E−14 | 0.565 | 0.269 | 0.061 | 1.486E−10 |
| HDHD3 | 9.68E−20 | −0.690 | 0.218 | 0.061 | 9.429E−16 | FAM91A1 | 1.53E−14 | 0.356 | 0.253 | 0.066 | 1.487E−10 |
| INSIGI | 9.81E−20 | −0.299 | 0.349 | 0.158 | 9.557E−16 | BCAP29 | 1.66E−14 | 0.301 | 0.245 | 0.07 | 1.620E−10 |
| CDC42SE1 | 1.05E−19 | −0.439 | 0.312 | 0.149 | 1.027E−15 | DDX58 | 1.83E−14 | 0.265 | 0.331 | 0.145 | 1.782E−10 |
| RP11- | 1.23E−19 | −0.596 | 0.236 | 0.083 | 1.200E−15 | PPP1R15A | 1.88E−14 | 1.019 | 0.303 | 0.075 | 1.827E−10 |
| 349A22.5 | | | | | | | | | | | |
| ANKRD12 | 1.36E−19 | −0.626 | 0.3 | 0.171 | 1.323E−15 | PIBF1 | 2.08E−14 | 0.465 | 0.215 | 0.031 | 2.023E−10 |
| INIP | 1.36E−19 | −0.403 | 0.278 | 0.105 | 1.327E−15 | DHX8 | 2.36E−14 | 0.471 | 0.204 | 0.07 | 2.302E−10 |
| COA1 | 1.42E−19 | −0.431 | 0.251 | 0.066 | 1.385E−15 | PSMG4 | 2.39E−14 | 0.297 | 0.221 | 0.079 | 2.331E−10 |
| ITSN2 | 1.58E−19 | −0.610 | 0.325 | 0.154 | 1.545E−15 | RAB5A | 2.82E−14 | 0.284 | 0.303 | 0.11 | 2.747E−10 |
| ECHI | 1.60E−19 | −0.746 | 0.274 | 0.145 | 1.556E−15 | AZI2 | 2.89E−14 | 0.380 | 0.298 | 0.096 | 2.815E−10 |
| AHCY | 1.62E−19 | −0.294 | 0.256 | 0.079 | 1.575E−15 | IFIT5 | 3.00E−14 | 0.490 | 0.277 | 0.075 | 2.928E−10 |
| COPS7A | 1.69E−19 | −0.542 | 0.26 | 0.105 | 1.646E−15 | CUL2 | 3.02E−14 | 0.527 | 0.318 | 0.118 | 2.945E−10 |
| POLR2K | 1.87E−19 | −0.273 | 0.259 | 0.083 | 1.825E−15 | ZNF217 | 3.29E−14 | 0.558 | 0.292 | 0.079 | 3.210E−10 |
| RBBP6 | 1.94E−19 | −0.581 | 0.331 | 0.149 | 1.890E−15 | SECISBP2L | 3.33E−14 | 0.415 | 0.247 | 0.083 | 3.245E−10 |
| CKB | 1.95E−19 | −0.444 | 0.244 | 0.075 | 1.902E−15 | SAMD12 | 3.34E−14 | 0.453 | 0.277 | 0.083 | 3.256E−10 |
| KNOP1 | 2.00E−19 | −0.258 | 0.325 | 0.118 | 1.951E−15 | MAP3K2 | 3.45E−14 | 0.457 | 0.245 | 0.057 | 3.357E−10 |
| MZT2A | 2.05E−19 | −0.715 | 0.265 | 0.118 | 2.000E−15 | FAM206A | 3.50E−14 | 0.432 | 0.271 | 0.079 | 3.414E−10 |
| UCK2 | 2.08E−19 | −0.448 | 0.245 | 0.075 | 2.023E−15 | CPEB4 | 3.52E−14 | 0.303 | 0.262 | 0.075 | 3.427E−10 |
| ZKSCAN1 | 2.28E−19 | −0.558 | 0.242 | 0.123 | 2.224E−15 | PPP3CA | 3.60E−14 | 0.350 | 0.25 | 0.07 | 3.507E−10 |
| PIGT | 2.29E−19 | −0.673 | 0.239 | 0.075 | 2.234E−15 | ANKRD17 | 3.61E−14 | 0.346 | 0.28 | 0.07 | 3.517E−10 |
| TIMM44 | 2.42E−19 | −0.335 | 0.287 | 0.101 | 2.359E−15 | FANCI | 3.72E−14 | 0.391 | 0.356 | 0.14 | 3.621E−10 |
| DDR1 | 2.55E−19 | −0.342 | 0.259 | 0.079 | 2.486E−15 | PCCB | 3.87E−14 | 0.777 | 0.239 | 0.048 | 3.776E−10 |
| RABAC1 | 2.62E−19 | −0.706 | 0.225 | 0.092 | 2.557E−15 | PSME3 | 4.12E−14 | 0.399 | 0.29 | 0.092 | 4.010E−10 |
| GBP1 | 3.13E−19 | −0.269 | 0.286 | 0.123 | 3.048E−15 | PPIP5K2 | 4.48E−14 | 0.754 | 0.271 | 0.057 | 4.364E−10 |
| ARPC4 | 3.25E−19 | −0.285 | 0.292 | 0.101 | 3.164E−15 | SNHG7 | 4.57E−14 | 0.301 | 0.212 | 0.035 | 4.452E−10 |
| ZNF593 | 3.31E−19 | −0.572 | 0.245 | 0.083 | 3.222E−15 | STX6 | 5.02E−14 | 0.297 | 0.244 | 0.061 | 4.888E−10 |
| IL10RB | 3.59E−19 | −0.434 | 0.272 | 0.101 | 3.499E−15 | ALG2 | 5.08E−14 | 0.483 | 0.269 | 0.079 | 4.955E−10 |
| RTCB | 3.76E−19 | −0.645 | 0.212 | 0.075 | 3.667E−15 | SYCP2 | 5.30E−14 | 0.509 | 0.256 | 0.061 | 5.170E−10 |
| EPHX1 | 4.23E−19 | −1.137 | 0.15 | 0.088 | 4.121E−15 | HIST1H4C | 5.62E−14 | 0.725 | 0.632 | 0.557 | 5.481E−10 |
| NPM3 | 5.70E−19 | −1.083 | 0.209 | 0.123 | 5.555E−15 | UHRF1BP1L | 6.44E−14 | 0.389 | 0.179 | 0.035 | 6.278E−10 |
| NAPA | 5.94E−19 | −0.432 | 0.25 | 0.105 | 5.793E−15 | WASL | 6.58E−14 | 0.527 | 0.283 | 0.079 | 6.414E−10 |
| PRKCSH | 6.35E−19 | −0.359 | 0.265 | 0.083 | 6.192E−15 | PIGC | 7.22E−14 | 0.419 | 0.265 | 0.075 | 7.035E−10 |
| YY1AP1 | 6.42E−19 | −0.726 | 0.225 | 0.118 | 6.254E−15 | TMX3 | 7.33E−14 | 0.397 | 0.277 | 0.101 | 7.144E−10 |
| MAP7 | 6.42E−19 | −0.460 | 0.301 | 0.114 | 6.261E−15 | MBD4 | 7.49E−14 | 0.462 | 0.234 | 0.053 | 7.298E−10 |
| C1orf131 | 6.76E−19 | −0.498 | 0.334 | 0.158 | 6.589E−15 | PLA2G4A | 7.59E−14 | 0.954 | 0.265 | 0.053 | 7.394E−10 |
| ZNF33A | 6.93E−19 | −0.256 | 0.309 | 0.114 | 6.749E−15 | AGGF1 | 7.70E−14 | 0.420 | 0.204 | 0.031 | 7.502E−10 |
| C17orf62 | 7.46E−19 | −0.339 | 0.248 | 0.066 | 7.267E−15 | RPL11 | 7.85E−14 | 0.361 | 0.97 | 0.825 | 7.648E−10 |
| TMED3 | 9.67E−19 | −0.432 | 0.244 | 0.092 | 9.422E−15 | TBC1D1 | 8.05E−14 | 1.267 | 0.259 | 0.053 | 7.840E−10 |
| RABIF | 1.02E−18 | −0.509 | 0.3 | 0.105 | 9.987E−15 | NRBP1 | 8.52E−14 | 0.752 | 0.248 | 0.075 | 8.300E−10 |
| DGCR6L | 1.12E−18 | −0.697 | 0.174 | 0.048 | 1.096E−14 | MYNN | 8.55E−14 | 0.587 | 0.297 | 0.101 | 8.333E−10 |
| IDH1 | 1.24E−18 | −0.286 | 0.287 | 0.07 | 1.205E−14 | SENP2 | 8.81E−14 | 0.524 | 0.245 | 0.057 | 8.586E−10 |
| KIAA1143 | 1.37E−18 | −0.578 | 0.177 | 0.044 | 1.332E−14 | ZNF518A | 9.08E−14 | 0.904 | 0.227 | 0.044 | 8.853E−10 |
| RP11- | 1.68E−18 | −0.436 | 0.147 | 0.031 | 1.633E−14 | NCALD | 9.33E−14 | 0.491 | 0.189 | 0.048 | 9.093E−10 |
| 108M9.4 | | | | | | | | | | | |
| FRMD4A | 1.82E−18 | −0.374 | 0.25 | 0.092 | 1.778E−14 | HAUS2 | 9.71E−14 | 0.326 | 0.253 | 0.057 | 9.465E−10 |
| GDI1 | 1.84E−18 | −0.520 | 0.21 | 0.07 | 1.791E−14 | AIFM1 | 9.96E−14 | 0.288 | 0.269 | 0.079 | 9.704E−10 |
| CNPY3 | 1.90E−18 | −0.352 | 0.23 | 0.07 | 1.854E−14 | RALGPS2 | 1.06E−13 | 0.610 | 0.271 | 0.075 | 1.030E−09 |
| MBOAT1 | 1.95E−18 | −0.718 | 0.218 | 0.083 | 1.904E−14 | MSH6 | 1.06E−13 | 0.310 | 0.301 | 0.096 | 1.033E−09 |
| MTSS1L | 2.05E−18 | −0.986 | 0.116 | 0.053 | 1.999E−14 | C12orf45 | 1.06E−13 | 0.599 | 0.244 | 0.048 | 1.036E−09 |
| XPNPEP1 | 2.24E−18 | −1.034 | 0.174 | 0.079 | 2.183E−14 | RAB3IP | 1.32E−13 | 0.455 | 0.227 | 0.048 | 1.291E−09 |
| IMPDH2 | 2.46E−18 | −0.440 | 0.245 | 0.096 | 2.398E−14 | EPC1 | 1.33E−13 | 0.312 | 0.268 | 0.083 | 1.300E−09 |
| SENP5 | 2.52E−18 | −0.262 | 0.309 | 0.123 | 2.457E−14 | C11orf74 | 1.35E−13 | 0.743 | 0.213 | 0.026 | 1.317E−09 |
| GOLM1 | 3.09E−18 | −0.300 | 0.298 | 0.123 | 3.013E−14 | ECI1 | 1.45E−13 | 0.582 | 0.21 | 0.031 | 1.411E−09 |
| HMOX2 | 3.22E−18 | −0.280 | 0.284 | 0.101 | 3.137E−14 | TMED7 | 1.49E−13 | 0.698 | 0.21 | 0.035 | 1.450E−09 |
| IAH1 | 3.38E−18 | −0.772 | 0.204 | 0.07 | 3.289E−14 | UEVLD | 1.53E−13 | 0.642 | 0.23 | 0.039 | 1.492E−09 |
| YKT6 | 3.39E−18 | −0.325 | 0.295 | 0.14 | 3.308E−14 | HLTF | 1.55E−13 | 0.265 | 0.271 | 0.083 | 1.514E−09 |
| SLC29A1 | 3.86E−18 | −0.581 | 0.253 | 0.092 | 3.759E−14 | UBTF | 1.57E−13 | 0.635 | 0.254 | 0.079 | 1.526E−09 |
| DTX3L | 4.00E−18 | −0.345 | 0.312 | 0.123 | 3.894E−14 | REXO2 | 1.66E−13 | 0.963 | 0.23 | 0.035 | 1.621E−09 |
| RHOQ | 4.03E−18 | −1.210 | 0.172 | 0.088 | 3.925E−14 | FZD6 | 1.73E−13 | 0.639 | 0.242 | 0.048 | 1.684E−09 |
| G6PD | 4.06E−18 | −0.960 | 0.165 | 0.061 | 3.961E−14 | BMPR2 | 1.73E−13 | 0.479 | 0.239 | 0.057 | 1.686E−09 |
| HLA-DPB1 | 4.08E−18 | −1.237 | 0.13 | 0.044 | 3.979E−14 | UBE2H | 1.75E−13 | 0.473 | 0.278 | 0.079 | 1.707E−09 |
| PPP6C | 4.14E−18 | −0.565 | 0.224 | 0.083 | 4.036E−14 | ASAP1 | 1.87E−13 | 0.720 | 0.191 | 0.031 | 1.821E−09 |
| URM1 | 4.15E−18 | −0.314 | 0.233 | 0.092 | 4.047E−14 | ATAD2 | 2.00E−13 | 0.301 | 0.345 | 0.154 | 1.952E−09 |
| TRAF7 | 4.89E−18 | −0.635 | 0.227 | 0.092 | 4.760E−14 | TEX30 | 2.02E−13 | 0.525 | 0.31 | 0.123 | 1.970E−09 |
| SSR4 | 4.91E−18 | −0.297 | 0.936 | 0.807 | 4.786E−14 | IVD | 2.07E−13 | 1.063 | 0.245 | 0.057 | 2.021E−09 |
| MAX | 5.26E−18 | −0.487 | 0.268 | 0.092 | 5.126E−14 | USP18 | 2.15E−13 | 0.597 | 0.209 | 0.039 | 2.096E−09 |
| ARID2 | 5.37E−18 | −0.625 | 0.245 | 0.118 | 5.236E−14 | HPS5 | 2.23E−13 | 0.578 | 0.231 | 0.053 | 2.168E−09 |
| MAP1B | 5.38E−18 | −0.296 | 0.368 | 0.175 | 5.241E−14 | ATR | 2.33E−13 | 0.439 | 0.312 | 0.118 | 2.270E−09 |
| ANK3 | 5.84E−18 | −0.336 | 0.33 | 0.132 | 5.692E−14 | PTS | 2.33E−13 | 0.337 | 0.266 | 0.079 | 2.271E−09 |
| SAMM50 | 6.11E−18 | −0.355 | 0.231 | 0.079 | 5.954E−14 | MED30 | 2.36E−13 | 0.588 | 0.307 | 0.088 | 2.298E−09 |
| CUL4A | 6.29E−18 | −0.391 | 0.303 | 0.145 | 6.132E−14 | HUS1 | 2.39E−13 | 0.251 | 0.228 | 0.07 | 2.329E−09 |
| MRPL28 | 6.42E−18 | −0.714 | 0.275 | 0.162 | 6.260E−14 | TAF1D | 2.43E−13 | 0.456 | 0.242 | 0.07 | 2.367E−09 |
| CSNK1D | 6.48E−18 | −0.267 | 0.272 | 0.123 | 6.315E−14 | EMC3 | 2.50E−13 | 0.546 | 0.293 | 0.101 | 2.434E−09 |
| CCDC34 | 6.63E−18 | −0.499 | 0.309 | 0.14 | 6.462E−14 | RRAGA | 2.52E−13 | 0.332 | 0.174 | 0.035 | 2.454E−09 |
| FRG1 | 7.35E−18 | −0.955 | 0.175 | 0.07 | 7.160E−14 | SAE1 | 2.56E−13 | 0.357 | 0.224 | 0.057 | 2.491E−09 |
| KPNA6 | 7.42E−18 | −0.279 | 0.257 | 0.079 | 7.235E−14 | ABI2 | 2.74E−13 | 0.564 | 0.225 | 0.044 | 2.673E−09 |
| FAM104B | 8.41E−18 | −0.477 | 0.236 | 0.083 | 8.196E−14 | MPP5 | 3.24E−13 | 0.535 | 0.275 | 0.079 | 3.161E−09 |
| FLOT1 | 8.77E−18 | −0.356 | 0.271 | 0.096 | 8.543E−14 | ALG13 | 3.40E−13 | 0.827 | 0.197 | 0.022 | 3.314E−09 |
| WDR1 | 8.93E−18 | −0.592 | 0.284 | 0.132 | 8.700E−14 | 7-Mar | 3.63E−13 | 0.632 | 0.304 | 0.092 | 3.533E−09 |
| RNASET2 | 9.65E−18 | −0.320 | 0.221 | 0.044 | 9.404E−14 | SNX4 | 3.72E−13 | 0.488 | 0.242 | 0.057 | 3.621E−09 |
| NDUFA9 | 1.03E−17 | −0.586 | 0.239 | 0.096 | 1.006E−13 | NDFIP2 | 4.21E−13 | 0.319 | 0.231 | 0.066 | 4.099E−09 |
| BRD4 | 1.06E−17 | −0.373 | 0.337 | 0.18 | 1.029E−13 | KANK1 | 4.27E−13 | 0.297 | 0.251 | 0.092 | 4.159E−09 |
| C9orf89 | 1.07E−17 | −0.315 | 0.253 | 0.105 | 1.041E−13 | DLG1 | 4.30E−13 | 0.398 | 0.31 | 0.127 | 4.189E−09 |
| MBOAT7 | 1.07E−17 | −0.863 | 0.153 | 0.057 | 1.047E−13 | UBL4A | 4.63E−13 | 0.477 | 0.209 | 0.039 | 4.508E−09 |
| CHTOP | 1.11E−17 | −0.566 | 0.236 | 0.092 | 1.082E−13 | GUF1 | 4.86E−13 | 0.285 | 0.289 | 0.105 | 4.731E−09 |
| CDC42EP4 | 1.12E−17 | −0.436 | 0.23 | 0.079 | 1.093E−13 | ZFAND2A | 4.95E−13 | 0.489 | 0.293 | 0.11 | 4.828E−09 |
| MAPK13 | 1.17E−17 | −0.801 | 0.209 | 0.118 | 1.142E−13 | STAM2 | 4.96E−13 | 0.930 | 0.233 | 0.048 | 4.837E−09 |
| NUBP1 | 1.20E−17 | −0.584 | 0.185 | 0.061 | 1.169E−13 | CREB3 | 4.97E−13 | 0.661 | 0.25 | 0.057 | 4.841E−09 |
| UBE2G2 | 1.34E−17 | −0.329 | 0.262 | 0.092 | 1.303E−13 | MYO10 | 5.27E−13 | 0.622 | 0.23 | 0.053 | 5.134E−09 |
| TMSB15B | 1.72E−17 | −0.868 | 0.139 | 0.048 | 1.679E−13 | USP22 | 5.40E−13 | 0.266 | 0.238 | 0.057 | 5.263E−09 |
| TMEM54 | 1.85E−17 | −0.655 | 0.163 | 0.035 | 1.801E−13 | RNF8 | 6.27E−13 | 0.984 | 0.259 | 0.053 | 6.113E−09 |
| NMRAL1 | 1.94E−17 | −0.549 | 0.2 | 0.057 | 1.893E−13 | RAB11FIP1 | 6.46E−13 | 0.569 | 0.271 | 0.075 | 6.294E−09 |
| ARL16 | 1.98E−17 | −0.264 | 0.265 | 0.088 | 1.931E−13 | STAM | 6.56E−13 | 0.605 | 0.204 | 0.039 | 6.396E−09 |
| LAMTOR1 | 2.05E−17 | −0.444 | 0.254 | 0.096 | 1.996E−13 | RPRD1A | 6.72E−13 | 0.299 | 0.266 | 0.083 | 6.545E−09 |
| C9orf142 | 2.08E−17 | −0.474 | 0.216 | 0.07 | 2.027E−13 | FUCA2 | 6.88E−13 | 0.382 | 0.277 | 0.092 | 6.703E−09 |
| CAPS | 2.15E−17 | −0.530 | 0.163 | 0.044 | 2.098E−13 | SH3BGR | 6.89E−13 | 0.507 | 0.2 | 0.048 | 6.710E−09 |
| FKBP5 | 2.27E−17 | −0.708 | 0.278 | 0.136 | 2.209E−13 | USP38 | 6.96E−13 | 0.428 | 0.189 | 0.039 | 6.786E−09 |
| RPS14 | 2.29E−17 | −0.400 | 0.961 | 0.882 | 2.228E−13 | NRP2 | 6.98E−13 | 0.576 | 0.228 | 0.057 | 6.805E−09 |
| NSRP1 | 2.48E−17 | −0.514 | 0.256 | 0.088 | 2.413E−13 | SLC30A5 | 7.14E−13 | 0.666 | 0.183 | 0.031 | 6.957E−09 |
| PABPN1 | 2.66E−17 | −0.532 | 0.182 | 0.044 | 2.591E−13 | DYNC1LI1 | 7.82E−13 | 0.349 | 0.272 | 0.092 | 7.624E−09 |
| PDCD6IP | 2.73E−17 | −0.303 | 0.277 | 0.096 | 2.662E−13 | ING1 | 7.93E−13 | 1.089 | 0.182 | 0.013 | 7.729E−09 |
| SDHA | 2.90E−17 | −0.651 | 0.195 | 0.079 | 2.829E−13 | GINS2 | 8.00E−13 | 0.309 | 0.23 | 0.061 | 7.791E−09 |
| CHD8 | 3.02E−17 | −0.276 | 0.352 | 0.162 | 2.944E−13 | RPA2 | 8.26E−13 | 0.326 | 0.183 | 0.026 | 8.049E−09 |
| ZNF480 | 3.09E−17 | −0.326 | 0.222 | 0.061 | 3.012E−13 | VAMP3 | 8.91E−13 | 0.721 | 0.239 | 0.057 | 8.683E−09 |
| ANXA3 | 3.26E−17 | −0.323 | 0.39 | 0.224 | 3.179E−13 | LTN1 | 9.03E−13 | 0.433 | 0.247 | 0.075 | 8.802E−09 |
| NUBP2 | 3.50E−17 | −0.864 | 0.209 | 0.092 | 3.410E−13 | WDR3 | 9.14E−13 | 0.346 | 0.293 | 0.11 | 8.907E−09 |
| FLNB | 3.56E−17 | −0.721 | 0.239 | 0.127 | 3.471E−13 | DIS3 | 9.61E−13 | 0.322 | 0.257 | 0.092 | 9.361E−09 |
| DPP7 | 3.73E−17 | −1.024 | 0.165 | 0.075 | 3.638E−13 | GOPC | 9.69E−13 | 0.758 | 0.238 | 0.057 | 9.446E−09 |
| SHARPIN | 3.82E−17 | −0.722 | 0.163 | 0.048 | 3.726E−13 | PLCG2 | 9.86E−13 | 0.624 | 0.204 | 0.035 | 9.605E−09 |
| IFI27L1 | 3.85E−17 | −1.360 | 0.118 | 0.096 | 3.756E−13 | CCDC117 | 1.06E−12 | 0.838 | 0.284 | 0.079 | 1.029E−08 |
| TYMS | 3.92E−17 | −0.453 | 0.201 | 0.057 | 3.824E−13 | TF | 1.06E−12 | 0.669 | 0.253 | 0.127 | 1.036E−08 |
| RUVBL2 | 3.98E−17 | −0.409 | 0.216 | 0.083 | 3.879E−13 | CCDC43 | 1.07E−12 | 0.291 | 0.227 | 0.07 | 1.039E−08 |
| TOMM40 | 4.18E−17 | −0.476 | 0.163 | 0.035 | 4.076E−13 | PSMD5 | 1.12E−12 | 0.597 | 0.218 | 0.048 | 1.091E−08 |
| RAB40B | 4.56E−17 | −0.264 | 0.256 | 0.083 | 4.448E−13 | HERC5 | 1.22E−12 | 0.395 | 0.216 | 0.092 | 1.189E−08 |
| FKBP8 | 4.63E−17 | −0.518 | 0.153 | 0.044 | 4.509E−13 | USP36 | 1.23E−12 | 0.316 | 0.239 | 0.061 | 1.197E−08 |
| C11orf83 | 5.23E−17 | −0.700 | 0.2 | 0.092 | 5.101E−13 | LRRC1 | 1.28E−12 | 0.437 | 0.212 | 0.057 | 1.250E−08 |
| ACO2 | 6.21E−17 | −0.477 | 0.248 | 0.092 | 6.048E−13 | TMEM223 | 1.35E−12 | 0.303 | 0.234 | 0.088 | 1.314E−08 |
| SAMD9L | 6.29E−17 | −0.570 | 0.2 | 0.096 | 6.128E−13 | PRLR | 1.40E−12 | 0.408 | 0.272 | 0.092 | 1.361E−08 |
| INO80B | 6.48E−17 | −0.656 | 0.156 | 0.048 | 6.312E−13 | ANXA4 | 1.41E−12 | 0.255 | 0.254 | 0.083 | 1.379E−08 |
| PRRC2A | 6.53E−17 | −1.124 | 0.12 | 0.061 | 6.365E−13 | MLF1 | 1.42E−12 | 0.296 | 0.254 | 0.083 | 1.385E−08 |
| FBLIM1 | 6.65E−17 | −0.354 | 0.198 | 0.039 | 6.483E−13 | CCNG1 | 1.42E−12 | 0.703 | 0.265 | 0.079 | 1.387E−08 |
| SNX17 | 6.72E−17 | −0.402 | 0.203 | 0.061 | 6.548E−13 | UCHL1 | 1.46E−12 | 0.361 | 0.218 | 0.057 | 1.428E−08 |
| MAP2 | 7.12E−17 | −0.893 | 0.251 | 0.136 | 6.943E−13 | PHYH | 1.52E−12 | 0.316 | 0.201 | 0.048 | 1.483E−08 |
| H1FX | 7.26E−17 | −0.405 | 0.165 | 0.026 | 7.074E−13 | RAD51B | 1.54E−12 | 0.706 | 0.192 | 0.026 | 1.499E−08 |
| DNAJC1 | 7.37E−17 | −0.387 | 0.333 | 0.145 | 7.179E−13 | ID3 | 1.64E−12 | 0.505 | 0.21 | 0.039 | 1.599E−08 |
| OGFOD3 | 7.96E−17 | −0.462 | 0.224 | 0.07 | 7.753E−13 | ACOX1 | 1.66E−12 | 0.658 | 0.222 | 0.044 | 1.619E−08 |
| C11orf48 | 8.51E−17 | −0.683 | 0.198 | 0.079 | 8.292E−13 | ARMC1 | 1.69E−12 | 0.806 | 0.231 | 0.048 | 1.643E−08 |
| BAD | 8.91E−17 | −0.781 | 0.197 | 0.075 | 8.680E−13 | MGST2 | 1.70E−12 | 0.496 | 0.185 | 0.031 | 1.653E−08 |
| FANCF | 1.03E−16 | −0.551 | 0.206 | 0.079 | 1.002E−12 | FNDC3A | 1.76E−12 | 0.332 | 0.198 | 0.048 | 1.712E−08 |
| SELM | 1.07E−16 | −0.642 | 0.169 | 0.079 | 1.038E−12 | HIST1H2AC | 1.87E−12 | 0.341 | 0.327 | 0.145 | 1.818E−08 |
| EIF4EBP1 | 1.08E−16 | −0.970 | 0.147 | 0.075 | 1.054E−12 | PRIM1 | 2.00E−12 | 0.392 | 0.197 | 0.053 | 1.946E−08 |
| TRIM47 | 1.11E−16 | −0.948 | 0.159 | 0.075 | 1.078E−12 | UMPS | 2.02E−12 | 0.319 | 0.182 | 0.048 | 1.970E−08 |
| PLXNB2 | 1.15E−16 | −0.379 | 0.244 | 0.096 | 1.120E−12 | NKTR | 2.11E−12 | 0.349 | 0.278 | 0.096 | 2.054E−08 |
| NUS1 | 1.35E−16 | −0.398 | 0.194 | 0.066 | 1.316E−12 | SMAD5 | 2.43E−12 | 0.375 | 0.221 | 0.061 | 2.364E−08 |
| IFI30 | 1.35E−16 | −1.586 | 0.085 | 0.123 | 1.319E−12 | RCHY1 | 2.49E−12 | 0.563 | 0.262 | 0.07 | 2.429E−08 |
| FOXK2 | 1.52E−16 | −0.259 | 0.248 | 0.096 | 1.484E−12 | NUP133 | 2.55E−12 | 0.307 | 0.186 | 0.044 | 2.490E−08 |
| EMG1 | 1.72E−16 | −0.395 | 0.194 | 0.066 | 1.674E−12 | MYCBP2 | 2.58E−12 | 0.284 | 0.224 | 0.057 | 2.513E−08 |
| FKBP2 | 1.74E−16 | −0.677 | 0.239 | 0.096 | 1.698E−12 | BFAR | 2.59E−12 | 0.468 | 0.189 | 0.026 | 2.528E−08 |
| USF2 | 1.82E−16 | −0.594 | 0.183 | 0.066 | 1.773E−12 | UGDH | 2.73E−12 | 0.294 | 0.233 | 0.066 | 2.656E−08 |
| TIMP1 | 1.98E−16 | −0.739 | 0.191 | 0.101 | 1.931E−12 | KIAA1841 | 2.73E−12 | 0.791 | 0.265 | 0.07 | 2.657E−08 |
| DPYSL2 | 2.00E−16 | −0.493 | 0.213 | 0.066 | 1.949E−12 | PSMD10 | 2.84E−12 | 0.475 | 0.275 | 0.088 | 2.770E−08 |
| ZC3HAV1 | 2.01E−16 | −0.265 | 0.263 | 0.105 | 1.954E−12 | ORMDL1 | 2.87E−12 | 0.355 | 0.259 | 0.075 | 2.793E−08 |
| RSBN1L | 2.09E−16 | −0.577 | 0.215 | 0.079 | 2.038E−12 | HSPA13 | 2.92E−12 | 0.724 | 0.25 | 0.07 | 2.848E−08 |
| UBE2V2 | 2.11E−16 | −0.388 | 0.322 | 0.154 | 2.054E−12 | GLIPR1 | 2.96E−12 | 0.479 | 0.203 | 0.044 | 2.881E−08 |
| ZBED1 | 2.22E−16 | −0.350 | 0.209 | 0.066 | 2.161E−12 | RP11- | 2.99E−12 | 0.327 | 0.139 | 0.018 | 2.918E−08 |
| | | | | | | 841O20.2 | | | | | |
| ACBD7 | 2.32E−16 | −0.529 | 0.165 | 0.048 | 2.262E−12 | SPAST | 3.09E−12 | 0.262 | 0.192 | 0.039 | 3.010E−08 |
| PLEKHB1 | 2.39E−16 | −0.365 | 0.225 | 0.083 | 2.333E−12 | GALNT7 | 3.34E−12 | 1.041 | 0.163 | 0.018 | 3.255E−08 |
| CYSTM1 | 2.43E−16 | −0.429 | 0.245 | 0.105 | 2.372E−12 | THUMPD3 | 3.36E−12 | 0.456 | 0.221 | 0.066 | 3.274E−08 |
| CDS1 | 2.48E−16 | −0.529 | 0.287 | 0.175 | 2.419E−12 | TRIM26 | 3.49E−12 | 0.322 | 0.209 | 0.048 | 3.403E−08 |
| TRIM28 | 2.55E−16 | −0.919 | 0.127 | 0.048 | 2.481E−12 | SCFD1 | 3.87E−12 | 0.482 | 0.219 | 0.053 | 3.775E−08 |
| THOC3 | 2.81E−16 | −0.320 | 0.169 | 0.048 | 2.740E−12 | PHKB | 4.25E−12 | 0.284 | 0.234 | 0.075 | 4.145E−08 |
| PHAX | 2.85E−16 | −0.347 | 0.242 | 0.075 | 2.775E−12 | KDM1A | 4.36E−12 | 0.337 | 0.209 | 0.044 | 4.252E−08 |
| RNF114 | 2.86E−16 | −0.404 | 0.306 | 0.145 | 2.791E−12 | MTIF2 | 4.61E−12 | 0.411 | 0.286 | 0.101 | 4.490E−08 |
| IST1 | 3.00E−16 | −0.420 | 0.247 | 0.105 | 2.919E−12 | ARHGAP10 | 4.79E−12 | 0.732 | 0.157 | 0.031 | 4.670E−08 |
| TLK2 | 3.07E−16 | −0.484 | 0.206 | 0.07 | 2.991E−12 | PCYT1A | 4.99E−12 | 0.569 | 0.197 | 0.039 | 4.865E−08 |
| NINJ1 | 3.22E−16 | −0.956 | 0.168 | 0.092 | 3.136E−12 | C12orf44 | 5.17E−12 | 0.269 | 0.177 | 0.039 | 5.042E−08 |
| SURF6 | 3.31E−16 | −0.571 | 0.21 | 0.105 | 3.225E−12 | SGMS1 | 5.30E−12 | 0.302 | 0.179 | 0.035 | 5.166E−08 |
| TVP23B | 3.75E−16 | −0.470 | 0.115 | 0.031 | 3.656E−12 | ZDHHC3 | 5.45E−12 | 0.878 | 0.185 | 0.022 | 5.308E−08 |
| PUM2 | 3.82E−16 | −0.350 | 0.31 | 0.184 | 3.727E−12 | DBR1 | 5.55E−12 | 0.573 | 0.2 | 0.031 | 5.405E−08 |
| DDX42 | 3.88E−16 | −0.264 | 0.321 | 0.127 | 3.780E−12 | IQCE | 5.68E−12 | 0.751 | 0.162 | 0.022 | 5.535E−08 |
| RP11- | 4.53E−16 | −0.346 | 0.192 | 0.039 | 4.418E−12 | FOPNL | 5.86E−12 | 0.281 | 0.248 | 0.07 | 5.708E−08 |
| 357C3.3 | | | | | | | | | | | |
| PLD3 | 5.16E−16 | −1.080 | 0.151 | 0.07 | 5.031E−12 | DGKH | 5.90E−12 | 0.336 | 0.212 | 0.057 | 5.752E−08 |
| KPNA1 | 5.84E−16 | −0.285 | 0.278 | 0.114 | 5.689E−12 | SRA1 | 5.95E−12 | 0.393 | 0.172 | 0.083 | 5.794E−08 |
| ASNA1 | 5.98E−16 | −0.431 | 0.188 | 0.079 | 5.832E−12 | UBR7 | 6.35E−12 | 0.392 | 0.153 | 0.044 | 6.192E−08 |
| UBE2V1 | 6.38E−16 | −1.044 | 0.113 | 0.061 | 6.219E−12 | SUV39H2 | 6.47E−12 | 0.382 | 0.26 | 0.079 | 6.305E−08 |
| SRCAP | 6.55E−16 | −0.822 | 0.188 | 0.083 | 6.386E−12 | MYSM1 | 6.50E−12 | 0.533 | 0.204 | 0.039 | 6.335E−08 |
| PMM1 | 6.56E−16 | −0.387 | 0.182 | 0.053 | 6.389E−12 | NQO1 | 6.73E−12 | 0.444 | 0.216 | 0.048 | 6.554E−08 |
| FLAD1 | 6.80E−16 | −0.587 | 0.218 | 0.075 | 6.626E−12 | SLC15A3 | 6.91E−12 | 0.266 | 0.16 | 0.022 | 6.738E−08 |
| FAM96B | 7.02E−16 | −0.273 | 0.266 | 0.123 | 6.844E−12 | FKBP14 | 7.35E−12 | 0.441 | 0.188 | 0.039 | 7.164E−08 |
| CTSZ | 7.53E−16 | −0.584 | 0.212 | 0.092 | 7.336E−12 | ARL5A | 7.39E−12 | 0.773 | 0.239 | 0.066 | 7.202E−08 |
| NOTCH2 | 7.90E−16 | −0.449 | 0.234 | 0.101 | 7.700E−12 | CHML | 7.42E−12 | 0.586 | 0.225 | 0.053 | 7.231E−08 |
| C7orf55- | 8.27E−16 | −0.891 | 0.135 | 0.061 | 8.062E−12 | HK1 | 7.42E−12 | 0.636 | 0.191 | 0.039 | 7.232E−08 |
| LUC7L2 | | | | | | | | | | | |
| SLK | 8.45E−16 | −0.278 | 0.292 | 0.127 | 8.234E−12 | C15orf38 | 7.52E−12 | 0.456 | 0.141 | 0.013 | 7.327E−08 |
| DLG3 | 8.51E−16 | −0.428 | 0.278 | 0.127 | 8.288E−12 | C14orf142 | 7.64E−12 | 0.416 | 0.212 | 0.061 | 7.446E−08 |
| GABARAP | 8.66E−16 | −0.865 | 0.106 | 0.035 | 8.440E−12 | ARID5B | 7.92E−12 | 0.410 | 0.265 | 0.092 | 7.722E−08 |
| RP11- | 8.72E−16 | −0.977 | 0.191 | 0.127 | 8.498E−12 | DHX32 | 8.02E−12 | 0.680 | 0.194 | 0.035 | 7.817E−08 |
| 485G4.2 | | | | | | | | | | | |
| GIMAP7 | 9.00E−16 | −2.571 | 0.009 | 0.114 | 8.774E−12 | TTC13 | 8.32E−12 | 0.283 | 0.177 | 0.053 | 8.112E−08 |
| KDM2A | 9.74E−16 | −0.455 | 0.207 | 0.07 | 9.489E−12 | ARFGAP2 | 8.65E−12 | 0.380 | 0.2 | 0.048 | 8.427E−08 |
| GLYR1 | 1.04E−15 | −0.267 | 0.304 | 0.14 | 1.016E−11 | PHACTR4 | 8.77E−12 | 0.499 | 0.272 | 0.096 | 8.549E−08 |
| SEPHS1 | 1.05E−15 | −0.471 | 0.203 | 0.07 | 1.026E−11 | CSNK1G3 | 8.94E−12 | 0.317 | 0.204 | 0.053 | 8.710E−08 |
| SLC39A4 | 1.13E−15 | −0.603 | 0.195 | 0.075 | 1.104E−11 | TRIM27 | 9.63E−12 | 0.772 | 0.203 | 0.039 | 9.381E−08 |
| TIMM8B | 1.14E−15 | −0.520 | 0.18 | 0.061 | 1.107E−11 | RASGEF1B | 1.04E−11 | 0.610 | 0.221 | 0.053 | 1.011E−07 |
| PIN1 | 1.27E−15 | −0.320 | 0.25 | 0.096 | 1.233E−11 | RCAN3 | 1.06E−11 | 0.450 | 0.269 | 0.088 | 1.037E−07 |
| PIH1D1 | 1.29E−15 | −0.884 | 0.157 | 0.083 | 1.257E−11 | C21orf33 | 1.09E−11 | 1.373 | 0.204 | 0.031 | 1.064E−07 |
| MAPRE2 | 1.54E−15 | −0.262 | 0.277 | 0.114 | 1.498E−11 | PRMT3 | 1.11E−11 | 0.429 | 0.236 | 0.07 | 1.077E−07 |
| RHOT2 | 1.64E−15 | −0.377 | 0.168 | 0.044 | 1.602E−11 | CEP57L1 | 1.11E−11 | 0.366 | 0.171 | 0.039 | 1.085E−07 |
| BRI3 | 1.72E−15 | −0.889 | 0.145 | 0.07 | 1.681E−11 | PRDM2 | 1.12E−11 | 0.432 | 0.231 | 0.066 | 1.095E−07 |
| CRIM1 | 1.80E−15 | −0.474 | 0.244 | 0.101 | 1.753E−11 | DICER1 | 1.19E−11 | 0.340 | 0.268 | 0.096 | 1.161E−07 |
| SLC35A2 | 1.81E−15 | −0.577 | 0.2 | 0.079 | 1.765E−11 | SP1 | 1.20E−11 | 0.529 | 0.162 | 0.018 | 1.172E−07 |
| LSM10 | 2.02E−15 | −0.762 | 0.177 | 0.079 | 1.966E−11 | ZCCHC17 | 1.38E−11 | 0.430 | 0.263 | 0.088 | 1.341E−07 |
| TWF2 | 2.02E−15 | −0.275 | 0.175 | 0.039 | 1.969E−11 | CARD6 | 1.48E−11 | 1.106 | 0.191 | 0.031 | 1.443E−07 |
| ILVBL | 2.13E−15 | −0.418 | 0.215 | 0.083 | 2.071E−11 | PARP2 | 1.50E−11 | 0.498 | 0.174 | 0.026 | 1.457E−07 |
| MT-RNR1 | 2.22E−15 | −0.316 | 0.989 | 0.947 | 2.163E−11 | R3HDM1 | 1.58E−11 | 0.526 | 0.183 | 0.061 | 1.537E−07 |
| SLC27A5 | 2.23E−15 | −0.838 | 0.169 | 0.079 | 2.175E−11 | CDK5RAP2 | 1.67E−11 | 0.487 | 0.286 | 0.096 | 1.625E−07 |
| ZYX | 2.38E−15 | −0.776 | 0.127 | 0.053 | 2.322E−11 | ELL2 | 1.78E−11 | 0.684 | 0.18 | 0.031 | 1.733E−07 |
| SORBS2 | 2.41E−15 | −0.270 | 0.272 | 0.118 | 2.348E−11 | UNC50 | 1.83E−11 | 0.269 | 0.194 | 0.048 | 1.782E−07 |
| SHMT2 | 2.42E−15 | −0.579 | 0.165 | 0.057 | 2.362E−11 | P4HA1 | 1.93E−11 | 0.536 | 0.228 | 0.057 | 1.885E−07 |
| NNT | 2.45E−15 | −0.437 | 0.262 | 0.123 | 2.392E−11 | BLOC1S6 | 2.09E−11 | 0.252 | 0.233 | 0.079 | 2.033E−07 |
| BRI3BP | 2.67E−15 | −0.407 | 0.179 | 0.044 | 2.603E−11 | TRIM11 | 2.11E−11 | 0.327 | 0.127 | 0.013 | 2.054E−07 |
| TTYH3 | 2.72E−15 | −0.843 | 0.13 | 0.048 | 2.652E−11 | SKIV2L2 | 2.17E−11 | 0.772 | 0.238 | 0.075 | 2.117E−07 |
| SEPN1 | 3.31E−15 | −0.649 | 0.147 | 0.057 | 3.226E−11 | PLCH1 | 2.24E−11 | 0.401 | 0.233 | 0.075 | 2.184E−07 |
| ISG20 | 3.36E−15 | −0.635 | 0.186 | 0.083 | 3.272E−11 | MARK3 | 2.41E−11 | 0.449 | 0.228 | 0.061 | 2.346E−07 |
| MAD2L2 | 3.52E−15 | −0.983 | 0.177 | 0.092 | 3.430E−11 | WIBG | 2.44E−11 | 0.768 | 0.189 | 0.039 | 2.381E−07 |
| RHOG | 3.67E−15 | −0.646 | 0.192 | 0.079 | 3.574E−11 | DCAF10 | 2.75E−11 | 0.307 | 0.21 | 0.053 | 2.680E−07 |
| LMNB2 | 3.70E−15 | −0.362 | 0.2 | 0.053 | 3.609E−11 | FANCL | 2.77E−11 | 0.619 | 0.206 | 0.044 | 2.699E−07 |
| LMF2 | 3.75E−15 | −0.636 | 0.182 | 0.075 | 3.658E−11 | GNG12 | 2.79E−11 | 0.574 | 0.218 | 0.066 | 2.715E−07 |
| ATG4B | 3.81E−15 | −0.481 | 0.157 | 0.039 | 3.717E−11 | BCL2L13 | 2.84E−11 | 0.451 | 0.233 | 0.075 | 2.766E−07 |
| KIAA0319L | 3.99E−15 | −0.394 | 0.247 | 0.132 | 3.890E−11 | PRTFDC1 | 2.91E−11 | 0.528 | 0.201 | 0.048 | 2.836E−07 |
| POP7 | 4.00E−15 | −0.313 | 0.201 | 0.048 | 3.903E−11 | ZNF33B | 2.93E−11 | 0.483 | 0.186 | 0.031 | 2.856E−07 |
| RHOC | 4.01E−15 | −0.373 | 0.195 | 0.057 | 3.906E−11 | IMPACT | 2.94E−11 | 0.378 | 0.18 | 0.039 | 2.864E−07 |
| PGAM1 | 4.20E−15 | −1.010 | 0.104 | 0.039 | 4.094E−11 | NRIP1 | 2.94E−11 | 0.439 | 0.206 | 0.044 | 2.864E−07 |
| NOSIP | 4.25E−15 | −0.835 | 0.195 | 0.083 | 4.141E−11 | NES | 2.97E−11 | 0.319 | 0.156 | 0.026 | 2.895E−07 |
| TEX264 | 4.59E−15 | −0.694 | 0.175 | 0.083 | 4.468E−11 | AP1AR | 3.07E−11 | 0.373 | 0.222 | 0.057 | 2.995E−07 |
| SMCHD1 | 4.88E−15 | −0.454 | 0.244 | 0.101 | 4.759E−11 | PCDHB4 | 3.36E−11 | 0.392 | 0.139 | 0.022 | 3.273E−07 |
| RAP2B | 4.92E−15 | −0.458 | 0.245 | 0.118 | 4.796E−11 | AQR | 3.40E−11 | 0.608 | 0.182 | 0.031 | 3.317E−07 |
| BLVRB | 5.04E−15 | −0.337 | 0.212 | 0.079 | 4.913E−11 | BRD3 | 3.41E−11 | 0.334 | 0.248 | 0.088 | 3.326E−07 |
| ATN1 | 5.18E−15 | −0.346 | 0.219 | 0.075 | 5.052E−11 | MGEA5 | 3.43E−11 | 0.399 | 0.233 | 0.096 | 3.346E−07 |
| HIST1H2BC | 5.76E−15 | −0.370 | 0.292 | 0.162 | 5.611E−11 | BRWD1 | 3.49E−11 | 0.543 | 0.251 | 0.088 | 3.401E−07 |
| NBR1 | 6.12E−15 | −0.250 | 0.213 | 0.092 | 5.962E−11 | PAK1 | 3.66E−11 | 0.349 | 0.254 | 0.096 | 3.563E−07 |
| SLC38A10 | 6.62E−15 | −0.379 | 0.218 | 0.101 | 6.449E−11 | TERC | 3.83E−11 | 0.438 | 0.129 | 0.009 | 3.736E−07 |
| RERE | 6.72E−15 | −0.705 | 0.141 | 0.039 | 6.548E−11 | UTP6 | 3.94E−11 | 0.325 | 0.221 | 0.057 | 3.839E−07 |
| PSMD9 | 7.85E−15 | −1.122 | 0.116 | 0.057 | 7.649E−11 | DTNA | 4.08E−11 | 0.422 | 0.175 | 0.026 | 3.974E−07 |
| PEF1 | 7.99E−15 | −0.316 | 0.186 | 0.048 | 7.781E−11 | MCM3AP | 4.15E−11 | 0.383 | 0.166 | 0.022 | 4.048E−07 |
| SMAP1 | 8.17E−15 | −0.314 | 0.271 | 0.096 | 7.962E−11 | TMEM126A | 4.20E−11 | 0.403 | 0.185 | 0.031 | 4.096E−07 |
| FGGY | 8.34E−15 | −0.665 | 0.172 | 0.048 | 8.127E−11 | PNP | 4.67E−11 | 0.303 | 0.154 | 0.022 | 4.553E−07 |
| PDLIM1 | 8.64E−15 | −0.269 | 0.183 | 0.053 | 8.421E−11 | ZCCHC9 | 4.68E−11 | 0.510 | 0.186 | 0.031 | 4.560E−07 |
| AHNAK | 8.71E−15 | −0.338 | 0.357 | 0.219 | 8.489E−11 | USP5 | 4.70E−11 | 0.556 | 0.175 | 0.031 | 4.579E−07 |
| DCTN1 | 8.88E−15 | −0.333 | 0.222 | 0.083 | 8.651E−11 | GSTCD | 4.81E−11 | 0.807 | 0.133 | 0.009 | 4.689E−07 |
| MAGI1 | 9.02E−15 | −0.917 | 0.133 | 0.075 | 8.787E−11 | TRDC | 4.88E−11 | 0.299 | 0.147 | 0.031 | 4.760E−07 |
| GALK1 | 1.01E−14 | −0.778 | 0.162 | 0.079 | 9.833E−11 | KLHL36 | 4.97E−11 | 0.390 | 0.222 | 0.075 | 4.839E−07 |
| E2F3 | 1.06E−14 | −0.380 | 0.204 | 0.088 | 1.033E−10 | DIDO1 | 5.02E−11 | 0.520 | 0.228 | 0.07 | 4.891E−07 |
| CDK11A | 1.15E−14 | −0.492 | 0.142 | 0.048 | 1.121E−10 | DUSP1 | 5.04E−11 | 0.880 | 0.121 | 0.009 | 4.912E−07 |
| C2orf68 | 1.16E−14 | −0.899 | 0.136 | 0.079 | 1.126E−10 | DPH3 | 5.14E−11 | 0.270 | 0.236 | 0.092 | 5.007E−07 |
| FRG1B | 1.16E−14 | −0.563 | 0.177 | 0.061 | 1.132E−10 | SMIM19 | 5.21E−11 | 0.529 | 0.166 | 0.026 | 5.075E−07 |
| EIF2B4 | 1.26E−14 | −0.253 | 0.216 | 0.048 | 1.229E−10 | CPD | 5.42E−11 | 0.486 | 0.185 | 0.035 | 5.277E−07 |
| ANXA6 | 1.30E−14 | −0.253 | 0.228 | 0.096 | 1.264E−10 | SCAMP2 | 5.48E−11 | 0.578 | 0.165 | 0.035 | 5.336E−07 |
| RANGAP1 | 1.39E−14 | −0.371 | 0.175 | 0.044 | 1.354E−10 | PRKD3 | 5.65E−11 | 0.333 | 0.209 | 0.057 | 5.504E−07 |
| NUDT16L1 | 1.68E−14 | −0.386 | 0.177 | 0.053 | 1.634E−10 | ST3GAL1 | 6.31E−11 | 0.385 | 0.248 | 0.083 | 6.148E−07 |
| TRAPPC6A | 1.68E−14 | −0.596 | 0.126 | 0.035 | 1.639E−10 | ALG6 | 6.57E−11 | 0.599 | 0.183 | 0.039 | 6.400E−07 |
| BAX | 1.73E−14 | −0.777 | 0.154 | 0.066 | 1.684E−10 | CHRAC1 | 6.66E−11 | 0.305 | 0.248 | 0.083 | 6.493E−07 |
| GBP2 | 1.74E−14 | −0.621 | 0.195 | 0.088 | 1.699E−10 | ELK4 | 6.96E−11 | 0.288 | 0.241 | 0.096 | 6.782E−07 |
| WDR82 | 1.86E−14 | −0.870 | 0.162 | 0.083 | 1.812E−10 | LYSMD2 | 7.00E−11 | 0.287 | 0.153 | 0.018 | 6.817E−07 |
| DDX54 | 2.01E−14 | −0.256 | 0.231 | 0.066 | 1.963E−10 | RPRD2 | 7.38E−11 | 0.390 | 0.221 | 0.061 | 7.196E−07 |
| HEXA | 2.05E−14 | −0.533 | 0.204 | 0.088 | 1.997E−10 | VPS45 | 7.46E−11 | 0.282 | 0.144 | 0.026 | 7.272E−07 |
| SDCBP | 2.07E−14 | −0.901 | 0.209 | 0.105 | 2.022E−10 | CCP110 | 7.55E−11 | 0.323 | 0.175 | 0.044 | 7.357E−07 |
| APEH | 2.16E−14 | −0.806 | 0.144 | 0.061 | 2.105E−10 | NFX1 | 7.55E−11 | 0.356 | 0.195 | 0.039 | 7.362E−07 |
| UBALD2 | 2.18E−14 | −0.624 | 0.163 | 0.048 | 2.127E−10 | PPP2R2D | 7.60E−11 | 0.424 | 0.179 | 0.035 | 7.403E−07 |
| SRP19 | 2.22E−14 | −0.641 | 0.191 | 0.061 | 2.165E−10 | BRPF3 | 7.68E−11 | 1.234 | 0.169 | 0.018 | 7.480E−07 |
| IGSF8 | 2.23E−14 | −0.855 | 0.136 | 0.057 | 2.169E−10 | NUPL1 | 8.09E−11 | 0.299 | 0.26 | 0.118 | 7.886E−07 |
| PM20D2 | 2.30E−14 | −0.302 | 0.272 | 0.079 | 2.239E−10 | IFRD1 | 9.27E−11 | 0.514 | 0.218 | 0.057 | 9.037E−07 |
| CSE1L | 2.42E−14 | −0.719 | 0.23 | 0.123 | 2.355E−10 | CD3EAP | 9.32E−11 | 0.663 | 0.197 | 0.039 | 9.082E−07 |
| COPG1 | 2.42E−14 | −0.458 | 0.247 | 0.123 | 2.357E−10 | GNAI3 | 9.41E−11 | 0.274 | 0.23 | 0.083 | 9.173E−07 |
| AKT2 | 2.43E−14 | −0.818 | 0.169 | 0.07 | 2.368E−10 | MOCS2 | 9.43E−11 | 0.757 | 0.241 | 0.07 | 9.186E−07 |
| PLEKHJ1 | 2.46E−14 | −0.609 | 0.209 | 0.127 | 2.396E−10 | NUP160 | 9.65E−11 | 0.890 | 0.16 | 0.022 | 9.405E−07 |
| ZNF787 | 2.48E−14 | −0.954 | 0.135 | 0.066 | 2.418E−10 | NMRK1 | 9.99E−11 | 0.817 | 0.216 | 0.053 | 9.734E−07 |
| MARS | 2.67E−14 | −0.263 | 0.213 | 0.075 | 2.601E−10 | HIBCH | 1.02E−10 | 0.296 | 0.189 | 0.053 | 9.954E−07 |
| FOXP1 | 2.67E−14 | −0.487 | 0.219 | 0.092 | 2.604E−10 | SUPT20H | 1.02E−10 | 0.526 | 0.221 | 0.066 | 9.980E−07 |
| LPAR6 | 2.73E−14 | −1.314 | 0.112 | 0.092 | 2.659E−10 | MSH2 | 1.12E−10 | 0.427 | 0.225 | 0.066 | 1.087E−06 |
| LRRC42 | 2.86E−14 | −0.315 | 0.157 | 0.031 | 2.790E−10 | PCNA | 1.12E−10 | 0.388 | 0.218 | 0.053 | 1.095E−06 |
| ARF6 | 3.04E−14 | −0.629 | 0.162 | 0.075 | 2.958E−10 | SNX1 | 1.14E−10 | 0.761 | 0.198 | 0.039 | 1.112E−06 |
| TUSC2 | 3.10E−14 | −0.634 | 0.168 | 0.07 | 3.019E−10 | RRM2B | 1.15E−10 | 0.374 | 0.186 | 0.057 | 1.116E−06 |
| ZNF280D | 3.13E−14 | −0.291 | 0.242 | 0.079 | 3.053E−10 | TBK1 | 1.20E−10 | 0.254 | 0.188 | 0.044 | 1.170E−06 |
| PAIP2B | 3.14E−14 | −0.456 | 0.172 | 0.057 | 3.061E−10 | PRRG4 | 1.24E−10 | 0.498 | 0.244 | 0.07 | 1.210E−06 |
| SNAPC5 | 3.15E−14 | −0.412 | 0.133 | 0.039 | 3.072E−10 | ENPP5 | 1.32E−10 | 0.794 | 0.116 | 0.013 | 1.282E−06 |
| PSMA5 | 3.23E−14 | −0.250 | 0.247 | 0.092 | 3.146E−10 | UBE2D1 | 1.35E−10 | 0.886 | 0.197 | 0.035 | 1.319E−06 |
| CCDC23 | 3.32E−14 | −0.292 | 0.191 | 0.061 | 3.238E−10 | DOCK7 | 1.40E−10 | 0.291 | 0.212 | 0.061 | 1.368E−06 |
| TMEM134 | 3.39E−14 | −0.537 | 0.174 | 0.07 | 3.304E−10 | SLC41A1 | 1.48E−10 | 0.285 | 0.147 | 0.035 | 1.447E−06 |
| TRIM38 | 3.50E−14 | −0.397 | 0.172 | 0.044 | 3.409E−10 | CUL4B | 1.53E−10 | 0.473 | 0.168 | 0.048 | 1.492E−06 |
| SRSF6 | 3.65E−14 | −0.468 | 0.185 | 0.053 | 3.557E−10 | LTV1 | 1.58E−10 | 0.252 | 0.212 | 0.083 | 1.535E−06 |
| AC013394.2 | 3.70E−14 | −0.295 | 0.157 | 0.035 | 3.605E−10 | GPC1 | 1.63E−10 | 0.319 | 0.136 | 0.022 | 1.589E−06 |
| FOXO3 | 3.74E−14 | −0.778 | 0.177 | 0.092 | 3.644E−10 | UPP1 | 1.71E−10 | 0.728 | 0.177 | 0.031 | 1.664E−06 |
| C4orf48 | 3.87E−14 | −0.413 | 0.157 | 0.066 | 3.767E−10 | AMOTL2 | 1.80E−10 | 0.370 | 0.159 | 0.026 | 1.755E−06 |
| SAFB | 4.01E−14 | −0.278 | 0.245 | 0.101 | 3.906E−10 | ZNF260 | 1.87E−10 | 0.279 | 0.162 | 0.053 | 1.820E−06 |
| AP3D1 | 4.27E−14 | −0.309 | 0.259 | 0.096 | 4.164E−10 | C18orf56 | 1.90E−10 | 0.572 | 0.182 | 0.039 | 1.855E−06 |
| ELOF1 | 4.33E−14 | −0.751 | 0.145 | 0.057 | 4.222E−10 | CLPX | 1.93E−10 | 0.328 | 0.186 | 0.053 | 1.881E−06 |
| RNF187 | 4.34E−14 | −0.761 | 0.159 | 0.075 | 4.226E−10 | MTMR6 | 1.93E−10 | 0.387 | 0.206 | 0.057 | 1.881E−06 |
| RNF5 | 4.48E−14 | −0.415 | 0.169 | 0.057 | 4.366E−10 | C2orf47 | 1.94E−10 | 0.388 | 0.159 | 0.022 | 1.887E−06 |
| NXT1 | 4.63E−14 | −0.521 | 0.171 | 0.048 | 4.517E−10 | NR3C1 | 1.94E−10 | 0.341 | 0.177 | 0.048 | 1.894E−06 |
| MKKS | 4.68E−14 | −0.357 | 0.189 | 0.07 | 4.556E−10 | YEATS4 | 2.02E−10 | 0.969 | 0.194 | 0.039 | 1.971E−06 |
| POLR3C | 4.78E−14 | −0.408 | 0.228 | 0.096 | 4.661E−10 | ZFC3H1 | 2.06E−10 | 0.663 | 0.169 | 0.022 | 2.011E−06 |
| TCOF1 | 5.16E−14 | −0.515 | 0.206 | 0.07 | 5.029E−10 | TLK1 | 2.13E−10 | 0.286 | 0.194 | 0.075 | 2.074E−06 |
| SBNO1 | 5.21E−14 | −0.477 | 0.268 | 0.145 | 5.076E−10 | FYTTD1 | 2.19E−10 | 0.265 | 0.238 | 0.11 | 2.134E−06 |
| RBM6 | 5.51E−14 | −0.264 | 0.182 | 0.075 | 5.366E−10 | CRY1 | 2.24E−10 | 0.281 | 0.147 | 0.022 | 2.183E−06 |
| PYCARD | 5.63E−14 | −0.304 | 0.157 | 0.048 | 5.482E−10 | SEC14L1 | 2.27E−10 | 0.338 | 0.248 | 0.105 | 2.213E−06 |
| MTAP | 5.83E−14 | −0.395 | 0.216 | 0.11 | 5.677E−10 | USP37 | 2.38E−10 | 0.402 | 0.157 | 0.031 | 2.315E−06 |
| PIN4 | 5.89E−14 | −0.502 | 0.185 | 0.061 | 5.735E−10 | FAM102A | 2.50E−10 | 0.360 | 0.179 | 0.044 | 2.433E−06 |
| TDG | 5.90E−14 | −0.740 | 0.153 | 0.066 | 5.753E−10 | RCN2 | 2.50E−10 | 0.586 | 0.209 | 0.048 | 2.436E−06 |
| SP110 | 6.17E−14 | −0.311 | 0.242 | 0.11 | 6.009E−10 | TMEM57 | 2.62E−10 | 0.795 | 0.195 | 0.053 | 2.555E−06 |
| TSFM | 6.34E−14 | −0.284 | 0.151 | 0.031 | 6.180E−10 | NXT2 | 2.68E−10 | 0.588 | 0.175 | 0.035 | 2.607E−06 |
| KHDRBS3 | 6.60E−14 | −0.584 | 0.189 | 0.07 | 6.430E−10 | AKIP1 | 2.68E−10 | 0.258 | 0.182 | 0.048 | 2.615E−06 |
| EIF4A1 | 6.84E−14 | −0.625 | 0.16 | 0.079 | 6.668E−10 | PDE1C | 2.76E−10 | 0.538 | 0.153 | 0.031 | 2.692E−06 |
| DAPK3 | 7.72E−14 | −0.684 | 0.159 | 0.079 | 7.527E−10 | AHCYL1 | 2.94E−10 | 0.296 | 0.175 | 0.048 | 2.862E−06 |
| PPP2R2A | 8.70E−14 | −0.502 | 0.195 | 0.079 | 8.482E−10 | DNAJC13 | 3.00E−10 | 0.310 | 0.168 | 0.039 | 2.925E−06 |
| GPATCH11 | 9.05E−14 | −0.723 | 0.13 | 0.031 | 8.818E−10 | PLEKHA1 | 3.07E−10 | 0.469 | 0.206 | 0.057 | 2.992E−06 |
| DMAP1 | 9.15E−14 | −0.350 | 0.218 | 0.092 | 8.916E−10 | SLMAP | 3.09E−10 | 0.410 | 0.216 | 0.066 | 3.013E−06 |
| EPB41 | 9.37E−14 | −0.327 | 0.198 | 0.079 | 9.131E−10 | MAP4K3 | 3.18E−10 | 0.723 | 0.201 | 0.048 | 3.094E−06 |
| WHSCI | 9.86E−14 | −0.418 | 0.222 | 0.079 | 9.605E−10 | CCDC125 | 3.21E−10 | 0.392 | 0.138 | 0.018 | 3.124E−06 |
| MVB12A | 9.88E−14 | −0.649 | 0.135 | 0.044 | 9.632E−10 | IRF1 | 3.38E−10 | 0.305 | 0.215 | 0.057 | 3.298E−06 |
| ZNF281 | 9.89E−14 | −0.432 | 0.239 | 0.096 | 9.635E−10 | GPALPP1 | 3.39E−10 | 0.491 | 0.248 | 0.088 | 3.304E−06 |
| PTEN | 1.09E−13 | −0.390 | 0.185 | 0.07 | 1.058E−09 | OTUD3 | 3.42E−10 | 0.729 | 0.115 | 0.013 | 3.328E−06 |
| ANKRD36 | 1.10E−13 | −0.446 | 0.169 | 0.053 | 1.069E−09 | MUT | 3.44E−10 | 0.382 | 0.165 | 0.039 | 3.352E−06 |
| PRKRIP1 | 1.34E−13 | −0.261 | 0.18 | 0.022 | 1.305E−09 | RPS12 | 3.52E−10 | 0.306 | 0.989 | 0.904 | 3.427E−06 |
| MGAT4B | 1.41E−13 | −0.637 | 0.151 | 0.066 | 1.372E−09 | PDE4DIP | 3.52E−10 | 0.308 | 0.183 | 0.044 | 3.433E−06 |
| NELFB | 1.42E−13 | −0.907 | 0.138 | 0.07 | 1.387E−09 | RPAIN | 3.56E−10 | 0.530 | 0.156 | 0.022 | 3.470E−06 |
| VKORC1L1 | 1.53E−13 | −0.377 | 0.248 | 0.114 | 1.489E−09 | CLCC1 | 3.62E−10 | 0.519 | 0.123 | 0.018 | 3.525E−06 |
| SMARCC2 | 1.57E−13 | −0.388 | 0.227 | 0.096 | 1.525E−09 | SNX13 | 3.64E−10 | 0.287 | 0.248 | 0.092 | 3.545E−06 |
| UCHL3 | 1.72E−13 | −0.474 | 0.154 | 0.061 | 1.680E−09 | NUP214 | 3.69E−10 | 0.357 | 0.177 | 0.039 | 3.600E−06 |
| SH3PXD2B | 1.83E−13 | −0.329 | 0.186 | 0.057 | 1.779E−09 | TET3 | 3.72E−10 | 0.344 | 0.183 | 0.044 | 3.624E−06 |
| DLEU1 | 1.96E−13 | −0.520 | 0.133 | 0.039 | 1.913E−09 | MED31 | 3.74E−10 | 1.258 | 0.145 | 0.009 | 3.647E−06 |
| DHFR | 2.06E−13 | −0.663 | 0.109 | 0.035 | 2.005E−09 | MED1 | 3.79E−10 | 0.284 | 0.18 | 0.035 | 3.693E−06 |
| FLYWCH2 | 2.07E−13 | −0.822 | 0.103 | 0.044 | 2.022E−09 | KIAA1524 | 3.80E−10 | 0.369 | 0.169 | 0.044 | 3.705E−06 |
| MAPK6 | 2.18E−13 | −0.514 | 0.206 | 0.075 | 2.123E−09 | C16orf87 | 3.99E−10 | 0.358 | 0.172 | 0.048 | 3.888E−06 |
| DDX6 | 2.52E−13 | −0.527 | 0.247 | 0.171 | 2.455E−09 | KCNQ1OT1 | 4.16E−10 | 0.454 | 0.195 | 0.044 | 4.055E−06 |
| PDCL | 2.55E−13 | −0.315 | 0.153 | 0.053 | 2.487E−09 | CCDC130 | 4.26E−10 | 0.781 | 0.157 | 0.018 | 4.154E−06 |
| MIB2 | 2.56E−13 | −0.474 | 0.144 | 0.035 | 2.491E−09 | MGA | 4.44E−10 | 0.321 | 0.126 | 0.022 | 4.327E−06 |
| TCF3 | 2.66E−13 | −0.550 | 0.215 | 0.105 | 2.592E−09 | SRPR | 4.82E−10 | 0.336 | 0.189 | 0.048 | 4.696E−06 |
| FANCA | 2.67E−13 | −0.510 | 0.182 | 0.07 | 2.605E−09 | MKLN1 | 5.08E−10 | 0.958 | 0.183 | 0.031 | 4.953E−06 |
| TERF2IP | 3.01E−13 | −0.585 | 0.191 | 0.088 | 2.929E−09 | VPS13A | 5.15E−10 | 0.386 | 0.21 | 0.061 | 5.023E−06 |
| SCYL1 | 3.09E−13 | −0.604 | 0.153 | 0.053 | 3.014E−09 | C9orf41 | 5.84E−10 | 0.447 | 0.163 | 0.039 | 5.692E−06 |
| MTHFD2L | 3.09E−13 | −0.287 | 0.195 | 0.053 | 3.015E−09 | BNIP1 | 5.96E−10 | 0.351 | 0.198 | 0.053 | 5.808E−06 |
| MCM7 | 3.13E−13 | −0.324 | 0.175 | 0.066 | 3.055E−09 | RPLP1 | 6.41E−10 | 0.341 | 0.998 | 0.956 | 6.251E−06 |
| NUFIP1 | 3.23E−13 | −1.018 | 0.116 | 0.057 | 3.148E−09 | ZNF254 | 6.52E−10 | 0.498 | 0.147 | 0.018 | 6.355E−06 |
| KIAA0907 | 3.27E−13 | −0.739 | 0.145 | 0.061 | 3.184E−09 | AHR | 6.68E−10 | 0.270 | 0.23 | 0.088 | 6.508E−06 |
| PDCD7 | 3.27E−13 | −1.017 | 0.106 | 0.031 | 3.187E−09 | APOO | 7.05E−10 | 0.270 | 0.153 | 0.031 | 6.866E−06 |
| CIAPIN1 | 3.27E−13 | −0.878 | 0.189 | 0.088 | 3.191E−09 | DDAH1 | 7.18E−10 | 0.378 | 0.183 | 0.057 | 6.994E−06 |
| ETV6 | 3.39E−13 | −0.469 | 0.185 | 0.079 | 3.306E−09 | FBP1 | 7.21E−10 | 0.703 | 0.132 | 0.022 | 7.023E−06 |
| ZMIZ2 | 3.63E−13 | −0.659 | 0.154 | 0.07 | 3.535E−09 | MCM6 | 7.71E−10 | 0.347 | 0.216 | 0.07 | 7.512E−06 |
| YIPF1 | 3.68E−13 | −0.981 | 0.123 | 0.066 | 3.584E−09 | KLHL7 | 7.76E−10 | 1.026 | 0.171 | 0.026 | 7.564E−06 |
| CYTH3 | 3.68E−13 | −0.458 | 0.215 | 0.083 | 3.586E−09 | PEX11A | 7.76E−10 | 0.727 | 0.127 | 0.022 | 7.566E−06 |
| SLC7A5 | 3.88E−13 | −0.357 | 0.188 | 0.066 | 3.778E−09 | PMAIP1 | 7.84E−10 | 0.560 | 0.312 | 0.132 | 7.643E−06 |
| ZC3H7A | 3.90E−13 | −0.349 | 0.209 | 0.066 | 3.805E−09 | FAM98B | 8.89E−10 | 0.390 | 0.242 | 0.088 | 8.666E−06 |
| POLK | 3.97E−13 | −0.830 | 0.154 | 0.079 | 3.868E−09 | PGP | 8.95E−10 | 0.341 | 0.165 | 0.031 | 8.726E−06 |
| CTPS1 | 4.00E−13 | −0.319 | 0.212 | 0.075 | 3.901E−09 | RILPL2 | 9.18E−10 | 0.524 | 0.135 | 0.018 | 8.947E−06 |
| RMRP | 4.14E−13 | −0.568 | 0.182 | 0.092 | 4.039E−09 | TRAF3IP1 | 9.23E−10 | 0.637 | 0.183 | 0.048 | 8.990E−06 |
| SNX5 | 4.23E−13 | −0.393 | 0.216 | 0.101 | 4.125E−09 | ELMOD2 | 9.70E−10 | 0.607 | 0.126 | 0.013 | 9.455E−06 |
| CLDN11 | 4.69E−13 | −0.588 | 0.156 | 0.066 | 4.569E−09 | TRIM37 | 9.71E−10 | 0.331 | 0.18 | 0.048 | 9.467E−06 |
| NOC2L | 4.73E−13 | −0.400 | 0.154 | 0.053 | 4.608E−09 | ORC4 | 9.89E−10 | 0.537 | 0.192 | 0.039 | 9.634E−06 |
| STK38L | 4.82E−13 | −0.735 | 0.159 | 0.101 | 4.698E−09 | EXOC6 | 1.00E−09 | 0.583 | 0.13 | 0.018 | 9.748E−06 |
| FEN1 | 5.00E−13 | −0.646 | 0.15 | 0.07 | 4.869E−09 | LARP4 | 1.01E−09 | 0.334 | 0.238 | 0.088 | 9.812E−06 |
| POLD3 | 5.09E−13 | −0.409 | 0.177 | 0.07 | 4.958E−09 | MICU1 | 1.03E−09 | 0.807 | 0.215 | 0.053 | 1.005E−05 |
| TTC4 | 5.32E−13 | −0.505 | 0.106 | 0.022 | 5.186E−09 | NEDD4L | 1.04E−09 | 0.612 | 0.186 | 0.039 | 1.010E−05 |
| NCOR1 | 5.48E−13 | −0.611 | 0.209 | 0.096 | 5.337E−09 | SKAP2 | 1.04E−09 | 0.261 | 0.142 | 0.026 | 1.017E−05 |
| RFC3 | 5.54E−13 | −0.315 | 0.298 | 0.158 | 5.401E−09 | ST6GALNAC2 | 1.07E−09 | 0.737 | 0.163 | 0.031 | 1.041E−05 |
| NOTCH1 | 5.55E−13 | −0.448 | 0.169 | 0.066 | 5.406E−09 | AHI1 | 1.09E−09 | 0.419 | 0.16 | 0.035 | 1.065E−05 |
| AGPAT1 | 5.61E−13 | −0.997 | 0.112 | 0.066 | 5.472E−09 | CAB39 | 1.10E−09 | 0.322 | 0.219 | 0.088 | 1.068E−05 |
| CECR5 | 5.67E−13 | −0.356 | 0.144 | 0.031 | 5.521E−09 | LEO1 | 1.15E−09 | 0.266 | 0.186 | 0.061 | 1.125E−05 |
| IFI35 | 6.12E−13 | −0.865 | 0.115 | 0.057 | 5.961E−09 | FNBP4 | 1.19E−09 | 0.820 | 0.203 | 0.061 | 1.161E−05 |
| BCL7B | 6.15E−13 | −0.284 | 0.138 | 0.031 | 5.989E−09 | ZBTB21 | 1.24E−09 | 0.749 | 0.2 | 0.048 | 1.205E−05 |
| ADAM15 | 6.30E−13 | −0.558 | 0.203 | 0.101 | 6.138E−09 | GON4L | 1.27E−09 | 0.513 | 0.172 | 0.031 | 1.237E−05 |
| MRRF | 6.58E−13 | −0.271 | 0.163 | 0.048 | 6.407E−09 | BTN2A1 | 1.29E−09 | 0.399 | 0.156 | 0.035 | 1.257E−05 |
| TMEM11 | 6.88E−13 | −0.502 | 0.182 | 0.061 | 6.708E−09 | CUL5 | 1.32E−09 | 0.512 | 0.18 | 0.044 | 1.289E−05 |
| STAP2 | 7.16E−13 | −0.589 | 0.165 | 0.075 | 6.976E−09 | RBM5 | 1.34E−09 | 0.405 | 0.177 | 0.044 | 1.302E−05 |
| CREB1 | 7.67E−13 | −0.886 | 0.16 | 0.079 | 7.475E−09 | CCDC132 | 1.34E−09 | 0.453 | 0.144 | 0.022 | 1.307E−05 |
| CSNK1G2 | 7.69E−13 | −0.285 | 0.182 | 0.039 | 7.497E−09 | CTA- | 1.42E−09 | 0.265 | 0.142 | 0.031 | 1.381E−05 |
| | | | | | | 293F17.1 | | | | | |
| EPB41L2 | 7.93E−13 | −0.527 | 0.262 | 0.132 | 7.727E−09 | CTD- | 1.42E−09 | 0.328 | 0.145 | 0.022 | 1.385E−05 |
| | | | | | | 2228K2.5 | | | | | |
| CSNK2A1 | 8.05E−13 | −0.367 | 0.195 | 0.079 | 7.843E−09 | NAALADL2 | 1.46E−09 | 0.326 | 0.186 | 0.079 | 1.419E−05 |
| WHSC1L1 | 8.64E−13 | −0.518 | 0.165 | 0.075 | 8.421E−09 | CUEDC2 | 1.46E−09 | 0.325 | 0.175 | 0.048 | 1.420E−05 |
| TMTC3 | 8.78E−13 | −0.284 | 0.194 | 0.066 | 8.554E−09 | CDKN1B | 1.46E−09 | 0.449 | 0.201 | 0.075 | 1.426E−05 |
| KIAA1430 | 8.81E−13 | −0.643 | 0.177 | 0.088 | 8.589E−09 | CDC40 | 1.49E−09 | 0.359 | 0.212 | 0.079 | 1.450E−05 |
| TMEM179B | 8.99E−13 | −0.589 | 0.172 | 0.083 | 8.760E−09 | APPBP2 | 1.52E−09 | 0.419 | 0.203 | 0.053 | 1.477E−05 |
| RPL13 | 9.03E−13 | −0.398 | 0.998 | 0.961 | 8.798E−09 | VDAC3 | 1.53E−09 | 0.274 | 0.175 | 0.053 | 1.486E−05 |
| BET1L | 9.40E−13 | −0.502 | 0.101 | 0.022 | 9.158E−09 | EAPP | 1.53E−09 | 0.712 | 0.238 | 0.105 | 1.492E−05 |
| SLC4A1AP | 9.40E−13 | −0.309 | 0.231 | 0.092 | 9.159E−09 | CREB3L2 | 1.57E−09 | 0.259 | 0.172 | 0.044 | 1.532E−05 |
| SRRT | 9.64E−13 | −0.299 | 0.216 | 0.092 | 9.391E−09 | ORC2 | 1.62E−09 | 0.553 | 0.104 | 0.013 | 1.576E−05 |
| AAGAB | 1.03E−12 | −0.448 | 0.159 | 0.044 | 9.997E−09 | C6orf203 | 1.65E−09 | 0.277 | 0.139 | 0.022 | 1.610E−05 |
| CLPTM1 | 1.03E−12 | −0.578 | 0.166 | 0.066 | 1.003E−08 | APMAP | 1.73E−09 | 0.701 | 0.174 | 0.035 | 1.685E−05 |
| AIP | 1.09E−12 | −0.717 | 0.141 | 0.061 | 1.059E−08 | APTX | 1.86E−09 | 0.419 | 0.171 | 0.057 | 1.808E−05 |
| COPS7B | 1.10E−12 | −0.571 | 0.144 | 0.048 | 1.072E−08 | USP46 | 1.86E−09 | 0.392 | 0.136 | 0.018 | 1.811E−05 |
| NDUFAF3 | 1.18E−12 | −0.539 | 0.12 | 0.035 | 1.151E−08 | ZFYVE16 | 1.87E−09 | 0.747 | 0.12 | 0.009 | 1.824E−05 |
| KEAP1 | 1.21E−12 | −0.318 | 0.191 | 0.075 | 1.183E−08 | AGPAT5 | 1.88E−09 | 0.616 | 0.166 | 0.031 | 1.831E−05 |
| RBM10 | 1.26E−12 | −0.274 | 0.179 | 0.039 | 1.226E−08 | MYO9A | 1.89E−09 | 0.789 | 0.148 | 0.018 | 1.846E−05 |
| AKIRIN2 | 1.28E−12 | −0.409 | 0.21 | 0.088 | 1.246E−08 | ASPH | 1.93E−09 | 0.562 | 0.197 | 0.053 | 1.885E−05 |
| BAG6 | 1.35E−12 | −0.783 | 0.165 | 0.061 | 1.313E−08 | RRAGC | 1.97E−09 | 0.377 | 0.177 | 0.035 | 1.920E−05 |
| MPV17 | 1.35E−12 | −0.545 | 0.16 | 0.053 | 1.316E−08 | ERCC6L2 | 1.99E−09 | 0.812 | 0.142 | 0.013 | 1.941E−05 |
| METTL13 | 1.38E−12 | −0.407 | 0.209 | 0.096 | 1.346E−08 | CSTF1 | 2.01E−09 | 0.400 | 0.169 | 0.035 | 1.963E−05 |
| RDX | 1.39E−12 | −0.269 | 0.242 | 0.118 | 1.352E−08 | BAZ2A | 2.10E−09 | 0.309 | 0.174 | 0.048 | 2.042E−05 |
| SND1 | 1.44E−12 | −0.626 | 0.18 | 0.079 | 1.401E−08 | ATXN2 | 2.17E−09 | 0.631 | 0.188 | 0.053 | 2.110E−05 |
| PDS5B | 1.45E−12 | −0.471 | 0.238 | 0.127 | 1.413E−08 | GTF3C4 | 2.22E−09 | 0.613 | 0.204 | 0.057 | 2.167E−05 |
| MT-ND2 | 1.58E−12 | −0.531 | 0.98 | 0.908 | 1.535E−08 | ALKBH5 | 2.23E−09 | 1.174 | 0.133 | 0.009 | 2.173E−05 |
| LYRM1 | 1.62E−12 | −0.369 | 0.175 | 0.061 | 1.575E−08 | UBR4 | 2.28E−09 | 0.917 | 0.185 | 0.044 | 2.221E−05 |
| TXNDC16 | 1.70E−12 | −0.293 | 0.151 | 0.044 | 1.660E−08 | PRUNE | 2.28E−09 | 0.386 | 0.126 | 0.031 | 2.227E−05 |
| LZTS2 | 1.75E−12 | −1.072 | 0.107 | 0.061 | 1.708E−08 | BRD1 | 2.42E−09 | 1.018 | 0.147 | 0.018 | 2.362E−05 |
| GNA13 | 1.81E−12 | −0.607 | 0.241 | 0.127 | 1.760E−08 | CEP44 | 2.47E−09 | 0.692 | 0.135 | 0.022 | 2.407E−05 |
| DLGAP4 | 1.82E−12 | −0.290 | 0.203 | 0.088 | 1.778E−08 | RASEF | 2.53E−09 | 0.584 | 0.197 | 0.053 | 2.466E−05 |
| MSRB2 | 1.89E−12 | −0.766 | 0.165 | 0.079 | 1.839E−08 | TRIP6 | 2.55E−09 | 0.322 | 0.156 | 0.026 | 2.486E−05 |
| PLEKHB2 | 1.94E−12 | −0.664 | 0.166 | 0.07 | 1.889E−08 | BTG2 | 2.58E−09 | 0.376 | 0.138 | 0.026 | 2.517E−05 |
| CNOT6L | 1.95E−12 | −0.433 | 0.135 | 0.057 | 1.900E−08 | UNC13D | 2.66E−09 | 1.010 | 0.121 | 0.004 | 2.595E−05 |
| PDZD11 | 1.99E−12 | −0.963 | 0.123 | 0.079 | 1.937E−08 | SLC4A7 | 2.70E−09 | 0.275 | 0.206 | 0.075 | 2.631E−05 |
| AKT3 | 2.00E−12 | −0.357 | 0.213 | 0.092 | 1.947E−08 | ATMIN | 2.78E−09 | 0.368 | 0.159 | 0.035 | 2.707E−05 |
| GNA11 | 2.04E−12 | −0.425 | 0.172 | 0.066 | 1.989E−08 | ECT2 | 2.82E−09 | 0.349 | 0.28 | 0.154 | 2.750E−05 |
| HPS1 | 2.17E−12 | −0.524 | 0.175 | 0.061 | 2.112E−08 | MDN1 | 2.94E−09 | 0.750 | 0.177 | 0.044 | 2.868E−05 |
| SNRNP48 | 2.21E−12 | −0.345 | 0.182 | 0.053 | 2.156E−08 | TATDN3 | 2.96E−09 | 0.295 | 0.151 | 0.022 | 2.885E−05 |
| DEPTOR | 2.22E−12 | −0.347 | 0.183 | 0.079 | 2.159E−08 | JAK2 | 3.04E−09 | 0.578 | 0.12 | 0.018 | 2.961E−05 |
| RNH1 | 2.22E−12 | −0.475 | 0.157 | 0.048 | 2.167E−08 | ZDHHC16 | 3.10E−09 | 0.271 | 0.145 | 0.031 | 3.024E−05 |
| CEP89 | 2.26E−12 | −0.456 | 0.162 | 0.053 | 2.204E−08 | ZNF669 | 3.11E−09 | 0.579 | 0.203 | 0.061 | 3.031E−05 |
| PPIF | 2.29E−12 | −0.462 | 0.189 | 0.075 | 2.233E−08 | MGME1 | 3.19E−09 | 0.614 | 0.126 | 0.013 | 3.105E−05 |
| USP12 | 2.30E−12 | −0.514 | 0.171 | 0.061 | 2.237E−08 | XPA | 3.37E−09 | 1.309 | 0.148 | 0.018 | 3.282E−05 |
| SLC2A4RG | 2.37E−12 | −0.771 | 0.12 | 0.066 | 2.306E−08 | NCS1 | 3.44E−09 | 0.565 | 0.147 | 0.022 | 3.350E−05 |
| RASAL2 | 2.69E−12 | −0.494 | 0.239 | 0.145 | 2.622E−08 | SMARCAD1 | 3.48E−09 | 0.367 | 0.145 | 0.022 | 3.395E−05 |
| MRPS25 | 2.76E−12 | −0.302 | 0.179 | 0.061 | 2.685E−08 | RLIM | 3.72E−09 | 0.675 | 0.154 | 0.026 | 3.627E−05 |
| BACH1 | 2.82E−12 | −0.251 | 0.212 | 0.101 | 2.751E−08 | KIAA0895 | 4.08E−09 | 0.761 | 0.185 | 0.044 | 3.980E−05 |
| CCND3 | 2.96E−12 | −0.448 | 0.174 | 0.066 | 2.885E−08 | AAED1 | 4.21E−09 | 0.915 | 0.159 | 0.026 | 4.106E−05 |
| MRPL2 | 3.13E−12 | −0.967 | 0.107 | 0.048 | 3.050E−08 | LEPROT | 4.35E−09 | 0.656 | 0.145 | 0.022 | 4.240E−05 |
| MRPL49 | 3.20E−12 | −0.358 | 0.138 | 0.035 | 3.119E−08 | ERCC3 | 4.37E−09 | 0.362 | 0.135 | 0.018 | 4.258E−05 |
| DBNL | 3.44E−12 | −0.271 | 0.197 | 0.083 | 3.355E−08 | TDRKH | 4.74E−09 | 0.422 | 0.182 | 0.053 | 4.616E−05 |
| PQLC1 | 3.47E−12 | −0.734 | 0.101 | 0.039 | 3.382E−08 | STK3 | 4.97E−09 | 0.753 | 0.219 | 0.088 | 4.845E−05 |
| TUBA4A | 3.80E−12 | −0.288 | 0.171 | 0.053 | 3.701E−08 | ACTR10 | 4.99E−09 | 0.414 | 0.169 | 0.039 | 4.866E−05 |
| ZCCHC10 | 3.91E−12 | −0.315 | 0.183 | 0.07 | 3.806E−08 | ZRANB1 | 5.42E−09 | 0.598 | 0.172 | 0.035 | 5.278E−05 |
| BRCA2 | 3.94E−12 | −0.328 | 0.185 | 0.061 | 3.843E−08 | USP47 | 5.63E−09 | 0.443 | 0.21 | 0.083 | 5.487E−05 |
| THEM6 | 3.99E−12 | −0.360 | 0.169 | 0.075 | 3.888E−08 | PAQR4 | 5.68E−09 | 0.309 | 0.166 | 0.044 | 5.533E−05 |
| SSBP2 | 4.05E−12 | −0.655 | 0.154 | 0.066 | 3.947E−08 | PPP1R16A | 5.72E−09 | 0.941 | 0.159 | 0.031 | 5.579E−05 |
| SNHG15 | 4.13E−12 | −0.354 | 0.154 | 0.053 | 4.023E−08 | TMEM245 | 5.97E−09 | 0.638 | 0.166 | 0.048 | 5.818E−05 |
| ELK1 | 4.16E−12 | −0.644 | 0.13 | 0.048 | 4.057E−08 | RAB22A | 6.02E−09 | 0.476 | 0.233 | 0.088 | 5.862E−05 |
| CCDC88A | 4.51E−12 | −0.347 | 0.244 | 0.127 | 4.390E−08 | MCM5 | 6.03E−09 | 0.502 | 0.159 | 0.035 | 5.875E−05 |
| GCHFR | 4.58E−12 | −0.505 | 0.159 | 0.057 | 4.462E−08 | LRCH3 | 6.30E−09 | 0.252 | 0.135 | 0.031 | 6.138E−05 |
| DDX41 | 4.71E−12 | −0.518 | 0.171 | 0.057 | 4.593E−08 | LIN9 | 6.39E−09 | 0.421 | 0.195 | 0.057 | 6.224E−05 |
| CAMK2D | 4.80E−12 | −0.329 | 0.236 | 0.11 | 4.677E−08 | RABEP1 | 6.49E−09 | 0.585 | 0.172 | 0.044 | 6.324E−05 |
| ORMDL2 | 4.93E−12 | −0.373 | 0.182 | 0.053 | 4.804E−08 | SUGP2 | 6.50E−09 | 0.377 | 0.165 | 0.031 | 6.332E−05 |
| MOB4 | 4.99E−12 | −0.593 | 0.136 | 0.053 | 4.866E−08 | RNMT | 6.55E−09 | 0.301 | 0.204 | 0.07 | 6.387E−05 |
| LEPROTL1 | 5.19E−12 | −0.817 | 0.171 | 0.07 | 5.053E−08 | TULP3 | 6.64E−09 | 0.269 | 0.182 | 0.057 | 6.474E−05 |
| C19orf48 | 5.34E−12 | −0.479 | 0.141 | 0.039 | 5.201E−08 | ZMYM2 | 6.67E−09 | 0.304 | 0.21 | 0.066 | 6.502E−05 |
| NUP62 | 5.42E−12 | −0.368 | 0.183 | 0.083 | 5.285E−08 | FAM8A1 | 6.80E−09 | 0.297 | 0.12 | 0.022 | 6.627E−05 |
| NF2 | 5.53E−12 | −0.447 | 0.18 | 0.088 | 5.387E−08 | ARHGEF12 | 6.93E−09 | 0.640 | 0.169 | 0.031 | 6.749E−05 |
| REV3L | 5.64E−12 | −0.438 | 0.212 | 0.101 | 5.497E−08 | MOB1B | 7.65E−09 | 0.423 | 0.159 | 0.044 | 7.452E−05 |
| WTAP | 5.81E−12 | −0.285 | 0.213 | 0.083 | 5.666E−08 | TRIB1 | 7.75E−09 | 0.593 | 0.166 | 0.044 | 7.555E−05 |
| PHF19 | 5.84E−12 | −0.514 | 0.159 | 0.061 | 5.695E−08 | ZNF330 | 7.83E−09 | 0.284 | 0.148 | 0.039 | 7.630E−05 |
| M6PR | 5.97E−12 | −0.380 | 0.227 | 0.101 | 5.820E−08 | COMTD1 | 7.86E−09 | 0.337 | 0.16 | 0.039 | 7.658E−05 |
| MRPL15 | 6.00E−12 | −0.251 | 0.192 | 0.044 | 5.843E−08 | RSPRY1 | 8.22E−09 | 0.755 | 0.2 | 0.053 | 8.012E−05 |
| KCTD12 | 6.03E−12 | −0.473 | 0.171 | 0.048 | 5.876E−08 | TXLNB | 8.35E−09 | 0.333 | 0.132 | 0.031 | 8.134E−05 |
| VRK2 | 6.42E−12 | −0.321 | 0.242 | 0.118 | 6.260E−08 | GCFC2 | 8.45E−09 | 0.362 | 0.16 | 0.039 | 8.238E−05 |
| BST2 | 6.45E−12 | −0.322 | 0.216 | 0.132 | 6.281E−08 | C12orf73 | 8.52E−09 | 0.356 | 0.133 | 0.022 | 8.305E−05 |
| DUSP3 | 6.86E−12 | −0.608 | 0.177 | 0.083 | 6.687E−08 | PPFIA1 | 8.57E−09 | 0.792 | 0.15 | 0.026 | 8.354E−05 |
| DNAJC21 | 6.95E−12 | −0.467 | 0.206 | 0.079 | 6.775E−08 | TRMT11 | 8.98E−09 | 0.642 | 0.169 | 0.039 | 8.755E−05 |
| SNX14 | 7.02E−12 | −0.351 | 0.215 | 0.083 | 6.839E−08 | ADNP2 | 9.38E−09 | 0.531 | 0.126 | 0.031 | 9.137E−05 |
| TSC22D4 | 7.11E−12 | −0.528 | 0.13 | 0.035 | 6.930E−08 | DUSP10 | 9.49E−09 | 0.267 | 0.165 | 0.044 | 9.249E−05 |
| C9orf114 | 7.41E−12 | −0.714 | 0.145 | 0.066 | 7.217E−08 | MAFG | 9.59E−09 | 0.358 | 0.215 | 0.07 | 9.346E−05 |
| RNF130 | 7.69E−12 | −0.330 | 0.191 | 0.096 | 7.497E−08 | CITED2 | 1.01E−08 | 0.473 | 0.194 | 0.053 | 9.870E−05 |
| PMPCA | 7.76E−12 | −0.269 | 0.154 | 0.048 | 7.563E−08 | FAM53C | 1.02E−08 | 0.837 | 0.103 | 0.009 | 9.975E−05 |
| HNRNPUL2 | 7.96E−12 | −0.836 | 0.107 | 0.044 | 7.756E−08 | FBXO18 | 1.09E−08 | 0.628 | 0.141 | 0.035 | 1.061E−04 |
| TBC1D9B | 8.49E−12 | −0.622 | 0.142 | 0.066 | 8.271E−08 | ERI2 | 1.14E−08 | 0.521 | 0.144 | 0.022 | 1.109E−04 |
| DNAJC11 | 8.57E−12 | −0.538 | 0.175 | 0.048 | 8.353E−08 | KIAA1279 | 1.16E−08 | 0.448 | 0.126 | 0.022 | 1.134E−04 |
| SGTA | 8.68E−12 | −0.670 | 0.147 | 0.066 | 8.458E−08 | SGCB | 1.17E−08 | 0.372 | 0.169 | 0.048 | 1.139E−04 |
| SH3RF1 | 8.96E−12 | −0.527 | 0.25 | 0.14 | 8.731E−08 | TRMU | 1.21E−08 | 0.528 | 0.144 | 0.026 | 1.182E−04 |
| SLC35A4 | 9.51E−12 | −0.271 | 0.159 | 0.079 | 9.265E−08 | CDK8 | 1.22E−08 | 0.884 | 0.147 | 0.026 | 1.192E−04 |
| MGAT1 | 1.01E−11 | −0.442 | 0.177 | 0.066 | 9.856E−08 | TINCR | 1.24E−08 | 0.356 | 0.101 | 0.018 | 1.205E−04 |
| TSPYL1 | 1.06E−11 | −0.532 | 0.153 | 0.083 | 1.036E−07 | RAD51C | 1.25E−08 | 0.705 | 0.18 | 0.039 | 1.218E−04 |
| MPST | 1.07E−11 | −0.276 | 0.154 | 0.035 | 1.047E−07 | MTA3 | 1.28E−08 | 0.360 | 0.169 | 0.048 | 1.243E−04 |
| DNASE1L1 | 1.11E−11 | −0.252 | 0.15 | 0.031 | 1.082E−07 | KLHL24 | 1.28E−08 | 0.553 | 0.182 | 0.048 | 1.247E−04 |
| FOLR1 | 1.12E−11 | −0.564 | 0.139 | 0.053 | 1.095E−07 | SLC1A3 | 1.29E−08 | 0.325 | 0.139 | 0.031 | 1.257E−04 |
| RFXANK | 1.17E−11 | −0.377 | 0.177 | 0.075 | 1.139E−07 | ACACA | 1.44E−08 | 0.481 | 0.168 | 0.044 | 1.399E−04 |
| PTPN18 | 1.17E−11 | −0.282 | 0.189 | 0.066 | 1.144E−07 | STX3 | 1.47E−08 | 0.333 | 0.175 | 0.057 | 1.430E−04 |
| PPM1A | 1.18E−11 | −0.361 | 0.201 | 0.066 | 1.154E−07 | ACAP3 | 1.47E−08 | 0.365 | 0.148 | 0.035 | 1.432E−04 |
| PPP1R14C | 1.25E−11 | −0.679 | 0.126 | 0.061 | 1.216E−07 | ETFA | 1.49E−08 | 0.572 | 0.186 | 0.048 | 1.455E−04 |
| PPP5C | 1.25E−11 | −0.389 | 0.163 | 0.044 | 1.220E−07 | LCOR | 1.53E−08 | 0.690 | 0.153 | 0.039 | 1.494E−04 |
| AC005152.2 | 1.28E−11 | −0.304 | 0.171 | 0.07 | 1.244E−07 | TMCO4 | 1.55E−08 | 1.201 | 0.124 | 0.013 | 1.514E−04 |
| DAAM1 | 1.31E−11 | −0.594 | 0.168 | 0.066 | 1.280E−07 | SLC15A2 | 1.56E−08 | 0.251 | 0.126 | 0.031 | 1.524E−04 |
| SCMH1 | 1.40E−11 | −0.437 | 0.145 | 0.048 | 1.369E−07 | MED19 | 1.57E−08 | 0.474 | 0.182 | 0.053 | 1.530E−04 |
| ZMYND8 | 1.41E−11 | −0.494 | 0.154 | 0.07 | 1.377E−07 | NRAS | 1.57E−08 | 0.735 | 0.159 | 0.039 | 1.531E−04 |
| UGCG | 1.49E−11 | −0.592 | 0.166 | 0.066 | 1.453E−07 | YEATS2 | 1.60E−08 | 0.253 | 0.168 | 0.057 | 1.556E−04 |
| MRPS11 | 1.50E−11 | −0.864 | 0.112 | 0.053 | 1.457E−07 | RHNO1 | 1.61E−08 | 0.256 | 0.135 | 0.035 | 1.573E−04 |
| UGGT1 | 1.53E−11 | −0.364 | 0.204 | 0.092 | 1.491E−07 | CEP78 | 1.64E−08 | 0.901 | 0.165 | 0.035 | 1.600E−04 |
| ERCC5 | 1.56E−11 | −0.493 | 0.139 | 0.053 | 1.523E−07 | NHLRC2 | 1.66E−08 | 0.385 | 0.185 | 0.053 | 1.621E−04 |
| TSPAN4 | 1.62E−11 | −0.835 | 0.104 | 0.061 | 1.574E−07 | RIT1 | 1.68E−08 | 0.306 | 0.216 | 0.075 | 1.638E−04 |
| CEP63 | 1.67E−11 | −0.253 | 0.198 | 0.088 | 1.627E−07 | PIK3C3 | 1.71E−08 | 0.724 | 0.11 | 0.013 | 1.669E−04 |
| PHF2 | 1.70E−11 | −0.665 | 0.12 | 0.044 | 1.652E−07 | TNFAIP1 | 1.75E−08 | 0.398 | 0.188 | 0.048 | 1.706E−04 |
| BIRC6 | 1.75E−11 | −0.414 | 0.254 | 0.149 | 1.703E−07 | KAT6B | 1.78E−08 | 0.541 | 0.101 | 0.009 | 1.739E−04 |
| PPP1R11 | 1.81E−11 | −0.415 | 0.12 | 0.039 | 1.767E−07 | OSBPL9 | 1.81E−08 | 0.346 | 0.171 | 0.048 | 1.764E−04 |
| MAN1B1 | 1.85E−11 | −0.313 | 0.172 | 0.061 | 1.807E−07 | SLC35F5 | 1.82E−08 | 0.258 | 0.182 | 0.061 | 1.775E−04 |
| PSMF1 | 1.87E−11 | −0.311 | 0.118 | 0.026 | 1.826E−07 | ZNF195 | 1.85E−08 | 0.435 | 0.197 | 0.066 | 1.802E−04 |
| DEDD | 1.99E−11 | −0.869 | 0.101 | 0.048 | 1.936E−07 | PIAS4 | 1.91E−08 | 0.544 | 0.133 | 0.018 | 1.860E−04 |
| CCDC25 | 2.00E−11 | −0.549 | 0.135 | 0.066 | 1.950E−07 | DNAJC24 | 1.98E−08 | 0.514 | 0.142 | 0.031 | 1.926E−04 |
| CSNK2A2 | 2.02E−11 | −0.435 | 0.126 | 0.044 | 1.970E−07 | ASAP2 | 1.98E−08 | 0.945 | 0.138 | 0.022 | 1.927E−04 |
| CENPM | 2.04E−11 | −0.790 | 0.103 | 0.039 | 1.987E−07 | MARVELD3 | 2.05E−08 | 0.499 | 0.132 | 0.026 | 1.995E−04 |
| RNASEH2C | 2.16E−11 | −0.403 | 0.188 | 0.066 | 2.102E−07 | NRN1 | 2.05E−08 | 0.820 | 0.132 | 0.018 | 2.001E−04 |
| IMP4 | 2.21E−11 | −0.361 | 0.162 | 0.057 | 2.149E−07 | LINC00511 | 2.13E−08 | 0.598 | 0.168 | 0.035 | 2.079E−04 |
| FOLH1 | 2.25E−11 | −0.447 | 0.142 | 0.044 | 2.197E−07 | COX15 | 2.19E−08 | 0.456 | 0.171 | 0.048 | 2.135E−04 |
| CENPF | 2.29E−11 | −0.254 | 0.478 | 0.461 | 2.230E−07 | CNTLN | 2.26E−08 | 0.566 | 0.148 | 0.026 | 2.207E−04 |
| MSH3 | 2.34E−11 | −0.568 | 0.15 | 0.053 | 2.276E−07 | FAM171B | 2.38E−08 | 0.544 | 0.13 | 0.018 | 2.319E−04 |
| WRB | 2.37E−11 | −0.477 | 0.16 | 0.061 | 2.310E−07 | ZUFSP | 2.38E−08 | 0.723 | 0.169 | 0.035 | 2.320E−04 |
| CSTF2 | 2.39E−11 | −0.328 | 0.183 | 0.079 | 2.333E−07 | NOM1 | 2.42E−08 | 0.559 | 0.132 | 0.013 | 2.356E−04 |
| CCNB1 | 2.43E−11 | −0.294 | 0.234 | 0.197 | 2.367E−07 | COMMD10 | 2.51E−08 | 0.335 | 0.166 | 0.048 | 2.443E−04 |
| HDAC6 | 2.46E−11 | −0.518 | 0.15 | 0.057 | 2.398E−07 | ENPP4 | 2.52E−08 | 0.277 | 0.191 | 0.066 | 2.451E−04 |
| SNX8 | 2.49E−11 | −0.541 | 0.154 | 0.066 | 2.428E−07 | MRPS30 | 2.65E−08 | 1.169 | 0.13 | 0.013 | 2.584E−04 |
| TSC22D2 | 2.57E−11 | −0.389 | 0.219 | 0.114 | 2.501E−07 | RASA1 | 2.72E−08 | 0.385 | 0.13 | 0.018 | 2.647E−04 |
| WIPF2 | 2.60E−11 | −0.403 | 0.13 | 0.031 | 2.537E−07 | LINS | 2.86E−08 | 0.570 | 0.16 | 0.044 | 2.785E−04 |
| DNPEP | 2.72E−11 | −0.769 | 0.11 | 0.048 | 2.653E−07 | PPP4R1 | 2.86E−08 | 0.252 | 0.177 | 0.057 | 2.788E−04 |
| LRRC16A | 2.77E−11 | −0.524 | 0.174 | 0.075 | 2.700E−07 | IL33 | 2.88E−08 | 0.786 | 0.126 | 0.018 | 2.808E−04 |
| PAWR | 2.86E−11 | −0.355 | 0.191 | 0.075 | 2.785E−07 | PPARA | 2.89E−08 | 0.622 | 0.139 | 0.031 | 2.815E−04 |
| BTBD10 | 3.01E−11 | −0.757 | 0.113 | 0.035 | 2.934E−07 | RDH10 | 2.95E−08 | 0.496 | 0.148 | 0.031 | 2.878E−04 |
| C19orf60 | 3.11E−11 | −0.495 | 0.147 | 0.053 | 3.027E−07 | C10orf137 | 3.06E−08 | 0.570 | 0.13 | 0.026 | 2.986E−04 |
| MIOS | 3.15E−11 | −0.254 | 0.145 | 0.039 | 3.065E−07 | MPDZ | 3.06E−08 | 0.316 | 0.15 | 0.039 | 2.986E−04 |
| EI24 | 3.47E−11 | −0.489 | 0.159 | 0.057 | 3.386E−07 | PPWD1 | 3.12E−08 | 1.047 | 0.142 | 0.031 | 3.042E−04 |
| MTHFD1 | 3.49E−11 | −0.649 | 0.174 | 0.096 | 3.398E−07 | VPS8 | 3.13E−08 | 0.435 | 0.169 | 0.057 | 3.050E−04 |
| BTBD3 | 3.58E−11 | −0.258 | 0.168 | 0.057 | 3.486E−07 | PLEC | 3.29E−08 | 0.278 | 0.163 | 0.092 | 3.211E−04 |
| GATAD2B | 3.61E−11 | −0.292 | 0.163 | 0.07 | 3.521E−07 | IER3 | 3.36E−08 | 0.517 | 0.129 | 0.022 | 3.273E−04 |
| IFT80 | 3.73E−11 | −0.305 | 0.12 | 0.022 | 3.634E−07 | TMEM5 | 3.42E−08 | 0.292 | 0.168 | 0.048 | 3.331E−04 |
| TRMT5 | 3.74E−11 | −0.367 | 0.177 | 0.061 | 3.644E−07 | POLR3D | 3.44E−08 | 0.748 | 0.121 | 0.013 | 3.348E−04 |
| TADA3 | 3.74E−11 | −0.679 | 0.136 | 0.061 | 3.648E−07 | WDFY1 | 3.54E−08 | 0.458 | 0.138 | 0.031 | 3.452E−04 |
| MLXIP | 3.75E−11 | −0.389 | 0.186 | 0.07 | 3.658E−07 | TMEM18 | 3.55E−08 | 0.509 | 0.144 | 0.035 | 3.462E−04 |
| ZBTB7A | 3.91E−11 | −0.812 | 0.11 | 0.053 | 3.814E−07 | ZBTB1 | 3.77E−08 | 0.838 | 0.16 | 0.035 | 3.677E−04 |
| GLTPD1 | 3.93E−11 | −0.969 | 0.116 | 0.07 | 3.826E−07 | PPP6R3 | 3.83E−08 | 0.300 | 0.188 | 0.075 | 3.730E−04 |
| SWI5 | 4.02E−11 | −0.620 | 0.133 | 0.048 | 3.915E−07 | TCTN1 | 3.83E−08 | 0.259 | 0.121 | 0.018 | 3.735E−04 |
| TRIM25 | 4.07E−11 | −0.282 | 0.212 | 0.092 | 3.971E−07 | PAFAH1B2 | 3.91E−08 | 0.454 | 0.194 | 0.07 | 3.810E−04 |
| FAM107B | 4.69E−11 | −0.732 | 0.13 | 0.075 | 4.569E−07 | TUBGCP5 | 3.92E−08 | 1.201 | 0.136 | 0.022 | 3.817E−04 |
| C15orf61 | 4.85E−11 | −0.811 | 0.15 | 0.079 | 4.728E−07 | SC5D | 4.04E−08 | 0.663 | 0.162 | 0.035 | 3.936E−04 |
| SKP2 | 4.86E−11 | −0.538 | 0.139 | 0.044 | 4.737E−07 | KIAA1429 | 4.05E−08 | 0.420 | 0.182 | 0.057 | 3.949E−04 |
| UBP1 | 5.05E−11 | −0.255 | 0.186 | 0.061 | 4.921E−07 | DENND6A | 4.11E−08 | 0.764 | 0.142 | 0.022 | 4.006E−04 |
| PAX6 | 5.07E−11 | −0.287 | 0.142 | 0.053 | 4.945E−07 | RP11- | 4.13E−08 | 0.326 | 0.127 | 0.022 | 4.025E−04 |
| | | | | | | 732M18.3 | | | | | |
| NKAP | 5.39E−11 | −0.681 | 0.133 | 0.061 | 5.256E−07 | MED21 | 4.32E−08 | 0.422 | 0.148 | 0.039 | 4.212E−04 |
| G6PC3 | 5.54E−11 | −0.430 | 0.113 | 0.035 | 5.401E−07 | CWF19L1 | 4.44E−08 | 0.649 | 0.126 | 0.022 | 4.329E−04 |
| NNT-AS1 | 5.55E−11 | −0.621 | 0.109 | 0.044 | 5.411E−07 | RBFOX2 | 4.58E−08 | 0.345 | 0.144 | 0.039 | 4.459E−04 |
| MFSD10 | 5.71E−11 | −0.486 | 0.115 | 0.031 | 5.561E−07 | EFCAB2 | 4.72E−08 | 0.292 | 0.127 | 0.022 | 4.598E−04 |
| MRPL45 | 5.88E−11 | −0.630 | 0.132 | 0.061 | 5.726E−07 | TRAPPC10 | 4.79E−08 | 0.380 | 0.142 | 0.022 | 4.666E−04 |
| AGT | 5.88E−11 | −0.254 | 0.147 | 0.039 | 5.727E−07 | TUBGCP4 | 4.80E−08 | 0.646 | 0.135 | 0.022 | 4.678E−04 |
| PTPN3 | 6.00E−11 | −0.585 | 0.113 | 0.048 | 5.843E−07 | MNAT1 | 4.87E−08 | 0.259 | 0.179 | 0.057 | 4.744E−04 |
| SETD5-AS1 | 6.40E−11 | −0.733 | 0.118 | 0.066 | 6.239E−07 | TSPAN17 | 4.88E−08 | 0.857 | 0.106 | 0.004 | 4.757E−04 |
| PRMT5 | 6.43E−11 | −0.489 | 0.151 | 0.057 | 6.262E−07 | ENDOD1 | 5.08E−08 | 0.872 | 0.127 | 0.031 | 4.946E−04 |
| KXD1 | 6.43E−11 | −0.470 | 0.16 | 0.075 | 6.268E−07 | CNOT10 | 5.14E−08 | 0.791 | 0.166 | 0.048 | 5.006E−04 |
| GAA | 6.63E−11 | −0.519 | 0.109 | 0.026 | 6.461E−07 | PTCHD3P1 | 5.24E−08 | 0.312 | 0.183 | 0.061 | 5.111E−04 |
| IWS1 | 6.80E−11 | −0.436 | 0.2 | 0.083 | 6.628E−07 | TRUB1 | 5.34E−08 | 0.423 | 0.151 | 0.031 | 5.208E−04 |
| NOTCH2NL | 6.89E−11 | −0.534 | 0.104 | 0.035 | 6.711E−07 | INPP5F | 5.45E−08 | 0.631 | 0.121 | 0.022 | 5.314E−04 |
| PROSC | 7.12E−11 | −0.416 | 0.169 | 0.061 | 6.936E−07 | NCAPG2 | 5.64E−08 | 0.734 | 0.159 | 0.039 | 5.499E−04 |
| CCDC12 | 7.20E−11 | −0.274 | 0.165 | 0.061 | 7.017E−07 | TDRD7 | 5.67E−08 | 0.272 | 0.168 | 0.044 | 5.524E−04 |
| SURF2 | 7.25E−11 | −0.284 | 0.16 | 0.053 | 7.063E−07 | TAF12 | 5.71E−08 | 0.255 | 0.145 | 0.035 | 5.563E−04 |
| PEPD | 7.27E−11 | −0.584 | 0.165 | 0.079 | 7.086E−07 | RPP40 | 5.93E−08 | 0.942 | 0.156 | 0.044 | 5.775E−04 |
| HIP1 | 7.48E−11 | −0.399 | 0.133 | 0.048 | 7.290E−07 | UTP20 | 6.33E−08 | 0.281 | 0.133 | 0.031 | 6.173E−04 |
| NRP1 | 7.94E−11 | −0.400 | 0.154 | 0.048 | 7.742E−07 | RREB1 | 6.49E−08 | 0.701 | 0.124 | 0.018 | 6.322E−04 |
| ARHGEF10L | 8.13E−11 | −0.404 | 0.159 | 0.07 | 7.923E−07 | APBB2 | 6.57E−08 | 0.711 | 0.121 | 0.013 | 6.402E−04 |
| STK40 | 8.54E−11 | −0.442 | 0.121 | 0.039 | 8.324E−07 | PARP8 | 6.97E−08 | 0.378 | 0.121 | 0.026 | 6.796E−04 |
| BANP | 8.78E−11 | −0.609 | 0.109 | 0.035 | 8.553E−07 | MASTL | 7.03E−08 | 0.507 | 0.136 | 0.026 | 6.851E−04 |
| AKT1 | 8.97E−11 | −0.586 | 0.163 | 0.079 | 8.739E−07 | SERTAD2 | 7.08E−08 | 0.363 | 0.139 | 0.031 | 6.900E−04 |
| SNF8 | 9.23E−11 | −0.413 | 0.174 | 0.066 | 8.992E−07 | RRAGD | 7.20E−08 | 0.966 | 0.12 | 0.013 | 7.017E−04 |
| TMOD1 | 9.24E−11 | −0.366 | 0.197 | 0.083 | 9.004E−07 | PAXBP1 | 7.21E−08 | 0.426 | 0.132 | 0.022 | 7.023E−04 |
| VEGFB | 9.28E−11 | −0.720 | 0.11 | 0.039 | 9.039E−07 | FABP7 | 7.31E−08 | 0.263 | 0.127 | 0.022 | 7.127E−04 |
| KIAA0922 | 9.35E−11 | −0.325 | 0.174 | 0.053 | 9.107E−07 | TRIM4 | 7.32E−08 | 0.467 | 0.15 | 0.048 | 7.137E−04 |
| NCBP2-AS2 | 9.39E−11 | −0.664 | 0.153 | 0.079 | 9.153E−07 | GBE1 | 7.76E−08 | 0.536 | 0.169 | 0.039 | 7.563E−04 |
| NAB1 | 9.42E−11 | −0.574 | 0.175 | 0.057 | 9.178E−07 | DYRK2 | 8.36E−08 | 0.448 | 0.124 | 0.018 | 8.146E−04 |
| CNKSR3 | 9.45E−11 | −0.360 | 0.139 | 0.035 | 9.206E−07 | DTWD1 | 8.56E−08 | 0.552 | 0.135 | 0.035 | 8.341E−04 |
| NBPF1 | 9.97E−11 | −0.619 | 0.171 | 0.092 | 9.713E−07 | HIST1H1B | 8.63E−08 | 1.744 | 0.185 | 0.066 | 8.411E−04 |
| KIAA0430 | 1.01E−10 | −0.705 | 0.116 | 0.053 | 9.843E−07 | ARFGEF2 | 8.69E−08 | 0.354 | 0.186 | 0.061 | 8.467E−04 |
| HSPBP1 | 1.04E−10 | −0.570 | 0.12 | 0.039 | 1.011E−06 | FAM175B | 8.75E−08 | 0.511 | 0.166 | 0.044 | 8.529E−04 |
| FPGS | 1.05E−10 | −0.720 | 0.115 | 0.053 | 1.021E−06 | GNG7 | 8.94E−08 | 0.696 | 0.121 | 0.018 | 8.711E−04 |
| HCFC1 | 1.06E−10 | −0.567 | 0.172 | 0.088 | 1.031E−06 | TLR1 | 8.95E−08 | 0.902 | 0.123 | 0.035 | 8.722E−04 |
| ARF5 | 1.11E−10 | −0.605 | 0.129 | 0.057 | 1.078E−06 | RIC8B | 9.02E−08 | 0.753 | 0.139 | 0.031 | 8.786E−04 |
| DPCD | 1.15E−10 | −0.415 | 0.159 | 0.075 | 1.117E−06 | TP53RK | 9.09E−08 | 0.432 | 0.175 | 0.066 | 8.854E−04 |
| SDF2L1 | 1.15E−10 | −0.576 | 0.124 | 0.039 | 1.120E−06 | GJC1 | 9.43E−08 | 0.752 | 0.113 | 0.013 | 9.188E−04 |
| BRD7 | 1.22E−10 | −0.634 | 0.141 | 0.053 | 1.192E−06 | RP11- | 1.00E−07 | 0.312 | 0.148 | 0.039 | 9.746E−04 |
| | | | | | | 452F19.3 | | | | | |
| ST3GAL4 | 1.23E−10 | −0.274 | 0.163 | 0.079 | 1.202E−06 | SAMD4B | 1.00E−07 | 0.300 | 0.141 | 0.031 | 9.785E−04 |
| RNF44 | 1.28E−10 | −0.563 | 0.113 | 0.035 | 1.243E−06 | ANAPC10 | 1.02E−07 | 0.382 | 0.136 | 0.026 | 9.912E−04 |
| DBF4 | 1.32E−10 | −0.448 | 0.197 | 0.11 | 1.290E−06 | CCDC122 | 1.07E−07 | 1.209 | 0.113 | 0.013 | 1.047E−03 |
| ATP11A | 1.33E−10 | −0.734 | 0.139 | 0.088 | 1.296E−06 | STYXL1 | 1.10E−07 | 0.412 | 0.154 | 0.039 | 1.070E−03 |
| ARID5A | 1.39E−10 | −0.580 | 0.123 | 0.066 | 1.359E−06 | HCCS | 1.10E−07 | 0.357 | 0.165 | 0.044 | 1.074E−03 |
| SLC35C1 | 1.43E−10 | −0.822 | 0.124 | 0.066 | 1.389E−06 | CAB39L | 1.12E−07 | 0.541 | 0.13 | 0.022 | 1.091E−03 |
| AATF | 1.45E−10 | −0.717 | 0.15 | 0.083 | 1.408E−06 | ZNF354A | 1.12E−07 | 0.484 | 0.148 | 0.031 | 1.091E−03 |
| ABT1 | 1.47E−10 | −0.407 | 0.121 | 0.031 | 1.431E−06 | MAP9 | 1.13E−07 | 0.779 | 0.165 | 0.039 | 1.104E−03 |
| RC3H1 | 1.49E−10 | −0.258 | 0.204 | 0.079 | 1.455E−06 | ODF2L | 1.16E−07 | 0.308 | 0.154 | 0.039 | 1.129E−03 |
| DCAF11 | 1.56E−10 | −0.466 | 0.154 | 0.061 | 1.520E−06 | PIGP | 1.16E−07 | 0.418 | 0.129 | 0.022 | 1.135E−03 |
| KIF22 | 1.59E−10 | −0.255 | 0.189 | 0.061 | 1.550E−06 | RBMS1 | 1.18E−07 | 0.289 | 0.135 | 0.026 | 1.147E−03 |
| TMEM230 | 1.68E−10 | −0.446 | 0.138 | 0.053 | 1.633E−06 | PXDC1 | 1.23E−07 | 0.286 | 0.136 | 0.026 | 1.199E−03 |
| TRABD | 1.71E−10 | −0.298 | 0.163 | 0.057 | 1.662E−06 | WASF1 | 1.27E−07 | 0.355 | 0.144 | 0.039 | 1.233E−03 |
| RP11- | 1.75E−10 | −0.558 | 0.132 | 0.066 | 1.709E−06 | KDM4A | 1.33E−07 | 0.466 | 0.112 | 0.013 | 1.292E−03 |
| 145M9.4 | | | | | | | | | | | |
| CYB5B | 1.90E−10 | −0.275 | 0.177 | 0.061 | 1.850E−06 | DECR1 | 1.36E−07 | 0.261 | 0.132 | 0.026 | 1.323E−03 |
| SAFB2 | 2.00E−10 | −0.262 | 0.154 | 0.07 | 1.947E−06 | ZNF622 | 1.38E−07 | 0.333 | 0.147 | 0.048 | 1.342E−03 |
| NAPRT1 | 2.01E−10 | −0.252 | 0.191 | 0.07 | 1.963E−06 | CDC42BPB | 1.40E−07 | 0.332 | 0.145 | 0.044 | 1.362E−03 |
| RFC2 | 2.09E−10 | −0.436 | 0.169 | 0.075 | 2.041E−06 | MAP3K4 | 1.44E−07 | 0.796 | 0.127 | 0.022 | 1.400E−03 |
| USP32 | 2.12E−10 | −0.589 | 0.169 | 0.088 | 2.069E−06 | PCGF1 | 1.44E−07 | 0.434 | 0.106 | 0.013 | 1.404E−03 |
| TAF10 | 2.17E−10 | −0.296 | 0.126 | 0.035 | 2.118E−06 | FOXJ3 | 1.46E−07 | 0.397 | 0.168 | 0.066 | 1.419E−03 |
| PLOD3 | 2.18E−10 | −0.618 | 0.145 | 0.057 | 2.122E−06 | APITD1 | 1.48E−07 | 0.841 | 0.154 | 0.035 | 1.439E−03 |
| CDK2AP2 | 2.31E−10 | −0.456 | 0.135 | 0.057 | 2.251E−06 | NFKB1 | 1.49E−07 | 0.895 | 0.15 | 0.044 | 1.454E−03 |
| IFNAR2 | 2.34E−10 | −0.499 | 0.139 | 0.053 | 2.284E−06 | EXOC8 | 1.50E−07 | 0.637 | 0.127 | 0.026 | 1.458E−03 |
| NT5DC2 | 2.44E−10 | −0.442 | 0.121 | 0.048 | 2.377E−06 | TCEANC2 | 1.57E−07 | 0.592 | 0.103 | 0.013 | 1.531E−03 |
| CCND1 | 2.50E−10 | −0.307 | 0.198 | 0.101 | 2.431E−06 | ZNF117 | 1.57E−07 | 0.402 | 0.156 | 0.044 | 1.533E−03 |
| ARHGEF7 | 2.50E−10 | −0.323 | 0.225 | 0.127 | 2.438E−06 | NFIL3 | 1.61E−07 | 0.301 | 0.113 | 0.031 | 1.567E−03 |
| RNF138 | 2.55E−10 | −0.582 | 0.12 | 0.048 | 2.484E−06 | MTFR1 | 1.65E−07 | 0.565 | 0.145 | 0.035 | 1.604E−03 |
| MAP4K5 | 2.76E−10 | −0.610 | 0.127 | 0.057 | 2.693E−06 | IPO8 | 1.77E−07 | 0.911 | 0.124 | 0.018 | 1.723E−03 |
| ZYG11B | 2.79E−10 | −0.276 | 0.189 | 0.088 | 2.721E−06 | FCHO2 | 1.85E−07 | 0.773 | 0.109 | 0.013 | 1.805E−03 |
| NSF | 2.87E−10 | −0.664 | 0.115 | 0.053 | 2.798E−06 | CYP1B1 | 1.88E−07 | 0.561 | 0.147 | 0.044 | 1.829E−03 |
| FAM58A | 2.88E−10 | −0.407 | 0.106 | 0.031 | 2.804E−06 | TADA1 | 1.95E−07 | 1.183 | 0.101 | 0.004 | 1.905E−03 |
| TOPORS- | 2.96E−10 | −0.313 | 0.126 | 0.026 | 2.888E−06 | DERA | 1.98E−07 | 0.551 | 0.132 | 0.022 | 1.929E−03 |
| AS1 | | | | | | | | | | | |
| KCTD1 | 2.98E−10 | −0.266 | 0.139 | 0.022 | 2.899E−06 | METTL14 | 2.00E−07 | 0.293 | 0.154 | 0.044 | 1.945E−03 |
| TTF1 | 3.08E−10 | −0.659 | 0.147 | 0.092 | 3.001E−06 | PGAP2 | 2.01E−07 | 0.780 | 0.139 | 0.026 | 1.956E−03 |
| ZNF185 | 3.08E−10 | −0.281 | 0.144 | 0.035 | 3.005E−06 | TRPM7 | 2.05E−07 | 0.321 | 0.168 | 0.061 | 1.998E−03 |
| EXOSC2 | 3.18E−10 | −0.369 | 0.113 | 0.044 | 3.103E−06 | TARSL2 | 2.09E−07 | 1.273 | 0.135 | 0.022 | 2.038E−03 |
| KIF9 | 3.21E−10 | −0.411 | 0.11 | 0.031 | 3.132E−06 | BEND7 | 2.16E−07 | 0.581 | 0.12 | 0.018 | 2.102E−03 |
| EXOSC1 | 3.27E−10 | −0.459 | 0.169 | 0.075 | 3.191E−06 | EXOC4 | 2.21E−07 | 0.782 | 0.113 | 0.013 | 2.152E−03 |
| MID1IP1 | 3.31E−10 | −0.340 | 0.141 | 0.044 | 3.227E−06 | ACSL4 | 2.23E−07 | 0.353 | 0.138 | 0.035 | 2.172E−03 |
| ELP2 | 3.53E−10 | −0.628 | 0.124 | 0.057 | 3.440E−06 | THOC1 | 2.26E−07 | 0.484 | 0.148 | 0.035 | 2.201E−03 |
| STT3A | 3.54E−10 | −0.477 | 0.147 | 0.061 | 3.450E−06 | DYDC2 | 2.33E−07 | 0.771 | 0.116 | 0.013 | 2.275E−03 |
| RPUSD3 | 3.55E−10 | −0.291 | 0.156 | 0.044 | 3.460E−06 | PRKAG1 | 2.35E−07 | 0.327 | 0.138 | 0.031 | 2.294E−03 |
| GNAI1 | 3.58E−10 | −0.386 | 0.106 | 0.035 | 3.489E−06 | KMO | 2.51E−07 | 0.706 | 0.135 | 0.031 | 2.441E−03 |
| CRBN | 3.61E−10 | −0.446 | 0.175 | 0.075 | 3.513E−06 | ZFAND6 | 2.51E−07 | 0.434 | 0.138 | 0.035 | 2.445E−03 |
| C6orf1 | 3.65E−10 | −0.614 | 0.126 | 0.044 | 3.555E−06 | COG3 | 2.55E−07 | 0.366 | 0.144 | 0.044 | 2.486E−03 |
| KIF1A | 3.73E−10 | −0.311 | 0.212 | 0.118 | 3.636E−06 | LYSMD3 | 2.64E−07 | 0.602 | 0.138 | 0.031 | 2.569E−03 |
| RAB34 | 3.78E−10 | −0.571 | 0.153 | 0.096 | 3.681E−06 | METTL6 | 2.65E−07 | 0.309 | 0.115 | 0.018 | 2.582E−03 |
| METTL23 | 3.80E−10 | −0.432 | 0.166 | 0.07 | 3.702E−06 | WDR48 | 2.70E−07 | 0.939 | 0.138 | 0.026 | 2.630E−03 |
| SAP30 | 4.02E−10 | −0.289 | 0.15 | 0.039 | 3.915E−06 | OSBPL3 | 2.72E−07 | 0.389 | 0.121 | 0.022 | 2.650E−03 |
| PPIL4 | 4.05E−10 | −0.365 | 0.18 | 0.066 | 3.950E−06 | RPS8 | 2.82E−07 | 0.338 | 0.994 | 0.956 | 2.748E−03 |
| PSAT1 | 4.22E−10 | −0.731 | 0.154 | 0.088 | 4.113E−06 | DLGAP5 | 2.93E−07 | 0.385 | 0.16 | 0.07 | 2.860E−03 |
| EPC2 | 4.27E−10 | −0.763 | 0.101 | 0.044 | 4.162E−06 | KIAA0196 | 3.01E−07 | 0.712 | 0.171 | 0.057 | 2.935E−03 |
| LGMN | 4.34E−10 | −0.881 | 0.139 | 0.07 | 4.231E−06 | PARN | 3.22E−07 | 0.607 | 0.147 | 0.031 | 3.137E−03 |
| MAP2K3 | 4.34E−10 | −0.576 | 0.151 | 0.057 | 4.233E−06 | TRIM13 | 3.25E−07 | 1.055 | 0.123 | 0.026 | 3.165E−03 |
| PTRH1 | 4.41E−10 | −0.331 | 0.112 | 0.031 | 4.296E−06 | EIF2B1 | 3.32E−07 | 0.572 | 0.174 | 0.053 | 3.237E−03 |
| CCDC86 | 4.41E−10 | −0.401 | 0.126 | 0.044 | 4.302E−06 | HNRNPLL | 3.35E−07 | 0.389 | 0.135 | 0.031 | 3.260E−03 |
| MRPL43 | 4.55E−10 | −0.414 | 0.159 | 0.083 | 4.436E−06 | AIG1 | 3.64E−07 | 0.272 | 0.153 | 0.044 | 3.545E−03 |
| HERPUD2 | 4.58E−10 | −0.541 | 0.136 | 0.039 | 4.463E−06 | KIAA1033 | 3.66E−07 | 0.400 | 0.136 | 0.026 | 3.571E−03 |
| TARS2 | 4.59E−10 | −0.595 | 0.129 | 0.053 | 4.468E−06 | UBR3 | 3.67E−07 | 0.836 | 0.116 | 0.013 | 3.573E−03 |
| MUS81 | 4.74E−10 | −0.399 | 0.123 | 0.035 | 4.620E−06 | INO80D | 3.95E−07 | 0.910 | 0.106 | 0.009 | 3.848E−03 |
| DHX29 | 4.86E−10 | −0.476 | 0.165 | 0.127 | 4.732E−06 | ECHDC2 | 3.96E−07 | 0.261 | 0.118 | 0.022 | 3.857E−03 |
| ASPSCR1 | 4.88E−10 | −0.622 | 0.113 | 0.039 | 4.754E−06 | DNAAF2 | 3.98E−07 | 0.343 | 0.11 | 0.018 | 3.879E−03 |
| GIT1 | 4.88E−10 | −0.330 | 0.139 | 0.061 | 4.758E−06 | LIMK2 | 4.03E−07 | 0.265 | 0.12 | 0.026 | 3.926E−03 |
| PATL1 | 4.90E−10 | −0.527 | 0.18 | 0.083 | 4.774E−06 | IRF2 | 4.04E−07 | 0.319 | 0.147 | 0.039 | 3.934E−03 |
| NEK2 | 5.05E−10 | −0.404 | 0.221 | 0.154 | 4.917E−06 | PRRG1 | 4.19E−07 | 0.785 | 0.133 | 0.022 | 4.079E−03 |
| SHOC2 | 5.10E−10 | −0.334 | 0.231 | 0.123 | 4.973E−06 | C18orf21 | 4.21E−07 | 0.369 | 0.15 | 0.035 | 4.105E−03 |
| TAF6 | 5.52E−10 | −0.478 | 0.12 | 0.035 | 5.376E−06 | SIKE1 | 4.24E−07 | 0.571 | 0.138 | 0.026 | 4.128E−03 |
| BTBD2 | 5.58E−10 | −0.496 | 0.103 | 0.035 | 5.442E−06 | VTI1A | 4.24E−07 | 0.369 | 0.156 | 0.057 | 4.131E−03 |
| RHOD | 5.70E−10 | −0.274 | 0.133 | 0.035 | 5.553E−06 | DUSP12 | 4.42E−07 | 0.540 | 0.115 | 0.026 | 4.311E−03 |
| ASXL2 | 5.74E−10 | −0.710 | 0.166 | 0.083 | 5.589E−06 | ERC1 | 4.57E−07 | 0.377 | 0.156 | 0.053 | 4.451E−03 |
| TMEM131 | 6.40E−10 | −0.350 | 0.142 | 0.044 | 6.236E−06 | AFF1 | 4.59E−07 | 0.280 | 0.129 | 0.035 | 4.468E−03 |
| SEC22C | 6.65E−10 | −0.279 | 0.171 | 0.07 | 6.478E−06 | CDYL | 4.76E−07 | 0.561 | 0.157 | 0.07 | 4.643E−03 |
| MTERFD2 | 6.75E−10 | −0.290 | 0.141 | 0.057 | 6.581E−06 | GRIPAP1 | 4.77E−07 | 0.357 | 0.107 | 0.022 | 4.648E−03 |
| KLHL12 | 6.78E−10 | −0.645 | 0.126 | 0.053 | 6.609E−06 | FAR1 | 4.85E−07 | 0.365 | 0.171 | 0.048 | 4.729E−03 |
| CEP97 | 7.09E−10 | −0.481 | 0.115 | 0.048 | 6.914E−06 | C3orf17 | 5.05E−07 | 0.621 | 0.141 | 0.026 | 4.921E−03 |
| CEBPB | 7.59E−10 | −0.579 | 0.121 | 0.057 | 7.400E−06 | RBM33 | 5.15E−07 | 0.567 | 0.127 | 0.022 | 5.016E−03 |
| NPRL3 | 7.77E−10 | −0.701 | 0.13 | 0.083 | 7.576E−06 | UBAP1 | 5.44E−07 | 0.523 | 0.156 | 0.035 | 5.300E−03 |
| TEX261 | 7.78E−10 | −0.342 | 0.153 | 0.061 | 7.586E−06 | TTC9C | 5.50E−07 | 0.315 | 0.129 | 0.031 | 5.362E−03 |
| MLST8 | 7.89E−10 | −0.492 | 0.106 | 0.039 | 7.694E−06 | CA2 | 5.52E−07 | 0.745 | 0.123 | 0.039 | 5.382E−03 |
| TGFB2 | 8.36E−10 | −0.355 | 0.157 | 0.083 | 8.149E−06 | ABCF2 | 5.54E−07 | 1.046 | 0.127 | 0.031 | 5.394E−03 |
| ZNF766 | 8.50E−10 | −0.271 | 0.11 | 0.022 | 8.279E−06 | FNBP1L | 5.54E−07 | 0.389 | 0.141 | 0.048 | 5.402E−03 |
| GTF2A1 | 9.36E−10 | −0.310 | 0.15 | 0.057 | 9.123E−06 | GTF3C1 | 5.76E−07 | 0.751 | 0.127 | 0.026 | 5.608E−03 |
| UTRN | 9.51E−10 | −0.555 | 0.166 | 0.083 | 9.265E−06 | TUG1 | 5.81E−07 | 0.463 | 0.189 | 0.066 | 5.662E−03 |
| TOP2A | 9.61E−10 | −0.468 | 0.271 | 0.228 | 9.369E−06 | GNG4 | 5.82E−07 | 0.665 | 0.112 | 0.013 | 5.675E−03 |
| ARID1A | 1.00E−09 | −0.395 | 0.169 | 0.092 | 9.791E−06 | LATS2 | 5.89E−07 | 0.675 | 0.113 | 0.018 | 5.736E−03 |
| LPIN1 | 1.07E−09 | −0.711 | 0.168 | 0.083 | 1.045E−05 | NME6 | 5.99E−07 | 0.391 | 0.12 | 0.022 | 5.837E−03 |
| EXOSC10 | 1.09E−09 | −0.592 | 0.136 | 0.048 | 1.058E−05 | RBM28 | 6.11E−07 | 0.607 | 0.157 | 0.048 | 5.959E−03 |
| SLC35E1 | 1.09E−09 | −0.565 | 0.118 | 0.053 | 1.062E−05 | HIVEP1 | 6.27E−07 | 0.451 | 0.171 | 0.053 | 6.111E−03 |
| DCTN6 | 1.10E−09 | −0.257 | 0.135 | 0.044 | 1.074E−05 | EXOSC9 | 6.40E−07 | 1.013 | 0.113 | 0.013 | 6.241E−03 |
| TRAPPC2P1 | 1.10E−09 | −0.638 | 0.107 | 0.044 | 1.074E−05 | TTC14 | 7.06E−07 | 0.646 | 0.148 | 0.039 | 6.881E−03 |
| HMGN5 | 1.10E−09 | −0.704 | 0.123 | 0.044 | 1.075E−05 | PARVA | 7.12E−07 | 0.357 | 0.124 | 0.026 | 6.941E−03 |
| ACOT7 | 1.11E−09 | −0.390 | 0.127 | 0.053 | 1.079E−05 | GNB5 | 7.19E−07 | 0.716 | 0.112 | 0.022 | 7.005E−03 |
| FAM168B | 1.13E−09 | −0.611 | 0.18 | 0.127 | 1.099E−05 | PAPD4 | 7.39E−07 | 0.379 | 0.112 | 0.022 | 7.206E−03 |
| RAB12 | 1.16E−09 | −0.500 | 0.148 | 0.079 | 1.132E−05 | MED6 | 7.55E−07 | 0.294 | 0.145 | 0.044 | 7.354E−03 |
| CTSL | 1.28E−09 | −0.795 | 0.11 | 0.048 | 1.243E−05 | PNPLA8 | 7.70E−07 | 0.367 | 0.192 | 0.07 | 7.505E−03 |
| ZCCHC2 | 1.28E−09 | −0.351 | 0.157 | 0.066 | 1.248E−05 | REV1 | 7.77E−07 | 0.276 | 0.132 | 0.044 | 7.575E−03 |
| MAP2K2 | 1.29E−09 | −0.372 | 0.103 | 0.031 | 1.254E−05 | STYX | 7.86E−07 | 0.461 | 0.162 | 0.057 | 7.659E−03 |
| AGPAT9 | 1.30E−09 | −0.341 | 0.177 | 0.066 | 1.269E−05 | MCM9 | 8.47E−07 | 0.493 | 0.104 | 0.013 | 8.258E−03 |
| RAB5B | 1.32E−09 | −0.305 | 0.129 | 0.039 | 1.289E−05 | ARNTL | 8.53E−07 | 0.657 | 0.112 | 0.018 | 8.312E−03 |
| SGSM3 | 1.54E−09 | −0.469 | 0.127 | 0.066 | 1.498E−05 | SPRYD7 | 8.74E−07 | 0.318 | 0.116 | 0.053 | 8.513E−03 |
| PIM3 | 1.54E−09 | −0.480 | 0.127 | 0.044 | 1.503E−05 | UBQLN2 | 8.75E−07 | 0.714 | 0.142 | 0.035 | 8.528E−03 |
| MRFAP1L1 | 1.63E−09 | −0.329 | 0.156 | 0.039 | 1.592E−05 | RMI1 | 9.05E−07 | 0.499 | 0.182 | 0.061 | 8.817E−03 |
| NR2F6 | 1.64E−09 | −0.662 | 0.135 | 0.061 | 1.602E−05 | LRRK1 | 9.12E−07 | 0.476 | 0.169 | 0.061 | 8.892E−03 |
| CCSAP | 1.65E−09 | −0.498 | 0.127 | 0.061 | 1.611E−05 | QTRTD1 | 9.21E−07 | 0.464 | 0.145 | 0.044 | 8.980E−03 |
| HDHD2 | 1.67E−09 | −0.400 | 0.129 | 0.053 | 1.623E−05 | CLSPN | 9.39E−07 | 0.595 | 0.126 | 0.026 | 9.147E−03 |
| MVP | 1.70E−09 | −0.428 | 0.116 | 0.053 | 1.652E−05 | GALNT11 | 9.41E−07 | 0.511 | 0.139 | 0.035 | 9.172E−03 |
| GTPBP2 | 1.70E−09 | −0.329 | 0.115 | 0.026 | 1.653E−05 | MAML2 | 9.57E−07 | 0.333 | 0.103 | 0.018 | 9.330E−03 |
| CCNDBP1 | 1.72E−09 | −0.414 | 0.135 | 0.057 | 1.679E−05 | AGTPBP1 | 9.61E−07 | 0.575 | 0.153 | 0.039 | 9.365E−03 |
| ERCC1 | 1.92E−09 | −0.356 | 0.142 | 0.048 | 1.867E−05 | AUH | 9.62E−07 | 0.577 | 0.135 | 0.031 | 9.375E−03 |
| BPHL | 1.92E−09 | −0.493 | 0.12 | 0.026 | 1.869E−05 | ARHGEF26- | 1.01E−06 | 0.571 | 0.115 | 0.022 | 9.825E−03 |
| | | | | | | AS1 | | | | | |
| MAP3K1 | 1.93E−09 | −0.314 | 0.147 | 0.066 | 1.885E−05 | C16orf72 | 1.08E−06 | 0.565 | 0.136 | 0.039 | 1.052E−02 |
| DSTYK | 1.96E−09 | −0.308 | 0.156 | 0.048 | 1.911E−05 | DMTF1 | 1.09E−06 | 1.007 | 0.104 | 0.009 | 1.059E−02 |
| CABIN1 | 1.99E−09 | −0.322 | 0.104 | 0.026 | 1.939E−05 | USP33 | 1.11E−06 | 0.347 | 0.171 | 0.061 | 1.086E−02 |
| MAPK3 | 2.03E−09 | −0.573 | 0.123 | 0.061 | 1.976E−05 | ZNF92 | 1.12E−06 | 0.370 | 0.11 | 0.022 | 1.088E−02 |
| MTPAP | 2.16E−09 | −0.433 | 0.121 | 0.031 | 2.109E−05 | DENND1A | 1.14E−06 | 0.659 | 0.109 | 0.018 | 1.106E−02 |
| CHURC1 | 2.22E−09 | −0.389 | 0.165 | 0.07 | 2.162E−05 | GABPA | 1.15E−06 | 0.463 | 0.118 | 0.022 | 1.118E−02 |
| SAMHD1 | 2.22E−09 | −0.315 | 0.183 | 0.083 | 2.164E−05 | DSN1 | 1.17E−06 | 0.655 | 0.113 | 0.022 | 1.141E−02 |
| CADM1 | 2.39E−09 | −0.510 | 0.139 | 0.053 | 2.331E−05 | AC119673.1 | 1.18E−06 | 0.558 | 0.133 | 0.031 | 1.150E−02 |
| NUDT22 | 2.44E−09 | −0.409 | 0.118 | 0.031 | 2.381E−05 | RPGRIP1L | 1.19E−06 | 0.392 | 0.112 | 0.018 | 1.160E−02 |
| TPP1 | 2.49E−09 | −0.383 | 0.13 | 0.061 | 2.427E−05 | KDM4C | 1.22E−06 | 0.615 | 0.123 | 0.035 | 1.187E−02 |
| FLII | 2.56E−09 | −0.441 | 0.168 | 0.088 | 2.494E−05 | LXN | 1.22E−06 | 0.415 | 0.104 | 0.026 | 1.189E−02 |
| GDAP2 | 2.57E−09 | −0.652 | 0.107 | 0.057 | 2.506E−05 | ADAT1 | 1.24E−06 | 0.287 | 0.11 | 0.018 | 1.208E−02 |
| MYD88 | 2.64E−09 | −0.261 | 0.132 | 0.039 | 2.571E−05 | PACS1 | 1.25E−06 | 0.695 | 0.106 | 0.044 | 1.220E−02 |
| CCM2 | 2.76E−09 | −0.448 | 0.133 | 0.053 | 2.686E−05 | ARRDC4 | 1.26E−06 | 0.319 | 0.142 | 0.039 | 1.232E−02 |
| TMC6 | 2.87E−09 | −0.529 | 0.139 | 0.066 | 2.796E−05 | UTP14C | 1.30E−06 | 0.332 | 0.135 | 0.035 | 1.269E−02 |
| EHBP1 | 2.94E−09 | −0.486 | 0.129 | 0.057 | 2.864E−05 | ETS2 | 1.31E−06 | 0.262 | 0.11 | 0.026 | 1.281E−02 |
| HTRA2 | 3.14E−09 | −0.286 | 0.135 | 0.048 | 3.061E−05 | TIPARP | 1.39E−06 | 0.839 | 0.162 | 0.048 | 1.355E−02 |
| SS18 | 3.48E−09 | −0.570 | 0.12 | 0.057 | 3.395E−05 | MIF4GD | 1.48E−06 | 0.407 | 0.106 | 0.018 | 1.439E−02 |
| MUM1 | 3.94E−09 | −0.265 | 0.104 | 0.018 | 3.843E−05 | CYP20A1 | 1.48E−06 | 0.564 | 0.116 | 0.022 | 1.439E−02 |
| MAD2L1 | 4.11E−09 | −0.351 | 0.2 | 0.11 | 4.001E−05 | KLHL42 | 1.49E−06 | 0.699 | 0.106 | 0.018 | 1.451E−02 |
| LONP1 | 4.17E−09 | −0.339 | 0.115 | 0.039 | 4.065E−05 | CYLD | 1.58E−06 | 0.357 | 0.168 | 0.057 | 1.538E−02 |
| NAGA | 4.30E−09 | −0.389 | 0.124 | 0.039 | 4.187E−05 | COX18 | 1.64E−06 | 0.252 | 0.127 | 0.044 | 1.597E−02 |
| VPS13C | 4.31E−09 | −0.591 | 0.165 | 0.083 | 4.196E−05 | PKIG | 1.68E−06 | 0.340 | 0.104 | 0.022 | 1.636E−02 |
| RAB3D | 4.51E−09 | −0.359 | 0.121 | 0.031 | 4.394E−05 | PCCA | 1.68E−06 | 0.264 | 0.13 | 0.048 | 1.639E−02 |
| TRIAP1 | 4.83E−09 | −0.730 | 0.115 | 0.053 | 4.709E−05 | PPM1F | 1.71E−06 | 0.292 | 0.101 | 0.022 | 1.666E−02 |
| TMEM19 | 5.13E−09 | −0.568 | 0.135 | 0.061 | 5.002E−05 | GGA2 | 1.73E−06 | 0.389 | 0.15 | 0.044 | 1.685E−02 |
| FAM217B | 5.58E−09 | −0.391 | 0.121 | 0.039 | 5.437E−05 | S100PBP | 1.77E−06 | 0.402 | 0.141 | 0.048 | 1.723E−02 |
| GNE | 5.75E−09 | −0.525 | 0.101 | 0.031 | 5.604E−05 | CAAP1 | 1.80E−06 | 0.379 | 0.166 | 0.053 | 1.751E−02 |
| STPG1 | 6.36E−09 | −0.347 | 0.121 | 0.053 | 6.196E−05 | PPIL6 | 1.84E−06 | 0.598 | 0.101 | 0.013 | 1.794E−02 |
| CDK10 | 6.41E−09 | −0.395 | 0.106 | 0.048 | 6.249E−05 | DNTTIP1 | 1.85E−06 | 0.513 | 0.147 | 0.048 | 1.800E−02 |
| GLIPR2 | 6.48E−09 | −0.584 | 0.139 | 0.061 | 6.317E−05 | ST6GAL1 | 1.98E−06 | 0.953 | 0.124 | 0.022 | 1.934E−02 |
| SART1 | 6.49E−09 | −0.341 | 0.109 | 0.035 | 6.328E−05 | SOCS4 | 2.03E−06 | 0.312 | 0.153 | 0.048 | 1.979E−02 |
| ATF5 | 6.62E−09 | −0.368 | 0.129 | 0.044 | 6.452E−05 | STK39 | 2.05E−06 | 0.259 | 0.112 | 0.022 | 2.002E−02 |
| TMEM41A | 6.70E−09 | −0.513 | 0.138 | 0.061 | 6.531E−05 | KIF2A | 2.19E−06 | 0.447 | 0.165 | 0.075 | 2.137E−02 |
| PNPLA4 | 6.97E−09 | −0.280 | 0.124 | 0.035 | 6.795E−05 | ZNF678 | 2.24E−06 | 0.422 | 0.115 | 0.039 | 2.180E−02 |
| SPOPL | 7.54E−09 | −0.516 | 0.104 | 0.053 | 7.344E−05 | DMXL1 | 2.30E−06 | 0.300 | 0.118 | 0.031 | 2.244E−02 |
| NDST1 | 7.68E−09 | −0.253 | 0.116 | 0.026 | 7.485E−05 | SNAPC3 | 2.35E−06 | 0.614 | 0.151 | 0.048 | 2.289E−02 |
| SUN2 | 7.98E−09 | −0.450 | 0.169 | 0.083 | 7.774E−05 | DPH7 | 2.38E−06 | 0.649 | 0.11 | 0.018 | 2.317E−02 |
| BOD1L1 | 8.52E−09 | −0.503 | 0.135 | 0.048 | 8.303E−05 | FKBP15 | 2.38E−06 | 0.259 | 0.124 | 0.031 | 2.322E−02 |
| TSC1 | 8.76E−09 | −0.290 | 0.142 | 0.061 | 8.534E−05 | ALG8 | 2.39E−06 | 0.373 | 0.133 | 0.039 | 2.330E−02 |
| OXCT1 | 9.21E−09 | −0.597 | 0.107 | 0.057 | 8.979E−05 | CNTRL | 2.42E−06 | 0.660 | 0.124 | 0.022 | 2.357E−02 |
| NCK2 | 9.37E−09 | −0.336 | 0.138 | 0.061 | 9.131E−05 | APOOL | 2.47E−06 | 1.412 | 0.121 | 0.026 | 2.408E−02 |
| GBA | 9.44E−09 | −0.573 | 0.101 | 0.057 | 9.200E−05 | SH3BP5 | 2.53E−06 | 0.425 | 0.12 | 0.026 | 2.469E−02 |
| AGPS | 9.61E−09 | −0.643 | 0.11 | 0.031 | 9.366E−05 | IQCG | 2.87E−06 | 0.518 | 0.101 | 0.022 | 2.798E−02 |
| ZNF326 | 9.63E−09 | −0.454 | 0.168 | 0.061 | 9.382E−05 | TRAPPC8 | 2.92E−06 | 0.472 | 0.127 | 0.035 | 2.841E−02 |
| C7orf55 | 9.72E−09 | −0.395 | 0.124 | 0.044 | 9.473E−05 | RIN2 | 3.02E−06 | 0.424 | 0.116 | 0.026 | 2.942E−02 |
| C1orf63 | 1.00E−08 | −0.310 | 0.191 | 0.096 | 9.777E−05 | SLC20A1 | 3.12E−06 | 0.324 | 0.113 | 0.044 | 3.043E−02 |
| WSB2 | 1.05E−08 | −0.384 | 0.139 | 0.053 | 1.021E−04 | C6orf211 | 3.14E−06 | 0.370 | 0.104 | 0.018 | 3.055E−02 |
| ZNF608 | 1.07E−08 | −0.267 | 0.123 | 0.044 | 1.040E−04 | SH3D19 | 3.25E−06 | 0.282 | 0.11 | 0.026 | 3.164E−02 |
| HNRNPA1L2 | 1.09E−08 | −0.430 | 0.113 | 0.039 | 1.064E−04 | TDP2 | 3.30E−06 | 0.751 | 0.127 | 0.044 | 3.217E−02 |
| HAGH | 1.11E−08 | −0.306 | 0.113 | 0.044 | 1.079E−04 | HRASLS | 3.33E−06 | 0.494 | 0.112 | 0.022 | 3.242E−02 |
| ORMDL3 | 1.19E−08 | −0.848 | 0.103 | 0.061 | 1.157E−04 | EPS8 | 3.37E−06 | 0.291 | 0.13 | 0.044 | 3.287E−02 |
| DMD | 1.19E−08 | −0.287 | 0.188 | 0.092 | 1.164E−04 | ABCD3 | 3.59E−06 | 0.317 | 0.106 | 0.026 | 3.501E−02 |
| RHBDD2 | 1.31E−08 | −0.678 | 0.103 | 0.053 | 1.273E−04 | METTL3 | 3.64E−06 | 0.346 | 0.107 | 0.026 | 3.543E−02 |
| RP11- | 1.34E−08 | −0.635 | 0.123 | 0.061 | 1.306E−04 | MBTD1 | 3.71E−06 | 0.535 | 0.126 | 0.035 | 3.615E−02 |
| 792A8.4 | | | | | | | | | | | |
| DUSP11 | 1.34E−08 | −0.307 | 0.12 | 0.044 | 1.307E−04 | SHQ1 | 3.79E−06 | 0.532 | 0.129 | 0.039 | 3.691E−02 |
| LRRC47 | 1.42E−08 | −0.363 | 0.113 | 0.039 | 1.383E−04 | KLHDC10 | 3.79E−06 | 0.292 | 0.112 | 0.035 | 3.697E−02 |
| ADPRHL2 | 1.42E−08 | −0.644 | 0.115 | 0.053 | 1.383E−04 | CPVL | 3.79E−06 | 0.710 | 0.132 | 0.035 | 3.698E−02 |
| PSMD5-AS1 | 1.43E−08 | −0.334 | 0.135 | 0.048 | 1.393E−04 | ZNF429 | 3.81E−06 | 0.273 | 0.107 | 0.026 | 3.709E−02 |
| RORA | 1.55E−08 | −0.293 | 0.118 | 0.053 | 1.511E−04 | POLR3F | 3.81E−06 | 0.527 | 0.106 | 0.018 | 3.712E−02 |
| NOC3L | 1.60E−08 | −0.410 | 0.174 | 0.083 | 1.560E−04 | BCL10 | 3.86E−06 | 0.431 | 0.142 | 0.044 | 3.763E−02 |
| TULP4 | 1.60E−08 | −0.496 | 0.154 | 0.083 | 1.561E−04 | RIOK2 | 3.87E−06 | 0.690 | 0.107 | 0.018 | 3.771E−02 |
| CSGALNACT2 | 1.63E−08 | −0.576 | 0.126 | 0.048 | 1.593E−04 | TRAFD1 | 4.07E−06 | 0.645 | 0.106 | 0.018 | 3.964E−02 |
| ARHGAP1 | 1.67E−08 | −0.465 | 0.121 | 0.048 | 1.625E−04 | EFNB2 | 4.22E−06 | 0.262 | 0.112 | 0.026 | 4.116E−02 |
| TRAPPC4 | 1.68E−08 | −0.253 | 0.13 | 0.053 | 1.641E−04 | SF3A1 | 4.28E−06 | 0.478 | 0.116 | 0.031 | 4.171E−02 |
| TACC3 | 1.71E−08 | −0.413 | 0.126 | 0.048 | 1.668E−04 | WDR70 | 4.87E−06 | 0.539 | 0.104 | 0.018 | 4.750E−02 |
| KIF20B | 1.83E−08 | −0.635 | 0.148 | 0.11 | 1.788E−04 | ZNF7 | 4.89E−06 | 0.983 | 0.126 | 0.026 | 4.761E−02 |
| ITGAV | 1.86E−08 | −0.322 | 0.129 | 0.053 | 1.813E−04 | MBIP | 4.91E−06 | 0.322 | 0.123 | 0.039 | 4.782E−02 |
| CHD3 | 1.88E−08 | −0.345 | 0.141 | 0.061 | 1.829E−04 | RALGAPA2 | 5.00E−06 | 0.450 | 0.104 | 0.031 | 4.871E−02 |
| HMGXB3 | 1.89E−08 | −0.667 | 0.109 | 0.061 | 1.838E−04 | | | | | | |
| PPP1R3B | 2.04E−08 | −0.639 | 0.112 | 0.061 | 1.986E−04 | | | | | | |
| MON1B | 2.19E−08 | −0.417 | 0.12 | 0.044 | 2.132E−04 | | | | | | |
| DUSP16 | 2.33E−08 | −0.289 | 0.112 | 0.031 | 2.268E−04 | | | | | | |
| HELLS | 2.35E−08 | −0.415 | 0.175 | 0.101 | 2.295E−04 | | | | | | |
| STK17B | 2.40E−08 | −0.746 | 0.115 | 0.079 | 2.340E−04 | | | | | | |
| CPSF7 | 2.45E−08 | −0.441 | 0.116 | 0.066 | 2.392E−04 | | | | | | |
| NMT2 | 2.76E−08 | −0.433 | 0.139 | 0.057 | 2.687E−04 | | | | | | |
| CEPT1 | 2.88E−08 | −0.275 | 0.124 | 0.039 | 2.804E−04 | | | | | | |
| SDHAF2 | 2.97E−08 | −0.448 | 0.106 | 0.035 | 2.892E−04 | | | | | | |
| PRPF8 | 3.06E−08 | −0.496 | 0.148 | 0.075 | 2.984E−04 | | | | | | |
| GNS | 3.09E−08 | −0.385 | 0.156 | 0.079 | 3.012E−04 | | | | | | |
| HOOK3 | 3.29E−08 | −0.320 | 0.136 | 0.053 | 3.204E−04 | | | | | | |
| HIRIP3 | 3.30E−08 | −0.459 | 0.107 | 0.031 | 3.216E−04 | | | | | | |
| LYRM5 | 3.50E−08 | −0.276 | 0.121 | 0.022 | 3.407E−04 | | | | | | |
| FOXN2 | 3.53E−08 | −0.258 | 0.159 | 0.057 | 3.440E−04 | | | | | | |
| PELP1 | 3.64E−08 | −0.310 | 0.112 | 0.035 | 3.543E−04 | | | | | | |
| SGOL2 | 3.71E−08 | −0.656 | 0.147 | 0.061 | 3.612E−04 | | | | | | |
| GPR183 | 3.84E−08 | −0.497 | 0.195 | 0.127 | 3.745E−04 | | | | | | |
| PKN2 | 3.91E−08 | −0.403 | 0.147 | 0.07 | 3.808E−04 | | | | | | |
| CCDC92 | 4.18E−08 | −0.515 | 0.106 | 0.022 | 4.071E−04 | | | | | | |
| TSPAN15 | 4.68E−08 | −0.305 | 0.11 | 0.035 | 4.556E−04 | | | | | | |
| ANAPC15 | 5.04E−08 | −0.315 | 0.13 | 0.066 | 4.911E−04 | | | | | | |
| CMTM7 | 5.09E−08 | −0.455 | 0.132 | 0.079 | 4.959E−04 | | | | | | |
| CDK7 | 5.11E−08 | −0.413 | 0.101 | 0.026 | 4.983E−04 | | | | | | |
| RB1CC1 | 5.12E−08 | −0.252 | 0.204 | 0.105 | 4.991E−04 | | | | | | |
| SP4 | 5.16E−08 | −0.527 | 0.112 | 0.039 | 5.025E−04 | | | | | | |
| MTMR1 | 5.36E−08 | −0.347 | 0.115 | 0.035 | 5.222E−04 | | | | | | |
| AUTS2 | 6.14E−08 | −0.251 | 0.153 | 0.083 | 5.988E−04 | | | | | | |
| ALAS1 | 7.06E−08 | −0.505 | 0.168 | 0.114 | 6.879E−04 | | | | | | |
| SERPINB6 | 7.22E−08 | −0.283 | 0.106 | 0.039 | 7.036E−04 | | | | | | |
| KIN | 7.94E−08 | −0.560 | 0.15 | 0.079 | 7.735E−04 | | | | | | |
| UBTD2 | 8.22E−08 | −0.352 | 0.126 | 0.057 | 8.009E−04 | | | | | | |
| PWP2 | 8.31E−08 | −0.493 | 0.109 | 0.039 | 8.097E−04 | | | | | | |
| SPA17 | 8.33E−08 | −0.551 | 0.121 | 0.057 | 8.121E−04 | | | | | | |
| KLHL9 | 9.12E−08 | −0.327 | 0.142 | 0.048 | 8.889E−04 | | | | | | |
| FBXO34 | 9.34E−08 | −0.291 | 0.168 | 0.083 | 9.104E−04 | | | | | | |
| MRPS9 | 9.89E−08 | −0.262 | 0.132 | 0.048 | 9.634E−04 | | | | | | |
| IFIT2 | 9.94E−08 | −0.431 | 0.15 | 0.083 | 9.682E−04 | | | | | | |
| VAMP4 | 1.00E−07 | −0.571 | 0.116 | 0.061 | 9.767E−04 | | | | | | |
| PYGB | 1.03E−07 | −0.577 | 0.109 | 0.035 | 1.003E−03 | | | | | | |
| DLAT | 1.07E−07 | −0.340 | 0.13 | 0.053 | 1.043E−03 | | | | | | |
| NEAT1 | 1.08E−07 | −1.230 | 0.253 | 0.154 | 1.048E−03 | | | | | | |
| DYRK1A | 1.12E−07 | −0.264 | 0.138 | 0.053 | 1.091E−03 | | | | | | |
| GAN | 1.17E−07 | −0.370 | 0.107 | 0.061 | 1.141E−03 | | | | | | |
| SUPT6H | 1.19E−07 | −0.527 | 0.107 | 0.048 | 1.163E−03 | | | | | | |
| ZNF770 | 1.23E−07 | −0.331 | 0.157 | 0.083 | 1.195E−03 | | | | | | |
| ENG | 1.35E−07 | −0.307 | 0.116 | 0.057 | 1.319E−03 | | | | | | |
| NUMA1 | 1.43E−07 | −0.260 | 0.153 | 0.075 | 1.391E−03 | | | | | | |
| TAF3 | 1.43E−07 | −0.256 | 0.123 | 0.048 | 1.398E−03 | | | | | | |
| APC | 1.93E−07 | −0.641 | 0.127 | 0.083 | 1.881E−03 | | | | | | |
| RBMXL1 | 2.02E−07 | −0.340 | 0.115 | 0.035 | 1.966E−03 | | | | | | |
| RGMA | 2.10E−07 | −0.318 | 0.126 | 0.044 | 2.049E−03 | | | | | | |
| EPSTI1 | 2.12E−07 | −0.313 | 0.148 | 0.079 | 2.068E−03 | | | | | | |
| CASC7 | 2.21E−07 | −0.779 | 0.13 | 0.061 | 2.151E−03 | | | | | | |
| PTPRA | 2.28E−07 | −0.377 | 0.127 | 0.048 | 2.223E−03 | | | | | | |
| TIGD2 | 2.32E−07 | −0.548 | 0.101 | 0.053 | 2.265E−03 | | | | | | |
| ZC3H8 | 2.34E−07 | −0.262 | 0.124 | 0.048 | 2.285E−03 | | | | | | |
| RFX5 | 2.38E−07 | −0.285 | 0.116 | 0.048 | 2.319E−03 | | | | | | |
| GCN1L1 | 2.48E−07 | −0.406 | 0.101 | 0.039 | 2.418E−03 | | | | | | |
| MGAT4A | 2.62E−07 | −0.274 | 0.11 | 0.022 | 2.555E−03 | | | | | | |
| STARD3 | 2.73E−07 | −0.421 | 0.11 | 0.039 | 2.658E−03 | | | | | | |
| CENPC | 2.86E−07 | −0.553 | 0.127 | 0.057 | 2.785E−03 | | | | | | |
| CHCHD7 | 3.40E−07 | −0.368 | 0.124 | 0.057 | 3.312E−03 | | | | | | |
| KLHDC4 | 3.59E−07 | −0.429 | 0.107 | 0.053 | 3.503E−03 | | | | | | |
| ABL2 | 4.00E−07 | −0.493 | 0.12 | 0.061 | 3.900E−03 | | | | | | |
| IQGAP2 | 4.17E−07 | −0.498 | 0.145 | 0.092 | 4.068E−03 | | | | | | |
| BMPR1A | 4.22E−07 | −0.572 | 0.118 | 0.066 | 4.116E−03 | | | | | | |
| XPC | 4.48E−07 | −0.634 | 0.106 | 0.048 | 4.366E−03 | | | | | | |
| TEAD1 | 4.69E−07 | −0.289 | 0.136 | 0.061 | 4.572E−03 | | | | | | |
| MTA2 | 4.76E−07 | −0.312 | 0.123 | 0.057 | 4.636E−03 | | | | | | |
| HMGN4 | 4.88E−07 | −0.253 | 0.107 | 0.044 | 4.751E−03 | | | | | | |
| MED23 | 6.12E−07 | −0.461 | 0.107 | 0.053 | 5.959E−03 | | | | | | |
| G2E3 | 6.16E−07 | −0.493 | 0.13 | 0.053 | 6.003E−03 | | | | | | |
| MORC2 | 6.20E−07 | −0.301 | 0.107 | 0.035 | 6.045E−03 | | | | | | |
| SIRT7 | 6.25E−07 | −0.255 | 0.148 | 0.057 | 6.086E−03 | | | | | | |
| AGO1 | 6.91E−07 | −0.798 | 0.112 | 0.053 | 6.735E−03 | | | | | | |
| BRWD3 | 7.72E−07 | −0.280 | 0.103 | 0.044 | 7.528E−03 | | | | | | |
| MAST2 | 7.91E−07 | −0.345 | 0.118 | 0.053 | 7.704E−03 | | | | | | |
| SACM1L | 7.95E−07 | −0.291 | 0.13 | 0.053 | 7.748E−03 | | | | | | |
| MRPS27 | 8.53E−07 | −0.338 | 0.101 | 0.039 | 8.312E−03 | | | | | | |
| LIG1 | 8.60E−07 | −0.505 | 0.104 | 0.039 | 8.382E−03 | | | | | | |
| SLC39A3 | 9.89E−07 | −0.304 | 0.107 | 0.048 | 9.641E−03 | | | | | | |
| TRMT10A | 1.05E−06 | −0.284 | 0.127 | 0.066 | 1.026E−02 | | | | | | |
| GLE1 | 1.12E−06 | −0.400 | 0.113 | 0.061 | 1.089E−02 | | | | | | |
| COMMD5 | 1.27E−06 | −0.519 | 0.109 | 0.039 | 1.239E−02 | | | | | | |
| HELZ2 | 1.47E−06 | −0.497 | 0.104 | 0.044 | 1.433E−02 | | | | | | |
| RAB32 | 1.51E−06 | −0.831 | 0.104 | 0.057 | 1.475E−02 | | | | | | |
| LOX | 1.60E−06 | −0.514 | 0.124 | 0.079 | 1.558E−02 | | | | | | |
| MKNK2 | 1.76E−06 | −0.352 | 0.113 | 0.061 | 1.716E−02 | | | | | | |
| TAF13 | 1.84E−06 | −0.317 | 0.118 | 0.044 | 1.795E−02 | | | | | | |
| SIGIRR | 1.89E−06 | −0.473 | 0.107 | 0.07 | 1.842E−02 | | | | | | |
| ZNF302 | 1.96E−06 | −0.591 | 0.124 | 0.057 | 1.909E−02 | | | | | | |
| C1orf123 | 2.05E−06 | −0.419 | 0.118 | 0.048 | 2.000E−02 | | | | | | |
| TECR | 2.15E−06 | −0.589 | 0.112 | 0.061 | 2.098E−02 | | | | | | |
| SETD4 | 2.21E−06 | −0.315 | 0.103 | 0.035 | 2.156E−02 | | | | | | |
| RBI | 2.26E−06 | −0.519 | 0.115 | 0.079 | 2.201E−02 | | | | | | |
| RAF1 | 2.42E−06 | −0.341 | 0.121 | 0.053 | 2.357E−02 | | | | | | |
| CNOT4 | 2.61E−06 | −0.483 | 0.126 | 0.048 | 2.539E−02 | | | | | | |
| RPF1 | 2.97E−06 | −0.357 | 0.11 | 0.039 | 2.897E−02 | | | | | | |
| PGM2 | 3.12E−06 | −0.314 | 0.112 | 0.044 | 3.040E−02 | | | | | | |
| MRPS26 | 3.12E−06 | −0.313 | 0.113 | 0.048 | 3.040E−02 | | | | | | |
| CCDC82 | 3.46E−06 | −0.495 | 0.127 | 0.075 | 3.369E−02 | | | | | | |
| C4orf33 | 3.55E−06 | −0.302 | 0.135 | 0.057 | 3.461E−02 | | | | | | |
| RNF14 | 3.86E−06 | −0.310 | 0.113 | 0.066 | 3.763E−02 | | | | | | |

| | |
|---|---|
| CSF.patient.037.TvUT.tumor.bimod.markers.dn.1 | CSF.patient.037.TvUT.tumor.bimod.markers.up.1 |

| | p_val | avg_logFC | pct.1 | pct.2 | p_val_adj | | p_val | avg_logFC | pct.1 | pct.2 | p_val_adj |
|---|---|---|---|---|---|---|---|---|---|---|---|
| RPL32 | 0 | −2.397 | 0.759 | 0.994 | 0.000E+00 | TMSB10 | 1.14E−280 | 1.951 | 0.999 | 0.938 | 1.315E−276 |
| RPS24 | 0 | −2.520 | 0.879 | 1 | 0.000E+00 | RARRES1 | 7.06E−280 | 3.721 | 0.965 | 0.02 | 8.143E−276 |
| RPL19 | 0 | −5.110 | 0.774 | 1 | 0.000E+00 | MT-CO3 | 1.93E−277 | 1.891 | 0.998 | 0.862 | 2.225E−273 |
| SCGB2A2 | 0 | −5.451 | 0.011 | 1 | 0.000E+00 | IFI27 | 6.14E−263 | 3.288 | 0.958 | 0.056 | 7.085E−259 |
| DCD | 9.86E−279 | −4.529 | 0 | 0.932 | 1.138E−274 | B2M | 6.74E−222 | 1.690 | 0.991 | 0.882 | 7.780E−218 |
| SCGB1D2 | 4.69E−261 | −2.712 | 0.002 | 0.907 | 5.414E−257 | MFGE8 | 5.82E−202 | 2.590 | 0.857 | 0.003 | 6.720E−198 |
| MIEN1 | 2.51E−249 | −2.588 | 0.115 | 0.952 | 2.892E−245 | GLYATL2 | 3.49E−170 | 2.275 | 0.798 | 0.037 | 4.026E−166 |
| ERBB2 | 1.13E−233 | −2.271 | 0.024 | 0.896 | 1.307E−229 | PIGR | 1.02E−169 | 2.423 | 0.784 | 0.006 | 1.181E−165 |
| RPL8 | 1.18E−206 | −1.590 | 0.648 | 0.983 | 1.365E−202 | MT-CO1 | 3.84E−169 | 1.854 | 0.99 | 0.89 | 4.430E−165 |
| RPS14 | 3.17E−205 | −1.735 | 0.815 | 0.997 | 3.653E−201 | SLPI | 6.90E−169 | 2.219 | 0.861 | 0.234 | 7.961E−165 |
| RPS19 | 5.56E−204 | −1.798 | 0.673 | 0.992 | 6.418E−200 | S100A6 | 3.38E−163 | 1.909 | 0.896 | 0.53 | 3.903E−159 |
| RPS28 | 6.23E−202 | −1.901 | 0.117 | 0.924 | 7.186E−198 | MT-RNR2 | 1.75E−155 | 1.532 | 1 | 1 | 2.014E−151 |
| FOS | 7.64E−201 | −1.866 | 0.035 | 0.856 | 8.813E−197 | ARPC5 | 2.66E−142 | 1.873 | 0.827 | 0.355 | 3.070E−138 |
| RPL41 | 3.88E−200 | −1.844 | 0.47 | 0.966 | 4.474E−196 | CD59 | 7.71E−139 | 1.756 | 0.802 | 0.411 | 8.897E−135 |
| IER2 | 2.70E−199 | −2.029 | 0.031 | 0.842 | 3.114E−195 | YBX1 | 4.65E−135 | 1.633 | 0.811 | 0.482 | 5.364E−131 |
| RPS21 | 8.38E−190 | −1.660 | 0.927 | 0.997 | 9.666E−186 | CD47 | 1.68E−128 | 1.759 | 0.674 | 0.127 | 1.935E−124 |
| JUNB | 5.41E−189 | −1.955 | 0.039 | 0.828 | 6.245E−185 | TPM3 | 1.08E−117 | 1.380 | 0.744 | 0.527 | 1.247E−113 |
| PNMT | 3.42E−178 | −1.282 | 0 | 0.699 | 3.943E−174 | HEBP2 | 1.44E−112 | 1.480 | 0.694 | 0.363 | 1.659E−108 |
| EGR1 | 6.84E−177 | −1.710 | 0.01 | 0.73 | 7.898E−173 | GUK1 | 3.20E−110 | 1.317 | 0.686 | 0.437 | 3.690E−106 |
| RPL26 | 4.32E−168 | −1.729 | 0.368 | 0.938 | 4.980E−164 | LCN2 | 3.08E−108 | 1.692 | 0.596 | 0.014 | 3.549E−104 |
| PLP1 | 1.31E−166 | −1.571 | 0.002 | 0.67 | 1.507E−162 | EPCAM | 5.74E−108 | 1.362 | 0.669 | 0.445 | 6.627E−104 |
| CTTN | 7.70E−164 | −1.471 | 0.077 | 0.848 | 8.882E−160 | MT-ND4L | 2.30E−107 | 1.591 | 0.844 | 0.611 | 2.652E−103 |
| MED1 | 3.28E−162 | −1.474 | 0.064 | 0.82 | 3.791E−158 | NDUFS5 | 9.43E−105 | 1.307 | 0.653 | 0.414 | 1.089E−100 |
| RPS18 | 6.60E−162 | −1.637 | 0.905 | 0.975 | 7.614E−158 | NUCKS1 | 2.22E−104 | 1.330 | 0.87 | 0.727 | 2.562E−100 |
| CAMK2N1 | 4.01E−158 | −1.087 | 0.001 | 0.648 | 4.623E−154 | CACYBP | 2.30E−104 | 1.244 | 0.608 | 0.377 | 2.654E−100 |
| SNHG8 | 7.76E−157 | −1.668 | 0.206 | 0.904 | 8.956E−153 | NDUFS6 | 1.44E−103 | 1.158 | 0.633 | 0.448 | 1.661E−99 |
| CYP4Z1 | 1.74E−156 | −1.208 | 0 | 0.634 | 2.011E−152 | CCT5 | 1.93E−102 | 1.133 | 0.642 | 0.51 | 2.228E−98 |
| HSPA1A | 2.27E−156 | −1.656 | 0.078 | 0.806 | 2.623E−152 | IFI6 | 2.36E−101 | 1.552 | 0.749 | 0.285 | 2.725E−97 |
| ADIRF | 2.66E−156 | −1.225 | 0.015 | 0.699 | 3.069E−152 | PRDX1 | 1.69E−99 | 1.172 | 0.787 | 0.693 | 1.946E−95 |
| CCND1 | 1.78E−153 | −1.594 | 0.077 | 0.808 | 2.051E−149 | TPM4 | 4.28E−99 | 1.179 | 0.751 | 0.597 | 4.943E−95 |
| AGR2 | 2.71E−153 | −1.212 | 0.005 | 0.654 | 3.130E−149 | TM9SF2 | 6.45E−99 | 1.119 | 0.6 | 0.456 | 7.449E−95 |
| RPL39 | 3.92E−153 | −1.409 | 0.556 | 0.972 | 4.521E−149 | CAPN2 | 3.01E−98 | 1.368 | 0.629 | 0.285 | 3.470E−94 |
| MT-TP | 1.53E−151 | −2.014 | 0.023 | 0.69 | 1.764E−147 | YWHAZ | 9.79E−96 | 1.128 | 0.768 | 0.642 | 1.130E−91 |
| LRRC26 | 7.78E−151 | −0.968 | 0.001 | 0.625 | 8.984E−147 | MTRNR2L8 | 1.72E−95 | 1.522 | 0.731 | 0.454 | 1.985E−91 |
| RAB11FIP1 | 2.97E−150 | −1.757 | 0.13 | 0.856 | 3.423E−146 | SAT1 | 8.84E−95 | 1.330 | 0.711 | 0.448 | 1.021E−90 |
| XBP1 | 4.15E−149 | −1.553 | 0.155 | 0.882 | 4.794E−145 | MYL6 | 9.03E−95 | 1.178 | 0.821 | 0.685 | 1.042E−90 |
| STARD3 | 9.28E−149 | −0.974 | 0.023 | 0.696 | 1.072E−144 | MGST3 | 2.76E−92 | 1.141 | 0.531 | 0.276 | 3.180E−88 |
| S100A8 | 6.87E−147 | −2.067 | 0.023 | 0.685 | 7.929E−143 | MGP | 1.31E−91 | 1.270 | 0.892 | 0.639 | 1.507E−87 |
| RPLP0 | 1.78E−146 | −1.365 | 0.797 | 0.983 | 2.053E−142 | NDUFA4 | 1.03E−90 | 1.195 | 0.791 | 0.685 | 1.186E−86 |
| HES1 | 9.35E−145 | −1.802 | 0.129 | 0.837 | 1.079E−140 | HSD17B12 | 3.63E−90 | 1.111 | 0.49 | 0.335 | 4.195E−86 |
| MT-TV | 2.01E−143 | −1.499 | 0.04 | 0.699 | 2.325E−139 | COX4I1 | 1.62E−89 | 0.998 | 0.768 | 0.704 | 1.864E−85 |
| GRB7 | 5.28E−143 | −0.961 | 0.013 | 0.651 | 6.096E−139 | TM4SF1 | 3.09E−88 | 1.177 | 0.764 | 0.383 | 3.566E−84 |
| PGAP3 | 1.10E−140 | −0.803 | 0.001 | 0.592 | 1.269E−136 | FXYD3 | 6.57E−88 | 1.264 | 0.589 | 0.352 | 7.582E−84 |
| VDAC2 | 9.70E−140 | −1.264 | 0.079 | 0.789 | 1.119E−135 | ENAH | 2.67E−87 | 1.333 | 0.578 | 0.276 | 3.081E−83 |
| NDUFC2 | 1.18E−136 | −1.427 | 0.139 | 0.845 | 1.367E−132 | MT-CO2 | 6.27E−86 | 1.020 | 0.918 | 0.775 | 7.236E−82 |
| LDHA | 3.42E−135 | −1.533 | 0.628 | 0.966 | 3.951E−131 | TMSB4X | 3.25E−85 | 1.233 | 0.79 | 0.507 | 3.747E−81 |
| NUPR1 | 1.08E−134 | −1.545 | 0.115 | 0.817 | 1.249E−130 | CP | 2.41E−84 | 1.560 | 0.582 | 0.068 | 2.786E−80 |
| GPC3 | 5.13E−134 | −1.127 | 0.004 | 0.589 | 5.918E−130 | SRP14 | 2.56E−84 | 1.074 | 0.624 | 0.485 | 2.954E−80 |
| EEF1D | 1.73E−133 | −1.405 | 0.274 | 0.91 | 1.994E−129 | MRPL36 | 3.91E−83 | 1.031 | 0.49 | 0.327 | 4.515E−79 |
| HSPA1B | 3.67E−133 | −1.290 | 0.043 | 0.701 | 4.237E−129 | ACTB | 1.60E−82 | 0.974 | 0.686 | 0.608 | 1.845E−78 |
| RPL7A | 7.89E−132 | −1.323 | 0.487 | 0.944 | 9.105E−128 | SSR4 | 2.77E−82 | 1.038 | 0.812 | 0.727 | 3.198E−78 |
| NPDC1 | 1.86E−131 | −0.823 | 0.01 | 0.6 | 2.148E−127 | UGP2 | 2.35E−81 | 0.964 | 0.548 | 0.434 | 2.709E−77 |
| RPS10 | 3.82E−130 | −1.283 | 0.169 | 0.862 | 4.407E−126 | SOD1 | 6.17E−81 | 1.088 | 0.551 | 0.386 | 7.126E−77 |
| PPDPF | 1.26E−129 | −1.330 | 0.148 | 0.845 | 1.451E−125 | AMD1 | 2.36E−79 | 1.186 | 0.51 | 0.242 | 2.729E−75 |
| SPDEF | 4.31E−129 | −0.753 | 0.008 | 0.58 | 4.969E−125 | TXN | 5.66E−79 | 0.938 | 0.721 | 0.676 | 6.528E−75 |
| TMEM256 | 1.24E−128 | −0.811 | 0.012 | 0.586 | 1.435E−124 | TMCO1 | 1.77E−78 | 1.140 | 0.528 | 0.318 | 2.047E−74 |
| ORMDL3 | 1.69E−128 | −0.856 | 0.024 | 0.637 | 1.955E−124 | TAGLN2 | 3.31E−78 | 0.987 | 0.535 | 0.346 | 3.817E−74 |
| RPLP1 | 1.47E−126 | −1.130 | 0.943 | 0.994 | 1.701E−122 | ITGB1 | 4.28E−78 | 1.153 | 0.534 | 0.251 | 4.941E−74 |
| MT-TE | 1.80E−125 | −1.483 | 0.009 | 0.572 | 2.077E−121 | IFT57 | 3.03E−77 | 1.282 | 0.504 | 0.186 | 3.501E−73 |
| GATA3 | 5.88E−124 | −1.219 | 0.041 | 0.656 | 6.786E−120 | SNRPE | 5.27E−77 | 0.921 | 0.484 | 0.397 | 6.088E−73 |
| MT2A | 8.52E−124 | −1.348 | 0.04 | 0.645 | 9.836E−120 | TMEM261 | 1.22E−76 | 1.108 | 0.498 | 0.225 | 1.412E−72 |
| RSF1 | 4.85E−123 | −1.124 | 0.107 | 0.786 | 5.597E−119 | S100A10 | 1.25E−76 | 0.954 | 0.553 | 0.437 | 1.445E−72 |
| C6orf48 | 4.21E−122 | −1.170 | 0.083 | 0.744 | 4.855E−118 | CNIH4 | 1.44E−76 | 0.991 | 0.496 | 0.33 | 1.659E−72 |
| CDK12 | 7.76E−122 | −1.060 | 0.076 | 0.724 | 8.951E−118 | KRT23 | 1.50E−76 | 1.399 | 0.498 | 0.039 | 1.726E−72 |
| MLPH | 3.57E−121 | −0.806 | 0.028 | 0.611 | 4.116E−117 | PDCD6 | 6.82E−76 | 0.827 | 0.447 | 0.344 | 7.868E−72 |
| TMEM254 | 8.51E−121 | −0.735 | 0.022 | 0.597 | 9.824E−117 | MTRNR2L3 | 1.51E−75 | 1.156 | 0.607 | 0.445 | 1.738E−71 |
| CASC3 | 2.89E−120 | −0.848 | 0.029 | 0.628 | 3.337E−116 | CFI | 2.23E−75 | 1.342 | 0.458 | 0 | 2.575E−71 |
| PPFIA1 | 6.71E−120 | −0.922 | 0.06 | 0.693 | 7.746E−116 | C3 | 1.17E−74 | 1.288 | 0.465 | 0.017 | 1.346E−70 |
| KAT6B | 1.16E−118 | −0.942 | 0.038 | 0.645 | 1.343E−114 | SLC38A1 | 4.79E−74 | 1.070 | 0.511 | 0.386 | 5.533E−70 |
| STAC2 | 8.35E−118 | −1.299 | 0.01 | 0.555 | 9.635E−114 | TACSTD2 | 7.24E−74 | 0.909 | 0.593 | 0.479 | 8.358E−70 |
| RPS2 | 1.42E−117 | −1.298 | 0.523 | 0.913 | 1.643E−113 | COX7A2 | 7.70E−74 | 0.880 | 0.644 | 0.611 | 8.888E−70 |
| MT-TL1 | 1.15E−116 | −1.465 | 0.025 | 0.592 | 1.322E−112 | PPP1CB | 1.07E−73 | 0.952 | 0.525 | 0.442 | 1.237E−69 |
| DUSP1 | 2.35E−116 | −1.023 | 0.014 | 0.566 | 2.710E−112 | SSR2 | 2.49E−73 | 0.465 | 0.324 | 0.411 | 2.877E−69 |
| SLC40A1 | 2.04E−115 | −0.819 | 0.001 | 0.507 | 2.353E−111 | CENPW | 3.12E−73 | 1.351 | 0.489 | 0.087 | 3.596E−69 |
| GAPDH | 2.71E−115 | −1.215 | 0.702 | 0.966 | 3.127E−111 | ARL6IP5 | 3.25E−73 | 0.894 | 0.51 | 0.434 | 3.750E−69 |
| WDR83OS | 9.27E−115 | −0.892 | 0.08 | 0.718 | 1.070E−110 | H2AFZ | 2.12E−71 | 0.964 | 0.61 | 0.485 | 2.450E−67 |
| MGST1 | 1.79E−114 | −1.129 | 0.167 | 0.825 | 2.069E−110 | C11orf58 | 6.60E−71 | 0.425 | 0.389 | 0.592 | 7.621E−67 |
| RPL5 | 2.15E−114 | −1.211 | 0.576 | 0.944 | 2.477E−110 | CCT6A | 1.83E−70 | 0.556 | 0.416 | 0.515 | 2.114E−66 |
| RPL37A | 2.06E−113 | −1.136 | 0.834 | 0.986 | 2.383E−109 | GSTP1 | 3.70E−70 | 1.213 | 0.471 | 0.028 | 4.269E−66 |
| CPNE3 | 2.18E−113 | −0.883 | 0.071 | 0.687 | 2.513E−109 | PERP | 3.73E−70 | 0.639 | 0.453 | 0.501 | 4.300E−66 |
| NTPCR | 8.87E−112 | −4.227 | 0.05 | 0.637 | 1.024E−107 | SKP1 | 9.88E−70 | 0.377 | 0.437 | 0.654 | 1.140E−65 |
| CCDC6 | 2.51E−111 | −0.824 | 0.113 | 0.752 | 2.900E−107 | TPM1 | 1.46E−69 | 1.020 | 0.684 | 0.563 | 1.681E−65 |
| SMIM14 | 8.86E−111 | −0.873 | 0.077 | 0.693 | 1.023E−106 | CD63 | 2.22E−69 | 0.710 | 0.478 | 0.479 | 2.565E−65 |
| NEAT1 | 9.90E−111 | −1.191 | 0.102 | 0.71 | 1.143E−106 | IDH2 | 3.81E−69 | 0.830 | 0.443 | 0.352 | 4.397E−65 |
| JMJD1C | 1.36E−109 | −1.115 | 0.16 | 0.792 | 1.569E−105 | NDUFA6 | 5.48E−69 | 0.891 | 0.582 | 0.499 | 6.326E−65 |
| RP11- | 7.43E−109 | −0.846 | 0.068 | 0.665 | 8.578E−105 | SUB1 | 6.56E−69 | 0.699 | 0.501 | 0.524 | 7.572E−65 |
| 304L19.5 | | | | | | | | | | | |
| RPS16 | 1.41E−108 | −1.114 | 0.744 | 0.975 | 1.622E−104 | KRT7 | 8.09E−69 | 0.846 | 0.614 | 0.561 | 9.336E−65 |
| UPF2 | 1.48E−108 | −0.859 | 0.082 | 0.69 | 1.704E−104 | ARPC2 | 1.25E−68 | 0.529 | 0.425 | 0.563 | 1.446E−64 |
| RPS6 | 3.64E−108 | −1.075 | 0.972 | 0.997 | 4.206E−104 | CD46 | 1.30E−68 | 0.879 | 0.44 | 0.33 | 1.501E−64 |
| RPS3A | 2.13E−107 | −0.978 | 0.113 | 0.73 | 2.463E−103 | SUMO2 | 3.98E−68 | 0.498 | 0.382 | 0.476 | 4.592E−64 |
| TFAP2A | 3.22E−107 | −0.832 | 0.05 | 0.628 | 3.716E−103 | ARF1 | 4.69E−68 | 0.806 | 0.605 | 0.561 | 5.408E−64 |
| S100A14 | 1.62E−106 | −1.025 | 0.097 | 0.71 | 1.875E−102 | HSBP1 | 9.02E−68 | 0.751 | 0.547 | 0.555 | 1.041E−63 |
| RPS15A | 7.73E−106 | −1.193 | 0.8 | 0.966 | 8.927E−102 | COX5B | 1.29E−67 | 0.847 | 0.584 | 0.521 | 1.486E−63 |
| TMEM66 | 5.83E−105 | −0.556 | 0.079 | 0.642 | 6.724E−101 | MT-CYB | 2.64E−67 | 1.020 | 0.878 | 0.786 | 3.047E−63 |
| ERGIC1 | 8.23E−105 | −0.761 | 0.058 | 0.637 | 9.504E−101 | MT-ATP6 | 2.88E−67 | 1.001 | 0.857 | 0.738 | 3.323E−63 |
| RNU6-6P | 9.04E−105 | −1.120 | 0.09 | 0.685 | 1.044E−100 | HMGN1 | 3.02E−67 | 0.567 | 0.47 | 0.552 | 3.483E−63 |
| DNAJB1 | 6.24E−104 | −1.096 | 0.142 | 0.758 | 7.205E−100 | HLA-C | 3.43E−67 | 0.870 | 0.569 | 0.434 | 3.954E−63 |
| RPL37 | 8.75E−104 | −1.100 | 0.695 | 0.966 | 1.010E−99 | TOMM20 | 6.11E−67 | 0.693 | 0.592 | 0.614 | 7.055E−63 |
| GRINA | 1.10E−103 | −0.621 | 0.032 | 0.566 | 1.269E−99 | MIF | 6.89E−67 | 0.699 | 0.554 | 0.566 | 7.957E−63 |
| VHL | 5.61E−103 | −0.642 | 0.021 | 0.535 | 6.471E−99 | C11orf31 | 7.17E−67 | 0.425 | 0.298 | 0.392 | 8.280E−63 |
| METTL12 | 7.83E−103 | −0.790 | 0.034 | 0.569 | 9.036E−99 | ANXA1 | 2.80E−66 | 1.358 | 0.516 | 0.101 | 3.228E−62 |
| PRDX2 | 1.19E−102 | −0.996 | 0.109 | 0.718 | 1.377E−98 | DSG2 | 7.01E−66 | 1.002 | 0.405 | 0.144 | 8.088E−62 |
| CLNS1A | 1.50E−102 | −0.716 | 0.093 | 0.679 | 1.734E−98 | MTRNR2L2 | 1.51E−65 | 1.132 | 0.538 | 0.29 | 1.744E−61 |
| EIF4EBP1 | 2.84E−102 | −0.793 | 0.047 | 0.589 | 3.278E−98 | EID1 | 2.25E−65 | 0.340 | 0.28 | 0.485 | 2.598E−61 |
| WWP1 | 3.52E−102 | −0.711 | 0.018 | 0.524 | 4.066E−98 | VIM | 2.34E−65 | 1.202 | 0.508 | 0.051 | 2.704E−61 |
| SERHL2 | 3.24E−101 | −0.655 | 0.003 | 0.468 | 3.735E−97 | TMEM183A | 6.26E−65 | 0.799 | 0.415 | 0.335 | 7.226E−61 |
| ALDH3B2 | 7.76E−101 | −0.614 | 0.006 | 0.473 | 8.959E−97 | PSMA6 | 1.00E−64 | 0.482 | 0.342 | 0.448 | 1.157E−60 |
| FTH1 | 2.05E−100 | −1.277 | 0.569 | 0.935 | 2.371E−96 | CHCHD2 | 2.07E−64 | 0.429 | 0.321 | 0.442 | 2.394E−60 |
| LMO4 | 2.16E−100 | −0.858 | 0.058 | 0.625 | 2.489E−96 | GPNMB | 2.11E−64 | 1.142 | 0.402 | 0.065 | 2.436E−60 |
| CHCHD1 | 7.92E−100 | −0.783 | 0.119 | 0.721 | 9.136E−96 | TSPO | 2.89E−64 | 0.436 | 0.389 | 0.532 | 3.331E−60 |
| JUND | 1.18E−99 | −0.821 | 0.025 | 0.532 | 1.365E−95 | FKBP3 | 4.22E−64 | 0.388 | 0.29 | 0.431 | 4.867E−60 |
| SYNGR2 | 1.48E−98 | −1.194 | 0.264 | 0.851 | 1.714E−94 | UQCRH | 5.46E−64 | 0.515 | 0.54 | 0.656 | 6.297E−60 |
| MYL12B | 2.00E−98 | −1.158 | 0.487 | 0.93 | 2.313E−94 | PPIA | 9.26E−64 | 0.652 | 0.491 | 0.524 | 1.068E−59 |
| RPS7 | 4.61E−98 | −1.192 | 0.226 | 0.823 | 5.325E−94 | UQCR10 | 1.16E−63 | 0.270 | 0.358 | 0.592 | 1.342E−59 |
| NQO1 | 7.00E−98 | −0.650 | 0.052 | 0.586 | 8.074E−94 | PDCD10 | 1.36E−63 | 0.933 | 0.465 | 0.366 | 1.566E−59 |
| VTRNA1-3 | 1.19E−97 | −1.098 | 0.018 | 0.507 | 1.373E−93 | APP | 2.39E−63 | 0.613 | 0.435 | 0.456 | 2.754E−59 |
| RPL18A | 1.16E−96 | −0.906 | 0.134 | 0.738 | 1.344E−92 | ATP5G3 | 2.61E−63 | 0.499 | 0.387 | 0.493 | 3.007E−59 |
| ZMIZ1 | 4.44E−96 | −0.626 | 0.028 | 0.524 | 5.129E−92 | SEC62 | 2.71E−63 | 0.424 | 0.345 | 0.487 | 3.131E−59 |
| CA12 | 5.18E−96 | −0.558 | 0 | 0.425 | 5.982E−92 | TWF1 | 4.73E−63 | 0.818 | 0.355 | 0.22 | 5.457E−59 |
| RHOB | 2.16E−95 | −0.657 | 0.05 | 0.566 | 2.494E−91 | ILF2 | 1.50E−62 | 0.890 | 0.377 | 0.2 | 1.732E−58 |
| RPL28 | 2.15E−94 | −1.080 | 0.46 | 0.907 | 2.481E−90 | KIAA0020 | 1.61E−62 | 1.144 | 0.487 | 0.245 | 1.863E−58 |
| SPINK8 | 3.73E−94 | −0.633 | 0.009 | 0.456 | 4.304E−90 | BCAP31 | 2.03E−62 | 0.273 | 0.313 | 0.507 | 2.345E−58 |
| METRN | 2.32E−93 | −0.620 | 0.035 | 0.524 | 2.681E−89 | CLDN7 | 3.79E−62 | 0.313 | 0.239 | 0.392 | 4.371E−58 |
| PABPC1 | 3.09E−93 | −0.949 | 0.897 | 0.98 | 3.565E−89 | RBP1 | 4.45E−62 | 1.103 | 0.407 | 0.062 | 5.137E−58 |
| ASPH | 1.86E−92 | −0.751 | 0.076 | 0.623 | 2.142E−88 | PAPOLA | 4.76E−62 | 0.487 | 0.355 | 0.459 | 5.495E−58 |
| HNRNPA1 | 8.50E−92 | −0.609 | 0.09 | 0.625 | 9.804E−88 | OST4 | 5.12E−62 | 0.323 | 0.489 | 0.701 | 5.908E−58 |
| ANO1 | 1.11E−91 | −0.544 | 0.008 | 0.445 | 1.286E−87 | TMEM165 | 7.33E−62 | 0.761 | 0.445 | 0.403 | 8.456E−58 |
| IER3 | 2.90E−91 | −0.595 | 0.022 | 0.493 | 3.343E−87 | LMNA | 1.07E−61 | 0.384 | 0.354 | 0.47 | 1.232E−57 |
| TOB1 | 4.64E−91 | −0.734 | 0.103 | 0.648 | 5.361E−87 | HNRNPK | 2.20E−61 | 0.503 | 0.495 | 0.639 | 2.538E−57 |
| RPL15 | 5.59E−91 | −1.038 | 0.682 | 0.938 | 6.455E−87 | ISG15 | 2.33E−61 | 1.077 | 0.481 | 0.096 | 2.686E−57 |
| ZFP36 | 6.39E−91 | −0.648 | 0.03 | 0.515 | 7.376E−87 | CCT3 | 2.79E−61 | 0.716 | 0.404 | 0.386 | 3.218E−57 |
| ENPP1 | 1.27E−90 | −0.599 | 0.005 | 0.439 | 1.469E−86 | ATP5J2 | 3.67E−61 | 0.269 | 0.263 | 0.462 | 4.238E−57 |
| PSMD3 | 2.67E−90 | −0.560 | 0.061 | 0.566 | 3.083E−86 | SERP1 | 6.11E−61 | 0.316 | 0.371 | 0.563 | 7.047E−57 |
| GOLGA2 | 4.54E−90 | −0.635 | 0.073 | 0.597 | 5.245E−86 | CAP1 | 6.29E−61 | 0.573 | 0.403 | 0.487 | 7.256E−57 |
| ESRP1 | 1.38E−89 | −0.541 | 0.08 | 0.592 | 1.595E−85 | HNRNPA2B1 | 8.00E−61 | 0.910 | 0.788 | 0.732 | 9.237E−57 |
| GAS5 | 2.37E−89 | −1.002 | 0.254 | 0.828 | 2.735E−85 | SSR3 | 1.25E−60 | 0.356 | 0.397 | 0.555 | 1.447E−56 |
| RANBP9 | 6.39E−89 | −0.487 | 0.049 | 0.532 | 7.379E−85 | RBX1 | 2.78E−60 | 0.354 | 0.333 | 0.501 | 3.209E−56 |
| RPL27A | 8.19E−89 | −0.980 | 0.727 | 0.963 | 9.453E−85 | PDIA6 | 3.91E−60 | 0.529 | 0.455 | 0.552 | 4.511E−56 |
| CCDC124 | 1.28E−88 | −0.585 | 0.043 | 0.535 | 1.479E−84 | GTF3A | 4.15E−60 | 0.941 | 0.412 | 0.217 | 4.789E−56 |
| HK1 | 1.82E−88 | −0.538 | 0.044 | 0.527 | 2.099E−84 | ATP5H | 4.92E−60 | 0.436 | 0.446 | 0.58 | 5.680E−56 |
| NOL7 | 5.20E−88 | −0.943 | 0.236 | 0.831 | 6.000E−84 | SEC61B | 5.84E−60 | 0.449 | 0.333 | 0.414 | 6.740E−56 |
| GID8 | 1.22E−87 | −0.471 | 0.082 | 0.577 | 1.407E−83 | VIMP | 6.40E−60 | 0.856 | 0.497 | 0.383 | 7.385E−56 |
| VPS26A | 1.55E−87 | −0.736 | 0.127 | 0.682 | 1.784E−83 | NDUFB2 | 7.42E−60 | 0.423 | 0.383 | 0.518 | 8.567E−56 |
| CAST | 2.18E−87 | −0.739 | 0.135 | 0.696 | 2.514E−83 | VAMP8 | 7.67E−60 | 0.524 | 0.418 | 0.51 | 8.851E−56 |
| SCAND1 | 5.35E−87 | −0.452 | 0.074 | 0.549 | 6.179E−83 | 7-Sep | 7.81E−60 | 0.685 | 0.439 | 0.428 | 9.013E−56 |
| H2AFJ | 7.00E−87 | −0.731 | 0.156 | 0.715 | 8.074E−83 | FXR1 | 1.03E−59 | 0.437 | 0.354 | 0.515 | 1.187E−55 |
| RASD1 | 1.60E−86 | −0.616 | 0 | 0.389 | 1.843E−82 | PSMB5 | 1.14E−59 | 0.474 | 0.292 | 0.321 | 1.319E−55 |
| TSG101 | 3.24E−86 | −0.445 | 0.116 | 0.611 | 3.740E−82 | SET | 1.19E−59 | 0.545 | 0.547 | 0.631 | 1.377E−55 |
| C8orf59 | 4.92E−86 | −0.702 | 0.134 | 0.693 | 5.674E−82 | ANXA2 | 1.38E−59 | 0.548 | 0.557 | 0.628 | 1.590E−55 |
| CIRBP | 5.86E−86 | −0.513 | 0.045 | 0.521 | 6.766E−82 | ARGLU1 | 1.60E−59 | 0.843 | 0.395 | 0.313 | 1.849E−55 |
| C19orf43 | 9.05E−86 | −0.622 | 0.119 | 0.662 | 1.044E−81 | SNRPD1 | 3.00E−59 | 0.345 | 0.263 | 0.408 | 3.466E−55 |
| KCNMA1 | 2.06E−85 | −0.677 | 0.009 | 0.434 | 2.374E−81 | CYCS | 3.45E−59 | 0.755 | 0.433 | 0.363 | 3.979E−55 |
| CAPN7 | 3.41E−85 | −0.494 | 0.029 | 0.465 | 3.932E−81 | CCNI | 3.80E−59 | 0.518 | 0.486 | 0.597 | 4.391E−55 |
| PFDN6 | 5.23E−85 | −0.493 | 0.073 | 0.555 | 6.034E−81 | PSME1 | 4.12E−59 | 0.416 | 0.274 | 0.366 | 4.750E−55 |
| FGD5-AS1 | 1.19E−84 | −0.627 | 0.109 | 0.645 | 1.374E−80 | CMPK1 | 4.23E−59 | 0.453 | 0.299 | 0.375 | 4.886E−55 |
| STARD10 | 1.27E−84 | −0.459 | 0.009 | 0.42 | 1.466E−80 | OAZ1 | 4.61E−59 | 0.598 | 0.499 | 0.563 | 5.325E−55 |
| PDXDC1 | 1.47E−84 | −0.535 | 0.089 | 0.594 | 1.693E−80 | PRELID1 | 4.92E−59 | 0.374 | 0.279 | 0.389 | 5.677E−55 |
| SPCS2 | 4.02E−84 | −0.375 | 0.084 | 0.544 | 4.644E−80 | AIMP1 | 5.38E−59 | 0.332 | 0.277 | 0.462 | 6.214E−55 |
| DUSP4 | 4.66E−84 | −0.564 | 0.008 | 0.42 | 5.379E−80 | LGALS3 | 1.23E−58 | 0.558 | 0.458 | 0.544 | 1.419E−54 |
| FRG1 | 3.78E−83 | −0.487 | 0.062 | 0.518 | 4.364E−79 | OAT | 1.43E−58 | 0.360 | 0.302 | 0.442 | 1.645E−54 |
| TMEM141 | 1.16E−82 | −0.489 | 0.123 | 0.62 | 1.343E−78 | CLDN1 | 2.68E−58 | 1.061 | 0.373 | 0 | 3.089E−54 |
| FBXL20 | 6.63E−82 | −0.424 | 0.005 | 0.394 | 7.655E−78 | WDR45B | 2.79E−58 | 0.594 | 0.363 | 0.369 | 3.217E−54 |
| EFHD1 | 7.51E−82 | −0.409 | 0.019 | 0.42 | 8.668E−78 | SMARCA5 | 3.38E−58 | 0.293 | 0.288 | 0.51 | 3.898E−54 |
| PITRM1 | 1.25E−81 | −0.551 | 0.063 | 0.532 | 1.443E−77 | EIF2AK2 | 3.71E−58 | 0.847 | 0.409 | 0.315 | 4.280E−54 |
| RAB6A | 2.25E−81 | −0.517 | 0.067 | 0.544 | 2.600E−77 | RHOA | 4.05E−58 | 0.536 | 0.487 | 0.563 | 4.676E−54 |
| SAMD8 | 2.72E−81 | −0.482 | 0.023 | 0.454 | 3.142E−77 | COX7C | 4.25E−58 | 0.716 | 0.728 | 0.789 | 4.903E−54 |
| PKM | 4.34E−81 | −0.547 | 0.158 | 0.673 | 5.010E−77 | EPRS | 4.84E−58 | 0.404 | 0.346 | 0.51 | 5.589E−54 |
| ECI2 | 4.70E−81 | −0.421 | 0.013 | 0.408 | 5.423E−77 | PRDX4 | 5.39E−58 | 0.617 | 0.361 | 0.346 | 6.222E−54 |
| EIF4A2 | 5.32E−81 | −0.490 | 0.18 | 0.704 | 6.135E−77 | PSMB4 | 5.46E−58 | 0.829 | 0.392 | 0.262 | 6.296E−54 |
| COMTD1 | 7.97E−81 | −0.673 | 0.049 | 0.513 | 9.203E−77 | MRPS21 | 6.57E−58 | 0.517 | 0.259 | 0.287 | 7.579E−54 |
| THRSP | 8.02E−81 | −0.529 | 0 | 0.366 | 9.253E−77 | CRABP2 | 7.03E−58 | 0.708 | 0.436 | 0.425 | 8.114E−54 |
| RPS25 | 8.33E−81 | −1.052 | 0.418 | 0.885 | 9.611E−77 | SAP18 | 1.11E−57 | 0.491 | 0.264 | 0.296 | 1.284E−53 |
| KDM2A | 2.65E−80 | −0.580 | 0.051 | 0.51 | 3.053E−76 | UBE2D3 | 1.31E−57 | 0.354 | 0.392 | 0.538 | 1.515E−53 |
| TTC3 | 6.01E−80 | −0.507 | 0.129 | 0.631 | 6.938E−76 | ENY2 | 1.48E−57 | 0.419 | 0.437 | 0.583 | 1.712E−53 |
| MSL1 | 9.40E−80 | −0.454 | 0.094 | 0.563 | 1.085E−75 | CALML5 | 1.48E−57 | 1.010 | 0.363 | 0.011 | 1.713E−53 |
| PCBD1 | 1.44E−79 | −0.491 | 0.054 | 0.504 | 1.661E−75 | MT-ND3 | 2.00E−57 | 0.779 | 0.837 | 0.73 | 2.312E−53 |
| PLEKHF2 | 1.47E−79 | −0.520 | 0.03 | 0.465 | 1.701E−75 | RBM3 | 2.57E−57 | 0.451 | 0.344 | 0.425 | 2.962E−53 |
| UQCRB | 1.79E−79 | −0.899 | 0.216 | 0.786 | 2.065E−75 | LSM5 | 3.58E−57 | 0.845 | 0.381 | 0.262 | 4.130E−53 |
| EIF4B | 2.62E−79 | −0.581 | 0.1 | 0.597 | 3.028E−75 | HDAC2 | 5.40E−57 | 0.927 | 0.402 | 0.217 | 6.228E−53 |
| FOXA1 | 3.26E−79 | −0.492 | 0.004 | 0.383 | 3.758E−75 | CBX3 | 1.15E−56 | 0.709 | 0.619 | 0.625 | 1.323E−52 |
| ADK | 3.99E−79 | −0.356 | 0.145 | 0.603 | 4.610E−75 | AZGP1 | 1.22E−56 | 0.785 | 0.718 | 0.572 | 1.409E−52 |
| MDH2 | 4.34E−79 | −0.401 | 0.097 | 0.558 | 5.004E−75 | MRPL14 | 1.69E−56 | 0.355 | 0.255 | 0.386 | 1.951E−52 |
| SEC24C | 4.75E−79 | −0.513 | 0.022 | 0.439 | 5.478E−75 | KIF5B | 1.69E−56 | 0.654 | 0.376 | 0.392 | 1.954E−52 |
| TSPAN15 | 9.95E−79 | −0.477 | 0.031 | 0.459 | 1.148E−74 | HADHA | 1.84E−56 | 0.328 | 0.332 | 0.507 | 2.127E−52 |
| C12orf57 | 1.34E−78 | −0.401 | 0.144 | 0.625 | 1.551E−74 | SF3B1 | 2.60E−56 | 0.337 | 0.339 | 0.527 | 2.998E−52 |
| VTRNA1-2 | 1.87E−78 | −0.587 | 0.004 | 0.377 | 2.159E−74 | PLSCR1 | 3.78E−56 | 0.999 | 0.396 | 0.07 | 4.362E−52 |
| PLA2G16 | 2.76E−78 | −0.468 | 0.045 | 0.468 | 3.184E−74 | ATP5J | 6.02E−56 | 0.587 | 0.325 | 0.355 | 6.944E−52 |
| FASN | 1.00E−77 | −0.750 | 0.068 | 0.555 | 1.160E−73 | PRPF40A | 1.35E−55 | 0.329 | 0.293 | 0.468 | 1.554E−51 |
| ZC2HC1A | 1.57E−77 | −0.452 | 0.013 | 0.4 | 1.806E−73 | TCEB2 | 1.58E−55 | 0.293 | 0.465 | 0.656 | 1.824E−51 |
| SLC39A7 | 1.86E−77 | −0.505 | 0.083 | 0.552 | 2.142E−73 | USP1 | 2.12E−55 | 0.592 | 0.387 | 0.42 | 2.442E−51 |
| EIF3L | 2.84E−77 | −0.599 | 0.147 | 0.662 | 3.280E−73 | EIF4G2 | 2.69E−55 | 0.519 | 0.465 | 0.558 | 3.108E−51 |
| AP3M1 | 3.26E−77 | −0.423 | 0.027 | 0.434 | 3.766E−73 | COX8A | 4.22E−55 | 0.366 | 0.358 | 0.501 | 4.870E−51 |
| CSNK2B | 3.40E−77 | −0.502 | 0.031 | 0.465 | 3.925E−73 | SMS | 7.86E−55 | 0.347 | 0.239 | 0.349 | 9.071E−51 |
| MRPS28 | 1.03E−76 | −0.391 | 0.031 | 0.425 | 1.191E−72 | IFITM3 | 9.59E−55 | 0.897 | 0.449 | 0.189 | 1.107E−50 |
| DHRS2 | 1.30E−76 | −0.401 | 0 | 0.349 | 1.499E−72 | RNF181 | 1.00E−54 | 0.287 | 0.216 | 0.358 | 1.156E−50 |
| AFG3L2 | 2.09E−76 | −0.376 | 0.105 | 0.561 | 2.418E−72 | VBP1 | 1.15E−54 | 0.517 | 0.357 | 0.431 | 1.330E−50 |
| AHNAK | 4.89E−76 | −0.412 | 0.103 | 0.552 | 5.638E−72 | NENF | 2.47E−54 | 0.526 | 0.298 | 0.304 | 2.845E−50 |
| TPD52 | 6.44E−76 | −0.386 | 0.092 | 0.538 | 7.429E−72 | USMG5 | 2.82E−54 | 0.429 | 0.521 | 0.676 | 3.257E−50 |
| SPTSSA | 6.62E−76 | −0.375 | 0.111 | 0.555 | 7.635E−72 | C1orf21 | 2.89E−54 | 0.732 | 0.426 | 0.377 | 3.334E−50 |
| RHOBTB1 | 7.01E−76 | −0.601 | 0.015 | 0.414 | 8.086E−72 | UBE2K | 3.79E−54 | 0.376 | 0.209 | 0.301 | 4.378E−50 |
| CLCA2 | 3.20E−75 | −0.538 | 0 | 0.344 | 3.689E−71 | LGALS1 | 5.39E−54 | 0.995 | 0.649 | 0.434 | 6.217E−50 |
| ZFP36L2 | 3.74E−75 | −0.690 | 0.115 | 0.6 | 4.321E−71 | PSMB7 | 6.67E−54 | 0.319 | 0.318 | 0.459 | 7.695E−50 |
| MT-TS2 | 4.41E−75 | −1.211 | 0.021 | 0.411 | 5.087E−71 | LDHB | 7.35E−54 | 1.028 | 0.361 | 0.008 | 8.484E−50 |
| RPS9 | 4.70E−75 | −0.955 | 0.449 | 0.904 | 5.427E−71 | ARHGDIB | 8.93E−54 | 0.686 | 0.304 | 0.254 | 1.030E−49 |
| NBN | 8.40E−75 | −0.366 | 0.045 | 0.448 | 9.698E−71 | NDUFB4 | 9.63E−54 | 0.292 | 0.261 | 0.403 | 1.112E−49 |
| RMDN1 | 1.28E−74 | −0.338 | 0.025 | 0.386 | 1.481E−70 | SRSF3 | 1.16E−53 | 0.597 | 0.499 | 0.58 | 1.342E−49 |
| TSPAN1 | 1.38E−74 | −0.496 | 0.005 | 0.369 | 1.592E−70 | UBL5 | 1.24E−53 | 0.473 | 0.512 | 0.659 | 1.433E−49 |
| CLDN4 | 3.10E−74 | −0.784 | 0.229 | 0.732 | 3.583E−70 | TMED2 | 1.39E−53 | 0.382 | 0.345 | 0.479 | 1.606E−49 |
| GALNT7 | 4.55E−74 | −0.441 | 0.055 | 0.476 | 5.248E−70 | APEX1 | 1.43E−53 | 0.329 | 0.248 | 0.403 | 1.655E−49 |
| RPS27L | 4.98E−74 | −0.421 | 0.156 | 0.611 | 5.745E−70 | PAICS | 2.66E−53 | 0.370 | 0.272 | 0.369 | 3.066E−49 |
| CCDC57 | 6.37E−74 | −0.322 | 0.027 | 0.4 | 7.357E−70 | HDGF | 3.81E−53 | 0.611 | 0.42 | 0.403 | 4.398E−49 |
| WBSCR22 | 8.79E−74 | −0.258 | 0.082 | 0.47 | 1.014E−69 | ANXA5 | 5.25E−53 | 0.293 | 0.313 | 0.501 | 6.063E−49 |
| CTSD | 1.37E−73 | −0.509 | 0.054 | 0.479 | 1.579E−69 | HNRNPF | 7.65E−53 | 0.252 | 0.276 | 0.462 | 8.831E−49 |
| FOSB | 2.36E−73 | −0.513 | 0.002 | 0.341 | 2.719E−69 | MT-ND4 | 7.88E−53 | 0.850 | 0.956 | 0.935 | 9.097E−49 |
| TSPAN14 | 2.67E−73 | −0.305 | 0.035 | 0.408 | 3.079E−69 | ACTR2 | 8.35E−53 | 0.285 | 0.369 | 0.572 | 9.635E−49 |
| SEPHS2 | 3.16E−73 | −0.328 | 0.075 | 0.487 | 3.644E−69 | CLTA | 9.99E−53 | 0.276 | 0.318 | 0.499 | 1.153E−48 |
| TRMT112 | 3.76E−73 | −0.278 | 0.124 | 0.532 | 4.334E−69 | ITM2B | 1.36E−52 | 0.588 | 0.384 | 0.358 | 1.572E−48 |
| SERTAD1 | 3.80E−73 | −0.390 | 0.006 | 0.369 | 4.380E−69 | MTRNR2L12 | 1.61E−52 | 0.818 | 0.355 | 0.197 | 1.862E−48 |
| MMP24-AS1 | 4.00E−73 | −0.370 | 0.013 | 0.377 | 4.615E−69 | SRP9 | 1.77E−52 | 0.616 | 0.395 | 0.377 | 2.044E−48 |
| EIF1 | 4.45E−73 | −0.862 | 0.395 | 0.901 | 5.135E−69 | TROVE2 | 1.87E−52 | 0.696 | 0.305 | 0.259 | 2.161E−48 |
| MTDH | 6.09E−73 | −0.512 | 0.152 | 0.648 | 7.025E−69 | PHB | 1.99E−52 | 0.433 | 0.25 | 0.265 | 2.297E−48 |
| GADD45GIP1 | 7.10E−73 | −0.750 | 0.235 | 0.786 | 8.191E−69 | MPZL1 | 2.22E−52 | 0.570 | 0.29 | 0.273 | 2.562E−48 |
| PPAPDC1B | 9.80E−73 | −0.416 | 0.01 | 0.375 | 1.131E−68 | TMEM59 | 2.34E−52 | 0.581 | 0.325 | 0.346 | 2.701E−48 |
| GTF2H1 | 1.62E−72 | −0.268 | 0.119 | 0.538 | 1.866E−68 | TRAM1 | 2.58E−52 | 0.709 | 0.347 | 0.304 | 2.972E−48 |
| SNHG3 | 3.49E−72 | −0.504 | 0.032 | 0.437 | 4.028E−68 | LAMP2 | 2.74E−52 | 0.749 | 0.389 | 0.31 | 3.161E−48 |
| PPP1R15A | 6.16E−72 | −0.576 | 0.061 | 0.501 | 7.105E−68 | JTB | 2.85E−52 | 0.628 | 0.463 | 0.425 | 3.284E−48 |
| NDUFB1 | 6.18E−72 | −0.542 | 0.18 | 0.662 | 7.131E−68 | DSC2 | 3.67E−52 | 1.014 | 0.376 | 0.11 | 4.240E−48 |
| DCXR | 6.23E−72 | −0.524 | 0.084 | 0.541 | 7.190E−68 | STMN1 | 4.17E−52 | 0.840 | 0.474 | 0.383 | 4.814E−48 |
| C3orf14 | 6.93E−72 | −0.346 | 0.042 | 0.423 | 7.995E−68 | ANP32B | 4.38E−52 | 0.288 | 0.33 | 0.521 | 5.058E−48 |
| SMIM7 | 2.17E−71 | −0.278 | 0.049 | 0.411 | 2.500E−67 | COX7B | 4.74E−52 | 0.429 | 0.385 | 0.482 | 5.475E−48 |
| HILPDA | 2.87E−71 | −0.557 | 0.032 | 0.437 | 3.310E−67 | FDPS | 5.50E−52 | 0.691 | 0.346 | 0.254 | 6.350E−48 |
| RAD23A | 3.04E−71 | −0.529 | 0.155 | 0.648 | 3.505E−67 | ZCRB1 | 5.81E−52 | 0.623 | 0.327 | 0.346 | 6.703E−48 |
| TMED3 | 3.97E−71 | −0.381 | 0.065 | 0.462 | 4.579E−67 | KHDRBS1 | 6.08E−52 | 0.290 | 0.255 | 0.411 | 7.022E−48 |
| CNTNAP2 | 4.88E−71 | −0.418 | 0.003 | 0.346 | 5.631E−67 | SRP72 | 6.29E−52 | 0.507 | 0.321 | 0.389 | 7.256E−48 |
| AAMDC | 5.28E−71 | −0.375 | 0.019 | 0.392 | 6.092E−67 | HSPA8 | 6.53E−52 | 0.499 | 0.524 | 0.623 | 7.533E−48 |
| TFAM | 7.65E−71 | −0.418 | 0.102 | 0.544 | 8.826E−67 | CSTB | 6.76E−52 | 0.524 | 0.418 | 0.468 | 7.801E−48 |
| FAM210B | 1.01E−70 | −0.558 | 0.104 | 0.572 | 1.167E−66 | SVIP | 9.18E−52 | 0.767 | 0.416 | 0.307 | 1.059E−47 |
| S100A9 | 1.27E−70 | −1.229 | 0.166 | 0.594 | 1.461E−66 | CTSS | 9.55E−52 | 1.097 | 0.407 | 0.037 | 1.102E−47 |
| VAV3 | 2.28E−70 | −0.377 | 0.002 | 0.335 | 2.627E−66 | ACP1 | 9.94E−52 | 0.293 | 0.299 | 0.473 | 1.147E−47 |
| ALDOA | 3.10E−70 | −0.971 | 0.449 | 0.887 | 3.574E−66 | MRPL13 | 1.10E−51 | 0.497 | 0.35 | 0.411 | 1.265E−47 |
| NGLY1 | 3.47E−70 | −0.311 | 0.016 | 0.363 | 4.001E−66 | CHMP2B | 1.17E−51 | 0.300 | 0.266 | 0.408 | 1.350E−47 |
| RPL18 | 5.80E−70 | −0.927 | 0.417 | 0.879 | 6.695E−66 | HMGB1 | 1.22E−51 | 0.679 | 0.505 | 0.49 | 1.404E−47 |
| XPC | 7.33E−70 | −0.425 | 0.029 | 0.411 | 8.464E−66 | UBE2I | 1.54E−51 | 0.466 | 0.276 | 0.31 | 1.781E−47 |
| NUDT8 | 7.92E−70 | −0.526 | 0.024 | 0.406 | 9.140E−66 | GHITM | 2.10E−51 | 0.396 | 0.281 | 0.38 | 2.427E−47 |
| VPS4B | 7.99E−70 | −0.285 | 0.124 | 0.51 | 9.226E−66 | CAPRIN1 | 2.66E−51 | 0.526 | 0.287 | 0.307 | 3.075E−47 |
| GLTSCR2 | 8.01E−70 | −0.518 | 0.15 | 0.625 | 9.247E−66 | LMAN1 | 2.89E−51 | 0.271 | 0.371 | 0.532 | 3.334E−47 |
| RN7SK | 8.61E−70 | −0.689 | 0.015 | 0.38 | 9.938E−66 | GNB1 | 3.69E−51 | 0.293 | 0.25 | 0.389 | 4.260E−47 |
| RNA28S5 | 1.31E−69 | −1.049 | 0.528 | 0.927 | 1.508E−65 | MORF4L2 | 3.95E−51 | 0.366 | 0.344 | 0.515 | 4.557E−47 |
| PPP1CA | 1.33E−69 | −0.277 | 0.116 | 0.513 | 1.539E−65 | PTGES3 | 4.38E−51 | 0.341 | 0.383 | 0.504 | 5.055E−47 |
| RPL23A | 1.70E−69 | −0.888 | 0.319 | 0.834 | 1.967E−65 | SUCLG1 | 4.81E−51 | 0.354 | 0.329 | 0.425 | 5.549E−47 |
| COX5A | 1.93E−69 | −0.338 | 0.144 | 0.572 | 2.225E−65 | ACTR3 | 5.19E−51 | 0.683 | 0.355 | 0.307 | 5.991E−47 |
| COA4 | 1.99E−69 | −0.342 | 0.082 | 0.485 | 2.291E−65 | TPR | 5.90E−51 | 0.574 | 0.306 | 0.324 | 6.810E−47 |
| SEC13 | 4.03E−69 | −0.389 | 0.105 | 0.535 | 4.651E−65 | NDUFA1 | 5.93E−51 | 0.259 | 0.417 | 0.659 | 6.846E−47 |
| SPTY2D1 | 4.65E−69 | −0.471 | 0.175 | 0.645 | 5.362E−65 | MAP3K13 | 6.03E−51 | 0.296 | 0.232 | 0.372 | 6.957E−47 |
| SLK | 4.91E−69 | −0.445 | 0.103 | 0.535 | 5.661E−65 | LAMP1 | 6.49E−51 | 0.366 | 0.278 | 0.38 | 7.488E−47 |
| ODC1 | 5.31E−69 | −0.721 | 0.027 | 0.425 | 6.125E−65 | DDX17 | 7.20E−51 | 0.279 | 0.445 | 0.648 | 8.304E−47 |
| DANCR | 8.62E−69 | −0.435 | 0.121 | 0.569 | 9.945E−65 | RGS10 | 7.91E−51 | 0.764 | 0.381 | 0.256 | 9.123E−47 |
| BZW1 | 1.11E−68 | −0.392 | 0.144 | 0.594 | 1.285E−64 | RPN1 | 9.28E−51 | 0.396 | 0.227 | 0.285 | 1.071E−46 |
| TRIB3 | 1.40E−68 | −0.483 | 0.011 | 0.369 | 1.613E−64 | HAP1 | 1.13E−50 | 0.953 | 0.374 | 0.025 | 1.304E−46 |
| SNHG6 | 1.52E−68 | −0.370 | 0.205 | 0.656 | 1.751E−64 | IDI1 | 1.46E−50 | 0.812 | 0.384 | 0.231 | 1.686E−46 |
| RPL27 | 3.20E−68 | −0.869 | 0.719 | 0.958 | 3.695E−64 | SQLE | 1.55E−50 | 0.588 | 0.387 | 0.397 | 1.794E−46 |
| KRT18 | 3.23E−68 | −0.655 | 0.265 | 0.775 | 3.723E−64 | SF3B14 | 2.09E−50 | 0.456 | 0.301 | 0.377 | 2.414E−46 |
| NDUFAF6 | 3.60E−68 | −0.284 | 0.019 | 0.358 | 4.157E−64 | PRRC2C | 2.11E−50 | 0.322 | 0.389 | 0.555 | 2.434E−46 |
| ANAPC16 | 4.89E−68 | −0.290 | 0.116 | 0.51 | 5.645E−64 | C1orf43 | 2.13E−50 | 0.536 | 0.305 | 0.338 | 2.455E−46 |
| AFMID | 5.43E−68 | −0.378 | 0.004 | 0.33 | 6.266E−64 | SYNCRIP | 2.14E−50 | 0.742 | 0.35 | 0.231 | 2.472E−46 |
| EIF2S2 | 7.85E−68 | −0.569 | 0.211 | 0.715 | 9.064E−64 | H2AFV | 2.24E−50 | 0.416 | 0.274 | 0.349 | 2.588E−46 |
| LSM3 | 1.10E−67 | −0.696 | 0.238 | 0.763 | 1.275E−63 | CALM2 | 2.25E−50 | 0.772 | 0.776 | 0.761 | 2.592E−46 |
| FGD5 | 1.12E−67 | −0.387 | 0 | 0.313 | 1.293E−63 | ENO1 | 2.39E−50 | 0.582 | 0.718 | 0.755 | 2.758E−46 |
| SUSD2 | 5.38E−67 | −0.361 | 0.001 | 0.321 | 6.214E−63 | TBL1XR1 | 2.43E−50 | 0.457 | 0.32 | 0.383 | 2.800E−46 |
| ZMYND8 | 6.17E−67 | −0.448 | 0.051 | 0.442 | 7.117E−63 | RCN1 | 2.61E−50 | 0.719 | 0.318 | 0.208 | 3.012E−46 |
| SERPINB6 | 6.55E−67 | −0.307 | 0.032 | 0.377 | 7.557E−63 | DPM3 | 2.75E−50 | 0.338 | 0.232 | 0.346 | 3.171E−46 |
| NDRG1 | 6.93E−67 | −0.901 | 0.041 | 0.442 | 7.996E−63 | MX1 | 2.78E−50 | 1.055 | 0.426 | 0.079 | 3.210E−46 |
| KRT10 | 7.22E−67 | −0.277 | 0.024 | 0.358 | 8.338E−63 | GGCT | 2.80E−50 | 0.275 | 0.448 | 0.673 | 3.231E−46 |
| TRIQK | 1.84E−66 | −0.361 | 0.011 | 0.341 | 2.118E−62 | DYNLL1 | 3.14E−50 | 0.402 | 0.374 | 0.448 | 3.619E−46 |
| CPT1A | 2.21E−66 | −0.395 | 0.01 | 0.346 | 2.556E−62 | DDX21 | 4.72E−50 | 0.277 | 0.264 | 0.425 | 5.446E−46 |
| RALBP1 | 2.36E−66 | −0.358 | 0.141 | 0.558 | 2.725E−62 | SLIRP | 5.01E−50 | 0.332 | 0.32 | 0.459 | 5.781E−46 |
| CSNK1A1 | 3.66E−66 | −0.440 | 0.227 | 0.701 | 4.229E−62 | AURKAIP1 | 7.95E−50 | 0.383 | 0.22 | 0.265 | 9.175E−46 |
| FBL | 4.20E−66 | −0.321 | 0.097 | 0.485 | 4.852E−62 | IARS2 | 8.55E−50 | 0.703 | 0.346 | 0.313 | 9.865E−46 |
| RNF152 | 8.33E−66 | −0.307 | 0.008 | 0.327 | 9.610E−62 | CD99 | 1.04E−49 | 0.550 | 0.256 | 0.217 | 1.199E−45 |
| LINC00657 | 1.34E−65 | −0.467 | 0.093 | 0.515 | 1.542E−61 | SCAF11 | 1.13E−49 | 0.520 | 0.345 | 0.437 | 1.308E−45 |
| CTSF | 1.85E−65 | −0.343 | 0.009 | 0.335 | 2.135E−61 | SBDS | 1.68E−49 | 0.291 | 0.2 | 0.307 | 1.943E−45 |
| FAM32A | 2.04E−65 | −0.373 | 0.096 | 0.501 | 2.359E−61 | TPT1 | 2.37E−49 | 0.777 | 0.964 | 0.938 | 2.732E−45 |
| PPIF | 2.91E−65 | −0.429 | 0.05 | 0.431 | 3.360E−61 | DYNLT1 | 2.98E−49 | 0.447 | 0.317 | 0.361 | 3.434E−45 |
| EIF3M | 4.24E−65 | −0.341 | 0.232 | 0.682 | 4.890E−61 | DHX9 | 3.61E−49 | 0.583 | 0.308 | 0.279 | 4.164E−45 |
| DDIT4 | 4.68E−65 | −0.522 | 0.108 | 0.521 | 5.404E−61 | U2SURP | 4.91E−49 | 0.312 | 0.315 | 0.501 | 5.667E−45 |
| CDK2AP2 | 5.72E−65 | −0.282 | 0.015 | 0.335 | 6.600E−61 | PTTG1IP | 5.11E−49 | 0.486 | 0.499 | 0.558 | 5.896E−45 |
| RABAC1 | 6.09E−65 | −0.325 | 0.061 | 0.431 | 7.028E−61 | AKAP9 | 6.12E−49 | 0.482 | 0.365 | 0.51 | 7.063E−45 |
| MRPL41 | 6.22E−65 | −0.327 | 0.116 | 0.504 | 7.181E−61 | CALM1 | 7.20E−49 | 0.587 | 0.555 | 0.592 | 8.309E−45 |
| VTRNA1-1 | 7.06E−65 | −0.494 | 0.002 | 0.313 | 8.148E−61 | NDUFB5 | 7.63E−49 | 0.330 | 0.196 | 0.276 | 8.807E−45 |
| SERGEF | 8.37E−65 | −0.398 | 0.006 | 0.335 | 9.665E−61 | VDAC1 | 9.07E−49 | 0.394 | 0.341 | 0.437 | 1.047E−44 |
| KDELR1 | 8.91E−65 | −0.308 | 0.11 | 0.49 | 1.028E−60 | TOMM7 | 9.34E−49 | 0.494 | 0.551 | 0.659 | 1.078E−44 |
| C17orf89 | 1.10E−64 | −0.272 | 0.179 | 0.586 | 1.271E−60 | MDH1 | 1.11E−48 | 0.457 | 0.258 | 0.296 | 1.275E−44 |
| PRSS23 | 1.50E−64 | −0.528 | 0.017 | 0.377 | 1.727E−60 | NDUFB11 | 1.72E−48 | 0.456 | 0.303 | 0.358 | 1.989E−44 |
| NELFE | 1.65E−64 | −0.319 | 0.089 | 0.468 | 1.908E−60 | RAB10 | 1.88E−48 | 0.326 | 0.302 | 0.403 | 2.167E−44 |
| COMT | 2.33E−64 | −0.277 | 0.092 | 0.468 | 2.687E−60 | EIF3E | 2.37E−48 | 0.316 | 0.444 | 0.617 | 2.736E−44 |
| POR | 2.39E−64 | −0.259 | 0.029 | 0.358 | 2.758E−60 | SNRPG | 3.04E−48 | 0.293 | 0.294 | 0.434 | 3.505E−44 |
| SERINC2 | 3.12E−64 | −0.326 | 0.061 | 0.431 | 3.599E−60 | MRPS18C | 3.35E−48 | 0.758 | 0.287 | 0.183 | 3.864E−44 |
| RPN2 | 3.19E−64 | −0.410 | 0.108 | 0.527 | 3.685E−60 | PGRMC1 | 3.57E−48 | 0.645 | 0.324 | 0.293 | 4.122E−44 |
| FADD | 5.28E−64 | −0.251 | 0.04 | 0.366 | 6.089E−60 | TOR1AIP2 | 4.13E−48 | 0.607 | 0.279 | 0.211 | 4.762E−44 |
| C8orf4 | 7.80E−64 | −0.690 | 0.001 | 0.307 | 9.000E−60 | DBI | 4.26E−48 | 0.683 | 0.667 | 0.651 | 4.913E−44 |
| C6orf62 | 1.04E−63 | −0.345 | 0.106 | 0.487 | 1.199E−59 | ZFP36L1 | 4.85E−48 | 0.263 | 0.242 | 0.372 | 5.597E−44 |
| ANKRD30B | 1.24E−63 | −0.452 | 0.001 | 0.304 | 1.429E−59 | MUC15 | 4.86E−48 | 0.927 | 0.325 | 0.003 | 5.610E−44 |
| DYNLRB1 | 1.41E−63 | −0.312 | 0.118 | 0.515 | 1.631E−59 | HMGA1 | 8.05E−48 | 0.455 | 0.284 | 0.335 | 9.295E−44 |
| CHMP1B | 1.47E−63 | −0.440 | 0.048 | 0.417 | 1.691E−59 | MAPKAPK2 | 9.44E−48 | 0.738 | 0.333 | 0.177 | 1.089E−43 |
| IMPDH2 | 1.56E−63 | −0.318 | 0.054 | 0.408 | 1.795E−59 | AIF1L | 9.65E−48 | 0.509 | 0.264 | 0.242 | 1.114E−43 |
| ANKRD12 | 1.70E−63 | −0.582 | 0.133 | 0.555 | 1.961E−59 | SRPK1 | 1.27E−47 | 0.416 | 0.227 | 0.279 | 1.468E−43 |
| NOP56 | 3.51E−63 | −0.363 | 0.067 | 0.442 | 4.049E−59 | VMP1 | 1.32E−47 | 0.495 | 0.328 | 0.349 | 1.523E−43 |
| ASNA1 | 3.73E−63 | −0.304 | 0.027 | 0.358 | 4.302E−59 | S100A1 | 1.51E−47 | 0.872 | 0.318 | 0.008 | 1.745E−43 |
| TXNDC17 | 3.90E−63 | −0.332 | 0.157 | 0.572 | 4.500E−59 | CKS2 | 1.84E−47 | 0.493 | 0.274 | 0.296 | 2.127E−43 |
| REEP5 | 3.97E−63 | −0.492 | 0.127 | 0.546 | 4.585E−59 | CD164 | 2.45E−47 | 0.586 | 0.317 | 0.276 | 2.827E−43 |
| MAGEA3 | 5.76E−63 | −0.291 | 0 | 0.293 | 6.642E−59 | NFE2L1 | 2.53E−47 | 0.277 | 0.171 | 0.256 | 2.925E−43 |
| EMP2 | 8.75E−63 | −0.308 | 0.1 | 0.485 | 1.010E−58 | ART3 | 2.68E−47 | 0.968 | 0.329 | 0.003 | 3.092E−43 |
| SRRM2 | 8.92E−63 | −0.355 | 0.137 | 0.541 | 1.030E−58 | STIP1 | 3.09E−47 | 0.297 | 0.208 | 0.307 | 3.564E−43 |
| NDUFV2 | 9.89E−63 | −0.342 | 0.013 | 0.33 | 1.141E−58 | NDUFA5 | 3.19E−47 | 0.381 | 0.19 | 0.268 | 3.683E−43 |
| SARS | 1.19E−62 | −0.365 | 0.08 | 0.465 | 1.371E−58 | ATG3 | 3.94E−47 | 0.688 | 0.279 | 0.197 | 4.546E−43 |
| TRIBI | 1.33E−62 | −0.423 | 0.042 | 0.397 | 1.539E−58 | POMP | 4.13E−47 | 0.650 | 0.361 | 0.31 | 4.772E−43 |
| EIF3H | 1.43E−62 | −0.775 | 0.418 | 0.899 | 1.648E−58 | HSPE1 | 4.22E−47 | 0.544 | 0.497 | 0.499 | 4.866E−43 |
| MORF4L1 | 1.91E−62 | −0.300 | 0.15 | 0.555 | 2.202E−58 | TCP1 | 4.64E−47 | 0.292 | 0.256 | 0.403 | 5.358E−43 |
| DYNC1I2 | 2.10E−62 | −0.326 | 0.104 | 0.482 | 2.425E−58 | CAPG | 5.09E−47 | 0.407 | 0.216 | 0.214 | 5.869E−43 |
| PYCR1 | 2.44E−62 | −0.362 | 0.076 | 0.454 | 2.818E−58 | ATP5C1 | 5.82E−47 | 0.256 | 0.488 | 0.69 | 6.718E−43 |
| HNRNPA0 | 2.49E−62 | −0.337 | 0.082 | 0.462 | 2.875E−58 | HMGB2 | 5.98E−47 | 0.965 | 0.537 | 0.301 | 6.906E−43 |
| LARS | 2.66E−62 | −0.273 | 0.174 | 0.575 | 3.073E−58 | HNRNPC | 6.57E−47 | 0.286 | 0.437 | 0.62 | 7.581E−43 |
| RPS11 | 3.30E−62 | −0.772 | 0.811 | 0.975 | 3.809E−58 | UBE2T | 9.63E−47 | 0.693 | 0.255 | 0.121 | 1.112E−42 |
| CSDE1 | 3.61E−62 | −0.392 | 0.227 | 0.687 | 4.165E−58 | YWHAQ | 1.64E−46 | 0.403 | 0.298 | 0.338 | 1.888E−42 |
| DPP7 | 3.81E−62 | −0.303 | 0.056 | 0.408 | 4.395E−58 | CLTC | 2.22E−46 | 0.325 | 0.286 | 0.408 | 2.565E−42 |
| CHMP4C | 6.23E−62 | −0.342 | 0.037 | 0.366 | 7.195E−58 | AP2M1 | 3.41E−46 | 0.323 | 0.221 | 0.31 | 3.940E−42 |
| CRIP2 | 8.11E−62 | −0.351 | 0.093 | 0.473 | 9.356E−58 | ACTR6 | 3.56E−46 | 0.690 | 0.291 | 0.239 | 4.110E−42 |
| VAPA | 1.20E−61 | −0.433 | 0.244 | 0.701 | 1.379E−57 | PTBP3 | 4.59E−46 | 0.323 | 0.289 | 0.406 | 5.293E−42 |
| RP11- | 1.24E−61 | −0.313 | 0 | 0.287 | 1.431E−57 | PTP4A1 | 4.78E−46 | 0.302 | 0.205 | 0.299 | 5.514E−42 |
| 369C8.1 | | | | | | | | | | | |
| RBM17 | 1.55E−61 | −0.425 | 0.209 | 0.656 | 1.785E−57 | MT-ATP8 | 6.77E−46 | 0.670 | 0.584 | 0.58 | 7.815E−42 |
| NFIA | 5.87E−61 | −0.287 | 0.1 | 0.47 | 6.770E−57 | PARP1 | 7.68E−46 | 0.351 | 0.218 | 0.321 | 8.862E−42 |
| OXSM | 7.22E−61 | −0.256 | 0.018 | 0.321 | 8.337E−57 | LAGE3 | 8.34E−46 | 0.256 | 0.17 | 0.282 | 9.620E−42 |
| FKBP1A | 7.55E−61 | −0.367 | 0.031 | 0.38 | 8.714E−57 | SNX6 | 9.29E−46 | 0.369 | 0.229 | 0.315 | 1.072E−41 |
| UBC | 8.00E−61 | −0.668 | 0.34 | 0.845 | 9.233E−57 | RAN | 9.48E−46 | 0.360 | 0.4 | 0.515 | 1.094E−41 |
| SNHG9 | 1.00E−60 | −0.291 | 0.057 | 0.403 | 1.160E−56 | PPM1G | 1.63E−45 | 0.492 | 0.38 | 0.411 | 1.883E−41 |
| AQP5 | 1.91E−60 | −0.595 | 0.04 | 0.406 | 2.205E−56 | NOP10 | 2.67E−45 | 0.347 | 0.229 | 0.31 | 3.087E−41 |
| RNA5SP149 | 2.37E−60 | −0.485 | 0.017 | 0.349 | 2.739E−56 | NPC2 | 2.80E−45 | 0.260 | 0.207 | 0.321 | 3.235E−41 |
| RPL31 | 2.95E−60 | −0.753 | 0.79 | 0.963 | 3.400E−56 | MYEOV2 | 4.57E−45 | 0.306 | 0.419 | 0.583 | 5.269E−41 |
| ATF4 | 2.98E−60 | −0.407 | 0.269 | 0.73 | 3.440E−56 | GLRX3 | 6.10E−45 | 0.303 | 0.181 | 0.273 | 7.040E−41 |
| GPX4 | 4.27E−60 | −0.478 | 0.258 | 0.724 | 4.931E−56 | RAD23B | 6.12E−45 | 0.304 | 0.238 | 0.344 | 7.067E−41 |
| SIRT5 | 4.99E−60 | −0.403 | 0.021 | 0.361 | 5.759E−56 | SNRPF | 6.79E−45 | 0.274 | 0.157 | 0.273 | 7.838E−41 |
| PCBP2 | 6.23E−60 | −0.292 | 0.124 | 0.504 | 7.196E−56 | SIVA1 | 7.28E−45 | 0.296 | 0.258 | 0.383 | 8.406E−41 |
| UCP2 | 8.18E−60 | −0.349 | 0.014 | 0.33 | 9.441E−56 | DAP | 9.23E−45 | 0.539 | 0.286 | 0.268 | 1.065E−40 |
| SLC31A1 | 9.36E−60 | −0.293 | 0.06 | 0.408 | 1.081E−55 | SFRP1 | 1.12E−44 | 0.840 | 0.297 | 0.006 | 1.297E−40 |
| NACC1 | 1.26E−59 | −0.290 | 0.017 | 0.341 | 1.451E−55 | GGPS1 | 1.17E−44 | 0.255 | 0.153 | 0.239 | 1.349E−40 |
| GSTM3 | 1.80E−59 | −0.291 | 0.009 | 0.307 | 2.076E−55 | AZINI | 1.18E−44 | 0.261 | 0.174 | 0.268 | 1.367E−40 |
| ETFA | 1.93E−59 | −0.267 | 0.053 | 0.392 | 2.227E−55 | YME1L1 | 1.38E−44 | 0.434 | 0.234 | 0.285 | 1.598E−40 |
| DNASE2 | 1.98E−59 | −0.330 | 0.048 | 0.389 | 2.283E−55 | MEF2A | 1.55E−44 | 0.633 | 0.301 | 0.217 | 1.784E−40 |
| RB1CC1 | 2.01E−59 | −0.432 | 0.083 | 0.459 | 2.320E−55 | DAD1 | 2.06E−44 | 0.367 | 0.253 | 0.341 | 2.376E−40 |
| RPS3 | 2.59E−59 | −0.796 | 0.588 | 0.924 | 2.993E−55 | SPIN1 | 2.35E−44 | 0.432 | 0.187 | 0.186 | 2.709E−40 |
| GOLGB1 | 3.64E−59 | −0.375 | 0.16 | 0.561 | 4.201E−55 | SNRNP27 | 2.43E−44 | 0.264 | 0.19 | 0.29 | 2.807E−40 |
| RAB30-AS1 | 5.79E−59 | −0.251 | 0.035 | 0.332 | 6.680E−55 | RBP7 | 2.57E−44 | 0.954 | 0.361 | 0.087 | 2.968E−40 |
| FAM84B | 6.81E−59 | −0.264 | 0.079 | 0.423 | 7.860E−55 | NHP2 | 2.59E−44 | 0.377 | 0.369 | 0.49 | 2.985E−40 |
| LSM2 | 1.15E−58 | −0.367 | 0.062 | 0.42 | 1.331E−54 | ATP5G1 | 2.72E−44 | 0.450 | 0.267 | 0.282 | 3.135E−40 |
| CANT1 | 1.35E−58 | −0.369 | 0.113 | 0.499 | 1.559E−54 | SNX3 | 4.29E−44 | 0.451 | 0.208 | 0.192 | 4.951E−40 |
| HERPUD1 | 1.78E−58 | −0.330 | 0.068 | 0.411 | 2.051E−54 | SENP6 | 4.43E−44 | 0.301 | 0.203 | 0.338 | 5.116E−40 |
| SCD | 1.87E−58 | −0.741 | 0.246 | 0.67 | 2.159E−54 | HINT1 | 5.02E−44 | 0.552 | 0.641 | 0.715 | 5.791E−40 |
| HSF1 | 1.92E−58 | −0.319 | 0.044 | 0.389 | 2.219E−54 | CEP350 | 5.45E−44 | 0.604 | 0.307 | 0.313 | 6.295E−40 |
| TOB2 | 2.36E−58 | −0.342 | 0.048 | 0.397 | 2.729E−54 | ADAR | 5.89E−44 | 0.533 | 0.323 | 0.327 | 6.800E−40 |
| C1orf64 | 2.53E−58 | −0.300 | 0 | 0.273 | 2.920E−54 | MRP63 | 6.69E−44 | 0.438 | 0.201 | 0.203 | 7.716E−40 |
| MLEC | 3.52E−58 | −0.266 | 0.162 | 0.546 | 4.059E−54 | TCEAL8 | 6.83E−44 | 0.485 | 0.329 | 0.377 | 7.887E−40 |
| PFDN5 | 4.19E−58 | −0.493 | 0.308 | 0.786 | 4.833E−54 | GMPS | 7.72E−44 | 0.678 | 0.295 | 0.237 | 8.904E−40 |
| GLUD1 | 5.02E−58 | −0.292 | 0.061 | 0.4 | 5.798E−54 | ROCK1 | 8.57E−44 | 0.260 | 0.198 | 0.335 | 9.893E−40 |
| P4HA1 | 6.32E−58 | −0.409 | 0.11 | 0.496 | 7.288E−54 | DNAJC19 | 9.12E−44 | 0.433 | 0.254 | 0.282 | 1.052E−39 |
| FARSA | 1.08E−57 | −0.256 | 0.015 | 0.31 | 1.250E−53 | CARHSP1 | 1.09E−43 | 0.482 | 0.233 | 0.206 | 1.255E−39 |
| COMMD3 | 1.10E−57 | −0.294 | 0.043 | 0.366 | 1.271E−53 | TXNL1 | 1.21E−43 | 0.440 | 0.224 | 0.259 | 1.398E−39 |
| TSKU | 2.34E−57 | −0.278 | 0.004 | 0.285 | 2.698E−53 | HTATIP2 | 1.75E−43 | 0.576 | 0.251 | 0.172 | 2.023E−39 |
| ABCC11 | 4.51E−57 | −0.370 | 0.003 | 0.29 | 5.210E−53 | PSMC3 | 1.78E−43 | 0.363 | 0.242 | 0.33 | 2.052E−39 |
| MTHFD2 | 5.21E−57 | −0.352 | 0.213 | 0.62 | 6.008E−53 | PDCD5 | 1.92E−43 | 0.548 | 0.28 | 0.245 | 2.219E−39 |
| C4orf3 | 5.53E−57 | −0.398 | 0.207 | 0.625 | 6.380E−53 | DEGS1 | 1.94E−43 | 0.649 | 0.312 | 0.2 | 2.244E−39 |
| ATP5G2 | 5.62E−57 | −0.416 | 0.218 | 0.639 | 6.481E−53 | LAPTM4B | 2.06E−43 | 0.368 | 0.254 | 0.341 | 2.376E−39 |
| TMEM14C | 5.97E−57 | −0.574 | 0.234 | 0.71 | 6.889E−53 | TXNDC12 | 2.69E−43 | 0.282 | 0.16 | 0.256 | 3.104E−39 |
| CDH1 | 9.22E−57 | −0.342 | 0.029 | 0.349 | 1.065E−52 | PSMA3 | 3.51E−43 | 0.327 | 0.225 | 0.33 | 4.053E−39 |
| TNPO2 | 1.53E−56 | −0.345 | 0.031 | 0.361 | 1.761E−52 | UFM1 | 3.99E−43 | 0.497 | 0.26 | 0.265 | 4.604E−39 |
| NRBF2 | 2.31E−56 | −0.307 | 0.066 | 0.406 | 2.662E−52 | FAM50A | 4.06E−43 | 0.308 | 0.167 | 0.248 | 4.684E−39 |
| CLCN3 | 3.04E−56 | −0.305 | 0.07 | 0.411 | 3.509E−52 | PTPN11 | 4.64E−43 | 0.321 | 0.22 | 0.338 | 5.353E−39 |
| FADS2 | 3.39E−56 | −0.554 | 0.176 | 0.597 | 3.918E−52 | SNHG5 | 5.13E−43 | 0.441 | 0.391 | 0.468 | 5.918E−39 |
| CAMK2G | 3.94E−56 | −0.285 | 0.017 | 0.313 | 4.551E−52 | HLA-B | 5.18E−43 | 0.561 | 0.642 | 0.623 | 5.976E−39 |
| ZFAS1 | 9.38E−56 | −0.785 | 0.354 | 0.82 | 1.083E−51 | ELF3 | 5.91E−43 | 0.667 | 0.387 | 0.296 | 6.822E−39 |
| ARRDC1 | 1.46E−55 | −0.315 | 0.048 | 0.383 | 1.687E−51 | IFIH1 | 7.11E−43 | 0.931 | 0.32 | 0.076 | 8.202E−39 |
| CCDC174 | 1.52E−55 | −0.293 | 0.04 | 0.346 | 1.757E−51 | HSP90B1 | 7.68E−43 | 0.454 | 0.655 | 0.73 | 8.861E−39 |
| ORAOV1 | 2.63E−55 | −0.298 | 0.006 | 0.285 | 3.037E−51 | MRPL20 | 7.87E−43 | 0.366 | 0.277 | 0.358 | 9.079E−39 |
| ARFIP2 | 2.80E−55 | −0.267 | 0.009 | 0.287 | 3.234E−51 | GNL3 | 9.22E−43 | 0.369 | 0.314 | 0.423 | 1.064E−38 |
| GSE1 | 3.07E−55 | −0.339 | 0.013 | 0.313 | 3.545E−51 | ACBD6 | 1.01E−42 | 0.648 | 0.25 | 0.192 | 1.162E−38 |
| GS1-251I9.4 | 3.13E−55 | −0.295 | 0.025 | 0.33 | 3.615E−51 | EIF5 | 1.37E−42 | 0.256 | 0.319 | 0.473 | 1.585E−38 |
| AGR3 | 3.44E−55 | −0.337 | 0.005 | 0.282 | 3.971E−51 | CCL28 | 1.64E−42 | 0.916 | 0.304 | 0.039 | 1.895E−38 |
| MAFB | 5.63E−55 | −0.304 | 0.024 | 0.318 | 6.499E−51 | MPC2 | 2.07E−42 | 0.361 | 0.202 | 0.245 | 2.388E−38 |
| PABPC4 | 8.30E−55 | −0.271 | 0.076 | 0.403 | 9.582E−51 | CCT4 | 2.11E−42 | 0.278 | 0.303 | 0.434 | 2.441E−38 |
| DHTKD1 | 9.35E−55 | −0.279 | 0.065 | 0.389 | 1.079E−50 | CCDC109B | 2.48E−42 | 0.695 | 0.266 | 0.082 | 2.865E−38 |
| S100A16 | 1.19E−54 | −0.298 | 0.176 | 0.546 | 1.371E−50 | CIB1 | 2.52E−42 | 0.290 | 0.171 | 0.239 | 2.905E−38 |
| SLC2A10 | 1.61E−54 | −0.353 | 0.009 | 0.299 | 1.859E−50 | KPNA4 | 2.87E−42 | 0.492 | 0.241 | 0.225 | 3.310E−38 |
| RABEP1 | 1.65E−54 | −0.261 | 0.066 | 0.386 | 1.903E−50 | NASP | 2.93E−42 | 0.538 | 0.297 | 0.29 | 3.386E−38 |
| GUCY1A2 | 2.14E−54 | −0.315 | 0 | 0.256 | 2.471E−50 | ELOVL6 | 3.02E−42 | 0.867 | 0.301 | 0.045 | 3.483E−38 |
| TMEM86A | 2.14E−54 | −0.316 | 0 | 0.256 | 2.471E−50 | UHMK1 | 3.16E−42 | 0.482 | 0.197 | 0.197 | 3.642E−38 |
| MT1G | 2.14E−54 | −0.330 | 0 | 0.256 | 2.471E−50 | TMPO | 3.18E−42 | 0.687 | 0.265 | 0.138 | 3.674E−38 |
| CRAT | 2.22E−54 | −0.282 | 0.014 | 0.301 | 2.559E−50 | C1GALT1 | 3.90E−42 | 0.544 | 0.186 | 0.135 | 4.497E−38 |
| RPS27A | 2.32E−54 | −0.786 | 0.879 | 0.963 | 2.675E−50 | DEK | 4.95E−42 | 0.460 | 0.455 | 0.521 | 5.710E−38 |
| CD9 | 3.92E−54 | −0.332 | 0.282 | 0.704 | 4.521E−50 | COA6 | 4.95E−42 | 0.562 | 0.235 | 0.172 | 5.716E−38 |
| RNY1 | 4.38E−54 | −0.283 | 0.003 | 0.276 | 5.057E−50 | ACSL3 | 5.29E−42 | 0.432 | 0.292 | 0.318 | 6.106E−38 |
| EEF1B2 | 5.03E−54 | −0.751 | 0.393 | 0.834 | 5.803E−50 | TMSB15A | 5.63E−42 | 0.916 | 0.294 | 0.011 | 6.498E−38 |
| SLC2A4RG | 1.12E−53 | −0.292 | 0.028 | 0.315 | 1.296E−49 | VTCN1 | 6.18E−42 | 0.747 | 0.312 | 0.158 | 7.127E−38 |
| SPINT2 | 1.28E−53 | −0.469 | 0.268 | 0.715 | 1.482E−49 | ZMPSTE24 | 6.46E−42 | 0.351 | 0.185 | 0.237 | 7.456E−38 |
| RTKN2 | 1.29E−53 | −0.523 | 0.038 | 0.366 | 1.490E−49 | RNF213 | 7.43E−42 | 0.618 | 0.365 | 0.318 | 8.571E−38 |
| RPL9 | 1.40E−53 | −0.378 | 0.279 | 0.693 | 1.618E−49 | RSRC1 | 8.91E−42 | 0.301 | 0.214 | 0.366 | 1.029E−37 |
| RDX | 1.66E−53 | −0.278 | 0.076 | 0.397 | 1.918E−49 | PPP1R7 | 1.02E−41 | 0.371 | 0.214 | 0.268 | 1.176E−37 |
| C17orf76- | 1.69E−53 | −0.537 | 0.29 | 0.749 | 1.946E−49 | PAK2 | 1.11E−41 | 0.296 | 0.219 | 0.304 | 1.278E−37 |
| AS1 | | | | | | | | | | | |
| DLGAP1-AS1 | 3.13E−53 | −0.264 | 0.016 | 0.296 | 3.618E−49 | CLIC1 | 1.29E−41 | 0.275 | 0.161 | 0.223 | 1.488E−37 |
| MBOAT2 | 8.93E−53 | −0.300 | 0.029 | 0.33 | 1.031E−48 | EIF3I | 1.34E−41 | 0.336 | 0.24 | 0.307 | 1.550E−37 |
| GALNT6 | 1.90E−52 | −0.301 | 0.003 | 0.262 | 2.195E−48 | EHF | 1.47E−41 | 0.437 | 0.228 | 0.251 | 1.698E−37 |
| USP6NL | 2.13E−52 | −0.276 | 0.079 | 0.408 | 2.454E−48 | UCHL5 | 1.94E−41 | 0.670 | 0.305 | 0.18 | 2.243E−37 |
| G6PD | 2.39E−52 | −0.291 | 0.028 | 0.327 | 2.753E−48 | S100A13 | 2.29E−41 | 0.762 | 0.29 | 0.141 | 2.644E−37 |
| RPL6 | 3.31E−52 | −0.489 | 0.318 | 0.777 | 3.821E−48 | IQGAP1 | 2.51E−41 | 0.517 | 0.646 | 0.724 | 2.893E−37 |
| SLC20A1 | 3.79E−52 | −0.315 | 0.034 | 0.318 | 4.377E−48 | MAPKAP1 | 4.55E−41 | 0.327 | 0.194 | 0.301 | 5.255E−37 |
| RPS15 | 5.94E−52 | −0.790 | 0.528 | 0.885 | 6.860E−48 | ACTL6A | 5.01E−41 | 0.486 | 0.229 | 0.214 | 5.777E−37 |
| SELK | 7.04E−52 | −0.300 | 0.105 | 0.448 | 8.128E−48 | SMEK2 | 5.49E−41 | 0.401 | 0.234 | 0.29 | 6.334E−37 |
| UGDH | 8.52E−52 | −0.273 | 0.067 | 0.38 | 9.834E−48 | VPS29 | 6.11E−41 | 0.309 | 0.196 | 0.265 | 7.057E−37 |
| ABCA5 | 8.58E−52 | −0.292 | 0.016 | 0.293 | 9.905E−48 | MTIF3 | 6.25E−41 | 0.648 | 0.269 | 0.177 | 7.213E−37 |
| EIF5A | 1.03E−51 | −0.326 | 0.084 | 0.428 | 1.189E−47 | WFDC2 | 7.04E−41 | 0.847 | 0.31 | 0.042 | 8.129E−37 |
| HOOK2 | 1.21E−51 | −0.293 | 0.013 | 0.282 | 1.393E−47 | DST | 1.04E−40 | 0.468 | 0.333 | 0.42 | 1.195E−36 |
| TUBB | 2.57E−51 | −0.617 | 0.216 | 0.637 | 2.971E−47 | NAP1L4 | 1.10E−40 | 0.462 | 0.275 | 0.33 | 1.275E−36 |
| ASNS | 3.45E−51 | −0.325 | 0.027 | 0.318 | 3.979E−47 | GALNT1 | 1.36E−40 | 0.313 | 0.212 | 0.318 | 1.575E−36 |
| SMARCC1 | 4.13E−51 | −0.301 | 0.156 | 0.518 | 4.762E−47 | DNAJC3 | 1.49E−40 | 0.562 | 0.264 | 0.237 | 1.723E−36 |
| UBE2Q2 | 5.65E−51 | −0.272 | 0.029 | 0.313 | 6.524E−47 | LTF | 1.81E−40 | 0.947 | 0.284 | 0.011 | 2.086E−36 |
| PEBP1 | 6.42E−51 | −0.676 | 0.371 | 0.825 | 7.413E−47 | MDK | 2.23E−40 | 0.523 | 0.594 | 0.625 | 2.576E−36 |
| EPB41L4A | 7.27E−51 | −0.332 | 0.004 | 0.268 | 8.393E−47 | PRC1 | 2.28E−40 | 0.932 | 0.331 | 0.113 | 2.635E−36 |
| CCAR1 | 7.51E−51 | −0.257 | 0.087 | 0.408 | 8.669E−47 | S100A11 | 2.98E−40 | 0.377 | 0.614 | 0.744 | 3.436E−36 |
| H1F0 | 9.17E−51 | −0.313 | 0.167 | 0.513 | 1.059E−46 | PEG10 | 2.99E−40 | 0.793 | 0.627 | 0.451 | 3.450E−36 |
| RPL22 | 1.20E−50 | −0.595 | 0.304 | 0.758 | 1.386E−46 | ARID4B | 3.24E−40 | 0.391 | 0.211 | 0.254 | 3.744E−36 |
| ITPR1 | 1.93E−50 | −0.294 | 0.011 | 0.282 | 2.232E−46 | ZC3H11A | 4.45E−40 | 0.437 | 0.218 | 0.268 | 5.139E−36 |
| TRIB2 | 2.06E−50 | −0.259 | 0.089 | 0.403 | 2.379E−46 | PFDN2 | 5.37E−40 | 0.546 | 0.223 | 0.155 | 6.194E−36 |
| COX6C | 2.54E−50 | −0.399 | 0.358 | 0.794 | 2.929E−46 | SCCPDH | 5.51E−40 | 0.671 | 0.259 | 0.104 | 6.354E−36 |
| OAZ2 | 4.89E−50 | −0.289 | 0.037 | 0.335 | 5.639E−46 | LINC00998 | 5.78E−40 | 0.258 | 0.13 | 0.186 | 6.676E−36 |
| EIF5B | 7.26E−50 | −0.268 | 0.206 | 0.538 | 8.377E−46 | EDEM3 | 7.71E−40 | 0.646 | 0.21 | 0.079 | 8.895E−36 |
| HERC4 | 8.19E−50 | −0.267 | 0.043 | 0.324 | 9.447E−46 | SEC63 | 8.67E−40 | 0.276 | 0.201 | 0.321 | 1.001E−35 |
| PRPF6 | 8.32E−50 | −0.392 | 0.05 | 0.358 | 9.599E−46 | HN1 | 9.44E−40 | 0.651 | 0.293 | 0.22 | 1.090E−35 |
| PSAP | 1.02E−49 | −0.333 | 0.339 | 0.758 | 1.176E−45 | HUWE1 | 9.51E−40 | 0.311 | 0.194 | 0.287 | 1.098E−35 |
| BRD2 | 1.05E−49 | −0.299 | 0.166 | 0.532 | 1.211E−45 | PSME4 | 1.02E−39 | 0.700 | 0.32 | 0.237 | 1.172E−35 |
| RNF187 | 1.42E−49 | −0.261 | 0.05 | 0.33 | 1.644E−45 | POLR2B | 1.08E−39 | 0.355 | 0.198 | 0.248 | 1.250E−35 |
| PHEX | 2.83E−49 | −0.345 | 0.005 | 0.254 | 3.266E−45 | HADH | 1.11E−39 | 0.282 | 0.152 | 0.189 | 1.286E−35 |
| SAPCD2 | 3.83E−49 | −0.293 | 0.028 | 0.315 | 4.417E−45 | RWDD1 | 1.23E−39 | 0.382 | 0.247 | 0.301 | 1.418E−35 |
| RPLP2 | 4.05E−49 | −0.731 | 0.835 | 0.963 | 4.675E−45 | PLP2 | 1.46E−39 | 0.365 | 0.174 | 0.183 | 1.685E−35 |
| BTG1 | 4.33E−49 | −0.273 | 0.064 | 0.366 | 5.000E−45 | ACBD3 | 1.72E−39 | 0.403 | 0.211 | 0.237 | 1.989E−35 |
| ODF2L | 5.35E−49 | −0.251 | 0.052 | 0.33 | 6.180E−45 | UQCRFS1 | 3.03E−39 | 0.372 | 0.175 | 0.194 | 3.503E−35 |
| LRIG1 | 5.38E−49 | −0.314 | 0.016 | 0.29 | 6.211E−45 | TMEM9 | 3.55E−39 | 0.358 | 0.221 | 0.268 | 4.095E−35 |
| CYB5A | 6.91E−49 | −0.271 | 0.209 | 0.552 | 7.977E−45 | COPS2 | 3.57E−39 | 0.401 | 0.214 | 0.282 | 4.115E−35 |
| MRFAP1L1 | 7.72E−49 | −0.253 | 0.044 | 0.332 | 8.906E−45 | NUCB2 | 3.61E−39 | 0.379 | 0.272 | 0.375 | 4.168E−35 |
| ARID5B | 8.52E−49 | −0.261 | 0.09 | 0.411 | 9.832E−45 | EBNA1BP2 | 3.66E−39 | 0.461 | 0.21 | 0.208 | 4.226E−35 |
| TRPS1 | 8.58E−49 | −0.288 | 0.223 | 0.572 | 9.899E−45 | ARMCX3 | 3.77E−39 | 0.707 | 0.286 | 0.13 | 4.347E−35 |
| PRPF4B | 1.29E−48 | −0.272 | 0.137 | 0.482 | 1.484E−44 | POLE3 | 3.94E−39 | 0.261 | 0.19 | 0.304 | 4.545E−35 |
| TFF3 | 1.36E−48 | −0.361 | 0 | 0.231 | 1.572E−44 | NGFRAP1 | 4.63E−39 | 0.412 | 0.223 | 0.259 | 5.344E−35 |
| AP3B1 | 1.55E−48 | −0.251 | 0.028 | 0.293 | 1.792E−44 | SMIM22 | 5.13E−39 | 0.508 | 0.269 | 0.214 | 5.918E−35 |
| SLC1A4 | 2.08E−48 | −0.289 | 0.021 | 0.301 | 2.397E−44 | SRSF7 | 7.17E−39 | 0.365 | 0.206 | 0.231 | 8.270E−35 |
| PPP3CA | 2.64E−48 | −0.447 | 0.076 | 0.417 | 3.052E−44 | ASH1L | 7.47E−39 | 0.374 | 0.2 | 0.248 | 8.619E−35 |
| EGR2 | 3.43E−48 | −0.294 | 0.001 | 0.239 | 3.954E−44 | SMC4 | 7.58E−39 | 0.757 | 0.356 | 0.237 | 8.743E−35 |
| RPS27 | 4.03E−48 | −0.631 | 0.391 | 0.811 | 4.656E−44 | RALA | 7.68E−39 | 0.689 | 0.299 | 0.18 | 8.865E−35 |
| CLDN3 | 2.13E−47 | −0.299 | 0.25 | 0.628 | 2.463E−43 | ABI1 | 9.81E−39 | 0.469 | 0.239 | 0.234 | 1.132E−34 |
| ZNF704 | 2.61E−47 | −0.312 | 0.022 | 0.293 | 3.017E−43 | GTPBP4 | 1.02E−38 | 0.492 | 0.228 | 0.228 | 1.175E−34 |
| IRX2 | 4.10E−47 | −0.261 | 0.013 | 0.265 | 4.729E−43 | PHF20L1 | 1.38E−38 | 0.352 | 0.249 | 0.332 | 1.591E−34 |
| ROMO1 | 6.29E−47 | −0.519 | 0.324 | 0.763 | 7.259E−43 | PRMT2 | 1.55E−38 | 0.268 | 0.234 | 0.358 | 1.788E−34 |
| ZFYVE20 | 7.01E−47 | −0.275 | 0.018 | 0.282 | 8.086E−43 | PFN2 | 1.65E−38 | 0.256 | 0.16 | 0.242 | 1.905E−34 |
| GABRQ | 1.11E−46 | −0.397 | 0 | 0.223 | 1.283E−42 | IFITM1 | 1.67E−38 | 0.810 | 0.3 | 0.023 | 1.927E−34 |
| RPSA | 1.29E−46 | −0.341 | 0.33 | 0.744 | 1.487E−42 | GUCY1A3 | 1.69E−38 | 0.836 | 0.277 | 0.003 | 1.952E−34 |
| CLU | 1.80E−46 | −0.313 | 0.188 | 0.544 | 2.081E−42 | NUDCD1 | 1.83E−38 | 0.417 | 0.234 | 0.268 | 2.110E−34 |
| AC104667.3 | 2.04E−46 | −0.289 | 0.009 | 0.242 | 2.350E−42 | BZW2 | 1.91E−38 | 0.307 | 0.183 | 0.251 | 2.206E−34 |
| JUN | 3.22E−46 | −0.561 | 0.299 | 0.704 | 3.714E−42 | VMA21 | 2.42E−38 | 0.289 | 0.187 | 0.259 | 2.789E−34 |
| RNMT | 3.87E−46 | −0.255 | 0.065 | 0.358 | 4.469E−42 | PSMD12 | 2.90E−38 | 0.580 | 0.248 | 0.214 | 3.344E−34 |
| CALR | 3.99E−46 | −0.735 | 0.474 | 0.859 | 4.601E−42 | RER1 | 3.24E−38 | 0.260 | 0.148 | 0.197 | 3.736E−34 |
| SCGB2A1 | 1.03E−45 | −0.269 | 0.012 | 0.262 | 1.184E−41 | MBNL2 | 3.28E−38 | 0.563 | 0.276 | 0.242 | 3.781E−34 |
| NR4A1 | 1.59E−45 | −0.396 | 0.016 | 0.285 | 1.840E−41 | SCRN1 | 3.31E−38 | 0.465 | 0.232 | 0.225 | 3.818E−34 |
| SYCP2 | 1.66E−45 | −0.321 | 0.087 | 0.392 | 1.919E−41 | ST3GAL1 | 3.59E−38 | 0.270 | 0.169 | 0.254 | 4.138E−34 |
| TNFRSF11B | 4.86E−45 | −0.360 | 0.003 | 0.234 | 5.605E−41 | CD44 | 3.93E−38 | 0.721 | 0.324 | 0.155 | 4.533E−34 |
| SIGLEC15 | 8.80E−45 | −0.264 | 0.002 | 0.228 | 1.016E−40 | DAP3 | 5.24E−38 | 0.406 | 0.202 | 0.208 | 6.053E−34 |
| RP13- | 1.14E−44 | −0.322 | 0.004 | 0.234 | 1.316E−40 | CD55 | 5.64E−38 | 0.696 | 0.363 | 0.214 | 6.514E−34 |
| 895J2.7 | | | | | | | | | | | |
| NR4A2 | 1.88E−44 | −0.276 | 0.002 | 0.22 | 2.171E−40 | CREG1 | 5.91E−38 | 0.284 | 0.165 | 0.223 | 6.819E−34 |
| DNAJC1 | 2.05E−44 | −0.352 | 0.128 | 0.454 | 2.371E−40 | PSMB3 | 7.86E−38 | 0.357 | 0.211 | 0.234 | 9.077E−34 |
| CHD6 | 5.29E−44 | −0.319 | 0.021 | 0.282 | 6.103E−40 | RNF13 | 8.52E−38 | 0.260 | 0.158 | 0.225 | 9.834E−34 |
| ARIH1 | 5.94E−44 | −0.267 | 0.016 | 0.254 | 6.861E−40 | CKAP4 | 1.04E−37 | 0.292 | 0.154 | 0.206 | 1.198E−33 |
| TAF1D | 8.10E−44 | −0.263 | 0.077 | 0.363 | 9.347E−40 | TIMM17A | 1.06E−37 | 0.541 | 0.241 | 0.169 | 1.228E−33 |
| RUFY2 | 1.26E−43 | −0.321 | 0.027 | 0.287 | 1.455E−39 | FARP1 | 1.17E−37 | 0.520 | 0.245 | 0.18 | 1.351E−33 |
| EPAS1 | 1.42E−43 | −0.270 | 0.008 | 0.245 | 1.634E−39 | TIPRL | 1.39E−37 | 0.625 | 0.245 | 0.155 | 1.604E−33 |
| CD36 | 1.76E−43 | −0.372 | 0.003 | 0.22 | 2.034E−39 | COPA | 1.79E−37 | 0.333 | 0.193 | 0.259 | 2.065E−33 |
| ADO | 3.01E−43 | −0.260 | 0.016 | 0.262 | 3.479E−39 | TP53BP2 | 2.26E−37 | 0.576 | 0.219 | 0.113 | 2.606E−33 |
| H1FX | 5.24E−43 | −0.257 | 0.043 | 0.31 | 6.048E−39 | DUT | 2.41E−37 | 0.452 | 0.258 | 0.239 | 2.777E−33 |
| GANAB | 5.41E−43 | −0.251 | 0.037 | 0.296 | 6.248E−39 | EIF2A | 2.46E−37 | 0.377 | 0.199 | 0.231 | 2.844E−33 |
| GADD45B | 8.79E−43 | −0.325 | 0.067 | 0.346 | 1.014E−38 | IFI16 | 2.69E−37 | 0.887 | 0.328 | 0.042 | 3.106E−33 |
| WWC2 | 3.74E−42 | −0.285 | 0.002 | 0.211 | 4.316E−38 | HSPH1 | 2.70E−37 | 0.538 | 0.307 | 0.287 | 3.116E−33 |
| UNC5B | 7.37E−42 | −0.265 | 0.002 | 0.208 | 8.503E−38 | IRAK4 | 3.02E−37 | 0.630 | 0.256 | 0.138 | 3.491E−33 |
| RPS20 | 9.99E−42 | −0.658 | 0.591 | 0.91 | 1.153E−37 | SP100 | 3.11E−37 | 0.549 | 0.254 | 0.217 | 3.585E−33 |
| LSM14B | 1.25E−41 | −0.253 | 0.015 | 0.254 | 1.445E−37 | BTG3 | 3.49E−37 | 0.843 | 0.285 | 0.054 | 4.024E−33 |
| FBXO32 | 2.54E−40 | −0.350 | 0.019 | 0.259 | 2.928E−36 | PCNP | 3.82E−37 | 0.341 | 0.245 | 0.315 | 4.404E−33 |
| EEF1A1 | 2.73E−40 | −0.711 | 0.59 | 0.868 | 3.145E−36 | RGS2 | 3.85E−37 | 0.881 | 0.366 | 0.175 | 4.444E−33 |
| GOLGA4 | 3.59E−40 | −0.273 | 0.304 | 0.648 | 4.145E−36 | ATAD2 | 4.28E−37 | 0.500 | 0.203 | 0.18 | 4.936E−33 |
| RNASEH2A | 3.73E−40 | −0.274 | 0.029 | 0.279 | 4.307E−36 | DDX18 | 5.35E−37 | 0.256 | 0.189 | 0.301 | 6.175E−33 |
| PKP2 | 3.63E−39 | −0.273 | 0.024 | 0.259 | 4.186E−35 | CCT8 | 6.15E−37 | 0.290 | 0.148 | 0.183 | 7.096E−33 |
| RPL10 | 6.04E−39 | −0.615 | 0.456 | 0.837 | 6.970E−35 | ANLN | 6.53E−37 | 0.654 | 0.224 | 0.158 | 7.537E−33 |
| MT-TY | 9.59E−39 | −0.265 | 0.004 | 0.206 | 1.107E−34 | RAI14 | 6.68E−37 | 0.850 | 0.3 | 0.051 | 7.706E−33 |
| ERRFI1 | 1.70E−38 | −0.282 | 0.095 | 0.355 | 1.964E−34 | PPHLN1 | 7.45E−37 | 0.429 | 0.177 | 0.149 | 8.601E−33 |
| CCDC91 | 2.00E−38 | −0.267 | 0.056 | 0.318 | 2.306E−34 | SYNE2 | 8.71E−37 | 0.543 | 0.426 | 0.434 | 1.005E−32 |
| ERMN | 2.12E−38 | −0.256 | 0.001 | 0.194 | 2.452E−34 | IRF2BP2 | 9.06E−37 | 0.596 | 0.284 | 0.189 | 1.045E−32 |
| RPS5 | 2.54E−38 | −0.563 | 0.792 | 0.949 | 2.928E−34 | LIMCH1 | 1.00E−36 | 0.302 | 0.275 | 0.372 | 1.157E−32 |
| MGEA5 | 3.12E−38 | −0.256 | 0.094 | 0.361 | 3.602E−34 | BBOX1 | 1.21E−36 | 0.783 | 0.265 | 0.003 | 1.396E−32 |
| FUT11 | 1.16E−37 | −0.305 | 0.022 | 0.256 | 1.335E−33 | OSTC | 1.45E−36 | 0.417 | 0.281 | 0.327 | 1.678E−32 |
| RP11- | 1.95E−37 | −0.250 | 0.036 | 0.27 | 2.251E−33 | VAMP7 | 1.98E−36 | 0.361 | 0.154 | 0.169 | 2.288E−32 |
| 22011.1 | | | | | | | | | | | |
| HIST1H4H | 4.30E−37 | −0.284 | 0.031 | 0.256 | 4.964E−33 | HMGN3 | 2.00E−36 | 0.448 | 0.205 | 0.177 | 2.313E−32 |
| RPL3 | 6.82E−36 | −0.582 | 0.513 | 0.862 | 7.871E−32 | NFIB | 2.50E−36 | 0.666 | 0.301 | 0.161 | 2.880E−32 |
| TK1 | 3.52E−35 | −0.480 | 0.011 | 0.225 | 4.057E−31 | TC2N | 3.37E−36 | 0.445 | 0.21 | 0.211 | 3.887E−32 |
| SLFN13 | 5.13E−34 | −0.294 | 0.05 | 0.285 | 5.925E−30 | MCM3 | 3.89E−36 | 0.347 | 0.189 | 0.228 | 4.486E−32 |
| EEF2 | 6.24E−34 | −0.283 | 0.453 | 0.808 | 7.205E−30 | MT-ND6 | 4.26E−36 | 0.346 | 0.265 | 0.355 | 4.916E−32 |
| RPL36 | 1.13E−33 | −0.431 | 0.549 | 0.896 | 1.307E−29 | PPP1R2 | 4.30E−36 | 0.435 | 0.175 | 0.175 | 4.957E−32 |
| ZNF365 | 1.32E−33 | −0.259 | 0 | 0.163 | 1.522E−29 | IRAK1 | 4.49E−36 | 0.545 | 0.267 | 0.144 | 5.178E−32 |
| HSPA5 | 9.25E−33 | −0.263 | 0.432 | 0.738 | 1.068E−28 | FAM49B | 5.23E−36 | 0.418 | 0.183 | 0.169 | 6.035E−32 |
| DSP | 2.13E−32 | −0.318 | 0.531 | 0.848 | 2.463E−28 | RRP15 | 5.48E−36 | 0.378 | 0.175 | 0.158 | 6.323E−32 |
| MT-TH | 5.69E−32 | −0.357 | 0.008 | 0.194 | 6.569E−28 | EIF2S1 | 5.85E−36 | 0.280 | 0.167 | 0.259 | 6.749E−32 |
| RPL11 | 2.85E−30 | −0.444 | 0.865 | 0.977 | 3.289E−26 | RPL23 | 6.32E−36 | 0.486 | 0.709 | 0.783 | 7.300E−32 |
| MALAT1 | 3.41E−30 | −1.216 | 0.929 | 0.98 | 3.940E−26 | FLNA | 6.60E−36 | 0.414 | 0.21 | 0.169 | 7.620E−32 |
| ACTG1 | 4.87E−30 | −0.565 | 0.756 | 0.949 | 5.616E−26 | POLR1D | 7.53E−36 | 0.561 | 0.244 | 0.186 | 8.689E−32 |
| MT-TM | 7.29E−30 | −0.253 | 0.004 | 0.166 | 8.418E−26 | MACF1 | 7.99E−36 | 0.415 | 0.197 | 0.223 | 9.225E−32 |
| IGFBP5 | 4.14E−29 | −0.496 | 0.054 | 0.279 | 4.784E−25 | HIST1H4C | 9.18E−36 | 0.383 | 0.62 | 0.608 | 1.059E−31 |
| SORL1 | 6.23E−29 | −0.883 | 0.018 | 0.197 | 7.185E−25 | MED10 | 1.38E−35 | 0.549 | 0.205 | 0.087 | 1.589E−31 |
| RPL4 | 2.16E−28 | −0.468 | 0.656 | 0.921 | 2.498E−24 | TAPBP | 1.42E−35 | 0.366 | 0.223 | 0.239 | 1.636E−31 |
| RPL13A | 2.42E−28 | −0.544 | 0.805 | 0.938 | 2.794E−24 | TALDO1 | 1.97E−35 | 0.270 | 0.154 | 0.206 | 2.268E−31 |
| EGLN3 | 1.90E−24 | −0.266 | 0.032 | 0.211 | 2.188E−20 | LPGAT1 | 2.14E−35 | 0.667 | 0.305 | 0.186 | 2.465E−31 |
| FTL | 2.78E−24 | −0.375 | 0.611 | 0.848 | 3.211E−20 | ABRACL | 2.38E−35 | 0.591 | 0.274 | 0.166 | 2.751E−31 |
| RPS4X | 1.17E−23 | −0.488 | 0.637 | 0.868 | 1.352E−19 | ATP1B1 | 2.76E−35 | 0.323 | 0.276 | 0.33 | 3.180E−31 |
| CDK1 | 5.13E−23 | −0.320 | 0.116 | 0.301 | 5.925E−19 | LARP4B | 3.43E−35 | 0.346 | 0.15 | 0.149 | 3.956E−31 |
| CCL3 | 5.10E−22 | −0.357 | 0.001 | 0.118 | 5.881E−18 | SFT2D2 | 4.37E−35 | 0.556 | 0.216 | 0.082 | 5.048E−31 |
| RPL10A | 2.64E−21 | −0.398 | 0.601 | 0.865 | 3.043E−17 | IFNGR1 | 5.09E−35 | 0.280 | 0.173 | 0.228 | 5.870E−31 |
| UBB | 8.78E−21 | −0.363 | 0.587 | 0.854 | 1.013E−16 | SLC25A36 | 5.57E−35 | 0.269 | 0.146 | 0.211 | 6.433E−31 |
| MNDA | 8.85E−21 | −0.284 | 0.019 | 0.138 | 1.021E−16 | MBNL1 | 5.84E−35 | 0.524 | 0.237 | 0.18 | 6.745E−31 |
| RPS12 | 3.43E−19 | −0.364 | 0.979 | 0.994 | 3.956E−15 | KRTCAP3 | 6.41E−35 | 0.258 | 0.109 | 0.113 | 7.403E−31 |
| RPL12 | 5.19E−19 | −0.390 | 0.694 | 0.907 | 5.993E−15 | COX20 | 7.06E−35 | 0.605 | 0.264 | 0.183 | 8.150E−31 |
| RPL35 | 3.54E−18 | −0.358 | 0.922 | 0.975 | 4.081E−14 | TMX1 | 7.67E−35 | 0.382 | 0.152 | 0.11 | 8.857E−31 |
| H3F3B | 1.14E−17 | −0.338 | 0.715 | 0.921 | 1.312E−13 | EML4 | 9.14E−35 | 0.408 | 0.189 | 0.163 | 1.054E−30 |
| RPS29 | 2.02E−16 | −0.392 | 0.936 | 0.966 | 2.329E−12 | ANAPC11 | 1.13E−34 | 0.350 | 0.58 | 0.713 | 1.298E−30 |
| GNB2L1 | 7.11E−15 | −0.265 | 0.741 | 0.918 | 8.203E−11 | TNFSF10 | 1.19E−34 | 0.532 | 0.317 | 0.299 | 1.370E−30 |
| RPL35A | 1.20E−14 | −0.373 | 0.934 | 0.983 | 1.385E−10 | CMSS1 | 1.55E−34 | 0.420 | 0.181 | 0.214 | 1.785E−30 |
| | | | | | | CMTM6 | 1.57E−34 | 0.268 | 0.165 | 0.211 | 1.816E−30 |
| | | | | | | RNF7 | 1.65E−34 | 0.362 | 0.229 | 0.254 | 1.910E−30 |
| | | | | | | CLIP1 | 1.82E−34 | 0.410 | 0.294 | 0.315 | 2.101E−30 |
| | | | | | | MYL12A | 1.82E−34 | 0.331 | 0.565 | 0.69 | 2.104E−30 |
| | | | | | | CDC16 | 1.96E−34 | 0.364 | 0.181 | 0.183 | 2.258E−30 |
| | | | | | | STK24 | 2.04E−34 | 0.483 | 0.214 | 0.146 | 2.359E−30 |
| | | | | | | IPO5 | 2.15E−34 | 0.467 | 0.249 | 0.242 | 2.481E−30 |
| | | | | | | XIST | 2.28E−34 | 0.947 | 0.31 | 0.056 | 2.631E−30 |
| | | | | | | HIBADH | 2.29E−34 | 0.343 | 0.126 | 0.115 | 2.639E−30 |
| | | | | | | MAGOH | 3.16E−34 | 0.289 | 0.163 | 0.208 | 3.646E−30 |
| | | | | | | PDS5A | 3.70E−34 | 0.273 | 0.186 | 0.251 | 4.265E−30 |
| | | | | | | PAPSS1 | 3.91E−34 | 0.562 | 0.229 | 0.135 | 4.517E−30 |
| | | | | | | CENPF | 4.84E−34 | 1.103 | 0.466 | 0.231 | 5.586E−30 |
| | | | | | | ARHGAP5 | 4.90E−34 | 0.378 | 0.242 | 0.279 | 5.656E−30 |
| | | | | | | IFI27L2 | 4.99E−34 | 0.660 | 0.231 | 0.011 | 5.764E−30 |
| | | | | | | MRPL9 | 5.59E−34 | 0.561 | 0.196 | 0.085 | 6.453E−30 |
| | | | | | | APOD | 5.87E−34 | 0.748 | 0.324 | 0.121 | 6.770E−30 |
| | | | | | | ITGB1BP1 | 6.09E−34 | 0.267 | 0.122 | 0.146 | 7.025E−30 |
| | | | | | | GSN | 6.54E−34 | 0.275 | 0.194 | 0.237 | 7.551E−30 |
| | | | | | | LBR | 1.03E−33 | 0.804 | 0.305 | 0.13 | 1.190E−29 |
| | | | | | | CD2AP | 1.15E−33 | 0.436 | 0.214 | 0.245 | 1.325E−29 |
| | | | | | | ZFAND5 | 1.18E−33 | 0.318 | 0.175 | 0.211 | 1.361E−29 |
| | | | | | | CDC73 | 2.13E−33 | 0.418 | 0.198 | 0.192 | 2.453E−29 |
| | | | | | | SPTBN1 | 2.21E−33 | 0.324 | 0.201 | 0.248 | 2.548E−29 |
| | | | | | | SOX9 | 3.01E−33 | 0.561 | 0.251 | 0.152 | 3.473E−29 |
| | | | | | | ATF6 | 3.67E−33 | 0.284 | 0.209 | 0.285 | 4.237E−29 |
| | | | | | | LGALS3BP | 4.11E−33 | 0.674 | 0.247 | 0.031 | 4.741E−29 |
| | | | | | | IVNS1ABP | 4.30E−33 | 0.560 | 0.242 | 0.18 | 4.963E−29 |
| | | | | | | TES | 4.54E−33 | 0.379 | 0.205 | 0.242 | 5.239E−29 |
| | | | | | | OGFRL1 | 4.84E−33 | 0.600 | 0.237 | 0.141 | 5.586E−29 |
| | | | | | | C19orf70 | 5.31E−33 | 0.268 | 0.171 | 0.223 | 6.126E−29 |
| | | | | | | DESI2 | 5.36E−33 | 0.631 | 0.242 | 0.13 | 6.183E−29 |
| | | | | | | BROX | 5.79E−33 | 0.623 | 0.297 | 0.214 | 6.683E−29 |
| | | | | | | PALLD | 7.03E−33 | 0.531 | 0.252 | 0.135 | 8.112E−29 |
| | | | | | | SMC1A | 9.02E−33 | 0.544 | 0.218 | 0.166 | 1.041E−28 |
| | | | | | | MRPL47 | 9.42E−33 | 0.390 | 0.24 | 0.237 | 1.088E−28 |
| | | | | | | TMEM14A | 9.68E−33 | 0.586 | 0.221 | 0.07 | 1.117E−28 |
| | | | | | | PPP2R5C | 1.07E−32 | 0.344 | 0.157 | 0.175 | 1.239E−28 |
| | | | | | | ZHX1 | 1.12E−32 | 0.560 | 0.242 | 0.211 | 1.295E−28 |
| | | | | | | DCAF13 | 1.23E−32 | 0.324 | 0.15 | 0.163 | 1.424E−28 |
| | | | | | | HIPK3 | 1.26E−32 | 0.262 | 0.101 | 0.115 | 1.450E−28 |
| | | | | | | ALDH1A3 | 1.57E−32 | 0.593 | 0.223 | 0.115 | 1.811E−28 |
| | | | | | | NCOA7 | 1.57E−32 | 0.265 | 0.184 | 0.22 | 1.816E−28 |
| | | | | | | UBE2N | 1.59E−32 | 0.472 | 0.186 | 0.115 | 1.831E−28 |
| | | | | | | MTRNR2L6 | 1.70E−32 | 0.514 | 0.21 | 0.082 | 1.967E−28 |
| | | | | | | S100B | 1.75E−32 | 0.470 | 0.229 | 0.132 | 2.025E−28 |
| | | | | | | IL6ST | 2.15E−32 | 0.271 | 0.184 | 0.248 | 2.478E−28 |
| | | | | | | PLOD2 | 2.20E−32 | 0.587 | 0.234 | 0.107 | 2.541E−28 |
| | | | | | | LIMS1 | 2.79E−32 | 0.295 | 0.147 | 0.166 | 3.222E−28 |
| | | | | | | NAA38 | 2.93E−32 | 0.258 | 0.131 | 0.152 | 3.378E−28 |
| | | | | | | PSMC2 | 3.05E−32 | 0.262 | 0.124 | 0.166 | 3.517E−28 |
| | | | | | | TMEM126B | 3.34E−32 | 0.357 | 0.154 | 0.155 | 3.849E−28 |
| | | | | | | HAX1 | 5.74E−32 | 0.307 | 0.152 | 0.177 | 6.628E−28 |
| | | | | | | COTL1 | 5.99E−32 | 0.488 | 0.226 | 0.146 | 6.916E−28 |
| | | | | | | ICT1 | 7.51E−32 | 0.327 | 0.134 | 0.144 | 8.665E−28 |
| | | | | | | KRCC1 | 9.18E−32 | 0.401 | 0.167 | 0.132 | 1.060E−27 |
| | | | | | | PANK3 | 1.13E−31 | 0.283 | 0.169 | 0.211 | 1.305E−27 |
| | | | | | | MFF | 1.22E−31 | 0.272 | 0.152 | 0.189 | 1.404E−27 |
| | | | | | | ANAPC13 | 1.26E−31 | 0.270 | 0.114 | 0.107 | 1.452E−27 |
| | | | | | | NT5C3A | 1.37E−31 | 0.405 | 0.182 | 0.158 | 1.586E−27 |
| | | | | | | STK38 | 1.69E−31 | 0.262 | 0.111 | 0.118 | 1.947E−27 |
| | | | | | | PSMB8 | 1.76E−31 | 0.494 | 0.202 | 0.115 | 2.029E−27 |
| | | | | | | TSEN15 | 1.89E−31 | 0.465 | 0.184 | 0.107 | 2.183E−27 |
| | | | | | | PDLIM5 | 2.72E−31 | 0.528 | 0.227 | 0.152 | 3.134E−27 |
| | | | | | | SUCO | 3.49E−31 | 0.265 | 0.157 | 0.242 | 4.023E−27 |
| | | | | | | TMEM30A | 3.67E−31 | 0.348 | 0.207 | 0.273 | 4.238E−27 |
| | | | | | | ADSS | 4.00E−31 | 0.580 | 0.263 | 0.214 | 4.615E−27 |
| | | | | | | OPTN | 4.48E−31 | 0.747 | 0.262 | 0.014 | 5.170E−27 |
| | | | | | | COG2 | 4.68E−31 | 0.330 | 0.123 | 0.099 | 5.398E−27 |
| | | | | | | GNPTG | 6.34E−31 | 0.329 | 0.157 | 0.161 | 7.314E−27 |
| | | | | | | SUGT1 | 7.64E−31 | 0.695 | 0.259 | 0.085 | 8.820E−27 |
| | | | | | | MRPS14 | 7.65E−31 | 0.454 | 0.194 | 0.152 | 8.831E−27 |
| | | | | | | SMARCA2 | 8.46E−31 | 0.330 | 0.18 | 0.214 | 9.768E−27 |
| | | | | | | PARP9 | 9.21E−31 | 0.447 | 0.186 | 0.169 | 1.063E−26 |
| | | | | | | LCP1 | 1.17E−30 | 0.662 | 0.259 | 0.101 | 1.346E−26 |
| | | | | | | TPRKB | 1.41E−30 | 0.356 | 0.211 | 0.248 | 1.632E−26 |
| | | | | | | SRD5A3 | 1.98E−30 | 0.310 | 0.157 | 0.175 | 2.281E−26 |
| | | | | | | TOMM70A | 2.06E−30 | 0.303 | 0.172 | 0.234 | 2.381E−26 |
| | | | | | | NSMCE2 | 2.21E−30 | 0.461 | 0.167 | 0.146 | 2.549E−26 |
| | | | | | | MRPS15 | 2.28E−30 | 0.310 | 0.169 | 0.211 | 2.629E−26 |
| | | | | | | SNRPA1 | 2.29E−30 | 0.509 | 0.218 | 0.107 | 2.648E−26 |
| | | | | | | ITPR2 | 3.69E−30 | 0.312 | 0.159 | 0.225 | 4.259E−26 |
| | | | | | | SUN1 | 4.54E−30 | 0.460 | 0.188 | 0.107 | 5.234E−26 |
| | | | | | | LPP | 5.75E−30 | 0.383 | 0.187 | 0.158 | 6.639E−26 |
| | | | | | | PRPSAP1 | 1.01E−29 | 0.322 | 0.13 | 0.107 | 1.168E−25 |
| | | | | | | BRCC3 | 1.16E−29 | 0.391 | 0.175 | 0.183 | 1.336E−25 |
| | | | | | | MYH9 | 1.69E−29 | 0.304 | 0.223 | 0.279 | 1.947E−25 |
| | | | | | | CASP6 | 1.80E−29 | 0.279 | 0.129 | 0.149 | 2.082E−25 |
| | | | | | | IFNAR1 | 1.87E−29 | 0.561 | 0.206 | 0.135 | 2.159E−25 |
| | | | | | | PLRG1 | 2.04E−29 | 0.333 | 0.161 | 0.18 | 2.352E−25 |
| | | | | | | POGK | 2.11E−29 | 0.276 | 0.122 | 0.13 | 2.436E−25 |
| | | | | | | FMR1 | 2.67E−29 | 0.345 | 0.177 | 0.211 | 3.080E−25 |
| | | | | | | RAB4A | 3.37E−29 | 0.256 | 0.134 | 0.175 | 3.886E−25 |
| | | | | | | PPP2R5A | 3.46E−29 | 0.464 | 0.223 | 0.161 | 3.991E−25 |
| | | | | | | TUBA1A | 3.51E−29 | 0.279 | 0.177 | 0.192 | 4.049E−25 |
| | | | | | | MT-ND5 | 3.68E−29 | 0.370 | 0.59 | 0.707 | 4.247E−25 |
| | | | | | | ZBTB38 | 4.02E−29 | 0.503 | 0.201 | 0.177 | 4.636E−25 |
| | | | | | | H3F3A | 4.72E−29 | 0.372 | 0.144 | 0.085 | 5.447E−25 |
| | | | | | | OCIAD2 | 5.47E−29 | 0.375 | 0.208 | 0.2 | 6.310E−25 |
| | | | | | | SAP30BP | 7.39E−29 | 0.254 | 0.167 | 0.239 | 8.525E−25 |
| | | | | | | CDV3 | 8.53E−29 | 0.501 | 0.256 | 0.197 | 9.848E−25 |
| | | | | | | TMEM106B | 1.02E−28 | 0.332 | 0.158 | 0.152 | 1.177E−24 |
| | | | | | | TRMT10C | 1.14E−28 | 0.364 | 0.147 | 0.155 | 1.318E−24 |
| | | | | | | VWA1 | 1.28E−28 | 0.324 | 0.133 | 0.101 | 1.477E−24 |
| | | | | | | SPATS2L | 2.06E−28 | 0.489 | 0.209 | 0.161 | 2.378E−24 |
| | | | | | | ESD | 2.59E−28 | 0.388 | 0.17 | 0.135 | 2.988E−24 |
| | | | | | | TFDP1 | 2.64E−28 | 0.398 | 0.167 | 0.099 | 3.043E−24 |
| | | | | | | USP15 | 2.75E−28 | 0.318 | 0.128 | 0.146 | 3.168E−24 |
| | | | | | | UBE2J1 | 3.09E−28 | 0.440 | 0.185 | 0.127 | 3.572E−24 |
| | | | | | | ANP32E | 3.39E−28 | 0.619 | 0.196 | 0.054 | 3.911E−24 |
| | | | | | | LRRFIP2 | 3.70E−28 | 0.298 | 0.159 | 0.192 | 4.267E−24 |
| | | | | | | TSPAN6 | 4.04E−28 | 0.515 | 0.199 | 0.11 | 4.667E−24 |
| | | | | | | BEX2 | 4.65E−28 | 0.568 | 0.222 | 0.144 | 5.370E−24 |
| | | | | | | CDCA7L | 5.89E−28 | 0.477 | 0.206 | 0.127 | 6.798E−24 |
| | | | | | | MESP1 | 7.63E−28 | 0.530 | 0.173 | 0.048 | 8.806E−24 |
| | | | | | | SMC3 | 7.80E−28 | 0.253 | 0.206 | 0.287 | 9.003E−24 |
| | | | | | | SKIL | 8.13E−28 | 0.382 | 0.176 | 0.203 | 9.384E−24 |
| | | | | | | ACAT2 | 8.90E−28 | 0.282 | 0.13 | 0.132 | 1.027E−23 |
| | | | | | | LINC00152 | 1.16E−27 | 0.652 | 0.249 | 0.051 | 1.343E−23 |
| | | | | | | CEACAM6 | 1.21E−27 | 0.661 | 0.228 | 0.039 | 1.401E−23 |
| | | | | | | CTNNAL1 | 1.54E−27 | 0.422 | 0.133 | 0.056 | 1.775E−23 |
| | | | | | | HEXB | 1.88E−27 | 0.376 | 0.13 | 0.085 | 2.166E−23 |
| | | | | | | TXNIP | 2.09E−27 | 0.677 | 0.274 | 0.107 | 2.417E−23 |
| | | | | | | CDK16 | 2.72E−27 | 0.269 | 0.124 | 0.115 | 3.138E−23 |
| | | | | | | GTF2F2 | 2.75E−27 | 0.485 | 0.174 | 0.085 | 3.170E−23 |
| | | | | | | ARL8A | 3.15E−27 | 0.285 | 0.115 | 0.124 | 3.633E−23 |
| | | | | | | TMX4 | 3.42E−27 | 0.259 | 0.122 | 0.141 | 3.941E−23 |
| | | | | | | HPRT1 | 3.99E−27 | 0.332 | 0.136 | 0.132 | 4.604E−23 |
| | | | | | | B4GALT1 | 4.92E−27 | 0.250 | 0.135 | 0.189 | 5.684E−23 |
| | | | | | | TMEM106C | 5.54E−27 | 0.415 | 0.218 | 0.189 | 6.397E−23 |
| | | | | | | FANCI | 6.27E−27 | 0.403 | 0.172 | 0.118 | 7.237E−23 |
| | | | | | | POLR3K | 1.06E−26 | 0.253 | 0.128 | 0.146 | 1.226E−22 |
| | | | | | | POLD2 | 1.13E−26 | 0.309 | 0.133 | 0.118 | 1.310E−22 |
| | | | | | | DUSP23 | 1.24E−26 | 0.269 | 0.129 | 0.138 | 1.435E−22 |
| | | | | | | PTPRK | 1.32E−26 | 0.378 | 0.186 | 0.183 | 1.528E−22 |
| | | | | | | MRPL42 | 1.41E−26 | 0.427 | 0.142 | 0.101 | 1.629E−22 |
| | | | | | | CKLF | 2.24E−26 | 0.265 | 0.131 | 0.138 | 2.589E−22 |
| | | | | | | SMG7 | 2.30E−26 | 0.311 | 0.146 | 0.161 | 2.659E−22 |
| | | | | | | TBC1D23 | 2.53E−26 | 0.334 | 0.137 | 0.11 | 2.921E−22 |
| | | | | | | TMX3 | 2.98E−26 | 0.303 | 0.101 | 0.104 | 3.434E−22 |
| | | | | | | MRPL32 | 3.29E−26 | 0.391 | 0.173 | 0.115 | 3.794E−22 |
| | | | | | | RPF2 | 3.38E−26 | 0.426 | 0.208 | 0.163 | 3.905E−22 |
| | | | | | | APIP | 3.41E−26 | 0.294 | 0.129 | 0.13 | 3.930E−22 |
| | | | | | | RPP38 | 4.23E−26 | 0.343 | 0.132 | 0.096 | 4.880E−22 |
| | | | | | | RSU1 | 4.64E−26 | 0.563 | 0.182 | 0.017 | 5.358E−22 |
| | | | | | | SMC5 | 4.93E−26 | 0.462 | 0.159 | 0.099 | 5.689E−22 |
| | | | | | | CCNC | 5.40E−26 | 0.343 | 0.148 | 0.124 | 6.235E−22 |
| | | | | | | CDC42BPA | 6.11E−26 | 0.396 | 0.201 | 0.192 | 7.056E−22 |
| | | | | | | UBXN2A | 8.98E−26 | 0.302 | 0.128 | 0.166 | 1.036E−21 |
| | | | | | | MLLT4 | 1.06E−25 | 0.282 | 0.137 | 0.18 | 1.224E−21 |
| | | | | | | TMCO3 | 1.29E−25 | 0.419 | 0.187 | 0.121 | 1.486E−21 |
| | | | | | | PRPF38A | 1.31E−25 | 0.337 | 0.13 | 0.118 | 1.510E−21 |
| | | | | | | SMARCA1 | 1.46E−25 | 0.388 | 0.172 | 0.177 | 1.685E−21 |
| | | | | | | CRYAB | 1.54E−25 | 0.424 | 0.215 | 0.135 | 1.779E−21 |
| | | | | | | PNISR | 1.55E−25 | 0.296 | 0.173 | 0.217 | 1.791E−21 |
| | | | | | | ERP27 | 1.72E−25 | 0.484 | 0.207 | 0.138 | 1.988E−21 |
| | | | | | | ANXA3 | 1.83E−25 | 0.567 | 0.177 | 0.008 | 2.111E−21 |
| | | | | | | TMEM63A | 2.49E−25 | 0.419 | 0.157 | 0.042 | 2.869E−21 |
| | | | | | | CTSB | 2.63E−25 | 0.367 | 0.169 | 0.127 | 3.032E−21 |
| | | | | | | ITSN2 | 2.89E−25 | 0.256 | 0.147 | 0.223 | 3.337E−21 |
| | | | | | | TTC17 | 3.00E−25 | 0.367 | 0.208 | 0.211 | 3.463E−21 |
| | | | | | | NSL1 | 3.51E−25 | 0.418 | 0.147 | 0.093 | 4.055E−21 |
| | | | | | | CXADR | 3.63E−25 | 0.497 | 0.195 | 0.104 | 4.184E−21 |
| | | | | | | LAD1 | 4.40E−25 | 0.494 | 0.188 | 0.073 | 5.075E−21 |
| | | | | | | USP16 | 5.35E−25 | 0.311 | 0.179 | 0.217 | 6.180E−21 |
| | | | | | | TF | 5.99E−25 | 0.791 | 0.271 | 0.115 | 6.912E−21 |
| | | | | | | HLA-DRA | 6.67E−25 | 0.321 | 0.166 | 0.113 | 7.694E−21 |
| | | | | | | TANK | 7.52E−25 | 0.439 | 0.197 | 0.197 | 8.681E−21 |
| | | | | | | NR2F2 | 7.56E−25 | 0.259 | 0.119 | 0.115 | 8.730E−21 |
| | | | | | | CHD2 | 8.32E−25 | 0.266 | 0.175 | 0.208 | 9.601E−21 |
| | | | | | | XRN1 | 8.37E−25 | 0.314 | 0.163 | 0.169 | 9.665E−21 |
| | | | | | | PCID2 | 8.69E−25 | 0.401 | 0.181 | 0.127 | 1.003E−20 |
| | | | | | | PSME2 | 9.16E−25 | 0.327 | 0.174 | 0.186 | 1.058E−20 |
| | | | | | | CDKN2A | 9.32E−25 | 0.499 | 0.186 | 0.045 | 1.076E−20 |
| | | | | | | SKA2 | 9.72E−25 | 0.418 | 0.172 | 0.118 | 1.122E−20 |
| | | | | | | DIAPH2 | 1.31E−24 | 0.613 | 0.19 | 0.008 | 1.509E−20 |
| | | | | | | MACC1 | 1.52E−24 | 0.690 | 0.246 | 0.045 | 1.758E−20 |
| | | | | | | EBP | 1.74E−24 | 0.431 | 0.165 | 0.082 | 2.008E−20 |
| | | | | | | PDZK1IP1 | 1.90E−24 | 0.470 | 0.168 | 0.014 | 2.194E−20 |
| | | | | | | FNDC3B | 2.05E−24 | 0.454 | 0.179 | 0.11 | 2.361E−20 |
| | | | | | | C16orf91 | 2.19E−24 | 0.284 | 0.134 | 0.127 | 2.526E−20 |
| | | | | | | MRAS | 2.31E−24 | 0.484 | 0.163 | 0.006 | 2.666E−20 |
| | | | | | | EDARADD | 2.48E−24 | 0.299 | 0.192 | 0.172 | 2.864E−20 |
| | | | | | | HNRNPH2 | 2.65E−24 | 0.259 | 0.124 | 0.146 | 3.058E−20 |
| | | | | | | MED4 | 4.36E−24 | 0.358 | 0.198 | 0.194 | 5.031E−20 |
| | | | | | | NMI | 4.38E−24 | 0.396 | 0.159 | 0.155 | 5.053E−20 |
| | | | | | | WWTR1 | 4.44E−24 | 0.467 | 0.163 | 0.056 | 5.123E−20 |
| | | | | | | KIF21A | 5.31E−24 | 0.318 | 0.126 | 0.124 | 6.123E−20 |
| | | | | | | PNPT1 | 6.25E−24 | 0.312 | 0.162 | 0.172 | 7.208E−20 |
| | | | | | | TRIM2 | 6.40E−24 | 0.442 | 0.198 | 0.149 | 7.388E−20 |
| | | | | | | RFK | 9.08E−24 | 0.429 | 0.139 | 0.051 | 1.048E−19 |
| | | | | | | FAM20B | 9.13E−24 | 0.258 | 0.111 | 0.11 | 1.054E−19 |
| | | | | | | TCN1 | 9.46E−24 | 0.773 | 0.17 | 0 | 1.092E−19 |
| | | | | | | HORMAD1 | 1.26E−23 | 0.527 | 0.193 | 0.02 | 1.454E−19 |
| | | | | | | OPN3 | 1.38E−23 | 0.365 | 0.131 | 0.068 | 1.588E−19 |
| | | | | | | PIK3R1 | 1.39E−23 | 0.407 | 0.194 | 0.18 | 1.604E−19 |
| | | | | | | PMP22 | 1.48E−23 | 0.338 | 0.205 | 0.2 | 1.714E−19 |
| | | | | | | PDHX | 1.61E−23 | 0.369 | 0.169 | 0.115 | 1.861E−19 |
| | | | | | | C2orf15 | 1.94E−23 | 0.334 | 0.142 | 0.146 | 2.243E−19 |
| | | | | | | BID | 2.85E−23 | 0.311 | 0.13 | 0.099 | 3.292E−19 |
| | | | | | | HDDC2 | 3.07E−23 | 0.270 | 0.145 | 0.163 | 3.545E−19 |
| | | | | | | MKI67 | 3.25E−23 | 0.770 | 0.316 | 0.149 | 3.745E−19 |
| | | | | | | PRKX | 4.81E−23 | 0.576 | 0.185 | 0.006 | 5.548E−19 |
| | | | | | | TMEM176A | 5.01E−23 | 0.523 | 0.179 | 0.025 | 5.779E−19 |
| | | | | | | IL32 | 6.22E−23 | 0.545 | 0.181 | 0.003 | 7.173E−19 |
| | | | | | | TMEM97 | 6.24E−23 | 0.256 | 0.095 | 0.118 | 7.199E−19 |
| | | | | | | HSPA14 | 6.78E−23 | 0.412 | 0.181 | 0.146 | 7.822E−19 |
| | | | | | | RNF168 | 7.64E−23 | 0.295 | 0.175 | 0.211 | 8.816E−19 |
| | | | | | | SLFN5 | 1.06E−22 | 0.539 | 0.171 | 0.031 | 1.220E−18 |
| | | | | | | NCK1 | 1.41E−22 | 0.362 | 0.133 | 0.082 | 1.628E−18 |
| | | | | | | PHF11 | 1.44E−22 | 0.344 | 0.141 | 0.115 | 1.659E−18 |
| | | | | | | NOL11 | 1.52E−22 | 0.301 | 0.105 | 0.085 | 1.759E−18 |
| | | | | | | TRIO | 1.55E−22 | 0.284 | 0.132 | 0.124 | 1.789E−18 |
| | | | | | | TPP2 | 1.60E−22 | 0.324 | 0.117 | 0.093 | 1.850E−18 |
| | | | | | | SPEN | 1.78E−22 | 0.299 | 0.155 | 0.177 | 2.050E−18 |
| | | | | | | COMMD2 | 1.88E−22 | 0.578 | 0.228 | 0.09 | 2.167E−18 |
| | | | | | | RTP4 | 1.95E−22 | 0.507 | 0.171 | 0.003 | 2.249E−18 |
| | | | | | | CBWD1 | 2.00E−22 | 0.405 | 0.149 | 0.082 | 2.312E−18 |
| | | | | | | DDX58 | 2.02E−22 | 0.464 | 0.158 | 0.062 | 2.332E−18 |
| | | | | | | PARP14 | 3.17E−22 | 0.480 | 0.199 | 0.124 | 3.660E−18 |
| | | | | | | PKIB | 3.21E−22 | 0.334 | 0.142 | 0.135 | 3.704E−18 |
| | | | | | | TRIM44 | 3.23E−22 | 0.360 | 0.165 | 0.115 | 3.722E−18 |
| | | | | | | METTL7A | 3.81E−22 | 0.301 | 0.111 | 0.087 | 4.401E−18 |
| | | | | | | TOPBP1 | 4.30E−22 | 0.372 | 0.165 | 0.11 | 4.960E−18 |
| | | | | | | HLA-F | 4.57E−22 | 0.356 | 0.137 | 0.065 | 5.269E−18 |
| | | | | | | DENND1B | 4.63E−22 | 0.394 | 0.179 | 0.135 | 5.348E−18 |
| | | | | | | ECT2 | 5.03E−22 | 0.436 | 0.177 | 0.141 | 5.810E−18 |
| | | | | | | MFSD1 | 5.16E−22 | 0.374 | 0.144 | 0.073 | 5.950E−18 |
| | | | | | | PUS7L | 5.77E−22 | 0.494 | 0.177 | 0.076 | 6.664E−18 |
| | | | | | | KDM3A | 6.04E−22 | 0.257 | 0.135 | 0.155 | 6.968E−18 |
| | | | | | | EGLN1 | 8.04E−22 | 0.259 | 0.137 | 0.186 | 9.279E−18 |
| | | | | | | ZNF292 | 8.06E−22 | 0.376 | 0.186 | 0.161 | 9.299E−18 |
| | | | | | | NVL | 8.78E−22 | 0.260 | 0.102 | 0.104 | 1.013E−17 |
| | | | | | | C1orf27 | 9.02E−22 | 0.361 | 0.167 | 0.13 | 1.042E−17 |
| | | | | | | FH | 9.54E−22 | 0.315 | 0.158 | 0.141 | 1.101E−17 |
| | | | | | | DDX60 | 9.86E−22 | 0.478 | 0.172 | 0.051 | 1.138E−17 |
| | | | | | | NGRN | 1.07E−21 | 0.263 | 0.102 | 0.085 | 1.231E−17 |
| | | | | | | CCL5 | 1.08E−21 | 0.548 | 0.156 | 0.011 | 1.249E−17 |
| | | | | | | ACTN1 | 1.09E−21 | 0.284 | 0.13 | 0.11 | 1.254E−17 |
| | | | | | | SESTD1 | 1.09E−21 | 0.328 | 0.113 | 0.07 | 1.257E−17 |
| | | | | | | CD14 | 1.12E−21 | 0.351 | 0.124 | 0.059 | 1.291E−17 |
| | | | | | | KCNK1 | 1.13E−21 | 0.287 | 0.109 | 0.079 | 1.300E−17 |
| | | | | | | HAPLN3 | 1.23E−21 | 0.459 | 0.156 | 0 | 1.416E−17 |
| | | | | | | ZBTB41 | 1.23E−21 | 0.329 | 0.146 | 0.113 | 1.418E−17 |
| | | | | | | PPIP5K2 | 1.44E−21 | 0.256 | 0.117 | 0.141 | 1.662E−17 |
| | | | | | | TYMP | 1.50E−21 | 0.264 | 0.13 | 0.124 | 1.736E−17 |
| | | | | | | DNAH14 | 1.64E−21 | 0.336 | 0.123 | 0.082 | 1.889E−17 |
| | | | | | | CKAP2 | 1.89E−21 | 0.387 | 0.12 | 0.068 | 2.181E−17 |
| | | | | | | OXR1 | 2.00E−21 | 0.403 | 0.167 | 0.135 | 2.304E−17 |
| | | | | | | CEP70 | 2.13E−21 | 0.528 | 0.199 | 0.146 | 2.460E−17 |
| | | | | | | TCF12 | 2.14E−21 | 0.252 | 0.118 | 0.144 | 2.465E−17 |
| | | | | | | ATG5 | 2.23E−21 | 0.346 | 0.149 | 0.085 | 2.575E−17 |
| | | | | | | KPNA2 | 2.25E−21 | 0.261 | 0.13 | 0.155 | 2.601E−17 |
| | | | | | | TIFA | 2.61E−21 | 0.383 | 0.132 | 0.045 | 3.018E−17 |
| | | | | | | COMMD9 | 2.65E−21 | 0.363 | 0.133 | 0.056 | 3.053E−17 |
| | | | | | | CYP51A1 | 2.89E−21 | 0.393 | 0.153 | 0.085 | 3.338E−17 |
| | | | | | | CHSY1 | 3.70E−21 | 0.268 | 0.101 | 0.059 | 4.274E−17 |
| | | | | | | APOL6 | 3.77E−21 | 0.547 | 0.196 | 0.076 | 4.356E−17 |
| | | | | | | BLZF1 | 4.19E−21 | 0.407 | 0.141 | 0.045 | 4.836E−17 |
| | | | | | | PGBD5 | 5.08E−21 | 0.443 | 0.162 | 0.011 | 5.864E−17 |
| | | | | | | AHCTF1 | 5.23E−21 | 0.341 | 0.13 | 0.096 | 6.038E−17 |
| | | | | | | IFI44 | 5.33E−21 | 0.451 | 0.163 | 0.039 | 6.154E−17 |
| | | | | | | SCD5 | 6.23E−21 | 0.493 | 0.162 | 0.048 | 7.192E−17 |
| | | | | | | RABGAP1L | 6.39E−21 | 0.350 | 0.136 | 0.07 | 7.370E−17 |
| | | | | | | HSPA13 | 6.74E−21 | 0.287 | 0.103 | 0.048 | 7.774E−17 |
| | | | | | | GLRX2 | 8.55E−21 | 0.410 | 0.167 | 0.062 | 9.868E−17 |
| | | | | | | AIM1 | 9.70E−21 | 0.311 | 0.122 | 0.093 | 1.120E−16 |
| | | | | | | INSR | 9.93E−21 | 0.446 | 0.176 | 0.087 | 1.145E−16 |
| | | | | | | ITGA6 | 1.09E−20 | 0.300 | 0.129 | 0.104 | 1.256E−16 |
| | | | | | | HEATR1 | 1.34E−20 | 0.302 | 0.104 | 0.096 | 1.546E−16 |
| | | | | | | CDKAL1 | 1.40E−20 | 0.454 | 0.172 | 0.068 | 1.619E−16 |
| | | | | | | CINP | 1.55E−20 | 0.289 | 0.115 | 0.124 | 1.786E−16 |
| | | | | | | ACBD5 | 1.56E−20 | 0.488 | 0.165 | 0.073 | 1.800E−16 |
| | | | | | | CUL2 | 1.88E−20 | 0.295 | 0.121 | 0.104 | 2.166E−16 |
| | | | | | | SVIL | 1.99E−20 | 0.354 | 0.15 | 0.09 | 2.301E−16 |
| | | | | | | DTX3L | 2.26E−20 | 0.357 | 0.134 | 0.087 | 2.603E−16 |
| | | | | | | FAIM | 2.65E−20 | 0.291 | 0.109 | 0.07 | 3.058E−16 |
| | | | | | | CALD1 | 2.98E−20 | 0.398 | 0.15 | 0.101 | 3.443E−16 |
| | | | | | | ACOT9 | 3.10E−20 | 0.472 | 0.172 | 0.059 | 3.580E−16 |
| | | | | | | CTSC | 3.13E−20 | 0.309 | 0.121 | 0.093 | 3.613E−16 |
| | | | | | | EIF2B3 | 4.01E−20 | 0.303 | 0.129 | 0.101 | 4.624E−16 |
| | | | | | | TBC1D15 | 4.11E−20 | 0.321 | 0.117 | 0.138 | 4.748E−16 |
| | | | | | | GLRX | 4.48E−20 | 0.405 | 0.137 | 0.042 | 5.173E−16 |
| | | | | | | STAG1 | 5.34E−20 | 0.351 | 0.169 | 0.127 | 6.159E−16 |
| | | | | | | CAMSAP2 | 5.52E−20 | 0.461 | 0.174 | 0.079 | 6.368E−16 |
| | | | | | | ARHGAP18 | 6.69E−20 | 0.310 | 0.126 | 0.093 | 7.724E−16 |
| | | | | | | KIAA1217 | 6.80E−20 | 0.270 | 0.106 | 0.107 | 7.842E−16 |
| | | | | | | COMMD8 | 7.78E−20 | 0.401 | 0.129 | 0.054 | 8.973E−16 |
| | | | | | | XAF1 | 8.16E−20 | 0.546 | 0.184 | 0.039 | 9.420E−16 |
| | | | | | | PTPN14 | 8.19E−20 | 0.387 | 0.133 | 0.034 | 9.455E−16 |
| | | | | | | IFIT3 | 1.01E−19 | 0.604 | 0.199 | 0.034 | 1.166E−15 |
| | | | | | | C1orf198 | 1.45E−19 | 0.307 | 0.131 | 0.087 | 1.673E−15 |
| | | | | | | GCNT2 | 1.64E−19 | 0.402 | 0.126 | 0.028 | 1.890E−15 |
| | | | | | | OAS2 | 1.86E−19 | 0.291 | 0.108 | 0.059 | 2.147E−15 |
| | | | | | | BPNT1 | 1.87E−19 | 0.458 | 0.147 | 0.056 | 2.153E−15 |
| | | | | | | IFIT1 | 2.63E−19 | 0.537 | 0.202 | 0.056 | 3.035E−15 |
| | | | | | | GTF3C6 | 2.74E−19 | 0.369 | 0.162 | 0.076 | 3.157E−15 |
| | | | | | | PTRH2 | 3.47E−19 | 0.270 | 0.113 | 0.115 | 4.007E−15 |
| | | | | | | THAP9-AS1 | 3.59E−19 | 0.287 | 0.101 | 0.079 | 4.141E−15 |
| | | | | | | SLC44A2 | 4.74E−19 | 0.282 | 0.129 | 0.107 | 5.474E−15 |
| | | | | | | ZCCHC6 | 5.00E−19 | 0.293 | 0.119 | 0.113 | 5.772E−15 |
| | | | | | | DTD1 | 5.13E−19 | 0.435 | 0.153 | 0.115 | 5.915E−15 |
| | | | | | | FAM46A | 5.62E−19 | 0.463 | 0.158 | 0.062 | 6.486E−15 |
| | | | | | | S100P | 6.20E−19 | 0.533 | 0.186 | 0.051 | 7.160E−15 |
| | | | | | | RAB3GAP2 | 8.34E−19 | 0.316 | 0.141 | 0.135 | 9.622E−15 |
| | | | | | | ITGB8 | 8.48E−19 | 0.449 | 0.144 | 0.02 | 9.792E−15 |
| | | | | | | ATR | 8.54E−19 | 0.328 | 0.111 | 0.073 | 9.857E−15 |
| | | | | | | PRPS2 | 1.05E−18 | 0.447 | 0.163 | 0.051 | 1.210E−14 |
| | | | | | | AARD | 1.13E−18 | 0.491 | 0.189 | 0.09 | 1.300E−14 |
| | | | | | | RAP2A | 1.39E−18 | 0.462 | 0.152 | 0.011 | 1.608E−14 |
| | | | | | | RASAL2 | 1.70E−18 | 0.366 | 0.124 | 0.011 | 1.961E−14 |
| | | | | | | TSIX | 1.71E−18 | 0.430 | 0.145 | 0.006 | 1.978E−14 |
| | | | | | | CDK6 | 1.77E−18 | 0.491 | 0.173 | 0.039 | 2.048E−14 |
| | | | | | | FAM129A | 1.88E−18 | 0.305 | 0.142 | 0.132 | 2.171E−14 |
| | | | | | | VLDLR | 1.90E−18 | 0.280 | 0.101 | 0.048 | 2.192E−14 |
| | | | | | | TRIP13 | 2.11E−18 | 0.335 | 0.12 | 0.054 | 2.436E−14 |
| | | | | | | PARP4 | 2.40E−18 | 0.359 | 0.111 | 0.042 | 2.774E−14 |
| | | | | | | SCYL2 | 2.90E−18 | 0.318 | 0.121 | 0.099 | 3.350E−14 |
| | | | | | | ANKRD36C | 3.16E−18 | 0.519 | 0.198 | 0.09 | 3.645E−14 |
| | | | | | | FAM111B | 4.26E−18 | 0.530 | 0.194 | 0.141 | 4.913E−14 |
| | | | | | | LYRM2 | 5.15E−18 | 0.267 | 0.115 | 0.107 | 5.938E−14 |
| | | | | | | NUSAP1 | 5.21E−18 | 0.354 | 0.133 | 0.093 | 6.017E−14 |
| | | | | | | CD74 | 5.36E−18 | 0.384 | 0.186 | 0.093 | 6.190E−14 |
| | | | | | | SUZ12 | 5.79E−18 | 0.370 | 0.169 | 0.087 | 6.677E−14 |
| | | | | | | CCDC59 | 6.56E−18 | 0.342 | 0.127 | 0.079 | 7.567E−14 |
| | | | | | | TWISTNB | 7.14E−18 | 0.403 | 0.152 | 0.104 | 8.246E−14 |
| | | | | | | THBS1 | 7.42E−18 | 0.337 | 0.107 | 0.006 | 8.559E−14 |
| | | | | | | SUCLA2 | 7.63E−18 | 0.279 | 0.102 | 0.056 | 8.804E−14 |
| | | | | | | EZH2 | 7.68E−18 | 0.316 | 0.134 | 0.127 | 8.863E−14 |
| | | | | | | CASP8AP2 | 8.49E−18 | 0.328 | 0.123 | 0.099 | 9.797E−14 |
| | | | | | | RCAN1 | 9.03E−18 | 0.356 | 0.141 | 0.104 | 1.043E−13 |
| | | | | | | TOR1AIP1 | 1.33E−17 | 0.397 | 0.149 | 0.068 | 1.531E−13 |
| | | | | | | CD109 | 2.07E−17 | 0.401 | 0.137 | 0.034 | 2.386E−13 |
| | | | | | | KLF5 | 2.14E−17 | 0.296 | 0.103 | 0.031 | 2.473E−13 |
| | | | | | | ASPM | 2.16E−17 | 0.579 | 0.184 | 0.085 | 2.496E−13 |
| | | | | | | SULT1B1 | 2.37E−17 | 0.454 | 0.142 | 0.003 | 2.739E−13 |
| | | | | | | SOAT1 | 2.79E−17 | 0.340 | 0.118 | 0.068 | 3.222E−13 |
| | | | | | | ANO6 | 2.90E−17 | 0.355 | 0.134 | 0.073 | 3.344E−13 |
| | | | | | | IRF6 | 3.18E−17 | 0.288 | 0.135 | 0.099 | 3.675E−13 |
| | | | | | | IFIT2 | 3.27E−17 | 0.389 | 0.139 | 0.042 | 3.777E−13 |
| | | | | | | TMEM176B | 4.24E−17 | 0.440 | 0.149 | 0.025 | 4.889E−13 |
| | | | | | | QSOX1 | 4.91E−17 | 0.465 | 0.188 | 0.076 | 5.666E−13 |
| | | | | | | HPS3 | 5.06E−17 | 0.358 | 0.139 | 0.093 | 5.839E−13 |
| | | | | | | TAP1 | 5.11E−17 | 0.269 | 0.141 | 0.115 | 5.900E−13 |
| | | | | | | ICK | 5.11E−17 | 0.302 | 0.115 | 0.079 | 5.902E−13 |
| | | | | | | CYBA | 5.29E−17 | 0.419 | 0.163 | 0.054 | 6.109E−13 |
| | | | | | | LIN9 | 6.62E−17 | 0.341 | 0.104 | 0.042 | 7.643E−13 |
| | | | | | | AKT3 | 7.02E−17 | 0.419 | 0.123 | 0 | 8.099E−13 |
| | | | | | | C5orf46 | 7.14E−17 | 0.420 | 0.139 | 0.003 | 8.240E−13 |
| | | | | | | GPR180 | 7.34E−17 | 0.331 | 0.116 | 0.045 | 8.475E−13 |
| | | | | | | TRIM14 | 8.92E−17 | 0.415 | 0.141 | 0.017 | 1.030E−12 |
| | | | | | | LASP1 | 1.23E−16 | 0.324 | 0.132 | 0.054 | 1.417E−12 |
| | | | | | | NDRG2 | 1.74E−16 | 0.296 | 0.158 | 0.107 | 2.010E−12 |
| | | | | | | ATP5E | 1.81E−16 | 0.334 | 0.788 | 0.837 | 2.091E−12 |
| | | | | | | CXCL17 | 2.10E−16 | 0.365 | 0.119 | 0.006 | 2.426E−12 |
| | | | | | | GBP1 | 2.11E−16 | 0.406 | 0.128 | 0.011 | 2.438E−12 |
| | | | | | | IFI44L | 2.21E−16 | 0.419 | 0.157 | 0.056 | 2.554E−12 |
| | | | | | | HIST1H2BC | 2.79E−16 | 0.261 | 0.103 | 0.096 | 3.223E−12 |
| | | | | | | MAP7D1 | 3.97E−16 | 0.274 | 0.102 | 0.076 | 4.579E−12 |
| | | | | | | MIR4435- | 4.78E−16 | 0.406 | 0.128 | 0.008 | 5.514E−12 |
| | | | | | | 1HG | | | | | |
| | | | | | | PSMB9 | 5.94E−16 | 0.320 | 0.116 | 0.051 | 6.853E−12 |
| | | | | | | PIK3CA | 9.88E−16 | 0.306 | 0.118 | 0.09 | 1.141E−11 |
| | | | | | | RAB7L1 | 1.09E−15 | 0.284 | 0.11 | 0.073 | 1.255E−11 |
| | | | | | | YAF2 | 1.10E−15 | 0.276 | 0.109 | 0.096 | 1.268E−11 |
| | | | | | | CBR3 | 1.26E−15 | 0.266 | 0.103 | 0.076 | 1.458E−11 |
| | | | | | | PMAIP1 | 1.31E−15 | 0.410 | 0.141 | 0.028 | 1.514E−11 |
| | | | | | | GNAQ | 1.88E−15 | 0.256 | 0.113 | 0.11 | 2.169E−11 |
| | | | | | | MLK4 | 2.13E−15 | 0.400 | 0.12 | 0.003 | 2.460E−11 |
| | | | | | | FSTL1 | 2.66E−15 | 0.373 | 0.124 | 0.011 | 3.069E−11 |
| | | | | | | SYTL4 | 3.69E−15 | 0.339 | 0.109 | 0.003 | 4.261E−11 |
| | | | | | | MYNN | 7.06E−15 | 0.263 | 0.119 | 0.101 | 8.144E−11 |
| | | | | | | NLRP2 | 8.32E−15 | 0.365 | 0.118 | 0.025 | 9.604E−11 |
| | | | | | | ATL2 | 9.22E−15 | 0.315 | 0.105 | 0.045 | 1.064E−10 |
| | | | | | | MTRNR2L11 | 1.18E−14 | 0.360 | 0.172 | 0.09 | 1.362E−10 |
| | | | | | | TAOK1 | 1.28E−14 | 0.267 | 0.105 | 0.217 | 1.482E−10 |
| | | | | | | BACE2 | 1.45E−14 | 0.326 | 0.135 | 0.065 | 1.668E−10 |
| | | | | | | DAPP1 | 1.46E−14 | 0.419 | 0.133 | 0.011 | 1.681E−10 |
| | | | | | | DIEXF | 2.62E−14 | 0.299 | 0.108 | 0.039 | 3.021E−10 |
| | | | | | | APOBEC3B | 3.07E−14 | 0.337 | 0.114 | 0.011 | 3.544E−10 |
| | | | | | | PROM1 | 4.20E−14 | 0.311 | 0.104 | 0 | 4.844E−10 |
| | | | | | | MAP1B | 4.24E−14 | 0.332 | 0.172 | 0.09 | 4.893E−10 |
| | | | | | | TNFSF13B | 4.38E−14 | 0.447 | 0.126 | 0.017 | 5.053E−10 |
| | | | | | | EDN1 | 4.99E−14 | 0.314 | 0.16 | 0.124 | 5.755E−10 |
| | | | | | | ZDHHC20 | 5.96E−14 | 0.324 | 0.121 | 0.039 | 6.884E−10 |
| | | | | | | B3GNT5 | 6.26E−14 | 0.372 | 0.113 | 0.003 | 7.225E−10 |
| | | | | | | LPL | 6.28E−14 | 0.309 | 0.101 | 0.011 | 7.247E−10 |
| | | | | | | AP1AR | 6.64E−14 | 0.262 | 0.102 | 0.07 | 7.664E−10 |
| | | | | | | MYO5B | 6.78E−14 | 0.278 | 0.108 | 0.062 | 7.820E−10 |
| | | | | | | SMYD2 | 7.59E−14 | 0.324 | 0.101 | 0.034 | 8.763E−10 |
| | | | | | | MT-RNR1 | 1.04E−13 | 0.703 | 0.973 | 0.986 | 1.199E−09 |
| | | | | | | NDUFAF4 | 1.27E−13 | 0.318 | 0.128 | 0.062 | 1.462E−09 |
| | | | | | | DCUN1D4 | 1.38E−13 | 0.325 | 0.129 | 0.093 | 1.598E−09 |
| | | | | | | MTRNR2L1 | 1.45E−13 | 0.267 | 0.877 | 0.932 | 1.669E−09 |
| | | | | | | HSP90AA1 | 1.51E−13 | 0.297 | 0.916 | 0.93 | 1.745E−09 |
| | | | | | | SAMD9L | 1.99E−13 | 0.447 | 0.121 | 0.014 | 2.293E−09 |
| | | | | | | WAC-AS1 | 2.58E−13 | 0.310 | 0.128 | 0.101 | 2.972E−09 |
| | | | | | | PTTG1 | 2.69E−13 | 0.337 | 0.17 | 0.127 | 3.107E−09 |
| | | | | | | BRD9 | 2.81E−13 | 0.299 | 0.136 | 0.093 | 3.239E−09 |
| | | | | | | IFIT5 | 3.81E−13 | 0.291 | 0.126 | 0.059 | 4.399E−09 |
| | | | | | | UGT8 | 5.50E−13 | 0.389 | 0.136 | 0.051 | 6.348E−09 |
| | | | | | | MSN | 6.49E−13 | 0.405 | 0.15 | 0.028 | 7.488E−09 |
| | | | | | | OSBPL8 | 1.07E−12 | 0.313 | 0.12 | 0.082 | 1.233E−08 |
| | | | | | | PLS3 | 1.84E−12 | 0.315 | 0.118 | 0.028 | 2.124E−08 |
| | | | | | | OAS3 | 2.40E−12 | 0.282 | 0.102 | 0.034 | 2.765E−08 |
| | | | | | | STIM1 | 2.75E−12 | 0.263 | 0.114 | 0.048 | 3.169E−08 |
| | | | | | | DIAPH3 | 3.06E−12 | 0.314 | 0.102 | 0.039 | 3.527E−08 |
| | | | | | | SAMD9 | 4.19E−12 | 0.382 | 0.117 | 0.017 | 4.837E−08 |
| | | | | | | EPSTI1 | 6.70E−12 | 0.343 | 0.117 | 0.028 | 7.731E−08 |
| | | | | | | HIST1H1D | 2.80E−11 | 0.320 | 0.165 | 0.146 | 3.231E−07 |
| | | | | | | ST3GAL6 | 3.49E−11 | 0.330 | 0.103 | 0.008 | 4.032E−07 |
| | | | | | | SLC25A37 | 5.11E−11 | 0.251 | 0.101 | 0.059 | 5.900E−07 |
| | | | | | | NES | 9.71E−11 | 0.328 | 0.113 | 0.02 | 1.120E−06 |
| | | | | | | STEAP4 | 6.13E−10 | 0.335 | 0.113 | 0.023 | 7.071E−06 |
| | | | | | | TLE4 | 5.87E−09 | 0.320 | 0.107 | 0.039 | 6.771E−05 |
| | | | | | | SECTM1 | 1.20E−08 | 0.309 | 0.114 | 0.025 | 1.382E−04 |

| CSF.patient.043.TvUT.tumor.bimod.markers.dn.l | | | | | | CSF.patient.043.TvUT.tumor.bimod.markers.up.1 | | | | | |
|---|---|---|---|---|---|---|---|---|---|---|---|
| | p_val | avg_logFC | pct.1 | pct.2 | p_val_adj | | p_val | avg_logFC | pct.1 | pct.2 | p_val_adj |
| RPL23A | 7.47E−46 | −1.544 | 0.5 | 0.843 | 7.373E−42 | PDHX | 7.59E−31 | 1.931 | 0.87 | 0.488 | 7.491E−27 |
| RPS25 | 2.66E−43 | −1.596 | 0.472 | 0.815 | 2.627E−39 | HIST1H4C | 1.48E−28 | 2.155 | 0.713 | 0.149 | 1.461E−24 |
| RPS18 | 1.27E−42 | −1.484 | 0.833 | 0.968 | 1.256E−38 | RAD21 | 3.95E−25 | 2.163 | 0.648 | 0.278 | 3.898E−21 |
| MALAT1 | 9.48E−39 | −1.692 | 0.981 | 1 | 9.355E−35 | SPCS3 | 6.21E−25 | 0.743 | 0.648 | 0.101 | 6.130E−21 |
| MT-RNR2 | 5.08E−37 | −1.235 | 1 | 1 | 5.012E−33 | PSMB6 | 3.00E−24 | 0.396 | 0.704 | 0.177 | 2.964E−20 |
| MTRNR2L1 | 1.33E−35 | −1.887 | 0.648 | 0.919 | 1.317E−31 | GADD45GIP1 | 3.73E−24 | 0.325 | 0.676 | 0.169 | 3.679E−20 |
| RPS7 | 3.83E−31 | −1.265 | 0.361 | 0.625 | 3.785E−27 | SNRPB2 | 5.07E−24 | 0.296 | 0.704 | 0.202 | 5.007E−20 |
| RPL12 | 4.54E−29 | −0.785 | 0.954 | 1 | 4.478E−25 | SSR3 | 2.42E−23 | 0.751 | 0.769 | 0.218 | 2.388E−19 |
| RNA28S5 | 6.94E−29 | −1.616 | 0.63 | 0.73 | 6.848E−25 | UTP11L | 2.71E−23 | 0.304 | 0.593 | 0.117 | 2.674E−19 |
| MT-CO1 | 1.03E−27 | −1.566 | 0.62 | 0.843 | 1.018E−23 | PSME1 | 5.55E−23 | 0.720 | 0.796 | 0.222 | 5.478E−19 |
| RPS27 | 5.12E−27 | −1.240 | 0.315 | 0.544 | 5.052E−23 | OCIAD1 | 7.15E−23 | 0.357 | 0.685 | 0.181 | 7.053E−19 |
| MT-ND1 | 1.58E−26 | −1.255 | 0.426 | 0.585 | 1.561E−22 | PSMA5 | 1.03E−22 | 0.257 | 0.583 | 0.137 | 1.012E−18 |
| NBEAL1 | 8.78E−26 | −1.374 | 0.778 | 0.927 | 8.661E−22 | PPA1 | 1.35E−22 | 0.515 | 0.778 | 0.23 | 1.328E−18 |
| MT-CO2 | 3.64E−24 | −1.124 | 0.454 | 0.669 | 3.596E−20 | B2M | 5.02E−22 | 1.321 | 0.972 | 0.948 | 4.953E−18 |
| RPSA | 2.83E−23 | −0.562 | 0.38 | 0.363 | 2.789E−19 | COMT | 5.62E−22 | 0.274 | 0.556 | 0.113 | 5.544E−18 |
| MT-CO3 | 3.09E−22 | −0.954 | 0.787 | 0.931 | 3.054E−18 | FKBP3 | 6.48E−22 | 0.455 | 0.759 | 0.23 | 6.392E−18 |
| JUNB | 8.59E−22 | −0.726 | 0.444 | 0.419 | 8.474E−18 | CPNE3 | 7.70E−22 | 0.352 | 0.694 | 0.194 | 7.595E−18 |
| FTH1 | 2.21E−21 | −1.139 | 0.731 | 0.859 | 2.182E−17 | G3BP1 | 8.05E−22 | 0.286 | 0.62 | 0.157 | 7.949E−18 |
| MT-ATP6 | 3.45E−21 | −1.046 | 0.62 | 0.714 | 3.402E−17 | GLO1 | 9.24E−22 | 0.503 | 0.657 | 0.141 | 9.116E−18 |
| EEF1D | 5.09E−21 | −0.593 | 0.556 | 0.508 | 5.028E−17 | C19orf10 | 9.99E−22 | 0.645 | 0.611 | 0.105 | 9.856E−18 |
| RPL6 | 7.35E−21 | −0.498 | 0.685 | 0.516 | 7.252E−17 | TSPAN13 | 1.15E−21 | 0.292 | 0.537 | 0.101 | 1.139E−17 |
| TMA7 | 5.23E−20 | −0.321 | 0.509 | 0.302 | 5.160E−16 | RAD23A | 1.86E−21 | 0.417 | 0.685 | 0.173 | 1.835E−17 |
| SUMO2 | 6.24E−20 | −0.426 | 0.37 | 0.262 | 6.159E−16 | KDM1A | 3.12E−21 | 0.257 | 0.444 | 0.069 | 3.081E−17 |
| RPL29 | 7.98E−20 | −0.538 | 0.62 | 0.488 | 7.871E−16 | ATPIF1 | 5.26E−21 | 0.379 | 0.741 | 0.242 | 5.189E−17 |
| VMP1 | 1.32E−19 | −0.777 | 0.454 | 0.508 | 1.306E−15 | RPS16 | 7.29E−21 | 0.854 | 1 | 0.907 | 7.198E−17 |
| SFPQ | 3.29E−19 | −0.401 | 0.676 | 0.415 | 3.248E−15 | EMC4 | 9.69E−21 | 0.352 | 0.472 | 0.06 | 9.561E−17 |
| COX6C | 5.06E−19 | −0.735 | 0.713 | 0.633 | 4.996E−15 | 2-Sep | 9.83E−21 | 0.276 | 0.815 | 0.339 | 9.704E−17 |
| TMEM14C | 6.78E−19 | −0.263 | 0.454 | 0.23 | 6.692E−15 | HSP90B1 | 1.10E−20 | 1.300 | 0.907 | 0.52 | 1.083E−16 |
| YWHAZ | 3.22E−18 | −0.366 | 0.685 | 0.492 | 3.178E−14 | TAP1 | 1.13E−20 | 0.439 | 0.5 | 0.065 | 1.119E−16 |
| EEF1A1 | 4.33E−18 | −0.704 | 0.917 | 0.871 | 4.273E−14 | EIF2AK2 | 2.99E−20 | 0.365 | 0.648 | 0.169 | 2.952E−16 |
| NAMPT | 1.57E−17 | −0.592 | 0.324 | 0.31 | 1.552E−13 | LRPPRC | 3.24E−20 | 0.286 | 0.611 | 0.165 | 3.197E−16 |
| NEMF | 1.95E−17 | −0.332 | 0.472 | 0.266 | 1.924E−13 | HNRNPDL | 4.57E−20 | 0.256 | 0.75 | 0.282 | 4.506E−16 |
| BRWD1 | 2.29E−17 | −0.462 | 0.389 | 0.254 | 2.264E−13 | TTC37 | 5.48E−20 | 0.347 | 0.565 | 0.117 | 5.407E−16 |
| ANKRD30A | 5.07E−17 | −1.047 | 0.833 | 0.923 | 5.001E−13 | NDUFB6 | 8.40E−20 | 0.598 | 0.815 | 0.282 | 8.293E−16 |
| ATP5EP2 | 8.42E−17 | −0.417 | 0.481 | 0.367 | 8.312E−13 | TMCO1 | 1.79E−19 | 0.272 | 0.769 | 0.298 | 1.769E−15 |
| SRSF11 | 1.57E−16 | −0.329 | 0.62 | 0.371 | 1.550E−12 | COA6 | 2.25E−19 | 0.289 | 0.593 | 0.161 | 2.224E−15 |
| BZW1 | 1.65E−16 | −0.369 | 0.5 | 0.335 | 1.631E−12 | SMIM15 | 2.52E−19 | 0.290 | 0.435 | 0.065 | 2.486E−15 |
| UQCRB | 2.78E−16 | −0.604 | 0.63 | 0.548 | 2.746E−12 | ARF1 | 4.66E−19 | 0.456 | 0.759 | 0.262 | 4.595E−15 |
| DSP | 4.25E−16 | −0.544 | 0.546 | 0.448 | 4.194E−12 | METAP2 | 5.07E−19 | 0.303 | 0.602 | 0.181 | 5.006E−15 |
| TMSB4X | 4.38E−16 | −1.100 | 0.231 | 0.484 | 4.327E−12 | TKT | 7.39E−19 | 0.714 | 0.741 | 0.246 | 7.294E−15 |
| HMGN1 | 4.92E−16 | −0.253 | 0.574 | 0.363 | 4.859E−12 | ZC3H14 | 8.53E−19 | 0.313 | 0.528 | 0.105 | 8.417E−15 |
| RBM39 | 5.15E−16 | −0.468 | 0.731 | 0.589 | 5.080E−12 | RSRC1 | 9.43E−19 | 0.264 | 0.509 | 0.105 | 9.311E−15 |
| SMC6 | 7.86E−16 | −0.288 | 0.407 | 0.202 | 7.758E−12 | CCT4 | 1.20E−18 | 0.357 | 0.685 | 0.214 | 1.185E−14 |
| HSPH1 | 1.15E−15 | −0.280 | 0.38 | 0.198 | 1.139E−11 | NDUFA1 | 1.36E−18 | 0.590 | 0.824 | 0.306 | 1.340E−14 |
| CCNL1 | 1.31E−15 | −0.428 | 0.509 | 0.339 | 1.291E−11 | MRPS35 | 1.86E−18 | 0.640 | 0.694 | 0.202 | 1.834E−14 |
| PHF3 | 1.71E−15 | −0.338 | 0.435 | 0.286 | 1.686E−11 | RNF114 | 2.28E−18 | 0.333 | 0.472 | 0.073 | 2.249E−14 |
| RPL22 | 2.00E−15 | −0.450 | 0.472 | 0.391 | 1.976E−11 | PGRMC1 | 2.63E−18 | 0.343 | 0.583 | 0.141 | 2.591E−14 |
| NEAT1 | 2.63E−15 | −1.486 | 0.13 | 0.512 | 2.594E−11 | MDH2 | 2.68E−18 | 0.273 | 0.565 | 0.145 | 2.645E−14 |
| GALNT1 | 2.84E−15 | −0.294 | 0.398 | 0.246 | 2.808E−11 | KARS | 2.77E−18 | 0.296 | 0.528 | 0.113 | 2.734E−14 |
| DDX17 | 3.41E−15 | −0.309 | 0.741 | 0.512 | 3.364E−11 | MOCS2 | 3.15E−18 | 0.361 | 0.537 | 0.101 | 3.106E−14 |
| ARGLU1 | 3.68E−15 | −0.277 | 0.5 | 0.31 | 3.628E−11 | ROMO1 | 4.37E−18 | 0.481 | 0.778 | 0.278 | 4.309E−14 |
| RPL3 | 5.09E−15 | −0.602 | 0.704 | 0.665 | 5.019E−11 | SPCS1 | 5.09E−18 | 0.502 | 0.583 | 0.125 | 5.024E−14 |
| TPR | 6.59E−15 | −0.398 | 0.602 | 0.431 | 6.503E−11 | TMEM66 | 5.69E−18 | 0.263 | 0.657 | 0.226 | 5.611E−14 |
| RPS15A | 1.05E−14 | −0.835 | 0.852 | 0.919 | 1.035E−10 | ANP32B | 7.26E−18 | 0.425 | 0.704 | 0.226 | 7.160E−14 |
| EIF2S2 | 1.13E−14 | −0.361 | 0.528 | 0.367 | 1.113E−10 | DPY30 | 7.43E−18 | 0.386 | 0.657 | 0.194 | 7.332E−14 |
| MT-ND4L | 1.51E−14 | −0.897 | 0.315 | 0.46 | 1.488E−10 | DNAJC1 | 7.81E−18 | 0.493 | 0.565 | 0.117 | 7.711E−14 |
| MYH9 | 2.23E−14 | −0.397 | 0.491 | 0.363 | 2.203E−10 | ATP5A1 | 8.96E−18 | 0.284 | 0.787 | 0.339 | 8.838E−14 |
| KRT8 | 2.62E−14 | −0.342 | 0.426 | 0.306 | 2.586E−10 | PARP1 | 1.01E−17 | 0.334 | 0.537 | 0.121 | 9.994E−14 |
| FOS | 2.88E−14 | −1.052 | 0.5 | 0.661 | 2.846E−10 | SLC25A3 | 1.02E−17 | 0.424 | 0.648 | 0.181 | 1.011E−13 |
| MORF4L1 | 3.21E−14 | −0.291 | 0.435 | 0.242 | 3.173E−10 | SRSF5 | 1.40E−17 | 0.662 | 0.787 | 0.29 | 1.384E−13 |
| UBC | 3.49E−14 | −0.514 | 0.722 | 0.621 | 3.439E−10 | PNO1 | 1.44E−17 | 0.293 | 0.454 | 0.073 | 1.425E−13 |
| NACA | 5.09E−14 | −0.301 | 0.815 | 0.621 | 5.028E−10 | SIVA1 | 1.56E−17 | 0.330 | 0.574 | 0.153 | 1.544E−13 |
| BPTF | 5.23E−14 | −0.409 | 0.481 | 0.282 | 5.164E−10 | NOL7 | 1.57E−17 | 0.283 | 0.769 | 0.327 | 1.551E−13 |
| SRRM2 | 5.66E−14 | −0.443 | 0.546 | 0.407 | 5.589E−10 | TOP1 | 1.58E−17 | 0.350 | 0.704 | 0.242 | 1.557E−13 |
| JUN | 5.98E−14 | −0.663 | 0.556 | 0.508 | 5.902E−10 | SSR1 | 1.65E−17 | 0.296 | 0.611 | 0.185 | 1.629E−13 |
| RPL36 | 7.31E−14 | −0.664 | 0.741 | 0.778 | 7.210E−10 | SCGB2A2 | 1.68E−17 | 0.299 | 0.213 | 0.629 | 1.655E−13 |
| RPL10A | 1.83E−13 | −0.675 | 0.769 | 0.77 | 1.809E−09 | C1orf43 | 1.77E−17 | 0.307 | 0.657 | 0.218 | 1.748E−13 |
| SLC25A36 | 2.00E−13 | −0.337 | 0.352 | 0.23 | 1.975E−09 | PSMA7 | 1.80E−17 | 0.524 | 0.824 | 0.327 | 1.781E−13 |
| CSTB | 2.15E−13 | −1.082 | 0.454 | 0.524 | 2.118E−09 | SNX6 | 1.92E−17 | 0.258 | 0.62 | 0.194 | 1.898E−13 |
| MT-ND4 | 2.56E−13 | −0.847 | 0.815 | 0.835 | 2.523E−09 | TUFM | 2.20E−17 | 0.264 | 0.509 | 0.121 | 2.175E−13 |
| BDP1 | 2.93E−13 | −0.600 | 0.389 | 0.355 | 2.894E−09 | PSMB8 | 2.27E−17 | 0.281 | 0.407 | 0.056 | 2.239E−13 |
| RPS27A | 4.20E−13 | −0.751 | 0.926 | 0.948 | 4.141E−09 | NDUFB2 | 2.66E−17 | 0.368 | 0.787 | 0.327 | 2.622E−13 |
| TCEA1 | 4.21E−13 | −0.262 | 0.25 | 0.169 | 4.159E−09 | TTC1 | 2.67E−17 | 0.321 | 0.537 | 0.125 | 2.639E−13 |
| GOLGB1 | 5.98E−13 | −0.608 | 0.694 | 0.552 | 5.902E−09 | SNRPD2 | 2.70E−17 | 0.854 | 0.907 | 0.508 | 2.668E−13 |
| LUC7L3 | 6.31E−13 | −0.364 | 0.509 | 0.359 | 6.230E−09 | THOC7 | 2.98E−17 | 0.578 | 0.63 | 0.161 | 2.938E−13 |
| RB1CC1 | 7.71E−13 | −0.303 | 0.444 | 0.278 | 7.612E−09 | SMARCB1 | 3.86E−17 | 0.329 | 0.417 | 0.052 | 3.813E−13 |
| BTG1 | 8.16E−13 | −0.416 | 0.278 | 0.286 | 8.057E−09 | PARK7 | 3.99E−17 | 0.377 | 0.676 | 0.222 | 3.940E−13 |
| SNRPG | 8.34E−13 | −0.355 | 0.139 | 0.165 | 8.232E−09 | NDUFC1 | 5.71E−17 | 0.316 | 0.574 | 0.157 | 5.636E−13 |
| PRRC2C | 8.50E−13 | −0.306 | 0.769 | 0.54 | 8.386E−09 | AP2M1 | 6.47E−17 | 0.288 | 0.519 | 0.117 | 6.381E−13 |
| NUPR1 | 9.99E−13 | −0.779 | 0.37 | 0.347 | 9.855E−09 | STAT1 | 6.50E−17 | 0.837 | 0.778 | 0.29 | 6.414E−13 |
| COX16 | 1.24E−12 | −0.300 | 0.417 | 0.282 | 1.228E−08 | PSME2 | 7.52E−17 | 0.254 | 0.62 | 0.206 | 7.424E−13 |
| RPS13 | 1.44E−12 | −0.453 | 0.926 | 0.81 | 1.422E−08 | ATP5G3 | 8.56E−17 | 0.488 | 0.75 | 0.266 | 8.448E−13 |
| RN7SL2 | 1.58E−12 | −0.323 | 0.269 | 0.198 | 1.558E−08 | SSRP1 | 1.00E−16 | 0.253 | 0.472 | 0.097 | 9.914E−13 |
| PPP1R12A | 1.69E−12 | −0.254 | 0.306 | 0.145 | 1.667E−08 | MRPL35 | 1.10E−16 | 0.287 | 0.444 | 0.081 | 1.085E−12 |
| ANKRD30B | 1.70E−12 | −0.640 | 0.63 | 0.556 | 1.675E−08 | PDIA6 | 1.11E−16 | 0.413 | 0.769 | 0.294 | 1.091E−12 |
| RPL37 | 1.77E−12 | −0.715 | 0.935 | 0.992 | 1.749E−08 | GARS | 1.13E−16 | 0.283 | 0.491 | 0.113 | 1.112E−12 |
| UBE2R2 | 2.31E−12 | −0.293 | 0.167 | 0.125 | 2.279E−08 | C8orf59 | 1.15E−16 | 0.267 | 0.722 | 0.286 | 1.139E−12 |
| CTDSP2 | 2.86E−12 | −0.288 | 0.417 | 0.331 | 2.823E−08 | UCHL5 | 1.48E−16 | 0.299 | 0.444 | 0.077 | 1.464E−12 |
| ASH1L | 4.84E−12 | −0.251 | 0.417 | 0.242 | 4.781E−08 | TMEM97 | 1.53E−16 | 0.454 | 0.5 | 0.085 | 1.508E−12 |
| HMGN2 | 7.14E−12 | −0.382 | 0.315 | 0.206 | 7.042E−08 | SEC61B | 1.55E−16 | 0.437 | 0.62 | 0.173 | 1.534E−12 |
| DYNC1H1 | 7.52E−12 | −0.344 | 0.361 | 0.266 | 7.423E−08 | ACTR2 | 1.78E−16 | 0.279 | 0.667 | 0.25 | 1.755E−12 |
| MT-CYB | 8.71E−12 | −0.420 | 0.75 | 0.653 | 8.594E−08 | 15-Sep | 1.89E−16 | 0.335 | 0.648 | 0.21 | 1.861E−12 |
| RPL21 | 9.82E−12 | −0.444 | 0.139 | 0.198 | 9.692E−08 | RAP1A | 1.90E−16 | 0.337 | 0.426 | 0.056 | 1.875E−12 |
| OXR1 | 1.03E−11 | −0.291 | 0.231 | 0.161 | 1.012E−07 | NDUFB7 | 1.97E−16 | 0.261 | 0.713 | 0.286 | 1.948E−12 |
| NKTR | 1.06E−11 | −0.423 | 0.306 | 0.234 | 1.048E−07 | MT-TP | 2.18E−16 | 1.540 | 0.583 | 0.177 | 2.147E−12 |
| RBM25 | 1.44E−11 | −0.465 | 0.602 | 0.464 | 1.425E−07 | ANAPC5 | 2.34E−16 | 0.352 | 0.5 | 0.093 | 2.308E−12 |
| IRF2BP2 | 1.49E−11 | −0.448 | 0.463 | 0.419 | 1.471E−07 | TMED4 | 2.36E−16 | 0.350 | 0.491 | 0.097 | 2.332E−12 |
| CREBBP | 1.66E−11 | −0.271 | 0.13 | 0.117 | 1.638E−07 | TCP1 | 2.61E−16 | 0.319 | 0.519 | 0.141 | 2.578E−12 |
| ZFAND5 | 1.85E−11 | −0.270 | 0.315 | 0.222 | 1.823E−07 | CTSH | 3.35E−16 | 0.403 | 0.648 | 0.214 | 3.304E−12 |
| MGEA5 | 2.36E−11 | −0.318 | 0.213 | 0.165 | 2.327E−07 | REEP5 | 3.52E−16 | 0.358 | 0.528 | 0.121 | 3.471E−12 |
| PHIP | 2.46E−11 | −0.394 | 0.417 | 0.31 | 2.428E−07 | EZR | 3.96E−16 | 0.272 | 0.62 | 0.206 | 3.908E−12 |
| TRIBI | 2.48E−11 | −0.591 | 0.269 | 0.387 | 2.444E−07 | COX8A | 4.44E−16 | 0.271 | 0.694 | 0.258 | 4.378E−12 |
| RBM3 | 2.84E−11 | −0.320 | 0.731 | 0.54 | 2.805E−07 | UBL5 | 4.65E−16 | 0.742 | 0.852 | 0.395 | 4.587E−12 |
| SNHG9 | 3.42E−11 | −0.263 | 0.213 | 0.157 | 3.380E−07 | POLR2F | 4.76E−16 | 0.332 | 0.574 | 0.153 | 4.697E−12 |
| PNISR | 3.45E−11 | −0.324 | 0.444 | 0.302 | 3.400E−07 | H2AFZ | 5.16E−16 | 0.580 | 0.602 | 0.185 | 5.093E−12 |
| ZNF292 | 3.73E−11 | −0.462 | 0.287 | 0.242 | 3.686E−07 | FJX1 | 5.17E−16 | 1.605 | 0.5 | 0.165 | 5.103E−12 |
| ANXA2 | 3.88E−11 | −0.308 | 0.537 | 0.399 | 3.831E−07 | TMED10 | 5.90E−16 | 0.288 | 0.639 | 0.218 | 5.824E−12 |
| RSRC2 | 4.09E−11 | −0.265 | 0.352 | 0.214 | 4.038E−07 | EBNA1BP2 | 6.91E−16 | 0.344 | 0.593 | 0.169 | 6.822E−12 |
| MIF | 4.35E−11 | −0.635 | 0 | 0.254 | 4.295E−07 | SSBP1 | 6.92E−16 | 0.260 | 0.704 | 0.286 | 6.832E−12 |
| PLIN2 | 4.37E−11 | −0.613 | 0.269 | 0.202 | 4.311E−07 | PAPOLA | 7.15E−16 | 0.416 | 0.778 | 0.315 | 7.060E−12 |
| ANKRD36C | 6.03E−11 | −1.134 | 0.38 | 0.577 | 5.951E−07 | PPIB | 7.39E−16 | 0.587 | 0.574 | 0.173 | 7.291E−12 |
| STXBP1 | 1.21E−10 | −0.521 | 0.231 | 0.286 | 1.192E−06 | PPP1CB | 7.44E−16 | 0.303 | 0.574 | 0.173 | 7.342E−12 |
| BOD1L1 | 1.45E−10 | −0.304 | 0.278 | 0.185 | 1.432E−06 | LAP3 | 1.05E−15 | 0.276 | 0.537 | 0.149 | 1.041E−11 |
| UBE2G2 | 1.46E−10 | −0.296 | 0.231 | 0.185 | 1.441E−06 | COPS2 | 1.09E−15 | 0.292 | 0.454 | 0.093 | 1.073E−11 |
| DICER1 | 1.54E−10 | −0.438 | 0.278 | 0.246 | 1.525E−06 | RHOA | 1.16E−15 | 0.325 | 0.657 | 0.226 | 1.142E−11 |
| KTN1 | 1.60E−10 | −0.330 | 0.769 | 0.581 | 1.577E−06 | EIF3D | 1.86E−15 | 0.255 | 0.639 | 0.234 | 1.840E−11 |
| MTRNR2L12 | 1.71E−10 | −0.731 | 0.093 | 0.319 | 1.688E−06 | CCT5 | 2.00E−15 | 0.538 | 0.787 | 0.315 | 1.973E−11 |
| ERO1L | 1.99E−10 | −0.254 | 0.157 | 0.109 | 1.969E−06 | CALM1 | 2.15E−15 | 0.298 | 0.852 | 0.419 | 2.121E−11 |
| PVT1 | 2.23E−10 | −0.747 | 0.407 | 0.476 | 2.204E−06 | RPN2 | 2.20E−15 | 0.311 | 0.704 | 0.274 | 2.168E−11 |
| RPL10 | 2.31E−10 | −0.468 | 0.148 | 0.234 | 2.279E−06 | GTF3A | 2.23E−15 | 0.379 | 0.583 | 0.165 | 2.199E−11 |
| TMSB10 | 2.80E−10 | −0.481 | 0.759 | 0.681 | 2.764E−06 | MDH1 | 2.35E−15 | 0.280 | 0.546 | 0.169 | 2.316E−11 |
| CHD9 | 2.95E−10 | −0.278 | 0.417 | 0.226 | 2.912E−06 | CCT2 | 2.51E−15 | 0.536 | 0.676 | 0.218 | 2.473E−11 |
| RPS28 | 3.16E−10 | −0.563 | 0.843 | 0.823 | 3.118E−06 | AIMP1 | 3.25E−15 | 0.348 | 0.537 | 0.137 | 3.205E−11 |
| HES1 | 3.85E−10 | −0.421 | 0.389 | 0.363 | 3.797E−06 | RAN | 3.30E−15 | 0.336 | 0.694 | 0.262 | 3.258E−11 |
| RPL18A | 4.25E−10 | −0.259 | 0.185 | 0.137 | 4.191E−06 | EIF4A2 | 4.47E−15 | 0.337 | 0.769 | 0.327 | 4.409E−11 |
| S100A9 | 6.28E−10 | −1.004 | 0 | 0.23 | 6.200E−06 | SRSF2 | 5.98E−15 | 0.321 | 0.583 | 0.173 | 5.906E−11 |
| KMT2C | 7.25E−10 | −0.329 | 0.333 | 0.246 | 7.151E−06 | HDLBP | 7.32E−15 | 0.330 | 0.685 | 0.258 | 7.225E−11 |
| MT-ND2 | 1.09E−09 | −0.456 | 0.778 | 0.742 | 1.073E−05 | HDGF | 7.92E−15 | 0.350 | 0.722 | 0.302 | 7.814E−11 |
| SLK | 1.25E−09 | −0.351 | 0.296 | 0.202 | 1.230E−05 | HSPA5 | 7.93E−15 | 0.743 | 0.704 | 0.278 | 7.824E−11 |
| NSD1 | 1.34E−09 | −0.267 | 0.231 | 0.169 | 1.325E−05 | CNIH1 | 9.06E−15 | 0.418 | 0.667 | 0.222 | 8.937E−11 |
| TRPS1 | 1.42E−09 | −0.358 | 0.639 | 0.516 | 1.402E−05 | TRAM1 | 1.05E−14 | 0.283 | 0.63 | 0.222 | 1.037E−10 |
| SUB1 | 1.64E−09 | −0.280 | 0.889 | 0.718 | 1.619E−05 | PRDX2 | 1.21E−14 | 0.334 | 0.667 | 0.238 | 1.191E−10 |
| RPL4 | 3.49E−09 | −0.340 | 0.815 | 0.698 | 3.443E−05 | MSMO1 | 1.24E−14 | 0.252 | 0.509 | 0.145 | 1.227E−10 |
| MT-ND5 | 3.61E−09 | −0.507 | 0.481 | 0.423 | 3.567E−05 | SRP72 | 1.42E−14 | 0.430 | 0.639 | 0.21 | 1.401E−10 |
| MTRNR2L8 | 3.91E−09 | −0.619 | 0.185 | 0.278 | 3.862E−05 | UQCRQ | 1.57E−14 | 0.536 | 0.815 | 0.359 | 1.550E−10 |
| CHD2 | 3.96E−09 | −0.521 | 0.296 | 0.29 | 3.906E−05 | BUD31 | 1.62E−14 | 0.280 | 0.444 | 0.093 | 1.596E−10 |
| UTRN | 4.17E−09 | −0.272 | 0.426 | 0.262 | 4.113E−05 | ACP1 | 1.62E−14 | 0.355 | 0.667 | 0.238 | 1.601E−10 |
| MT-ATP8 | 4.59E−09 | −0.495 | 0.389 | 0.407 | 4.534E−05 | NDUFAF6 | 1.67E−14 | 0.267 | 0.546 | 0.161 | 1.647E−10 |
| ADNP | 4.93E−09 | −0.300 | 0.213 | 0.145 | 4.865E−05 | RARRES3 | 1.80E−14 | 0.687 | 0.528 | 0.121 | 1.779E−10 |
| ZC3H11A | 4.99E−09 | −0.258 | 0.38 | 0.25 | 4.921E−05 | NUCB2 | 1.81E−14 | 0.318 | 0.546 | 0.165 | 1.790E−10 |
| EGR1 | 5.33E−09 | −0.629 | 0.352 | 0.419 | 5.256E−05 | APIP | 1.86E−14 | 1.829 | 0.37 | 0.069 | 1.839E−10 |
| IL8 | 5.50E−09 | −1.052 | 0 | 0.21 | 5.424E−05 | CCT6A | 1.97E−14 | 0.472 | 0.824 | 0.375 | 1.940E−10 |
| CITED2 | 7.42E−09 | −0.386 | 0.343 | 0.278 | 7.319E−05 | DPM2 | 2.02E−14 | 0.892 | 0.824 | 0.556 | 1.998E−10 |
| PBX1 | 7.49E−09 | −0.321 | 0.463 | 0.335 | 7.395E−05 | LMAN1 | 2.22E−14 | 0.430 | 0.648 | 0.238 | 2.192E−10 |
| MLPH | 8.23E−09 | −0.355 | 0.204 | 0.198 | 8.123E−05 | SLC38A1 | 2.74E−14 | 0.419 | 0.546 | 0.145 | 2.702E−10 |
| RPS15 | 1.46E−08 | −0.493 | 0.861 | 0.839 | 1.437E−04 | NDUFA13 | 2.99E−14 | 0.570 | 0.787 | 0.335 | 2.956E−10 |
| MT-ND6 | 1.69E−08 | −0.252 | 0.111 | 0.109 | 1.668E−04 | MAGOHB | 3.35E−14 | 0.298 | 0.296 | 0.016 | 3.306E−10 |
| MTRNR2L2 | 1.94E−08 | −0.621 | 0.139 | 0.294 | 1.917E−04 | GBP4 | 3.62E−14 | 0.640 | 0.241 | 0.008 | 3.574E−10 |
| SYNE2 | 2.05E−08 | −0.358 | 0.565 | 0.423 | 2.026E−04 | NME1 | 3.91E−14 | 0.251 | 0.537 | 0.169 | 3.863E−10 |
| ACTB | 2.76E−08 | −0.397 | 0.704 | 0.641 | 2.725E−04 | VPS26A | 4.08E−14 | 0.257 | 0.389 | 0.073 | 4.029E−10 |
| IRX2 | 3.45E−08 | −0.314 | 0.13 | 0.161 | 3.406E−04 | DECR1 | 4.15E−14 | 0.287 | 0.407 | 0.077 | 4.098E−10 |
| SREK1 | 3.72E−08 | −0.296 | 0.398 | 0.282 | 3.673E−04 | UBB | 4.35E−14 | 0.614 | 0.824 | 0.379 | 4.297E−10 |
| AHNAK | 4.31E−08 | −0.345 | 0.194 | 0.169 | 4.254E−04 | MCM4 | 4.70E−14 | 0.338 | 0.398 | 0.065 | 4.639E−10 |
| PET100 | 4.57E−08 | −0.399 | 0.278 | 0.238 | 4.509E−04 | USP1 | 6.89E−14 | 0.291 | 0.574 | 0.19 | 6.803E−10 |
| BAZ2B | 4.81E−08 | −0.449 | 0.343 | 0.315 | 4.747E−04 | GPBP1 | 8.50E−14 | 0.254 | 0.556 | 0.206 | 8.393E−10 |
| MUM1 | 4.86E−08 | −0.260 | 0.102 | 0.105 | 4.792E−04 | FAM98B | 8.75E−14 | 0.361 | 0.361 | 0.04 | 8.632E−10 |
| NFKBIZ | 5.54E−08 | −0.352 | 0.185 | 0.194 | 5.467E−04 | TXNL1 | 9.46E−14 | 0.261 | 0.5 | 0.145 | 9.337E−10 |
| ZFAS1 | 6.22E−08 | −0.268 | 0.704 | 0.556 | 6.134E−04 | MED10 | 9.67E−14 | 0.281 | 0.454 | 0.101 | 9.539E−10 |
| CRIM1 | 6.37E−08 | −0.272 | 0.213 | 0.149 | 6.288E−04 | NOP16 | 1.06E−13 | 0.306 | 0.38 | 0.06 | 1.043E−09 |
| RPS2 | 7.80E−08 | −0.466 | 0.815 | 0.802 | 7.697E−04 | NDUFS1 | 1.06E−13 | 0.257 | 0.509 | 0.149 | 1.047E−09 |
| ANKRD36 | 1.29E−07 | −0.750 | 0.083 | 0.286 | 1.274E−03 | SSR4 | 1.26E−13 | 0.416 | 0.519 | 0.137 | 1.246E−09 |
| S100A8 | 1.59E−07 | −0.863 | 0 | 0.177 | 1.573E−03 | CHCHD2 | 1.29E−13 | 0.322 | 0.75 | 0.339 | 1.277E−09 |
| RPL13A | 2.13E−07 | −0.360 | 0.852 | 0.778 | 2.101E−03 | SLC25A6 | 1.34E−13 | 0.438 | 0.62 | 0.198 | 1.326E−09 |
| TRIM44 | 2.89E−07 | −0.425 | 0.528 | 0.609 | 2.857E−03 | XAF1 | 1.46E−13 | 0.611 | 0.454 | 0.121 | 1.444E−09 |
| ZFP36L1 | 3.56E−07 | −0.364 | 0.713 | 0.661 | 3.516E−03 | MGP | 1.49E−13 | 0.535 | 0.833 | 0.98 | 1.471E−09 |
| RPS20 | 4.75E−07 | −0.554 | 0.907 | 0.935 | 4.686E−03 | DNAJA1 | 1.71E−13 | 0.368 | 0.861 | 0.44 | 1.692E−09 |
| RN7SL851P | 5.46E−07 | −3.592 | 0 | 0.165 | 5.388E−03 | ACTR6 | 1.83E−13 | 0.265 | 0.389 | 0.077 | 1.811E−09 |
| FOSB | 7.51E−07 | −0.729 | 0.185 | 0.351 | 7.410E−03 | PSMA3 | 1.88E−13 | 0.287 | 0.694 | 0.29 | 1.860E−09 |
| ZBTB43 | 7.98E−07 | −0.557 | 0.435 | 0.524 | 7.875E−03 | MLEC | 2.01E−13 | 0.318 | 0.491 | 0.125 | 1.986E−09 |
| KIAA1109 | 8.66E−07 | −0.260 | 0.139 | 0.141 | 8.543E−03 | MAPKAP1 | 2.25E−13 | 1.000 | 0.88 | 0.762 | 2.225E−09 |
| EDN1 | 1.09E−06 | −0.701 | 0.213 | 0.327 | 1.078E−02 | HLA-E | 2.27E−13 | 0.620 | 0.676 | 0.282 | 2.243E−09 |
| MTRNR2L3 | 1.13E−06 | −0.646 | 0.231 | 0.331 | 1.116E−02 | CYP27B1 | 2.34E−13 | 0.292 | 0.37 | 0.052 | 2.313E−09 |
| NCL | 1.26E−06 | −0.287 | 0.898 | 0.774 | 1.243E−02 | CP | 2.56E−13 | 1.200 | 0.463 | 0.649 | 2.529E−09 |
| AC013394.2 | 1.30E−06 | −0.261 | 0.056 | 0.101 | 1.279E−02 | SEC23B | 2.95E−13 | 0.282 | 0.407 | 0.077 | 2.908E−09 |
| MDM4 | 1.41E−06 | −0.254 | 0.194 | 0.153 | 1.395E−02 | IFNAR1 | 2.99E−13 | 0.268 | 0.454 | 0.113 | 2.953E−09 |
| DUSP1 | 1.56E−06 | −0.421 | 0.083 | 0.177 | 1.539E−02 | FRA10AC1 | 3.22E−13 | 0.307 | 0.37 | 0.113 | 3.182E−09 |
| SDCBP | 2.01E−06 | −0.259 | 0.194 | 0.19 | 1.979E−02 | PDCD5 | 3.32E−13 | 0.347 | 0.667 | 0.258 | 3.280E−09 |
| RPL26 | 3.74E−06 | −0.514 | 0.963 | 0.988 | 3.695E−02 | MRPL13 | 3.39E−13 | 0.265 | 0.537 | 0.177 | 3.342E−09 |
| C8orf4 | 3.91E−06 | −0.551 | 0.12 | 0.177 | 3.856E−02 | IFIH1 | 3.79E−13 | 0.404 | 0.565 | 0.173 | 3.741E−09 |
| TBX3 | 4.09E−06 | −0.364 | 0 | 0.145 | 4.035E−02 | ACTR3 | 4.48E−13 | 0.370 | 0.676 | 0.262 | 4.418E−09 |
| CDK9 | 5.05E−06 | −0.255 | 0.222 | 0.202 | 4.989E−02 | MSRB1 | 4.70E−13 | 0.262 | 0.241 | 0.004 | 4.643E−09 |
| | | | | | | PDIA3 | 5.44E−13 | 0.474 | 0.759 | 0.343 | 5.367E−09 |
| | | | | | | UBE2J1 | 5.57E−13 | 0.357 | 0.426 | 0.085 | 5.502E−09 |
| | | | | | | XRCC5 | 5.69E−13 | 0.313 | 0.694 | 0.302 | 5.614E−09 |
| | | | | | | CCT3 | 6.96E−13 | 0.440 | 0.796 | 0.375 | 6.869E−09 |
| | | | | | | BRIX1 | 7.02E−13 | 0.290 | 0.602 | 0.222 | 6.927E−09 |
| | | | | | | TSPO | 7.42E−13 | 0.282 | 0.648 | 0.262 | 7.319E−09 |
| | | | | | | PDIA4 | 8.22E−13 | 0.329 | 0.509 | 0.141 | 8.112E−09 |
| | | | | | | TMBIM6 | 8.30E−13 | 0.315 | 0.741 | 0.339 | 8.187E−09 |
| | | | | | | CD46 | 8.77E−13 | 0.301 | 0.741 | 0.367 | 8.653E−09 |
| | | | | | | EIF5 | 9.40E−13 | 0.357 | 0.75 | 0.335 | 9.273E−09 |
| | | | | | | IFI27 | 1.12E−12 | 0.601 | 0.343 | 0.04 | 1.104E−08 |
| | | | | | | SNRPB | 1.19E−12 | 0.348 | 0.546 | 0.165 | 1.178E−08 |
| | | | | | | SMARCA5 | 1.27E−12 | 0.431 | 0.676 | 0.258 | 1.250E−08 |
| | | | | | | HLA-C | 1.40E−12 | 0.324 | 0.778 | 0.375 | 1.385E−08 |
| | | | | | | NOP56 | 1.53E−12 | 0.596 | 0.639 | 0.222 | 1.513E−08 |
| | | | | | | MT-TF | 1.64E−12 | 0.323 | 0.324 | 0.069 | 1.620E−08 |
| | | | | | | VBP1 | 1.64E−12 | 0.254 | 0.481 | 0.141 | 1.622E−08 |
| | | | | | | SEC62 | 1.77E−12 | 0.375 | 0.694 | 0.294 | 1.745E−08 |
| | | | | | | GSPT1 | 2.05E−12 | 0.358 | 0.657 | 0.258 | 2.022E−08 |
| | | | | | | SEC11C | 2.29E−12 | 0.502 | 0.611 | 0.242 | 2.261E−08 |
| | | | | | | MTHFD2 | 2.33E−12 | 0.252 | 0.574 | 0.23 | 2.298E−08 |
| | | | | | | LAMP2 | 3.53E−12 | 0.290 | 0.602 | 0.242 | 3.483E−08 |
| | | | | | | ERP29 | 3.73E−12 | 0.291 | 0.352 | 0.06 | 3.678E−08 |
| | | | | | | ME1 | 4.23E−12 | 0.280 | 0.519 | 0.169 | 4.171E−08 |
| | | | | | | HNRNPA2B1 | 4.46E−12 | 0.466 | 0.889 | 0.5 | 4.400E−08 |
| | | | | | | EPRS | 4.46E−12 | 0.312 | 0.722 | 0.331 | 4.402E−08 |
| | | | | | | NDUFB1 | 4.53E−12 | 0.290 | 0.787 | 0.391 | 4.472E−08 |
| | | | | | | MRPL48 | 9.30E−12 | 0.330 | 0.324 | 0.036 | 9.175E−08 |
| | | | | | | RARRES1 | 1.07E−11 | 0.986 | 0.62 | 0.556 | 1.053E−07 |
| | | | | | | MARS | 1.26E−11 | 0.252 | 0.296 | 0.028 | 1.239E−07 |
| | | | | | | EIF2S3 | 1.41E−11 | 0.359 | 0.454 | 0.117 | 1.396E−07 |
| | | | | | | FAM98A | 1.42E−11 | 0.303 | 0.296 | 0.028 | 1.398E−07 |
| | | | | | | MAGED2 | 1.44E−11 | 0.253 | 0.481 | 0.161 | 1.421E−07 |
| | | | | | | LSM7 | 1.46E−11 | 0.253 | 0.593 | 0.266 | 1.437E−07 |
| | | | | | | TMPO | 1.59E−11 | 0.300 | 0.352 | 0.065 | 1.567E−07 |
| | | | | | | CNBP | 1.73E−11 | 0.274 | 0.787 | 0.419 | 1.708E−07 |
| | | | | | | TMEM59 | 2.14E−11 | 0.275 | 0.63 | 0.274 | 2.109E−07 |
| | | | | | | BUB3 | 2.16E−11 | 0.359 | 0.417 | 0.093 | 2.136E−07 |
| | | | | | | EIF5B | 2.22E−11 | 0.259 | 0.796 | 0.419 | 2.190E−07 |
| | | | | | | PPM1G | 2.27E−11 | 0.279 | 0.491 | 0.157 | 2.237E−07 |
| | | | | | | STMN1 | 3.52E−11 | 0.256 | 0.481 | 0.185 | 3.470E−07 |
| | | | | | | TUBA1B | 3.60E−11 | 0.443 | 0.546 | 0.202 | 3.552E−07 |
| | | | | | | GBP1 | 3.63E−11 | 0.763 | 0.667 | 0.298 | 3.579E−07 |
| | | | | | | PAICS | 4.83E−11 | 0.287 | 0.509 | 0.165 | 4.767E−07 |
| | | | | | | AARD | 5.35E−11 | 0.975 | 0.361 | 0.069 | 5.281E−07 |
| | | | | | | SH3BGRL | 5.62E−11 | 0.264 | 0.611 | 0.266 | 5.543E−07 |
| | | | | | | SLC25A5 | 6.10E−11 | 0.302 | 0.648 | 0.278 | 6.015E−07 |
| | | | | | | RPL27 | 8.23E−11 | 0.733 | 0.944 | 0.754 | 8.122E−07 |
| | | | | | | CD74 | 1.46E−10 | 0.942 | 0.491 | 0.246 | 1.445E−06 |
| | | | | | | CANX | 2.00E−10 | 0.353 | 0.796 | 0.419 | 1.976E−06 |
| | | | | | | APOL6 | 2.26E−10 | 0.682 | 0.713 | 0.383 | 2.229E−06 |
| | | | | | | PEBP1 | 2.95E−10 | 0.383 | 0.713 | 0.347 | 2.910E−06 |
| | | | | | | OAZ1 | 3.22E−10 | 0.413 | 0.815 | 0.435 | 3.181E−06 |
| | | | | | | PARP9 | 3.43E−10 | 0.436 | 0.556 | 0.214 | 3.389E−06 |
| | | | | | | SEPP1 | 4.28E−10 | 0.927 | 0.648 | 0.665 | 4.224E−06 |
| | | | | | | MT2A | 4.91E−10 | 0.323 | 0.648 | 0.306 | 4.847E−06 |
| | | | | | | C14orf1 | 5.08E−10 | 0.280 | 0.343 | 0.081 | 5.013E−06 |
| | | | | | | PCNA | 8.35E−10 | 0.418 | 0.315 | 0.052 | 8.241E−06 |
| | | | | | | SLC39A6 | 8.93E−10 | 0.293 | 0.454 | 0.25 | 8.812E−06 |
| | | | | | | MX1 | 9.70E−10 | 0.272 | 0.417 | 0.129 | 9.571E−06 |
| | | | | | | HLA-A | 1.22E−09 | 0.414 | 0.639 | 0.29 | 1.204E−05 |
| | | | | | | HMGCS1 | 1.25E−09 | 0.365 | 0.694 | 0.387 | 1.231E−05 |
| | | | | | | RPL22L1 | 1.33E−09 | 0.280 | 0.472 | 0.177 | 1.314E−05 |
| | | | | | | FAM173A | 1.56E−09 | 0.262 | 0.25 | 0.024 | 1.537E−05 |
| | | | | | | BST2 | 1.64E−09 | 0.290 | 0.426 | 0.129 | 1.615E−05 |
| | | | | | | RPS21 | 2.07E−09 | 0.557 | 1 | 0.972 | 2.043E−05 |
| | | | | | | CDK4 | 2.41E−09 | 0.760 | 0.787 | 0.54 | 2.379E−05 |
| | | | | | | INSIG1 | 3.32E−09 | 0.520 | 0.694 | 0.419 | 3.274E−05 |
| | | | | | | RPL9 | 3.35E−09 | 0.291 | 0.787 | 0.44 | 3.311E−05 |
| | | | | | | UQCRH | 3.90E−09 | 0.306 | 0.759 | 0.415 | 3.845E−05 |
| | | | | | | PTGES3 | 5.66E−09 | 0.300 | 0.602 | 0.262 | 5.583E−05 |
| | | | | | | EZH2 | 6.18E−09 | 0.297 | 0.25 | 0.04 | 6.104E−05 |
| | | | | | | XBP1 | 1.11E−08 | 0.365 | 0.843 | 0.778 | 1.091E−04 |
| | | | | | | MLF1IP | 1.28E−08 | 0.295 | 0.194 | 0.016 | 1.264E−04 |
| | | | | | | MT-TS2 | 1.29E−08 | 0.828 | 0.278 | 0.056 | 1.276E−04 |
| | | | | | | HSP90AB1 | 1.75E−08 | 0.512 | 0.926 | 0.681 | 1.723E−04 |
| | | | | | | S100A6 | 2.06E−08 | 0.579 | 0.602 | 0.282 | 2.031E−04 |
| | | | | | | APOD | 2.59E−08 | 0.639 | 0.204 | 0.351 | 2.554E−04 |
| | | | | | | XRCC6BP1 | 3.04E−08 | 0.282 | 0.611 | 0.343 | 3.001E−04 |
| | | | | | | HLA-B | 3.55E−08 | 0.563 | 0.889 | 0.706 | 3.508E−04 |
| | | | | | | PKIB | 4.05E−08 | 0.294 | 0.278 | 0.048 | 3.998E−04 |
| | | | | | | NDUFA4 | 5.44E−08 | 0.487 | 0.88 | 0.597 | 5.374E−04 |
| | | | | | | MAN1A2 | 5.52E−08 | 0.362 | 0.741 | 0.435 | 5.444E−04 |
| | | | | | | RSL1D1 | 8.63E−08 | 0.409 | 0.815 | 0.488 | 8.520E−04 |
| | | | | | | TOB1 | 9.03E−08 | 0.369 | 0.639 | 0.444 | 8.907E−04 |
| | | | | | | CD44 | 9.50E−08 | 0.538 | 0.917 | 0.992 | 9.375E−04 |
| | | | | | | RPL8 | 1.15E−07 | 0.438 | 0.972 | 0.863 | 1.138E−03 |
| | | | | | | MED30 | 1.32E−07 | 0.383 | 0.278 | 0.056 | 1.303E−03 |
| | | | | | | IFI44L | 1.34E−07 | 0.633 | 0.481 | 0.206 | 1.325E−03 |
| | | | | | | IFI6 | 1.89E−07 | 0.726 | 0.75 | 0.565 | 1.861E−03 |
| | | | | | | MYC | 2.03E−07 | 0.583 | 0.546 | 0.331 | 2.006E−03 |
| | | | | | | OAS1 | 2.19E−07 | 0.260 | 0.213 | 0.024 | 2.164E−03 |
| | | | | | | HSPD1 | 2.37E−07 | 0.394 | 0.852 | 0.577 | 2.336E−03 |
| | | | | | | PALLD | 2.57E−07 | 0.293 | 0.222 | 0.032 | 2.537E−03 |
| | | | | | | GBP3 | 3.55E−07 | 0.293 | 0.343 | 0.105 | 3.501E−03 |
| | | | | | | UTP23 | 3.58E−07 | 0.498 | 0.944 | 0.931 | 3.533E−03 |
| | | | | | | TSPAN31 | 4.08E−07 | 0.251 | 0.417 | 0.19 | 4.027E−03 |
| | | | | | | IL6ST | 5.29E−07 | 0.319 | 0.38 | 0.218 | 5.216E−03 |
| | | | | | | HSBP1 | 7.55E−07 | 0.357 | 0.676 | 0.359 | 7.450E−03 |
| | | | | | | RPS3 | 9.55E−07 | 0.425 | 0.935 | 0.734 | 9.422E−03 |
| | | | | | | EREG | 1.30E−06 | 0.425 | 0.139 | 0.004 | 1.280E−02 |
| | | | | | | IGJ | 1.68E−06 | 2.263 | 0.111 | 0 | 1.654E−02 |
| | | | | | | RPS6 | 1.77E−06 | 0.477 | 1 | 0.996 | 1.742E−02 |
| | | | | | | FAM46C | 1.85E−06 | 0.319 | 0.148 | 0.008 | 1.829E−02 |
| | | | | | | UGCG | 3.08E−06 | 0.391 | 0.194 | 0.028 | 3.039E−02 |
| | | | | | | RPL7A | 3.13E−06 | 0.311 | 0.889 | 0.641 | 3.094E−02 |
| | | | | | | RPS11 | 3.14E−06 | 0.354 | 1 | 0.96 | 3.099E−02 |
| | | | | | | ATAD2 | 3.35E−06 | 0.254 | 0.259 | 0.069 | 3.307E−02 |

Table 1 shows differential expression results between treated and untreated single cells from the same patient treated and untreated with immunotherapy. The results show genes “up” (up) in post treatment or “down” (dn) in post-treatment. These data have been corrected for multiple sampling and all genes with p-value<0.05 corrected are reported.

Table 2. Shared genes upregulated and downregulated between treated and untreated and genes shared between the responding patients or non responding patients.

The shared3 vs. shared4 refers to genes that were shared in at least 3 of patients 014, 029, 037, and 043 and genes shared in all 4 patients. Patient 001 was excluded from this analysis as only 41 tumor cells total were available for this patient, although this patient does have patient-specific differential expression results.

Blanket description of these genes shows up regulation of cycling and genes known to be involved in antigen presentation going up post-treatment. The other side shows mostly ribosomal genes and some genes associated with coactivation of oxidation/reduction.

Differentially expressed genes between treated and untreated (up or down respectively) which are shared between the responding patients (RvNR) or non responding patients (NRvR). Note—this is not differential expression between responding and non-responding tumor cells. This is differential expression between treated and untreated with results that are shared between responding and non-responding tumor cells.

Blanket description of these genes shows marked up regulation of antigen-presentation-associated machinery.

TABLE 2
Shared between 3 patients
Down
ACTB, AHNAK, BTG1, BZW1, C6orf62, C8orf59, COX6C, DANCR, DUSP1, EEF1B2,
EEF1D, EGR1, EIF2S2, FOS, FTH1, FTL, HES1, HNRNPA1, JUNB, METTL12, MIF,
MORF4L1, MT-ATP6, MT-ATP8, MT-CO1, MT-CO2, MT-CO3, MT-CYB, MT-ND1, MT-
ND2, MT-ND4, MT-ND4L, MT-ND5, MT-ND6, MT-RNR1, MT-TL1, MT-TV,
MTRNR2L1, MTRNR2L11, MTRNR2L12, MTRNR2L2, MTRNR2L3, MTRNR2L8,
NDUFB1, NEAT1, NR4A2, PCBP2, PET100, PFDN5, PPDPF, PRDX2, RB1CC1,
RNA18S5, RNA28S5, RNY1, ROMO1, RPL10, RPL10A, RPL12, RPL13, RPL13A, RPL15,
RPL18, RPL18A, RPL22, RPL23A, RPL26, RPL27A, RPL28, RPL29, RPL31, RPL32,
RPL34, RPL35, RPL36, RPL37, RPL39, RPL4, RPL41, RPL6, RPL7A, RPL8, RPL9, RPN2,
RPS14, RPS15, RPS15A, RPS18, RPS19, RPS2, RPS20, RPS25, RPS27, RPS27A, RPS28,
RPS29, RPS3, RPS3A, RPS4X, RPS7, RPS9, RPSA, S100A8, SLK, SNHG9, SYNGR2,
TMA7, TMSB10, TMSB4X, UBC and UQCRB
Up
ACBD3, ACP1, ACTR2, ACTR3, ACTR6, ADSS, AMD1, AP2M1, APEX1, ARF1, ARPC5,
ASH1L, ATAD2, ATP5G3, B2M, BROX, C1orf21, C1orf43, CALM1, CANX, CAPN2,
CAPRIN1, CCDC59, CCNC, CCT2, CCT3, CCT4, CCT6A, CD109, CD46, CDC16,
CDCA7L, CLTC, CMPK1, CNBP, COA6, COPS2, CP, DENND1B, DHX9, DNAJC3, DST,
DSTN, ECT2, EID1, EIF2AK2, EIF2S1, EIF3D, EIF4A2, EIF5, EMC4, ENO1, EPRS, EZH2,
FKBP3, GADD45GIP1, GMPS, GTF3A, GTPBP4, HADHA, HDGF, HDLBP, HIST1H1D,
HIST1H4C, HNRNPA2B1, HSP90B1, HSPA5, HSPH1, IFNAR1, IQGAP1, LAMP2,
LMAN1, LPP, LRPPRC, MAN1A2, MAPKAP1, MBNL1, MBNL2, MED10, MORF4L2,
MRPL13, MRPL42, MRPS35, MX1, NASP, NOL11, NSMCE2, NUCB2, NUCKS1,
NUDCD1, PAICS, PAPOLA, PARP1, PDCD10, PDHX, PDIA6, PGRMC1, PIK3R1,
PLRG1, PMAIP1, PMP22, POLR2B, PPM1G, PRPF40A, PSMA3, PSME4, PTBP3,
PTTG1IP, RAB10, RAD21, RAN, RARRES1, RHOA, RNF168, RSRC1, S100A6, SAP30BP,
SCRN1, SEC63, SENP6, SF3B1, SLC38A1, SMARCA5, SMC3, SMEK2, SNX6, SPEN,
SRP72, SRSF3, SSR3, SSRP1, ST3GAL1, SUCO, TCP1, TKT, TM4SF1, TMPO, TOMM20,
TOMM70A, TP53BP2, TPM1, TRAM1, TXNL1, UBE2J1, UBE2K, UCHL5, UGP2, USP1,
USP16, VBP1, VIM, XIST, ZC3H14 and ZDHHC20
Shared between 4 patients
Down
COX6C, EEF1D, RNA28S5, RPL10, RPL12, RPL13A, RPL18A, RPL23A, RPL26, RPL36,
RPL41, RPL6, RPS15, RPS15A, RPS18, RPS27, RPS27A, RPS28, RPS29 and RPS7
Up
ACTR3, ARF1, CCT4, CCT6A, COPS2, EIF2AK2, EPRS, HDGF, HIST1H1D, HIST1H4C,
HNRNPA2B1, HSP90B1, LAMP2, MRPL13, PAPOLA, PPM1G, RHOA, SLC38A1,
SMARCA5, SRP72, SSR3, TCP1, TRAM1 and TXNL1
Shared between Non-responders
Down
ACTB, ACTN4, AP2S1, APH1A, ATP5I, ATP5J2, ATP5L, C19orf48, C4orf48, C6orf62,
C8orf59, CDC37, CDKN2A, CEBPB, CFL1, CHCHD2, CHCHD3, CKB, COX5B, COX6A1,
COX6C, COX7A2, COX7B, DANCR, DUSP18, EEF1B2, EEF1D, FAU, FTL, FXYD5,
GPX1, GSTP1, HAP1, HNRNPA1, JUP, LGALS1, LHCGR, LSM4, LSM7, METTL12, MIF,
MLF2, MRPS12, MRPS26, MT-ATP6, MT-ATP8, MT-CO1, MT-CO2, MT-CO3, MT-CYB,
MT-ND1, MT-ND2, MT-ND4, MT-ND4L, MT-ND5, MT-ND6, MT-RNR1, MT-TL1, MT-
TV, MTRNR2L1, MTRNR2L11, MTRNR2L12, MTRNR2L13, MTRNR2L2, MTRNR2L3,
MTRNR2L5, MTRNR2L6, MTRNR2L7, MTRNR2L8, MZT2B, NDUFA11, NDUFA13,
NDUFA2, NDUFB1, NDUFB4, OAZ1, PCBP1, PCBP2, PET100, PFDN5, PFN1, POLR2J,
PPDPF, PRDX2, PSMB3, RAB32, RHOC, RMRP, RNA18S5, RNA28S5, RNY1, ROMO1,
RPL10, RPL12, RPL13, RPL13A, RPL15, RPL18, RPL18A, RPL23A, RPL24, RPL26,
RPL27A, RPL28, RPL29, RPL31, RPL32, RPL34, RPL35, RPL36, RPL36AL, RPL39,
RPL41, RPL6, RPL7A, RPL8, RPL9, RPN2, RPS14, RPS15, RPS15A, RPS18, RPS19,
RPS27, RPS27A, RPS28, RPS29, RPS3, RPS3A, RPS4X, RPS7, RPS9, S100A11, S100A13,
SERF2, SNHG5, SSR4, SYNGR2, TAGLN2, TIMM8B, TMA7, TMSB10, TMSB4X,
TRIM28, TTC19, TUBA1A, TUBA1B, UBA52 and UBL5
Up
ABI2, ACBD3, ACIN1, ACSL4, ACTR3, ADK, ADSS, AMD1, ANKRD17, APEX1, AQR,
ARF1, ARFGEF2, ARHGEF12, ARPC5, ASH1L, ASPH, ATL3, ATP1A1, ATP2A2, BNIP3,
BROX, BRWD1, C1orf21, CAAP1, CANX, CAPN2, CAPRIN1, CBX5, CCDC59,
CCDC90B, CCNC, CCT2, CCT4, CCT6A, CD109, CDC16, CDC5L, CDCA7L, CEBPG,
CHEK1, CHML, CLIC4, CLTC, CMPK1, CNBP, COPB1, COPS2, CTTNBP2NL, CUL5,
DARS, DCBLD1, DENND1B, DHRS7, DHX36, DHX40, DHX9, DICER1, DNAJC10,
DNAJC3, DPH3, DST, DSTN, ECT2, EID1, EIF2AK1, EIF2AK2, EIF2S1, EIF3D, EIF4A2,
EMC4, ENDOD1, ENO1, EPRS, EPS8, ERC1, ERRFI1, ETF1, EXOC3, FAM208B, FAR1,
FNDC3A, FUBP1, G3BP2, GADD45GIP1, GDI2, GLTSCR2, GMPS, GOLGB1, GTPBP4,
H2AFY, H3F3B, HADHA, HDGF, HDLBP, HIST1H1B, HIST1H1C, HIST1H1D,
HIST1H1E, HIST1H4C, HIST2H2AC, HNRNPA2B1, HNRNPUL1, HSP90B1, HSPA5,
HSPH1, IARS, ILF3, INO80D, IQGAP1, ITGA2, KIAA0368, KIAA0947, KIAA1429,
KIF1B, KIF2A, LAMP2, LARP4, LARS, LCOR, LEO1, LPP, LRPPRC, LTN1, MAN1A2,
MAP1LC3B, March7, MARK3, MBD4, MBNL1, MBNL2, MCUR1, MDN1, MGA,
MGEA5, MIB1, MOB1B, MORF4L2, MRPL13, MRPL42, MRPS35, MSANTD3, MYCBP2,
MYSM1, NASP, NCL, NEDD4L, NET1, NFX1, NOL11, NSMCE2, NUCKS1, NUDCD1,
NUDT21, PAIP2, PAPOLA, PBX1, PCYT1A, PDCD10, PDE4DIP, PIK3R1, PIK3R3,
PLRG1, PMAIP1, PMP22, PNPLA8, POLR2B, PPFIA1, PPM1G, PPP2R2D, PPP4R1,
PRPF40A, PSMD2, PSME4, PTBP3, PTPLB, PTPN1, PTTG1IP, RAB10, RAB2A, RABEP1,
RAD21, RBM28, REEP3, RHOA, RNF115, RNF168, SAP30BP, SCRN1, SEC63, SENP2,
SENP6, SETD3, SETX, SF3B1, SFSWAP, SH3GLB1, SLC12A2, SLC38A1, SLC4A7,
SMARCA5, SMARCC1, SMC3, SMEK1, SMEK2, SMNDC1, SNRNP200, SNX4, SOCS4,
SPEN, SRP72, SRRM2, SRSF3, SSR3, SSRP1, ST3GAL1, STK3, SUCO, TAF1D, TAF2,
TCP1, TIMMDC1, TIPARP, TKT, TLK1, TM9SF3, TOMM20, TOMM70A, TP53BP2,
TPM1, TRA2A, TRAM1, TRIP12, TUG1, TXNL1, UAP1, UBA6, UBAP1, UBAP2, UBE2H,
UBE2K, UBTF, UBXN4, UGP2, USP14, USP16, USP34, USP9X, UTP20, VIM, WDR3,
XIST, XRN2, YES1, YWHAH, ZC3H14, ZDHHC20, ZFAS1, ZFYVE16 and ZNF638
Shared between Responders
Down
AHNAK, ANKRD30B, BTG1, BZW1, C8orf4, COX6C, DSP, DUSP1, EEF1A1, EEF1D,
EGR1, EIF2S2, FOS, FOSB, FTH1, GOLGB1, HES1, IRX2, JUN, JUNB, MALAT1,
MGEA5, MLPH, MORF4L1, NDRG1, NEAT1, NR4A2, NUPR1, RB1CC1, RNA28S5,
RPL10, RPL10A, RPL12, RPL13A, RPL18A, RPL22, RPL23A, RPL26, RPL3, RPL36,
RPL37, RPL4, RPL41, RPL6, RPS15, RPS15A, RPS18, RPS2, RPS20, RPS25, RPS27,
RPS27A, RPS28, RPS29, RPS7, RPSA, S100A8, S100A9, SCGB1D2, SLK, SNHG9,
SRRM2, TMEM14C, TRIB1, TRPS1, UBC, UQCRB and ZFAS1
Up
AARD, ACP1, ACTR2, ACTR3, ACTR6, AIMP1, ANP32B, AP2M1, APIP, APOD, APOL6,
ARF1, ATAD2, ATP5G3, B2M, C1orf43, CALM1, CCT3, CCT4, CCT5, CCT6A, CD44,
CD46, CD74, CHCHD2, COA6, COPS2, COX7A2, COX7C, COX8A, CP, EBNA1BP2,
EIF2AK2, EIF5, EPRS, EZH2, FKBP3, GBP1, GTF3A, H2AFZ, HDGF, HIST1H1D,
HIST1H4C, HLA-B, HLA-C, HNRNPA2B1, HSBP1, HSP90B1, IFI16, IFI27, IFI44L, IFI6,
IFIH1, IFIT3, IFNAR1, IL6ST, ISG15, LAMP2, LMAN1, MAPKAP1, MDH1, MED10,
MGP, MRPL13, MX1, NDUFA1, NDUFA4, NDUFB2, NUCB2, OAZ1, PAICS, PALLD,
PAPOLA, PARP1, PARP9, PDCD5, PDHX, PDIA6, PEG10, PGRMC1, PKIB, PPM1G,
PPP1CB, PSMA3, PSMB8, PSME1, PSME2, PTGES3, RAN, RARRES1, RHOA, RSRC1,
S100A6, SEC61B, SEC62, SIVA1, SLC38A1, SMARCA5, SNX6, SRP72, SSR3, SSR4,
STMN1, TAP1, TCP1, TM4SF1, TMCO1, TMEM59, TMEM97, TMPO, TRAM1, TSPO,
TXNL1, UBE2J1, UBL5, UCHL5, UQCRH, USP1, VBP1 and XAF1

TABLE 3
Tumor_RvNR

| | | | | |
|---|---|---|---|---|
| p_val | avg_logFC | pct.1 | pct.2 | p_val_adj |

CSF_full.r2.set.RvNR.tumor.post.bimod.markers.dn.1

| | | | | | |
|---|---|---|---|---|---|
| RPS28 | 3.53E−170 | −0.308 | 0.225 | 0.65 | 4.02E−166 |
| PABPC1 | 3.54E−166 | −1.141 | 0.903 | 0.989 | 4.03E−162 |
| RPS20 | 9.16E−123 | −0.422 | 0.643 | 0.844 | 1.04E−118 |
| YWHAE | 1.47E−117 | −0.983 | 0.375 | 0.804 | 1.67E−113 |
| EEF1A1 | 1.00E−106 | −0.326 | 0.638 | 0.865 | 1.14E−102 |
| SNHG5 | 5.39E−105 | −0.825 | 0.432 | 0.785 | 6.14E−101 |
| EIF1 | 2.19E−102 | −0.896 | 0.45 | 0.839 | 2.50E−98 |
| PRKDC | 1.77E−101 | −2.055 | 0.18 | 0.53 | 2.02E−97 |
| RPL5 | 7.45E−98 | −0.419 | 0.627 | 0.896 | 8.49E−94 |
| TUBB | 3.47E−95 | −1.004 | 0.251 | 0.607 | 3.95E−91 |
| SERBP1 | 3.91E−91 | −0.301 | 0.339 | 0.626 | 4.45E−87 |
| SLC25A5 | 2.53E−90 | −0.621 | 0.345 | 0.691 | 2.88E−86 |
| EIF5B | 5.06E−90 | −0.739 | 0.288 | 0.597 | 5.76E−86 |
| GAPDH | 2.90E−87 | −0.813 | 0.722 | 0.924 | 3.30E−83 |
| ZFAS1 | 6.01E−87 | −0.772 | 0.404 | 0.686 | 6.85E−83 |
| CSDE1 | 9.23E−86 | −0.637 | 0.311 | 0.598 | 1.05E−81 |
| FBL | 6.65E−83 | −0.671 | 0.168 | 0.496 | 7.58E−79 |
| SOAT1 | 3.57E−81 | −0.351 | 0.097 | 0.372 | 4.07E−77 |
| APLP2 | 7.83E−81 | −1.059 | 0.271 | 0.627 | 8.93E−77 |
| RNA28S5 | 7.25E−80 | −0.582 | 0.557 | 0.856 | 8.26E−76 |
| CCT6A | 1.55E−79 | −0.716 | 0.457 | 0.706 | 1.77E−75 |
| MRAS | 7.10E−79 | −0.520 | 0.139 | 0.426 | 8.09E−75 |
| SPINT2 | 1.32E−78 | −0.499 | 0.323 | 0.627 | 1.50E−74 |
| PRRC2C | 2.41E−76 | −0.767 | 0.438 | 0.756 | 2.75E−72 |
| PTMS | 3.60E−76 | −0.962 | 0.154 | 0.478 | 4.10E−72 |
| ARF1 | 2.80E−75 | −0.980 | 0.611 | 0.796 | 3.19E−71 |
| HSPA5 | 1.36E−74 | −1.022 | 0.463 | 0.728 | 1.55E−70 |
| DHRS7 | 1.97E−74 | −0.768 | 0.195 | 0.555 | 2.24E−70 |
| CTC-338M12.5 | 2.36E−74 | −2.145 | 0.031 | 0.243 | 2.69E−70 |
| RSL1D1 | 3.59E−74 | −0.254 | 0.337 | 0.578 | 4.09E−70 |
| EEF2 | 6.81E−74 | −0.265 | 0.515 | 0.801 | 7.76E−70 |
| RAN | 4.41E−73 | −0.374 | 0.435 | 0.707 | 5.02E−69 |
| B3GNT5 | 1.30E−72 | −0.352 | 0.092 | 0.332 | 1.48E−68 |
| SNHG6 | 4.64E−71 | −0.675 | 0.211 | 0.513 | 5.29E−67 |
| YWHAB | 9.61E−71 | −0.341 | 0.502 | 0.768 | 1.09E−66 |
| ILF3 | 3.50E−70 | −0.855 | 0.246 | 0.557 | 3.99E−66 |
| PAPOLA | 4.13E−70 | −0.632 | 0.401 | 0.696 | 4.71E−66 |
| SYNCRIP | 1.34E−69 | −0.639 | 0.393 | 0.686 | 1.53E−65 |
| IPO5 | 1.72E−69 | −0.864 | 0.262 | 0.609 | 1.97E−65 |
| PPP1R1B | 1.05E−68 | −0.486 | 0.14 | 0.417 | 1.20E−64 |
| GADD45GIP1 | 8.94E−68 | −0.623 | 0.297 | 0.619 | 1.02E−63 |
| TFDP1 | 1.64E−67 | −0.317 | 0.158 | 0.438 | 1.87E−63 |
| CLTC | 2.99E−67 | −0.741 | 0.33 | 0.653 | 3.41E−63 |
| CD9 | 3.27E−67 | −0.586 | 0.321 | 0.641 | 3.73E−63 |
| ACTN4 | 7.97E−67 | −0.491 | 0.165 | 0.464 | 9.08E−63 |
| PADI2 | 2.08E−66 | −0.288 | 0.104 | 0.307 | 2.37E−62 |
| C17orf76-AS1 | 2.17E−66 | −0.515 | 0.351 | 0.628 | 2.47E−62 |
| POLR2L | 1.16E−65 | −0.307 | 0.276 | 0.552 | 1.32E−61 |
| TM9SF3 | 2.06E−65 | −0.778 | 0.248 | 0.579 | 2.35E−61 |
| EIF5 | 8.86E−65 | −0.415 | 0.378 | 0.644 | 1.01E−60 |
| PDXK | 1.08E−64 | −0.877 | 0.126 | 0.434 | 1.23E−60 |
| SLC25A6 | 1.20E−64 | −0.287 | 0.349 | 0.633 | 1.36E−60 |
| YES1 | 6.97E−64 | −0.474 | 0.186 | 0.48 | 7.95E−60 |
| AZIN1 | 1.17E−63 | −0.819 | 0.188 | 0.501 | 1.34E−59 |
| PAK2 | 1.42E−63 | −0.613 | 0.231 | 0.55 | 1.62E−59 |
| MRPS35 | 1.73E−63 | −0.797 | 0.263 | 0.556 | 1.97E−59 |
| HNRNPM | 1.83E−63 | −0.565 | 0.305 | 0.604 | 2.09E−59 |
| DDAH2 | 2.59E−63 | −0.419 | 0.104 | 0.369 | 2.95E−59 |
| LPL | 2.64E−63 | −0.280 | 0.084 | 0.284 | 3.01E−59 |
| GLUL | 5.00E−63 | −0.261 | 0.164 | 0.409 | 5.70E−59 |
| G3BP2 | 5.19E−63 | −0.803 | 0.23 | 0.554 | 5.92E−59 |
| EIF4A2 | 1.65E−62 | −0.551 | 0.267 | 0.498 | 1.88E−58 |
| RNA18S5 | 3.93E−62 | −0.375 | 0.005 | 0.187 | 4.48E−58 |
| ECHS1 | 8.94E−62 | −0.472 | 0.154 | 0.447 | 1.02E−57 |
| NOL7 | 2.18E−61 | −0.381 | 0.309 | 0.559 | 2.48E−57 |
| PRMT2 | 2.27E−61 | −0.407 | 0.261 | 0.571 | 2.58E−57 |
| HNRNPD | 2.48E−61 | −0.492 | 0.267 | 0.563 | 2.83E−57 |
| CALR | 3.89E−61 | −0.450 | 0.522 | 0.782 | 4.44E−57 |
| NAP1L1 | 4.33E−61 | −0.340 | 0.338 | 0.591 | 4.94E−57 |
| ATP5J | 6.96E−61 | −0.380 | 0.355 | 0.619 | 7.93E−57 |
| LARS | 7.07E−61 | −0.515 | 0.235 | 0.521 | 8.06E−57 |
| TPR | 7.81E−61 | −0.584 | 0.346 | 0.647 | 8.90E−57 |
| CNBP | 1.32E−60 | −0.442 | 0.349 | 0.619 | 1.50E−56 |
| RPN2 | 1.77E−60 | −0.283 | 0.185 | 0.427 | 2.01E−56 |
| DYNC1H1 | 2.42E−60 | −0.884 | 0.195 | 0.508 | 2.76E−56 |
| CTNNA1 | 2.56E−60 | −0.553 | 0.267 | 0.585 | 2.92E−56 |
| RPL27 | 3.05E−60 | −0.400 | 0.749 | 0.911 | 3.47E−56 |
| HNRNPU | 6.00E−60 | −0.659 | 0.416 | 0.715 | 6.84E−56 |
| TRPS1 | 7.29E−60 | −0.818 | 0.277 | 0.544 | 8.31E−56 |
| STRAP | 8.08E−60 | −0.743 | 0.121 | 0.418 | 9.21E−56 |
| PSMD11 | 1.66E−59 | −0.535 | 0.125 | 0.408 | 1.89E−55 |
| SSBP1 | 1.94E−59 | −0.497 | 0.297 | 0.574 | 2.21E−55 |
| KPNA4 | 2.14E−59 | −0.441 | 0.24 | 0.53 | 2.44E−55 |
| PSMD2 | 9.07E−59 | −0.619 | 0.208 | 0.507 | 1.03E−54 |
| DDX21 | 9.36E−59 | −0.570 | 0.304 | 0.587 | 1.07E−54 |
| EIF3K | 1.44E−58 | −0.257 | 0.219 | 0.449 | 1.64E−54 |
| PAICS | 1.69E−58 | −0.608 | 0.3 | 0.578 | 1.92E−54 |
| GLTSCR2 | 2.13E−58 | −0.469 | 0.23 | 0.467 | 2.43E−54 |
| YBX3 | 2.90E−58 | −0.628 | 0.139 | 0.419 | 3.31E−54 |
| CYC1 | 4.39E−58 | −0.536 | 0.17 | 0.46 | 5.00E−54 |
| CTNNB1 | 5.65E−58 | −0.601 | 0.193 | 0.482 | 6.44E−54 |
| KDELR1 | 7.61E−58 | −0.541 | 0.167 | 0.452 | 8.68E−54 |
| ITGB8 | 7.79E−58 | −0.607 | 0.138 | 0.388 | 8.88E−54 |
| PRPF40A | 1.07E−57 | −0.494 | 0.342 | 0.589 | 1.22E−53 |
| SLC38A2 | 1.15E−57 | −0.896 | 0.184 | 0.487 | 1.31E−53 |
| HDGF | 1.30E−57 | −0.396 | 0.446 | 0.679 | 1.48E−53 |
| ABLIM1 | 1.32E−57 | −0.317 | 0.168 | 0.426 | 1.50E−53 |
| SYNE2 | 1.39E−57 | −0.788 | 0.463 | 0.702 | 1.59E−53 |
| SNRPD3 | 1.87E−57 | −0.390 | 0.255 | 0.504 | 2.13E−53 |
| PARP1 | 2.43E−57 | −0.541 | 0.256 | 0.555 | 2.77E−53 |
| NET1 | 4.27E−57 | −0.524 | 0.215 | 0.487 | 4.86E−53 |
| SON | 4.92E−57 | −0.554 | 0.313 | 0.613 | 5.61E−53 |
| CCT3 | 5.10E−57 | −0.508 | 0.447 | 0.698 | 5.81E−53 |
| ITGA6 | 6.44E−57 | −0.487 | 0.139 | 0.402 | 7.34E−53 |
| 10-Sep | 9.87E−57 | −0.301 | 0.075 | 0.255 | 1.13E−52 |
| UQCRB | 2.54E−56 | −0.496 | 0.283 | 0.479 | 2.90E−52 |
| APP | 4.52E−56 | −0.539 | 0.425 | 0.715 | 5.15E−52 |
| FUS | 4.64E−56 | −0.325 | 0.206 | 0.465 | 5.29E−52 |
| DKC1 | 5.04E−56 | −0.274 | 0.236 | 0.499 | 5.75E−52 |
| KLF6 | 8.78E−56 | −0.535 | 0.401 | 0.661 | 1.00E−51 |
| MPHOSPH6 | 9.17E−56 | −0.426 | 0.185 | 0.454 | 1.05E−51 |
| LARP4B | 1.31E−55 | −0.284 | 0.148 | 0.375 | 1.50E−51 |
| EPRS | 3.62E−55 | −0.307 | 0.391 | 0.624 | 4.13E−51 |
| SEC31A | 6.44E−55 | −0.520 | 0.19 | 0.479 | 7.34E−51 |
| VCP | 7.47E−55 | −0.566 | 0.189 | 0.447 | 8.51E−51 |
| ATP1A1 | 8.06E−55 | −0.762 | 0.243 | 0.539 | 9.19E−51 |
| ANP32B | 1.09E−54 | −0.411 | 0.371 | 0.669 | 1.25E−50 |
| VCL | 1.12E−54 | −0.646 | 0.219 | 0.51 | 1.28E−50 |
| YWHAQ | 1.24E−54 | −0.266 | 0.313 | 0.567 | 1.41E−50 |
| MAP1LC3B | 2.03E−54 | −0.463 | 0.158 | 0.423 | 2.32E−50 |
| EIF4G1 | 2.12E−54 | −0.535 | 0.232 | 0.513 | 2.41E−50 |
| SRP72 | 2.49E−54 | −0.550 | 0.352 | 0.617 | 2.84E−50 |
| MCM4 | 4.32E−54 | −1.310 | 0.114 | 0.369 | 4.92E−50 |
| CHMP4B | 5.54E−54 | −0.421 | 0.15 | 0.412 | 6.31E−50 |
| MALAT1 | 5.94E−54 | −0.793 | 0.934 | 0.922 | 6.78E−50 |
| SFT2D2 | 7.06E−54 | −0.292 | 0.223 | 0.465 | 8.04E−50 |
| CCDC47 | 7.10E−54 | −0.520 | 0.242 | 0.541 | 8.09E−50 |
| H3F3B | 8.38E−54 | −0.616 | 0.72 | 0.913 | 9.55E−50 |
| TMEM123 | 8.71E−54 | −0.553 | 0.36 | 0.633 | 9.93E−50 |
| RPS6 | 1.19E−53 | −0.400 | 0.972 | 0.996 | 1.36E−49 |
| HIST1H4C | 1.23E−53 | −1.152 | 0.636 | 0.665 | 1.40E−49 |
| DEK | 1.60E−53 | −0.518 | 0.457 | 0.761 | 1.82E−49 |
| CALM1 | 1.64E−53 | −0.517 | 0.594 | 0.822 | 1.87E−49 |
| COX8A | 1.80E−53 | −0.362 | 0.402 | 0.664 | 2.05E−49 |
| RAD23A | 1.95E−53 | −0.286 | 0.219 | 0.475 | 2.22E−49 |
| STAT3 | 3.23E−53 | −0.641 | 0.126 | 0.397 | 3.69E−49 |
| C6orf106 | 3.99E−53 | −0.537 | 0.102 | 0.351 | 4.55E−49 |
| UBE2K | 5.77E−53 | −0.449 | 0.256 | 0.529 | 6.57E−49 |
| TUBA1C | 7.90E−53 | −0.412 | 0.199 | 0.453 | 9.01E−49 |
| PTPRZ1 | 1.05E−52 | −0.483 | 0.001 | 0.147 | 1.19E−48 |
| NFIB | 1.26E−52 | −0.533 | 0.322 | 0.606 | 1.43E−48 |
| ACTN1 | 1.29E−52 | −0.547 | 0.178 | 0.437 | 1.47E−48 |
| MAP1B | 1.96E−52 | −0.384 | 0.15 | 0.335 | 2.23E−48 |
| EIF3A | 2.10E−52 | −0.588 | 0.257 | 0.542 | 2.39E−48 |
| EMP1 | 3.15E−52 | −0.494 | 0.313 | 0.504 | 3.60E−48 |
| HUWE1 | 3.87E−52 | −0.588 | 0.22 | 0.509 | 4.41E−48 |
| AHNAK | 4.21E−52 | −0.842 | 0.123 | 0.386 | 4.80E−48 |
| MPZL1 | 4.23E−52 | −0.805 | 0.309 | 0.606 | 4.82E−48 |
| PTBP3 | 9.64E−52 | −0.557 | 0.297 | 0.591 | 1.10E−47 |
| MB21D1 | 1.36E−51 | −0.319 | 0.089 | 0.283 | 1.55E−47 |
| APEX1 | 1.36E−51 | −0.259 | 0.292 | 0.525 | 1.55E−47 |
| NOP56 | 4.85E−51 | −0.845 | 0.15 | 0.387 | 5.52E−47 |
| RBM25 | 8.83E−51 | −0.596 | 0.232 | 0.455 | 1.01E−46 |
| BZW2 | 9.14E−51 | −0.400 | 0.229 | 0.484 | 1.04E−46 |
| EIF5A | 1.47E−50 | −0.507 | 0.121 | 0.369 | 1.68E−46 |
| MLEC | 1.62E−50 | −0.486 | 0.199 | 0.472 | 1.84E−46 |
| CANX | 2.02E−50 | −0.718 | 0.639 | 0.836 | 2.30E−46 |
| ASH1L | 8.15E−50 | −0.548 | 0.23 | 0.497 | 9.28E−46 |
| SRRM2 | 1.34E−49 | −0.377 | 0.201 | 0.454 | 1.53E−45 |
| RPS16 | 1.62E−49 | −0.454 | 0.779 | 0.957 | 1.84E−45 |
| PSAP | 1.84E−49 | −0.415 | 0.377 | 0.635 | 2.10E−45 |
| PSMD8 | 1.96E−49 | −0.334 | 0.247 | 0.472 | 2.24E−45 |
| LDHB | 2.06E−49 | −0.489 | 0.372 | 0.592 | 2.35E−45 |
| HES1 | 2.43E−49 | −0.253 | 0.171 | 0.41 | 2.77E−45 |
| C7orf73 | 2.95E−49 | −0.292 | 0.164 | 0.409 | 3.36E−45 |
| FDFT1 | 5.30E−49 | −0.292 | 0.187 | 0.4 | 6.04E−45 |
| PSMD14 | 8.46E−49 | −0.394 | 0.227 | 0.466 | 9.64E−45 |
| EIF3L | 9.05E−49 | −0.333 | 0.228 | 0.442 | 1.03E−44 |
| TCP1 | 1.07E−48 | −0.273 | 0.295 | 0.556 | 1.22E−44 |
| SCRN1 | 1.12E−48 | −0.299 | 0.234 | 0.482 | 1.28E−44 |
| SH3BGRL | 1.41E−48 | −0.312 | 0.195 | 0.416 | 1.60E−44 |
| TMCO3 | 1.46E−48 | −0.250 | 0.18 | 0.372 | 1.66E−44 |
| YWHAH | 1.51E−48 | −0.430 | 0.166 | 0.415 | 1.72E−44 |
| SUCLG2 | 1.53E−48 | −0.279 | 0.182 | 0.435 | 1.74E−44 |
| RARS | 1.66E−48 | −0.349 | 0.203 | 0.426 | 1.89E−44 |
| RHOA | 1.69E−48 | −0.607 | 0.506 | 0.774 | 1.92E−44 |
| RBM8A | 2.10E−48 | −0.442 | 0.157 | 0.401 | 2.39E−44 |
| SMARCC1 | 2.61E−48 | −0.667 | 0.222 | 0.465 | 2.98E−44 |
| ABRACL | 2.63E−48 | −0.289 | 0.299 | 0.562 | 3.00E−44 |
| EIF2AK1 | 2.84E−48 | −0.263 | 0.256 | 0.507 | 3.23E−44 |
| PSMD4 | 3.13E−48 | −0.301 | 0.173 | 0.408 | 3.57E−44 |
| EIF4B | 3.14E−48 | −0.390 | 0.163 | 0.389 | 3.58E−44 |
| RTN3 | 3.15E−48 | −0.371 | 0.213 | 0.474 | 3.59E−44 |
| CCDC137 | 4.95E−48 | −0.411 | 0.096 | 0.328 | 5.64E−44 |
| TROVE2 | 6.42E−48 | −0.394 | 0.344 | 0.625 | 7.32E−44 |
| UBXN4 | 8.15E−48 | −0.370 | 0.273 | 0.497 | 9.29E−44 |
| LMNA | 8.93E−48 | −0.667 | 0.375 | 0.637 | 1.02E−43 |
| CAPZB | 1.01E−47 | −0.281 | 0.247 | 0.472 | 1.15E−43 |
| BCAP31 | 1.10E−47 | −0.341 | 0.305 | 0.562 | 1.25E−43 |
| SF3B5 | 1.28E−47 | −0.351 | 0.17 | 0.391 | 1.46E−43 |
| SMARCA5 | 1.44E−47 | −0.379 | 0.336 | 0.538 | 1.64E−43 |
| PKM | 1.51E−47 | −0.306 | 0.219 | 0.448 | 1.72E−43 |
| CCT2 | 1.52E−47 | −0.529 | 0.247 | 0.461 | 1.74E−43 |
| COPA | 2.34E−47 | −0.344 | 0.209 | 0.47 | 2.67E−43 |
| PRDX6 | 2.66E−47 | −0.380 | 0.208 | 0.453 | 3.03E−43 |
| LSM14A | 3.47E−47 | −0.471 | 0.173 | 0.423 | 3.96E−43 |
| ATF3 | 4.21E−47 | −0.283 | 0.187 | 0.374 | 4.80E−43 |
| DHCR24 | 5.00E−47 | −0.533 | 0.229 | 0.493 | 5.70E−43 |
| RRBP1 | 6.01E−47 | −0.310 | 0.283 | 0.537 | 6.85E−43 |
| ANXA1 | 7.46E−47 | −0.365 | 0.451 | 0.625 | 8.50E−43 |
| VIM | 1.02E−46 | −0.453 | 0.449 | 0.646 | 1.16E−42 |
| CHD4 | 1.28E−46 | −0.684 | 0.187 | 0.447 | 1.45E−42 |
| LBR | 1.64E−46 | −0.499 | 0.307 | 0.585 | 1.87E−42 |
| SSRP1 | 2.45E−46 | −0.452 | 0.26 | 0.516 | 2.79E−42 |
| NASP | 2.95E−46 | −0.305 | 0.322 | 0.572 | 3.36E−42 |
| ATP1B1 | 4.07E−46 | −0.276 | 0.265 | 0.454 | 4.64E−42 |
| NMT1 | 9.74E−46 | −0.385 | 0.095 | 0.316 | 1.11E−41 |
| HNRNPAB | 9.80E−46 | −0.291 | 0.208 | 0.44 | 1.12E−41 |
| EPB41L2 | 1.04E−45 | −0.394 | 0.066 | 0.239 | 1.18E−41 |
| PDIA4 | 1.10E−45 | −0.366 | 0.261 | 0.495 | 1.26E−41 |
| NUCB1 | 1.13E−45 | −0.280 | 0.123 | 0.343 | 1.29E−41 |
| DNMT1 | 1.37E−45 | −0.871 | 0.166 | 0.418 | 1.56E−41 |
| SERPINE2 | 1.81E−45 | −0.436 | 0.039 | 0.219 | 2.06E−41 |
| CD164 | 2.34E−45 | −0.272 | 0.359 | 0.572 | 2.66E−41 |
| SDC2 | 2.45E−45 | −1.307 | 0.003 | 0.139 | 2.79E−41 |
| ZDHHC13 | 2.69E−45 | −0.342 | 0.123 | 0.342 | 3.07E−41 |
| TAF1D | 3.02E−45 | −1.228 | 0.129 | 0.362 | 3.44E−41 |
| MRPS15 | 3.06E−45 | −0.437 | 0.215 | 0.482 | 3.49E−41 |
| SENP6 | 4.25E−45 | −0.595 | 0.259 | 0.509 | 4.85E−41 |
| ZDHHC20 | 4.91E−45 | −0.428 | 0.122 | 0.35 | 5.59E−41 |
| CBX5 | 4.99E−45 | −0.460 | 0.203 | 0.449 | 5.69E−41 |
| CD109 | 5.02E−45 | −0.450 | 0.115 | 0.301 | 5.72E−41 |
| ILF2 | 5.12E−45 | −0.400 | 0.395 | 0.668 | 5.84E−41 |
| MRPL13 | 6.88E−45 | −0.543 | 0.387 | 0.627 | 7.84E−41 |
| SLC25A3 | 7.24E−45 | −0.383 | 0.283 | 0.509 | 8.25E−41 |
| SENP5 | 7.55E−45 | −0.495 | 0.076 | 0.278 | 8.61E−41 |
| GDI2 | 7.58E−45 | −0.681 | 0.44 | 0.708 | 8.64E−41 |
| GAR1 | 1.44E−44 | −0.336 | 0.15 | 0.382 | 1.64E−40 |
| PHACTR2 | 1.73E−44 | −0.722 | 0.043 | 0.225 | 1.97E−40 |
| PTTG1IP | 1.78E−44 | −0.658 | 0.511 | 0.762 | 2.02E−40 |
| ARHGAP5 | 1.84E−44 | −0.287 | 0.283 | 0.501 | 2.09E−40 |
| EMC4 | 2.16E−44 | −0.521 | 0.187 | 0.443 | 2.47E−40 |
| SKA2 | 2.48E−44 | −0.310 | 0.195 | 0.435 | 2.83E−40 |
| C8orf33 | 3.72E−44 | −0.370 | 0.149 | 0.392 | 4.24E−40 |
| DDX5 | 3.79E−44 | −0.479 | 0.58 | 0.78 | 4.32E−40 |
| TATDN1 | 4.50E−44 | −0.342 | 0.17 | 0.41 | 5.13E−40 |
| OSTF1 | 4.53E−44 | −0.256 | 0.082 | 0.269 | 5.17E−40 |
| FAM32A | 6.51E−44 | −0.546 | 0.138 | 0.371 | 7.42E−40 |
| PPIB | 9.38E−44 | −0.382 | 0.342 | 0.583 | 1.07E−39 |
| SPP1 | 9.95E−44 | −1.251 | 0.186 | 0.367 | 1.13E−39 |
| POLR2B | 1.00E−43 | −0.551 | 0.225 | 0.478 | 1.14E−39 |
| YY1 | 1.03E−43 | −0.372 | 0.255 | 0.492 | 1.17E−39 |
| NDUFB5 | 1.10E−43 | −0.303 | 0.241 | 0.473 | 1.25E−39 |
| SEC63 | 1.32E−43 | −0.260 | 0.246 | 0.465 | 1.51E−39 |
| EIF3B | 1.90E−43 | −0.396 | 0.107 | 0.33 | 2.16E−39 |
| BPTF | 2.05E−43 | −0.585 | 0.245 | 0.493 | 2.33E−39 |
| CHAMP1 | 2.06E−43 | −0.369 | 0.097 | 0.302 | 2.35E−39 |
| HNRNPF | 2.33E−43 | −0.336 | 0.289 | 0.537 | 2.66E−39 |
| SPTBN1 | 2.50E−43 | −0.419 | 0.211 | 0.459 | 2.85E−39 |
| MPC2 | 2.51E−43 | −0.330 | 0.24 | 0.46 | 2.86E−39 |
| ARPC5L | 3.29E−43 | −0.557 | 0.236 | 0.493 | 3.75E−39 |
| SF3B2 | 4.07E−43 | −0.491 | 0.174 | 0.423 | 4.64E−39 |
| TAF2 | 5.42E−43 | −0.470 | 0.159 | 0.4 | 6.17E−39 |
| HIST1H1C | 5.64E−43 | −0.708 | 0.323 | 0.548 | 6.42E−39 |
| BRD4 | 6.31E−43 | −0.575 | 0.105 | 0.332 | 7.20E−39 |
| TMX4 | 6.81E−43 | −0.361 | 0.121 | 0.321 | 7.76E−39 |
| TXNIP | 8.11E−43 | −0.370 | 0.297 | 0.521 | 9.24E−39 |
| ENO1 | 1.02E−42 | −0.655 | 0.713 | 0.855 | 1.17E−38 |
| NAA50 | 1.04E−42 | −0.323 | 0.185 | 0.41 | 1.18E−38 |
| ZFR | 1.15E−42 | −0.407 | 0.216 | 0.43 | 1.31E−38 |
| ARMCX3 | 1.47E−42 | −0.269 | 0.285 | 0.505 | 1.68E−38 |
| EZR | 1.80E−42 | −0.629 | 0.309 | 0.569 | 2.05E−38 |
| TCF25 | 1.85E−42 | −0.261 | 0.219 | 0.441 | 2.11E−38 |
| PRPSAP1 | 1.87E−42 | −0.434 | 0.141 | 0.369 | 2.14E−38 |
| GRB2 | 2.22E−42 | −0.401 | 0.268 | 0.516 | 2.52E−38 |
| ANP32E | 2.42E−42 | −0.342 | 0.205 | 0.43 | 2.76E−38 |
| USO1 | 2.79E−42 | −0.732 | 0.162 | 0.4 | 3.18E−38 |
| COPB2 | 3.23E−42 | −0.346 | 0.288 | 0.534 | 3.69E−38 |
| ADAR | 3.30E−42 | −0.259 | 0.346 | 0.555 | 3.76E−38 |
| FKBP3 | 3.35E−42 | −0.272 | 0.35 | 0.551 | 3.82E−38 |
| LRPPRC | 4.22E−42 | −0.431 | 0.255 | 0.501 | 4.81E−38 |
| C12orf57 | 5.92E−42 | −0.398 | 0.2 | 0.426 | 6.74E−38 |
| VPS29 | 7.03E−42 | −0.345 | 0.204 | 0.419 | 8.02E−38 |
| PAK1IP1 | 7.05E−42 | −0.280 | 0.234 | 0.451 | 8.04E−38 |
| MYH9 | 7.10E−42 | −0.344 | 0.278 | 0.496 | 8.09E−38 |
| CHI3L1 | 7.37E−42 | −0.313 | 0.066 | 0.232 | 8.40E−38 |
| CLIC4 | 8.02E−42 | −0.460 | 0.172 | 0.414 | 9.15E−38 |
| UAP1 | 9.12E−42 | −0.798 | 0.154 | 0.397 | 1.04E−37 |
| ACTR3 | 9.79E−42 | −0.256 | 0.386 | 0.607 | 1.12E−37 |
| ACAT2 | 1.08E−41 | −0.389 | 0.146 | 0.362 | 1.23E−37 |
| ERP44 | 1.27E−41 | −0.534 | 0.092 | 0.308 | 1.45E−37 |
| SF3B1 | 1.47E−41 | −0.275 | 0.397 | 0.581 | 1.67E−37 |
| STARD7 | 1.63E−41 | −0.465 | 0.19 | 0.428 | 1.86E−37 |
| SCP2 | 1.92E−41 | −0.256 | 0.217 | 0.435 | 2.19E−37 |
| CCNI | 2.02E−41 | −0.329 | 0.492 | 0.724 | 2.30E−37 |
| FSTL1 | 2.31E−41 | −0.399 | 0.111 | 0.28 | 2.64E−37 |
| HSPA9 | 2.36E−41 | −0.257 | 0.27 | 0.474 | 2.69E−37 |
| TRAM1 | 2.73E−41 | −0.518 | 0.378 | 0.644 | 3.11E−37 |
| UBR5 | 3.32E−41 | −0.568 | 0.137 | 0.34 | 3.79E−37 |
| PTP4A1 | 3.66E−41 | −0.417 | 0.248 | 0.484 | 4.17E−37 |
| DNM1L | 5.45E−41 | −0.525 | 0.154 | 0.392 | 6.21E−37 |
| TKT | 5.57E−41 | −0.250 | 0.351 | 0.53 | 6.34E−37 |
| SWAP70 | 6.78E−41 | −0.323 | 0.137 | 0.335 | 7.73E−37 |
| ID2 | 9.93E−41 | −0.606 | 0.21 | 0.414 | 1.13E−36 |
| MAGOHB | 1.03E−40 | −0.434 | 0.128 | 0.345 | 1.18E−36 |
| BSG | 1.43E−40 | −0.306 | 0.144 | 0.337 | 1.63E−36 |
| NF1 | 1.72E−40 | −0.453 | 0.06 | 0.229 | 1.96E−36 |
| DDX39A | 2.80E−40 | −0.458 | 0.113 | 0.327 | 3.20E−36 |
| TAF15 | 2.99E−40 | −0.279 | 0.088 | 0.274 | 3.41E−36 |
| SLC38A1 | 3.04E−40 | −0.659 | 0.509 | 0.758 | 3.47E−36 |
| THBS1 | 3.07E−40 | −1.684 | 0.109 | 0.22 | 3.50E−36 |
| COBL | 3.37E−40 | −0.416 | 0.093 | 0.284 | 3.84E−36 |
| CALU | 3.58E−40 | −0.399 | 0.293 | 0.54 | 4.08E−36 |
| DDX46 | 7.89E−40 | −0.424 | 0.182 | 0.393 | 9.00E−36 |
| TUBGCP3 | 8.68E−40 | −0.294 | 0.121 | 0.301 | 9.90E−36 |
| SUCLG1 | 1.07E−39 | −0.412 | 0.367 | 0.564 | 1.22E−35 |
| RNF7 | 1.21E−39 | −0.253 | 0.277 | 0.451 | 1.38E−35 |
| TRA2B | 1.34E−39 | −0.324 | 0.236 | 0.459 | 1.53E−35 |
| PRPF6 | 1.39E−39 | −0.553 | 0.115 | 0.344 | 1.58E−35 |
| TMEM70 | 1.39E−39 | −0.571 | 0.176 | 0.379 | 1.59E−35 |
| CMPK1 | 1.46E−39 | −0.267 | 0.323 | 0.548 | 1.66E−35 |
| H2AFY | 1.46E−39 | −0.318 | 0.297 | 0.539 | 1.67E−35 |
| BAZ1B | 1.61E−39 | −0.387 | 0.249 | 0.458 | 1.84E−35 |
| C6orf48 | 1.77E−39 | −1.164 | 0.141 | 0.344 | 2.02E−35 |
| ARPC5 | 2.62E−39 | −0.697 | 0.777 | 0.819 | 2.99E−35 |
| SAV1 | 2.89E−39 | −0.359 | 0.096 | 0.296 | 3.29E−35 |
| PHLDA1 | 3.10E−39 | −0.424 | 0.064 | 0.227 | 3.53E−35 |
| KCTD3 | 3.33E−39 | −0.262 | 0.107 | 0.299 | 3.80E−35 |
| SPTLC2 | 3.69E−39 | −0.281 | 0.144 | 0.357 | 4.20E−35 |
| VGLL4 | 4.04E−39 | −0.282 | 0.116 | 0.306 | 4.61E−35 |
| KIF5B | 4.66E−39 | −0.297 | 0.396 | 0.613 | 5.31E−35 |
| MYOF | 5.67E−39 | −0.354 | 0.101 | 0.3 | 6.46E−35 |
| HNRNPUL1 | 5.78E−39 | −0.499 | 0.159 | 0.368 | 6.58E−35 |
| SH3GLB1 | 8.08E−39 | −0.521 | 0.184 | 0.385 | 9.20E−35 |
| UTP11L | 8.59E−39 | −0.258 | 0.188 | 0.394 | 9.79E−35 |
| PLK2 | 1.02E−38 | −0.423 | 0.155 | 0.359 | 1.16E−34 |
| CNDP2 | 1.11E−38 | −0.319 | 0.133 | 0.343 | 1.26E−34 |
| ARPC2 | 1.11E−38 | −0.312 | 0.469 | 0.689 | 1.27E−34 |
| CALM3 | 1.33E−38 | −0.340 | 0.175 | 0.382 | 1.51E−34 |
| MTDH | 1.42E−38 | −0.803 | 0.223 | 0.444 | 1.62E−34 |
| LGALS3 | 1.96E−38 | −0.629 | 0.45 | 0.643 | 2.24E−34 |
| OS9 | 2.00E−38 | −0.267 | 0.139 | 0.337 | 2.28E−34 |
| 15-Sep | 2.27E−38 | −0.510 | 0.179 | 0.387 | 2.58E−34 |
| CARS2 | 2.38E−38 | −0.390 | 0.088 | 0.283 | 2.72E−34 |
| PPA1 | 2.71E−38 | −0.327 | 0.415 | 0.63 | 3.09E−34 |
| DHX9 | 2.73E−38 | −0.378 | 0.328 | 0.571 | 3.11E−34 |
| PSME3 | 2.88E−38 | −0.580 | 0.059 | 0.252 | 3.28E−34 |
| TTC3 | 3.76E−38 | −0.297 | 0.215 | 0.371 | 4.28E−34 |
| ACTL6A | 3.84E−38 | −0.531 | 0.259 | 0.483 | 4.38E−34 |
| G3BP1 | 4.10E−38 | −0.419 | 0.212 | 0.439 | 4.68E−34 |
| CDC73 | 4.43E−38 | −0.284 | 0.219 | 0.438 | 5.05E−34 |
| PGRMC1 | 4.77E−38 | −0.491 | 0.358 | 0.584 | 5.44E−34 |
| YWHAG | 5.23E−38 | −0.297 | 0.201 | 0.419 | 5.96E−34 |
| RCAN1 | 5.54E−38 | −0.274 | 0.134 | 0.295 | 6.32E−34 |
| DDX42 | 5.75E−38 | −0.509 | 0.111 | 0.309 | 6.55E−34 |
| CREG1 | 5.96E−38 | −0.480 | 0.186 | 0.41 | 6.79E−34 |
| RAB10 | 6.65E−38 | −0.342 | 0.327 | 0.531 | 7.58E−34 |
| SNX3 | 7.72E−38 | −0.256 | 0.237 | 0.45 | 8.80E−34 |
| KIAA0368 | 8.13E−38 | −0.487 | 0.155 | 0.372 | 9.26E−34 |
| TIAL1 | 9.94E−38 | −0.275 | 0.167 | 0.377 | 1.13E−33 |
| PSMC5 | 9.95E−38 | −0.384 | 0.17 | 0.387 | 1.13E−33 |
| LAMP2 | 1.21E−37 | −0.548 | 0.41 | 0.649 | 1.38E−33 |
| LARP1 | 1.31E−37 | −0.265 | 0.13 | 0.312 | 1.50E−33 |
| UBB | 1.37E−37 | −0.333 | 0.609 | 0.744 | 1.57E−33 |
| CNOT1 | 1.50E−37 | −0.269 | 0.132 | 0.335 | 1.70E−33 |
| NRD1 | 1.52E−37 | −0.308 | 0.232 | 0.44 | 1.74E−33 |
| GHITM | 1.59E−37 | −0.277 | 0.302 | 0.527 | 1.81E−33 |
| CTC-425F1.4 | 2.54E−37 | −0.272 | 0.129 | 0.303 | 2.89E−33 |
| PSMC4 | 3.00E−37 | −0.538 | 0.118 | 0.321 | 3.42E−33 |
| C6orf62 | 3.03E−37 | −0.361 | 0.139 | 0.345 | 3.46E−33 |
| SHROOM3 | 3.03E−37 | −0.463 | 0.083 | 0.264 | 3.46E−33 |
| CDC16 | 3.24E−37 | −0.531 | 0.196 | 0.419 | 3.69E−33 |
| PAIP2 | 4.01E−37 | −0.362 | 0.215 | 0.437 | 4.57E−33 |
| TGFB2 | 4.23E−37 | −0.343 | 0 | 0.102 | 4.83E−33 |
| TPX2 | 4.25E−37 | −0.826 | 0.117 | 0.308 | 4.85E−33 |
| RIF1 | 4.66E−37 | −0.758 | 0.21 | 0.422 | 5.32E−33 |
| LCP1 | 5.69E−37 | −0.328 | 0.238 | 0.417 | 6.49E−33 |
| USP39 | 5.93E−37 | −0.261 | 0.135 | 0.332 | 6.76E−33 |
| CKB | 5.97E−37 | −0.296 | 0.051 | 0.212 | 6.80E−33 |
| GATAD2A | 6.03E−37 | −0.365 | 0.087 | 0.269 | 6.88E−33 |
| HM13 | 6.21E−37 | −0.619 | 0.106 | 0.319 | 7.08E−33 |
| GTF2A2 | 7.58E−37 | −0.251 | 0.206 | 0.407 | 8.64E−33 |
| OCIAD1 | 8.53E−37 | −0.313 | 0.275 | 0.501 | 9.73E−33 |
| ATF6 | 9.13E−37 | −0.264 | 0.24 | 0.452 | 1.04E−32 |
| STK3 | 1.18E−36 | −1.031 | 0.084 | 0.284 | 1.35E−32 |
| CTSB | 1.27E−36 | −0.341 | 0.202 | 0.407 | 1.45E−32 |
| PUS7L | 1.29E−36 | −0.412 | 0.183 | 0.397 | 1.47E−32 |
| RBBP7 | 1.49E−36 | −0.334 | 0.209 | 0.416 | 1.70E−32 |
| MAP4 | 1.55E−36 | −0.414 | 0.194 | 0.42 | 1.77E−32 |
| RAB6A | 1.56E−36 | −0.549 | 0.099 | 0.304 | 1.77E−32 |
| XPO1 | 2.01E−36 | −0.345 | 0.236 | 0.454 | 2.30E−32 |
| ITGB1BP1 | 2.26E−36 | −0.274 | 0.128 | 0.298 | 2.58E−32 |
| IPO9 | 2.33E−36 | −0.330 | 0.119 | 0.298 | 2.65E−32 |
| TBL1XR1 | 2.43E−36 | −0.351 | 0.336 | 0.557 | 2.77E−32 |
| C1orf35 | 2.82E−36 | −0.259 | 0.113 | 0.298 | 3.22E−32 |
| SPTAN1 | 3.42E−36 | −0.316 | 0.1 | 0.274 | 3.90E−32 |
| TARDBP | 3.51E−36 | −0.556 | 0.206 | 0.428 | 4.00E−32 |
| ETV3 | 3.59E−36 | −0.321 | 0.078 | 0.256 | 4.10E−32 |
| CCDC88A | 4.43E−36 | −0.277 | 0.084 | 0.225 | 5.04E−32 |
| PSMB6 | 4.44E−36 | −0.314 | 0.195 | 0.354 | 5.06E−32 |
| DNAJC3 | 4.62E−36 | −0.341 | 0.268 | 0.498 | 5.26E−32 |
| NOLC1 | 5.66E−36 | −0.475 | 0.159 | 0.374 | 6.45E−32 |
| MARCKS | 7.83E−36 | −0.295 | 0.186 | 0.389 | 8.92E−32 |
| CADM1 | 7.90E−36 | −0.489 | 0.026 | 0.161 | 9.01E−32 |
| PRRC2B | 8.65E−36 | −0.349 | 0.151 | 0.334 | 9.86E−32 |
| PSMD3 | 9.19E−36 | −0.304 | 0.073 | 0.242 | 1.05E−31 |
| SH3YL1 | 1.03E−35 | −0.328 | 0.181 | 0.376 | 1.17E−31 |
| GOLGA2 | 1.06E−35 | −0.352 | 0.101 | 0.295 | 1.21E−31 |
| PDCD10 | 1.09E−35 | −0.329 | 0.467 | 0.689 | 1.24E−31 |
| WBP11 | 1.11E−35 | −0.683 | 0.155 | 0.375 | 1.27E−31 |
| TMEM33 | 1.22E−35 | −0.461 | 0.135 | 0.353 | 1.40E−31 |
| PSMD1 | 1.42E−35 | −0.443 | 0.199 | 0.418 | 1.62E−31 |
| GFM1 | 1.60E−35 | −0.301 | 0.135 | 0.332 | 1.82E−31 |
| COPB1 | 1.83E−35 | −0.368 | 0.246 | 0.472 | 2.09E−31 |
| MOB1A | 1.94E−35 | −0.468 | 0.225 | 0.433 | 2.21E−31 |
| SYAP1 | 1.95E−35 | −0.261 | 0.26 | 0.467 | 2.22E−31 |
| DERL1 | 2.25E−35 | −0.318 | 0.105 | 0.296 | 2.57E−31 |
| GSTO1 | 2.28E−35 | −0.367 | 0.127 | 0.306 | 2.60E−31 |
| RRP15 | 2.34E−35 | −0.318 | 0.216 | 0.425 | 2.67E−31 |
| NUDCD1 | 2.69E−35 | −0.522 | 0.244 | 0.47 | 3.07E−31 |
| MIA3 | 2.82E−35 | −0.374 | 0.231 | 0.44 | 3.21E−31 |
| CCDC90B | 3.02E−35 | −0.411 | 0.166 | 0.377 | 3.44E−31 |
| SLC50A1 | 3.25E−35 | −0.344 | 0.114 | 0.308 | 3.71E−31 |
| C1orf27 | 3.38E−35 | −0.319 | 0.195 | 0.393 | 3.85E−31 |
| ZNF146 | 3.42E−35 | −0.372 | 0.247 | 0.429 | 3.90E−31 |
| SOX4 | 3.60E−35 | −0.364 | 0.404 | 0.578 | 4.11E−31 |
| SRSF3 | 3.88E−35 | −0.270 | 0.542 | 0.723 | 4.43E−31 |
| 11-Sep | 3.94E−35 | −0.995 | 0.121 | 0.325 | 4.49E−31 |
| TUBA1A | 4.32E−35 | −0.337 | 0.194 | 0.369 | 4.92E−31 |
| PNO1 | 4.56E−35 | −0.380 | 0.178 | 0.391 | 5.19E−31 |
| SLC3A2 | 4.79E−35 | −0.283 | 0.128 | 0.312 | 5.47E−31 |
| TNPO1 | 5.51E−35 | −0.303 | 0.117 | 0.296 | 6.28E−31 |
| WHSC1 | 5.72E−35 | −0.621 | 0.069 | 0.253 | 6.52E−31 |
| AFG3L2 | 8.00E−35 | −0.267 | 0.128 | 0.32 | 9.11E−31 |
| SPAG9 | 8.23E−35 | −0.471 | 0.165 | 0.374 | 9.38E−31 |
| ROCK1 | 8.36E−35 | −0.291 | 0.223 | 0.416 | 9.53E−31 |
| REST | 8.61E−35 | −0.485 | 0.131 | 0.319 | 9.81E−31 |
| KRT7 | 1.01E−34 | −0.563 | 0.565 | 0.761 | 1.15E−30 |
| BRD2 | 1.04E−34 | −0.288 | 0.215 | 0.412 | 1.19E−30 |
| ITM2B | 1.04E−34 | −0.636 | 0.4 | 0.612 | 1.19E−30 |
| SETD2 | 1.10E−34 | −0.531 | 0.13 | 0.331 | 1.25E−30 |
| ARL3 | 1.21E−34 | −0.458 | 0.15 | 0.353 | 1.38E−30 |
| NCOA2 | 1.27E−34 | −0.404 | 0.124 | 0.322 | 1.44E−30 |
| ACLY | 1.30E−34 | −0.529 | 0.087 | 0.284 | 1.48E−30 |
| NIPA2 | 1.45E−34 | −0.498 | 0.135 | 0.343 | 1.65E−30 |
| CCNC | 1.51E−34 | −0.462 | 0.196 | 0.412 | 1.73E−30 |
| ZC3H13 | 1.73E−34 | −0.299 | 0.13 | 0.328 | 1.97E−30 |
| PANK3 | 1.76E−34 | −0.291 | 0.186 | 0.379 | 2.01E−30 |
| ERRFI1 | 2.03E−34 | −0.517 | 0.128 | 0.335 | 2.31E−30 |
| UBAP2L | 2.15E−34 | −0.385 | 0.122 | 0.315 | 2.45E−30 |
| EHF | 2.49E−34 | −0.273 | 0.238 | 0.444 | 2.84E−30 |
| SLC39A7 | 2.52E−34 | −0.260 | 0.1 | 0.282 | 2.87E−30 |
| USP1 | 2.57E−34 | −0.287 | 0.406 | 0.605 | 2.93E−30 |
| SLMO2 | 2.88E−34 | −0.307 | 0.264 | 0.457 | 3.28E−30 |
| TOR1AIP1 | 3.28E−34 | −0.300 | 0.155 | 0.341 | 3.74E−30 |
| TUBGCP2 | 3.55E−34 | −0.536 | 0.14 | 0.346 | 4.05E−30 |
| TMEM30A | 4.43E−34 | −0.427 | 0.239 | 0.477 | 5.05E−30 |
| KIAA1598 | 4.46E−34 | −0.432 | 0.102 | 0.281 | 5.08E−30 |
| EIF2S1 | 4.47E−34 | −0.353 | 0.214 | 0.419 | 5.10E−30 |
| CAST | 4.54E−34 | −0.324 | 0.187 | 0.386 | 5.18E−30 |
| TPD52 | 4.63E−34 | −0.532 | 0.158 | 0.377 | 5.28E−30 |
| DNAH14 | 4.77E−34 | −0.398 | 0.152 | 0.356 | 5.44E−30 |
| GGPS1 | 4.80E−34 | −0.348 | 0.178 | 0.378 | 5.47E−30 |
| CAP1 | 5.08E−34 | −0.414 | 0.445 | 0.657 | 5.79E−30 |
| CNIH1 | 5.61E−34 | −0.461 | 0.21 | 0.418 | 6.40E−30 |
| NDUFA10 | 9.26E−34 | −0.260 | 0.195 | 0.395 | 1.06E−29 |
| MSMO1 | 9.34E−34 | −0.347 | 0.231 | 0.434 | 1.06E−29 |
| VPS4B | 9.85E−34 | −0.412 | 0.153 | 0.353 | 1.12E−29 |
| CKAP5 | 1.05E−33 | −0.502 | 0.124 | 0.324 | 1.20E−29 |
| PNRC1 | 1.08E−33 | −0.275 | 0.137 | 0.329 | 1.23E−29 |
| TEX10 | 1.09E−33 | −0.310 | 0.088 | 0.266 | 1.24E−29 |
| SCNM1 | 1.14E−33 | −0.331 | 0.106 | 0.302 | 1.30E−29 |
| SQLE | 1.17E−33 | −0.617 | 0.416 | 0.622 | 1.34E−29 |
| WNK1 | 1.21E−33 | −0.434 | 0.121 | 0.315 | 1.38E−29 |
| TPP2 | 1.35E−33 | −0.265 | 0.146 | 0.338 | 1.53E−29 |
| FBXO9 | 1.59E−33 | −0.336 | 0.188 | 0.4 | 1.81E−29 |
| ZMPSTE24 | 1.79E−33 | −0.446 | 0.223 | 0.441 | 2.04E−29 |
| VPS26A | 1.97E−33 | −0.349 | 0.166 | 0.369 | 2.24E−29 |
| HEATR1 | 2.31E−33 | −0.503 | 0.149 | 0.364 | 2.63E−29 |
| ANXA5 | 2.57E−33 | −0.345 | 0.353 | 0.588 | 2.93E−29 |
| LSM2 | 2.76E−33 | −0.409 | 0.1 | 0.294 | 3.14E−29 |
| TFRC | 2.99E−33 | −0.498 | 0.219 | 0.423 | 3.41E−29 |
| CUL4A | 3.13E−33 | −0.294 | 0.104 | 0.272 | 3.57E−29 |
| PARL | 3.46E−33 | −0.378 | 0.077 | 0.252 | 3.94E−29 |
| PIK3R1 | 3.84E−33 | −0.261 | 0.219 | 0.38 | 4.38E−29 |
| SUCO | 3.86E−33 | −0.668 | 0.206 | 0.427 | 4.40E−29 |
| DNAJB1 | 4.80E−33 | −0.575 | 0.184 | 0.36 | 5.47E−29 |
| TMPO | 5.20E−33 | −0.294 | 0.268 | 0.456 | 5.92E−29 |
| USP36 | 5.60E−33 | −0.300 | 0.053 | 0.202 | 6.38E−29 |
| HSPH1 | 5.84E−33 | −0.292 | 0.321 | 0.519 | 6.66E−29 |
| RAB3GAP2 | 6.84E−33 | −0.353 | 0.17 | 0.378 | 7.80E−29 |
| TES | 7.64E−33 | −0.268 | 0.236 | 0.432 | 8.71E−29 |
| NFE2L2 | 8.67E−33 | −0.376 | 0.135 | 0.333 | 9.88E−29 |
| DDX24 | 8.75E−33 | −0.333 | 0.175 | 0.369 | 9.97E−29 |
| UPP1 | 8.92E−33 | −0.343 | 0.031 | 0.161 | 1.02E−28 |
| FIP1L1 | 9.78E−33 | −0.448 | 0.121 | 0.318 | 1.12E−28 |
| MRPS34 | 1.07E−32 | −0.357 | 0.301 | 0.473 | 1.22E−28 |
| SZRD1 | 1.13E−32 | −0.297 | 0.117 | 0.292 | 1.28E−28 |
| PLIN2 | 1.33E−32 | −0.590 | 0.145 | 0.339 | 1.52E−28 |
| ARL6IP1 | 1.47E−32 | −0.462 | 0.307 | 0.52 | 1.68E−28 |
| TMOD3 | 1.47E−32 | −0.348 | 0.173 | 0.379 | 1.68E−28 |
| HSP90B1 | 1.96E−32 | −0.469 | 0.693 | 0.806 | 2.23E−28 |
| SACS | 2.11E−32 | −0.524 | 0.03 | 0.157 | 2.41E−28 |
| MIB1 | 2.30E−32 | −0.518 | 0.141 | 0.332 | 2.62E−28 |
| BMS1 | 3.05E−32 | −0.493 | 0.133 | 0.333 | 3.47E−28 |
| POR | 3.08E−32 | −0.370 | 0.064 | 0.233 | 3.52E−28 |
| GMNN | 3.45E−32 | −0.476 | 0.122 | 0.302 | 3.94E−28 |
| USP14 | 4.12E−32 | −0.588 | 0.183 | 0.403 | 4.70E−28 |
| XRN2 | 4.51E−32 | −0.416 | 0.203 | 0.407 | 5.14E−28 |
| ABI1 | 4.90E−32 | −0.489 | 0.252 | 0.448 | 5.59E−28 |
| TMEM106C | 5.19E−32 | −0.270 | 0.236 | 0.411 | 5.91E−28 |
| PPP1CC | 5.57E−32 | −0.350 | 0.176 | 0.365 | 6.34E−28 |
| MAGED2 | 6.08E−32 | −0.287 | 0.162 | 0.332 | 6.93E−28 |
| UBAC1 | 6.20E−32 | −0.290 | 0.086 | 0.233 | 7.07E−28 |
| SPEN | 6.36E−32 | −0.291 | 0.171 | 0.359 | 7.25E−28 |
| TET1 | 6.74E−32 | −0.467 | 0.031 | 0.164 | 7.68E−28 |
| KIF1B | 7.49E−32 | −0.404 | 0.084 | 0.25 | 8.53E−28 |
| DPYSL2 | 8.98E−32 | −0.541 | 0.059 | 0.228 | 1.02E−27 |
| FAM60A | 1.00E−31 | −0.520 | 0.125 | 0.315 | 1.14E−27 |
| AGFG1 | 1.02E−31 | −0.275 | 0.105 | 0.251 | 1.17E−27 |
| SETX | 1.10E−31 | −0.429 | 0.19 | 0.375 | 1.25E−27 |
| TSG101 | 1.11E−31 | −0.354 | 0.142 | 0.328 | 1.27E−27 |
| METAP2 | 1.21E−31 | −0.312 | 0.202 | 0.385 | 1.38E−27 |
| NCOA4 | 1.46E−31 | −0.325 | 0.145 | 0.331 | 1.67E−27 |
| HIST2H2AC | 1.59E−31 | −0.467 | 0.087 | 0.265 | 1.81E−27 |
| FAM208B | 1.64E−31 | −0.454 | 0.208 | 0.401 | 1.87E−27 |
| C19orf43 | 1.83E−31 | −0.387 | 0.157 | 0.347 | 2.08E−27 |
| ATF7IP | 1.94E−31 | −0.532 | 0.079 | 0.256 | 2.21E−27 |
| ENOPH1 | 2.02E−31 | −0.275 | 0.112 | 0.293 | 2.30E−27 |
| KMT2A | 2.15E−31 | −0.881 | 0.058 | 0.222 | 2.45E−27 |
| PBX1 | 2.19E−31 | −0.343 | 0.166 | 0.346 | 2.49E−27 |
| PIK3C2A | 2.19E−31 | −0.441 | 0.109 | 0.293 | 2.50E−27 |
| UBE2V2 | 2.62E−31 | −0.669 | 0.15 | 0.357 | 2.99E−27 |
| ABCF1 | 2.89E−31 | −0.705 | 0.115 | 0.309 | 3.30E−27 |
| ACIN1 | 2.99E−31 | −0.292 | 0.163 | 0.347 | 3.41E−27 |
| TGFBR1 | 3.03E−31 | −0.658 | 0.035 | 0.176 | 3.45E−27 |
| MCMBP | 3.47E−31 | −0.567 | 0.096 | 0.279 | 3.95E−27 |
| SFXN1 | 5.32E−31 | −0.483 | 0.1 | 0.283 | 6.07E−27 |
| FBXO32 | 5.67E−31 | −1.015 | 0.028 | 0.169 | 6.46E−27 |
| CWC15 | 5.71E−31 | −0.546 | 0.196 | 0.397 | 6.51E−27 |
| ZNF106 | 7.49E−31 | −0.323 | 0.131 | 0.307 | 8.54E−27 |
| CHMP2B | 8.23E−31 | −0.323 | 0.294 | 0.482 | 9.38E−27 |
| RANBP2 | 8.80E−31 | −0.297 | 0.148 | 0.321 | 1.00E−26 |
| KIDINS220 | 8.93E−31 | −0.482 | 0.117 | 0.293 | 1.02E−26 |
| E2F3 | 9.14E−31 | −0.324 | 0.071 | 0.218 | 1.04E−26 |
| MOB1B | 9.60E−31 | −0.769 | 0.055 | 0.215 | 1.09E−26 |
| IER2 | 1.06E−30 | −0.528 | 0.091 | 0.267 | 1.20E−26 |
| PIP5K1A | 1.06E−30 | −0.427 | 0.068 | 0.234 | 1.21E−26 |
| NSUN2 | 1.11E−30 | −0.283 | 0.113 | 0.288 | 1.26E−26 |
| KCTD12 | 1.22E−30 | −0.294 | 0.025 | 0.138 | 1.39E−26 |
| USP9X | 1.28E−30 | −0.374 | 0.113 | 0.284 | 1.46E−26 |
| EIF3M | 1.34E−30 | −0.280 | 0.273 | 0.453 | 1.53E−26 |
| IARS | 1.39E−30 | −0.482 | 0.138 | 0.334 | 1.58E−26 |
| UCHL5 | 1.44E−30 | −0.342 | 0.319 | 0.52 | 1.64E−26 |
| LOX | 1.44E−30 | −0.479 | 0.023 | 0.132 | 1.65E−26 |
| PMP22 | 1.45E−30 | −0.404 | 0.186 | 0.309 | 1.65E−26 |
| DPY19L4 | 1.52E−30 | −0.492 | 0.055 | 0.217 | 1.73E−26 |
| S100A10 | 1.70E−30 | −0.337 | 0.532 | 0.672 | 1.93E−26 |
| POGK | 2.09E−30 | −0.374 | 0.129 | 0.304 | 2.38E−26 |
| FAT1 | 2.13E−30 | −0.278 | 0.056 | 0.17 | 2.43E−26 |
| TLN1 | 2.28E−30 | −0.436 | 0.22 | 0.402 | 2.60E−26 |
| ATAD2 | 3.08E−30 | −0.338 | 0.199 | 0.358 | 3.52E−26 |
| PICALM | 3.88E−30 | −0.453 | 0.11 | 0.291 | 4.42E−26 |
| CYB5R3 | 3.94E−30 | −0.359 | 0.098 | 0.25 | 4.49E−26 |
| XPOT | 4.09E−30 | −0.362 | 0.11 | 0.288 | 4.66E−26 |
| PYURF | 4.95E−30 | −0.296 | 0.314 | 0.508 | 5.64E−26 |
| CLINT1 | 5.65E−30 | −0.293 | 0.199 | 0.377 | 6.44E−26 |
| SPG21 | 5.70E−30 | −0.355 | 0.104 | 0.279 | 6.49E−26 |
| KMT2C | 6.02E−30 | −0.488 | 0.162 | 0.353 | 6.86E−26 |
| ANKLE2 | 7.38E−30 | −0.401 | 0.061 | 0.224 | 8.41E−26 |
| ARCN1 | 7.69E−30 | −0.737 | 0.084 | 0.263 | 8.77E−26 |
| SNRNP200 | 7.74E−30 | −0.501 | 0.122 | 0.316 | 8.83E−26 |
| BTG1 | 7.84E−30 | −0.568 | 0.109 | 0.298 | 8.94E−26 |
| IRF6 | 9.07E−30 | −0.348 | 0.128 | 0.297 | 1.03E−25 |
| ZNF638 | 9.13E−30 | −0.361 | 0.236 | 0.434 | 1.04E−25 |
| NSMCE2 | 1.09E−29 | −0.256 | 0.168 | 0.337 | 1.25E−25 |
| RDX | 1.13E−29 | −0.736 | 0.108 | 0.282 | 1.28E−25 |
| NOTCH2 | 1.18E−29 | −0.393 | 0.066 | 0.217 | 1.35E−25 |
| ETF1 | 1.29E−29 | −0.353 | 0.235 | 0.437 | 1.47E−25 |
| DNAJC10 | 1.36E−29 | −0.398 | 0.227 | 0.431 | 1.54E−25 |
| BROX | 1.46E−29 | −0.427 | 0.322 | 0.545 | 1.66E−25 |
| TMEM167A | 1.51E−29 | −0.278 | 0.173 | 0.336 | 1.72E−25 |
| MKI67 | 1.72E−29 | −0.487 | 0.276 | 0.413 | 1.96E−25 |
| WBSCR22 | 1.93E−29 | −0.349 | 0.131 | 0.313 | 2.20E−25 |
| IARS2 | 2.03E−29 | −0.370 | 0.367 | 0.578 | 2.31E−25 |
| GTF2F1 | 2.55E−29 | −0.383 | 0.115 | 0.298 | 2.91E−25 |
| UBE2J2 | 2.65E−29 | −0.264 | 0.074 | 0.224 | 3.02E−25 |
| UBQLN1 | 2.85E−29 | −0.283 | 0.202 | 0.396 | 3.25E−25 |
| RALGPS2 | 2.90E−29 | −0.285 | 0.078 | 0.229 | 3.30E−25 |
| PABPC4 | 3.51E−29 | −0.301 | 0.124 | 0.297 | 4.00E−25 |
| SMC4 | 3.55E−29 | −0.501 | 0.371 | 0.543 | 4.05E−25 |
| ARHGEF12 | 3.57E−29 | −0.896 | 0.072 | 0.24 | 4.06E−25 |
| EXOC3 | 3.87E−29 | −0.547 | 0.104 | 0.271 | 4.41E−25 |
| MED10 | 5.40E−29 | −0.381 | 0.236 | 0.415 | 6.16E−25 |
| PCID2 | 5.52E−29 | −0.534 | 0.205 | 0.404 | 6.29E−25 |
| IGFBP3 | 6.14E−29 | −1.589 | 0.042 | 0.157 | 6.99E−25 |
| GARS | 6.25E−29 | −0.312 | 0.229 | 0.408 | 7.13E−25 |
| TXNRD1 | 6.39E−29 | −0.317 | 0.167 | 0.338 | 7.28E−25 |
| PSMD12 | 6.55E−29 | −0.276 | 0.264 | 0.461 | 7.47E−25 |
| TRIP12 | 6.85E−29 | −0.279 | 0.202 | 0.374 | 7.81E−25 |
| RPLP1 | 7.10E−29 | −0.469 | 0.945 | 0.947 | 8.09E−25 |
| NUP54 | 7.13E−29 | −0.547 | 0.087 | 0.261 | 8.13E−25 |
| ZNF511 | 7.24E−29 | −0.368 | 0.105 | 0.269 | 8.25E−25 |
| RNF168 | 7.75E−29 | −0.307 | 0.197 | 0.381 | 8.83E−25 |
| SAR1A | 8.78E−29 | −0.366 | 0.193 | 0.371 | 1.00E−24 |
| RAB18 | 1.00E−28 | −0.316 | 0.125 | 0.285 | 1.14E−24 |
| HNRNPL | 1.05E−28 | −0.260 | 0.11 | 0.275 | 1.20E−24 |
| PDE4DIP | 1.06E−28 | −0.611 | 0.051 | 0.202 | 1.21E−24 |
| CMTM6 | 1.14E−28 | −0.275 | 0.199 | 0.378 | 1.30E−24 |
| NAT10 | 1.16E−28 | −0.298 | 0.086 | 0.248 | 1.33E−24 |
| CPSF3 | 1.35E−28 | −0.292 | 0.076 | 0.232 | 1.53E−24 |
| ZNF124 | 1.39E−28 | −0.402 | 0.084 | 0.249 | 1.58E−24 |
| JMJD1C | 1.60E−28 | −0.450 | 0.175 | 0.346 | 1.83E−24 |
| GRSF1 | 1.60E−28 | −0.361 | 0.162 | 0.351 | 1.83E−24 |
| CD46 | 1.66E−28 | −0.355 | 0.483 | 0.636 | 1.89E−24 |
| CAPZA1 | 1.69E−28 | −0.294 | 0.184 | 0.367 | 1.92E−24 |
| TSR2 | 1.85E−28 | −0.323 | 0.126 | 0.297 | 2.11E−24 |
| GLO1 | 1.90E−28 | −0.293 | 0.376 | 0.563 | 2.16E−24 |
| NUFIP2 | 1.95E−28 | −0.290 | 0.186 | 0.356 | 2.22E−24 |
| OPA1 | 2.40E−28 | −0.285 | 0.204 | 0.392 | 2.74E−24 |
| GPR107 | 2.50E−28 | −0.335 | 0.047 | 0.184 | 2.85E−24 |
| SUPT5H | 2.57E−28 | −0.369 | 0.105 | 0.28 | 2.93E−24 |
| AP2B1 | 2.77E−28 | −0.488 | 0.098 | 0.262 | 3.16E−24 |
| ATXN10 | 2.82E−28 | −0.275 | 0.186 | 0.36 | 3.22E−24 |
| NOP16 | 2.83E−28 | −0.319 | 0.154 | 0.336 | 3.22E−24 |
| PAFAH1B1 | 3.03E−28 | −0.480 | 0.134 | 0.322 | 3.45E−24 |
| SLC36A4 | 3.28E−28 | −0.627 | 0.02 | 0.139 | 3.74E−24 |
| MAGED1 | 3.39E−28 | −0.331 | 0.092 | 0.259 | 3.86E−24 |
| SMEK2 | 3.40E−28 | −0.293 | 0.268 | 0.465 | 3.88E−24 |
| PRMT1 | 3.75E−28 | −0.289 | 0.091 | 0.246 | 4.28E−24 |
| BDP1 | 4.53E−28 | −0.334 | 0.204 | 0.377 | 5.16E−24 |
| SMEK1 | 5.17E−28 | −0.764 | 0.117 | 0.297 | 5.89E−24 |
| C12orf23 | 5.34E−28 | −0.357 | 0.109 | 0.276 | 6.08E−24 |
| TMBIM6 | 5.39E−28 | −0.478 | 0.559 | 0.725 | 6.14E−24 |
| TIMMDC1 | 5.84E−28 | −0.429 | 0.162 | 0.34 | 6.66E−24 |
| LPP | 6.19E−28 | −0.309 | 0.211 | 0.381 | 7.06E−24 |
| AHR | 7.33E−28 | −0.725 | 0.113 | 0.283 | 8.35E−24 |
| ZNF37A | 8.02E−28 | −0.278 | 0.092 | 0.234 | 9.14E−24 |
| SCARB2 | 8.08E−28 | −0.360 | 0.207 | 0.382 | 9.21E−24 |
| DIAPH1 | 8.29E−28 | −0.302 | 0.112 | 0.273 | 9.44E−24 |
| MPC1 | 9.23E−28 | −0.430 | 0.177 | 0.359 | 1.05E−23 |
| TRA2A | 9.51E−28 | −0.449 | 0.131 | 0.309 | 1.08E−23 |
| TFG | 1.11E−27 | −0.414 | 0.176 | 0.363 | 1.26E−23 |
| MRPL21 | 1.18E−27 | −0.265 | 0.117 | 0.279 | 1.34E−23 |
| YIF1A | 1.28E−27 | −1.200 | 0.115 | 0.242 | 1.46E−23 |
| AVL9 | 1.44E−27 | −0.431 | 0.09 | 0.247 | 1.64E−23 |
| ANXA7 | 1.68E−27 | −0.314 | 0.141 | 0.313 | 1.92E−23 |
| UBE2A | 1.79E−27 | −0.279 | 0.085 | 0.235 | 2.05E−23 |
| CHML | 1.80E−27 | −0.883 | 0.124 | 0.299 | 2.05E−23 |
| IRS2 | 1.93E−27 | −0.528 | 0.104 | 0.253 | 2.20E−23 |
| ZNF33A | 2.42E−27 | −0.303 | 0.122 | 0.282 | 2.76E−23 |
| SLK | 2.50E−27 | −0.376 | 0.136 | 0.305 | 2.85E−23 |
| KHSRP | 2.58E−27 | −0.282 | 0.101 | 0.258 | 2.94E−23 |
| USP34 | 2.99E−27 | −0.276 | 0.196 | 0.345 | 3.41E−23 |
| GGNBP2 | 3.22E−27 | −0.254 | 0.103 | 0.257 | 3.67E−23 |
| DICER1 | 3.34E−27 | −0.712 | 0.132 | 0.319 | 3.81E−23 |
| TM9SF2 | 3.50E−27 | −0.612 | 0.592 | 0.748 | 3.98E−23 |
| URI1 | 4.56E−27 | −0.256 | 0.193 | 0.349 | 5.20E−23 |
| ANAPC5 | 4.69E−27 | −0.527 | 0.196 | 0.381 | 5.35E−23 |
| CFL2 | 4.72E−27 | −0.269 | 0.064 | 0.201 | 5.38E−23 |
| CYFIP1 | 5.07E−27 | −0.426 | 0.118 | 0.294 | 5.78E−23 |
| MACF1 | 5.77E−27 | −0.337 | 0.227 | 0.392 | 6.57E−23 |
| TMED4 | 5.99E−27 | −0.381 | 0.196 | 0.388 | 6.83E−23 |
| LIN7C | 6.03E−27 | −0.355 | 0.125 | 0.288 | 6.88E−23 |
| DSC2 | 6.33E−27 | −0.449 | 0.361 | 0.538 | 7.21E−23 |
| CAPZA2 | 7.27E−27 | −0.251 | 0.204 | 0.365 | 8.28E−23 |
| NUDT21 | 9.45E−27 | −0.270 | 0.198 | 0.368 | 1.08E−22 |
| ADK | 9.84E−27 | −0.401 | 0.166 | 0.344 | 1.12E−22 |
| SGK1 | 1.09E−26 | −0.635 | 0.026 | 0.139 | 1.24E−22 |
| ATRX | 1.14E−26 | −0.569 | 0.205 | 0.383 | 1.30E−22 |
| KCNQ1OT1 | 1.22E−26 | −0.969 | 0.045 | 0.16 | 1.39E−22 |
| METTL9 | 1.42E−26 | −0.338 | 0.115 | 0.277 | 1.61E−22 |
| AZGP1 | 1.43E−26 | −0.395 | 0.715 | 0.636 | 1.63E−22 |
| MRFAP1 | 1.47E−26 | −0.389 | 0.111 | 0.277 | 1.68E−22 |
| IER3 | 1.53E−26 | −1.081 | 0.076 | 0.208 | 1.74E−22 |
| CORO1C | 1.66E−26 | −0.382 | 0.071 | 0.213 | 1.89E−22 |
| MED13 | 1.72E−26 | −0.500 | 0.141 | 0.316 | 1.97E−22 |
| SORT1 | 1.94E−26 | −0.475 | 0.071 | 0.229 | 2.21E−22 |
| IPO7 | 1.96E−26 | −0.382 | 0.205 | 0.378 | 2.24E−22 |
| SPTLC1 | 2.03E−26 | −0.274 | 0.081 | 0.226 | 2.31E−22 |
| CEBPG | 2.22E−26 | −0.332 | 0.194 | 0.375 | 2.53E−22 |
| PCNT | 2.24E−26 | −0.325 | 0.085 | 0.237 | 2.56E−22 |
| ASCC3 | 2.47E−26 | −0.398 | 0.145 | 0.314 | 2.82E−22 |
| STMN1 | 2.56E−26 | −0.307 | 0.469 | 0.663 | 2.92E−22 |
| NUP153 | 2.68E−26 | −0.332 | 0.067 | 0.201 | 3.05E−22 |
| TEX30 | 3.11E−26 | −0.332 | 0.109 | 0.26 | 3.55E−22 |
| MX2 | 3.13E−26 | −0.276 | 0.077 | 0.198 | 3.57E−22 |
| UGGT2 | 3.17E−26 | −0.253 | 0.117 | 0.266 | 3.61E−22 |
| SUV420H1 | 3.25E−26 | −0.468 | 0.101 | 0.261 | 3.70E−22 |
| GPATCH8 | 3.75E−26 | −0.463 | 0.053 | 0.189 | 4.27E−22 |
| ANLN | 4.28E−26 | −0.292 | 0.211 | 0.356 | 4.88E−22 |
| PDIA6 | 4.31E−26 | −0.499 | 0.494 | 0.656 | 4.92E−22 |
| PAPSS1 | 4.49E−26 | −0.286 | 0.241 | 0.42 | 5.11E−22 |
| VTI1B | 4.60E−26 | −0.453 | 0.083 | 0.243 | 5.25E−22 |
| RPP38 | 5.41E−26 | −0.496 | 0.149 | 0.329 | 6.17E−22 |
| MCM6 | 5.43E−26 | −0.453 | 0.058 | 0.196 | 6.18E−22 |
| CCDC6 | 5.47E−26 | −0.331 | 0.15 | 0.318 | 6.24E−22 |
| ELOVL5 | 5.66E−26 | −0.259 | 0.188 | 0.356 | 6.45E−22 |
| CPD | 6.07E−26 | −0.425 | 0.046 | 0.177 | 6.92E−22 |
| PTK2 | 6.34E−26 | −0.329 | 0.133 | 0.301 | 7.23E−22 |
| AFAP1 | 6.36E−26 | −0.502 | 0.018 | 0.126 | 7.25E−22 |
| FUBP1 | 6.50E−26 | −0.401 | 0.164 | 0.345 | 7.41E−22 |
| ATP6AP2 | 6.88E−26 | −0.298 | 0.148 | 0.309 | 7.84E−22 |
| SEPHS2 | 6.94E−26 | −0.253 | 0.121 | 0.278 | 7.91E−22 |
| NEDD9 | 8.35E−26 | −0.294 | 0.1 | 0.234 | 9.51E−22 |
| MAGT1 | 9.69E−26 | −0.367 | 0.126 | 0.29 | 1.10E−21 |
| SMNDC1 | 9.92E−26 | −0.367 | 0.1 | 0.251 | 1.13E−21 |
| NAA15 | 1.06E−25 | −0.371 | 0.184 | 0.364 | 1.21E−21 |
| NOL11 | 1.20E−25 | −0.302 | 0.142 | 0.314 | 1.36E−21 |
| HNRNPA2B1 | 1.33E−25 | −0.347 | 0.789 | 0.924 | 1.52E−21 |
| GFPT1 | 1.39E−25 | −0.308 | 0.16 | 0.328 | 1.59E−21 |
| ATL3 | 1.41E−25 | −0.447 | 0.112 | 0.274 | 1.61E−21 |
| LYPLAL1 | 1.48E−25 | −0.279 | 0.121 | 0.256 | 1.68E−21 |
| ANKRD17 | 1.54E−25 | −0.556 | 0.133 | 0.301 | 1.75E−21 |
| RUFY3 | 1.78E−25 | −0.294 | 0.084 | 0.228 | 2.03E−21 |
| HMG20B | 2.17E−25 | −0.331 | 0.204 | 0.377 | 2.47E−21 |
| SESN3 | 2.19E−25 | −0.918 | 0.013 | 0.112 | 2.49E−21 |
| NEAT1 | 2.34E−25 | −0.489 | 0.112 | 0.241 | 2.67E−21 |
| RNF115 | 2.64E−25 | −0.436 | 0.129 | 0.294 | 3.01E−21 |
| HIPK3 | 2.64E−25 | −0.352 | 0.109 | 0.25 | 3.01E−21 |
| RAB8A | 2.70E−25 | −0.266 | 0.15 | 0.31 | 3.08E−21 |
| TRMT2B | 2.98E−25 | −0.290 | 0.049 | 0.167 | 3.40E−21 |
| PIGC | 3.12E−25 | −0.329 | 0.067 | 0.213 | 3.55E−21 |
| IFNGR1 | 3.14E−25 | −0.307 | 0.215 | 0.388 | 3.58E−21 |
| DLG1 | 3.31E−25 | −0.478 | 0.108 | 0.27 | 3.78E−21 |
| CDK13 | 4.25E−25 | −0.369 | 0.067 | 0.207 | 4.84E−21 |
| MRPS5 | 4.35E−25 | −0.312 | 0.177 | 0.34 | 4.96E−21 |
| CPVL | 4.46E−25 | −0.343 | 0.018 | 0.115 | 5.08E−21 |
| TMEM165 | 4.57E−25 | −0.535 | 0.432 | 0.592 | 5.21E−21 |
| IDI1 | 4.85E−25 | −0.330 | 0.395 | 0.57 | 5.53E−21 |
| NCALD | 5.36E−25 | −0.342 | 0.022 | 0.132 | 6.11E−21 |
| SRSF11 | 5.57E−25 | −0.368 | 0.17 | 0.327 | 6.35E−21 |
| NCOR1 | 5.97E−25 | −0.753 | 0.077 | 0.23 | 6.80E−21 |
| MPLKIP | 6.26E−25 | −0.313 | 0.131 | 0.302 | 7.13E−21 |
| ABI2 | 6.69E−25 | −0.684 | 0.088 | 0.251 | 7.62E−21 |
| DHX36 | 8.10E−25 | −0.447 | 0.183 | 0.359 | 9.23E−21 |
| OSBPL2 | 8.35E−25 | −0.413 | 0.054 | 0.194 | 9.52E−21 |
| CCNK | 8.87E−25 | −0.560 | 0.092 | 0.25 | 1.01E−20 |
| TIA1 | 1.03E−24 | −0.281 | 0.178 | 0.329 | 1.17E−20 |
| RNF8 | 2.08E−24 | −0.287 | 0.067 | 0.201 | 2.37E−20 |
| SPTSSA | 2.19E−24 | −0.293 | 0.16 | 0.323 | 2.50E−20 |
| PPM1G | 2.44E−24 | −0.369 | 0.399 | 0.587 | 2.78E−20 |
| BIRC2 | 2.50E−24 | −0.832 | 0.101 | 0.26 | 2.84E−20 |
| CPNE1 | 2.57E−24 | −0.297 | 0.079 | 0.227 | 2.93E−20 |
| RAP1GDS1 | 2.91E−24 | −0.251 | 0.064 | 0.188 | 3.31E−20 |
| UBAC2 | 2.98E−24 | −0.263 | 0.084 | 0.225 | 3.39E−20 |
| MLX | 3.17E−24 | −0.279 | 0.063 | 0.193 | 3.61E−20 |
| GXYLT1 | 3.59E−24 | −0.365 | 0.103 | 0.236 | 4.09E−20 |
| TMX1 | 3.68E−24 | −0.303 | 0.186 | 0.335 | 4.20E−20 |
| EIF1B | 3.70E−24 | −0.381 | 0.092 | 0.247 | 4.22E−20 |
| VAMP8 | 3.88E−24 | −0.462 | 0.43 | 0.603 | 4.42E−20 |
| DHX8 | 4.21E−24 | −0.605 | 0.043 | 0.175 | 4.80E−20 |
| PGM3 | 4.79E−24 | −0.430 | 0.108 | 0.254 | 5.46E−20 |
| SYPL1 | 5.24E−24 | −0.333 | 0.169 | 0.323 | 5.97E−20 |
| SPCS3 | 5.71E−24 | −0.475 | 0.17 | 0.335 | 6.51E−20 |
| SMARCA4 | 5.87E−24 | −0.620 | 0.075 | 0.222 | 6.70E−20 |
| TMEM87A | 5.89E−24 | −0.307 | 0.183 | 0.345 | 6.71E−20 |
| ACACA | 5.90E−24 | −0.412 | 0.027 | 0.136 | 6.72E−20 |
| EPS15 | 8.00E−24 | −0.370 | 0.079 | 0.226 | 9.12E−20 |
| KRAS | 8.44E−24 | −0.441 | 0.121 | 0.283 | 9.62E−20 |
| MFGE8 | 8.50E−24 | −0.385 | 0.74 | 0.682 | 9.69E−20 |
| SARS | 8.54E−24 | −0.310 | 0.15 | 0.298 | 9.73E−20 |
| MAP4K4 | 1.05E−23 | −0.365 | 0.04 | 0.133 | 1.19E−19 |
| CPSF2 | 1.26E−23 | −0.490 | 0.135 | 0.303 | 1.44E−19 |
| PURB | 1.27E−23 | −0.336 | 0.118 | 0.273 | 1.45E−19 |
| ATP2A2 | 1.28E−23 | −0.468 | 0.116 | 0.272 | 1.46E−19 |
| NIPBL | 1.31E−23 | −0.286 | 0.213 | 0.34 | 1.50E−19 |
| CD63 | 1.37E−23 | −0.301 | 0.488 | 0.675 | 1.56E−19 |
| REEP5 | 1.50E−23 | −0.471 | 0.195 | 0.327 | 1.71E−19 |
| SMURF2 | 1.50E−23 | −0.730 | 0.049 | 0.177 | 1.71E−19 |
| GJC1 | 1.75E−23 | −0.255 | 0.022 | 0.102 | 2.00E−19 |
| MBTPS1 | 1.90E−23 | −0.374 | 0.146 | 0.316 | 2.17E−19 |
| GTF3A | 2.25E−23 | −0.643 | 0.434 | 0.569 | 2.56E−19 |
| ELAVL1 | 2.33E−23 | −0.263 | 0.051 | 0.178 | 2.66E−19 |
| CD55 | 2.48E−23 | −0.312 | 0.358 | 0.509 | 2.83E−19 |
| RUVBL1 | 3.81E−23 | −0.273 | 0.128 | 0.28 | 4.34E−19 |
| HSPA1A | 3.90E−23 | −0.340 | 0.127 | 0.243 | 4.44E−19 |
| ICMT | 4.08E−23 | −0.309 | 0.075 | 0.211 | 4.65E−19 |
| ACSL4 | 5.44E−23 | −0.647 | 0.05 | 0.181 | 6.20E−19 |
| UBXN7 | 5.64E−23 | −0.543 | 0.06 | 0.197 | 6.43E−19 |
| ABL2 | 5.82E−23 | −1.047 | 0.053 | 0.179 | 6.63E−19 |
| SEL1L | 6.68E−23 | −0.431 | 0.087 | 0.233 | 7.62E−19 |
| FAM171B | 7.30E−23 | −0.250 | 0.031 | 0.104 | 8.32E−19 |
| DYNLL2 | 7.42E−23 | −0.275 | 0.137 | 0.282 | 8.45E−19 |
| PUM2 | 7.99E−23 | −0.260 | 0.125 | 0.261 | 9.10E−19 |
| TOP2A | 8.98E−23 | −0.775 | 0.16 | 0.297 | 1.02E−18 |
| METTL5 | 9.14E−23 | −0.272 | 0.134 | 0.276 | 1.04E−18 |
| SBNO1 | 9.36E−23 | −0.444 | 0.118 | 0.269 | 1.07E−18 |
| SENP2 | 9.81E−23 | −0.304 | 0.075 | 0.209 | 1.12E−18 |
| DNAJC2 | 1.01E−22 | −0.355 | 0.162 | 0.313 | 1.15E−18 |
| ERGIC2 | 1.02E−22 | −0.384 | 0.145 | 0.302 | 1.16E−18 |
| SMARCC2 | 1.04E−22 | −0.302 | 0.07 | 0.2 | 1.19E−18 |
| DENND1B | 1.05E−22 | −0.275 | 0.195 | 0.331 | 1.20E−18 |
| TNRC6B | 1.21E−22 | −0.310 | 0.1 | 0.246 | 1.38E−18 |
| SLC35F2 | 1.38E−22 | −0.891 | 0.03 | 0.103 | 1.58E−18 |
| SLC30A9 | 1.41E−22 | −0.270 | 0.167 | 0.325 | 1.60E−18 |
| NCAPD2 | 1.66E−22 | −0.502 | 0.044 | 0.165 | 1.89E−18 |
| DDB1 | 1.67E−22 | −0.409 | 0.091 | 0.24 | 1.90E−18 |
| LAMTOR1 | 1.82E−22 | −0.275 | 0.068 | 0.199 | 2.08E−18 |
| GTF2I | 1.83E−22 | −0.331 | 0.137 | 0.291 | 2.08E−18 |
| UBAP2 | 1.93E−22 | −0.633 | 0.131 | 0.285 | 2.21E−18 |
| UCHL1 | 2.06E−22 | −0.336 | 0.087 | 0.203 | 2.35E−18 |
| RB1CC1 | 2.26E−22 | −0.882 | 0.132 | 0.285 | 2.57E−18 |
| ACAP2 | 2.80E−22 | −0.262 | 0.145 | 0.304 | 3.19E−18 |
| PPP4R1 | 3.12E−22 | −0.423 | 0.064 | 0.187 | 3.55E−18 |
| GNPNAT1 | 3.18E−22 | −0.259 | 0.112 | 0.254 | 3.62E−18 |
| ADIPOR2 | 3.20E−22 | −0.419 | 0.095 | 0.249 | 3.64E−18 |
| UBA6 | 4.53E−22 | −0.404 | 0.101 | 0.254 | 5.16E−18 |
| LRP6 | 4.79E−22 | −0.497 | 0.045 | 0.171 | 5.46E−18 |
| RSF1 | 5.00E−22 | −0.590 | 0.163 | 0.302 | 5.70E−18 |
| EIF2B3 | 5.32E−22 | −0.311 | 0.161 | 0.312 | 6.06E−18 |
| TFAM | 5.36E−22 | −0.682 | 0.138 | 0.299 | 6.11E−18 |
| HSD17B12 | 5.55E−22 | −0.413 | 0.475 | 0.67 | 6.33E−18 |
| LYPLA1 | 5.80E−22 | −0.379 | 0.157 | 0.305 | 6.62E−18 |
| PPP2R5E | 7.44E−22 | −0.316 | 0.138 | 0.277 | 8.48E−18 |
| GLS | 7.49E−22 | −0.275 | 0.111 | 0.247 | 8.54E−18 |
| RCHY1 | 7.56E−22 | −0.348 | 0.072 | 0.207 | 8.62E−18 |
| SRSF4 | 1.19E−21 | −0.324 | 0.166 | 0.329 | 1.36E−17 |
| ZNF664 | 1.24E−21 | −0.286 | 0.081 | 0.207 | 1.41E−17 |
| FAIM | 1.28E−21 | −0.262 | 0.125 | 0.245 | 1.46E−17 |
| CNTLN | 1.36E−21 | −0.326 | 0.034 | 0.141 | 1.55E−17 |
| CHD8 | 1.47E−21 | −0.422 | 0.158 | 0.301 | 1.67E−17 |
| BIRC5 | 1.62E−21 | −0.428 | 0.186 | 0.326 | 1.85E−17 |
| PRDM2 | 1.70E−21 | −0.440 | 0.076 | 0.211 | 1.93E−17 |
| PPFIBP1 | 1.81E−21 | −0.787 | 0.077 | 0.213 | 2.06E−17 |
| PPP1R15A | 1.87E−21 | −0.501 | 0.104 | 0.26 | 2.13E−17 |
| FRYL | 2.00E−21 | −0.565 | 0.1 | 0.241 | 2.28E−17 |
| NFKBIZ | 2.53E−21 | −0.392 | 0.053 | 0.176 | 2.88E−17 |
| PRNP | 2.61E−21 | −0.370 | 0.05 | 0.166 | 2.97E−17 |
| PMAIP1 | 2.63E−21 | −0.300 | 0.132 | 0.236 | 3.00E−17 |
| AHCY | 2.76E−21 | −0.269 | 0.119 | 0.24 | 3.14E−17 |
| FAM91A1 | 2.97E−21 | −0.261 | 0.107 | 0.237 | 3.38E−17 |
| UBTF | 3.09E−21 | −0.543 | 0.108 | 0.252 | 3.53E−17 |
| ADAM10 | 3.12E−21 | −0.278 | 0.11 | 0.234 | 3.55E−17 |
| CUL5 | 3.29E−21 | −0.861 | 0.115 | 0.245 | 3.75E−17 |
| TIPRL | 3.42E−21 | −0.286 | 0.285 | 0.447 | 3.90E−17 |
| PCM1 | 3.42E−21 | −0.701 | 0.17 | 0.295 | 3.90E−17 |
| 7-Mar | 3.88E−21 | −0.547 | 0.146 | 0.315 | 4.43E−17 |
| WDR3 | 3.89E−21 | −0.348 | 0.126 | 0.265 | 4.43E−17 |
| HIST1H1D | 4.25E−21 | −0.382 | 0.162 | 0.273 | 4.84E−17 |
| DDX54 | 4.30E−21 | −0.287 | 0.05 | 0.168 | 4.90E−17 |
| ARHGEF7 | 4.46E−21 | −0.264 | 0.076 | 0.197 | 5.09E−17 |
| PTPLB | 5.31E−21 | −0.475 | 0.15 | 0.308 | 6.06E−17 |
| USP24 | 6.23E−21 | −0.340 | 0.038 | 0.143 | 7.10E−17 |
| SCAND1 | 7.15E−21 | −0.283 | 0.125 | 0.264 | 8.15E−17 |
| MDN1 | 7.51E−21 | −0.579 | 0.067 | 0.203 | 8.56E−17 |
| SDAD1 | 7.56E−21 | −0.293 | 0.119 | 0.251 | 8.62E−17 |
| KIF11 | 8.15E−21 | −0.338 | 0.066 | 0.159 | 9.29E−17 |
| DDX6 | 8.54E−21 | −0.594 | 0.118 | 0.275 | 9.73E−17 |
| GLG1 | 8.70E−21 | −0.252 | 0.117 | 0.258 | 9.92E−17 |
| SMC2 | 9.28E−21 | −0.301 | 0.108 | 0.24 | 1.06E−16 |
| POLD3 | 1.16E−20 | −0.380 | 0.052 | 0.173 | 1.32E−16 |
| PSIP1 | 1.25E−20 | −0.287 | 0.1 | 0.227 | 1.42E−16 |
| DYRK1A | 1.41E−20 | −0.334 | 0.048 | 0.15 | 1.60E−16 |
| MAPK1IP1L | 1.54E−20 | −0.269 | 0.106 | 0.249 | 1.76E−16 |
| ACOX1 | 1.67E−20 | −0.346 | 0.069 | 0.201 | 1.90E−16 |
| NCBP1 | 1.71E−20 | −0.411 | 0.077 | 0.212 | 1.95E−16 |
| STX17 | 2.00E−20 | −0.270 | 0.08 | 0.208 | 2.28E−16 |
| KANSL2 | 2.00E−20 | −0.375 | 0.026 | 0.128 | 2.28E−16 |
| NSRP1 | 2.12E−20 | −0.278 | 0.121 | 0.257 | 2.42E−16 |
| MARK3 | 2.43E−20 | −0.648 | 0.103 | 0.249 | 2.77E−16 |
| CCNG2 | 2.62E−20 | −0.876 | 0.09 | 0.215 | 2.98E−16 |
| FAM126A | 2.64E−20 | −0.365 | 0.059 | 0.167 | 3.01E−16 |
| PRPF8 | 3.12E−20 | −0.464 | 0.053 | 0.175 | 3.56E−16 |
| NEDD4L | 3.48E−20 | −0.524 | 0.063 | 0.191 | 3.96E−16 |
| CPQ | 3.69E−20 | −0.408 | 0.023 | 0.119 | 4.20E−16 |
| TDRKH | 3.85E−20 | −0.312 | 0.035 | 0.141 | 4.39E−16 |
| KDM2A | 4.35E−20 | −0.369 | 0.074 | 0.202 | 4.95E−16 |
| HIST1H1B | 4.40E−20 | −1.067 | 0.092 | 0.195 | 5.02E−16 |
| RIOK3 | 4.47E−20 | −0.272 | 0.211 | 0.37 | 5.09E−16 |
| DHX40 | 4.57E−20 | −0.539 | 0.13 | 0.277 | 5.21E−16 |
| ETNK1 | 4.99E−20 | −0.258 | 0.108 | 0.24 | 5.69E−16 |
| WDR33 | 5.16E−20 | −0.288 | 0.117 | 0.254 | 5.88E−16 |
| MDM2 | 7.02E−20 | −0.521 | 0.109 | 0.249 | 8.00E−16 |
| PRRC1 | 7.27E−20 | −0.267 | 0.078 | 0.204 | 8.29E−16 |
| ASF1A | 7.35E−20 | −0.281 | 0.128 | 0.267 | 8.38E−16 |
| ARHGAP10 | 7.88E−20 | −0.287 | 0.037 | 0.122 | 8.98E−16 |
| GART | 8.22E−20 | −0.389 | 0.117 | 0.261 | 9.37E−16 |
| TACC3 | 8.37E−20 | −0.314 | 0.038 | 0.132 | 9.54E−16 |
| ZCCHC11 | 9.14E−20 | −0.698 | 0.092 | 0.226 | 1.04E−15 |
| TSC22D2 | 9.41E−20 | −0.363 | 0.072 | 0.193 | 1.07E−15 |
| NLN | 1.28E−19 | −0.472 | 0.089 | 0.228 | 1.46E−15 |
| HIST1H1E | 1.33E−19 | −0.575 | 0.15 | 0.297 | 1.52E−15 |
| TSR1 | 1.43E−19 | −0.837 | 0.077 | 0.2 | 1.63E−15 |
| PAXBP1 | 1.44E−19 | −0.267 | 0.059 | 0.15 | 1.65E−15 |
| CENPF | 1.45E−19 | −0.558 | 0.421 | 0.465 | 1.65E−15 |
| RC3H1 | 1.45E−19 | −0.343 | 0.07 | 0.199 | 1.66E−15 |
| ATXN2L | 1.46E−19 | −0.272 | 0.043 | 0.154 | 1.66E−15 |
| STAG2 | 1.54E−19 | −0.319 | 0.176 | 0.302 | 1.76E−15 |
| CHEK1 | 1.56E−19 | −0.670 | 0.046 | 0.159 | 1.78E−15 |
| GANAB | 1.62E−19 | −0.406 | 0.063 | 0.186 | 1.85E−15 |
| SLC4A7 | 1.66E−19 | −0.456 | 0.087 | 0.217 | 1.89E−15 |
| TNPO3 | 1.96E−19 | −0.405 | 0.035 | 0.142 | 2.23E−15 |
| BAG5 | 2.19E−19 | −0.434 | 0.089 | 0.228 | 2.50E−15 |
| IFRD1 | 2.38E−19 | −0.633 | 0.089 | 0.222 | 2.71E−15 |
| AP1G1 | 2.54E−19 | −0.370 | 0.097 | 0.238 | 2.89E−15 |
| FAM98A | 2.55E−19 | −0.297 | 0.076 | 0.201 | 2.91E−15 |
| STX6 | 2.55E−19 | −0.252 | 0.078 | 0.189 | 2.91E−15 |
| HYOU1 | 2.63E−19 | −0.611 | 0.068 | 0.194 | 3.00E−15 |
| RAB3IP | 2.91E−19 | −0.312 | 0.061 | 0.178 | 3.32E−15 |
| BNIP3 | 3.00E−19 | −0.501 | 0.166 | 0.282 | 3.42E−15 |
| ENDOD1 | 3.22E−19 | −0.595 | 0.051 | 0.17 | 3.67E−15 |
| VPS13A | 3.69E−19 | −0.389 | 0.08 | 0.196 | 4.20E−15 |
| LTBR | 4.26E−19 | −0.314 | 0.088 | 0.214 | 4.86E−15 |
| PPP1CB | 4.38E−19 | −0.288 | 0.539 | 0.698 | 4.99E−15 |
| DCBLD1 | 4.45E−19 | −0.484 | 0.034 | 0.134 | 5.08E−15 |
| CDK2 | 4.66E−19 | −0.262 | 0.053 | 0.156 | 5.31E−15 |
| PPAT | 5.13E−19 | −0.434 | 0.109 | 0.256 | 5.84E−15 |
| PLEC | 5.25E−19 | −0.346 | 0.041 | 0.128 | 5.99E−15 |
| ODC1 | 5.37E−19 | −0.508 | 0.047 | 0.164 | 6.12E−15 |
| SMAP1 | 5.48E−19 | −0.304 | 0.097 | 0.226 | 6.24E−15 |
| CPNE3 | 5.54E−19 | −0.538 | 0.158 | 0.286 | 6.31E−15 |
| KLHL24 | 5.75E−19 | −0.325 | 0.056 | 0.167 | 6.55E−15 |
| MTMR12 | 6.03E−19 | −0.358 | 0.125 | 0.274 | 6.87E−15 |
| MRPL39 | 6.59E−19 | −0.385 | 0.055 | 0.175 | 7.52E−15 |
| PHF14 | 6.77E−19 | −0.500 | 0.125 | 0.264 | 7.72E−15 |
| ZNF480 | 7.05E−19 | −0.251 | 0.075 | 0.197 | 8.03E−15 |
| SFSWAP | 7.57E−19 | −0.294 | 0.081 | 0.208 | 8.62E−15 |
| MARS | 1.04E−18 | −0.290 | 0.073 | 0.195 | 1.18E−14 |
| FUBP3 | 1.05E−18 | −0.309 | 0.056 | 0.161 | 1.19E−14 |
| TIMM44 | 2.07E−18 | −0.314 | 0.119 | 0.253 | 2.36E−14 |
| TTL | 2.39E−18 | −0.438 | 0.043 | 0.147 | 2.72E−14 |
| SDCBP | 3.08E−18 | −0.256 | 0.076 | 0.179 | 3.51E−14 |
| ACTR1A | 3.15E−18 | −0.251 | 0.059 | 0.172 | 3.59E−14 |
| CNOT7 | 3.63E−18 | −0.252 | 0.129 | 0.259 | 4.14E−14 |
| GPATCH4 | 3.94E−18 | −0.378 | 0.121 | 0.257 | 4.49E−14 |
| CCNB1 | 4.19E−18 | −0.312 | 0.105 | 0.204 | 4.77E−14 |
| CUL2 | 4.37E−18 | −0.277 | 0.139 | 0.268 | 4.98E−14 |
| REV3L | 5.50E−18 | −0.315 | 0.1 | 0.204 | 6.27E−14 |
| KMT2E | 5.55E−18 | −0.307 | 0.158 | 0.283 | 6.33E−14 |
| MGA | 5.67E−18 | −0.490 | 0.042 | 0.149 | 6.46E−14 |
| SETD5 | 5.72E−18 | −0.405 | 0.113 | 0.245 | 6.52E−14 |
| VPS37B | 6.51E−18 | −0.323 | 0.039 | 0.137 | 7.42E−14 |
| SLC25A4 | 6.59E−18 | −0.413 | 0.071 | 0.195 | 7.51E−14 |
| HEBP2 | 6.84E−18 | −0.476 | 0.662 | 0.662 | 7.79E−14 |
| CMAS | 6.92E−18 | −0.280 | 0.144 | 0.285 | 7.88E−14 |
| C12orf29 | 7.77E−18 | −0.266 | 0.104 | 0.218 | 8.86E−14 |
| RFC2 | 8.43E−18 | −0.279 | 0.052 | 0.152 | 9.61E−14 |
| TCOF1 | 8.66E−18 | −0.279 | 0.072 | 0.183 | 9.87E−14 |
| BACH1 | 9.30E−18 | −0.314 | 0.084 | 0.194 | 1.06E−13 |
| FTSJ3 | 1.01E−17 | −0.389 | 0.079 | 0.202 | 1.15E−13 |
| MED1 | 1.04E−17 | −0.353 | 0.072 | 0.186 | 1.18E−13 |
| VCPIP1 | 1.08E−17 | −0.316 | 0.069 | 0.172 | 1.23E−13 |
| RIPK2 | 1.14E−17 | −0.736 | 0.064 | 0.181 | 1.30E−13 |
| ESYT2 | 1.28E−17 | −0.418 | 0.077 | 0.204 | 1.46E−13 |
| GPBP1L1 | 1.41E−17 | −0.322 | 0.139 | 0.274 | 1.61E−13 |
| ALG2 | 1.53E−17 | −0.260 | 0.103 | 0.222 | 1.75E−13 |
| PHACTR4 | 1.54E−17 | −0.297 | 0.088 | 0.21 | 1.75E−13 |
| VAPB | 1.58E−17 | −0.298 | 0.112 | 0.245 | 1.81E−13 |
| AKAP8L | 1.73E−17 | −0.299 | 0.03 | 0.121 | 1.97E−13 |
| FKTN | 1.89E−17 | −0.264 | 0.038 | 0.132 | 2.16E−13 |
| SKIV2L2 | 1.91E−17 | −0.490 | 0.1 | 0.229 | 2.18E−13 |
| DYNC1LI2 | 1.94E−17 | −0.272 | 0.154 | 0.284 | 2.21E−13 |
| ALKBH5 | 1.97E−17 | −0.280 | 0.035 | 0.13 | 2.25E−13 |
| DLGAP5 | 2.05E−17 | −0.361 | 0.068 | 0.159 | 2.33E−13 |
| CDKN1B | 2.55E−17 | −0.627 | 0.096 | 0.227 | 2.91E−13 |
| B3GALNT2 | 2.65E−17 | −0.411 | 0.042 | 0.146 | 3.02E−13 |
| SUGP2 | 2.80E−17 | −0.424 | 0.051 | 0.161 | 3.19E−13 |
| PTCD3 | 2.87E−17 | −0.287 | 0.113 | 0.234 | 3.28E−13 |
| MRPL15 | 3.20E−17 | −0.483 | 0.078 | 0.203 | 3.65E−13 |
| FLII | 3.34E−17 | −0.383 | 0.059 | 0.175 | 3.81E−13 |
| ETS1 | 3.80E−17 | −0.681 | 0.077 | 0.178 | 4.33E−13 |
| CRLF3 | 4.32E−17 | −0.353 | 0.021 | 0.106 | 4.93E−13 |
| PPFIA1 | 4.61E−17 | −0.437 | 0.072 | 0.173 | 5.25E−13 |
| FUCA2 | 4.75E−17 | −0.350 | 0.109 | 0.235 | 5.42E−13 |
| ERC1 | 5.00E−17 | −0.312 | 0.068 | 0.163 | 5.70E−13 |
| GPN3 | 5.55E−17 | −0.370 | 0.096 | 0.22 | 6.32E−13 |
| NBN | 5.60E−17 | −0.814 | 0.119 | 0.234 | 6.38E−13 |
| CDK12 | 6.12E−17 | −0.274 | 0.088 | 0.196 | 6.98E−13 |
| PSMA7 | 6.30E−17 | −0.305 | 0.624 | 0.749 | 7.18E−13 |
| TGS1 | 6.33E−17 | −0.830 | 0.084 | 0.185 | 7.22E−13 |
| BRD8 | 6.41E−17 | −0.296 | 0.055 | 0.156 | 7.31E−13 |
| MYSM1 | 6.87E−17 | −0.260 | 0.088 | 0.208 | 7.83E−13 |
| CREB3 | 7.46E−17 | −0.412 | 0.076 | 0.199 | 8.50E−13 |
| MAML2 | 7.57E−17 | −0.289 | 0.028 | 0.11 | 8.63E−13 |
| HSP90AB1 | 7.63E−17 | −0.353 | 0.891 | 0.921 | 8.70E−13 |
| NCAPG2 | 9.64E−17 | −0.315 | 0.049 | 0.148 | 1.10E−12 |
| KIF4A | 9.68E−17 | −0.287 | 0.033 | 0.118 | 1.10E−12 |
| MDC1 | 1.01E−16 | −0.330 | 0.031 | 0.119 | 1.15E−12 |
| UXS1 | 1.09E−16 | −0.259 | 0.09 | 0.213 | 1.24E−12 |
| UBR7 | 1.14E−16 | −0.422 | 0.059 | 0.16 | 1.30E−12 |
| CLCN3 | 1.31E−16 | −0.260 | 0.099 | 0.209 | 1.50E−12 |
| DIDO1 | 1.37E−16 | −0.280 | 0.089 | 0.2 | 1.56E−12 |
| MGEA5 | 1.54E−16 | −0.425 | 0.115 | 0.243 | 1.76E−12 |
| DCUN1D5 | 1.55E−16 | −0.448 | 0.093 | 0.214 | 1.77E−12 |
| BIRC6 | 1.63E−16 | −0.387 | 0.109 | 0.233 | 1.86E−12 |
| UBA2 | 1.76E−16 | −0.258 | 0.166 | 0.288 | 2.01E−12 |
| MLTK | 2.17E−16 | −0.514 | 0.075 | 0.179 | 2.47E−12 |
| GOPC | 2.55E−16 | −0.293 | 0.08 | 0.191 | 2.91E−12 |
| PTPN12 | 2.64E−16 | −0.335 | 0.105 | 0.234 | 3.01E−12 |
| STK38L | 2.66E−16 | −0.415 | 0.101 | 0.193 | 3.03E−12 |
| RPLP2 | 2.71E−16 | −0.334 | 0.852 | 0.912 | 3.09E−12 |
| DUSP16 | 2.91E−16 | −0.399 | 0.035 | 0.131 | 3.32E−12 |
| NOM1 | 3.22E−16 | −0.301 | 0.041 | 0.141 | 3.67E−12 |
| UBE2H | 3.28E−16 | −0.332 | 0.129 | 0.25 | 3.73E−12 |
| RANBP3 | 3.47E−16 | −0.328 | 0.049 | 0.144 | 3.96E−12 |
| RRM1 | 4.23E−16 | −0.289 | 0.11 | 0.229 | 4.82E−12 |
| EXOC7 | 4.34E−16 | −0.264 | 0.075 | 0.165 | 4.94E−12 |
| NOTCH2NL | 4.43E−16 | −0.262 | 0.027 | 0.115 | 5.05E−12 |
| KLHL42 | 5.80E−16 | −0.528 | 0.055 | 0.158 | 6.61E−12 |
| CEP95 | 6.17E−16 | −0.314 | 0.084 | 0.2 | 7.03E−12 |
| LARP4 | 7.43E−16 | −0.312 | 0.096 | 0.215 | 8.47E−12 |
| ERCC6L2 | 7.53E−16 | −0.391 | 0.047 | 0.145 | 8.59E−12 |
| HK1 | 8.12E−16 | −0.257 | 0.06 | 0.16 | 9.25E−12 |
| HOOK1 | 8.19E−16 | −0.252 | 0.164 | 0.289 | 9.33E−12 |
| DCP2 | 8.57E−16 | −0.255 | 0.097 | 0.199 | 9.77E−12 |
| ALDH18A1 | 8.65E−16 | −0.385 | 0.049 | 0.153 | 9.86E−12 |
| IQGAP1 | 8.96E−16 | −0.327 | 0.623 | 0.771 | 1.02E−11 |
| VTA1 | 9.87E−16 | −0.263 | 0.147 | 0.264 | 1.12E−11 |
| ARV1 | 9.87E−16 | −0.294 | 0.117 | 0.235 | 1.12E−11 |
| LUZP1 | 1.01E−15 | −0.364 | 0.071 | 0.179 | 1.16E−11 |
| RAP1A | 1.15E−15 | −0.332 | 0.111 | 0.232 | 1.31E−11 |
| CHRAC1 | 1.18E−15 | −0.300 | 0.082 | 0.199 | 1.34E−11 |
| HUS1 | 1.31E−15 | −0.253 | 0.073 | 0.175 | 1.49E−11 |
| CCNE2 | 1.35E−15 | −0.357 | 0.078 | 0.189 | 1.53E−11 |
| NUP160 | 1.56E−15 | −0.305 | 0.043 | 0.14 | 1.78E−11 |
| CNOT2 | 1.57E−15 | −0.258 | 0.11 | 0.231 | 1.78E−11 |
| PAWR | 1.67E−15 | −0.415 | 0.098 | 0.218 | 1.90E−11 |
| RASSF8 | 1.68E−15 | −0.323 | 0.03 | 0.109 | 1.92E−11 |
| TRIM24 | 1.75E−15 | −0.267 | 0.124 | 0.243 | 1.99E−11 |
| CRCP | 2.23E−15 | −0.279 | 0.072 | 0.184 | 2.54E−11 |
| ZNF121 | 2.36E−15 | −0.402 | 0.088 | 0.206 | 2.69E−11 |
| RBPJ | 2.53E−15 | −0.288 | 0.09 | 0.21 | 2.89E−11 |
| ALCAM | 2.63E−15 | −0.375 | 0.109 | 0.222 | 3.00E−11 |
| MAST2 | 2.64E−15 | −0.283 | 0.046 | 0.127 | 3.01E−11 |
| YTHDF3 | 2.83E−15 | −0.311 | 0.073 | 0.186 | 3.22E−11 |
| CITED2 | 2.86E−15 | −0.418 | 0.083 | 0.194 | 3.26E−11 |
| NUP205 | 3.92E−15 | −0.486 | 0.054 | 0.154 | 4.47E−11 |
| LRPAP1 | 4.08E−15 | −0.348 | 0.075 | 0.183 | 4.65E−11 |
| KEAP1 | 4.48E−15 | −0.411 | 0.084 | 0.201 | 5.11E−11 |
| MYBL1 | 5.18E−15 | −0.492 | 0.029 | 0.108 | 5.91E−11 |
| FAR1 | 5.63E−15 | −0.460 | 0.067 | 0.174 | 6.42E−11 |
| TNKS2 | 5.69E−15 | −0.376 | 0.048 | 0.141 | 6.48E−11 |
| PPM1A | 6.75E−15 | −0.268 | 0.085 | 0.199 | 7.69E−11 |
| PGM2L1 | 7.37E−15 | −0.668 | 0.047 | 0.139 | 8.40E−11 |
| ADAM9 | 7.43E−15 | −0.297 | 0.092 | 0.205 | 8.47E−11 |
| FBXO18 | 7.53E−15 | −0.304 | 0.029 | 0.106 | 8.58E−11 |
| TMEM245 | 7.53E−15 | −0.338 | 0.065 | 0.153 | 8.58E−11 |
| PPP2R2A | 8.54E−15 | −0.524 | 0.111 | 0.225 | 9.73E−11 |
| BLOC1S2 | 9.31E−15 | −0.277 | 0.092 | 0.198 | 1.06E−10 |
| DLAT | 9.41E−15 | −0.494 | 0.05 | 0.146 | 1.07E−10 |
| KLHL23 | 9.67E−15 | −0.386 | 0.09 | 0.203 | 1.10E−10 |
| LIG4 | 1.01E−14 | −0.287 | 0.078 | 0.183 | 1.15E−10 |
| PJA2 | 1.03E−14 | −0.290 | 0.13 | 0.256 | 1.17E−10 |
| FRMD6 | 1.11E−14 | −0.662 | 0.046 | 0.13 | 1.27E−10 |
| PPP6R3 | 1.48E−14 | −0.268 | 0.079 | 0.175 | 1.68E−10 |
| TMEM43 | 1.51E−14 | −0.360 | 0.096 | 0.215 | 1.72E−10 |
| TCEA1 | 1.51E−14 | −0.279 | 0.088 | 0.19 | 1.72E−10 |
| CCDC117 | 1.55E−14 | −0.351 | 0.11 | 0.218 | 1.77E−10 |
| CCDC91 | 1.63E−14 | −0.525 | 0.1 | 0.207 | 1.86E−10 |
| BRWD1 | 1.65E−14 | −0.508 | 0.144 | 0.265 | 1.88E−10 |
| VPS41 | 1.80E−14 | −0.291 | 0.113 | 0.213 | 2.05E−10 |
| IVD | 1.90E−14 | −0.488 | 0.084 | 0.191 | 2.17E−10 |
| STAU2 | 1.97E−14 | −0.381 | 0.087 | 0.19 | 2.24E−10 |
| CAPN2 | 2.00E−14 | −0.316 | 0.59 | 0.701 | 2.28E−10 |
| TP53BP1 | 2.01E−14 | −0.328 | 0.055 | 0.153 | 2.29E−10 |
| ARFGEF1 | 2.02E−14 | −0.266 | 0.11 | 0.218 | 2.30E−10 |
| GLIPR2 | 2.07E−14 | −0.310 | 0.042 | 0.127 | 2.35E−10 |
| SMARCB1 | 2.11E−14 | −0.332 | 0.086 | 0.196 | 2.40E−10 |
| PHF6 | 2.12E−14 | −0.253 | 0.117 | 0.219 | 2.42E−10 |
| PSMG1 | 2.18E−14 | −0.336 | 0.116 | 0.215 | 2.49E−10 |
| FGD4 | 2.28E−14 | −0.331 | 0.035 | 0.11 | 2.59E−10 |
| SGCB | 2.60E−14 | −0.407 | 0.074 | 0.177 | 2.97E−10 |
| GTF2A1 | 2.67E−14 | −0.333 | 0.059 | 0.16 | 3.05E−10 |
| SREK1 | 3.04E−14 | −0.263 | 0.129 | 0.23 | 3.47E−10 |
| GNAI3 | 3.19E−14 | −0.380 | 0.115 | 0.232 | 3.63E−10 |
| VEZT | 3.97E−14 | −0.351 | 0.078 | 0.178 | 4.53E−10 |
| RAB22A | 4.00E−14 | −0.261 | 0.086 | 0.194 | 4.56E−10 |
| WASL | 4.77E−14 | −0.323 | 0.121 | 0.243 | 5.43E−10 |
| ANKRD28 | 4.79E−14 | −0.364 | 0.049 | 0.138 | 5.45E−10 |
| LCOR | 4.86E−14 | −0.375 | 0.062 | 0.155 | 5.54E−10 |
| TMEM223 | 5.07E−14 | −0.252 | 0.064 | 0.162 | 5.78E−10 |
| ATXN2 | 6.09E−14 | −0.320 | 0.067 | 0.168 | 6.94E−10 |
| RAB12 | 6.11E−14 | −0.295 | 0.074 | 0.175 | 6.96E−10 |
| MYH10 | 7.14E−14 | −0.603 | 0.041 | 0.13 | 8.14E−10 |
| MIS18BP1 | 7.41E−14 | −0.387 | 0.084 | 0.185 | 8.45E−10 |
| KIAA1429 | 7.93E−14 | −0.492 | 0.092 | 0.203 | 9.04E−10 |
| LYZ | 8.37E−14 | −0.488 | 0.059 | 0.123 | 9.54E−10 |
| KCTD9 | 1.04E−13 | −0.281 | 0.057 | 0.15 | 1.19E−09 |
| ELL2 | 1.11E−13 | −0.259 | 0.068 | 0.165 | 1.26E−09 |
| EXPH5 | 1.13E−13 | −0.787 | 0.041 | 0.119 | 1.29E−09 |
| KIAA0100 | 1.23E−13 | −0.323 | 0.08 | 0.187 | 1.41E−09 |
| TMED7 | 1.25E−13 | −0.297 | 0.069 | 0.163 | 1.42E−09 |
| NOP14 | 1.33E−13 | −0.543 | 0.059 | 0.156 | 1.52E−09 |
| CETN3 | 1.55E−13 | −0.274 | 0.079 | 0.18 | 1.76E−09 |
| UGDH | 1.84E−13 | −0.261 | 0.101 | 0.195 | 2.10E−09 |
| MED17 | 2.07E−13 | −0.542 | 0.029 | 0.105 | 2.36E−09 |
| TUG1 | 2.09E−13 | −0.381 | 0.102 | 0.219 | 2.39E−09 |
| CHMP1B | 2.32E−13 | −0.394 | 0.084 | 0.189 | 2.64E−09 |
| JUND | 2.36E−13 | −0.257 | 0.061 | 0.156 | 2.69E−09 |
| HOOK3 | 2.92E−13 | −0.458 | 0.071 | 0.176 | 3.33E−09 |
| TIMM8B | 2.99E−13 | −0.364 | 0.118 | 0.216 | 3.41E−09 |
| SNX4 | 3.02E−13 | −0.250 | 0.121 | 0.229 | 3.44E−09 |
| RRP1B | 3.17E−13 | −0.389 | 0.117 | 0.225 | 3.62E−09 |
| TMEM65 | 3.42E−13 | −0.304 | 0.035 | 0.111 | 3.90E−09 |
| RPRD2 | 3.47E−13 | −0.332 | 0.078 | 0.182 | 3.96E−09 |
| TIPARP | 4.11E−13 | −0.491 | 0.064 | 0.163 | 4.69E−09 |
| NFX1 | 4.25E−13 | −0.331 | 0.072 | 0.17 | 4.85E−09 |
| PNPLA8 | 4.75E−13 | −0.438 | 0.089 | 0.201 | 5.41E−09 |
| GABPA | 5.23E−13 | −0.350 | 0.045 | 0.136 | 5.96E−09 |
| LTN1 | 5.58E−13 | −0.468 | 0.13 | 0.244 | 6.37E−09 |
| RNMT | 5.65E−13 | −0.267 | 0.089 | 0.192 | 6.44E−09 |
| CREBZF | 6.13E−13 | −0.281 | 0.057 | 0.141 | 6.99E−09 |
| MRFAP1L1 | 6.19E−13 | −0.464 | 0.082 | 0.179 | 7.05E−09 |
| STAM2 | 7.14E−13 | −0.317 | 0.094 | 0.203 | 8.14E−09 |
| CEP152 | 7.36E−13 | −0.273 | 0.035 | 0.104 | 8.39E−09 |
| UBR4 | 8.08E−13 | −0.356 | 0.064 | 0.147 | 9.21E−09 |
| CTBP1 | 9.00E−13 | −0.302 | 0.029 | 0.105 | 1.03E−08 |
| SERINC3 | 9.13E−13 | −0.263 | 0.145 | 0.253 | 1.04E−08 |
| HELLS | 9.40E−13 | −0.325 | 0.084 | 0.173 | 1.07E−08 |
| ORMDL1 | 9.88E−13 | −0.302 | 0.117 | 0.226 | 1.13E−08 |
| CD3EAP | 1.12E−12 | −0.338 | 0.091 | 0.187 | 1.27E−08 |
| PEG10 | 1.13E−12 | −0.419 | 0.552 | 0.632 | 1.29E−08 |
| MED13L | 1.16E−12 | −0.376 | 0.051 | 0.128 | 1.32E−08 |
| EXOC1 | 1.23E−12 | −0.257 | 0.096 | 0.193 | 1.40E−08 |
| RASA1 | 1.65E−12 | −0.318 | 0.062 | 0.147 | 1.88E−08 |
| PPME1 | 1.70E−12 | −0.260 | 0.061 | 0.147 | 1.94E−08 |
| UBE2D1 | 2.05E−12 | −0.279 | 0.074 | 0.166 | 2.34E−08 |
| RCN2 | 2.12E−12 | −0.330 | 0.095 | 0.202 | 2.42E−08 |
| FEM1B | 2.29E−12 | −0.253 | 0.068 | 0.153 | 2.61E−08 |
| WDR12 | 2.32E−12 | −0.360 | 0.074 | 0.176 | 2.65E−08 |
| ATXN7L3B | 2.39E−12 | −0.439 | 0.113 | 0.224 | 2.72E−08 |
| TRIP11 | 2.80E−12 | −0.291 | 0.156 | 0.259 | 3.19E−08 |
| PLEKHA5 | 2.88E−12 | −0.377 | 0.05 | 0.136 | 3.29E−08 |
| LSG1 | 3.21E−12 | −0.291 | 0.117 | 0.217 | 3.66E−08 |
| TRIM27 | 3.50E−12 | −0.372 | 0.1 | 0.203 | 3.99E−08 |
| REXO2 | 3.58E−12 | −0.349 | 0.118 | 0.229 | 4.08E−08 |
| APPBP2 | 3.92E−12 | −0.302 | 0.081 | 0.171 | 4.47E−08 |
| CCDC43 | 3.99E−12 | −0.316 | 0.083 | 0.182 | 4.54E−08 |
| CRK | 4.23E−12 | −0.490 | 0.047 | 0.133 | 4.82E−08 |
| CCND1 | 4.64E−12 | −0.470 | 0.117 | 0.2 | 5.28E−08 |
| PAFAH1B2 | 4.78E−12 | −0.373 | 0.112 | 0.225 | 5.45E−08 |
| ZBTB44 | 5.56E−12 | −0.397 | 0.043 | 0.126 | 6.34E−08 |
| NRAS | 5.66E−12 | −0.475 | 0.064 | 0.159 | 6.45E−08 |
| VRK1 | 5.93E−12 | −0.515 | 0.084 | 0.176 | 6.76E−08 |
| MYNN | 6.03E−12 | −0.261 | 0.137 | 0.241 | 6.87E−08 |
| SRP54 | 6.69E−12 | −0.301 | 0.146 | 0.252 | 7.63E−08 |
| CSNK2A1 | 7.29E−12 | −0.292 | 0.1 | 0.197 | 8.31E−08 |
| SRA1 | 7.68E−12 | −0.304 | 0.059 | 0.134 | 8.75E−08 |
| ASPH | 8.66E−12 | −0.387 | 0.143 | 0.246 | 9.88E−08 |
| SEC23IP | 9.70E−12 | −0.266 | 0.048 | 0.129 | 1.11E−07 |
| ARL6IP5 | 1.02E−11 | −0.370 | 0.51 | 0.6 | 1.16E−07 |
| PCYT1A | 1.03E−11 | −0.357 | 0.08 | 0.179 | 1.17E−07 |
| SCAMP2 | 1.03E−11 | −0.284 | 0.043 | 0.125 | 1.18E−07 |
| ING2 | 1.38E−11 | −0.320 | 0.047 | 0.132 | 1.58E−07 |
| TMEM192 | 1.40E−11 | −0.304 | 0.05 | 0.136 | 1.59E−07 |
| TMED5 | 1.54E−11 | −0.270 | 0.094 | 0.194 | 1.76E−07 |
| TRIB1 | 1.56E−11 | −0.352 | 0.079 | 0.169 | 1.78E−07 |
| KIAA1586 | 1.62E−11 | −0.271 | 0.078 | 0.173 | 1.85E−07 |
| LYAR | 2.01E−11 | −0.463 | 0.073 | 0.16 | 2.29E−07 |
| KIF20B | 2.22E−11 | −0.480 | 0.079 | 0.16 | 2.53E−07 |
| LNPEP | 2.34E−11 | −0.350 | 0.06 | 0.141 | 2.67E−07 |
| DDX10 | 2.64E−11 | −0.519 | 0.064 | 0.151 | 3.01E−07 |
| ASPM | 2.71E−11 | −0.302 | 0.161 | 0.109 | 3.09E−07 |
| SRSF6 | 2.74E−11 | −0.329 | 0.106 | 0.204 | 3.12E−07 |
| CEP78 | 2.84E−11 | −0.270 | 0.061 | 0.141 | 3.24E−07 |
| ACADVL | 3.29E−11 | −0.276 | 0.054 | 0.136 | 3.76E−07 |
| CTTNBP2NL | 3.72E−11 | −0.263 | 0.047 | 0.128 | 4.24E−07 |
| KIFAP3 | 4.05E−11 | −0.269 | 0.068 | 0.159 | 4.62E−07 |
| FGFR1OP2 | 4.13E−11 | −0.550 | 0.124 | 0.231 | 4.71E−07 |
| AGO2 | 4.30E−11 | −0.342 | 0.039 | 0.115 | 4.91E−07 |
| PBRM1 | 4.38E−11 | −0.426 | 0.081 | 0.162 | 4.99E−07 |
| FAM98B | 5.13E−11 | −0.340 | 0.105 | 0.206 | 5.85E−07 |
| C2CD5 | 6.00E−11 | −0.373 | 0.04 | 0.118 | 6.84E−07 |
| BMP2K | 6.04E−11 | −0.368 | 0.033 | 0.107 | 6.89E−07 |
| GNPDA1 | 6.59E−11 | −0.357 | 0.096 | 0.196 | 7.51E−07 |
| SRPR | 6.90E−11 | −0.345 | 0.082 | 0.176 | 7.87E−07 |
| SIPA1L1 | 7.19E−11 | −0.280 | 0.037 | 0.11 | 8.19E−07 |
| ORC4 | 7.83E−11 | −0.258 | 0.084 | 0.181 | 8.93E−07 |
| ADD1 | 9.58E−11 | −0.305 | 0.046 | 0.125 | 1.09E−06 |
| PRR14L | 9.67E−11 | −0.265 | 0.1 | 0.194 | 1.10E−06 |
| WDR36 | 9.97E−11 | −0.353 | 0.062 | 0.15 | 1.14E−06 |
| GS1-251I9.4 | 1.10E−10 | −0.599 | 0.078 | 0.15 | 1.25E−06 |
| NOA1 | 1.10E−10 | −0.305 | 0.062 | 0.149 | 1.25E−06 |
| MRPS25 | 1.13E−10 | −0.254 | 0.07 | 0.157 | 1.29E−06 |
| TXNDC16 | 1.18E−10 | −0.337 | 0.062 | 0.149 | 1.35E−06 |
| MTMR2 | 1.20E−10 | −0.498 | 0.041 | 0.113 | 1.36E−06 |
| FAM8A1 | 1.38E−10 | −0.360 | 0.04 | 0.117 | 1.58E−06 |
| ECT2 | 1.42E−10 | −0.414 | 0.176 | 0.267 | 1.62E−06 |
| DNM2 | 1.53E−10 | −0.352 | 0.045 | 0.124 | 1.74E−06 |
| ZNF260 | 1.78E−10 | −0.460 | 0.077 | 0.168 | 2.02E−06 |
| RPP40 | 1.88E−10 | −0.342 | 0.05 | 0.127 | 2.14E−06 |
| UBR2 | 1.98E−10 | −0.296 | 0.06 | 0.141 | 2.25E−06 |
| WDHD1 | 2.45E−10 | −0.441 | 0.047 | 0.124 | 2.79E−06 |
| USP33 | 2.70E−10 | −0.382 | 0.076 | 0.164 | 3.07E−06 |
| LTV1 | 2.72E−10 | −0.400 | 0.114 | 0.21 | 3.10E−06 |
| AGTPBP1 | 4.08E−10 | −0.305 | 0.051 | 0.129 | 4.65E−06 |
| NRBP1 | 4.44E−10 | −0.291 | 0.109 | 0.189 | 5.06E−06 |
| RHOB | 4.45E−10 | −0.286 | 0.107 | 0.193 | 5.07E−06 |
| ASUN | 4.95E−10 | −0.334 | 0.093 | 0.186 | 5.65E−06 |
| UBAP1 | 5.67E−10 | −0.291 | 0.068 | 0.155 | 6.47E−06 |
| TRMT5 | 5.78E−10 | −0.332 | 0.085 | 0.167 | 6.58E−06 |
| RBM5 | 6.21E−10 | −0.254 | 0.079 | 0.156 | 7.08E−06 |
| UTP20 | 6.65E−10 | −0.324 | 0.056 | 0.138 | 7.58E−06 |
| PKN2 | 6.68E−10 | −0.433 | 0.097 | 0.19 | 7.62E−06 |
| FER | 7.05E−10 | −0.449 | 0.048 | 0.119 | 8.03E−06 |
| ZCCHC8 | 7.16E−10 | −0.274 | 0.059 | 0.141 | 8.16E−06 |
| INTS8 | 7.26E−10 | −0.351 | 0.076 | 0.168 | 8.28E−06 |
| MRPS28 | 8.83E−10 | −0.337 | 0.109 | 0.177 | 1.01E−05 |
| SAFB2 | 9.00E−10 | −0.272 | 0.067 | 0.15 | 1.03E−05 |
| RUFY2 | 9.98E−10 | −0.346 | 0.039 | 0.101 | 1.14E−05 |
| TLK1 | 1.01E−09 | −0.298 | 0.106 | 0.186 | 1.15E−05 |
| MAP9 | 1.02E−09 | −0.312 | 0.063 | 0.135 | 1.16E−05 |
| NFKB1 | 1.02E−09 | −0.374 | 0.053 | 0.122 | 1.17E−05 |
| KIF2A | 1.10E−09 | −0.450 | 0.097 | 0.186 | 1.25E−05 |
| FMNL2 | 1.36E−09 | −0.257 | 0.043 | 0.114 | 1.55E−05 |
| PDP1 | 1.58E−09 | −0.481 | 0.049 | 0.105 | 1.80E−05 |
| DUSP12 | 1.69E−09 | −0.388 | 0.047 | 0.116 | 1.92E−05 |
| AP3B1 | 1.71E−09 | −0.396 | 0.086 | 0.171 | 1.95E−05 |
| SHPRH | 1.90E−09 | −0.432 | 0.045 | 0.119 | 2.16E−05 |
| HEXIM1 | 1.93E−09 | −0.368 | 0.05 | 0.115 | 2.20E−05 |
| TRMT11 | 2.03E−09 | −0.313 | 0.068 | 0.148 | 2.31E−05 |
| LRRCC1 | 2.11E−09 | −0.375 | 0.092 | 0.181 | 2.40E−05 |
| ZNF326 | 2.16E−09 | −0.402 | 0.094 | 0.181 | 2.46E−05 |
| ARFGEF2 | 2.23E−09 | −0.276 | 0.094 | 0.171 | 2.54E−05 |
| ATP13A3 | 2.25E−09 | −0.450 | 0.101 | 0.195 | 2.56E−05 |
| IQCG | 2.52E−09 | −0.259 | 0.045 | 0.109 | 2.87E−05 |
| NDRG1 | 2.71E−09 | −0.435 | 0.058 | 0.13 | 3.08E−05 |
| GRWD1 | 2.76E−09 | −0.326 | 0.073 | 0.155 | 3.15E−05 |
| SLC20A1 | 2.97E−09 | −0.437 | 0.059 | 0.137 | 3.39E−05 |
| RLIM | 3.48E−09 | −0.358 | 0.072 | 0.154 | 3.96E−05 |
| EPC2 | 4.03E−09 | −0.253 | 0.047 | 0.113 | 4.59E−05 |
| ZBTB1 | 4.13E−09 | −0.282 | 0.073 | 0.157 | 4.70E−05 |
| CNOT10 | 4.20E−09 | −0.335 | 0.065 | 0.144 | 4.79E−05 |
| RCOR1 | 4.56E−09 | −0.251 | 0.052 | 0.126 | 5.19E−05 |
| RND3 | 4.86E−09 | −0.425 | 0.127 | 0.106 | 5.54E−05 |
| DNTTIP1 | 5.48E−09 | −0.254 | 0.065 | 0.137 | 6.25E−05 |
| IMPAD1 | 5.54E−09 | −0.423 | 0.056 | 0.13 | 6.31E−05 |
| FBXO21 | 5.93E−09 | −0.413 | 0.066 | 0.143 | 6.76E−05 |
| CEP44 | 6.71E−09 | −0.315 | 0.046 | 0.116 | 7.65E−05 |
| LEMD3 | 7.38E−09 | −0.273 | 0.047 | 0.112 | 8.41E−05 |
| VCAN | 7.54E−09 | −0.736 | 0.044 | 0.104 | 8.59E−05 |
| PAQR3 | 8.93E−09 | −0.399 | 0.037 | 0.102 | 1.02E−04 |
| COX15 | 9.82E−09 | −0.333 | 0.058 | 0.135 | 1.12E−04 |
| WDR75 | 1.02E−08 | −0.306 | 0.084 | 0.17 | 1.16E−04 |
| UBR3 | 1.04E−08 | −0.369 | 0.06 | 0.135 | 1.19E−04 |
| DR1 | 1.08E−08 | −0.298 | 0.079 | 0.163 | 1.23E−04 |
| YLPM1 | 1.09E−08 | −0.469 | 0.05 | 0.117 | 1.24E−04 |
| MPP6 | 1.12E−08 | −0.271 | 0.042 | 0.109 | 1.28E−04 |
| BTBD1 | 1.14E−08 | −0.373 | 0.038 | 0.102 | 1.30E−04 |
| CDC40 | 1.40E−08 | −0.305 | 0.099 | 0.183 | 1.60E−04 |
| GTF3C1 | 1.47E−08 | −0.289 | 0.051 | 0.12 | 1.67E−04 |
| SOCS4 | 1.52E−08 | −0.448 | 0.084 | 0.16 | 1.74E−04 |
| CWC22 | 1.98E−08 | −0.267 | 0.097 | 0.182 | 2.26E−04 |
| ETFA | 2.36E−08 | −0.286 | 0.097 | 0.174 | 2.69E−04 |
| GPN1 | 2.45E−08 | −0.297 | 0.064 | 0.135 | 2.79E−04 |
| PPWD1 | 2.55E−08 | −0.292 | 0.061 | 0.129 | 2.91E−04 |
| POLR3D | 2.87E−08 | −0.316 | 0.052 | 0.118 | 3.27E−04 |
| CEP57 | 2.97E−08 | −0.490 | 0.071 | 0.143 | 3.39E−04 |
| PYGB | 3.20E−08 | −0.273 | 0.043 | 0.107 | 3.64E−04 |
| CERS6 | 3.37E−08 | −0.543 | 0.079 | 0.136 | 3.84E−04 |
| AASDHPPT | 5.90E−08 | −0.310 | 0.084 | 0.165 | 6.72E−04 |
| RRS1 | 6.48E−08 | −0.328 | 0.043 | 0.104 | 7.39E−04 |
| ABCF2 | 9.95E−08 | −0.320 | 0.055 | 0.12 | 1.13E−03 |
| XPO7 | 1.06E−07 | −0.393 | 0.051 | 0.109 | 1.20E−03 |
| ACADM | 1.07E−07 | −0.387 | 0.119 | 0.191 | 1.22E−03 |
| RBM28 | 1.11E−07 | −0.348 | 0.09 | 0.164 | 1.27E−03 |
| SNAPC3 | 1.20E−07 | −0.294 | 0.051 | 0.115 | 1.37E−03 |
| BTBD7 | 1.26E−07 | −0.340 | 0.056 | 0.124 | 1.44E−03 |
| AGPAT5 | 1.28E−07 | −0.360 | 0.105 | 0.19 | 1.46E−03 |
| SC5D | 1.31E−07 | −0.286 | 0.066 | 0.133 | 1.49E−03 |
| MKLN1 | 1.50E−07 | −0.331 | 0.085 | 0.162 | 1.71E−03 |
| APC | 1.55E−07 | −0.359 | 0.056 | 0.124 | 1.77E−03 |
| TDP2 | 1.89E−07 | −0.330 | 0.053 | 0.118 | 2.15E−03 |
| C12orf4 | 2.12E−07 | −0.334 | 0.05 | 0.111 | 2.42E−03 |
| SPIDR | 2.15E−07 | −0.374 | 0.045 | 0.105 | 2.45E−03 |
| HCG18 | 2.50E−07 | −0.279 | 0.048 | 0.112 | 2.85E−03 |
| METTL8 | 3.86E−07 | −0.264 | 0.043 | 0.103 | 4.40E−03 |
| UPF3B | 4.39E−07 | −0.444 | 0.08 | 0.152 | 5.00E−03 |
| MYBBP1A | 5.57E−07 | −0.401 | 0.059 | 0.114 | 6.34E−03 |
| DTNBP1 | 5.86E−07 | −0.313 | 0.068 | 0.133 | 6.68E−03 |
| DIMT1 | 6.80E−07 | −0.278 | 0.056 | 0.122 | 7.75E−03 |
| CUL4B | 7.81E−07 | −0.275 | 0.072 | 0.141 | 8.90E−03 |
| C11orf54 | 8.70E−07 | −0.387 | 0.07 | 0.127 | 9.92E−03 |
| TMED1 | 1.19E−06 | −0.257 | 0.048 | 0.104 | 1.36E−02 |
| ACAT1 | 2.15E−06 | −0.271 | 0.093 | 0.167 | 2.45E−02 |
| MED29 | 3.26E−06 | −0.293 | 0.076 | 0.139 | 3.71E−02 |
| OTUD6B | 3.28E−06 | −0.404 | 0.049 | 0.105 | 3.74E−02 |
| RBM12 | 4.27E−06 | −0.272 | 0.06 | 0.121 | 4.87E−02 |

CSF_full.r2.setRvNR.tumor.pre.bimod.markers.dn.1

| | | | | | |
|---|---|---|---|---|---|
| MTRNR2L2 | 5.24E−223 | −3.166 | 0.306 | 0.93 | 5.98E−219 |
| MTRNR2L12 | 2.37E−173 | −2.847 | 0.266 | 0.867 | 2.70E−169 |
| RNA18S5 | 1.72E−134 | −3.207 | 0.005 | 0.564 | 1.96E−130 |
| MTRNR2L8 | 7.55E−131 | −2.804 | 0.396 | 0.813 | 8.61E−127 |
| S100A6 | 2.18E−128 | −1.989 | 0.433 | 0.827 | 2.49E−124 |
| GSTP1 | 9.40E−110 | −1.939 | 0.038 | 0.567 | 1.07E−105 |
| HAP1 | 2.24E−103 | −1.832 | 0.022 | 0.507 | 2.55E−99 |
| SNHG5 | 1.29E−95 | −1.829 | 0.431 | 0.67 | 1.47E−91 |
| MFGE8 | 1.75E−95 | −2.865 | 0.01 | 0.452 | 1.99E−91 |
| CTC-338M12.5 | 1.11E−94 | −3.097 | 0.051 | 0.484 | 1.26E−90 |
| TMSB10 | 2.04E−93 | −1.341 | 0.835 | 0.952 | 2.33E−89 |
| MTRNR2L3 | 5.99E−88 | −1.840 | 0.401 | 0.731 | 6.82E−84 |
| THBS1 | 7.95E−83 | −2.884 | 0.036 | 0.463 | 9.06E−79 |
| MTRNR2L6 | 4.47E−81 | −2.085 | 0.101 | 0.548 | 5.09E−77 |
| DEK | 3.82E−75 | −1.433 | 0.374 | 0.662 | 4.35E−71 |
| YWHAZ | 5.84E−73 | −1.185 | 0.571 | 0.725 | 6.65E−69 |
| LDHB | 1.22E−71 | −1.743 | 0.055 | 0.469 | 1.39E−67 |
| PABPC1 | 4.43E−71 | −0.919 | 0.965 | 0.99 | 5.05E−67 |
| IFI27 | 2.24E−70 | −2.569 | 0.071 | 0.399 | 2.55E−66 |
| LGALS1 | 2.66E−69 | −1.591 | 0.29 | 0.636 | 3.04E−65 |
| SDC2 | 4.50E−69 | −1.899 | 0.005 | 0.322 | 5.13E−65 |
| YWHAE | 8.63E−66 | −1.237 | 0.437 | 0.604 | 9.84E−62 |
| NDUFA4 | 1.34E−65 | −1.072 | 0.648 | 0.737 | 1.53E−61 |
| ARPC5 | 5.02E−63 | −1.728 | 0.29 | 0.596 | 5.73E−59 |
| SNHG6 | 6.57E−63 | −1.326 | 0.385 | 0.478 | 7.49E−59 |
| HIST1H4C | 3.66E−62 | −1.673 | 0.403 | 0.678 | 4.17E−58 |
| TUBA1A | 4.78E−62 | −1.827 | 0.13 | 0.434 | 5.45E−58 |
| TMSB4X | 1.00E−61 | −1.207 | 0.518 | 0.772 | 1.14E−57 |
| KRT7 | 3.83E−61 | −1.219 | 0.355 | 0.577 | 4.37E−57 |
| SLC38A1 | 1.82E−58 | −1.427 | 0.282 | 0.541 | 2.07E−54 |
| UBL5 | 9.44E−56 | −1.111 | 0.534 | 0.61 | 1.08E−51 |
| CD59 | 1.04E−55 | −1.489 | 0.284 | 0.512 | 1.18E−51 |
| ARF1 | 2.78E−54 | −1.279 | 0.425 | 0.62 | 3.17E−50 |
| SLC38A2 | 2.95E−53 | −1.459 | 0.285 | 0.477 | 3.36E−49 |
| APP | 3.52E−53 | −1.141 | 0.363 | 0.541 | 4.02E−49 |
| TM9SF2 | 4.66E−53 | −1.258 | 0.344 | 0.576 | 5.31E−49 |
| ACTB | 1.73E−51 | −0.934 | 0.621 | 0.738 | 1.97E−47 |
| ANXA1 | 2.13E−51 | −1.687 | 0.082 | 0.418 | 2.43E−47 |
| FSTL1 | 2.42E−51 | −1.427 | 0.006 | 0.269 | 2.76E−47 |
| HNRNPA2B1 | 3.79E−51 | −0.848 | 0.632 | 0.779 | 4.32E−47 |
| MCM4 | 3.24E−50 | −1.392 | 0.195 | 0.393 | 3.70E−46 |
| CCNI | 6.90E−49 | −0.949 | 0.426 | 0.545 | 7.86E−45 |
| STMN1 | 7.62E−49 | −1.166 | 0.293 | 0.564 | 8.69E−45 |
| CBX3 | 1.17E−48 | −0.896 | 0.534 | 0.667 | 1.33E−44 |
| ATP5J | 1.28E−48 | −1.125 | 0.261 | 0.461 | 1.46E−44 |
| TAGLN2 | 4.40E−48 | −1.153 | 0.306 | 0.535 | 5.01E−44 |
| COX7A2 | 3.65E−47 | −0.850 | 0.569 | 0.667 | 4.16E−43 |
| MTRNR2L11 | 7.39E−47 | −1.502 | 0.124 | 0.465 | 8.42E−43 |
| NDUFA13 | 2.02E−45 | −0.898 | 0.428 | 0.523 | 2.30E−41 |
| TMEM70 | 2.03E−45 | −1.345 | 0.176 | 0.386 | 2.31E−41 |
| PFN1 | 8.45E−45 | −0.791 | 0.531 | 0.629 | 9.63E−41 |
| PPP1CB | 1.89E−43 | −1.208 | 0.322 | 0.499 | 2.15E−39 |
| MTRNR2L7 | 5.41E−43 | −1.622 | 0.046 | 0.317 | 6.16E−39 |
| IGFBP3 | 1.71E−42 | −1.600 | 0.002 | 0.213 | 1.94E−38 |
| CACYBP | 2.07E−42 | −1.253 | 0.282 | 0.475 | 2.36E−38 |
| AZIN1 | 2.69E−42 | −1.013 | 0.206 | 0.398 | 3.07E−38 |
| MYL6 | 3.79E−42 | −0.813 | 0.65 | 0.713 | 4.32E−38 |
| PRKDC | 5.53E−42 | −1.069 | 0.358 | 0.466 | 6.30E−38 |
| OCIAD2 | 5.69E−42 | −1.166 | 0.143 | 0.393 | 6.49E−38 |
| CD47 | 1.04E−41 | −1.514 | 0.139 | 0.412 | 1.19E−37 |
| B2M | 1.43E−41 | −0.497 | 0.919 | 0.718 | 1.63E−37 |
| MTRNR2L5 | 2.09E−41 | −1.349 | 0.041 | 0.307 | 2.38E−37 |
| CENPF | 2.50E−41 | −1.611 | 0.17 | 0.455 | 2.85E−37 |
| MT-CO2 | 1.89E−40 | −1.167 | 0.735 | 0.806 | 2.15E−36 |
| HMGB1 | 7.23E−40 | −1.087 | 0.445 | 0.601 | 8.25E−36 |
| MIF | 9.40E−40 | −1.269 | 0.312 | 0.472 | 1.07E−35 |
| IPO5 | 1.04E−39 | −1.198 | 0.176 | 0.42 | 1.19E−35 |
| NUCKS1 | 1.19E−39 | −0.973 | 0.607 | 0.655 | 1.35E−35 |
| CDKN2A | 1.24E−39 | −1.172 | 0.089 | 0.37 | 1.42E−35 |
| KLK6 | 1.67E−39 | −1.648 | 0.022 | 0.247 | 1.91E−35 |
| C3 | 1.81E−39 | −1.657 | 0.024 | 0.241 | 2.06E−35 |
| MT-CYB | 6.30E−39 | −0.968 | 0.732 | 0.8 | 7.18E−35 |
| SSR4 | 1.88E−38 | −1.154 | 0.466 | 0.547 | 2.14E−34 |
| UBA52 | 3.56E−38 | −0.579 | 0.756 | 0.711 | 4.06E−34 |
| ILF2 | 7.27E−38 | −1.090 | 0.211 | 0.458 | 8.29E−34 |
| TIMM8B | 4.08E−37 | −1.047 | 0.239 | 0.322 | 4.65E−33 |
| CNIH4 | 5.08E−37 | −1.159 | 0.252 | 0.431 | 5.79E−33 |
| TPM3 | 8.06E−37 | −1.074 | 0.415 | 0.563 | 9.18E−33 |
| ANLN | 1.34E−36 | −1.210 | 0.089 | 0.275 | 1.53E−32 |
| IFITM3 | 1.91E−36 | −1.415 | 0.13 | 0.288 | 2.17E−32 |
| SAT1 | 4.03E−36 | −1.048 | 0.334 | 0.512 | 4.59E−32 |
| UCHL1 | 6.45E−36 | −1.057 | 0.006 | 0.205 | 7.35E−32 |
| ANP32B | 1.86E−35 | −0.887 | 0.387 | 0.497 | 2.12E−31 |
| TUBB | 2.31E−35 | −0.972 | 0.455 | 0.56 | 2.64E−31 |
| TPM1 | 8.70E−35 | −0.733 | 0.488 | 0.582 | 9.91E−31 |
| YBX3 | 8.94E−35 | −1.107 | 0.151 | 0.368 | 1.02E−30 |
| PEG10 | 1.03E−34 | −0.879 | 0.282 | 0.32 | 1.17E−30 |
| SYNCRIP | 3.24E−34 | −0.892 | 0.263 | 0.484 | 3.70E−30 |
| ACTN4 | 6.41E−34 | −1.049 | 0.255 | 0.452 | 7.31E−30 |
| CYCS | 1.34E−33 | −0.932 | 0.334 | 0.538 | 1.52E−29 |
| HSD17B12 | 1.46E−33 | −1.113 | 0.254 | 0.402 | 1.66E−29 |
| NDUFS6 | 3.22E−33 | −1.064 | 0.363 | 0.48 | 3.67E−29 |
| TUBA1B | 4.33E−33 | −0.857 | 0.339 | 0.515 | 4.93E−29 |
| GUK1 | 4.64E−33 | −1.117 | 0.311 | 0.463 | 5.28E−29 |
| S100A13 | 5.15E−33 | −1.141 | 0.144 | 0.368 | 5.88E−29 |
| RPL27 | 7.27E−33 | −0.631 | 0.867 | 0.776 | 8.29E−29 |
| ATAD2 | 8.88E−33 | −1.225 | 0.128 | 0.325 | 1.01E−28 |
| CTNNB1 | 9.71E−33 | −1.056 | 0.293 | 0.428 | 1.11E−28 |
| S100A11 | 1.53E−32 | −0.755 | 0.618 | 0.674 | 1.74E−28 |
| OAZ1 | 1.62E−32 | −0.761 | 0.512 | 0.545 | 1.85E−28 |
| 11-Sep | 1.95E−32 | −1.154 | 0.163 | 0.346 | 2.22E−28 |
| TMPO | 2.37E−32 | −1.155 | 0.105 | 0.326 | 2.70E−28 |
| S100A1 | 2.62E−32 | −1.262 | 0.008 | 0.178 | 2.99E−28 |
| TMEM123 | 3.08E−32 | −0.795 | 0.45 | 0.5 | 3.51E−28 |
| TFDP1 | 3.74E−32 | −1.074 | 0.067 | 0.276 | 4.26E−28 |
| IDH2 | 6.37E−32 | −1.093 | 0.239 | 0.32 | 7.26E−28 |
| TRAM1 | 1.07E−31 | −0.896 | 0.279 | 0.433 | 1.22E−27 |
| DNMT1 | 2.57E−31 | −1.138 | 0.163 | 0.367 | 2.92E−27 |
| RAN | 3.92E−31 | −0.752 | 0.398 | 0.501 | 4.47E−27 |
| HMGN1 | 6.04E−31 | −0.763 | 0.458 | 0.542 | 6.88E−27 |
| FXYD5 | 6.52E−31 | −0.988 | 0.036 | 0.259 | 7.43E−27 |
| ATP5L | 7.08E−31 | −0.571 | 0.523 | 0.48 | 8.07E−27 |
| ENY2 | 8.22E−31 | −0.774 | 0.415 | 0.506 | 9.37E−27 |
| CALM1 | 9.41E−31 | −0.671 | 0.517 | 0.639 | 1.07E−26 |
| PLAGL1 | 1.61E−30 | −0.856 | 0.017 | 0.186 | 1.83E−26 |
| CCT5 | 2.55E−30 | −0.887 | 0.418 | 0.542 | 2.91E−26 |
| MGST3 | 3.62E−30 | −1.058 | 0.216 | 0.387 | 4.12E−26 |
| MTRNR2L13 | 4.80E−30 | −1.313 | 0.063 | 0.284 | 5.47E−26 |
| C12orf57 | 5.79E−30 | −0.638 | 0.425 | 0.406 | 6.60E−26 |
| YBX1 | 7.67E−30 | −0.905 | 0.504 | 0.611 | 8.74E−26 |
| ANP32E | 1.22E−29 | −1.050 | 0.079 | 0.316 | 1.39E−25 |
| CD63 | 1.39E−29 | −0.903 | 0.382 | 0.487 | 1.58E−25 |
| VIM | 2.87E−29 | −0.956 | 0.089 | 0.346 | 3.27E−25 |
| CAPN2 | 3.08E−29 | −1.085 | 0.216 | 0.349 | 3.51E−25 |
| PAICS | 3.49E−29 | −0.960 | 0.282 | 0.401 | 3.98E−25 |
| BTG3 | 7.43E−29 | −1.099 | 0.054 | 0.26 | 8.47E−25 |
| RPS16 | 1.85E−28 | −0.329 | 0.943 | 0.823 | 2.10E−24 |
| CFI | 3.26E−28 | −1.196 | 0.002 | 0.149 | 3.71E−24 |
| CKB | 3.29E−28 | −1.146 | 0.062 | 0.243 | 3.75E−24 |
| ENAH | 3.40E−28 | −0.631 | 0.266 | 0.224 | 3.88E−24 |
| SET | 6.44E−28 | −0.778 | 0.483 | 0.548 | 7.34E−24 |
| S100A10 | 2.43E−27 | −0.885 | 0.345 | 0.44 | 2.77E−23 |
| NFIB | 3.40E−27 | −0.906 | 0.257 | 0.499 | 3.87E−23 |
| IER3 | 4.17E−27 | −0.774 | 0.349 | 0.319 | 4.76E−23 |
| ITGB8 | 5.39E−27 | −1.050 | 0.049 | 0.265 | 6.14E−23 |
| EIF5 | 5.86E−27 | −0.640 | 0.415 | 0.474 | 6.68E−23 |
| SMC4 | 6.61E−27 | −1.048 | 0.225 | 0.427 | 7.53E−23 |
| CD109 | 9.44E−27 | −0.926 | 0.019 | 0.178 | 1.08E−22 |
| LGALS3BP | 1.11E−26 | −1.274 | 0.044 | 0.208 | 1.27E−22 |
| SQLE | 1.79E−26 | −0.996 | 0.328 | 0.475 | 2.04E−22 |
| B3GNT5 | 2.47E−26 | −0.842 | 0.003 | 0.148 | 2.82E−22 |
| RPL9 | 3.04E−26 | −0.651 | 0.578 | 0.577 | 3.47E−22 |
| CETN2 | 3.57E−26 | −0.353 | 0.292 | 0.175 | 4.07E−22 |
| RBM8A | 3.78E−26 | −0.834 | 0.193 | 0.368 | 4.31E−22 |
| SNRPD1 | 4.65E−26 | −0.826 | 0.306 | 0.408 | 5.30E−22 |
| VIMP | 5.13E−26 | −1.013 | 0.252 | 0.348 | 5.85E−22 |
| SLC44A2 | 7.32E−26 | −0.992 | 0.081 | 0.288 | 8.35E−22 |
| ACTN1 | 1.05E−25 | −0.951 | 0.147 | 0.376 | 1.20E−21 |
| FAM126A | 1.73E−25 | −0.739 | 0.01 | 0.164 | 1.97E−21 |
| HSPA8 | 1.84E−25 | −0.608 | 0.502 | 0.525 | 2.10E−21 |
| PRICKLE1 | 2.15E−25 | −0.734 | 0.002 | 0.136 | 2.45E−21 |
| IGFBP7 | 2.54E−25 | −0.767 | 0.003 | 0.142 | 2.89E−21 |
| MTRNR2L1 | 2.67E−25 | −0.725 | 0.932 | 0.927 | 3.04E−21 |
| HNRNPA1 | 3.91E−25 | −0.451 | 0.485 | 0.423 | 4.46E−21 |
| IGF2BP3 | 4.05E−25 | −0.689 | 0.002 | 0.135 | 4.62E−21 |
| CHCHD2 | 5.47E−25 | −0.721 | 0.393 | 0.453 | 6.23E−21 |
| LPL | 5.94E−25 | −0.763 | 0.005 | 0.135 | 6.77E−21 |
| AHR | 1.54E−24 | −1.083 | 0.094 | 0.295 | 1.75E−20 |
| MRPL13 | 1.95E−24 | −0.830 | 0.3 | 0.401 | 2.22E−20 |
| PSMB3 | 2.18E−24 | −0.978 | 0.152 | 0.319 | 2.49E−20 |
| RAB34 | 2.30E−24 | −0.654 | 0.041 | 0.151 | 2.62E−20 |
| TUBA1C | 2.48E−24 | −0.914 | 0.214 | 0.333 | 2.83E−20 |
| COX7B | 2.65E−24 | −0.693 | 0.407 | 0.453 | 3.02E−20 |
| TPM2 | 2.66E−24 | −0.625 | 0.002 | 0.129 | 3.03E−20 |
| MRAS | 2.74E−24 | −0.891 | 0.003 | 0.115 | 3.12E−20 |
| TPI1 | 3.26E−24 | −0.474 | 0.528 | 0.551 | 3.71E−20 |
| APLP2 | 3.27E−24 | −0.731 | 0.426 | 0.49 | 3.73E−20 |
| COX6A1P2 | 3.79E−24 | −0.969 | 0.177 | 0.273 | 4.32E−20 |
| IFRD2 | 4.86E−24 | −1.077 | 0.087 | 0.218 | 5.54E−20 |
| SMURF2 | 7.11E−24 | −0.963 | 0.04 | 0.218 | 8.11E−20 |
| EHF | 7.14E−24 | −0.378 | 0.182 | 0.113 | 8.14E−20 |
| PABPC3 | 7.52E−24 | −0.889 | 0.174 | 0.333 | 8.57E−20 |
| MB21D1 | 8.49E−24 | −0.699 | 0.006 | 0.146 | 9.67E−20 |
| MT-ATP6 | 1.06E−23 | −0.908 | 0.732 | 0.754 | 1.21E−19 |
| PDCD10 | 1.26E−23 | −0.832 | 0.295 | 0.443 | 1.44E−19 |
| MOXD1 | 1.52E−23 | −0.530 | 0 | 0.117 | 1.73E−19 |
| IFITM1 | 1.65E−23 | −1.167 | 0.022 | 0.173 | 1.88E−19 |
| MTDH | 1.75E−23 | −0.492 | 0.483 | 0.437 | 2.00E−19 |
| UBE2V2 | 2.91E−23 | −0.745 | 0.284 | 0.349 | 3.32E−19 |
| GPX1 | 3.20E−23 | −0.817 | 0.258 | 0.383 | 3.65E−19 |
| NDUFS5 | 3.58E−23 | −0.627 | 0.433 | 0.528 | 4.08E−19 |
| ID2 | 4.03E−23 | −0.930 | 0.281 | 0.401 | 4.59E−19 |
| PTTG1IP | 5.65E−23 | −0.756 | 0.422 | 0.516 | 6.44E−19 |
| DSC2 | 9.61E−23 | −0.793 | 0.116 | 0.203 | 1.10E−18 |
| PAPOLA | 1.72E−22 | −0.610 | 0.385 | 0.456 | 1.97E−18 |
| PHLDA1 | 1.88E−22 | −0.904 | 0.029 | 0.194 | 2.14E−18 |
| TXN | 1.92E−22 | −0.530 | 0.591 | 0.596 | 2.19E−18 |
| GPNMB | 2.25E−22 | −0.716 | 0.081 | 0.273 | 2.56E−18 |
| FAM60A | 2.29E−22 | −0.810 | 0.139 | 0.237 | 2.61E−18 |
| PCDH18 | 2.57E−22 | −0.599 | 0 | 0.111 | 2.93E−18 |
| CDH11 | 2.57E−22 | −0.635 | 0 | 0.111 | 2.93E−18 |
| RBP1 | 3.01E−22 | −0.838 | 0.032 | 0.102 | 3.43E−18 |
| PM20D2 | 3.26E−22 | −0.648 | 0.019 | 0.173 | 3.72E−18 |
| FLNA | 3.40E−22 | −0.950 | 0.139 | 0.327 | 3.88E−18 |
| ABRACL | 3.70E−22 | −0.908 | 0.162 | 0.349 | 4.22E−18 |
| H2AFZ | 4.13E−22 | −0.853 | 0.357 | 0.442 | 4.70E−18 |
| MAP1B | 5.50E−22 | −0.898 | 0.048 | 0.222 | 6.27E−18 |
| NDUFB9 | 5.78E−22 | −0.576 | 0.532 | 0.599 | 6.58E−18 |
| C17orf76-AS1 | 6.72E−22 | −0.295 | 0.629 | 0.506 | 7.66E−18 |
| PDCD5 | 8.92E−22 | −0.843 | 0.239 | 0.392 | 1.02E−17 |
| ILF3 | 1.04E−21 | −0.684 | 0.352 | 0.382 | 1.19E−17 |
| LMNA | 1.07E−21 | −0.819 | 0.33 | 0.415 | 1.22E−17 |
| SLC25A5 | 1.23E−21 | −0.493 | 0.464 | 0.444 | 1.40E−17 |
| CMTM7 | 1.51E−21 | −0.660 | 0.002 | 0.117 | 1.72E−17 |
| C19orf53 | 1.59E−21 | −0.606 | 0.352 | 0.402 | 1.81E−17 |
| ENO1 | 1.72E−21 | −0.521 | 0.615 | 0.636 | 1.96E−17 |
| RND3 | 1.80E−21 | −1.174 | 0.133 | 0.276 | 2.05E−17 |
| TTC19 | 2.14E−21 | −0.677 | 0.22 | 0.319 | 2.44E−17 |
| P4HB | 2.49E−21 | −0.342 | 0.536 | 0.42 | 2.83E−17 |
| HEBP2 | 3.55E−21 | −0.931 | 0.274 | 0.402 | 4.04E−17 |
| MT-RNR1 | 3.66E−21 | −0.669 | 0.976 | 0.971 | 4.17E−17 |
| HMGB2 | 3.82E−21 | −0.795 | 0.227 | 0.423 | 4.35E−17 |
| CLTC | 4.20E−21 | −0.729 | 0.319 | 0.431 | 4.78E−17 |
| ETS1 | 4.77E−21 | −0.837 | 0.043 | 0.219 | 5.44E−17 |
| MRPL36 | 5.27E−21 | −0.918 | 0.223 | 0.304 | 6.00E−17 |
| CKS2 | 5.61E−21 | −0.940 | 0.203 | 0.314 | 6.40E−17 |
| TMEM165 | 5.76E−21 | −0.752 | 0.268 | 0.373 | 6.56E−17 |
| 7-Sep | 7.17E−21 | −0.778 | 0.339 | 0.436 | 8.17E−17 |
| SFT2D2 | 8.69E−21 | −0.851 | 0.081 | 0.265 | 9.91E−17 |
| LBR | 9.45E−21 | −1.000 | 0.119 | 0.306 | 1.08E−16 |
| PDIA6 | 9.90E−21 | −0.658 | 0.436 | 0.478 | 1.13E−16 |
| ISG15 | 1.19E−20 | −1.028 | 0.105 | 0.251 | 1.36E−16 |
| NDUFB7 | 1.35E−20 | −0.392 | 0.45 | 0.408 | 1.53E−16 |
| BCAP31 | 1.60E−20 | −0.614 | 0.311 | 0.275 | 1.82E−16 |
| CBR1 | 1.69E−20 | −0.789 | 0.122 | 0.228 | 1.92E−16 |
| CD9 | 2.45E−20 | −0.620 | 0.464 | 0.506 | 2.79E−16 |
| HSBP1 | 2.79E−20 | −0.651 | 0.468 | 0.461 | 3.18E−16 |
| ZNF706 | 2.94E−20 | −0.734 | 0.254 | 0.354 | 3.35E−16 |
| H3F3C | 2.97E−20 | −0.757 | 0.065 | 0.205 | 3.39E−16 |
| KPNA2 | 3.61E−20 | −0.949 | 0.101 | 0.24 | 4.11E−16 |
| RAB32 | 3.76E−20 | −0.735 | 0.019 | 0.164 | 4.28E−16 |
| HMGA1 | 3.96E−20 | −0.749 | 0.2 | 0.295 | 4.51E−16 |
| ARL6IP5 | 4.10E−20 | −0.607 | 0.341 | 0.306 | 4.67E−16 |
| CALU | 5.08E−20 | −0.616 | 0.334 | 0.358 | 5.79E−16 |
| ABL2 | 5.83E−20 | −0.781 | 0.036 | 0.193 | 6.65E−16 |
| PGRMC1 | 6.41E−20 | −0.869 | 0.228 | 0.354 | 7.30E−16 |
| HN1 | 9.36E−20 | −0.850 | 0.162 | 0.322 | 1.07E−15 |
| TAF1D | 9.44E−20 | −0.668 | 0.269 | 0.352 | 1.08E−15 |
| CHML | 1.51E−19 | −0.957 | 0.111 | 0.25 | 1.72E−15 |
| CCT3 | 1.56E−19 | −0.669 | 0.377 | 0.494 | 1.78E−15 |
| BIRC2 | 1.60E−19 | −0.853 | 0.128 | 0.23 | 1.82E−15 |
| RCN1 | 1.72E−19 | −0.871 | 0.146 | 0.257 | 1.96E−15 |
| MKI67 | 1.87E−19 | −0.999 | 0.098 | 0.26 | 2.13E−15 |
| USP1 | 1.89E−19 | −0.738 | 0.314 | 0.402 | 2.15E−15 |
| CPM | 2.18E−19 | −0.743 | 0.01 | 0.129 | 2.48E−15 |
| SFRP1 | 2.48E−19 | −0.862 | 0.006 | 0.114 | 2.83E−15 |
| RMRP | 2.76E−19 | −0.912 | 0.179 | 0.241 | 3.15E−15 |
| EIF4G1 | 4.07E−19 | −0.633 | 0.293 | 0.327 | 4.64E−15 |
| MT-ND4L | 4.16E−19 | −0.773 | 0.558 | 0.544 | 4.74E−15 |
| PARP1 | 4.23E−19 | −0.808 | 0.236 | 0.38 | 4.82E−15 |
| MAGOHB | 4.69E−19 | −0.849 | 0.095 | 0.224 | 5.34E−15 |
| ASPM | 5.06E−19 | −1.142 | 0.059 | 0.205 | 5.76E−15 |
| ART3 | 6.18E−19 | −0.812 | 0.006 | 0.118 | 7.04E−15 |
| SGCB | 6.39E−19 | −0.709 | 0.025 | 0.151 | 7.28E−15 |
| TIMP2 | 7.05E−19 | −0.798 | 0.017 | 0.142 | 8.03E−15 |
| SEC62 | 9.74E−19 | −0.603 | 0.407 | 0.455 | 1.11E−14 |
| MAP7D3 | 1.05E−18 | −0.649 | 0.006 | 0.121 | 1.20E−14 |
| SSR2 | 1.16E−18 | −0.474 | 0.379 | 0.428 | 1.33E−14 |
| PRDX4 | 1.20E−18 | −0.726 | 0.25 | 0.282 | 1.37E−14 |
| HSP90AA1 | 1.35E−18 | −0.291 | 0.906 | 0.912 | 1.54E−14 |
| SERF2 | 1.44E−18 | −0.539 | 0.781 | 0.766 | 1.64E−14 |
| PSMD11 | 1.51E−18 | −0.824 | 0.095 | 0.253 | 1.72E−14 |
| SOAT1 | 2.12E−18 | −0.813 | 0.046 | 0.189 | 2.42E−14 |
| PRDX6 | 2.16E−18 | −0.766 | 0.231 | 0.327 | 2.46E−14 |
| RAP2A | 2.31E−18 | −0.769 | 0.022 | 0.161 | 2.63E−14 |
| AIF1L | 2.31E−18 | −0.560 | 0.132 | 0.123 | 2.63E−14 |
| XIST | 2.58E−18 | −0.880 | 0.054 | 0.213 | 2.94E−14 |
| SLC29A1 | 2.66E−18 | −0.635 | 0.014 | 0.121 | 3.03E−14 |
| SKA2 | 2.82E−18 | −0.826 | 0.089 | 0.247 | 3.22E−14 |
| DCAF13 | 3.05E−18 | −0.849 | 0.127 | 0.273 | 3.48E−14 |
| SLC25A6 | 3.64E−18 | −0.613 | 0.398 | 0.423 | 4.15E−14 |
| SERBP1 | 3.66E−18 | −0.374 | 0.498 | 0.485 | 4.17E−14 |
| MT-CO3 | 3.85E−18 | −0.577 | 0.899 | 0.887 | 4.39E−14 |
| LSM5 | 4.39E−18 | −0.807 | 0.235 | 0.348 | 5.00E−14 |
| DNAJB11 | 4.55E−18 | −0.667 | 0.239 | 0.323 | 5.19E−14 |
| CYTH3 | 4.95E−18 | −0.608 | 0.008 | 0.111 | 5.64E−14 |
| KLF6 | 6.25E−18 | −0.693 | 0.479 | 0.539 | 7.12E−14 |
| TPR | 6.44E−18 | −0.513 | 0.361 | 0.487 | 7.34E−14 |
| RPS3 | 6.83E−18 | −0.453 | 0.83 | 0.797 | 7.78E−14 |
| QPCT | 6.98E−18 | −0.588 | 0.006 | 0.117 | 7.95E−14 |
| KRT5 | 7.33E−18 | −0.569 | 0.003 | 0.102 | 8.36E−14 |
| UGP2 | 8.11E−18 | −0.542 | 0.306 | 0.281 | 9.24E−14 |
| TPX2 | 8.22E−18 | −0.916 | 0.103 | 0.249 | 9.37E−14 |
| SLC36A4 | 8.72E−18 | −0.739 | 0.029 | 0.165 | 9.94E−14 |
| TBL1XR1 | 9.85E−18 | −0.700 | 0.304 | 0.38 | 1.12E−13 |
| DIAPH2 | 9.96E−18 | −0.724 | 0.014 | 0.139 | 1.14E−13 |
| NDUFA11 | 1.11E−17 | −0.334 | 0.422 | 0.389 | 1.27E−13 |
| NDUFB2 | 1.26E−17 | −0.506 | 0.433 | 0.44 | 1.43E−13 |
| ECHDC1 | 1.32E−17 | −0.670 | 0.022 | 0.151 | 1.50E−13 |
| POLR2B | 1.41E−17 | −0.846 | 0.179 | 0.304 | 1.61E−13 |
| GPR180 | 1.69E−17 | −0.705 | 0.036 | 0.18 | 1.92E−13 |
| STRAP | 1.69E−17 | −0.662 | 0.255 | 0.297 | 1.92E−13 |
| MRPL51 | 1.73E−17 | −0.586 | 0.303 | 0.327 | 1.97E−13 |
| EIF3K | 1.86E−17 | −0.489 | 0.377 | 0.374 | 2.12E−13 |
| LIMCH1 | 2.16E−17 | −0.337 | 0.197 | 0.124 | 2.46E−13 |
| G3BP2 | 2.51E−17 | −0.643 | 0.277 | 0.367 | 2.86E−13 |
| MEF2A | 2.53E−17 | −0.809 | 0.163 | 0.316 | 2.89E−13 |
| YWHAQ | 2.68E−17 | −0.748 | 0.268 | 0.371 | 3.05E−13 |
| SIVA1 | 2.85E−17 | −0.692 | 0.279 | 0.33 | 3.24E−13 |
| CRYAB | 3.59E−17 | −0.817 | 0.074 | 0.123 | 4.09E−13 |
| C11orf58 | 3.64E−17 | −0.513 | 0.407 | 0.402 | 4.15E−13 |
| MYBL1 | 3.73E−17 | −0.687 | 0.014 | 0.136 | 4.26E−13 |
| UCHL5 | 3.84E−17 | −0.880 | 0.135 | 0.287 | 4.38E−13 |
| STK24 | 4.04E−17 | −0.785 | 0.092 | 0.211 | 4.61E−13 |
| COX8A | 4.24E−17 | −0.510 | 0.404 | 0.44 | 4.83E−13 |
| LGALS3 | 4.31E−17 | −0.458 | 0.428 | 0.428 | 4.91E−13 |
| BTG1 | 4.82E−17 | −0.700 | 0.328 | 0.344 | 5.49E−13 |
| MT-CO1 | 5.00E−17 | −0.732 | 0.88 | 0.852 | 5.70E−13 |
| ITGB1 | 5.69E−17 | −0.727 | 0.227 | 0.387 | 6.48E−13 |
| ANKRD10 | 5.98E−17 | −0.793 | 0.071 | 0.225 | 6.82E−13 |
| ENSA | 6.15E−17 | −0.587 | 0.304 | 0.363 | 7.01E−13 |
| ATP5B | 6.33E−17 | −0.602 | 0.307 | 0.348 | 7.22E−13 |
| SACS | 7.08E−17 | −0.493 | 0.005 | 0.104 | 8.07E−13 |
| TMBIM6 | 8.13E−17 | −0.458 | 0.577 | 0.526 | 9.27E−13 |
| EGFR | 8.87E−17 | −0.520 | 0.003 | 0.101 | 1.01E−12 |
| HSP90B1 | 9.37E−17 | −0.496 | 0.639 | 0.656 | 1.07E−12 |
| TCEB1 | 9.56E−17 | −0.609 | 0.255 | 0.325 | 1.09E−12 |
| PHB2 | 9.94E−17 | −0.480 | 0.26 | 0.266 | 1.13E−12 |
| PRKX | 1.30E−16 | −0.643 | 0.008 | 0.11 | 1.48E−12 |
| SNRPD3 | 1.31E−16 | −0.493 | 0.365 | 0.363 | 1.49E−12 |
| METTL12 | 1.39E−16 | −0.508 | 0.371 | 0.319 | 1.59E−12 |
| ARPC2 | 1.46E−16 | −0.548 | 0.444 | 0.434 | 1.66E−12 |
| ATP5I | 1.52E−16 | −0.287 | 0.483 | 0.421 | 1.73E−12 |
| CAP1 | 1.53E−16 | −0.515 | 0.395 | 0.389 | 1.75E−12 |
| SGK1 | 1.60E−16 | −0.945 | 0.055 | 0.206 | 1.82E−12 |
| TGFBR1 | 1.66E−16 | −0.834 | 0.152 | 0.249 | 1.89E−12 |
| CAPG | 1.90E−16 | −0.767 | 0.128 | 0.178 | 2.17E−12 |
| ACTR2 | 1.97E−16 | −0.347 | 0.42 | 0.323 | 2.25E−12 |
| SF3B5 | 2.05E−16 | −0.582 | 0.279 | 0.325 | 2.34E−12 |
| MRPL33 | 2.12E−16 | −0.497 | 0.395 | 0.389 | 2.42E−12 |
| ARGLU1 | 2.68E−16 | −0.694 | 0.303 | 0.449 | 3.05E−12 |
| COTL1 | 2.77E−16 | −0.810 | 0.109 | 0.262 | 3.16E−12 |
| POLR2K | 2.94E−16 | −0.357 | 0.341 | 0.282 | 3.35E−12 |
| EIF3E | 2.97E−16 | −0.337 | 0.558 | 0.515 | 3.39E−12 |
| ANXA2 | 3.08E−16 | −0.460 | 0.515 | 0.488 | 3.51E−12 |
| MT-ATP8 | 3.26E−16 | −0.686 | 0.509 | 0.501 | 3.72E−12 |
| DDX21 | 3.31E−16 | −0.593 | 0.371 | 0.439 | 3.77E−12 |
| SRP72 | 3.48E−16 | −0.628 | 0.298 | 0.355 | 3.97E−12 |
| FBL | 3.61E−16 | −0.382 | 0.391 | 0.358 | 4.12E−12 |
| SEC61B | 3.89E−16 | −0.676 | 0.3 | 0.367 | 4.44E−12 |
| STRA13 | 4.07E−16 | −0.375 | 0.314 | 0.247 | 4.63E−12 |
| POGK | 4.31E−16 | −0.728 | 0.084 | 0.192 | 4.91E−12 |
| PLP2 | 4.59E−16 | −0.745 | 0.133 | 0.247 | 5.23E−12 |
| HSBP1L1 | 5.76E−16 | −0.428 | 0.12 | 0.088 | 6.56E−12 |
| CCT6A | 5.78E−16 | −0.461 | 0.437 | 0.463 | 6.59E−12 |
| TPM4 | 5.88E−16 | −0.309 | 0.605 | 0.611 | 6.70E−12 |
| TROVE2 | 7.02E−16 | −0.662 | 0.231 | 0.36 | 8.00E−12 |
| ARL6IP1 | 7.20E−16 | −0.364 | 0.429 | 0.329 | 8.21E−12 |
| TMEM183A | 7.61E−16 | −0.639 | 0.249 | 0.254 | 8.68E−12 |
| WDR34 | 7.76E−16 | −0.692 | 0.136 | 0.197 | 8.85E−12 |
| TM9SF3 | 7.87E−16 | −0.540 | 0.32 | 0.348 | 8.97E−12 |
| NOP56 | 8.33E−16 | −0.461 | 0.341 | 0.339 | 9.49E−12 |
| RPL23 | 8.45E−16 | −0.312 | 0.743 | 0.658 | 9.64E−12 |
| HOMER3 | 8.75E−16 | −0.604 | 0.014 | 0.12 | 9.98E−12 |
| PHGDH | 9.29E−16 | −0.674 | 0.174 | 0.225 | 1.06E−11 |
| DDAH2 | 9.51E−16 | −0.705 | 0.217 | 0.294 | 1.08E−11 |
| PHACTR2 | 1.14E−15 | −0.848 | 0.111 | 0.24 | 1.29E−11 |
| RNF181 | 1.27E−15 | −0.374 | 0.247 | 0.178 | 1.45E−11 |
| RASAL2 | 1.33E−15 | −0.577 | 0.014 | 0.126 | 1.51E−11 |
| CFL1 | 1.37E−15 | −0.414 | 0.585 | 0.563 | 1.56E−11 |
| MLF2 | 1.43E−15 | −0.600 | 0.257 | 0.291 | 1.63E−11 |
| PGM2L1 | 1.49E−15 | −0.787 | 0.032 | 0.149 | 1.70E−11 |
| PTBP3 | 1.57E−15 | −0.563 | 0.271 | 0.269 | 1.78E−11 |
| SON | 1.67E−15 | −0.503 | 0.404 | 0.439 | 1.91E−11 |
| CDKN1B | 1.86E−15 | −0.717 | 0.141 | 0.208 | 2.12E−11 |
| MPZL1 | 2.07E−15 | −0.735 | 0.211 | 0.294 | 2.36E−11 |
| SLC43A3 | 2.08E−15 | −0.628 | 0.011 | 0.115 | 2.37E−11 |
| NCAM1 | 2.22E−15 | −0.571 | 0.013 | 0.115 | 2.54E−11 |
| SVIP | 2.52E−15 | −0.807 | 0.254 | 0.37 | 2.87E−11 |
| C6orf62 | 3.39E−15 | −0.591 | 0.326 | 0.349 | 3.86E−11 |
| COX4I1 | 3.39E−15 | −0.392 | 0.593 | 0.513 | 3.87E−11 |
| TMEM176B | 3.49E−15 | −0.843 | 0.016 | 0.102 | 3.98E−11 |
| UQCRH | 3.90E−15 | −0.431 | 0.539 | 0.51 | 4.44E−11 |
| MET | 4.03E−15 | −0.654 | 0.01 | 0.107 | 4.59E−11 |
| ST14 | 4.04E−15 | −0.674 | 0.057 | 0.164 | 4.60E−11 |
| SNRPB | 4.36E−15 | −0.500 | 0.326 | 0.339 | 4.97E−11 |
| NANOS1 | 5.13E−15 | −0.658 | 0.081 | 0.129 | 5.85E−11 |
| ZNF43 | 5.20E−15 | −0.699 | 0.043 | 0.133 | 5.92E−11 |
| SMS | 5.62E−15 | −0.261 | 0.244 | 0.165 | 6.40E−11 |
| RBP7 | 6.26E−15 | −0.619 | 0.051 | 0.107 | 7.14E−11 |
| SCAF11 | 6.49E−15 | −0.624 | 0.347 | 0.399 | 7.40E−11 |
| CCDC82 | 6.52E−15 | −0.751 | 0.025 | 0.148 | 7.44E−11 |
| NUDCD1 | 6.77E−15 | −0.686 | 0.198 | 0.284 | 7.71E−11 |
| CD99 | 7.16E−15 | −0.674 | 0.143 | 0.18 | 8.16E−11 |
| ARPC5L | 7.21E−15 | −0.554 | 0.233 | 0.221 | 8.22E−11 |
| CCNE2 | 7.36E−15 | −0.780 | 0.128 | 0.24 | 8.39E−11 |
| H2AFV | 9.13E−15 | −0.628 | 0.241 | 0.338 | 1.04E−10 |
| RNA28S5 | 9.51E−15 | −0.500 | 0.842 | 0.848 | 1.08E−10 |
| COX6A1 | 1.00E−14 | −0.498 | 0.558 | 0.576 | 1.14E−10 |
| PTMS | 1.24E−14 | −0.703 | 0.284 | 0.335 | 1.41E−10 |
| NFE2L1 | 1.25E−14 | −0.617 | 0.158 | 0.192 | 1.43E−10 |
| POMP | 1.49E−14 | −0.731 | 0.282 | 0.386 | 1.70E−10 |
| MED17 | 1.53E−14 | −0.615 | 0.051 | 0.14 | 1.75E−10 |
| PSMC3 | 1.56E−14 | −0.698 | 0.246 | 0.327 | 1.78E−10 |
| MRPS12 | 1.57E−14 | −0.582 | 0.162 | 0.241 | 1.79E−10 |
| EMD | 1.60E−14 | −0.471 | 0.168 | 0.156 | 1.83E−10 |
| CTHRC1 | 1.62E−14 | −0.493 | 0.016 | 0.126 | 1.85E−10 |
| ECT2 | 1.80E−14 | −0.860 | 0.09 | 0.2 | 2.06E−10 |
| SESN3 | 1.84E−14 | −0.681 | 0.019 | 0.132 | 2.09E−10 |
| EIF4G2 | 2.35E−14 | −0.480 | 0.431 | 0.456 | 2.67E−10 |
| OAT | 2.41E−14 | −0.509 | 0.336 | 0.316 | 2.75E−10 |
| RPSAP58 | 2.53E−14 | −0.567 | 0.095 | 0.158 | 2.88E−10 |
| DUSP18 | 2.63E−14 | −0.592 | 0.073 | 0.2 | 3.00E−10 |
| RGS10 | 2.78E−14 | −0.772 | 0.189 | 0.287 | 3.17E−10 |
| RALA | 2.92E−14 | −0.788 | 0.147 | 0.266 | 3.33E−10 |
| RNF213 | 3.21E−14 | −0.509 | 0.279 | 0.265 | 3.66E−10 |
| POLR2L | 3.22E−14 | −0.489 | 0.352 | 0.338 | 3.67E−10 |
| HNRNPU | 3.55E−14 | −0.446 | 0.539 | 0.561 | 4.04E−10 |
| YWHAG | 3.56E−14 | −0.504 | 0.284 | 0.303 | 4.06E−10 |
| SLC35F2 | 4.13E−14 | −0.699 | 0.04 | 0.159 | 4.71E−10 |
| CKAP2 | 4.50E−14 | −0.781 | 0.046 | 0.158 | 5.13E−10 |
| LAMP1 | 5.00E−14 | −0.681 | 0.25 | 0.314 | 5.70E−10 |
| DDX5 | 5.00E−14 | −0.347 | 0.648 | 0.605 | 5.70E−10 |
| SLC25A39 | 5.20E−14 | −0.660 | 0.155 | 0.218 | 5.93E−10 |
| DNAJC3 | 5.23E−14 | −0.717 | 0.163 | 0.25 | 5.96E−10 |
| ATP6V0B | 5.24E−14 | −0.536 | 0.295 | 0.327 | 5.97E−10 |
| PRMT1 | 5.51E−14 | −0.585 | 0.203 | 0.279 | 6.28E−10 |
| SLC25A3 | 5.83E−14 | −0.495 | 0.301 | 0.308 | 6.65E−10 |
| RCAN1 | 6.07E−14 | −0.757 | 0.068 | 0.184 | 6.92E−10 |
| USMG5 | 6.12E−14 | −0.311 | 0.518 | 0.449 | 6.98E−10 |
| SKP1 | 6.33E−14 | −0.407 | 0.504 | 0.475 | 7.21E−10 |
| SMARCA1 | 6.67E−14 | −0.337 | 0.117 | 0.086 | 7.60E−10 |
| TWF1 | 7.69E−14 | −0.713 | 0.184 | 0.281 | 8.76E−10 |
| VPS28 | 7.75E−14 | −0.297 | 0.241 | 0.171 | 8.83E−10 |
| PDCD6 | 7.82E−14 | −0.604 | 0.279 | 0.3 | 8.92E−10 |
| PLEKHO1 | 7.86E−14 | −0.485 | 0.013 | 0.114 | 8.96E−10 |
| HIST1H2BK | 7.98E−14 | −0.430 | 0.212 | 0.171 | 9.09E−10 |
| PPAT | 8.17E−14 | −0.747 | 0.068 | 0.197 | 9.31E−10 |
| MIR4458HG | 8.61E−14 | −0.469 | 0.189 | 0.164 | 9.81E−10 |
| CTSB | 9.82E−14 | −0.755 | 0.155 | 0.31 | 1.12E−09 |
| CHCHD3 | 9.90E−14 | −0.611 | 0.157 | 0.228 | 1.13E−09 |
| EIF1AX | 1.04E−13 | −0.472 | 0.276 | 0.25 | 1.18E−09 |
| MBOAT1 | 1.05E−13 | −0.608 | 0.029 | 0.129 | 1.19E−09 |
| COX6B1 | 1.08E−13 | −0.288 | 0.567 | 0.51 | 1.24E−09 |
| LAMC1 | 1.24E−13 | −0.540 | 0.014 | 0.11 | 1.41E−09 |
| LSM4 | 1.25E−13 | −0.611 | 0.236 | 0.301 | 1.42E−09 |
| ITM2B | 1.29E−13 | −0.612 | 0.282 | 0.349 | 1.47E−09 |
| SPIN1 | 1.29E−13 | −0.679 | 0.117 | 0.197 | 1.47E−09 |
| PRRC2C | 1.40E−13 | −0.288 | 0.542 | 0.538 | 1.60E−09 |
| TIMM17A | 1.51E−13 | −0.762 | 0.133 | 0.238 | 1.73E−09 |
| PTGS2 | 1.58E−13 | −1.024 | 0.016 | 0.11 | 1.81E−09 |
| LY6E | 1.60E−13 | −0.597 | 0.3 | 0.304 | 1.82E−09 |
| HIPK3 | 1.70E−13 | −0.725 | 0.082 | 0.181 | 1.94E−09 |
| MRPL34 | 1.76E−13 | −0.663 | 0.185 | 0.259 | 2.01E−09 |
| HDAC2 | 1.88E−13 | −0.525 | 0.247 | 0.385 | 2.14E−09 |
| PSMD2 | 1.98E−13 | −0.472 | 0.304 | 0.295 | 2.25E−09 |
| RAB14 | 2.05E−13 | −0.322 | 0.296 | 0.231 | 2.34E−09 |
| ATP6AP1 | 2.06E−13 | −0.463 | 0.184 | 0.177 | 2.34E−09 |
| CCDC47 | 2.19E−13 | −0.552 | 0.355 | 0.417 | 2.50E−09 |
| SYNE2 | 2.21E−13 | −0.410 | 0.425 | 0.525 | 2.52E−09 |
| ATP1B1 | 2.42E−13 | −0.666 | 0.23 | 0.325 | 2.76E−09 |
| DYNLL1 | 2.45E−13 | −0.463 | 0.385 | 0.431 | 2.79E−09 |
| U2AF1 | 2.46E−13 | −0.633 | 0.266 | 0.342 | 2.81E−09 |
| SF3B2 | 2.56E−13 | −0.516 | 0.257 | 0.238 | 2.91E−09 |
| RAC1 | 2.61E−13 | −0.398 | 0.317 | 0.31 | 2.97E−09 |
| WBP2 | 2.73E−13 | −0.262 | 0.146 | 0.098 | 3.11E−09 |
| CCNB1 | 2.91E−13 | −0.845 | 0.063 | 0.187 | 3.32E−09 |
| AURKAIP1 | 3.02E−13 | −0.689 | 0.185 | 0.244 | 3.44E−09 |
| IDI1 | 3.47E−13 | −0.791 | 0.212 | 0.329 | 3.96E−09 |
| WHSC1 | 3.48E−13 | −0.719 | 0.092 | 0.184 | 3.96E−09 |
| NDUFA6 | 3.56E−13 | −0.574 | 0.42 | 0.396 | 4.06E−09 |
| HNRNPM | 3.73E−13 | −0.523 | 0.415 | 0.442 | 4.25E−09 |
| GMNN | 3.75E−13 | −0.692 | 0.136 | 0.238 | 4.27E−09 |
| MT-ND4 | 4.05E−13 | −0.595 | 0.897 | 0.877 | 4.61E−09 |
| EPB41L2 | 4.21E−13 | −0.640 | 0.038 | 0.164 | 4.80E−09 |
| NME1 | 4.61E−13 | −0.464 | 0.353 | 0.348 | 5.25E−09 |
| GGH | 4.71E−13 | −0.655 | 0.079 | 0.18 | 5.37E−09 |
| CPQ | 4.98E−13 | −0.688 | 0.054 | 0.167 | 5.68E−09 |
| PCBP2 | 5.24E−13 | −0.505 | 0.322 | 0.354 | 5.98E−09 |
| FARP1 | 5.42E−13 | −0.765 | 0.13 | 0.219 | 6.17E−09 |
| RHOA | 5.47E−13 | −0.383 | 0.417 | 0.412 | 6.23E−09 |
| PSMB4 | 5.64E−13 | −0.649 | 0.217 | 0.316 | 6.43E−09 |
| KIF5B | 5.95E−13 | −0.508 | 0.315 | 0.364 | 6.79E−09 |
| TOP2A | 6.30E−13 | −0.749 | 0.149 | 0.292 | 7.18E−09 |
| NDUFA3 | 6.47E−13 | −0.486 | 0.328 | 0.317 | 7.38E−09 |
| DDX39A | 6.66E−13 | −0.613 | 0.124 | 0.212 | 7.59E−09 |
| EDARADD | 6.76E−13 | −0.765 | 0.128 | 0.256 | 7.71E−09 |
| PET100 | 6.85E−13 | −0.385 | 0.323 | 0.317 | 7.80E−09 |
| PPIA | 7.28E−13 | −0.381 | 0.406 | 0.355 | 8.29E−09 |
| H3F3A | 7.41E−13 | −0.674 | 0.051 | 0.11 | 8.44E−09 |
| E2F3 | 7.96E−13 | −0.661 | 0.1 | 0.227 | 9.08E−09 |
| LTBR | 8.31E−13 | −0.746 | 0.086 | 0.2 | 9.47E−09 |
| ATP5G3 | 8.63E−13 | −0.385 | 0.39 | 0.387 | 9.84E−09 |
| NASP | 9.62E−13 | −0.636 | 0.222 | 0.322 | 1.10E−08 |
| HNRNPF | 1.00E−12 | −0.511 | 0.319 | 0.349 | 1.14E−08 |
| MRPL14 | 1.10E−12 | −0.511 | 0.254 | 0.275 | 1.25E−08 |
| CCNG2 | 1.12E−12 | −0.733 | 0.189 | 0.26 | 1.27E−08 |
| TFRC | 1.17E−12 | −0.566 | 0.281 | 0.282 | 1.33E−08 |
| MRPS35 | 1.17E−12 | −0.382 | 0.388 | 0.374 | 1.33E−08 |
| USO1 | 1.23E−12 | −0.489 | 0.265 | 0.289 | 1.40E−08 |
| EIF2AK2 | 1.24E−12 | −0.565 | 0.249 | 0.243 | 1.41E−08 |
| GATAD2A | 1.26E−12 | −0.764 | 0.089 | 0.194 | 1.44E−08 |
| TUBGCP3 | 1.33E−12 | −0.720 | 0.055 | 0.173 | 1.51E−08 |
| RCN2 | 1.33E−12 | −0.446 | 0.231 | 0.234 | 1.52E−08 |
| COPB2 | 1.36E−12 | −0.258 | 0.287 | 0.205 | 1.55E−08 |
| PRMT2 | 1.37E−12 | −0.516 | 0.304 | 0.354 | 1.56E−08 |
| PSMC5 | 1.39E−12 | −0.524 | 0.238 | 0.266 | 1.58E−08 |
| MRPL1 | 1.48E−12 | −0.695 | 0.139 | 0.273 | 1.68E−08 |
| HELLS | 1.50E−12 | −0.757 | 0.068 | 0.19 | 1.71E−08 |
| TMEM64 | 1.54E−12 | −0.506 | 0.014 | 0.11 | 1.76E−08 |
| MIR4435-1HG | 2.03E−12 | −0.573 | 0.025 | 0.129 | 2.31E−08 |
| TSEN15 | 2.09E−12 | −0.597 | 0.071 | 0.113 | 2.38E−08 |
| DCK | 2.12E−12 | −0.587 | 0.032 | 0.126 | 2.42E−08 |
| MOB1B | 2.14E−12 | −0.668 | 0.049 | 0.164 | 2.44E−08 |
| CAPS | 2.26E−12 | −0.673 | 0.049 | 0.135 | 2.58E−08 |
| HLA-C | 2.32E−12 | −0.474 | 0.406 | 0.383 | 2.65E−08 |
| PRDX5 | 2.43E−12 | −0.393 | 0.273 | 0.234 | 2.77E−08 |
| NENF | 2.44E−12 | −0.504 | 0.179 | 0.174 | 2.78E−08 |
| NDUFB11 | 2.44E−12 | −0.553 | 0.255 | 0.275 | 2.78E−08 |
| TAF15 | 2.50E−12 | −0.631 | 0.12 | 0.213 | 2.85E−08 |
| HIST1H1B | 2.75E−12 | −0.760 | 0.035 | 0.148 | 3.13E−08 |
| UBE2D3 | 2.78E−12 | −0.265 | 0.387 | 0.31 | 3.17E−08 |
| TOR3A | 2.88E−12 | −0.561 | 0.038 | 0.14 | 3.28E−08 |
| SUZ12 | 2.98E−12 | −0.552 | 0.078 | 0.211 | 3.40E−08 |
| SCCPDH | 3.05E−12 | −0.659 | 0.055 | 0.126 | 3.48E−08 |
| FKBP3 | 3.06E−12 | −0.426 | 0.344 | 0.345 | 3.48E−08 |
| SLC39A1 | 3.12E−12 | −0.605 | 0.092 | 0.197 | 3.56E−08 |
| DNM1L | 3.33E−12 | −0.762 | 0.136 | 0.25 | 3.79E−08 |
| RPL38 | 3.64E−12 | −0.302 | 0.914 | 0.868 | 4.15E−08 |
| LCP1 | 4.01E−12 | −0.483 | 0.109 | 0.256 | 4.57E−08 |
| PTGES3 | 4.51E−12 | −0.464 | 0.393 | 0.409 | 5.14E−08 |
| FBXO32 | 4.91E−12 | −0.822 | 0.143 | 0.208 | 5.59E−08 |
| NF1 | 5.10E−12 | −0.567 | 0.038 | 0.143 | 5.82E−08 |
| NONO | 5.23E−12 | −0.470 | 0.303 | 0.333 | 5.96E−08 |
| RPL35 | 5.89E−12 | −0.412 | 0.929 | 0.936 | 6.71E−08 |
| CUL4A | 6.02E−12 | −0.691 | 0.097 | 0.23 | 6.86E−08 |
| B4GALT1 | 6.16E−12 | −0.741 | 0.146 | 0.227 | 7.02E−08 |
| EIF1 | 6.66E−12 | −0.379 | 0.628 | 0.677 | 7.59E−08 |
| HTATIP2 | 6.86E−12 | −0.692 | 0.109 | 0.152 | 7.82E−08 |
| TMEM30A | 7.05E−12 | −0.649 | 0.217 | 0.317 | 8.03E−08 |
| C21orf91 | 7.06E−12 | −0.526 | 0.021 | 0.12 | 8.05E−08 |
| CHEK1 | 7.18E−12 | −0.536 | 0.03 | 0.124 | 8.19E−08 |
| SLC3A2 | 7.54E−12 | −0.476 | 0.238 | 0.243 | 8.59E−08 |
| PSMD12 | 7.57E−12 | −0.693 | 0.174 | 0.304 | 8.62E−08 |
| COLGALT1 | 7.58E−12 | −0.572 | 0.1 | 0.154 | 8.64E−08 |
| LMNB1 | 7.70E−12 | −0.616 | 0.043 | 0.158 | 8.78E−08 |
| BRD4 | 7.84E−12 | −0.542 | 0.257 | 0.282 | 8.93E−08 |
| CRK | 8.50E−12 | −0.642 | 0.092 | 0.19 | 9.69E−08 |
| IQCG | 8.52E−12 | −0.489 | 0.065 | 0.114 | 9.71E−08 |
| CD82 | 8.92E−12 | −0.284 | 0.228 | 0.173 | 1.02E−07 |
| CHERP | 9.41E−12 | −0.574 | 0.049 | 0.126 | 1.07E−07 |
| GRN | 9.46E−12 | −0.757 | 0.055 | 0.165 | 1.08E−07 |
| HM13 | 9.50E−12 | −0.505 | 0.197 | 0.224 | 1.08E−07 |
| CAPZB | 9.82E−12 | −0.316 | 0.298 | 0.228 | 1.12E−07 |
| GNB1 | 9.96E−12 | −0.561 | 0.258 | 0.294 | 1.14E−07 |
| SNRPF | 1.03E−11 | −0.558 | 0.2 | 0.249 | 1.17E−07 |
| TOR1AIP1 | 1.20E−11 | −0.647 | 0.081 | 0.219 | 1.36E−07 |
| COX14 | 1.23E−11 | −0.348 | 0.201 | 0.171 | 1.41E−07 |
| JTB | 1.26E−11 | −0.534 | 0.301 | 0.349 | 1.43E−07 |
| MRPL20 | 1.28E−11 | −0.505 | 0.252 | 0.327 | 1.46E−07 |
| NUP54 | 1.28E−11 | −0.623 | 0.073 | 0.189 | 1.46E−07 |
| PHB | 1.30E−11 | −0.575 | 0.201 | 0.288 | 1.48E−07 |
| DESI2 | 1.44E−11 | −0.720 | 0.1 | 0.19 | 1.64E−07 |
| MYEOV2 | 1.45E−11 | −0.427 | 0.433 | 0.389 | 1.66E−07 |
| TEX30 | 1.53E−11 | −0.571 | 0.059 | 0.18 | 1.75E−07 |
| TMEM14A | 1.59E−11 | −0.702 | 0.059 | 0.121 | 1.82E−07 |
| TOMM20 | 1.68E−11 | −0.325 | 0.487 | 0.465 | 1.92E−07 |
| CDC42EP1 | 1.73E−11 | −0.297 | 0.119 | 0.095 | 1.97E−07 |
| CYBA | 1.85E−11 | −0.802 | 0.052 | 0.136 | 2.11E−07 |
| LMAN2 | 2.01E−11 | −0.354 | 0.281 | 0.235 | 2.29E−07 |
| AMD1 | 2.13E−11 | −0.647 | 0.236 | 0.354 | 2.43E−07 |
| ANXA7 | 2.25E−11 | −0.315 | 0.23 | 0.17 | 2.57E−07 |
| IRS2 | 2.38E−11 | −0.643 | 0.074 | 0.211 | 2.72E−07 |
| ATP11A | 2.44E−11 | −0.603 | 0.038 | 0.136 | 2.78E−07 |
| UBE2E1 | 2.78E−11 | −0.456 | 0.185 | 0.189 | 3.17E−07 |
| ECHS1 | 2.82E−11 | −0.537 | 0.179 | 0.266 | 3.22E−07 |
| GLIPR2 | 3.07E−11 | −0.483 | 0.016 | 0.107 | 3.49E−07 |
| GTF3A | 3.07E−11 | −0.732 | 0.198 | 0.291 | 3.50E−07 |
| PTS | 3.09E−11 | −0.611 | 0.097 | 0.203 | 3.53E−07 |
| ERH | 3.20E−11 | −0.310 | 0.417 | 0.373 | 3.65E−07 |
| EZR | 3.38E−11 | −0.332 | 0.423 | 0.363 | 3.85E−07 |
| FDPS | 3.41E−11 | −0.696 | 0.22 | 0.292 | 3.89E−07 |
| COPA | 3.54E−11 | −0.563 | 0.182 | 0.251 | 4.04E−07 |
| FAM50A | 3.56E−11 | −0.293 | 0.155 | 0.124 | 4.06E−07 |
| TRPS1 | 3.61E−11 | −0.368 | 0.536 | 0.488 | 4.11E−07 |
| MTCH2 | 3.71E−11 | −0.507 | 0.149 | 0.181 | 4.22E−07 |
| DIAPH3 | 3.88E−11 | −0.682 | 0.025 | 0.114 | 4.43E−07 |
| MED21 | 3.94E−11 | −0.551 | 0.046 | 0.135 | 4.50E−07 |
| CASP8AP2 | 4.00E−11 | −0.590 | 0.095 | 0.222 | 4.56E−07 |
| SUMO3 | 4.04E−11 | −0.413 | 0.22 | 0.215 | 4.60E−07 |
| CDK6 | 4.07E−11 | −0.614 | 0.049 | 0.165 | 4.64E−07 |
| MYH9 | 4.09E−11 | −0.524 | 0.322 | 0.427 | 4.66E−07 |
| COX5B | 4.14E−11 | −0.471 | 0.455 | 0.49 | 4.72E−07 |
| C11orf31 | 4.34E−11 | −0.369 | 0.298 | 0.282 | 4.95E−07 |
| HINT1 | 4.48E−11 | −0.254 | 0.559 | 0.513 | 5.10E−07 |
| BMP2K | 4.66E−11 | −0.569 | 0.036 | 0.135 | 5.32E−07 |
| TOR1AIP2 | 4.79E−11 | −0.627 | 0.182 | 0.275 | 5.46E−07 |
| RP11-58E21.4 | 4.83E−11 | −0.521 | 0.07 | 0.148 | 5.50E−07 |
| MUC16 | 4.90E−11 | −0.662 | 0.051 | 0.13 | 5.58E−07 |
| IARS2 | 5.05E−11 | −0.578 | 0.239 | 0.262 | 5.75E−07 |
| CST3 | 5.26E−11 | −0.264 | 0.266 | 0.203 | 5.99E−07 |
| PPIB | 5.51E−11 | −0.461 | 0.357 | 0.351 | 6.28E−07 |
| CCDC109B | 5.58E−11 | −0.661 | 0.059 | 0.137 | 6.36E−07 |
| SNRPG | 5.62E−11 | −0.468 | 0.32 | 0.363 | 6.40E−07 |
| PTP4A1 | 5.91E−11 | −0.544 | 0.271 | 0.33 | 6.74E−07 |
| LAMP2 | 6.73E−11 | −0.514 | 0.277 | 0.288 | 7.67E−07 |
| ACBD6 | 6.79E−11 | −0.567 | 0.133 | 0.161 | 7.74E−07 |
| SMC5 | 7.20E−11 | −0.662 | 0.09 | 0.203 | 8.21E−07 |
| STK38L | 7.68E−11 | −0.694 | 0.117 | 0.193 | 8.76E−07 |
| HSPA5 | 8.37E−11 | −0.455 | 0.537 | 0.534 | 9.54E−07 |
| EIF3A | 8.52E−11 | −0.270 | 0.377 | 0.349 | 9.71E−07 |
| PTAR1 | 8.73E−11 | −0.516 | 0.021 | 0.108 | 9.95E−07 |
| RARRES1 | 8.99E−11 | −0.759 | 0.254 | 0.303 | 1.02E−06 |
| LSM7 | 9.49E−11 | −0.463 | 0.338 | 0.36 | 1.08E−06 |
| ISYNA1 | 9.78E−11 | −0.350 | 0.063 | 0.102 | 1.11E−06 |
| CDC16 | 1.00E−10 | −0.705 | 0.138 | 0.224 | 1.14E−06 |
| STARD7 | 1.00E−10 | −0.572 | 0.193 | 0.259 | 1.15E−06 |
| MLF1IP | 1.01E−10 | −0.683 | 0.074 | 0.173 | 1.15E−06 |
| CARHSP1 | 1.11E−10 | −0.483 | 0.127 | 0.146 | 1.26E−06 |
| TMEM106C | 1.13E−10 | −0.656 | 0.132 | 0.228 | 1.29E−06 |
| EIF5A | 1.14E−10 | −0.487 | 0.279 | 0.291 | 1.30E−06 |
| TSPO | 1.15E−10 | −0.283 | 0.417 | 0.345 | 1.31E−06 |
| NBPF1 | 1.15E−10 | −0.503 | 0.1 | 0.14 | 1.32E−06 |
| CAT | 1.17E−10 | −0.565 | 0.063 | 0.118 | 1.33E−06 |
| PPHLN1 | 1.20E−10 | −0.602 | 0.116 | 0.192 | 1.37E−06 |
| DEGS1 | 1.29E−10 | −0.672 | 0.158 | 0.247 | 1.47E−06 |
| ABCF1 | 1.33E−10 | −0.428 | 0.246 | 0.24 | 1.51E−06 |
| GLS | 1.33E−10 | −0.670 | 0.082 | 0.193 | 1.52E−06 |
| G3BP1 | 1.34E−10 | −0.403 | 0.317 | 0.294 | 1.53E−06 |
| TUBGCP2 | 1.36E−10 | −0.473 | 0.139 | 0.126 | 1.55E−06 |
| SEPW1 | 1.43E−10 | −0.383 | 0.372 | 0.363 | 1.63E−06 |
| ARHGAP11A | 1.44E−10 | −0.372 | 0.033 | 0.124 | 1.65E−06 |
| PRPF40A | 1.52E−10 | −0.311 | 0.403 | 0.349 | 1.74E−06 |
| TRA2B | 1.54E−10 | −0.461 | 0.314 | 0.31 | 1.75E−06 |
| ARHGEF7 | 1.54E−10 | −0.611 | 0.062 | 0.146 | 1.76E−06 |
| TCEB3B | 1.65E−10 | −0.653 | 0.054 | 0.165 | 1.88E−06 |
| CAPNS1 | 1.65E−10 | −0.321 | 0.244 | 0.199 | 1.89E−06 |
| CREG1 | 1.71E−10 | −0.555 | 0.155 | 0.193 | 1.95E−06 |
| CREB5 | 1.74E−10 | −0.462 | 0.022 | 0.107 | 1.98E−06 |
| SDAD1 | 1.87E−10 | −0.581 | 0.138 | 0.199 | 2.13E−06 |
| C9orf16 | 1.88E−10 | −0.370 | 0.193 | 0.17 | 2.15E−06 |
| SAP18 | 1.93E−10 | −0.508 | 0.255 | 0.276 | 2.20E−06 |
| WDR45B | 1.96E−10 | −0.315 | 0.276 | 0.219 | 2.23E−06 |
| PCNP | 1.99E−10 | −0.615 | 0.222 | 0.31 | 2.27E−06 |
| HNRNPD | 2.10E−10 | −0.393 | 0.33 | 0.336 | 2.39E−06 |
| CHMP2B | 2.11E−10 | −0.274 | 0.273 | 0.212 | 2.41E−06 |
| RER1 | 2.16E−10 | −0.582 | 0.144 | 0.177 | 2.46E−06 |
| ELP5 | 2.18E−10 | −0.499 | 0.101 | 0.148 | 2.48E−06 |
| YY1 | 2.21E−10 | −0.287 | 0.384 | 0.364 | 2.52E−06 |
| BOD1 | 2.38E−10 | −0.275 | 0.146 | 0.108 | 2.71E−06 |
| GNAS | 2.54E−10 | −0.271 | 0.501 | 0.434 | 2.89E−06 |
| ARMCX3 | 2.61E−10 | −0.619 | 0.108 | 0.186 | 2.98E−06 |
| VMA21 | 2.64E−10 | −0.502 | 0.163 | 0.18 | 3.01E−06 |
| IQGAP1 | 2.67E−10 | −0.284 | 0.569 | 0.494 | 3.04E−06 |
| BEX4 | 2.79E−10 | −0.417 | 0.139 | 0.143 | 3.17E−06 |
| CADM1 | 2.80E−10 | −0.650 | 0.067 | 0.159 | 3.19E−06 |
| CHD7 | 2.88E−10 | −0.570 | 0.041 | 0.137 | 3.28E−06 |
| IRAK1 | 2.93E−10 | −0.626 | 0.09 | 0.196 | 3.34E−06 |
| CHCHD7 | 3.00E−10 | −0.571 | 0.06 | 0.175 | 3.42E−06 |
| DLAT | 3.18E−10 | −0.580 | 0.063 | 0.148 | 3.62E−06 |
| APOA1BP | 3.23E−10 | −0.485 | 0.157 | 0.192 | 3.68E−06 |
| VAMP7 | 3.33E−10 | −0.363 | 0.108 | 0.104 | 3.79E−06 |
| APH1A | 3.36E−10 | −0.508 | 0.219 | 0.235 | 3.83E−06 |
| TUBB6 | 3.44E−10 | −0.463 | 0.046 | 0.113 | 3.92E−06 |
| PFDN4 | 3.53E−10 | −0.603 | 0.274 | 0.326 | 4.02E−06 |
| IFT57 | 3.53E−10 | −0.686 | 0.154 | 0.203 | 4.03E−06 |
| MAML2 | 3.61E−10 | −0.444 | 0.017 | 0.102 | 4.11E−06 |
| AKR1A1 | 3.75E−10 | −0.489 | 0.166 | 0.173 | 4.27E−06 |
| PPP2CA | 3.76E−10 | −0.409 | 0.203 | 0.193 | 4.28E−06 |
| ZNF511 | 3.93E−10 | −0.529 | 0.078 | 0.137 | 4.48E−06 |
| KPNB1 | 4.31E−10 | −0.405 | 0.347 | 0.338 | 4.91E−06 |
| KRAS | 4.33E−10 | −0.613 | 0.133 | 0.196 | 4.94E−06 |
| ELOVL6 | 4.60E−10 | −0.655 | 0.06 | 0.175 | 5.25E−06 |
| TPP2 | 4.71E−10 | −0.558 | 0.087 | 0.2 | 5.37E−06 |
| NEK2 | 5.32E−10 | −0.556 | 0.029 | 0.118 | 6.06E−06 |
| MSN | 5.39E−10 | −0.639 | 0.046 | 0.155 | 6.15E−06 |
| VDAC1 | 5.55E−10 | −0.397 | 0.303 | 0.273 | 6.33E−06 |
| WWTR1 | 5.58E−10 | −0.594 | 0.071 | 0.193 | 6.36E−06 |
| C16orf13 | 5.68E−10 | −0.429 | 0.189 | 0.173 | 6.48E−06 |
| CHMP1A | 6.08E−10 | −0.423 | 0.127 | 0.123 | 6.93E−06 |
| KATNBL1 | 6.08E−10 | −0.350 | 0.157 | 0.126 | 6.93E−06 |
| ACAT2 | 6.11E−10 | −0.578 | 0.095 | 0.196 | 6.97E−06 |
| GHITM | 6.19E−10 | −0.447 | 0.282 | 0.262 | 7.06E−06 |
| VGLL4 | 6.24E−10 | −0.478 | 0.147 | 0.155 | 7.11E−06 |
| ITGA6 | 6.24E−10 | −0.624 | 0.084 | 0.178 | 7.12E−06 |
| MTMR2 | 6.30E−10 | −0.530 | 0.04 | 0.124 | 7.18E−06 |
| TAPBP | 6.56E−10 | −0.583 | 0.17 | 0.2 | 7.48E−06 |
| SUN1 | 6.71E−10 | −0.639 | 0.087 | 0.19 | 7.64E−06 |
| PCNA | 6.96E−10 | −0.622 | 0.149 | 0.206 | 7.93E−06 |
| MYC | 7.44E−10 | −0.642 | 0.287 | 0.339 | 8.48E−06 |
| SSB | 7.88E−10 | −0.260 | 0.361 | 0.354 | 8.98E−06 |
| CAPRIN1 | 7.97E−10 | −0.504 | 0.209 | 0.251 | 9.08E−06 |
| NOP16 | 8.19E−10 | −0.620 | 0.128 | 0.205 | 9.34E−06 |
| PALLD | 8.20E−10 | −0.618 | 0.089 | 0.177 | 9.34E−06 |
| MRPL39 | 8.23E−10 | −0.500 | 0.036 | 0.132 | 9.38E−06 |
| TMCO1 | 8.94E−10 | −0.461 | 0.311 | 0.382 | 1.02E−05 |
| NPC2 | 9.60E−10 | −0.552 | 0.247 | 0.313 | 1.09E−05 |
| PLSCR1 | 9.72E−10 | −0.731 | 0.105 | 0.219 | 1.11E−05 |
| STIP1 | 1.01E−09 | −0.491 | 0.211 | 0.213 | 1.15E−05 |
| BID | 1.08E−09 | −0.562 | 0.073 | 0.17 | 1.23E−05 |
| PRC1 | 1.12E−09 | −0.688 | 0.068 | 0.14 | 1.28E−05 |
| KIAA0020 | 1.14E−09 | −0.412 | 0.273 | 0.361 | 1.30E−05 |
| CIB1 | 1.25E−09 | −0.378 | 0.158 | 0.168 | 1.43E−05 |
| KLHL42 | 1.27E−09 | −0.527 | 0.092 | 0.165 | 1.44E−05 |
| MRPL24 | 1.30E−09 | −0.286 | 0.176 | 0.132 | 1.48E−05 |
| C6orf106 | 1.41E−09 | −0.399 | 0.128 | 0.113 | 1.60E−05 |
| CWC15 | 1.48E−09 | −0.469 | 0.273 | 0.285 | 1.69E−05 |
| MRFAP1 | 1.52E−09 | −0.414 | 0.249 | 0.232 | 1.74E−05 |
| PADI2 | 1.58E−09 | −0.485 | 0.132 | 0.146 | 1.80E−05 |
| RPL22L1 | 1.64E−09 | −0.400 | 0.271 | 0.247 | 1.87E−05 |
| NUP153 | 1.73E−09 | −0.637 | 0.089 | 0.17 | 1.97E−05 |
| POLR2H | 1.73E−09 | −0.457 | 0.192 | 0.263 | 1.98E−05 |
| POLR2I | 1.74E−09 | −0.417 | 0.235 | 0.249 | 1.98E−05 |
| DPYSL2 | 1.75E−09 | −0.592 | 0.117 | 0.205 | 1.99E−05 |
| ADAR | 1.75E−09 | −0.482 | 0.255 | 0.279 | 2.00E−05 |
| PSMB1 | 1.75E−09 | −0.429 | 0.306 | 0.319 | 2.00E−05 |
| UAP1 | 1.78E−09 | −0.484 | 0.176 | 0.205 | 2.03E−05 |
| PRNP | 1.78E−09 | −0.640 | 0.1 | 0.197 | 2.03E−05 |
| HNRNPH1 | 1.83E−09 | −0.499 | 0.36 | 0.405 | 2.09E−05 |
| ZNF430 | 1.84E−09 | −0.389 | 0.019 | 0.101 | 2.09E−05 |
| ANAPC11 | 1.88E−09 | −0.263 | 0.48 | 0.424 | 2.14E−05 |
| SSBP1 | 2.00E−09 | −0.447 | 0.349 | 0.373 | 2.28E−05 |
| ARCN1 | 2.13E−09 | −0.517 | 0.166 | 0.189 | 2.43E−05 |
| TMEM126B | 2.14E−09 | −0.591 | 0.111 | 0.189 | 2.44E−05 |
| DDX6 | 2.26E−09 | −0.532 | 0.195 | 0.231 | 2.57E−05 |
| SBDS | 2.30E−09 | −0.417 | 0.236 | 0.216 | 2.62E−05 |
| RIPK2 | 2.30E−09 | −0.549 | 0.092 | 0.148 | 2.63E−05 |
| DYNLT1 | 2.30E−09 | −0.362 | 0.25 | 0.215 | 2.63E−05 |
| ANAPC5 | 2.31E−09 | −0.330 | 0.241 | 0.211 | 2.63E−05 |
| DPM3 | 2.34E−09 | −0.450 | 0.228 | 0.241 | 2.67E−05 |
| TCEAL8 | 2.64E−09 | −0.491 | 0.258 | 0.265 | 3.00E−05 |
| QSER1 | 2.66E−09 | −0.309 | 0.247 | 0.209 | 3.03E−05 |
| CNIH1 | 2.73E−09 | −0.278 | 0.285 | 0.266 | 3.11E−05 |
| MRPS7 | 2.75E−09 | −0.402 | 0.222 | 0.219 | 3.13E−05 |
| UQCRQ | 2.83E−09 | −0.283 | 0.563 | 0.522 | 3.23E−05 |
| ERP44 | 2.83E−09 | −0.590 | 0.097 | 0.158 | 3.23E−05 |
| COMMD9 | 2.87E−09 | −0.570 | 0.041 | 0.113 | 3.27E−05 |
| VCL | 2.90E−09 | −0.403 | 0.258 | 0.244 | 3.31E−05 |
| CD52 | 2.90E−09 | −0.445 | 0.055 | 0.162 | 3.31E−05 |
| VBP1 | 2.99E−09 | −0.294 | 0.307 | 0.263 | 3.41E−05 |
| SNRPA1 | 3.06E−09 | −0.684 | 0.084 | 0.154 | 3.49E−05 |
| TMED2 | 3.09E−09 | −0.421 | 0.349 | 0.326 | 3.52E−05 |
| DLGAP5 | 3.09E−09 | −0.589 | 0.049 | 0.136 | 3.52E−05 |
| ACTR3 | 3.14E−09 | −0.446 | 0.281 | 0.32 | 3.58E−05 |
| TMED9 | 3.21E−09 | −0.307 | 0.322 | 0.284 | 3.66E−05 |
| TATDN1 | 3.22E−09 | −0.588 | 0.192 | 0.228 | 3.67E−05 |
| 15-Sep | 3.25E−09 | −0.409 | 0.263 | 0.278 | 3.71E−05 |
| KHDRBS1 | 3.40E−09 | −0.351 | 0.306 | 0.278 | 3.87E−05 |
| ANXA5 | 3.47E−09 | −0.288 | 0.379 | 0.329 | 3.96E−05 |
| HMGB3 | 3.53E−09 | −0.522 | 0.235 | 0.269 | 4.02E−05 |
| TRIM14 | 3.72E−09 | −0.517 | 0.022 | 0.107 | 4.24E−05 |
| CCDC88A | 3.75E−09 | −0.317 | 0.059 | 0.164 | 4.27E−05 |
| NOTCH2 | 3.76E−09 | −0.590 | 0.079 | 0.18 | 4.29E−05 |
| SKIL | 3.83E−09 | −0.659 | 0.171 | 0.24 | 4.36E−05 |
| BUB3 | 3.87E−09 | −0.445 | 0.198 | 0.211 | 4.42E−05 |
| ADIPOR1 | 3.94E−09 | −0.414 | 0.185 | 0.18 | 4.50E−05 |
| GLRX2 | 4.01E−09 | −0.657 | 0.062 | 0.168 | 4.57E−05 |
| KPNA4 | 4.02E−09 | −0.609 | 0.177 | 0.256 | 4.58E−05 |
| STAG1 | 4.02E−09 | −0.644 | 0.1 | 0.203 | 4.59E−05 |
| MOB1A | 4.05E−09 | −0.283 | 0.211 | 0.158 | 4.61E−05 |
| PLBD1 | 4.08E−09 | −0.524 | 0.046 | 0.142 | 4.65E−05 |
| PRPSAP1 | 4.24E−09 | −0.627 | 0.082 | 0.149 | 4.83E−05 |
| SCNM1 | 4.28E−09 | −0.483 | 0.081 | 0.177 | 4.88E−05 |
| MAGEF1 | 4.35E−09 | −0.286 | 0.154 | 0.115 | 4.96E−05 |
| CMAS | 4.59E−09 | −0.538 | 0.128 | 0.186 | 5.23E−05 |
| ABHD11 | 4.85E−09 | −0.413 | 0.12 | 0.133 | 5.53E−05 |
| TALDO1 | 4.97E−09 | −0.522 | 0.146 | 0.171 | 5.66E−05 |
| FEN1 | 5.18E−09 | −0.476 | 0.052 | 0.121 | 5.90E−05 |
| NHP2 | 5.27E−09 | −0.288 | 0.372 | 0.32 | 6.01E−05 |
| CHD4 | 5.38E−09 | −0.346 | 0.361 | 0.351 | 6.14E−05 |
| TEX10 | 5.44E−09 | −0.609 | 0.095 | 0.171 | 6.20E−05 |
| KLF10 | 5.46E−09 | −0.583 | 0.097 | 0.174 | 6.22E−05 |
| PAPSS1 | 5.54E−09 | −0.581 | 0.106 | 0.183 | 6.31E−05 |
| PSME3 | 5.75E−09 | −0.487 | 0.076 | 0.173 | 6.56E−05 |
| PTDSS1 | 5.88E−09 | −0.559 | 0.106 | 0.167 | 6.71E−05 |
| MEX3A | 5.97E−09 | −0.545 | 0.044 | 0.135 | 6.81E−05 |
| ABLIM1 | 6.05E−09 | −0.536 | 0.157 | 0.199 | 6.89E−05 |
| CCNK | 6.05E−09 | −0.567 | 0.133 | 0.181 | 6.90E−05 |
| SLC25A4 | 6.29E−09 | −0.448 | 0.127 | 0.168 | 7.16E−05 |
| EMC10 | 6.80E−09 | −0.530 | 0.108 | 0.161 | 7.75E−05 |
| SHFM1 | 6.90E−09 | −0.257 | 0.333 | 0.276 | 7.87E−05 |
| MDH1 | 6.91E−09 | −0.424 | 0.231 | 0.219 | 7.88E−05 |
| MRPL19 | 7.31E−09 | −0.269 | 0.198 | 0.151 | 8.33E−05 |
| COMMD8 | 7.48E−09 | −0.612 | 0.048 | 0.124 | 8.52E−05 |
| CTSC | 7.57E−09 | −0.603 | 0.086 | 0.186 | 8.63E−05 |
| LSM2 | 7.62E−09 | −0.406 | 0.246 | 0.262 | 8.68E−05 |
| ATAD5 | 7.63E−09 | −0.438 | 0.022 | 0.102 | 8.70E−05 |
| SSRP1 | 7.64E−09 | −0.419 | 0.236 | 0.273 | 8.71E−05 |
| STAT3 | 8.06E−09 | −0.405 | 0.227 | 0.228 | 9.18E−05 |
| SSR3 | 8.40E−09 | −0.310 | 0.398 | 0.364 | 9.57E−05 |
| PA2G4 | 8.47E−09 | −0.419 | 0.333 | 0.37 | 9.65E−05 |
| C4orf48 | 8.54E−09 | −0.499 | 0.155 | 0.193 | 9.73E−05 |
| SZRD1 | 8.77E−09 | −0.306 | 0.168 | 0.139 | 1.00E−04 |
| SLBP | 8.79E−09 | −0.566 | 0.16 | 0.221 | 1.00E−04 |
| FAM98B | 9.05E−09 | −0.532 | 0.062 | 0.148 | 1.03E−04 |
| KDELR2 | 9.15E−09 | −0.422 | 0.276 | 0.303 | 1.04E−04 |
| RIMS2 | 9.50E−09 | −0.479 | 0.084 | 0.127 | 1.08E−04 |
| KXD1 | 9.74E−09 | −0.498 | 0.103 | 0.199 | 1.11E−04 |
| ATP11B | 9.77E−09 | −0.438 | 0.16 | 0.177 | 1.11E−04 |
| PAK2 | 9.81E−09 | −0.506 | 0.241 | 0.31 | 1.12E−04 |
| ATP5J2 | 9.93E−09 | −0.300 | 0.399 | 0.38 | 1.13E−04 |
| UBE2K | 1.06E−08 | −0.469 | 0.23 | 0.249 | 1.21E−04 |
| EBNA1BP2 | 1.08E−08 | −0.564 | 0.182 | 0.269 | 1.24E−04 |
| MZT2B | 1.10E−08 | −0.435 | 0.281 | 0.307 | 1.25E−04 |
| LYRM2 | 1.11E−08 | −0.578 | 0.111 | 0.222 | 1.27E−04 |
| RDH10 | 1.12E−08 | −0.546 | 0.033 | 0.118 | 1.28E−04 |
| UBXN2A | 1.23E−08 | −0.440 | 0.106 | 0.148 | 1.40E−04 |
| RIF1 | 1.25E−08 | −0.544 | 0.242 | 0.314 | 1.42E−04 |
| DYNC1H1 | 1.31E−08 | −0.523 | 0.276 | 0.333 | 1.49E−04 |
| POLR2F | 1.31E−08 | −0.412 | 0.235 | 0.243 | 1.50E−04 |
| DCUN1D5 | 1.35E−08 | −0.533 | 0.149 | 0.228 | 1.53E−04 |
| MAGOH | 1.35E−08 | −0.566 | 0.157 | 0.257 | 1.54E−04 |
| MX1 | 1.35E−08 | −0.665 | 0.105 | 0.215 | 1.54E−04 |
| MRPS21 | 1.35E−08 | −0.398 | 0.254 | 0.253 | 1.54E−04 |
| RPS6KB1 | 1.37E−08 | −0.540 | 0.082 | 0.175 | 1.57E−04 |
| DDA1 | 1.43E−08 | −0.338 | 0.198 | 0.18 | 1.63E−04 |
| CORO1C | 1.45E−08 | −0.503 | 0.041 | 0.136 | 1.65E−04 |
| ZMPSTE24 | 1.49E−08 | −0.476 | 0.181 | 0.225 | 1.70E−04 |
| PSME1 | 1.50E−08 | −0.314 | 0.301 | 0.257 | 1.71E−04 |
| CDKN3 | 1.51E−08 | −0.496 | 0.049 | 0.108 | 1.72E−04 |
| PRRC2B | 1.56E−08 | −0.274 | 0.225 | 0.193 | 1.77E−04 |
| C11orf54 | 1.60E−08 | −0.417 | 0.122 | 0.114 | 1.83E−04 |
| ARPC1B | 1.61E−08 | −0.403 | 0.236 | 0.206 | 1.83E−04 |
| TMEM33 | 1.62E−08 | −0.579 | 0.12 | 0.209 | 1.84E−04 |
| UBE2C | 1.64E−08 | −0.637 | 0.06 | 0.152 | 1.86E−04 |
| AHCTF1 | 1.64E−08 | −0.600 | 0.087 | 0.17 | 1.87E−04 |
| SPP1 | 1.73E−08 | −0.847 | 0.277 | 0.194 | 1.98E−04 |
| PSMA3 | 1.76E−08 | −0.331 | 0.295 | 0.335 | 2.00E−04 |
| FARSA | 1.77E−08 | −0.255 | 0.185 | 0.139 | 2.01E−04 |
| MAZ | 1.79E−08 | −0.538 | 0.109 | 0.173 | 2.04E−04 |
| ACTL6A | 1.82E−08 | −0.561 | 0.162 | 0.216 | 2.08E−04 |
| MCMBP | 1.83E−08 | −0.477 | 0.138 | 0.164 | 2.09E−04 |
| IRAK4 | 1.85E−08 | −0.414 | 0.092 | 0.11 | 2.11E−04 |
| PFDN2 | 1.91E−08 | −0.505 | 0.144 | 0.241 | 2.18E−04 |
| RNF114 | 1.92E−08 | −0.268 | 0.176 | 0.137 | 2.19E−04 |
| SMYD2 | 1.97E−08 | −0.409 | 0.024 | 0.102 | 2.24E−04 |
| C1orf35 | 1.97E−08 | −0.549 | 0.106 | 0.161 | 2.25E−04 |
| PSMD8 | 1.98E−08 | −0.284 | 0.311 | 0.279 | 2.26E−04 |
| HIST1H1C | 2.00E−08 | −0.447 | 0.288 | 0.31 | 2.28E−04 |
| SPINT2 | 2.04E−08 | −0.300 | 0.504 | 0.482 | 2.33E−04 |
| KEAP1 | 2.05E−08 | −0.496 | 0.067 | 0.17 | 2.34E−04 |
| ZCRB1 | 2.09E−08 | −0.368 | 0.25 | 0.234 | 2.38E−04 |
| NOTCH1 | 2.14E−08 | −0.476 | 0.03 | 0.101 | 2.44E−04 |
| SCRN1 | 2.20E−08 | −0.418 | 0.141 | 0.175 | 2.50E−04 |
| PGD | 2.20E−08 | −0.525 | 0.089 | 0.133 | 2.51E−04 |
| EMP1 | 2.23E−08 | −0.474 | 0.326 | 0.335 | 2.54E−04 |
| RNF2 | 2.36E−08 | −0.508 | 0.038 | 0.12 | 2.69E−04 |
| SPTAN1 | 2.53E−08 | −0.550 | 0.092 | 0.17 | 2.89E−04 |
| RAD23B | 2.56E−08 | −0.451 | 0.244 | 0.285 | 2.92E−04 |
| MT-ND2 | 2.65E−08 | −0.463 | 0.832 | 0.846 | 3.02E−04 |
| CTC-444N24.11 | 2.76E−08 | −0.390 | 0.106 | 0.136 | 3.14E−04 |
| COPE | 2.80E−08 | −0.407 | 0.223 | 0.222 | 3.19E−04 |
| PSMB5 | 2.89E−08 | −0.413 | 0.277 | 0.278 | 3.29E−04 |
| TXNDC12 | 2.89E−08 | −0.366 | 0.179 | 0.183 | 3.29E−04 |
| PNO1 | 2.98E−08 | −0.419 | 0.176 | 0.175 | 3.39E−04 |
| TRAPPC2L | 3.15E−08 | −0.304 | 0.122 | 0.099 | 3.59E−04 |
| IRF6 | 3.19E−08 | −0.515 | 0.071 | 0.117 | 3.64E−04 |
| TNFSF13B | 3.21E−08 | −0.547 | 0.029 | 0.102 | 3.66E−04 |
| WBP11 | 3.23E−08 | −0.363 | 0.29 | 0.256 | 3.68E−04 |
| C7orf73 | 3.25E−08 | −0.442 | 0.19 | 0.234 | 3.71E−04 |
| UBE2Q1 | 3.33E−08 | −0.506 | 0.067 | 0.168 | 3.79E−04 |
| SRP14 | 3.36E−08 | −0.288 | 0.45 | 0.402 | 3.83E−04 |
| C1orf43 | 3.38E−08 | −0.311 | 0.273 | 0.27 | 3.85E−04 |
| UFC1 | 3.58E−08 | −0.407 | 0.263 | 0.273 | 4.07E−04 |
| PAXBP1 | 3.64E−08 | −0.533 | 0.07 | 0.117 | 4.15E−04 |
| MRPL47 | 3.72E−08 | −0.551 | 0.174 | 0.27 | 4.24E−04 |
| PNKD | 3.77E−08 | −0.286 | 0.181 | 0.152 | 4.30E−04 |
| CDC37 | 3.84E−08 | −0.529 | 0.146 | 0.23 | 4.37E−04 |
| ACSL4 | 4.15E−08 | −0.270 | 0.025 | 0.102 | 4.73E−04 |
| MT-ND5 | 4.20E−08 | −0.394 | 0.604 | 0.592 | 4.79E−04 |
| C11orf48 | 4.27E−08 | −0.371 | 0.166 | 0.158 | 4.87E−04 |
| TXNIP | 4.52E−08 | −0.382 | 0.166 | 0.287 | 5.15E−04 |
| DOCK5 | 4.64E−08 | −0.584 | 0.038 | 0.118 | 5.28E−04 |
| SRP9 | 4.70E−08 | −0.396 | 0.372 | 0.393 | 5.36E−04 |
| PPP1R7 | 4.91E−08 | −0.292 | 0.193 | 0.158 | 5.59E−04 |
| ANKRD42 | 4.93E−08 | −0.336 | 0.174 | 0.174 | 5.62E−04 |
| PPM1G | 5.01E−08 | −0.414 | 0.296 | 0.297 | 5.71E−04 |
| GAR1 | 5.11E−08 | −0.584 | 0.092 | 0.164 | 5.82E−04 |
| SLC39A14 | 5.45E−08 | −0.544 | 0.052 | 0.143 | 6.22E−04 |
| MT-ND1 | 5.47E−08 | −0.379 | 0.708 | 0.67 | 6.24E−04 |
| PUS7L | 5.50E−08 | −0.671 | 0.079 | 0.162 | 6.26E−04 |
| BPNT1 | 5.57E−08 | −0.610 | 0.051 | 0.133 | 6.34E−04 |
| BRK1 | 5.63E−08 | −0.323 | 0.255 | 0.241 | 6.42E−04 |
| PRELID1 | 5.91E−08 | −0.409 | 0.287 | 0.265 | 6.73E−04 |
| PAFAH1B1 | 6.15E−08 | −0.486 | 0.177 | 0.224 | 7.01E−04 |
| HNRNPA1L2 | 6.15E−08 | −0.409 | 0.036 | 0.101 | 7.01E−04 |
| GLRX3 | 6.19E−08 | −0.397 | 0.179 | 0.208 | 7.06E−04 |
| ARL6IP6 | 6.22E−08 | −0.400 | 0.035 | 0.117 | 7.08E−04 |
| NDRG2 | 6.25E−08 | −0.449 | 0.073 | 0.108 | 7.12E−04 |
| PFN2 | 6.28E−08 | −0.324 | 0.155 | 0.146 | 7.16E−04 |
| UTP6 | 6.34E−08 | −0.570 | 0.073 | 0.154 | 7.23E−04 |
| ERP29 | 6.39E−08 | −0.335 | 0.225 | 0.208 | 7.28E−04 |
| ZNF664 | 6.55E−08 | −0.464 | 0.132 | 0.187 | 7.47E−04 |
| C7orf50 | 6.76E−08 | −0.333 | 0.261 | 0.221 | 7.71E−04 |
| PRRC1 | 7.11E−08 | −0.325 | 0.138 | 0.121 | 8.10E−04 |
| UBE2I | 7.26E−08 | −0.401 | 0.252 | 0.263 | 8.28E−04 |
| IPO9 | 7.32E−08 | −0.579 | 0.081 | 0.17 | 8.34E−04 |
| MYO9B | 7.33E−08 | −0.519 | 0.057 | 0.146 | 8.36E−04 |
| SHMT2 | 7.59E−08 | −0.298 | 0.119 | 0.114 | 8.65E−04 |
| RANBP1 | 7.71E−08 | −0.294 | 0.32 | 0.304 | 8.79E−04 |
| POLD2 | 7.79E−08 | −0.532 | 0.09 | 0.156 | 8.88E−04 |
| TMED1 | 7.82E−08 | −0.448 | 0.043 | 0.129 | 8.91E−04 |
| RPL39L | 7.83E−08 | −0.559 | 0.147 | 0.183 | 8.92E−04 |
| ZNF121 | 8.12E−08 | −0.534 | 0.092 | 0.177 | 9.25E−04 |
| FGD4 | 8.13E−08 | −0.513 | 0.033 | 0.114 | 9.27E−04 |
| MORF4L2 | 8.24E−08 | −0.255 | 0.391 | 0.327 | 9.39E−04 |
| MT-TS2 | 8.25E−08 | −0.889 | 0.249 | 0.301 | 9.40E−04 |
| CDCA7L | 8.29E−08 | −0.484 | 0.071 | 0.121 | 9.45E−04 |
| UROD | 8.37E−08 | −0.547 | 0.074 | 0.13 | 9.54E−04 |
| MTAP | 8.49E−08 | −0.535 | 0.067 | 0.155 | 9.68E−04 |
| NCK2 | 8.56E−08 | −0.457 | 0.067 | 0.113 | 9.75E−04 |
| MCM6 | 8.81E−08 | −0.557 | 0.067 | 0.142 | 1.00E−03 |
| SRPK1 | 8.90E−08 | −0.401 | 0.2 | 0.196 | 1.01E−03 |
| GNA13 | 8.99E−08 | −0.501 | 0.052 | 0.142 | 1.02E−03 |
| CALM3 | 9.07E−08 | −0.496 | 0.22 | 0.291 | 1.03E−03 |
| GTF2A2 | 9.14E−08 | −0.265 | 0.217 | 0.186 | 1.04E−03 |
| SNRPC | 9.50E−08 | −0.278 | 0.279 | 0.257 | 1.08E−03 |
| C19orf48 | 1.04E−07 | −0.387 | 0.158 | 0.192 | 1.19E−03 |
| PPP1CC | 1.09E−07 | −0.385 | 0.211 | 0.224 | 1.24E−03 |
| LINC00152 | 1.15E−07 | −0.389 | 0.07 | 0.174 | 1.31E−03 |
| NCBP2 | 1.16E−07 | −0.281 | 0.304 | 0.275 | 1.32E−03 |
| MLLT4 | 1.16E−07 | −0.547 | 0.119 | 0.175 | 1.33E−03 |
| RFXANK | 1.17E−07 | −0.425 | 0.067 | 0.123 | 1.34E−03 |
| IFI16 | 1.17E−07 | −0.645 | 0.062 | 0.148 | 1.34E−03 |
| SIGMAR1 | 1.19E−07 | −0.401 | 0.086 | 0.133 | 1.35E−03 |
| MCM7 | 1.19E−07 | −0.525 | 0.068 | 0.14 | 1.36E−03 |
| DNAH14 | 1.24E−07 | −0.548 | 0.097 | 0.197 | 1.41E−03 |
| LAGE3 | 1.24E−07 | −0.339 | 0.176 | 0.167 | 1.42E−03 |
| TTK | 1.38E−07 | −0.482 | 0.032 | 0.101 | 1.57E−03 |
| RBBP7 | 1.38E−07 | −0.265 | 0.208 | 0.167 | 1.57E−03 |
| C19orf10 | 1.42E−07 | −0.482 | 0.209 | 0.254 | 1.62E−03 |
| PDE4DIP | 1.42E−07 | −0.505 | 0.049 | 0.118 | 1.62E−03 |
| YIF1A | 1.52E−07 | −0.447 | 0.13 | 0.151 | 1.74E−03 |
| LARP4B | 1.55E−07 | −0.537 | 0.106 | 0.155 | 1.76E−03 |
| PDIA4 | 1.57E−07 | −0.353 | 0.274 | 0.273 | 1.79E−03 |
| NUP160 | 1.59E−07 | −0.391 | 0.036 | 0.121 | 1.81E−03 |
| NAA15 | 1.64E−07 | −0.490 | 0.185 | 0.219 | 1.87E−03 |
| RABIF | 1.65E−07 | −0.461 | 0.055 | 0.105 | 1.88E−03 |
| CNTLN | 1.66E−07 | −0.501 | 0.032 | 0.108 | 1.89E−03 |
| CISD2 | 1.71E−07 | −0.333 | 0.155 | 0.146 | 1.95E−03 |
| C19orf60 | 1.72E−07 | −0.432 | 0.135 | 0.174 | 1.96E−03 |
| BRCC3 | 1.73E−07 | −0.514 | 0.127 | 0.152 | 1.97E−03 |
| RFC2 | 1.73E−07 | −0.383 | 0.092 | 0.11 | 1.97E−03 |
| GPATCH4 | 1.78E−07 | −0.441 | 0.082 | 0.18 | 2.03E−03 |
| BAG6 | 1.80E−07 | −0.416 | 0.138 | 0.156 | 2.06E−03 |
| ARHGDIB | 1.89E−07 | −0.339 | 0.227 | 0.222 | 2.16E−03 |
| TRIM28 | 1.91E−07 | −0.329 | 0.157 | 0.168 | 2.17E−03 |
| UBA2 | 1.94E−07 | −0.474 | 0.155 | 0.208 | 2.21E−03 |
| CKAP5 | 1.96E−07 | −0.538 | 0.127 | 0.164 | 2.23E−03 |
| TIMM13 | 2.00E−07 | −0.263 | 0.311 | 0.268 | 2.28E−03 |
| CLIC1 | 2.09E−07 | −0.425 | 0.174 | 0.241 | 2.38E−03 |
| CBX1 | 2.13E−07 | −0.389 | 0.211 | 0.206 | 2.43E−03 |
| LHCGR | 2.17E−07 | −0.436 | 0.055 | 0.142 | 2.47E−03 |
| SMC2 | 2.19E−07 | −0.510 | 0.141 | 0.177 | 2.50E−03 |
| EI24 | 2.22E−07 | −0.322 | 0.17 | 0.161 | 2.53E−03 |
| ATF6 | 2.36E−07 | −0.291 | 0.216 | 0.184 | 2.69E−03 |
| HSD17B10 | 2.51E−07 | −0.439 | 0.13 | 0.173 | 2.86E−03 |
| JUP | 2.55E−07 | −0.476 | 0.171 | 0.216 | 2.91E−03 |
| RAP1A | 2.59E−07 | −0.334 | 0.163 | 0.154 | 2.95E−03 |
| SLC4A7 | 2.60E−07 | −0.462 | 0.07 | 0.173 | 2.96E−03 |
| ATP1A1 | 2.60E−07 | −0.328 | 0.311 | 0.332 | 2.97E−03 |
| FANCI | 2.61E−07 | −0.528 | 0.074 | 0.146 | 2.97E−03 |
| RNY1 | 2.63E−07 | −0.338 | 0.147 | 0.165 | 3.00E−03 |
| SNRPN | 2.65E−07 | −0.520 | 0.036 | 0.115 | 3.02E−03 |
| SHOC2 | 2.68E−07 | −0.353 | 0.098 | 0.101 | 3.05E−03 |
| PMVK | 2.70E−07 | −0.295 | 0.122 | 0.11 | 3.08E−03 |
| LYPLA1 | 2.71E−07 | −0.437 | 0.216 | 0.268 | 3.08E−03 |
| KBTBD2 | 2.72E−07 | −0.506 | 0.032 | 0.102 | 3.09E−03 |
| TRAPPC3 | 2.74E−07 | −0.364 | 0.136 | 0.137 | 3.12E−03 |
| EBAG9 | 2.78E−07 | −0.321 | 0.23 | 0.202 | 3.16E−03 |
| SMC1A | 2.79E−07 | −0.552 | 0.144 | 0.215 | 3.18E−03 |
| ABT1 | 2.92E−07 | −0.280 | 0.125 | 0.108 | 3.33E−03 |
| CHSY1 | 2.97E−07 | −0.485 | 0.044 | 0.114 | 3.38E−03 |
| SOD1 | 3.03E−07 | −0.311 | 0.345 | 0.368 | 3.46E−03 |
| FAM120A | 3.12E−07 | −0.267 | 0.231 | 0.187 | 3.56E−03 |
| TIMM23 | 3.17E−07 | −0.415 | 0.074 | 0.114 | 3.61E−03 |
| LINC00665 | 3.21E−07 | −0.420 | 0.097 | 0.123 | 3.66E−03 |
| COMMD2 | 3.22E−07 | −0.605 | 0.094 | 0.187 | 3.67E−03 |
| EIF4A1 | 3.30E−07 | −0.362 | 0.223 | 0.234 | 3.76E−03 |
| NT5C3A | 3.34E−07 | −0.502 | 0.128 | 0.194 | 3.81E−03 |
| SPTBN1 | 3.38E−07 | −0.379 | 0.212 | 0.205 | 3.85E−03 |
| NOP10 | 3.38E−07 | −0.431 | 0.238 | 0.26 | 3.85E−03 |
| UBQLN1 | 3.49E−07 | −0.445 | 0.216 | 0.23 | 3.98E−03 |
| POLR2J | 3.52E−07 | −0.352 | 0.201 | 0.193 | 4.01E−03 |
| GGPS1 | 3.53E−07 | −0.311 | 0.182 | 0.159 | 4.02E−03 |
| ARL4C | 3.56E−07 | −0.473 | 0.082 | 0.124 | 4.06E−03 |
| SLC39A4 | 3.58E−07 | −0.260 | 0.154 | 0.135 | 4.09E−03 |
| PEA15 | 3.67E−07 | −0.541 | 0.192 | 0.244 | 4.18E−03 |
| RDX | 3.76E−07 | −0.332 | 0.268 | 0.26 | 4.29E−03 |
| GTF3C6 | 3.82E−07 | −0.318 | 0.139 | 0.259 | 4.36E−03 |
| SEC31A | 3.83E−07 | −0.289 | 0.235 | 0.199 | 4.37E−03 |
| PAQR3 | 4.15E−07 | −0.354 | 0.032 | 0.11 | 4.73E−03 |
| REXO2 | 4.24E−07 | −0.480 | 0.106 | 0.178 | 4.83E−03 |
| TBCB | 4.30E−07 | −0.489 | 0.144 | 0.221 | 4.90E−03 |
| KLHL23 | 4.30E−07 | −0.506 | 0.063 | 0.149 | 4.90E−03 |
| MBNL1 | 4.33E−07 | −0.452 | 0.171 | 0.259 | 4.94E−03 |
| DHX9 | 4.51E−07 | −0.483 | 0.254 | 0.298 | 5.14E−03 |
| SAV1 | 4.51E−07 | −0.501 | 0.094 | 0.161 | 5.14E−03 |
| ATP5G1 | 4.60E−07 | −0.283 | 0.204 | 0.186 | 5.24E−03 |
| XPO1 | 4.60E−07 | −0.316 | 0.239 | 0.23 | 5.24E−03 |
| RPP38 | 4.62E−07 | −0.523 | 0.073 | 0.114 | 5.26E−03 |
| LPGAT1 | 4.68E−07 | −0.526 | 0.151 | 0.219 | 5.34E−03 |
| AP2M1 | 4.86E−07 | −0.385 | 0.231 | 0.257 | 5.54E−03 |
| HIST1H1D | 4.95E−07 | −0.597 | 0.095 | 0.171 | 5.64E−03 |
| SDHC | 5.03E−07 | −0.333 | 0.114 | 0.113 | 5.73E−03 |
| RSRC1 | 5.15E−07 | −0.283 | 0.242 | 0.202 | 5.87E−03 |
| TCOF1 | 5.24E−07 | −0.422 | 0.128 | 0.111 | 5.97E−03 |
| GNAI2 | 5.57E−07 | −0.430 | 0.071 | 0.139 | 6.35E−03 |
| PMEPA1 | 5.68E−07 | −0.557 | 0.109 | 0.197 | 6.47E−03 |
| TYMS | 5.75E−07 | −0.428 | 0.054 | 0.136 | 6.55E−03 |
| TIMM44 | 5.80E−07 | −0.524 | 0.16 | 0.213 | 6.61E−03 |
| ACLY | 5.90E−07 | −0.419 | 0.152 | 0.187 | 6.72E−03 |
| CTNNAL1 | 6.02E−07 | −0.537 | 0.054 | 0.12 | 6.86E−03 |
| NLN | 6.03E−07 | −0.502 | 0.09 | 0.174 | 6.87E−03 |
| HDGF | 6.42E−07 | −0.335 | 0.349 | 0.355 | 7.32E−03 |
| TP53BP2 | 6.65E−07 | −0.516 | 0.082 | 0.123 | 7.58E−03 |
| HGSNAT | 6.75E−07 | −0.458 | 0.032 | 0.104 | 7.70E−03 |
| CAND1 | 7.00E−07 | −0.439 | 0.146 | 0.184 | 7.98E−03 |
| RAD51AP1 | 7.02E−07 | −0.473 | 0.057 | 0.129 | 8.00E−03 |
| SLC25A32 | 7.11E−07 | −0.415 | 0.068 | 0.139 | 8.10E−03 |
| MRPS18C | 7.34E−07 | −0.476 | 0.158 | 0.219 | 8.37E−03 |
| CCDC167 | 7.39E−07 | −0.309 | 0.158 | 0.135 | 8.42E−03 |
| ASNSD1 | 7.47E−07 | −0.424 | 0.201 | 0.202 | 8.51E−03 |
| LMNB2 | 7.48E−07 | −0.504 | 0.051 | 0.114 | 8.52E−03 |
| PRKAR1A | 7.50E−07 | −0.408 | 0.216 | 0.238 | 8.55E−03 |
| HUWE1 | 7.56E−07 | −0.414 | 0.255 | 0.273 | 8.61E−03 |
| ZNF106 | 7.57E−07 | −0.283 | 0.198 | 0.161 | 8.63E−03 |
| TTL | 7.94E−07 | −0.429 | 0.057 | 0.127 | 9.05E−03 |
| FAM136A | 7.96E−07 | −0.380 | 0.158 | 0.152 | 9.07E−03 |
| SDCCAG8 | 8.05E−07 | −0.483 | 0.098 | 0.167 | 9.18E−03 |
| CD320 | 8.36E−07 | −0.437 | 0.033 | 0.104 | 9.53E−03 |
| RCHY1 | 8.40E−07 | −0.419 | 0.057 | 0.136 | 9.57E−03 |
| FNDC3B | 8.71E−07 | −0.559 | 0.095 | 0.173 | 9.93E−03 |
| GARS | 8.85E−07 | −0.309 | 0.258 | 0.246 | 1.01E−02 |
| UTP11L | 9.11E−07 | −0.463 | 0.189 | 0.216 | 1.04E−02 |
| AHSA1 | 9.37E−07 | −0.386 | 0.165 | 0.189 | 1.07E−02 |
| BROX | 9.39E−07 | −0.578 | 0.179 | 0.235 | 1.07E−02 |
| COX16 | 9.54E−07 | −0.251 | 0.344 | 0.316 | 1.09E−02 |
| IFRD1 | 1.01E−06 | −0.526 | 0.13 | 0.197 | 1.15E−02 |
| FUCA2 | 1.02E−06 | −0.332 | 0.079 | 0.178 | 1.17E−02 |
| MED13 | 1.02E−06 | −0.489 | 0.146 | 0.241 | 1.17E−02 |
| DCUN1D4 | 1.06E−06 | −0.435 | 0.067 | 0.155 | 1.21E−02 |
| TSC22D1 | 1.13E−06 | −0.326 | 0.301 | 0.273 | 1.29E−02 |
| ABI1 | 1.14E−06 | −0.325 | 0.171 | 0.149 | 1.30E−02 |
| FIP1L1 | 1.19E−06 | −0.445 | 0.127 | 0.181 | 1.35E−02 |
| ETV3 | 1.19E−06 | −0.375 | 0.098 | 0.111 | 1.36E−02 |
| BBX | 1.20E−06 | −0.342 | 0.197 | 0.168 | 1.36E−02 |
| UHMK1 | 1.24E−06 | −0.462 | 0.162 | 0.205 | 1.41E−02 |
| PTPMT1 | 1.27E−06 | −0.362 | 0.111 | 0.114 | 1.45E−02 |
| SMEK1 | 1.27E−06 | −0.493 | 0.165 | 0.24 | 1.45E−02 |
| LAMTOR2 | 1.27E−06 | −0.393 | 0.17 | 0.197 | 1.45E−02 |
| WBP5 | 1.33E−06 | −0.436 | 0.157 | 0.19 | 1.52E−02 |
| HEATR1 | 1.36E−06 | −0.533 | 0.108 | 0.196 | 1.55E−02 |
| SAE1 | 1.44E−06 | −0.299 | 0.138 | 0.135 | 1.64E−02 |
| FRYL | 1.44E−06 | −0.460 | 0.146 | 0.186 | 1.64E−02 |
| SMARCA4 | 1.47E−06 | −0.556 | 0.141 | 0.216 | 1.67E−02 |
| RSU1 | 1.49E−06 | −0.506 | 0.044 | 0.124 | 1.70E−02 |
| PDXK | 1.53E−06 | −0.449 | 0.173 | 0.209 | 1.74E−02 |
| 10-Sep | 1.64E−06 | −0.393 | 0.079 | 0.118 | 1.87E−02 |
| EIF4A3 | 1.66E−06 | −0.445 | 0.193 | 0.234 | 1.90E−02 |
| TCP1 | 1.78E−06 | −0.278 | 0.287 | 0.26 | 2.03E−02 |
| ROCK1 | 1.79E−06 | −0.318 | 0.282 | 0.3 | 2.04E−02 |
| MRPL22 | 1.80E−06 | −0.266 | 0.216 | 0.177 | 2.06E−02 |
| PRPS2 | 1.81E−06 | −0.450 | 0.049 | 0.129 | 2.07E−02 |
| GART | 1.82E−06 | −0.401 | 0.111 | 0.192 | 2.07E−02 |
| MED10 | 1.84E−06 | −0.597 | 0.097 | 0.167 | 2.09E−02 |
| PDCD4 | 1.92E−06 | −0.269 | 0.187 | 0.148 | 2.19E−02 |
| SAP30BP | 1.93E−06 | −0.355 | 0.173 | 0.171 | 2.20E−02 |
| RGS2 | 1.94E−06 | −0.503 | 0.13 | 0.143 | 2.21E−02 |
| LEPROTL1 | 1.96E−06 | −0.362 | 0.139 | 0.133 | 2.23E−02 |
| AGPAT5 | 1.96E−06 | −0.535 | 0.092 | 0.186 | 2.24E−02 |
| MPLKIP | 2.02E−06 | −0.438 | 0.147 | 0.156 | 2.31E−02 |
| MAPK1IP1L | 2.04E−06 | −0.457 | 0.119 | 0.197 | 2.33E−02 |
| UQCRFS1 | 2.05E−06 | −0.451 | 0.162 | 0.216 | 2.34E−02 |
| IFNAR1 | 2.10E−06 | −0.540 | 0.133 | 0.193 | 2.40E−02 |
| AP1S2 | 2.14E−06 | −0.475 | 0.052 | 0.104 | 2.44E−02 |
| DTD1 | 2.16E−06 | −0.448 | 0.09 | 0.173 | 2.47E−02 |
| PUM2 | 2.17E−06 | −0.451 | 0.173 | 0.206 | 2.47E−02 |
| PAWR | 2.23E−06 | −0.555 | 0.124 | 0.209 | 2.55E−02 |
| ELAVL1 | 2.30E−06 | −0.431 | 0.116 | 0.174 | 2.62E−02 |
| RRBP1 | 2.35E−06 | −0.393 | 0.33 | 0.349 | 2.68E−02 |
| PSAT1 | 2.37E−06 | −0.445 | 0.067 | 0.107 | 2.70E−02 |
| MRPS14 | 2.39E−06 | −0.492 | 0.127 | 0.205 | 2.73E−02 |
| FH | 2.41E−06 | −0.503 | 0.109 | 0.149 | 2.75E−02 |
| UGCG | 2.50E−06 | −0.508 | 0.103 | 0.158 | 2.85E−02 |
| MRPL9 | 2.54E−06 | −0.411 | 0.098 | 0.186 | 2.90E−02 |
| HADH | 2.64E−06 | −0.284 | 0.125 | 0.105 | 3.01E−02 |
| MARS | 2.85E−06 | −0.281 | 0.124 | 0.118 | 3.25E−02 |
| DDX42 | 2.94E−06 | −0.297 | 0.185 | 0.181 | 3.35E−02 |
| TCF3 | 2.95E−06 | −0.458 | 0.073 | 0.143 | 3.36E−02 |
| CYFIP1 | 2.95E−06 | −0.409 | 0.095 | 0.117 | 3.36E−02 |
| AKIRIN2 | 3.03E−06 | −0.406 | 0.035 | 0.104 | 3.45E−02 |
| RPN1 | 3.09E−06 | −0.382 | 0.238 | 0.281 | 3.52E−02 |
| PSMD14 | 3.17E−06 | −0.319 | 0.284 | 0.298 | 3.62E−02 |
| RAB8A | 3.20E−06 | −0.301 | 0.184 | 0.17 | 3.65E−02 |
| NUCB1 | 3.31E−06 | −0.314 | 0.165 | 0.155 | 3.77E−02 |
| PSMB8 | 3.33E−06 | −0.499 | 0.09 | 0.137 | 3.79E−02 |
| YME1L1 | 3.55E−06 | −0.366 | 0.233 | 0.231 | 4.05E−02 |
| CEP57 | 3.64E−06 | −0.405 | 0.141 | 0.19 | 4.15E−02 |
| NAA50 | 3.75E−06 | −0.354 | 0.227 | 0.208 | 4.28E−02 |
| NCOA4 | 3.76E−06 | −0.307 | 0.16 | 0.146 | 4.28E−02 |
| ZBTB38 | 3.80E−06 | −0.400 | 0.147 | 0.154 | 4.33E−02 |
| YWHAH | 3.81E−06 | −0.302 | 0.17 | 0.203 | 4.35E−02 |
| LRPPRC | 3.96E−06 | −0.252 | 0.311 | 0.272 | 4.51E−02 |
| MRPL32 | 3.99E−06 | −0.451 | 0.117 | 0.215 | 4.55E−02 |
| MRPL16 | 4.08E−06 | −0.409 | 0.073 | 0.129 | 4.65E−02 |
| PSMD4 | 4.18E−06 | −0.366 | 0.201 | 0.257 | 4.76E−02 |
| OS9 | 4.27E−06 | −0.364 | 0.163 | 0.175 | 4.87E−02 |
| FLAD1 | 4.38E−06 | −0.449 | 0.041 | 0.108 | 5.00E−02 |

CSF_full.r2.set.RvNR.tumor.post.bimod.markers.up.1

| | | | | | |
|---|---|---|---|---|---|
| MGP | 0.00E+00 | 4.705 | 0.878 | 0.012 | 0.00E+00 |
| SLPI | 0.00E+00 | 3.437 | 0.74 | 0.004 | 0.00E+00 |
| GLYATL2 | 0.00E+00 | 3.273 | 0.756 | 0.002 | 0.00E+00 |
| MTRNR2L8 | 0.00E+00 | 2.330 | 0.666 | 0.552 | 0.00E+00 |
| PIGR | 2.30E−305 | 3.174 | 0.681 | 0.003 | 2.62E−301 |
| MIF | 6.10E−303 | 1.602 | 0.509 | 0.497 | 6.95E−299 |
| MTRNR2L1 | 4.76E−289 | 1.982 | 0.854 | 0.698 | 5.43E−285 |
| EPCAM | 1.44E−262 | 2.550 | 0.645 | 0.014 | 1.64E−258 |
| MTRNR2L3 | 2.90E−253 | 1.747 | 0.569 | 0.487 | 3.31E−249 |
| MT-CO1 | 2.42E−238 | 1.962 | 0.952 | 0.722 | 2.76E−234 |
| HAP1 | 6.95E−233 | 0.771 | 0.334 | 0.585 | 7.92E−229 |
| TACSTD2 | 9.65E−228 | 2.382 | 0.572 | 0.011 | 1.10E−223 |
| KRT19 | 1.24E−222 | 1.939 | 0.584 | 0.017 | 1.42E−218 |
| RPS27 | 7.28E−216 | 1.185 | 0.415 | 0.52 | 8.30E−212 |
| MTRNR2L2 | 3.25E−214 | 1.198 | 0.516 | 0.614 | 3.70E−210 |
| MDK | 4.74E−213 | 2.300 | 0.522 | 0.001 | 5.41E−209 |
| FXYD3 | 7.40E−205 | 2.313 | 0.547 | 0.014 | 8.44E−201 |
| LCN2 | 1.27E−204 | 2.434 | 0.506 | 0.001 | 1.45E−200 |
| RPL10 | 1.44E−201 | 1.174 | 0.449 | 0.504 | 1.64E−197 |
| MTRNR2L12 | 2.92E−196 | 0.637 | 0.322 | 0.612 | 3.33E−192 |
| MT-CO3 | 8.46E−190 | 1.328 | 0.972 | 0.716 | 9.64E−186 |
| RPS15 | 1.43E−189 | 1.122 | 0.573 | 0.622 | 1.63E−185 |
| COX6A1P2 | 3.83E−188 | 0.601 | 0.189 | 0.417 | 4.36E−184 |
| MT-CO2 | 2.39E−180 | 1.520 | 0.865 | 0.677 | 2.73E−176 |
| MTRNR2L6 | 1.12E−170 | 0.606 | 0.211 | 0.439 | 1.28E−166 |
| RPL23A | 2.16E−159 | 0.802 | 0.366 | 0.522 | 2.46E−155 |
| RPL12 | 1.35E−158 | 2.714 | 0.725 | 0.752 | 1.54E−154 |
| RPS15A | 3.43E−156 | 1.065 | 0.81 | 0.795 | 3.91E−152 |
| RPL28 | 8.69E−152 | 0.402 | 0.507 | 0.718 | 9.91E−148 |
| MT-ND4L | 1.11E−150 | 1.635 | 0.776 | 0.555 | 1.27E−146 |
| KRT23 | 1.37E−149 | 2.040 | 0.435 | 0.013 | 1.56E−145 |
| DSG2 | 1.70E−149 | 1.792 | 0.397 | 0.002 | 1.93E−145 |
| MTRNR2L5 | 1.33E−148 | 0.258 | 0.084 | 0.315 | 1.52E−144 |
| RPS9 | 3.18E−147 | 0.622 | 0.501 | 0.628 | 3.62E−143 |
| RPL29 | 7.26E−144 | 0.821 | 0.313 | 0.424 | 8.28E−140 |
| RPL41 | 5.80E−140 | 0.322 | 0.541 | 0.777 | 6.61E−136 |
| RPS27A | 1.16E−138 | 1.008 | 0.875 | 0.805 | 1.32E−134 |
| H3F3A | 3.29E−137 | 0.728 | 0.144 | 0.203 | 3.75E−133 |
| RPL6 | 1.12E−136 | 0.420 | 0.377 | 0.602 | 1.28E−132 |
| RPL37 | 4.27E−134 | 0.923 | 0.732 | 0.827 | 4.87E−130 |
| EEF1B2 | 8.75E−134 | 0.469 | 0.442 | 0.625 | 9.97E−130 |
| RPS7 | 7.60E−133 | 0.616 | 0.273 | 0.449 | 8.66E−129 |
| RPL34 | 9.23E−133 | 1.037 | 0.907 | 0.814 | 1.05E−128 |
| RPL13A | 1.58E−132 | 0.824 | 0.819 | 0.849 | 1.81E−128 |
| MTRNR2L7 | 4.30E−131 | 0.327 | 0.065 | 0.229 | 4.91E−127 |
| RPL36 | 3.26E−129 | 0.864 | 0.589 | 0.664 | 3.72E−125 |
| SNRPG | 4.42E−129 | 0.694 | 0.301 | 0.465 | 5.04E−125 |
| TMA7 | 1.99E−128 | 0.693 | 0.289 | 0.443 | 2.26E−124 |
| RPL7A | 2.69E−125 | 0.354 | 0.537 | 0.726 | 3.07E−121 |
| RPS2 | 7.35E−125 | 0.382 | 0.564 | 0.7 | 8.37E−121 |
| C11orf31 | 8.81E−125 | 0.670 | 0.324 | 0.465 | 1.00E−120 |
| IFI27L2 | 2.47E−124 | 1.016 | 0.209 | 0.179 | 2.82E−120 |
| CENPW | 1.19E−123 | 1.882 | 0.432 | 0.039 | 1.35E−119 |
| NPM1 | 1.82E−123 | 0.525 | 0.513 | 0.662 | 2.08E−119 |
| ATP5EP2 | 2.13E−123 | 0.724 | 0.256 | 0.381 | 2.42E−119 |
| WFDC2 | 1.23E−121 | 1.579 | 0.333 | 0.001 | 1.40E−117 |
| PPIA | 3.60E−120 | 0.729 | 0.486 | 0.622 | 4.10E−116 |
| RPL26 | 4.35E−116 | 0.557 | 0.451 | 0.692 | 4.96E−112 |
| RPSA | 7.82E−116 | 0.325 | 0.357 | 0.593 | 8.91E−112 |
| IFITM1 | 1.92E−114 | 0.981 | 0.281 | 0.274 | 2.18E−110 |
| S100A1 | 6.41E−114 | 0.425 | 0.277 | 0.484 | 7.31E−110 |
| TCEB2 | 4.80E−113 | 0.819 | 0.494 | 0.531 | 5.47E−109 |
| MT-RNR2 | 5.39E−113 | 1.008 | 0.999 | 0.989 | 6.14E−109 |
| VTCN1 | 8.83E−113 | 1.575 | 0.31 | 0.001 | 1.01E−108 |
| NDUFB11 | 1.38E−112 | 0.674 | 0.312 | 0.46 | 1.57E−108 |
| RPL22 | 4.40E−111 | 0.287 | 0.338 | 0.576 | 5.02E−107 |
| HSPB1 | 1.07E−110 | 0.586 | 0.308 | 0.47 | 1.22E−106 |
| HSPE1 | 1.18E−110 | 0.734 | 0.521 | 0.536 | 1.35E−106 |
| MT-ATP8 | 2.30E−107 | 1.048 | 0.565 | 0.555 | 2.62E−103 |
| HMGB1 | 9.17E−107 | 0.506 | 0.529 | 0.696 | 1.04E−102 |
| ST13 | 1.12E−106 | 0.535 | 0.366 | 0.547 | 1.28E−102 |
| HMGN2 | 1.54E−106 | 0.430 | 0.359 | 0.593 | 1.75E−102 |
| EDARADD | 3.66E−105 | 0.462 | 0.192 | 0.357 | 4.17E−101 |
| CD44 | 1.41E−104 | 1.601 | 0.4 | 0.641 | 1.61E−100 |
| UQCR10 | 2.66E−104 | 0.503 | 0.421 | 0.52 | 3.04E−100 |
| KRT8 | 4.06E−104 | 0.806 | 0.405 | 0.44 | 4.63E−100 |
| CHCHD2 | 1.01E−103 | 0.255 | 0.369 | 0.579 | 1.16E−99 |
| OAZ1 | 3.57E−102 | 0.490 | 0.536 | 0.656 | 4.07E−98 |
| RPS4X | 4.92E−102 | 0.278 | 0.671 | 0.795 | 5.60E−98 |
| MT-ND1 | 9.44E−102 | 1.024 | 0.671 | 0.587 | 1.08E−97 |
| EIF1AX | 1.66E−101 | 0.377 | 0.244 | 0.447 | 1.89E−97 |
| COX7B | 9.04E−101 | 0.557 | 0.429 | 0.547 | 1.03E−96 |
| MT-ATP6 | 1.06E−100 | 1.137 | 0.828 | 0.684 | 1.21E−96 |
| TMSB4X | 1.30E−99 | 0.814 | 0.739 | 0.805 | 1.48E−95 |
| SNRPE | 3.77E−99 | 0.625 | 0.51 | 0.607 | 4.30E−95 |
| RBP1 | 3.09E−98 | 0.755 | 0.345 | 0.361 | 3.52E−94 |
| EIF3H | 3.29E−98 | 1.674 | 0.482 | 0.736 | 3.75E−94 |
| CCL28 | 1.43E−97 | 1.532 | 0.273 | 0.001 | 1.63E−93 |
| RPL18A | 2.59E−97 | 0.702 | 0.178 | 0.244 | 2.95E−93 |
| MUC15 | 3.24E−97 | 1.528 | 0.272 | 0.001 | 3.69E−93 |
| ALDOA | 5.38E−97 | 0.564 | 0.493 | 0.608 | 6.13E−93 |
| HLA-C | 6.52E−97 | 0.634 | 0.6 | 0.598 | 7.43E−93 |
| B2M | 3.37E−96 | 0.542 | 0.989 | 0.841 | 3.84E−92 |
| BTF3 | 4.29E−94 | 0.410 | 0.515 | 0.633 | 4.89E−90 |
| SUMO2 | 1.81E−93 | 0.491 | 0.381 | 0.552 | 2.06E−89 |
| KRT18 | 2.58E−93 | 1.318 | 0.309 | 0.013 | 2.94E−89 |
| NME1-NME2 | 5.40E−93 | 0.569 | 0.119 | 0.183 | 6.16E−89 |
| CLDN7 | 2.76E−92 | 1.268 | 0.275 | 0.004 | 3.15E−88 |
| RPL10A | 1.05E−91 | 0.404 | 0.625 | 0.772 | 1.20E−87 |
| RPL17 | 8.71E−91 | 0.874 | 0.217 | 0.185 | 9.92E−87 |
| HLA-A | 1.03E−90 | 0.594 | 0.428 | 0.505 | 1.17E−86 |
| CLDN1 | 1.07E−90 | 1.371 | 0.312 | 0.016 | 1.22E−86 |
| CALML5 | 6.92E−90 | 1.347 | 0.313 | 0.017 | 7.88E−86 |
| SNRPA1 | 2.01E−89 | 0.329 | 0.204 | 0.404 | 2.29E−85 |
| RPS18 | 7.91E−88 | 0.916 | 0.905 | 0.89 | 9.02E−84 |
| EEF1D | 2.43E−87 | 0.291 | 0.319 | 0.517 | 2.77E−83 |
| CARHSP1 | 8.97E−87 | 0.364 | 0.215 | 0.391 | 1.02E−82 |
| TOMM7 | 3.14E−86 | 0.602 | 0.579 | 0.656 | 3.58E−82 |
| SMIM22 | 3.72E−86 | 1.253 | 0.268 | 0.006 | 4.24E−82 |
| MTRNR2L11 | 9.27E−86 | 0.540 | 0.181 | 0.262 | 1.06E−81 |
| RPL36AL | 3.58E−85 | 0.261 | 0.251 | 0.445 | 4.08E−81 |
| CLDN4 | 1.14E−84 | 1.359 | 0.281 | 0.012 | 1.30E−80 |
| TMSB10 | 1.78E−84 | 0.956 | 0.972 | 0.91 | 2.03E−80 |
| PFDN5 | 1.82E−84 | 0.363 | 0.374 | 0.47 | 2.08E−80 |
| SUB1 | 2.31E−84 | 0.378 | 0.547 | 0.669 | 2.63E−80 |
| LTF | 3.77E−84 | 1.535 | 0.236 | 0 | 4.30E−80 |
| UBC | 1.75E−83 | 0.289 | 0.408 | 0.554 | 2.00E−79 |
| ATP5I | 4.34E−83 | 0.294 | 0.304 | 0.461 | 4.94E−79 |
| MTRNR2L13 | 2.18E−82 | 0.360 | 0.08 | 0.193 | 2.48E−78 |
| C9orf16 | 2.20E−81 | 0.251 | 0.131 | 0.324 | 2.51E−77 |
| CLDN3 | 2.72E−81 | 1.298 | 0.281 | 0.014 | 3.10E−77 |
| RPS11 | 3.72E−80 | 0.688 | 0.833 | 0.856 | 4.24E−76 |
| NDUFB4 | 6.71E−80 | 0.343 | 0.306 | 0.453 | 7.65E−76 |
| MT-ND4 | 1.62E−78 | 0.916 | 0.931 | 0.715 | 1.85E−74 |
| RBX1 | 7.95E−78 | 0.513 | 0.375 | 0.471 | 9.06E−74 |
| MAPKAP1 | 1.15E−77 | 1.121 | 0.277 | 0.416 | 1.31E−73 |
| RCN1 | 2.36E−77 | 0.309 | 0.289 | 0.487 | 2.70E−73 |
| TMSB15A | 2.27E−76 | 1.445 | 0.244 | 0.009 | 2.59E−72 |
| IL32 | 3.31E−76 | 0.613 | 0.16 | 0.236 | 3.77E−72 |
| RPS23 | 3.11E−75 | 0.258 | 0.868 | 0.912 | 3.55E−71 |
| DNAJC19 | 8.06E−75 | 0.281 | 0.285 | 0.487 | 9.19E−71 |
| NENF | 6.63E−74 | 0.404 | 0.268 | 0.392 | 7.56E−70 |
| HNRNPC | 2.17E−73 | 0.330 | 0.475 | 0.63 | 2.48E−69 |
| USMG5 | 3.03E−73 | 0.591 | 0.54 | 0.615 | 3.45E−69 |
| BACE2 | 1.36E−71 | 0.288 | 0.117 | 0.242 | 1.55E−67 |
| GUCY1A3 | 4.96E−71 | 1.306 | 0.23 | 0.006 | 5.65E−67 |
| LAD1 | 5.48E−71 | 1.048 | 0.202 | 0 | 6.25E−67 |
| PSME2 | 5.93E−71 | 0.442 | 0.235 | 0.359 | 6.75E−67 |
| SCCPDH | 1.27E−70 | 0.419 | 0.219 | 0.317 | 1.44E−66 |
| COX6B1 | 1.64E−70 | 0.268 | 0.498 | 0.612 | 1.87E−66 |
| SET | 2.96E−70 | 0.310 | 0.534 | 0.722 | 3.37E−66 |
| IFITM3 | 5.57E−70 | 0.269 | 0.408 | 0.571 | 6.35E−66 |
| LINC00152 | 1.27E−69 | 0.709 | 0.256 | 0.289 | 1.45E−65 |
| UTP23 | 1.33E−69 | 1.331 | 0.268 | 0.423 | 1.52E−65 |
| CDC42BPA | 1.80E−69 | 0.323 | 0.17 | 0.29 | 2.05E−65 |
| COX6A1 | 2.48E−68 | 0.276 | 0.498 | 0.653 | 2.83E−64 |
| CD74 | 2.90E−68 | 0.576 | 0.252 | 0.356 | 3.31E−64 |
| MT-ND6 | 3.01E−68 | 0.979 | 0.24 | 0.193 | 3.43E−64 |
| CDK4 | 3.03E−68 | 0.898 | 0.161 | 0.24 | 3.45E−64 |
| S100A13 | 5.66E−68 | 0.259 | 0.302 | 0.505 | 6.45E−64 |
| TIMM13 | 3.08E−67 | 0.278 | 0.232 | 0.387 | 3.51E−63 |
| HLA-B | 4.15E−67 | 0.691 | 0.68 | 0.674 | 4.73E−63 |
| MTRNR2L10 | 4.40E−67 | 0.278 | 0.051 | 0.122 | 5.02E−63 |
| ALDH1A3 | 6.65E−67 | 0.385 | 0.183 | 0.273 | 7.58E−63 |
| PRELID1 | 1.09E−66 | 0.261 | 0.306 | 0.492 | 1.25E−62 |
| NBEAL1 | 4.52E−66 | 0.893 | 0.179 | 0.207 | 5.15E−62 |
| APIP | 5.03E−66 | 0.895 | 0.166 | 0.291 | 5.73E−62 |
| UQCRFS1 | 1.14E−65 | 0.451 | 0.197 | 0.314 | 1.30E−61 |
| COX20 | 1.35E−65 | 0.305 | 0.308 | 0.458 | 1.54E−61 |
| CEACAM6 | 2.49E−65 | 1.210 | 0.199 | 0.002 | 2.83E−61 |
| ERP27 | 1.24E−64 | 1.137 | 0.191 | 0.001 | 1.41E−60 |
| DPM2 | 4.65E−64 | 1.092 | 0.183 | 0.228 | 5.30E−60 |
| DAD1 | 1.74E−63 | 0.285 | 0.284 | 0.439 | 1.99E−59 |
| UBE2T | 2.01E−63 | 1.238 | 0.26 | 0.024 | 2.30E−59 |
| ATF4 | 8.31E−63 | 0.256 | 0.329 | 0.475 | 9.47E−59 |
| PERP | 1.08E−62 | 0.762 | 0.449 | 0.136 | 1.23E−58 |
| IFI6 | 1.90E−62 | 0.684 | 0.749 | 0.604 | 2.16E−58 |
| FTL | 3.92E−62 | 0.334 | 0.655 | 0.767 | 4.46E−58 |
| COX7C | 5.45E−62 | 0.500 | 0.751 | 0.757 | 6.21E−58 |
| MRPL52 | 1.54E−61 | 0.418 | 0.228 | 0.327 | 1.75E−57 |
| PDAP1 | 2.36E−61 | 0.274 | 0.195 | 0.366 | 2.68E−57 |
| COX17 | 4.53E−61 | 0.442 | 0.215 | 0.326 | 5.16E−57 |
| MRPL36 | 4.86E−61 | 0.569 | 0.48 | 0.553 | 5.53E−57 |
| HCFC1R1 | 1.28E−60 | 0.302 | 0.088 | 0.192 | 1.46E−56 |
| NOP10 | 6.76E−60 | 0.278 | 0.253 | 0.421 | 7.70E−56 |
| TMEM258 | 4.35E−59 | 0.282 | 0.321 | 0.459 | 4.95E−55 |
| FAM129B | 1.80E−58 | 0.591 | 0.166 | 0.249 | 2.05E−54 |
| S100P | 6.71E−56 | 1.157 | 0.166 | 0.001 | 7.65E−52 |
| RPS19 | 7.99E−56 | 0.591 | 0.721 | 0.882 | 9.11E−52 |
| PTPRF | 1.54E−55 | 0.256 | 0.324 | 0.465 | 1.75E−51 |
| PDHX | 1.91E−55 | 1.301 | 0.256 | 0.356 | 2.17E−51 |
| JTB | 4.46E−54 | 0.289 | 0.465 | 0.577 | 5.08E−50 |
| NDUFS5 | 4.78E−54 | 0.460 | 0.663 | 0.698 | 5.45E−50 |
| RN7SL1 | 7.64E−54 | 0.325 | 0.115 | 0.262 | 8.71E−50 |
| MT-ND5 | 1.34E−52 | 0.687 | 0.575 | 0.573 | 1.53E−48 |
| C1D | 1.40E−52 | 0.340 | 0.096 | 0.182 | 1.60E−48 |
| RPL27A | 2.34E−52 | 0.316 | 0.757 | 0.86 | 2.67E−48 |
| TMEM176A | 2.41E−52 | 0.999 | 0.159 | 0.001 | 2.75E−48 |
| FAU | 2.46E−52 | 0.282 | 0.705 | 0.751 | 2.81E−48 |
| HORMAD1 | 4.56E−52 | 1.015 | 0.158 | 0.001 | 5.20E−48 |
| EBP | 5.25E−52 | 0.266 | 0.192 | 0.334 | 5.99E−48 |
| MT-TS2 | 8.53E−52 | 0.342 | 0.047 | 0.159 | 9.72E−48 |
| AUP1 | 1.19E−51 | 0.311 | 0.125 | 0.24 | 1.36E−47 |
| COX5B | 1.20E−51 | 0.376 | 0.592 | 0.642 | 1.36E−47 |
| TMEM141 | 1.46E−51 | 0.265 | 0.139 | 0.266 | 1.66E−47 |
| IFI27 | 2.03E−51 | 0.572 | 0.873 | 0.623 | 2.32E−47 |
| RAI14 | 3.00E−51 | 0.460 | 0.256 | 0.303 | 3.41E−47 |
| CDK16 | 3.27E−51 | 0.271 | 0.123 | 0.235 | 3.73E−47 |
| LHCGR | 3.49E−51 | 0.366 | 0.096 | 0.154 | 3.98E−47 |
| PRDX4 | 5.65E−51 | 0.341 | 0.373 | 0.49 | 6.44E−47 |
| TCN1 | 8.27E−51 | 1.325 | 0.148 | 0 | 9.43E−47 |
| NDUFB3 | 1.97E−50 | 0.277 | 0.227 | 0.368 | 2.24E−46 |
| ASS1 | 3.01E−50 | 0.320 | 0.199 | 0.297 | 3.43E−46 |
| APOL6 | 9.46E−50 | 0.645 | 0.269 | 0.291 | 1.08E−45 |
| RNF181 | 1.58E−49 | 0.271 | 0.245 | 0.374 | 1.81E−45 |
| GSN | 6.86E−49 | 0.275 | 0.169 | 0.243 | 7.81E−45 |
| TSFM | 7.85E−49 | 0.854 | 0.144 | 0.109 | 8.95E−45 |
| ACSL3 | 2.06E−48 | 0.926 | 0.292 | 0.059 | 2.35E−44 |
| ARHGDIB | 3.16E−48 | 0.437 | 0.309 | 0.381 | 3.60E−44 |
| LAPTM4B | 6.23E−48 | 0.573 | 0.259 | 0.296 | 7.10E−44 |
| PSMB4 | 8.40E−48 | 0.440 | 0.405 | 0.447 | 9.57E−44 |
| RPL14 | 8.83E−48 | 0.369 | 0.797 | 0.867 | 1.01E−43 |
| PTP4A2 | 1.68E−47 | 0.285 | 0.292 | 0.403 | 1.91E−43 |
| YBX1 | 3.14E−47 | 0.567 | 0.789 | 0.808 | 3.58E−43 |
| STAT1 | 4.25E−47 | 0.332 | 0.281 | 0.346 | 4.85E−43 |
| DDX17 | 5.63E−47 | 0.261 | 0.484 | 0.561 | 6.41E−43 |
| PDZK1IP1 | 2.06E−46 | 0.917 | 0.155 | 0.007 | 2.35E−42 |
| DDT | 3.93E−46 | 0.268 | 0.191 | 0.304 | 4.48E−42 |
| MESP1 | 1.22E−45 | 0.442 | 0.155 | 0.208 | 1.39E−41 |
| ARHGAP29 | 1.23E−45 | 0.732 | 0.14 | 0.001 | 1.41E−41 |
| KIAA0020 | 1.66E−45 | 0.373 | 0.494 | 0.594 | 1.89E−41 |
| PSMB8 | 2.22E−45 | 0.251 | 0.241 | 0.346 | 2.53E−41 |
| ISG15 | 2.58E−45 | 0.258 | 0.47 | 0.484 | 2.95E−41 |
| TXN | 7.47E−45 | 0.399 | 0.718 | 0.756 | 8.52E−41 |
| AARD | 8.63E−45 | 0.422 | 0.208 | 0.319 | 9.83E−41 |
| TMEM9 | 1.15E−44 | 0.320 | 0.243 | 0.342 | 1.31E−40 |
| SAT1 | 1.43E−44 | 0.356 | 0.645 | 0.666 | 1.63E−40 |
| PGBD5 | 1.63E−44 | 0.842 | 0.136 | 0.001 | 1.86E−40 |
| UBE2L6 | 1.74E−43 | 0.328 | 0.158 | 0.22 | 1.99E−39 |
| LAMTOR4 | 2.01E−43 | 0.388 | 0.171 | 0.225 | 2.29E−39 |
| TAOK1 | 2.14E−43 | 3.246 | 0.117 | 0.328 | 2.44E−39 |
| UBBP4 | 3.20E−43 | 0.537 | 0.147 | 0.127 | 3.65E−39 |
| CRABP2 | 8.31E−43 | 0.371 | 0.468 | 0.488 | 9.47E−39 |
| SOX9 | 9.45E−43 | 0.507 | 0.254 | 0.259 | 1.08E−38 |
| IFI44L | 1.68E−42 | 0.451 | 0.21 | 0.221 | 1.91E−38 |
| HINT1 | 2.55E−42 | 0.323 | 0.654 | 0.701 | 2.91E−38 |
| XBP1 | 2.62E−42 | 0.587 | 0.237 | 0.37 | 2.98E−38 |
| CDK2AP1 | 3.41E−42 | 0.299 | 0.085 | 0.152 | 3.89E−38 |
| SDC4 | 4.66E−42 | 0.717 | 0.159 | 0.009 | 5.31E−38 |
| RPL13 | 5.40E−42 | 0.366 | 0.966 | 0.854 | 6.16E−38 |
| C17orf89 | 5.43E−42 | 0.349 | 0.191 | 0.282 | 6.19E−38 |
| EDN1 | 2.14E−41 | 0.723 | 0.162 | 0.014 | 2.44E−37 |
| MT-TP | 2.16E−41 | 0.980 | 0.088 | 0.149 | 2.47E−37 |
| GNL3 | 3.13E−41 | 0.280 | 0.349 | 0.452 | 3.56E−37 |
| RPA3 | 3.59E−41 | 0.270 | 0.206 | 0.316 | 4.10E−37 |
| PTMA | 4.83E−41 | 0.478 | 0.9 | 0.911 | 5.51E−37 |
| VWA1 | 9.64E−41 | 0.312 | 0.131 | 0.2 | 1.10E−36 |
| HAPLN3 | 1.76E−40 | 0.841 | 0.133 | 0.003 | 2.00E−36 |
| MT-RNR1 | 2.97E−40 | 0.705 | 0.97 | 0.895 | 3.38E−36 |
| TRIM44 | 3.15E−40 | 0.380 | 0.218 | 0.298 | 3.59E−36 |
| MIR4435-1HG | 5.25E−40 | 0.494 | 0.128 | 0.161 | 5.98E−36 |
| EXOSC4 | 6.51E−40 | 0.255 | 0.104 | 0.191 | 7.42E−36 |
| C5orf46 | 8.74E−40 | 0.830 | 0.117 | 0 | 9.96E−36 |
| RAB25 | 8.91E−40 | 0.602 | 0.133 | 0.004 | 1.02E−35 |
| RARRES3 | 1.54E−39 | 0.684 | 0.169 | 0.132 | 1.76E−35 |
| NDUFA6 | 1.60E−39 | 0.350 | 0.58 | 0.609 | 1.83E−35 |
| RPL24 | 2.00E−39 | 0.460 | 0.796 | 0.81 | 2.28E−35 |
| IFITM2 | 2.30E−39 | 0.340 | 0.117 | 0.172 | 2.62E−35 |
| SULT1B1 | 4.58E−39 | 0.863 | 0.118 | 0.001 | 5.22E−35 |
| LGALS1 | 5.27E−39 | 0.269 | 0.578 | 0.678 | 6.00E−35 |
| ANKRD30A | 1.22E−38 | 1.739 | 0.114 | 0 | 1.39E−34 |
| CCL5 | 2.53E−38 | 0.584 | 0.158 | 0.025 | 2.88E−34 |
| MGST3 | 3.02E−38 | 0.347 | 0.514 | 0.581 | 3.44E−34 |
| IFIH1 | 5.22E−38 | 0.397 | 0.347 | 0.381 | 5.95E−34 |
| CAMSAP2 | 6.58E−38 | 0.310 | 0.186 | 0.274 | 7.50E−34 |
| ESRP1 | 8.72E−38 | 0.548 | 0.112 | 0 | 9.93E−34 |
| SOD1 | 9.82E−38 | 0.265 | 0.564 | 0.639 | 1.12E−33 |
| SFN | 1.68E−37 | 0.592 | 0.111 | 0 | 1.91E−33 |
| SEPP1 | 3.14E−37 | 1.304 | 0.215 | 0.26 | 3.58E−33 |
| ATP6V1E1 | 1.24E−36 | 0.319 | 0.183 | 0.293 | 1.41E−32 |
| SIX1 | 2.30E−36 | 0.495 | 0.108 | 0 | 2.62E−32 |
| PYCR1 | 2.47E−36 | 0.561 | 0.13 | 0.005 | 2.81E−32 |
| CXCL17 | 4.08E−36 | 0.744 | 0.113 | 0.001 | 4.65E−32 |
| ACOT9 | 6.94E−36 | 0.335 | 0.16 | 0.231 | 7.91E−32 |
| NES | 7.64E−36 | 0.408 | 0.088 | 0.102 | 8.70E−32 |
| SECTM1 | 8.50E−36 | 0.711 | 0.106 | 0 | 9.68E−32 |
| GRHL2 | 8.50E−36 | 0.569 | 0.106 | 0 | 9.68E−32 |
| GSTK1 | 2.45E−35 | 0.265 | 0.15 | 0.246 | 2.79E−31 |
| MRPS18C | 3.42E−35 | 0.302 | 0.303 | 0.394 | 3.90E−31 |
| RPL37A | 8.34E−35 | 0.481 | 0.848 | 0.911 | 9.51E−31 |
| PRC1 | 1.12E−34 | 0.459 | 0.296 | 0.292 | 1.28E−30 |
| RTP4 | 1.21E−34 | 0.382 | 0.166 | 0.188 | 1.38E−30 |
| GBP2 | 1.80E−34 | 0.827 | 0.156 | 0.148 | 2.06E−30 |
| PRSS8 | 2.11E−34 | 0.572 | 0.125 | 0.005 | 2.40E−30 |
| EIF2S3L | 2.86E−34 | 0.300 | 0.087 | 0.137 | 3.26E−30 |
| GBP1 | 7.40E−34 | 0.575 | 0.203 | 0.212 | 8.43E−30 |
| H2AFZ | 1.01E−33 | 0.373 | 0.607 | 0.6 | 1.15E−29 |
| GTF2H5 | 5.18E−33 | 0.251 | 0.15 | 0.237 | 5.90E−29 |
| STK25 | 5.29E−33 | 0.255 | 0.077 | 0.142 | 6.03E−29 |
| FGD5-AS1 | 1.13E−32 | 1.516 | 0.133 | 0.304 | 1.28E−28 |
| DUSP18 | 1.73E−32 | 0.317 | 0.092 | 0.115 | 1.97E−28 |
| PSMB9 | 2.89E−32 | 0.278 | 0.134 | 0.203 | 3.29E−28 |
| CACYBP | 2.99E−32 | 0.311 | 0.588 | 0.691 | 3.41E−28 |
| NPNT | 3.26E−32 | 0.618 | 0.102 | 0.001 | 3.72E−28 |
| RPS12 | 4.08E−32 | 0.392 | 0.979 | 0.978 | 4.65E−28 |
| INSIG1 | 4.44E−32 | 0.517 | 0.178 | 0.279 | 5.06E−28 |
| CTD-2090I13.1 | 6.22E−32 | 0.260 | 0.069 | 0.102 | 7.09E−28 |
| RPS24 | 1.03E−31 | 0.361 | 0.888 | 0.919 | 1.17E−27 |
| SDC1 | 1.09E−31 | 0.718 | 0.146 | 0.016 | 1.24E−27 |
| IGF1R | 1.28E−31 | 1.519 | 0.167 | 0.151 | 1.46E−27 |
| XAF1 | 1.98E−31 | 0.714 | 0.234 | 0.16 | 2.26E−27 |
| NACA2 | 2.51E−31 | 0.258 | 0.113 | 0.163 | 2.86E−27 |
| ANKRD36C | 2.98E−31 | 0.376 | 0.221 | 0.259 | 3.39E−27 |
| TSIX | 8.97E−31 | 0.520 | 0.129 | 0.082 | 1.02E−26 |
| ATP8B1 | 1.51E−30 | 0.512 | 0.119 | 0.007 | 1.72E−26 |
| IFIT3 | 1.75E−30 | 0.488 | 0.219 | 0.205 | 1.99E−26 |
| SRSF7 | 1.99E−30 | 1.595 | 0.252 | 0.41 | 2.27E−26 |
| LINC00511 | 1.18E−29 | 0.282 | 0.084 | 0.109 | 1.34E−25 |
| SEC31B | 1.27E−29 | 0.274 | 0.079 | 0.117 | 1.45E−25 |
| PARP14 | 1.34E−29 | 0.463 | 0.24 | 0.253 | 1.53E−25 |
| CDK11B | 1.37E−29 | 0.261 | 0.069 | 0.121 | 1.56E−25 |
| TFAP2A | 1.43E−29 | 0.504 | 0.117 | 0.008 | 1.63E−25 |
| TSPAN13 | 3.45E−28 | 0.629 | 0.225 | 0.06 | 3.94E−24 |
| MT-CYB | 4.13E−28 | 0.258 | 0.856 | 0.695 | 4.71E−24 |
| CP | 4.68E−28 | 0.948 | 0.562 | 0.525 | 5.34E−24 |
| GPR160 | 8.09E−28 | 0.564 | 0.152 | 0.023 | 9.22E−24 |
| CD2AP | 8.33E−28 | 0.382 | 0.242 | 0.307 | 9.49E−24 |
| CCDC34 | 2.96E−27 | 0.268 | 0.154 | 0.234 | 3.37E−23 |
| RAB27B | 3.88E−27 | 0.478 | 0.103 | 0.005 | 4.42E−23 |
| RPS29 | 4.15E−27 | 0.504 | 0.942 | 0.938 | 4.73E−23 |
| TPRKB | 1.53E−26 | 0.253 | 0.231 | 0.318 | 1.74E−22 |
| CD151 | 1.89E−26 | 0.618 | 0.175 | 0.038 | 2.15E−22 |
| RPL35 | 2.83E−26 | 0.397 | 0.914 | 0.891 | 3.23E−22 |
| AP000350.4 | 3.10E−26 | 0.607 | 0.118 | 0.063 | 3.53E−22 |
| XRCC6BP1 | 4.76E−26 | 0.644 | 0.112 | 0.01 | 5.43E−22 |
| MARVELD2 | 1.51E−24 | 0.465 | 0.109 | 0.01 | 1.72E−20 |
| IFIT2 | 2.84E−24 | 0.411 | 0.146 | 0.109 | 3.24E−20 |
| HLA-DRA | 3.24E−24 | 0.403 | 0.196 | 0.179 | 3.70E−20 |
| ACBD5 | 4.80E−24 | 0.311 | 0.162 | 0.203 | 5.47E−20 |
| MT-ND2 | 7.40E−24 | 0.304 | 0.886 | 0.734 | 8.43E−20 |
| HINT2 | 9.90E−24 | 0.319 | 0.138 | 0.193 | 1.13E−19 |
| ATP5E | 2.18E−23 | 0.338 | 0.792 | 0.781 | 2.48E−19 |
| APOBEC3B | 2.62E−23 | 0.615 | 0.104 | 0.011 | 2.98E−19 |
| CDH1 | 3.69E−23 | 0.514 | 0.11 | 0.012 | 4.21E−19 |
| EPSTI1 | 3.41E−22 | 0.336 | 0.112 | 0.111 | 3.88E−18 |
| SLC35B3 | 7.44E−22 | 0.265 | 0.077 | 0.123 | 8.48E−18 |
| BSPRY | 3.60E−21 | 0.580 | 0.116 | 0.016 | 4.10E−17 |
| RP11-304L19.5 | 7.32E−21 | 0.452 | 0.128 | 0.023 | 8.34E−17 |
| DPP7 | 5.01E−20 | 0.255 | 0.082 | 0.111 | 5.71E−16 |
| ISG20 | 7.26E−20 | 0.341 | 0.096 | 0.131 | 8.28E−16 |
| CENPN | 7.44E−20 | 0.419 | 0.105 | 0.016 | 8.48E−16 |
| SAMD9L | 1.16E−19 | 0.564 | 0.171 | 0.142 | 1.33E−15 |
| CRELD2 | 1.12E−18 | 0.438 | 0.105 | 0.016 | 1.28E−14 |
| OPN3 | 2.09E−18 | 0.589 | 0.13 | 0.033 | 2.38E−14 |
| MT2A | 3.13E−18 | 0.630 | 0.14 | 0.11 | 3.57E−14 |
| B3GNT2 | 3.50E−18 | 0.456 | 0.109 | 0.018 | 3.98E−14 |
| PTTG1 | 5.38E−18 | 0.570 | 0.149 | 0.044 | 6.13E−14 |
| MLK4 | 6.00E−18 | 0.579 | 0.101 | 0.017 | 6.84E−14 |
| HLA-DPA1 | 1.96E−17 | 0.291 | 0.097 | 0.119 | 2.24E−13 |
| RPS13 | 3.05E−16 | 0.283 | 0.894 | 0.884 | 3.47E−12 |
| LSR | 3.17E−15 | 0.454 | 0.116 | 0.027 | 3.61E−11 |
| SSR4 | 1.02E−14 | 0.306 | 0.78 | 0.674 | 1.16E−10 |
| CD59 | 3.57E−14 | 0.317 | 0.738 | 0.707 | 4.07E−10 |
| GALNT3 | 5.65E−14 | 0.420 | 0.15 | 0.05 | 6.44E−10 |
| PKIB | 2.16E−13 | 0.354 | 0.158 | 0.062 | 2.47E−09 |
| TRIP13 | 3.32E−13 | 0.436 | 0.113 | 0.031 | 3.79E−09 |
| SAMD9 | 4.54E−13 | 0.350 | 0.16 | 0.132 | 5.18E−09 |
| GATA3 | 1.11E−12 | 0.308 | 0.117 | 0.132 | 1.27E−08 |
| PLA2G16 | 2.11E−12 | 0.319 | 0.121 | 0.08 | 2.41E−08 |
| ATL2 | 2.14E−12 | 0.450 | 0.111 | 0.031 | 2.44E−08 |
| SLC39A6 | 6.17E−11 | 0.466 | 0.125 | 0.044 | 7.04E−07 |
| EBPL | 7.78E−11 | 0.364 | 0.116 | 0.038 | 8.86E−07 |
| MZT1 | 7.57E−10 | 0.341 | 0.113 | 0.04 | 8.63E−06 |
| RBCK1 | 5.79E−09 | 0.302 | 0.103 | 0.09 | 6.59E−05 |
| FNBP1L | 1.73E−08 | 0.322 | 0.119 | 0.092 | 1.97E−04 |

CSF_full.r2.setRvNR.tumor.pre.bimod.markers.up.1

| | | | | | |
|---|---|---|---|---|---|
| RPL19 | 2.38E−269 | 4.394 | 0.887 | 0.787 | 2.72E−265 |
| SCGB2A2 | 9.77E−250 | 5.941 | 0.819 | 0.001 | 1.11E−245 |
| MGP | 1.51E−220 | 4.926 | 0.77 | 0.004 | 1.72E−216 |
| XBP1 | 4.30E−189 | 2.583 | 0.824 | 0.139 | 4.90E−185 |
| EIF3H | 1.11E−171 | 2.965 | 0.921 | 0.513 | 1.26E−167 |
| RPL12 | 6.47E−165 | 3.147 | 0.941 | 0.788 | 7.38E−161 |
| RPL37 | 2.63E−159 | 1.639 | 0.968 | 0.67 | 3.00E−155 |
| CLDN4 | 3.05E−157 | 2.338 | 0.621 | 0.006 | 3.48E−153 |
| RPS24 | 1.71E−149 | 1.911 | 0.983 | 0.873 | 1.95E−145 |
| KRT19 | 4.52E−148 | 2.310 | 0.586 | 0.003 | 5.15E−144 |
| SCGB1D2 | 1.12E−147 | 3.253 | 0.577 | 0.001 | 1.27E−143 |
| KRT18 | 1.47E−138 | 1.982 | 0.571 | 0.006 | 1.68E−134 |
| TACSTD2 | 4.58E−132 | 2.158 | 0.544 | 0.004 | 5.22E−128 |
| DCD | 1.61E−125 | 4.958 | 0.512 | 0.001 | 1.84E−121 |
| FOS | 5.34E−118 | 1.779 | 0.772 | 0.171 | 6.09E−114 |
| MIEN1 | 4.74E−115 | 2.624 | 0.544 | 0.124 | 5.40E−111 |
| MGST1 | 7.37E−106 | 1.606 | 0.651 | 0.091 | 8.40E−102 |
| CLDN3 | 5.81E−105 | 1.615 | 0.458 | 0.004 | 6.62E−101 |
| RAB11FIP1 | 1.85E−103 | 2.263 | 0.58 | 0.095 | 2.11E−99 |
| RPS2 | 5.77E−102 | 1.395 | 0.867 | 0.461 | 6.57E−98 |
| NUPR1 | 4.49E−101 | 1.962 | 0.605 | 0.075 | 5.12E−97 |
| TFAP2A | 4.41E−100 | 1.505 | 0.448 | 0.006 | 5.03E−96 |
| RPS27A | 5.78E−99 | 1.129 | 0.954 | 0.741 | 6.59E−95 |
| MLPH | 1.26E−98 | 1.416 | 0.431 | 0.003 | 1.43E−94 |
| CYP4Z1 | 9.09E−98 | 1.784 | 0.412 | 0 | 1.04E−93 |
| ERBB2 | 8.94E−97 | 2.210 | 0.501 | 0.072 | 1.02E−92 |
| ANKRD30A | 4.59E−95 | 4.078 | 0.403 | 0 | 5.24E−91 |
| RPL26 | 5.76E−95 | 1.342 | 0.951 | 0.623 | 6.56E−91 |
| ANKRD30B | 6.19E−91 | 1.989 | 0.401 | 0.003 | 7.06E−87 |
| AGR2 | 1.02E−88 | 1.707 | 0.388 | 0.001 | 1.16E−84 |
| GATA3 | 2.71E−88 | 1.799 | 0.491 | 0.037 | 3.09E−84 |
| RPL37A | 3.82E−88 | 0.962 | 0.979 | 0.822 | 4.36E−84 |
| ADIRF | 2.72E−87 | 1.590 | 0.391 | 0.003 | 3.10E−83 |
| EPCAM | 3.19E−87 | 1.285 | 0.39 | 0.003 | 3.63E−83 |
| PLP1 | 1.12E−86 | 1.963 | 0.372 | 0 | 1.28E−82 |
| CAMK2N1 | 3.04E−86 | 1.414 | 0.371 | 0 | 3.47E−82 |
| PNMT | 4.30E−86 | 1.591 | 0.379 | 0.001 | 4.90E−82 |
| RPS21 | 5.97E−86 | 1.143 | 0.983 | 0.895 | 6.80E−82 |
| ESRP1 | 6.06E−85 | 1.144 | 0.366 | 0 | 6.90E−81 |
| GLYATL2 | 4.41E−84 | 2.977 | 0.363 | 0 | 5.03E−80 |
| CD44 | 3.19E−82 | 2.283 | 0.515 | 0.443 | 3.64E−78 |
| RPS12 | 5.96E−81 | 0.735 | 0.994 | 0.939 | 6.79E−77 |
| UTP23 | 2.56E−80 | 2.306 | 0.674 | 0.281 | 2.91E−76 |
| CDH1 | 6.05E−80 | 1.370 | 0.365 | 0.003 | 6.90E−76 |
| MDK | 8.24E−80 | 1.312 | 0.347 | 0 | 9.39E−76 |
| RPL11 | 2.35E−76 | 0.905 | 0.978 | 0.822 | 2.68E−72 |
| GPC3 | 3.45E−75 | 1.459 | 0.33 | 0 | 3.93E−71 |
| RPL32 | 6.79E−75 | 1.172 | 0.967 | 0.873 | 7.74E−71 |
| LRRC26 | 4.02E−74 | 1.189 | 0.342 | 0.003 | 4.58E−70 |
| MED1 | 1.86E−72 | 1.565 | 0.466 | 0.088 | 2.12E−68 |
| PGAP3 | 5.57E−71 | 0.966 | 0.32 | 0.001 | 6.35E−67 |
| RPS15A | 1.65E−70 | 1.049 | 0.94 | 0.721 | 1.88E−66 |
| NBEAL1 | 5.89E−70 | 2.357 | 0.601 | 0.222 | 6.71E−66 |
| PLA2G16 | 2.88E−69 | 1.235 | 0.361 | 0.013 | 3.29E−65 |
| CLDN7 | 1.33E−68 | 0.862 | 0.304 | 0 | 1.51E−64 |
| STAC2 | 3.38E−68 | 1.679 | 0.303 | 0 | 3.86E−64 |
| SNHG8 | 8.46E−68 | 1.399 | 0.658 | 0.322 | 9.64E−64 |
| SEPP1 | 2.43E−67 | 1.881 | 0.512 | 0.092 | 2.77E−63 |
| CTTN | 4.28E−67 | 1.605 | 0.468 | 0.066 | 4.88E−63 |
| RNU6-6P | 1.44E−66 | 1.631 | 0.401 | 0.034 | 1.65E−62 |
| GRB7 | 1.90E−66 | 1.176 | 0.361 | 0.018 | 2.16E−62 |
| MT-TE | 1.10E−65 | 1.390 | 0.312 | 0.004 | 1.26E−61 |
| S100A14 | 3.29E−65 | 1.248 | 0.426 | 0.047 | 3.75E−61 |
| CYR61 | 4.00E−65 | 1.403 | 0.3 | 0.001 | 4.56E−61 |
| SPDEF | 4.92E−65 | 0.991 | 0.345 | 0.013 | 5.61E−61 |
| SMIM14 | 5.75E−65 | 1.276 | 0.461 | 0.066 | 6.55E−61 |
| JUNB | 2.52E−64 | 1.417 | 0.645 | 0.243 | 2.88E−60 |
| RP11-304L19.5 | 3.44E−63 | 1.199 | 0.48 | 0.085 | 3.92E−59 |
| ALDH3B2 | 2.22E−61 | 0.868 | 0.276 | 0 | 2.53E−57 |
| SERINC2 | 1.37E−60 | 0.830 | 0.273 | 0 | 1.56E−56 |
| LDHA | 2.17E−60 | 1.561 | 0.666 | 0.501 | 2.48E−56 |
| CA12 | 3.12E−60 | 0.921 | 0.281 | 0.001 | 3.56E−56 |
| FOXA1 | 1.20E−58 | 0.950 | 0.288 | 0.006 | 1.37E−54 |
| KRT8 | 1.77E−57 | 0.625 | 0.512 | 0.13 | 2.01E−53 |
| SPINK8 | 1.89E−57 | 0.900 | 0.26 | 0 | 2.16E−53 |
| SDC4 | 2.17E−57 | 0.907 | 0.269 | 0.001 | 2.47E−53 |
| SERHL2 | 4.65E−57 | 0.859 | 0.258 | 0 | 5.30E−53 |
| STARD3 | 7.14E−57 | 1.063 | 0.39 | 0.047 | 8.13E−53 |
| TMEM254 | 3.42E−56 | 0.989 | 0.338 | 0.023 | 3.90E−52 |
| DSP | 5.19E−56 | 0.916 | 0.656 | 0.238 | 5.91E−52 |
| S100A8 | 1.70E−55 | 1.388 | 0.458 | 0.083 | 1.94E−51 |
| RPL34 | 2.37E−55 | 0.870 | 0.943 | 0.753 | 2.70E−51 |
| NQO1 | 2.62E−55 | 1.057 | 0.458 | 0.083 | 2.98E−51 |
| C8orf4 | 9.90E−55 | 1.362 | 0.249 | 0 | 1.13E−50 |
| AZGP1 | 3.50E−54 | 0.877 | 0.683 | 0.323 | 3.99E−50 |
| HSPA1A | 5.18E−53 | 1.486 | 0.51 | 0.148 | 5.90E−49 |
| AGR3 | 8.33E−53 | 1.129 | 0.241 | 0 | 9.50E−49 |
| UPF2 | 1.08E−52 | 0.954 | 0.429 | 0.075 | 1.23E−48 |
| RPLP0 | 1.70E−52 | 0.895 | 0.932 | 0.76 | 1.94E−48 |
| RPS15 | 2.50E−52 | 0.891 | 0.853 | 0.535 | 2.85E−48 |
| FOSB | 7.66E−51 | 1.299 | 0.336 | 0.031 | 8.73E−47 |
| RPS19 | 1.34E−50 | 0.481 | 0.987 | 0.885 | 1.53E−46 |
| IER2 | 1.05E−49 | 1.353 | 0.599 | 0.266 | 1.20E−45 |
| MAL2 | 1.38E−49 | 0.447 | 0.442 | 0.123 | 1.57E−45 |
| ANO1 | 1.51E−49 | 0.736 | 0.238 | 0.001 | 1.72E−45 |
| ENPP1 | 6.36E−49 | 0.925 | 0.292 | 0.022 | 7.25E−45 |
| CLCA2 | 3.06E−48 | 1.025 | 0.222 | 0 | 3.49E−44 |
| EEF1D | 4.78E−48 | 0.977 | 0.719 | 0.427 | 5.45E−44 |
| RPS11 | 1.28E−47 | 0.553 | 0.964 | 0.766 | 1.46E−43 |
| IGF1R | 1.61E−47 | 2.579 | 0.314 | 0.056 | 1.84E−43 |
| AQP5 | 1.74E−47 | 0.894 | 0.219 | 0 | 1.98E−43 |
| NUDT8 | 2.07E−47 | 0.808 | 0.228 | 0.001 | 2.36E−43 |
| CDK12 | 3.92E−47 | 1.094 | 0.426 | 0.088 | 4.47E−43 |
| CYB5A | 8.64E−47 | 0.961 | 0.399 | 0.07 | 9.85E−43 |
| IRX2 | 9.80E−47 | 0.777 | 0.216 | 0 | 1.12E−42 |
| TSPAN15 | 2.55E−46 | 0.626 | 0.255 | 0.013 | 2.90E−42 |
| MAPKAP1 | 6.01E−46 | 1.712 | 0.485 | 0.154 | 6.84E−42 |
| METRN | 6.28E−46 | 0.800 | 0.29 | 0.022 | 7.16E−42 |
| DUSP4 | 2.99E−45 | 1.229 | 0.452 | 0.111 | 3.40E−41 |
| TPD52L1 | 3.36E−45 | 0.907 | 0.33 | 0.045 | 3.82E−41 |
| FXYD3 | 3.99E−45 | 0.722 | 0.236 | 0.006 | 4.55E−41 |
| PYCR1 | 5.27E−45 | 0.778 | 0.261 | 0.013 | 6.00E−41 |
| VTRNA1-2 | 7.26E−45 | 0.719 | 0.208 | 0 | 8.27E−41 |
| GALNT7 | 9.99E−45 | 0.805 | 0.333 | 0.044 | 1.14E−40 |
| RP11-206M11.7 | 1.71E−44 | 1.391 | 0.206 | 0 | 1.95E−40 |
| RPS6 | 2.55E−44 | 0.376 | 0.994 | 0.966 | 2.91E−40 |
| PTPRF | 3.33E−44 | 0.590 | 0.426 | 0.115 | 3.80E−40 |
| MAFB | 4.48E−44 | 0.694 | 0.238 | 0.012 | 5.11E−40 |
| FNBP1L | 5.77E−44 | 1.014 | 0.279 | 0.022 | 6.58E−40 |
| SLC40A1 | 9.81E−44 | 0.989 | 0.296 | 0.031 | 1.12E−39 |
| NPDC1 | 1.29E−43 | 0.899 | 0.344 | 0.05 | 1.47E−39 |
| KAT6B | 2.52E−43 | 0.981 | 0.38 | 0.07 | 2.87E−39 |
| NTPCR | 2.52E−43 | 4.688 | 0.379 | 0.067 | 2.87E−39 |
| TRIQK | 4.38E−43 | 0.756 | 0.217 | 0.003 | 4.99E−39 |
| TOB1 | 4.39E−43 | 0.841 | 0.547 | 0.18 | 5.01E−39 |
| THRSP | 1.22E−42 | 0.712 | 0.198 | 0 | 1.39E−38 |
| SCGB2A1 | 1.22E−42 | 1.064 | 0.198 | 0 | 1.39E−38 |
| KIAA1324 | 1.31E−42 | 0.806 | 0.219 | 0.004 | 1.50E−38 |
| EDN1 | 3.52E−42 | 1.316 | 0.204 | 0.001 | 4.02E−38 |
| RASD1 | 5.08E−42 | 0.866 | 0.233 | 0.009 | 5.79E−38 |
| DHRS2 | 6.69E−42 | 0.549 | 0.195 | 0 | 7.62E−38 |
| ORMDL3 | 7.65E−42 | 0.833 | 0.36 | 0.066 | 8.71E−38 |
| RPS10 | 1.81E−41 | 0.740 | 0.731 | 0.371 | 2.06E−37 |
| RPS14 | 3.23E−41 | 0.860 | 0.956 | 0.865 | 3.68E−37 |
| RNA5SP149 | 3.64E−41 | 0.655 | 0.192 | 0 | 4.15E−37 |
| CNN3 | 3.84E−41 | 0.535 | 0.36 | 0.079 | 4.38E−37 |
| FBXL20 | 6.09E−41 | 0.536 | 0.222 | 0.009 | 6.95E−37 |
| GRINA | 1.63E−40 | 0.648 | 0.347 | 0.063 | 1.85E−36 |
| ALDOA | 3.48E−40 | 0.898 | 0.735 | 0.409 | 3.97E−36 |
| HSPB1 | 8.08E−40 | 0.822 | 0.518 | 0.175 | 9.21E−36 |
| CSNK1A1 | 9.83E−40 | 0.823 | 0.523 | 0.183 | 1.12E−35 |
| VDAC2 | 2.48E−39 | 0.966 | 0.494 | 0.19 | 2.83E−35 |
| NFIA | 4.58E−39 | 0.655 | 0.331 | 0.06 | 5.22E−35 |
| RPS28 | 4.66E−39 | 0.599 | 0.873 | 0.576 | 5.31E−35 |
| GRHL2 | 5.72E−39 | 0.523 | 0.182 | 0 | 6.52E−35 |
| RPL23A | 7.93E−39 | 0.714 | 0.823 | 0.499 | 9.03E−35 |
| NEAT1 | 1.15E−38 | 0.837 | 0.61 | 0.256 | 1.31E−34 |
| VTRNA1-3 | 3.51E−38 | 1.204 | 0.309 | 0.045 | 4.00E−34 |
| DPM2 | 3.97E−38 | 1.262 | 0.399 | 0.101 | 4.52E−34 |
| CCND1 | 4.75E−38 | 1.266 | 0.474 | 0.165 | 5.42E−34 |
| TSPAN1 | 8.06E−38 | 0.681 | 0.204 | 0.006 | 9.19E−34 |
| GBP2 | 1.05E−37 | 1.392 | 0.288 | 0.037 | 1.20E−33 |
| SMIM22 | 1.63E−37 | 0.597 | 0.176 | 0 | 1.86E−33 |
| ERGIC1 | 1.70E−37 | 0.873 | 0.382 | 0.086 | 1.94E−33 |
| AR | 5.25E−37 | 0.696 | 0.181 | 0.001 | 5.99E−33 |
| FGD5 | 8.62E−37 | 0.529 | 0.173 | 0 | 9.83E−33 |
| ZNF704 | 1.93E−36 | 0.666 | 0.206 | 0.009 | 2.20E−32 |
| NPNT | 1.98E−36 | 0.487 | 0.171 | 0 | 2.26E−32 |
| MT-TV | 2.03E−36 | 1.241 | 0.471 | 0.158 | 2.32E−32 |
| CNTNAP2 | 3.02E−36 | 0.472 | 0.19 | 0.006 | 3.44E−32 |
| P4HA1 | 4.09E−36 | 0.821 | 0.338 | 0.064 | 4.66E−32 |
| GPRC5A | 4.55E−36 | 0.529 | 0.17 | 0 | 5.18E−32 |
| KIAA1244 | 5.88E−36 | 0.655 | 0.277 | 0.042 | 6.70E−32 |
| MALAT1 | 7.23E−36 | 1.495 | 0.989 | 0.928 | 8.24E−32 |
| CCDC6 | 7.63E−36 | 0.747 | 0.46 | 0.158 | 8.70E−32 |
| HES1 | 1.21E−35 | 1.042 | 0.613 | 0.311 | 1.38E−31 |
| GOLGB1 | 1.30E−35 | 1.069 | 0.55 | 0.216 | 1.48E−31 |
| ESR1 | 1.95E−35 | 1.478 | 0.206 | 0.013 | 2.22E−31 |
| RPL5 | 2.05E−35 | 0.383 | 0.932 | 0.713 | 2.34E−31 |
| RAB27B | 2.54E−35 | 0.599 | 0.211 | 0.01 | 2.89E−31 |
| GCAT | 5.46E−35 | 0.464 | 0.165 | 0 | 6.23E−31 |
| TMEM256 | 6.72E−35 | 0.821 | 0.325 | 0.069 | 7.66E−31 |
| BTF3 | 6.90E−35 | 0.544 | 0.7 | 0.377 | 7.87E−31 |
| SUSD2 | 9.63E−35 | 0.481 | 0.174 | 0.001 | 1.10E−30 |
| SLC2A10 | 1.25E−34 | 0.516 | 0.163 | 0 | 1.42E−30 |
| ABCC11 | 1.25E−34 | 0.535 | 0.163 | 0 | 1.42E−30 |
| C3orf14 | 1.37E−34 | 0.619 | 0.282 | 0.042 | 1.56E−30 |
| GSTM3 | 1.68E−34 | 0.477 | 0.19 | 0.007 | 1.92E−30 |
| RPS25 | 2.06E−34 | 0.512 | 0.845 | 0.542 | 2.35E−30 |
| IRX3 | 3.27E−34 | 0.566 | 0.334 | 0.08 | 3.73E−30 |
| FAM210B | 3.50E−34 | 0.839 | 0.366 | 0.088 | 3.99E−30 |
| CASC3 | 5.52E−34 | 0.823 | 0.353 | 0.082 | 6.29E−30 |
| SIX1 | 6.50E−34 | 0.747 | 0.16 | 0 | 7.40E−30 |
| PCLO | 6.50E−34 | 0.612 | 0.16 | 0 | 7.40E−30 |
| MAGEA3 | 6.50E−34 | 0.378 | 0.16 | 0 | 7.40E−30 |
| HK1 | 8.18E−34 | 0.655 | 0.311 | 0.057 | 9.32E−30 |
| S100A9 | 1.60E−33 | 0.909 | 0.439 | 0.146 | 1.83E−29 |
| PPFIA1 | 1.85E−33 | 0.899 | 0.415 | 0.126 | 2.11E−29 |
| RP11-369C8.1 | 3.37E−33 | 0.424 | 0.157 | 0 | 3.84E−29 |
| VTCN1 | 7.65E−33 | 0.707 | 0.155 | 0 | 8.72E−29 |
| CCDC57 | 9.06E−33 | 0.453 | 0.236 | 0.028 | 1.03E−28 |
| GSTO2 | 1.09E−32 | 0.491 | 0.163 | 0.001 | 1.25E−28 |
| H2AFJ | 1.18E−32 | 0.861 | 0.482 | 0.183 | 1.35E−28 |
| RP11-690G19.3 | 1.23E−32 | 0.322 | 0.16 | 0.001 | 1.40E−28 |
| CRABP2 | 1.42E−32 | 0.453 | 0.431 | 0.17 | 1.62E−28 |
| KCNMA1 | 1.70E−32 | 0.814 | 0.249 | 0.029 | 1.94E−28 |
| MT2A | 2.87E−32 | 0.871 | 0.49 | 0.18 | 3.27E−28 |
| TSFM | 3.77E−32 | 1.346 | 0.258 | 0.047 | 4.30E−28 |
| TSKU | 3.94E−32 | 0.373 | 0.152 | 0 | 4.49E−28 |
| RPL7A | 5.53E−32 | 0.733 | 0.802 | 0.573 | 6.30E−28 |
| MYL12B | 6.36E−32 | 0.890 | 0.683 | 0.487 | 7.25E−28 |
| COMTD1 | 6.61E−32 | 0.785 | 0.301 | 0.057 | 7.53E−28 |
| RHOD | 6.91E−32 | 0.276 | 0.176 | 0.012 | 7.88E−28 |
| RHOBTB1 | 1.01E−31 | 0.718 | 0.236 | 0.026 | 1.15E−27 |
| VAV3 | 1.22E−31 | 0.547 | 0.217 | 0.02 | 1.39E−27 |
| EGR1 | 1.25E−31 | 0.843 | 0.59 | 0.273 | 1.42E−27 |
| MT-TF | 1.72E−31 | 0.475 | 0.187 | 0.013 | 1.96E−27 |
| DUSP1 | 2.91E−31 | 0.786 | 0.39 | 0.111 | 3.32E−27 |
| PRLR | 4.43E−31 | 0.395 | 0.249 | 0.042 | 5.05E−27 |
| HSPA1B | 5.09E−31 | 0.920 | 0.407 | 0.136 | 5.81E−27 |
| MT-TL1 | 7.03E−31 | 1.188 | 0.453 | 0.158 | 8.01E−27 |
| UBC | 7.27E−31 | 0.659 | 0.732 | 0.433 | 8.29E−27 |
| TNFRSF11B | 1.03E−30 | 0.639 | 0.146 | 0 | 1.17E−26 |
| ARHGAP32 | 1.03E−30 | 0.468 | 0.146 | 0 | 1.17E−26 |
| RNF152 | 1.08E−30 | 0.436 | 0.203 | 0.018 | 1.23E−26 |
| RPS7 | 2.01E−30 | 0.683 | 0.724 | 0.428 | 2.29E−26 |
| SLITRK6 | 2.32E−30 | 1.054 | 0.144 | 0 | 2.64E−26 |
| STEAP1 | 2.32E−30 | 0.494 | 0.144 | 0 | 2.64E−26 |
| ARHGAP29 | 5.46E−30 | 0.652 | 0.152 | 0.001 | 6.22E−26 |
| SERPINB6 | 5.83E−30 | 0.333 | 0.209 | 0.031 | 6.65E−26 |
| SPTY2D1-AS1 | 1.00E−29 | 0.338 | 0.152 | 0.001 | 1.15E−25 |
| MT1G | 1.18E−29 | 0.444 | 0.141 | 0 | 1.34E−25 |
| XRCC6BP1 | 1.34E−29 | 1.013 | 0.171 | 0.007 | 1.52E−25 |
| RPL3 | 2.12E−29 | 0.676 | 0.759 | 0.506 | 2.41E−25 |
| JMJD1C | 3.73E−29 | 0.951 | 0.515 | 0.221 | 4.25E−25 |
| SERGEF | 3.82E−29 | 0.428 | 0.182 | 0.013 | 4.36E−25 |
| CHCHD1 | 5.72E−29 | 0.832 | 0.422 | 0.155 | 6.52E−25 |
| DSG2 | 5.93E−29 | 0.554 | 0.138 | 0 | 6.76E−25 |
| SAMD8 | 5.97E−29 | 0.592 | 0.257 | 0.041 | 6.81E−25 |
| RPL10A | 8.19E−29 | 0.623 | 0.81 | 0.539 | 9.33E−25 |
| AP3M1 | 8.39E−29 | 0.472 | 0.257 | 0.045 | 9.56E−25 |
| C1orf64 | 1.00E−28 | 0.402 | 0.147 | 0.001 | 1.14E−24 |
| PHEX | 1.33E−28 | 0.549 | 0.136 | 0 | 1.52E−24 |
| RPL39 | 2.51E−28 | 0.335 | 0.952 | 0.778 | 2.86E−24 |
| MB | 2.98E−28 | 0.475 | 0.135 | 0 | 3.40E−24 |
| AC104667.3 | 2.98E−28 | 0.465 | 0.135 | 0 | 3.40E−24 |
| LONP2 | 3.03E−28 | 0.331 | 0.304 | 0.083 | 3.46E−24 |
| SPTY2D1 | 3.21E−28 | 0.641 | 0.376 | 0.114 | 3.65E−24 |
| SEC24C | 3.23E−28 | 0.570 | 0.25 | 0.041 | 3.69E−24 |
| TMEM86A | 3.43E−28 | 0.358 | 0.135 | 0.003 | 3.91E−24 |
| CXXC5 | 3.92E−28 | 0.379 | 0.171 | 0.01 | 4.47E−24 |
| ITPR1 | 5.15E−28 | 0.508 | 0.19 | 0.018 | 5.87E−24 |
| F12 | 6.67E−28 | 0.491 | 0.133 | 0 | 7.60E−24 |
| PI15 | 6.67E−28 | 1.473 | 0.133 | 0 | 7.60E−24 |
| RSF1 | 7.67E−28 | 0.833 | 0.537 | 0.251 | 8.74E−24 |
| PVT1 | 8.49E−28 | 1.439 | 0.219 | 0.045 | 9.68E−24 |
| USP6NL | 9.23E−28 | 0.376 | 0.249 | 0.051 | 1.05E−23 |
| CTSF | 1.09E−27 | 0.448 | 0.19 | 0.016 | 1.25E−23 |
| WWP1 | 1.24E−27 | 0.763 | 0.35 | 0.099 | 1.41E−23 |
| SH3BP4 | 1.25E−27 | 0.330 | 0.19 | 0.025 | 1.43E−23 |
| ZBTB43 | 1.49E−27 | 1.180 | 0.271 | 0.061 | 1.70E−23 |
| RP13-895J2.7 | 1.49E−27 | 0.467 | 0.132 | 0 | 1.70E−23 |
| AFMID | 1.60E−27 | 0.571 | 0.227 | 0.032 | 1.82E−23 |
| GOLGA2 | 2.18E−27 | 0.565 | 0.369 | 0.118 | 2.49E−23 |
| SIGLEC15 | 3.33E−27 | 0.394 | 0.13 | 0 | 3.80E−23 |
| FAM83H | 3.33E−27 | 0.363 | 0.13 | 0 | 3.80E−23 |
| CYP4B1 | 3.33E−27 | 0.345 | 0.13 | 0 | 3.80E−23 |
| GALNT6 | 3.50E−27 | 0.469 | 0.168 | 0.01 | 3.99E−23 |
| RAC3 | 3.85E−27 | 0.295 | 0.147 | 0.007 | 4.39E−23 |
| OSBPL1A | 5.04E−27 | 0.305 | 0.219 | 0.047 | 5.75E−23 |
| DEGS2 | 5.17E−27 | 0.397 | 0.139 | 0.001 | 5.89E−23 |
| SRRM2 | 5.22E−27 | 0.634 | 0.471 | 0.186 | 5.95E−23 |
| TPT1 | 5.26E−27 | 0.565 | 0.946 | 0.782 | 5.99E−23 |
| RPL27A | 5.44E−27 | 0.443 | 0.945 | 0.798 | 6.20E−23 |
| S100A16 | 5.78E−27 | 0.266 | 0.323 | 0.113 | 6.59E−23 |
| RPL41 | 7.09E−27 | 0.493 | 0.924 | 0.768 | 8.08E−23 |
| PRSS8 | 7.91E−27 | 0.533 | 0.138 | 0.001 | 9.02E−23 |
| ECHDC3 | 9.91E−27 | 0.347 | 0.166 | 0.013 | 1.13E−22 |
| TSPAN14 | 1.01E−26 | 0.294 | 0.242 | 0.054 | 1.15E−22 |
| STXBP1 | 1.18E−26 | 0.918 | 0.151 | 0.006 | 1.34E−22 |
| GABRQ | 1.66E−26 | 0.571 | 0.127 | 0 | 1.89E−22 |
| TRIB3 | 1.68E−26 | 0.703 | 0.235 | 0.038 | 1.92E−22 |
| DLG5 | 3.54E−26 | 0.367 | 0.176 | 0.016 | 4.03E−22 |
| FAM129B | 4.72E−26 | 1.215 | 0.336 | 0.114 | 5.37E−22 |
| TFF3 | 5.85E−26 | 0.643 | 0.146 | 0.004 | 6.66E−22 |
| SLPI | 8.19E−26 | 0.559 | 0.124 | 0 | 9.34E−22 |
| ANKRD50 | 8.19E−26 | 0.415 | 0.124 | 0 | 9.34E−22 |
| RPS18 | 1.06E−25 | 0.642 | 0.973 | 0.912 | 1.21E−21 |
| C4orf3 | 1.08E−25 | 0.640 | 0.434 | 0.168 | 1.23E−21 |
| PPP3CA | 1.50E−25 | 0.746 | 0.279 | 0.061 | 1.71E−21 |
| MMP24-AS1 | 1.52E−25 | 0.450 | 0.214 | 0.031 | 1.73E−21 |
| PCBD1 | 1.64E−25 | 0.587 | 0.309 | 0.082 | 1.87E−21 |
| CSAG1 | 1.82E−25 | 0.299 | 0.122 | 0 | 2.07E−21 |
| OXSM | 2.00E−25 | 0.387 | 0.193 | 0.025 | 2.28E−21 |
| ZMYND8 | 3.39E−25 | 0.475 | 0.293 | 0.077 | 3.86E−21 |
| REEP6 | 4.04E−25 | 0.337 | 0.12 | 0 | 4.60E−21 |
| COMMD3 | 7.95E−25 | 0.610 | 0.25 | 0.048 | 9.06E−21 |
| TCAP | 8.94E−25 | 0.320 | 0.119 | 0 | 1.02E−20 |
| LRIG1 | 8.99E−25 | 0.480 | 0.193 | 0.025 | 1.03E−20 |
| PITRM1 | 1.11E−24 | 0.572 | 0.311 | 0.088 | 1.27E−20 |
| PDXDC1 | 1.25E−24 | 0.340 | 0.376 | 0.142 | 1.42E−20 |
| INHBB | 1.35E−24 | 0.306 | 0.138 | 0.006 | 1.54E−20 |
| SFN | 1.35E−24 | 0.421 | 0.127 | 0.001 | 1.54E−20 |
| LMO4 | 1.41E−24 | 0.448 | 0.542 | 0.262 | 1.61E−20 |
| EPAS1 | 1.57E−24 | 0.344 | 0.144 | 0.009 | 1.78E−20 |
| GSTT1 | 1.98E−24 | 0.281 | 0.117 | 0 | 2.26E−20 |
| NME3 | 2.30E−24 | 0.283 | 0.201 | 0.041 | 2.62E−20 |
| SYNGR2 | 2.35E−24 | 0.793 | 0.547 | 0.333 | 2.67E−20 |
| UEVLD | 2.58E−24 | 0.368 | 0.212 | 0.037 | 2.94E−20 |
| ECI2 | 3.20E−24 | 0.426 | 0.225 | 0.041 | 3.64E−20 |
| TRIM44 | 3.25E−24 | 1.277 | 0.317 | 0.108 | 3.71E−20 |
| IL8 | 4.11E−24 | 0.986 | 0.176 | 0.018 | 4.68E−20 |
| EEF1A1 | 4.31E−24 | 0.483 | 0.861 | 0.649 | 4.91E−20 |
| STC2 | 4.38E−24 | 0.445 | 0.116 | 0 | 4.99E−20 |
| TGFB3 | 4.38E−24 | 0.344 | 0.116 | 0 | 4.99E−20 |
| CHMP3 | 5.35E−24 | 0.398 | 0.403 | 0.159 | 6.10E−20 |
| DLGAP1-AS1 | 7.58E−24 | 0.412 | 0.17 | 0.016 | 8.64E−20 |
| LRSAM1 | 8.59E−24 | 0.837 | 0.166 | 0.018 | 9.79E−20 |
| NDUFC2 | 9.32E−24 | 0.718 | 0.521 | 0.344 | 1.06E−19 |
| VAPA | 1.01E−23 | 0.566 | 0.487 | 0.218 | 1.15E−19 |
| TSPAN9 | 1.21E−23 | 0.305 | 0.135 | 0.007 | 1.38E−19 |
| SPIRE1 | 1.22E−23 | 0.449 | 0.195 | 0.031 | 1.39E−19 |
| CRAT | 1.54E−23 | 0.362 | 0.163 | 0.015 | 1.76E−19 |
| RBM39 | 1.60E−23 | 0.658 | 0.583 | 0.307 | 1.83E−19 |
| TIMP3 | 1.94E−23 | 0.926 | 0.122 | 0.001 | 2.21E−19 |
| UNC5B | 2.13E−23 | 0.356 | 0.113 | 0 | 2.43E−19 |
| PPAPDC1B | 2.83E−23 | 0.456 | 0.238 | 0.05 | 3.23E−19 |
| CD36 | 3.28E−23 | 0.551 | 0.127 | 0.003 | 3.73E−19 |
| TRIB1 | 3.38E−23 | 0.751 | 0.379 | 0.137 | 3.85E−19 |
| TSG101 | 3.58E−23 | 0.326 | 0.35 | 0.133 | 4.09E−19 |
| ORAOV1 | 4.33E−23 | 0.401 | 0.166 | 0.016 | 4.93E−19 |
| FGFR4 | 4.71E−23 | 0.263 | 0.111 | 0 | 5.36E−19 |
| RANBP9 | 6.27E−23 | 0.390 | 0.319 | 0.102 | 7.15E−19 |
| ASPH | 6.39E−23 | 0.555 | 0.468 | 0.209 | 7.29E−19 |
| SYT12 | 8.20E−23 | 0.317 | 0.12 | 0.001 | 9.35E−19 |
| FGD5-AS1 | 9.01E−23 | 0.577 | 0.396 | 0.158 | 1.03E−18 |
| RPL30 | 9.80E−23 | 0.300 | 0.989 | 0.947 | 1.12E−18 |
| FADD | 1.20E−22 | 0.291 | 0.204 | 0.042 | 1.37E−18 |
| IGFBP5 | 1.62E−22 | 0.823 | 0.152 | 0.013 | 1.85E−18 |
| FGB | 2.28E−22 | 1.272 | 0.108 | 0 | 2.60E−18 |
| NOVA1 | 2.28E−22 | 0.812 | 0.108 | 0 | 2.60E−18 |
| SGPL1 | 2.39E−22 | 0.334 | 0.144 | 0.013 | 2.72E−18 |
| MYLIP | 2.84E−22 | 0.420 | 0.174 | 0.022 | 3.23E−18 |
| CRNKL1 | 3.08E−22 | 0.669 | 0.282 | 0.075 | 3.51E−18 |
| ZG16B | 4.06E−22 | 0.567 | 0.116 | 0.001 | 4.63E−18 |
| TOB2 | 4.14E−22 | 0.483 | 0.284 | 0.079 | 4.72E−18 |
| CRABP1 | 5.01E−22 | 0.701 | 0.106 | 0 | 5.71E−18 |
| ERMN | 5.01E−22 | 0.361 | 0.106 | 0 | 5.71E−18 |
| BTG2 | 5.04E−22 | 0.685 | 0.246 | 0.056 | 5.75E−18 |
| DDIT4 | 5.51E−22 | 0.571 | 0.314 | 0.098 | 6.28E−18 |
| RAD50 | 6.17E−22 | 0.519 | 0.349 | 0.123 | 7.03E−18 |
| FTH1 | 7.54E−22 | 0.490 | 0.906 | 0.711 | 8.59E−18 |
| XPC | 8.80E−22 | 0.384 | 0.249 | 0.064 | 1.00E−17 |
| METTL1 | 9.14E−22 | 0.995 | 0.193 | 0.035 | 1.04E−17 |
| SLC22A23 | 9.82E−22 | 0.306 | 0.124 | 0.007 | 1.12E−17 |
| KDM2A | 1.10E−21 | 0.543 | 0.312 | 0.096 | 1.26E−17 |
| VHL | 1.37E−21 | 0.523 | 0.295 | 0.091 | 1.56E−17 |
| SQSTM1 | 1.70E−21 | 0.379 | 0.523 | 0.268 | 1.94E−17 |
| PLEKHF2 | 1.94E−21 | 0.720 | 0.339 | 0.115 | 2.21E−17 |
| TMEM141 | 2.06E−21 | 0.525 | 0.36 | 0.13 | 2.35E−17 |
| TARS | 2.59E−21 | 0.535 | 0.453 | 0.202 | 2.95E−17 |
| PPP1R15A | 2.60E−21 | 0.593 | 0.355 | 0.126 | 2.97E−17 |
| CLU | 3.63E−21 | 0.403 | 0.379 | 0.152 | 4.14E−17 |
| NOL7 | 4.45E−21 | 0.644 | 0.599 | 0.348 | 5.07E−17 |
| CP | 4.79E−21 | 1.478 | 0.325 | 0.197 | 5.46E−17 |
| LARP7 | 4.87E−21 | 0.523 | 0.344 | 0.123 | 5.55E−17 |
| RHOB | 5.31E−21 | 0.441 | 0.401 | 0.167 | 6.05E−17 |
| MT-TY | 5.65E−21 | 0.362 | 0.109 | 0.001 | 6.44E−17 |
| CAPN7 | 7.72E−21 | 0.389 | 0.277 | 0.086 | 8.80E−17 |
| CLNS1A | 7.77E−21 | 0.568 | 0.403 | 0.171 | 8.85E−17 |
| CIRBP | 8.10E−21 | 0.482 | 0.374 | 0.143 | 9.23E−17 |
| IL13RA1 | 8.59E−21 | 0.288 | 0.241 | 0.072 | 9.79E−17 |
| MAN1A2 | 1.25E−20 | 0.480 | 0.429 | 0.19 | 1.42E−16 |
| EEF1B2 | 1.28E−20 | 0.466 | 0.726 | 0.465 | 1.46E−16 |
| G6PD | 1.46E−20 | 0.297 | 0.197 | 0.042 | 1.67E−16 |
| PON2 | 1.55E−20 | 0.400 | 0.139 | 0.016 | 1.77E−16 |
| CLCN3 | 1.57E−20 | 0.350 | 0.276 | 0.086 | 1.79E−16 |
| ZFP36 | 1.76E−20 | 0.351 | 0.428 | 0.193 | 2.00E−16 |
| HILPDA | 1.97E−20 | 0.628 | 0.257 | 0.066 | 2.24E−16 |
| IRX5 | 2.07E−20 | 0.563 | 0.143 | 0.012 | 2.36E−16 |
| BLVRB | 2.50E−20 | 0.367 | 0.233 | 0.06 | 2.85E−16 |
| RABEP1 | 2.88E−20 | 0.795 | 0.371 | 0.145 | 3.29E−16 |
| CADPS2 | 3.46E−20 | 0.349 | 0.122 | 0.009 | 3.94E−16 |
| RPL13 | 3.69E−20 | 0.264 | 0.984 | 0.895 | 4.21E−16 |
| COX7A2L | 3.69E−20 | 0.321 | 0.418 | 0.192 | 4.21E−16 |
| SEPHS2 | 4.62E−20 | 0.353 | 0.315 | 0.114 | 5.27E−16 |
| ADK | 5.32E−20 | 0.559 | 0.366 | 0.14 | 6.06E−16 |
| PRSS23 | 5.65E−20 | 0.567 | 0.208 | 0.041 | 6.44E−16 |
| ARHGEF38 | 6.45E−20 | 0.367 | 0.106 | 0.001 | 7.35E−16 |
| CX3CL1 | 7.49E−20 | 0.674 | 0.105 | 0.001 | 8.54E−16 |
| FAH | 7.54E−20 | 0.305 | 0.165 | 0.025 | 8.59E−16 |
| SPTSSA | 8.24E−20 | 0.326 | 0.358 | 0.148 | 9.40E−16 |
| RARRES3 | 8.35E−20 | 0.470 | 0.149 | 0.019 | 9.52E−16 |
| ZMIZ1 | 8.95E−20 | 0.561 | 0.3 | 0.095 | 1.02E−15 |
| TMEM159 | 9.24E−20 | 0.272 | 0.219 | 0.058 | 1.05E−15 |
| RPS8 | 1.06E−19 | 0.299 | 0.987 | 0.95 | 1.20E−15 |
| ST6GALNAC4 | 1.17E−19 | 0.794 | 0.113 | 0.004 | 1.34E−15 |
| ATP8B1 | 1.25E−19 | 0.697 | 0.132 | 0.009 | 1.42E−15 |
| JUN | 1.32E−19 | 0.651 | 0.609 | 0.357 | 1.51E−15 |
| ATF4 | 1.60E−19 | 0.528 | 0.534 | 0.284 | 1.82E−15 |
| SCAND1 | 1.75E−19 | 0.266 | 0.352 | 0.148 | 1.99E−15 |
| EIF4B | 2.47E−19 | 0.400 | 0.41 | 0.186 | 2.82E−15 |
| RPL29 | 2.58E−19 | 0.548 | 0.571 | 0.314 | 2.94E−15 |
| RPL18A | 2.85E−19 | 0.647 | 0.464 | 0.238 | 3.24E−15 |
| EGR2 | 3.39E−19 | 0.566 | 0.174 | 0.026 | 3.87E−15 |
| GSE1 | 3.85E−19 | 0.495 | 0.209 | 0.044 | 4.39E−15 |
| RAB25 | 4.04E−19 | 0.307 | 0.101 | 0.001 | 4.61E−15 |
| SPRY1 | 4.31E−19 | 0.363 | 0.132 | 0.012 | 4.92E−15 |
| GARNL3 | 4.51E−19 | 0.731 | 0.101 | 0.001 | 5.14E−15 |
| SCD | 4.93E−19 | 0.764 | 0.599 | 0.37 | 5.61E−15 |
| PTP4A2 | 5.00E−19 | 0.545 | 0.431 | 0.194 | 5.70E−15 |
| GADD45B | 5.50E−19 | 0.729 | 0.38 | 0.159 | 6.27E−15 |
| FEZ2 | 5.55E−19 | 0.422 | 0.22 | 0.053 | 6.33E−15 |
| PSMD3 | 7.39E−19 | 0.401 | 0.322 | 0.123 | 8.42E−15 |
| HID1 | 7.60E−19 | 0.324 | 0.117 | 0.006 | 8.67E−15 |
| EDF1 | 7.62E−19 | 0.358 | 0.493 | 0.257 | 8.69E−15 |
| ARFIP2 | 7.93E−19 | 0.347 | 0.17 | 0.026 | 9.04E−15 |
| TMEM66 | 9.59E−19 | 0.392 | 0.458 | 0.225 | 1.09E−14 |
| EFHD1 | 9.80E−19 | 0.391 | 0.258 | 0.076 | 1.12E−14 |
| RBCK1 | 1.02E−18 | 0.518 | 0.211 | 0.047 | 1.16E−14 |
| CDK4 | 1.11E−18 | 0.995 | 0.352 | 0.164 | 1.27E−14 |
| ATP2C2 | 1.19E−18 | 0.284 | 0.152 | 0.023 | 1.35E−14 |
| GPX4 | 1.78E−18 | 0.312 | 0.613 | 0.367 | 2.03E−14 |
| TMEM144 | 1.78E−18 | 0.396 | 0.109 | 0.004 | 2.03E−14 |
| XRRA1 | 1.93E−18 | 0.358 | 0.163 | 0.025 | 2.20E−14 |
| GUCY1A2 | 2.67E−18 | 0.402 | 0.147 | 0.018 | 3.04E−14 |
| SIRT5 | 2.78E−18 | 0.416 | 0.209 | 0.048 | 3.16E−14 |
| CMBL | 2.98E−18 | 0.266 | 0.163 | 0.029 | 3.40E−14 |
| ZNF148 | 3.29E−18 | 0.359 | 0.216 | 0.063 | 3.75E−14 |
| PARD6B | 4.04E−18 | 0.262 | 0.135 | 0.019 | 4.60E−14 |
| ASS1 | 5.09E−18 | 0.406 | 0.326 | 0.126 | 5.80E−14 |
| NRBF2 | 5.76E−18 | 0.275 | 0.239 | 0.076 | 6.56E−14 |
| MTHFD2 | 6.73E−18 | 0.516 | 0.433 | 0.202 | 7.67E−14 |
| MS4A7 | 8.08E−18 | 0.451 | 0.116 | 0.007 | 9.21E−14 |
| BCAM | 8.62E−18 | 0.489 | 0.101 | 0.003 | 9.82E−14 |
| EMP2 | 8.71E−18 | 0.415 | 0.311 | 0.117 | 9.93E−14 |
| ARID5B | 1.09E−17 | 0.335 | 0.268 | 0.091 | 1.24E−13 |
| PHKB | 1.10E−17 | 0.306 | 0.225 | 0.066 | 1.25E−13 |
| EIF3M | 1.13E−17 | 0.293 | 0.439 | 0.227 | 1.29E−13 |
| DDX17 | 1.51E−17 | 0.314 | 0.575 | 0.336 | 1.72E−13 |
| CAMK2G | 1.57E−17 | 0.311 | 0.182 | 0.041 | 1.79E−13 |
| ETFA | 1.67E−17 | 0.250 | 0.261 | 0.095 | 1.90E−13 |
| TBC1D9 | 1.76E−17 | 0.678 | 0.171 | 0.029 | 2.00E−13 |
| TSC22D3 | 2.20E−17 | 0.484 | 0.166 | 0.031 | 2.51E−13 |
| MYO10 | 2.91E−17 | 0.287 | 0.147 | 0.026 | 3.32E−13 |
| NGLY1 | 3.23E−17 | 0.400 | 0.228 | 0.061 | 3.68E−13 |
| FAM198B | 3.35E−17 | 0.725 | 0.113 | 0.007 | 3.82E−13 |
| CPT1A | 4.66E−17 | 0.386 | 0.223 | 0.064 | 5.31E−13 |
| ATP6V1G1 | 5.54E−17 | 0.533 | 0.539 | 0.301 | 6.31E−13 |
| NIPBL | 7.20E−17 | 0.466 | 0.423 | 0.202 | 8.21E−13 |
| ZFAND6 | 8.01E−17 | 0.332 | 0.219 | 0.064 | 9.13E−13 |
| CTSD | 8.36E−17 | 0.334 | 0.344 | 0.155 | 9.52E−13 |
| TMED3 | 8.86E−17 | 0.414 | 0.295 | 0.108 | 1.01E−12 |
| DHTKD1 | 1.24E−16 | 0.265 | 0.228 | 0.075 | 1.42E−12 |
| SRSF5 | 1.28E−16 | 0.273 | 0.388 | 0.184 | 1.46E−12 |
| VTRNA1-1 | 1.49E−16 | 0.534 | 0.189 | 0.041 | 1.70E−12 |
| NDRG1 | 1.88E−16 | 0.847 | 0.303 | 0.113 | 2.15E−12 |
| ME1 | 2.02E−16 | 0.577 | 0.127 | 0.015 | 2.30E−12 |
| FOXP1 | 2.41E−16 | 0.449 | 0.225 | 0.063 | 2.75E−12 |
| UBE2Q2 | 2.70E−16 | 0.448 | 0.212 | 0.058 | 3.08E−12 |
| SNHG9 | 2.74E−16 | 0.269 | 0.285 | 0.113 | 3.12E−12 |
| TTC28-AS1 | 3.58E−16 | 0.360 | 0.128 | 0.016 | 4.08E−12 |
| RIN2 | 3.93E−16 | 0.370 | 0.117 | 0.012 | 4.48E−12 |
| RBM47 | 4.78E−16 | 0.470 | 0.277 | 0.098 | 5.45E−12 |
| PAPSS2 | 5.02E−16 | 0.515 | 0.125 | 0.013 | 5.72E−12 |
| PFDN6 | 5.06E−16 | 0.358 | 0.345 | 0.152 | 5.76E−12 |
| STARD10 | 5.27E−16 | 0.418 | 0.235 | 0.07 | 6.01E−12 |
| ST13 | 5.39E−16 | 0.339 | 0.509 | 0.288 | 6.14E−12 |
| PEBP1 | 5.80E−16 | 0.336 | 0.597 | 0.418 | 6.61E−12 |
| MAGI3 | 6.55E−16 | 0.272 | 0.106 | 0.012 | 7.47E−12 |
| GADD45A | 6.81E−16 | 0.709 | 0.165 | 0.031 | 7.76E−12 |
| SEC13 | 7.24E−16 | 0.286 | 0.304 | 0.127 | 8.25E−12 |
| GSPT1 | 7.57E−16 | 0.276 | 0.398 | 0.197 | 8.63E−12 |
| SOCS3 | 8.99E−16 | 0.254 | 0.119 | 0.013 | 1.02E−11 |
| HERPUD1 | 9.21E−16 | 0.346 | 0.287 | 0.11 | 1.05E−11 |
| PDHX | 1.07E−15 | 1.053 | 0.271 | 0.152 | 1.22E−11 |
| EIF3L | 1.10E−15 | 0.413 | 0.49 | 0.269 | 1.26E−11 |
| TTC3 | 1.42E−15 | 0.434 | 0.572 | 0.349 | 1.61E−11 |
| TMEM241 | 1.42E−15 | 0.409 | 0.16 | 0.029 | 1.62E−11 |
| CTSH | 1.44E−15 | 0.383 | 0.177 | 0.047 | 1.64E−11 |
| ZNF350 | 1.48E−15 | 0.293 | 0.136 | 0.02 | 1.68E−11 |
| RABL5 | 2.20E−15 | 0.335 | 0.13 | 0.018 | 2.50E−11 |
| RPS27L | 2.24E−15 | 0.257 | 0.42 | 0.221 | 2.55E−11 |
| DYNC2LI1 | 2.65E−15 | 0.301 | 0.106 | 0.012 | 3.02E−11 |
| FASN | 3.04E−15 | 0.470 | 0.357 | 0.159 | 3.46E−11 |
| CSDE1 | 3.30E−15 | 0.294 | 0.634 | 0.408 | 3.76E−11 |
| ABCA5 | 3.40E−15 | 0.416 | 0.185 | 0.044 | 3.88E−11 |
| WDR70 | 3.58E−15 | 0.581 | 0.241 | 0.076 | 4.08E−11 |
| PTRHD1 | 4.44E−15 | 0.270 | 0.254 | 0.094 | 5.07E−11 |
| RNF187 | 5.08E−15 | 0.274 | 0.19 | 0.053 | 5.79E−11 |
| C17orf89 | 5.57E−15 | 0.357 | 0.355 | 0.165 | 6.35E−11 |
| OAZ2 | 5.94E−15 | 0.355 | 0.201 | 0.056 | 6.77E−11 |
| USP40 | 6.37E−15 | 0.361 | 0.139 | 0.022 | 7.27E−11 |
| TTC39A | 6.42E−15 | 0.450 | 0.152 | 0.028 | 7.32E−11 |
| MT-TP | 6.73E−15 | 0.458 | 0.469 | 0.251 | 7.67E−11 |
| SNHG7 | 6.89E−15 | 0.317 | 0.231 | 0.077 | 7.85E−11 |
| DSTN | 6.95E−15 | 0.409 | 0.758 | 0.569 | 7.92E−11 |
| LINC00493 | 7.68E−15 | 0.278 | 0.366 | 0.175 | 8.75E−11 |
| SRGN | 7.81E−15 | 1.074 | 0.26 | 0.105 | 8.90E−11 |
| INADL | 8.04E−15 | 0.267 | 0.263 | 0.104 | 9.17E−11 |
| MCUR1 | 8.27E−15 | 0.428 | 0.35 | 0.158 | 9.43E−11 |
| HN1L | 8.34E−15 | 0.264 | 0.319 | 0.146 | 9.50E−11 |
| RTN4 | 8.97E−15 | 0.252 | 0.472 | 0.268 | 1.02E−10 |
| MXI1 | 1.05E−14 | 0.403 | 0.166 | 0.037 | 1.20E−10 |
| ST6GAL1 | 1.15E−14 | 0.351 | 0.2 | 0.054 | 1.31E−10 |
| MKRN1 | 1.41E−14 | 0.279 | 0.197 | 0.057 | 1.61E−10 |
| GLTSCR2 | 1.53E−14 | 0.333 | 0.472 | 0.262 | 1.74E−10 |
| TTC14 | 1.61E−14 | 0.404 | 0.176 | 0.042 | 1.83E−10 |
| REEP3 | 1.68E−14 | 0.373 | 0.341 | 0.155 | 1.92E−10 |
| DCXR | 1.85E−14 | 0.267 | 0.339 | 0.162 | 2.11E−10 |
| DNAJC1 | 1.96E−14 | 0.424 | 0.304 | 0.126 | 2.24E−10 |
| ASNS | 2.04E−14 | 0.396 | 0.192 | 0.051 | 2.32E−10 |
| EGLN3 | 2.05E−14 | 0.518 | 0.158 | 0.032 | 2.33E−10 |
| ANKRD12 | 2.13E−14 | 0.411 | 0.436 | 0.228 | 2.43E−10 |
| TRIB2 | 2.20E−14 | 0.311 | 0.255 | 0.096 | 2.51E−10 |
| PRRG4 | 2.58E−14 | 0.321 | 0.144 | 0.029 | 2.94E−10 |
| SAPCD2 | 3.00E−14 | 0.274 | 0.174 | 0.047 | 3.42E−10 |
| FUT11 | 3.16E−14 | 0.417 | 0.143 | 0.025 | 3.60E−10 |
| MBOAT2 | 3.36E−14 | 0.318 | 0.203 | 0.06 | 3.83E−10 |
| BAIAP2 | 3.61E−14 | 0.277 | 0.144 | 0.028 | 4.12E−10 |
| RPL8 | 4.03E−14 | 0.347 | 0.924 | 0.825 | 4.59E−10 |
| REEP5 | 4.09E−14 | 0.389 | 0.357 | 0.17 | 4.66E−10 |
| CTA-29F11.1 | 5.32E−14 | 0.258 | 0.12 | 0.018 | 6.06E−10 |
| RSL1D1 | 5.74E−14 | 0.261 | 0.58 | 0.367 | 6.54E−10 |
| PPP1R35 | 6.58E−14 | 0.253 | 0.135 | 0.023 | 7.50E−10 |
| TSPAN31 | 6.60E−14 | 0.436 | 0.149 | 0.029 | 7.52E−10 |
| PDE4D | 6.77E−14 | 0.301 | 0.133 | 0.025 | 7.72E−10 |
| PPDPF | 6.78E−14 | 0.470 | 0.567 | 0.406 | 7.73E−10 |
| RBM3 | 7.43E−14 | 0.530 | 0.475 | 0.294 | 8.47E−10 |
| NACA | 7.76E−14 | 0.393 | 0.697 | 0.484 | 8.85E−10 |
| BZW1 | 8.24E−14 | 0.442 | 0.472 | 0.262 | 9.39E−10 |
| SREBF1 | 9.43E−14 | 0.418 | 0.19 | 0.051 | 1.07E−09 |
| GNB2L1 | 9.45E−14 | 0.389 | 0.832 | 0.667 | 1.08E−09 |
| RPS29 | 1.06E−13 | 0.534 | 0.973 | 0.934 | 1.21E−09 |
| LINC00667 | 1.07E−13 | 0.279 | 0.119 | 0.016 | 1.22E−09 |
| MSL1 | 1.16E−13 | 0.334 | 0.336 | 0.158 | 1.32E−09 |
| RPL4 | 1.26E−13 | 0.338 | 0.813 | 0.64 | 1.43E−09 |
| ASRGL1 | 1.26E−13 | 0.276 | 0.12 | 0.018 | 1.44E−09 |
| CTDSP2 | 1.26E−13 | 0.651 | 0.19 | 0.057 | 1.44E−09 |
| APMAP | 1.48E−13 | 0.442 | 0.249 | 0.089 | 1.68E−09 |
| DDAH1 | 1.64E−13 | 0.439 | 0.133 | 0.026 | 1.87E−09 |
| EPB41L4A | 1.83E−13 | 0.291 | 0.143 | 0.029 | 2.09E−09 |
| SYTL2 | 1.86E−13 | 0.433 | 0.117 | 0.016 | 2.12E−09 |
| HLTF | 1.86E−13 | 0.352 | 0.246 | 0.092 | 2.12E−09 |
| N4BP2L2 | 1.90E−13 | 0.356 | 0.374 | 0.189 | 2.17E−09 |
| UACA | 1.94E−13 | 0.439 | 0.124 | 0.022 | 2.22E−09 |
| CCDC174 | 1.97E−13 | 0.303 | 0.212 | 0.072 | 2.24E−09 |
| LEPROT | 2.53E−13 | 0.251 | 0.166 | 0.044 | 2.89E−09 |
| CPNE3 | 2.72E−13 | 0.405 | 0.464 | 0.26 | 3.10E−09 |
| RALBP1 | 2.76E−13 | 0.261 | 0.342 | 0.171 | 3.14E−09 |
| EIF4EBP1 | 3.07E−13 | 0.337 | 0.357 | 0.175 | 3.49E−09 |
| FPGS | 3.14E−13 | 0.771 | 0.162 | 0.045 | 3.58E−09 |
| CLK1 | 3.35E−13 | 0.450 | 0.273 | 0.107 | 3.82E−09 |
| PBX1 | 3.55E−13 | 0.563 | 0.379 | 0.196 | 4.05E−09 |
| ANKRD36C | 4.33E−13 | 1.243 | 0.298 | 0.184 | 4.93E−09 |
| RMDN1 | 5.13E−13 | 0.370 | 0.254 | 0.096 | 5.84E−09 |
| TNRC6C | 5.61E−13 | 0.323 | 0.155 | 0.037 | 6.39E−09 |
| PEX11A | 5.82E−13 | 0.315 | 0.113 | 0.016 | 6.63E−09 |
| SLC2A8 | 6.09E−13 | 0.607 | 0.127 | 0.023 | 6.94E−09 |
| CAST | 6.50E−13 | 0.395 | 0.461 | 0.259 | 7.41E−09 |
| RBM27 | 7.01E−13 | 0.347 | 0.3 | 0.132 | 7.99E−09 |
| BHLHE40 | 8.96E−13 | 0.454 | 0.206 | 0.067 | 1.02E−08 |
| TRAF3IP1 | 9.83E−13 | 0.282 | 0.146 | 0.035 | 1.12E−08 |
| ALDH2 | 1.06E−12 | 0.355 | 0.184 | 0.054 | 1.21E−08 |
| DDT | 1.12E−12 | 0.403 | 0.355 | 0.173 | 1.28E−08 |
| EIF2S2 | 1.40E−12 | 0.298 | 0.553 | 0.361 | 1.60E−08 |
| GCC2 | 1.49E−12 | 0.358 | 0.326 | 0.155 | 1.69E−08 |
| CPEB4 | 1.57E−12 | 0.438 | 0.211 | 0.069 | 1.79E−08 |
| ARFGEF2 | 1.87E−12 | 0.378 | 0.242 | 0.092 | 2.13E−08 |
| ACAA1 | 2.13E−12 | 0.307 | 0.154 | 0.039 | 2.43E−08 |
| MRPL41 | 2.14E−12 | 0.262 | 0.309 | 0.151 | 2.44E−08 |
| BLCAP | 2.19E−12 | 0.347 | 0.152 | 0.035 | 2.50E−08 |
| VPS26A | 2.56E−12 | 0.473 | 0.415 | 0.225 | 2.92E−08 |
| BSPRY | 2.60E−12 | 0.376 | 0.143 | 0.031 | 2.96E−08 |
| SLC39A6 | 2.87E−12 | 0.573 | 0.214 | 0.079 | 3.27E−08 |
| PTRH1 | 2.87E−12 | 0.699 | 0.154 | 0.039 | 3.27E−08 |
| WDR83OS | 2.90E−12 | 0.410 | 0.453 | 0.257 | 3.30E−08 |
| HPS5 | 3.50E−12 | 0.290 | 0.168 | 0.05 | 3.99E−08 |
| DNAJB1 | 3.69E−12 | 0.608 | 0.493 | 0.31 | 4.20E−08 |
| PALMD | 3.86E−12 | 0.495 | 0.124 | 0.022 | 4.40E−08 |
| CRNDE | 4.20E−12 | 0.334 | 0.208 | 0.075 | 4.78E−08 |
| NFIC | 5.36E−12 | 0.365 | 0.236 | 0.091 | 6.10E−08 |
| DAAM1 | 5.37E−12 | 0.447 | 0.265 | 0.108 | 6.13E−08 |
| PTPN1 | 5.59E−12 | 0.306 | 0.247 | 0.101 | 6.37E−08 |
| COMT | 5.76E−12 | 0.293 | 0.304 | 0.143 | 6.57E−08 |
| MTUS1 | 6.27E−12 | 0.603 | 0.241 | 0.092 | 7.14E−08 |
| C6orf211 | 6.76E−12 | 0.579 | 0.125 | 0.023 | 7.70E−08 |
| MORF4L1 | 1.19E−11 | 0.260 | 0.42 | 0.243 | 1.35E−07 |
| PPIL4 | 1.19E−11 | 0.376 | 0.208 | 0.073 | 1.35E−07 |
| RBL2 | 1.38E−11 | 0.266 | 0.195 | 0.067 | 1.58E−07 |
| RPLP1 | 1.41E−11 | 0.321 | 0.976 | 0.943 | 1.61E−07 |
| SLC39A7 | 1.47E−11 | 0.274 | 0.322 | 0.162 | 1.67E−07 |
| FRG1 | 1.57E−11 | 0.299 | 0.345 | 0.175 | 1.79E−07 |
| SLTM | 1.64E−11 | 0.371 | 0.372 | 0.197 | 1.87E−07 |
| AK1 | 2.28E−11 | 0.482 | 0.103 | 0.015 | 2.60E−07 |
| SUB1 | 2.30E−11 | 0.448 | 0.612 | 0.462 | 2.62E−07 |
| SNHG3 | 2.40E−11 | 0.346 | 0.276 | 0.121 | 2.74E−07 |
| ZKSCAN1 | 4.27E−11 | 0.416 | 0.255 | 0.108 | 4.87E−07 |
| PPA2 | 4.75E−11 | 0.303 | 0.242 | 0.102 | 5.42E−07 |
| LARS | 5.34E−11 | 0.286 | 0.434 | 0.256 | 6.08E−07 |
| GLUD1 | 6.26E−11 | 0.313 | 0.25 | 0.108 | 7.14E−07 |
| LSM14B | 7.79E−11 | 0.318 | 0.154 | 0.042 | 8.87E−07 |
| RPL22 | 8.60E−11 | 0.424 | 0.594 | 0.406 | 9.80E−07 |
| RBM25 | 8.63E−11 | 0.269 | 0.506 | 0.326 | 9.84E−07 |
| KIAA1109 | 1.24E−10 | 0.302 | 0.209 | 0.08 | 1.41E−06 |
| DNAH5 | 1.36E−10 | 0.370 | 0.101 | 0.016 | 1.55E−06 |
| FAM84B | 1.41E−10 | 0.267 | 0.246 | 0.107 | 1.61E−06 |
| CHMP1B | 1.48E−10 | 0.406 | 0.292 | 0.136 | 1.68E−06 |
| IMPACT | 1.51E−10 | 0.277 | 0.165 | 0.054 | 1.72E−06 |
| HMGCS1 | 1.72E−10 | 0.434 | 0.376 | 0.23 | 1.96E−06 |
| HBP1 | 1.80E−10 | 0.341 | 0.146 | 0.039 | 2.05E−06 |
| DECR1 | 2.07E−10 | 0.253 | 0.185 | 0.07 | 2.36E−06 |
| PRDX2 | 2.14E−10 | 0.321 | 0.494 | 0.31 | 2.44E−06 |
| RASEF | 2.56E−10 | 0.283 | 0.103 | 0.019 | 2.92E−06 |
| HNRNPC | 3.26E−10 | 0.280 | 0.558 | 0.373 | 3.72E−06 |
| VEGFA | 4.26E−10 | 0.429 | 0.282 | 0.132 | 4.86E−06 |
| AKAP9 | 4.37E−10 | 0.319 | 0.493 | 0.354 | 4.98E−06 |
| GPCPD1 | 4.40E−10 | 0.535 | 0.139 | 0.037 | 5.01E−06 |
| PIM3 | 5.34E−10 | 0.314 | 0.2 | 0.076 | 6.09E−06 |
| EGR3 | 6.76E−10 | 0.697 | 0.108 | 0.026 | 7.70E−06 |
| WWC2 | 7.42E−10 | 0.294 | 0.12 | 0.028 | 8.46E−06 |
| NFKBIA | 9.06E−10 | 0.508 | 0.333 | 0.174 | 1.03E−05 |
| KTN1 | 1.26E−09 | 0.295 | 0.61 | 0.453 | 1.43E−05 |
| CHD6 | 1.27E−09 | 0.267 | 0.185 | 0.072 | 1.44E−05 |
| SMARCC1 | 1.49E−09 | 0.281 | 0.407 | 0.24 | 1.70E−05 |
| MYO6 | 3.07E−09 | 0.481 | 0.458 | 0.295 | 3.49E−05 |
| CSTB | 3.37E−09 | 0.376 | 0.471 | 0.385 | 3.84E−05 |
| ARFGAP3 | 3.47E−09 | 0.408 | 0.239 | 0.108 | 3.96E−05 |
| ALCAM | 3.57E−09 | 0.508 | 0.277 | 0.135 | 4.07E−05 |
| C9orf78 | 3.79E−09 | 0.333 | 0.347 | 0.197 | 4.32E−05 |
| SEMA3C | 3.87E−09 | 0.419 | 0.187 | 0.07 | 4.41E−05 |
| LSM3 | 4.65E−09 | 0.314 | 0.504 | 0.341 | 5.29E−05 |
| PEX13 | 6.13E−09 | 0.264 | 0.101 | 0.02 | 6.99E−05 |
| SLC25A25 | 6.78E−09 | 0.431 | 0.146 | 0.048 | 7.73E−05 |
| TNRC6B | 7.08E−09 | 0.301 | 0.268 | 0.136 | 8.07E−05 |
| STAT1 | 8.10E−09 | 0.298 | 0.304 | 0.164 | 9.23E−05 |
| GOLGA4 | 9.04E−09 | 0.302 | 0.542 | 0.37 | 1.03E−04 |
| NCOA3 | 1.00E−08 | 0.338 | 0.203 | 0.083 | 1.14E−04 |
| UIMC1 | 1.26E−08 | 0.254 | 0.152 | 0.053 | 1.44E−04 |
| H1FX | 1.43E−08 | 0.277 | 0.209 | 0.091 | 1.63E−04 |
| RPAP2 | 1.46E−08 | 0.326 | 0.174 | 0.066 | 1.67E−04 |
| BCLAF1 | 1.49E−08 | 0.271 | 0.387 | 0.232 | 1.70E−04 |
| RTKN2 | 1.58E−08 | 0.333 | 0.204 | 0.089 | 1.80E−04 |
| DRG1 | 1.70E−08 | 0.295 | 0.163 | 0.058 | 1.94E−04 |
| SDC1 | 1.78E−08 | 0.343 | 0.197 | 0.08 | 2.03E−04 |
| RN7SL1 | 1.98E−08 | 0.344 | 0.231 | 0.107 | 2.25E−04 |
| EIF4G3 | 2.24E−08 | 0.388 | 0.16 | 0.057 | 2.56E−04 |
| RNMT | 3.03E−08 | 0.290 | 0.244 | 0.12 | 3.45E−04 |
| CCNL1 | 3.62E−08 | 0.363 | 0.357 | 0.211 | 4.13E−04 |
| LUC7L3 | 3.73E−08 | 0.299 | 0.461 | 0.3 | 4.25E−04 |
| CDK9 | 4.76E−08 | 0.462 | 0.117 | 0.034 | 5.42E−04 |
| NFKBIZ | 6.09E−08 | 0.381 | 0.133 | 0.042 | 6.95E−04 |
| TMEM14C | 6.13E−08 | 0.312 | 0.485 | 0.325 | 6.99E−04 |
| BOLA3 | 8.69E−08 | 0.425 | 0.204 | 0.092 | 9.90E−04 |
| CCDC125 | 9.75E−08 | 0.324 | 0.141 | 0.048 | 1.11E−03 |
| GPT2 | 1.13E−07 | 0.282 | 0.127 | 0.039 | 1.29E−03 |
| MYCBP2 | 1.14E−07 | 0.263 | 0.192 | 0.089 | 1.30E−03 |
| BNIP3 | 1.32E−07 | 0.353 | 0.26 | 0.133 | 1.50E−03 |
| AGGF1 | 1.41E−07 | 0.485 | 0.124 | 0.039 | 1.60E−03 |
| MAP3K1 | 1.49E−07 | 0.380 | 0.163 | 0.063 | 1.70E−03 |
| CYP1B1 | 1.58E−07 | 0.841 | 0.195 | 0.13 | 1.80E−03 |
| KIAA1598 | 1.67E−07 | 0.264 | 0.223 | 0.111 | 1.90E−03 |
| LARP1B | 1.82E−07 | 0.253 | 0.209 | 0.099 | 2.08E−03 |
| MT-TT | 2.02E−07 | 0.251 | 0.103 | 0.031 | 2.30E−03 |
| KDM5B | 2.14E−07 | 0.337 | 0.222 | 0.105 | 2.44E−03 |
| AHCYL1 | 2.49E−07 | 0.251 | 0.247 | 0.127 | 2.84E−03 |
| USP47 | 2.81E−07 | 0.255 | 0.219 | 0.105 | 3.21E−03 |
| ESF1 | 2.83E−07 | 0.558 | 0.25 | 0.139 | 3.23E−03 |
| PYCARD | 3.31E−07 | 0.255 | 0.114 | 0.037 | 3.77E−03 |
| ARIH1 | 3.81E−07 | 0.320 | 0.173 | 0.072 | 4.35E−03 |
| SORL1 | 4.38E−07 | 1.057 | 0.12 | 0.042 | 4.99E−03 |
| RPLP2 | 4.76E−07 | 0.251 | 0.927 | 0.852 | 5.43E−03 |
| ODF2L | 4.99E−07 | 0.310 | 0.277 | 0.152 | 5.69E−03 |
| ARHGAP35 | 5.47E−07 | 0.252 | 0.239 | 0.126 | 6.23E−03 |
| RN7SL2 | 5.49E−07 | 0.350 | 0.177 | 0.076 | 6.26E−03 |
| HECTD1 | 7.23E−07 | 0.342 | 0.23 | 0.115 | 8.24E−03 |
| PNRC1 | 9.21E−07 | 0.299 | 0.236 | 0.123 | 1.05E−02 |
| EAPP | 9.42E−07 | 0.327 | 0.163 | 0.069 | 1.07E−02 |
| APOL6 | 1.10E−06 | 0.595 | 0.206 | 0.104 | 1.26E−02 |
| MYB | 1.34E−06 | 0.405 | 0.106 | 0.041 | 1.53E−02 |
| CITED2 | 1.44E−06 | 0.364 | 0.258 | 0.139 | 1.65E−02 |
| KIF16B | 2.66E−06 | 0.354 | 0.157 | 0.066 | 3.04E−02 |
| SAMD9L | 3.25E−06 | 0.549 | 0.106 | 0.041 | 3.71E−02 |
| NCOA7 | 3.73E−06 | 0.483 | 0.284 | 0.171 | 4.25E−02 |

Differential expression between the pre-treatment cells of responders (up) and non-responders (dn) and differential expression results between post-treatment cells of responders (up) and non-responders (dn). Interestingly, genes such as B32M and MIIC machinery is upregulated in non-responders pre-treatment, but shifts to responders post-treatment.

TABLE 4
Tcell_TvUT

| | | | | |
|---|---|---|---|---|
| p_val | avg_logFC | pct.1 | pct.2 | p_val_adj |

CSF.f.tcell.set.TvUT.dn.1

| | | | | | |
|---|---|---|---|---|---|
| MTRNR2L12 | 4.02E−68 | −1.972 | 0.228 | 0.704 | 3.22E−64 |
| MTRNR2L8 | 2.21E−50 | −1.496 | 0.438 | 0.756 | 1.78E−46 |
| MTRNR2L5 | 8.16E−37 | −0.903 | 0.068 | 0.444 | 6.55E−33 |
| RPSA | 9.49E−32 | −0.832 | 0.432 | 0.733 | 7.62E−28 |
| MTRNR2L7 | 7.54E−31 | −0.883 | 0.075 | 0.474 | 6.05E−27 |
| RPL10 | 4.68E−30 | −0.943 | 0.505 | 0.778 | 3.76E−26 |
| EEF1A1 | 1.87E−29 | −0.843 | 0.706 | 0.919 | 1.50E−25 |
| MTRNR2L13 | 5.76E−25 | −0.737 | 0.079 | 0.385 | 4.62E−21 |
| RPL22 | 2.19E−24 | −0.809 | 0.378 | 0.704 | 1.76E−20 |
| MTRNR2L2 | 1.41E−23 | −1.019 | 0.403 | 0.704 | 1.13E−19 |
| RPS27 | 1.00E−17 | −0.716 | 0.765 | 0.933 | 8.03E−14 |
| TIPIN | 1.53E−17 | −0.622 | 0.19 | 0.533 | 1.23E−13 |
| RPL18A | 1.31E−16 | −0.663 | 0.291 | 0.585 | 1.05E−12 |
| RNA28S5 | 2.16E−16 | −0.734 | 0.508 | 0.726 | 1.74E−12 |
| RPL3 | 3.97E−16 | −0.646 | 0.706 | 0.911 | 3.19E−12 |
| MT-ND2 | 1.82E−15 | −0.710 | 0.689 | 0.815 | 1.46E−11 |
| RPS2 | 3.47E−14 | −0.571 | 0.703 | 0.874 | 2.78E−10 |
| SNHG5 | 6.00E−14 | −0.542 | 0.526 | 0.822 | 4.81E−10 |
| EIF4B | 4.81E−11 | −0.479 | 0.262 | 0.519 | 3.86E−07 |
| MT-ATP6 | 8.08E−11 | −0.510 | 0.545 | 0.763 | 6.48E−07 |
| ZFP36L2 | 4.01E−10 | −0.564 | 0.473 | 0.637 | 3.21E−06 |
| RBM3 | 4.50E−10 | −0.452 | 0.352 | 0.526 | 3.61E−06 |
| RPS3A | 1.12E−09 | −0.513 | 0.475 | 0.711 | 8.97E−06 |
| MT-CO3 | 1.80E−09 | −0.560 | 0.71 | 0.852 | 1.44E−05 |
| CTC-338M12.5 | 4.24E−09 | −0.290 | 0.011 | 0.126 | 3.40E−05 |
| ID2 | 4.84E−09 | −0.622 | 0.298 | 0.481 | 3.89E−05 |
| RPL23A | 1.02E−08 | −0.364 | 0.77 | 0.956 | 8.20E−05 |
| JUNB | 1.90E−08 | −0.492 | 0.225 | 0.385 | 1.52E−04 |
| HSP90AB1 | 2.08E−08 | −0.498 | 0.452 | 0.659 | 1.67E−04 |
| GOLGA4 | 2.31E−08 | −0.505 | 0.145 | 0.274 | 1.86E−04 |
| MT-CO2 | 4.15E−08 | −0.452 | 0.515 | 0.741 | 3.33E−04 |
| FTH1 | 5.40E−08 | −0.583 | 0.465 | 0.622 | 4.34E−04 |
| TMSB4X | 6.23E−08 | −0.337 | 0.953 | 0.993 | 5.00E−04 |
| AL353147.1 | 7.34E−08 | −0.358 | 0.074 | 0.244 | 5.89E−04 |
| THUMPD1 | 1.53E−07 | −0.368 | 0.147 | 0.207 | 1.23E−03 |
| RPS7 | 1.90E−07 | −0.411 | 0.572 | 0.778 | 1.53E−03 |
| CCL4 | 2.84E−07 | −0.387 | 0.143 | 0.104 | 2.28E−03 |
| BCL2 | 2.97E−07 | −0.435 | 0.104 | 0.274 | 2.38E−03 |
| MT-ATP8 | 3.16E−07 | −0.362 | 0.227 | 0.444 | 2.54E−03 |
| ANKRD12 | 5.71E−07 | −0.385 | 0.404 | 0.459 | 4.58E−03 |
| PPIA | 7.14E−07 | −0.335 | 0.216 | 0.43 | 5.73E−03 |
| PABPC3 | 9.41E−07 | −0.361 | 0.162 | 0.37 | 7.55E−03 |
| RPS28 | 1.49E−06 | −0.376 | 0.754 | 0.896 | 1.19E−02 |
| GSTP1 | 1.92E−06 | −0.398 | 0.195 | 0.296 | 1.54E−02 |
| MT-CYB | 3.12E−06 | −0.454 | 0.623 | 0.733 | 2.50E−02 |
| RPL29 | 4.05E−06 | −0.371 | 0.483 | 0.63 | 3.25E−02 |
| SNORD89 | 5.22E−06 | −0.396 | 0.085 | 0.185 | 4.19E−02 |
| TPM3 | 6.03E−06 | −0.349 | 0.567 | 0.585 | 4.83E−02 |

CSF.f.tcell.set.TvUT.up.1

| | | | | | |
|---|---|---|---|---|---|
| B2M | 3.88E−33 | 0.572 | 1 | 1 | 3.12E−29 |
| RPL30 | 2.72E−20 | 0.695 | 0.999 | 0.993 | 2.19E−16 |
| HLA-DRA | 4.57E−17 | 0.839 | 0.405 | 0.089 | 3.66E−13 |
| FAU | 3.20E−15 | 0.559 | 0.968 | 0.926 | 2.57E−11 |
| RPL27 | 5.27E−14 | 0.606 | 0.987 | 0.97 | 4.22E−10 |
| RPS16 | 5.49E−13 | 0.572 | 0.98 | 0.985 | 4.41E−09 |
| ISG15 | 2.07E−12 | 1.009 | 0.429 | 0.17 | 1.66E−08 |
| RPS12 | 7.86E−12 | 0.490 | 0.998 | 0.993 | 6.31E−08 |
| STAT1 | 4.00E−11 | 0.822 | 0.446 | 0.2 | 3.21E−07 |
| IFI6 | 5.30E−11 | 0.661 | 0.331 | 0.163 | 4.25E−07 |
| MX1 | 1.33E−10 | 0.681 | 0.299 | 0.104 | 1.07E−06 |
| IFIT1 | 3.00E−10 | 0.582 | 0.203 | 0.015 | 2.41E−06 |
| HERC5 | 5.81E−10 | 0.563 | 0.191 | 0.007 | 4.66E−06 |
| RPS29 | 1.07E−09 | 0.472 | 1 | 0.993 | 8.57E−06 |
| RPS24 | 2.55E−09 | 0.419 | 0.984 | 0.933 | 2.05E−05 |
| CD74 | 4.11E−09 | 0.688 | 0.579 | 0.356 | 3.29E−05 |
| HLA-DRB1 | 4.51E−09 | 0.594 | 0.348 | 0.111 | 3.62E−05 |
| RPS21 | 5.59E−09 | 0.499 | 0.986 | 0.963 | 4.48E−05 |
| RPL39 | 5.74E−09 | 0.612 | 0.968 | 0.948 | 4.61E−05 |
| XAF1 | 6.14E−09 | 0.737 | 0.478 | 0.259 | 4.93E−05 |
| PSME1 | 6.23E−09 | 0.525 | 0.626 | 0.489 | 5.00E−05 |
| CCL5 | 1.02E−08 | 0.854 | 0.611 | 0.644 | 8.22E−05 |
| RPS11 | 1.14E−08 | 0.330 | 0.98 | 0.985 | 9.15E−05 |
| HLA-DPB1 | 1.24E−08 | 0.535 | 0.296 | 0.089 | 9.95E−05 |
| RPS15A | 1.38E−07 | 0.445 | 0.983 | 0.985 | 1.11E−03 |
| ISG20 | 1.70E−07 | 0.479 | 0.378 | 0.23 | 1.36E−03 |
| RSAD2 | 2.00E−07 | 0.499 | 0.184 | 0.022 | 1.60E−03 |
| EPSTI1 | 3.29E−07 | 0.504 | 0.316 | 0.104 | 2.64E−03 |
| GBP5 | 4.27E−07 | 0.618 | 0.423 | 0.267 | 3.42E−03 |
| IFI44L | 5.18E−07 | 0.512 | 0.201 | 0.037 | 4.16E−03 |
| COMMD6 | 7.86E−07 | 0.457 | 0.586 | 0.415 | 6.31E−03 |
| SAMD9L | 9.44E−07 | 0.566 | 0.379 | 0.163 | 7.58E−03 |
| SNRPD2 | 1.02E−06 | 0.402 | 0.664 | 0.452 | 8.22E−03 |
| RPL35A | 1.17E−06 | 0.375 | 0.995 | 0.978 | 9.35E−03 |
| MT2A | 1.35E−06 | 0.520 | 0.36 | 0.17 | 1.09E−02 |
| IFIT3 | 1.93E−06 | 0.502 | 0.173 | 0.03 | 1.55E−02 |
| UBB | 3.81E−06 | 0.448 | 0.736 | 0.585 | 3.05E−02 |
| OAS1 | 3.95E−06 | 0.329 | 0.188 | 0.059 | 3.17E−02 |
| CD52 | 4.44E−06 | 0.437 | 0.927 | 0.83 | 3.56E−02 |
| ZFAS1 | 4.63E−06 | 0.475 | 0.568 | 0.422 | 3.71E−02 |
| IFI16 | 5.59E−06 | 0.463 | 0.533 | 0.311 | 4.49E−02 |
| ATP5E | 5.86E−06 | 0.277 | 0.818 | 0.659 | 4.70E−02 |

Differential expression results between treated and untreated T cell populations.

TABLE 5
Tcell_RvNR

| | | | | |
|---|---|---|---|---|
| p_val | avg_logFC | pct.1 | pct.2 | p_val_adj |

CSF.f.tcell.set.RvNR.dn.1

| | | | | | |
|---|---|---|---|---|---|
| SNHG5 | 1.21E−53 | −1.202 | 0.36 | 0.824 | 9.73E−50 |
| RPS2 | 3.04E−42 | −0.927 | 0.6 | 0.886 | 2.44E−38 |
| RNA28S5 | 2.75E−41 | −1.061 | 0.417 | 0.818 | 2.21E−37 |
| RPS27A | 6.81E−31 | −0.780 | 0.944 | 0.972 | 5.47E−27 |
| RPL21 | 2.14E−29 | −0.666 | 0.099 | 0.477 | 1.72E−25 |
| RPL41 | 3.56E−27 | −0.713 | 0.895 | 0.983 | 2.86E−23 |
| RPS18 | 3.19E−26 | −0.779 | 0.794 | 0.989 | 2.56E−22 |
| PABPC1 | 4.13E−26 | −0.810 | 0.831 | 0.943 | 3.32E−22 |
| RPL26 | 3.02E−25 | −0.711 | 0.954 | 0.972 | 2.42E−21 |
| RPS13 | 4.61E−25 | −0.784 | 0.918 | 0.977 | 3.69E−21 |
| RPL5 | 1.10E−24 | −0.712 | 0.857 | 0.977 | 8.82E−21 |
| RPL9 | 1.44E−24 | −0.741 | 0.415 | 0.722 | 1.15E−20 |
| LTB | 6.05E−24 | −0.963 | 0.155 | 0.5 | 4.86E−20 |
| RPS29 | 8.17E−24 | −0.546 | 1 | 1 | 6.55E−20 |
| RPS25 | 1.21E−23 | −0.779 | 0.742 | 0.938 | 9.72E−20 |
| EEF1B2 | 1.19E−21 | −0.684 | 0.509 | 0.767 | 9.56E−18 |
| RPL15 | 3.56E−20 | −0.575 | 0.888 | 0.943 | 2.86E−16 |
| TMSB4X | 5.07E−19 | −0.745 | 0.914 | 0.994 | 4.07E−15 |
| RPS14 | 5.96E−19 | −0.616 | 0.96 | 1 | 4.78E−15 |
| RPL23 | 8.73E−19 | −0.650 | 0.903 | 0.949 | 7.01E−15 |
| RPL4 | 1.70E−18 | −0.612 | 0.639 | 0.858 | 1.37E−14 |
| RPL23A | 6.48E−18 | −0.622 | 0.617 | 0.881 | 5.20E−14 |
| RPSA | 1.20E−17 | −0.489 | 0.298 | 0.636 | 9.60E−14 |
| RPLP0 | 1.26E−17 | −0.626 | 0.742 | 0.875 | 1.01E−13 |
| RPL35A | 4.96E−17 | −0.546 | 0.991 | 1 | 3.98E−13 |
| RPS27 | 1.05E−16 | −0.609 | 0.656 | 0.909 | 8.45E−13 |
| MT-CO3 | 4.98E−16 | −0.605 | 0.655 | 0.852 | 4.00E−12 |
| RPL31 | 7.12E−16 | −0.513 | 0.983 | 1 | 5.72E−12 |
| RPS15A | 1.82E−15 | −0.586 | 0.97 | 0.989 | 1.46E−11 |
| RPS8 | 1.87E−15 | −0.521 | 0.98 | 0.989 | 1.50E−11 |
| RPS6 | 3.06E−15 | −0.470 | 0.993 | 1 | 2.45E−11 |
| MT-ND6 | 1.20E−14 | −0.465 | 0.09 | 0.301 | 9.61E−11 |
| RPL6 | 2.18E−14 | −0.527 | 0.501 | 0.733 | 1.75E−10 |
| IL7R | 4.02E−14 | −0.405 | 0.334 | 0.659 | 3.22E−10 |
| RPL34 | 4.30E−14 | −0.518 | 0.987 | 0.983 | 3.45E−10 |
| RPL11 | 4.31E−14 | −0.458 | 0.991 | 0.977 | 3.46E−10 |
| RPL30 | 7.84E−14 | −0.463 | 0.999 | 1 | 6.29E−10 |
| RN7SL1 | 1.54E−13 | −0.523 | 0.026 | 0.148 | 1.23E−09 |
| MT-ATP6 | 1.18E−11 | −0.433 | 0.499 | 0.71 | 9.48E−08 |
| RPL35 | 2.78E−11 | −0.445 | 0.913 | 0.972 | 2.23E−07 |
| RPLP2 | 3.23E−11 | −0.451 | 0.974 | 0.994 | 2.59E−07 |
| MT-ND1 | 3.76E−11 | −0.534 | 0.258 | 0.494 | 3.02E−07 |
| ATP5EP2 | 8.23E−11 | −0.442 | 0.299 | 0.54 | 6.60E−07 |
| TPT1 | 1.56E−10 | −0.419 | 0.991 | 0.983 | 1.25E−06 |
| RPL12 | 2.80E−10 | −0.394 | 0.835 | 0.932 | 2.25E−06 |
| RPS3A | 6.28E−10 | −0.416 | 0.311 | 0.562 | 5.03E−06 |
| HEBP2 | 7.85E−10 | −0.317 | 0.059 | 0.142 | 6.30E−06 |
| RPL37 | 1.01E−09 | −0.390 | 0.979 | 0.966 | 8.07E−06 |
| RPS7 | 1.66E−09 | −0.404 | 0.387 | 0.648 | 1.33E−05 |
| RPS3 | 3.51E−09 | −0.356 | 0.92 | 0.977 | 2.81E−05 |
| EEF1A1 | 3.78E−09 | −0.406 | 0.648 | 0.835 | 3.03E−05 |
| MT-ND4 | 4.89E−09 | −0.501 | 0.628 | 0.812 | 3.93E−05 |
| RPL22 | 5.36E−09 | −0.396 | 0.301 | 0.506 | 4.30E−05 |
| RPL7A | 7.17E−09 | −0.371 | 0.719 | 0.841 | 5.75E−05 |
| RPL32 | 8.28E−09 | −0.408 | 0.953 | 0.972 | 6.64E−05 |
| GLTSCR2 | 8.54E−09 | −0.415 | 0.494 | 0.659 | 6.85E−05 |
| RPL10A | 9.35E−09 | −0.416 | 0.66 | 0.835 | 7.50E−05 |
| FOS | 1.44E−08 | −0.335 | 0.044 | 0.131 | 1.16E−04 |
| RPS20 | 3.24E−08 | −0.332 | 0.98 | 0.972 | 2.60E−04 |
| NACA2 | 4.57E−08 | −0.317 | 0.178 | 0.369 | 3.66E−04 |
| BRD2 | 5.85E−08 | −0.367 | 0.256 | 0.466 | 4.69E−04 |
| RPL39 | 6.33E−08 | −0.418 | 0.958 | 0.966 | 5.08E−04 |
| RPS28 | 1.10E−07 | −0.379 | 0.66 | 0.812 | 8.82E−04 |
| MT-CO1 | 1.52E−07 | −0.373 | 0.752 | 0.875 | 1.22E−03 |
| PABPC4 | 1.52E−07 | −0.267 | 0.053 | 0.205 | 1.22E−03 |
| NBEAL1 | 2.11E−07 | −0.352 | 0.172 | 0.369 | 1.69E−03 |
| DYNC1I2 | 3.59E−07 | −0.312 | 0.082 | 0.216 | 2.88E−03 |
| PTMA | 3.68E−07 | −0.342 | 0.827 | 0.886 | 2.95E−03 |
| RPL27A | 4.18E−07 | −0.330 | 0.986 | 1 | 3.35E−03 |
| TOMM7 | 4.55E−07 | −0.361 | 0.623 | 0.773 | 3.65E−03 |
| TIPIN | 4.62E−07 | −0.294 | 0.129 | 0.301 | 3.71E−03 |
| TCF7 | 5.09E−07 | −0.345 | 0.097 | 0.216 | 4.09E−03 |
| RPS23 | 5.47E−07 | −0.368 | 0.95 | 0.977 | 4.39E−03 |
| TMEM70 | 5.90E−07 | −0.326 | 0.264 | 0.472 | 4.73E−03 |
| MT-CYB | 6.12E−07 | −0.412 | 0.534 | 0.722 | 4.91E−03 |
| BAZ1B | 7.27E−07 | −0.263 | 0.155 | 0.267 | 5.83E−03 |
| TMA7 | 8.47E−07 | −0.344 | 0.357 | 0.517 | 6.80E−03 |
| NFKBIA | 1.88E−06 | −0.345 | 0.11 | 0.244 | 1.51E−02 |
| CD52 | 2.56E−06 | −0.484 | 0.888 | 0.932 | 2.05E−02 |
| GADD45B | 2.65E−06 | −0.252 | 0.087 | 0.244 | 2.12E−02 |
| DGKA | 2.82E−06 | −0.285 | 0.128 | 0.153 | 2.26E−02 |
| RPS10 | 3.29E−06 | −0.288 | 0.46 | 0.67 | 2.64E−02 |
| BCCIP | 4.56E−06 | −0.276 | 0.069 | 0.102 | 3.66E−02 |
| ZNF90 | 4.62E−06 | −0.287 | 0.152 | 0.324 | 3.71E−02 |

CSF.f.tcell.set.RvNR.dn.1

| | | | | | |
|---|---|---|---|---|---|
| MT-RNR2 | 5.08E−88 | 0.876 | 1 | 1 | 4.07E−84 |
| MTRNR2L1 | 1.46E−44 | 1.649 | 0.874 | 0.58 | 1.17E−40 |
| STAT1 | 1.94E−33 | 1.093 | 0.713 | 0.233 | 1.55E−29 |
| ISG15 | 4.18E−24 | 1.124 | 0.669 | 0.307 | 3.35E−20 |
| XAF1 | 6.61E−22 | 0.857 | 0.718 | 0.358 | 5.30E−18 |
| MT-RNR1 | 6.90E−21 | 1.258 | 0.954 | 0.903 | 5.54E−17 |
| GBP5 | 9.04E−21 | 0.816 | 0.643 | 0.244 | 7.25E−17 |
| CCL5 | 4.29E−18 | 1.202 | 0.746 | 0.438 | 3.44E−14 |
| IFIT3 | 1.37E−17 | 0.580 | 0.312 | 0.034 | 1.10E−13 |
| IFITM1 | 4.29E−17 | 0.789 | 0.446 | 0.239 | 3.44E−13 |
| SAMD9 | 6.10E−17 | 0.870 | 0.605 | 0.273 | 4.90E−13 |
| MT2A | 6.91E−16 | 0.717 | 0.514 | 0.205 | 5.54E−12 |
| MX1 | 2.34E−15 | 0.670 | 0.491 | 0.159 | 1.88E−11 |
| CST7 | 3.81E−15 | 0.689 | 0.546 | 0.222 | 3.06E−11 |
| B2M | 8.31E−15 | 0.343 | 1 | 1 | 6.67E−11 |
| PSME1 | 9.71E−15 | 0.633 | 0.741 | 0.517 | 7.79E−11 |
| SLFN5 | 1.22E−14 | 0.694 | 0.792 | 0.58 | 9.79E−11 |
| IFI16 | 5.26E−14 | 0.660 | 0.696 | 0.455 | 4.22E−10 |
| IFIT1 | 7.73E−14 | 0.491 | 0.358 | 0.091 | 6.21E−10 |
| IFI6 | 6.53E−13 | 0.599 | 0.527 | 0.222 | 5.24E−09 |
| CD74 | 1.33E−12 | 0.586 | 0.67 | 0.375 | 1.07E−08 |
| HERC5 | 2.99E−12 | 0.529 | 0.311 | 0.062 | 2.40E−08 |
| GZMA | 4.55E−12 | 0.485 | 0.702 | 0.398 | 3.65E−08 |
| GBP1 | 3.32E−11 | 0.652 | 0.51 | 0.256 | 2.66E−07 |
| HLA-A | 3.43E−11 | 0.470 | 0.914 | 0.886 | 2.75E−07 |
| SAMD9L | 4.81E−11 | 0.469 | 0.574 | 0.278 | 3.86E−07 |
| ISG20 | 7.14E−11 | 0.479 | 0.506 | 0.244 | 5.73E−07 |
| RARRES3 | 8.98E−11 | 0.560 | 0.497 | 0.29 | 7.20E−07 |
| PTPRCAP | 3.46E−10 | 0.495 | 0.754 | 0.625 | 2.78E−06 |
| HIST1H1D | 4.16E−10 | 0.366 | 0.371 | 0.148 | 3.34E−06 |
| HLA-DPA1 | 6.77E−10 | 0.556 | 0.403 | 0.182 | 5.43E−06 |
| RSAD2 | 6.85E−10 | 0.364 | 0.305 | 0.091 | 5.49E−06 |
| EIF2AK2 | 9.94E−10 | 0.425 | 0.451 | 0.199 | 7.98E−06 |
| GIMAP4 | 1.16E−09 | 0.536 | 0.645 | 0.403 | 9.31E−06 |
| PARP9 | 1.21E−09 | 0.491 | 0.383 | 0.148 | 9.72E−06 |
| SRSF5 | 1.26E−09 | 0.470 | 0.738 | 0.506 | 1.01E−05 |
| HLA-DRB1 | 1.57E−09 | 0.556 | 0.451 | 0.21 | 1.26E−05 |
| IFIT2 | 2.95E−09 | 0.467 | 0.208 | 0.034 | 2.37E−05 |
| CD8A | 5.93E−09 | 0.497 | 0.268 | 0.074 | 4.76E−05 |
| GZMK | 9.12E−09 | 0.664 | 0.507 | 0.273 | 7.32E−05 |
| NMI | 1.19E−08 | 0.421 | 0.342 | 0.125 | 9.53E−05 |
| PSMB9 | 1.69E−08 | 0.444 | 0.428 | 0.21 | 1.35E−04 |
| OAS2 | 1.75E−08 | 0.316 | 0.331 | 0.119 | 1.40E−04 |
| MTRNR2L2 | 2.16E−08 | 0.455 | 0.487 | 0.318 | 1.73E−04 |
| PPP2R5C | 4.67E−08 | 0.466 | 0.493 | 0.256 | 3.75E−04 |
| PYHIN1 | 4.73E−08 | 0.427 | 0.398 | 0.17 | 3.79E−04 |
| APOL6 | 7.15E−08 | 0.475 | 0.497 | 0.295 | 5.74E−04 |
| NT5C3A | 9.08E−08 | 0.496 | 0.312 | 0.125 | 7.28E−04 |
| EPSTI1 | 1.21E−07 | 0.416 | 0.463 | 0.227 | 9.72E−04 |
| NAA50 | 1.27E−07 | 0.265 | 0.216 | 0.057 | 1.02E−03 |
| GZMH | 1.29E−07 | 0.441 | 0.264 | 0.074 | 1.03E−03 |
| CLEC2D | 2.31E−07 | 0.457 | 0.394 | 0.188 | 1.85E−03 |
| HLA-DRA | 2.61E−07 | 0.466 | 0.41 | 0.199 | 2.09E−03 |
| IFIH1 | 4.63E−07 | 0.397 | 0.295 | 0.108 | 3.71E−03 |
| DDX60 | 6.99E−07 | 0.357 | 0.268 | 0.085 | 5.61E−03 |
| SRGN | 7.45E−07 | 0.419 | 0.761 | 0.562 | 5.98E−03 |
| RGS1 | 8.02E−07 | 0.675 | 0.443 | 0.267 | 6.44E−03 |
| EVL | 1.03E−06 | 0.358 | 0.484 | 0.295 | 8.28E−03 |
| TIGIT | 1.28E−06 | 0.454 | 0.36 | 0.165 | 1.03E−02 |
| RNF213 | 1.30E−06 | 0.409 | 0.656 | 0.455 | 1.04E−02 |
| HCST | 1.32E−06 | 0.420 | 0.46 | 0.307 | 1.06E−02 |
| MX2 | 1.40E−06 | 0.458 | 0.289 | 0.119 | 1.13E−02 |
| UBE2L6 | 1.50E−06 | 0.338 | 0.378 | 0.188 | 1.21E−02 |
| MT-CO2 | 2.90E−06 | 0.430 | 0.5 | 0.369 | 2.33E−02 |
| F2R | 2.92E−06 | 0.335 | 0.202 | 0.051 | 2.34E−02 |
| DDX58 | 3.21E−06 | 0.281 | 0.202 | 0.057 | 2.57E−02 |
| SP110 | 3.62E−06 | 0.277 | 0.43 | 0.244 | 2.91E−02 |
| LYST | 3.80E−06 | 0.323 | 0.315 | 0.131 | 3.05E−02 |
| HIST1H4C | 4.40E−06 | 0.377 | 0.481 | 0.267 | 3.53E−02 |
| GBP4 | 4.42E−06 | 0.314 | 0.364 | 0.188 | 3.55E−02 |
| UBB | 4.89E−06 | 0.289 | 0.828 | 0.705 | 3.92E−02 |

Differential expression results between responder and non-responder T cell populations. Applicants see classic up regulation of interferon associated genes here. STAT1, all the IFI's, as well as NMI and IFIH1, which is important because these genes bind each other when BATF is not present (which IFIH1 would usually bind). BATF has previously been shown to disrupt T cell recruitment so it is consistent that its competitor would be upregulated in responders. Applicants also see that cytotoxic genes (CD8A, GZMK, etc.) are upregulated.

TABLE 6
Mac_TvUT

| | | | | |
|---|---|---|---|---|
| p_val | avg_logFC | pct.1 | pct.2 | p_val_adj |

CSF.f.mac.set.TvUT.dn.1

| | | | | | |
|---|---|---|---|---|---|
| MTRNR2L2 | 9.30E−18 | −1.915346135 | 0.365 | 0.545 | 7.46E−14 |
| MTRNR2L6 | 9.28E−12 | −1.490085779 | 0.219 | 0.455 | 7.45E−08 |
| MTRNR2L12 | 1.71E−11 | −1.662074689 | 0.36 | 0.591 | 1.38E−07 |
| CTC- | 3.75E−09 | −2.121783336 | 0.033 | 0.318 | 3.01E−05 |
| 338M12.5 | | | | | |
| HSPA1A | 1.09E−08 | −1.220092594 | 0.16 | 0.182 | 8.72E−05 |
| MTRNR2L8 | 3.87E−07 | −1.424082267 | 0.438 | 0.5 | 0.003101352 |
| MTRNR2L1 | 6.16E−07 | −0.973007029 | 0.714 | 0.795 | 0.004938416 |
| EGR1 | 4.88E−06 | −0.860830345 | 0.066 | 0.25 | 0.039132875 |

CSF.f.mac.set.TvUT.up.1

| | | | | | |
|---|---|---|---|---|---|
| SIVA1 | 3.53E−07 | 0.6174436 | 0.242 | 0.045 | 0.002835315 |
| RPL18A | 1.53E−06 | 0.840190359 | 0.357 | 0.045 | 0.012309552 |

Differential expression results between treated and untreated macrophage populations. Both of them have few genes because there were almost no pre-treatment macrophages. This was an unexpected finding of the study.

TABLE 7
Mac_RvNR

| | | | | |
|---|---|---|---|---|
| p_val | avg_logFC | pct.1 | pct.2 | p_val_adj |

CSF.f.mac.set.RvNR.dn.1

| | | | | | |
|---|---|---|---|---|---|
| SPP1 | 1.70E−24 | −2.890 | 0.095 | 0.545 | 1.36E−20 |
| MTRNR2L12 | 6.54E−14 | −1.197 | 0.206 | 0.507 | 5.25E−10 |
| SNHG5 | 1.66E−11 | −0.888 | 0.492 | 0.615 | 1.34E−07 |
| ARF1 | 1.88E−11 | −0.687 | 0.513 | 0.493 | 1.51E−07 |
| RPL23 | 3.61E−11 | −0.331 | 0.854 | 0.812 | 2.90E−07 |
| FAM49B | 2.01E−10 | −0.337 | 0.538 | 0.404 | 1.61E−06 |
| MT-CO1 | 5.02E−10 | −0.622 | 0.799 | 0.69 | 4.03E−06 |
| BTG1 | 1.02E−09 | −0.750 | 0.518 | 0.582 | 8.15E−06 |
| SH3BGRL | 2.02E−09 | −0.375 | 0.698 | 0.587 | 1.62E−05 |
| PRKDC | 5.20E−09 | −0.679 | 0.161 | 0.183 | 4.17E−05 |
| PPT1 | 6.93E−09 | −0.579 | 0.467 | 0.474 | 5.56E−05 |
| OAS2 | 7.24E−09 | −0.294 | 0.518 | 0.357 | 5.81E−05 |
| RNA28S5 | 1.05E−08 | −0.723 | 0.623 | 0.817 | 8.45E−05 |
| PLXDC2 | 1.06E−08 | −0.958 | 0.352 | 0.474 | 8.47E−05 |
| APOE | 2.31E−08 | −0.322 | 0.176 | 0.432 | 1.85E−04 |
| UVRAG | 3.41E−08 | −0.289 | 0.241 | 0.15 | 2.74E−04 |
| LHFPL2 | 4.11E−08 | −1.143 | 0.136 | 0.3 | 3.30E−04 |
| DUSP6 | 4.19E−08 | −0.339 | 0.327 | 0.211 | 3.37E−04 |
| LGALS3 | 5.15E−08 | −0.850 | 0.387 | 0.352 | 4.13E−04 |
| DNTTIP2 | 6.38E−08 | −0.303 | 0.317 | 0.239 | 5.12E−04 |
| TLN1 | 7.08E−08 | −0.620 | 0.668 | 0.728 | 5.68E−04 |
| WAPAL | 7.09E−08 | −0.508 | 0.236 | 0.211 | 5.69E−04 |
| FKBP5 | 9.75E−08 | −0.705 | 0.296 | 0.39 | 7.82E−04 |
| MTRNR2L8 | 1.03E−07 | −0.671 | 0.347 | 0.446 | 8.28E−04 |
| MSR1 | 1.14E−07 | −0.770 | 0.528 | 0.554 | 9.13E−04 |
| TNIP1 | 1.47E−07 | −0.371 | 0.201 | 0.131 | 1.18E−03 |
| PKM | 1.58E−07 | −0.535 | 0.467 | 0.479 | 1.27E−03 |
| ABI1 | 1.60E−07 | −0.320 | 0.352 | 0.296 | 1.29E−03 |
| DARS | 1.66E−07 | −0.580 | 0.231 | 0.225 | 1.33E−03 |
| TSPAN31 | 1.97E−07 | −0.355 | 0.126 | 0.08 | 1.58E−03 |
| FAM129A | 2.05E−07 | −0.318 | 0.216 | 0.183 | 1.64E−03 |
| ID2 | 2.25E−07 | −0.559 | 0.422 | 0.371 | 1.81E−03 |
| SGK1 | 2.76E−07 | −1.038 | 0.216 | 0.31 | 2.22E−03 |
| FBP1 | 2.93E−07 | −1.164 | 0.146 | 0.254 | 2.35E−03 |
| GTF3A | 2.98E−07 | −0.302 | 0.422 | 0.319 | 2.39E−03 |
| GPRIN3 | 3.02E−07 | −0.786 | 0.161 | 0.244 | 2.42E−03 |
| CREG1 | 3.70E−07 | −0.743 | 0.266 | 0.31 | 2.97E−03 |
| SUGT1 | 4.22E−07 | −0.281 | 0.206 | 0.146 | 3.39E−03 |
| SPG20 | 4.31E−07 | −0.310 | 0.226 | 0.169 | 3.45E−03 |
| MT-CYB | 5.00E−07 | −0.656 | 0.578 | 0.573 | 4.01E−03 |
| MTRNR2L6 | 5.54E−07 | −0.604 | 0.216 | 0.338 | 4.45E−03 |
| RPS25 | 5.68E−07 | −0.526 | 0.588 | 0.695 | 4.56E−03 |
| ZFP36L2 | 6.25E−07 | −0.406 | 0.447 | 0.531 | 5.01E−03 |
| CCAR1 | 6.99E−07 | −0.273 | 0.276 | 0.202 | 5.61E−03 |
| CTC-338M12.5 | 7.24E−07 | −1.597 | 0.015 | 0.136 | 5.81E−03 |
| RAP1A | 7.64E−07 | −0.425 | 0.452 | 0.399 | 6.13E−03 |
| ATP6V1G1 | 7.74E−07 | −0.304 | 0.518 | 0.408 | 6.21E−03 |
| PDS5B | 8.64E−07 | −0.439 | 0.136 | 0.108 | 6.94E−03 |
| BST2 | 9.72E−07 | −0.377 | 0.523 | 0.446 | 7.80E−03 |
| MACF1 | 1.01E−06 | −0.756 | 0.271 | 0.338 | 8.11E−03 |
| FTH1 | 1.01E−06 | −0.632 | 0.824 | 0.92 | 8.12E−03 |
| M6PR | 1.12E−06 | −0.288 | 0.372 | 0.282 | 8.96E−03 |
| APLP2 | 1.15E−06 | −0.513 | 0.658 | 0.629 | 9.21E−03 |
| CYTH1 | 1.16E−06 | −0.449 | 0.236 | 0.254 | 9.33E−03 |
| C3orf58 | 1.26E−06 | −0.576 | 0.161 | 0.146 | 1.01E−02 |
| CAP1 | 1.33E−06 | −0.460 | 0.688 | 0.653 | 1.07E−02 |
| NUDT21 | 1.46E−06 | −0.340 | 0.151 | 0.131 | 1.17E−02 |
| LPGAT1 | 1.53E−06 | −0.320 | 0.151 | 0.131 | 1.23E−02 |
| CTSH | 1.59E−06 | −0.251 | 0.759 | 0.629 | 1.27E−02 |
| AHNAK | 1.84E−06 | −0.825 | 0.226 | 0.343 | 1.48E−02 |
| CD84 | 2.00E−06 | −0.489 | 0.276 | 0.282 | 1.61E−02 |
| AKAP9 | 2.05E−06 | −0.258 | 0.432 | 0.31 | 1.65E−02 |
| RHOA | 2.09E−06 | −0.339 | 0.849 | 0.798 | 1.67E−02 |
| NCF2 | 2.30E−06 | −0.368 | 0.472 | 0.366 | 1.84E−02 |
| MYCBP2 | 2.39E−06 | −0.358 | 0.296 | 0.235 | 1.92E−02 |
| CLTC | 2.43E−06 | −0.588 | 0.317 | 0.357 | 1.95E−02 |
| RPS6KA3 | 2.54E−06 | −0.269 | 0.196 | 0.146 | 2.04E−02 |
| ZCCHC2 | 2.94E−06 | −0.339 | 0.191 | 0.136 | 2.36E−02 |
| SPEN | 3.18E−06 | −0.521 | 0.231 | 0.268 | 2.55E−02 |
| TRIP12 | 3.45E−06 | −0.653 | 0.271 | 0.343 | 2.77E−02 |
| MBNL1 | 3.45E−06 | −0.277 | 0.643 | 0.596 | 2.77E−02 |
| LAT2 | 3.67E−06 | −0.336 | 0.236 | 0.16 | 2.95E−02 |
| YWHAB | 3.68E−06 | −0.330 | 0.734 | 0.69 | 2.95E−02 |
| MTRNR2L2 | 3.94E−06 | −0.621 | 0.367 | 0.498 | 3.16E−02 |
| TREM2 | 4.06E−06 | −0.843 | 0.141 | 0.347 | 3.26E−02 |
| CAPZA2 | 4.08E−06 | −0.420 | 0.442 | 0.399 | 3.27E−02 |
| MYO5A | 4.11E−06 | −0.645 | 0.166 | 0.258 | 3.30E−02 |
| RNF149 | 4.43E−06 | −0.552 | 0.291 | 0.282 | 3.55E−02 |
| UBTF | 4.52E−06 | −0.384 | 0.151 | 0.174 | 3.63E−02 |
| MTRNR2L11 | 4.60E−06 | −0.266 | 0.181 | 0.207 | 3.69E−02 |
| LCP1 | 4.70E−06 | −0.435 | 0.734 | 0.714 | 3.77E−02 |
| LAPTM5 | 4.95E−06 | −0.322 | 0.794 | 0.775 | 3.97E−02 |
| TPM4 | 4.95E−06 | −0.534 | 0.492 | 0.592 | 3.97E−02 |
| RIPK2 | 5.06E−06 | −0.342 | 0.176 | 0.113 | 4.06E−02 |
| CYFIP1 | 5.07E−06 | −0.584 | 0.266 | 0.31 | 4.07E−02 |
| GNPDA1 | 5.33E−06 | −0.345 | 0.161 | 0.117 | 4.28E−02 |
| MLEC | 5.61E−06 | −0.425 | 0.261 | 0.225 | 4.50E−02 |
| PAFAH1B1 | 6.06E−06 | −0.355 | 0.261 | 0.207 | 4.86E−02 |
| CAB39 | 6.16E−06 | −0.405 | 0.151 | 0.178 | 4.94E−02 |

CSF.f.mac.set.RvNR.up.1

| | | | | | |
|---|---|---|---|---|---|
| RPS11 | 1.36E−29 | 0.324 | 0.985 | 0.775 | 1.09E−25 |
| RPS12 | 1.30E−27 | 0.346 | 0.995 | 0.911 | 1.04E−23 |
| RPL30 | 1.31E−27 | 0.397 | 0.97 | 0.883 | 1.05E−23 |
| B2M | 1.09E−24 | 0.784 | 1 | 0.995 | 8.73E−21 |
| TNFSF10 | 1.75E−22 | 1.151 | 0.764 | 0.305 | 1.41E−18 |
| RPL39 | 9.74E−21 | 0.527 | 0.945 | 0.756 | 7.81E−17 |
| RPL19 | 9.58E−19 | 0.506 | 0.965 | 0.892 | 7.69E−15 |
| IFITM3 | 2.61E−18 | 1.158 | 0.744 | 0.455 | 2.09E−14 |
| ISG15 | 2.71E−18 | 0.915 | 0.819 | 0.488 | 2.18E−14 |
| RPS21 | 3.45E−18 | 0.367 | 0.955 | 0.836 | 2.77E−14 |
| RPL28 | 6.91E−17 | 0.607 | 0.849 | 0.653 | 5.54E−13 |
| RPL38 | 1.07E−16 | 0.439 | 0.97 | 0.854 | 8.60E−13 |
| CD74 | 1.15E−16 | 0.747 | 0.975 | 0.925 | 9.21E−13 |
| CST3 | 1.45E−16 | 0.789 | 0.894 | 0.648 | 1.16E−12 |
| RPL27 | 2.99E−16 | 0.321 | 0.96 | 0.873 | 2.40E−12 |
| TMSB10 | 3.31E−16 | 0.445 | 0.99 | 0.953 | 2.65E−12 |
| HLA-DRB1 | 4.06E−16 | 0.886 | 0.95 | 0.822 | 3.25E−12 |
| RPS16 | 9.73E−16 | 0.282 | 0.93 | 0.85 | 7.81E−12 |
| FAU | 3.17E−15 | 0.307 | 0.955 | 0.779 | 2.54E−11 |
| VAMP5 | 5.05E−15 | 0.760 | 0.513 | 0.155 | 4.05E−11 |
| MTRNR2L1 | 8.15E−15 | 0.675 | 0.854 | 0.746 | 6.54E−11 |
| SLFN5 | 8.96E−15 | 0.780 | 0.558 | 0.192 | 7.19E−11 |
| GBP1 | 1.41E−14 | 0.685 | 0.628 | 0.263 | 1.13E−10 |
| RPS15 | 1.77E−14 | 0.342 | 0.799 | 0.577 | 1.42E−10 |
| IFITM1 | 2.08E−14 | 1.036 | 0.286 | 0.103 | 1.67E−10 |
| FCN1 | 2.28E−14 | 1.246 | 0.543 | 0.202 | 1.83E−10 |
| HLA-A | 5.84E−14 | 0.642 | 0.899 | 0.803 | 4.69E−10 |
| TYROBP | 7.21E−14 | 0.720 | 0.894 | 0.845 | 5.79E−10 |
| UBE2L6 | 3.02E−13 | 0.761 | 0.568 | 0.235 | 2.42E−09 |
| GBP4 | 3.32E−13 | 0.644 | 0.548 | 0.211 | 2.66E−09 |
| RPS4X | 4.15E−13 | 0.342 | 0.844 | 0.657 | 3.33E−09 |
| MT-ND3 | 1.93E−12 | 0.687 | 0.879 | 0.634 | 1.55E−08 |
| MT2A | 2.89E−12 | 1.075 | 0.513 | 0.207 | 2.32E−08 |
| IFITM2 | 3.40E−12 | 0.751 | 0.618 | 0.366 | 2.73E−08 |
| RPS19 | 4.53E−12 | 0.279 | 0.945 | 0.869 | 3.63E−08 |
| NT5C3A | 9.69E−12 | 0.779 | 0.593 | 0.258 | 7.77E−08 |
| TPT1 | 1.39E−11 | 0.281 | 0.99 | 0.958 | 1.11E−07 |
| FGL2 | 2.04E−11 | 0.629 | 0.824 | 0.601 | 1.64E−07 |
| PLAC8 | 2.39E−11 | 0.371 | 0.523 | 0.207 | 1.92E−07 |
| RPL32 | 3.29E−11 | 0.260 | 0.905 | 0.822 | 2.64E−07 |
| LAP3 | 3.41E−11 | 0.341 | 0.809 | 0.545 | 2.74E−07 |
| TNFSF13B | 4.21E−11 | 0.622 | 0.749 | 0.441 | 3.38E−07 |
| RPS9 | 6.73E−11 | 0.471 | 0.809 | 0.634 | 5.40E−07 |
| HLA-DPB1 | 9.47E−11 | 0.646 | 0.829 | 0.629 | 7.60E−07 |
| PSME2 | 1.09E−10 | 0.581 | 0.563 | 0.31 | 8.76E−07 |
| HLA-DPA1 | 1.93E−10 | 0.489 | 0.945 | 0.817 | 1.55E−06 |
| RPL18 | 2.30E−10 | 0.355 | 0.769 | 0.54 | 1.84E−06 |
| GBP5 | 2.58E−10 | 0.808 | 0.407 | 0.117 | 2.07E−06 |
| HLA-B | 3.13E−10 | 0.459 | 0.97 | 0.934 | 2.51E−06 |
| STAT1 | 3.92E−10 | 0.298 | 0.824 | 0.554 | 3.14E−06 |
| LY96 | 4.69E−10 | 0.742 | 0.568 | 0.258 | 3.76E−06 |
| IFI6 | 6.20E−10 | 0.271 | 0.799 | 0.498 | 4.97E−06 |
| GIMAP2 | 6.53E−10 | 0.356 | 0.327 | 0.085 | 5.24E−06 |
| UBA52 | 7.20E−10 | 0.368 | 0.859 | 0.657 | 5.78E−06 |
| IFIT2 | 8.01E−10 | 0.670 | 0.719 | 0.427 | 6.43E−06 |
| HLA-DRA | 1.49E−09 | 0.452 | 0.96 | 0.92 | 1.19E−05 |
| NMI | 1.54E−09 | 0.405 | 0.628 | 0.347 | 1.24E−05 |
| XAF1 | 1.93E−09 | 0.302 | 0.839 | 0.559 | 1.55E−05 |
| WSB1 | 2.29E−09 | 0.391 | 0.618 | 0.371 | 1.84E−05 |
| PPA1 | 2.42E−09 | 0.437 | 0.573 | 0.286 | 1.94E−05 |
| CCR2 | 2.55E−09 | 0.391 | 0.412 | 0.155 | 2.04E−05 |
| IRF1 | 6.33E−09 | 0.341 | 0.347 | 0.108 | 5.08E−05 |
| APOL6 | 8.68E−09 | 0.505 | 0.653 | 0.352 | 6.97E−05 |
| GCA | 8.78E−09 | 0.430 | 0.372 | 0.122 | 7.05E−05 |
| COX4I1 | 1.26E−08 | 0.385 | 0.804 | 0.587 | 1.01E−04 |
| RNASE6 | 1.32E−08 | 0.369 | 0.648 | 0.432 | 1.06E−04 |
| SAMD9L | 1.58E−08 | 0.289 | 0.724 | 0.479 | 1.27E−04 |
| CLEC2B | 1.89E−08 | 0.517 | 0.533 | 0.249 | 1.52E−04 |
| FYB | 1.94E−08 | 0.515 | 0.764 | 0.507 | 1.55E−04 |
| DYNLT1 | 1.94E−08 | 0.412 | 0.377 | 0.169 | 1.56E−04 |
| RGS18 | 3.00E−08 | 0.332 | 0.513 | 0.249 | 2.41E−04 |
| COX5B | 3.68E−08 | 0.375 | 0.628 | 0.362 | 2.95E−04 |
| SP110 | 5.81E−08 | 0.595 | 0.663 | 0.394 | 4.66E−04 |
| GIMAP7 | 7.89E−08 | 0.489 | 0.528 | 0.254 | 6.33E−04 |
| PSME1 | 8.72E−08 | 0.361 | 0.779 | 0.512 | 7.00E−04 |
| MARCH1 | 1.41E−07 | 0.368 | 0.623 | 0.376 | 1.13E−03 |
| SAMD9 | 1.48E−07 | 0.302 | 0.668 | 0.423 | 1.19E−03 |
| NDUFA13 | 1.66E−07 | 0.548 | 0.548 | 0.3 | 1.33E−03 |
| PSMB8 | 1.94E−07 | 0.360 | 0.503 | 0.254 | 1.56E−03 |
| CARD16 | 2.03E−07 | 0.549 | 0.497 | 0.239 | 1.63E−03 |
| FCER1G | 2.33E−07 | 0.512 | 0.714 | 0.535 | 1.87E−03 |
| SERPING1 | 2.92E−07 | 0.328 | 0.352 | 0.16 | 2.34E−03 |
| SF3B14 | 3.16E−07 | 0.403 | 0.362 | 0.155 | 2.53E−03 |
| NDUFA1 | 3.45E−07 | 0.308 | 0.704 | 0.493 | 2.77E−03 |
| DDX60L | 5.03E−07 | 0.373 | 0.367 | 0.141 | 4.03E−03 |
| GSTK1 | 5.07E−07 | 0.524 | 0.442 | 0.221 | 4.06E−03 |
| LY6E | 5.91E−07 | 0.529 | 0.533 | 0.366 | 4.74E−03 |
| AP2S1 | 8.48E−07 | 0.344 | 0.397 | 0.183 | 6.80E−03 |
| UBB | 9.05E−07 | 0.513 | 0.739 | 0.512 | 7.26E−03 |
| EPSTI1 | 9.76E−07 | 0.252 | 0.734 | 0.493 | 7.83E−03 |
| MYD88 | 1.06E−06 | 0.355 | 0.317 | 0.122 | 8.51E−03 |
| GBP3 | 1.12E−06 | 0.459 | 0.291 | 0.089 | 8.97E−03 |
| LACTB | 1.13E−06 | 0.328 | 0.432 | 0.202 | 9.10E−03 |
| MRPS18C | 1.18E−06 | 0.258 | 0.417 | 0.192 | 9.51E−03 |
| TYMP | 1.23E−06 | 0.330 | 0.658 | 0.446 | 9.86E−03 |
| ITM2B | 1.32E−06 | 0.461 | 0.794 | 0.624 | 1.06E−02 |
| TCEB2 | 1.36E−06 | 0.322 | 0.457 | 0.272 | 1.09E−02 |
| HMGN2 | 1.41E−06 | 0.388 | 0.437 | 0.254 | 1.13E−02 |
| LGALS3BP | 1.55E−06 | 0.465 | 0.281 | 0.131 | 1.24E−02 |
| OAS1 | 1.69E−06 | 0.510 | 0.573 | 0.362 | 1.36E−02 |
| ANAPC11 | 1.77E−06 | 0.359 | 0.447 | 0.225 | 1.42E−02 |
| AMICA1 | 2.23E−06 | 0.418 | 0.467 | 0.249 | 1.79E−02 |
| OAZ1 | 2.36E−06 | 0.363 | 0.839 | 0.667 | 1.89E−02 |
| HLA-DQA1 | 2.59E−06 | 0.265 | 0.633 | 0.385 | 2.08E−02 |
| BLOC1S1 | 2.75E−06 | 0.382 | 0.412 | 0.216 | 2.21E−02 |
| HLA-C | 3.10E−06 | 0.338 | 0.854 | 0.789 | 2.48E−02 |
| GNB2L1 | 3.24E−06 | 0.275 | 0.839 | 0.695 | 2.60E−02 |
| RARRES3 | 3.64E−06 | 0.421 | 0.221 | 0.075 | 2.92E−02 |
| SPOP | 4.48E−06 | 0.260 | 0.327 | 0.136 | 3.59E−02 |
| PSTPIP2 | 4.58E−06 | 0.410 | 0.276 | 0.085 | 3.67E−02 |
| RPL18A | 4.82E−06 | 0.514 | 0.261 | 0.122 | 3.87E−02 |
| PSMB9 | 5.59E−06 | 0.278 | 0.462 | 0.282 | 4.48E−02 |

Differential expression results between responder and non-responder macrophage populations. Applicants observe that there is an up regulation not only of interferon sensing machinery in responders (expected because Applicants know interferon is in the microenvironment of these patients), but that there is some evidence of TNF signaling. This is important, as TNFa signaling has been reported to assist in tumor cell killing by infiltrating CTLs. The results may shed light on the source of TNF in the system and this could have broad impacts for understanding the mechanism of tumor cell killing in humans after administration of this drug in responding patients.

Table 8-10. BM vs. LMD comparisons. Differential expression between BM (brain metastasis patient) and LMD (“up” is BM, “down” is LMD).

Comparison of cells in CSF from patients with brain metastases to patients with LMD. Although not strictly a comparison of brain metastases to CSF, it could be a proxy for looking at phenotypes between LMD and disease strictly present in the brain and has not diagnostically spread to the meninges.

TABLE 8
CSF.BMvLMD.tumor.bimod.markers

| | | | | |
|---|---|---|---|---|
| p_val | avg_logFC | pct.1 | pct.2 | p_val_adj |

CSF.BMvLMD.tumor.bimod.markers.dn.1

| | | | | | |
|---|---|---|---|---|---|
| ANKRD30A | 0.00E+00 | −0.435 | 0.932 | 0.193 | 0.00E+00 |
| PABPC1 | 0.00E+00 | −1.710 | 0.983 | 0.978 | 0.00E+00 |
| RPL19 | 0.00E+00 | −2.698 | 0.998 | 0.835 | 0.00E+00 |
| EIF3H | 0.00E+00 | −3.178 | 0.675 | 0.709 | 0.00E+00 |
| RPL12 | 1.48E−323 | −2.990 | 0.966 | 0.862 | 1.69E−319 |
| MTRNR2L2 | 7.45E−298 | −3.622 | 0.282 | 0.63 | 8.49E−294 |
| RPS28 | 4.32E−273 | −1.112 | 0.875 | 0.719 | 4.93E−269 |
| MIEN1 | 4.83E−272 | −1.850 | 0.477 | 0.325 | 5.51E−268 |
| MTRNR2L8 | 9.10E−254 | −3.354 | 0.295 | 0.613 | 1.04E−249 |
| LDHA | 1.92E−253 | −1.637 | 0.699 | 0.58 | 2.19E−249 |
| RPL28 | 6.54E−247 | −0.951 | 0.909 | 0.741 | 7.45E−243 |
| MTRNR2L12 | 3.67E−241 | −3.327 | 0.163 | 0.579 | 4.18E−237 |
| RPS25 | 1.99E−230 | −0.982 | 0.864 | 0.687 | 2.26E−226 |
| RPL13A | 3.56E−219 | −1.126 | 0.946 | 0.844 | 4.06E−215 |
| SNHG5 | 3.70E−215 | −0.603 | 0.935 | 0.555 | 4.22E−211 |
| RPSA | 6.10E−213 | −1.387 | 0.379 | 0.522 | 6.95E−209 |
| XBP1 | 7.78E−213 | −0.982 | 0.747 | 0.468 | 8.87E−209 |
| MTRNR2L3 | 2.39E−208 | −1.855 | 0.575 | 0.573 | 2.73E−204 |
| EEF1D | 5.62E−208 | −0.856 | 0.776 | 0.567 | 6.40E−204 |
| RPL10 | 5.39E−204 | −0.931 | 0.695 | 0.521 | 6.15E−200 |
| SNHG6 | 3.65E−203 | −1.199 | 0.593 | 0.433 | 4.16E−199 |
| YBX1 | 1.68E−202 | −1.398 | 0.59 | 0.56 | 1.92E−198 |
| AZGP1 | 8.84E−199 | −2.991 | 0.004 | 0.496 | 1.01E−194 |
| TMSB10 | 1.08E−198 | −1.414 | 0.968 | 0.896 | 1.23E−194 |
| RPS7 | 7.04E−198 | −0.538 | 0.856 | 0.57 | 8.03E−194 |
| RPS9 | 3.24E−197 | −0.668 | 0.911 | 0.694 | 3.69E−193 |
| SNHG8 | 4.50E−197 | −1.182 | 0.585 | 0.483 | 5.13E−193 |
| NCL | 7.35E−195 | −1.021 | 0.866 | 0.758 | 8.38E−191 |
| UQCRB | 3.54E−194 | −0.703 | 0.804 | 0.549 | 4.03E−190 |
| ARPC5 | 1.68E−191 | −0.606 | 0.832 | 0.449 | 1.92E−187 |
| RPS3 | 6.57E−188 | −1.027 | 0.924 | 0.813 | 7.49E−184 |
| RNA28S5 | 2.19E−183 | −1.229 | 0.948 | 0.845 | 2.50E−179 |
| ENO1 | 3.21E−183 | −1.115 | 0.701 | 0.626 | 3.65E−179 |
| SSR4 | 3.59E−182 | −0.657 | 0.84 | 0.508 | 4.09E−178 |
| MYL12B | 1.61E−181 | −0.474 | 0.91 | 0.581 | 1.83E−177 |
| SYNGR2 | 2.59E−180 | −0.378 | 0.808 | 0.436 | 2.95E−176 |
| UTP23 | 5.90E−180 | −2.559 | 0.276 | 0.469 | 6.72E−176 |
| RPS24 | 6.81E−180 | −1.015 | 0.997 | 0.925 | 7.76E−176 |
| RPL4 | 1.61E−179 | −0.745 | 0.888 | 0.723 | 1.84E−175 |
| CD44 | 1.34E−177 | −3.356 | 0.163 | 0.478 | 1.53E−173 |
| GADD45GIP1 | 4.17E−175 | −0.678 | 0.659 | 0.436 | 4.75E−171 |
| RPL36 | 5.89E−175 | −0.712 | 0.915 | 0.763 | 6.71E−171 |
| ERBB2 | 1.61E−174 | −1.553 | 0.34 | 0.278 | 1.83E−170 |
| RPL14 | 8.87E−173 | −0.776 | 0.921 | 0.762 | 1.01E−168 |
| GAS5 | 4.15E−171 | −1.051 | 0.951 | 0.719 | 4.73E−167 |
| DSP | 5.51E−171 | −0.852 | 0.642 | 0.439 | 6.28E−167 |
| RPL3 | 7.51E−171 | −0.539 | 0.893 | 0.627 | 8.56E−167 |
| CD59 | 6.43E−170 | −1.158 | 0.534 | 0.402 | 7.33E−166 |
| CYCS | 2.36E−165 | −0.532 | 0.728 | 0.44 | 2.69E−161 |
| NBEAL1 | 6.07E−165 | −2.360 | 0.276 | 0.404 | 6.92E−161 |
| ILF2 | 3.11E−164 | −0.389 | 0.643 | 0.339 | 3.55E−160 |
| RPL23A | 1.47E−163 | −0.566 | 0.893 | 0.654 | 1.68E−159 |
| RPL22 | 1.99E−162 | −1.048 | 0.485 | 0.497 | 2.26E−158 |
| RPL9 | 2.87E−161 | −0.641 | 0.822 | 0.578 | 3.27E−157 |
| YWHAZ | 5.91E−161 | −0.944 | 0.804 | 0.651 | 6.73E−157 |
| RPS10 | 6.10E−160 | −0.404 | 0.828 | 0.544 | 6.95E−156 |
| RPS5 | 8.03E−160 | −0.800 | 0.979 | 0.86 | 9.16E−156 |
| NDUFS6 | 1.00E−159 | −0.864 | 0.616 | 0.424 | 1.14E−155 |
| NOL7 | 1.45E−159 | −0.786 | 0.632 | 0.468 | 1.66E−155 |
| MALAT1 | 2.60E−159 | −2.243 | 0.986 | 0.957 | 2.97E−155 |
| C17orf76-AS1 | 2.21E−158 | −0.685 | 0.777 | 0.565 | 2.52E−154 |
| RAD21 | 3.30E−158 | −1.207 | 0.545 | 0.541 | 3.76E−154 |
| RPS3A | 3.70E−158 | −0.518 | 0.707 | 0.466 | 4.21E−154 |
| EEF1A1 | 1.15E−157 | −0.685 | 0.94 | 0.751 | 1.31E−153 |
| SYNCRIP | 3.13E−157 | −0.708 | 0.565 | 0.378 | 3.57E−153 |
| EIF3E | 4.61E−154 | −1.041 | 0.524 | 0.535 | 5.26E−150 |
| RPL6 | 1.68E−153 | −0.517 | 0.805 | 0.551 | 1.91E−149 |
| DNAJB1 | 1.80E−153 | −0.365 | 0.723 | 0.398 | 2.05E−149 |
| ENAH | 3.76E−152 | −0.319 | 0.583 | 0.244 | 4.29E−148 |
| TM9SF2 | 4.27E−152 | −0.677 | 0.697 | 0.465 | 4.86E−148 |
| PRDX2 | 2.72E−150 | −0.468 | 0.644 | 0.398 | 3.10E−146 |
| HSPD1 | 6.59E−150 | −0.720 | 0.842 | 0.683 | 7.51E−146 |
| RPL13 | 2.65E−148 | −0.930 | 0.983 | 0.938 | 3.02E−144 |
| APLP2 | 2.68E−148 | −0.542 | 0.713 | 0.459 | 3.05E−144 |
| CCT5 | 5.51E−148 | −0.889 | 0.611 | 0.483 | 6.29E−144 |
| MYEOV2 | 7.82E−148 | −0.320 | 0.759 | 0.41 | 8.92E−144 |
| ENY2 | 1.27E−147 | −0.864 | 0.579 | 0.462 | 1.44E−143 |
| VIMP | 2.15E−147 | −0.326 | 0.629 | 0.302 | 2.46E−143 |
| TMEM123 | 2.75E−147 | −0.851 | 0.62 | 0.476 | 3.13E−143 |
| TMEM70 | 5.49E−147 | −0.961 | 0.383 | 0.285 | 6.26E−143 |
| EEF1B2 | 2.01E−146 | −0.455 | 0.844 | 0.59 | 2.29E−142 |
| RPS2 | 4.94E−146 | −0.309 | 0.96 | 0.656 | 5.63E−142 |
| HDLBP | 2.98E−145 | −0.304 | 0.783 | 0.457 | 3.40E−141 |
| C6orf48 | 3.98E−144 | −0.925 | 0.491 | 0.391 | 4.54E−140 |
| NDUFB9 | 8.51E−144 | −0.481 | 0.806 | 0.567 | 9.70E−140 |
| SLC38A2 | 1.39E−143 | −0.988 | 0.518 | 0.385 | 1.58E−139 |
| RPL8 | 2.26E−143 | −0.734 | 0.985 | 0.872 | 2.57E−139 |
| CSTB | 1.07E−142 | −0.543 | 0.722 | 0.426 | 1.22E−138 |
| GDI2 | 3.36E−142 | −0.573 | 0.636 | 0.438 | 3.83E−138 |
| LGALS1 | 3.68E−142 | −2.351 | 0.094 | 0.47 | 4.20E−138 |
| VMP1 | 1.14E−141 | −0.869 | 0.484 | 0.39 | 1.30E−137 |
| IQGAP1 | 3.76E−141 | −0.456 | 0.82 | 0.53 | 4.29E−137 |
| PSMA7 | 1.00E−140 | −0.439 | 0.864 | 0.595 | 1.14E−136 |
| TPI1 | 1.69E−140 | −0.391 | 0.824 | 0.54 | 1.92E−136 |
| CNIH4 | 2.85E−140 | −0.491 | 0.619 | 0.345 | 3.24E−136 |
| ZFAS1 | 3.87E−140 | −0.336 | 0.913 | 0.602 | 4.41E−136 |
| TMEM14C | 1.64E−139 | −0.613 | 0.58 | 0.402 | 1.87E−135 |
| ACTN4 | 3.32E−138 | −0.655 | 0.551 | 0.357 | 3.78E−134 |
| PAPOLA | 9.45E−138 | −0.387 | 0.712 | 0.422 | 1.08E−133 |
| SEPP1 | 1.61E−137 | −0.361 | 0.672 | 0.294 | 1.83E−133 |
| HSBP1 | 1.89E−137 | −0.998 | 0.534 | 0.464 | 2.16E−133 |
| PPP1CB | 2.54E−137 | −0.814 | 0.567 | 0.414 | 2.90E−133 |
| MTDH | 2.59E−137 | −0.836 | 0.562 | 0.459 | 2.95E−133 |
| MIF | 3.59E−137 | −1.438 | 0.285 | 0.395 | 4.10E−133 |
| APP | 3.99E−137 | −0.864 | 0.593 | 0.456 | 4.55E−133 |
| COX5B | 5.09E−137 | −0.359 | 0.78 | 0.473 | 5.80E−133 |
| ANAPC11 | 5.42E−137 | −0.317 | 0.769 | 0.451 | 6.18E−133 |
| RPS21 | 6.21E−136 | −0.728 | 0.999 | 0.937 | 7.08E−132 |
| C8orf59 | 2.79E−135 | −0.319 | 0.729 | 0.434 | 3.18E−131 |
| CD47 | 2.82E−135 | −1.209 | 0.343 | 0.281 | 3.22E−131 |
| VDAC2 | 4.09E−134 | −0.789 | 0.434 | 0.336 | 4.66E−130 |
| SUB1 | 6.03E−134 | −0.289 | 0.866 | 0.534 | 6.87E−130 |
| SCGB2A2 | 2.16E−133 | −3.605 | 0.015 | 0.394 | 2.46E−129 |
| HNRNPU | 2.76E−133 | −0.694 | 0.717 | 0.551 | 3.14E−129 |
| SERBP1 | 4.04E−133 | −0.473 | 0.755 | 0.491 | 4.61E−129 |
| AMD1 | 6.57E−133 | −0.295 | 0.601 | 0.297 | 7.48E−129 |
| PDCD5 | 1.05E−132 | −0.596 | 0.505 | 0.319 | 1.19E−128 |
| TMA7 | 7.10E−132 | −0.609 | 0.607 | 0.44 | 8.09E−128 |
| HEBP2 | 1.23E−131 | −0.357 | 0.641 | 0.341 | 1.40E−127 |
| TRPS1 | 4.11E−131 | −1.221 | 0.492 | 0.511 | 4.68E−127 |
| RPL29 | 1.20E−130 | −0.783 | 0.518 | 0.437 | 1.37E−126 |
| PSAP | 1.34E−130 | −0.419 | 0.75 | 0.482 | 1.53E−126 |
| MT-ND4L | 1.51E−130 | −0.917 | 0.755 | 0.551 | 1.73E−126 |
| PSMB5 | 5.58E−130 | −0.357 | 0.531 | 0.278 | 6.36E−126 |
| PRKDC | 5.84E−130 | −1.230 | 0.397 | 0.414 | 6.66E−126 |
| RPS16 | 1.13E−129 | −0.694 | 0.995 | 0.881 | 1.29E−125 |
| RBM3 | 2.06E−129 | −0.399 | 0.669 | 0.381 | 2.34E−125 |
| DDX17 | 5.34E−129 | −0.586 | 0.654 | 0.451 | 6.09E−125 |
| HLA-C | 7.10E−129 | −0.312 | 0.728 | 0.394 | 8.10E−125 |
| SET | 2.78E−128 | −0.665 | 0.706 | 0.517 | 3.17E−124 |
| OCIAD2 | 2.80E−128 | −0.278 | 0.563 | 0.273 | 3.20E−124 |
| NDUFA6 | 3.71E−128 | −0.386 | 0.712 | 0.408 | 4.23E−124 |
| SSB | 4.40E−128 | −0.451 | 0.594 | 0.357 | 5.02E−124 |
| HSD17B12 | 7.06E−128 | −0.519 | 0.564 | 0.331 | 8.04E−124 |
| FTH1 | 1.18E−127 | −0.749 | 0.932 | 0.805 | 1.35E−123 |
| CLTC | 2.54E−127 | −0.394 | 0.653 | 0.377 | 2.90E−123 |
| ZFP36L1 | 3.03E−127 | −0.616 | 0.75 | 0.476 | 3.46E−123 |
| PDCD10 | 9.79E−127 | −0.517 | 0.606 | 0.372 | 1.12E−122 |
| PPIA | 2.24E−126 | −0.300 | 0.665 | 0.379 | 2.55E−122 |
| TMEM261 | 5.82E−126 | −0.505 | 0.504 | 0.28 | 6.64E−122 |
| CBX3 | 2.80E−125 | −0.597 | 0.815 | 0.603 | 3.19E−121 |
| LSM7 | 1.19E−124 | −0.297 | 0.636 | 0.349 | 1.36E−120 |
| GTF3C6 | 9.51E−124 | −0.306 | 0.453 | 0.202 | 1.08E−119 |
| DSC2 | 3.61E−123 | −0.569 | 0.335 | 0.161 | 4.12E−119 |
| HDAC2 | 7.81E−123 | −0.599 | 0.501 | 0.319 | 8.91E−119 |
| SFPQ | 9.88E−123 | −0.347 | 0.697 | 0.409 | 1.13E−118 |
| EIF2S2 | 1.09E−122 | −0.827 | 0.531 | 0.453 | 1.25E−118 |
| SQLE | 2.61E−122 | −1.368 | 0.299 | 0.405 | 2.98E−118 |
| NPM1 | 4.41E−122 | −0.279 | 0.86 | 0.584 | 5.03E−118 |
| DNM1L | 1.31E−121 | −0.337 | 0.438 | 0.195 | 1.49E−117 |
| ARGLU1 | 5.16E−121 | −0.604 | 0.566 | 0.379 | 5.89E−117 |
| NHP2 | 5.56E−121 | −0.302 | 0.626 | 0.345 | 6.34E−117 |
| TPR | 1.84E−120 | −0.712 | 0.605 | 0.427 | 2.10E−116 |
| WDR83OS | 1.92E−120 | −0.272 | 0.613 | 0.351 | 2.19E−116 |
| SNRPD3 | 4.75E−120 | −0.791 | 0.408 | 0.363 | 5.41E−116 |
| MTRNR2L1 | 6.64E−120 | −1.118 | 0.981 | 0.929 | 7.56E−116 |
| KTN1 | 8.24E−120 | −0.606 | 0.747 | 0.529 | 9.39E−116 |
| POLR2K | 9.95E−120 | −0.297 | 0.589 | 0.31 | 1.13E−115 |
| PDIA6 | 5.95E−119 | −0.342 | 0.742 | 0.458 | 6.78E−115 |
| PET100 | 8.07E−119 | −0.304 | 0.593 | 0.32 | 9.19E−115 |
| DYNC1H1 | 8.91E−119 | −0.566 | 0.487 | 0.306 | 1.02E−114 |
| ANP32B | 1.09E−118 | −0.635 | 0.614 | 0.444 | 1.25E−114 |
| UBA52 | 1.73E−118 | −0.428 | 0.948 | 0.732 | 1.97E−114 |
| H2AFJ | 5.84E−118 | −0.278 | 0.6 | 0.326 | 6.65E−114 |
| THBS1 | 1.21E−117 | −1.850 | 0.302 | 0.259 | 1.38E−113 |
| RPS27 | 5.35E−117 | −0.282 | 0.923 | 0.65 | 6.10E−113 |
| PRPF40A | 5.75E−117 | −0.326 | 0.66 | 0.375 | 6.56E−113 |
| TAGLN2 | 1.51E−116 | −0.384 | 0.71 | 0.425 | 1.73E−112 |
| MGST3 | 1.79E−116 | −0.316 | 0.583 | 0.305 | 2.05E−112 |
| GPX1 | 3.41E−116 | −0.546 | 0.496 | 0.323 | 3.89E−112 |
| NUPR1 | 3.74E−116 | −1.134 | 0.36 | 0.329 | 4.26E−112 |
| HSPA1B | 5.41E−116 | −0.344 | 0.543 | 0.266 | 6.17E−112 |
| CDC37 | 6.93E−116 | −0.265 | 0.418 | 0.189 | 7.90E−112 |
| MRPL13 | 8.65E−116 | −0.718 | 0.46 | 0.352 | 9.86E−112 |
| CYBA | 1.67E−115 | −0.288 | 0.303 | 0.096 | 1.90E−111 |
| CCDC47 | 1.87E−115 | −0.493 | 0.587 | 0.387 | 2.13E−111 |
| RAD23A | 3.19E−115 | −0.465 | 0.558 | 0.373 | 3.63E−111 |
| DBI | 6.10E−115 | −0.612 | 0.832 | 0.625 | 6.96E−111 |
| ATP5A1 | 6.22E−115 | −0.261 | 0.597 | 0.332 | 7.09E−111 |
| EIF5B | 1.73E−114 | −0.927 | 0.496 | 0.419 | 1.98E−110 |
| EIF3L | 2.38E−114 | −0.826 | 0.392 | 0.375 | 2.72E−110 |
| MT-ATP8 | 4.70E−114 | −0.848 | 0.695 | 0.505 | 5.35E−110 |
| SF3B5 | 4.99E−114 | −0.625 | 0.44 | 0.303 | 5.68E−110 |
| YWHAQ | 7.39E−114 | −0.587 | 0.46 | 0.322 | 8.42E−110 |
| HNRNPR | 8.87E−114 | −0.390 | 0.606 | 0.37 | 1.01E−109 |
| DEK | 1.04E−113 | −1.314 | 0.45 | 0.524 | 1.18E−109 |
| GNAS | 1.19E−113 | −0.395 | 0.72 | 0.466 | 1.36E−109 |
| TTC3 | 4.28E−113 | −0.339 | 0.755 | 0.456 | 4.88E−109 |
| SYNE2 | 7.15E−113 | −1.491 | 0.336 | 0.477 | 8.15E−109 |
| C11orf31 | 7.58E−113 | −0.284 | 0.542 | 0.29 | 8.64E−109 |
| SUMO2 | 2.70E−112 | −0.323 | 0.578 | 0.338 | 3.08E−108 |
| PDCD6 | 5.98E−112 | −0.574 | 0.452 | 0.29 | 6.82E−108 |
| NFIB | 6.15E−112 | −1.678 | 0.087 | 0.383 | 7.01E−108 |
| ATP5C1 | 9.68E−112 | −0.326 | 0.715 | 0.447 | 1.10E−107 |
| GOLGB1 | 1.41E−111 | −0.856 | 0.523 | 0.376 | 1.61E−107 |
| FBL | 2.23E−111 | −0.853 | 0.383 | 0.374 | 2.54E−107 |
| ST13 | 2.27E−111 | −0.688 | 0.483 | 0.394 | 2.59E−107 |
| PAICS | 3.01E−111 | −0.946 | 0.359 | 0.344 | 3.43E−107 |
| MTRNR2L11 | 3.17E−111 | −1.490 | 0.179 | 0.301 | 3.62E−107 |
| HNRNPH1 | 4.01E−111 | −0.393 | 0.638 | 0.383 | 4.57E−107 |
| RPL32 | 4.86E−111 | −0.373 | 1 | 0.918 | 5.54E−107 |
| EIF5A | 5.12E−111 | −0.381 | 0.496 | 0.285 | 5.84E−107 |
| IDH2 | 6.23E−111 | −0.506 | 0.497 | 0.281 | 7.10E−107 |
| PEG10 | 1.34E−110 | −2.080 | 0.001 | 0.302 | 1.53E−106 |
| FAU | 1.85E−110 | −0.348 | 0.955 | 0.741 | 2.10E−106 |
| LUC7L3 | 2.35E−110 | −0.473 | 0.622 | 0.377 | 2.68E−106 |
| NOP58 | 2.92E−110 | −0.565 | 0.57 | 0.396 | 3.33E−106 |
| MTRNR2L6 | 3.62E−110 | −1.903 | 0.092 | 0.334 | 4.13E−106 |
| ATF4 | 4.47E−110 | −0.307 | 0.661 | 0.404 | 5.10E−106 |
| RNA18S5 | 4.53E−110 | −2.779 | 0 | 0.296 | 5.17E−106 |
| TPM1 | 6.62E−110 | −1.176 | 0.486 | 0.537 | 7.55E−106 |
| ATP5EP2 | 1.02E−109 | −0.698 | 0.411 | 0.338 | 1.16E−105 |
| RBM39 | 1.10E−109 | −0.480 | 0.665 | 0.44 | 1.25E−105 |
| GOLGA4 | 2.26E−109 | −0.889 | 0.509 | 0.452 | 2.57E−105 |
| XRCC6 | 2.36E−109 | −0.682 | 0.482 | 0.378 | 2.69E−105 |
| PARP1 | 3.74E−109 | −0.277 | 0.58 | 0.311 | 4.26E−105 |
| MT-CO2 | 5.20E−109 | −0.616 | 0.982 | 0.772 | 5.92E−105 |
| HIST1H4C | 1.09E−107 | −2.231 | 0.307 | 0.546 | 1.25E−103 |
| FAM213A | 1.15E−107 | −0.618 | 0.468 | 0.338 | 1.31E−103 |
| RBX1 | 1.62E−107 | −0.407 | 0.548 | 0.331 | 1.85E−103 |
| ILF3 | 1.75E−107 | −0.599 | 0.536 | 0.367 | 2.00E−103 |
| STRA13 | 2.03E−107 | −0.315 | 0.509 | 0.279 | 2.31E−103 |
| TXN | 2.13E−107 | −0.277 | 0.88 | 0.594 | 2.42E−103 |
| CDK4 | 4.31E−107 | −0.786 | 0.337 | 0.254 | 4.91E−103 |
| MGP | 8.28E−107 | −3.957 | 0.065 | 0.372 | 9.44E−103 |
| UGP2 | 1.68E−106 | −0.489 | 0.473 | 0.293 | 1.92E−102 |
| C19orf43 | 2.50E−106 | −0.648 | 0.419 | 0.341 | 2.84E−102 |
| HNRNPD | 2.64E−106 | −0.550 | 0.503 | 0.333 | 3.01E−102 |
| U2SURP | 2.74E−106 | −0.295 | 0.612 | 0.345 | 3.13E−102 |
| MT-TV | 3.00E−106 | −0.461 | 0.581 | 0.308 | 3.42E−102 |
| RPL37 | 4.33E−106 | −0.320 | 0.994 | 0.813 | 4.94E−102 |
| RAD23B | 7.38E−106 | −0.272 | 0.498 | 0.265 | 8.41E−102 |
| DDX46 | 1.07E−105 | −0.292 | 0.511 | 0.266 | 1.22E−101 |
| MORF4L2 | 1.37E−105 | −0.270 | 0.64 | 0.358 | 1.57E−101 |
| RPF2 | 1.38E−105 | −0.587 | 0.386 | 0.254 | 1.57E−101 |
| HNRNPM | 1.86E−105 | −0.465 | 0.651 | 0.429 | 2.11E−101 |
| EIF3M | 4.20E−105 | −0.464 | 0.498 | 0.329 | 4.79E−101 |
| RPL18A | 5.29E−105 | −1.025 | 0.23 | 0.347 | 6.04E−101 |
| MAPKAP1 | 5.50E−105 | −1.572 | 0.25 | 0.313 | 6.27E−101 |
| RNF213 | 9.46E−105 | −0.276 | 0.561 | 0.271 | 1.08E−100 |
| NAP1L1 | 1.31E−104 | −0.304 | 0.778 | 0.508 | 1.49E−100 |
| DDX21 | 1.92E−104 | −0.559 | 0.586 | 0.406 | 2.19E−100 |
| CHD2 | 4.37E−104 | −0.354 | 0.416 | 0.203 | 4.98E−100 |
| IPO5 | 4.87E−104 | −1.051 | 0.241 | 0.303 | 5.55E−100 |
| HNRNPC | 7.66E−104 | −0.269 | 0.745 | 0.462 | 8.74E−100 |
| SEC63 | 1.28E−103 | −0.287 | 0.5 | 0.259 | 1.46E−99 |
| BCLAF1 | 1.63E−103 | −0.298 | 0.554 | 0.306 | 1.86E−99 |
| HNRNPA2B1 | 2.43E−103 | −0.397 | 0.931 | 0.709 | 2.77E−99 |
| RARRES1 | 2.45E−103 | −2.484 | 0.009 | 0.279 | 2.79E−99 |
| MMADHC | 3.99E−103 | −0.264 | 0.395 | 0.183 | 4.54E−99 |
| MYC | 4.51E−103 | −1.662 | 0.011 | 0.314 | 5.14E−99 |
| DNAJB11 | 6.42E−103 | −0.518 | 0.429 | 0.283 | 7.32E−99 |
| VAPA | 1.15E−102 | −0.688 | 0.435 | 0.347 | 1.31E−98 |
| ACTR3 | 1.15E−102 | −0.304 | 0.543 | 0.301 | 1.31E−98 |
| TSFM | 2.74E−102 | −0.729 | 0.238 | 0.148 | 3.12E−98 |
| ZC3H15 | 3.62E−102 | −0.256 | 0.634 | 0.355 | 4.13E−98 |
| TIMM8B | 3.80E−102 | −0.973 | 0.278 | 0.282 | 4.34E−98 |
| HMGCS1 | 4.64E−102 | −0.871 | 0.372 | 0.3 | 5.29E−98 |
| SUCLG1 | 4.91E−102 | −0.441 | 0.48 | 0.282 | 5.60E−98 |
| SMEK1 | 7.75E−102 | −0.296 | 0.405 | 0.204 | 8.83E−98 |
| SSR1 | 7.77E−102 | −0.407 | 0.473 | 0.276 | 8.85E−98 |
| TRA2B | 1.11E−101 | −0.315 | 0.554 | 0.312 | 1.26E−97 |
| NDUFB5 | 1.31E−101 | −0.419 | 0.423 | 0.246 | 1.50E−97 |
| PHB | 1.10E−100 | −0.381 | 0.425 | 0.246 | 1.25E−96 |
| TIMM13 | 1.38E−100 | −0.257 | 0.523 | 0.288 | 1.57E−96 |
| CAPZB | 1.47E−100 | −0.409 | 0.447 | 0.262 | 1.68E−96 |
| DPM2 | 1.65E−100 | −0.881 | 0.287 | 0.244 | 1.88E−96 |
| CHCHD1 | 1.80E−100 | −0.379 | 0.466 | 0.283 | 2.05E−96 |
| GNL3 | 1.95E−100 | −0.445 | 0.507 | 0.317 | 2.22E−96 |
| GNB1 | 2.71E−100 | −0.293 | 0.5 | 0.277 | 3.09E−96 |
| RPL5 | 2.84E−100 | −0.398 | 0.991 | 0.818 | 3.23E−96 |
| ASPH | 2.85E−100 | −1.277 | 0.018 | 0.333 | 3.25E−96 |
| HMGB1 | 5.50E−100 | −0.713 | 0.69 | 0.526 | 6.27E−96 |
| ATRX | 9.44E−100 | −0.561 | 0.45 | 0.288 | 1.08E−95 |
| CTC-338M12.5 | 3.13E−99 | −2.451 | 0.119 | 0.276 | 3.57E−95 |
| RPL26 | 4.19E−99 | −0.267 | 0.994 | 0.78 | 4.77E−95 |
| IFITM1 | 5.19E−99 | −0.534 | 0.216 | 0.1 | 5.92E−95 |
| TUBB | 7.33E−99 | −0.902 | 0.574 | 0.51 | 8.35E−95 |
| HLA-B | 7.44E−99 | −0.336 | 0.87 | 0.588 | 8.49E−95 |
| APOD | 1.13E−98 | −1.462 | 0.142 | 0.216 | 1.29E−94 |
| ACTN1 | 1.54E−98 | −0.584 | 0.431 | 0.266 | 1.75E−94 |
| MRPL51 | 1.54E−98 | −0.284 | 0.548 | 0.316 | 1.75E−94 |
| EBNA1BP2 | 2.51E−98 | −0.370 | 0.408 | 0.227 | 2.86E−94 |
| RTN4 | 3.92E−98 | −0.440 | 0.55 | 0.366 | 4.47E−94 |
| MT-ND5 | 3.94E−98 | −0.367 | 0.875 | 0.598 | 4.49E−94 |
| MT-ND1 | 5.48E−98 | −0.712 | 0.865 | 0.688 | 6.25E−94 |
| NOP56 | 6.24E−98 | −0.610 | 0.462 | 0.34 | 7.12E−94 |
| PNISR | 6.78E−98 | −0.269 | 0.455 | 0.216 | 7.72E−94 |
| RBM17 | 1.07E−97 | −0.667 | 0.404 | 0.335 | 1.21E−93 |
| CCDC6 | 2.12E−97 | −0.254 | 0.527 | 0.303 | 2.42E−93 |
| MED1 | 2.17E−97 | −1.269 | 0.161 | 0.269 | 2.47E−93 |
| NME1 | 2.78E−97 | −0.345 | 0.566 | 0.351 | 3.17E−93 |
| CTNNB1 | 3.02E−97 | −1.073 | 0.337 | 0.363 | 3.44E−93 |
| RBM25 | 4.27E−97 | −0.755 | 0.521 | 0.412 | 4.86E−93 |
| HES1 | 5.15E−97 | −0.358 | 0.742 | 0.456 | 5.87E−93 |
| IARS2 | 5.84E−97 | −0.271 | 0.473 | 0.251 | 6.65E−93 |
| UBE2V2 | 5.96E−97 | −0.513 | 0.472 | 0.318 | 6.79E−93 |
| VPS26A | 1.11E−96 | −0.565 | 0.432 | 0.316 | 1.27E−92 |
| TPD52 | 2.16E−96 | −0.294 | 0.57 | 0.335 | 2.46E−92 |
| AZIN1 | 2.38E−96 | −0.861 | 0.321 | 0.306 | 2.72E−92 |
| LSM4 | 2.90E−96 | −0.378 | 0.446 | 0.27 | 3.31E−92 |
| RPS29 | 3.40E−96 | −0.446 | 1 | 0.953 | 3.88E−92 |
| JMJD1C | 5.34E−96 | −0.979 | 0.393 | 0.362 | 6.09E−92 |
| TTC17 | 6.21E−96 | −0.274 | 0.374 | 0.187 | 7.08E−92 |
| GGCT | 6.93E−96 | −0.287 | 0.672 | 0.417 | 7.90E−92 |
| PSMC3 | 7.12E−96 | −0.334 | 0.514 | 0.288 | 8.12E−92 |
| ITGB1 | 7.13E−96 | −0.800 | 0.355 | 0.31 | 8.13E−92 |
| SH3BGRL3 | 7.46E−96 | −0.261 | 0.406 | 0.196 | 8.50E−92 |
| C4orf3 | 9.11E−96 | −0.284 | 0.519 | 0.296 | 1.04E−91 |
| HAP1 | 1.02E−95 | −1.625 | 0.037 | 0.275 | 1.16E−91 |
| ESR1 | 1.12E−95 | −0.596 | 0.235 | 0.106 | 1.27E−91 |
| MRPL47 | 1.50E−95 | −0.294 | 0.427 | 0.224 | 1.71E−91 |
| NARS | 2.27E−95 | −0.546 | 0.418 | 0.29 | 2.59E−91 |
| PAPSS1 | 3.02E−95 | −0.284 | 0.309 | 0.146 | 3.44E−91 |
| HNRNPH3 | 3.66E−95 | −0.342 | 0.502 | 0.305 | 4.18E−91 |
| METTL12 | 4.41E−95 | −1.323 | 0.173 | 0.344 | 5.03E−91 |
| G3BP1 | 8.92E−95 | −0.377 | 0.491 | 0.305 | 1.02E−90 |
| PHB2 | 1.06E−94 | −0.300 | 0.459 | 0.263 | 1.21E−90 |
| RBMX | 1.40E−94 | −0.405 | 0.617 | 0.42 | 1.60E−90 |
| PHIP | 3.21E−94 | −0.386 | 0.464 | 0.265 | 3.66E−90 |
| BDP1 | 4.62E−94 | −0.422 | 0.482 | 0.272 | 5.26E−90 |
| LAMP2 | 5.82E−94 | −0.503 | 0.44 | 0.283 | 6.63E−90 |
| MT-ND2 | 8.12E−94 | −0.623 | 0.992 | 0.84 | 9.26E−90 |
| PRDX4 | 9.21E−94 | −0.502 | 0.406 | 0.267 | 1.05E−89 |
| RPL31 | 1.86E−93 | −0.678 | 0.996 | 0.92 | 2.12E−89 |
| ARPC5L | 2.29E−93 | −0.375 | 0.4 | 0.227 | 2.61E−89 |
| MRPS7 | 5.12E−93 | −0.347 | 0.387 | 0.221 | 5.83E−89 |
| LDHB | 1.07E−92 | −1.664 | 0.019 | 0.271 | 1.22E−88 |
| TSC22D1 | 2.00E−92 | −0.322 | 0.525 | 0.287 | 2.28E−88 |
| GBP2 | 3.42E−92 | −0.332 | 0.388 | 0.157 | 3.90E−88 |
| ZFP36L2 | 6.30E−92 | −0.975 | 0.351 | 0.388 | 7.18E−88 |
| POLR2B | 7.19E−92 | −0.506 | 0.377 | 0.244 | 8.20E−88 |
| TSPO | 7.94E−92 | −0.657 | 0.462 | 0.379 | 9.05E−88 |
| PLSCR1 | 8.13E−92 | −0.350 | 0.342 | 0.164 | 9.27E−88 |
| FXR1 | 9.12E−92 | −0.449 | 0.513 | 0.357 | 1.04E−87 |
| RPLP0 | 1.34E−91 | −0.434 | 0.989 | 0.843 | 1.53E−87 |
| PRDX5 | 1.49E−91 | −0.259 | 0.462 | 0.252 | 1.70E−87 |
| NONO | 2.19E−91 | −0.411 | 0.502 | 0.319 | 2.50E−87 |
| SCGB1D2 | 2.37E−91 | −2.324 | 0.006 | 0.278 | 2.70E−87 |
| CP | 3.08E−91 | −2.312 | 0.004 | 0.259 | 3.51E−87 |
| TMEM258 | 5.42E−91 | −0.412 | 0.532 | 0.361 | 6.18E−87 |
| PPM1G | 1.83E−90 | −0.267 | 0.543 | 0.297 | 2.09E−86 |
| WDR43 | 2.23E−90 | −0.549 | 0.439 | 0.322 | 2.54E−86 |
| UCHL5 | 3.03E−90 | −0.296 | 0.414 | 0.214 | 3.45E−86 |
| BPTF | 3.72E−90 | −0.746 | 0.411 | 0.329 | 4.24E−86 |
| PIK3R1 | 4.52E−90 | −0.355 | 0.324 | 0.146 | 5.15E−86 |
| IPO7 | 4.66E−90 | −0.341 | 0.499 | 0.31 | 5.31E−86 |
| LAMP1 | 1.24E−89 | −0.341 | 0.482 | 0.284 | 1.42E−85 |
| RPS19 | 1.56E−89 | −0.587 | 0.997 | 0.934 | 1.78E−85 |
| MT-CO1 | 1.60E−89 | −0.819 | 0.986 | 0.865 | 1.83E−85 |
| ERGIC3 | 2.22E−89 | −0.266 | 0.443 | 0.251 | 2.54E−85 |
| PRMT1 | 3.02E−89 | −0.453 | 0.352 | 0.243 | 3.44E−85 |
| RPS27A | 3.44E−89 | −0.399 | 0.987 | 0.843 | 3.92E−85 |
| AHNAK | 3.95E−89 | −1.066 | 0.221 | 0.348 | 4.50E−85 |
| PPT1 | 4.43E−89 | −0.430 | 0.455 | 0.303 | 5.05E−85 |
| RPS20 | 5.44E−89 | −0.296 | 0.981 | 0.796 | 6.20E−85 |
| MAGOH | 5.44E−89 | −0.420 | 0.345 | 0.209 | 6.20E−85 |
| KPNB1 | 7.13E−89 | −0.512 | 0.482 | 0.342 | 8.13E−85 |
| HADHA | 7.80E−89 | −0.394 | 0.484 | 0.302 | 8.89E−85 |
| YBX3 | 9.39E−89 | −1.322 | 0.006 | 0.264 | 1.07E−84 |
| LMAN1 | 1.78E−88 | −0.673 | 0.378 | 0.318 | 2.02E−84 |
| CCNC | 3.63E−88 | −0.298 | 0.326 | 0.164 | 4.14E−84 |
| MRPL36 | 4.71E−88 | −0.963 | 0.22 | 0.265 | 5.36E−84 |
| USO1 | 1.26E−87 | −0.298 | 0.474 | 0.278 | 1.44E−83 |
| HM13 | 1.57E−87 | −0.262 | 0.391 | 0.211 | 1.79E−83 |
| C6orf62 | 1.77E−87 | −0.401 | 0.532 | 0.338 | 2.02E−83 |
| PSMC6 | 2.91E−87 | −0.280 | 0.388 | 0.203 | 3.32E−83 |
| TTC19 | 2.96E−87 | −0.348 | 0.449 | 0.271 | 3.37E−83 |
| TKT | 3.09E−87 | −0.613 | 0.445 | 0.362 | 3.52E−83 |
| ZNF146 | 6.43E−87 | −0.716 | 0.35 | 0.313 | 7.33E−83 |
| MDM4 | 7.77E−87 | −0.292 | 0.27 | 0.114 | 8.86E−83 |
| PAK1IP1 | 1.23E−86 | −1.103 | 0.225 | 0.353 | 1.40E−82 |
| CHD4 | 1.23E−86 | −0.579 | 0.476 | 0.356 | 1.41E−82 |
| TRIP11 | 1.29E−86 | −0.263 | 0.474 | 0.245 | 1.47E−82 |
| SENP6 | 2.18E−86 | −0.513 | 0.449 | 0.294 | 2.49E−82 |
| KIAA0020 | 2.73E−86 | −1.022 | 0.267 | 0.319 | 3.11E−82 |
| SLC25A39 | 7.32E−86 | −0.267 | 0.353 | 0.188 | 8.34E−82 |
| TMEM14B | 2.63E−85 | −0.528 | 0.436 | 0.332 | 3.00E−81 |
| SNRPD1 | 3.23E−85 | −0.853 | 0.356 | 0.359 | 3.69E−81 |
| LARS | 3.80E−85 | −0.531 | 0.471 | 0.341 | 4.33E−81 |
| MFGE8 | 3.87E−85 | −2.707 | 0.001 | 0.24 | 4.42E−81 |
| CAPRIN1 | 4.30E−85 | −0.389 | 0.371 | 0.231 | 4.90E−81 |
| ARMCX3 | 4.51E−85 | −0.393 | 0.289 | 0.148 | 5.14E−81 |
| MT-ATP6 | 4.60E−85 | −0.495 | 0.96 | 0.744 | 5.24E−81 |
| TBL1XR1 | 4.76E−85 | −0.673 | 0.415 | 0.344 | 5.43E−81 |
| TFAP2A | 6.80E−85 | −0.505 | 0.319 | 0.218 | 7.75E−81 |
| SF3B2 | 7.89E−85 | −0.460 | 0.381 | 0.247 | 8.99E−81 |
| ANXA5 | 7.96E−85 | −0.346 | 0.559 | 0.353 | 9.07E−81 |
| PHF20L1 | 1.84E−84 | −0.527 | 0.368 | 0.255 | 2.10E−80 |
| DDX18 | 2.29E−84 | −0.718 | 0.334 | 0.276 | 2.61E−80 |
| LBR | 4.65E−84 | −0.773 | 0.282 | 0.216 | 5.30E−80 |
| PABPC4 | 5.69E−84 | −0.355 | 0.425 | 0.268 | 6.48E−80 |
| PHF3 | 9.76E−84 | −0.289 | 0.511 | 0.302 | 1.11E−79 |
| MRFAP1 | 1.09E−83 | −0.380 | 0.387 | 0.24 | 1.24E−79 |
| SFT2D2 | 3.20E−83 | −0.484 | 0.283 | 0.176 | 3.64E−79 |
| PSMD11 | 3.41E−83 | −0.337 | 0.317 | 0.177 | 3.89E−79 |
| JUND | 3.43E−83 | −0.629 | 0.384 | 0.295 | 3.91E−79 |
| ANKRD12 | 3.48E−83 | −0.647 | 0.438 | 0.328 | 3.97E−79 |
| CAMK2N1 | 4.27E−83 | −0.283 | 0.352 | 0.178 | 4.87E−79 |
| DEGS1 | 4.63E−83 | −0.325 | 0.379 | 0.205 | 5.28E−79 |
| HNRNPA3 | 5.01E−83 | −0.309 | 0.471 | 0.284 | 5.71E−79 |
| PSMC4 | 6.66E−83 | −0.400 | 0.33 | 0.208 | 7.59E−79 |
| P4HA1 | 7.71E−83 | −0.306 | 0.377 | 0.195 | 8.78E−79 |
| EIF2S1 | 1.37E−82 | −0.312 | 0.378 | 0.214 | 1.56E−78 |
| RPL39 | 1.46E−82 | −0.378 | 0.992 | 0.862 | 1.67E−78 |
| PDHX | 2.24E−82 | −0.994 | 0.202 | 0.209 | 2.55E−78 |
| PYURF | 3.28E−82 | −0.444 | 0.453 | 0.304 | 3.74E−78 |
| MARCKS | 3.38E−82 | −0.323 | 0.446 | 0.249 | 3.85E−78 |
| SLC39A7 | 7.66E−82 | −0.321 | 0.389 | 0.239 | 8.73E−78 |
| S100A1 | 9.52E−82 | −0.615 | 0.154 | 0.097 | 1.09E−77 |
| SLTM | 1.29E−81 | −0.268 | 0.485 | 0.281 | 1.47E−77 |
| ZFR | 1.49E−81 | −0.254 | 0.469 | 0.254 | 1.70E−77 |
| RTN3 | 1.84E−81 | −0.784 | 0.274 | 0.302 | 2.10E−77 |
| KPNA4 | 2.14E−81 | −0.454 | 0.344 | 0.218 | 2.44E−77 |
| USP22 | 2.74E−81 | −0.311 | 0.33 | 0.184 | 3.12E−77 |
| DDX6 | 2.77E−81 | −0.337 | 0.36 | 0.214 | 3.15E−77 |
| PRPF4B | 3.05E−81 | −0.329 | 0.442 | 0.269 | 3.48E−77 |
| RPL30 | 3.81E−81 | −0.524 | 1 | 0.967 | 4.34E−77 |
| CHMP3 | 6.35E−81 | −0.367 | 0.421 | 0.276 | 7.24E−77 |
| RABEP1 | 9.94E−81 | −0.372 | 0.439 | 0.253 | 1.13E−76 |
| NBPF10 | 1.37E−80 | −0.278 | 0.268 | 0.129 | 1.57E−76 |
| DCD | 2.84E−80 | −2.237 | 0.006 | 0.246 | 3.24E−76 |
| CDH1 | 3.27E−80 | −0.375 | 0.318 | 0.176 | 3.72E−76 |
| N4BP2L2 | 8.71E−80 | −0.285 | 0.481 | 0.278 | 9.92E−76 |
| ZRANB2 | 1.03E−79 | −0.294 | 0.47 | 0.265 | 1.17E−75 |
| MYH9 | 1.06E−79 | −0.662 | 0.476 | 0.376 | 1.21E−75 |
| GRN | 3.05E−79 | −0.337 | 0.242 | 0.113 | 3.48E−75 |
| ZNF706 | 4.39E−79 | −0.625 | 0.367 | 0.306 | 5.00E−75 |
| CWC15 | 6.00E−79 | −0.598 | 0.372 | 0.279 | 6.84E−75 |
| PSMD12 | 6.79E−79 | −0.424 | 0.385 | 0.242 | 7.74E−75 |
| LARP1 | 7.25E−79 | −0.279 | 0.369 | 0.206 | 8.27E−75 |
| SMEK2 | 1.03E−78 | −0.298 | 0.389 | 0.217 | 1.17E−74 |
| DICER1 | 1.26E−78 | −0.498 | 0.32 | 0.209 | 1.44E−74 |
| LMO4 | 2.15E−78 | −1.203 | 0.204 | 0.396 | 2.45E−74 |
| NHP2L1 | 2.63E−78 | −0.623 | 0.337 | 0.297 | 3.00E−74 |
| MRPS12 | 2.76E−78 | −0.312 | 0.333 | 0.203 | 3.14E−74 |
| ZNF638 | 3.34E−78 | −0.363 | 0.44 | 0.266 | 3.80E−74 |
| MRPL34 | 3.48E−78 | −0.423 | 0.342 | 0.224 | 3.96E−74 |
| APEX1 | 4.44E−78 | −0.385 | 0.439 | 0.281 | 5.06E−74 |
| RNU6-6P | 5.07E−78 | −0.525 | 0.368 | 0.21 | 5.77E−74 |
| PNN | 6.80E−78 | −0.541 | 0.402 | 0.29 | 7.75E−74 |
| HIPK2 | 8.45E−78 | −0.320 | 0.307 | 0.155 | 9.63E−74 |
| ZBTB41 | 9.21E−78 | −0.266 | 0.272 | 0.122 | 1.05E−73 |
| CHCHD3 | 9.46E−78 | −0.281 | 0.345 | 0.194 | 1.08E−73 |
| EIF4G1 | 9.54E−78 | −0.502 | 0.426 | 0.311 | 1.09E−73 |
| PRPF38B | 1.03E−77 | −0.282 | 0.405 | 0.216 | 1.18E−73 |
| ORMDL3 | 1.26E−77 | −0.367 | 0.321 | 0.207 | 1.44E−73 |
| PSMD7 | 1.58E−77 | −0.521 | 0.284 | 0.201 | 1.80E−73 |
| TGOLN2 | 1.92E−77 | −0.445 | 0.341 | 0.229 | 2.19E−73 |
| SLK | 2.13E−77 | −0.287 | 0.52 | 0.296 | 2.43E−73 |
| MED13 | 2.29E−77 | −0.418 | 0.327 | 0.195 | 2.61E−73 |
| SMC4 | 2.75E−77 | −0.877 | 0.377 | 0.33 | 3.13E−73 |
| 6-Mar | 2.88E−77 | −0.429 | 0.341 | 0.235 | 3.28E−73 |
| FAM129B | 3.95E−77 | −1.006 | 0.204 | 0.221 | 4.50E−73 |
| MRPL1 | 4.99E−77 | −0.464 | 0.343 | 0.209 | 5.69E−73 |
| CDKN2A | 6.20E−77 | −1.166 | 0.084 | 0.235 | 7.07E−73 |
| VPS35 | 1.04E−76 | −0.455 | 0.344 | 0.25 | 1.19E−72 |
| PARP9 | 1.08E−76 | −0.325 | 0.292 | 0.141 | 1.23E−72 |
| NRD1 | 1.43E−76 | −0.305 | 0.393 | 0.221 | 1.63E−72 |
| POLR2H | 2.08E−76 | −0.351 | 0.359 | 0.229 | 2.37E−72 |
| LYRM2 | 2.14E−76 | −0.267 | 0.345 | 0.169 | 2.44E−72 |
| BAZ1A | 2.28E−76 | −0.252 | 0.411 | 0.198 | 2.60E−72 |
| TAF1D | 3.78E−76 | −1.153 | 0.207 | 0.313 | 4.31E−72 |
| DNMT1 | 5.32E−76 | −0.749 | 0.325 | 0.269 | 6.07E−72 |
| PIK3C2A | 5.91E−76 | −0.287 | 0.302 | 0.16 | 6.73E−72 |
| SNRPA1 | 7.32E−76 | −0.424 | 0.218 | 0.12 | 8.35E−72 |
| MACF1 | 8.52E−76 | −0.486 | 0.37 | 0.23 | 9.71E−72 |
| FGFR1OP2 | 9.16E−76 | −0.347 | 0.357 | 0.2 | 1.04E−71 |
| TARS | 2.01E−75 | −0.833 | 0.315 | 0.322 | 2.29E−71 |
| DDAH2 | 3.01E−75 | −0.284 | 0.447 | 0.257 | 3.43E−71 |
| NUDT21 | 3.21E−75 | −0.339 | 0.295 | 0.171 | 3.66E−71 |
| NIPBL | 3.35E−75 | −0.499 | 0.452 | 0.308 | 3.82E−71 |
| MRPL15 | 4.44E−75 | −0.273 | 0.297 | 0.152 | 5.06E−71 |
| RCN2 | 4.64E−75 | −0.361 | 0.344 | 0.233 | 5.29E−71 |
| SEL1L | 6.36E−75 | −0.258 | 0.392 | 0.212 | 7.25E−71 |
| WDR45B | 6.95E−75 | −0.540 | 0.328 | 0.246 | 7.92E−71 |
| FAM210B | 7.18E−75 | −0.395 | 0.329 | 0.221 | 8.18E−71 |
| PFDN4 | 8.98E−75 | −0.822 | 0.31 | 0.301 | 1.02E−70 |
| RND3 | 1.15E−74 | −0.894 | 0.249 | 0.208 | 1.31E−70 |
| FAM136A | 1.31E−74 | −0.332 | 0.264 | 0.155 | 1.49E−70 |
| PKN2 | 1.38E−74 | −0.286 | 0.334 | 0.181 | 1.57E−70 |
| DKC1 | 1.68E−74 | −0.664 | 0.311 | 0.283 | 1.91E−70 |
| APOL6 | 1.80E−74 | −0.662 | 0.215 | 0.153 | 2.05E−70 |
| SPCS2 | 1.88E−74 | −0.646 | 0.232 | 0.26 | 2.14E−70 |
| ANXA1 | 2.17E−74 | −1.600 | 0.043 | 0.257 | 2.48E−70 |
| DDOST | 3.20E−74 | −0.263 | 0.316 | 0.186 | 3.64E−70 |
| NOLC1 | 4.13E−74 | −0.348 | 0.402 | 0.259 | 4.71E−70 |
| FYTTD1 | 5.30E−74 | −0.268 | 0.395 | 0.223 | 6.04E−70 |
| 11-Sep | 5.65E−74 | −0.738 | 0.306 | 0.259 | 6.44E−70 |
| ROCK1 | 6.86E−74 | −0.776 | 0.256 | 0.291 | 7.82E−70 |
| ACTL6A | 9.17E−74 | −0.459 | 0.281 | 0.19 | 1.05E−69 |
| ASS1 | 1.17E−73 | −0.901 | 0.003 | 0.222 | 1.33E−69 |
| RBBP4 | 1.48E−73 | −0.471 | 0.306 | 0.229 | 1.69E−69 |
| RIF1 | 1.51E−73 | −0.740 | 0.333 | 0.28 | 1.72E−69 |
| CETN2 | 1.58E−73 | −0.325 | 0.403 | 0.231 | 1.80E−69 |
| ARHGDIA | 2.27E−73 | −0.431 | 0.357 | 0.262 | 2.59E−69 |
| HUWE1 | 2.88E−73 | −0.794 | 0.244 | 0.265 | 3.29E−69 |
| APRT | 4.32E−73 | −0.461 | 0.316 | 0.233 | 4.92E−69 |
| SSRP1 | 4.65E−73 | −0.627 | 0.285 | 0.256 | 5.29E−69 |
| PABPC3 | 5.29E−73 | −1.061 | 0.08 | 0.257 | 6.03E−69 |
| AHCY | 5.42E−73 | −0.267 | 0.368 | 0.211 | 6.18E−69 |
| MED10 | 8.14E−73 | −0.306 | 0.271 | 0.133 | 9.28E−69 |
| MTHFD2 | 8.80E−73 | −0.288 | 0.501 | 0.313 | 1.00E−68 |
| SRSF6 | 9.23E−73 | −0.264 | 0.369 | 0.205 | 1.05E−68 |
| VBP1 | 1.03E−72 | −0.362 | 0.432 | 0.284 | 1.17E−68 |
| BRIX1 | 1.25E−72 | −0.497 | 0.279 | 0.211 | 1.43E−68 |
| TRIM44 | 2.10E−72 | −1.198 | 0.147 | 0.208 | 2.39E−68 |
| MCM4 | 2.34E−72 | −0.861 | 0.346 | 0.298 | 2.67E−68 |
| USP14 | 2.39E−72 | −0.567 | 0.298 | 0.265 | 2.72E−68 |
| FAM32A | 3.46E−72 | −0.323 | 0.476 | 0.313 | 3.94E−68 |
| LINC00657 | 4.44E−72 | −0.406 | 0.446 | 0.313 | 5.06E−68 |
| TMX1 | 5.20E−72 | −0.330 | 0.313 | 0.175 | 5.92E−68 |
| OIP5-AS1 | 5.75E−72 | −0.271 | 0.301 | 0.162 | 6.56E−68 |
| NCBP2 | 6.69E−72 | −0.545 | 0.356 | 0.289 | 7.63E−68 |
| SLC44A2 | 6.79E−72 | −0.571 | 0.241 | 0.189 | 7.74E−68 |
| GALNT1 | 7.52E−72 | −0.296 | 0.377 | 0.215 | 8.57E−68 |
| HMGA1 | 7.63E−72 | −0.415 | 0.375 | 0.249 | 8.70E−68 |
| DDB1 | 7.66E−72 | −0.402 | 0.24 | 0.16 | 8.73E−68 |
| XPO1 | 1.10E−71 | −0.313 | 0.382 | 0.234 | 1.26E−67 |
| PUS7L | 1.41E−71 | −0.375 | 0.248 | 0.122 | 1.61E−67 |
| NIPSNAP1 | 2.36E−71 | −0.282 | 0.293 | 0.164 | 2.69E−67 |
| ZNF292 | 3.04E−71 | −0.471 | 0.285 | 0.173 | 3.46E−67 |
| SOX4 | 3.30E−71 | −0.601 | 0.579 | 0.472 | 3.77E−67 |
| SKIL | 3.41E−71 | −0.311 | 0.365 | 0.207 | 3.89E−67 |
| CGGBP1 | 4.07E−71 | −0.275 | 0.317 | 0.191 | 4.64E−67 |
| TOP2B | 6.60E−71 | −0.428 | 0.308 | 0.209 | 7.53E−67 |
| TRIP12 | 1.01E−70 | −0.310 | 0.346 | 0.205 | 1.15E−66 |
| HNRNPUL1 | 1.11E−70 | −0.434 | 0.363 | 0.278 | 1.27E−66 |
| PUF60 | 1.13E−70 | −0.304 | 0.31 | 0.192 | 1.29E−66 |
| PLA2G16 | 1.78E−70 | −0.474 | 0.234 | 0.18 | 2.03E−66 |
| DANCR | 1.79E−70 | −0.962 | 0.184 | 0.334 | 2.04E−66 |
| MAP4 | 2.87E−70 | −0.550 | 0.285 | 0.207 | 3.27E−66 |
| MCUR1 | 4.61E−70 | −0.407 | 0.373 | 0.25 | 5.25E−66 |
| USP1 | 4.72E−70 | −0.551 | 0.481 | 0.36 | 5.38E−66 |
| GARS | 5.08E−70 | −0.406 | 0.378 | 0.252 | 5.79E−66 |
| LONP2 | 6.40E−70 | −0.348 | 0.299 | 0.189 | 7.30E−66 |
| SMARCA2 | 6.56E−70 | −0.405 | 0.277 | 0.159 | 7.48E−66 |
| ABCF1 | 7.23E−70 | −0.279 | 0.42 | 0.243 | 8.24E−66 |
| GCC2 | 7.51E−70 | −0.356 | 0.409 | 0.237 | 8.56E−66 |
| SF1 | 8.83E−70 | −0.447 | 0.269 | 0.188 | 1.01E−65 |
| NOP16 | 1.16E−69 | −0.340 | 0.279 | 0.168 | 1.32E−65 |
| STAG2 | 1.40E−69 | −0.327 | 0.352 | 0.217 | 1.60E−65 |
| MARCKSL1 | 1.79E−69 | −0.407 | 0.355 | 0.256 | 2.04E−65 |
| S100A9 | 2.47E−69 | −1.485 | 0.035 | 0.287 | 2.81E−65 |
| LSM14A | 2.57E−69 | −0.540 | 0.358 | 0.293 | 2.93E−65 |
| NACA2 | 2.78E−69 | −0.273 | 0.309 | 0.186 | 3.17E−65 |
| PSMD1 | 3.47E−69 | −0.369 | 0.373 | 0.233 | 3.96E−65 |
| NTPCR | 4.02E−69 | −3.595 | 0.352 | 0.217 | 4.58E−65 |
| LRPPRC | 5.56E−69 | −0.269 | 0.475 | 0.29 | 6.34E−65 |
| TNPO1 | 6.62E−69 | −0.315 | 0.316 | 0.184 | 7.54E−65 |
| CKS2 | 7.39E−69 | −0.328 | 0.458 | 0.261 | 8.42E−65 |
| RPS12 | 1.34E−68 | −0.465 | 0.997 | 0.965 | 1.53E−64 |
| SLC25A36 | 2.09E−68 | −0.384 | 0.277 | 0.175 | 2.38E−64 |
| NUDCD2 | 2.29E−68 | −0.295 | 0.271 | 0.159 | 2.61E−64 |
| PCM1 | 2.46E−68 | −0.554 | 0.34 | 0.258 | 2.80E−64 |
| THOC7 | 3.65E−68 | −0.379 | 0.39 | 0.265 | 4.16E−64 |
| CTSH | 5.31E−68 | −0.277 | 0.234 | 0.11 | 6.05E−64 |
| SREK1 | 7.04E−68 | −0.272 | 0.398 | 0.211 | 8.02E−64 |
| RB1CC1 | 7.93E−68 | −0.760 | 0.362 | 0.338 | 9.04E−64 |
| PANK3 | 9.97E−68 | −0.341 | 0.282 | 0.175 | 1.14E−63 |
| TWISTNB | 1.04E−67 | −0.258 | 0.276 | 0.132 | 1.18E−63 |
| ANP32E | 1.11E−67 | −0.821 | 0.199 | 0.202 | 1.27E−63 |
| DESI2 | 1.68E−67 | −0.382 | 0.255 | 0.147 | 1.91E−63 |
| RPL21 | 2.24E−67 | −0.710 | 0.215 | 0.223 | 2.55E−63 |
| UBE2L3 | 2.88E−67 | −0.388 | 0.282 | 0.197 | 3.28E−63 |
| RANBP2 | 4.21E−67 | −0.735 | 0.199 | 0.239 | 4.80E−63 |
| DPM1 | 5.81E−67 | −0.299 | 0.346 | 0.224 | 6.62E−63 |
| MRPL12 | 8.96E−67 | −0.448 | 0.325 | 0.252 | 1.02E−62 |
| DERL1 | 1.03E−66 | −0.360 | 0.255 | 0.17 | 1.17E−62 |
| WHSC1L1 | 1.15E−66 | −0.316 | 0.339 | 0.211 | 1.31E−62 |
| CLIC1 | 1.22E−66 | −0.260 | 0.364 | 0.209 | 1.39E−62 |
| MPHOSPH6 | 1.36E−66 | −0.538 | 0.343 | 0.29 | 1.55E−62 |
| BSG | 2.02E−66 | −0.348 | 0.331 | 0.217 | 2.30E−62 |
| EWSR1 | 2.04E−66 | −0.431 | 0.273 | 0.199 | 2.33E−62 |
| CDV3 | 2.42E−66 | −0.630 | 0.258 | 0.223 | 2.75E−62 |
| TFAM | 2.47E−66 | −0.386 | 0.366 | 0.262 | 2.82E−62 |
| HTATSF1 | 4.95E−66 | −0.433 | 0.367 | 0.259 | 5.65E−62 |
| IRX3 | 6.94E−66 | −0.931 | 0.003 | 0.202 | 7.91E−62 |
| TFRC | 7.39E−66 | −0.927 | 0.2 | 0.281 | 8.42E−62 |
| PARP14 | 1.42E−65 | −0.285 | 0.258 | 0.121 | 1.62E−61 |
| FRG1 | 1.68E−65 | −0.327 | 0.38 | 0.257 | 1.91E−61 |
| SUPT5H | 2.22E−65 | −0.419 | 0.27 | 0.184 | 2.53E−61 |
| SOX9 | 2.55E−65 | −0.263 | 0.246 | 0.12 | 2.91E−61 |
| TRIB1 | 2.66E−65 | −0.654 | 0.296 | 0.253 | 3.03E−61 |
| TXNL4A | 3.86E−65 | −0.446 | 0.268 | 0.227 | 4.40E−61 |
| DCUN1D5 | 8.34E−65 | −0.407 | 0.26 | 0.19 | 9.50E−61 |
| SMC1A | 8.77E−65 | −0.423 | 0.284 | 0.181 | 9.99E−61 |
| STARD7 | 8.86E−65 | −0.641 | 0.228 | 0.227 | 1.01E−60 |
| PNO1 | 8.89E−65 | −0.289 | 0.299 | 0.176 | 1.01E−60 |
| HMGN3 | 1.15E−64 | −0.425 | 0.276 | 0.183 | 1.31E−60 |
| NEAT1 | 1.26E−64 | −1.473 | 0.27 | 0.426 | 1.44E−60 |
| TXNL1 | 1.35E−64 | −0.313 | 0.304 | 0.196 | 1.54E−60 |
| ZBTB43 | 1.43E−64 | −0.900 | 0.159 | 0.162 | 1.63E−60 |
| MT-ND6 | 1.62E−64 | −0.390 | 0.346 | 0.225 | 1.85E−60 |
| CUL4A | 1.72E−64 | −0.479 | 0.222 | 0.166 | 1.96E−60 |
| TSG101 | 1.81E−64 | −0.269 | 0.375 | 0.237 | 2.06E−60 |
| SMARCC1 | 2.12E−64 | −0.733 | 0.34 | 0.32 | 2.42E−60 |
| ARCN1 | 2.16E−64 | −0.285 | 0.309 | 0.178 | 2.46E−60 |
| SPTY2D1 | 2.17E−64 | −0.457 | 0.324 | 0.24 | 2.47E−60 |
| MAPK1IP1L | 2.38E−64 | −0.287 | 0.272 | 0.16 | 2.71E−60 |
| RRP1B | 2.40E−64 | −0.300 | 0.254 | 0.143 | 2.74E−60 |
| EIF3B | 2.67E−64 | −0.250 | 0.307 | 0.178 | 3.04E−60 |
| CHD8 | 3.51E−64 | −0.291 | 0.283 | 0.154 | 4.00E−60 |
| EIF4EBP1 | 4.27E−64 | −0.788 | 0.21 | 0.262 | 4.87E−60 |
| ATXN10 | 7.91E−64 | −0.274 | 0.323 | 0.197 | 9.01E−60 |
| NEMF | 1.04E−63 | −0.493 | 0.262 | 0.17 | 1.19E−59 |
| BIRC6 | 1.40E−63 | −0.278 | 0.294 | 0.17 | 1.59E−59 |
| NASP | 1.56E−63 | −0.562 | 0.353 | 0.274 | 1.77E−59 |
| KAT6B | 2.18E−63 | −0.437 | 0.333 | 0.219 | 2.49E−59 |
| IFI30 | 2.91E−63 | −0.388 | 0.255 | 0.159 | 3.32E−59 |
| GNL2 | 3.07E−63 | −0.439 | 0.295 | 0.189 | 3.49E−59 |
| MRPS28 | 3.77E−63 | −0.278 | 0.391 | 0.251 | 4.29E−59 |
| DCAF13 | 3.85E−63 | −0.724 | 0.176 | 0.203 | 4.39E−59 |
| UTP11L | 4.64E−63 | −0.422 | 0.305 | 0.203 | 5.29E−59 |
| HK1 | 4.76E−63 | −0.286 | 0.275 | 0.179 | 5.42E−59 |
| MTRNR2L13 | 5.17E−63 | −1.127 | 0.085 | 0.178 | 5.90E−59 |
| HTATIP2 | 5.23E−63 | −0.502 | 0.189 | 0.132 | 5.96E−59 |
| ITSN2 | 5.60E−63 | −0.275 | 0.333 | 0.173 | 6.38E−59 |
| IGF1R | 5.81E−63 | −2.324 | 0.013 | 0.179 | 6.63E−59 |
| GPBP1L1 | 6.17E−63 | −0.328 | 0.279 | 0.179 | 7.03E−59 |
| GAPDH | 6.60E−63 | −0.306 | 0.988 | 0.855 | 7.52E−59 |
| RCN1 | 6.66E−63 | −0.562 | 0.257 | 0.204 | 7.60E−59 |
| PNPT1 | 6.91E−63 | −0.268 | 0.272 | 0.153 | 7.87E−59 |
| IVNS1ABP | 7.33E−63 | −0.355 | 0.305 | 0.178 | 8.36E−59 |
| CYP4Z1 | 1.20E−62 | −1.153 | 0.004 | 0.198 | 1.37E−58 |
| BMS1 | 1.54E−62 | −0.286 | 0.308 | 0.173 | 1.76E−58 |
| FAM211A | 1.55E−62 | −0.424 | 0.331 | 0.234 | 1.76E−58 |
| CDC16 | 1.60E−62 | −0.475 | 0.277 | 0.183 | 1.82E−58 |
| ZNF148 | 1.72E−62 | −0.253 | 0.261 | 0.136 | 1.96E−58 |
| STK3 | 1.78E−62 | −0.338 | 0.311 | 0.195 | 2.03E−58 |
| S100A8 | 2.48E−62 | −1.502 | 0.039 | 0.263 | 2.83E−58 |
| CDK12 | 4.24E−62 | −0.843 | 0.231 | 0.25 | 4.83E−58 |
| CREB1 | 4.58E−62 | −0.259 | 0.246 | 0.13 | 5.22E−58 |
| CHCHD7 | 5.22E−62 | −0.281 | 0.222 | 0.12 | 5.95E−58 |
| RIOK3 | 5.75E−62 | −0.504 | 0.25 | 0.204 | 6.56E−58 |
| NAA50 | 6.13E−62 | −0.580 | 0.245 | 0.217 | 6.99E−58 |
| TMPO | 7.18E−62 | −0.383 | 0.366 | 0.22 | 8.18E−58 |
| MSL1 | 7.61E−62 | −0.426 | 0.324 | 0.243 | 8.67E−58 |
| RDH11 | 8.69E−62 | −0.279 | 0.339 | 0.203 | 9.90E−58 |
| RNF168 | 8.91E−62 | −0.533 | 0.248 | 0.182 | 1.02E−57 |
| SKIV2L2 | 1.07E−61 | −0.329 | 0.209 | 0.111 | 1.22E−57 |
| KHSRP | 1.13E−61 | −0.455 | 0.25 | 0.191 | 1.29E−57 |
| SRPK1 | 1.26E−61 | −0.256 | 0.339 | 0.198 | 1.43E−57 |
| PDS5A | 1.46E−61 | −0.410 | 0.299 | 0.198 | 1.66E−57 |
| HMGN2 | 1.88E−61 | −0.320 | 0.573 | 0.385 | 2.14E−57 |
| ESF1 | 2.17E−61 | −0.613 | 0.205 | 0.192 | 2.48E−57 |
| LPGAT1 | 2.31E−61 | −0.563 | 0.217 | 0.186 | 2.63E−57 |
| DPYSL2 | 2.74E−61 | −0.439 | 0.239 | 0.163 | 3.12E−57 |
| GLYATL2 | 3.21E−61 | −2.296 | 0 | 0.174 | 3.66E−57 |
| NUDCD1 | 4.14E−61 | −0.774 | 0.194 | 0.243 | 4.71E−57 |
| SAFB | 7.40E−61 | −0.253 | 0.246 | 0.129 | 8.43E−57 |
| NPC2 | 7.53E−61 | −0.745 | 0.258 | 0.281 | 8.58E−57 |
| ATG3 | 8.25E−61 | −0.439 | 0.251 | 0.174 | 9.40E−57 |
| RN7SK | 8.41E−61 | −0.620 | 0.259 | 0.201 | 9.59E−57 |
| SPP1 | 9.66E−61 | −1.862 | 0.027 | 0.234 | 1.10E−56 |
| PUM2 | 1.04E−60 | −0.498 | 0.244 | 0.19 | 1.18E−56 |
| C3 | 1.27E−60 | −1.265 | 0.05 | 0.137 | 1.45E−56 |
| ARHGAP5 | 2.02E−60 | −0.742 | 0.233 | 0.247 | 2.30E−56 |
| TUBA1A | 2.71E−60 | −1.491 | 0.064 | 0.288 | 3.09E−56 |
| SMC3 | 3.28E−60 | −0.325 | 0.356 | 0.216 | 3.74E−56 |
| IPO9 | 4.35E−60 | −0.291 | 0.221 | 0.127 | 4.96E−56 |
| MT2A | 4.54E−60 | −0.898 | 0.353 | 0.329 | 5.18E−56 |
| PSMD3 | 5.14E−60 | −0.304 | 0.317 | 0.218 | 5.86E−56 |
| TPD52L1 | 5.19E−60 | −0.639 | 0.21 | 0.182 | 5.92E−56 |
| CASC3 | 7.31E−60 | −0.547 | 0.213 | 0.212 | 8.33E−56 |
| FARP1 | 9.65E−60 | −0.473 | 0.24 | 0.176 | 1.10E−55 |
| CBX1 | 1.16E−59 | −0.298 | 0.335 | 0.208 | 1.32E−55 |
| PPP2R1A | 1.43E−59 | −0.339 | 0.295 | 0.197 | 1.63E−55 |
| CKAP5 | 1.59E−59 | −0.347 | 0.242 | 0.146 | 1.81E−55 |
| HMGB3 | 1.86E−59 | −0.805 | 0.198 | 0.252 | 2.12E−55 |
| RPL35 | 2.28E−59 | −0.320 | 0.994 | 0.932 | 2.60E−55 |
| PAFAH1B2 | 2.44E−59 | −0.564 | 0.2 | 0.189 | 2.78E−55 |
| MEF2A | 2.49E−59 | −0.776 | 0.213 | 0.243 | 2.84E−55 |
| GRB7 | 4.64E−59 | −0.819 | 0.003 | 0.183 | 5.29E−55 |
| BOD1L1 | 4.94E−59 | −0.428 | 0.279 | 0.181 | 5.63E−55 |
| VIM | 5.34E−59 | −1.525 | 0.068 | 0.223 | 6.09E−55 |
| ZHX1 | 7.24E−59 | −0.531 | 0.231 | 0.178 | 8.25E−55 |
| EMD | 8.05E−59 | −0.339 | 0.238 | 0.162 | 9.17E−55 |
| BRD4 | 8.80E−59 | −0.681 | 0.277 | 0.27 | 1.00E−54 |
| PNMT | 9.72E−59 | −1.045 | 0.003 | 0.183 | 1.11E−54 |
| STIP1 | 1.07E−58 | −0.499 | 0.267 | 0.212 | 1.22E−54 |
| VTA1 | 1.07E−58 | −0.320 | 0.268 | 0.172 | 1.22E−54 |
| LIMS1 | 1.09E−58 | −0.487 | 0.209 | 0.146 | 1.24E−54 |
| NFE2L1 | 1.16E−58 | −0.308 | 0.28 | 0.176 | 1.32E−54 |
| PPP1CC | 2.33E−58 | −0.346 | 0.304 | 0.217 | 2.66E−54 |
| PTMA | 2.39E−58 | −0.325 | 0.987 | 0.882 | 2.72E−54 |
| MIB1 | 2.58E−58 | −0.453 | 0.234 | 0.187 | 2.94E−54 |
| SMCHD1 | 3.09E−58 | −0.869 | 0.162 | 0.269 | 3.52E−54 |
| CCDC91 | 3.64E−58 | −0.355 | 0.323 | 0.212 | 4.15E−54 |
| LSM2 | 3.95E−58 | −0.431 | 0.334 | 0.254 | 4.51E−54 |
| IARS | 4.50E−58 | −0.252 | 0.311 | 0.198 | 5.12E−54 |
| ADK | 4.79E−58 | −0.550 | 0.272 | 0.249 | 5.46E−54 |
| DDX42 | 5.04E−58 | −0.291 | 0.297 | 0.183 | 5.74E−54 |
| CBR1 | 5.27E−58 | −0.772 | 0.002 | 0.177 | 6.01E−54 |
| DDX27 | 5.59E−58 | −0.291 | 0.302 | 0.177 | 6.37E−54 |
| TIMM44 | 6.18E−58 | −0.535 | 0.231 | 0.188 | 7.05E−54 |
| GAR1 | 6.72E−58 | −0.321 | 0.225 | 0.129 | 7.66E−54 |
| DARS | 7.05E−58 | −0.398 | 0.276 | 0.193 | 8.03E−54 |
| SDC2 | 7.43E−58 | −1.378 | 0.001 | 0.17 | 8.47E−54 |
| FPGS | 7.54E−58 | −0.477 | 0.144 | 0.101 | 8.60E−54 |
| EIF4E2 | 8.41E−58 | −0.347 | 0.301 | 0.201 | 9.59E−54 |
| UBBP4 | 8.48E−58 | −0.288 | 0.219 | 0.127 | 9.66E−54 |
| MSMO1 | 1.21E−57 | −0.463 | 0.314 | 0.234 | 1.38E−53 |
| YIF1A | 1.33E−57 | −0.308 | 0.228 | 0.141 | 1.52E−53 |
| SNX5 | 1.40E−57 | −0.273 | 0.272 | 0.162 | 1.60E−53 |
| BAZ2B | 2.06E−57 | −0.299 | 0.332 | 0.186 | 2.35E−53 |
| C17orf89 | 4.47E−57 | −0.659 | 0.214 | 0.256 | 5.10E−53 |
| SNHG16 | 6.40E−57 | −0.453 | 0.28 | 0.245 | 7.29E−53 |
| RDX | 6.95E−57 | −0.951 | 0.07 | 0.264 | 7.92E−53 |
| CYP1B1 | 7.97E−57 | −1.543 | 0.002 | 0.161 | 9.08E−53 |
| TOMM22 | 8.06E−57 | −0.445 | 0.265 | 0.209 | 9.19E−53 |
| TAOK1 | 8.37E−57 | −0.254 | 0.335 | 0.177 | 9.54E−53 |
| TIA1 | 1.13E−56 | −0.488 | 0.226 | 0.18 | 1.29E−52 |
| CISD2 | 1.19E−56 | −0.270 | 0.25 | 0.151 | 1.36E−52 |
| C8orf33 | 1.23E−56 | −0.469 | 0.306 | 0.255 | 1.40E−52 |
| RRP15 | 1.39E−56 | −0.315 | 0.307 | 0.187 | 1.59E−52 |
| NUDT5 | 1.76E−56 | −0.469 | 0.242 | 0.201 | 2.01E−52 |
| EBAG9 | 2.58E−56 | −0.482 | 0.282 | 0.215 | 2.94E−52 |
| UTRN | 3.22E−56 | −0.753 | 0.209 | 0.194 | 3.66E−52 |
| MT-ND4 | 3.81E−56 | −0.494 | 0.992 | 0.887 | 4.34E−52 |
| OXR1 | 4.42E−56 | −0.531 | 0.206 | 0.176 | 5.04E−52 |
| TPP2 | 5.96E−56 | −0.315 | 0.227 | 0.146 | 6.79E−52 |
| PPIF | 6.24E−56 | −0.295 | 0.29 | 0.186 | 7.11E−52 |
| DCUN1D1 | 6.37E−56 | −0.332 | 0.218 | 0.142 | 7.26E−52 |
| CEP70 | 7.65E−56 | −0.519 | 0.162 | 0.124 | 8.72E−52 |
| SMARCA4 | 8.82E−56 | −0.510 | 0.225 | 0.18 | 1.01E−51 |
| WNK1 | 1.01E−55 | −0.471 | 0.253 | 0.194 | 1.16E−51 |
| XRN1 | 1.03E−55 | −0.340 | 0.215 | 0.132 | 1.18E−51 |
| NUP54 | 1.28E−55 | −0.372 | 0.199 | 0.133 | 1.46E−51 |
| PTK2 | 1.58E−55 | −0.266 | 0.322 | 0.209 | 1.80E−51 |
| NKTR | 1.80E−55 | −0.647 | 0.198 | 0.186 | 2.05E−51 |
| GATAD2A | 1.98E−55 | −0.340 | 0.239 | 0.144 | 2.25E−51 |
| CNOT1 | 2.26E−55 | −0.462 | 0.2 | 0.167 | 2.57E−51 |
| AFG3L2 | 2.38E−55 | −0.750 | 0.193 | 0.268 | 2.71E−51 |
| ECT2 | 2.44E−55 | −0.333 | 0.268 | 0.148 | 2.78E−51 |
| SNRNP200 | 2.52E−55 | −0.463 | 0.181 | 0.151 | 2.88E−51 |
| ARHGAP35 | 2.53E−55 | −0.414 | 0.24 | 0.18 | 2.89E−51 |
| NDUFAF2 | 2.82E−55 | −0.335 | 0.238 | 0.144 | 3.21E−51 |
| CHD9 | 2.85E−55 | −0.415 | 0.216 | 0.129 | 3.25E−51 |
| PLP1 | 3.11E−55 | −1.354 | 0.005 | 0.179 | 3.55E−51 |
| NAA10 | 3.46E−55 | −0.425 | 0.185 | 0.141 | 3.95E−51 |
| EIF3D | 3.66E−55 | −0.409 | 0.338 | 0.251 | 4.17E−51 |
| MRPS23 | 5.78E−55 | −0.269 | 0.241 | 0.141 | 6.59E−51 |
| RBPJ | 6.22E−55 | −0.347 | 0.253 | 0.161 | 7.09E−51 |
| ARFGAP3 | 7.88E−55 | −0.387 | 0.264 | 0.171 | 8.98E−51 |
| CTDSP2 | 8.10E−55 | −0.303 | 0.203 | 0.121 | 9.24E−51 |
| ANKRD17 | 8.47E−55 | −0.450 | 0.268 | 0.207 | 9.66E−51 |
| TAF15 | 1.21E−54 | −0.355 | 0.239 | 0.169 | 1.38E−50 |
| UPF2 | 1.29E−54 | −0.942 | 0.13 | 0.245 | 1.47E−50 |
| NBN | 1.38E−54 | −0.519 | 0.308 | 0.268 | 1.58E−50 |
| TFDP1 | 1.87E−54 | −0.574 | 0.191 | 0.176 | 2.13E−50 |
| MGEA5 | 2.49E−54 | −0.520 | 0.265 | 0.224 | 2.84E−50 |
| STX7 | 2.73E−54 | −0.254 | 0.194 | 0.113 | 3.11E−50 |
| DHX36 | 2.97E−54 | −0.548 | 0.259 | 0.229 | 3.39E−50 |
| OPA1 | 3.16E−54 | −0.383 | 0.286 | 0.21 | 3.61E−50 |
| AIF1L | 4.04E−54 | −0.349 | 0.214 | 0.127 | 4.60E−50 |
| SPAG9 | 4.97E−54 | −0.598 | 0.218 | 0.211 | 5.67E−50 |
| ATP13A3 | 5.04E−54 | −0.573 | 0.18 | 0.161 | 5.74E−50 |
| TATDN1 | 5.16E−54 | −0.525 | 0.255 | 0.211 | 5.88E−50 |
| PRRC2B | 5.96E−54 | −0.358 | 0.269 | 0.208 | 6.80E−50 |
| COMMD2 | 9.88E−54 | −0.534 | 0.163 | 0.142 | 1.13E−49 |
| SLC39A14 | 1.12E−53 | −0.305 | 0.169 | 0.1 | 1.27E−49 |
| API5 | 1.49E−53 | −0.268 | 0.229 | 0.128 | 1.70E−49 |
| GS1-251I9.4 | 1.64E−53 | −0.523 | 0.233 | 0.209 | 1.87E−49 |
| PRPF38A | 1.78E−53 | −0.307 | 0.197 | 0.118 | 2.02E−49 |
| CEBPG | 1.91E−53 | −0.335 | 0.323 | 0.227 | 2.18E−49 |
| CAPG | 1.92E−53 | −0.557 | 0.185 | 0.154 | 2.18E−49 |
| GON4L | 1.94E−53 | −0.274 | 0.164 | 0.078 | 2.21E−49 |
| GMPS | 1.97E−53 | −0.281 | 0.274 | 0.167 | 2.25E−49 |
| FXYD5 | 2.19E−53 | −0.606 | 0.149 | 0.152 | 2.50E−49 |
| CSNK1D | 2.39E−53 | −0.260 | 0.272 | 0.169 | 2.72E−49 |
| CHML | 2.71E−53 | −0.821 | 0.119 | 0.183 | 3.09E−49 |
| KPNA2 | 2.78E−53 | −0.603 | 0.192 | 0.173 | 3.16E−49 |
| HIST1H1C | 3.49E−53 | −1.175 | 0.149 | 0.3 | 3.97E−49 |
| ARFGEF1 | 3.51E−53 | −0.295 | 0.244 | 0.148 | 4.01E−49 |
| UBA2 | 4.28E−53 | −0.502 | 0.199 | 0.183 | 4.87E−49 |
| VMA21 | 4.50E−53 | −0.415 | 0.234 | 0.172 | 5.13E−49 |
| ANKRD10 | 5.01E−53 | −0.453 | 0.196 | 0.151 | 5.71E−49 |
| STAU2 | 5.02E−53 | −0.288 | 0.255 | 0.166 | 5.72E−49 |
| USP10 | 5.97E−53 | −0.344 | 0.254 | 0.174 | 6.80E−49 |
| AKT2 | 5.97E−53 | −0.250 | 0.207 | 0.123 | 6.80E−49 |
| COMTD1 | 6.36E−53 | −0.304 | 0.263 | 0.174 | 7.25E−49 |
| ANKRD30B | 6.76E−53 | −1.291 | 0.084 | 0.194 | 7.70E−49 |
| RMRP | 1.39E−52 | −0.869 | 0.141 | 0.211 | 1.59E−48 |
| MRPL50 | 1.95E−52 | −0.265 | 0.215 | 0.124 | 2.22E−48 |
| NCOA2 | 1.96E−52 | −0.292 | 0.216 | 0.111 | 2.23E−48 |
| STK24 | 2.39E−52 | −0.406 | 0.198 | 0.154 | 2.72E−48 |
| EDARADD | 2.45E−52 | −0.830 | 0.097 | 0.195 | 2.79E−48 |
| IFRD2 | 2.51E−52 | −0.766 | 0.11 | 0.155 | 2.86E−48 |
| MTRNR2L7 | 2.64E−52 | −1.319 | 0.032 | 0.187 | 3.01E−48 |
| PDCD4 | 2.79E−52 | −0.304 | 0.271 | 0.167 | 3.18E−48 |
| CPSF2 | 3.22E−52 | −0.407 | 0.257 | 0.179 | 3.67E−48 |
| RP11-304L19.5 | 3.34E−52 | −0.665 | 0.281 | 0.275 | 3.80E−48 |
| MIR4458HG | 3.79E−52 | −0.355 | 0.267 | 0.176 | 4.32E−48 |
| DOCK5 | 3.94E−52 | −0.264 | 0.153 | 0.08 | 4.49E−48 |
| PPP3CA | 6.99E−52 | −0.256 | 0.28 | 0.166 | 7.97E−48 |
| ZNF121 | 9.31E−52 | −0.325 | 0.205 | 0.136 | 1.06E−47 |
| CRNDE | 1.07E−51 | −0.320 | 0.202 | 0.138 | 1.22E−47 |
| ATP6V1A | 1.12E−51 | −0.284 | 0.165 | 0.109 | 1.28E−47 |
| NDUFAF6 | 1.13E−51 | −0.331 | 0.284 | 0.188 | 1.29E−47 |
| RPS18 | 1.41E−51 | −0.461 | 0.996 | 0.941 | 1.60E−47 |
| VEGFA | 1.79E−51 | −0.654 | 0.17 | 0.204 | 2.04E−47 |
| HIPK3 | 1.83E−51 | −0.341 | 0.212 | 0.134 | 2.09E−47 |
| C11orf83 | 2.24E−51 | −0.374 | 0.245 | 0.186 | 2.55E−47 |
| HMGCR | 2.92E−51 | −0.353 | 0.203 | 0.132 | 3.32E−47 |
| C1orf198 | 3.08E−51 | −0.332 | 0.142 | 0.074 | 3.51E−47 |
| MKI67IP | 4.01E−51 | −0.589 | 0.227 | 0.218 | 4.57E−47 |
| GRPEL1 | 4.16E−51 | −0.338 | 0.228 | 0.161 | 4.74E−47 |
| ODC1 | 5.45E−51 | −0.925 | 0.056 | 0.21 | 6.21E−47 |
| LRRCC1 | 7.47E−51 | −0.402 | 0.256 | 0.164 | 8.51E−47 |
| ANP32A | 7.88E−51 | −0.392 | 0.245 | 0.185 | 8.98E−47 |
| PTPMT1 | 8.78E−51 | −0.269 | 0.178 | 0.113 | 1.00E−46 |
| RPS6KB1 | 9.95E−51 | −0.304 | 0.212 | 0.131 | 1.13E−46 |
| ATP2C1 | 1.61E−50 | −0.335 | 0.234 | 0.164 | 1.84E−46 |
| TIMM50 | 1.89E−50 | −0.317 | 0.225 | 0.163 | 2.15E−46 |
| ANKRD36C | 1.96E−50 | −1.438 | 0.184 | 0.239 | 2.23E−46 |
| DYNC1LI2 | 2.01E−50 | −0.430 | 0.234 | 0.177 | 2.29E−46 |
| ESCO1 | 2.02E−50 | −0.356 | 0.213 | 0.138 | 2.31E−46 |
| ATP6V1E1 | 2.15E−50 | −0.315 | 0.273 | 0.182 | 2.45E−46 |
| MTUS1 | 2.42E−50 | −0.444 | 0.23 | 0.163 | 2.76E−46 |
| ASNSD1 | 3.26E−50 | −0.420 | 0.275 | 0.202 | 3.72E−46 |
| TNRC6B | 3.80E−50 | −0.503 | 0.22 | 0.199 | 4.33E−46 |
| IFT57 | 3.94E−50 | −0.774 | 0.15 | 0.179 | 4.49E−46 |
| SEC61A1 | 4.49E−50 | −0.442 | 0.234 | 0.217 | 5.11E−46 |
| QSER1 | 4.87E−50 | −0.652 | 0.19 | 0.227 | 5.55E−46 |
| KMT2C | 6.00E−50 | −0.784 | 0.196 | 0.259 | 6.84E−46 |
| SLC20A1 | 7.25E−50 | −0.322 | 0.273 | 0.182 | 8.27E−46 |
| KMT2A | 8.59E−50 | −0.603 | 0.196 | 0.191 | 9.79E−46 |
| SKA2 | 8.83E−50 | −0.425 | 0.233 | 0.171 | 1.01E−45 |
| FAM60A | 9.42E−50 | −0.436 | 0.234 | 0.19 | 1.07E−45 |
| EIF1B | 9.56E−50 | −0.263 | 0.214 | 0.139 | 1.09E−45 |
| ARHGEF12 | 1.54E−49 | −0.405 | 0.216 | 0.176 | 1.75E−45 |
| MAP3K1 | 2.00E−49 | −0.307 | 0.188 | 0.111 | 2.28E−45 |
| GPNMB | 2.32E−49 | −1.125 | 0.014 | 0.181 | 2.65E−45 |
| USP34 | 2.59E−49 | −0.260 | 0.298 | 0.187 | 2.95E−45 |
| NUP62 | 3.12E−49 | −0.341 | 0.212 | 0.147 | 3.56E−45 |
| FAM91A1 | 3.25E−49 | −0.497 | 0.213 | 0.184 | 3.70E−45 |
| EMP1 | 3.29E−49 | −1.061 | 0.152 | 0.331 | 3.74E−45 |
| CYP51A1 | 3.68E−49 | −0.273 | 0.152 | 0.076 | 4.20E−45 |
| MTRNR2L5 | 6.05E−49 | −1.057 | 0.028 | 0.179 | 6.90E−45 |
| CCNG2 | 6.30E−49 | −0.757 | 0.193 | 0.226 | 7.19E−45 |
| ORC3 | 6.58E−49 | −0.320 | 0.184 | 0.115 | 7.50E−45 |
| HYOU1 | 6.95E−49 | −0.274 | 0.218 | 0.147 | 7.92E−45 |
| NCOA7 | 7.05E−49 | −0.442 | 0.343 | 0.225 | 8.04E−45 |
| SPIN1 | 9.32E−49 | −0.390 | 0.205 | 0.159 | 1.06E−44 |
| RCAN1 | 1.40E−48 | −0.538 | 0.141 | 0.129 | 1.59E−44 |
| FARSA | 1.47E−48 | −0.356 | 0.184 | 0.161 | 1.68E−44 |
| KLK6 | 3.63E−48 | −1.234 | 0 | 0.139 | 4.14E−44 |
| NUP153 | 4.10E−48 | −0.464 | 0.154 | 0.131 | 4.67E−44 |
| SNRNP70 | 4.18E−48 | −0.450 | 0.261 | 0.209 | 4.76E−44 |
| EMC2 | 4.33E−48 | −0.471 | 0.237 | 0.212 | 4.94E−44 |
| RPS19BP1 | 4.34E−48 | −0.344 | 0.196 | 0.148 | 4.95E−44 |
| MAP1B | 6.90E−48 | −0.843 | 0 | 0.138 | 7.87E−44 |
| FAM208B | 7.60E−48 | −0.663 | 0.203 | 0.231 | 8.66E−44 |
| FLNA | 8.88E−48 | −0.793 | 0.188 | 0.237 | 1.01E−43 |
| PLD3 | 9.31E−48 | −0.270 | 0.217 | 0.137 | 1.06E−43 |
| NUCB1 | 9.58E−48 | −0.343 | 0.214 | 0.16 | 1.09E−43 |
| YES1 | 1.05E−47 | −0.654 | 0.212 | 0.24 | 1.20E−43 |
| CCNL2 | 1.24E−47 | −0.339 | 0.194 | 0.122 | 1.41E−43 |
| FSTL1 | 1.25E−47 | −1.013 | 0.001 | 0.143 | 1.42E−43 |
| STK4 | 1.33E−47 | −0.262 | 0.238 | 0.138 | 1.51E−43 |
| UBAP2 | 1.35E−47 | −0.642 | 0.192 | 0.19 | 1.54E−43 |
| POLR2F | 1.63E−47 | −0.677 | 0.174 | 0.239 | 1.86E−43 |
| EML4 | 1.84E−47 | −0.364 | 0.211 | 0.141 | 2.10E−43 |
| HELZ | 2.00E−47 | −0.357 | 0.174 | 0.129 | 2.28E−43 |
| CD74 | 2.14E−47 | −0.808 | 0.244 | 0.223 | 2.44E−43 |
| RALBP1 | 2.53E−47 | −0.595 | 0.23 | 0.253 | 2.88E−43 |
| STARD3 | 2.65E−47 | −0.656 | 0.151 | 0.211 | 3.02E−43 |
| NUP88 | 3.16E−47 | −0.266 | 0.189 | 0.11 | 3.60E−43 |
| SNHG1 | 3.38E−47 | −0.574 | 0.222 | 0.225 | 3.85E−43 |
| STAC2 | 3.41E−47 | −1.104 | 0.002 | 0.145 | 3.89E−43 |
| IMMT | 5.54E−47 | −0.410 | 0.196 | 0.163 | 6.32E−43 |
| TGFBR1 | 6.38E−47 | −0.945 | 0.042 | 0.202 | 7.27E−43 |
| IRAK1 | 6.55E−47 | −0.506 | 0.16 | 0.145 | 7.46E−43 |
| MED13L | 8.22E−47 | −0.260 | 0.159 | 0.095 | 9.36E−43 |
| MFN1 | 9.45E−47 | −0.255 | 0.184 | 0.112 | 1.08E−42 |
| HEATR1 | 1.02E−46 | −0.492 | 0.182 | 0.154 | 1.16E−42 |
| AGFG1 | 1.23E−46 | −0.319 | 0.193 | 0.12 | 1.41E−42 |
| COMMD1 | 1.42E−46 | −0.321 | 0.182 | 0.126 | 1.61E−42 |
| PLIN2 | 1.45E−46 | −0.886 | 0.151 | 0.169 | 1.65E−42 |
| SLC35F5 | 2.32E−46 | −0.355 | 0.149 | 0.107 | 2.65E−42 |
| BIRC2 | 2.66E−46 | −0.495 | 0.215 | 0.181 | 3.03E−42 |
| CCDC124 | 2.70E−46 | −0.645 | 0.159 | 0.227 | 3.08E−42 |
| KIAA2026 | 2.75E−46 | −0.256 | 0.205 | 0.11 | 3.13E−42 |
| RECQL | 2.78E−46 | −0.336 | 0.218 | 0.135 | 3.16E−42 |
| REV3L | 3.21E−46 | −0.395 | 0.162 | 0.111 | 3.66E−42 |
| NUP50 | 4.45E−46 | −0.453 | 0.101 | 0.109 | 5.08E−42 |
| VPS4B | 4.54E−46 | −0.634 | 0.231 | 0.253 | 5.17E−42 |
| IMPDH2 | 4.99E−46 | −0.713 | 0.1 | 0.224 | 5.69E−42 |
| STAG1 | 5.19E−46 | −0.531 | 0.161 | 0.154 | 5.92E−42 |
| HERPUD1 | 5.82E−46 | −0.383 | 0.244 | 0.195 | 6.64E−42 |
| CYR61 | 7.26E−46 | −0.925 | 0.003 | 0.144 | 8.27E−42 |
| ELOVL6 | 7.31E−46 | −0.608 | 0.125 | 0.12 | 8.33E−42 |
| TYMP | 7.70E−46 | −0.341 | 0.163 | 0.103 | 8.77E−42 |
| CEBPD | 9.86E−46 | −0.912 | 0.068 | 0.181 | 1.12E−41 |
| DST | 1.08E−45 | −0.259 | 0.385 | 0.237 | 1.23E−41 |
| COMMD9 | 1.09E−45 | −0.277 | 0.136 | 0.078 | 1.24E−41 |
| ABCE1 | 1.30E−45 | −0.313 | 0.232 | 0.156 | 1.48E−41 |
| PSMG2 | 1.43E−45 | −0.465 | 0.232 | 0.219 | 1.63E−41 |
| ANKRD11 | 1.53E−45 | −0.787 | 0.125 | 0.188 | 1.75E−41 |
| NSUN2 | 1.65E−45 | −0.386 | 0.18 | 0.153 | 1.89E−41 |
| PCNT | 1.91E−45 | −0.449 | 0.171 | 0.132 | 2.17E−41 |
| GGH | 2.21E−45 | −0.686 | 0 | 0.132 | 2.52E−41 |
| CDC40 | 2.44E−45 | −0.263 | 0.161 | 0.09 | 2.78E−41 |
| TOPBP1 | 2.86E−45 | −0.373 | 0.164 | 0.122 | 3.26E−41 |
| SBNO1 | 2.94E−45 | −0.394 | 0.209 | 0.159 | 3.36E−41 |
| JARID2 | 3.85E−45 | −0.330 | 0.131 | 0.077 | 4.38E−41 |
| SDAD1 | 3.95E−45 | −0.322 | 0.241 | 0.17 | 4.50E−41 |
| MRFAP1L1 | 4.82E−45 | −0.350 | 0.263 | 0.189 | 5.50E−41 |
| ATF7IP | 6.08E−45 | −0.346 | 0.176 | 0.122 | 6.93E−41 |
| OGFRL1 | 6.14E−45 | −0.690 | 0.122 | 0.169 | 7.00E−41 |
| GPC3 | 7.46E−45 | −0.929 | 0.012 | 0.158 | 8.50E−41 |
| DNASE2 | 8.18E−45 | −0.274 | 0.242 | 0.176 | 9.32E−41 |
| TMEM97 | 8.38E−45 | −0.362 | 0.142 | 0.105 | 9.55E−41 |
| MT-RNR1 | 9.97E−45 | −0.393 | 1 | 0.973 | 1.14E−40 |
| PCF11 | 1.19E−44 | −0.405 | 0.181 | 0.147 | 1.35E−40 |
| METTL1 | 1.49E−44 | −0.583 | 0.122 | 0.111 | 1.70E−40 |
| HIF1A | 1.68E−44 | −0.788 | 0.023 | 0.186 | 1.92E−40 |
| KXD1 | 1.71E−44 | −0.373 | 0.185 | 0.153 | 1.95E−40 |
| NUTF2 | 1.90E−44 | −0.295 | 0.211 | 0.149 | 2.16E−40 |
| H1F0 | 2.03E−44 | −0.883 | 0.154 | 0.259 | 2.31E−40 |
| ITPR2 | 2.39E−44 | −0.384 | 0.235 | 0.163 | 2.72E−40 |
| TUG1 | 2.76E−44 | −0.549 | 0.178 | 0.211 | 3.15E−40 |
| PACSIN2 | 3.08E−44 | −0.320 | 0.187 | 0.135 | 3.51E−40 |
| KPNA1 | 3.33E−44 | −0.357 | 0.163 | 0.124 | 3.79E−40 |
| ITGB8 | 3.60E−44 | −0.298 | 0.28 | 0.161 | 4.10E−40 |
| CCDC167 | 4.44E−44 | −0.336 | 0.203 | 0.146 | 5.06E−40 |
| FBXO32 | 5.76E−44 | −0.667 | 0.212 | 0.176 | 6.56E−40 |
| UBR5 | 5.76E−44 | −0.533 | 0.175 | 0.181 | 6.57E−40 |
| DTX3L | 5.98E−44 | −0.264 | 0.166 | 0.091 | 6.82E−40 |
| CENPF | 6.43E−44 | −1.532 | 0.162 | 0.318 | 7.32E−40 |
| KEAP1 | 6.88E−44 | −0.255 | 0.191 | 0.12 | 7.84E−40 |
| ZC2HC1A | 7.52E−44 | −0.622 | 0.021 | 0.182 | 8.58E−40 |
| LRPAP1 | 7.54E−44 | −0.274 | 0.197 | 0.141 | 8.59E−40 |
| BANF1 | 8.95E−44 | −0.469 | 0.187 | 0.197 | 1.02E−39 |
| TSN | 9.22E−44 | −0.416 | 0.209 | 0.163 | 1.05E−39 |
| CREBBP | 1.05E−43 | −0.300 | 0.236 | 0.148 | 1.20E−39 |
| BBX | 1.21E−43 | −0.578 | 0.176 | 0.182 | 1.38E−39 |
| PURB | 1.28E−43 | −0.323 | 0.257 | 0.163 | 1.46E−39 |
| HIST1H2BK | 1.34E−43 | −0.513 | 0.219 | 0.191 | 1.52E−39 |
| NDUFAF4 | 1.37E−43 | −0.302 | 0.149 | 0.09 | 1.56E−39 |
| H2AFX | 1.59E−43 | −0.262 | 0.184 | 0.119 | 1.81E−39 |
| TIPIN | 1.66E−43 | −0.464 | 0.187 | 0.197 | 1.89E−39 |
| ANK3 | 1.72E−43 | −0.344 | 0.258 | 0.189 | 1.96E−39 |
| IFI44L | 1.81E−43 | −0.429 | 0.163 | 0.105 | 2.06E−39 |
| EPB41 | 1.82E−43 | −0.343 | 0.137 | 0.105 | 2.08E−39 |
| FMR1 | 1.99E−43 | −0.336 | 0.206 | 0.158 | 2.27E−39 |
| LTBR | 2.30E−43 | −0.379 | 0.207 | 0.145 | 2.62E−39 |
| CHMP1B | 2.38E−43 | −0.539 | 0.179 | 0.211 | 2.71E−39 |
| ATL3 | 2.41E−43 | −0.265 | 0.249 | 0.17 | 2.75E−39 |
| MRPL48 | 3.18E−43 | −0.524 | 0.161 | 0.21 | 3.62E−39 |
| PLXNB2 | 3.51E−43 | −0.294 | 0.182 | 0.124 | 4.00E−39 |
| UBXN1 | 8.22E−43 | −0.289 | 0.219 | 0.152 | 9.37E−39 |
| SLBP | 8.42E−43 | −0.584 | 0.177 | 0.192 | 9.59E−39 |
| MPLKIP | 8.87E−43 | −0.412 | 0.193 | 0.152 | 1.01E−38 |
| RP11-349A22.5 | 1.03E−42 | −0.289 | 0.202 | 0.143 | 1.17E−38 |
| PTS | 1.08E−42 | −0.323 | 0.217 | 0.152 | 1.23E−38 |
| CISD1 | 1.15E−42 | −0.491 | 0.231 | 0.214 | 1.32E−38 |
| MCMBP | 1.33E−42 | −0.286 | 0.226 | 0.151 | 1.52E−38 |
| CSE1L | 1.34E−42 | −0.321 | 0.271 | 0.205 | 1.53E−38 |
| SENP5 | 1.82E−42 | −0.360 | 0.22 | 0.168 | 2.08E−38 |
| MTHFD1 | 1.91E−42 | −0.432 | 0.12 | 0.124 | 2.18E−38 |
| CASP8AP2 | 2.00E−42 | −0.489 | 0.185 | 0.161 | 2.28E−38 |
| MYSM1 | 2.04E−42 | −0.414 | 0.195 | 0.14 | 2.32E−38 |
| LCP1 | 2.04E−42 | −0.814 | 0.152 | 0.186 | 2.33E−38 |
| ZBTB1 | 2.13E−42 | −0.264 | 0.164 | 0.1 | 2.43E−38 |
| DPY19L4 | 2.19E−42 | −0.574 | 0.131 | 0.167 | 2.49E−38 |
| RBBP8 | 2.22E−42 | −0.607 | 0.125 | 0.165 | 2.53E−38 |
| PITPNB | 2.39E−42 | −0.495 | 0.146 | 0.146 | 2.73E−38 |
| PPP4R2 | 2.63E−42 | −0.434 | 0.179 | 0.162 | 3.00E−38 |
| KARS | 2.78E−42 | −0.325 | 0.229 | 0.157 | 3.17E−38 |
| CLPTM1L | 3.27E−42 | −0.312 | 0.241 | 0.161 | 3.72E−38 |
| COA4 | 3.34E−42 | −0.560 | 0.173 | 0.219 | 3.81E−38 |
| ELAVL1 | 3.75E−42 | −0.293 | 0.2 | 0.146 | 4.27E−38 |
| DNAJC13 | 4.12E−42 | −0.271 | 0.163 | 0.085 | 4.70E−38 |
| LINC00152 | 5.07E−42 | −0.574 | 0.095 | 0.124 | 5.77E−38 |
| CTSA | 5.18E−42 | −0.266 | 0.152 | 0.092 | 5.90E−38 |
| PDS5B | 5.35E−42 | −0.377 | 0.225 | 0.173 | 6.09E−38 |
| CIAO1 | 6.53E−42 | −0.267 | 0.182 | 0.128 | 7.45E−38 |
| UTP6 | 6.70E−42 | −0.395 | 0.152 | 0.115 | 7.64E−38 |
| ANKRD42 | 6.73E−42 | −0.474 | 0.14 | 0.174 | 7.67E−38 |
| CLCN3 | 8.26E−42 | −0.276 | 0.255 | 0.177 | 9.41E−38 |
| SETD2 | 1.07E−41 | −0.493 | 0.223 | 0.199 | 1.22E−37 |
| PYGB | 1.13E−41 | −0.254 | 0.177 | 0.119 | 1.28E−37 |
| STMN1 | 1.13E−41 | −0.918 | 0.294 | 0.434 | 1.29E−37 |
| BRD3 | 1.17E−41 | −0.262 | 0.226 | 0.165 | 1.33E−37 |
| CKB | 1.19E−41 | −0.930 | 0.015 | 0.156 | 1.35E−37 |
| FOXO3 | 1.27E−41 | −0.340 | 0.118 | 0.094 | 1.45E−37 |
| MBD2 | 1.31E−41 | −0.384 | 0.161 | 0.15 | 1.50E−37 |
| PRLR | 1.33E−41 | −0.357 | 0.196 | 0.141 | 1.51E−37 |
| YWHAH | 1.41E−41 | −0.456 | 0.207 | 0.187 | 1.60E−37 |
| LRRC26 | 1.80E−41 | −0.537 | 0.136 | 0.166 | 2.06E−37 |
| FRYL | 2.15E−41 | −0.408 | 0.197 | 0.167 | 2.45E−37 |
| KIAA1143 | 2.47E−41 | −0.280 | 0.164 | 0.11 | 2.81E−37 |
| RPL39L | 4.30E−41 | −0.488 | 0.175 | 0.166 | 4.90E−37 |
| TAF11 | 5.26E−41 | −0.283 | 0.224 | 0.16 | 6.00E−37 |
| ACTR1A | 5.35E−41 | −0.275 | 0.17 | 0.133 | 6.09E−37 |
| CUL3 | 5.67E−41 | −0.365 | 0.232 | 0.172 | 6.46E−37 |
| ASF1A | 8.81E−41 | −0.357 | 0.184 | 0.144 | 1.00E−36 |
| PTRH2 | 1.04E−40 | −0.388 | 0.16 | 0.131 | 1.19E−36 |
| DIDO1 | 1.15E−40 | −0.283 | 0.195 | 0.145 | 1.31E−36 |
| PKP2 | 1.26E−40 | −0.500 | 0.002 | 0.125 | 1.44E−36 |
| SURF6 | 1.52E−40 | −0.298 | 0.193 | 0.139 | 1.73E−36 |
| NOTCH2 | 2.31E−40 | −0.379 | 0.165 | 0.132 | 2.63E−36 |
| ICMT | 2.44E−40 | −0.355 | 0.159 | 0.141 | 2.78E−36 |
| LTV1 | 2.45E−40 | −0.600 | 0.161 | 0.17 | 2.80E−36 |
| YARS | 3.10E−40 | −0.278 | 0.261 | 0.176 | 3.53E−36 |
| CHAMP1 | 3.31E−40 | −0.295 | 0.159 | 0.116 | 3.77E−36 |
| PPP1R1B | 4.35E−40 | −0.856 | 0.001 | 0.123 | 4.95E−36 |
| GNA13 | 4.45E−40 | −0.308 | 0.136 | 0.099 | 5.07E−36 |
| RNGTT | 5.06E−40 | −0.261 | 0.105 | 0.061 | 5.77E−36 |
| SMG1 | 7.13E−40 | −0.366 | 0.229 | 0.18 | 8.13E−36 |
| TMEM126B | 9.56E−40 | −0.439 | 0.164 | 0.151 | 1.09E−35 |
| ZBTB38 | 1.08E−39 | −0.343 | 0.221 | 0.151 | 1.23E−35 |
| EPC2 | 1.31E−39 | −0.275 | 0.128 | 0.083 | 1.50E−35 |
| KIAA0947 | 1.57E−39 | −0.441 | 0.217 | 0.182 | 1.79E−35 |
| SPINK8 | 2.29E−39 | −0.527 | 0.002 | 0.125 | 2.61E−35 |
| TAF2 | 2.35E−39 | −0.498 | 0.199 | 0.208 | 2.68E−35 |
| DDIT4 | 3.02E−39 | −0.600 | 0.195 | 0.202 | 3.44E−35 |
| NT5C2 | 3.78E−39 | −0.288 | 0.163 | 0.113 | 4.31E−35 |
| RPL17 | 4.51E−39 | −0.381 | 0.197 | 0.189 | 5.14E−35 |
| ALDH3B2 | 5.18E−39 | −0.477 | 0.004 | 0.132 | 5.90E−35 |
| TRIB2 | 5.71E−39 | −0.380 | 0.187 | 0.173 | 6.50E−35 |
| MRPS9 | 6.95E−39 | −0.413 | 0.11 | 0.11 | 7.92E−35 |
| POLR2G | 7.81E−39 | −0.320 | 0.19 | 0.154 | 8.90E−35 |
| SPTAN1 | 1.02E−38 | −0.329 | 0.184 | 0.132 | 1.17E−34 |
| PTPN2 | 1.10E−38 | −0.390 | 0.144 | 0.14 | 1.26E−34 |
| CDK5RAP2 | 1.21E−38 | −0.416 | 0.171 | 0.161 | 1.37E−34 |
| PMEPA1 | 1.25E−38 | −0.510 | 0.161 | 0.155 | 1.43E−34 |
| GLRX5 | 1.36E−38 | −0.366 | 0.187 | 0.162 | 1.55E−34 |
| CLDND1 | 1.77E−38 | −0.326 | 0.137 | 0.111 | 2.01E−34 |
| PFN2 | 2.02E−38 | −0.589 | 0.019 | 0.151 | 2.31E−34 |
| CDC123 | 2.21E−38 | −0.366 | 0.233 | 0.198 | 2.52E−34 |
| MBTPS1 | 2.41E−38 | −0.348 | 0.183 | 0.148 | 2.75E−34 |
| CEBPB | 2.46E−38 | −0.514 | 0.159 | 0.177 | 2.81E−34 |
| ESYT2 | 2.59E−38 | −0.470 | 0.147 | 0.147 | 2.95E−34 |
| ZNF131 | 2.97E−38 | −0.398 | 0.178 | 0.16 | 3.39E−34 |
| GTF2H1 | 3.05E−38 | −0.483 | 0.188 | 0.209 | 3.48E−34 |
| SREBF2 | 3.09E−38 | −0.308 | 0.186 | 0.143 | 3.52E−34 |
| AGPAT5 | 4.17E−38 | −0.613 | 0.105 | 0.141 | 4.75E−34 |
| SOS1 | 4.27E−38 | −0.401 | 0.167 | 0.131 | 4.87E−34 |
| H3F3C | 4.32E−38 | −0.371 | 0.158 | 0.138 | 4.93E−34 |
| VRK1 | 5.35E−38 | −0.267 | 0.166 | 0.113 | 6.10E−34 |
| SDCCAG8 | 5.47E−38 | −0.299 | 0.18 | 0.134 | 6.24E−34 |
| XRCC6BP1 | 5.58E−38 | −0.473 | 0.101 | 0.086 | 6.36E−34 |
| ATAD2 | 5.94E−38 | −0.792 | 0.188 | 0.23 | 6.77E−34 |
| SLC4A7 | 6.20E−38 | −0.596 | 0.087 | 0.123 | 7.06E−34 |
| DUSP18 | 9.54E−38 | −0.571 | 0.058 | 0.139 | 1.09E−33 |
| PRR14L | 1.20E−37 | −0.471 | 0.125 | 0.132 | 1.36E−33 |
| HIVEP1 | 1.30E−37 | −0.275 | 0.233 | 0.157 | 1.48E−33 |
| RAB11B | 1.62E−37 | −0.322 | 0.153 | 0.134 | 1.85E−33 |
| RHOBTB1 | 1.64E−37 | −0.567 | 0.005 | 0.127 | 1.87E−33 |
| UHRF2 | 1.65E−37 | −0.253 | 0.144 | 0.091 | 1.88E−33 |
| MAP3K2 | 1.73E−37 | −0.289 | 0.166 | 0.113 | 1.97E−33 |
| KCTD3 | 1.81E−37 | −0.262 | 0.176 | 0.125 | 2.06E−33 |
| YLPM1 | 1.87E−37 | −0.372 | 0.121 | 0.107 | 2.13E−33 |
| VHL | 2.05E−37 | −0.305 | 0.228 | 0.189 | 2.34E−33 |
| UCHL1 | 2.05E−37 | −0.739 | 0 | 0.11 | 2.34E−33 |
| CNOT6L | 2.06E−37 | −0.331 | 0.161 | 0.118 | 2.35E−33 |
| CUL5 | 2.12E−37 | −0.503 | 0.218 | 0.205 | 2.42E−33 |
| C2orf15 | 2.78E−37 | −0.357 | 0.161 | 0.131 | 3.16E−33 |
| PHACTR2 | 2.83E−37 | −0.685 | 0.138 | 0.178 | 3.23E−33 |
| MRPS5 | 2.90E−37 | −0.713 | 0.086 | 0.191 | 3.31E−33 |
| TGS1 | 2.93E−37 | −0.253 | 0.232 | 0.141 | 3.34E−33 |
| NNT | 2.95E−37 | −0.296 | 0.121 | 0.095 | 3.37E−33 |
| CADM1 | 3.11E−37 | −0.624 | 0.001 | 0.115 | 3.54E−33 |
| TIMMDC1 | 3.20E−37 | −0.370 | 0.194 | 0.166 | 3.65E−33 |
| ETV6 | 3.68E−37 | −0.268 | 0.166 | 0.099 | 4.20E−33 |
| ADNP | 3.85E−37 | −0.403 | 0.192 | 0.164 | 4.39E−33 |
| MLLT4 | 5.95E−37 | −0.299 | 0.216 | 0.148 | 6.78E−33 |
| DCP2 | 6.35E−37 | −0.286 | 0.193 | 0.134 | 7.23E−33 |
| MBP | 7.02E−37 | −0.707 | 0.017 | 0.145 | 8.00E−33 |
| CCDC58 | 1.34E−36 | −0.306 | 0.165 | 0.113 | 1.53E−32 |
| NCOA3 | 1.52E−36 | −0.348 | 0.16 | 0.141 | 1.73E−32 |
| TUBGCP3 | 1.55E−36 | −0.441 | 0.125 | 0.116 | 1.76E−32 |
| DLG1 | 1.73E−36 | −0.354 | 0.226 | 0.171 | 1.97E−32 |
| NOL8 | 1.86E−36 | −0.291 | 0.192 | 0.135 | 2.12E−32 |
| IST1 | 1.97E−36 | −0.281 | 0.213 | 0.154 | 2.24E−32 |
| ODF2L | 1.98E−36 | −0.654 | 0.162 | 0.212 | 2.25E−32 |
| C11orf48 | 2.03E−36 | −0.359 | 0.185 | 0.162 | 2.31E−32 |
| NDUFV2 | 2.08E−36 | −0.265 | 0.179 | 0.143 | 2.37E−32 |
| GLS | 2.11E−36 | −0.661 | 0.054 | 0.14 | 2.40E−32 |
| RTKN2 | 2.12E−36 | −0.302 | 0.217 | 0.144 | 2.41E−32 |
| CDC42EP1 | 2.51E−36 | −0.589 | 0 | 0.106 | 2.86E−32 |
| HELLS | 3.03E−36 | −0.499 | 0.151 | 0.132 | 3.46E−32 |
| ABL2 | 3.04E−36 | −0.494 | 0.099 | 0.118 | 3.47E−32 |
| PADI2 | 3.55E−36 | −0.662 | 0.031 | 0.139 | 4.05E−32 |
| FNDC3B | 3.68E−36 | −0.558 | 0.119 | 0.135 | 4.19E−32 |
| CYB5R3 | 3.91E−36 | −0.314 | 0.158 | 0.129 | 4.46E−32 |
| TOB2 | 5.29E−36 | −0.335 | 0.22 | 0.177 | 6.03E−32 |
| ZNF90 | 5.44E−36 | −0.487 | 0.162 | 0.208 | 6.21E−32 |
| EXPH5 | 7.58E−36 | −0.469 | 0.124 | 0.125 | 8.64E−32 |
| COTL1 | 8.59E−36 | −0.816 | 0.088 | 0.189 | 9.79E−32 |
| KIAA1429 | 9.59E−36 | −0.431 | 0.193 | 0.19 | 1.09E−31 |
| OSBPL1A | 1.07E−35 | −0.332 | 0.154 | 0.129 | 1.22E−31 |
| AGAP1 | 1.19E−35 | −0.327 | 0.133 | 0.132 | 1.36E−31 |
| E2F3 | 1.35E−35 | −0.649 | 0.094 | 0.166 | 1.53E−31 |
| RANBP9 | 1.79E−35 | −0.479 | 0.157 | 0.206 | 2.05E−31 |
| GOLIM4 | 1.95E−35 | −0.273 | 0.229 | 0.146 | 2.23E−31 |
| CWC22 | 2.42E−35 | −0.326 | 0.181 | 0.138 | 2.76E−31 |
| ATP11B | 2.51E−35 | −0.670 | 0.033 | 0.169 | 2.86E−31 |
| FAM111A | 2.71E−35 | −0.254 | 0.15 | 0.088 | 3.09E−31 |
| HPS3 | 2.83E−35 | −0.301 | 0.129 | 0.096 | 3.23E−31 |
| FAR1 | 2.85E−35 | −0.284 | 0.162 | 0.115 | 3.25E−31 |
| IGFBP3 | 3.00E−35 | −1.137 | 0.002 | 0.112 | 3.42E−31 |
| UCK2 | 3.08E−35 | −0.355 | 0.112 | 0.095 | 3.51E−31 |
| PITRM1 | 3.73E−35 | −0.374 | 0.216 | 0.195 | 4.25E−31 |
| LRRFIP2 | 3.80E−35 | −0.313 | 0.189 | 0.14 | 4.33E−31 |
| RGS2 | 3.83E−35 | −0.679 | 0.129 | 0.137 | 4.36E−31 |
| PTPRK | 3.91E−35 | −0.480 | 0.159 | 0.167 | 4.46E−31 |
| NDRG1 | 4.43E−35 | −1.019 | 0.071 | 0.204 | 5.05E−31 |
| NOM1 | 5.17E−35 | −0.267 | 0.125 | 0.075 | 5.89E−31 |
| TRIO | 5.55E−35 | −0.531 | 0.099 | 0.131 | 6.33E−31 |
| PHGDH | 5.63E−35 | −0.712 | 0.091 | 0.201 | 6.41E−31 |
| TEX30 | 6.21E−35 | −0.392 | 0.119 | 0.122 | 7.08E−31 |
| AIM1 | 6.95E−35 | −0.724 | 0.018 | 0.134 | 7.92E−31 |
| PVT1 | 7.75E−35 | −1.166 | 0.011 | 0.129 | 8.83E−31 |
| DTD1 | 8.33E−35 | −0.516 | 0.097 | 0.133 | 9.49E−31 |
| C3orf14 | 8.71E−35 | −0.322 | 0.197 | 0.157 | 9.92E−31 |
| NFIX | 9.69E−35 | −0.513 | 0.121 | 0.148 | 1.10E−30 |
| FUCA2 | 9.98E−35 | −0.335 | 0.15 | 0.131 | 1.14E−30 |
| WWTR1 | 1.04E−34 | −0.750 | 0.027 | 0.135 | 1.18E−30 |
| PRNP | 1.09E−34 | −0.716 | 0.017 | 0.151 | 1.24E−30 |
| ROCK2 | 1.15E−34 | −0.701 | 0.123 | 0.189 | 1.31E−30 |
| WDR70 | 1.38E−34 | −0.407 | 0.172 | 0.155 | 1.57E−30 |
| PDCD11 | 1.63E−34 | −0.426 | 0.123 | 0.127 | 1.86E−30 |
| AHCTF1 | 1.68E−34 | −0.447 | 0.135 | 0.13 | 1.92E−30 |
| SNX4 | 1.69E−34 | −0.274 | 0.161 | 0.121 | 1.93E−30 |
| CHMP4C | 1.74E−34 | −0.363 | 0.22 | 0.182 | 1.99E−30 |
| REXO2 | 1.93E−34 | −0.408 | 0.148 | 0.144 | 2.20E−30 |
| TMA16 | 2.03E−34 | −0.252 | 0.22 | 0.154 | 2.31E−30 |
| GPR180 | 2.12E−34 | −0.408 | 0.128 | 0.111 | 2.42E−30 |
| TEX10 | 2.28E−34 | −0.498 | 0.119 | 0.135 | 2.60E−30 |
| HSF1 | 2.44E−34 | −0.267 | 0.237 | 0.17 | 2.78E−30 |
| C19orf48 | 2.49E−34 | −0.579 | 0.107 | 0.176 | 2.84E−30 |
| CASC7 | 3.40E−34 | −0.542 | 0.118 | 0.126 | 3.88E−30 |
| SNHG3 | 3.74E−34 | −0.621 | 0.089 | 0.195 | 4.26E−30 |
| CCDC90B | 4.14E−34 | −0.509 | 0.142 | 0.183 | 4.72E−30 |
| PTRH1 | 5.58E−34 | −0.346 | 0.137 | 0.094 | 6.35E−30 |
| CEP95 | 6.35E−34 | −0.486 | 0.138 | 0.166 | 7.24E−30 |
| PGAP3 | 6.63E−34 | −0.518 | 0.056 | 0.154 | 7.56E−30 |
| HLTF | 9.04E−34 | −0.463 | 0.17 | 0.166 | 1.03E−29 |
| TGIF1 | 1.04E−33 | −0.256 | 0.22 | 0.151 | 1.19E−29 |
| FAM192A | 1.20E−33 | −0.370 | 0.15 | 0.138 | 1.37E−29 |
| GNB4 | 1.31E−33 | −0.605 | 0.006 | 0.116 | 1.49E−29 |
| ANKRD36 | 1.34E−33 | −0.826 | 0.087 | 0.161 | 1.52E−29 |
| TOMM70A | 1.39E−33 | −0.322 | 0.195 | 0.157 | 1.58E−29 |
| SOCS4 | 1.40E−33 | −0.318 | 0.149 | 0.115 | 1.59E−29 |
| RIOK1 | 1.45E−33 | −0.310 | 0.148 | 0.115 | 1.65E−29 |
| NME1-NME2 | 1.74E−33 | −0.432 | 0.14 | 0.17 | 1.98E−29 |
| SEC24C | 1.97E−33 | −0.312 | 0.143 | 0.141 | 2.25E−29 |
| ATP6V1C1 | 2.10E−33 | −0.384 | 0.16 | 0.141 | 2.39E−29 |
| PHKB | 2.11E−33 | −0.371 | 0.144 | 0.142 | 2.41E−29 |
| UGGT2 | 2.16E−33 | −0.316 | 0.153 | 0.111 | 2.47E−29 |
| QKI | 3.13E−33 | −0.651 | 0.043 | 0.132 | 3.57E−29 |
| DNAH14 | 3.35E−33 | −0.520 | 0.133 | 0.149 | 3.82E−29 |
| SGK1 | 3.37E−33 | −0.858 | 0.071 | 0.134 | 3.85E−29 |
| UBE2M | 3.60E−33 | −0.322 | 0.161 | 0.142 | 4.10E−29 |
| RUVBL2 | 3.64E−33 | −0.255 | 0.198 | 0.146 | 4.15E−29 |
| COPRS | 4.32E−33 | −0.256 | 0.122 | 0.075 | 4.92E−29 |
| ASUN | 5.15E−33 | −0.375 | 0.141 | 0.127 | 5.87E−29 |
| ZMIZ1 | 5.79E−33 | −0.282 | 0.249 | 0.193 | 6.60E−29 |
| BTG3 | 6.04E−33 | −0.855 | 0.047 | 0.161 | 6.89E−29 |
| TRIM28 | 6.38E−33 | −0.456 | 0.116 | 0.163 | 7.28E−29 |
| ZMYND8 | 6.48E−33 | −0.424 | 0.19 | 0.181 | 7.38E−29 |
| EPB41L2 | 6.56E−33 | −0.526 | 0.08 | 0.103 | 7.48E−29 |
| PRRC2A | 6.96E−33 | −0.482 | 0.101 | 0.141 | 7.93E−29 |
| GNL3L | 7.33E−33 | −0.260 | 0.12 | 0.082 | 8.35E−29 |
| CFDP1 | 8.48E−33 | −0.467 | 0.132 | 0.14 | 9.67E−29 |
| EMG1 | 8.58E−33 | −0.300 | 0.166 | 0.139 | 9.78E−29 |
| USP36 | 8.82E−33 | −0.296 | 0.161 | 0.122 | 1.01E−28 |
| ARRDC3 | 9.56E−33 | −0.588 | 0.016 | 0.127 | 1.09E−28 |
| GANAB | 1.08E−32 | −0.603 | 0.035 | 0.162 | 1.23E−28 |
| WDR3 | 1.26E−32 | −0.469 | 0.124 | 0.137 | 1.43E−28 |
| LYZ | 1.28E−32 | −0.351 | 0.196 | 0.116 | 1.45E−28 |
| INTS8 | 1.45E−32 | −0.332 | 0.153 | 0.137 | 1.65E−28 |
| NAE1 | 1.49E−32 | −0.282 | 0.179 | 0.141 | 1.70E−28 |
| C8orf4 | 2.04E−32 | −0.853 | 0.013 | 0.119 | 2.32E−28 |
| CLIC4 | 2.33E−32 | −0.398 | 0.198 | 0.192 | 2.65E−28 |
| PRPF19 | 2.54E−32 | −0.276 | 0.163 | 0.132 | 2.89E−28 |
| PCYOX1 | 2.68E−32 | −0.474 | 0.164 | 0.189 | 3.05E−28 |
| GADD45A | 3.57E−32 | −0.285 | 0.166 | 0.095 | 4.07E−28 |
| TSPAN1 | 3.67E−32 | −0.414 | 0.001 | 0.101 | 4.18E−28 |
| CWF19L2 | 4.83E−32 | −0.636 | 0.102 | 0.151 | 5.50E−28 |
| TMEM254 | 5.01E−32 | −0.374 | 0.175 | 0.174 | 5.71E−28 |
| LRP6 | 5.55E−32 | −0.286 | 0.161 | 0.122 | 6.33E−28 |
| PMP22 | 5.86E−32 | −0.651 | 0.096 | 0.159 | 6.68E−28 |
| HLA-DRA | 5.99E−32 | −0.808 | 0.144 | 0.17 | 6.82E−28 |
| PLAGL1 | 7.48E−32 | −0.554 | 0.003 | 0.105 | 8.52E−28 |
| TMED1 | 7.58E−32 | −0.253 | 0.118 | 0.087 | 8.64E−28 |
| STK25 | 7.60E−32 | −0.294 | 0.16 | 0.127 | 8.66E−28 |
| PTCD3 | 7.83E−32 | −0.419 | 0.147 | 0.138 | 8.92E−28 |
| CPQ | 8.11E−32 | −0.602 | 0.018 | 0.113 | 9.24E−28 |
| ITGA6 | 8.61E−32 | −0.669 | 0.033 | 0.133 | 9.82E−28 |
| CHMP1A | 8.65E−32 | −0.358 | 0.141 | 0.125 | 9.86E−28 |
| BRD9 | 9.89E−32 | −0.329 | 0.11 | 0.087 | 1.13E−27 |
| PDRG1 | 1.11E−31 | −0.260 | 0.148 | 0.127 | 1.27E−27 |
| CTSC | 1.13E−31 | −0.661 | 0.036 | 0.138 | 1.29E−27 |
| IRS2 | 1.19E−31 | −0.727 | 0.079 | 0.145 | 1.36E−27 |
| PHLDA1 | 1.20E−31 | −0.433 | 0.139 | 0.115 | 1.37E−27 |
| RPL36A | 1.38E−31 | −0.340 | 0.139 | 0.132 | 1.58E−27 |
| MTR | 1.60E−31 | −0.316 | 0.124 | 0.102 | 1.82E−27 |
| CCNE2 | 1.74E−31 | −0.710 | 0.127 | 0.186 | 1.98E−27 |
| NR2C2AP | 1.76E−31 | −0.256 | 0.144 | 0.108 | 2.01E−27 |
| PPAT | 1.96E−31 | −0.585 | 0.088 | 0.135 | 2.24E−27 |
| IRF2BPL | 3.14E−31 | −0.302 | 0.122 | 0.11 | 3.58E−27 |
| RRP7A | 3.23E−31 | −0.362 | 0.125 | 0.138 | 3.69E−27 |
| AQP5 | 3.58E−31 | −0.481 | 0.003 | 0.105 | 4.08E−27 |
| CCDC66 | 4.29E−31 | −0.261 | 0.126 | 0.103 | 4.89E−27 |
| CCDC14 | 4.53E−31 | −0.737 | 0.087 | 0.148 | 5.16E−27 |
| CLCA2 | 4.81E−31 | −0.600 | 0.003 | 0.106 | 5.49E−27 |
| PIK3CA | 6.53E−31 | −0.365 | 0.125 | 0.108 | 7.44E−27 |
| MYO9B | 7.26E−31 | −0.412 | 0.082 | 0.103 | 8.28E−27 |
| SLC2A4RG | 8.00E−31 | −0.267 | 0.158 | 0.13 | 9.12E−27 |
| MGAT4A | 8.04E−31 | −0.279 | 0.142 | 0.107 | 9.16E−27 |
| NOL11 | 8.99E−31 | −0.368 | 0.128 | 0.11 | 1.02E−26 |
| DNMT3A | 9.96E−31 | −0.286 | 0.154 | 0.121 | 1.13E−26 |
| PTDSS1 | 1.28E−30 | −0.489 | 0.089 | 0.138 | 1.46E−26 |
| AUTS2 | 1.76E−30 | −0.397 | 0.114 | 0.118 | 2.01E−26 |
| CD52 | 1.87E−30 | −0.682 | 0.125 | 0.111 | 2.13E−26 |
| MED30 | 2.32E−30 | −0.274 | 0.131 | 0.112 | 2.64E−26 |
| H1FX | 2.56E−30 | −0.341 | 0.15 | 0.148 | 2.91E−26 |
| SMC6 | 2.69E−30 | −0.566 | 0.137 | 0.149 | 3.07E−26 |
| COA5 | 3.12E−30 | −0.441 | 0.144 | 0.151 | 3.56E−26 |
| TCF3 | 3.61E−30 | −0.408 | 0.094 | 0.11 | 4.11E−26 |
| NDRG2 | 3.89E−30 | −0.430 | 0.103 | 0.091 | 4.43E−26 |
| SAMD12 | 4.29E−30 | −0.265 | 0.158 | 0.114 | 4.89E−26 |
| SERPINH1 | 4.39E−30 | −0.487 | 0.113 | 0.169 | 5.00E−26 |
| IFI16 | 4.47E−30 | −0.885 | 0.026 | 0.106 | 5.10E−26 |
| EXOC3 | 4.70E−30 | −0.298 | 0.125 | 0.104 | 5.35E−26 |
| SLC16A1 | 5.27E−30 | −0.567 | 0.015 | 0.115 | 6.01E−26 |
| FTSJ3 | 5.28E−30 | −0.304 | 0.141 | 0.11 | 6.01E−26 |
| WDR12 | 5.91E−30 | −0.362 | 0.1 | 0.106 | 6.73E−26 |
| KIN | 5.99E−30 | −0.343 | 0.162 | 0.144 | 6.83E−26 |
| GSK3B | 6.63E−30 | −0.341 | 0.121 | 0.12 | 7.56E−26 |
| UPF3A | 7.87E−30 | −0.359 | 0.122 | 0.125 | 8.97E−26 |
| BIRC5 | 8.37E−30 | −0.867 | 0.145 | 0.205 | 9.55E−26 |
| POLE4 | 8.55E−30 | −0.256 | 0.122 | 0.094 | 9.75E−26 |
| NUP85 | 8.88E−30 | −0.273 | 0.14 | 0.126 | 1.01E−25 |
| ASXL2 | 9.25E−30 | −0.288 | 0.148 | 0.103 | 1.05E−25 |
| TNPO2 | 1.02E−29 | −0.507 | 0.071 | 0.168 | 1.16E−25 |
| PDP1 | 1.22E−29 | −0.418 | 0.165 | 0.169 | 1.39E−25 |
| SLFN13 | 1.33E−29 | −0.493 | 0.004 | 0.106 | 1.51E−25 |
| NLN | 1.75E−29 | −0.450 | 0.125 | 0.134 | 1.99E−25 |
| ZCCHC11 | 1.90E−29 | −0.681 | 0.059 | 0.154 | 2.16E−25 |
| OTUD6B | 2.24E−29 | −0.465 | 0.063 | 0.103 | 2.55E−25 |
| XPNPEP3 | 2.47E−29 | −0.337 | 0.119 | 0.121 | 2.82E−25 |
| RCC2 | 2.55E−29 | −0.296 | 0.154 | 0.131 | 2.90E−25 |
| LINC00998 | 2.73E−29 | −0.599 | 0.112 | 0.18 | 3.11E−25 |
| TET2 | 3.22E−29 | −0.252 | 0.144 | 0.072 | 3.67E−25 |
| ABHD2 | 3.71E−29 | −0.289 | 0.116 | 0.089 | 4.23E−25 |
| NARF | 4.41E−29 | −0.481 | 0.14 | 0.175 | 5.03E−25 |
| CCDC88A | 4.43E−29 | −0.770 | 0.014 | 0.113 | 5.05E−25 |
| PSIP1 | 4.50E−29 | −0.476 | 0.145 | 0.165 | 5.13E−25 |
| INSIG2 | 5.29E−29 | −0.398 | 0.087 | 0.109 | 6.02E−25 |
| SEH1L | 5.95E−29 | −0.473 | 0.077 | 0.149 | 6.79E−25 |
| ZNF791 | 6.57E−29 | −0.320 | 0.175 | 0.152 | 7.48E−25 |
| RUFY2 | 9.40E−29 | −0.283 | 0.156 | 0.116 | 1.07E−24 |
| CMSS1 | 9.63E−29 | −0.507 | 0.175 | 0.176 | 1.10E−24 |
| LAPTM4B | 1.06E−28 | −0.611 | 0.029 | 0.149 | 1.21E−24 |
| WDR75 | 1.07E−28 | −0.359 | 0.144 | 0.128 | 1.22E−24 |
| NPM3 | 1.12E−28 | −0.470 | 0.072 | 0.148 | 1.28E−24 |
| AP3M1 | 1.13E−28 | −0.254 | 0.161 | 0.147 | 1.29E−24 |
| SEC14L1 | 1.19E−28 | −0.252 | 0.12 | 0.101 | 1.35E−24 |
| SLC25A1 | 1.33E−28 | −0.275 | 0.165 | 0.148 | 1.52E−24 |
| LRRC58 | 1.47E−28 | −0.415 | 0.107 | 0.144 | 1.67E−24 |
| CCDC50 | 1.52E−28 | −0.627 | 0.063 | 0.167 | 1.74E−24 |
| RPSAP58 | 1.56E−28 | −0.403 | 0.083 | 0.128 | 1.78E−24 |
| NOC3L | 1.76E−28 | −0.444 | 0.145 | 0.131 | 2.00E−24 |
| CSNK1E | 1.80E−28 | −0.375 | 0.12 | 0.139 | 2.05E−24 |
| OTUB1 | 2.26E−28 | −0.251 | 0.151 | 0.12 | 2.57E−24 |
| MAGEF1 | 2.36E−28 | −0.420 | 0.11 | 0.134 | 2.69E−24 |
| AP1B1 | 2.42E−28 | −0.291 | 0.123 | 0.11 | 2.76E−24 |
| PHF5A | 2.55E−28 | −0.275 | 0.156 | 0.141 | 2.91E−24 |
| NANOS1 | 2.63E−28 | −0.555 | 0.01 | 0.106 | 3.00E−24 |
| AFMID | 2.63E−28 | −0.415 | 0.071 | 0.125 | 3.00E−24 |
| MTAP | 3.12E−28 | −0.370 | 0.125 | 0.113 | 3.56E−24 |
| CTD-2090I13.1 | 3.63E−28 | −0.457 | 0.087 | 0.129 | 4.14E−24 |
| NDC1 | 4.14E−28 | −0.266 | 0.107 | 0.088 | 4.72E−24 |
| SMARCA1 | 4.93E−28 | −0.547 | 0.004 | 0.101 | 5.62E−24 |
| UPF3B | 5.01E−28 | −0.288 | 0.158 | 0.109 | 5.71E−24 |
| HERC4 | 5.05E−28 | −0.376 | 0.132 | 0.14 | 5.76E−24 |
| DDX10 | 5.16E−28 | −0.484 | 0.076 | 0.122 | 5.89E−24 |
| HOOK2 | 5.88E−28 | −0.407 | 0.117 | 0.142 | 6.70E−24 |
| AGL | 5.95E−28 | −0.364 | 0.129 | 0.127 | 6.78E−24 |
| AASDHPPT | 6.29E−28 | −0.393 | 0.159 | 0.151 | 7.17E−24 |
| MSN | 6.31E−28 | −0.676 | 0.027 | 0.103 | 7.19E−24 |
| FKBP5 | 6.75E−28 | −0.543 | 0.03 | 0.108 | 7.70E−24 |
| ZZZ3 | 8.21E−28 | −0.387 | 0.131 | 0.135 | 9.35E−24 |
| LYAR | 1.48E−27 | −0.493 | 0.087 | 0.118 | 1.69E−23 |
| RUVBL1 | 2.04E−27 | −0.451 | 0.123 | 0.124 | 2.32E−23 |
| AP1S2 | 3.16E−27 | −0.306 | 0.105 | 0.079 | 3.60E−23 |
| NIN | 3.32E−27 | −0.253 | 0.14 | 0.11 | 3.79E−23 |
| ALYREF | 3.45E−27 | −0.327 | 0.114 | 0.112 | 3.93E−23 |
| ANLN | 4.16E−27 | −0.606 | 0.17 | 0.186 | 4.74E−23 |
| EP300 | 4.38E−27 | −0.423 | 0.132 | 0.146 | 4.99E−23 |
| THAP9-AS1 | 4.59E−27 | −0.315 | 0.109 | 0.091 | 5.23E−23 |
| FBXL20 | 5.37E−27 | −0.348 | 0.018 | 0.111 | 6.12E−23 |
| NEDD9 | 5.47E−27 | −0.622 | 0.089 | 0.135 | 6.23E−23 |
| RRS1 | 5.61E−27 | −0.318 | 0.104 | 0.098 | 6.40E−23 |
| LYRM4 | 8.32E−27 | −0.280 | 0.139 | 0.132 | 9.49E−23 |
| RAB12 | 8.74E−27 | −0.422 | 0.097 | 0.123 | 9.96E−23 |
| VPS13A | 9.80E−27 | −0.471 | 0.104 | 0.103 | 1.12E−22 |
| COLGALT1 | 1.23E−26 | −0.450 | 0.077 | 0.128 | 1.41E−22 |
| ZNHIT6 | 1.45E−26 | −0.284 | 0.148 | 0.12 | 1.65E−22 |
| UBXN7 | 1.51E−26 | −0.389 | 0.109 | 0.116 | 1.72E−22 |
| RBM6 | 1.94E−26 | −0.346 | 0.112 | 0.106 | 2.21E−22 |
| EXOSC5 | 2.06E−26 | −0.289 | 0.125 | 0.12 | 2.34E−22 |
| SLC35F2 | 2.35E−26 | −0.548 | 0.006 | 0.102 | 2.68E−22 |
| SMURF2 | 2.77E−26 | −0.667 | 0.056 | 0.132 | 3.16E−22 |
| UBP1 | 2.88E−26 | −0.289 | 0.105 | 0.097 | 3.28E−22 |
| EFHD1 | 3.73E−26 | −0.480 | 0.047 | 0.163 | 4.25E−22 |
| ATP2B1 | 5.61E−26 | −0.507 | 0.061 | 0.113 | 6.39E−22 |
| PHF6 | 5.63E−26 | −0.349 | 0.103 | 0.115 | 6.42E−22 |
| SMARCB1 | 6.41E−26 | −0.299 | 0.141 | 0.135 | 7.31E−22 |
| NOP14 | 6.46E−26 | −0.371 | 0.119 | 0.125 | 7.36E−22 |
| FRMD6 | 7.98E−26 | −0.549 | 0.092 | 0.122 | 9.09E−22 |
| GMNN | 8.24E−26 | −0.584 | 0.131 | 0.189 | 9.39E−22 |
| CCDC117 | 8.88E−26 | −0.374 | 0.083 | 0.11 | 1.01E−21 |
| RNMT | 9.63E−26 | −0.490 | 0.138 | 0.179 | 1.10E−21 |
| ZNF22 | 1.06E−25 | −0.319 | 0.142 | 0.145 | 1.21E−21 |
| TBC1D7 | 1.08E−25 | −0.281 | 0.117 | 0.125 | 1.23E−21 |
| CREBZF | 1.14E−25 | −0.377 | 0.089 | 0.108 | 1.30E−21 |
| HCFC1 | 1.18E−25 | −0.258 | 0.128 | 0.1 | 1.34E−21 |
| CHERP | 1.19E−25 | −0.279 | 0.106 | 0.089 | 1.36E−21 |
| ARHGEF7 | 1.29E−25 | −0.340 | 0.114 | 0.106 | 1.47E−21 |
| BICD2 | 1.36E−25 | −0.259 | 0.114 | 0.078 | 1.56E−21 |
| SRGN | 1.40E−25 | −1.166 | 0.075 | 0.179 | 1.60E−21 |
| MMGT1 | 1.42E−25 | −0.290 | 0.114 | 0.101 | 1.61E−21 |
| CCDC137 | 1.73E−25 | −0.335 | 0.152 | 0.129 | 1.97E−21 |
| CORO1C | 1.81E−25 | −0.324 | 0.106 | 0.09 | 2.07E−21 |
| TAF1B | 2.01E−25 | −0.375 | 0.14 | 0.125 | 2.29E−21 |
| RUFY3 | 2.09E−25 | −0.321 | 0.123 | 0.109 | 2.38E−21 |
| AKAP1 | 2.12E−25 | −0.332 | 0.114 | 0.118 | 2.41E−21 |
| SLC40A1 | 2.55E−25 | −0.501 | 0.12 | 0.158 | 2.91E−21 |
| ZFC3H1 | 3.52E−25 | −0.257 | 0.129 | 0.091 | 4.02E−21 |
| ADD1 | 3.53E−25 | −0.273 | 0.152 | 0.119 | 4.02E−21 |
| ING1 | 3.82E−25 | −0.382 | 0.101 | 0.059 | 4.35E−21 |
| MRPL16 | 3.82E−25 | −0.301 | 0.121 | 0.102 | 4.35E−21 |
| APOE | 5.02E−25 | −0.667 | 0.022 | 0.134 | 5.72E−21 |
| KCNMA1 | 5.11E−25 | −0.578 | 0.04 | 0.135 | 5.82E−21 |
| PICALM | 6.09E−25 | −0.501 | 0.1 | 0.178 | 6.95E−21 |
| SHPRH | 7.63E−25 | −0.486 | 0.082 | 0.106 | 8.70E−21 |
| PTPLB | 8.54E−25 | −0.454 | 0.165 | 0.191 | 9.73E−21 |
| CEP57 | 8.87E−25 | −0.532 | 0.095 | 0.167 | 1.01E−20 |
| DHTKD1 | 9.24E−25 | −0.266 | 0.171 | 0.148 | 1.05E−20 |
| ETS1 | 1.19E−24 | −0.795 | 0.054 | 0.135 | 1.35E−20 |
| HIST1H2AC | 1.28E−24 | −0.395 | 0.105 | 0.116 | 1.46E−20 |
| VCPIP1 | 1.30E−24 | −0.264 | 0.116 | 0.087 | 1.49E−20 |
| MRTO4 | 1.48E−24 | −0.305 | 0.117 | 0.123 | 1.69E−20 |
| IMPACT | 1.50E−24 | −0.255 | 0.135 | 0.107 | 1.71E−20 |
| CRIM1 | 2.64E−24 | −0.596 | 0.041 | 0.113 | 3.00E−20 |
| MDN1 | 3.61E−24 | −0.563 | 0.062 | 0.119 | 4.12E−20 |
| KIF1B | 3.66E−24 | −0.387 | 0.105 | 0.112 | 4.17E−20 |
| MOB1B | 4.69E−24 | −0.421 | 0.102 | 0.109 | 5.34E−20 |
| DCUN1D4 | 4.72E−24 | −0.440 | 0.075 | 0.113 | 5.38E−20 |
| KLHL23 | 5.29E−24 | −0.551 | 0.025 | 0.108 | 6.03E−20 |
| PRKRIR | 5.35E−24 | −0.376 | 0.036 | 0.112 | 6.10E−20 |
| SLC25A32 | 6.47E−24 | −0.297 | 0.102 | 0.105 | 7.38E−20 |
| TCEB3B | 7.22E−24 | −0.612 | 0.047 | 0.112 | 8.22E−20 |
| RC3H2 | 7.55E−24 | −0.396 | 0.102 | 0.125 | 8.61E−20 |
| CCDC23 | 7.61E−24 | −0.274 | 0.123 | 0.11 | 8.67E−20 |
| ZC3H18 | 8.75E−24 | −0.270 | 0.102 | 0.084 | 9.98E−20 |
| WRN | 8.77E−24 | −0.356 | 0.101 | 0.119 | 9.99E−20 |
| LRIG1 | 9.10E−24 | −0.304 | 0.087 | 0.106 | 1.04E−19 |
| ZNF260 | 1.02E−23 | −0.345 | 0.108 | 0.118 | 1.16E−19 |
| WDR82 | 1.04E−23 | −0.325 | 0.093 | 0.106 | 1.19E−19 |
| MKI67 | 1.23E−23 | −0.856 | 0.131 | 0.183 | 1.40E−19 |
| AIG1 | 1.39E−23 | −0.429 | 0.062 | 0.149 | 1.58E−19 |
| CTSZ | 1.60E−23 | −0.500 | 0.063 | 0.118 | 1.82E−19 |
| FZD6 | 1.91E−23 | −0.272 | 0.126 | 0.111 | 2.18E−19 |
| HIST1H1E | 2.99E−23 | −0.472 | 0.11 | 0.145 | 3.41E−19 |
| ELP5 | 3.21E−23 | −0.351 | 0.106 | 0.125 | 3.65E−19 |
| RNF145 | 3.27E−23 | −0.474 | 0.012 | 0.102 | 3.72E−19 |
| KDSR | 3.44E−23 | −0.336 | 0.152 | 0.16 | 3.92E−19 |
| CD3EAP | 4.44E−23 | −0.411 | 0.094 | 0.121 | 5.07E−19 |
| ARID5B | 4.83E−23 | −0.516 | 0.102 | 0.176 | 5.50E−19 |
| FAM210A | 5.14E−23 | −0.379 | 0.074 | 0.133 | 5.86E−19 |
| THOC1 | 5.18E−23 | −0.397 | 0.106 | 0.117 | 5.91E−19 |
| CTD-2192J16.22 | 6.92E−23 | −0.390 | 0.108 | 0.172 | 7.89E−19 |
| MAPK1 | 7.35E−23 | −0.331 | 0.138 | 0.144 | 8.37E−19 |
| SIRT5 | 7.50E−23 | −0.379 | 0.072 | 0.125 | 8.55E−19 |
| PSMG1 | 1.14E−22 | −0.439 | 0.081 | 0.133 | 1.30E−18 |
| WHSC1 | 1.38E−22 | −0.475 | 0.099 | 0.14 | 1.57E−18 |
| POLD3 | 1.76E−22 | −0.363 | 0.089 | 0.136 | 2.01E−18 |
| SMC2 | 2.29E−22 | −0.377 | 0.178 | 0.16 | 2.61E−18 |
| ZCCHC2 | 2.48E−22 | −0.331 | 0.099 | 0.103 | 2.83E−18 |
| DLAT | 3.65E−22 | −0.361 | 0.091 | 0.107 | 4.16E−18 |
| PPP4R1 | 4.63E−22 | −0.347 | 0.107 | 0.135 | 5.28E−18 |
| HNRNPUL2 | 4.68E−22 | −0.309 | 0.116 | 0.116 | 5.34E−18 |
| ENPP1 | 4.76E−22 | −0.410 | 0.101 | 0.151 | 5.43E−18 |
| HIST1H4H | 4.86E−22 | −0.542 | 0.032 | 0.125 | 5.54E−18 |
| INTS10 | 6.22E−22 | −0.305 | 0.107 | 0.103 | 7.09E−18 |
| RIMS2 | 6.61E−22 | −0.399 | 0.055 | 0.106 | 7.54E−18 |
| C11orf54 | 6.92E−22 | −0.372 | 0.107 | 0.118 | 7.88E−18 |
| NCBP2-AS2 | 7.50E−22 | −0.356 | 0.123 | 0.133 | 8.55E−18 |
| CSPP1 | 9.05E−22 | −0.490 | 0.12 | 0.129 | 1.03E−17 |
| SAMD8 | 1.17E−21 | −0.275 | 0.138 | 0.144 | 1.33E−17 |
| ST6GAL1 | 1.29E−21 | −0.405 | 0.033 | 0.124 | 1.46E−17 |
| CD109 | 1.39E−21 | −0.569 | 0.028 | 0.102 | 1.58E−17 |
| RP11-58E21.4 | 1.47E−21 | −0.356 | 0.067 | 0.11 | 1.67E−17 |
| UXS1 | 2.12E−21 | −0.344 | 0.111 | 0.115 | 2.42E−17 |
| RAB30-AS1 | 2.45E−21 | −0.390 | 0.059 | 0.141 | 2.80E−17 |
| PDCL3 | 2.93E−21 | −0.290 | 0.117 | 0.11 | 3.34E−17 |
| ARL4C | 3.00E−21 | −0.503 | 0.02 | 0.104 | 3.41E−17 |
| RBM5 | 3.08E−21 | −0.299 | 0.114 | 0.102 | 3.51E−17 |
| IMPA1 | 3.49E−21 | −0.294 | 0.122 | 0.108 | 3.98E−17 |
| CEP135 | 4.16E−21 | −0.264 | 0.115 | 0.086 | 4.75E−17 |
| BCL6 | 8.55E−21 | −0.437 | 0.051 | 0.119 | 9.74E−17 |
| KLHL24 | 1.36E−20 | −0.320 | 0.092 | 0.102 | 1.55E−16 |
| NFXL1 | 1.38E−20 | −0.284 | 0.088 | 0.105 | 1.58E−16 |
| C11orf73 | 2.80E−20 | −0.399 | 0.117 | 0.148 | 3.19E−16 |
| ACAT1 | 2.81E−20 | −0.468 | 0.063 | 0.141 | 3.20E−16 |
| PRSS23 | 2.91E−20 | −0.434 | 0.031 | 0.121 | 3.32E−16 |
| SLC5A3 | 2.98E−20 | −0.439 | 0.053 | 0.105 | 3.39E−16 |
| KIF20B | 3.68E−20 | −0.355 | 0.189 | 0.159 | 4.19E−16 |
| GCDH | 3.81E−20 | −0.337 | 0.082 | 0.116 | 4.34E−16 |
| C11orf57 | 4.09E−20 | −0.389 | 0.105 | 0.125 | 4.66E−16 |
| PTPRC | 4.68E−20 | −0.452 | 0.106 | 0.097 | 5.33E−16 |
| HIST1H1D | 5.55E−20 | −0.459 | 0.145 | 0.135 | 6.32E−16 |
| USE1 | 1.78E−19 | −0.321 | 0.093 | 0.106 | 2.03E−15 |
| AARS | 2.22E−19 | −0.265 | 0.099 | 0.11 | 2.53E−15 |
| ZFYVE20 | 2.38E−19 | −0.252 | 0.113 | 0.105 | 2.71E−15 |
| PCMTD2 | 2.42E−19 | −0.292 | 0.092 | 0.103 | 2.76E−15 |
| MAP2K7 | 2.72E−19 | −0.283 | 0.116 | 0.111 | 3.10E−15 |
| NACC1 | 4.08E−19 | −0.271 | 0.103 | 0.133 | 4.65E−15 |
| LINC00665 | 5.26E−19 | −0.384 | 0.058 | 0.11 | 6.00E−15 |
| MTERFD1 | 6.08E−19 | −0.266 | 0.071 | 0.103 | 6.93E−15 |
| LIMK2 | 8.21E−19 | −0.251 | 0.098 | 0.106 | 9.36E−15 |
| HMGB2 | 1.17E−18 | −0.605 | 0.279 | 0.329 | 1.33E−14 |
| C8orf82 | 1.26E−18 | −0.254 | 0.079 | 0.117 | 1.44E−14 |
| ERC1 | 2.45E−18 | −0.270 | 0.109 | 0.112 | 2.79E−14 |
| PES1 | 2.55E−18 | −0.351 | 0.07 | 0.106 | 2.90E−14 |
| TPX2 | 2.59E−18 | −0.650 | 0.135 | 0.179 | 2.95E−14 |
| SAPCD2 | 3.98E−18 | −0.312 | 0.066 | 0.108 | 4.54E−14 |
| G6PD | 5.86E−18 | −0.262 | 0.12 | 0.116 | 6.68E−14 |
| TRIM37 | 9.68E−18 | −0.253 | 0.113 | 0.103 | 1.10E−13 |
| TIMM21 | 1.41E−17 | −0.293 | 0.066 | 0.102 | 1.61E−13 |
| CCDC34 | 1.50E−17 | −0.321 | 0.118 | 0.13 | 1.71E−13 |
| CHORDC1 | 1.59E−17 | −0.392 | 0.087 | 0.132 | 1.81E−13 |
| TOMM40 | 1.60E−17 | −0.277 | 0.085 | 0.116 | 1.82E−13 |
| SLC39A4 | 2.74E−17 | −0.371 | 0.098 | 0.144 | 3.13E−13 |
| HCG18 | 2.83E−17 | −0.295 | 0.093 | 0.11 | 3.23E−13 |
| PPME1 | 3.01E−17 | −0.311 | 0.109 | 0.135 | 3.43E−13 |
| S100A4 | 4.81E−17 | −0.577 | 0.086 | 0.121 | 5.48E−13 |
| C2orf49 | 6.87E−17 | −0.344 | 0.107 | 0.126 | 7.83E−13 |
| ASPM | 1.80E−16 | −0.799 | 0.09 | 0.135 | 2.06E−12 |
| RAD51AP1 | 1.88E−16 | −0.254 | 0.111 | 0.094 | 2.15E−12 |
| ADSL | 2.80E−16 | −0.270 | 0.094 | 0.103 | 3.19E−12 |
| ZBTB10 | 2.91E−16 | −0.402 | 0.028 | 0.102 | 3.32E−12 |
| MAFB | 3.03E−16 | −0.311 | 0.113 | 0.12 | 3.46E−12 |
| HPS5 | 5.43E−16 | −0.303 | 0.07 | 0.106 | 6.19E−12 |
| HLA-DRB1 | 5.62E−16 | −0.567 | 0.069 | 0.119 | 6.41E−12 |
| CAMK2G | 7.34E−16 | −0.329 | 0.059 | 0.109 | 8.36E−12 |
| ABCA5 | 1.94E−15 | −0.353 | 0.028 | 0.112 | 2.21E−11 |
| SNHG7 | 3.62E−15 | −0.306 | 0.12 | 0.151 | 4.13E−11 |
| UVRAG | 6.23E−15 | −0.256 | 0.103 | 0.094 | 7.10E−11 |
| EPM2AIP1 | 1.12E−14 | −0.333 | 0.075 | 0.111 | 1.28E−10 |
| RP6-109B7.3 | 2.38E−14 | −0.305 | 0.073 | 0.106 | 2.71E−10 |
| VAV3 | 3.32E−14 | −0.318 | 0.073 | 0.115 | 3.78E−10 |
| CDK1 | 2.30E−13 | −0.293 | 0.185 | 0.161 | 2.62E−09 |
| FAM111B | 1.47E−11 | −0.415 | 0.123 | 0.133 | 1.67E−07 |
| OXSM | 1.81E−11 | −0.257 | 0.058 | 0.106 | 2.06E−07 |
| CCNB1 | 2.75E−07 | −0.329 | 0.135 | 0.128 | 3.13E−03 |
| TOP2A | 9.72E−07 | −0.468 | 0.188 | 0.224 | 1.11E−02 |

CSF.BMvLMD.tumor.bimod.markers.up.1

| | | | | | |
|---|---|---|---|---|---|
| TFF1 | 0.00E+00 | 5.365 | 0.995 | 0.022 | 0.00E+00 |
| TFF3 | 0.00E+00 | 4.231 | 0.978 | 0.072 | 0.00E+00 |
| AGR2 | 0.00E+00 | 4.061 | 0.999 | 0.187 | 0.00E+00 |
| S100P | 0.00E+00 | 3.135 | 0.908 | 0.015 | 0.00E+00 |
| GSTP1 | 0.00E+00 | 2.924 | 0.992 | 0.313 | 0.00E+00 |
| HSPB1 | 0.00E+00 | 2.638 | 0.994 | 0.34 | 0.00E+00 |
| ID3 | 0.00E+00 | 2.267 | 0.856 | 0.054 | 0.00E+00 |
| MUC1 | 0.00E+00 | 2.248 | 0.93 | 0.043 | 0.00E+00 |
| KRT18 | 0.00E+00 | 2.133 | 0.985 | 0.277 | 0.00E+00 |
| ZG16B | 0.00E+00 | 2.128 | 0.928 | 0.056 | 0.00E+00 |
| SMIM22 | 0.00E+00 | 2.092 | 0.931 | 0.084 | 0.00E+00 |
| TSTD1 | 0.00E+00 | 2.067 | 0.989 | 0.329 | 0.00E+00 |
| FXYD3 | 0.00E+00 | 1.973 | 0.911 | 0.116 | 0.00E+00 |
| CDK2AP2 | 0.00E+00 | 1.779 | 0.877 | 0.134 | 0.00E+00 |
| AQP3 | 0.00E+00 | 1.749 | 0.742 | 0.009 | 0.00E+00 |
| HINT1 | 0.00E+00 | 1.522 | 0.99 | 0.535 | 0.00E+00 |
| COX7C | 0.00E+00 | 1.449 | 0.998 | 0.68 | 0.00E+00 |
| IFI27 | 0.00E+00 | 1.410 | 0.991 | 0.242 | 0.00E+00 |
| UQCRQ | 4.24E−322 | 1.405 | 0.991 | 0.541 | 4.84E−318 |
| KRT8 | 3.68E−319 | 1.631 | 0.969 | 0.313 | 4.20E−315 |
| KRT19 | 1.30E−318 | 1.652 | 0.972 | 0.283 | 1.48E−314 |
| ID1 | 3.44E−318 | 1.668 | 0.806 | 0.076 | 3.92E−314 |
| PPP1CA | 6.75E−318 | 1.446 | 0.951 | 0.28 | 7.69E−314 |
| SLC12A2 | 1.37E−317 | 1.828 | 0.911 | 0.178 | 1.56E−313 |
| BST2 | 7.46E−312 | 1.072 | 0.844 | 0.116 | 8.51E−308 |
| TPT1 | 9.55E−310 | 1.077 | 1 | 0.861 | 1.09E−305 |
| SDR16C5 | 0.00E+00 | 1.195 | 0.64 | 0.001 | 3.45E−305 |
| S100A13 | 2.64E−306 | 1.412 | 0.958 | 0.261 | 3.00E−302 |
| TACSTD2 | 5.87E−303 | 1.324 | 0.951 | 0.263 | 6.69E−299 |
| CD63 | 7.90E−301 | 1.246 | 0.975 | 0.437 | 9.00E−297 |
| SAR1B | 2.56E−286 | 1.193 | 0.84 | 0.141 | 2.91E−282 |
| NFKBIA | 3.40E−282 | 1.663 | 0.914 | 0.25 | 3.88E−278 |
| IFITM3 | 1.61E−279 | 0.411 | 0.859 | 0.212 | 1.84E−275 |
| EZR | 1.79E−279 | 1.485 | 0.968 | 0.392 | 2.04E−275 |
| CLDN7 | 6.82E−279 | 1.394 | 0.838 | 0.146 | 7.77E−275 |
| NUDT8 | 6.18E−278 | 1.590 | 0.804 | 0.11 | 7.04E−274 |
| CLDN4 | 6.56E−278 | 1.478 | 0.96 | 0.301 | 7.47E−274 |
| PYCARD | 1.49E−274 | 1.203 | 0.753 | 0.074 | 1.70E−270 |
| MYL6 | 5.09E−269 | 1.119 | 0.995 | 0.683 | 5.81E−265 |
| TSPAN13 | 4.64E−268 | 1.241 | 0.869 | 0.197 | 5.29E−264 |
| C19orf33 | 1.20E−266 | 1.190 | 0.621 | 0.014 | 1.37E−262 |
| GDF15 | 1.62E−266 | 2.202 | 0.619 | 0.014 | 1.85E−262 |
| AGR3 | 1.89E−265 | 1.533 | 0.799 | 0.116 | 2.15E−261 |
| TIMP1 | 4.66E−264 | 1.200 | 0.745 | 0.079 | 5.31E−260 |
| ELF3 | 1.41E−260 | 1.189 | 0.958 | 0.345 | 1.61E−256 |
| TNFRSF12A | 4.76E−257 | 1.218 | 0.865 | 0.183 | 5.42E−253 |
| CTTN | 1.11E−255 | 0.488 | 0.887 | 0.259 | 1.26E−251 |
| TMEM134 | 1.41E−255 | 1.263 | 0.79 | 0.131 | 1.60E−251 |
| CD46 | 1.10E−251 | 1.064 | 0.939 | 0.341 | 1.25E−247 |
| ARHGDIB | 3.21E−250 | 0.943 | 0.88 | 0.224 | 3.66E−246 |
| ALG13 | 5.23E−248 | 0.889 | 0.69 | 0.07 | 5.96E−244 |
| HSP90AA1 | 5.94E−245 | 0.911 | 1 | 0.91 | 6.77E−241 |
| JTB | 1.18E−244 | 1.055 | 0.934 | 0.326 | 1.35E−240 |
| NOSTRIN | 3.76E−241 | 1.200 | 0.583 | 0.014 | 4.29E−237 |
| GPR160 | 4.72E−235 | 1.160 | 0.746 | 0.11 | 5.38E−231 |
| NADSYN1 | 1.08E−233 | 0.999 | 0.692 | 0.077 | 1.23E−229 |
| CAMK2D | 6.60E−231 | 1.138 | 0.704 | 0.084 | 7.52E−227 |
| WASL | 2.54E−230 | 1.157 | 0.809 | 0.17 | 2.90E−226 |
| SYAP1 | 4.41E−230 | 0.838 | 0.849 | 0.221 | 5.03E−226 |
| SYPL1 | 1.04E−229 | 0.999 | 0.837 | 0.209 | 1.19E−225 |
| REEP5 | 1.51E−229 | 0.826 | 0.889 | 0.259 | 1.72E−225 |
| S100A14 | 2.06E−229 | 0.890 | 0.872 | 0.229 | 2.35E−225 |
| SRSF5 | 1.09E−227 | 0.966 | 0.892 | 0.282 | 1.25E−223 |
| TCEB2 | 1.80E−226 | 0.957 | 0.967 | 0.456 | 2.05E−222 |
| IFI27L2 | 6.60E−226 | 0.814 | 0.623 | 0.052 | 7.52E−222 |
| NDUFV1 | 2.23E−224 | 0.913 | 0.781 | 0.159 | 2.54E−220 |
| BMPR1B | 3.72E−224 | 1.314 | 0.64 | 0.053 | 4.23E−220 |
| DUSP4 | 3.77E−224 | 0.940 | 0.893 | 0.275 | 4.30E−220 |
| NDUFS8 | 4.60E−223 | 0.850 | 0.783 | 0.163 | 5.24E−219 |
| ENSA | 4.71E−223 | 1.035 | 0.921 | 0.335 | 5.37E−219 |
| PRSS8 | 2.47E−222 | 0.943 | 0.659 | 0.067 | 2.82E−218 |
| EPCAM | 2.82E−222 | 0.860 | 0.816 | 0.189 | 3.22E−218 |
| MACC1 | 8.53E−221 | 1.038 | 0.755 | 0.131 | 9.73E−217 |
| RPS6KB2 | 1.63E−219 | 0.955 | 0.664 | 0.075 | 1.85E−215 |
| RPS14 | 2.63E−218 | 0.671 | 1 | 0.909 | 3.00E−214 |
| PAM | 8.82E−218 | 1.210 | 0.615 | 0.046 | 1.00E−213 |
| NDUFC2 | 9.85E−217 | 0.987 | 0.969 | 0.429 | 1.12E−212 |
| TMCO1 | 1.23E−213 | 0.600 | 0.922 | 0.348 | 1.40E−209 |
| NDUFC1 | 4.95E−213 | 0.812 | 0.854 | 0.249 | 5.65E−209 |
| PFDN5 | 3.03E−212 | 1.043 | 0.977 | 0.519 | 3.45E−208 |
| RAB25 | 4.63E−212 | 0.921 | 0.61 | 0.049 | 5.28E−208 |
| NDUFB2 | 1.75E−210 | 0.809 | 0.965 | 0.437 | 1.99E−206 |
| ADIPOR1 | 2.57E−210 | 0.768 | 0.779 | 0.183 | 2.93E−206 |
| POLR2L | 3.31E−210 | 0.835 | 0.927 | 0.344 | 3.77E−206 |
| GLUL | 2.33E−209 | 0.820 | 0.817 | 0.207 | 2.65E−205 |
| ATP5G2 | 2.98E−209 | 0.947 | 0.94 | 0.386 | 3.40E−205 |
| FBP1 | 6.43E−208 | 0.681 | 0.564 | 0.047 | 7.33E−204 |
| PHLDA2 | 1.37E−207 | 1.050 | 0.673 | 0.084 | 1.57E−203 |
| NDUFA4 | 2.03E−207 | 1.065 | 0.99 | 0.694 | 2.31E−203 |
| DAZAP2 | 9.90E−207 | 0.635 | 0.748 | 0.17 | 1.13E−202 |
| NDUFB10 | 3.25E−206 | 0.787 | 0.874 | 0.278 | 3.70E−202 |
| GSTK1 | 4.86E−206 | 0.753 | 0.666 | 0.1 | 5.54E−202 |
| ATOX1 | 1.16E−205 | 0.731 | 0.736 | 0.15 | 1.32E−201 |
| PLK2 | 6.46E−205 | 1.023 | 0.794 | 0.183 | 7.37E−201 |
| ATPIF1 | 7.74E−204 | 0.744 | 0.874 | 0.281 | 8.82E−200 |
| NTN4 | 2.70E−201 | 0.930 | 0.554 | 0.032 | 3.07E−197 |
| CD151 | 4.25E−201 | 0.848 | 0.709 | 0.129 | 4.85E−197 |
| IRF1 | 2.54E−200 | 0.939 | 0.602 | 0.057 | 2.90E−196 |
| CTSD | 3.33E−200 | 0.950 | 0.843 | 0.246 | 3.80E−196 |
| PSME1 | 6.73E−200 | 0.739 | 0.858 | 0.278 | 7.67E−196 |
| BTF3 | 1.71E−198 | 0.878 | 0.977 | 0.532 | 1.95E−194 |
| ATP5J | 1.83E−198 | 0.701 | 0.921 | 0.365 | 2.08E−194 |
| CCND1 | 2.42E−198 | 1.111 | 0.9 | 0.313 | 2.75E−194 |
| ZNHIT1 | 8.27E−198 | 0.579 | 0.804 | 0.232 | 9.42E−194 |
| KRT7 | 2.80E−197 | 1.286 | 0.963 | 0.471 | 3.19E−193 |
| MIA3 | 4.15E−197 | 0.896 | 0.84 | 0.246 | 4.74E−193 |
| TMEM50B | 2.44E−196 | 0.857 | 0.644 | 0.086 | 2.78E−192 |
| PERP | 2.52E−196 | 0.854 | 0.908 | 0.338 | 2.87E−192 |
| UBC | 2.76E−196 | 0.970 | 0.985 | 0.576 | 3.15E−192 |
| CD164 | 3.09E−196 | 0.571 | 0.875 | 0.311 | 3.52E−192 |
| ATP6V1G1 | 4.03E−196 | 0.813 | 0.954 | 0.415 | 4.59E−192 |
| RPS27L | 7.87E−196 | 1.052 | 0.895 | 0.316 | 8.97E−192 |
| SSFA2 | 7.74E−194 | 1.149 | 0.683 | 0.107 | 8.82E−190 |
| TXNIP | 7.88E−194 | 1.161 | 0.829 | 0.229 | 8.99E−190 |
| SLC44A4 | 8.06E−194 | 0.834 | 0.512 | 0.019 | 9.19E−190 |
| AAMDC | 8.26E−194 | 0.941 | 0.725 | 0.138 | 9.41E−190 |
| RAB9A | 8.66E−192 | 0.802 | 0.609 | 0.075 | 9.88E−188 |
| SBDS | 2.18E−190 | 0.563 | 0.787 | 0.226 | 2.48E−186 |
| NDUFA2 | 1.15E−188 | 0.723 | 0.851 | 0.28 | 1.31E−184 |
| ERBB3 | 3.87E−188 | 0.728 | 0.682 | 0.132 | 4.41E−184 |
| GUK1 | 1.46E−187 | 0.395 | 0.912 | 0.39 | 1.67E−183 |
| SMIM14 | 3.68E−187 | 0.677 | 0.835 | 0.256 | 4.20E−183 |
| BLVRB | 9.47E−187 | 0.810 | 0.71 | 0.143 | 1.08E−182 |
| BUD31 | 2.95E−185 | 0.779 | 0.749 | 0.181 | 3.36E−181 |
| AKAP9 | 5.04E−184 | 0.854 | 0.944 | 0.421 | 5.74E−180 |
| TMBIM6 | 2.52E−183 | 0.760 | 0.973 | 0.551 | 2.88E−179 |
| SRP14 | 9.83E−183 | 0.637 | 0.933 | 0.425 | 1.12E−178 |
| NDUFA1 | 1.59E−182 | 0.790 | 0.954 | 0.468 | 1.81E−178 |
| CAPN2 | 1.85E−182 | 0.385 | 0.821 | 0.285 | 2.11E−178 |
| C1orf43 | 8.14E−182 | 0.646 | 0.836 | 0.271 | 9.28E−178 |
| ATP5O | 1.08E−181 | 0.785 | 0.854 | 0.291 | 1.23E−177 |
| SDC4 | 1.25E−181 | 1.013 | 0.698 | 0.13 | 1.42E−177 |
| ATP1B1 | 4.25E−181 | 1.045 | 0.855 | 0.279 | 4.84E−177 |
| CLU | 6.76E−181 | 0.989 | 0.84 | 0.261 | 7.70E−177 |
| C15orf48 | 1.00E−180 | 0.875 | 0.515 | 0.037 | 1.14E−176 |
| ARPC1B | 1.89E−180 | 0.302 | 0.725 | 0.221 | 2.16E−176 |
| MPC2 | 2.74E−180 | 0.543 | 0.747 | 0.206 | 3.13E−176 |
| RER1 | 1.38E−179 | 0.463 | 0.675 | 0.161 | 1.57E−175 |
| ADAM15 | 4.32E−179 | 0.693 | 0.679 | 0.141 | 4.92E−175 |
| IFNAR1 | 2.46E−178 | 0.531 | 0.691 | 0.164 | 2.80E−174 |
| DNAJC1 | 2.63E−178 | 0.804 | 0.781 | 0.211 | 3.00E−174 |
| LGALS3 | 2.95E−178 | 1.068 | 0.935 | 0.428 | 3.36E−174 |
| NME3 | 6.22E−178 | 0.747 | 0.656 | 0.118 | 7.09E−174 |
| TPM3 | 1.29E−177 | 0.546 | 0.963 | 0.492 | 1.47E−173 |
| FAM134B | 2.31E−177 | 0.707 | 0.541 | 0.054 | 2.64E−173 |
| RSF1 | 7.22E−177 | 0.718 | 0.926 | 0.389 | 8.23E−173 |
| RHOC | 2.05E−176 | 0.750 | 0.755 | 0.201 | 2.34E−172 |
| COPZ1 | 2.11E−176 | 0.628 | 0.772 | 0.225 | 2.41E−172 |
| SAT1 | 4.89E−176 | 1.023 | 0.938 | 0.427 | 5.57E−172 |
| LASP1 | 6.40E−176 | 0.621 | 0.557 | 0.072 | 7.29E−172 |
| PDIA4 | 6.61E−176 | 0.816 | 0.834 | 0.274 | 7.53E−172 |
| ATP5B | 8.42E−176 | 0.511 | 0.861 | 0.329 | 9.60E−172 |
| CBR3 | 1.75E−175 | 0.917 | 0.526 | 0.038 | 2.00E−171 |
| BTG2 | 2.16E−175 | 0.887 | 0.701 | 0.147 | 2.46E−171 |
| DHCR7 | 2.79E−175 | 0.968 | 0.733 | 0.169 | 3.18E−171 |
| S100A16 | 6.94E−175 | 0.708 | 0.776 | 0.214 | 7.90E−171 |
| KDELR2 | 1.82E−174 | 0.631 | 0.837 | 0.29 | 2.07E−170 |
| TCEA3 | 4.45E−174 | 0.719 | 0.626 | 0.106 | 5.07E−170 |
| KRAS | 5.85E−174 | 0.666 | 0.706 | 0.166 | 6.66E−170 |
| SAP18 | 6.68E−174 | 0.355 | 0.779 | 0.266 | 7.61E−170 |
| LMAN2 | 6.35E−173 | 0.509 | 0.788 | 0.257 | 7.23E−169 |
| RGS10 | 8.35E−173 | 0.371 | 0.756 | 0.24 | 9.52E−169 |
| CLINT1 | 8.23E−172 | 0.782 | 0.768 | 0.215 | 9.38E−168 |
| PPP1R15A | 1.17E−171 | 0.873 | 0.8 | 0.236 | 1.33E−167 |
| KLF4 | 1.98E−170 | 0.608 | 0.584 | 0.093 | 2.26E−166 |
| ZCRB1 | 2.24E−170 | 0.354 | 0.746 | 0.242 | 2.55E−166 |
| CREG1 | 2.40E−170 | 0.519 | 0.69 | 0.175 | 2.73E−166 |
| SHFM1 | 2.48E−170 | 0.669 | 0.847 | 0.303 | 2.83E−166 |
| CA12 | 6.60E−170 | 0.934 | 0.682 | 0.135 | 7.52E−166 |
| RALGPS2 | 8.09E−170 | 0.730 | 0.621 | 0.103 | 9.22E−166 |
| LYPLA1 | 2.17E−169 | 0.488 | 0.768 | 0.243 | 2.47E−165 |
| SKP1 | 2.56E−169 | 0.669 | 0.953 | 0.489 | 2.92E−165 |
| MYO1B | 4.16E−169 | 0.897 | 0.52 | 0.04 | 4.75E−165 |
| GNG5 | 8.10E−169 | 0.384 | 0.745 | 0.237 | 9.24E−165 |
| NELL2 | 9.86E−168 | 0.960 | 0.461 | 0.018 | 1.12E−163 |
| NUCB2 | 5.07E−167 | 0.675 | 0.765 | 0.222 | 5.77E−163 |
| TSTA3 | 5.52E−167 | 0.665 | 0.659 | 0.143 | 6.29E−163 |
| SQSTM1 | 5.98E−167 | 1.118 | 0.915 | 0.39 | 6.82E−163 |
| ARL1 | 6.83E−167 | 0.608 | 0.707 | 0.179 | 7.79E−163 |
| SEMA3C | 7.78E−167 | 0.681 | 0.643 | 0.126 | 8.87E−163 |
| CNPY2 | 1.50E−166 | 0.574 | 0.709 | 0.191 | 1.71E−162 |
| ELL2 | 3.13E−166 | 0.569 | 0.558 | 0.085 | 3.57E−162 |
| CREB3L4 | 4.09E−166 | 0.749 | 0.646 | 0.124 | 4.66E−162 |
| IL6ST | 2.08E−165 | 0.556 | 0.698 | 0.179 | 2.37E−161 |
| BLOC1S1 | 3.40E−165 | 0.556 | 0.621 | 0.127 | 3.88E−161 |
| TUFM | 3.51E−165 | 0.524 | 0.72 | 0.206 | 4.00E−161 |
| PLCB1 | 3.75E−165 | 0.806 | 0.489 | 0.031 | 4.27E−161 |
| CLDN3 | 4.93E−165 | 1.001 | 0.779 | 0.222 | 5.62E−161 |
| ID2 | 5.80E−165 | 0.864 | 0.881 | 0.343 | 6.61E−161 |
| PPP2CA | 8.58E−165 | 0.556 | 0.712 | 0.198 | 9.77E−161 |
| CXCL16 | 1.04E−164 | 0.662 | 0.568 | 0.082 | 1.18E−160 |
| TUFT1 | 1.22E−164 | 0.935 | 0.479 | 0.028 | 1.38E−160 |
| CDKN1A | 2.52E−164 | 1.250 | 0.6 | 0.086 | 2.87E−160 |
| SUCO | 3.38E−164 | 0.676 | 0.771 | 0.233 | 3.85E−160 |
| RP11-532F12.5 | 4.95E−164 | 0.674 | 0.479 | 0.033 | 5.64E−160 |
| CHCHD2 | 8.89E−164 | 0.575 | 0.918 | 0.424 | 1.01E−159 |
| C11orf24 | 1.32E−163 | 0.573 | 0.458 | 0.034 | 1.50E−159 |
| DENND1B | 6.34E−163 | 0.904 | 0.666 | 0.135 | 7.23E−159 |
| NDUFAB1 | 6.78E−163 | 0.601 | 0.813 | 0.284 | 7.73E−159 |
| CSRP1 | 8.12E−163 | 0.682 | 0.693 | 0.167 | 9.25E−159 |
| RPS15A | 8.65E−163 | 0.616 | 0.998 | 0.826 | 9.86E−159 |
| WDR1 | 1.19E−162 | 0.545 | 0.645 | 0.143 | 1.36E−158 |
| TBC1D9 | 1.77E−162 | 0.416 | 0.543 | 0.097 | 2.02E−158 |
| RPL27 | 1.92E−162 | 0.479 | 1 | 0.82 | 2.19E−158 |
| NDUFV3 | 2.88E−162 | 0.356 | 0.618 | 0.157 | 3.28E−158 |
| RHOB | 3.56E−162 | 0.770 | 0.826 | 0.279 | 4.06E−158 |
| SOD1 | 6.14E−162 | 0.568 | 0.875 | 0.357 | 6.99E−158 |
| GNB2 | 4.05E−161 | 0.648 | 0.653 | 0.144 | 4.62E−157 |
| PSME2 | 6.00E−161 | 0.268 | 0.658 | 0.19 | 6.83E−157 |
| KIAA1244 | 1.32E−160 | 0.618 | 0.668 | 0.155 | 1.50E−156 |
| RPS4X | 1.77E−160 | 0.823 | 0.992 | 0.752 | 2.02E−156 |
| COX5A | 3.13E−160 | 0.540 | 0.842 | 0.317 | 3.57E−156 |
| TMEM219 | 4.77E−160 | 0.496 | 0.559 | 0.101 | 5.44E−156 |
| PDLIM5 | 8.92E−160 | 0.345 | 0.645 | 0.169 | 1.02E−155 |
| FUS | 1.58E−159 | 0.522 | 0.839 | 0.315 | 1.80E−155 |
| SLC25A3 | 1.83E−159 | 0.367 | 0.804 | 0.305 | 2.09E−155 |
| CDC42 | 2.08E−159 | 0.495 | 0.869 | 0.361 | 2.37E−155 |
| ATP2A3 | 5.81E−159 | 0.678 | 0.421 | 0.012 | 6.62E−155 |
| CIB1 | 4.08E−158 | 0.335 | 0.618 | 0.163 | 4.65E−154 |
| ITM2B | 6.84E−158 | 0.285 | 0.803 | 0.317 | 7.80E−154 |
| EIF5 | 1.80E−157 | 0.617 | 0.93 | 0.446 | 2.05E−153 |
| BLVRA | 1.90E−157 | 0.604 | 0.582 | 0.101 | 2.16E−153 |
| PSMB1 | 7.48E−157 | 0.283 | 0.796 | 0.313 | 8.53E−153 |
| NDUFA5 | 9.11E−157 | 0.388 | 0.727 | 0.232 | 1.04E−152 |
| UAP1 | 3.57E−156 | 0.384 | 0.673 | 0.191 | 4.07E−152 |
| S100A11 | 8.36E−156 | 0.729 | 0.987 | 0.647 | 9.53E−152 |
| ARF4 | 8.42E−156 | 0.481 | 0.669 | 0.179 | 9.60E−152 |
| GADD45B | 8.44E−156 | 0.606 | 0.795 | 0.265 | 9.63E−152 |
| SEC61G | 8.64E−156 | 0.445 | 0.782 | 0.275 | 9.85E−152 |
| ANXA6 | 2.00E−155 | 0.597 | 0.585 | 0.108 | 2.28E−151 |
| UQCRC2 | 5.42E−155 | 0.526 | 0.609 | 0.135 | 6.18E−151 |
| FAM107B | 9.01E−155 | 0.724 | 0.56 | 0.084 | 1.03E−150 |
| MRPS34 | 1.04E−154 | 0.300 | 0.739 | 0.263 | 1.19E−150 |
| DDIT3 | 1.39E−154 | 0.791 | 0.625 | 0.12 | 1.59E−150 |
| IFITM2 | 2.50E−154 | 0.328 | 0.531 | 0.11 | 2.85E−150 |
| MCL1 | 2.71E−154 | 0.526 | 0.787 | 0.271 | 3.09E−150 |
| DPM3 | 3.10E−154 | 0.395 | 0.722 | 0.235 | 3.53E−150 |
| ADAM8 | 3.62E−154 | 0.577 | 0.392 | 0.012 | 4.12E−150 |
| GPRC5A | 5.33E−154 | 1.007 | 0.573 | 0.081 | 6.07E−150 |
| XIST | 1.79E−153 | 0.299 | 0.577 | 0.137 | 2.04E−149 |
| ELF1 | 2.06E−153 | 0.376 | 0.54 | 0.11 | 2.35E−149 |
| PBX1 | 3.35E−153 | 0.653 | 0.81 | 0.284 | 3.82E−149 |
| ATP6V0E1 | 4.30E−153 | 0.453 | 0.689 | 0.199 | 4.90E−149 |
| GALNT3 | 4.68E−153 | 0.798 | 0.638 | 0.129 | 5.34E−149 |
| CAST | 5.71E−153 | 0.635 | 0.872 | 0.356 | 6.51E−149 |
| LITAF | 6.04E−153 | 0.498 | 0.74 | 0.234 | 6.88E−149 |
| CST3 | 7.78E−153 | 0.439 | 0.732 | 0.233 | 8.87E−149 |
| MGMT | 1.09E−152 | 0.508 | 0.44 | 0.038 | 1.24E−148 |
| KIAA1522 | 2.02E−152 | 0.412 | 0.513 | 0.089 | 2.30E−148 |
| FAM114A1 | 2.25E−152 | 0.515 | 0.482 | 0.06 | 2.57E−148 |
| GALNT7 | 2.48E−152 | 0.453 | 0.666 | 0.183 | 2.82E−148 |
| ATP5G3 | 6.17E−152 | 0.560 | 0.886 | 0.389 | 7.03E−148 |
| ATP5J2 | 1.45E−151 | 0.405 | 0.876 | 0.389 | 1.66E−147 |
| TMEM159 | 2.65E−151 | 0.760 | 0.642 | 0.135 | 3.02E−147 |
| VDAC1 | 2.85E−151 | 0.252 | 0.756 | 0.287 | 3.25E−147 |
| MARVELD2 | 4.31E−151 | 0.634 | 0.476 | 0.047 | 4.91E−147 |
| GALNT6 | 5.92E−151 | 0.689 | 0.557 | 0.086 | 6.75E−147 |
| PTTG1IP | 8.84E−151 | 0.276 | 0.918 | 0.471 | 1.01E−146 |
| JUP | 9.46E−151 | 0.473 | 0.686 | 0.195 | 1.08E−146 |
| TMEM9B | 1.05E−150 | 0.378 | 0.516 | 0.096 | 1.20E−146 |
| UHMK1 | 1.19E−150 | 0.293 | 0.629 | 0.184 | 1.35E−146 |
| EFCAB4A | 2.05E−150 | 0.675 | 0.486 | 0.049 | 2.34E−146 |
| OCLN | 2.19E−150 | 0.480 | 0.412 | 0.03 | 2.50E−146 |
| ATP5G1 | 6.64E−150 | 0.307 | 0.646 | 0.195 | 7.57E−146 |
| CNN3 | 9.94E−150 | 0.605 | 0.729 | 0.214 | 1.13E−145 |
| TUBA1C | 5.87E−149 | 0.434 | 0.779 | 0.276 | 6.69E−145 |
| BCAP29 | 9.15E−149 | 0.327 | 0.559 | 0.129 | 1.04E−144 |
| TIPARP | 1.05E−148 | 0.593 | 0.559 | 0.097 | 1.19E−144 |
| PTP4A2 | 1.23E−148 | 0.443 | 0.806 | 0.308 | 1.40E−144 |
| RPL36AL | 3.44E−148 | 0.316 | 0.855 | 0.38 | 3.92E−144 |
| CSNK1A1 | 6.57E−148 | 0.393 | 0.839 | 0.346 | 7.48E−144 |
| KLF5 | 7.96E−148 | 0.396 | 0.408 | 0.04 | 9.07E−144 |
| ALDH9A1 | 8.91E−148 | 0.402 | 0.567 | 0.129 | 1.02E−143 |
| EDF1 | 1.41E−147 | 0.375 | 0.859 | 0.37 | 1.61E−143 |
| RASSF7 | 6.02E−147 | 0.593 | 0.456 | 0.041 | 6.86E−143 |
| TMED4 | 1.23E−146 | 0.446 | 0.679 | 0.201 | 1.40E−142 |
| CPEB2 | 6.33E−146 | 0.665 | 0.428 | 0.028 | 7.21E−142 |
| COX7A2 | 7.04E−146 | 0.565 | 0.979 | 0.62 | 8.02E−142 |
| COX14 | 9.98E−146 | 0.549 | 0.674 | 0.186 | 1.14E−141 |
| PYCR2 | 1.65E−145 | 0.349 | 0.491 | 0.09 | 1.88E−141 |
| MAPKAPK2 | 2.32E−145 | 0.508 | 0.627 | 0.156 | 2.64E−141 |
| STARD10 | 2.38E−145 | 0.714 | 0.651 | 0.149 | 2.72E−141 |
| COX7B | 2.59E−145 | 0.397 | 0.902 | 0.431 | 2.95E−141 |
| PTPLAD1 | 5.69E−145 | 0.424 | 0.797 | 0.299 | 6.49E−141 |
| FOXP1 | 5.76E−145 | 0.685 | 0.634 | 0.141 | 6.57E−141 |
| PPA1 | 7.38E−145 | 0.445 | 0.906 | 0.426 | 8.41E−141 |
| RAB2A | 1.04E−144 | 0.393 | 0.754 | 0.275 | 1.19E−140 |
| CKAP4 | 1.70E−144 | 0.492 | 0.568 | 0.119 | 1.94E−140 |
| TAF7 | 4.49E−144 | 0.408 | 0.722 | 0.242 | 5.12E−140 |
| LRP10 | 9.37E−144 | 0.305 | 0.501 | 0.105 | 1.07E−139 |
| CMTM6 | 1.12E−143 | 0.379 | 0.644 | 0.183 | 1.28E−139 |
| ARPC1A | 1.35E−143 | 0.344 | 0.514 | 0.109 | 1.54E−139 |
| LMNA | 1.62E−143 | 0.448 | 0.859 | 0.374 | 1.84E−139 |
| CORO2A | 4.72E−143 | 0.629 | 0.422 | 0.03 | 5.38E−139 |
| DYNLT1 | 6.98E−143 | 0.300 | 0.687 | 0.232 | 7.96E−139 |
| MRPL21 | 1.13E−142 | 0.538 | 0.67 | 0.186 | 1.29E−138 |
| PVRL4 | 1.14E−142 | 0.616 | 0.411 | 0.022 | 1.30E−138 |
| CAPZA2 | 4.00E−142 | 0.253 | 0.644 | 0.211 | 4.56E−138 |
| MRPS21 | 4.96E−142 | 0.387 | 0.729 | 0.253 | 5.65E−138 |
| SPCS3 | 5.78E−142 | 0.463 | 0.684 | 0.209 | 6.59E−138 |
| EMP2 | 7.32E−142 | 0.714 | 0.717 | 0.21 | 8.35E−138 |
| CDS1 | 7.78E−142 | 0.366 | 0.455 | 0.073 | 8.87E−138 |
| RPL37A | 1.40E−141 | 0.406 | 1 | 0.897 | 1.60E−137 |
| NDUFS7 | 1.67E−141 | 0.345 | 0.592 | 0.158 | 1.91E−137 |
| RND1 | 2.88E−141 | 0.748 | 0.46 | 0.041 | 3.28E−137 |
| COX6A1 | 5.92E−141 | 0.428 | 0.974 | 0.567 | 6.75E−137 |
| UBE2B | 8.90E−141 | 0.539 | 0.636 | 0.163 | 1.01E−136 |
| RAB11A | 1.41E−140 | 0.426 | 0.663 | 0.198 | 1.61E−136 |
| CERS2 | 1.51E−140 | 0.435 | 0.604 | 0.154 | 1.72E−136 |
| HIGD2A | 2.08E−140 | 0.414 | 0.625 | 0.174 | 2.37E−136 |
| TMEM50A | 2.88E−140 | 0.313 | 0.638 | 0.2 | 3.28E−136 |
| C14orf2 | 3.17E−140 | 0.593 | 0.965 | 0.56 | 3.62E−136 |
| PJA2 | 3.48E−140 | 0.363 | 0.584 | 0.152 | 3.97E−136 |
| ARSD | 4.15E−140 | 0.476 | 0.442 | 0.054 | 4.73E−136 |
| PON2 | 5.17E−140 | 0.543 | 0.496 | 0.075 | 5.89E−136 |
| MAGED2 | 5.92E−140 | 0.590 | 0.713 | 0.22 | 6.75E−136 |
| RPL41 | 7.38E−140 | 0.523 | 0.997 | 0.843 | 8.41E−136 |
| MX1 | 7.67E−140 | 0.252 | 0.588 | 0.162 | 8.75E−136 |
| NR2F2 | 8.31E−140 | 0.518 | 0.511 | 0.085 | 9.47E−136 |
| PDXDC1 | 1.06E−139 | 0.419 | 0.732 | 0.254 | 1.20E−135 |
| EVA1C | 1.16E−139 | 0.666 | 0.39 | 0.013 | 1.33E−135 |
| COMT | 4.96E−139 | 0.610 | 0.719 | 0.221 | 5.66E−135 |
| TOMM7 | 5.09E−139 | 0.462 | 0.951 | 0.518 | 5.80E−135 |
| DUSP1 | 5.15E−139 | 0.531 | 0.743 | 0.245 | 5.87E−135 |
| PMM2 | 1.20E−138 | 0.448 | 0.525 | 0.106 | 1.37E−134 |
| CD2AP | 1.36E−138 | 0.351 | 0.612 | 0.171 | 1.56E−134 |
| POC1B-GALNT4 | 1.53E−138 | 0.675 | 0.365 | 0.006 | 1.74E−134 |
| MT-TF | 1.57E−138 | 0.830 | 0.567 | 0.097 | 1.79E−134 |
| MT1E | 1.71E−138 | 0.681 | 0.395 | 0.021 | 1.95E−134 |
| KRT23 | 1.84E−138 | 0.680 | 0.394 | 0.017 | 2.10E−134 |
| ALDOA | 1.95E−138 | 0.566 | 0.967 | 0.566 | 2.22E−134 |
| MDK | 2.06E−138 | 0.569 | 0.642 | 0.167 | 2.35E−134 |
| LSMD1 | 2.38E−138 | 0.417 | 0.76 | 0.282 | 2.71E−134 |
| CDC42SE1 | 2.39E−138 | 0.359 | 0.623 | 0.186 | 2.72E−134 |
| TBCA | 2.86E−138 | 0.411 | 0.88 | 0.412 | 3.26E−134 |
| ADAM9 | 3.37E−138 | 0.339 | 0.55 | 0.136 | 3.84E−134 |
| SRA1 | 3.81E−138 | 0.541 | 0.543 | 0.105 | 4.34E−134 |
| KIF5B | 9.80E−138 | 0.403 | 0.819 | 0.341 | 1.12E−133 |
| PPP2R2A | 1.61E−137 | 0.349 | 0.594 | 0.16 | 1.84E−133 |
| TMED9 | 1.76E−137 | 0.438 | 0.778 | 0.302 | 2.01E−133 |
| TMEM87A | 1.98E−137 | 0.313 | 0.696 | 0.247 | 2.26E−133 |
| C12orf23 | 6.28E−137 | 0.644 | 0.603 | 0.131 | 7.16E−133 |
| PPP6R3 | 6.31E−137 | 0.497 | 0.571 | 0.127 | 7.20E−133 |
| SSH3 | 8.15E−137 | 0.604 | 0.436 | 0.037 | 9.28E−133 |
| ADIRF | 8.41E−137 | 0.558 | 0.667 | 0.189 | 9.59E−133 |
| CYB5A | 1.59E−136 | 0.303 | 0.678 | 0.228 | 1.81E−132 |
| LIMA1 | 2.95E−136 | 0.764 | 0.581 | 0.112 | 3.36E−132 |
| SSBP1 | 3.01E−136 | 0.285 | 0.824 | 0.361 | 3.43E−132 |
| SON | 3.20E−136 | 0.356 | 0.877 | 0.422 | 3.65E−132 |
| NPDC1 | 1.31E−135 | 0.525 | 0.668 | 0.191 | 1.49E−131 |
| MPC1 | 1.44E−135 | 0.348 | 0.617 | 0.177 | 1.64E−131 |
| LAMTOR5 | 2.03E−135 | 0.292 | 0.693 | 0.247 | 2.32E−131 |
| CIRBP | 2.27E−135 | 0.482 | 0.735 | 0.254 | 2.58E−131 |
| LINC00467 | 3.60E−135 | 0.484 | 0.401 | 0.037 | 4.11E−131 |
| COPA | 4.15E−135 | 0.256 | 0.644 | 0.218 | 4.73E−131 |
| NDUFB1 | 9.35E−135 | 0.396 | 0.911 | 0.454 | 1.07E−130 |
| CALR | 1.35E−134 | 0.763 | 0.972 | 0.644 | 1.54E−130 |
| BCAS1 | 1.59E−134 | 0.792 | 0.418 | 0.027 | 1.82E−130 |
| FCHO2 | 1.91E−134 | 0.535 | 0.422 | 0.04 | 2.17E−130 |
| EPS8 | 2.20E−134 | 0.656 | 0.638 | 0.16 | 2.50E−130 |
| PALLD | 2.21E−134 | 1.544 | 0.612 | 0.135 | 2.52E−130 |
| TMED3 | 4.97E−134 | 0.551 | 0.674 | 0.198 | 5.67E−130 |
| MRPL24 | 5.60E−134 | 0.288 | 0.554 | 0.153 | 6.38E−130 |
| PCBD1 | 5.81E−134 | 0.587 | 0.669 | 0.191 | 6.63E−130 |
| MYO1C | 9.23E−134 | 0.614 | 0.554 | 0.105 | 1.05E−129 |
| GOLM1 | 1.39E−133 | 0.524 | 0.59 | 0.139 | 1.58E−129 |
| C1orf21 | 2.32E−133 | 0.257 | 0.696 | 0.254 | 2.64E−129 |
| MGST2 | 2.81E−133 | 0.575 | 0.491 | 0.074 | 3.21E−129 |
| TMED7 | 3.66E−133 | 0.331 | 0.484 | 0.103 | 4.17E−129 |
| GSPT1 | 3.97E−133 | 0.413 | 0.765 | 0.294 | 4.53E−129 |
| MT-ND3 | 1.42E−132 | 0.541 | 0.988 | 0.697 | 1.62E−128 |
| RAMP1 | 1.50E−132 | 0.603 | 0.345 | 0.005 | 1.71E−128 |
| JUN | 2.21E−132 | 0.520 | 0.929 | 0.478 | 2.52E−128 |
| LAMTOR4 | 3.82E−132 | 0.423 | 0.609 | 0.168 | 4.36E−128 |
| KDM2A | 7.72E−132 | 0.445 | 0.66 | 0.2 | 8.80E−128 |
| MRPS36 | 8.21E−132 | 0.435 | 0.575 | 0.144 | 9.36E−128 |
| CHP1 | 1.21E−131 | 0.254 | 0.497 | 0.124 | 1.38E−127 |
| SIVA1 | 1.26E−131 | 0.356 | 0.77 | 0.306 | 1.43E−127 |
| PHAX | 1.29E−131 | 0.383 | 0.515 | 0.111 | 1.47E−127 |
| SDHB | 2.10E−131 | 0.285 | 0.518 | 0.126 | 2.39E−127 |
| NDUFB3 | 3.21E−131 | 0.311 | 0.66 | 0.221 | 3.66E−127 |
| EIF1AX | 4.81E−131 | 0.262 | 0.697 | 0.262 | 5.49E−127 |
| WDR24 | 9.43E−131 | 0.673 | 0.416 | 0.029 | 1.07E−126 |
| ACSL3 | 1.80E−130 | 0.415 | 0.637 | 0.192 | 2.05E−126 |
| HSPA4 | 4.05E−130 | 0.568 | 0.666 | 0.201 | 4.61E−126 |
| H2AFY | 6.71E−130 | 0.431 | 0.774 | 0.304 | 7.65E−126 |
| HSPA9 | 8.23E−130 | 0.376 | 0.805 | 0.345 | 9.38E−126 |
| SCCPDH | 8.72E−130 | 0.361 | 0.473 | 0.092 | 9.93E−126 |
| PSMA5 | 1.22E−129 | 0.464 | 0.598 | 0.158 | 1.39E−125 |
| MBOAT2 | 2.11E−129 | 0.773 | 0.592 | 0.129 | 2.40E−125 |
| RAP1B | 2.38E−129 | 0.358 | 0.535 | 0.132 | 2.71E−125 |
| OST4 | 2.43E−129 | 0.526 | 0.937 | 0.506 | 2.77E−125 |
| RPLP1 | 2.54E−129 | 0.295 | 1 | 0.959 | 2.90E−125 |
| APOA1BP | 4.88E−129 | 0.298 | 0.584 | 0.175 | 5.56E−125 |
| NQO1 | 1.00E−128 | 0.421 | 0.733 | 0.263 | 1.14E−124 |
| H3F3B | 1.30E−128 | 0.639 | 0.996 | 0.725 | 1.48E−124 |
| TXNDC17 | 2.00E−128 | 0.420 | 0.798 | 0.329 | 2.29E−124 |
| TMEM8B | 3.26E−128 | 0.487 | 0.342 | 0.011 | 3.72E−124 |
| MLEC | 3.99E−128 | 0.285 | 0.726 | 0.283 | 4.55E−124 |
| SPDEF | 5.09E−128 | 0.467 | 0.623 | 0.173 | 5.80E−124 |
| KLF3 | 6.13E−128 | 0.637 | 0.542 | 0.106 | 6.99E−124 |
| CAMLG | 9.01E−128 | 0.410 | 0.62 | 0.182 | 1.03E−123 |
| ACTB | 1.50E−127 | 0.938 | 0.977 | 0.682 | 1.71E−123 |
| EAPP | 1.52E−127 | 0.336 | 0.5 | 0.114 | 1.73E−123 |
| ZCCHC10 | 2.13E−127 | 0.439 | 0.492 | 0.094 | 2.43E−123 |
| EFNA1 | 2.59E−127 | 0.613 | 0.494 | 0.08 | 2.95E−123 |
| SQRDL | 2.78E−127 | 0.271 | 0.385 | 0.062 | 3.17E−123 |
| EPS8L2 | 2.95E−127 | 0.405 | 0.458 | 0.079 | 3.36E−123 |
| SERTAD1 | 4.73E−127 | 0.675 | 0.647 | 0.173 | 5.39E−123 |
| NVL | 4.97E−127 | 0.273 | 0.5 | 0.121 | 5.67E−123 |
| SYTL2 | 5.92E−127 | 0.740 | 0.478 | 0.065 | 6.75E−123 |
| TMEM66 | 7.17E−127 | 0.351 | 0.791 | 0.337 | 8.17E−123 |
| ARF3 | 1.10E−126 | 0.365 | 0.514 | 0.121 | 1.25E−122 |
| RASSF6 | 1.28E−126 | 0.678 | 0.365 | 0.018 | 1.46E−122 |
| SLC39A6 | 2.29E−126 | 0.555 | 0.589 | 0.144 | 2.60E−122 |
| HAX1 | 2.70E−126 | 0.311 | 0.576 | 0.17 | 3.07E−122 |
| CCT2 | 3.91E−126 | 0.308 | 0.703 | 0.266 | 4.46E−122 |
| SEC13 | 4.38E−126 | 0.336 | 0.64 | 0.212 | 5.00E−122 |
| SLU7 | 6.12E−126 | 0.449 | 0.591 | 0.159 | 6.97E−122 |
| PNKD | 6.40E−126 | 0.378 | 0.583 | 0.166 | 7.29E−122 |
| HMG20B | 1.31E−125 | 0.489 | 0.614 | 0.17 | 1.50E−121 |
| EIF2S3 | 1.39E−125 | 0.392 | 0.716 | 0.261 | 1.58E−121 |
| TBK1 | 1.46E−125 | 0.354 | 0.417 | 0.065 | 1.66E−121 |
| RAB13 | 4.01E−125 | 0.479 | 0.652 | 0.199 | 4.57E−121 |
| ICA1 | 4.98E−125 | 0.271 | 0.499 | 0.13 | 5.67E−121 |
| HOXB2 | 6.46E−125 | 0.566 | 0.355 | 0.012 | 7.36E−121 |
| AKAP5 | 9.22E−125 | 0.507 | 0.363 | 0.023 | 1.05E−120 |
| TSPAN3 | 1.05E−124 | 0.647 | 0.626 | 0.167 | 1.20E−120 |
| TAX1BP1 | 1.18E−124 | 0.308 | 0.87 | 0.428 | 1.35E−120 |
| CERS6 | 1.31E−124 | 0.560 | 0.626 | 0.172 | 1.49E−120 |
| MRPS35 | 1.53E−124 | 0.409 | 0.841 | 0.381 | 1.74E−120 |
| TLE1 | 2.29E−124 | 0.333 | 0.499 | 0.113 | 2.61E−120 |
| SUV420H1 | 4.21E−124 | 0.333 | 0.532 | 0.133 | 4.80E−120 |
| CHMP2B | 6.37E−124 | 0.394 | 0.687 | 0.241 | 7.26E−120 |
| SPINT2 | 7.97E−124 | 0.295 | 0.915 | 0.493 | 9.09E−120 |
| ATG12 | 1.30E−123 | 0.250 | 0.508 | 0.133 | 1.49E−119 |
| MRPL22 | 1.37E−123 | 0.312 | 0.607 | 0.195 | 1.56E−119 |
| MTPN | 1.44E−123 | 0.300 | 0.61 | 0.197 | 1.64E−119 |
| DHRS7 | 1.85E−123 | 0.312 | 0.705 | 0.262 | 2.11E−119 |
| UBB | 2.99E−123 | 0.553 | 0.974 | 0.611 | 3.41E−119 |
| PDIA3 | 3.24E−123 | 0.471 | 0.938 | 0.525 | 3.70E−119 |
| PRRC1 | 6.68E−123 | 0.395 | 0.527 | 0.129 | 7.62E−119 |
| DYNLL1 | 7.13E−123 | 0.263 | 0.844 | 0.409 | 8.13E−119 |
| CFL1 | 1.12E−122 | 0.466 | 0.964 | 0.573 | 1.28E−118 |
| HOXB6 | 1.51E−122 | 0.571 | 0.316 | 0.003 | 1.72E−118 |
| CD9 | 5.25E−122 | 0.399 | 0.918 | 0.486 | 5.98E−118 |
| ZNF217 | 7.54E−122 | 0.276 | 0.537 | 0.15 | 8.60E−118 |
| MAP7 | 8.18E−122 | 0.382 | 0.495 | 0.107 | 9.32E−118 |
| RPL34 | 8.67E−122 | 0.566 | 0.997 | 0.844 | 9.88E−118 |
| SERF2 | 1.28E−121 | 0.321 | 0.994 | 0.773 | 1.46E−117 |
| TCIRG1 | 1.39E−121 | 0.412 | 0.308 | 0.011 | 1.58E−117 |
| PTGES3 | 1.47E−121 | 0.311 | 0.835 | 0.402 | 1.67E−117 |
| CALM2 | 1.92E−121 | 0.457 | 0.981 | 0.671 | 2.18E−117 |
| PPP4C | 2.46E−121 | 0.438 | 0.586 | 0.166 | 2.81E−117 |
| MPDU1 | 2.70E−121 | 0.323 | 0.464 | 0.1 | 3.08E−117 |
| ELOVL5 | 2.70E−121 | 0.299 | 0.56 | 0.166 | 3.08E−117 |
| ETS2 | 3.07E−121 | 0.378 | 0.446 | 0.081 | 3.49E−117 |
| PDAP1 | 4.05E−121 | 0.398 | 0.597 | 0.173 | 4.62E−117 |
| TMEM205 | 7.40E−121 | 0.269 | 0.544 | 0.158 | 8.43E−117 |
| YPEL5 | 7.54E−121 | 0.283 | 0.555 | 0.16 | 8.59E−117 |
| SCAMP1 | 9.28E−121 | 0.255 | 0.505 | 0.138 | 1.06E−116 |
| WFDC2 | 1.07E−120 | 0.961 | 0.437 | 0.048 | 1.22E−116 |
| SRCAP | 1.23E−120 | 0.397 | 0.474 | 0.097 | 1.40E−116 |
| UBE2J1 | 1.47E−120 | 0.294 | 0.492 | 0.122 | 1.68E−116 |
| GATA3 | 1.72E−120 | 0.295 | 0.694 | 0.255 | 1.95E−116 |
| BRD2 | 2.02E−120 | 0.313 | 0.74 | 0.301 | 2.30E−116 |
| EIF1 | 2.35E−120 | 0.558 | 0.983 | 0.653 | 2.68E−116 |
| ETF1 | 4.13E−120 | 0.254 | 0.645 | 0.237 | 4.71E−116 |
| DNAJC15 | 4.28E−120 | 0.343 | 0.498 | 0.12 | 4.88E−116 |
| EMC3 | 8.06E−120 | 0.370 | 0.506 | 0.121 | 9.18E−116 |
| UGDH | 1.32E−119 | 0.531 | 0.61 | 0.168 | 1.50E−115 |
| NFKBIZ | 2.08E−119 | 0.462 | 0.467 | 0.086 | 2.37E−115 |
| PPFIA1 | 2.25E−119 | 0.293 | 0.698 | 0.265 | 2.56E−115 |
| CNDP2 | 3.16E−119 | 0.268 | 0.521 | 0.144 | 3.61E−115 |
| ANXA4 | 4.39E−119 | 0.296 | 0.458 | 0.106 | 5.01E−115 |
| FAM129A | 7.89E−119 | 0.438 | 0.476 | 0.092 | 8.99E−115 |
| FIS1 | 1.01E−118 | 0.404 | 0.548 | 0.144 | 1.16E−114 |
| ATP6V1F | 1.12E−118 | 0.333 | 0.584 | 0.18 | 1.28E−114 |
| AKR1A1 | 1.78E−118 | 0.307 | 0.564 | 0.17 | 2.03E−114 |
| LAPTM4A | 3.72E−118 | 0.301 | 0.793 | 0.358 | 4.24E−114 |
| GFPT1 | 4.02E−118 | 0.447 | 0.639 | 0.203 | 4.58E−114 |
| MRPL54 | 9.32E−118 | 0.303 | 0.479 | 0.116 | 1.06E−113 |
| EPHX2 | 1.26E−117 | 0.484 | 0.322 | 0.01 | 1.43E−113 |
| RNASET2 | 1.34E−117 | 0.311 | 0.407 | 0.072 | 1.53E−113 |
| TMEM167A | 2.06E−117 | 0.259 | 0.594 | 0.198 | 2.35E−113 |
| HSPA5 | 2.14E−117 | 0.260 | 0.935 | 0.535 | 2.43E−113 |
| NPNT | 3.72E−117 | 0.695 | 0.499 | 0.082 | 4.24E−113 |
| CIR1 | 4.01E−117 | 0.267 | 0.538 | 0.156 | 4.57E−113 |
| COQ10B | 4.17E−117 | 0.365 | 0.449 | 0.094 | 4.75E−113 |
| PPFIBP1 | 1.27E−116 | 0.377 | 0.513 | 0.124 | 1.45E−112 |
| LYPD3 | 1.42E−116 | 0.551 | 0.337 | 0.012 | 1.62E−112 |
| TRIP6 | 4.34E−116 | 0.352 | 0.416 | 0.075 | 4.95E−112 |
| SCAMP2 | 5.33E−116 | 0.448 | 0.43 | 0.067 | 6.08E−112 |
| SCOC | 5.63E−116 | 0.399 | 0.639 | 0.213 | 6.42E−112 |
| KIAA1324 | 5.93E−116 | 0.441 | 0.495 | 0.107 | 6.76E−112 |
| PFDN1 | 6.28E−116 | 0.379 | 0.625 | 0.201 | 7.15E−112 |
| PDE4DIP | 6.81E−116 | 0.254 | 0.414 | 0.085 | 7.76E−112 |
| LDLR | 8.68E−116 | 0.356 | 0.627 | 0.209 | 9.90E−112 |
| TUBA1B | 1.02E−115 | 0.618 | 0.875 | 0.43 | 1.16E−111 |
| MDM2 | 2.16E−115 | 0.588 | 0.565 | 0.14 | 2.46E−111 |
| SDC1 | 4.75E−115 | 0.601 | 0.565 | 0.136 | 5.42E−111 |
| ANKRD13D | 8.27E−115 | 0.510 | 0.423 | 0.058 | 9.42E−111 |
| EFCAB14 | 1.80E−114 | 0.416 | 0.543 | 0.144 | 2.05E−110 |
| IER5 | 2.06E−114 | 0.370 | 0.488 | 0.114 | 2.35E−110 |
| TTC1 | 4.33E−114 | 0.283 | 0.632 | 0.222 | 4.93E−110 |
| SMIM19 | 4.65E−114 | 0.344 | 0.382 | 0.056 | 5.30E−110 |
| FLII | 1.24E−113 | 0.279 | 0.459 | 0.107 | 1.41E−109 |
| ETV3 | 1.44E−113 | 0.293 | 0.455 | 0.105 | 1.64E−109 |
| FDPS | 1.75E−113 | 0.554 | 0.706 | 0.258 | 1.99E−109 |
| TSC22D3 | 2.07E−113 | 0.342 | 0.458 | 0.096 | 2.36E−109 |
| PPAPDC1B | 2.67E−113 | 0.404 | 0.53 | 0.14 | 3.04E−109 |
| RP11-294O2.2 | 3.97E−113 | 0.531 | 0.284 | 0.002 | 4.53E−109 |
| MPG | 4.78E−113 | 0.369 | 0.503 | 0.123 | 5.45E−109 |
| SLC39A8 | 9.64E−113 | 0.518 | 0.415 | 0.055 | 1.10E−108 |
| KIAA0040 | 2.33E−112 | 0.433 | 0.426 | 0.071 | 2.65E−108 |
| AIP | 3.96E−112 | 0.487 | 0.528 | 0.127 | 4.51E−108 |
| OAS1 | 7.19E−112 | 0.359 | 0.345 | 0.04 | 8.20E−108 |
| TP53TG1 | 7.57E−112 | 0.446 | 0.38 | 0.044 | 8.63E−108 |
| NANS | 7.59E−112 | 0.385 | 0.493 | 0.118 | 8.65E−108 |
| ACTG1 | 1.04E−111 | 0.974 | 0.978 | 0.791 | 1.19E−107 |
| CRABP2 | 1.12E−111 | 0.544 | 0.748 | 0.295 | 1.28E−107 |
| PNPLA4 | 1.72E−111 | 0.447 | 0.379 | 0.045 | 1.96E−107 |
| MYL9 | 2.12E−111 | 0.605 | 0.356 | 0.029 | 2.41E−107 |
| SCYL3 | 3.07E−111 | 0.441 | 0.37 | 0.041 | 3.50E−107 |
| C21orf59 | 4.23E−111 | 0.378 | 0.499 | 0.124 | 4.83E−107 |
| DHRSX | 1.33E−110 | 0.275 | 0.332 | 0.043 | 1.51E−106 |
| IK | 1.39E−110 | 0.326 | 0.582 | 0.183 | 1.59E−106 |
| MDH2 | 1.89E−110 | 0.282 | 0.699 | 0.289 | 2.16E−106 |
| ARPC4 | 2.34E−110 | 0.277 | 0.469 | 0.121 | 2.67E−106 |
| SH3BGRL | 2.60E−110 | 0.511 | 0.689 | 0.246 | 2.96E−106 |
| USMG5 | 3.51E−110 | 0.285 | 0.885 | 0.482 | 4.00E−106 |
| ANXA2 | 4.19E−110 | 0.250 | 0.9 | 0.501 | 4.77E−106 |
| C1orf56 | 1.57E−109 | 0.496 | 0.55 | 0.144 | 1.79E−105 |
| RAC1 | 2.18E−109 | 0.525 | 0.763 | 0.313 | 2.48E−105 |
| ORMDL2 | 2.39E−109 | 0.489 | 0.498 | 0.11 | 2.72E−105 |
| IRF7 | 2.66E−109 | 0.315 | 0.321 | 0.035 | 3.04E−105 |
| NPTN | 3.37E−109 | 0.317 | 0.481 | 0.125 | 3.84E−105 |
| OFD1 | 4.52E−109 | 0.326 | 0.443 | 0.095 | 5.15E−105 |
| NACA | 6.96E−109 | 0.403 | 0.955 | 0.586 | 7.94E−105 |
| RASEF | 1.12E−108 | 0.503 | 0.411 | 0.059 | 1.28E−104 |
| PPA2 | 2.25E−108 | 0.305 | 0.551 | 0.17 | 2.57E−104 |
| CACNB3 | 2.65E−108 | 0.482 | 0.293 | 0.005 | 3.02E−104 |
| MEAF6 | 5.58E−108 | 0.270 | 0.555 | 0.183 | 6.36E−104 |
| SFT2D1 | 1.23E−107 | 0.301 | 0.496 | 0.132 | 1.40E−103 |
| FAM188A | 2.21E−107 | 0.381 | 0.279 | 0.008 | 2.52E−103 |
| MRPL11 | 2.86E−107 | 0.370 | 0.572 | 0.176 | 3.27E−103 |
| KIF13B | 5.21E−107 | 0.475 | 0.455 | 0.089 | 5.94E−103 |
| ATF3 | 5.37E−107 | 0.659 | 0.632 | 0.194 | 6.12E−103 |
| BLOC1S2 | 6.51E−107 | 0.361 | 0.446 | 0.097 | 7.42E−103 |
| CMYA5 | 2.08E−106 | 0.378 | 0.352 | 0.046 | 2.37E−102 |
| RASSF3 | 2.12E−106 | 0.475 | 0.382 | 0.052 | 2.42E−102 |
| RASD1 | 2.34E−106 | 0.494 | 0.508 | 0.116 | 2.67E−102 |
| MBNL2 | 5.98E−106 | 0.369 | 0.589 | 0.188 | 6.82E−102 |
| CTC-425F1.4 | 6.95E−106 | 0.621 | 0.616 | 0.183 | 7.92E−102 |
| HSP90AB1 | 3.61E−105 | 0.296 | 0.998 | 0.804 | 4.11E−101 |
| DAPP1 | 3.61E−105 | 0.260 | 0.304 | 0.036 | 4.11E−101 |
| STX12 | 5.38E−105 | 0.429 | 0.408 | 0.069 | 6.13E−101 |
| SDF4 | 7.49E−105 | 0.280 | 0.464 | 0.125 | 8.53E−101 |
| NR4A1 | 1.03E−104 | 0.410 | 0.621 | 0.208 | 1.17E−100 |
| METTL9 | 1.34E−104 | 0.278 | 0.522 | 0.159 | 1.52E−100 |
| VAMP3 | 1.59E−104 | 0.418 | 0.402 | 0.066 | 1.82E−100 |
| RARRES3 | 1.85E−104 | 0.284 | 0.394 | 0.081 | 2.11E−100 |
| BBC3 | 4.32E−104 | 0.662 | 0.401 | 0.048 | 4.93E−100 |
| C1orf63 | 1.78E−103 | 0.393 | 0.464 | 0.107 | 2.03E−99 |
| RTF1 | 2.57E−103 | 0.312 | 0.554 | 0.175 | 2.93E−99 |
| CCDC125 | 3.89E−103 | 0.281 | 0.411 | 0.093 | 4.43E−99 |
| CYSTM1 | 1.03E−102 | 0.358 | 0.441 | 0.098 | 1.17E−98 |
| PIH1D1 | 1.32E−102 | 0.414 | 0.453 | 0.098 | 1.50E−98 |
| ZFP36 | 1.68E−102 | 0.288 | 0.715 | 0.306 | 1.92E−98 |
| BOLA3 | 1.86E−102 | 0.265 | 0.49 | 0.146 | 2.12E−98 |
| RPLP2 | 2.01E−102 | 0.317 | 0.999 | 0.888 | 2.29E−98 |
| ETFA | 2.47E−102 | 0.385 | 0.563 | 0.175 | 2.82E−98 |
| GDE1 | 2.74E−102 | 0.464 | 0.492 | 0.12 | 3.12E−98 |
| AP1M2 | 6.59E−102 | 0.368 | 0.336 | 0.039 | 7.51E−98 |
| ALG8 | 7.22E−102 | 0.520 | 0.534 | 0.136 | 8.23E−98 |
| YIPF5 | 7.34E−102 | 0.341 | 0.506 | 0.142 | 8.37E−98 |
| IFNGR2 | 8.19E−102 | 0.263 | 0.339 | 0.058 | 9.33E−98 |
| ARL8B | 5.73E−101 | 0.263 | 0.499 | 0.153 | 6.53E−97 |
| CTC-228N24.3 | 1.25E−100 | 0.251 | 0.295 | 0.036 | 1.43E−96 |
| RMDN3 | 1.77E−100 | 0.340 | 0.342 | 0.046 | 2.01E−96 |
| EFHD2 | 1.92E−100 | 0.317 | 0.314 | 0.037 | 2.19E−96 |
| SRI | 4.54E−100 | 0.604 | 0.547 | 0.145 | 5.18E−96 |
| THUMPD3 | 6.15E−100 | 0.274 | 0.445 | 0.119 | 7.01E−96 |
| FRMD4B | 8.04E−100 | 0.371 | 0.387 | 0.071 | 9.16E−96 |
| SF3B4 | 8.45E−100 | 0.294 | 0.379 | 0.075 | 9.63E−96 |
| WBSCR22 | 8.95E−100 | 0.284 | 0.605 | 0.227 | 1.02E−95 |
| PRR15 | 9.63E−100 | 0.504 | 0.345 | 0.03 | 1.10E−95 |
| PRKRIP1 | 1.14E−99 | 0.292 | 0.412 | 0.088 | 1.30E−95 |
| MICALL2 | 2.26E−99 | 0.392 | 0.302 | 0.022 | 2.58E−95 |
| CALCOCO2 | 2.51E−99 | 0.287 | 0.475 | 0.134 | 2.86E−95 |
| NTHL1 | 3.67E−99 | 0.528 | 0.374 | 0.044 | 4.18E−95 |
| C4orf32 | 4.52E−99 | 0.547 | 0.332 | 0.023 | 5.15E−95 |
| SNAP23 | 4.82E−99 | 0.347 | 0.45 | 0.11 | 5.49E−95 |
| TSPAN15 | 5.80E−99 | 0.470 | 0.511 | 0.129 | 6.61E−95 |
| CAMSAP2 | 6.92E−99 | 0.391 | 0.44 | 0.099 | 7.89E−95 |
| SRD5A3 | 7.18E−99 | 0.364 | 0.412 | 0.082 | 8.18E−95 |
| SPINT1 | 7.49E−99 | 0.432 | 0.405 | 0.069 | 8.54E−95 |
| CTD-2015H6.3 | 1.23E−98 | 0.412 | 0.384 | 0.062 | 1.40E−94 |
| KRT10 | 2.68E−98 | 0.437 | 0.546 | 0.159 | 3.05E−94 |
| HOXC10 | 2.77E−98 | 0.472 | 0.37 | 0.046 | 3.16E−94 |
| SPOPL | 2.94E−98 | 0.257 | 0.408 | 0.096 | 3.35E−94 |
| FAM214A | 5.93E−98 | 0.453 | 0.396 | 0.064 | 6.76E−94 |
| MANSC1 | 1.39E−97 | 0.551 | 0.357 | 0.035 | 1.58E−93 |
| CYB5R1 | 3.15E−97 | 0.435 | 0.336 | 0.034 | 3.60E−93 |
| KLF2 | 4.54E−97 | 0.382 | 0.338 | 0.043 | 5.17E−93 |
| GINM1 | 4.76E−97 | 0.341 | 0.484 | 0.134 | 5.43E−93 |
| PVRL2 | 9.29E−97 | 0.330 | 0.318 | 0.04 | 1.06E−92 |
| NT5C3A | 4.48E−96 | 0.360 | 0.531 | 0.163 | 5.11E−92 |
| DGCR6L | 6.91E−96 | 0.360 | 0.411 | 0.087 | 7.88E−92 |
| SREK1IP1 | 1.34E−95 | 0.335 | 0.441 | 0.111 | 1.53E−91 |
| CCS | 1.34E−95 | 0.464 | 0.362 | 0.046 | 1.53E−91 |
| NFATC4 | 1.60E−95 | 0.451 | 0.267 | 0.005 | 1.82E−91 |
| COBL | 1.68E−95 | 0.284 | 0.394 | 0.088 | 1.91E−91 |
| PPCS | 2.12E−95 | 0.276 | 0.51 | 0.162 | 2.42E−91 |
| ACVR2A | 4.17E−95 | 0.307 | 0.336 | 0.052 | 4.75E−91 |
| GTF2H5 | 6.13E−95 | 0.275 | 0.466 | 0.135 | 6.99E−91 |
| RPS15 | 6.54E−95 | 0.285 | 0.984 | 0.687 | 7.45E−91 |
| ALDH3B1 | 1.92E−94 | 0.430 | 0.287 | 0.014 | 2.19E−90 |
| SLC37A1 | 2.08E−94 | 0.418 | 0.312 | 0.025 | 2.37E−90 |
| ASB8 | 2.37E−94 | 0.385 | 0.358 | 0.056 | 2.70E−90 |
| CLTB | 6.70E−94 | 0.341 | 0.401 | 0.087 | 7.64E−90 |
| CTBP2 | 1.10E−93 | 0.337 | 0.55 | 0.183 | 1.25E−89 |
| SIL1 | 1.51E−93 | 0.342 | 0.401 | 0.085 | 1.72E−89 |
| SIPA1L2 | 3.12E−93 | 0.326 | 0.256 | 0.016 | 3.56E−89 |
| DSG2 | 4.60E−93 | 0.330 | 0.363 | 0.066 | 5.24E−89 |
| RPL23 | 6.20E−93 | 0.269 | 0.982 | 0.699 | 7.07E−89 |
| CA2 | 7.35E−93 | 0.646 | 0.332 | 0.032 | 8.38E−89 |
| LAD1 | 8.43E−93 | 0.269 | 0.285 | 0.034 | 9.61E−89 |
| BECN1 | 8.46E−93 | 0.280 | 0.397 | 0.091 | 9.64E−89 |
| MISP | 8.56E−93 | 0.376 | 0.243 | 0.002 | 9.76E−89 |
| MAST4 | 4.62E−92 | 0.472 | 0.338 | 0.037 | 5.27E−88 |
| GBP1 | 1.17E−91 | 0.284 | 0.427 | 0.115 | 1.34E−87 |
| MYADM | 1.87E−91 | 0.372 | 0.513 | 0.154 | 2.13E−87 |
| RPA3 | 1.97E−91 | 0.379 | 0.533 | 0.167 | 2.25E−87 |
| POLB | 3.14E−91 | 0.316 | 0.373 | 0.075 | 3.58E−87 |
| CDR2 | 3.42E−91 | 0.411 | 0.346 | 0.049 | 3.90E−87 |
| C12orf44 | 9.65E−91 | 0.476 | 0.429 | 0.09 | 1.10E−86 |
| ERAP1 | 1.87E−90 | 0.320 | 0.304 | 0.037 | 2.13E−86 |
| PPIC | 5.12E−90 | 0.387 | 0.32 | 0.038 | 5.83E−86 |
| FUCA1 | 6.33E−90 | 0.346 | 0.273 | 0.021 | 7.21E−86 |
| NAT1 | 9.67E−90 | 0.316 | 0.236 | 0.01 | 1.10E−85 |
| PRAF2 | 1.17E−89 | 0.359 | 0.337 | 0.049 | 1.33E−85 |
| RANBP1 | 1.78E−89 | 0.329 | 0.704 | 0.312 | 2.03E−85 |
| PAQR4 | 2.24E−89 | 0.394 | 0.316 | 0.038 | 2.55E−85 |
| MYLIP | 2.72E−89 | 0.328 | 0.407 | 0.095 | 3.10E−85 |
| MAP3K8 | 3.28E−89 | 0.269 | 0.268 | 0.031 | 3.73E−85 |
| TNIK | 3.60E−89 | 0.334 | 0.323 | 0.05 | 4.10E−85 |
| TMEM150C | 3.63E−89 | 0.419 | 0.239 | 0.003 | 4.13E−85 |
| ZFYVE21 | 4.25E−89 | 0.313 | 0.381 | 0.082 | 4.84E−85 |
| SELM | 4.59E−89 | 0.362 | 0.447 | 0.119 | 5.23E−85 |
| LIPH | 9.82E−89 | 0.312 | 0.293 | 0.037 | 1.12E−84 |
| RUSC1 | 1.28E−88 | 0.342 | 0.388 | 0.084 | 1.46E−84 |
| STK39 | 1.29E−88 | 0.359 | 0.305 | 0.034 | 1.47E−84 |
| FOXJ3 | 1.33E−88 | 0.293 | 0.392 | 0.094 | 1.51E−84 |
| MESDC2 | 1.36E−88 | 0.294 | 0.477 | 0.145 | 1.55E−84 |
| CCNG1 | 1.55E−88 | 0.284 | 0.466 | 0.141 | 1.77E−84 |
| USP53 | 2.09E−88 | 0.357 | 0.503 | 0.155 | 2.38E−84 |
| SPTSSB | 2.17E−88 | 0.499 | 0.272 | 0.013 | 2.47E−84 |
| FAM120AOS | 2.67E−88 | 0.272 | 0.46 | 0.141 | 3.05E−84 |
| FUNDC1 | 4.59E−88 | 0.273 | 0.302 | 0.044 | 5.24E−84 |
| KRTCAP3 | 8.67E−88 | 0.388 | 0.343 | 0.049 | 9.89E−84 |
| CTBS | 1.14E−87 | 0.294 | 0.319 | 0.05 | 1.30E−83 |
| ST6GALNAC2 | 2.02E−87 | 0.417 | 0.306 | 0.03 | 2.31E−83 |
| GATA2 | 4.40E−87 | 0.456 | 0.305 | 0.027 | 5.02E−83 |
| MFSD2A | 7.90E−87 | 0.301 | 0.279 | 0.031 | 9.00E−83 |
| ALG5 | 2.50E−86 | 0.290 | 0.41 | 0.106 | 2.85E−82 |
| COMMD3 | 3.47E−86 | 0.271 | 0.469 | 0.145 | 3.96E−82 |
| CISD3 | 6.16E−86 | 0.342 | 0.396 | 0.09 | 7.03E−82 |
| SH2D4A | 6.27E−86 | 0.333 | 0.301 | 0.039 | 7.14E−82 |
| PARM1 | 1.00E−85 | 0.470 | 0.272 | 0.017 | 1.14E−81 |
| LCOR | 1.65E−85 | 0.266 | 0.356 | 0.078 | 1.88E−81 |
| FBXL5 | 3.25E−85 | 0.276 | 0.412 | 0.113 | 3.70E−81 |
| SMAD5 | 3.73E−85 | 0.327 | 0.483 | 0.147 | 4.25E−81 |
| SH3BGRL2 | 3.99E−85 | 0.339 | 0.231 | 0.007 | 4.55E−81 |
| ARL15 | 4.04E−85 | 0.356 | 0.238 | 0.008 | 4.60E−81 |
| DMKN | 4.83E−85 | 0.306 | 0.293 | 0.039 | 5.51E−81 |
| ZYX | 6.48E−85 | 0.318 | 0.39 | 0.09 | 7.38E−81 |
| TJP3 | 7.55E−85 | 0.400 | 0.277 | 0.021 | 8.61E−81 |
| MAPK9 | 9.61E−85 | 0.341 | 0.356 | 0.071 | 1.10E−80 |
| ZFAND6 | 3.55E−84 | 0.268 | 0.454 | 0.138 | 4.04E−80 |
| TMPRSS3 | 7.25E−84 | 0.332 | 0.23 | 0.005 | 8.26E−80 |
| F2RL1 | 4.95E−83 | 0.445 | 0.261 | 0.014 | 5.64E−79 |
| ANG | 1.17E−82 | 0.357 | 0.237 | 0.009 | 1.33E−78 |
| DOPEY2 | 2.05E−82 | 0.375 | 0.333 | 0.055 | 2.33E−78 |
| VASP | 5.40E−82 | 0.252 | 0.359 | 0.087 | 6.16E−78 |
| HAGH | 6.40E−82 | 0.304 | 0.327 | 0.062 | 7.30E−78 |
| MIR22HG | 1.14E−81 | 0.395 | 0.272 | 0.024 | 1.30E−77 |
| TTC9 | 1.47E−81 | 0.404 | 0.333 | 0.052 | 1.68E−77 |
| RHBDD2 | 4.53E−81 | 0.332 | 0.407 | 0.106 | 5.16E−77 |
| PLEKHA1 | 1.77E−80 | 0.254 | 0.396 | 0.109 | 2.01E−76 |
| ECU | 2.22E−80 | 0.391 | 0.453 | 0.126 | 2.53E−76 |
| CXXC5 | 2.29E−80 | 0.406 | 0.398 | 0.087 | 2.61E−76 |
| ERAP2 | 3.25E−80 | 0.364 | 0.246 | 0.015 | 3.71E−76 |
| VCL | 7.85E−80 | 0.590 | 0.638 | 0.251 | 8.95E−76 |
| FADS2 | 1.17E−79 | 0.261 | 0.681 | 0.322 | 1.34E−75 |
| HEBP1 | 2.20E−79 | 0.253 | 0.377 | 0.1 | 2.51E−75 |
| SYTL1 | 2.51E−79 | 0.405 | 0.277 | 0.024 | 2.86E−75 |
| PAFAH1B3 | 3.24E−79 | 0.279 | 0.458 | 0.148 | 3.69E−75 |
| CRB3 | 1.05E−78 | 0.372 | 0.293 | 0.033 | 1.20E−74 |
| CORO1B | 1.40E−78 | 0.371 | 0.288 | 0.03 | 1.59E−74 |
| FAM173A | 4.81E−78 | 0.388 | 0.345 | 0.062 | 5.49E−74 |
| PARVA | 4.94E−78 | 0.255 | 0.257 | 0.032 | 5.63E−74 |
| FAM174A | 4.04E−77 | 0.389 | 0.328 | 0.053 | 4.61E−73 |
| PYCR1 | 5.53E−77 | 0.263 | 0.429 | 0.132 | 6.31E−73 |
| DDX23 | 6.65E−76 | 0.278 | 0.324 | 0.065 | 7.58E−72 |
| PIK3R3 | 1.06E−75 | 0.320 | 0.469 | 0.153 | 1.21E−71 |
| C1orf115 | 1.88E−75 | 0.405 | 0.29 | 0.031 | 2.14E−71 |
| PDZD8 | 2.06E−75 | 0.287 | 0.325 | 0.07 | 2.35E−71 |
| CNTNAP2 | 2.74E−75 | 0.505 | 0.411 | 0.094 | 3.13E−71 |
| RABL5 | 8.25E−75 | 0.291 | 0.333 | 0.071 | 9.40E−71 |
| SPSB3 | 2.27E−74 | 0.270 | 0.298 | 0.053 | 2.59E−70 |
| TUBB2A | 3.12E−74 | 0.345 | 0.317 | 0.056 | 3.55E−70 |
| FADD | 6.77E−74 | 0.261 | 0.404 | 0.12 | 7.72E−70 |
| RPS23 | 1.05E−73 | 0.252 | 0.995 | 0.8 | 1.19E−69 |
| SPR | 1.22E−72 | 0.440 | 0.344 | 0.059 | 1.39E−68 |
| VASN | 1.61E−72 | 0.326 | 0.224 | 0.012 | 1.84E−68 |
| DNAJC22 | 2.66E−72 | 0.383 | 0.266 | 0.025 | 3.04E−68 |
| ZNF365 | 6.15E−72 | 0.360 | 0.291 | 0.045 | 7.01E−68 |
| CEACAM6 | 6.58E−72 | 0.638 | 0.233 | 0.011 | 7.50E−68 |
| GATM | 9.14E−72 | 0.259 | 0.242 | 0.03 | 1.04E−67 |
| PHTF2 | 9.51E−71 | 0.267 | 0.307 | 0.065 | 1.08E−66 |
| DNALI1 | 1.32E−70 | 0.367 | 0.181 | 0 | 1.51E−66 |
| MSRB1 | 3.25E−70 | 0.262 | 0.247 | 0.033 | 3.71E−66 |
| CLDN12 | 2.20E−69 | 0.286 | 0.255 | 0.035 | 2.51E−65 |
| VANGL1 | 3.16E−69 | 0.355 | 0.297 | 0.049 | 3.60E−65 |
| PYROXD1 | 1.02E−68 | 0.272 | 0.324 | 0.073 | 1.16E−64 |
| PLS1 | 1.08E−68 | 0.275 | 0.219 | 0.021 | 1.23E−64 |
| SCD | 1.81E−68 | 0.594 | 0.825 | 0.48 | 2.06E−64 |
| ZC3H12A | 6.15E−68 | 0.332 | 0.239 | 0.019 | 7.01E−64 |
| BMP4 | 2.34E−67 | 0.300 | 0.173 | 0 | 2.67E−63 |
| GPRC5C | 2.63E−67 | 0.311 | 0.272 | 0.037 | 3.00E−63 |
| BAG3 | 3.96E−67 | 0.346 | 0.303 | 0.053 | 4.52E−63 |
| DCTD | 2.04E−66 | 0.271 | 0.304 | 0.065 | 2.33E−62 |
| ZDHHC24 | 2.20E−66 | 0.269 | 0.277 | 0.052 | 2.50E−62 |
| ACADSB | 3.07E−66 | 0.349 | 0.288 | 0.045 | 3.50E−62 |
| CLASRP | 9.70E−66 | 0.274 | 0.317 | 0.075 | 1.11E−61 |
| ATHL1 | 1.84E−65 | 0.303 | 0.195 | 0.01 | 2.10E−61 |
| MGLL | 2.55E−65 | 0.312 | 0.258 | 0.038 | 2.91E−61 |
| CRAT | 4.69E−65 | 0.261 | 0.337 | 0.086 | 5.35E−61 |
| SERTAD3 | 6.44E−64 | 0.281 | 0.222 | 0.02 | 7.34E−60 |
| S100A6 | 1.02E−63 | 0.505 | 0.923 | 0.638 | 1.16E−59 |
| PPP1R35 | 1.38E−63 | 0.386 | 0.348 | 0.077 | 1.57E−59 |
| TRIM16 | 3.09E−63 | 0.275 | 0.225 | 0.026 | 3.52E−59 |
| ST8SIA6-AS1 | 6.40E−63 | 0.271 | 0.169 | 0.001 | 7.29E−59 |
| JAGN1 | 7.44E−63 | 0.299 | 0.309 | 0.071 | 8.48E−59 |
| DMBT1 | 9.45E−63 | 0.640 | 0.167 | 0.001 | 1.08E−58 |
| NUDT12 | 8.53E−62 | 0.283 | 0.216 | 0.018 | 9.72E−58 |
| CCDC170 | 1.21E−61 | 0.277 | 0.172 | 0.005 | 1.38E−57 |
| MMEL1 | 2.40E−61 | 0.267 | 0.206 | 0.017 | 2.74E−57 |
| RAB27B | 3.87E−61 | 0.259 | 0.359 | 0.106 | 4.41E−57 |
| KRT81 | 6.14E−61 | 1.023 | 0.206 | 0.015 | 7.00E−57 |
| PITPNM1 | 6.94E−61 | 0.251 | 0.202 | 0.02 | 7.91E−57 |
| PMAIP1 | 1.18E−60 | 0.270 | 0.314 | 0.08 | 1.34E−56 |
| PART1 | 1.85E−60 | 0.385 | 0.23 | 0.021 | 2.11E−56 |
| ABCA12 | 3.09E−59 | 0.322 | 0.217 | 0.017 | 3.52E−55 |
| SCNN1A | 5.55E−59 | 0.339 | 0.248 | 0.032 | 6.32E−55 |
| RNF121 | 7.13E−59 | 0.263 | 0.21 | 0.021 | 8.13E−55 |
| DNAJA4 | 3.71E−58 | 0.260 | 0.301 | 0.075 | 4.22E−54 |
| TNFRSF10B | 6.69E−58 | 0.259 | 0.229 | 0.033 | 7.62E−54 |
| HOXB7 | 1.26E−57 | 0.430 | 0.289 | 0.049 | 1.44E−53 |
| RAD9A | 1.53E−56 | 0.263 | 0.237 | 0.037 | 1.74E−52 |
| KIAA1377 | 1.53E−56 | 0.289 | 0.183 | 0.014 | 1.74E−52 |
| IL4R | 2.05E−56 | 0.280 | 0.198 | 0.017 | 2.34E−52 |
| CEACAM5 | 4.00E−56 | 0.501 | 0.157 | 0.003 | 4.56E−52 |
| PRSS22 | 7.05E−56 | 0.275 | 0.194 | 0.015 | 8.04E−52 |
| GRIP1 | 1.83E−55 | 0.268 | 0.182 | 0.014 | 2.08E−51 |
| RGS16 | 2.41E−55 | 0.375 | 0.199 | 0.014 | 2.75E−51 |
| SEMA3E | 8.94E−55 | 0.296 | 0.19 | 0.015 | 1.02E−50 |
| MAP9 | 9.20E−54 | 0.255 | 0.357 | 0.115 | 1.05E−49 |
| EPS8L1 | 1.63E−52 | 0.332 | 0.256 | 0.043 | 1.86E−48 |
| TMEM45B | 4.27E−52 | 0.297 | 0.209 | 0.021 | 4.86E−48 |
| CYP3A5 | 8.26E−52 | 0.366 | 0.135 | 0 | 9.41E−48 |
| PKP3 | 1.32E−51 | 0.296 | 0.237 | 0.035 | 1.50E−47 |
| ZBTB42 | 1.92E−51 | 0.256 | 0.181 | 0.014 | 2.19E−47 |
| ACY3 | 4.98E−51 | 0.592 | 0.195 | 0.016 | 5.68E−47 |
| GADD45G | 8.23E−51 | 0.309 | 0.172 | 0.009 | 9.38E−47 |
| RP11-841020.2 | 8.58E−51 | 0.252 | 0.193 | 0.023 | 9.78E−47 |
| SLC9A3R2 | 1.28E−50 | 0.274 | 0.195 | 0.019 | 1.45E−46 |
| RHOV | 5.58E−50 | 0.297 | 0.183 | 0.014 | 6.36E−46 |
| TM4SF1 | 5.67E−50 | 0.253 | 0.614 | 0.338 | 6.46E−46 |
| LMTK3 | 1.83E−49 | 0.329 | 0.211 | 0.024 | 2.09E−45 |
| MT-TG | 8.23E−49 | 0.265 | 0.195 | 0.025 | 9.39E−45 |
| UNC93B1 | 8.49E−44 | 0.254 | 0.179 | 0.02 | 9.67E−40 |
| BCAS4 | 2.15E−41 | 0.258 | 0.222 | 0.046 | 2.45E−37 |
| NCAM2 | 2.26E−41 | 0.329 | 0.171 | 0.019 | 2.57E−37 |
| TNFAIP2 | 2.87E−41 | 0.539 | 0.218 | 0.039 | 3.27E−37 |
| IGHMBP2 | 5.81E−39 | 0.259 | 0.203 | 0.036 | 6.63E−35 |
| UBE2T | 1.31E−30 | 0.300 | 0.257 | 0.085 | 1.49E−26 |
| KIAA0101 | 9.36E−19 | 0.365 | 0.267 | 0.117 | 1.07E−14 |
| NUSAP1 | 1.12E−10 | 0.261 | 0.194 | 0.095 | 1.28E−06 |
| RP13-895J2.7 | 2.37E−10 | 0.994 | 0.133 | 0.063 | 2.70E−06 |

TABLE 9
CSF.f.mac.set.BMvLMD.markers

| | | | | |
|---|---|---|---|---|
| p_val | avg_logFC | pct.1 | pct.2 | p_val_adj |

CSF.f.mac.set.BMvLMD.markers.dn.1

| | | | | | |
|---|---|---|---|---|---|
| PSAP | 2.39E−38 | −1.178 | 0.792 | 0.939 | 1.92E−34 |
| IFIT2 | 6.31E−36 | −2.019 | 0.091 | 0.422 | 5.07E−32 |
| SPP1 | 9.49E−35 | −3.581 | 0.11 | 0.506 | 7.62E−31 |
| STAT1 | 3.46E−34 | −1.515 | 0.136 | 0.514 | 2.77E−30 |
| IFI44L | 1.75E−33 | −1.471 | 0.125 | 0.483 | 1.40E−29 |
| MX1 | 1.69E−32 | −1.577 | 0.144 | 0.492 | 1.35E−28 |
| IFI6 | 1.70E−32 | −1.662 | 0.167 | 0.538 | 1.37E−28 |
| IFIT3 | 1.83E−32 | −1.919 | 0.087 | 0.423 | 1.47E−28 |
| LAP3 | 3.96E−31 | −1.210 | 0.235 | 0.563 | 3.18E−27 |
| IFIT1 | 2.95E−30 | −1.819 | 0.042 | 0.377 | 2.37E−26 |
| CYBB | 3.22E−30 | −1.138 | 0.504 | 0.775 | 2.58E−26 |
| ISG15 | 3.71E−28 | −1.553 | 0.189 | 0.494 | 2.97E−24 |
| CTSS | 6.30E−28 | −1.016 | 0.769 | 0.894 | 5.06E−24 |
| C1QB | 1.72E−27 | −1.865 | 0.208 | 0.529 | 1.38E−23 |
| MSR1 | 4.12E−26 | −1.331 | 0.216 | 0.543 | 3.31E−22 |
| TLN1 | 5.98E−26 | −0.979 | 0.515 | 0.712 | 4.79E−22 |
| OAS2 | 1.75E−25 | −1.045 | 0.034 | 0.313 | 1.40E−21 |
| EPSTI1 | 2.03E−25 | −1.244 | 0.159 | 0.431 | 1.63E−21 |
| RSAD2 | 2.30E−25 | −1.389 | 0.03 | 0.298 | 1.85E−21 |
| IFI30 | 3.50E−25 | −1.019 | 0.561 | 0.79 | 2.81E−21 |
| TNFSF10 | 1.17E−24 | −1.636 | 0.125 | 0.376 | 9.40E−21 |
| MX2 | 3.43E−24 | −1.132 | 0.117 | 0.434 | 2.75E−20 |
| SAT1 | 4.78E−24 | −1.009 | 0.61 | 0.793 | 3.83E−20 |
| APOE | 8.99E−24 | −1.939 | 0.227 | 0.494 | 7.21E−20 |
| FCGR3A | 2.81E−23 | −1.133 | 0.186 | 0.529 | 2.26E−19 |
| GIMAP4 | 5.97E−23 | −1.121 | 0.155 | 0.405 | 4.79E−19 |
| RNF213 | 1.20E−22 | −1.075 | 0.292 | 0.518 | 9.60E−19 |
| RPS27 | 1.45E−22 | −0.494 | 0.769 | 0.64 | 1.17E−18 |
| HLA-A | 1.86E−22 | −0.746 | 0.739 | 0.871 | 1.49E−18 |
| APOL6 | 6.78E−22 | −0.939 | 0.129 | 0.387 | 5.44E−18 |
| CD68 | 1.16E−21 | −0.954 | 0.307 | 0.61 | 9.31E−18 |
| HERC5 | 2.21E−21 | −1.105 | 0.034 | 0.279 | 1.78E−17 |
| CD63 | 2.42E−21 | −0.950 | 0.371 | 0.644 | 1.94E−17 |
| HLA-B | 5.83E−21 | −0.602 | 0.932 | 0.962 | 4.68E−17 |
| MAFB | 7.69E−21 | −1.282 | 0.178 | 0.44 | 6.17E−17 |
| IFI16 | 7.71E−21 | −0.857 | 0.405 | 0.643 | 6.19E−17 |
| SAMD9L | 1.25E−20 | −1.010 | 0.133 | 0.434 | 1.01E−16 |
| SLA | 1.51E−20 | −0.960 | 0.14 | 0.4 | 1.21E−16 |
| IFITM3 | 2.89E−20 | −1.014 | 0.33 | 0.552 | 2.32E−16 |
| B2M | 4.33E−20 | −0.485 | 1 | 0.998 | 3.47E−16 |
| MTRNR2L12 | 4.89E−20 | −0.893 | 0.117 | 0.376 | 3.92E−16 |
| ITM2B | 5.42E−20 | −0.711 | 0.689 | 0.715 | 4.35E−16 |
| SAMD9 | 6.59E−20 | −0.987 | 0.167 | 0.402 | 5.28E−16 |
| FTL | 7.95E−20 | −0.899 | 0.973 | 0.974 | 6.38E−16 |
| C1QC | 8.01E−20 | −1.495 | 0.201 | 0.448 | 6.43E−16 |
| MS4A7 | 1.05E−19 | −1.052 | 0.201 | 0.44 | 8.41E−16 |
| PLSCR1 | 2.41E−19 | −0.901 | 0.201 | 0.425 | 1.93E−15 |
| CD163 | 2.87E−19 | −1.136 | 0.152 | 0.413 | 2.30E−15 |
| MARCKS | 3.70E−19 | −0.993 | 0.239 | 0.521 | 2.97E−15 |
| IFIH1 | 6.08E−19 | −1.072 | 0.14 | 0.36 | 4.88E−15 |
| EIF2AK2 | 1.16E−18 | −0.891 | 0.167 | 0.445 | 9.31E−15 |
| ARPC5 | 1.60E−18 | −0.724 | 0.617 | 0.791 | 1.28E−14 |
| SRGN | 2.48E−18 | −0.783 | 0.939 | 0.914 | 1.99E−14 |
| HSPA5 | 7.52E−18 | −0.856 | 0.277 | 0.374 | 6.03E−14 |
| OAS3 | 3.01E−17 | −0.795 | 0.03 | 0.25 | 2.41E−13 |
| CALR | 8.14E−17 | −0.776 | 0.379 | 0.584 | 6.53E−13 |
| CTSB | 9.33E−17 | −0.846 | 0.61 | 0.802 | 7.48E−13 |
| APLP2 | 1.07E−16 | −0.826 | 0.443 | 0.62 | 8.62E−13 |
| DDX58 | 1.23E−16 | −0.656 | 0.023 | 0.21 | 9.86E−13 |
| IFI44 | 1.55E−16 | −0.886 | 0.064 | 0.299 | 1.24E−12 |
| VCAN | 2.42E−16 | −2.463 | 0.155 | 0.362 | 1.94E−12 |
| TXNIP | 6.03E−16 | −0.802 | 0.697 | 0.791 | 4.84E−12 |
| NT5C3A | 6.85E−16 | −0.889 | 0.098 | 0.301 | 5.50E−12 |
| HLA-E | 8.78E−16 | −0.590 | 0.852 | 0.903 | 7.04E−12 |
| ENO1 | 1.86E−15 | −0.708 | 0.455 | 0.618 | 1.49E−11 |
| PLXDC2 | 3.96E−15 | −0.868 | 0.299 | 0.483 | 3.18E−11 |
| CYFIP1 | 1.01E−14 | −0.769 | 0.098 | 0.313 | 8.09E−11 |
| GBP1 | 1.11E−14 | −0.887 | 0.091 | 0.313 | 8.93E−11 |
| OAS1 | 1.79E−14 | −0.744 | 0.125 | 0.333 | 1.44E−10 |
| GPNMB | 1.81E−14 | −1.106 | 0.057 | 0.276 | 1.45E−10 |
| LAMP2 | 4.59E−14 | −0.697 | 0.197 | 0.391 | 3.69E−10 |
| XAF1 | 7.87E−14 | −0.815 | 0.36 | 0.549 | 6.31E−10 |
| RGS1 | 1.13E−13 | −0.694 | 0.496 | 0.459 | 9.09E−10 |
| ASAH1 | 1.47E−13 | −0.687 | 0.337 | 0.482 | 1.18E−09 |
| FGL2 | 1.54E−13 | −0.791 | 0.591 | 0.669 | 1.24E−09 |
| HIF1A | 1.57E−13 | −0.834 | 0.28 | 0.463 | 1.26E−09 |
| PYGL | 2.40E−13 | −0.567 | 0.011 | 0.17 | 1.93E−09 |
| PARP9 | 4.19E−13 | −0.808 | 0.106 | 0.327 | 3.36E−09 |
| FPR1 | 4.31E−13 | −0.728 | 0.208 | 0.406 | 3.46E−09 |
| LYZ | 4.42E−13 | −0.782 | 0.625 | 0.804 | 3.55E−09 |
| CYBA | 5.93E−13 | −0.525 | 0.356 | 0.537 | 4.76E−09 |
| BST2 | 7.02E−13 | −0.639 | 0.265 | 0.419 | 5.63E−09 |
| MTRNR2L2 | 9.52E−13 | −0.697 | 0.254 | 0.377 | 7.64E−09 |
| C1QA | 1.03E−12 | −0.969 | 0.193 | 0.442 | 8.29E−09 |
| RNA28S5 | 1.31E−12 | −0.450 | 0.841 | 0.775 | 1.05E−08 |
| SDCBP | 1.61E−12 | −0.678 | 0.208 | 0.367 | 1.29E−08 |
| GIMAP8 | 1.87E−12 | −0.568 | 0.038 | 0.176 | 1.50E−08 |
| SP100 | 2.29E−12 | −0.612 | 0.314 | 0.465 | 1.83E−08 |
| GBP4 | 2.66E−12 | −0.758 | 0.08 | 0.267 | 2.14E−08 |
| PARP14 | 2.78E−12 | −0.722 | 0.167 | 0.351 | 2.23E−08 |
| LPAR6 | 3.12E−12 | −0.879 | 0.239 | 0.439 | 2.50E−08 |
| EEF1A1 | 4.22E−12 | −0.440 | 0.799 | 0.791 | 3.39E−08 |
| IFI27 | 4.49E−12 | −1.543 | 0.114 | 0.255 | 3.60E−08 |
| SNX6 | 4.57E−12 | −0.714 | 0.299 | 0.446 | 3.67E−08 |
| VMP1 | 4.69E−12 | −0.588 | 0.17 | 0.296 | 3.76E−08 |
| MNDA | 4.80E−12 | −0.823 | 0.549 | 0.641 | 3.85E−08 |
| FYB | 5.78E−12 | −0.510 | 0.508 | 0.56 | 4.64E−08 |
| NMI | 5.97E−12 | −0.639 | 0.235 | 0.357 | 4.79E−08 |
| CTSL | 6.03E−12 | −0.925 | 0.087 | 0.267 | 4.84E−08 |
| NCF2 | 7.62E−12 | −0.751 | 0.17 | 0.328 | 6.12E−08 |
| CCR1 | 9.29E−12 | −0.705 | 0.068 | 0.261 | 7.45E−08 |
| RPL21 | 9.47E−12 | −0.590 | 0.148 | 0.302 | 7.60E−08 |
| NEAT1 | 1.04E−11 | −0.786 | 0.11 | 0.291 | 8.37E−08 |
| LHFPL2 | 1.29E−11 | −0.871 | 0.045 | 0.21 | 1.03E−07 |
| TMEM123 | 1.56E−11 | −0.505 | 0.489 | 0.506 | 1.25E−07 |
| CTSD | 3.01E−11 | −0.814 | 0.212 | 0.423 | 2.42E−07 |
| CTSH | 3.68E−11 | −0.586 | 0.61 | 0.706 | 2.95E−07 |
| SMCHD1 | 3.92E−11 | −0.581 | 0.174 | 0.319 | 3.15E−07 |
| SAMSN1 | 4.31E−11 | −0.735 | 0.182 | 0.397 | 3.46E−07 |
| DPYD | 4.48E−11 | −0.623 | 0.064 | 0.232 | 3.60E−07 |
| ZNF638 | 4.52E−11 | −0.444 | 0.178 | 0.247 | 3.62E−07 |
| MTRNR2L1 | 4.88E−11 | −0.659 | 0.659 | 0.719 | 3.91E−07 |
| LRP1 | 5.18E−11 | −0.658 | 0.11 | 0.282 | 4.16E−07 |
| MFSD1 | 5.56E−11 | −0.392 | 0.246 | 0.291 | 4.46E−07 |
| MS4A4A | 5.99E−11 | −0.660 | 0.091 | 0.288 | 4.81E−07 |
| ABCA1 | 6.02E−11 | −0.615 | 0.027 | 0.161 | 4.83E−07 |
| DSE | 6.05E−11 | −0.639 | 0.182 | 0.324 | 4.86E−07 |
| MTRNR2L6 | 7.01E−11 | −0.471 | 0.114 | 0.235 | 5.62E−07 |
| FKBP5 | 7.66E−11 | −0.605 | 0.114 | 0.267 | 6.14E−07 |
| TNFSF13B | 8.27E−11 | −0.594 | 0.409 | 0.511 | 6.64E−07 |
| CEP350 | 9.26E−11 | −0.555 | 0.254 | 0.345 | 7.43E−07 |
| TACC1 | 1.33E−10 | −0.474 | 0.205 | 0.35 | 1.07E−06 |
| RPL18A | 1.52E−10 | −0.505 | 0.242 | 0.336 | 1.22E−06 |
| ITGB1 | 1.60E−10 | −0.501 | 0.269 | 0.394 | 1.28E−06 |
| EIF3A | 1.70E−10 | −0.258 | 0.348 | 0.362 | 1.37E−06 |
| CLIC1 | 1.93E−10 | −0.484 | 0.201 | 0.359 | 1.55E−06 |
| DNAJA1 | 2.42E−10 | −0.539 | 0.455 | 0.537 | 1.94E−06 |
| GLUL | 2.48E−10 | −0.716 | 0.197 | 0.394 | 1.99E−06 |
| TGFBI | 2.50E−10 | −0.452 | 0.409 | 0.457 | 2.01E−06 |
| MB21D1 | 2.65E−10 | −0.593 | 0.03 | 0.178 | 2.12E−06 |
| HNRNPA2B1 | 2.69E−10 | −0.483 | 0.595 | 0.702 | 2.16E−06 |
| UTRN | 2.94E−10 | −0.684 | 0.167 | 0.328 | 2.36E−06 |
| CPVL | 3.54E−10 | −0.668 | 0.333 | 0.505 | 2.84E−06 |
| SOAT1 | 3.75E−10 | −0.501 | 0.091 | 0.238 | 3.00E−06 |
| TYROBP | 4.11E−10 | −0.419 | 0.928 | 0.902 | 3.30E−06 |
| DDX60L | 4.75E−10 | −0.486 | 0.042 | 0.181 | 3.81E−06 |
| HAVCR2 | 4.96E−10 | −0.561 | 0.083 | 0.235 | 3.98E−06 |
| DDX60 | 5.06E−10 | −0.448 | 0.027 | 0.175 | 4.06E−06 |
| ARF1 | 5.74E−10 | −0.542 | 0.367 | 0.436 | 4.61E−06 |
| CD83 | 6.48E−10 | −0.333 | 0.273 | 0.229 | 5.20E−06 |
| ACSL4 | 7.07E−10 | −0.593 | 0.167 | 0.345 | 5.68E−06 |
| EMILIN2 | 7.32E−10 | −0.553 | 0.095 | 0.236 | 5.87E−06 |
| ATF6 | 9.70E−10 | −0.501 | 0.159 | 0.267 | 7.78E−06 |
| IL1RN | 9.75E−10 | −0.699 | 0.053 | 0.215 | 7.82E−06 |
| CECR1 | 1.17E−09 | −0.506 | 0.136 | 0.293 | 9.36E−06 |
| LCP1 | 1.21E−09 | −0.439 | 0.614 | 0.626 | 9.70E−06 |
| NAMPT | 1.22E−09 | −0.609 | 0.22 | 0.351 | 9.81E−06 |
| FUCA1 | 1.23E−09 | −0.500 | 0.023 | 0.149 | 9.90E−06 |
| ADAR | 1.24E−09 | −0.524 | 0.182 | 0.348 | 9.99E−06 |
| DST | 1.41E−09 | −0.729 | 0.061 | 0.201 | 1.13E−05 |
| CTSC | 1.42E−09 | −0.505 | 0.326 | 0.422 | 1.14E−05 |
| MACF1 | 1.75E−09 | −0.670 | 0.186 | 0.324 | 1.41E−05 |
| CSNK1A1 | 1.86E−09 | −0.290 | 0.292 | 0.354 | 1.49E−05 |
| PDIA3 | 1.93E−09 | −0.490 | 0.477 | 0.609 | 1.55E−05 |
| SCARB2 | 2.00E−09 | −0.667 | 0.125 | 0.282 | 1.61E−05 |
| RAB8A | 2.41E−09 | −0.475 | 0.231 | 0.33 | 1.94E−05 |
| SERPING1 | 2.53E−09 | −0.476 | 0.038 | 0.183 | 2.03E−05 |
| PIK3AP1 | 2.65E−09 | −0.468 | 0.121 | 0.256 | 2.13E−05 |
| ARHGAP25 | 2.91E−09 | −0.410 | 0.038 | 0.181 | 2.34E−05 |
| SLFN11 | 3.14E−09 | −0.508 | 0.03 | 0.153 | 2.52E−05 |
| TRIP12 | 3.17E−09 | −0.573 | 0.114 | 0.262 | 2.54E−05 |
| CREG1 | 3.33E−09 | −0.681 | 0.129 | 0.288 | 2.67E−05 |
| TYMP | 3.52E−09 | −0.583 | 0.311 | 0.502 | 2.83E−05 |
| FOS | 4.20E−09 | −0.554 | 0.61 | 0.603 | 3.37E−05 |
| HNRNPU | 4.29E−09 | −0.557 | 0.25 | 0.419 | 3.44E−05 |
| CTSZ | 5.07E−09 | −0.555 | 0.182 | 0.371 | 4.06E−05 |
| UBE2L6 | 5.08E−09 | −0.561 | 0.152 | 0.308 | 4.08E−05 |
| LAIR1 | 5.10E−09 | −0.496 | 0.129 | 0.308 | 4.09E−05 |
| RNF13 | 5.13E−09 | −0.537 | 0.235 | 0.336 | 4.12E−05 |
| LY6E | 5.81E−09 | −0.470 | 0.235 | 0.363 | 4.66E−05 |
| CPEB4 | 6.22E−09 | −0.405 | 0.034 | 0.135 | 4.99E−05 |
| CD81 | 6.35E−09 | −0.489 | 0.091 | 0.258 | 5.09E−05 |
| STAT2 | 6.71E−09 | −0.484 | 0.072 | 0.229 | 5.38E−05 |
| IFITM1 | 6.97E−09 | −0.519 | 0.034 | 0.146 | 5.59E−05 |
| SORL1 | 8.02E−09 | −0.400 | 0.231 | 0.241 | 6.43E−05 |
| SLC11A1 | 8.54E−09 | −0.572 | 0.087 | 0.252 | 6.85E−05 |
| TMED5 | 8.68E−09 | −0.425 | 0.152 | 0.238 | 6.96E−05 |
| C6orf62 | 9.18E−09 | −0.425 | 0.258 | 0.35 | 7.37E−05 |
| FMNL2 | 9.41E−09 | −0.505 | 0.061 | 0.153 | 7.55E−05 |
| DICER1 | 9.56E−09 | −0.497 | 0.08 | 0.212 | 7.67E−05 |
| CAP1 | 9.86E−09 | −0.403 | 0.61 | 0.644 | 7.91E−05 |
| ST8SIA4 | 9.88E−09 | −0.526 | 0.083 | 0.221 | 7.92E−05 |
| H2AFY | 9.98E−09 | −0.371 | 0.39 | 0.439 | 8.01E−05 |
| LPCAT2 | 1.15E−08 | −0.562 | 0.186 | 0.308 | 9.22E−05 |
| SLC1A3 | 1.43E−08 | −0.591 | 0.049 | 0.186 | 1.15E−04 |
| DOCK4 | 1.48E−08 | −0.676 | 0.068 | 0.204 | 1.19E−04 |
| S100A9 | 1.58E−08 | −1.025 | 0.212 | 0.411 | 1.27E−04 |
| TPM3 | 1.60E−08 | −0.470 | 0.652 | 0.739 | 1.28E−04 |
| GBP5 | 1.61E−08 | −0.602 | 0.087 | 0.189 | 1.29E−04 |
| KMT2C | 1.74E−08 | −0.414 | 0.136 | 0.235 | 1.39E−04 |
| RNF149 | 1.91E−08 | −0.416 | 0.205 | 0.235 | 1.54E−04 |
| MIA3 | 1.91E−08 | −0.444 | 0.14 | 0.229 | 1.54E−04 |
| VPS13C | 1.95E−08 | −0.504 | 0.28 | 0.368 | 1.56E−04 |
| NABP1 | 1.98E−08 | −0.459 | 0.038 | 0.15 | 1.59E−04 |
| LIMS1 | 2.13E−08 | −0.381 | 0.367 | 0.399 | 1.71E−04 |
| TFEC | 2.20E−08 | −0.521 | 0.072 | 0.218 | 1.76E−04 |
| CASP4 | 2.21E−08 | −0.339 | 0.148 | 0.247 | 1.77E−04 |
| USP18 | 2.23E−08 | −0.280 | 0.004 | 0.103 | 1.79E−04 |
| PAFAH1B1 | 2.32E−08 | −0.276 | 0.205 | 0.221 | 1.86E−04 |
| ACTB | 2.37E−08 | −0.514 | 0.845 | 0.871 | 1.90E−04 |
| LGALS8 | 2.49E−08 | −0.316 | 0.201 | 0.23 | 2.00E−04 |
| AKAP13 | 2.67E−08 | −0.400 | 0.295 | 0.371 | 2.14E−04 |
| AZI2 | 2.78E−08 | −0.482 | 0.121 | 0.216 | 2.23E−04 |
| CTSA | 2.83E−08 | −0.459 | 0.08 | 0.235 | 2.27E−04 |
| WNK1 | 3.05E−08 | −0.482 | 0.098 | 0.23 | 2.45E−04 |
| F13A1 | 3.22E−08 | −0.623 | 0.072 | 0.186 | 2.59E−04 |
| P4HA1 | 3.27E−08 | −0.529 | 0.136 | 0.259 | 2.63E−04 |
| DYNLT1 | 3.56E−08 | −0.449 | 0.083 | 0.213 | 2.86E−04 |
| CD84 | 3.81E−08 | −0.539 | 0.144 | 0.298 | 3.06E−04 |
| MLEC | 3.84E−08 | −0.387 | 0.201 | 0.255 | 3.08E−04 |
| GAPDH | 3.84E−08 | −0.406 | 0.814 | 0.863 | 3.08E−04 |
| RB1 | 3.85E−08 | −0.567 | 0.212 | 0.357 | 3.09E−04 |
| EPB41L2 | 4.40E−08 | −0.590 | 0.117 | 0.267 | 3.53E−04 |
| ATP1B3 | 4.43E−08 | −0.547 | 0.144 | 0.273 | 3.56E−04 |
| FGD4 | 4.62E−08 | −0.636 | 0.087 | 0.224 | 3.71E−04 |
| LDHA | 5.04E−08 | −0.524 | 0.356 | 0.498 | 4.05E−04 |
| ANXA5 | 5.09E−08 | −0.468 | 0.496 | 0.572 | 4.09E−04 |
| CLTA | 5.13E−08 | −0.450 | 0.212 | 0.308 | 4.12E−04 |
| IFNGR1 | 6.02E−08 | −0.614 | 0.326 | 0.491 | 4.83E−04 |
| TMBIM6 | 6.39E−08 | −0.417 | 0.545 | 0.655 | 5.13E−04 |
| S100A8 | 6.48E−08 | −1.133 | 0.14 | 0.317 | 5.20E−04 |
| RIPK1 | 6.53E−08 | −0.318 | 0.057 | 0.135 | 5.24E−04 |
| EPB41L3 | 6.74E−08 | −0.531 | 0.076 | 0.233 | 5.41E−04 |
| KIDINS220 | 6.96E−08 | −0.384 | 0.144 | 0.209 | 5.58E−04 |
| TREM2 | 7.39E−08 | −0.744 | 0.216 | 0.406 | 5.93E−04 |
| GBP2 | 7.44E−08 | −0.589 | 0.148 | 0.288 | 5.97E−04 |
| DTX3L | 8.18E−08 | −0.509 | 0.098 | 0.239 | 6.56E−04 |
| SKAP2 | 8.70E−08 | −0.287 | 0.303 | 0.319 | 6.98E−04 |
| FCGR1B | 8.97E−08 | −0.359 | 0.053 | 0.183 | 7.19E−04 |
| HSD17B11 | 9.17E−08 | −0.310 | 0.341 | 0.376 | 7.36E−04 |
| CD14 | 9.35E−08 | −0.534 | 0.402 | 0.612 | 7.50E−04 |
| IGSF6 | 9.76E−08 | −0.502 | 0.326 | 0.416 | 7.83E−04 |
| STK38L | 9.85E−08 | −0.348 | 0.095 | 0.144 | 7.91E−04 |
| ATF5 | 1.02E−07 | −0.348 | 0.064 | 0.207 | 8.15E−04 |
| RCC2 | 1.03E−07 | −0.292 | 0.083 | 0.12 | 8.24E−04 |
| STX7 | 1.05E−07 | −0.435 | 0.17 | 0.265 | 8.41E−04 |
| APOC1 | 1.07E−07 | −0.908 | 0.242 | 0.422 | 8.57E−04 |
| CAPZA1 | 1.07E−07 | −0.456 | 0.333 | 0.449 | 8.59E−04 |
| SF3B1 | 1.08E−07 | −0.260 | 0.527 | 0.506 | 8.70E−04 |
| PTP4A1 | 1.09E−07 | −0.278 | 0.182 | 0.207 | 8.78E−04 |
| RPL22 | 1.21E−07 | −0.263 | 0.515 | 0.518 | 9.73E−04 |
| HIST1H1E | 1.22E−07 | −0.568 | 0.057 | 0.163 | 9.76E−04 |
| TPM4 | 1.27E−07 | −0.486 | 0.356 | 0.515 | 1.02E−03 |
| AIF1 | 1.28E−07 | −0.477 | 0.784 | 0.807 | 1.02E−03 |
| CANX | 1.30E−07 | −0.424 | 0.534 | 0.615 | 1.04E−03 |
| RAD21 | 1.38E−07 | −0.390 | 0.208 | 0.298 | 1.11E−03 |
| GNS | 1.38E−07 | −0.485 | 0.125 | 0.244 | 1.11E−03 |
| RALB | 1.52E−07 | −0.475 | 0.083 | 0.219 | 1.22E−03 |
| SETX | 1.54E−07 | −0.499 | 0.216 | 0.328 | 1.24E−03 |
| 1-Mar | 1.60E−07 | −0.527 | 0.273 | 0.431 | 1.29E−03 |
| CXorf21 | 1.65E−07 | −0.400 | 0.064 | 0.21 | 1.32E−03 |
| HSP90B1 | 1.67E−07 | −0.661 | 0.496 | 0.589 | 1.34E−03 |
| CD44 | 1.84E−07 | −0.264 | 0.326 | 0.33 | 1.48E−03 |
| HADHA | 1.85E−07 | −0.411 | 0.311 | 0.416 | 1.49E−03 |
| SP110 | 1.85E−07 | −0.492 | 0.295 | 0.434 | 1.49E−03 |
| CSF1R | 1.89E−07 | −0.503 | 0.284 | 0.434 | 1.52E−03 |
| PARP4 | 1.98E−07 | −0.458 | 0.08 | 0.202 | 1.59E−03 |
| GOLGA4 | 2.02E−07 | −0.520 | 0.17 | 0.262 | 1.62E−03 |
| DNAJC13 | 2.19E−07 | −0.301 | 0.129 | 0.178 | 1.76E−03 |
| FAM49A | 2.20E−07 | −0.442 | 0.057 | 0.127 | 1.76E−03 |
| CLEC5A | 2.26E−07 | −0.811 | 0.117 | 0.216 | 1.81E−03 |
| ZEB2 | 2.26E−07 | −0.520 | 0.246 | 0.414 | 1.81E−03 |
| OASL | 2.31E−07 | −0.294 | 0.015 | 0.11 | 1.85E−03 |
| PPT1 | 2.32E−07 | −0.459 | 0.314 | 0.462 | 1.86E−03 |
| FCN1 | 2.43E−07 | −0.875 | 0.148 | 0.299 | 1.95E−03 |
| VAMP5 | 2.68E−07 | −0.524 | 0.117 | 0.27 | 2.15E−03 |
| TIMP1 | 2.96E−07 | −0.384 | 0.28 | 0.342 | 2.37E−03 |
| ARHGAP18 | 3.01E−07 | −0.531 | 0.159 | 0.293 | 2.42E−03 |
| C3orf58 | 3.09E−07 | −0.333 | 0.117 | 0.137 | 2.48E−03 |
| KDM5A | 3.16E−07 | −0.339 | 0.152 | 0.232 | 2.53E−03 |
| KCNE3 | 3.23E−07 | −0.378 | 0.034 | 0.138 | 2.59E−03 |
| SIGLEC1 | 3.31E−07 | −0.388 | 0.03 | 0.146 | 2.65E−03 |
| CHD8 | 3.46E−07 | −0.430 | 0.091 | 0.181 | 2.77E−03 |
| PNPT1 | 3.49E−07 | −0.440 | 0.08 | 0.19 | 2.80E−03 |
| FBP1 | 3.70E−07 | −0.738 | 0.11 | 0.196 | 2.97E−03 |
| EIF4G1 | 3.75E−07 | −0.395 | 0.11 | 0.219 | 3.01E−03 |
| UBA3 | 3.81E−07 | −0.338 | 0.053 | 0.138 | 3.06E−03 |
| SRSF4 | 3.89E−07 | −0.345 | 0.095 | 0.206 | 3.12E−03 |
| LACTB | 3.92E−07 | −0.501 | 0.159 | 0.258 | 3.14E−03 |
| SF3B2 | 3.97E−07 | −0.297 | 0.182 | 0.255 | 3.18E−03 |
| IGF2R | 4.02E−07 | −0.463 | 0.027 | 0.115 | 3.23E−03 |
| PLIN2 | 4.06E−07 | −0.761 | 0.083 | 0.213 | 3.26E−03 |
| PPA1 | 4.18E−07 | −0.470 | 0.269 | 0.353 | 3.36E−03 |
| IL17RA | 4.32E−07 | −0.315 | 0.155 | 0.216 | 3.47E−03 |
| PKM | 4.58E−07 | −0.438 | 0.375 | 0.479 | 3.67E−03 |
| ACTR3 | 4.66E−07 | −0.478 | 0.439 | 0.548 | 3.74E−03 |
| COPB2 | 5.05E−07 | −0.451 | 0.095 | 0.216 | 4.05E−03 |
| PSMB9 | 5.37E−07 | −0.350 | 0.178 | 0.271 | 4.31E−03 |
| SARS | 5.39E−07 | −0.323 | 0.193 | 0.222 | 4.32E−03 |
| DAB2 | 5.55E−07 | −0.503 | 0.11 | 0.227 | 4.45E−03 |
| TMEM167A | 5.74E−07 | −0.426 | 0.121 | 0.233 | 4.60E−03 |
| C5AR1 | 5.77E−07 | −0.589 | 0.201 | 0.385 | 4.63E−03 |
| ALCAM | 5.78E−07 | −0.476 | 0.25 | 0.308 | 4.64E−03 |
| SSB | 5.90E−07 | −0.438 | 0.22 | 0.393 | 4.73E−03 |
| EPRS | 6.01E−07 | −0.300 | 0.182 | 0.245 | 4.82E−03 |
| HLA-C | 6.28E−07 | −0.370 | 0.807 | 0.83 | 5.04E−03 |
| RAB10 | 6.41E−07 | −0.428 | 0.223 | 0.324 | 5.14E−03 |
| RPN2 | 6.68E−07 | −0.322 | 0.273 | 0.334 | 5.36E−03 |
| DRAP1 | 7.14E−07 | −0.398 | 0.231 | 0.359 | 5.73E−03 |
| PHF3 | 7.28E−07 | −0.459 | 0.189 | 0.299 | 5.84E−03 |
| MT-CO1 | 7.31E−07 | −0.446 | 0.716 | 0.739 | 5.86E−03 |
| CORO1C | 7.50E−07 | −0.357 | 0.098 | 0.187 | 6.02E−03 |
| CLINT1 | 7.51E−07 | −0.316 | 0.152 | 0.207 | 6.02E−03 |
| FKBP15 | 7.52E−07 | −0.408 | 0.087 | 0.207 | 6.03E−03 |
| TXNDC12 | 7.94E−07 | −0.282 | 0.163 | 0.195 | 6.37E−03 |
| S100A11 | 8.41E−07 | −0.421 | 0.659 | 0.759 | 6.75E−03 |
| KIF5B | 8.42E−07 | −0.380 | 0.326 | 0.399 | 6.76E−03 |
| HNRNPA3 | 8.49E−07 | −0.258 | 0.242 | 0.27 | 6.81E−03 |
| YBX1 | 9.00E−07 | −0.396 | 0.576 | 0.669 | 7.22E−03 |
| PRPF40A | 9.12E−07 | −0.419 | 0.292 | 0.417 | 7.31E−03 |
| BTG1 | 9.45E−07 | −0.433 | 0.508 | 0.564 | 7.58E−03 |
| DYNC1H1 | 9.81E−07 | −0.342 | 0.125 | 0.181 | 7.87E−03 |
| PHF11 | 9.82E−07 | −0.458 | 0.197 | 0.305 | 7.88E−03 |
| C3AR1 | 1.00E−06 | −0.494 | 0.098 | 0.227 | 8.04E−03 |
| FNDC3A | 1.04E−06 | −0.402 | 0.133 | 0.199 | 8.32E−03 |
| IFIT5 | 1.06E−06 | −0.423 | 0.038 | 0.143 | 8.48E−03 |
| ADAP2 | 1.07E−06 | −0.462 | 0.125 | 0.248 | 8.60E−03 |
| MYCBP2 | 1.08E−06 | −0.388 | 0.163 | 0.253 | 8.67E−03 |
| RABGAP1L | 1.15E−06 | −0.357 | 0.152 | 0.207 | 9.21E−03 |
| CADM1 | 1.15E−06 | −0.477 | 0.034 | 0.126 | 9.21E−03 |
| NAMPTL | 1.15E−06 | −0.364 | 0.03 | 0.133 | 9.24E−03 |
| XRN1 | 1.29E−06 | −0.409 | 0.121 | 0.241 | 1.04E−02 |
| CLTC | 1.33E−06 | −0.452 | 0.186 | 0.314 | 1.06E−02 |
| YWHAE | 1.34E−06 | −0.427 | 0.254 | 0.42 | 1.07E−02 |
| C15orf38 | 1.36E−06 | −0.279 | 0.019 | 0.104 | 1.09E−02 |
| IDH1 | 1.42E−06 | −0.377 | 0.038 | 0.14 | 1.14E−02 |
| ILF3 | 1.42E−06 | −0.308 | 0.25 | 0.298 | 1.14E−02 |
| UBBP4 | 1.44E−06 | −0.335 | 0.068 | 0.187 | 1.15E−02 |
| JAK2 | 1.44E−06 | −0.306 | 0.144 | 0.195 | 1.16E−02 |
| CTTNBP2NL | 1.46E−06 | −0.515 | 0.095 | 0.195 | 1.17E−02 |
| YWHAB | 1.49E−06 | −0.290 | 0.739 | 0.712 | 1.19E−02 |
| ATP6V1B2 | 1.54E−06 | −0.363 | 0.076 | 0.201 | 1.24E−02 |
| EIF4G2 | 1.58E−06 | −0.293 | 0.432 | 0.457 | 1.27E−02 |
| RCSD1 | 1.60E−06 | −0.288 | 0.193 | 0.255 | 1.29E−02 |
| IFI35 | 1.66E−06 | −0.416 | 0.087 | 0.216 | 1.33E−02 |
| HIST1H1D | 1.79E−06 | −0.559 | 0.114 | 0.186 | 1.44E−02 |
| BAZ1A | 1.96E−06 | −0.420 | 0.155 | 0.281 | 1.58E−02 |
| CD36 | 2.00E−06 | −0.441 | 0.061 | 0.149 | 1.60E−02 |
| SNX2 | 2.05E−06 | −0.427 | 0.148 | 0.299 | 1.64E−02 |
| RNASET2 | 2.12E−06 | −0.382 | 0.511 | 0.581 | 1.70E−02 |
| COPB1 | 2.17E−06 | −0.402 | 0.144 | 0.245 | 1.74E−02 |
| RAB13 | 2.18E−06 | −0.392 | 0.057 | 0.155 | 1.75E−02 |
| 2-Sep | 2.23E−06 | −0.433 | 0.348 | 0.469 | 1.79E−02 |
| ZCCHC17 | 2.29E−06 | −0.254 | 0.095 | 0.112 | 1.83E−02 |
| SP140 | 2.31E−06 | −0.316 | 0.045 | 0.109 | 1.85E−02 |
| MAP4 | 2.33E−06 | −0.279 | 0.121 | 0.178 | 1.87E−02 |
| BACH1 | 2.37E−06 | −0.415 | 0.129 | 0.238 | 1.90E−02 |
| FAM49B | 2.59E−06 | −0.339 | 0.386 | 0.428 | 2.08E−02 |
| SH3GLB1 | 2.67E−06 | −0.470 | 0.208 | 0.337 | 2.14E−02 |
| ACIN1 | 2.68E−06 | −0.419 | 0.159 | 0.258 | 2.15E−02 |
| PDIA6 | 2.79E−06 | −0.429 | 0.242 | 0.34 | 2.24E−02 |
| SERPINB1 | 2.86E−06 | −0.479 | 0.284 | 0.38 | 2.30E−02 |
| NARS | 2.92E−06 | −0.390 | 0.197 | 0.288 | 2.34E−02 |
| FUBP1 | 2.97E−06 | −0.408 | 0.083 | 0.193 | 2.38E−02 |
| ADRBK2 | 3.12E−06 | −0.392 | 0.076 | 0.166 | 2.50E−02 |
| MYOF | 3.18E−06 | −0.389 | 0.098 | 0.21 | 2.55E−02 |
| MTHFD2 | 3.22E−06 | −0.429 | 0.129 | 0.271 | 2.59E−02 |
| USO1 | 3.34E−06 | −0.280 | 0.133 | 0.186 | 2.68E−02 |
| FERMT3 | 3.45E−06 | −0.415 | 0.087 | 0.224 | 2.77E−02 |
| ITGB2 | 3.53E−06 | −0.450 | 0.534 | 0.632 | 2.83E−02 |
| SYK | 3.56E−06 | −0.330 | 0.193 | 0.305 | 2.86E−02 |
| PICALM | 3.61E−06 | −0.478 | 0.197 | 0.311 | 2.90E−02 |
| HNMT | 3.66E−06 | −0.381 | 0.121 | 0.239 | 2.94E−02 |
| CCL2 | 3.73E−06 | −0.698 | 0.061 | 0.173 | 3.00E−02 |
| CD53 | 3.77E−06 | −0.271 | 0.458 | 0.469 | 3.03E−02 |
| RAB8B | 3.85E−06 | −0.329 | 0.11 | 0.229 | 3.09E−02 |
| ELF1 | 4.02E−06 | −0.294 | 0.322 | 0.365 | 3.22E−02 |
| ITGA4 | 4.28E−06 | −0.417 | 0.208 | 0.31 | 3.44E−02 |
| CPM | 4.39E−06 | −0.407 | 0.083 | 0.173 | 3.52E−02 |
| NCL | 4.40E−06 | −0.298 | 0.5 | 0.526 | 3.53E−02 |
| PSME1 | 4.49E−06 | −0.288 | 0.583 | 0.577 | 3.60E−02 |
| BOD1L1 | 4.51E−06 | −0.348 | 0.197 | 0.242 | 3.62E−02 |
| GSTO1 | 4.51E−06 | −0.395 | 0.242 | 0.377 | 3.62E−02 |
| TPI1 | 4.61E−06 | −0.299 | 0.439 | 0.509 | 3.70E−02 |
| PSMA4 | 5.27E−06 | −0.435 | 0.288 | 0.414 | 4.23E−02 |
| IST1 | 5.56E−06 | −0.326 | 0.076 | 0.181 | 4.46E−02 |
| GRB2 | 5.60E−06 | −0.352 | 0.621 | 0.635 | 4.49E−02 |
| PSME2 | 5.62E−06 | −0.312 | 0.322 | 0.371 | 4.51E−02 |
| CREM | 5.69E−06 | −0.536 | 0.231 | 0.294 | 4.56E−02 |
| CHMP5 | 5.82E−06 | −0.422 | 0.152 | 0.288 | 4.67E−02 |
| SNX1 | 5.85E−06 | −0.466 | 0.083 | 0.202 | 4.70E−02 |
| CAPZA2 | 5.86E−06 | −0.399 | 0.307 | 0.373 | 4.70E−02 |
| SRRM2 | 6.01E−06 | −0.364 | 0.318 | 0.373 | 4.82E−02 |
| TROVE2 | 6.22E−06 | −0.347 | 0.152 | 0.207 | 4.99E−02 |
| CD2AP | 6.23E−06 | −0.446 | 0.114 | 0.235 | 5.00E−02 |

CSF.f.mac.set.BMvLMD.markers.up.1

| | | | | | |
|---|---|---|---|---|---|
| RPL35A | 2.78E−106 | 1.127 | 0.996 | 0.943 | 2.23E−102 |
| RPL27 | 6.78E−105 | 1.182 | 1 | 0.937 | 5.44E−101 |
| RPS8 | 4.97E−102 | 1.063 | 1 | 0.949 | 3.99E−98 |
| RPS21 | 1.13E−99 | 1.194 | 1 | 0.928 | 9.05E−96 |
| RPS12 | 1.23E−99 | 1.027 | 1 | 0.966 | 9.89E−96 |
| RPL39 | 4.61E−99 | 1.390 | 1 | 0.888 | 3.70E−95 |
| RPS13 | 6.03E−98 | 1.185 | 1 | 0.887 | 4.84E−94 |
| RPL30 | 8.47E−97 | 1.086 | 1 | 0.945 | 6.79E−93 |
| RPS15A | 1.92E−95 | 1.225 | 1 | 0.847 | 1.54E−91 |
| RPS29 | 1.83E−92 | 1.137 | 1 | 0.959 | 1.47E−88 |
| RPS14 | 7.18E−88 | 0.939 | 1 | 0.951 | 5.76E−84 |
| RPS27A | 1.27E−83 | 1.071 | 0.996 | 0.86 | 1.02E−79 |
| RPL26 | 3.51E−83 | 1.127 | 1 | 0.882 | 2.81E−79 |
| TPT1 | 1.14E−79 | 0.733 | 1 | 0.98 | 9.13E−76 |
| RPL32 | 2.33E−79 | 1.050 | 0.996 | 0.899 | 1.87E−75 |
| RPS16 | 1.10E−78 | 1.056 | 1 | 0.913 | 8.81E−75 |
| RPL31 | 1.86E−78 | 1.024 | 0.996 | 0.929 | 1.49E−74 |
| RPL34 | 1.11E−77 | 0.865 | 1 | 0.91 | 8.94E−74 |
| RPLP2 | 1.29E−76 | 0.677 | 1 | 0.963 | 1.04E−72 |
| RPS18 | 1.13E−73 | 1.056 | 1 | 0.848 | 9.08E−70 |
| RPL37 | 2.00E−71 | 0.986 | 1 | 0.871 | 1.60E−67 |
| RPS24 | 5.62E−71 | 0.923 | 1 | 0.943 | 4.51E−67 |
| RPL38 | 2.90E−67 | 0.872 | 1 | 0.922 | 2.32E−63 |
| RPL11 | 1.47E−66 | 0.761 | 1 | 0.98 | 1.18E−62 |
| RPS19 | 8.11E−66 | 0.735 | 1 | 0.934 | 6.51E−62 |
| RPL19 | 7.03E−65 | 0.769 | 1 | 0.945 | 5.64E−61 |
| EEF1B2 | 1.67E−64 | 1.152 | 0.966 | 0.644 | 1.34E−60 |
| RPS6 | 8.80E−63 | 0.620 | 1 | 0.985 | 7.06E−59 |
| RPS23 | 3.70E−59 | 0.932 | 0.996 | 0.922 | 2.97E−55 |
| RPL13 | 1.52E−57 | 0.756 | 1 | 0.911 | 1.22E−53 |
| RPL5 | 6.69E−57 | 0.903 | 0.989 | 0.871 | 5.37E−53 |
| RPLP1 | 2.89E−55 | 0.667 | 1 | 0.968 | 2.32E−51 |
| RPL37A | 1.29E−52 | 0.539 | 1 | 0.939 | 1.03E−48 |
| RPS11 | 1.70E−51 | 0.653 | 1 | 0.899 | 1.36E−47 |
| RPL27A | 1.60E−50 | 0.599 | 1 | 0.939 | 1.29E−46 |
| RPS4X | 5.64E−50 | 0.740 | 0.996 | 0.824 | 4.53E−46 |
| FAU | 1.78E−48 | 0.801 | 0.996 | 0.902 | 1.43E−44 |
| RPL35 | 1.78E−48 | 0.702 | 0.992 | 0.88 | 1.43E−44 |
| RPL15 | 4.10E−47 | 0.785 | 0.992 | 0.891 | 3.29E−43 |
| HINT1 | 1.43E−44 | 0.955 | 0.871 | 0.482 | 1.15E−40 |
| RPL23A | 1.69E−44 | 0.956 | 0.928 | 0.574 | 1.35E−40 |
| RPS20 | 3.13E−44 | 0.667 | 0.992 | 0.911 | 2.51E−40 |
| RPL41 | 1.28E−41 | 0.516 | 0.996 | 0.854 | 1.03E−37 |
| RPS25 | 1.89E−41 | 0.596 | 0.958 | 0.75 | 1.51E−37 |
| RPS3 | 8.05E−41 | 0.719 | 0.985 | 0.811 | 6.46E−37 |
| UBA52 | 2.63E−40 | 0.726 | 0.981 | 0.788 | 2.11E−36 |
| RPS28 | 5.87E−37 | 0.756 | 0.958 | 0.687 | 4.71E−33 |
| RPS10 | 1.37E−36 | 0.916 | 0.871 | 0.571 | 1.10E−32 |
| RPS15 | 5.24E−36 | 0.801 | 0.966 | 0.755 | 4.20E−32 |
| PABPC1 | 1.89E−32 | 0.342 | 1 | 0.945 | 1.52E−28 |
| RPLP0 | 2.14E−30 | 0.536 | 0.973 | 0.833 | 1.72E−26 |
| RPL7A | 4.16E−28 | 0.631 | 0.917 | 0.683 | 3.34E−24 |
| ZFAS1 | 8.44E−28 | 0.814 | 0.818 | 0.491 | 6.77E−24 |
| RPS7 | 4.38E−26 | 0.558 | 0.864 | 0.518 | 3.52E−22 |
| EIF1 | 9.11E−26 | 0.552 | 0.947 | 0.79 | 7.31E−22 |
| RPS5 | 1.83E−24 | 0.586 | 0.951 | 0.747 | 1.47E−20 |
| RPL23 | 2.42E−24 | 0.346 | 0.985 | 0.873 | 1.94E−20 |
| RPL14 | 2.90E−23 | 0.528 | 0.977 | 0.821 | 2.33E−19 |
| COMMD6 | 4.49E−23 | 0.711 | 0.814 | 0.526 | 3.60E−19 |
| RPL10A | 5.44E−23 | 0.648 | 0.894 | 0.667 | 4.36E−19 |
| CD52 | 2.69E−20 | 0.612 | 0.784 | 0.489 | 2.16E−16 |
| NAP1L1 | 3.71E−20 | 0.706 | 0.799 | 0.574 | 2.97E−16 |
| CD3D | 8.86E−20 | 0.691 | 0.33 | 0.083 | 7.11E−16 |
| RPL18 | 1.34E−19 | 0.496 | 0.932 | 0.707 | 1.08E−15 |
| RPL4 | 5.01E−19 | 0.416 | 0.924 | 0.733 | 4.02E−15 |
| RPL6 | 8.07E−19 | 0.417 | 0.871 | 0.587 | 6.47E−15 |
| IL7R | 4.16E−18 | 0.770 | 0.42 | 0.138 | 3.34E−14 |
| C12orf57 | 4.86E−18 | 0.607 | 0.424 | 0.161 | 3.90E−14 |
| RPS9 | 4.01E−17 | 0.345 | 0.962 | 0.787 | 3.22E−13 |
| BTF3 | 6.58E−17 | 0.514 | 0.822 | 0.617 | 5.28E−13 |
| HLA-DPB1 | 1.61E−16 | 0.620 | 0.928 | 0.813 | 1.29E−12 |
| RPL28 | 2.44E−16 | 0.519 | 0.955 | 0.793 | 1.96E−12 |
| EEF2 | 1.63E−15 | 0.532 | 0.856 | 0.65 | 1.31E−11 |
| KLRB1 | 1.24E−14 | 0.493 | 0.258 | 0.063 | 9.94E−11 |
| RPL12 | 2.05E−14 | 0.329 | 0.917 | 0.778 | 1.64E−10 |
| TOMM7 | 6.24E−14 | 0.561 | 0.765 | 0.548 | 5.01E−10 |
| RPL9 | 1.11E−13 | 0.469 | 0.803 | 0.589 | 8.93E−10 |
| TRBC2 | 1.87E−13 | 0.591 | 0.348 | 0.137 | 1.50E−09 |
| NDUFA4 | 3.35E−13 | 0.466 | 0.867 | 0.676 | 2.68E−09 |
| PFDN5 | 4.04E−13 | 0.439 | 0.928 | 0.785 | 3.24E−09 |
| C17orf76-AS1 | 2.32E−12 | 0.498 | 0.621 | 0.385 | 1.86E−08 |
| ATP5L | 2.44E−12 | 0.447 | 0.799 | 0.578 | 1.96E−08 |
| NACA | 5.93E−12 | 0.456 | 0.86 | 0.689 | 4.76E−08 |
| ATP5E | 6.07E−12 | 0.371 | 0.951 | 0.862 | 4.87E−08 |
| SNRPD2 | 7.89E−12 | 0.406 | 0.75 | 0.498 | 6.33E−08 |
| AC013394.2 | 1.07E−11 | 0.296 | 0.235 | 0.072 | 8.57E−08 |
| TUBA1A | 1.09E−11 | 0.523 | 0.561 | 0.325 | 8.73E−08 |
| H3F3B | 1.25E−11 | 0.319 | 0.947 | 0.817 | 1.00E−07 |
| RPL8 | 1.64E−11 | 0.353 | 0.898 | 0.767 | 1.32E−07 |
| CMTM3 | 2.10E−11 | 0.291 | 0.292 | 0.12 | 1.69E−07 |
| GNB2L1 | 5.12E−11 | 0.358 | 0.92 | 0.794 | 4.10E−07 |
| HMGN3 | 6.11E−11 | 0.425 | 0.307 | 0.13 | 4.90E−07 |
| SRSF5 | 7.07E−11 | 0.436 | 0.716 | 0.479 | 5.67E−07 |
| NFKBIA | 9.66E−11 | 0.533 | 0.595 | 0.376 | 7.75E−07 |
| EIF3L | 1.10E−10 | 0.475 | 0.58 | 0.357 | 8.79E−07 |
| ZNF90 | 2.94E−10 | 0.362 | 0.428 | 0.23 | 2.35E−06 |
| S100A4 | 3.54E−10 | 0.499 | 0.811 | 0.649 | 2.84E−06 |
| PTRHD1 | 4.93E−10 | 0.406 | 0.261 | 0.097 | 3.95E−06 |
| ATP5G2 | 8.06E−10 | 0.410 | 0.727 | 0.52 | 6.46E−06 |
| MAN1A2 | 8.68E−10 | 0.521 | 0.424 | 0.215 | 6.97E−06 |
| CAMLG | 1.53E−09 | 0.361 | 0.311 | 0.15 | 1.23E−05 |
| MAP3K8 | 5.19E−09 | 0.431 | 0.273 | 0.107 | 4.16E−05 |
| KLF4 | 7.20E−09 | 0.257 | 0.273 | 0.144 | 5.78E−05 |
| COX7C | 1.24E−08 | 0.339 | 0.905 | 0.791 | 9.97E−05 |
| ARL4C | 1.44E−08 | 0.306 | 0.424 | 0.236 | 1.16E−04 |
| C8orf59 | 1.68E−08 | 0.439 | 0.519 | 0.31 | 1.35E−04 |
| MTRNR2L3 | 1.85E−08 | 0.573 | 0.492 | 0.373 | 1.48E−04 |
| OST4 | 3.83E−08 | 0.309 | 0.67 | 0.477 | 3.07E−04 |
| NDUFB1 | 4.72E−08 | 0.413 | 0.617 | 0.448 | 3.78E−04 |
| NSA2 | 6.73E−08 | 0.392 | 0.485 | 0.29 | 5.40E−04 |
| RBM3 | 1.37E−07 | 0.318 | 0.598 | 0.42 | 1.10E−03 |
| SLC25A3 | 1.69E−07 | 0.261 | 0.542 | 0.36 | 1.36E−03 |
| RPL36AL | 1.78E−07 | 0.297 | 0.674 | 0.482 | 1.43E−03 |
| IL18 | 1.81E−07 | 0.269 | 0.364 | 0.207 | 1.45E−03 |
| HLA-DMA | 2.07E−07 | 0.282 | 0.686 | 0.502 | 1.66E−03 |
| MTRNR2L8 | 2.37E−07 | 0.431 | 0.511 | 0.442 | 1.90E−03 |
| TMA7 | 2.55E−07 | 0.268 | 0.572 | 0.429 | 2.04E−03 |
| TMEM14C | 4.53E−07 | 0.349 | 0.383 | 0.25 | 3.63E−03 |
| EIF3F | 8.64E−07 | 0.371 | 0.352 | 0.195 | 6.93E−03 |
| NACA2 | 9.10E−07 | 0.363 | 0.409 | 0.245 | 7.30E−03 |
| LIMD2 | 1.22E−06 | 0.318 | 0.318 | 0.167 | 9.82E−03 |
| CKLF | 1.29E−06 | 0.372 | 0.443 | 0.291 | 1.04E−02 |
| TOPORS-AS1 | 1.32E−06 | 0.334 | 0.136 | 0.051 | 1.06E−02 |
| TSTD1 | 1.88E−06 | 0.401 | 0.284 | 0.149 | 1.51E−02 |
| ADAM15 | 3.63E−06 | 0.254 | 0.14 | 0.038 | 2.91E−02 |
| POLR1D | 4.44E−06 | 0.335 | 0.413 | 0.242 | 3.56E−02 |

TABLE 10
CSF.f.tcell.set.BMvLMD.markers

| | | | | |
|---|---|---|---|---|
| p_val | avg_logFC | pct.1 | pct.2 | p_val_adj |

CSF.f.tcell.set.BMvLMD.markers.dn.1

| | | | | | |
|---|---|---|---|---|---|
| MTRNR2L1 | 3.15E−63 | −1.456 | 0.648 | 0.805 | 2.53E−59 |
| ISG15 | 8.85E−51 | −1.269 | 0.118 | 0.497 | 7.10E−47 |
| MTRNR2L2 | 3.90E−50 | −1.194 | 0.211 | 0.501 | 3.13E−46 |
| STAT1 | 4.84E−48 | −1.090 | 0.138 | 0.514 | 3.89E−44 |
| XAF1 | 4.20E−43 | −1.038 | 0.186 | 0.545 | 3.37E−39 |
| SAMD9L | 2.98E−39 | −0.903 | 0.101 | 0.442 | 2.39E−35 |
| SAMD9 | 3.54E−39 | −1.001 | 0.152 | 0.464 | 2.84E−35 |
| CCL5 | 9.84E−39 | −1.507 | 0.487 | 0.655 | 7.89E−35 |
| IFI6 | 1.44E−37 | −0.849 | 0.09 | 0.388 | 1.16E−33 |
| GBP5 | 2.07E−36 | −0.915 | 0.166 | 0.487 | 1.66E−32 |
| RNF213 | 8.77E−36 | −0.786 | 0.262 | 0.557 | 7.04E−32 |
| MX1 | 2.45E−34 | −0.809 | 0.056 | 0.353 | 1.96E−30 |
| GBP1 | 1.04E−33 | −0.813 | 0.079 | 0.38 | 8.32E−30 |
| HLA-B | 2.89E−32 | −0.511 | 0.958 | 0.983 | 2.32E−28 |
| MTRNR2L12 | 2.49E−30 | −1.113 | 0.141 | 0.314 | 2.00E−26 |
| GZMA | 9.41E−30 | −0.927 | 0.338 | 0.575 | 7.55E−26 |
| IFIT1 | 2.03E−27 | −0.646 | 0.02 | 0.239 | 1.63E−23 |
| IFITM1 | 4.00E−26 | −0.724 | 0.115 | 0.373 | 3.21E−22 |
| IFI16 | 9.68E−26 | −0.680 | 0.324 | 0.573 | 7.77E−22 |
| EIF2AK2 | 5.77E−24 | −0.604 | 0.113 | 0.35 | 4.63E−20 |
| RSAD2 | 3.53E−23 | −0.590 | 0.02 | 0.217 | 2.83E−19 |
| IFIT3 | 3.69E−23 | −0.614 | 0.017 | 0.206 | 2.96E−19 |
| HERC5 | 6.09E−23 | −0.586 | 0.031 | 0.219 | 4.88E−19 |
| SLFN5 | 2.31E−22 | −0.650 | 0.532 | 0.67 | 1.85E−18 |
| UBB | 9.79E−22 | −0.560 | 0.586 | 0.766 | 7.85E−18 |
| HLA-E | 2.70E−21 | −0.506 | 0.856 | 0.924 | 2.17E−17 |
| IFI44L | 3.38E−21 | −0.607 | 0.039 | 0.233 | 2.71E−17 |
| B2M | 5.47E−21 | −0.313 | 1 | 1 | 4.39E−17 |
| SRGN | 1.97E−20 | −0.555 | 0.586 | 0.705 | 1.58E−16 |
| HLA-A | 1.94E−19 | −0.480 | 0.823 | 0.901 | 1.56E−15 |
| PPP2R5C | 4.21E−19 | −0.530 | 0.223 | 0.415 | 3.38E−15 |
| GIMAP4 | 6.31E−19 | −0.546 | 0.386 | 0.55 | 5.06E−15 |
| CST7 | 9.75E−19 | −0.601 | 0.208 | 0.435 | 7.83E−15 |
| PTPRC | 3.23E−18 | −0.481 | 0.834 | 0.888 | 2.59E−14 |
| GZMK | 5.46E−18 | −0.718 | 0.245 | 0.417 | 4.38E−14 |
| RPS27 | 5.50E−18 | −0.272 | 0.882 | 0.748 | 4.41E−14 |
| ETS1 | 6.25E−18 | −0.497 | 0.614 | 0.786 | 5.02E−14 |
| MT2A | 6.51E−18 | −0.565 | 0.186 | 0.392 | 5.22E−14 |
| APOL6 | 6.84E−18 | −0.528 | 0.175 | 0.401 | 5.48E−14 |
| LCP1 | 9.80E−18 | −0.510 | 0.482 | 0.643 | 7.86E−14 |
| RPL18A | 3.26E−17 | −0.429 | 0.206 | 0.353 | 2.62E−13 |
| DTX3L | 3.80E−17 | −0.393 | 0.054 | 0.231 | 3.05E−13 |
| NT5C3A | 4.43E−17 | −0.502 | 0.056 | 0.231 | 3.55E−13 |
| SP100 | 6.28E−17 | −0.551 | 0.27 | 0.495 | 5.04E−13 |
| DRAP1 | 6.42E−17 | −0.414 | 0.132 | 0.308 | 5.15E−13 |
| ISG20 | 7.39E−17 | −0.517 | 0.211 | 0.414 | 5.93E−13 |
| SH2D1A | 2.02E−16 | −0.536 | 0.175 | 0.387 | 1.62E−12 |
| CNOT6L | 3.10E−16 | −0.445 | 0.107 | 0.277 | 2.49E−12 |
| OAS1 | 3.45E−16 | −0.371 | 0.042 | 0.219 | 2.77E−12 |
| OAS2 | 3.50E−16 | −0.421 | 0.062 | 0.249 | 2.81E−12 |
| UBE2L6 | 4.90E−16 | −0.365 | 0.093 | 0.284 | 3.93E−12 |
| PIK3R1 | 6.24E−16 | −0.545 | 0.279 | 0.403 | 5.01E−12 |
| DDX17 | 6.29E−16 | −0.482 | 0.259 | 0.465 | 5.05E−12 |
| APOBEC3G | 6.77E−16 | −0.416 | 0.09 | 0.289 | 5.43E−12 |
| PYHIN1 | 5.23E−15 | −0.457 | 0.104 | 0.301 | 4.19E−11 |
| STK17A | 6.91E−15 | −0.466 | 0.158 | 0.36 | 5.54E−11 |
| GBP4 | 8.79E−15 | −0.487 | 0.099 | 0.275 | 7.05E−11 |
| ACTR2 | 1.10E−14 | −0.416 | 0.335 | 0.508 | 8.79E−11 |
| EMB | 1.76E−14 | −0.459 | 0.383 | 0.537 | 1.42E−10 |
| DDX60 | 1.91E−14 | −0.350 | 0.039 | 0.195 | 1.53E−10 |
| GBP2 | 4.08E−14 | −0.423 | 0.155 | 0.357 | 3.27E−10 |
| TRIM22 | 4.52E−14 | −0.429 | 0.101 | 0.283 | 3.63E−10 |
| EZR | 7.52E−14 | −0.430 | 0.259 | 0.437 | 6.03E−10 |
| CYBA | 8.13E−14 | −0.343 | 0.11 | 0.281 | 6.52E−10 |
| JUNB | 9.59E−14 | −0.264 | 0.203 | 0.252 | 7.69E−10 |
| IFI44 | 1.36E−13 | −0.362 | 0.051 | 0.197 | 1.09E−09 |
| TIGIT | 1.71E−13 | −0.496 | 0.099 | 0.278 | 1.37E−09 |
| PLEK | 1.76E−13 | −0.361 | 0.048 | 0.175 | 1.41E−09 |
| PARP9 | 2.25E−13 | −0.417 | 0.096 | 0.277 | 1.81E−09 |
| EPSTI1 | 3.41E−13 | −0.466 | 0.152 | 0.343 | 2.73E−09 |
| IFITM2 | 3.51E−13 | −0.426 | 0.29 | 0.451 | 2.82E−09 |
| WIPF1 | 4.64E−13 | −0.434 | 0.315 | 0.493 | 3.72E−09 |
| HIST1H1E | 6.06E−13 | −0.388 | 0.062 | 0.174 | 4.86E−09 |
| HNRNPC | 6.21E−13 | −0.352 | 0.248 | 0.415 | 4.98E−09 |
| EEF1A1 | 7.75E−13 | −0.375 | 0.701 | 0.734 | 6.21E−09 |
| CCL4 | 1.01E−12 | −0.502 | 0.042 | 0.17 | 8.08E−09 |
| RPL3 | 1.03E−12 | −0.344 | 0.718 | 0.728 | 8.23E−09 |
| SMCHD1 | 1.13E−12 | −0.398 | 0.251 | 0.382 | 9.06E−09 |
| PTPRCAP | 2.03E−12 | −0.395 | 0.58 | 0.692 | 1.63E−08 |
| OAZ1 | 2.12E−12 | −0.407 | 0.344 | 0.512 | 1.70E−08 |
| KLRD1 | 2.46E−12 | −0.546 | 0.076 | 0.245 | 1.97E−08 |
| MTRNR2L6 | 3.42E−12 | −0.323 | 0.087 | 0.239 | 2.74E−08 |
| PSME1 | 3.42E−12 | −0.387 | 0.549 | 0.634 | 2.74E−08 |
| GZMH | 3.42E−12 | −0.415 | 0.045 | 0.189 | 2.74E−08 |
| LAP3 | 3.56E−12 | −0.332 | 0.051 | 0.201 | 2.86E−08 |
| RARRES3 | 4.31E−12 | −0.377 | 0.279 | 0.406 | 3.46E−08 |
| PSMB9 | 4.93E−12 | −0.376 | 0.163 | 0.333 | 3.96E−08 |
| NMI | 5.86E−12 | −0.382 | 0.113 | 0.263 | 4.70E−08 |
| HIST1H1D | 6.12E−12 | −0.486 | 0.11 | 0.29 | 4.91E−08 |
| MSN | 7.49E−12 | −0.373 | 0.231 | 0.394 | 6.01E−08 |
| TANK | 8.53E−12 | −0.381 | 0.107 | 0.248 | 6.84E−08 |
| CD69 | 1.21E−11 | −0.555 | 0.287 | 0.406 | 9.72E−08 |
| IFIT2 | 1.33E−11 | −0.339 | 0.031 | 0.143 | 1.07E−07 |
| ERAP2 | 1.55E−11 | −0.332 | 0.054 | 0.191 | 1.24E−07 |
| LYST | 1.59E−11 | −0.395 | 0.079 | 0.239 | 1.28E−07 |
| ACTB | 1.65E−11 | −0.433 | 0.623 | 0.718 | 1.32E−07 |
| RAD21 | 2.46E−11 | −0.369 | 0.197 | 0.354 | 1.97E−07 |
| RPL21 | 2.80E−11 | −0.333 | 0.135 | 0.237 | 2.25E−07 |
| MTRNR2L11 | 3.23E−11 | −0.319 | 0.138 | 0.237 | 2.59E−07 |
| SYNE2 | 3.30E−11 | −0.503 | 0.454 | 0.594 | 2.65E−07 |
| CD8A | 3.45E−11 | −0.495 | 0.076 | 0.214 | 2.77E−07 |
| STK17B | 3.57E−11 | −0.347 | 0.304 | 0.446 | 2.87E−07 |
| CAP1 | 3.66E−11 | −0.387 | 0.346 | 0.527 | 2.94E−07 |
| ARHGDIB | 4.07E−11 | −0.378 | 0.623 | 0.732 | 3.26E−07 |
| CTSS | 5.31E−11 | −0.393 | 0.239 | 0.398 | 4.26E−07 |
| IRF1 | 5.73E−11 | −0.368 | 0.118 | 0.284 | 4.60E−07 |
| SPATS2L | 7.03E−11 | −0.251 | 0.008 | 0.103 | 5.64E−07 |
| IFIH1 | 7.69E−11 | −0.377 | 0.073 | 0.216 | 6.17E−07 |
| ARL4C | 8.00E−11 | −0.281 | 0.307 | 0.364 | 6.42E−07 |
| CD96 | 8.03E−11 | −0.328 | 0.304 | 0.424 | 6.44E−07 |
| UBBP4 | 1.13E−10 | −0.282 | 0.07 | 0.223 | 9.09E−07 |
| CAPN2 | 1.22E−10 | −0.296 | 0.166 | 0.284 | 9.80E−07 |
| ANXA5 | 1.41E−10 | −0.364 | 0.197 | 0.317 | 1.13E−06 |
| CCR5 | 1.44E−10 | −0.351 | 0.065 | 0.206 | 1.16E−06 |
| NCL | 1.82E−10 | −0.307 | 0.408 | 0.475 | 1.46E−06 |
| BST2 | 2.09E−10 | −0.317 | 0.13 | 0.255 | 1.68E−06 |
| EVL | 2.62E−10 | −0.324 | 0.324 | 0.424 | 2.10E−06 |
| TAP1 | 2.75E−10 | −0.327 | 0.135 | 0.287 | 2.20E−06 |
| LINC00152 | 4.62E−10 | −0.276 | 0.065 | 0.206 | 3.71E−06 |
| LITAF | 4.69E−10 | −0.280 | 0.192 | 0.305 | 3.77E−06 |
| YWHAE | 5.61E−10 | −0.279 | 0.093 | 0.228 | 4.50E−06 |
| SP110 | 8.59E−10 | −0.352 | 0.228 | 0.353 | 6.90E−06 |
| SAMD3 | 9.25E−10 | −0.386 | 0.149 | 0.293 | 7.42E−06 |
| CXCR4 | 1.11E−09 | −0.301 | 0.465 | 0.496 | 8.87E−06 |
| CREM | 1.28E−09 | −0.403 | 0.051 | 0.163 | 1.03E−05 |
| ACTR3 | 1.43E−09 | −0.353 | 0.268 | 0.439 | 1.15E−05 |
| HLA-C | 1.44E−09 | −0.312 | 0.831 | 0.874 | 1.16E−05 |
| BRD2 | 1.62E−09 | −0.299 | 0.149 | 0.295 | 1.30E−05 |
| CLK1 | 1.90E−09 | −0.312 | 0.127 | 0.26 | 1.53E−05 |
| RGS1 | 2.40E−09 | −0.539 | 0.237 | 0.356 | 1.92E−05 |
| TMEM123 | 2.62E−09 | −0.301 | 0.372 | 0.436 | 2.10E−05 |
| GIMAP2 | 3.45E−09 | −0.286 | 0.121 | 0.212 | 2.77E−05 |
| DDX58 | 4.10E−09 | −0.278 | 0.037 | 0.151 | 3.29E−05 |
| TLN1 | 4.41E−09 | −0.314 | 0.231 | 0.338 | 3.54E−05 |
| CCND2 | 4.44E−09 | −0.320 | 0.161 | 0.302 | 3.56E−05 |
| HIST1H4C | 4.84E−09 | −0.309 | 0.47 | 0.39 | 3.88E−05 |
| KIAA1551 | 6.77E−09 | −0.350 | 0.389 | 0.478 | 5.43E−05 |
| PTPN22 | 8.00E−09 | −0.276 | 0.065 | 0.169 | 6.42E−05 |
| CD74 | 8.25E−09 | −0.375 | 0.507 | 0.575 | 6.62E−05 |
| IQGAP2 | 1.12E−08 | −0.307 | 0.11 | 0.211 | 8.96E−05 |
| CLEC2D | 1.19E−08 | −0.321 | 0.206 | 0.335 | 9.53E−05 |
| PAIP2 | 1.21E−08 | −0.313 | 0.166 | 0.308 | 9.70E−05 |
| TPM3 | 1.39E−08 | −0.346 | 0.465 | 0.602 | 1.12E−04 |
| MX2 | 1.80E−08 | −0.342 | 0.096 | 0.223 | 1.45E−04 |
| RBL2 | 1.94E−08 | −0.309 | 0.268 | 0.373 | 1.56E−04 |
| HNRNPU | 2.01E−08 | −0.279 | 0.197 | 0.325 | 1.61E−04 |
| HNRNPA2B1 | 2.08E−08 | −0.341 | 0.462 | 0.576 | 1.67E−04 |
| TNFSF10 | 2.10E−08 | −0.300 | 0.127 | 0.206 | 1.69E−04 |
| SETX | 2.10E−08 | −0.321 | 0.144 | 0.259 | 1.69E−04 |
| SCAF11 | 2.31E−08 | −0.257 | 0.33 | 0.379 | 1.85E−04 |
| MT-ND3 | 2.70E−08 | −0.340 | 0.594 | 0.683 | 2.17E−04 |
| PSME2 | 2.88E−08 | −0.306 | 0.189 | 0.34 | 2.31E−04 |
| RPL17 | 2.96E−08 | −0.300 | 0.144 | 0.262 | 2.38E−04 |
| F2R | 3.04E−08 | −0.262 | 0.048 | 0.156 | 2.44E−04 |
| PTPN4 | 3.55E−08 | −0.275 | 0.076 | 0.175 | 2.85E−04 |
| TMPO | 3.94E−08 | −0.281 | 0.121 | 0.239 | 3.16E−04 |
| ILF3 | 4.12E−08 | −0.313 | 0.121 | 0.246 | 3.31E−04 |
| CMC1 | 5.45E−08 | −0.567 | 0.183 | 0.253 | 4.37E−04 |
| C11orf58 | 5.46E−08 | −0.313 | 0.251 | 0.398 | 4.38E−04 |
| G3BP2 | 5.94E−08 | −0.299 | 0.166 | 0.259 | 4.76E−04 |
| RP11-94L15.2 | 7.46E−08 | −0.285 | 0.11 | 0.224 | 5.99E−04 |
| IFIT5 | 8.20E−08 | −0.271 | 0.051 | 0.147 | 6.58E−04 |
| ITGB2 | 1.05E−07 | −0.287 | 0.223 | 0.326 | 8.43E−04 |
| HIST1H1C | 1.16E−07 | −0.316 | 0.065 | 0.169 | 9.28E−04 |
| SYNE1 | 1.25E−07 | −0.287 | 0.138 | 0.243 | 1.01E−03 |
| NUB1 | 1.35E−07 | −0.281 | 0.155 | 0.251 | 1.08E−03 |
| ITGAL | 1.46E−07 | −0.258 | 0.121 | 0.235 | 1.17E−03 |
| LNPEP | 1.49E−07 | −0.289 | 0.099 | 0.183 | 1.19E−03 |
| GNG2 | 1.87E−07 | −0.278 | 0.115 | 0.241 | 1.50E−03 |
| DNAJA1 | 1.88E−07 | −0.262 | 0.248 | 0.33 | 1.51E−03 |
| C12orf75 | 2.12E−07 | −0.266 | 0.096 | 0.207 | 1.70E−03 |
| SAT1 | 2.40E−07 | −0.338 | 0.197 | 0.295 | 1.92E−03 |
| ADAR | 3.12E−07 | −0.254 | 0.085 | 0.21 | 2.51E−03 |
| CD44 | 3.47E−07 | −0.289 | 0.31 | 0.4 | 2.79E−03 |
| PARP14 | 3.69E−07 | −0.272 | 0.113 | 0.205 | 2.96E−03 |
| ITGB1 | 3.89E−07 | −0.332 | 0.375 | 0.455 | 3.12E−03 |
| JAK1 | 4.84E−07 | −0.295 | 0.293 | 0.426 | 3.88E−03 |
| OPTN | 6.19E−07 | −0.280 | 0.287 | 0.381 | 4.97E−03 |
| SH3GLB1 | 6.85E−07 | −0.257 | 0.152 | 0.247 | 5.50E−03 |
| ITGA4 | 7.20E−07 | −0.302 | 0.254 | 0.389 | 5.78E−03 |
| STK4 | 7.94E−07 | −0.272 | 0.27 | 0.353 | 6.37E−03 |
| CALM3 | 8.20E−07 | −0.259 | 0.118 | 0.237 | 6.58E−03 |
| TNFAIP3 | 8.57E−07 | −0.271 | 0.138 | 0.219 | 6.87E−03 |
| JUN | 9.06E−07 | −0.250 | 0.203 | 0.237 | 7.27E−03 |
| SLA | 1.14E−06 | −0.275 | 0.124 | 0.217 | 9.17E−03 |
| XRN1 | 1.22E−06 | −0.283 | 0.124 | 0.235 | 9.79E−03 |
| USP8 | 1.43E−06 | −0.260 | 0.068 | 0.178 | 1.15E−02 |
| MTRNR2L7 | 1.44E−06 | −0.261 | 0.056 | 0.129 | 1.16E−02 |
| DUSP2 | 1.96E−06 | −0.276 | 0.082 | 0.167 | 1.58E−02 |
| ARAP2 | 2.35E−06 | −0.269 | 0.09 | 0.185 | 1.89E−02 |
| ACTG1 | 3.02E−06 | −0.303 | 0.603 | 0.676 | 2.42E−02 |
| C6orf62 | 3.18E−06 | −0.260 | 0.141 | 0.255 | 2.56E−02 |
| ARHGAP30 | 3.49E−06 | −0.252 | 0.166 | 0.282 | 2.80E−02 |
| SF3B1 | 3.55E−06 | −0.250 | 0.456 | 0.521 | 2.85E−02 |
| LYAR | 4.59E−06 | −0.289 | 0.22 | 0.318 | 3.68E−02 |
| MT-RNR1 | 4.60E−06 | −0.532 | 0.924 | 0.942 | 3.69E−02 |
| GIMAP7 | 5.25E−06 | −0.302 | 0.6 | 0.651 | 4.21E−02 |
| PDCD4 | 5.26E−06 | −0.267 | 0.214 | 0.312 | 4.22E−02 |

CSF.f.tcell.set.BMvLMD.markers.up.1

| | | | | | |
|---|---|---|---|---|---|
| RPL39 | 2.98E−147 | 1.276 | 0.997 | 0.956 | 2.39E−143 |
| RPS29 | 6.79E−122 | 0.861 | 1 | 0.999 | 5.45E−118 |
| RPL27 | 1.46E−102 | 0.827 | 1 | 0.981 | 1.17E−98 |
| RPS27A | 2.09E−102 | 0.881 | 0.997 | 0.96 | 1.67E−98 |
| RPS15A | 1.18E−97 | 0.794 | 1 | 0.978 | 9.46E−94 |
| RPS21 | 3.03E−91 | 0.797 | 1 | 0.979 | 2.43E−87 |
| RPL30 | 1.67E−88 | 0.667 | 1 | 0.998 | 1.34E−84 |
| RPS13 | 2.64E−88 | 0.866 | 0.997 | 0.942 | 2.12E−84 |
| RPS8 | 2.67E−84 | 0.714 | 1 | 0.979 | 2.14E−80 |
| RPS18 | 3.45E−80 | 0.738 | 1 | 0.866 | 2.77E−76 |
| RPL31 | 4.01E−76 | 0.636 | 1 | 0.987 | 3.21E−72 |
| RPL35A | 2.51E−74 | 0.647 | 1 | 0.992 | 2.01E−70 |
| RPL32 | 2.55E−74 | 0.755 | 1 | 0.96 | 2.05E−70 |
| RPL26 | 7.63E−70 | 0.682 | 0.994 | 0.962 | 6.13E−66 |
| RPS12 | 6.75E−69 | 0.610 | 1 | 0.996 | 5.42E−65 |
| EEF1B2 | 1.15E−64 | 0.862 | 0.904 | 0.596 | 9.24E−61 |
| RPL41 | 6.82E−59 | 0.634 | 0.989 | 0.916 | 5.47E−55 |
| RPS10 | 7.68E−58 | 0.803 | 0.865 | 0.526 | 6.16E−54 |
| RPS14 | 2.20E−56 | 0.488 | 1 | 0.974 | 1.76E−52 |
| RPL11 | 4.55E−52 | 0.523 | 1 | 0.99 | 3.65E−48 |
| RPS28 | 1.23E−51 | 0.744 | 0.932 | 0.714 | 9.88E−48 |
| RPS16 | 7.15E−51 | 0.612 | 1 | 0.975 | 5.73E−47 |
| TPT1 | 8.04E−51 | 0.416 | 1 | 0.992 | 6.45E−47 |
| RPLP2 | 1.03E−50 | 0.492 | 1 | 0.982 | 8.23E−47 |
| RPS23 | 1.91E−50 | 0.586 | 1 | 0.961 | 1.53E−46 |
| RPL5 | 3.35E−50 | 0.620 | 0.986 | 0.902 | 2.69E−46 |
| RPS25 | 1.21E−48 | 0.584 | 0.992 | 0.826 | 9.73E−45 |
| RPL38 | 3.33E−40 | 0.562 | 1 | 0.977 | 2.67E−36 |
| ZFAS1 | 1.94E−39 | 0.730 | 0.769 | 0.486 | 1.56E−35 |
| RPL23A | 5.68E−39 | 0.549 | 0.949 | 0.736 | 4.55E−35 |
| RPL19 | 2.80E−34 | 0.413 | 0.997 | 0.987 | 2.25E−30 |
| RPL37 | 3.53E−34 | 0.412 | 1 | 0.977 | 2.83E−30 |
| RPL13 | 5.48E−34 | 0.447 | 0.997 | 0.973 | 4.39E−30 |
| RPL34 | 6.59E−33 | 0.419 | 1 | 0.986 | 5.29E−29 |
| RPL15 | 7.08E−33 | 0.461 | 0.992 | 0.902 | 5.68E−29 |
| RPS4X | 9.44E−32 | 0.437 | 0.994 | 0.933 | 7.58E−28 |
| RPL27A | 3.48E−31 | 0.383 | 1 | 0.99 | 2.79E−27 |
| CD52 | 5.74E−31 | 0.442 | 0.989 | 0.896 | 4.60E−27 |
| FAU | 3.07E−29 | 0.381 | 0.989 | 0.957 | 2.46E−25 |
| RPL35 | 6.32E−29 | 0.469 | 0.989 | 0.925 | 5.07E−25 |
| CMC2 | 1.22E−28 | 0.959 | 0.231 | 0.142 | 9.81E−25 |
| RPS19 | 6.55E−25 | 0.293 | 1 | 0.979 | 5.26E−21 |
| RPS6 | 9.18E−25 | 0.277 | 1 | 0.996 | 7.36E−21 |
| RPLP0 | 3.47E−24 | 0.428 | 0.949 | 0.794 | 2.79E−20 |
| RPS7 | 1.41E−23 | 0.363 | 0.814 | 0.52 | 1.13E−19 |
| PFDN5 | 4.53E−23 | 0.465 | 0.893 | 0.725 | 3.64E−19 |
| RPS15 | 8.46E−23 | 0.437 | 0.966 | 0.891 | 6.79E−19 |
| RPL28 | 3.02E−22 | 0.420 | 0.963 | 0.89 | 2.42E−18 |
| UBA52 | 2.66E−20 | 0.404 | 0.955 | 0.842 | 2.13E−16 |
| RPS20 | 5.31E−20 | 0.316 | 0.994 | 0.981 | 4.26E−16 |
| RPL10A | 1.49E−19 | 0.385 | 0.907 | 0.734 | 1.19E−15 |
| RPS3 | 3.83E−19 | 0.335 | 0.997 | 0.939 | 3.07E−15 |
| EIF1 | 7.33E−19 | 0.385 | 0.935 | 0.793 | 5.88E−15 |
| RPLP1 | 5.15E−17 | 0.261 | 1 | 0.995 | 4.13E−13 |
| C12orf57 | 1.69E−16 | 0.466 | 0.572 | 0.363 | 1.36E−12 |
| CST3 | 1.94E−16 | 0.286 | 0.175 | 0.031 | 1.56E−12 |
| GPR183 | 2.46E−15 | 0.329 | 0.386 | 0.172 | 1.98E−11 |
| TOMM7 | 6.73E−14 | 0.359 | 0.831 | 0.669 | 5.40E−10 |
| RPL9 | 1.31E−13 | 0.392 | 0.704 | 0.514 | 1.05E−09 |
| RPL6 | 4.56E−13 | 0.297 | 0.797 | 0.599 | 3.66E−09 |
| COMMD6 | 7.96E−12 | 0.366 | 0.704 | 0.527 | 6.39E−08 |
| ZNF90 | 1.51E−11 | 0.296 | 0.417 | 0.226 | 1.21E−07 |
| RPS5 | 2.05E−11 | 0.326 | 0.89 | 0.775 | 1.65E−07 |
| HINT1 | 2.27E−11 | 0.351 | 0.755 | 0.635 | 1.82E−07 |
| RPL7A | 2.38E−11 | 0.326 | 0.868 | 0.765 | 1.91E−07 |
| S100A4 | 7.56E−10 | 0.307 | 0.789 | 0.621 | 6.06E−06 |
| FXYD5 | 1.77E−09 | 0.289 | 0.406 | 0.229 | 1.42E−05 |
| NDUFA4 | 4.56E−09 | 0.328 | 0.707 | 0.552 | 3.66E−05 |

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A method for detecting, monitoring or prognosing a cancer in a subject in need thereof comprising:
a) obtaining a biological sample comprising tumor cells from an extracellular fluid or compartment of the subject;
b) detecting in the biological sample the expression or activity of a gene signature associated with sensitivity to a therapy or response to a therapy; and
c) determining that the solid tumor present, is responding to a therapy or is capable of responding to a therapy.

2. The method of claim 1, wherein the gene signature is detected in single cells from the biological sample; and/or
wherein the biological sample comprising tumor cells is obtained from cerebral spinal fluid (CSF); or
wherein the biological sample comprising tumor cells is obtained from draining lymph nodes.

3. (canceled)

4. (canceled)

5. The method of claim 1, wherein the gene signature is associated with a response to immunotherapy, preferably, wherein the immunotherapy comprises one or more check point inhibitors, more preferably, wherein the one or more check point inhibitors comprise anti-CTLA4, anti-PD-L1 and/or anti-PD1 therapy.

6. (canceled)

7. (canceled)

8. The method of claim 1, wherein the signature exhibits upregulation of (i) genes involved in interferon regulation; (ii) genes involved in interferon response; (iii) genes involved in antigen presentation; and/or (iv) genes involved in cytotoxic T cell activation compared to the transcriptome profile of the reference sample; or
wherein the gene signature comprises one or more genes or polypeptides selected from the group consisting of:
a) AHNAK, ANKRD30B, BTG1, BZW1, C8orf4, COX6C, DSP, DUSP1, EEF1A1, EEF1D, EGR1, EIF2S2, FOS, FOSB, FTH1, GOLGB1, HES1, IRX2, JUN, JUNB, MALAT1, MGEA5, MLPH, MORF4L1, NDRG1, NEAT1, NR4A2, NUPR1, RB1CC1, RNA28S5, RPL10, RPL10A, RPL12, RPL13A, RPL18A, RPL22, RPL23A, RPL26, RPL3, RPL36, RPL37, RPL4, RPL41, RPL6, RPS15, RPS15A, RPS18, RPS2, RPS20, RPS25, RPS27, RPS27A, RPS28, RPS29, RPS7, RPSA, S100A8, S100A9, SCGB1D2, SLK, SNHG9, SRRM2, TMEM14C, TRIB1, TRPS1, UBC, UQCRB and ZFAS1; or
b) AARD, ACP1, ACTR2, ACTR3, ACTR6, AIMP1, ANP32B, AP2M1, APIP, APOD, APOL6, ARF1, ATAD2, ATP5G3, B2M, C1orf43, CALM1, CCT3, CCT4, CCT5, CCT6A, CD44, CD46, CD74, CHCHD2, COA6, COPS2, COX7A2, COX7C, COX8A, CP, EBNA1BP2, EIF2AK2, EIF5, EPRS, EZH2, FKBP3, GBP1, GTF3A, H2AFZ, HDGF, HIST1H1D, HIST1H4C, HLA-B, HLA-C, HNRNPA2B1, HSBP1, HSP90B1, IFI16, IFI27 IFI44L, IFI6, IFIH1, IFIT3, IFNAR1, IL6ST, ISG15, LAMP2, LMAN1, MAPKAP1, MDH1, MED10, MGP, MRPL13, MX1, NDUFA1, NDUFA4, NDUFB2, NUCB2, OAZ1, PAICS, PALLD, PAPOLA, PARP1, PARP9, PDCD5, PDHX, PDIA6, PEG10, PGRMC1, PKIB, PPM1G, PPP1CB, PSMA3, PSMB8, PSME1, PSME2, PTGES3, RAN, RARRES1, RHOA, RSRC1, S100A6, SEC61B, SEC62, SIVA1, SLC38A1, SMARCA5, SNX6, SRP72, SSR3, SSR4, STMN1, TAP1, TCP1, TM4SF1, TMCO1, TMEM59, TMEM97, TMPO, TRAM1, TSPO, TXNL1, UBE2J1, UBL5, UCHL5, UQCRH, USP1, VBP1 and XAF1; or
c) ACTB, AHNAK, BTG1, BZW1, C6orf62, C8orf59, COX6C, DANCR, DUSP1, EEF1B2, EEF1D, EGR1, EIF2S2, FOS, FTH1, FTL, HES1, HNRNPA1, JUNB, METTL12, MIF, MORF4L1, MT-ATP6, MT-ATP8, MT-CO1, MT-CO2, MT-CO3, MT-CYB, MT-ND1, MT-ND2, MT-ND4, MT-ND4L, MT-ND5, MT-ND6, MT-RNR1, MT-TL1, MT-TV, MTRNR2L1, MTRNR2L11, MTRNR2L12, MTRNR2L2, MTRNR2L3, MTRNR2L8, NDUFB1, NEAT1, NR4A2, PCBP2, PET100, PFDN5, PPDPF, PRDX2, RB1CC1, RNA18S5, RNA28S5, RNY1, ROMO1, RPL10, RPL10A, RPL12, RPL13, RPL13A, RPL15, RPL18, RPL18A, RPL22, RPL23A, RPL26, RPL27A, RPL28, RPL29, RPL31, RPL32, RPL34, RPL35, RPL36, RPL37, RPL39, RPL4, RPL41, RPL6, RPL7A, RPL8, RPL9, RPN2, RPS14, RPS15, RPS15A, RPS18, RPS19, RPS2, RPS20, RPS25, RPS27, RPS27A, RPS28, RPS29, RPS3, RPS3A, RPS4X, RPS7, RPS9, RPSA, S100A8, SLK, SNHG9, SYNGR2, TMA7, TMSB10, TMSB4X, UBC and UQCRB; or
d) ACBD3, ACP1, ACTR2, ACTR3, ACTR6, ADSS, AMD1, AP2M1, APEX1, ARF1, ARPC5, ASH1L, ATAD2, ATP5G3, B2M, BROX, C1orf21, C1orf43, CALM1, CANX, CAPN2, CAPRIN1, CCDC59, CCNC, CCT2, CCT3, CCT4, CCT6A, CD109, CD46, CDC16, CDCA7L, CLTC, CMPK1, CNBP, COA6, COPS2, CP, DENND1B, DHX9, DNAJC3, DST, DSTN, ECT2, EID1, EIF2AK2, EIF2S1, EIF3D, EIF4A2, EIF5, EMC4, ENO1, EPRS, EZH2, FKBP3, GADD45GIP1, GMPS, GTF3A, GTPBP4, HADHA, HDGF, HDLBP, HIST1H1D, HIST1H4C, HNRNPA2B1, HSP90B1, HSPA5, HSPH1, IFNAR1, IQGAP1, LAMP2, LMAN1, LPP, LRPPRC, MAN1A2, MAPKAP1, MBNL1, MBNL2, MED10, MORF4L2, MRPL13, MRPL42, MRPS35, MX1, NASP, NOL11, NSMCE2, NUCB2, NUCKS1, NUDCD1, PAICS, PAPOLA, PARP1, PDCD10, PDHX, PDIA6, PGRMC1, PIK3R1, PLRG1, PMAIP1, PMP22, POLR2B, PPM1G, PRPF40A, PSMA3, PSME4, PTBP3, PTTG1IP, RAB10, RAD21, RAN, RARRES1, RHOA, RNF168, RSRC1, S100A6, SAP30BP, SCRN1, SEC63, SENP6, SF3B1, SLC38A1, SMARCA5, SMC3, SMEK2, SNX6, SPEN, SRP72, SRSF3, SSR3, SSRP1, ST3GAL1, SUCO, TCP1, TKT, TM4SF1, TMPO, TOMM20, TOMM70A, TP53BP2, TPM1, TRAM1, TXNL1, UBE2J1, UBE2K, UCHL5, UGP2, USP1, USP16, VBP1, VIM, XIST, ZC3H14 and ZDHHC20; or
e) COX6C, EEF1D, RNA28S5, RPL10, RPL12, RPL13A, RPL18A, RPL23A, RPL26, RPL36, RPL41, RPL6, RPS15, RPS15A, RPS18, RPS27, RPS27A, RPS28, RPS29 and RPS7; or
f) ACTR3, ARF1, CCT4, CCT6A, COPS2, EIF2AK2, EPRS, HDGF, HIST1H1D, HIST1H4C, HNRNPA2B1, HSP90B1, LAMP2, MRPL13, PAPOLA, PPM1G, RHOA, SLC38A1, SMARCA5, SRP72, SSR3, TCP1, TRAM1 and TXNL1; or
g) ACTB, ACTN4, AP2S1, APH1A, ATP5I, ATP5J2, ATP5L, C19orf48, C4orf48, C6orf62, C8orf59, CDC37, CDKN2A, CEBPB, CFL1, CHCHD2, CHCHD3, CKB, COX5B, COX6A1, COX6C, COX7A2, COX7B, DANCR, DUSP18, EEF1B2, EEF1D, FAU, FTL, FXYD5, GPX1, GSTP1, HAP1, HNRNPA1, JUP, LGALS1, LHCGR, LSM4, LSM7, METTL12, MIF, MLF2, MRPS12, MRPS26, MT-ATP6, MT-ATP8, MT-CO1, MT-CO2, MT-CO3, MT-CYB, MT-ND1, MT-ND2, MT-ND4, MT-ND4L, MT-ND5, MT-ND6, MT-RNR1, MT-TL1, MT-TV, MTRNR2L1, MTRNR2L11, MTRNR2L12, MTRNR2L13, MTRNR2L2, MTRNR2L3, MTRNR2L5, MTRNR2L6, MTRNR2L7, MTRNR2L8, MZT2B, NDUFA11, NDUFA13, NDUFA2, NDUFB1, NDUFB4, OAZ1, PCBP1, PCBP2, PET100, PFDN5, PFN1, POLR2J, PPDPF, PRDX2, PSMB3, RAB32, RHOC, RMRP, RNA18S5, RNA28S5, RNY1, ROMO1, RPL10, RPL12, RPL13, RPL13A, RPL15, RPL18, RPL18A, RPL23A, RPL24, RPL26, RPL27A, RPL28, RPL29, RPL31, RPL32, RPL34, RPL35, RPL36, RPL36AL, RPL39, RPL41, RPL6, RPL7A, RPL8, RPL9, RPN2, RPS14, RPS15, RPS15A, RPS18, RPS19, RPS27, RPS27A, RPS28, RPS29, RPS3, RPS3A, RPS4X, RPS7, RPS9, S100A11, S100A13, SERF2, SNHG5, SSR4, SYNGR2, TAGLN2, TIMM8B, TMA7, TMSB10, TMSB4X, TRIM28, TTC19, TUBA1A, TUBA1B, UBA52 and UBL5; or
h) ABI2, ACBD3, ACIN1, ACSL4, ACTR3, ADK, ADSS, AMD1, ANKRD17, APEX1, AQR, ARF1, ARFGEF2, ARHGEF12, ARPC5, ASH1L, ASPH, ATL3, ATP1A1, ATP2A2, BNIP3, BROX, BRWD1, C1orf21, CAAP1, CANX, CAPN2, CAPRIN1, CBX5, CCDC59, CCDC90B, CCNC, CCT2, CCT4, CCT6A, CD109, CDC16, CDC5L, CDCA7L, CEBPG, CHEK1, CHML, CLIC4, CLTC, CMPK1, CNBP, COPB1, COPS2, CTTNBP2NL, CUL5, DARS, DCBLD1, DENND1B, DHRS7, DHX36, DHX40, DHX9, DICERI, DNAJC10, DNAJC3, DPH3, DST, DSTN, ECT2, EID1, EIF2AK1, EIF2AK2, EIF2S1, EIF3D, EIF4A2, EMC4, ENDOD1, ENO1, EPRS, EPS8, ERC1, ERRFI1, ETF1, EXOC3, FAM208B, FAR1, FNDC3A, FUBP1, G3BP2, GADD45GIP1, GDI2, GLTSCR2, GMPS, GOLGB1, GTPBP4, H2AFY, H3F3B, HADHA, HDGF, HDLBP, HIST1H1B, HIST1H1C, HIST1H1D, HIST1H1E, HIST1H4C, HIST2H2AC, HNRNPA2B1, HNRNPUL1, HSP90B1, HSPA5, HSPH1, IARS, ILF3, INO80D, IQGAP1, ITGA2, KIAA0368, KIAA0947, KIAA1429, KIF1B, KIF2A, LAMP2, LARP4, LARS, LCOR, LEO1, LPP, LRPPRC, LTN1, MAN1A2, MAP1LC3B, March7, MARK3, MBD4, MBNL1, MBNL2, MCUR1, MDN1, MGA, MGEA5, MIB1, MOBIB, MORF4L2, MRPL13, MRPL42, MRPS35, MSANTD3, MYCBP2, MYSM1, NASP, NCL, NEDD4L, NET1, NFX1, NOL11, NSMCE2, NUCKS1, NUDCD1, NUDT21, PAIP2, PAPOLA, PBX1, PCYT1A, PDCD10, PDE4DIP, PIK3R1, PIK3R3, PLRG1, PMAIP1, PMP22, PNPLA8, POLR2B, PPFIA1, PPM1G, PPP2R2D, PPP4R1, PRPF40A, PSMD2, PSME4, PTBP3, PTPLB, PTPN1, PTTG1IP, RAB10, RAB2A, RABEP1, RAD21, RBM28, REEP3, RHOA, RNF115, RNF168, SAP30BP, SCRN1, SEC63, SENP2, SENP6, SETD3, SETX, SF3B1, SFSWAP, SH3GLB1, SLC12A2, SLC38A1, SLC4A7, SMARCA5, SMARCC1, SMC3, SMEK1, SMEK2, SMNDC1, SNRNP200, SNX4, SOCS4, SPEN, SRP72, SRRM2, SRSF3, SSR3, SSRP1, ST3GAL1, STK3, SUCO, TAF1D, TAF2, TCP1, TIMMDC1, TIPARP, TKT, TLK1, TM9SF3, TOMM20, TOMM70A, TP53BP2, TPM1, TRA2A, TRAM1, TRIP12, TUG1, TXNL1, UAP1, UBA6, UBAP1, UBAP2, UBE2H, UBE2K, UBTF, UBXN4, UGP2, USP14, USP16, USP34, USP9X, UTP20, VIM, WDR3, XIST, XRN2, YES1, YWHAH, ZC3H14, ZDHHC20, ZFAS1, ZFYVE16 and ZNF638; or
i) any combination of up and down regulated genes in any of the Tables herein.

9. (canceled)

10. A method of detecting an immunotherapy response gene signature in a tumor comprising, detecting in tumor cells obtained from a subject in need thereof the expression or activity of a gene signature according to claim 8.

11. A method of treating a cancer in a subject in need thereof comprising detecting the expression or activity of a gene signature according to claim 8 in a biological sample comprising tumor cells obtained from the subject and administering a treatment,
wherein if a gene signature associated with non-response to an immunotherapy is detected the treatment comprises administering an agent capable of reducing expression or activity of said signature or increasing expression or activity of a gene signature associated with response to an immunotherapy, and
wherein if a gene signature associated with response to an immunotherapy is detected the treatment comprises administering an immunotherapy.

12. The method of claim 11, wherein the agent modulates expression or activity of one or more genes according to claim 8, preferably,
wherein the agent is capable of targeting or binding to one or more up-regulated secreted or cell surface exposed signature genes or polypeptides, more preferably,
wherein the agent comprises a therapeutic antibody, antibody fragment, antibody-like protein scaffold, aptamer, protein, genetic modifying agent or small molecule.

13. (canceled)

14. (canceled)

15. The method of claim 11, wherein the method further comprises administering an immunotherapy to the subject administered an agent capable of reducing the expression or activity of said signature, preferably, wherein the immunotherapy comprises one or more check point inhibitors, more preferably, wherein the one or more check point inhibitors comprise anti-CTLA4, anti-PD-L1 and/or anti-PD1 therapy.

16. (canceled)

17. (canceled)

18. A method of treating a cancer in a subject in need thereof comprising administering to the subject a therapeutically effective amount of an agent:
a) capable of modulating the expression or activity of one or more signature genes or polypeptides according to claim 8; or
b) capable of targeting or binding to one or more cell surface exposed one or more signature genes or polypeptides according to claim 8; or
c) capable of targeting or binding to one or more receptors or ligands specific for a cell surface exposed one or more signature genes or polypeptides according to claim 8; or
d) comprising one or more secreted signature genes or polypeptides according to claim 8; or
e) capable of targeting or binding to one or more secreted one or more signature genes or polypeptides according to claim 8; or
f) capable of targeting or binding to one or more receptors specific for one or more secreted signature genes or polypeptides according to claim 8.

19. The method of claim 18, wherein said agent comprises a therapeutic antibody, antibody fragment, antibody-like protein scaffold, aptamer, protein, genetic modifying agent or small molecule.

20. The method of claim 18, wherein the method further comprises administering an immunotherapy to the subject, preferably, wherein the immunotherapy comprises a check point inhibitor, more preferably, wherein the checkpoint inhibitor comprises anti-CTLA4, anti-PD-L1 and/or anti-PD1 therapy.

21. (canceled)

22. (canceled)

23. The method of claim 1, wherein the metastatic solid cancer has metastasized to the brain; and/or
wherein the metastatic solid cancer has metastasized from a solid cancer selected from breast cancer, lung cancer, thyroid cancer, and uterine cancer; and/or
wherein the subject is suffering from leptomeningeal disease; and/or
wherein the subject is suffering from neoplastic meningitis, carcinomatous meningitis, lymphomatous meningitis, or leukemic meningitis.

24. (canceled)

25. (canceled)

26. (canceled)

27. The method of claim 5, wherein the immunotherapy is a PD-1 inhibitor selected from pembrolizumab and nivolumab.

28. The method of claim 5, wherein the immunotherapy is a PD-L1 inhibitor selected from atezolizumab, avelumab, and durvalumab.

29. The method of claim 5, wherein the immunotherapy is a CTLA-4 inhibitor.

30. The method of claim 29, wherein the CTLA-4 inhibitor is ipilimumab.

31. The method of claim 1, wherein detecting expression or activity of a gene signature comprises single cell RNA sequencing (scRNA-Seq), preferably, wherein the scRNA-Seq comprises Seq-Well.

32. (canceled)

33. A method of treating cancer comprising depleting T cells having a non-responder gene signature.

34. A kit comprising reagents to detect at least one signature gene or polypeptide according to claim 8.

35. The method of claim 1, wherein the gene signature is detected in at least two time points post-treatment.


SEQUENCE LISTING (5 sequences)

SEQ ID NO: 1 - protein, 107 aa, Homo sapiens
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 2 - protein, 9 aa, Homo sapiens
IEVMYPPPY

SEQ ID NO: 3 - protein, 107 aa, Homo sapiens; identical to SEQ ID NO: 1

SEQ ID NO: 4 - protein, 288 aa, Artificial Sequence
  Synthetic polypeptide
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 5 - protein, 183 aa, Artificial Sequence
  Synthetic Polypeptide
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS